(19) United States
(12) Patent Application Publication
(10) Pub. No.: US 2024/0368139 A1
(43) Pub. Date: Nov. 7, 2024

(54) NOVEL MODULATORS OF EHMT1 AND EHMT2 AND THERAPEUTIC USE THEREOF

(71) Applicant: Tango Therapeutics, Inc., Boston, MA (US)

(72) Inventors: Joseph P. Vacca, Telford, PA (US); John P. Maxwell, Hingham, MA (US); Brett Williams, Milton, MA (US); Jon H. Come, Cambridge, MA (US); Scott Throner, Cambridge, MA (US)

(21) Appl. No.: 18/701,092

(22) PCT Filed: Oct. 14, 2022
(86) PCT No.: PCT/US2022/046761

§ 371 Date: Apr. 12, 2024

Related U.S. Application Data
(60) Provisional application No. 63/256,057, filed on Oct. 15, 2021.
(60) Provisional application No. 63/390,438, filed on Jul. 19, 2022.

(51) Int. Cl. C07D 405/14; A61K 31/506; A61K 31/538; A61K 31/55; A61K 31/551; A61K 45/06; C07D 405/12; C07D 413/14; C07D 487/04

(52) U.S. Cl. CPC C07D 405/14; A61K 31/506; A61K 31/538; A61K 31/55; A61K 31/551; A61K 45/06; C07D 405/12; C07D 413/14; C07D 487/04

(57) ABSTRACT

Described herein are novel compounds, compositions and methods for modulating EHMT1 and EHMT2 and treatment of diseases including cancer using such compounds, compositions, and methods.

### CROSS-REFERENCE TO RELATED APPLICATIONS

The present application claims the benefit of and priority to U.S. Provisional Application No. 63/256,057 filed Oct. 15, 2021, and to U.S. Provisional Application No. 63/390,438 filed Jul. 19, 2022, the entire contents of which are hereby incorporated by reference in their entireties for all purposes.

### FIELD OF THE INVENTION

The invention relates to compounds, compositions and methods for modulating EHMT1 and EHMT2, and for treatment of diseases including cancer.

### BACKGROUND OF THE INVENTION

Significant advances, particularly in immunotherapy, have been made in the treatment of cancers. Immune-checkpoint inhibitors, including anti-PD-1 and anti-CTLA-4 biologics, have shown clinical efficacy for some tumors, but not for many others, including CRCs (Topalian et al. N. Engl. J. Med. 2012, 366(26): 2443-2454; Brahmer et al. N. Engl. J. Med. 2012, 366 (26):2455-2465; Chung et al. J. Clin. Oncol. 2010, 28(21):3845-3490). Immune checkpoint inhibitors reactivate anti-tumor immunity through various parameters including tumor immunogenicity and the presence of tumor-infiltrating T-cells (Ribas et al. Science 2018, 359(6382): 1350-1355), which is known as a T-cell-inflamed or hot tumor microenvironment (TME). A hot TME is further characterized by high interferon (IFN) pathway activity (Garris et al. Clin. Cancer Res. 2020, 26(15): 3901-3907). In contrast, a T-cell infiltration low or “cold” TME is usually associated with poor responses to immune checkpoint blockade (ICB) therapy. Although mechanisms for poor response or resistance to current checkpoint blockade have been described (Sharma et al. Cell 2017, 168(4): 707-723), more mechanisms for tumor immune modulation are yet to be discovered. Epigenetic modification of histones has shown to be a malignant driver of several cancer types and associated with the immune cold signature (Topper et al. Nature Rev. Clin. Oncol. 2020, 17:75-90). As epigenetic changes are dynamic, it may be possible to reverse both the malignant process and/or therapy-resistant phenotype by targeting the epigenetic processes that cause malignancy and resistance to checkpoint inhibitor blockade.

Histone methyltransferases (HMTs) have recently emerged as targets of potential therapeutic value. They catalyze the methylation of histone lysines and arginines utilizing S-adenosyl-methionine (SAM) as substrate. The process can lead to either the activation or the repression of transcription (Jones, et. al Cell 2007, 128(4):683-692). Two related HMTs, EHMT1 and EHMT2, (Euchromatic histone-lysine N-methyltransferase 1 and 2 (EHMT1/2, also known as GLP and G9a, respectively) share approximately 80% sequence identity in their SET domain and play key roles in catalyzing mono- and di-methylation at the lysine 9 residue of histone 1H3 (H3K9me1/H1J3K9me2) in euchromatic regions. These histone marks are generally associated with the transcriptional repression of target genes. The involvement of EHMT1 and/or EHMT2 in many biological processes has been reported, including in embryonic development, repair DNA damage, and tumor cell growth and metastasis (Tachibana et al. Genes Dev. 2005, 19(7):815-826; Yokochi et al. Proc Natl Acad Sci 2009, 106(46):19363-19368; Huang et al. J Biol Chem 2010 285(13):9636-9641). Their dysregulation has also been shown to be associated with many human diseases, such as cancer, inflammatory diseases, blood disorders, and neurodegenerative disorders (Shanker, et. al. Epigenetics, 2013 8(1):16-22; Chen et al. Cancer Res. 2010, 70(20): 7830-7840; Chaturvedi et al. Proc Natl Acad Sci 2009, 106:18303-18308; Renneylle et al. 2015 Blood 126(16): 1930-1939). Thus, EHMT1 and/or EHMT2 may be targeted for modulation, thereby providing therapeutic opportunities for treating various diseases.

Over the past decades, a number of EHMT1/2 tool compounds have been discovered and demonstrated to have anti-tumor effects in several preclinical mouse xenograft models as both a single-agent treatment and in combination with anti-PD-L1 in several cancer types (Segovia et al., Nat Med. 2019; 25:1073-1081; Kato et al. Cancer Discovery 2020 10:980-987). There is therefore a need to develop clinical grade EHMT1/2 inhibitors for the treatment of human cancers, including treatment of immune cold tumors with or without the addition of immune checkpoint blockade, and/or for the treatment of other diseases such as sickle cell anemia and blood disorders.

### SUMMARY OF THE INVENTION

In one aspect, the invention provides a compound of formula (I), or a pharmaceutically acceptable salt thereof

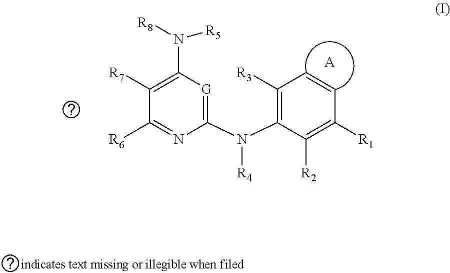

wherein,
A is an optionally substituted 5- or 6-membered oxygen-containing heterocyclic ring;
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

In one aspect, the invention provides a compound of Formula (II), or a pharmaceutically acceptable salt thereof

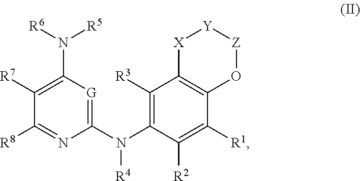

wherein X is C(R¹¹)₂, O, S(O)_{w}, or NR¹²;
Y is a bond, C(R¹³)₂, or C(R¹³)₂—C(R¹³)₂;
and Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl; each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted; each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted;
w is 0, 1, or 2.

In one aspect, the invention provides a compound of formula (II), or a pharmaceutically acceptable salt thereof

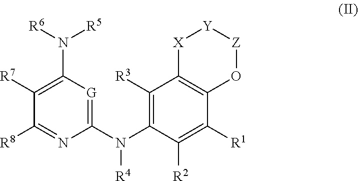

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
or R⁹ and R¹⁰ can be taken together with the carbon to which they are attached to form CO;
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂ and OH, wherein R^{E} is optionally substituted by one or more deuteriums; and
w is 0, 1, or 2.

In one aspect, the invention provides a compound of formula (II), or a pharmaceutically acceptable salt thereof

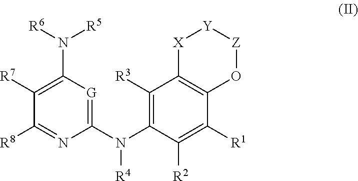

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, 3-heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen, deuterium and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, each alkyl or heteroalkyl optionally substituted with 1-5 deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂, and OH, wherein R^{E} is optionally substituted by one or more deuteriums.

The present invention also provides a compound of formula (III), or a pharmaceutically acceptable salt thereof

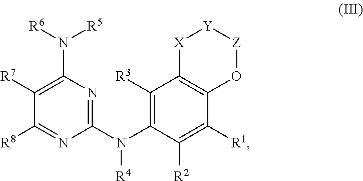

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted; e
ach R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted;
w is 0, 1, or 2.

In one aspect, the invention provides a compound of formula (III), or a pharmaceutically acceptable salt thereof

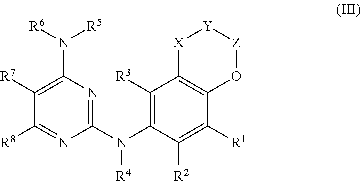

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
or R⁹ and R¹⁰ can be taken together with the carbon to which they are attached to form CO;
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂, and OH, wherein R^{E} is optionally substituted by one or more deuteriums;
w is 0, 1, or 2.

In one aspect, the invention provides a compound of formula (III), or a pharmaceutically acceptable salt thereof

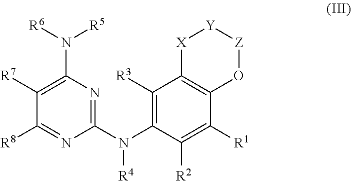

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
R′ is H or C₁-C₆ alkyl;
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; or
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, C₁-C₆ hydroxyalkyl, NH₂, and OH, wherein R^{E} is optionally substituted by one or more deuteriums;
w is 0, 1, or 2.

In one aspect, the invention provides a compound of formula (III), or a pharmaceutically acceptable salt thereof

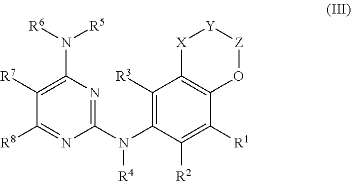

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂ and OH, wherein R^{E} is optionally substituted by one or more deuteriums.

In one aspect, the invention provides a compound of formula (IIIa-2), or a pharmaceutically acceptable salt thereof, wherein

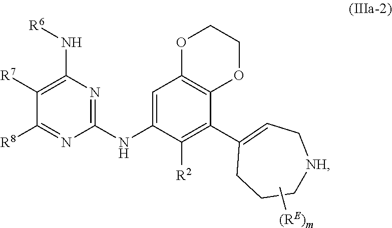

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

In one aspect, the invention provides a compound of formula (IIIb-2), or a pharmaceutically acceptable salt thereof, wherein

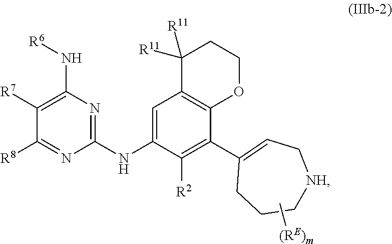

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R¹¹ is independently selected from H, C₁-C₆ alkyl, hydroxy, and halogen, or two R¹¹ are taken together with the carbon to which they are attached to form a spirofused C₃-C₇ cycloalkyl;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

In one aspect, the invention provides a compound of formula (IVb-2), or a pharmaceutically acceptable salt thereof, wherein

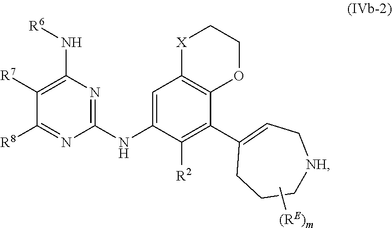

X is O or C(R¹¹)₂
R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R¹¹ is independently selected from H, C₁-C₆ alkyl, hydroxy, and halogen, or two R¹¹ are taken together with the carbon to which they are attached to form a spirofused C₃-C₇ cycloalkyl;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

In one aspect, the invention provides a compound of formula (IIIc-2), or a pharmaceutically acceptable salt thereof, wherein

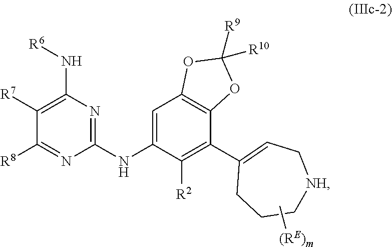

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R⁹ and R¹⁰ is independently H, C₁-C₆ alkyl, or halogen;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

In one aspect, the invention provides a compound of formula (IIId-2), or a pharmaceutically acceptable salt thereof, wherein

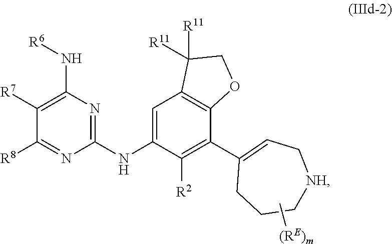

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R¹¹ is independently selected from H, C₁-C₆ alkyl, hydroxy, and halogen, or two R¹¹ are taken together with the carbon to which they are attached to form a spirofused C₃-C₇ cycloalkyl
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

In one aspect, the invention provides a compound of formula (IVa-2), or a pharmaceutically acceptable salt thereof, wherein

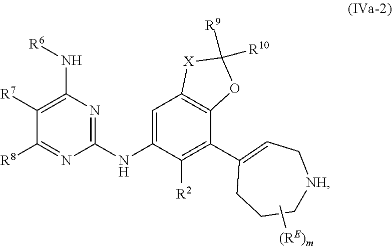

X is O or C(R¹¹)₂;
R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R⁹ and R¹⁰ is independently H, C₁-C₆ alkyl, or halogen;
each R¹¹ is independently selected from H, C₁-C₆ alkyl, hydroxy, and halogen, or two R¹¹ are taken together with the carbon to which they are attached to form a spirofused C₃-C₇ cycloalkyl
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

In one aspect, the invention provides a composition comprising a compound of any one of the compounds disclosed herein, or a pharmaceutically acceptable salt thereof, and a pharmaceutically acceptable carrier.

In another aspect, the invention provides a method of treating a disease or disorder that can be treated by modulation of EHMT1 or EHMT2, the method comprising administering to a patient in need thereof a compound described herein or a composition described herein.

In one aspect, the invention provides use of a compound as disclosed herein, or a pharmaceutically acceptable salt thereof, or a composition as disclosed herein, in the manufacture of a medicament for the treatment of a disease or disorder that can be treated by modulation of EHMT1 or EHMT2.

In one aspect, the invention provides use of a compound as disclosed herein, or a pharmaceutically acceptable salt thereof, or a composition as disclosed herein for the treatment of a disease or disorder that can be treated by modulation of EHMT1 or EHMT2.

In one aspect, the invention provides a compound as disclosed herein, or a pharmaceutically acceptable salt thereof, or a composition as disclosed herein for use in treating a disease or disorder that can be treated by modulation of EHMT1 or EHMT2.

In another aspect, provided is a use of a compound of the disclosure in the manufacture of a medicament for the treatment of cancer.

Still other objects and advantages of the invention will become apparent to those of skill in the art from the disclosure herein, which is simply illustrative and not restrictive. Thus, other embodiments will be recognized by the skilled artisan without departing from the spirit and scope of the invention.

### DETAILED DESCRIPTION OF THE INVENTION

As generally described herein, the present invention provides compounds (e.g., compounds of Formula (I), (II), (IIa), (IIb), (IIc), (III), (IIIa), (IIIb), (IIIc), (IIId), (IIa-1), (IIb-1), (IIc-1), (IId-1), (IIIa-1), (IIIb-1), (IIIc-1), (IIId-1), (IIIa-2), (IIIb-2), (IIIc-2), (IIId-2), (IVa), (IVb), (IVa-1), (IVb-1), (IVa-2) and (IVb-2) or compounds of Table 1, or pharmaceutically acceptable salts thereof) that are useful for disorders (e.g., cancer) associated with modulation of EHMT1 or EHMT2.

### COMPOUNDS

In one aspect, the invention provides a compound of formula (I), or a pharmaceutically acceptable salt thereof

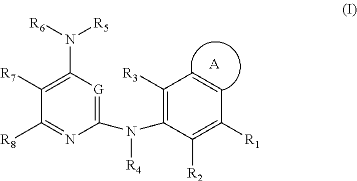

wherein,
A is an optionally substituted 5- or 6-membered oxygen-containing heterocyclic ring;
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

In one aspect, the invention provides compound of formula (I) or a pharmaceutically acceptable salt thereof

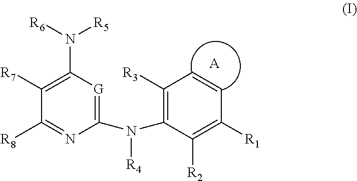

wherein,
A is an optionally substituted 5- or 6-membered oxygen-containing heterocyclic ring;
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted; each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

In some embodiments, A is an optionally substituted 5-membered oxygen-containing heterocyclic ring. In some embodiments, A is an optionally substituted 6-membered oxygen-containing heterocyclic ring. In some embodiments, A has 1 or 2 oxygens as the only ring heteroatoms. In some embodiments, A has 1 oxygen as the only ring heteroatom.

In one aspect, the invention provides a compound of Formula (II), or a pharmaceutically acceptable salt thereof

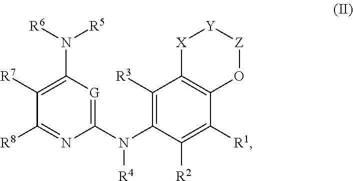

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰;
or X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

In one aspect, the invention provides a compound of Formula (II), or a pharmaceutically acceptable salt thereof

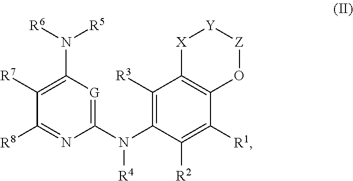

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰;
or X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

In one embodiment, provided is a compound of formula (II)

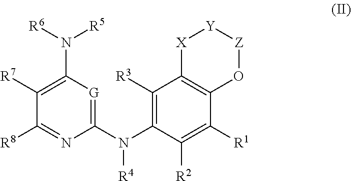

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH;
w is 0, 1, or 2.

Also provided herein, in certain embodiments, is a compound of formula (II), or a pharmaceutically acceptable salt thereof

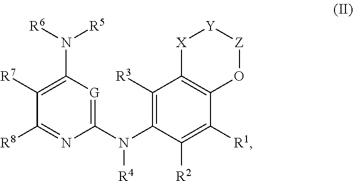

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E}; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH, wherein R^{E} is optionally substituted by one or more deuteriums;
w is 0, 1, or 2.

In one aspect, the invention provides a compound of formula (II), or a pharmaceutically acceptable salt thereof

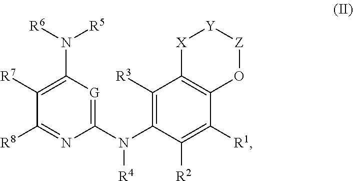

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
or R⁹ and R¹⁰ can be taken together with the carbon to which they are attached to form CO;
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂ and OH, wherein R^{E} is optionally substituted by one or more deuteriums; and
w is 0, 1, or 2.

In some embodiments, provided is a compound of formula (II)

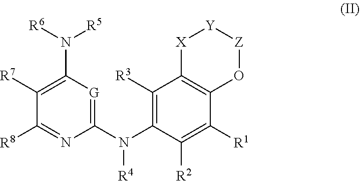

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, and OH.

In certain embodiments, provided herein is a compound of formula (II), or a pharmaceutically acceptable salt thereof

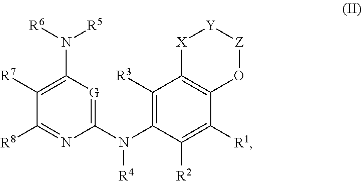

wherein X is C(R¹¹)₂, O, or NR¹²;
Y is a bond or C(R¹³)₂, and Z is CR⁹R¹⁰; or
X—Y is C(R¹¹)═C(R¹³) and Z is CR⁹R¹⁰; or
X—Y—Z is C(R¹¹)═C(R¹³); G is N or CR⁷;
R¹ is —O—C₁-C₆ alkylene-E, —NR¹²—C₁-C₆ alkylene-E, or E; E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen; R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen, each alkyl or heteroalkyl optionally substituted with 1-5 deuteriums;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, and OH, R^{E} optionally substituted by one or more deuteriums.

In one aspect, the invention provides a compound of formula (II), or a pharmaceutically acceptable salt thereof

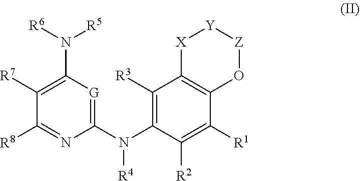

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, 3-heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen, deuterium and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, each alkyl or heteroalkyl optionally substituted with 1-5 deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂, and OH, wherein R^{E} is optionally substituted by one or more deuteriums.

In some embodiments, G is CR⁷. In some embodiments, G is CH. In some embodiments, G is N.

Provided herein, in certain embodiments, is a compound of formula (III), or a pharmaceutically acceptable salt thereof

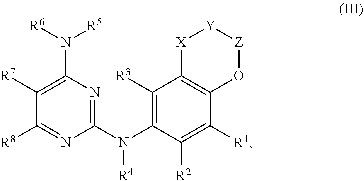

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted;
w is 0, 1, or 2.

Provided herein, in certain embodiments, is a compound of formula (III), or a pharmaceutically acceptable salt thereof

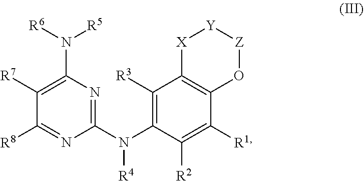

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted;
w is 0, 1, or 2.

In some embodiments, provided is a compound of formula (III)

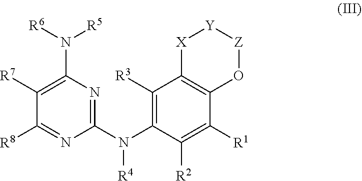

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH;
w is 0, 1, or 2.

In certain embodiments, provided herein is a compound of formula (III), or a pharmaceutically acceptable salt thereof

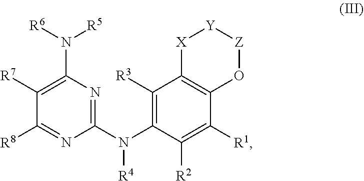

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³); R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH, wherein R^{E} is optionally substituted by one or more deuteriums; w is 0, 1, or 2.

In one aspect, the invention provides a compound of formula (III), or a pharmaceutically acceptable salt thereof

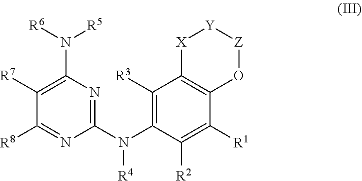

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
or R⁹ and R¹⁰ can be taken together with the carbon to which they are attached to form CO;
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂, and OH, wherein R^{E} is optionally substituted by one or more deuteriums;
w is 0, 1, or 2.

In some embodiments, provided is a compound of formula (III)

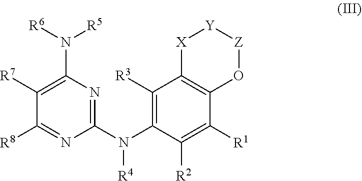

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; or
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, and OH;
w is 0, 1, or 2.

In certain embodiments, provided herein is a compound of formula (III), or a pharmaceutically acceptable salt thereof

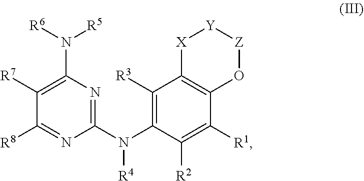

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
R′ is H or C₁-C₆ alkyl; each R² and R³ is independently H, C₁-C₆ alkyl, C₁-C₆ haloalkyl, C₁-C₆ heteroalkyl, hydroxy, cyano, or halogen; R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, and OH, wherein R^{E} is optionally substituted by one or more deuteriums; and
w is 0, 1, or 2.

In one aspect, the invention provides a compound of formula (III), or a pharmaceutically acceptable salt thereof

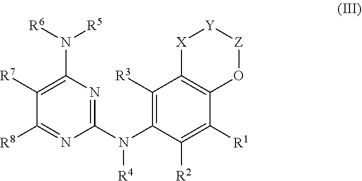

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
R′ is H or C₁-C₆ alkyl;
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, C₁-C₆ hydroxyalkyl, NH₂, and OH, wherein R^{E} is optionally substituted by one or more deuteriums;
w is 0, 1, or 2.

In some embodiments, provided is a compound of formula (III)

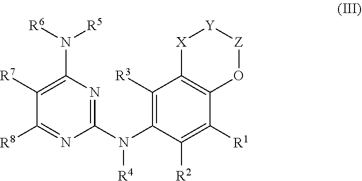

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, and OH.

In certain embodiments, provided herein is a compound of formula (III), or a pharmaceutically acceptable salt thereof

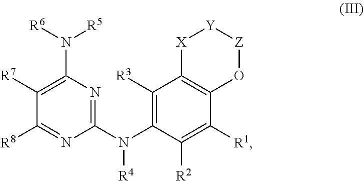

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E}; each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl; each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, and OH, wherein C₁-C₆ alkyl is optionally substituted by one or more deuteriums.

In one aspect, the invention provides a compound of formula (III), or a pharmaceutically acceptable salt thereof

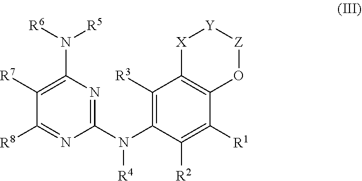

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂ and OH, wherein R^{E} is optionally substituted by one or more deuteriums.

As generally defined herein, X is C(R¹¹)₂, O, S(O)_{w}, or NR¹², wherein R¹¹, R¹² and w are as defined herein.

In some embodiments, X is C(R¹¹)₂, O, or NR¹².

In some embodiments, X is C(R¹¹)₂ or O. In some embodiments, X is CHR¹¹, CH₂ or O.

In some embodiments, X is CH(CH₃), CH₂ or O. In some embodiments, X is CH(CH₃), or O. In some embodiments, X is CH₂ or O.

In some embodiments, X is C(R¹¹)₂.

In some embodiments, X is CH₂. In some embodiments, X is CH(CH₃).

In other embodiments, X is O.

In other embodiments, X is NR¹².

As generally defined herein, Y is a bond, C(R¹³)₂, or C(R¹³)₂—C(R¹³)₂, wherein R¹¹ and R¹³ are as defined herein.

In some embodiments, Y is C(R¹³)₂. In some embodiments, Y is a bond or C(R¹³)₂. In some embodiments, Y is a bond or CH₂.

In some embodiments, Y is CH₂.

In other embodiments, Y is a bond.

In some embodiments, X—Y is C(R¹¹)═C(R¹³) and Z is CR⁹R¹⁰, wherein R⁹, R¹⁰, R¹¹ and R¹³ are as defined herein.

In some embodiments, X—Y is CH═CH.

In some embodiments, X—Y—Z is CH═CH.

In some embodiments, X is C(R¹¹)₂ (e.g., CH₂) and Y is C(R¹³)₂ (e.g., CH₂).

In some embodiments, X is CH₂ and Y is CH₂.

In other embodiments, X is C(R¹¹)₂ (e.g., CH₂) and Y is a bond.

In some embodiments, X is 0 and Y is C(R¹³)₂ (e.g., CH₂).

In some embodiments, X is 0 and Y is CH₂.

In other embodiments, X is 0 and Y is a bond.

As generally defined herein, Z is CR⁹R¹⁰ wherein R⁹ and R¹⁰ are as defined herein. In some embodiments, Z is CR⁹R¹⁰ and each R⁹ and R¹⁰ is independently H, C₁-C₆ alkyl, or halogen. In some embodiments, Z is CR⁹R¹⁰ and each R⁹ and R¹⁰ is independently H, Me or F.

In some embodiments, Z is selected from CH₂, CF₂, and CMe₂.

In some embodiments, Z is selected from C═O, CF₂ and CH₂. In some embodiments, Z is ═O. In some embodiments, Z is CH₂. In some embodiments, Z is CF₂.

In some embodiments, X—Y—Z is C(R¹¹)═C(R¹³), wherein R⁹, R¹, R¹¹, R¹³ are as defined herein.

As generally defined herein, E is C₃-C₁₀ cycloalkyl or C₃-C₁₀ heterocycloalkyl, each of which is optionally substituted.

In some embodiments, E is C₃-C₁₀ cycloalkyl or C₃-C₁₀ heterocycloalkyl, each of which is optionally substituted with 1-4 R^{E} (i.e., 0, 1, 2, 3 or 4 R^{E}), wherein each R^{E} is as defined herein. In some embodiments, the C₃-C₁₀ cycloalkyl or C₃-C₁₀ heterocycloalkyl are unsubstituted. In some embodiments, the C₃-C₁₀ cycloalkyl or C₃-C₁₀ heterocycloalkyl are substituted with 1 R^{E}. In some embodiments, the C₃-C₁₀ cycloalkyl or C₃-C₁₀ heterocycloalkyl are substituted with 2 R^{E}. In some embodiments, the C₃-C₁₀ cycloalkyl or C₃-C₁₀ heterocycloalkyl are substituted with 3 R^{E}. In some embodiments, the C₃-C₁₀ cycloalkyl or C₃-C₁₀ heterocycloalkyl are substituted with 4 R^{E}.

In some embodiments, E is C₃-C₁₀ cycloalkyl optionally substituted with 1-4 R^{E} (i.e., substituted with 0, 1, 2 or 3 R^{E}).

In some embodiments, E is cyclohexyl or cyclohexenyl optionally substituted with 1-4 R^{E} (i.e., substituted with 0, 1, 2 or 3 R^{E}).

In other embodiments, E is heterocycloalkyl optionally substituted with 1-4 R^{E} (i.e., substituted with 0, 1, 2 or 3 R^{E}).

In some embodiments, E is C₃-C₁₀ heterocycloalkyl optionally substituted with 1-4 R^{E}(i.e., substituted with 0, 1, 2 or 3 R^{E}).

In some embodiments, the heterocycloalkyl has 3-10 ring atoms including 1-3 ring heteroatoms selected from N, O, and S.

In some embodiments, the heterocycloalkyl has 5-8 ring atoms including 1-3 ring heteroatoms selected from N, O, and S.

In some embodiments, the heterocycloalkyl has 5-8 ring atoms including 1 or 2 nitrogen heteroatoms.

In some embodiments, the heterocycloalkyl has 5-8 ring atoms including 1 nitrogen heteroatom.

In some embodiments, the heterocycloalkyl has 6-8 ring atoms including 1-3 ring heteroatoms selected from N, O, and S.

In some embodiments, the heterocycloalkyl has 6-8 ring atoms including 1 or 2 nitrogen heteroatoms.

In some embodiments, the heterocycloalkyl has 6-8 ring atoms including 1 nitrogen heteroatom.

In some embodiments, E is selected from pyrrolidinyl, piperidinyl, piperazinyl, tetrahydropyridinyl, azepanyl, diazepanyl, tetrahydro-1H-azepinyl, 2,6-diazaspiro[3.5]nonanyl, 2,6-diazaspiro[3.4]octanyl, hexahydrocyclopenta[c]pyrrolyl, 1,8-diazaspiro[4.5]decanyl, 1,7-diazaspiro[4.4]nonanyl, 1,7-diazaspiro[4.5]decanyl, 2,7-diazaspiro[4.4]nonanyl, 2,8-diazaspiro[4.5]decanyl, 2,7-diazaspiro[4.5]decanyl, cyclohexenyl, octahydrocyclopenta[c]pyrrolyl and octahydropyrrolo[3,4-c]pyrrolyl, each optionally substituted with 1-4 R^{E} (i.e., substituted with 0, 1, 2 or 3 R^{E}).

In some embodiments, E is selected from pyrrolidinyl, piperidinyl, piperazinyl, tetrahydropyridinyl, azepanyl, diazepanyl, tetrahydro-1H-azepinyl, cyclohexenyl, hexahydrocyclopenta[c]pyrrolyl, octahydrocyclopenta[c]pyrrolyl and octahydropyrrolo[3,4-c]pyrrolyl, each optionally substituted with 1-4 R^{E} (i.e., substituted with 0, 1, 2 or 3 R^{E}).

In some embodiments, E is selected from pyrrolidinyl and tetrahydro-1H-azepinyl, each optionally substituted with 1-4 R^{E}(i.e., substituted with 0, 1, 2 or 3 R^{E}).

In some embodiments, E is pyrrolidinyl, optionally substituted with 1-4 R^{E} (i.e., substituted with 0, 1, 2 or 3 R^{E}).

In some embodiments, E is tetrahydro-1H-azepinyl optionally substituted with 1-4 R^{E}(i.e., substituted with 0, 1, 2 or 3 R^{E}).

In some embodiments, E is selected from pyrrolidine-1-yl, piperidin-1-yl, piperidin-4-yl, piperazin-1-yl, tetrahydropyridin-4-yl, azepan-4-yl, 1,4-diazepan-1-yl, 2,3,4,7-tetrahydro-1H-azepin-5-yl, cyclohexen-1-yl, 2,6-diazaspiro[3.5]nonan-2-yl, 2,6-diazaspiro[3.4]octan-2-yl, 1,8-diazaspiro[4.5]decan-8-yl, 1,7-diazaspiro[4.4]nonan-7-yl, 1,7-diazaspiro[4.5]decan-7-yl, 2,7-diazaspiro[4.4]nonan-2-yl, 2,8-diazaspiro[4.5]decan-2-yl, 2,7-diazaspiro[4.5]decan-2-yl, 2,3,6,7-tetrahydro-1H-azepin-4-yl, 1,2,3,3a,4,6a-hexahydrocyclopenta[c]pyrrol-5-yl, octahydrocyclopenta[c]pyrol-5-yl and octahydropyrrolo[3,4-c]pyrrol-2-yl, each optionally substituted with 1-4 R^{E} (i.e., substituted with 0, 1, 2 or 3 R^{E}).

In some embodiments, E is selected from pyrrolidine-1-yl, piperidin-1-yl, piperidin-4-yl, piperazin-1-yl, tetrahydropyridin-4-yl, azepan-4-yl, 1,4-diazepan-1-yl, 2,3,4,7-tetrahydro-1H-azepin-5-yl, cyclohexen-1-yl, 1,2,3,3a,4,6a-hexahydrocyclopenta[c]pyrrol-5-yl, octahydrocyclopenta[c]pyrol-5-yl and octahydropyrrolo[3,4-c]pyrrol-2-yl, each optionally substituted with 1-4 R^{E} (i.e., substituted with 0, 1, 2 or 3 R^{E}).

In some embodiments, E is selected from pyrrolidine-1-yl and 2,3,4,7-tetrahydro-1H-azepin-5-yl, each optionally substituted with 1-4 R^{E} (i.e., substituted with 0, 1, 2 or 3 R^{E}).

In some embodiments, E is pyrrolidine-1-yl, optionally substituted with 1-4 R^{E} (i.e., substituted with 0, 1, 2 or 3 R^{E}).

In some embodiments, E is 2,3,4,7-tetrahydro-1H-azepin-5-yl optionally substituted with 1-4 R^{E} (i.e., substituted with 0, 1, 2 or 3 R^{E}).

In some embodiments, E is

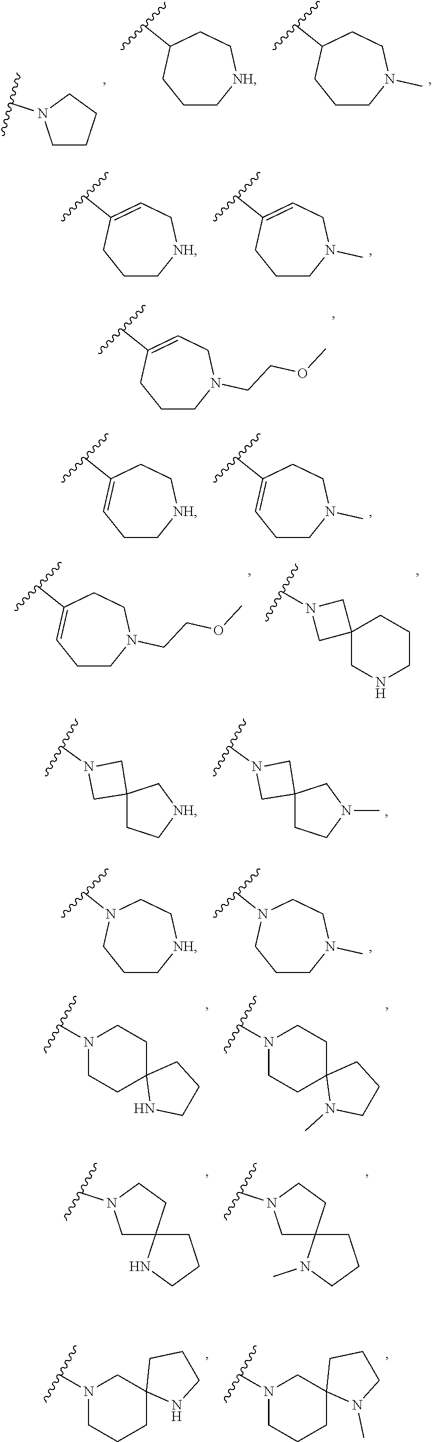

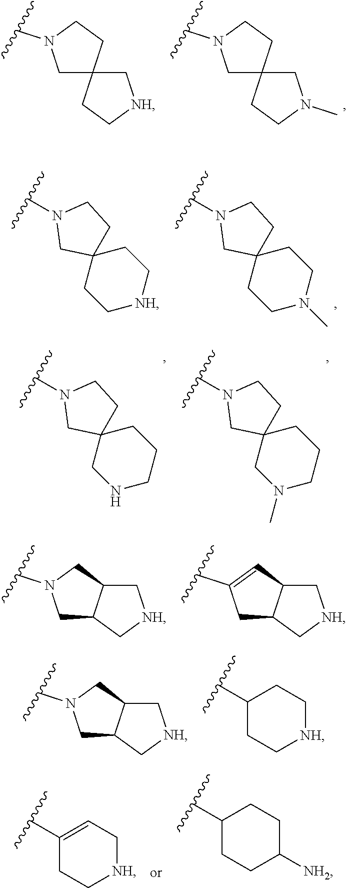

optionally substituted with 1-3 R^{E}.

In some embodiments, E is

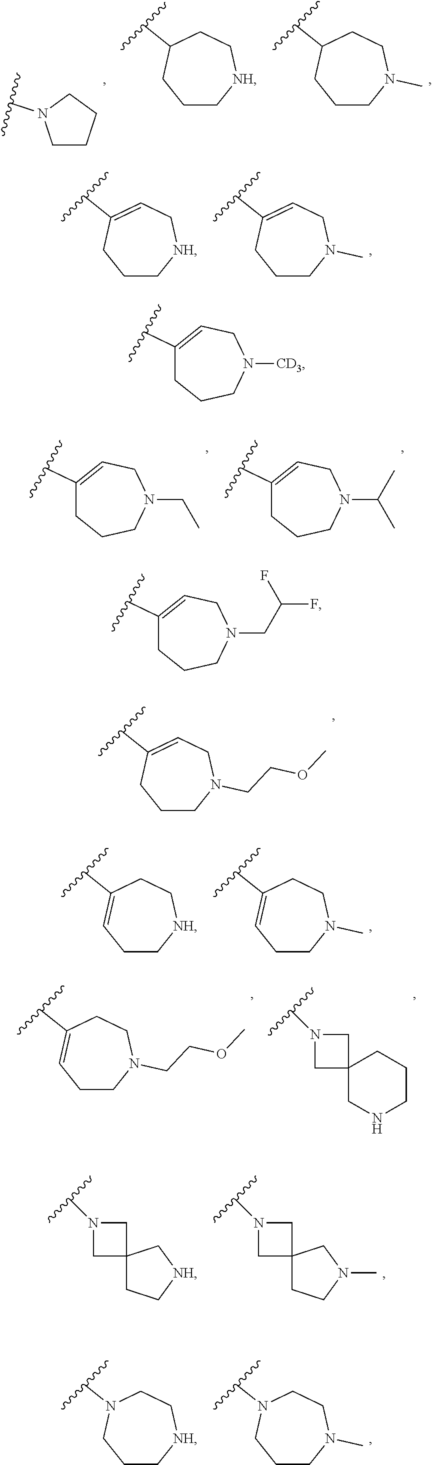

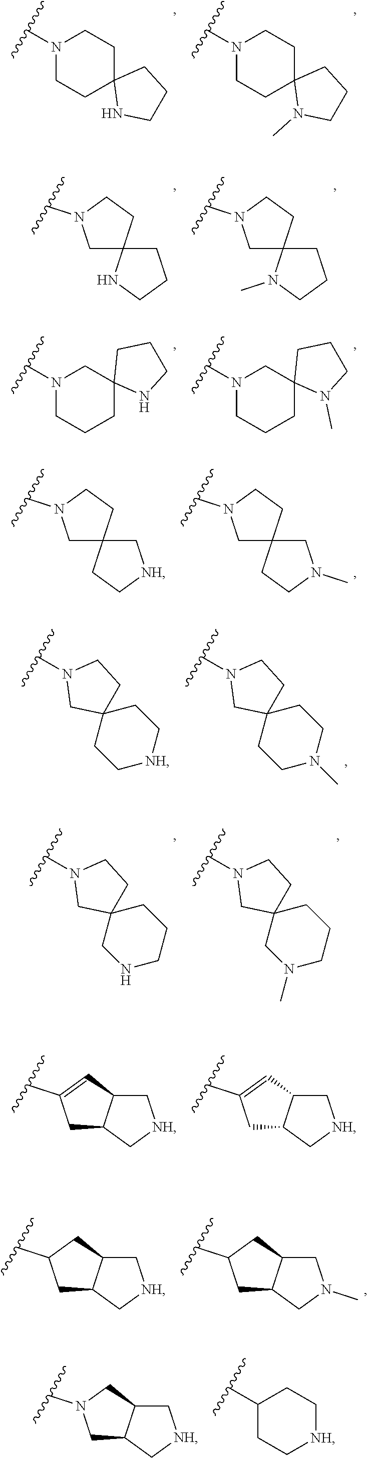

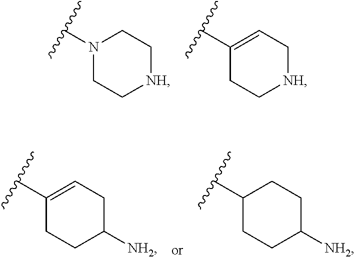 [SMILES: *N1CCNCC1 ; *C1=CCNCC1 ; *C1=CCC(N)CC1 ; *C1CCC(N)CC1]

optionally substituted with 1-3 R^{E}.

In some embodiments, E is

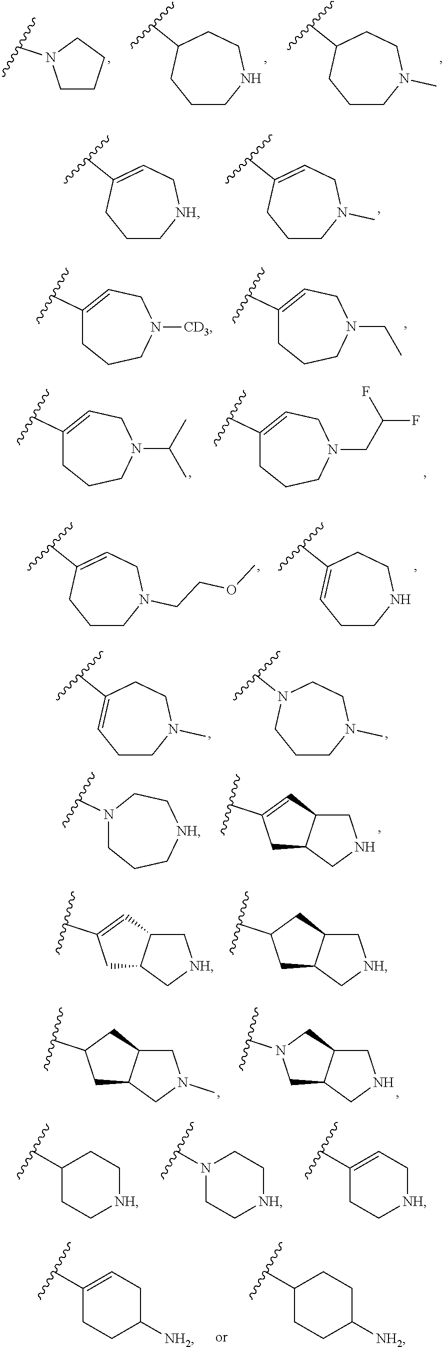

optionally substituted with 1-3 R^{E}.

In some embodiments, E is

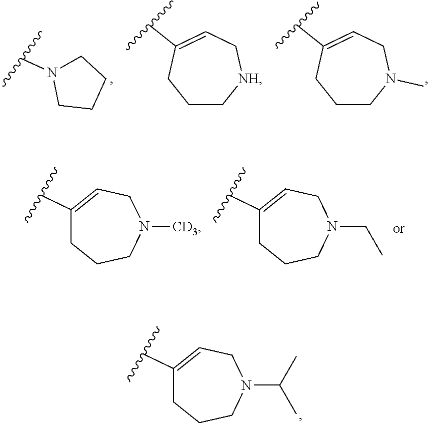

optionally substituted with 1-3 R^{E}.

In some embodiments, E is

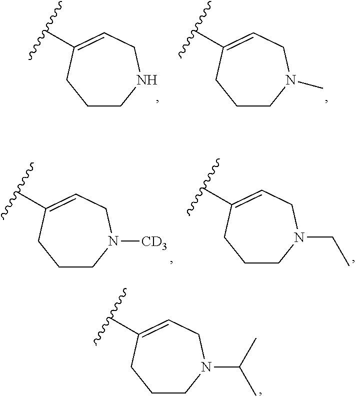

optionally substituted with 1-3 R^{E}.

In some embodiments, E is

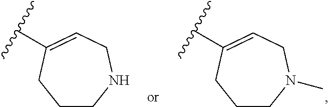

optionally substituted with 1-3 R^{E}.

In some embodiments, E is

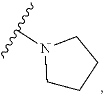 [SMILES: *N1CCCC1]

optionally substituted with 1-3 R^{E}. In some embodiments, E is

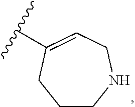 [SMILES: *C1=CCNCCC1]

optionally substituted with 1-3 R^{E}. In some embodiments, E is

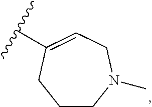 [SMILES: *C1=CCN(C)CCC1]

optionally substituted with 1-3 R^{E}. In some embodiments, E is

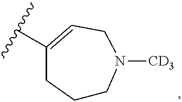 [SMILES: *C1=CCN(C([2H])([2H])[2H])CCC1]

optionally substituted with 1-3 R^{E}. In some embodiments, E is

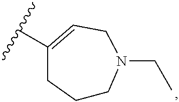 [SMILES: *C1=CCN(CC)CCC1]

optionally substituted with 1-3 R^{E}. In some embodiments, E is

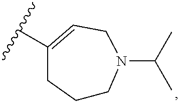 [SMILES: *C1=CCN(C(C)C)CCC1]

optionally substituted with 1-3 R^{E}.

In some embodiments, E is

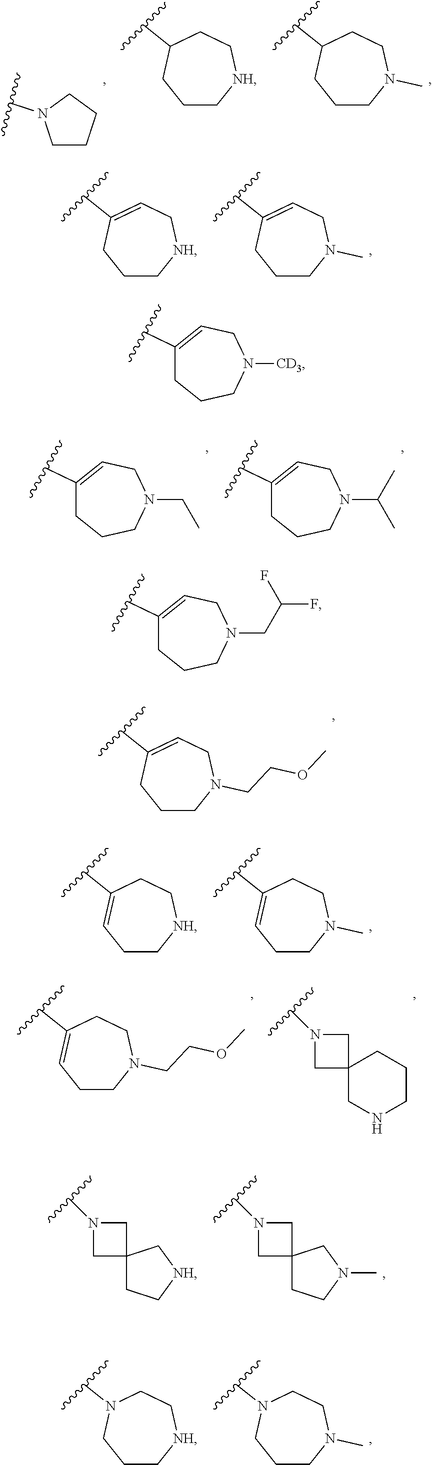

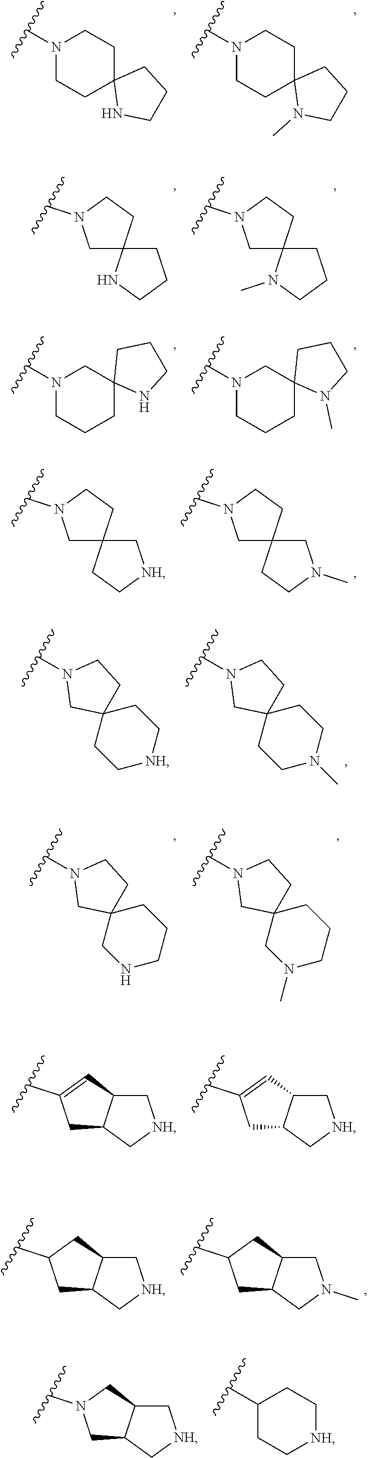

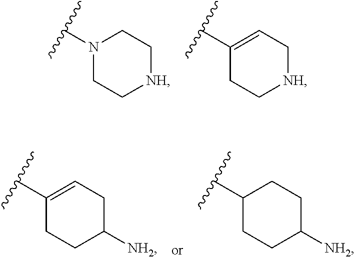 [SMILES: *N1CCNCC1 ; *C1=CCNCC1 ; *C1=CCC(N)CC1 ; *C1CCC(N)CC1]

In some embodiments, E is

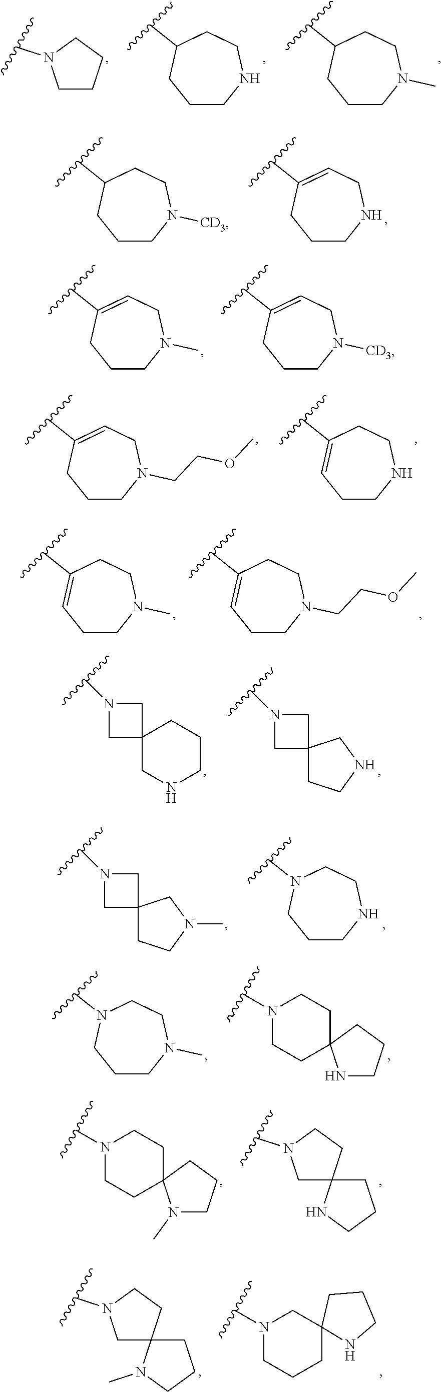

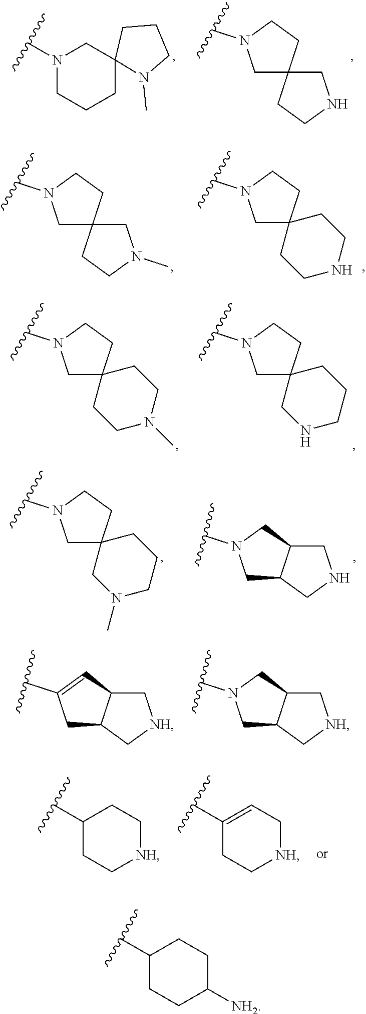

In some embodiments, E is

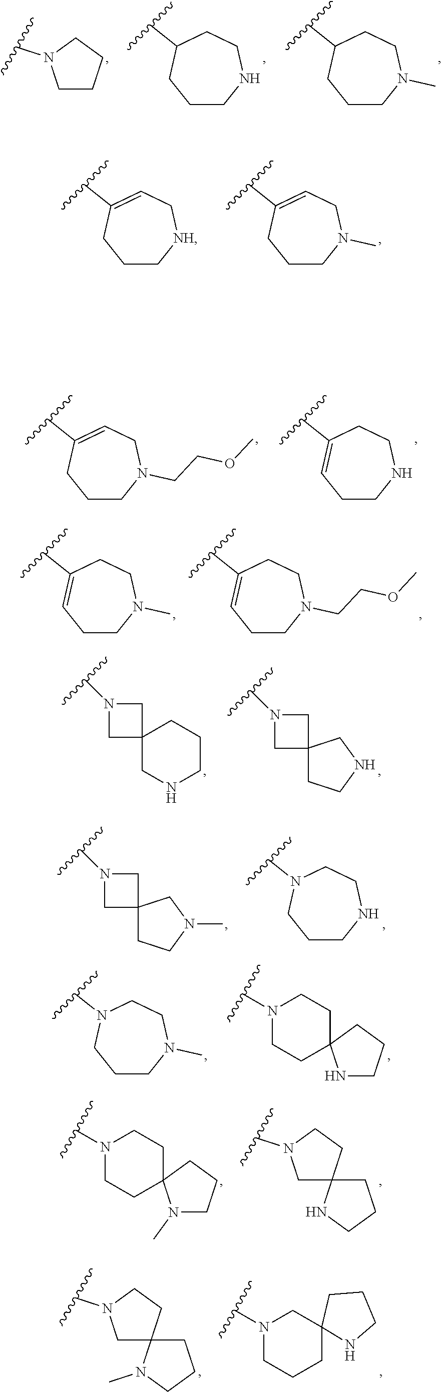

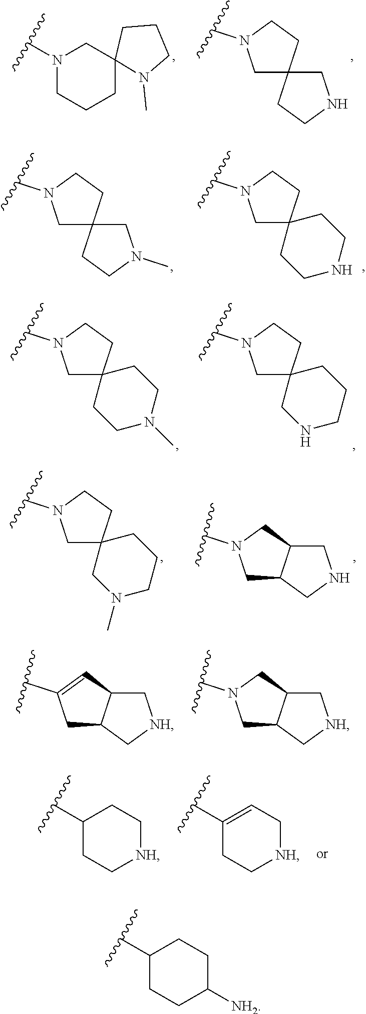

In some embodiments, E is

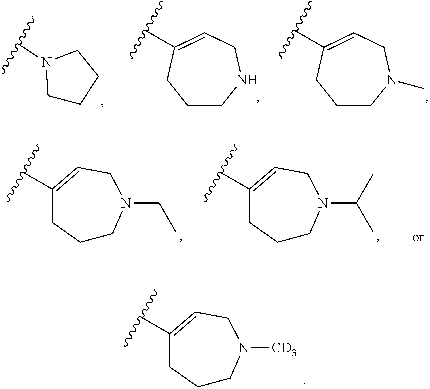

In some embodiments, E is

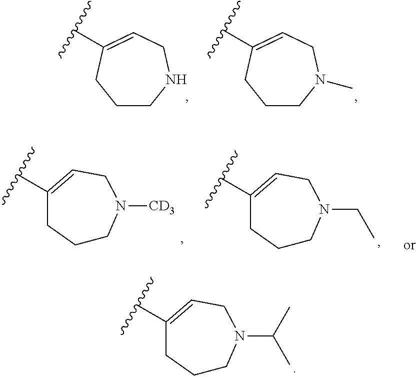

In some embodiments, E is

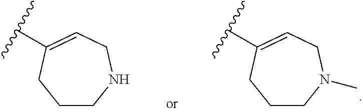

In some embodiments, E is

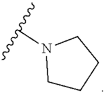 [SMILES: *N1CCCC1]

In some embodiments, E is

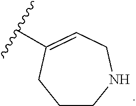 [SMILES: *C1=CCNCCC1]

In some embodiments, E is

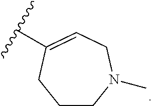 [SMILES: *C1=CCN(C)CCC1]

In some embodiments, E is

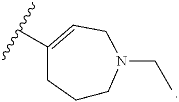 [SMILES: *C1=CCN(CC)CCC1]

In some embodiments, E is

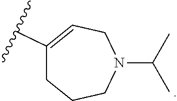 [SMILES: *C1=CCN(C(C)C)CCC1]

In some embodiments, E is

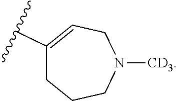 [SMILES: *C1=CCN(C([2H])([2H])[2H])CCC1]

In some embodiments, E is

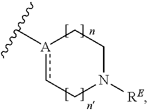

wherein [symbol] is a single bond and A is CH or N; or [symbol] is a double bond and A is C; E is optionally substituted by 1-3 additional R^{E}; n is 1 or 2; and n′ is 1 or 2.

As generally defined herein, each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂ and OH, wherein R^{E} is optionally substituted by one or more deuteriums (i.e., substituted by 0, 1, 2, 3, 4 or 5 deuteriums).

In some embodiments, each R^{E} is independently selected from halogen, C₁-C₆ alkyl, C₁-C₆ haloalkyl, C₁-C₆ alkoxy, C₃-C₇ cycloalkyl, heterocyclyl and OH, wherein R^{E} is optionally substituted by one or more deuteriums (i.e., substituted by 0, 1, 2, 3, 4 or 5 deuteriums).

In some embodiments, each R^{E} is independently selected from halogen, C₁-C₆ alkyl, C₁-C₆ haloalkyl, C₁-C₆ heteroalkyl, C₁-C₆ alkoxy, and OH, wherein R^{E} is optionally substituted by one or more deuteriums (i.e., substituted by 0, 1, 2, 3, 4 or 5 deuteriums).

In some embodiments, each R^{E} is independently selected from halogen, C₁-C₆ alkyl, C₁-C₆ haloalkyl, C₁-C₆ alkoxy, C₁-C₆ heteroalkyl, C₁-C₆ hydroxyalkyl, NH₂ and OH, wherein R^{E} is optionally substituted with 1-5 deuteriums (i.e., substituted by 0, 1, 2, 3, 4 or 5 deuteriums).

In some embodiments, each R^{E} is independently selected from halogen, C₁-C₆ alkyl, C₁-C₆ haloalkyl, C₁-C₆ alkoxy, and OH, wherein R^{E} is optionally substituted with 1-5 deuteriums (i.e., substituted by 0, 1, 2, 3, 4 or 5 deuteriums). In some embodiments, each R^{E} is independently selected from halogen, C₁-C₆ alkyl, C₁-C₆ haloalkyl, C₁-C₆ alkoxy, and OH.

In some embodiments, each R^{E} is independently selected from C₁-C₆alkyl and OH, wherein R^{E} is optionally substituted with 1-5 deuteriums (i.e., substituted by 0, 1, 2, 3, 4 or 5 deuteriums).

In some embodiments, each R^{E} is independently selected from halogen, C₁-C₆ alkyl and OH, wherein R^{E} is optionally substituted with 1-5 deuteriums (i.e., substituted by 0, 1, 2, 3, 4 or 5 deuteriums).

In some embodiments, each R^{E} is independently selected from Me, CD₃, Et, iPr, F, OH, OMe, CH₂OH, CH₂CHF₂, CHF₂, CH₂F, CH₂CH₂OMe and NH₂.

In some embodiments, each R^{E} is independently selected from Me, CD₃, Et, F and OH In some embodiments, each R^{E} is independently selected from Me, CD₃ and OH.

In some embodiments, each R^{E} is independently selected from Me and OH.

In some embodiments, each R^{E} is independently F. In some embodiments, each R^{E} is independently CD₃. In some embodiments, each R^{E} is independently Me. In some embodiments, each R^{E} is independently OH.

In some embodiments, R^{E} is attached to a carbon atom.

In some embodiments, R^{E} is attached to a nitrogen atom.

As generally defined herein, each R′ is independently selected from H and C₁-C₆ alkyl.

In some embodiments, each R′ is independently selected from H and Me. In some embodiments, R′ is H. In some embodiments, R′ is Me.

As generally defined herein, R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 (i.e., substituted with 0, 1, 2 or 3) individually selected halo or C₁-C₆ alkyl, wherein R¹² and R′ are as defined herein.

In some embodiments, R¹ is —O—C₁-C₆ alkylene-E, —NR¹²—C₁-C₆ alkylene-E, or E, wherein each methylene group in C₁-C₆ alkylene is individually optionally replaced by O or NR′.

In some embodiments, R¹ is —O—C₁-C₆ alkylene-E, —NR¹²—C₁-C₆ alkylene-E, or E.

In some embodiments, R¹ is —O—C₁-C₆ alkylene-E.

In some embodiments, R¹ is —NR¹²—C₁-C₆ alkylene-E.

In some embodiments, R¹ is selected from E and —O—C₁-C₆ alkylene-E.

In some embodiments, R¹ is selected from —O—CH₂—CH₂—CH₂-E, —O—CH₂—CH₂-E and E.

In some embodiments, R¹ is selected from —O—(CH₂)₂-E and —O—(CH₂)₃-E.

In some embodiments, R¹ is E.

In some embodiments, R¹ is —O—CH₂—CH₂—CH₂-E.

In some embodiments, R¹ is selected from

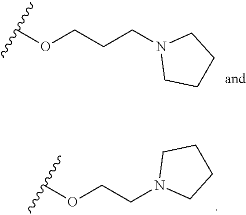 [SMILES: *OCCCN1CCCC1 ; *OCCN1CCCC1]

In some embodiments, R¹ is

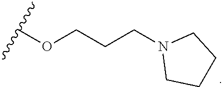 [SMILES: *OCCCN1CCCC1]

In some embodiments, R¹ is selected from

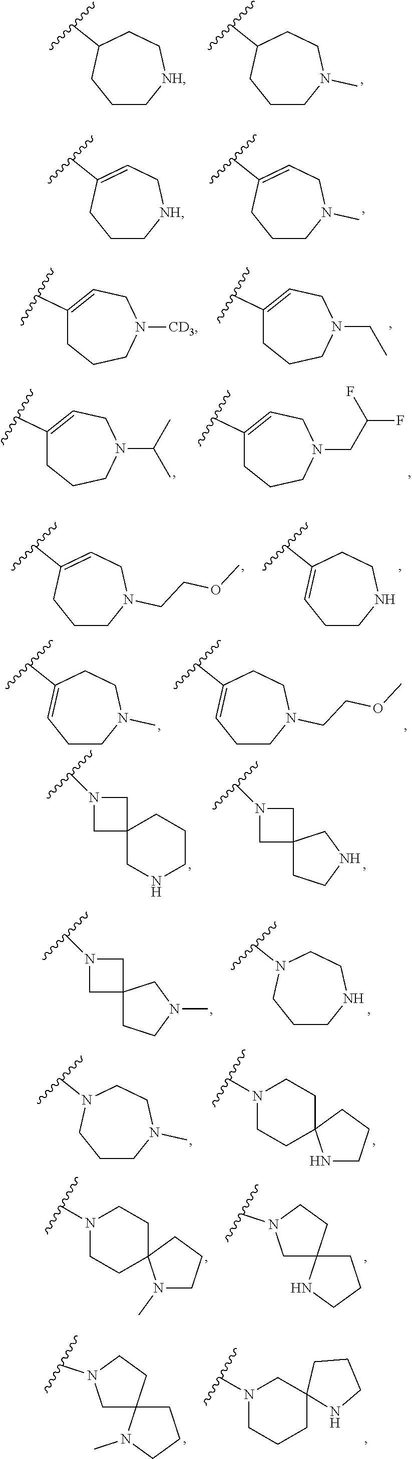

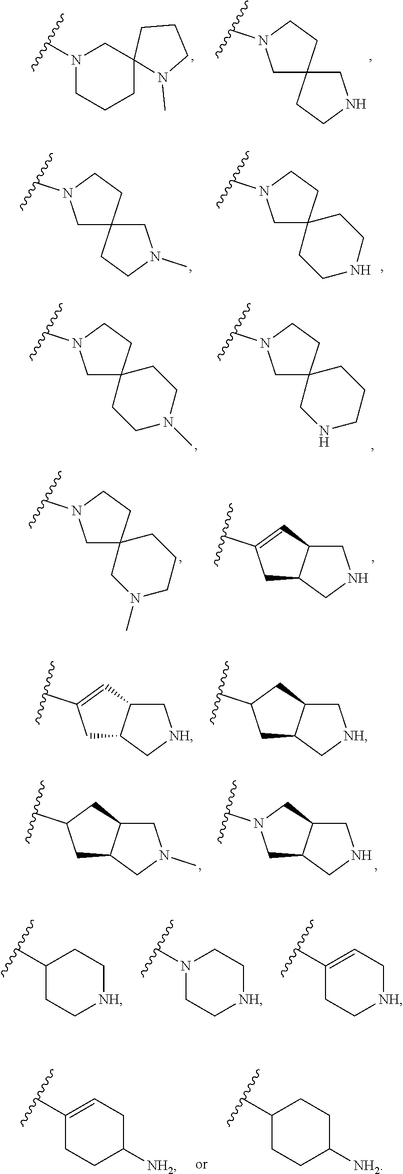

optionally substituted with 1-3 R^{E} (i.e., substituted with 0, 1, 2 or 3 R^{E}), wherein each R^{E} is as defined herein.

In some embodiments, R¹ is

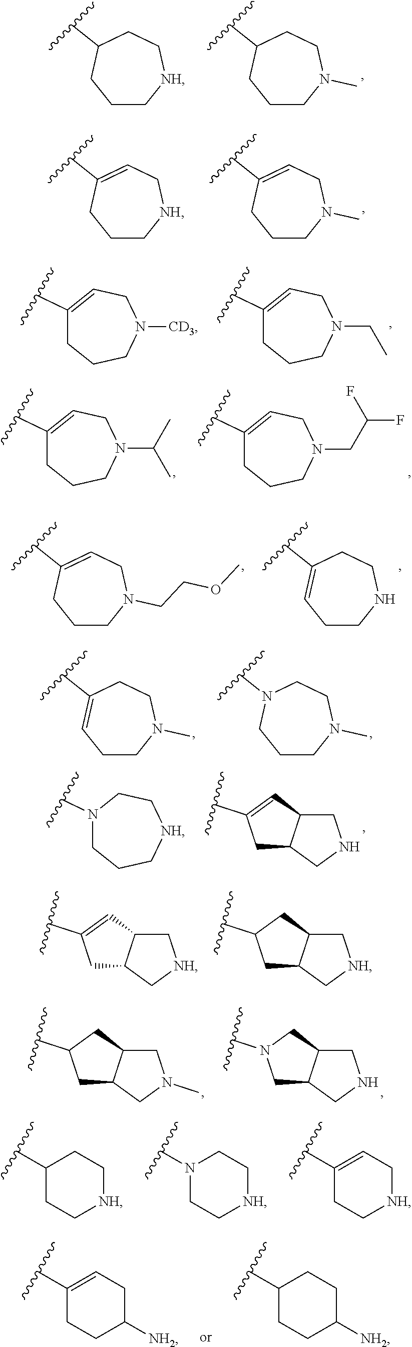

optionally substituted with 1-3 R^{E} (i.e., substituted with 0, 1, 2 or 3 R^{E}), wherein each R^{E} is as defined herein.

In some embodiments, R¹ is

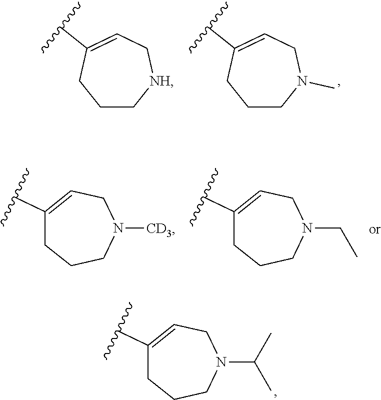

optionally substituted with 1-3 R^{E}, wherein each R^{E} is as defined herein.

In some embodiments, R¹ is

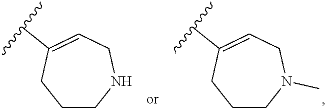

optionally substituted with 1-3 R^{E}, wherein each R^{E} is as defined herein. In some embodiments, R is

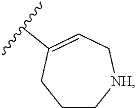 [SMILES: *C1=CCNCCC1]

optionally substituted with 1-3 R^{E}, wherein each R^{E} is as defined herein. In some embodiments, R¹ is

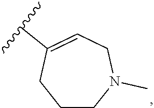 [SMILES: *C1=CCN(C)CCC1]

optionally substituted with 1-3 R^{E}, wherein each R^{E} is as defined herein. In some embodiments, R¹ is

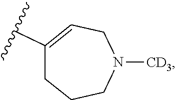 [SMILES: *C1=CCN(C([2H])([2H])[2H])CCC1]

optionally substituted with 1-3 R^{E}, wherein each R^{E} is as defined herein. In some embodiments, R¹ is

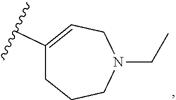 [SMILES: *C1=CCN(CC)CCC1]

optionally substituted with 1-3 R^{E}, wherein each R^{E} is as defined herein. In some embodiments, R¹ is

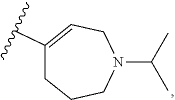 [SMILES: *C1=CCN(C(C)C)CCC1]

optionally substituted with 1-3 R^{E}, wherein each R^{E} is as defined herein.

In some embodiments, R¹ is

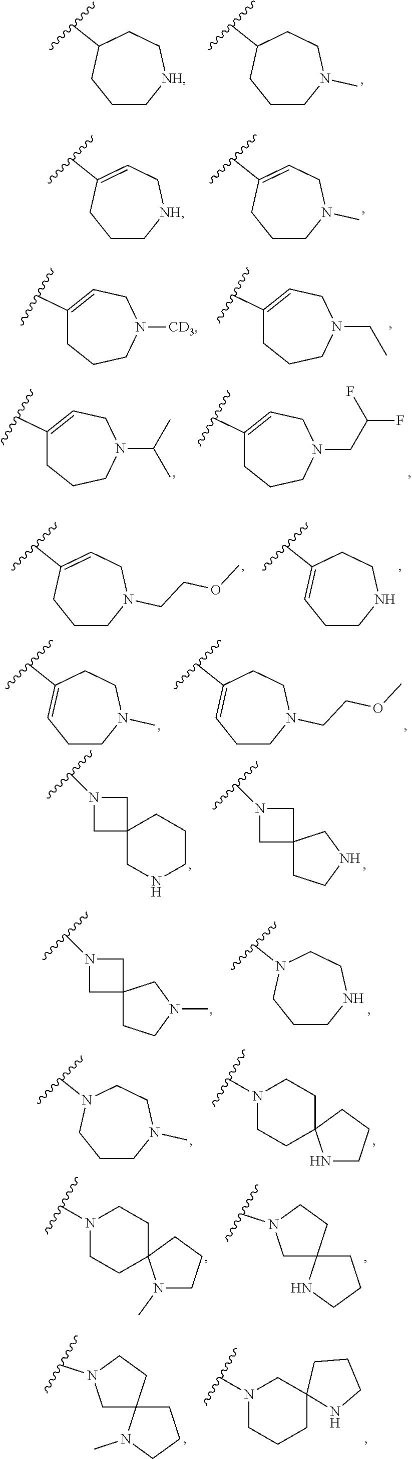

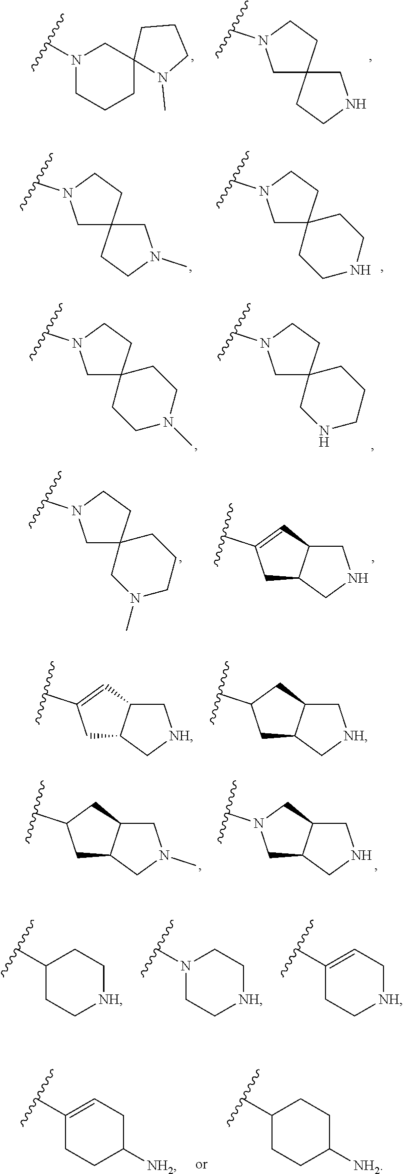

In some embodiments, R¹ is

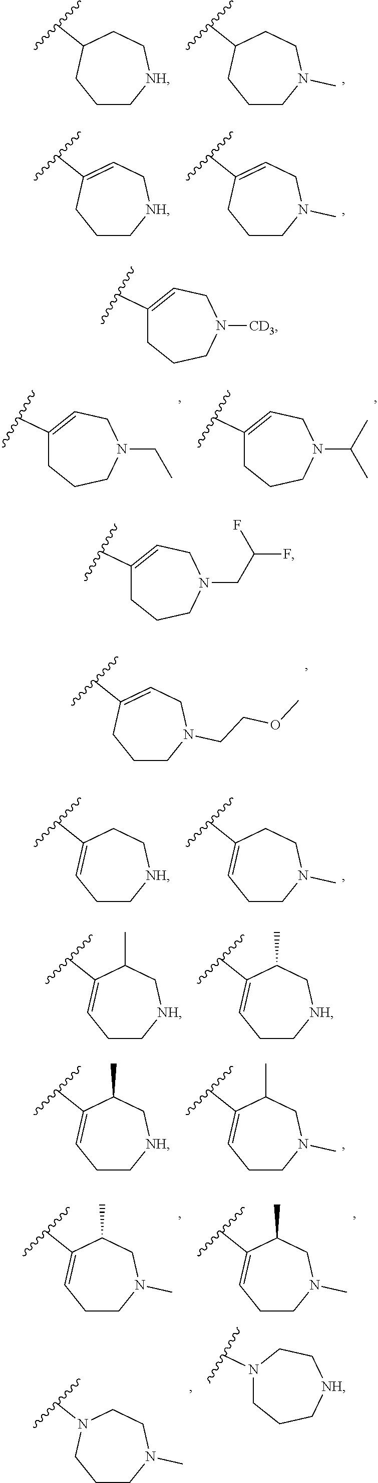

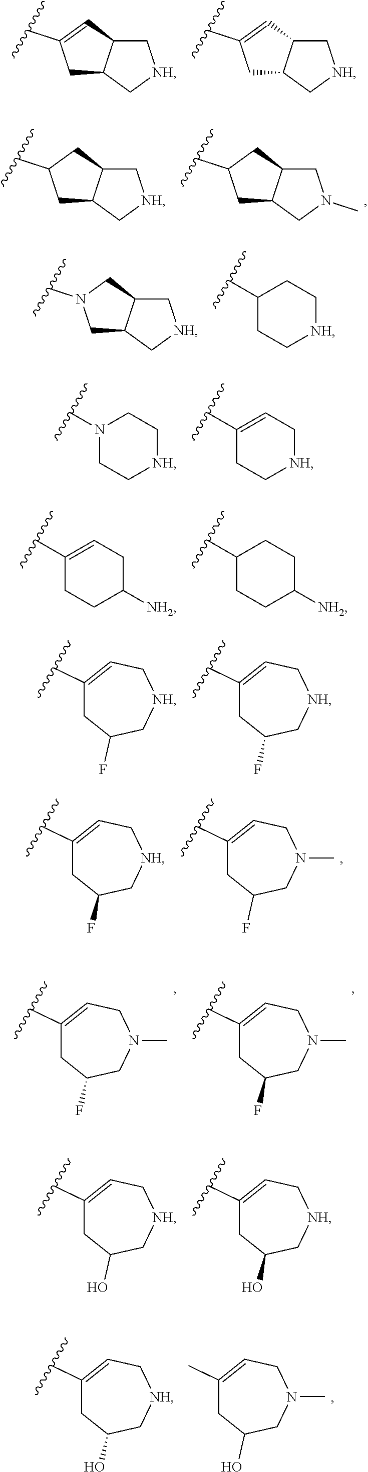

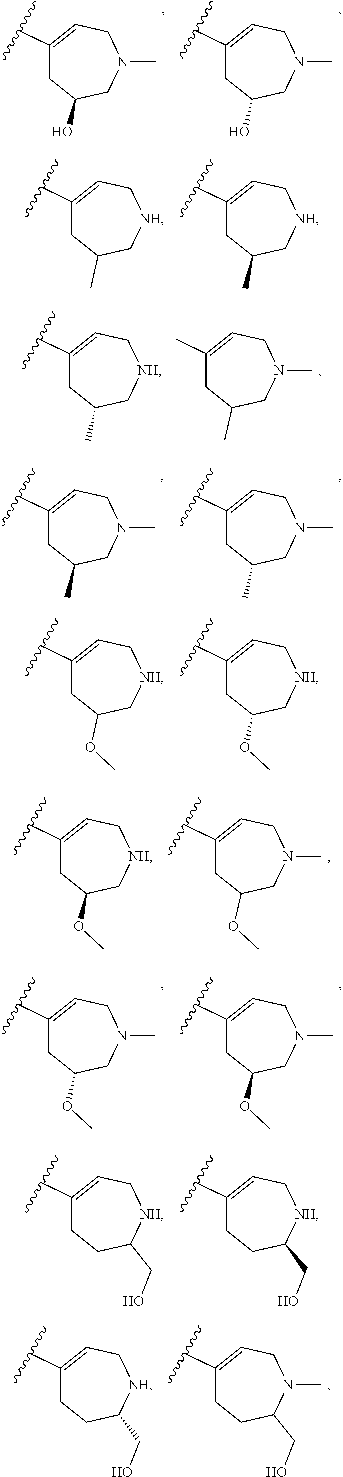

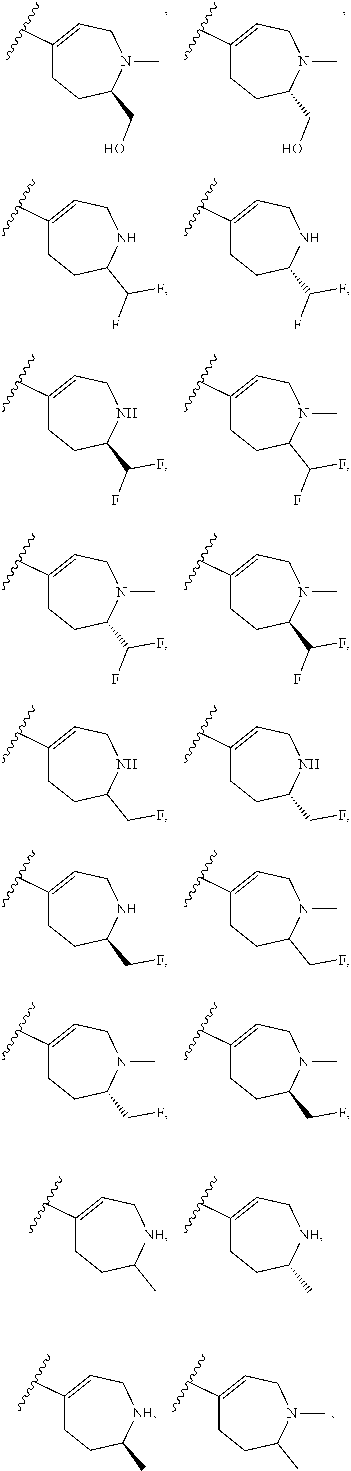

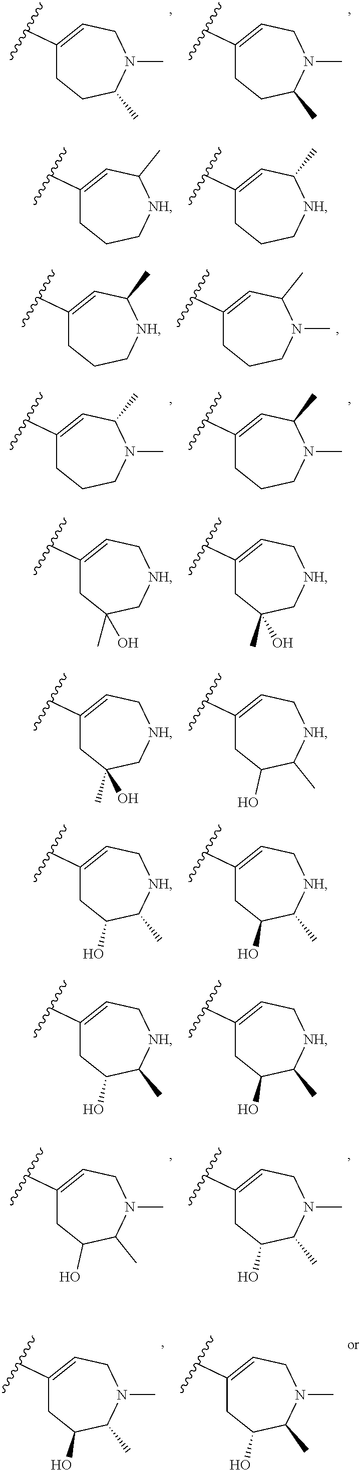

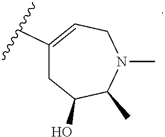 [SMILES: *C1=CCN(C)[C@@H](C)[C@@H](O)C1]

In some embodiments, R¹ is

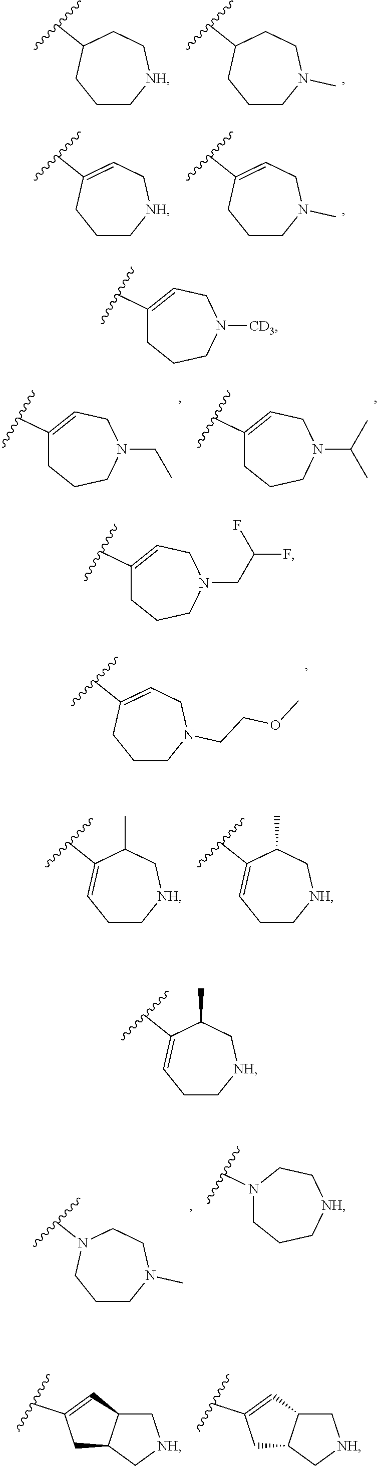

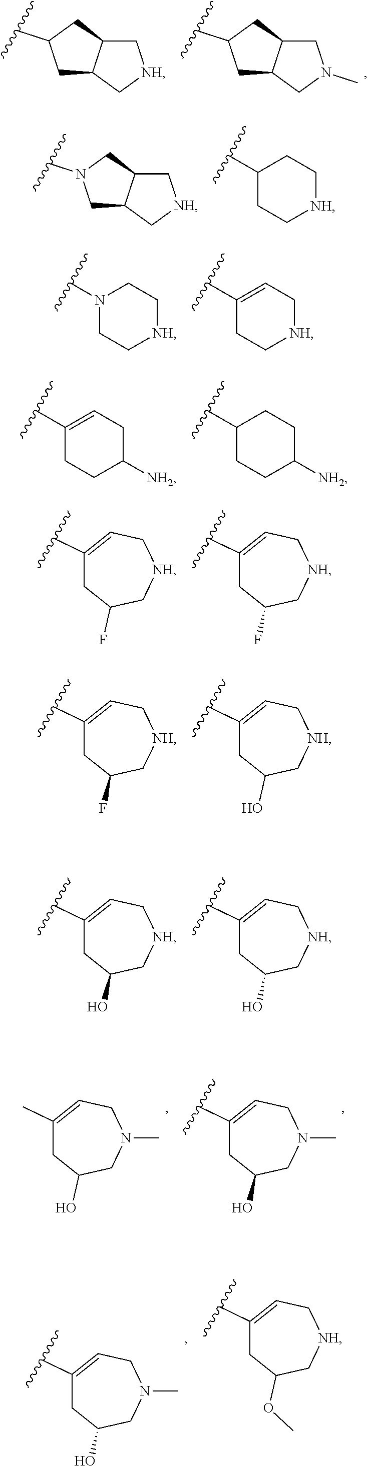

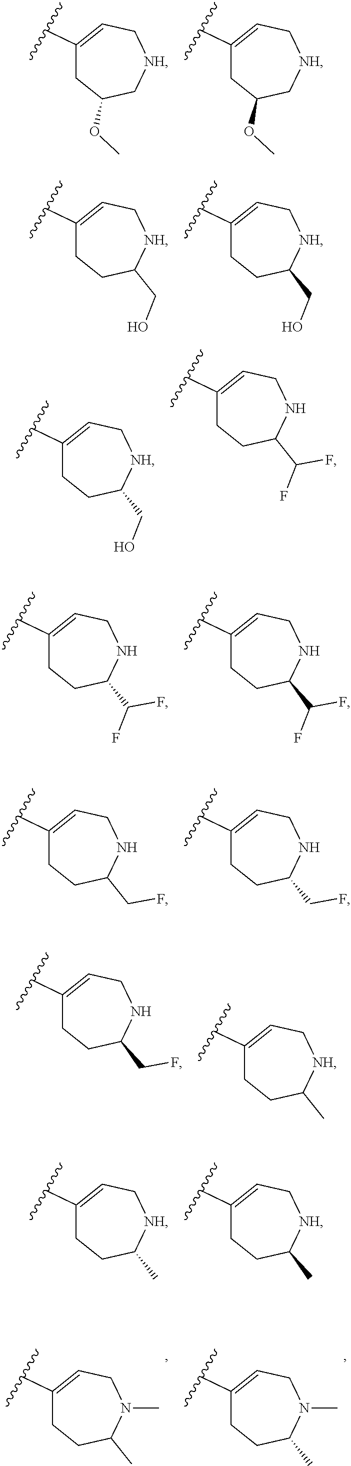

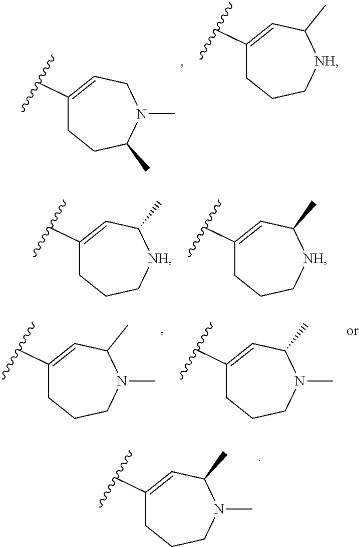

In some embodiments, R¹ is

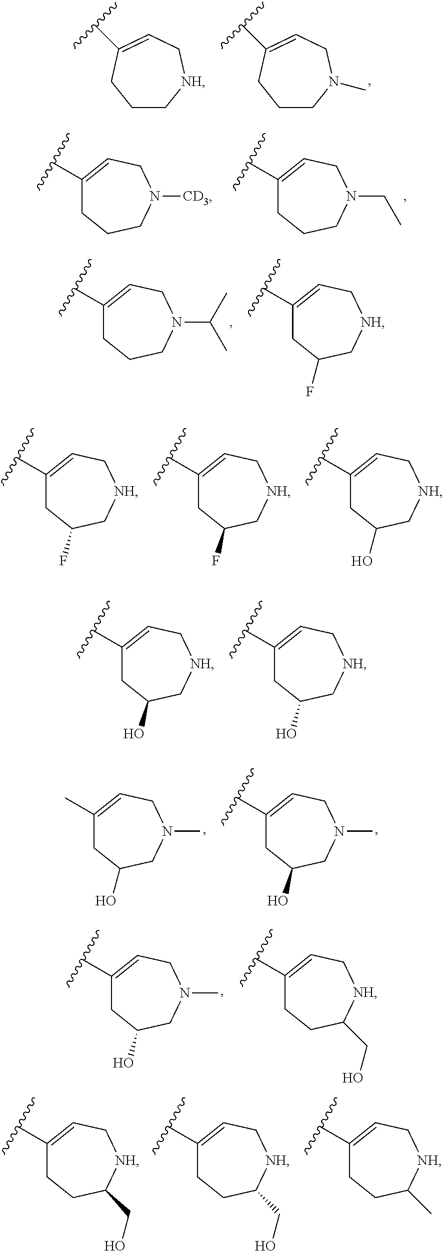

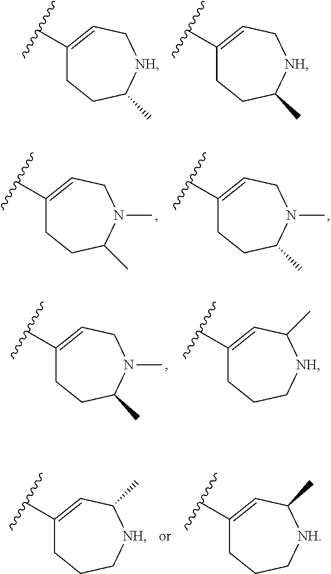

In some embodiments, R¹ is

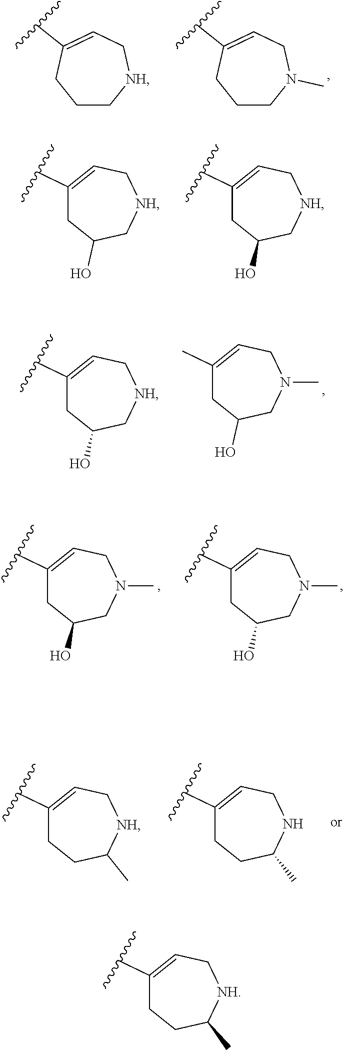

In some embodiments, R¹ is

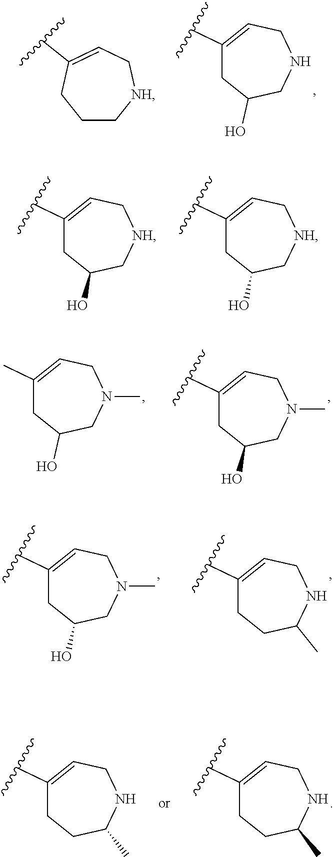

In some embodiments, R¹ is

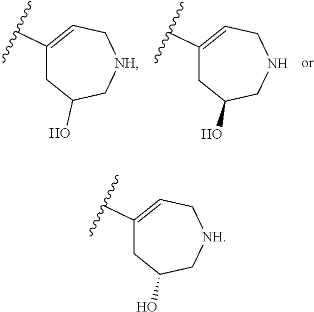 [SMILES: *C1=CCNCC(O)C1 ; *C1=CCNC[C@@H](O)C1 ; *C1=CCNC[C@H](O)C1]

In some embodiments, R¹ is

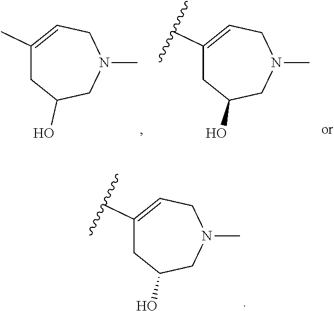

In some embodiments, R¹ is

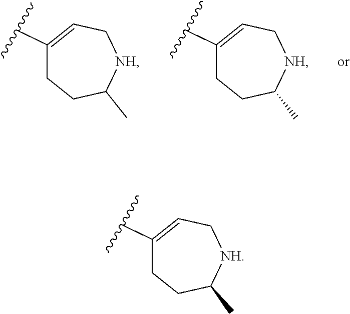

In some embodiments, R¹ is

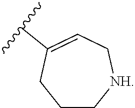 [SMILES: *C1=CCNCCC1]

In some embodiments, R¹ is

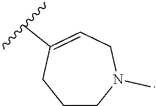 [SMILES: *C1=CCN(C)CCC1]

In some embodiments, R¹ is

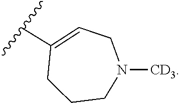 [SMILES: *C1=CCN(C([2H])([2H])[2H])CCC1]

In some embodiments, R¹ is

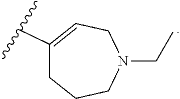 [SMILES: *C1=CCN(CC)CCC1]

In some embodiments, R¹ is

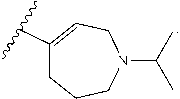 [SMILES: *C1=CCN(C(C)C)CCC1]

As generally defined herein, each R² is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups (i.e., substituted with 0, 1, 2, 3 or 4 groups), independently selected from halogen and OH, and wherein each phenyl, cycloalkyl, or heterocyclyl is optionally substituted with 1-4 R^{E} (i.e., substituted with 0, 1, 2 or 3 R^{E}), wherein R^{C}, R^{D} and R^{E} are as defined herein.

In some embodiments, each R² is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups (i.e., substituted with 0, 1, 2, 3 or 4 groups) independently selected from halogen and OH, and wherein each phenyl, cycloalkyl, or heterocyclyl is optionally substituted with 1-4 R^{E} (i.e., substituted with 0, 1, 2 or 3 R^{E}).

In some embodiments, each R² is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted, and wherein R^{C} and R^{D} are as defined herein.

In some embodiments, each R² is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted, and wherein R^{C} and R^{D} are as defined herein.

In some embodiments, each R² is independently H, C₁-C₆ alkyl, C₁-C₆ haloalkyl, C₁-C₆ heteroalkyl, C₁-C₆ alkoxy, hydroxy, cyano, or halogen.

In some embodiments, each R² is independently H, C₁-C₆ alkyl, C₁-C₆ haloalkyl, C₁-C₆ heteroalkyl, hydroxy, cyano, or halogen.

In some embodiments, R² is C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, C₁-C₆ alkoxy, hydroxy, cyano, CO—R^{C}, NR^{D}₂, or halogen, wherein each alkyl or heteroalkyl is optionally substituted with 1-4 groups (i.e., substituted with 0, 1, 2, 3 or 4 groups) independently selected from halogen and OH, and wherein each phenyl, cycloalkyl, or heterocyclyl is optionally substituted with 1-4 R^{E} (i.e., substituted with 0, 1, 2 or 3 R^{E}).

In some embodiments, R² is C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl or heteroalkyl is optionally substituted with 1-4 groups (i.e., substituted with 0, 1, 2, 3 or 4 groups) independently selected from halogen and OH, and wherein each phenyl, cycloalkyl, or heterocyclyl is optionally substituted with 1-4 R^{E} (i.e., substituted with 0, 1, 2 or 3 R^{E}).

In some embodiments, R² is H, C₁-C₆ alkyl, C₁-C₆ haloalkyl, C₁-C₆ heteroalkyl, C₁-C₆ alkoxy, hydroxy, cyano, or halogen.

In some embodiments, R² is C₁-C₆ alkyl, C₁-C₆ haloalkyl, C₁-C₆ heteroalkyl, C₁-C₆ alkoxy, hydroxy, cyano, or halogen.

In some embodiments, R² is H, C₁-C₆ alkyl or halogen. In some embodiments, R² is C₁-C₆ alkyl or halogen.

In some embodiments, R² is halogen.

In some embodiments, R² is C₁-C₆ alkyl.

In some embodiments, R² is selected from H, Me, F and Cl.

In some embodiments, R² is selected from Me, F and Cl.

In some embodiments, R² is selected from F and Cl.

In some embodiments, R² is H. In some embodiments, R² is Me. In some embodiments, R² is Cl. In some embodiments, R² is F.

As generally defined herein, each R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups (i.e., substituted with 0, 1, 2, 3 or 4 groups) independently selected from halogen and OH, and wherein each phenyl, cycloalkyl, or heterocyclyl is optionally substituted with 1-4 R^{E} (i.e., substituted with 0, 1, 2 or 3 R^{E}), wherein R^{C}, R^{D} and R^{E} are as defined herein.

In some embodiments, each R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups (i.e., substituted with 0, 1, 2, 3 or 4 groups) independently selected from halogen and OH, and wherein each phenyl, cycloalkyl, or heterocyclyl is optionally substituted with 1-4 R^{E} (i.e., substituted with 0, 1, 2 or 3 R^{E}).

In some embodiments, each R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted, and wherein R^{C} and R^{D} are as defined herein.

In some embodiments, each R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted, and wherein R^{C} and R^{D} are as defined herein.

In some embodiments, each R³ is independently H, C₁-C₆ alkyl, C₁-C₆ haloalkyl, C₁-C₆ heteroalkyl, C₁-C₆ alkoxy, hydroxy, cyano, or halogen.

In some embodiments, each R³ is independently H, C₁-C₆ alkyl, C₁-C₆ haloalkyl, C₁-C₆ heteroalkyl, hydroxy, cyano, or halogen.

In some embodiments, each R³ is H.

In some embodiments, each R² and R³ is independently H, C₁-C₆ alkyl, C₁-C₆ haloalkyl, C₁-C₆ heteroalkyl, hydroxy, cyano, or halogen.

In some embodiments, each of R² and R³ is H.

As generally defined herein, R⁴ is H, C₁-C₆ alkyl or C₁-C₆ heteroalkyl.

In some embodiments, R⁴ is H or C₁-C₆ alkyl.

In some embodiments, R⁴ is H or Me.

In some embodiments, R⁴ is H.

As generally defined herein, each R⁵ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E} (i.e., substituted with 0, 1, 2 or 3 R^{E}), wherein R^{E} is as defined herein.

In some embodiments, each R⁵ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted

In some embodiments, R⁵ is H.

As generally defined herein, each R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E} (i.e., substituted with 0, 1, 2 or 3 R^{E}), wherein R^{E} is as defined herein.

In some embodiments, each R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted

In some embodiments, R⁵ and R⁶ are taken together with the nitrogen to which they are attached to form a 3-7 membered heterocycle with 0-2 additional ring heteroatoms selected from O, S, and N, and wherein the heterocycle is optionally substituted.

In some embodiments, R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E} (i.e., substituted with 0, 1, 2 or 3 R^{E}), wherein R^{E} is as defined herein.

In some embodiments, R⁶ is selected from H, C₁-C₆ alkyl, C₁-C₆ heteroalkyl, phenyl, C₃-C₇ cycloalkyl, C₃-C₁₀ heterocyclyl, C₁-C₆ alkylene-phenyl, C₁-C₆ alkylene-C₃-C₇ cycloalkyl, and C₁-C₆ alkylene-C₃-C₁₀ heterocyclyl, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E} (i.e., substituted with 0, 1, 2 or 3 R^{E}).

In some embodiments, R⁶ is selected from C₁-C₆ alkyl, C₁-C₆ heteroalkyl, C₃-C₇ cycloalkyl, C₃-C₁₀ heterocyclyl, C₁-C₆ alkylene-C₃-C₇ cycloalkyl, and C₁-C₆ alkylene-C₃-C₁₀ heterocyclyl, wherein each alkyl or heteroalkyl is optionally substituted with 1-5 substituents (i.e., substituted by 0, 1, 2, 3, 4 or 5 substituents) independently selected from deuterium, halogen and OH, and wherein each cycloalkyl, or heterocyclyl is optionally substituted with 1-4 R^{E}.

In some embodiments, R⁶ is selected from C₁-C₆ alkyl and C₁-C₆ heteroalkyl, wherein each alkyl or heteroalkyl is optionally substituted with 1-5 substituents (i.e., substituted by 0, 1, 2, 3, 4 or 5 substituents) independently selected from deuterium, halogen and OH.

In some embodiments, R⁶ is selected from C₁-C₆ alkyl and C₁-C₆ heteroalkyl, wherein each alkyl or heteroalkyl is optionally substituted with 1-5 instances of deuterium (i.e., substituted by 0, 1, 2, 3, 4 or 5 deuteriums).

In some embodiments, R⁶ is C₁-C₆ alkyl, optionally substituted with one or more deuteriums. In some embodiments, R⁶ is C₁-C₆ alkyl.

In some embodiments, R⁶ is C₁-C₆ alkyl optionally substituted with 1-5 instances of deuterium (i.e., substituted by 0, 1, 2, 3, 4 or 5 deuteriums).

In some embodiments, R⁶ is selected from Me, CD₃, Et, CH₂CD₃, CH₂CH₂OMe, CH₂CH₂CH₂OMe, CH₂CF₃, cyclobutyl, cyclopentyl, cyclohexyl, oxetanyl, CH₂-tetrahydropyranyl, CH₂-tetrahydrofuran-2-yl, N-iPr-piperidin-4-yl.

In some embodiments, R⁶ is selected from Me, CD₃, Et, CH₂CD₃, CH₂CH₂OMe and CH₂CH₂CH₂OMe.

In other embodiments, R⁶ is C₃-C₇ cycloalkyl or heterocyclyl, each optionally substituted with 1-4 R^{E} (i.e., substituted with 0, 1, 2 or 3 R^{E}).

In some embodiments, R⁶ is selected from Me and CD₃. In some embodiments, R⁶ is Me. In some embodiments, R⁶ is CD₃.

In some embodiments, R⁵ and R⁶ are each C₁-C₆ alkyl, optionally substituted with one or more deuteriums. In some embodiments, R⁵ and R⁶ are each C₁-C₆ alkyl.

In some embodiments, R⁵ is H and R⁶ is not H.

In some embodiments, R⁵ and R⁶ are each Me.

As generally defined herein, each R⁷ is independently selected from H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E}(i.e., substituted with 0, 1, 2 or 3 R^{E}) and wherein R^{C}, R^{D} and R^{E} are as defined herein.

In some embodiments, each R⁷ is independently selected from H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E} (i.e., substituted with 0, 1, 2 or 3 R^{E}).

In some embodiments, each R⁷ is independently selected from H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, and halogen, wherein each alkyl, heteroalkyl, phenyl, cycloalkyl, or heterocyclyl is optionally substituted.

In some embodiments, each R⁷ is independently selected from H, C₁-C₆ alkyl, C₁-C₆ heteroalkyl, phenyl, cycloalkyl, heterocyclyl, hydroxy, cyano, CO—R^{C}, NR^{D}₂, and halogen, wherein each alkyl, heteroalkyl, phenyl, cycloalkyl, or heterocyclyl is optionally substituted.

In some embodiments, each R⁷ is independently selected from H, C₁-C₆ alkyl, C₁-C₆ alkoxy, C₁-C₆ haloalkyl, C₁-C₆ heteroalkyl, hydroxy, cyano, and halogen, each alkyl or heteroalkyl optionally substituted with 1-5 deuteriums (i.e., substituted by 0, 1, 2, 3, 4 or 5 deuteriums).

In some embodiments, each R⁷ is independently selected from H, C₁-C₆ alkyl, C₁-C₆ haloalkyl, C₁-C₆ heteroalkyl, hydroxy, cyano, and halogen, each alkyl or heteroalkyl optionally substituted with 1-5 deuteriums (i.e., substituted by 0, 1, 2, 3, 4 or 5 deuteriums).

In some embodiments, R⁷ is selected from H, C₁-C₆ alkyl, C₁-C₆ alkoxy, C₁-C₆ heteroalkyl, phenyl, C₃-C₇ cycloalkyl, C₃-C₁₀ heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E}(i.e., substituted with 0, 1, 2 or 3 R^{E}).

In some embodiments, R⁷ is selected from H, C₁-C₆ alkyl, C₁-C₆ alkoxy, and halogen, wherein each alkyl is optionally substituted with 1-5 instances of deuterium (i.e., substituted by 0, 1, 2, 3, 4 or 5 deuteriums).

In some embodiments, R⁷ is selected from H, F, Cl, Me, Et and OMe.

In some embodiments, R⁷ is selected from H, F and Me. In some embodiments, R⁷ is H or F. In some embodiments, R⁷ is H. In some embodiments, R⁷ is F.

As generally defined herein, each R⁸ is independently selected from H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E}(i.e., substituted with 0, 1, 2 or 3 R^{E}) and wherein R^{C}, R^{D} and R^{E} are as defined herein.

In some embodiments, each R⁸ is independently selected from H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E} (i.e., substituted with 0, 1, 2 or 3 R^{E}).

In some embodiments, each R⁸ is independently selected from H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, and halogen, wherein each alkyl, heteroalkyl, phenyl, cycloalkyl, or heterocyclyl is optionally substituted.

In some embodiments, each R⁸ is independently selected from H, C₁-C₆ alkyl, C₁-C₆ heteroalkyl, phenyl, cycloalkyl, heterocyclyl, hydroxy, cyano, CO—R^{C}, NR^{D}₂, and halogen, wherein each alkyl, heteroalkyl, phenyl, cycloalkyl, or heterocyclyl is optionally substituted.

In some embodiments, each R⁸ is independently selected from H, C₁-C₆ alkyl, C₁-C₆ alkoxy, C₁-C₆ haloalkyl, C₁-C₆ heteroalkyl, hydroxy, cyano, and halogen, each alkyl or heteroalkyl optionally substituted with 1-5 deuteriums (i.e., substituted by 0, 1, 2, 3, 4 or 5 deuteriums).

In some embodiments, each R⁸ is independently selected from H, C₁-C₆ alkyl, C₁-C₆ haloalkyl, C₁-C₆ heteroalkyl, hydroxy, cyano, and halogen, each alkyl or heteroalkyl optionally substituted with 1-5 deuteriums (i.e., substituted by 0, 1, 2, 3, 4 or 5 deuteriums).

In some embodiments, R⁸ is selected from H, C₁-C₆ alkyl, C₁-C₆ alkoxy, C₁-C₆ heteroalkyl, phenyl, C₃-C₇ cycloalkyl, C₃-C₁₀ heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents (i.e., substituted by 0, 1, 2, 3, 4 or 5 substituents) independently selected from deuterium, halogen and OH, and wherein each phenyl, cycloalkyl, or heterocyclyl is optionally substituted with 1-4 R^{E}(i.e., substituted with 0, 1, 2 or 3 R^{E}).

In some embodiments, R⁸ is C₁-C₆ alkyl, C₃-C₇ cycloalkyl, heterocyclyl, wherein each alkyl is optionally substituted with 1-4 substituents (i.e., substituted with 0, 1, 2, 3 or 4 substituents) independently selected from deuterium, halogen and OH, and wherein each cycloalkyl or heterocyclyl is optionally substituted with 1-4 R^{E} (i.e., substituted with 0, 1, 2 or 3 R^{E}).

In some embodiments, R⁸ is C₁-C₆ alkyl, C₃-C₇ cycloalkyl, heterocyclyl, wherein each alkyl is optionally substituted with 1-4 substituents (i.e., substituted with 0, 1, 2, 3 or 4 substituents) independently selected from halogen and OH, and wherein each cycloalkyl or heterocyclyl is optionally substituted with 1-4 R^{E} (i.e., substituted with 0, 1, 2 or 3 R^{E}).

In some embodiments, R⁸ is C₁-C₆ alkyl, optionally substituted with 1-5 deuteriums (i.e., substituted by 0, 1, 2, 3, 4 or 5 deuteriums). In some embodiments, R⁸ is C₁-C₆ alkyl.

In some embodiments, R⁸ is selected from methyl, ethyl, CH₂D, iPr, cyclopropyl, cyclohexyl and CH₂CF₃.

In some embodiments, R⁸ is selected from methyl and CH₂D. In some embodiments, R⁸ is Me. In some embodiments, R⁸ is CH₂D.

As generally defined herein, each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 R^{E} (i.e., substituted with 0, 1, 2 or 3 R^{E}), wherein R^{C}, R^{D} and R^{E} are as defined herein; or R⁹ and R¹⁰ can be taken together with the carbon to which they are attached to form CO.

In some embodiments, each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 R^{E} (i.e., substituted with 0, 1, 2 or 3 R^{E}), wherein R^{C}, R^{D} and R^{E} are as defined herein.

In some embodiments, each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted.

In some embodiments, each R⁹ and R¹⁰ is independently H, C₁-C₆ alkyl, C₁-C₆ haloalkyl, C₁-C₆ heteroalkyl, hydroxy, cyano, or halogen.

In some embodiments, each R⁹ and R¹⁰ is independently H, C₁-C₆ alkyl, or halogen, or R⁹ and R¹⁰ together with the carbon to which they are attached form CO.

In some embodiments, each R⁹ and R¹⁰ is independently H, C₁-C₆ alkyl, or halogen.

In some embodiments, each R⁹ and R¹⁰ is independently H, C₁-C₆ alkyl, or halogen.

In some embodiments, each R⁹ and R¹⁰ is independently H, Me or F.

In some embodiments, each R⁹ and R¹⁰ is independently H. As generally defined herein, each R¹¹ is independently selected from H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, and halogen, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 R^{E} (i.e., substituted with 0, 1, 2 or 3 R^{E}); wherein R^{C}, R^{D} and R^{E} are as defined herein; or two R¹¹ can be taken together with the carbon to which they are attached to form CO or a spirofused C₃-C₇ cycloalkyl.

In some embodiments, each R¹¹ is independently selected from H, C₁-C₆ alkyl, C₁-C₆ heteroalkyl, phenyl, cycloalkyl, heterocyclyl, hydroxy, cyano, CO—R^{C}, NR^{D}₂, and halogen, wherein each alkyl, heteroalkyl, phenyl, cycloalkyl, or heterocyclyl is optionally substituted; or two R¹¹ can be taken together with the carbon to which they are attached to form CO or a spirofused C₃-C₇ cycloalkyl.

In some embodiments, each R¹¹ is independently selected from H, C₁-C₆ alkyl, hydroxy, and halogen, wherein each alkyl is optionally substituted with 1-4 substituents (i.e., substituted with 0, 1, 2, 3 or 4 substituents) independently selected from halogen and OH; or two R¹¹ are taken together with the carbon to which they are attached to form CO or a spirofused C₃-C₇ cycloalkyl.

In some embodiments, each R¹¹ is independently selected from H, C₁-C₆ alkyl, C₁-C₆ haloalkyl, C₁-C₆ heteroalkyl, hydroxy, cyano, or halogen.

In some embodiments, each R¹¹ is independently selected from H, C₁-C₆ alkyl, hydroxy, and halogen, or two R¹¹ are taken together with the carbon to which they are attached to form a spirofused C₃-C₇ cycloalkyl.

In some embodiments, each R¹¹ is independently selected from H, Me, hydroxy, and F, or two R¹¹ are taken together with the carbon to which they are attached to form a spirofused cyclopropyl.

In some embodiments, each R¹¹ is independently selected from H and Me.

In some embodiments, each R¹¹ is independently selected from hydroxy and Me.

In some embodiments, each R¹¹ is independently H. In some embodiments, each R¹¹ is independently Me. In some embodiments, each R¹¹ is independently F.

In some embodiments, two R¹¹ are taken together with the carbon to which they are attached to form a spirofused cyclopropyl.

As generally defined herein, each R¹² is independently selected from H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 R^{E} (i.e., substituted with 0, 1, 2 or 3 R^{E}); or two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E} (i.e., substituted with 0, 1, 2 or 3 R^{E}), wherein R^{C}, R^{D} and R^{E} are as defined herein.

In some embodiments, each R¹² is independently selected from H, C₁-C₆ alkyl, C₁-C₆ heteroalkyl, phenyl, C₃-C₇ cycloalkyl, and heterocyclyl, wherein each alkyl, heteroalkyl, phenyl, cycloalkyl, or heterocyclyl is optionally substituted or two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted.

In some embodiments, each R¹² is independently selected from H, C₁-C₆ alkyl, C₁-C₆ haloalkyl, and C₁-C₆ heteroalkyl. In some embodiments, each R¹² is independently selected from H, C₁-C₆ alkylene-phenyl and C₁-C₆ alkyl.

In some embodiments, each R¹² is independently H or C₁-C₆ alkyl. In some embodiments, each R¹² is H.

As generally defined herein, each R¹³ is independently selected from H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, C₁-C₆ alkylene- heterocyclyl, hydroxy, cyano, CO—R^{C}, NR^{D}₂, and halogen, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 R^{E} (i.e., substituted with 0, 1, 2 or 3 R^{E}); wherein R^{C}, R^{D} and R^{E} are as defined herein; or two R¹¹ can be taken together with the carbon to which they are attached to form CO or a spirofused C₃-C₇ cycloalkyl.

In some embodiments, each R¹³ is independently selected from H, C₁-C₆ alkyl, C₁-C₆ heteroalkyl, phenyl, cycloalkyl, heterocyclyl, hydroxy, cyano, CO—R^{C}, NR^{D}₂, and halogen, wherein each alkyl, heteroalkyl, phenyl, cycloalkyl, or heterocyclyl is optionally substituted; or two R¹¹ can be taken together with the carbon to which they are attached to form CO or a spirofused C₃-C₇ cycloalkyl.

In some embodiments, each R¹³ is independently selected from H, C₁-C₆ alkyl, C₁-C₆ heteroalkyl, hydroxy, cyano, and halogen, wherein each alkyl or heteroalkyl is optionally substituted with 1-4 substituents (i.e., substituted with 0, 1, 2, 3 or 4 substituents) independently selected from halogen and OH, or two R¹³ are taken together with the carbon to which they are attached to form CO or a spirofused C₃-C₇ cycloalkyl.

In some embodiments, each R¹³ is independently selected from H, C₁-C₆ alkyl, hydroxy, and halogen, or two R¹³ are taken together with the carbon to which they are attached to form a spirofused C₃-C₇ cycloalkyl.

In some embodiments, each R¹³ is independently selected from H, C₁-C₆ alkyl, C₁-C₆ haloalkyl, C₁-C₆ heteroalkyl, hydroxy, cyano, or halogen.

In some embodiments, each R¹³ is independently selected from H, Me, hydroxy, and F, or two R¹³ are taken together with the carbon to which they are attached to form a spirofused cyclopropyl.

In some embodiments, each R¹³ is independently H.

As generally defined herein, each R^{c} is independently selected from H, OH, N(R¹²)₂, C₁-C₆ alkyl, C₁-C₆ alkoxy, C₁-C₆ heteroalkyl, phenyl, C₃-C₇ cycloalkyl, and heterocyclyl, wherein each alkyl, heteroalkyl, phenyl, cycloalkyl, or heterocyclyl is optionally substituted.

In some embodiments, each R^{c} is independently selected from H, OH, N(R¹²)₂, C₁-C₆ alkyl, C₁-C₆ alkoxy, C₁-C₆ heteroalkyl, phenyl, C₃-C₇ cycloalkyl, and heterocyclyl, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 substituents (i.e., substituted with 0, 1, 2, 3 or 4 substituents) independently selected from halogen, C₁-C₆ alkyl, C₁-C₆ haloalkyl, C₁-C₆ alkoxy, and OH.

In some embodiments, each R^{c} is independently H, OH, NR¹²₂, C₁-C₆ alkyl, or C₁-C₆ alkoxy.

As generally defined herein, each R^{D} is independently selected from H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, and heterocyclyl, wherein each alkyl, heteroalkyl, phenyl, cycloalkyl, or heterocyclyl is optionally substituted; or two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; wherein w is 0, 1, or 2.

In some embodiments, each R^{D} is independently selected from R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, and heterocyclyl, wherein each alkyl or heteroalkyl is optionally substituted with 1-4 substituents (i.e., substituted with 0, 1, 2, 3 or 4 substituents) independently selected from halogen and OH, and wherein each phenyl, cycloalkyl, or heterocyclyl is optionally substituted with 1-4 substituents (i.e., substituted with 0, 1, 2, 3 or 4 substituents) independently selected from halogen, C₁-C₆ alkyl, C₁-C₆ haloalkyl, C₁-C₆ alkoxy, and OH; or two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents (i.e., substituted with 0, 1, 2, 3 or 4 substituents) independently selected from halogen, C₁-C₆ alkyl, C₁-C₆ haloalkyl, C₁-C₆ alkoxy, and OH, wherein w is 0, 1, or 2.

In some embodiments, each R^{D} is independently H or C₁-C₆ alkyl.

In certain embodiments, provided herein is a compound of Formula (II), or a pharmaceutically acceptable salt thereof, wherein the compound is of Formula (IIa)

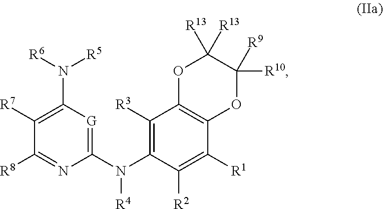

wherein the variables are as defined herein.

In certain embodiments, provided herein is a compound of Formula (II), or a pharmaceutically acceptable salt thereof, wherein the compound is of Formula (IIb)

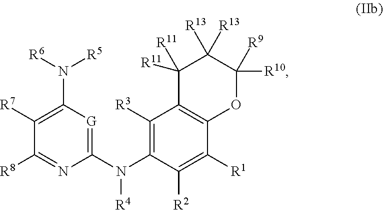

wherein the variables are as defined herein.

In certain embodiments, provided herein is a compound of Formula (II), or a pharmaceutically acceptable salt thereof, wherein the compound is of Formula (IIc)

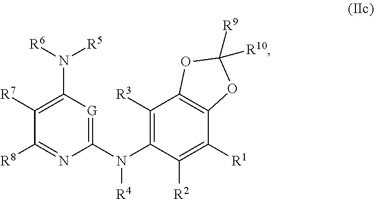

wherein the variables are as defined herein.

In certain embodiments, provided herein is a compound of Formula (II), or a pharmaceutically acceptable salt thereof, wherein the compound is of Formula (IId)

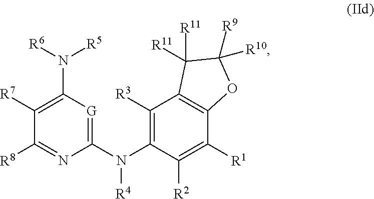

wherein the variables are as defined herein.

In certain embodiments, provided herein is a compound of Formula (II), or a pharmaceutically acceptable salt thereof, wherein the compound is of Formula (IIIa)

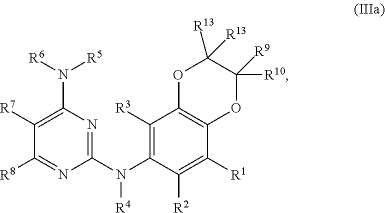

wherein the variables are as defined herein.

In certain embodiments, provided herein is a compound of Formula (II), or a pharmaceutically acceptable salt thereof, wherein the compound is of Formula (IIIb)

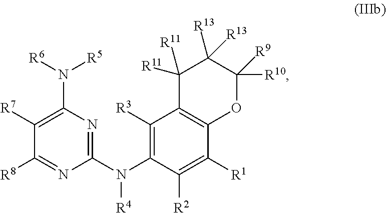

wherein the variables are as defined herein.

In certain embodiments, provided herein is a compound of Formula (II), or a pharmaceutically acceptable salt thereof, wherein the compound is of Formula (IIc)

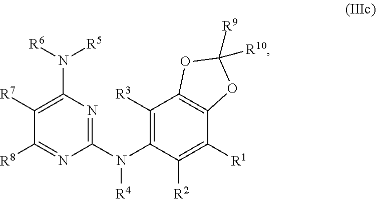

wherein the variables are as defined herein.

In certain embodiments, provided herein is a compound of Formula (II), or a pharmaceutically acceptable salt thereof, wherein the compound is of Formula (IIId)

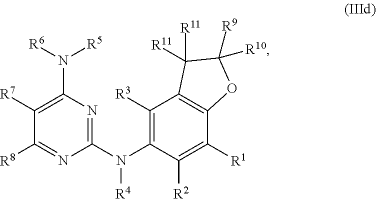

wherein the variables are as defined herein.

In certain embodiments, provided herein is a compound of Formula (IVa), or a pharmaceutically acceptable salt thereof,

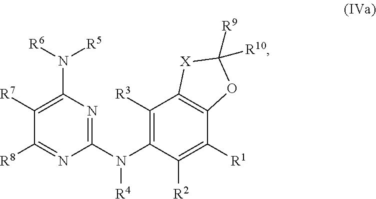

wherein the variables are as defined herein.

In certain embodiments, provided herein is a compound of Formula (IVb), or a pharmaceutically acceptable salt thereof,

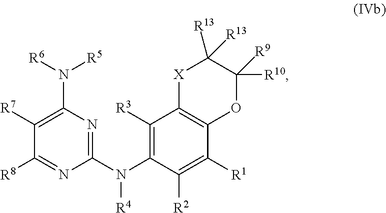

wherein the variables are as defined herein.

In certain embodiments, provided herein is a compound of Formula (IIa-1), or a pharmaceutically acceptable salt thereof,

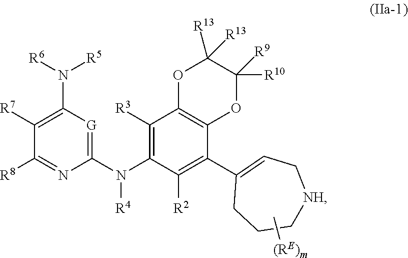

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IIb-1), or a pharmaceutically acceptable salt thereof,

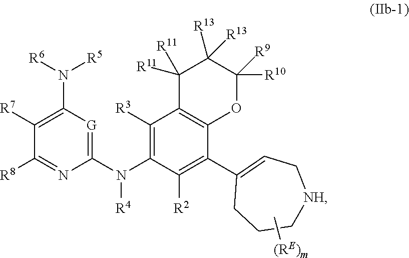

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IIc-1), or a pharmaceutically acceptable salt thereof,

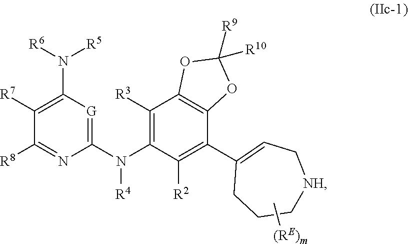

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IId-1), or a pharmaceutically acceptable salt thereof,

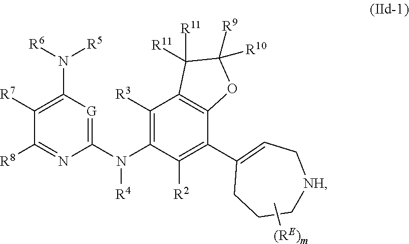

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IIIa-1), or a pharmaceutically acceptable salt thereof,

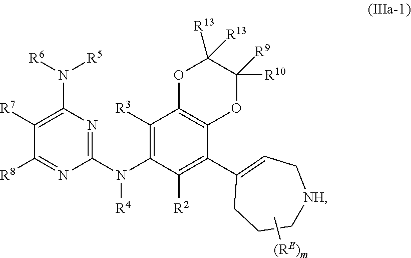

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IIIb-1), or a pharmaceutically acceptable salt thereof,

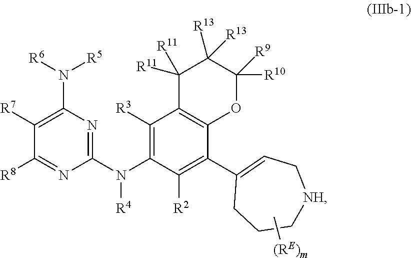

wherein m is 0, 1, 2, 3 or 4 and the remaining variables are as defined herein. For clarity, individual instances of R^{E} can be attached to any of the carbon atoms or to the nitrogen atom.

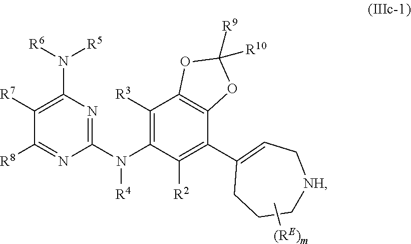

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IIId-1), or a pharmaceutically acceptable salt thereof,

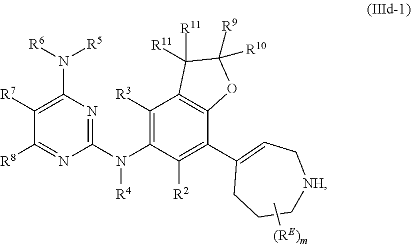

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IVa-1), or a pharmaceutically acceptable salt thereof,

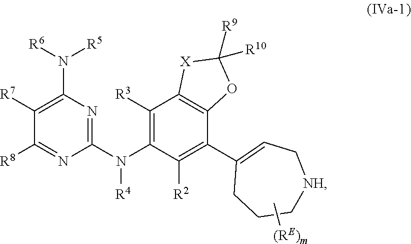

wherein m is 0, 1, 2, 3 or 4 and the remaining variables are as defined herein. For clarity, individual instances of R^{E} can be attached to any of the carbon atoms or to the nitrogen atom.

In certain embodiments, provided herein is a compound of Formula (IVb-1), or a pharmaceutically acceptable salt thereof,

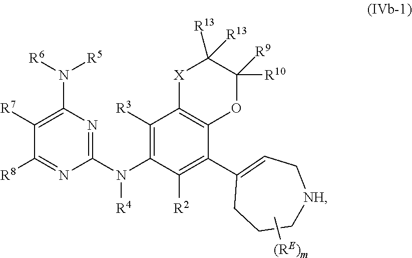

wherein m is 0, 1, 2, 3 or 4 and the remaining variables are as defined herein. For clarity, individual instances of R^{E} can be attached to any of the carbon atoms or to the nitrogen atom.

In some embodiments, m is 0. In some embodiments, m is 1, 2 or 3. In some embodiments, m is 1 or 2. In some embodiments, m is 1. In some embodiments, m is 2. In some embodiments, m is 3. In some embodiments, m is 4.

In some embodiments, the compound is of Formula (I) wherein A is an optionally substituted 5-membered oxygen-containing heterocyclic ring. In some embodiments, the compound is selected from the group consisting of:

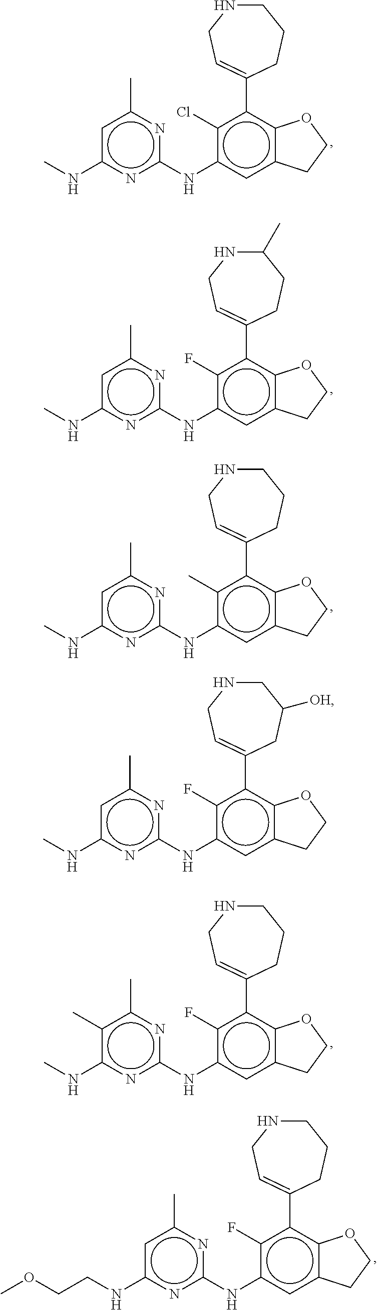

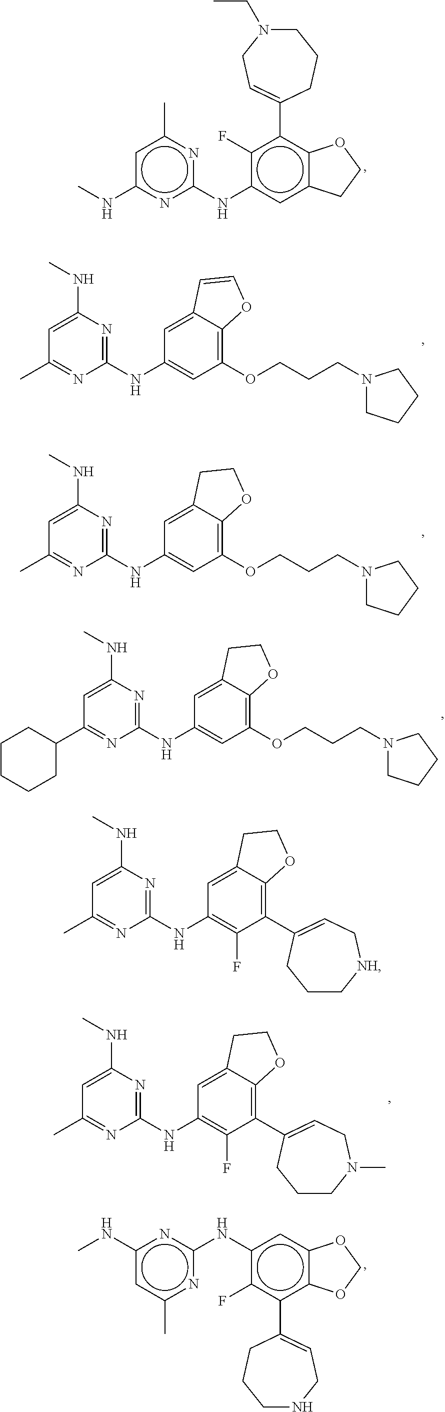

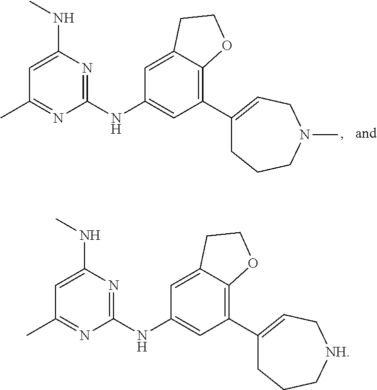

In some embodiments, the compound is of Formula (I) wherein A is an optionally substituted 6-membered oxygen-containing heterocyclic ring. In some embodiments, the compound is selected from the group consisting of:

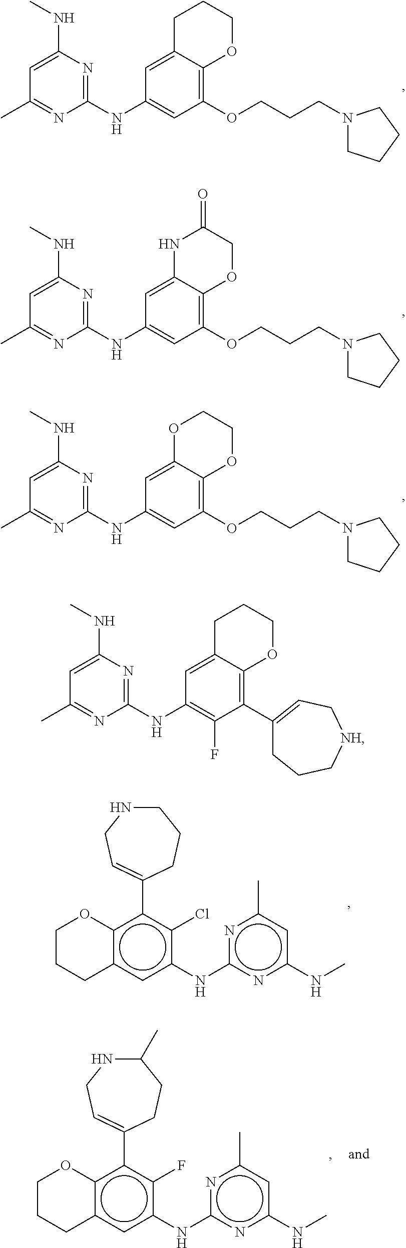

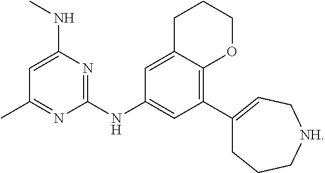 [SMILES: CNc1cc(C)nc(Nc2cc3c(c(C4=CCNCCC4)c2)OCCC3)n1]

In some embodiments, the compound is selected from:

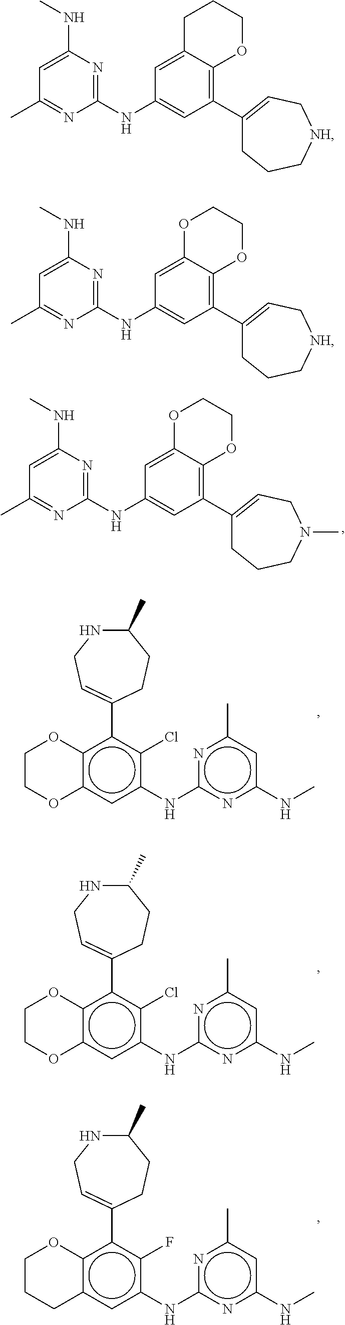

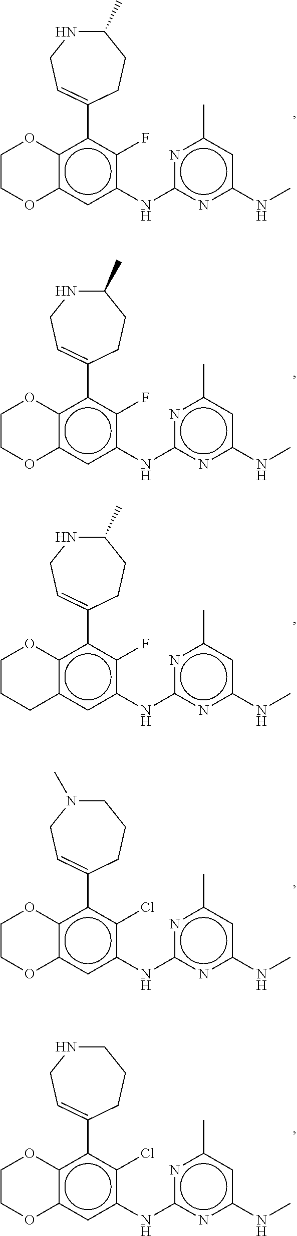

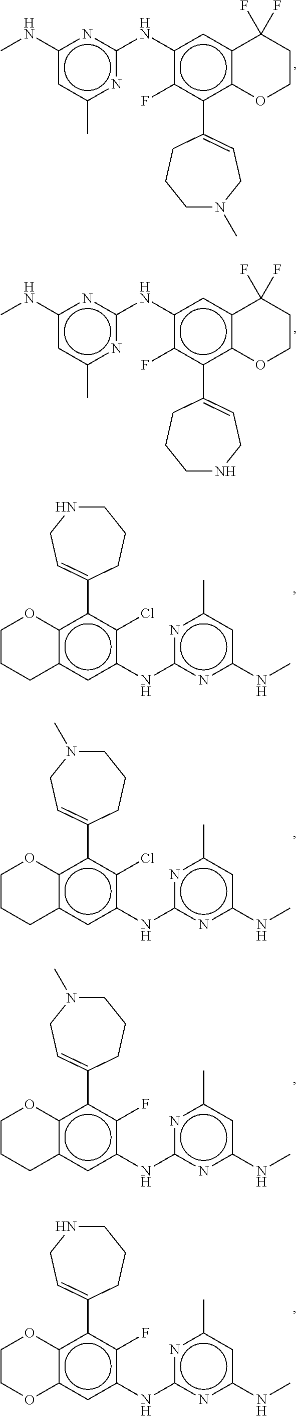

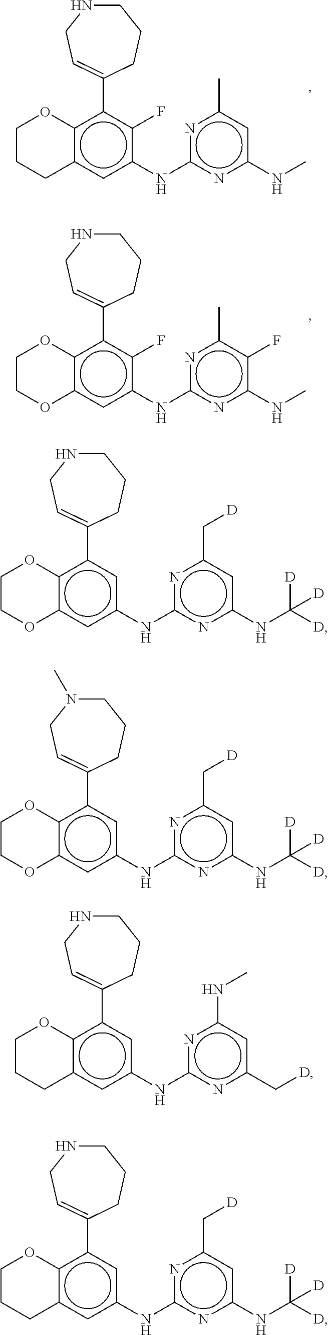

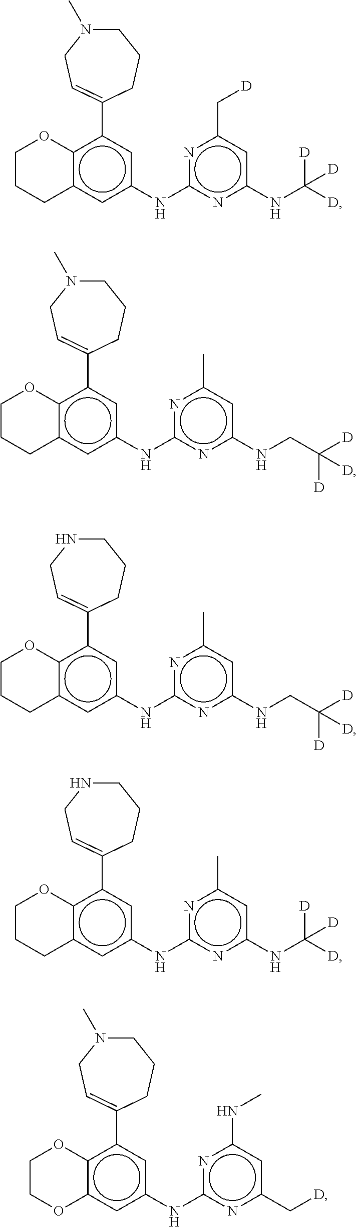

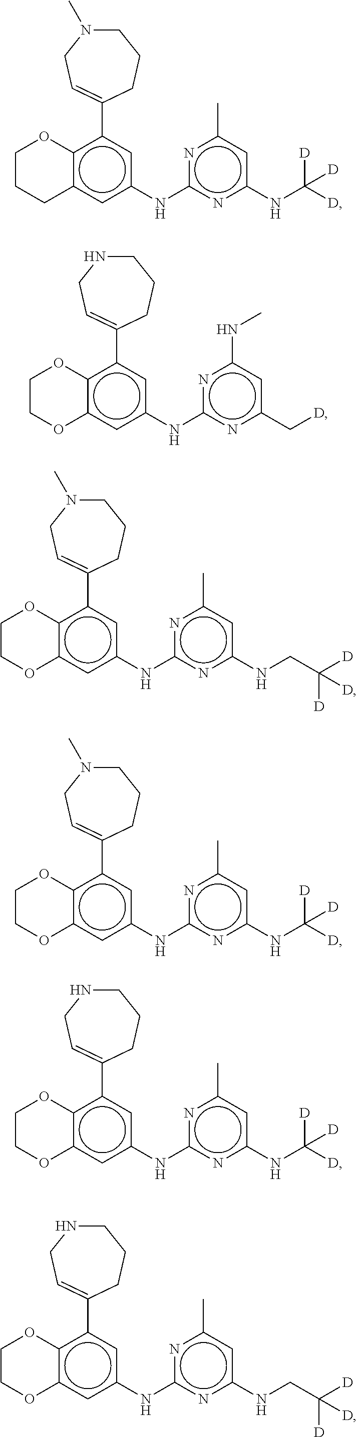

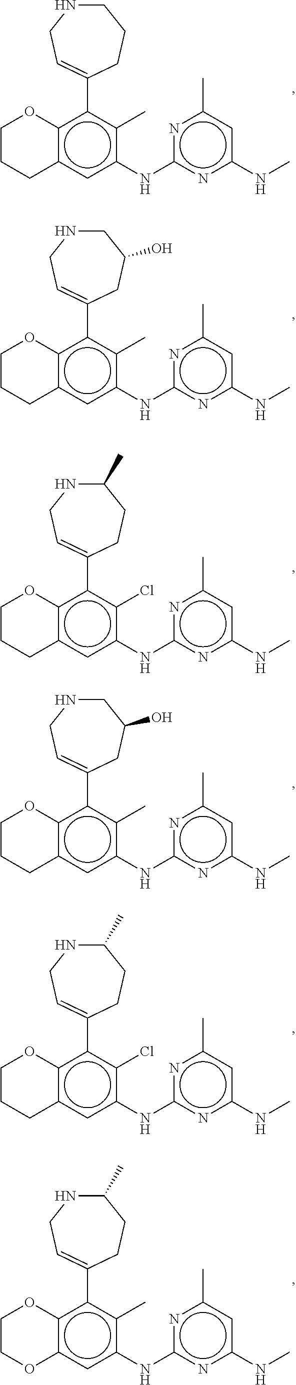

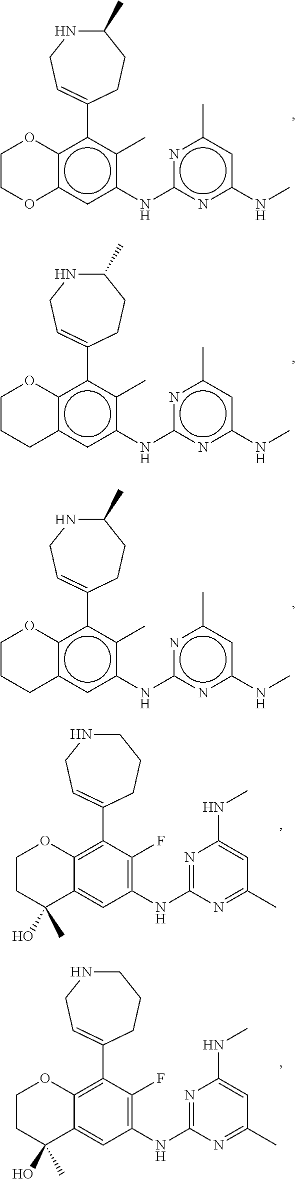

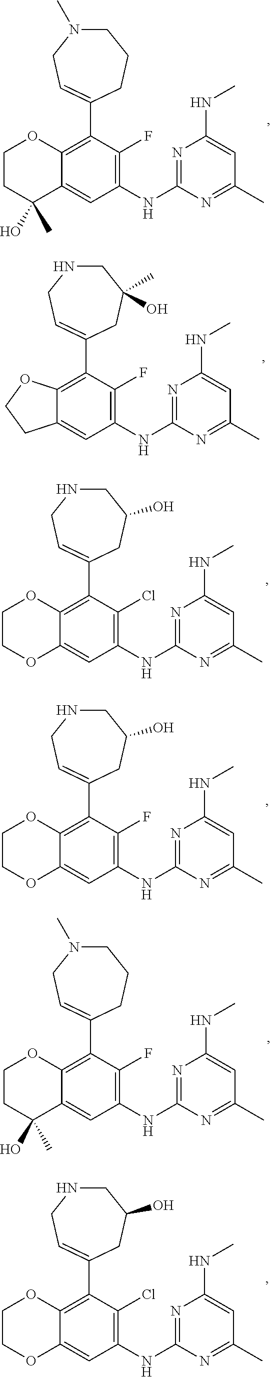

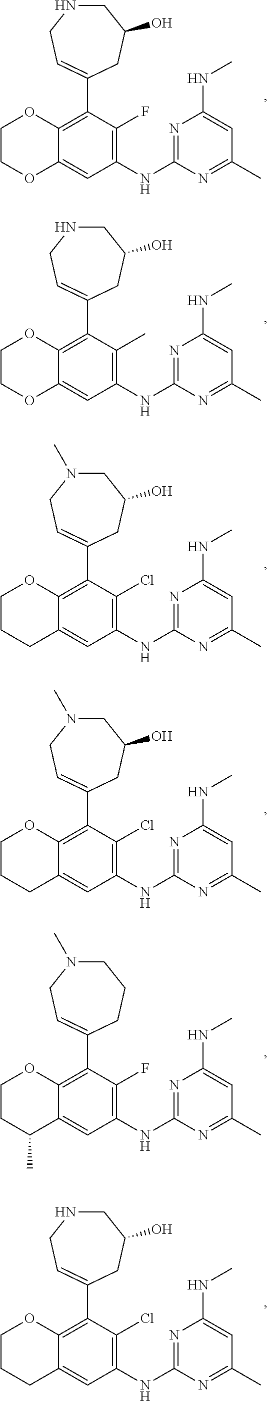 [SMILES: CNc1cc(C)nc(Nc2cc3c(c(C4=CCNC[C@@H](O)C4)c2F)OCCO3)n1 ; CNc1cc(C)nc(Nc2cc3c(c(C4=CCNC[C@H](O)C4)c2C)OCCO3)n1 ; CNc1cc(C)nc(Nc2cc3c(c(C4=CCN(C)C[C@H](O)C4)c2Cl)OCCC3)n1 ; CNc1cc(C)nc(Nc2cc3c(c(C4=CCN(C)C[C@@H](O)C4)c2Cl)OCCC3)n1 ; CNc1cc(C)nc(Nc2cc3c(c(C4=CCN(C)CCC4)c2F)OCC[C@H]3C)n1 ; CNc1cc(C)nc(Nc2cc3c(c(C4=CCNC[C@H](O)C4)c2Cl)OCCC3)n1]

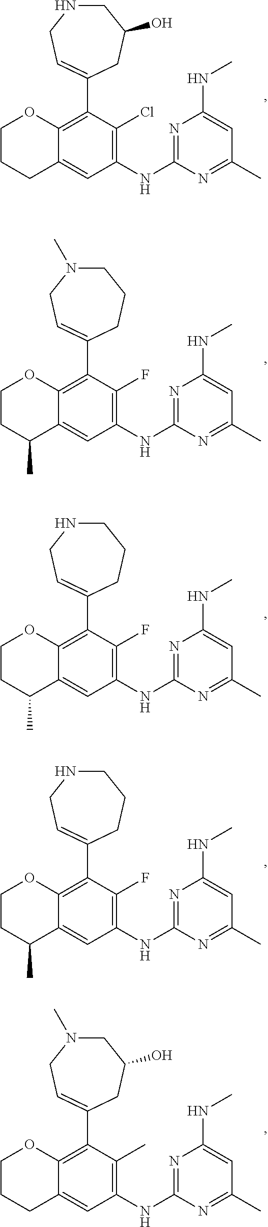

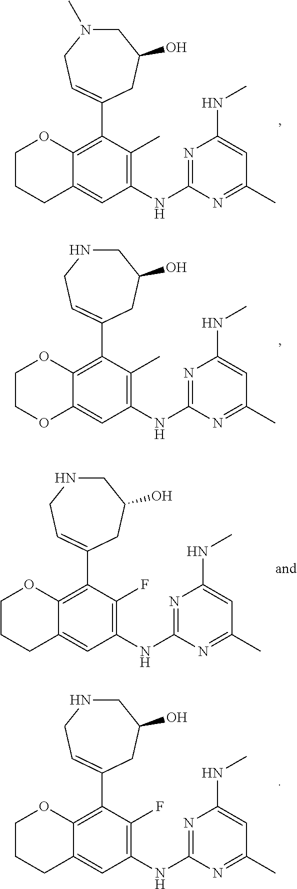 [SMILES: CNc1cc(C)nc(Nc2cc3c(c(C4=CCN(C)C[C@@H](O)C4)c2C)OCCC3)n1 ; CNc1cc(C)nc(Nc2cc3c(c(C4=CCNC[C@@H](O)C4)c2C)OCCO3)n1 ; CNc1cc(C)nc(Nc2cc3c(c(C4=CCNC[C@H](O)C4)c2F)OCCC3)n1 ; CNc1cc(C)nc(Nc2cc3c(c(C4=CCNC[C@@H](O)C4)c2F)OCCC3)n1]

In some embodiments, the compound is selected from:

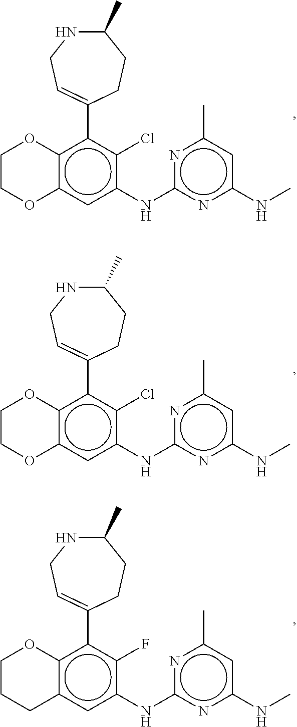

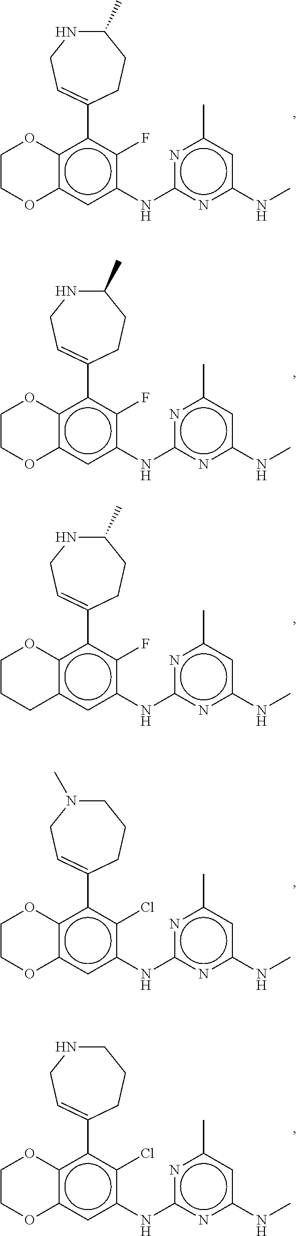

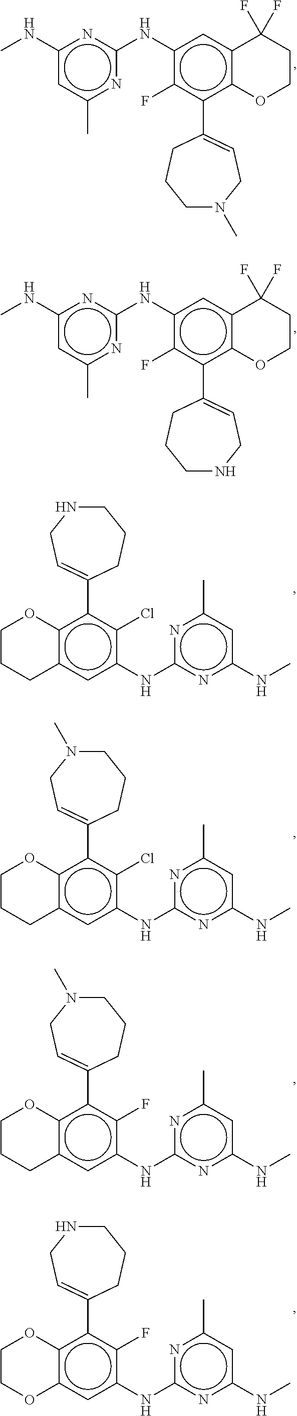

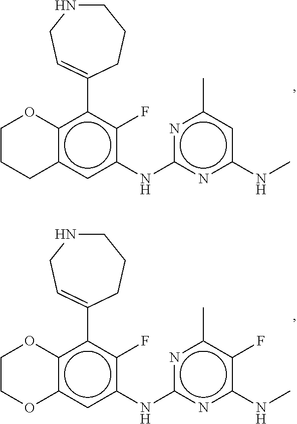

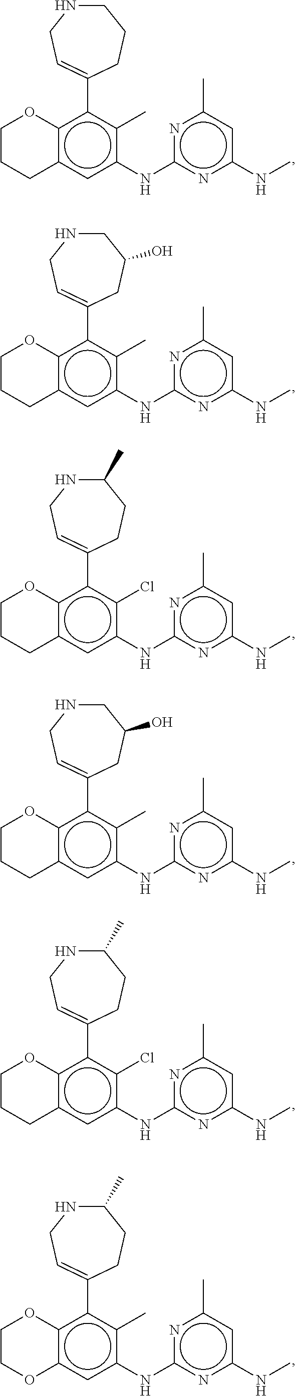

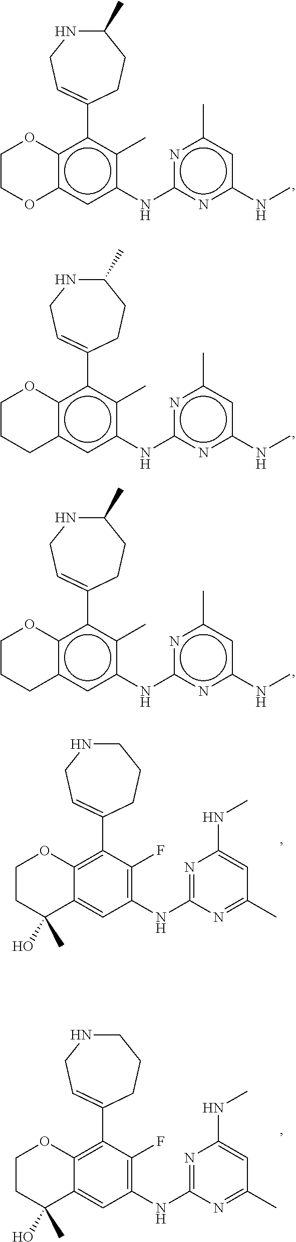

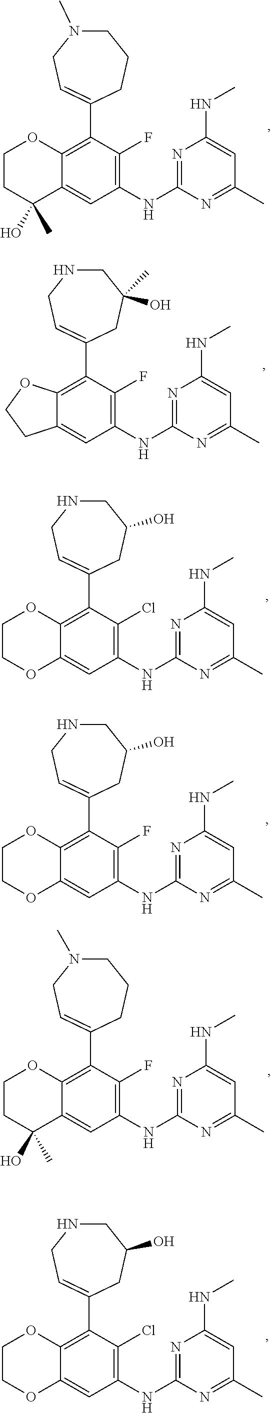

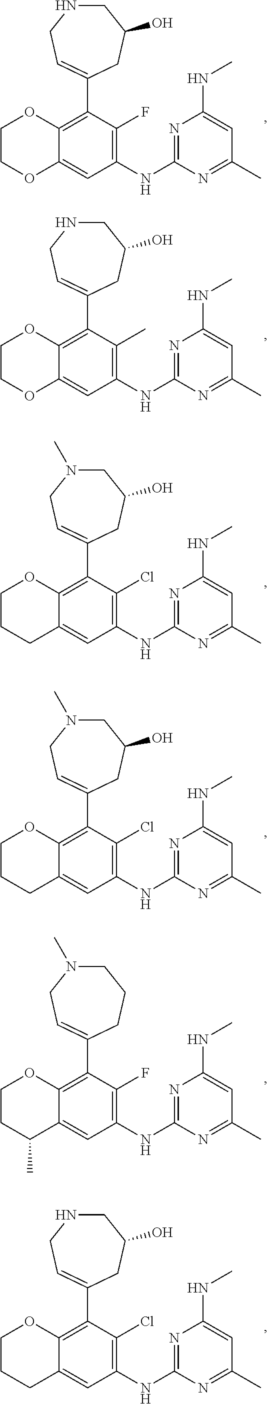 [SMILES: CNc1cc(C)nc(Nc2cc3c(c(C4=CCNC[C@@H](O)C4)c2F)OCCO3)n1 ; CNc1cc(C)nc(Nc2cc3c(c(C4=CCNC[C@H](O)C4)c2C)OCCO3)n1 ; CNc1cc(C)nc(Nc2cc3c(c(C4=CCN(C)C[C@H](O)C4)c2Cl)OCCC3)n1 ; CNc1cc(C)nc(Nc2cc3c(c(C4=CCN(C)C[C@@H](O)C4)c2Cl)OCCC3)n1 ; CNc1cc(C)nc(Nc2cc3c(c(C4=CCN(C)CCC4)c2F)OCC[C@H]3C)n1 ; CNc1cc(C)nc(Nc2cc3c(c(C4=CCNC[C@H](O)C4)c2Cl)OCCC3)n1]

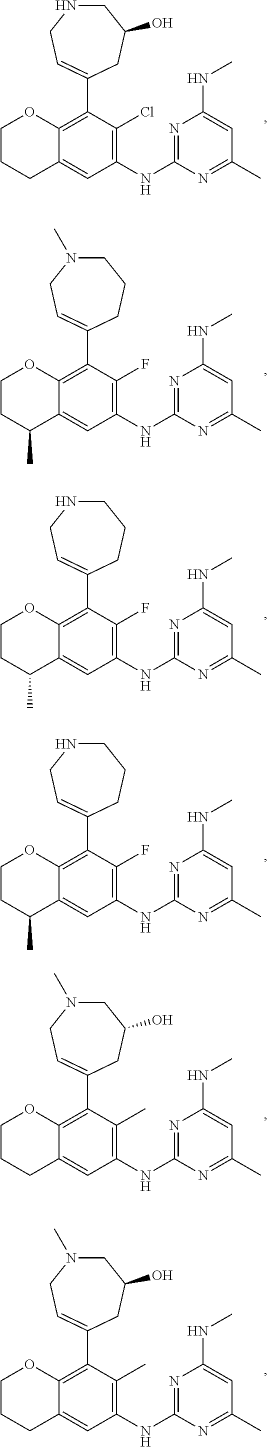

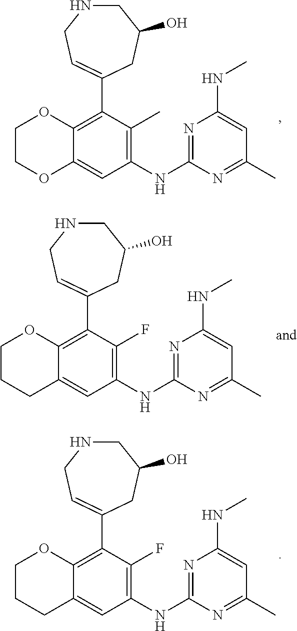

In some embodiments, the compound is selected from:

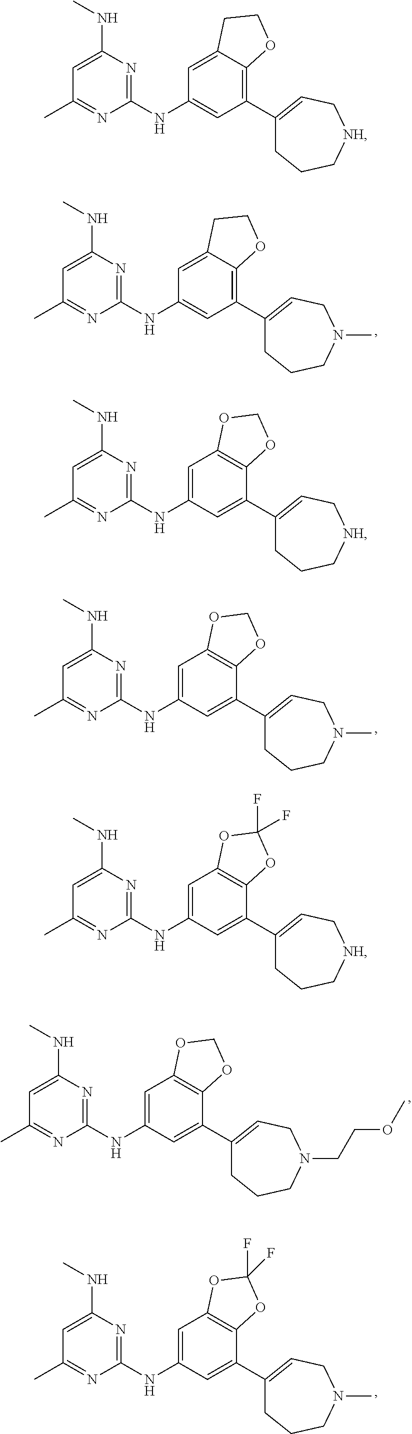

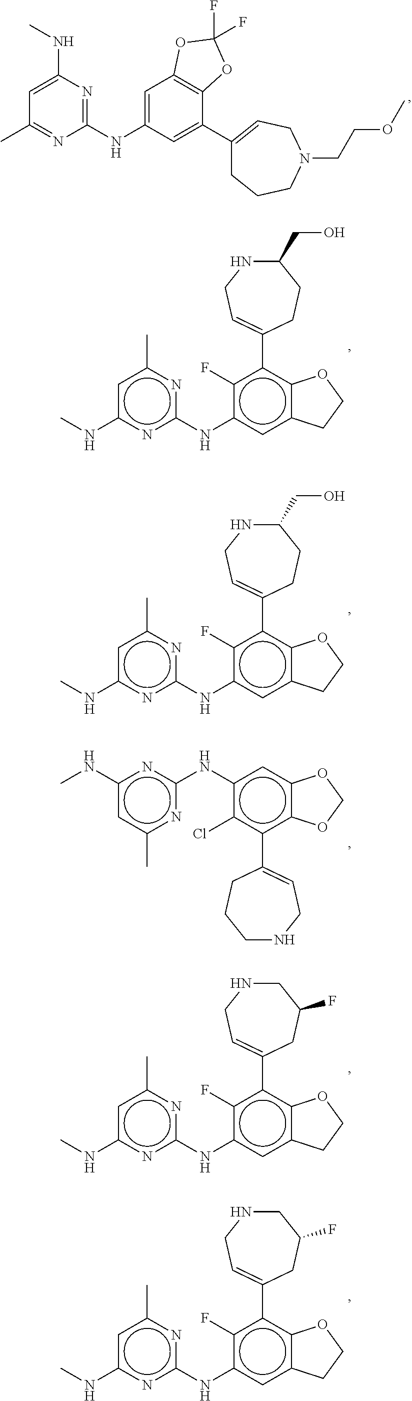

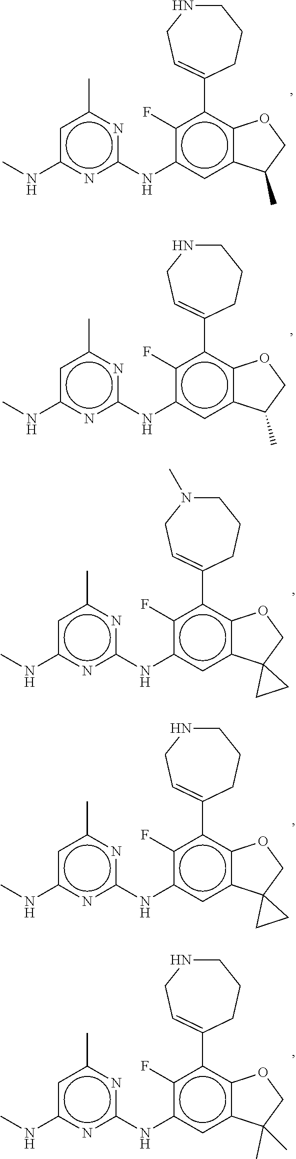

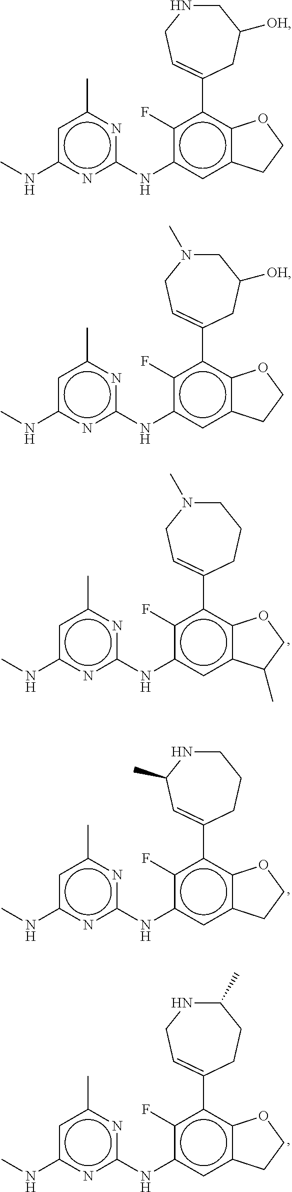

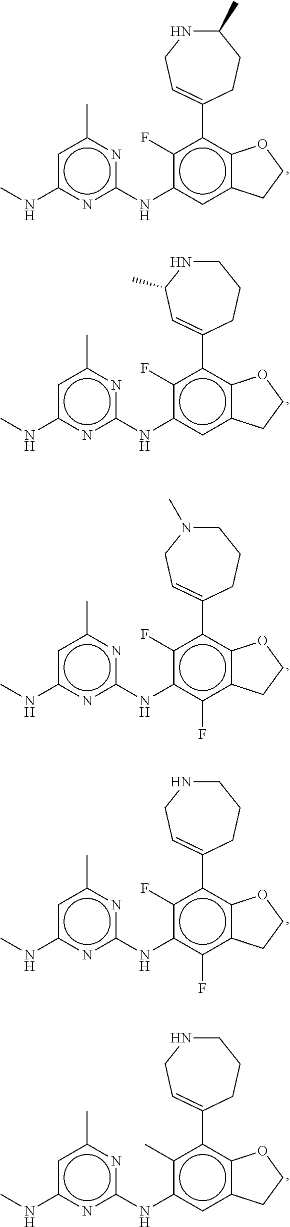

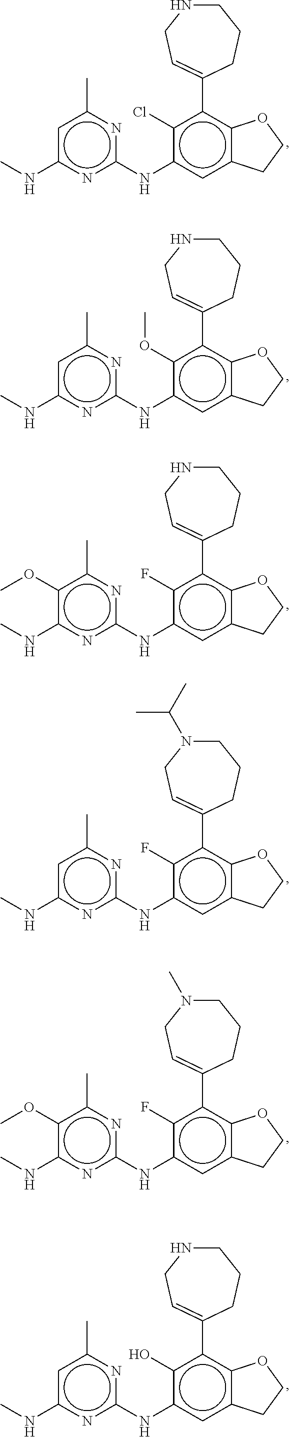

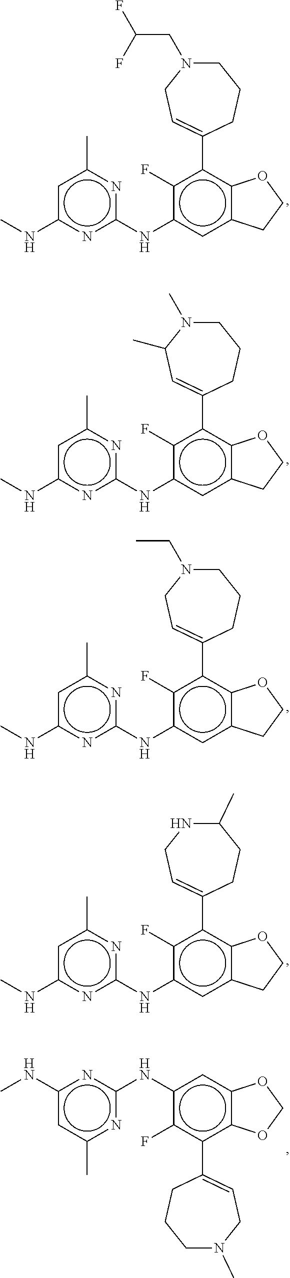

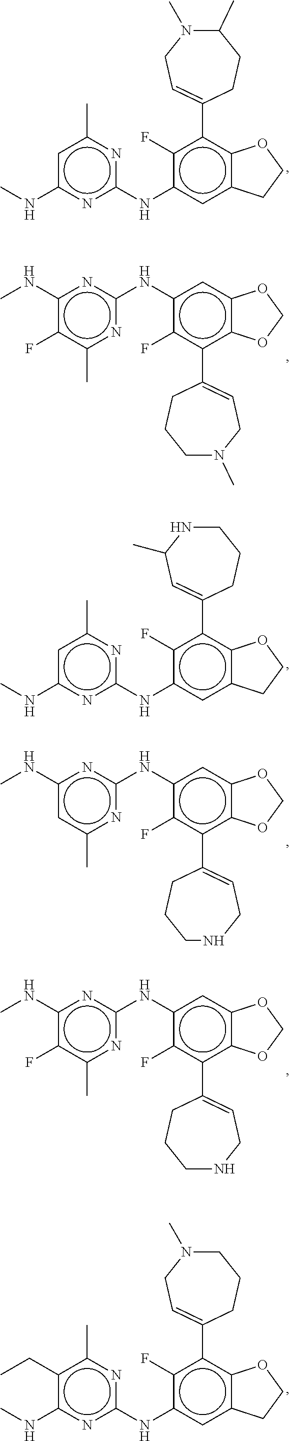

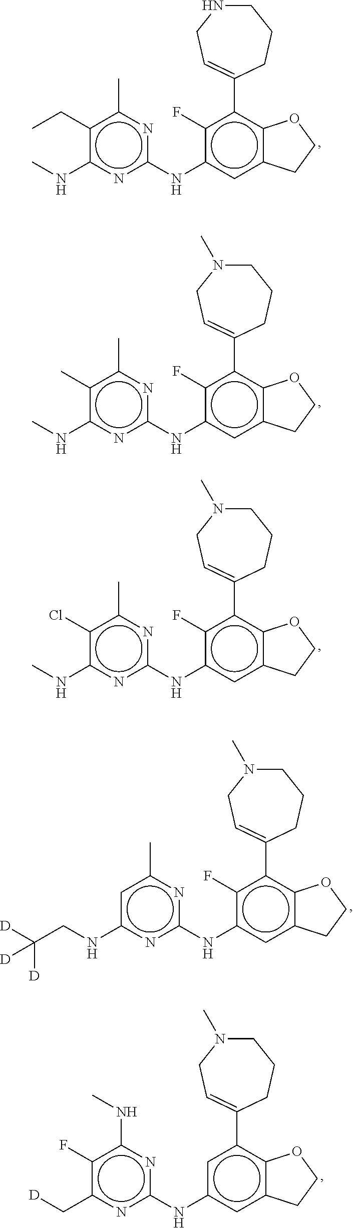

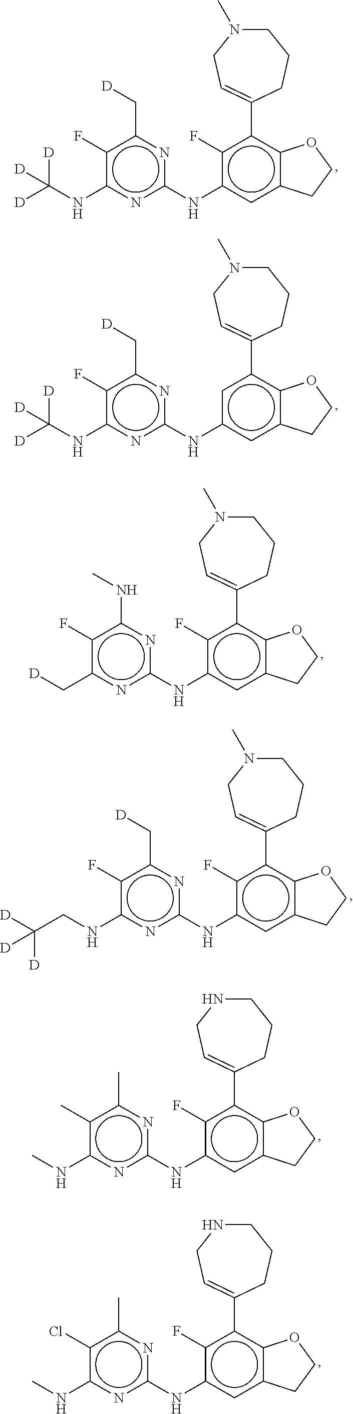

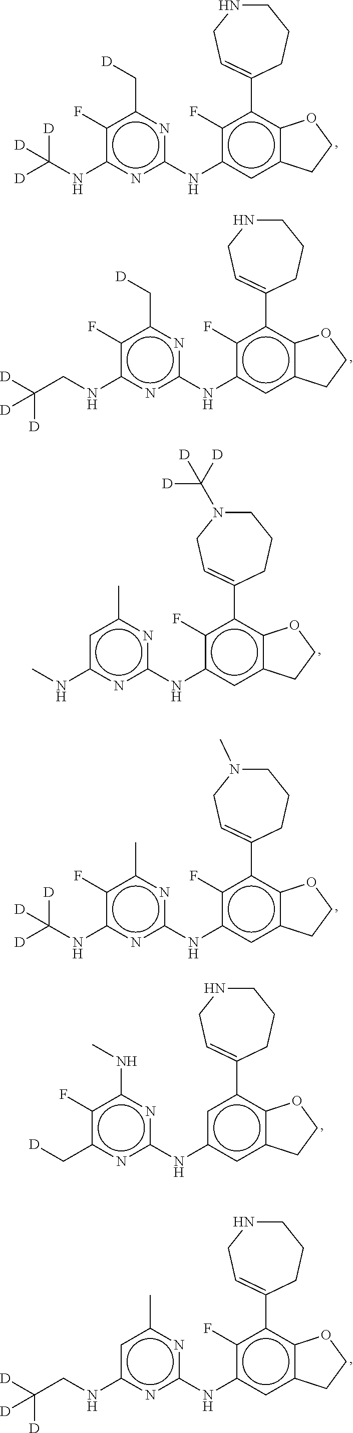

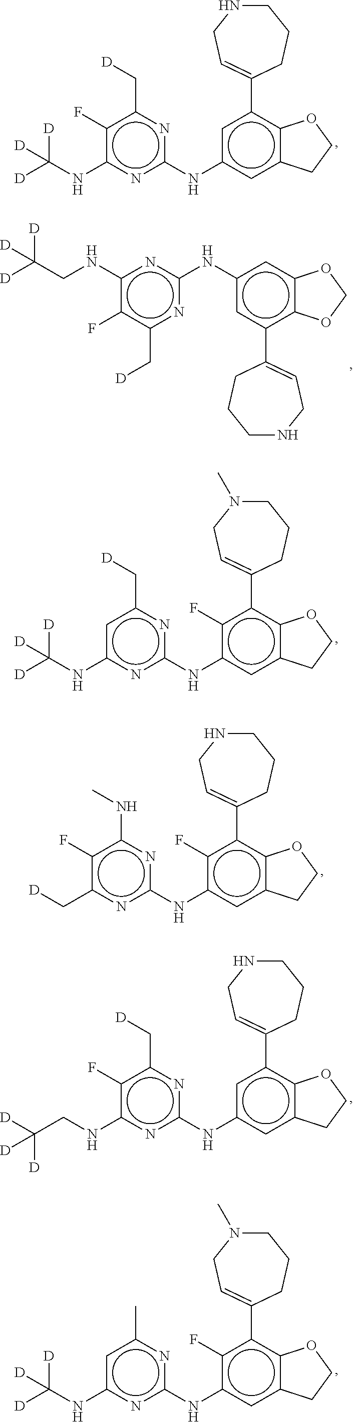

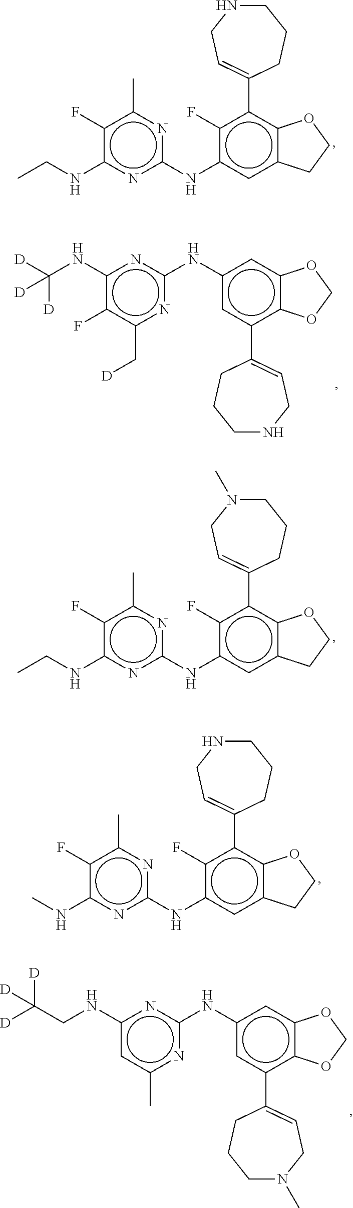

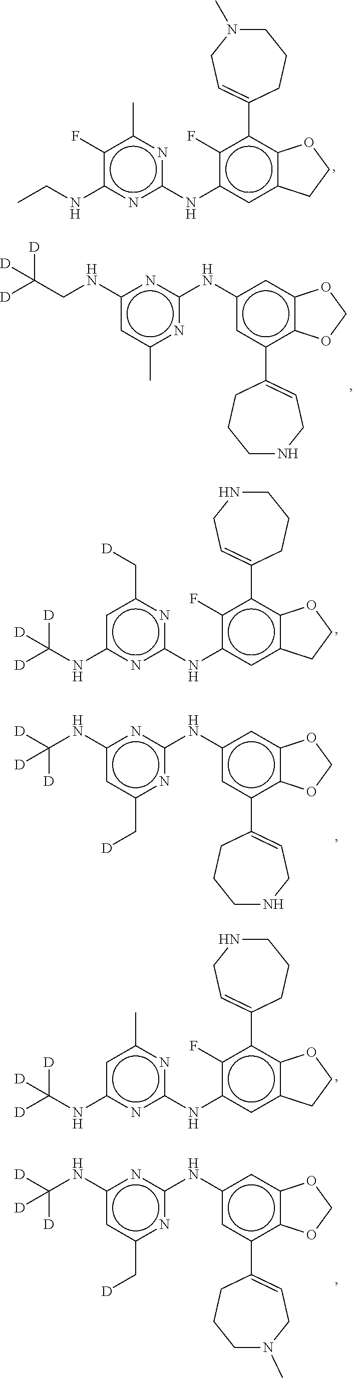

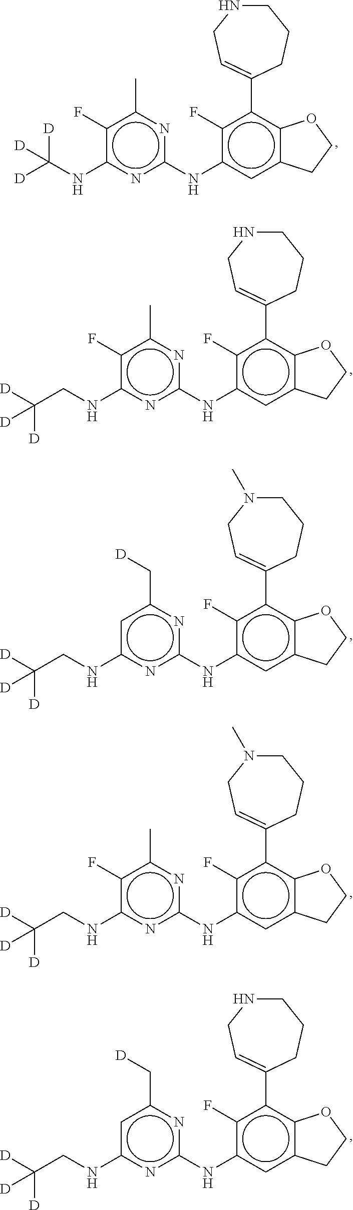

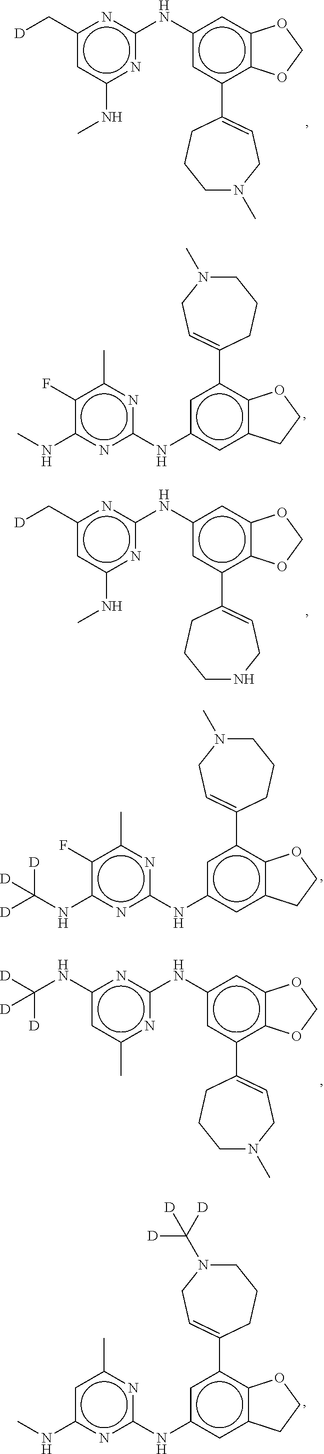

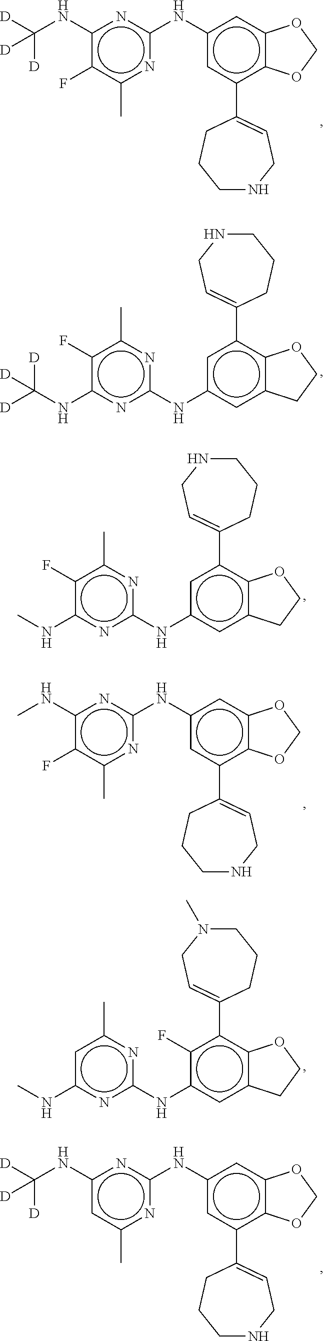

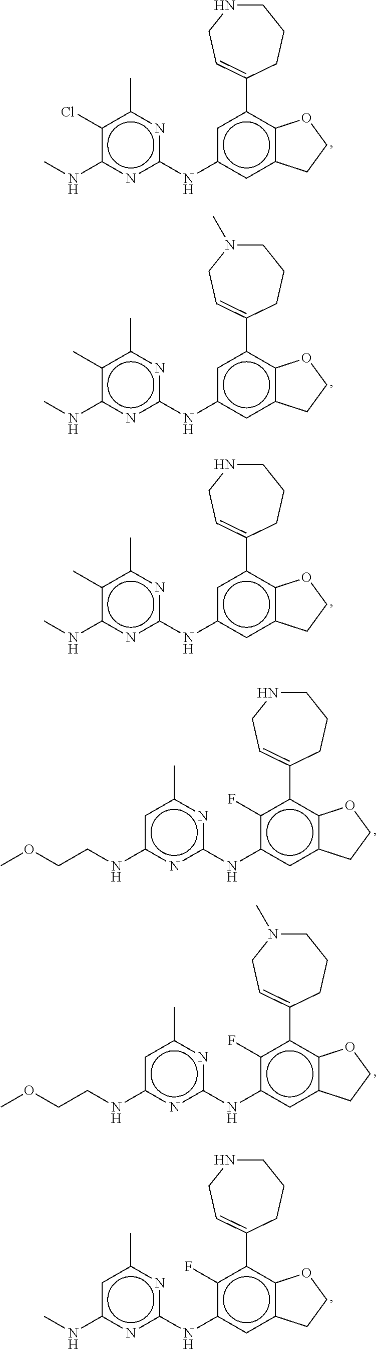

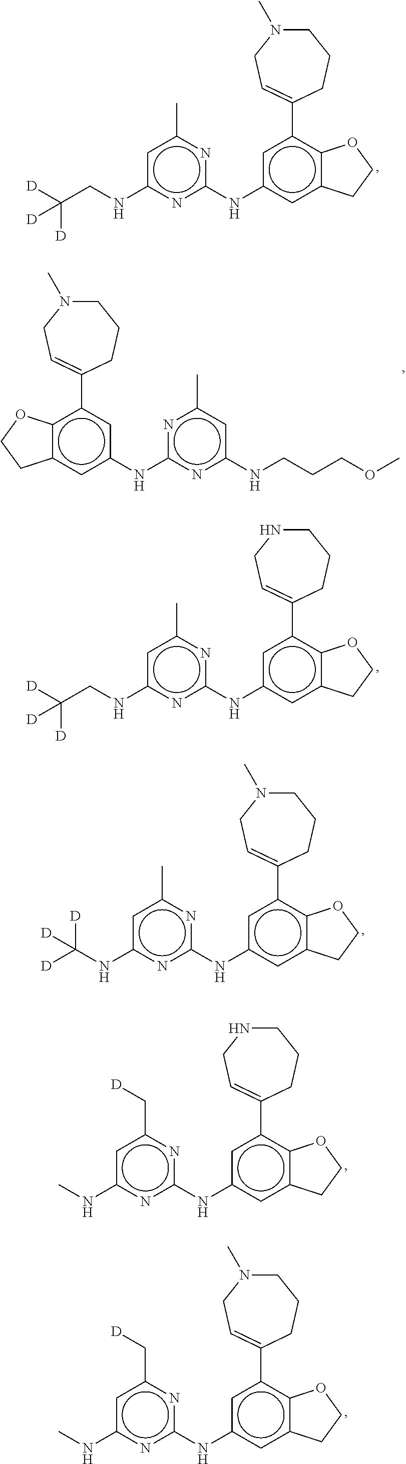

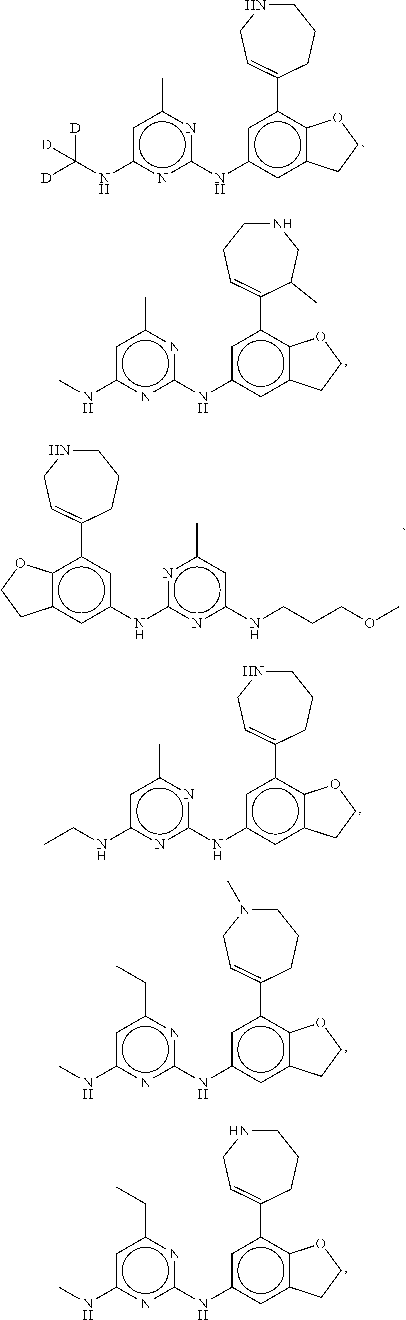

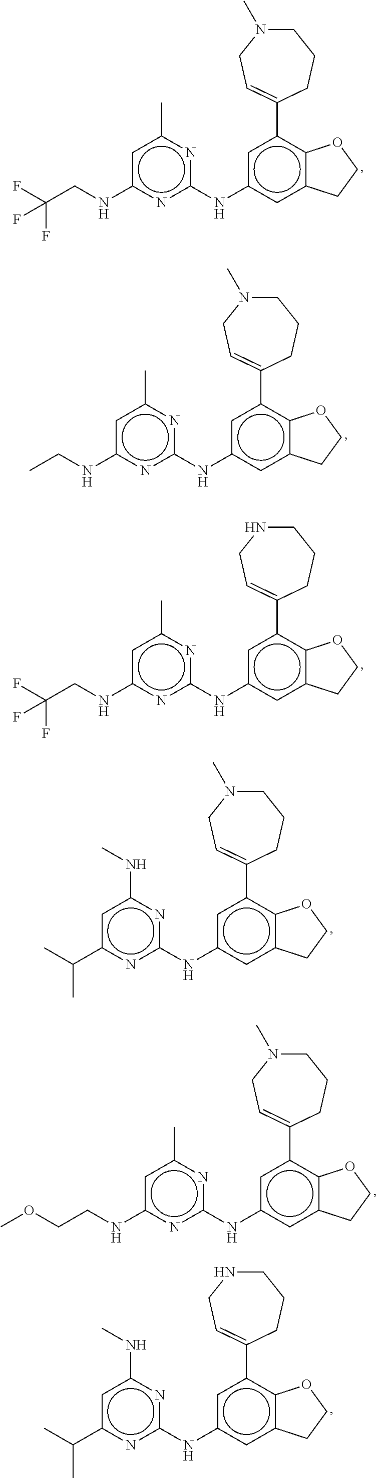

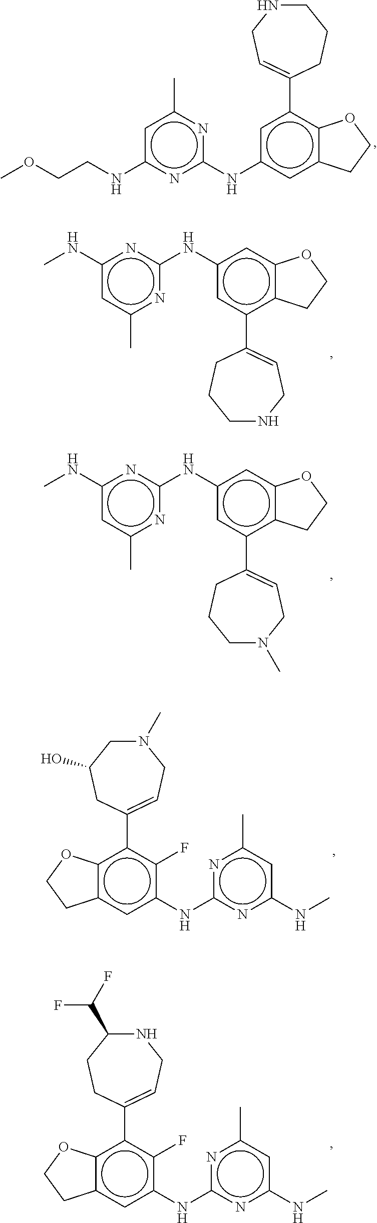

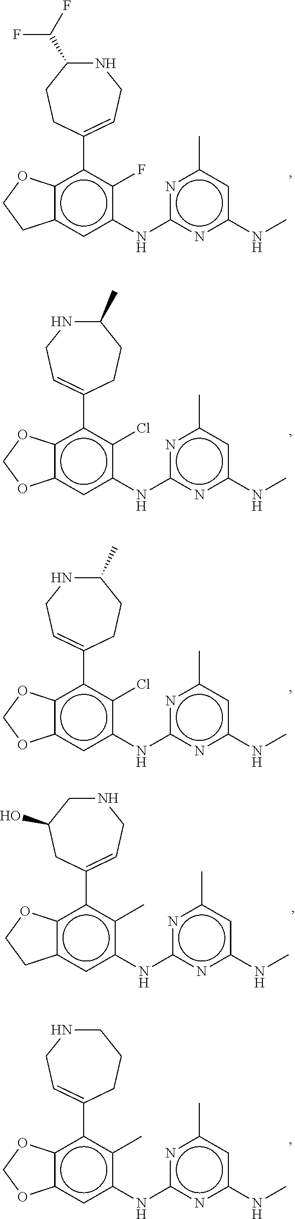

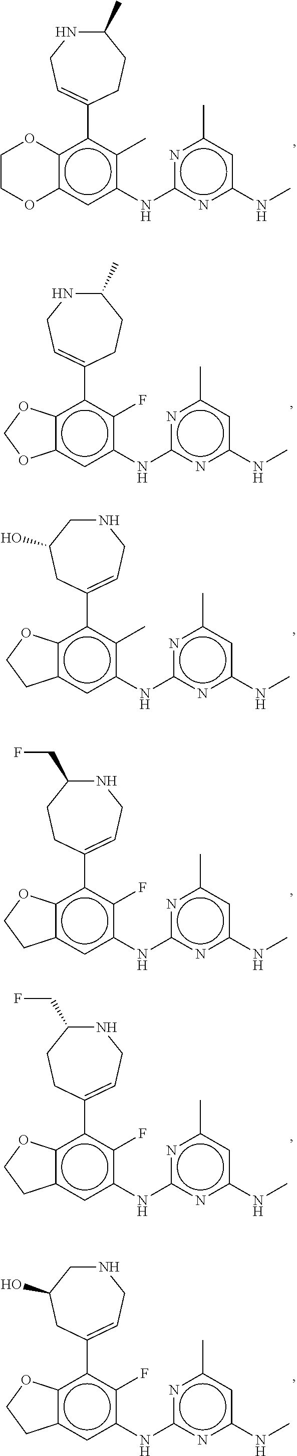

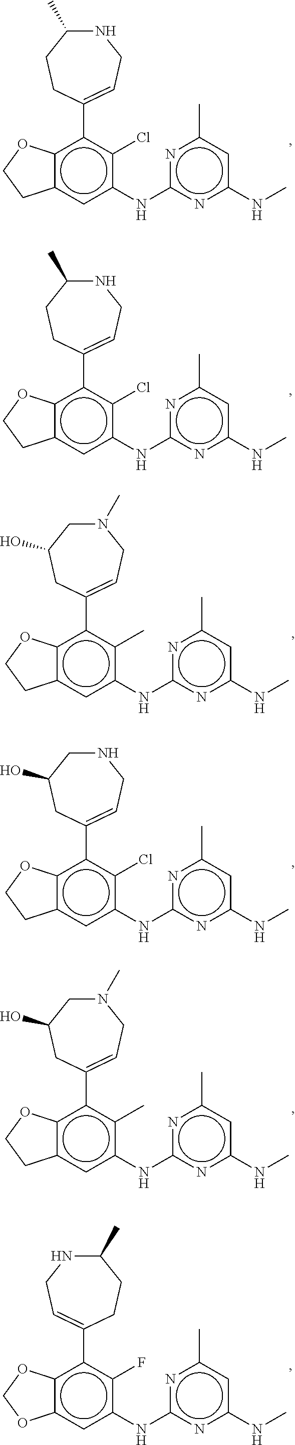

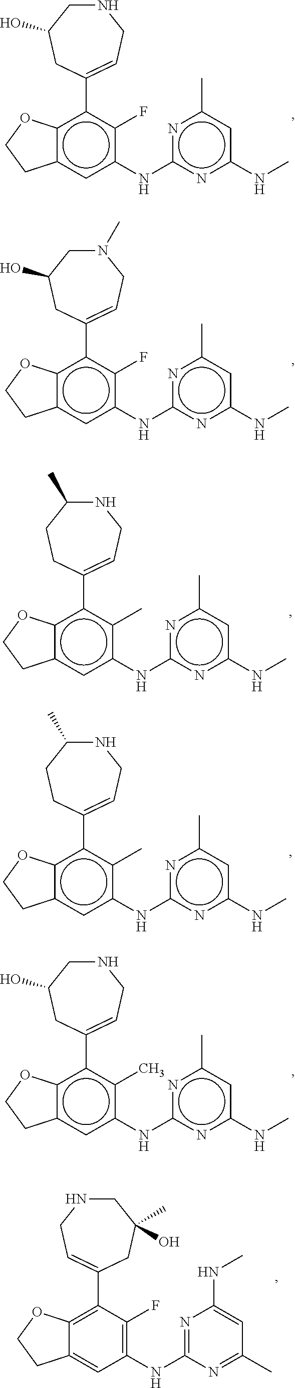

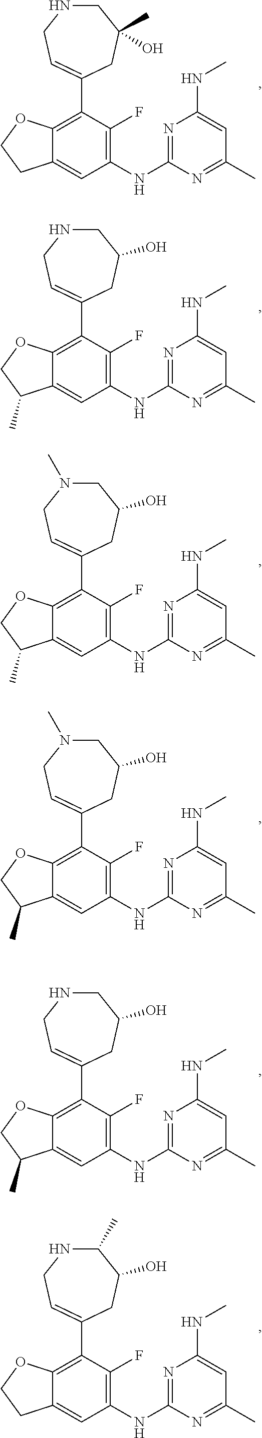

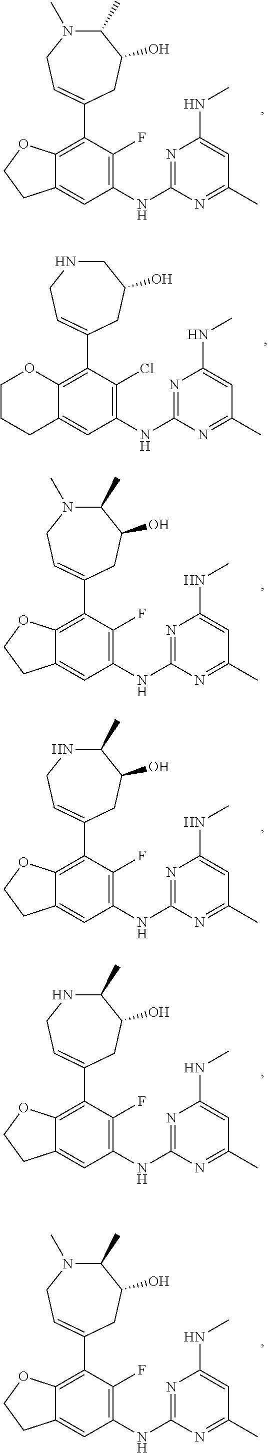 [SMILES: CNc1cc(C)nc(Nc2cc3c(c(C4=CCN(C)[C@H](C)[C@H](O)C4)c2F)OCC3)n1 ; CNc1cc(C)nc(Nc2cc3c(c(C4=CCNC[C@H](O)C4)c2Cl)OCCC3)n1 ; CNc1cc(C)nc(Nc2cc3c(c(C4=CCN(C)[C@@H](C)[C@@H](O)C4)c2F)OCC3)n1 ; CNc1cc(C)nc(Nc2cc3c(c(C4=CCN[C@@H](C)[C@@H](O)C4)c2F)OCC3)n1 ; CNc1cc(C)nc(Nc2cc3c(c(C4=CCN[C@@H](C)[C@H](O)C4)c2F)OCC3)n1 ; CNc1cc(C)nc(Nc2cc3c(c(C4=CCN(C)[C@@H](C)[C@H](O)C4)c2F)OCC3)n1]

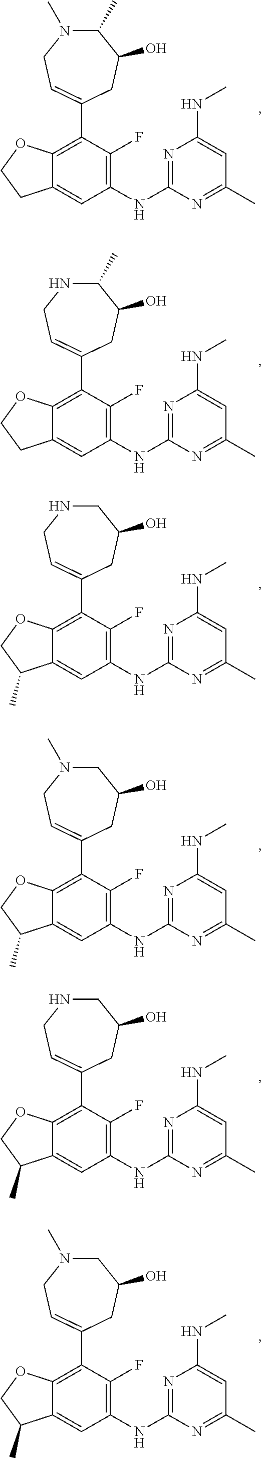 [SMILES: CNc1cc(C)nc(Nc2cc3c(c(C4=CCN(C)[C@H](C)[C@@H](O)C4)c2F)OCC3)n1 ; CNc1cc(C)nc(Nc2cc3c(c(C4=CCN[C@H](C)[C@@H](O)C4)c2F)OCC3)n1 ; CNc1cc(C)nc(Nc2cc3c(c(C4=CCNC[C@@H](O)C4)c2F)OC[C@H]3C)n1 ; CNc1cc(C)nc(Nc2cc3c(c(C4=CCN(C)C[C@@H](O)C4)c2F)OC[C@H]3C)n1 ; CNc1cc(C)nc(Nc2cc3c(c(C4=CCNC[C@@H](O)C4)c2F)OC[C@@H]3C)n1 ; CNc1cc(C)nc(Nc2cc3c(c(C4=CCN(C)C[C@@H](O)C4)c2F)OC[C@@H]3C)n1]

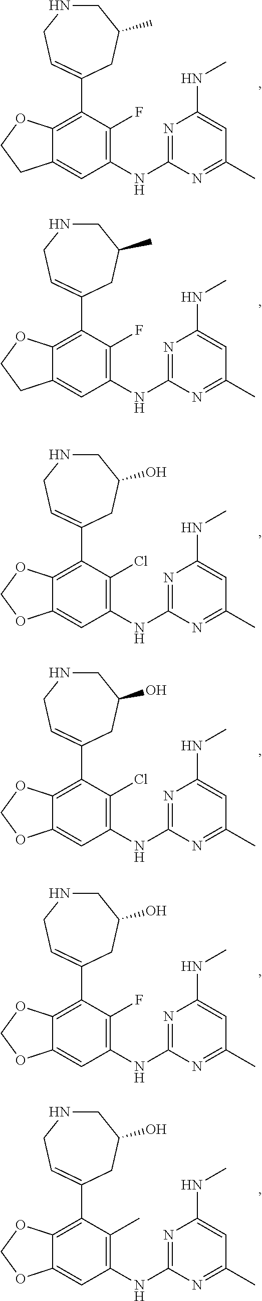 [SMILES: CNc1cc(C)nc(Nc2cc3c(c(C4=CCNC[C@H](C)C4)c2F)OCC3)n1 ; CNc1cc(C)nc(Nc2cc3c(c(C4=CCNC[C@@H](C)C4)c2F)OCC3)n1 ; CNc1cc(C)nc(Nc2cc3c(c(C4=CCNC[C@H](O)C4)c2Cl)OCO3)n1 ; CNc1cc(C)nc(Nc2cc3c(c(C4=CCNC[C@@H](O)C4)c2Cl)OCO3)n1 ; CNc1cc(C)nc(Nc2cc3c(c(C4=CCNC[C@H](O)C4)c2F)OCO3)n1 ; CNc1cc(C)nc(Nc2cc3c(c(C4=CCNC[C@H](O)C4)c2C)OCO3)n1]

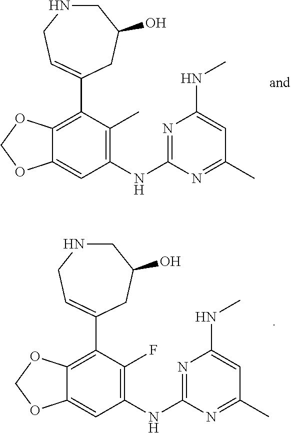 [SMILES: CNc1cc(C)nc(Nc2cc3c(c(C4=CCNC[C@@H](O)C4)c2C)OCO3)n1 ; CNc1cc(C)nc(Nc2cc3c(c(C4=CCNC[C@@H](O)C4)c2F)OCO3)n1]

In some embodiments, the compound is selected from:

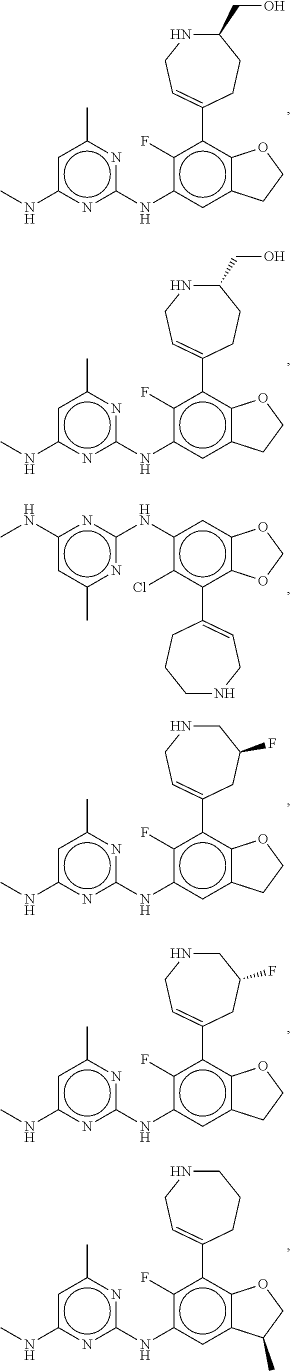

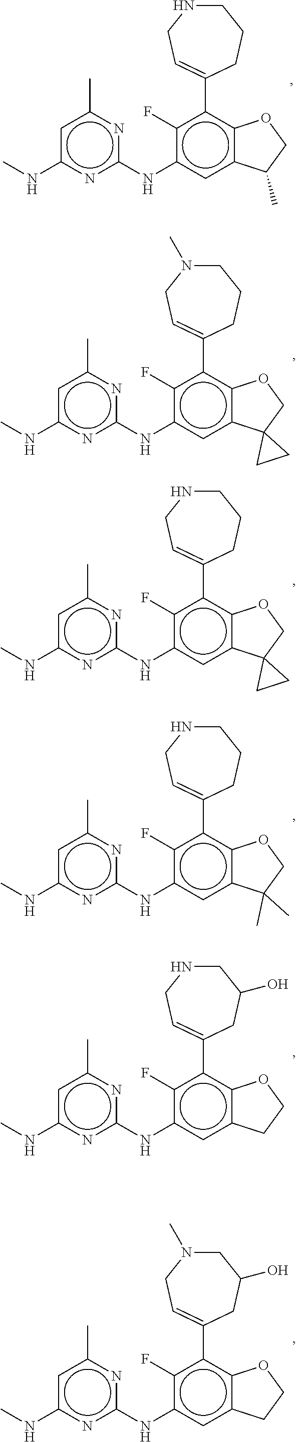

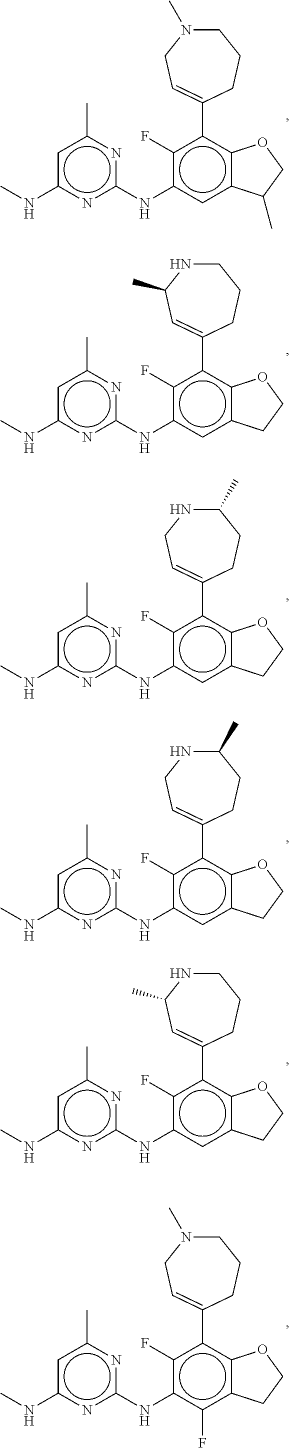

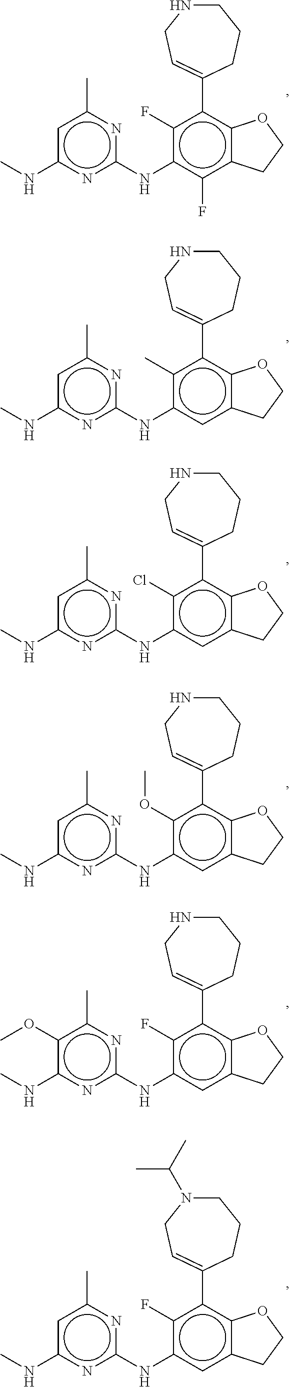

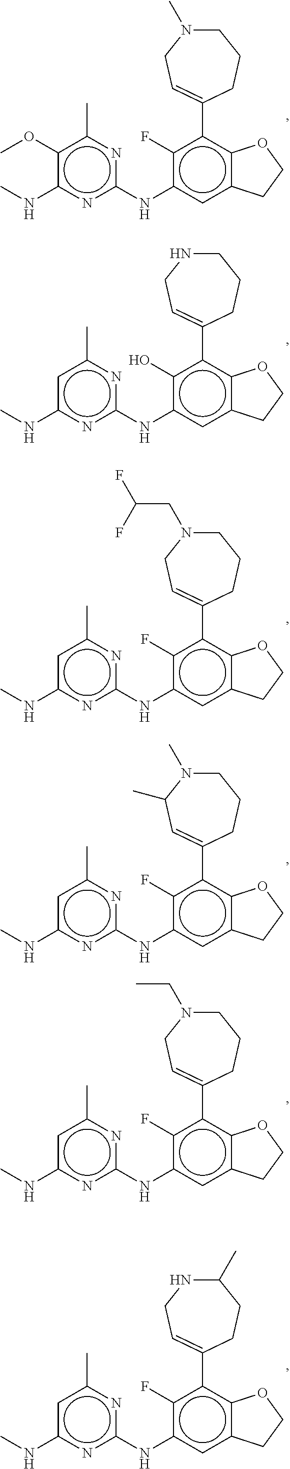

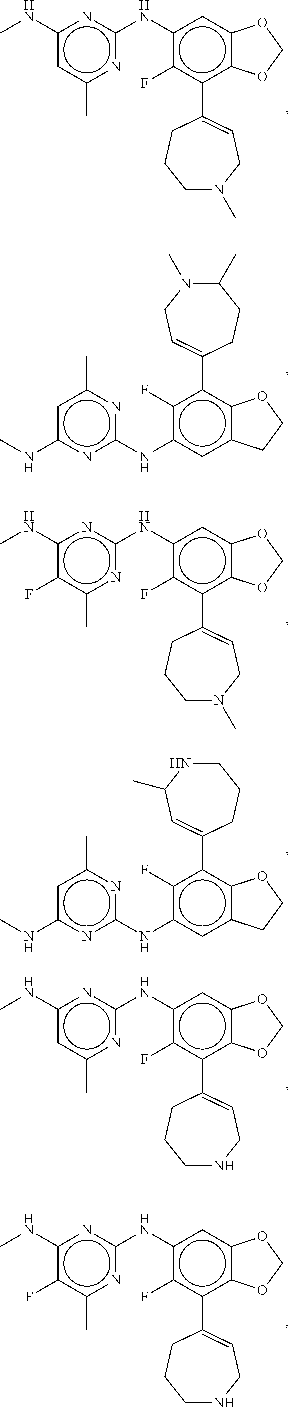

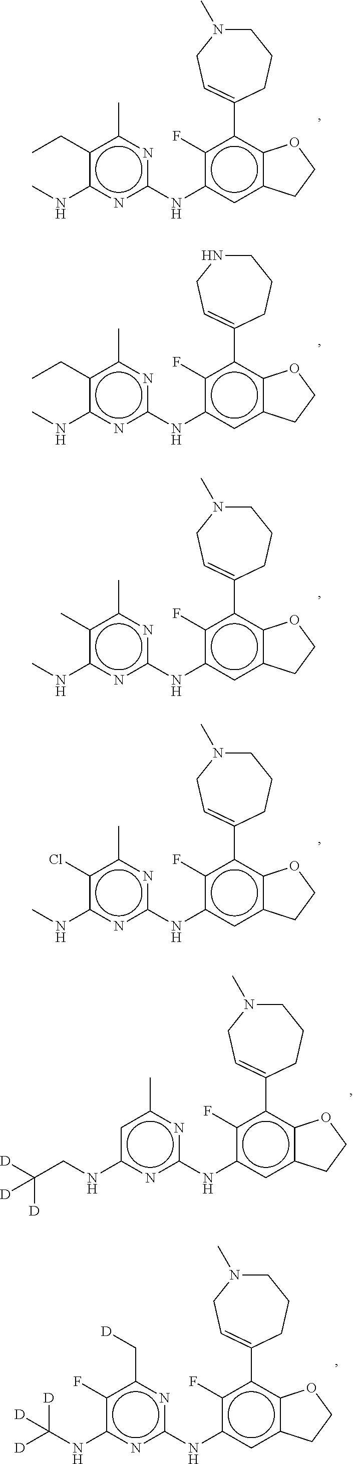

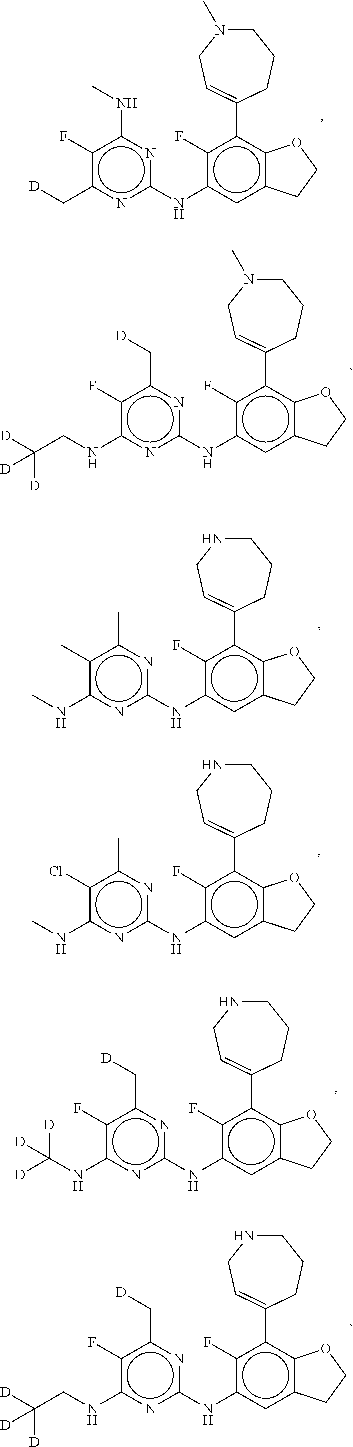

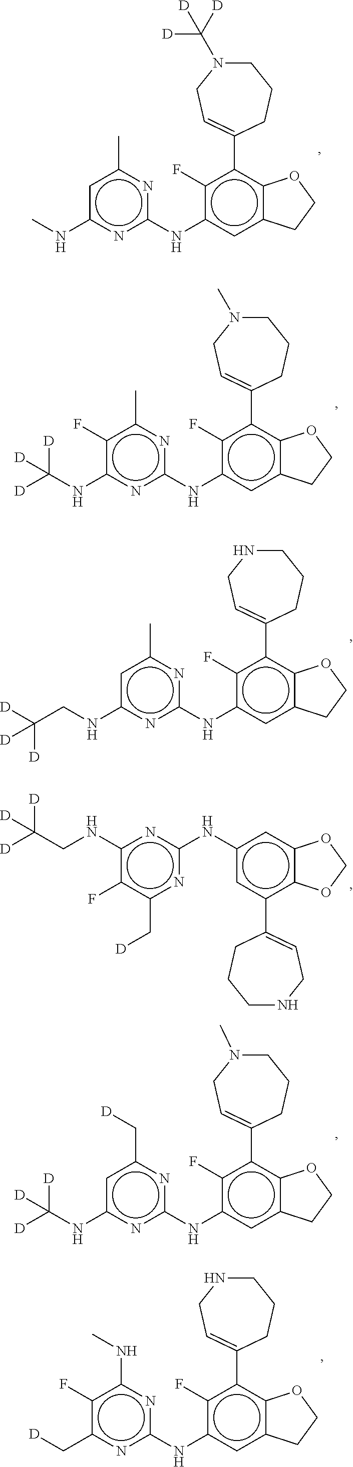

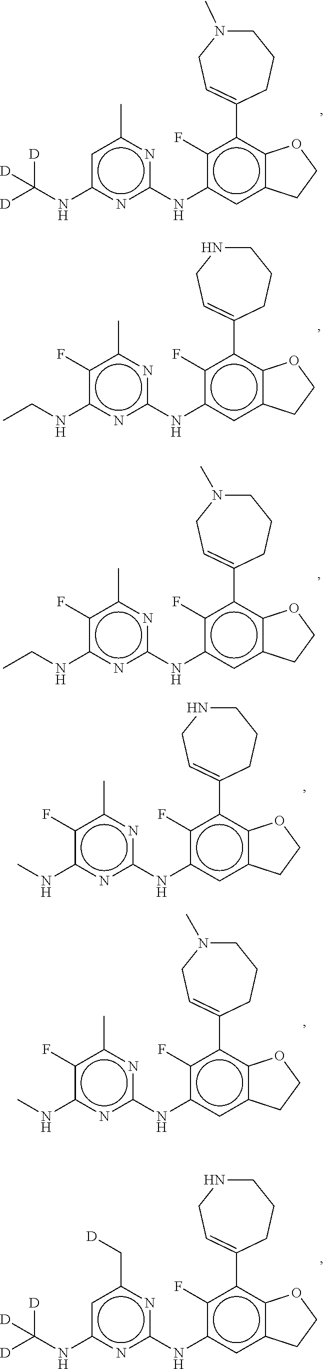

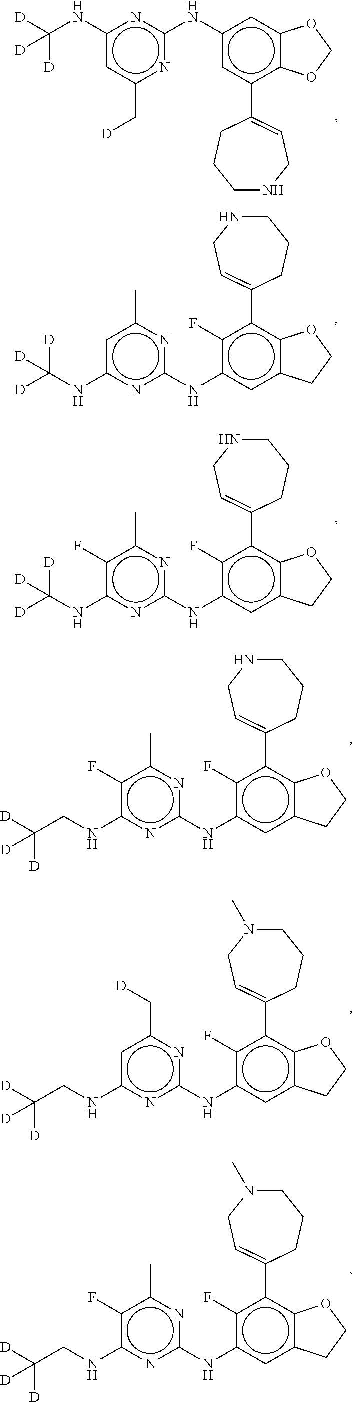

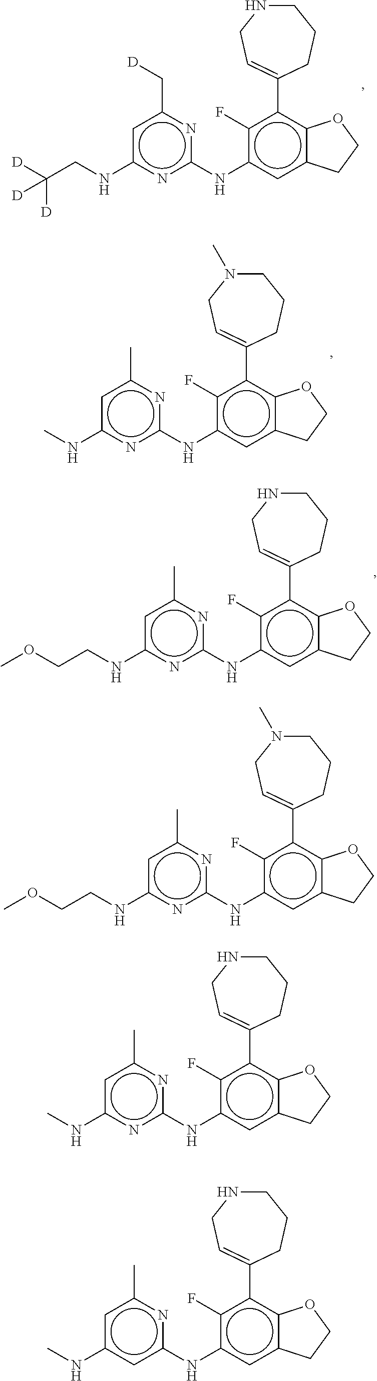

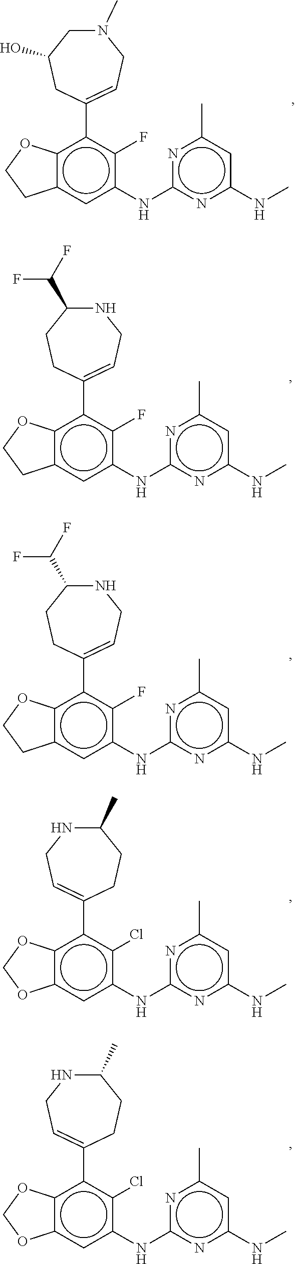

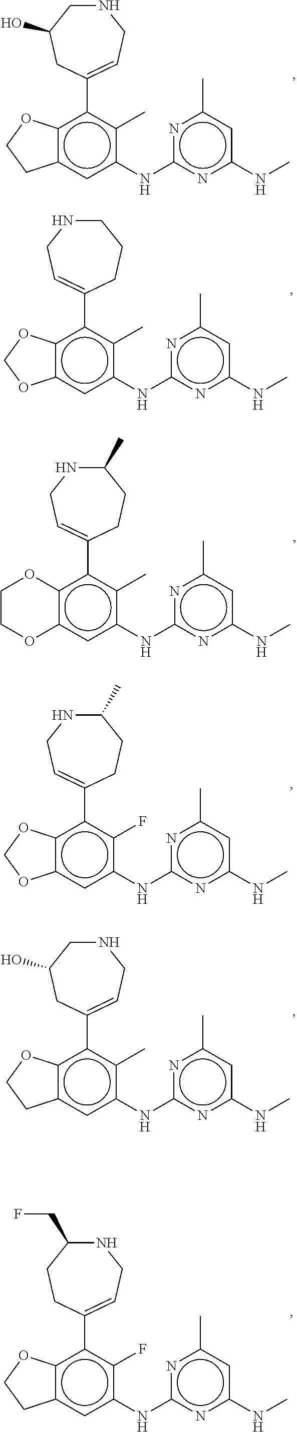

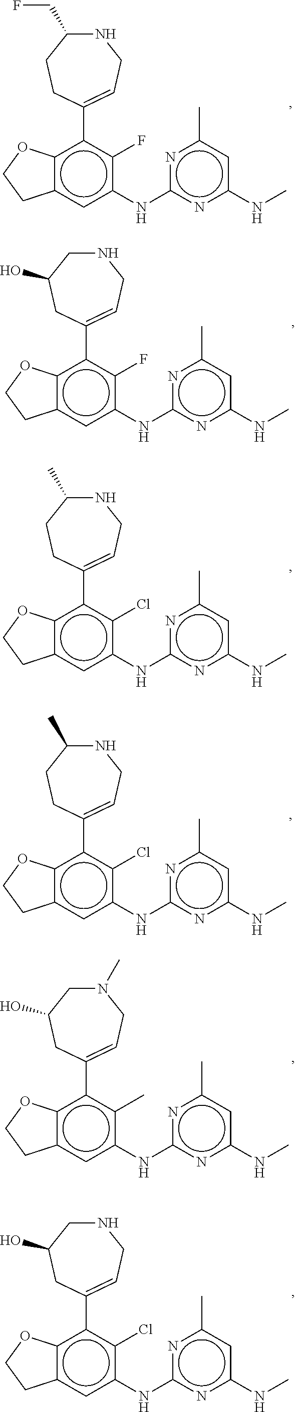

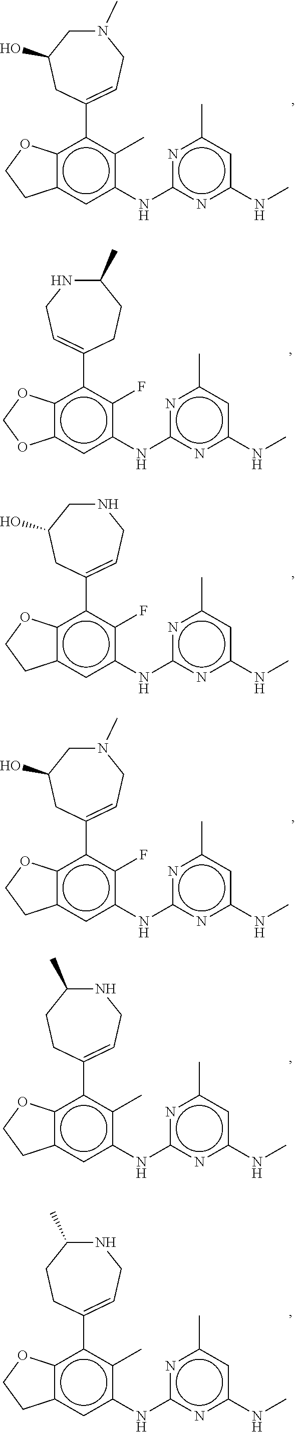

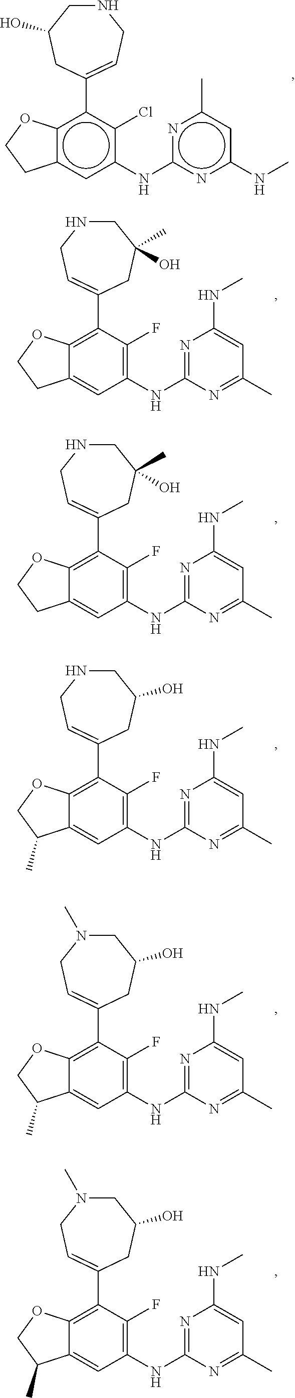

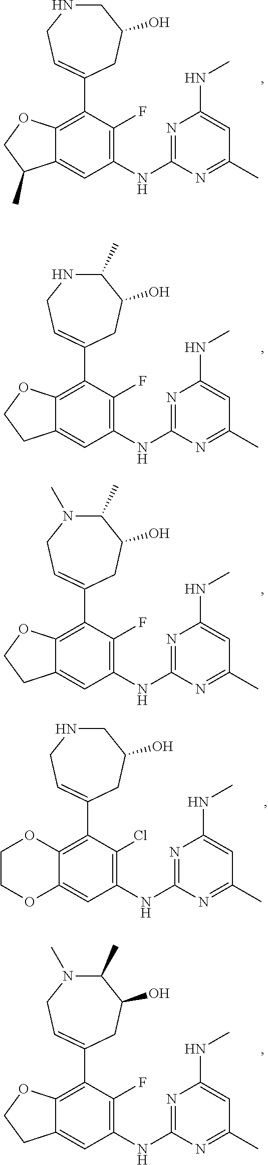

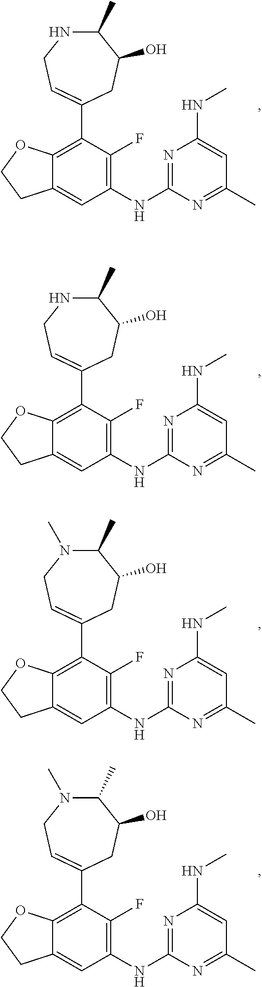 [SMILES: CNc1cc(C)nc(Nc2cc3c(c(C4=CCN[C@@H](C)[C@@H](O)C4)c2F)OCC3)n1 ; CNc1cc(C)nc(Nc2cc3c(c(C4=CCN[C@@H](C)[C@H](O)C4)c2F)OCC3)n1 ; CNc1cc(C)nc(Nc2cc3c(c(C4=CCN(C)[C@@H](C)[C@H](O)C4)c2F)OCC3)n1 ; CNc1cc(C)nc(Nc2cc3c(c(C4=CCN(C)[C@H](C)[C@@H](O)C4)c2F)OCC3)n1]

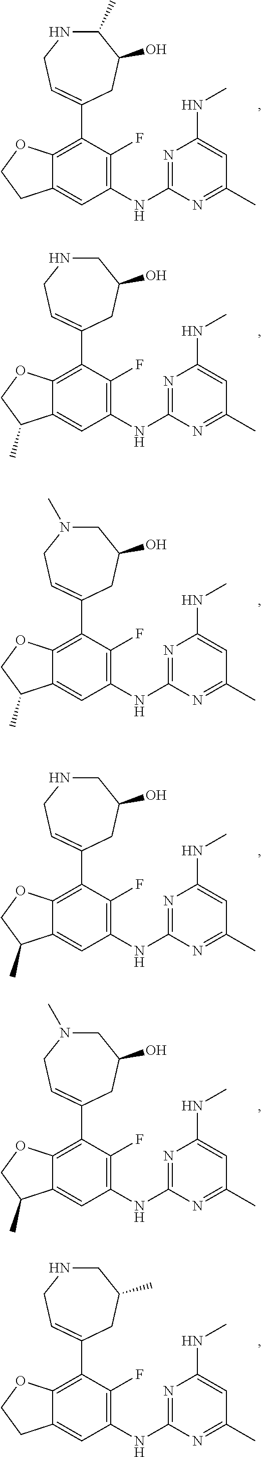

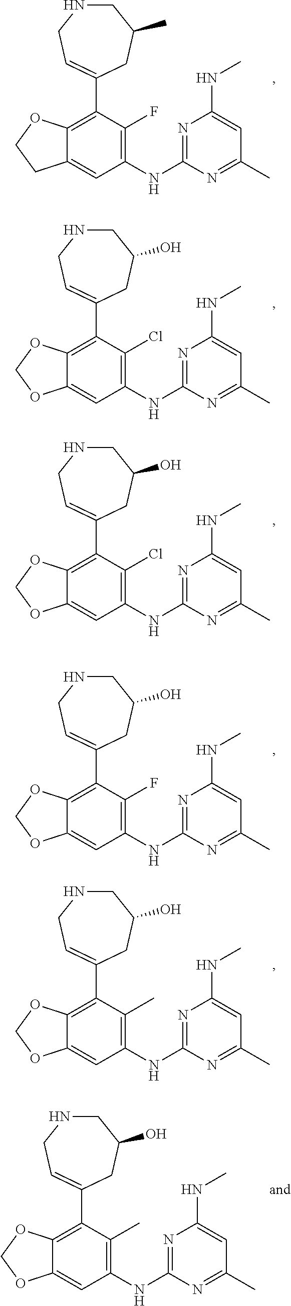

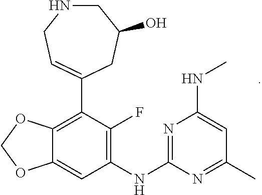 [SMILES: CNc1cc(C)nc(Nc2cc3c(c(C4=CCNC[C@@H](O)C4)c2F)OCO3)n1]

In some embodiments, provided herein is a composition comprising a compound described herein and a pharmaceutically acceptable carrier.

In some embodiments, the compound is a compound identified in Table 1 below or a pharmaceutically acceptable salt thereof.

Unless otherwise indicated, the absolute stereochemistry of all chiral atoms is as depicted. Compounds marked with (or) are single enantiomers wherein the absolute stereochemistry was arbitrarily assigned (e.g., based on chiral SFC elution as described in the Examples section). Compounds marked with (and) are mixtures of enantiomers wherein the relative stereochemistry is as shown. Compounds that have a stereogenic center where the configuration is not indicated in the structure as depicted and that are not marked in the “stereochemistry” column are mixtures of enantiomers. Compounds marked with (abs) are single enantiomers wherein the absolute stereochemistry is as indicated.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

TABLE 1
Exemplary compounds

| Compound | Stereo- | |
|---|---|---|
| No. | chemistry | Structure |
| 102 | | 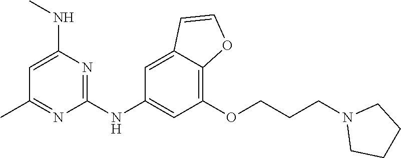 [SMILES: CNc1cc(C)nc(Nc2cc(OCCCN3CCCC3)c3occc3c2)n1] |
| 103 | | 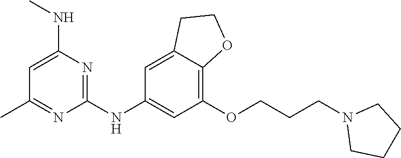 [SMILES: CNc1cc(C)nc(Nc2cc3c(c(OCCCN4CCCC4)c2)OCC3)n1] |
| 104 | | 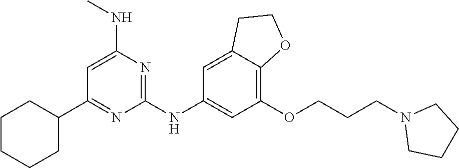 [SMILES: CNc1cc(C2CCCCC2)nc(Nc2cc3c(c(OCCCN4CCCC4)c2)OCC3)n1] |
| 105 | | 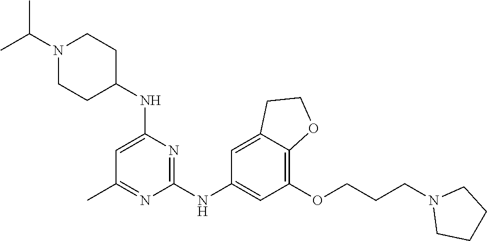 [SMILES: Cc1cc(NC2CCN(C(C)C)CC2)nc(Nc2cc3c(c(OCCCN4CCCC4)c2)OCC3)n1] |
| 106 | | 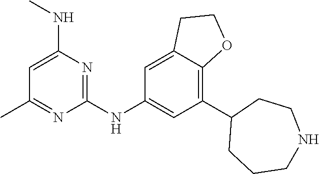 [SMILES: CNc1cc(C)nc(Nc2cc3c(c(C4CCCNCC4)c2)OCC3)n1] |
| 107 | | 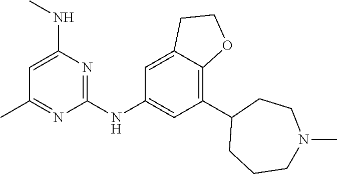 |
| 108 | | 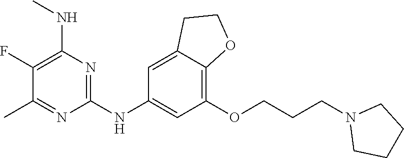 [SMILES: CNc1nc(Nc2cc3c(c(OCCCN4CCCC4)c2)OCC3)nc(C)c1F] |
| 109 | | 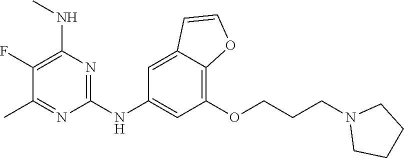 [SMILES: CNc1nc(Nc2cc(OCCCN3CCCC3)c3occc3c2)nc(C)c1F] |
| 110 | | 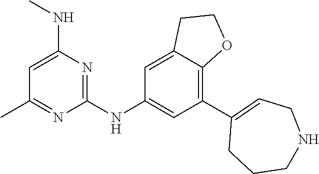 [SMILES: CNc1cc(C)nc(Nc2cc3c(c(C4=CCNCCC4)c2)OCC3)n1] |
| 111 | | 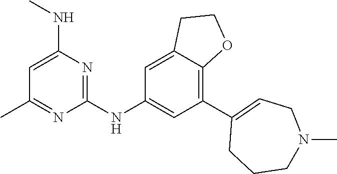 |
| 112 | | 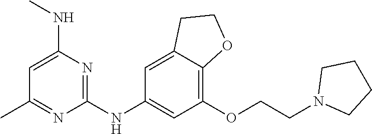 [SMILES: CNc1cc(C)nc(Nc2cc3c(c(OCCN4CCCC4)c2)OCC3)n1] |
| 113 | | 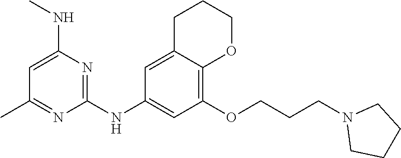 [SMILES: CNc1cc(C)nc(Nc2cc3c(c(OCCCN4CCCC4)c2)OCCC3)n1] |
| 114 | | 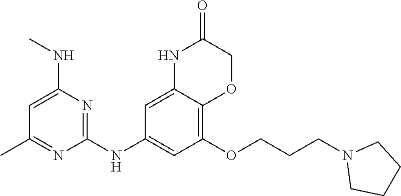 [SMILES: CNc1cc(C)nc(Nc2cc3c(c(OCCCN4CCCC4)c2)OCC(=O)N3)n1] |
| 115 | | 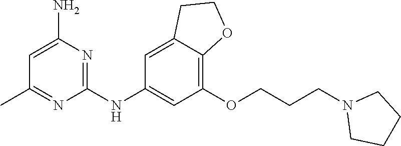 [SMILES: Cc1cc(N)nc(Nc2cc3c(c(OCCCN4CCCC4)c2)OCC3)n1] |
| 116 | | 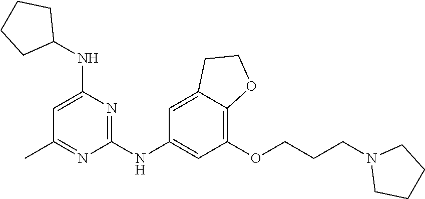 [SMILES: Cc1cc(NC2CCCC2)nc(Nc2cc3c(c(OCCCN4CCCC4)c2)OCC3)n1] |
| 117 | | 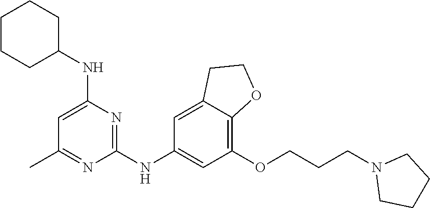 [SMILES: Cc1cc(NC2CCCCC2)nc(Nc2cc3c(c(OCCCN4CCCC4)c2)OCC3)n1] |
| 118 | | 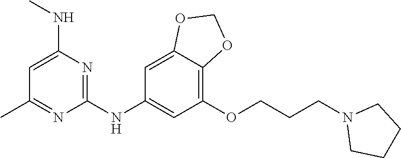 [SMILES: CNc1cc(C)nc(Nc2cc(OCCCN3CCCC3)c3c(c2)OCO3)n1] |
| 119 | | 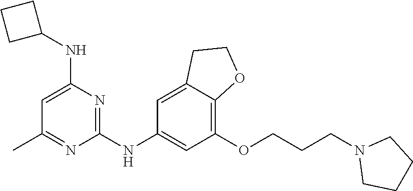 [SMILES: Cc1cc(NC2CCC2)nc(Nc2cc3c(c(OCCCN4CCCC4)c2)OCC3)n1] |
| 122 | | 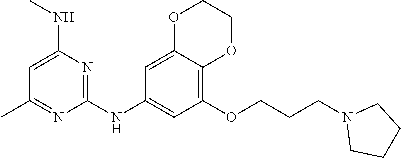 [SMILES: CNc1cc(C)nc(Nc2cc(OCCCN3CCCC3)c3c(c2)OCCO3)n1] |
| 124 | | 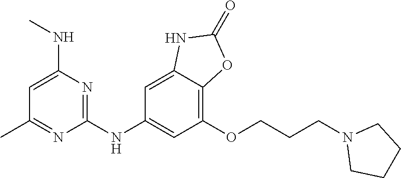 [SMILES: CNc1cc(C)nc(Nc2cc(OCCCN3CCCC3)c3oc(=O)[nH]c3c2)n1] |
| 125 | | 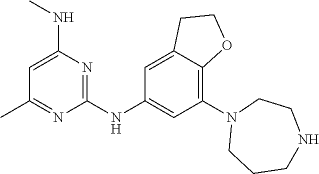 [SMILES: CNc1cc(C)nc(Nc2cc3c(c(N4CCCNCC4)c2)OCC3)n1] |
| 126 | | 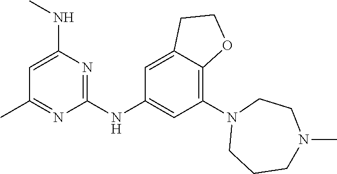 |
| 127 | | 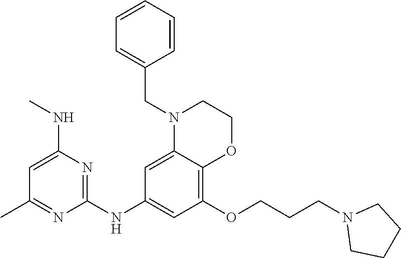 [SMILES: CNc1cc(C)nc(Nc2cc(OCCCN3CCCC3)c3c(c2)N(Cc2ccccc2)CCO3)n1] |
| 128 | (and) | 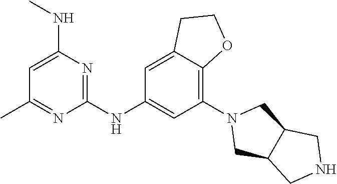 [SMILES: CNc1cc(C)nc(Nc2cc3c(c(N4C[C@H]5CNC[C@H]5C4)c2)OCC3)n1] |
| 129 | | 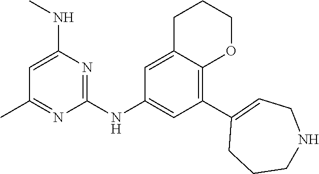 [SMILES: CNc1cc(C)nc(Nc2cc3c(c(C4=CCNCCC4)c2)OCCC3)n1] |
| 130 | | 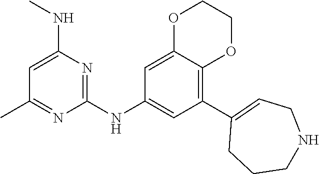 [SMILES: CNc1cc(C)nc(Nc2cc3c(c(C4=CCNCCC4)c2)OCCO3)n1] |
| 131 | | 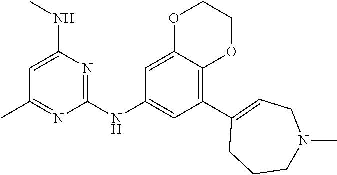 |
| 133 | (and) | 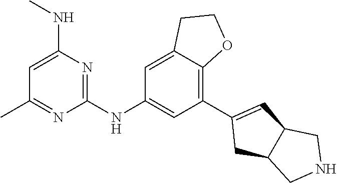 [SMILES: CNc1cc(C)nc(Nc2cc3c(c(C4=C[C@H]5CNC[C@H]5C4)c2)OCC3)n1] |
| 134 | (and) | 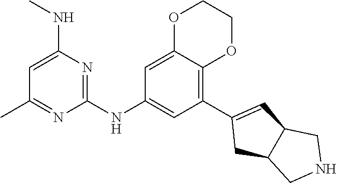 [SMILES: CNc1cc(C)nc(Nc2cc3c(c(C4=C[C@H]5CNC[C@H]5C4)c2)OCCO3)n1] |
| 136 | | 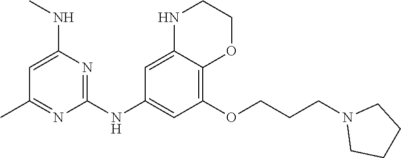 [SMILES: CNc1cc(C)nc(Nc2cc3c(c(OCCCN4CCCC4)c2)OCCN3)n1] |
| 137 | | 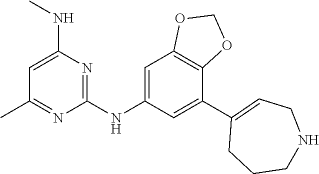 [SMILES: CNc1cc(C)nc(Nc2cc3c(c(C4=CCNCCC4)c2)OCO3)n1] |
| 138 | | 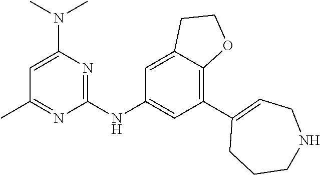 [SMILES: Cc1cc(N(C)C)nc(Nc2cc3c(c(C4=CCNCCC4)c2)OCC3)n1] |
| 139 | | 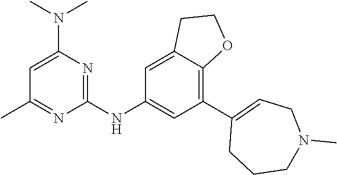 |
| 141 | | 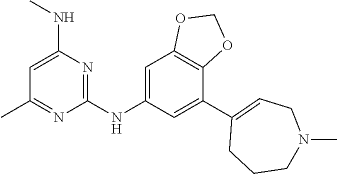 |
| 142 | | 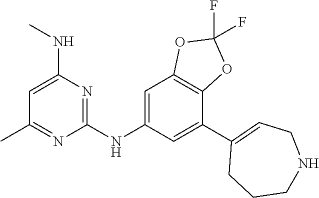 [SMILES: CNc1cc(C)nc(Nc2cc3c(c(C4=CCNCCC4)c2)OC(F)(F)O3)n1] |
| 143 | (and) | 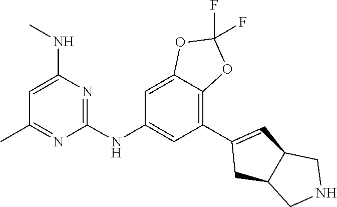 [SMILES: CNc1cc(C)nc(Nc2cc3c(c(C4=C[C@H]5CNC[C@H]5C4)c2)OC(F)(F)O3)n1] |
| 144 | | 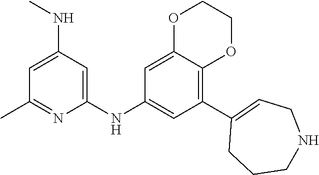 [SMILES: CNc1cc(C)nc(Nc2cc3c(c(C4=CCNCCC4)c2)OCCO3)c1] |
| 145 | | 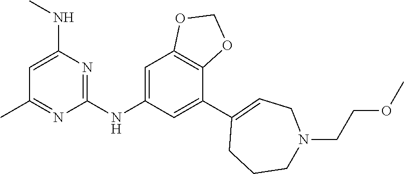 |
| 146 | | 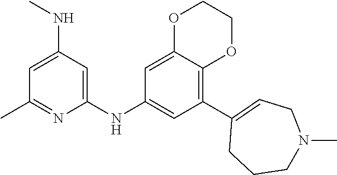 |
| 147 | | 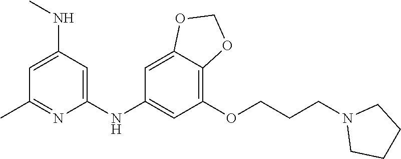 [SMILES: CNc1cc(C)nc(Nc2cc(OCCCN3CCCC3)c3c(c2)OCO3)c1] |
| 148 | | 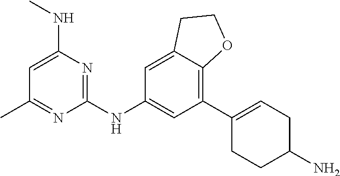 [SMILES: CNc1cc(C)nc(Nc2cc3c(c(C4=CCC(N)CC4)c2)OCC3)n1] |
| 149 | | 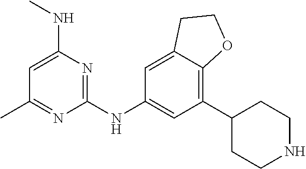 [SMILES: CNc1cc(C)nc(Nc2cc3c(c(C4CCNCC4)c2)OCC3)n1] |
| 150 | | 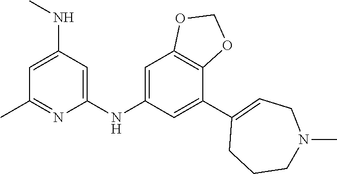 |
| 151 | | 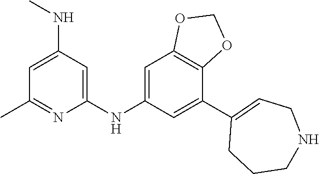 [SMILES: CNc1cc(C)nc(Nc2cc3c(c(C4=CCNCCC4)c2)OCO3)c1] |
| 152 | | 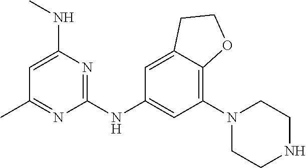 [SMILES: CNc1cc(C)nc(Nc2cc3c(c(N4CCNCC4)c2)OCC3)n1] |
| 153 | | 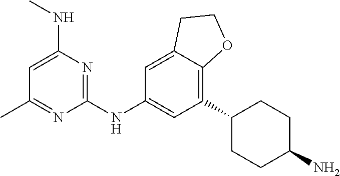 [SMILES: CNc1cc(C)nc(Nc2cc3c(c([C@H]4CC[C@H](N)CC4)c2)OCC3)n1] |
| 154 | | 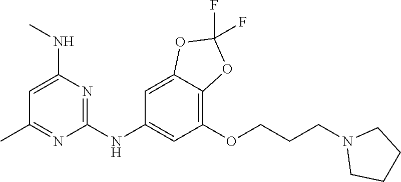 [SMILES: CNc1cc(C)nc(Nc2cc(OCCCN3CCCC3)c3c(c2)OC(F)(F)O3)n1] |
| 155 | | 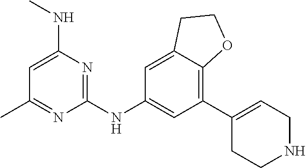 [SMILES: CNc1cc(C)nc(Nc2cc3c(c(C4=CCNCC4)c2)OCC3)n1] |
| 156 | | 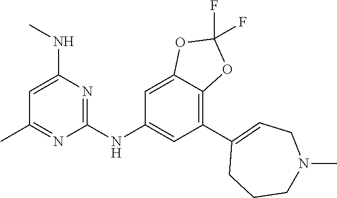 |
| 157 | (and) | 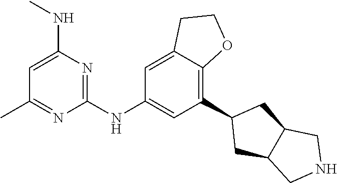 [SMILES: CNc1cc(C)nc(Nc2cc3c(c([C@H]4C[C@H]5CNC[C@H]5C4)c2)OCC3)n1] |
| 158 | | 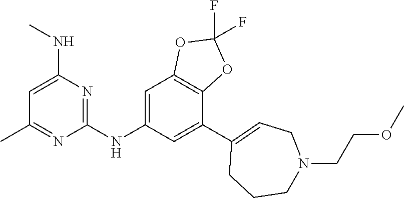 |
| 159 | (or) | 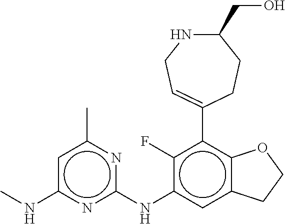 |
| 159-a | (or) | 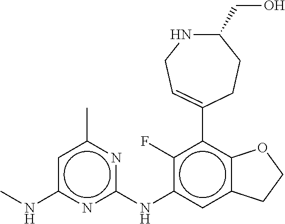 |
| 160 | | 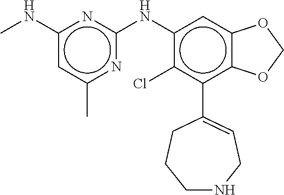 |
| 161 | (or) | 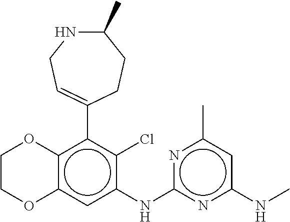 |
| 161-a | (or) | 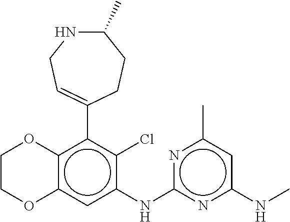 |
| 162 | (or) | 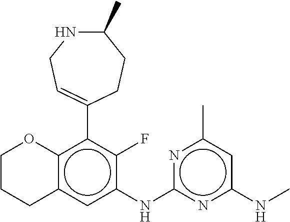 |
| 163 | (or) | 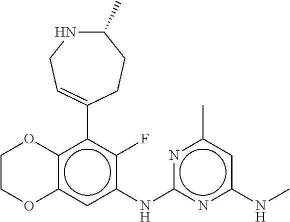 |
| 163-a | (or) | 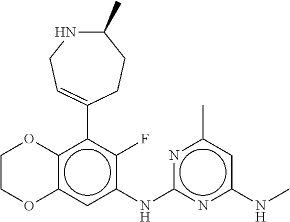 |
| 164 | (or) | 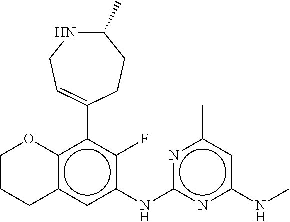 |
| 165 | | 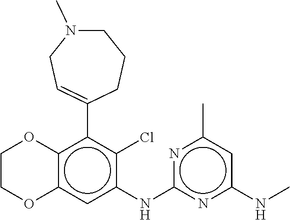 |
| 166 | | 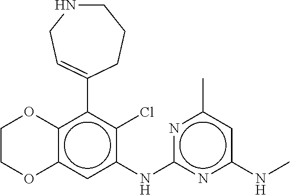 |
| 167 | (or) | 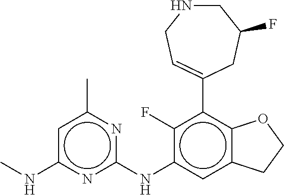 |
| 168 | (or) | 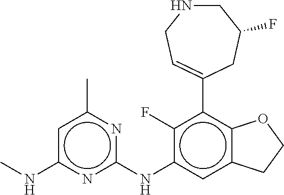 |
| | | B |
| 169 | (or) | 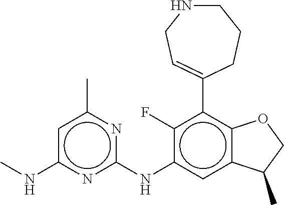 |
| 170 | (or) | 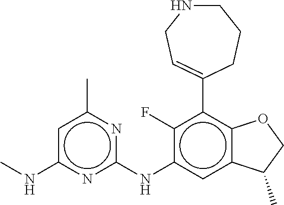 |
| 171 | | 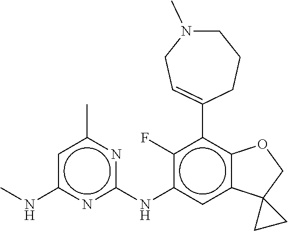 |
| 172 | | 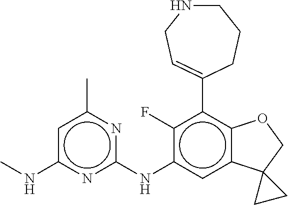 |
| 173 | | 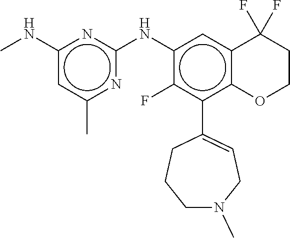 |
| 174 | | 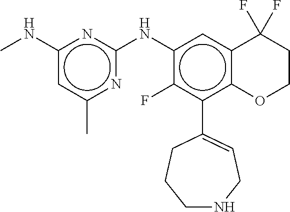 |
| 175 | | 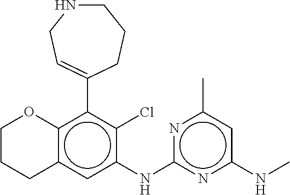 |
| 176 | | 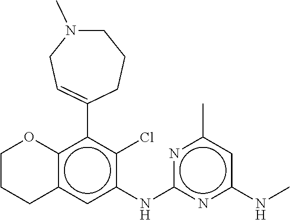 |
| 178 | | 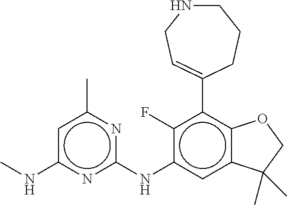 |
| 179 | | 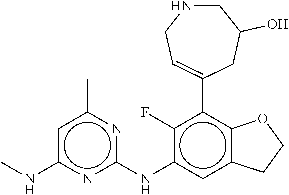 |
| 180 | | 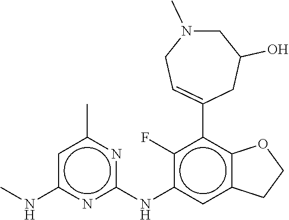 |
| 181 | | 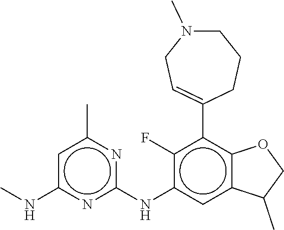 |
| 182 | (or) | 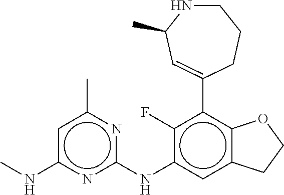 |
| 183 | (or) | 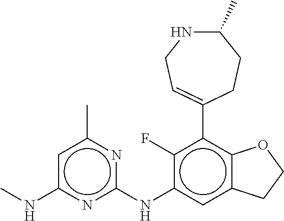 |
| 184 | (or) | 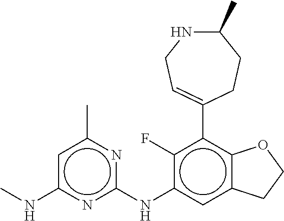 |
| 185 | (or) | 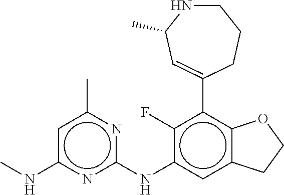 |
| 186 | | 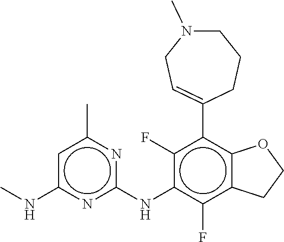 |
| 187 | | 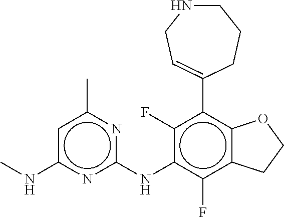 |
| 188 | | 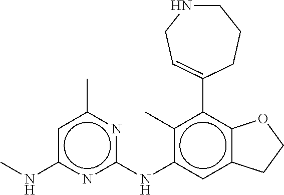 |
| 189 | | 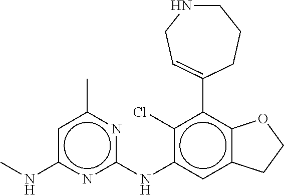 |
| 190 | | 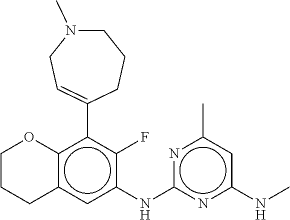 |
| 191 | | 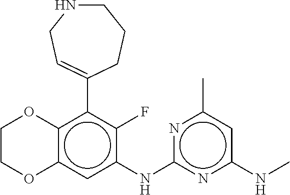 |
| 192 | | 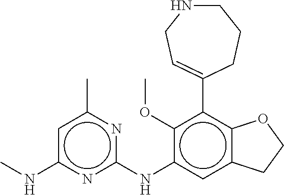 |
| 193 | | 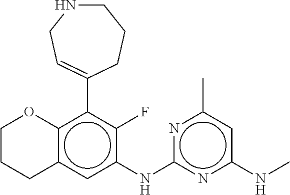 |
| 194 | | 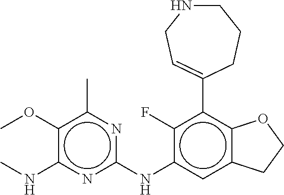 |
| 195 | | 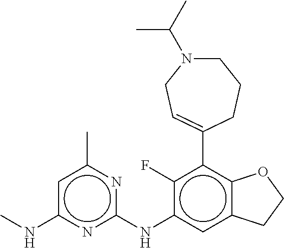 |
| 196 | | 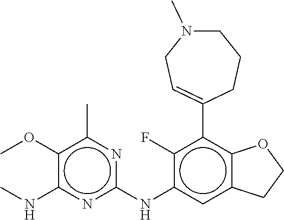 |
| 197 | | 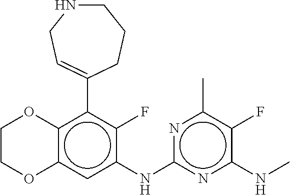 |
| 198 | | 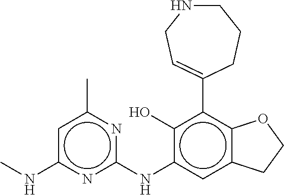 |
| 199 | | 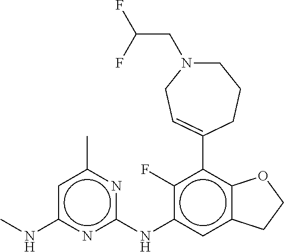 |
| 200 | | 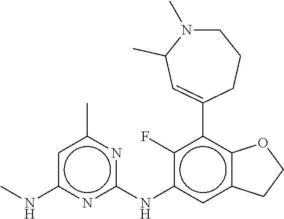 |
| 201 | | 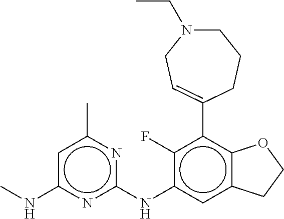 |
| 202 | | 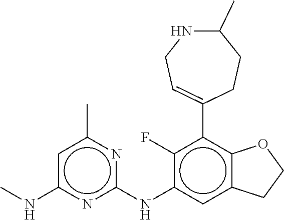 |
| 203 | | 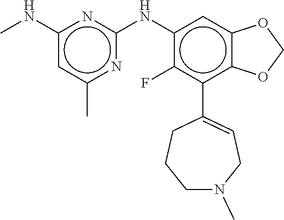 |
| 204 | | 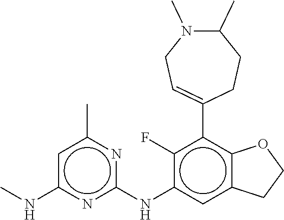 |
| 205 | | 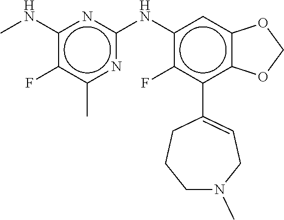 |
| 206 | | 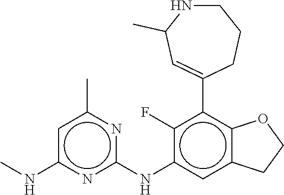 |
| 207 | | 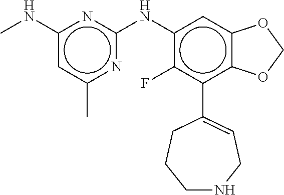 |
| 208 | | 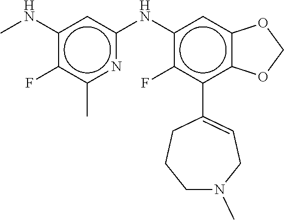 |
| 209 | | 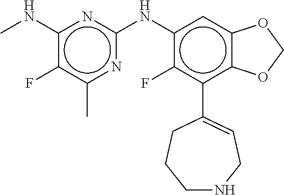 |
| 210 | | 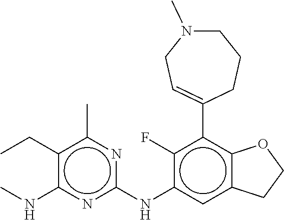 |
| 211 | | 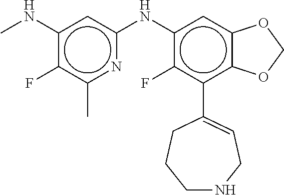 |
| 212 | | 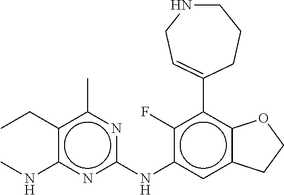 |
| 214 | | 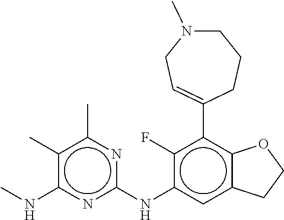 |
| 215 | | 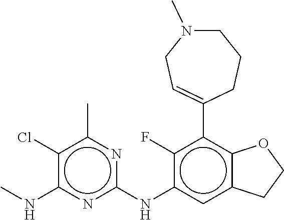 |
| 216 | | 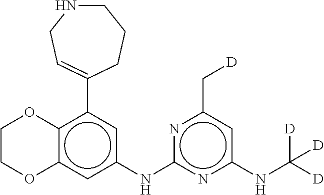 |
| 217 | | 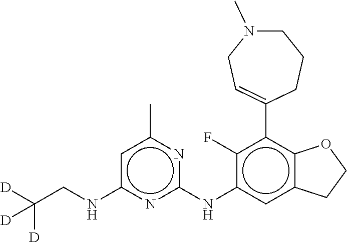 |
| 218 | | 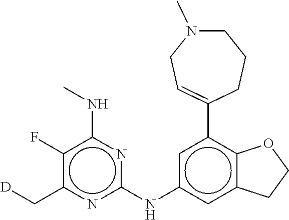 |
| 219 | | 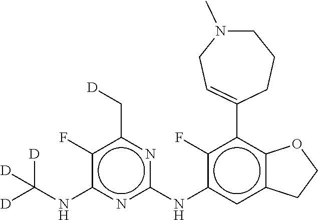 |
| 220 | | 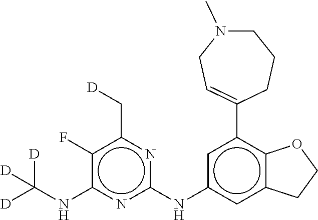 |
| 221 | | 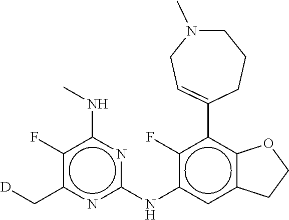 |
| 222 | | 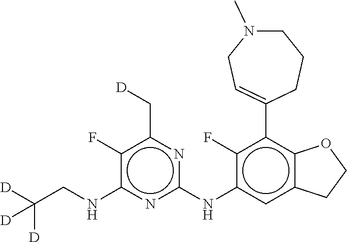 |
| 223 | | 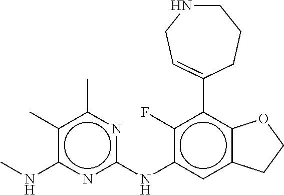 |
| 224 | | 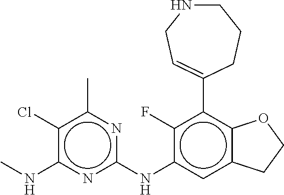 |
| 225 | | 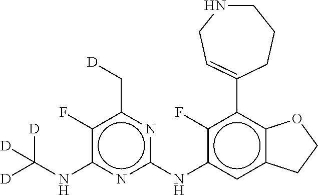 |
| 226 | | 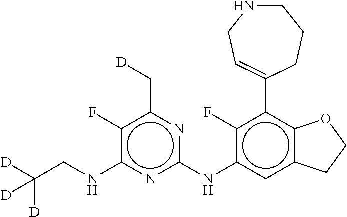 |
| 227 | | 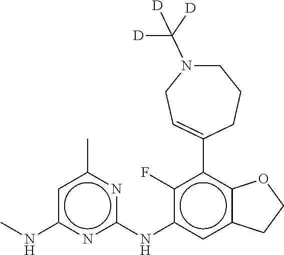 |
| 228 | | 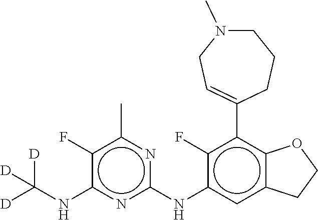 |
| 229 | | 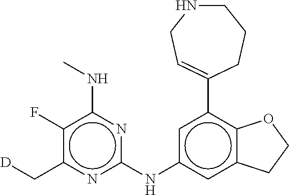 |
| 230 | | 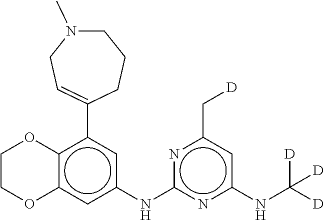 |
| 231 | | 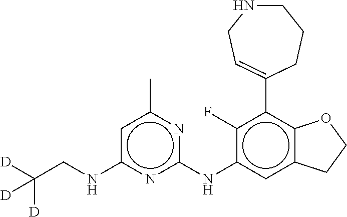 |
| 232 | | 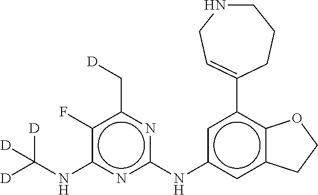 |
| 233 | | 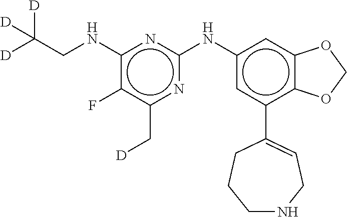 |
| 234 | | 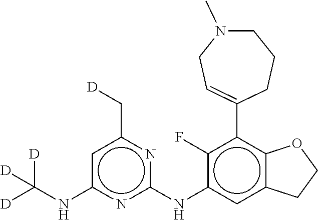 |
| 235 | | 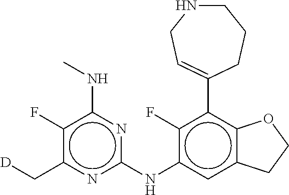 |
| 236 | | 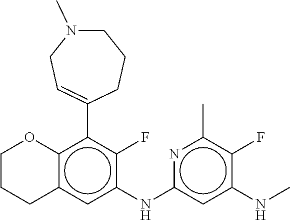 |
| 237 | | 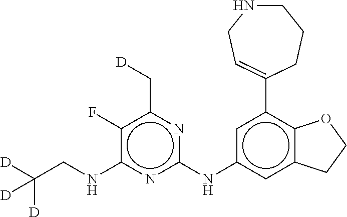 |
| 238 | | 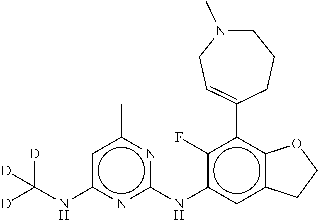 |
| 239 | | 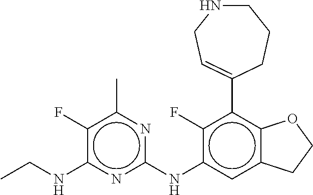 |
| 240 | | 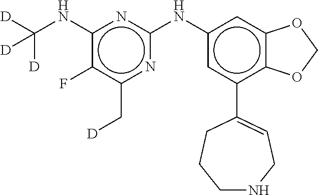 |
| 241 | | 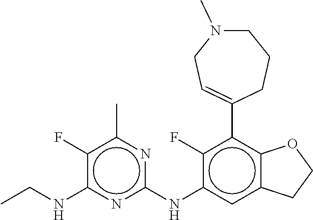 |
| 242 | | 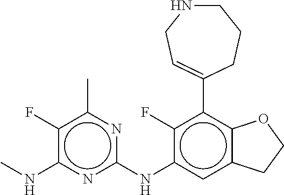 |
| 243 | | 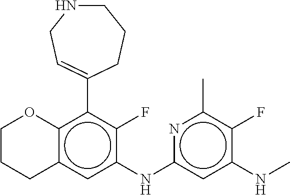 |
| 244 | | 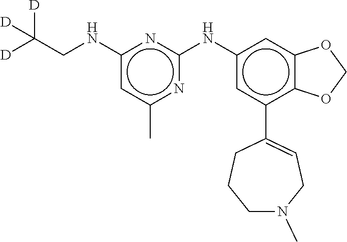 |
| 245 | | 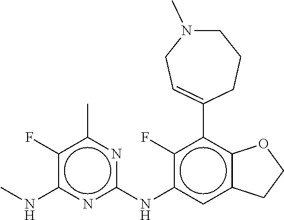 |
| 246 | | 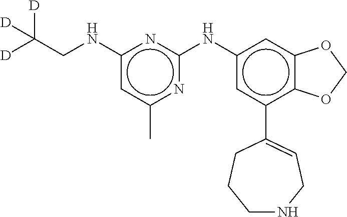 |
| 247 | | 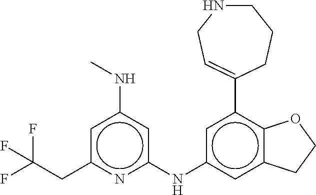 |
| 248 | | 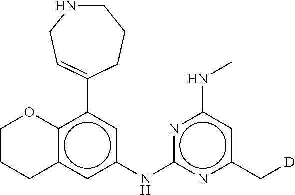 |
| 249 | | 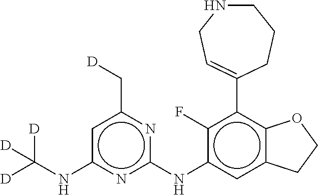 |
| 250 | | 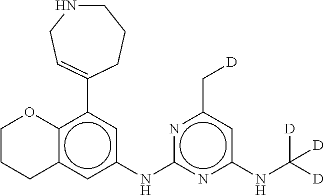 |
| 251 | | 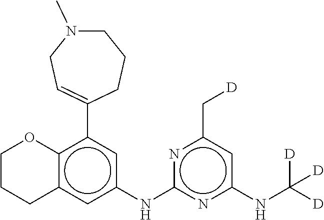 |
| 252 | | 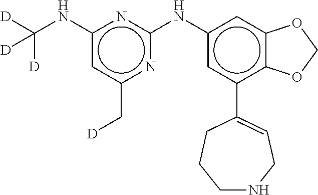 |
| 253 | | 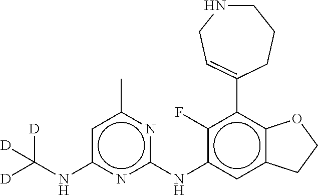 |
| 254 | | 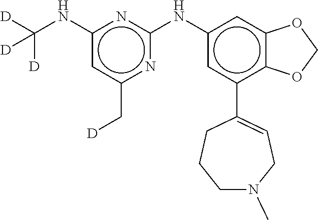 |
| 255 | | 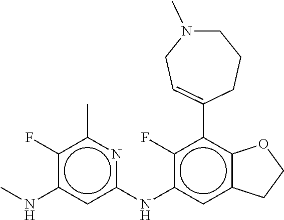 |
| 256 | | 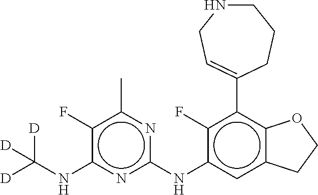 |
| 257 | | 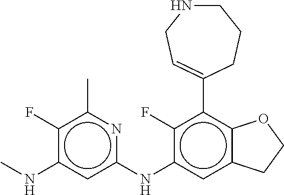 |
| 258 | | 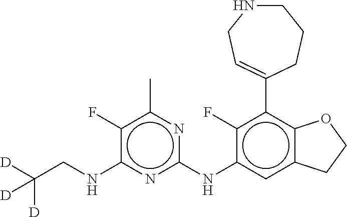 |
| 259 | | 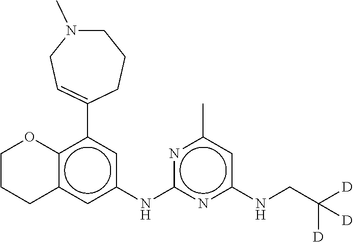 |
| 260 | | 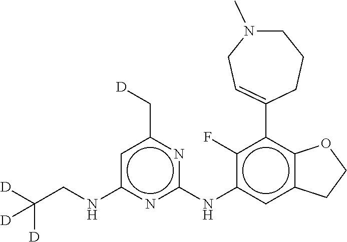 |
| 261 | | 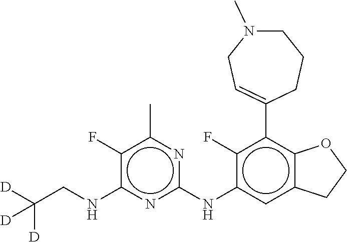 |
| 262 | | 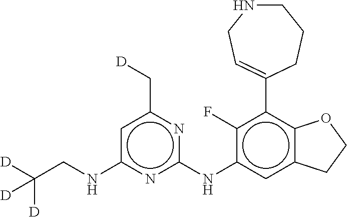 |
| 263 | | 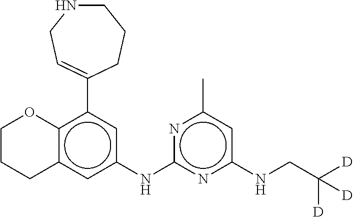 |
| 264 | | 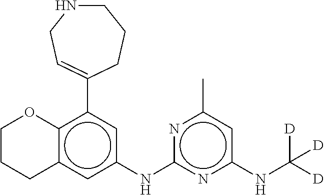 |
| 265 | | 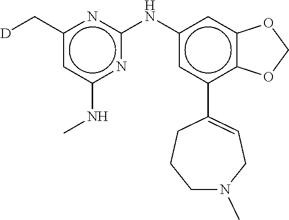 |
| 266 | | 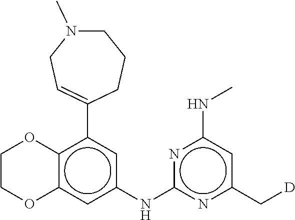 |
| 267 | | 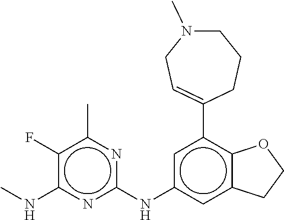 |
| 268 | | 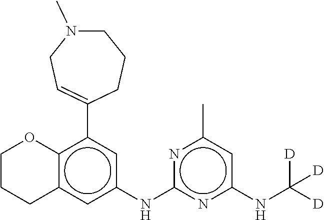 |
| 269 | | 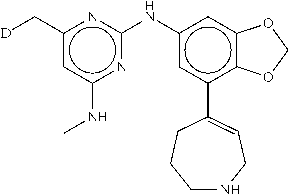 |
| 270 | | 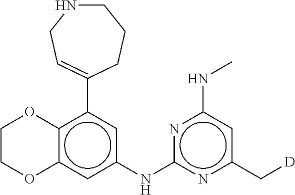 |
| 271 | | 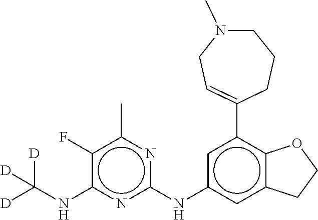 |
| 272 | | 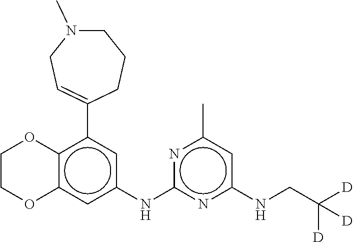 |
| 273 | | 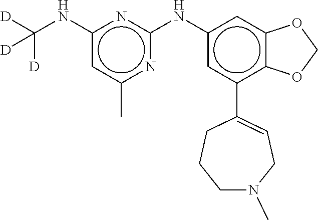 |
| 274 | | 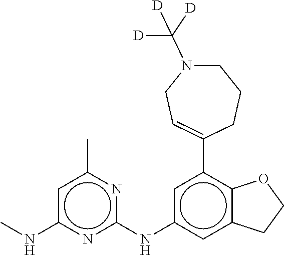 |
| 275 | | 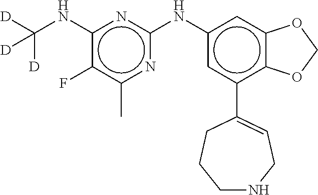 |
| 276 | | 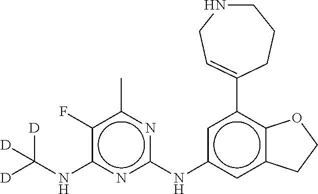 |
| 277 | | 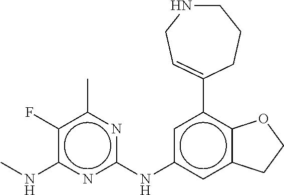 |
| 278 | | 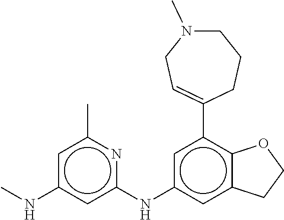 |
| 279 | | 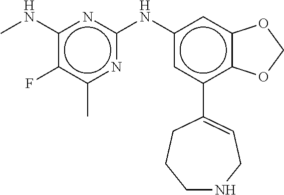 |
| 280 | | 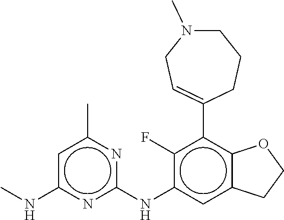 |
| 281 | | 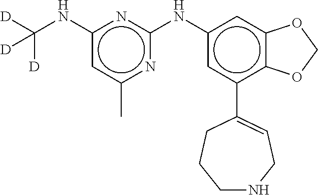 |
| 282 | | 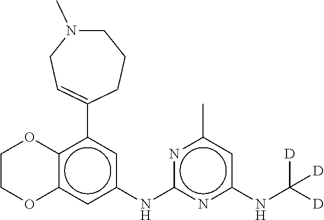 |
| 283 | | 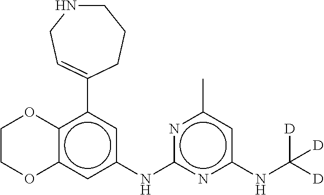 |
| 284 | | 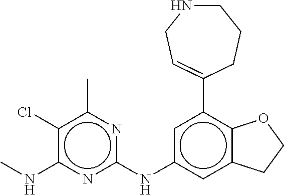 |
| 285 | | 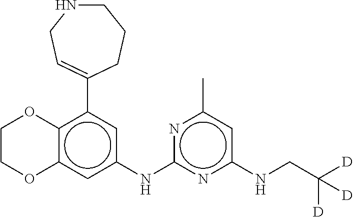 |
| 286 | | 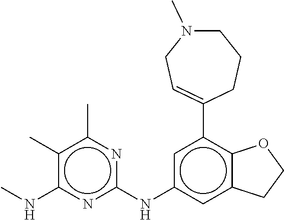 |
| 287 | | 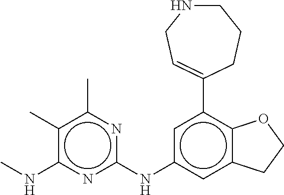 |
| 288 | | 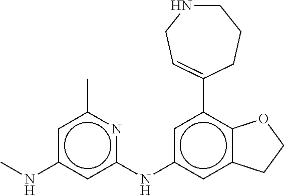 |
| 289 | | 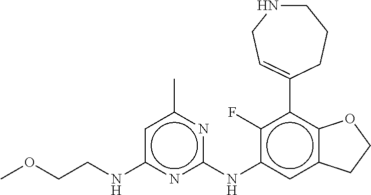 |
| 290 | | 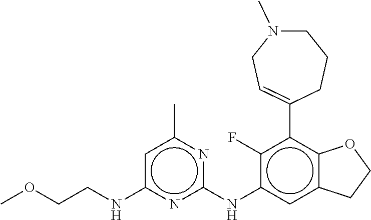 |
| 291 | | 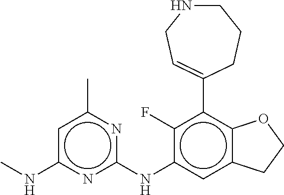 |
| 292 | | 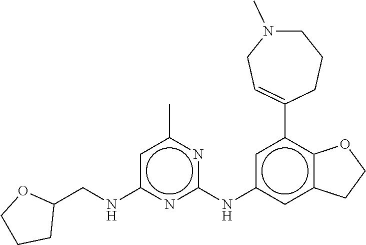 |
| 293 | | 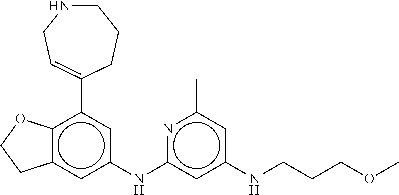 |
| 294 | | 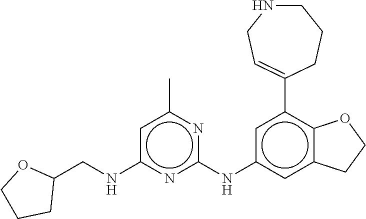 |
| 295 | | 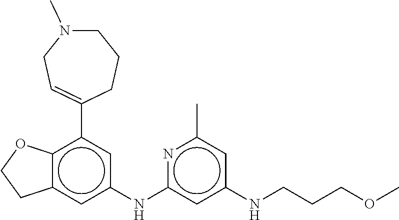 |
| 296 | | 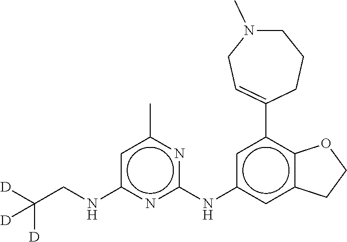 |
| 297 | | 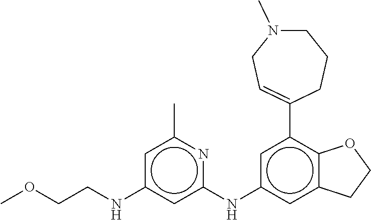 |
| 298 | | 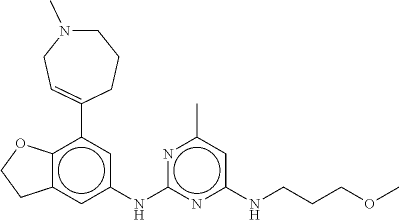 |
| 299 | | 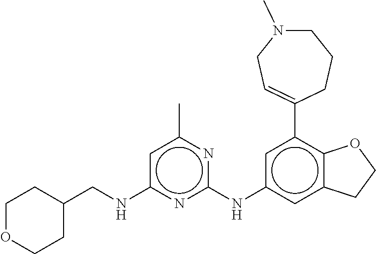 |
| 300 | | 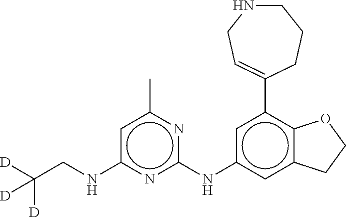 |
| 301 | | 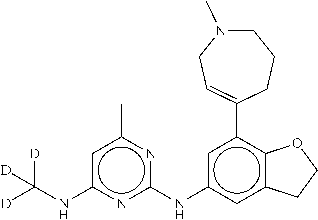 |
| 302 | | 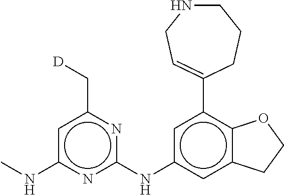 |
| 303 | | 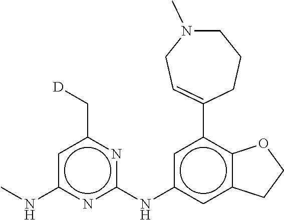 |
| 304 | | 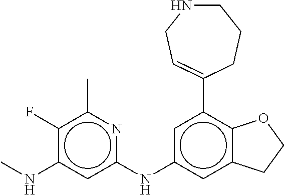 |
| 305 | | 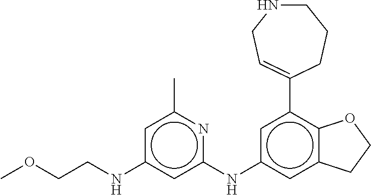 |
| 306 | | 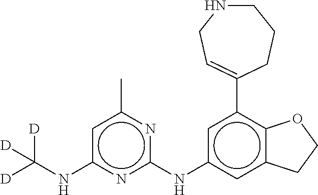 |
| 307 | | 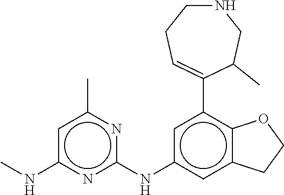 |
| 308 | | 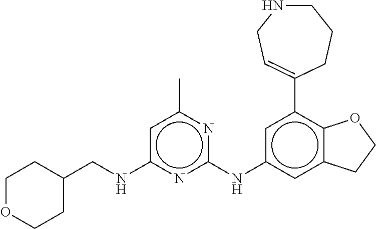 |
| 309 | | 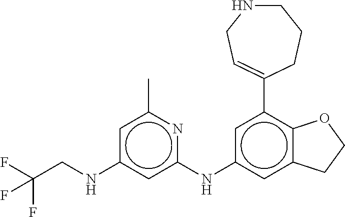 |
| 310 | | 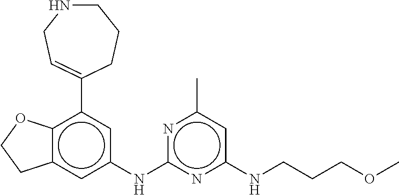 |
| 311 | | 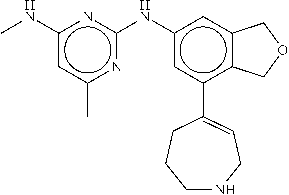 |
| 312 | | 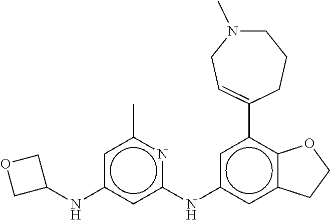 |
| 313 | | 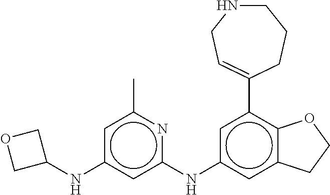 |
| 314 | | 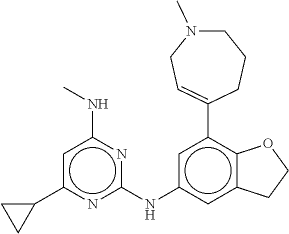 |
| 315 | | 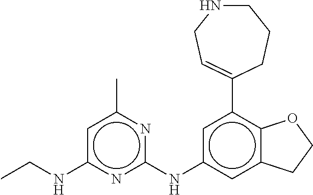 |
| 316 | | 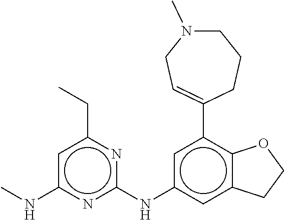 |
| 317 | | 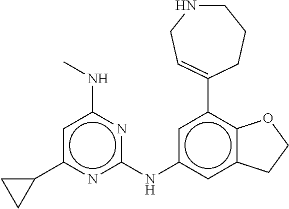 |
| 318 | | 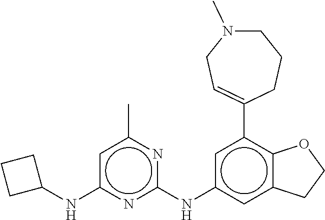 |
| 319 | | 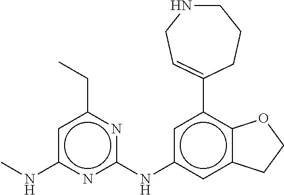 |
| 320 | | 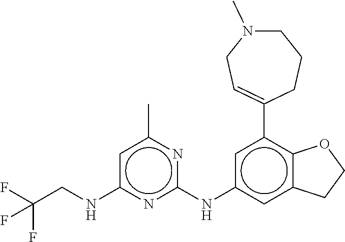 |
| 321 | | 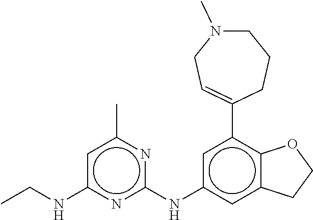 |
| 322 | | 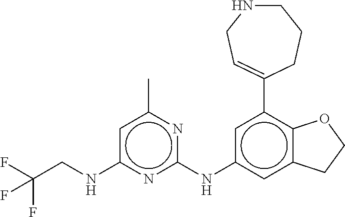 |
| 323 | | 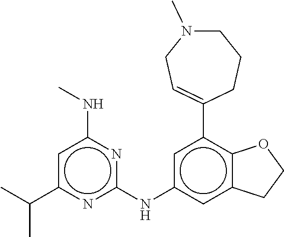 |
| 324 | | 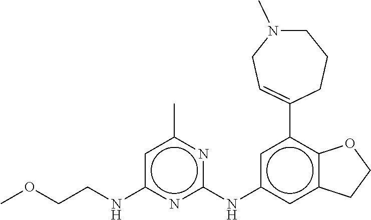 |
| 325 | | 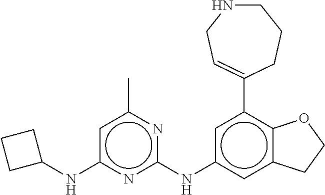 |
| 326 | | 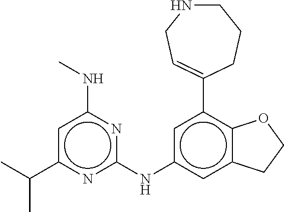 |
| 327 | | 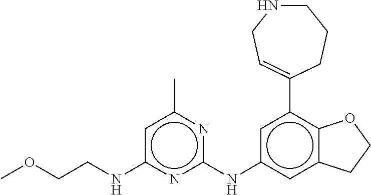 |
| 328 | | 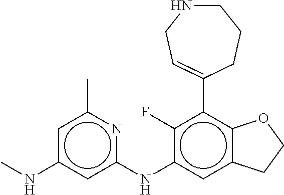 |
| 329 | (and) | 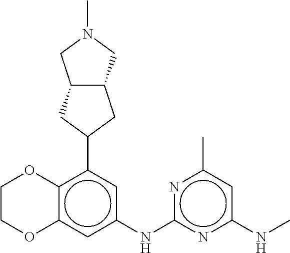 |
| 330 | | 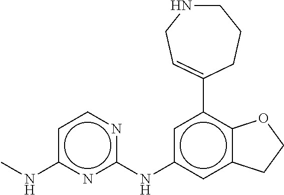 |
| 331 | | 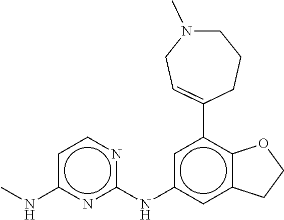 |
| 332 | | 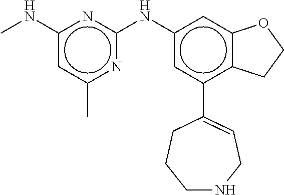 |
| 333 | | 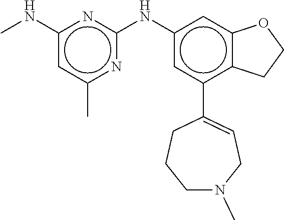 |
| 334 | (and) | 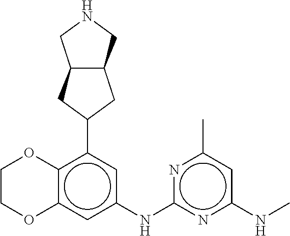 |
| 335 | | 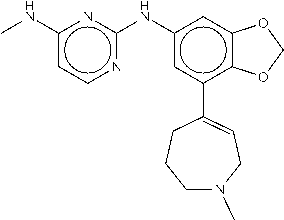 |
| 336 | | 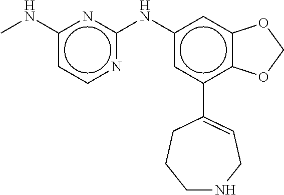 |
| 337 | (and) | 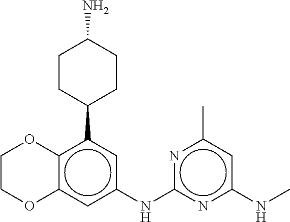 |
| 338 | (and) | 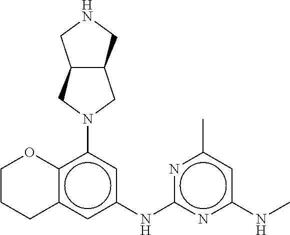 |
| 339 | | 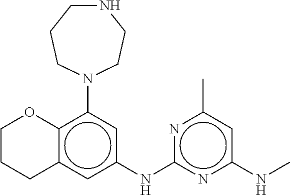 |
| 340 | (and) | 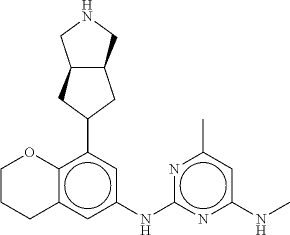 |
| 341 | | 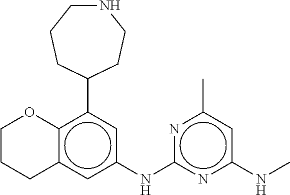 |
| 342 | (and) | 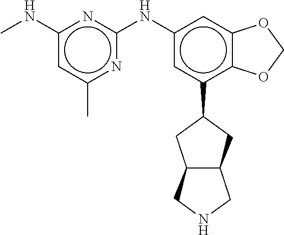 |
| 343 | | 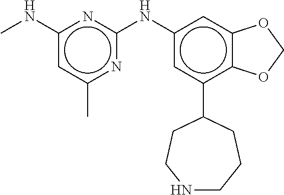 |
| 344 | (and) | 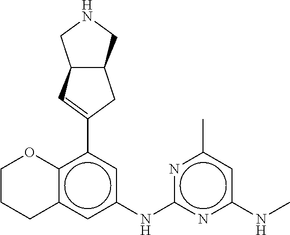 |
| 345 | | 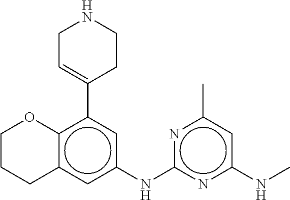 |
| 346 | | 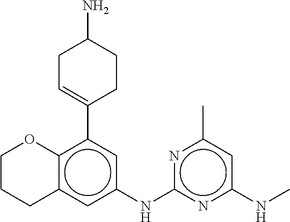 |
| 347 | (and) | 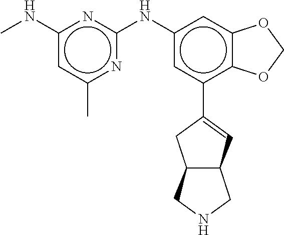 |
| 348 | | 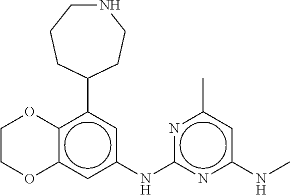 |
| 351 | (and) | 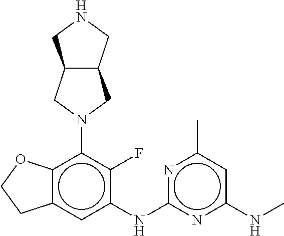 |
| 352 | (or) | 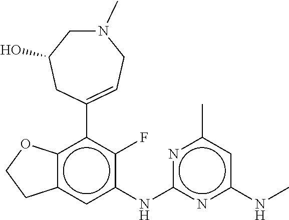 |
| 353 | | 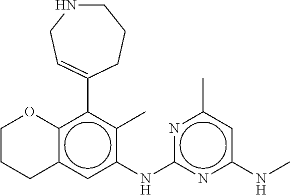 |
| 354 | (or) | 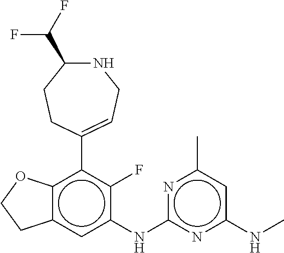 |
| 354-a | (or) | 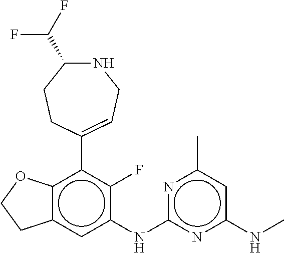 |
| 355 | (or) | 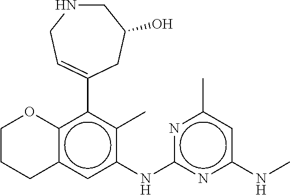 |
| 356 | (or) | 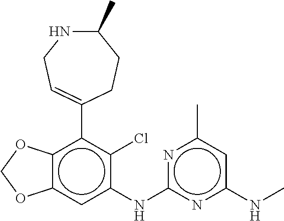 |
| 356-a | (or) | 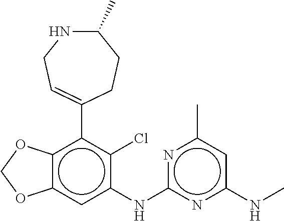 |
| 357 | (or) | 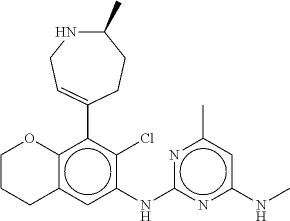 |
| 358 | (or) | 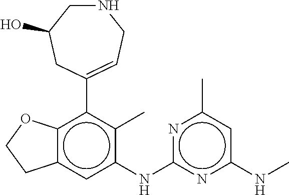 |
| 359 | (and) | 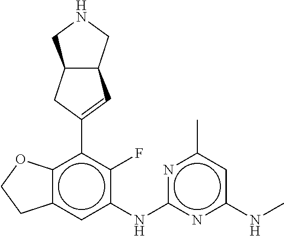 |
| 360 | (or) | 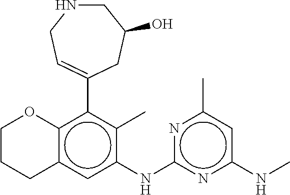 |
| 363 | | 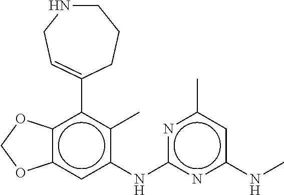 |
| 365 | (or) | 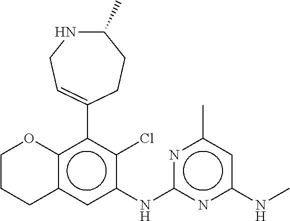 |
| 366 | (or) | 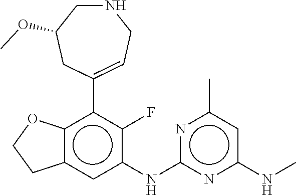 |
| 366-a | (or) | 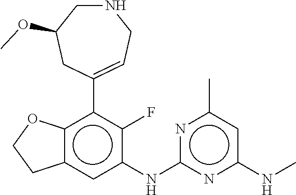 |
| 368 | (or) | 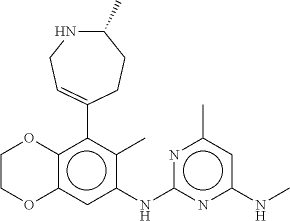 |
| 368-a | (or) | 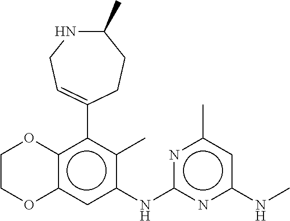 |
| 369 | (or) | 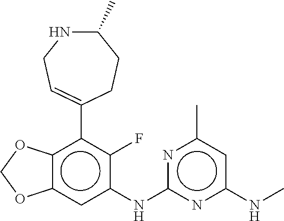 |
| 370 | (or) | 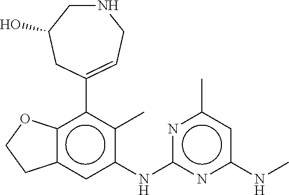 |
| 371 | (or) | 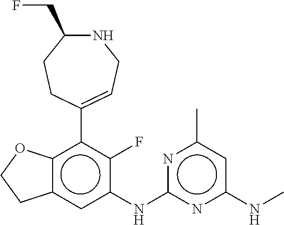 |
| 371-a | (or) | 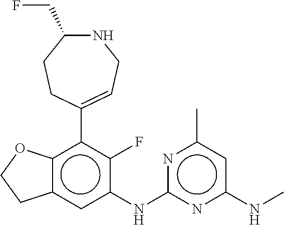 |
| 373 | (or) | 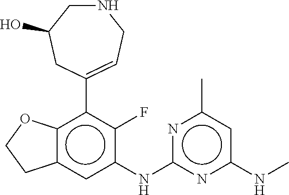 |
| 374 | (or) | 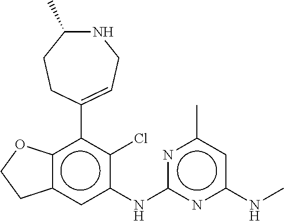 |
| 374-a | (or) | 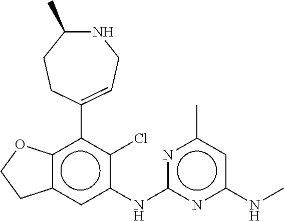 |
| 375 | (or) | 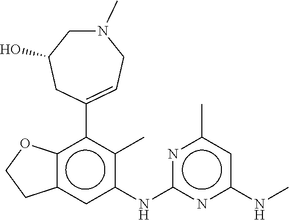 |
| 376 | (or) | 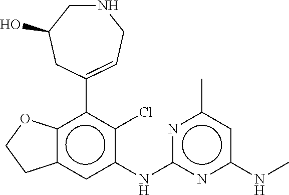 |
| 379 | (or) | 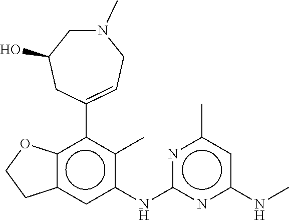 |
| 380 | (or) | 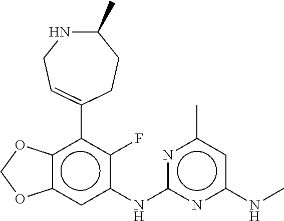 |
| 381 | (or) | 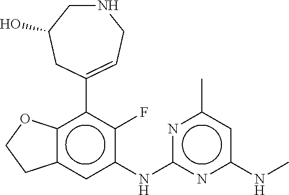 |
| 382 | (or) | 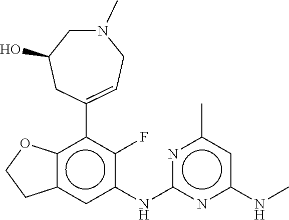 |
| 383 | (or) | 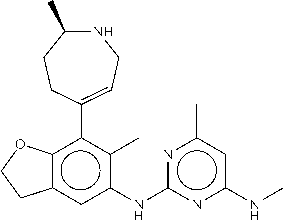 |
| 383-a | (or) | 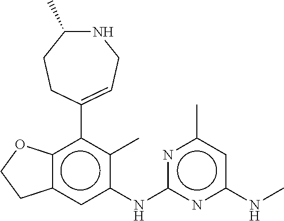 |
| 384 | (or) | 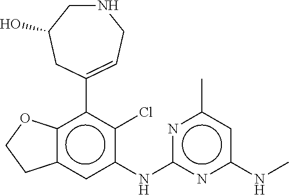 |
| 385 | (or) | 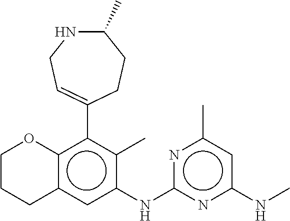 |
| 385-a | (or) | 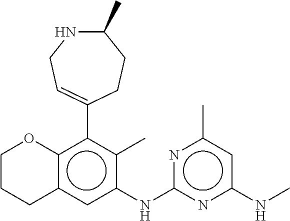 |
| 386 | (or) | 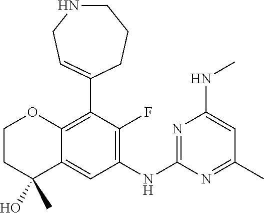 [SMILES: CNc1cc(C)nc(Nc2cc3c(c(C4=CCNCCC4)c2F)OCC[C@@]3(C)O)n1] |
| 387 | (or) | 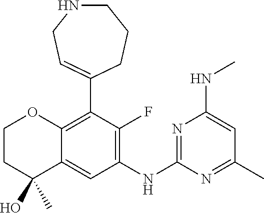 [SMILES: CNc1cc(C)nc(Nc2cc3c(c(C4=CCNCCC4)c2F)OCC[C@]3(C)O)n1] |
| 388(*) | (or) | 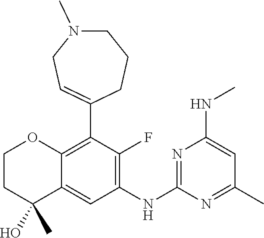 [SMILES: CNc1cc(C)nc(Nc2cc3c(c(C4=CCN(C)CCC4)c2F)OCC[C@@]3(C)O)n1] |
| 389 | | 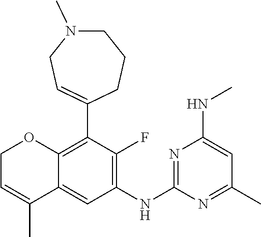 [SMILES: CNc1cc(C)nc(Nc2cc3c(c(C4=CCN(C)CCC4)c2F)OCC=C3C)n1] |
| 390(*) | (or) | 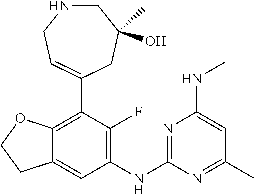 [SMILES: CNc1cc(C)nc(Nc2cc3c(c(C4=CCNC[C@@](C)(O)C4)c2F)OCC3)n1] |
| 391(*) | (or) | 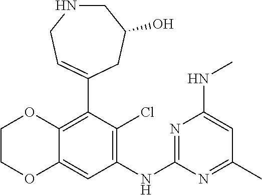 [SMILES: CNc1cc(C)nc(Nc2cc3c(c(C4=CCNC[C@H](O)C4)c2Cl)OCCO3)n1] |
| 392(*) | (or) | 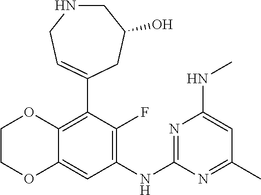 [SMILES: CNc1cc(C)nc(Nc2cc3c(c(C4=CCNC[C@H](O)C4)c2F)OCCO3)n1] |
| 393(*) | (or) | 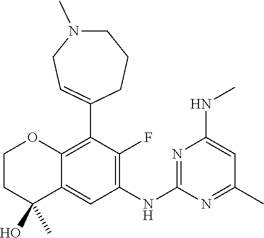 |
| 394(*) | (or) | 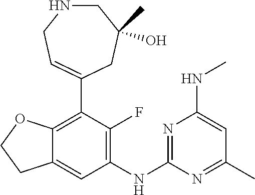 [SMILES: CNc1cc(C)nc(Nc2cc3c(c(C4=CCNC[C@](C)(O)C4)c2F)OCC3)n1] |
| 395(*) | (or) | 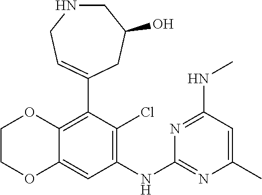 [SMILES: CNc1cc(C)nc(Nc2cc3c(c(C4=CCNC[C@@H](O)C4)c2Cl)OCCO3)n1] |
| 396(*) | (or) | 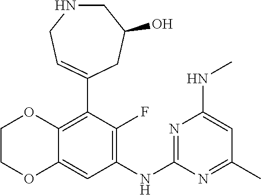 [SMILES: CNc1cc(C)nc(Nc2cc3c(c(C4=CCNC[C@@H](O)C4)c2F)OCCO3)n1] |
| 397 | (or) | 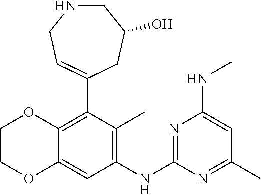 [SMILES: CNc1cc(C)nc(Nc2cc3c(c(C4=CCNC[C@H](O)C4)c2C)OCCO3)n1] |
| 398 | (or) | 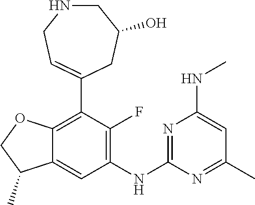 [SMILES: CNc1cc(C)nc(Nc2cc3c(c(C4=CCNC[C@H](O)C4)c2F)OC[C@H]3C)n1] |
| 399 | (or) | 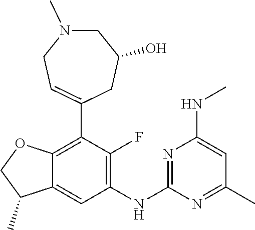 [SMILES: CNc1cc(C)nc(Nc2cc3c(c(C4=CCN(C)C[C@H](O)C4)c2F)OC[C@H]3C)n1] |
| 400 | (or) | 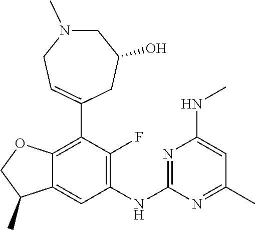 [SMILES: CNc1cc(C)nc(Nc2cc3c(c(C4=CCN(C)C[C@H](O)C4)c2F)OC[C@@H]3C)n1] |
| 401 | (or) | 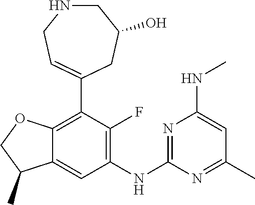 [SMILES: CNc1cc(C)nc(Nc2cc3c(c(C4=CCNC[C@H](O)C4)c2F)OC[C@@H]3C)n1] |
| 402 | (or) | 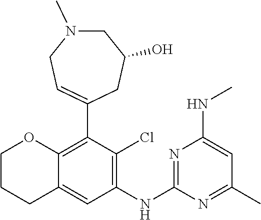 [SMILES: CNc1cc(C)nc(Nc2cc3c(c(C4=CCN(C)C[C@H](O)C4)c2Cl)OCCC3)n1] |
| 403 | (or) | 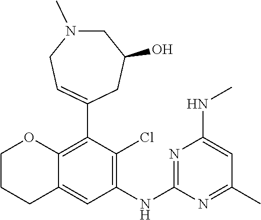 [SMILES: CNc1cc(C)nc(Nc2cc3c(c(C4=CCN(C)C[C@@H](O)C4)c2Cl)OCCC3)n1] |
| 404(*) | (or) | 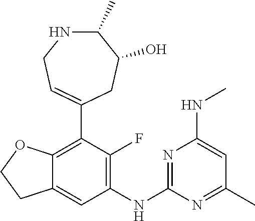 [SMILES: CNc1cc(C)nc(Nc2cc3c(c(C4=CCN[C@H](C)[C@H](O)C4)c2F)OCC3)n1] |
| 405(*) | (or) | 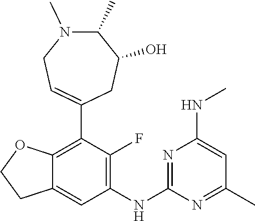 [SMILES: CNc1cc(C)nc(Nc2cc3c(c(C4=CCN(C)[C@H](C)[C@H](O)C4)c2F)OCC3)n1] |
| 406 | (or) | 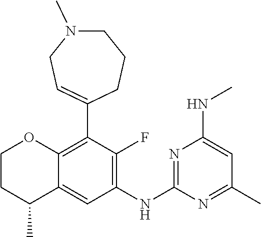 [SMILES: CNc1cc(C)nc(Nc2cc3c(c(C4=CCN(C)CCC4)c2F)OCC[C@H]3C)n1] |
| 407 | (or) | 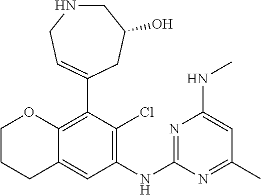 [SMILES: CNc1cc(C)nc(Nc2cc3c(c(C4=CCNC[C@H](O)C4)c2Cl)OCCC3)n1] |
| 408 | (or) | 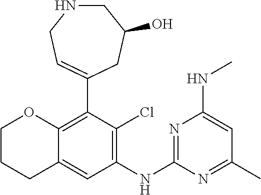 [SMILES: CNc1cc(C)nc(Nc2cc3c(c(C4=CCNC[C@@H](O)C4)c2Cl)OCCC3)n1] |
| 409 | (or) | 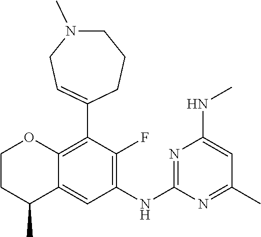 [SMILES: CNc1cc(C)nc(Nc2cc3c(c(C4=CCN(C)CCC4)c2F)OCC[C@@H]3C)n1] |
| 410(*) | (or) | 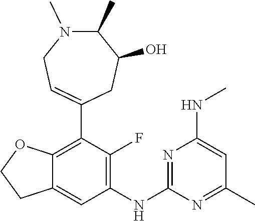 [SMILES: CNc1cc(C)nc(Nc2cc3c(c(C4=CCN(C)[C@@H](C)[C@@H](O)C4)c2F)OCC3)n1] |
| 411(*) | (or) | 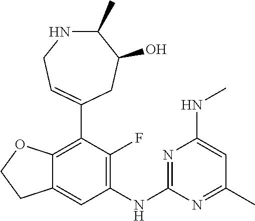 [SMILES: CNc1cc(C)nc(Nc2cc3c(c(C4=CCN[C@@H](C)[C@@H](O)C4)c2F)OCC3)n1] |
| 412(*) | (or) | 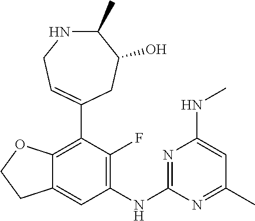 [SMILES: CNc1cc(C)nc(Nc2cc3c(c(C4=CCN[C@@H](C)[C@H](O)C4)c2F)OCC3)n1] |
| 413(*) | (or) | 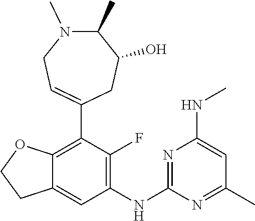 [SMILES: CNc1cc(C)nc(Nc2cc3c(c(C4=CCN(C)[C@@H](C)[C@H](O)C4)c2F)OCC3)n1] |
| 414 | (or) | 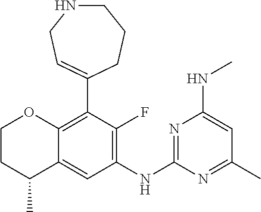 [SMILES: CNc1cc(C)nc(Nc2cc3c(c(C4=CCNCCC4)c2F)OCC[C@H]3C)n1] |
| 415 | | 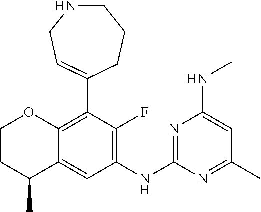 [SMILES: CNc1cc(C)nc(Nc2cc3c(c(C4=CCNCCC4)c2F)OCC[C@@H]3C)n1] |
| 416(*) | (or) | 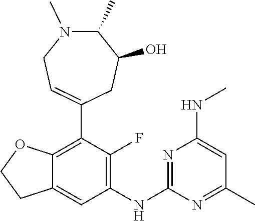 [SMILES: CNc1cc(C)nc(Nc2cc3c(c(C4=CCN(C)[C@H](C)[C@@H](O)C4)c2F)OCC3)n1] |
| 417(*) | (or) | 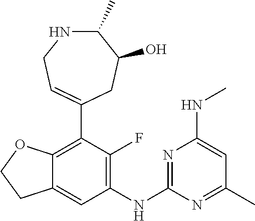 [SMILES: CNc1cc(C)nc(Nc2cc3c(c(C4=CCN[C@H](C)[C@@H](O)C4)c2F)OCC3)n1] |
| 418 | (or) | 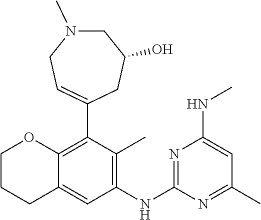 [SMILES: CNc1cc(C)nc(Nc2cc3c(c(C4=CCN(C)C[C@H](O)C4)c2C)OCCC3)n1] |
| 419 | (or) | 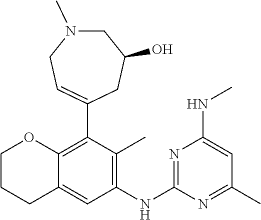 [SMILES: CNc1cc(C)nc(Nc2cc3c(c(C4=CCN(C)C[C@@H](O)C4)c2C)OCCC3)n1] |
| 420 | (or) | 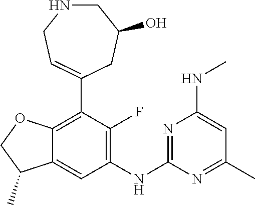 [SMILES: CNc1cc(C)nc(Nc2cc3c(c(C4=CCNC[C@@H](O)C4)c2F)OC[C@H]3C)n1] |
| 421 | (or) | 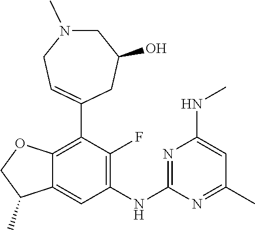 [SMILES: CNc1cc(C)nc(Nc2cc3c(c(C4=CCN(C)C[C@@H](O)C4)c2F)OC[C@H]3C)n1] |
| 422 | (or) | 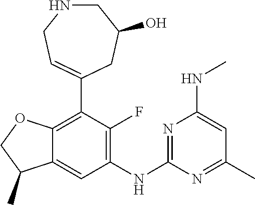 [SMILES: CNc1cc(C)nc(Nc2cc3c(c(C4=CCNC[C@@H](O)C4)c2F)OC[C@@H]3C)n1] |
| 423 | (or) | 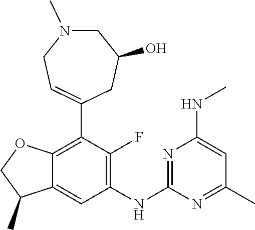 [SMILES: CNc1cc(C)nc(Nc2cc3c(c(C4=CCN(C)C[C@@H](O)C4)c2F)OC[C@@H]3C)n1] |
| 424(*) | (or) | 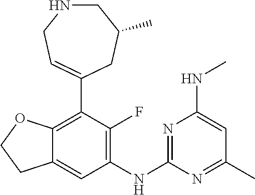 [SMILES: CNc1cc(C)nc(Nc2cc3c(c(C4=CCNC[C@H](C)C4)c2F)OCC3)n1] |
| 425(*) | (or) | 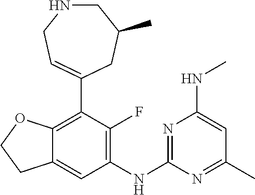 [SMILES: CNc1cc(C)nc(Nc2cc3c(c(C4=CCNC[C@@H](C)C4)c2F)OCC3)n1] |
| 426 | (or) | 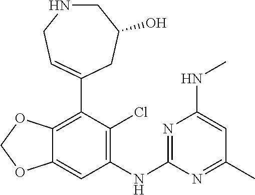 [SMILES: CNc1cc(C)nc(Nc2cc3c(c(C4=CCNC[C@H](O)C4)c2Cl)OCO3)n1] |
| 427 | (or) | 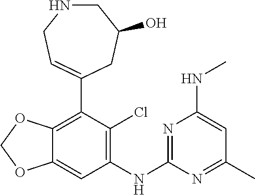 [SMILES: CNc1cc(C)nc(Nc2cc3c(c(C4=CCNC[C@@H](O)C4)c2Cl)OCO3)n1] |
| 428 | (or) | 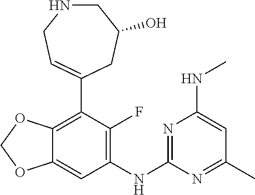 [SMILES: CNc1cc(C)nc(Nc2cc3c(c(C4=CCNC[C@H](O)C4)c2F)OCO3)n1] |
| 429 | (or) | 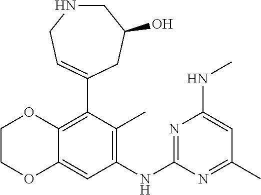 [SMILES: CNc1cc(C)nc(Nc2cc3c(c(C4=CCNC[C@@H](O)C4)c2C)OCCO3)n1] |
| 430 | (or) | 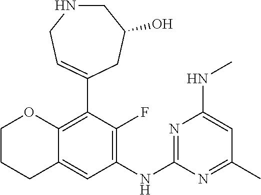 [SMILES: CNc1cc(C)nc(Nc2cc3c(c(C4=CCNC[C@H](O)C4)c2F)OCCC3)n1] |
| 431(*) | (or) | 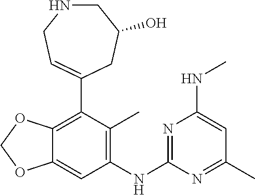 [SMILES: CNc1cc(C)nc(Nc2cc3c(c(C4=CCNC[C@H](O)C4)c2C)OCO3)n1] |
| 432 | (or) | 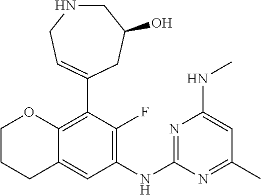 [SMILES: CNc1cc(C)nc(Nc2cc3c(c(C4=CCNC[C@@H](O)C4)c2F)OCCC3)n1] |
| 433(*) | (or) | 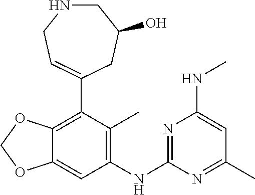 [SMILES: CNc1cc(C)nc(Nc2cc3c(c(C4=CCNC[C@@H](O)C4)c2C)OCO3)n1] |
| 434 | (or) | 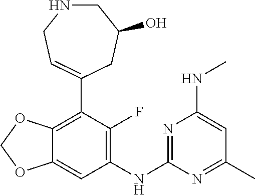 [SMILES: CNc1cc(C)nc(Nc2cc3c(c(C4=CCNC[C@@H](O)C4)c2F)OCO3)n1] |
| 435 | (or) | 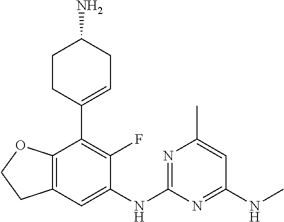 [SMILES: CNc1cc(C)nc(Nc2cc3c(c(C4=CC[C@@H](N)CC4)c2F)OCC3)n1] |
| 436 | (or) | 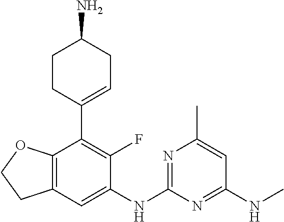 [SMILES: CNc1cc(C)nc(Nc2cc3c(c(C4=CC[C@H](N)CC4)c2F)OCC3)n1] |
| 437 | (and) | 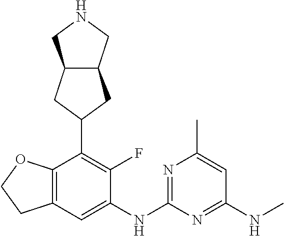 |

Note also that two reference compounds (Reference Compounds A and B) are referred to throughout but are not considered part of the disclosed embodiments. Reference Compounds A and B (synthesized as in Katayama, K., et al., Bioorganic and Med. Chem. Lett., 2020, 30, 127475) have the structures:

### Reference Compound A (compound 349)

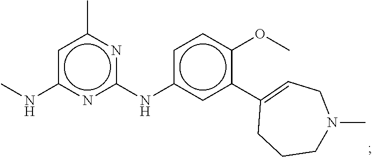

### Reference Compound B (Compound 177)

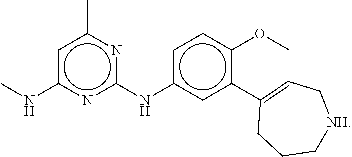

### Methods of Treatment

Provided herein, in certain embodiments, is a method of treating a disease or disorder that can be treated by modulation of EHMT1 or EHMT2, the method comprising administering to a patient in need thereof a compound described herein or a composition described herein.

In some embodiments, the disease or disorder is selected from the group consisting of cancer, sickle cell disease, and beta thalassemia.

In some embodiments, the disease or disorder is cancer (e.g., colorectal cancer).

In some embodiments the cancer is breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, or vulval cancer.

In some embodiments the cancer is ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, ophthalmological cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, or Wilm's tumor.

In some embodiments the cancer is a lymphoma. In some embodiments, the lymphoma is Hodgkin's lymphoma or non-Hodgkin's lymphoma. In some embodiments, the non-Hodgkin's lymphoma is selected from the group consisting of B-cell lymphomas (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenstrom's macroglobulinemia, hairy cell leukemia, and primary central nervous system (CNS) lymphoma) and T-cell lymphomas (e.g., precursor T-lymphoblastic lymphoma/leukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

In some embodiments, the cancer is selected from the group consisting of a melanoma, bladder cancer, colorectal cancer, head and neck cancer, esophageal cancer, liver cancer, lung cancer, pancreas cancer, and stomach cancer.

In some embodiments, the method further comprises use of at least one additional therapeutic agent. In some embodiments, the at least one additional therapeutic agent is chemotherapy or radiation.

In another aspect, provided is a use of a compound of the disclosure in the manufacture of a medicament for the treatment of cancer.

Cancers: Cancer cells grow quickly and in low oxygen environments by activating different elements of the cellular stress response. Without wishing to be bound by a theory, compounds of Formula (I) may also be used for treatment of cancer, as a greater understanding of the role of EHMT in cancer has recently begun to emerge. Additionally, EHMT modulators can be combined with one or more cancer therapies, such as chemotherapy and radiation therapy.

A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and the like.

Other exemplary cancers include, but are not limited to, ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, ophthalmological cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, Wilm's tumor, and the like.

Exemplary lymphomas include Hodgkin's lymphoma and non-Hodgkin's lymphoma. Further exemplification of non-Hodgkin's lymphoma include, but are not limited to, B-cell lymphomas (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenstrom's macroglobulinemia, hairy cell leukemia, and primary central nervous system (CNS) lymphoma) and T-cell lymphomas (e.g., precursor T-lymphoblastic lymphoma/leukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the terms “compounds” and “agent” are used interchangeably to refer to the inhibitors/antagonists/agonists of the invention. In certain embodiments, the compounds are small organic or inorganic molecules, e.g., with molecular weights less than 7500 amu, preferably less than 5000 amu, and even more preferably less than 2000, 1500, 1000, 750, 600, or 500 amu. In certain embodiments, one class of small organic or inorganic molecules are non-peptidyl, e.g., containing 2, 1, or no peptide and/or saccharide linkages.

Unless otherwise indicated, all numbers expressing quantities of ingredients or reaction conditions used herein should be understood as modified in all instances by the term “about.” The term “about” when used in connection with percentages may mean+1%.

The singular terms “a,” “an,” and “the” refer to one or to more than one, unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, intrathecal, and topical (including buccal and sublingual) administration.

The terms “decrease”, “reduced”, “reduction”, “decrease” or “inhibit” are all used herein generally to mean a decrease by a statistically significant amount. In some embodiments, the terms “reduced”, “reduction”, “decrease” or “inhibit” mean a decrease by at least 0.1% as compared to a reference level, for example a decrease by at least about 1%, or at least about 5%, or at least about 10%, or at least about 15%, or at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 1-100%, e.g., 10-100% as compared to a reference level.

The terms “increased”, “increase”, “enhance” or “activate” are all used herein to generally mean an increase by a statically significant amount. In some embodiments, the terms “increased”, “increase”, “enhance” or “activate” mean an increase by at least 0.1% as compared to a reference level, for example a decrease by at least about 1%, or at least about 5%, or at least about 10%, or at least about 15%, or at least about 20%, or at least about 30%, or at least about 40%, or at least about 50%, or at least about 60%, or at least about 70%, or at least about 80%, or at least about 90% or up to and including a 100% increase (e.g. absent level as compared to a reference sample), or any increase between 1-100%, e.g., 10-100% as compared to a reference level.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, at least one symptom of a disease or disorder is alleviated by at least about 1%, or at least about 5%, or at least about 10%, or at least about 15%, or at least about 20%, or at least about 30%, or at least about 40%, or at least about 50%.

As used herein, an amount of a compound or combination effective to treat a disorder (e.g., a disorder as described herein), “therapeutically effective amount” or “effective amount” refers to an amount of the compound or combination which is effective, upon single or multiple dose administration(s) to a subject, in treating a subject, or in curing, alleviating, relieving or improving a subject with a disorder (e.g., a disorder as described herein) beyond that expected in the absence of such treatment. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

The term “nucleic acid” as used herein refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

As used herein, the term “modulator of EHMT” refers to compounds and compositions of Formula (I) that modulate the activity of EHMT, e.g., EHMT1 and EHMT2.

### Selected Chemical Definitions

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, propyl, butyl, pentyl and hexyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

If a compound of the present invention is depicted in the form of a chemical name and as a formula, in case of any discrepancy, the formula shall prevail.

The symbol [symbol] , whether utilized as a bond or displayed perpendicular to a bond indicates the point at which the displayed moiety is attached to the remainder of the molecule, solid support, etc.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-10 alkyl (e.g., —CH₃). In certain embodiments, the alkyl group is substituted C₁-6 alkyl.

The term “alkylene” refers to a diradical of an alkyl group. An exemplary alkylene group is —CH₂CH₂—.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₀ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-4 alkenyl groups as well as pentenyl (C), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”).

In some embodiments, an alkynyl group has 2 to 6 carbon atoms (“C₂-C₆ alkynyl”). In some embodiments, an alkynyl group has 2 to 5 carbon atoms (“C₂-C₈ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —NHCH₂—, —C(O)NH—, —C(O)N(CH₃), —C(O)N(CH₂CH₃)—, —C(O)N(CH₂CF₃)—, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. One type of heteroalkyl group is an “alkoxyl” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘₘ—R^{aaa}, where mm is an integer (e.g., 1, 2, 3, 4, 5, 6, 7, 8, 9, 10, or 11) and R^{aaa} may be halogen, haloalkyl, nitrile, —NH₂, —NO₂, —SO₂, Si(CH₃)₃, cycloalkyl, heterocyclyl, aryl, or heteroaryl. are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The terms “hydroxyalkyl” refers to an alkyl group, as defined above, wherein one or more (e.g., one, two or three) of the hydrogen atoms are independently replaced with a hydroxyl group (—OH). Exemplary hydroxyalkyl groups include —CH₂OH, CH₂CH₂OH, C(CH₃)₂OH and the like As used herein, “aryl” refers to a radical of a monocyclic or polycyclic (e.g., bicyclic or tricyclic) 4n+2 aromatic ring system (e.g., having 6, 10, or 14 R electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 R electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives. “heteroaryl” also includes ring systems wherein the heteroaryl ring, as defined above, is fused with one or more heterocycloalkyl groups wherein the point of attachment is on the heteroaryl ring, and in such instances, the number of carbons continue to designate the number of carbons in the heteroaryl ring system. Exemplary ring systems of this type include 7,8-dihydro-5H-pyrano[4,3-b]pyridine and 1,4,6,7-tetahydropyrano[4,3-b]pyrrole.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl. “Heterocyclyl,” “heterocycle” or “heterocycloalkyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl” or “C₃-C₁₀ heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl or aryl or heteroaryl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Alternatively, a heterocyclyl group containing 3-10 non-hydrogen ring atoms i.e., carbon, nitrogen, oxygen, sulfur, boron, phosphorus, and silicon, may be described as a “C₃-C₁₀ heterocyclyl”; a heterocyclyl group containing 3-7 non-hydrogen ring atoms i.e., carbon, nitrogen, oxygen, sulfur, boron, phosphorus, and silicon, may be described as a “C₃-C₇ heterocyclyl. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl (i.e., unsubstituted C₃-C₁₀ heterocyclyl). In certain embodiments, the heterocyclyl group is substituted 3-10 membered heterocyclyl (i.e., substituted C₃-C₁₀ heterocyclyl).

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, azacyclohexenyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, azacycloheptenyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “haloalkyl” can include alkyl structures that are substituted with one or more halo groups or with combinations thereof. For example, the terms “fluoroalkyl” includes haloalkyl groups in which the halo is fluorine (e.g., —C₁-C₆ alkyl-CF₃, —C₁-C₆ alkyl-C₂F). Non-limiting examples of haloalkyl include trifluoroethyl, trifluoropropyl, trifluoromethyl, fluoromethyl, difluoromethyl, and fluroisopropyl.

As used herein, “hydroxy” refers to the radical —OH.

As used herein, “nitro” refers to —NO₂.

As used herein, “oxo” refers to ═O, in which both bonds from the oxygen are connected to the same atom. For example, a carbon atom substituted with oxo forms a carbonyl group —C═O.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

Many of the terms given above may be used repeatedly in the definition of a formula or group and in each case have one of the meanings given above, independently of one another.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. An optionally substituted moiety can be substituted with groups that, together with the atoms to which they are attached, form a ring (e.g., a 3-10 member cycloalkyl or heterocyclyl). Unless otherwise specified, substituents on one “optionally substituted” moiety cannot be taken together with substituents on a second, distinct “optionally substituted” moiety to form a ring. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable substituents for an optionally substituted alkyl, alkylene, heteroalkyl, heteroalkylene, carbocyclyl, heterocyclyl, aryl group and heteroaryl group include halogen, ═O, —CN, —OR^{cc}, —NR^{dd}R^{ee}, —S(O)^{kk}R^{cc}, —NR^{cc}S(O)₂R^{cc}, —S(O)₂NR^{dd}R^{ee}, —C(═O)OR^{cc}, —OC(═O)OR^{cc}, —OC(═O)R^{cc}, —OC(═S)OR^{cc}, —C(═S)OR^{cc}, —O(C═S) R^{cc}, —C(═O)NR^{dd}R^{ee}, —NR^{cc}C(═O)R^{cc}, —C(═S)NR^{dd}R^{ee}, —NR^{cc}C(═S)R^{cc}, —NR^{cc}(C═O)OR^{cc}, —O(C═O)NR^{dd}R^{ee}, —NR^{cc}(C═S)OR^{cc}, —O(C═S)NR^{dd}R^{ee}, —NR^{cc}(C═O)NR^{dd}R^{ee}, —NR^{cc}(C═S)NR^{dd}R^{ee}, —C(═S)R^{cc}, —C(═O)R^{cc}, C₁-C₆ alkyl, C₁-C₆ haloalkyl, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, wherein each of said alkyl, alkylene, heteroalkyl, heteroalkylene, carbocyclyl, heterocyclyl, aryl and heteroaryl are optionally substituted with one or more of halogen, ORc, —NO₂, —CN, —NR^{cc}C(═O)W, —NR^{dd}R^{ee}, —S(O)ₖR^{cc}, —C(═O)OR^{cc}, —C(═O)NR^{dd}R^{ee}, —C(═O)R^{cc}, C₁-C₆ alkyl, C₁-C₆ haloalkyl, or C₁-C₆ heteroalkyl, and wherein R^{cc} is hydrogen, hydroxy, C₁-C₆ alkyl, C₁-C₆ heteroalkyl, carbocyclyl, (C₁-C₆-alkylene)-carbocyclyl, (C₁-C₆-heteroalkylene)-carbocyclyl, heterocyclyl, (C₁-C₆-alkylene)-heterocyclyl, (C₁-C₆-heteroalkylene)-heterocyclyl, aryl, (C₁-C₆-alkylene)-aryl, (C₁-C₆-heteroalkylene)-aryl, heteroaryl, (C₁-C₆-alkylene)-heteroaryl, or (C₁-C₆-heteroalkylene)-heteroaryl, each of which is optionally substituted with one or more of halogen, hydroxy, C₁-C₆ alkyl, C₁-C₆ haloalkyl, C₁-C₆ heteroalkyl, carbocyclyl, heterocyclyl, aryl, or heteroaryl; R^{dd} and R^{ee} are each independently selected from hydrogen, C₁-C₆ alkyl, or C₁-C₆ heteroalkyl; and k is 0, 1 or 2. The invention is not intended to be limited in any manner by the above exemplary listing of substituents.

Contemplated equivalents of the compounds described above include compounds which otherwise correspond thereto, and which have the same general properties thereof (e.g., the ability to modulate EHMT1 or EHMT2), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### Pharmaceutical Compositions and Routes of Administration

Pharmaceutical compositions containing compounds described herein such as a compound of Formula (I) or (II) or pharmaceutically acceptable salt thereof can be used to treat or ameliorate a disorder described herein, for example, a neurodegenerative disease, a cancer, an ophthalmological disease (e.g., a retinal disease), or a viral infection.

The amount and concentration of compounds of Formula (I) or (II) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound of the present invention to be administered alone, it is preferable to administer the compound as a pharmaceutical formulation (composition), where the compound is combined with one or more pharmaceutically acceptable diluents, excipients or carriers. The compounds according to the invention may be formulated for administration in any convenient way for use in human or veterinary medicine. In certain embodiments, the compound included in the pharmaceutical preparation may be active itself, or may be a prodrug, e.g., capable of being converted to an active compound in a physiological setting. Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) intrathecally. Additionally, compounds can be implanted into a patient or injected using a drug delivery system. See, for example, Urquhart, et al., (1994) Ann Rev Pharmacol Toxicol 24:199-236; Lewis, ed. “Controlled Release of Pesticides and Pharmaceuticals” (Plenum Press, New York, 1981); U.S. Pat. No. 3,773,919; and U.S. Patent No. 35 3,270,960.

The phrase “therapeutically effective amount” as used herein means that amount of a compound, material, or composition comprising a compound of the present invention, which is effective for producing some desired therapeutic effect, e.g., by modulating EHMT1 or EHMT2, in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

The term “pharmaceutically acceptable salt” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the heart, lung, bladder, urethra, ureter, rectum, or intestine. Furthermore, compositions can be formulated for delivery via a dialysis port.

Ophthalmic formulations, eye ointments, powders, solutions and the like, are also contemplated as being within the scope of this invention.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with neurodegenerative disease or disorder, cancer, or viral infections.

In addition, the methods described herein can be used to treat domesticated animals and/or pets. A subject can be male or female. A subject can be one who has been previously diagnosed with or identified as suffering from or having a neurodegenerative disease or disorder, a disease or disorder associated with cancer, a disease or disorder associated with viral infection, or one or more complications related to such diseases or disorders but need not have already undergone treatment.

### Dosages

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

The compound and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other agent. When the inhibitor and the pharmaceutically active agent are administered in different pharmaceutical compositions, routes of administration can be different.

The amount of compound that can be combined with a carrier material to produce a single dosage form will generally be that amount of the inhibitor that produces a therapeutic effect. Generally out of one hundred percent, this amount will range from about 0.1% to 99% of inhibitor, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment.

The present invention contemplates formulation of the subject compounds in any of the aforementioned pharmaceutical compositions and preparations. Furthermore, the present invention contemplates administration via any of the foregoing routes of administration. One of skill in the art can select the appropriate formulation and route of administration based on the condition being treated and the overall health, age, and size of the patient being treated.

### SELECTED EMBODIMENTS

Embodiment 1. A compound of formula (I) or a pharmaceutically acceptable salt thereof

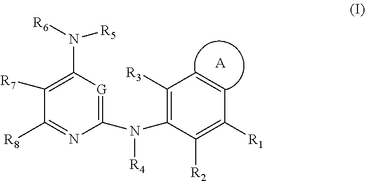

wherein,
A is an optionally substituted 5- or 6-membered oxygen-containing heterocyclic ring;
G is N or CR⁷;
R¹ is —O—C₁-C₆ alkylene-E, —NR¹²-C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

Embodiment 2. A compound of formula (I) or a pharmaceutically acceptable salt thereof

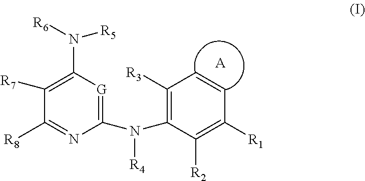

wherein,
A is an optionally substituted 5- or 6-membered oxygen-containing heterocyclic ring;
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

Embodiment 3. The compound of embodiment 1 or 2, or a pharmaceutically acceptable salt thereof, wherein A is an optionally substituted 6-membered oxygen-containing heterocyclic ring.

Embodiment 4. The compound of embodiment 1 or 2, or a pharmaceutically acceptable salt thereof, wherein A is an optionally substituted 5-membered oxygen-containing heterocyclic ring.

Embodiment 5. A compound of formula (II), or a pharmaceutically acceptable salt thereof

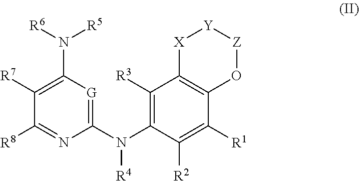

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

Embodiment 6. A compound of formula (II), or a pharmaceutically acceptable salt thereof

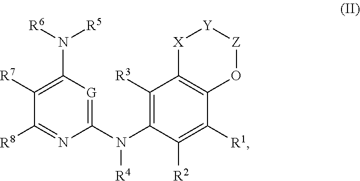

wherein X is C(R¹¹)₂, O, S(O)_{w}, or NR¹²;
Y is a bond, C(R¹³)₂, or C(R¹³)₂—C(R¹³)₂; and
Z is CR⁹R¹⁰;
or X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

Embodiment 7. A compound of formula (II)

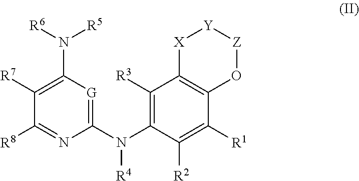

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH; and
w is 0, 1, or 2.

Embodiment 8. A compound of formula (II), or a pharmaceutically acceptable salt thereof

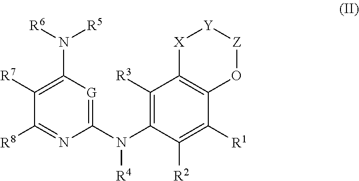

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl and OH, wherein R^{E} is optionally substituted by one or more deuteriums; and
w is 0, 1, or 2.

Embodiment 9. A compound of formula (II), or a pharmaceutically acceptable salt thereof

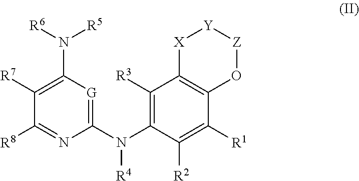

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
or R⁹ and R¹⁰ can be taken together with the carbon to which they are attached to form CO;
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂ and OH, wherein R^{E} is optionally substituted by one or more deuteriums; and
w is 0, 1, or 2.

Embodiment 10. A compound of formula (II)

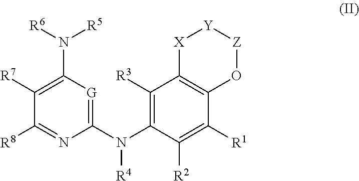

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, and OH.

Embodiment 11. A compound of formula (II), or a pharmaceutically acceptable salt thereof

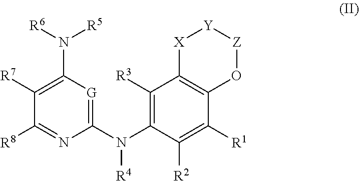

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen, each alkyl or heteroalkyl optionally substituted with 1-5 deuteriums;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, and OH, wherein R^{E} is optionally substituted by one or more deuteriums.

Embodiment 12. A compound of formula (II), or a pharmaceutically acceptable salt thereof

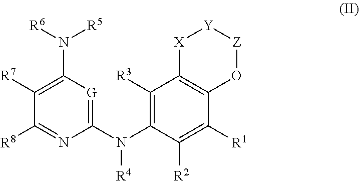

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, 3-heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen, deuterium and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, each alkyl or heteroalkyl optionally substituted with 1-5 deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂, and OH, wherein R^{E} is optionally substituted by one or more deuteriums.

Embodiment 13. The compound of any one of embodiments 1-12, or a pharmaceutically acceptable salt thereof, wherein G is CR⁷.

Embodiment 14. The compound of any one of embodiments 1-12, or a pharmaceutically acceptable salt thereof, wherein G is CH.

Embodiment 15. The compound of any one of embodiments 1-12, or a pharmaceutically acceptable salt thereof, wherein G is N.

Embodiment 16. A compound of formula (III), or a pharmaceutically acceptable salt thereof

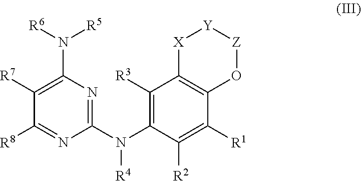

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

Embodiment 17. A compound of formula (III), or a pharmaceutically acceptable salt thereof

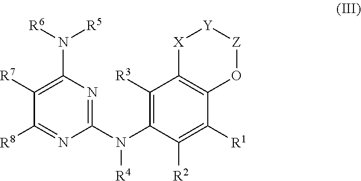

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂—C₁-C₆ alkyl; SO_{w}—C₁-C₆ alkyl; CO₂—C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

Embodiment 18. A compound of formula (III)

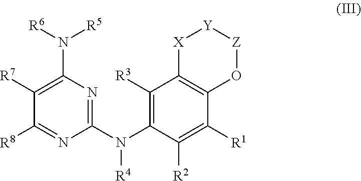

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH;
w is 0, 1, or 2.

Embodiment 19. A compound of formula (III), or a pharmaceutically acceptable salt thereof

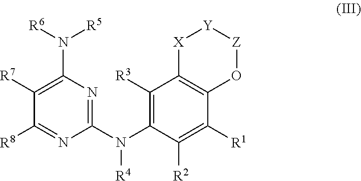

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, and OH, wherein R^{E} is optionally substituted by one or more deuteriums; and
w is 0, 1, or 2.

Embodiment 20. A compound of formula (III), or a pharmaceutically acceptable salt thereof

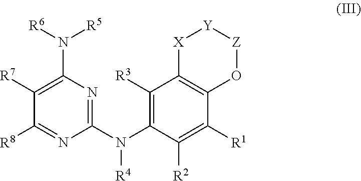

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted with 1-4 R^{E};
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E};
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E};
each R⁹ and R¹⁰ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E};
or R⁹ and R¹⁰ can be taken together with the carbon to which they are attached to form CO;
each R¹¹ and R¹³ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, hydroxy, cyano, CO—R^{C}, NR^{D}₂, or halogen, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹¹ or two R¹³ can be taken together with the carbon to which they are attached to form CO or a spirofused C₃-C₇ cycloalkyl;
each R¹² is H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 R^{E};
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH;
each R^{D} is independently H, C₁-C₆ alkyl, CO—C₁-C₆ alkyl; CO₂—C₁-C₆ alkyl; SO_{w}—C₁-C₆ alkyl; C₁-C₆ heteroalkyl, C₃-C₇ cycloalkyl, or heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 substituents independently selected from halogen, C₁-C₆ alkyl, C₁-C₆ haloalkyl, C₁-C₆ alkoxy, and OH; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted with 1-4 substituents independently selected from halogen, C₁-C₆ alkyl, C₁-C₆ haloalkyl, C₁-C₆ alkoxy, and OH;
each R^{E} is independently selected from halogen, C₁-C₆ alkyl, C₁-C₆ haloalkyl, C₁-C₆ alkoxy, C₃-C₇ cycloalkyl, heterocyclyl, C₁-C₆ heteroalkyl, C₁-C₆ hydroxyalkyl, NH₂, and OH, wherein R^{E} is optionally substituted by one or more deuteriums;
w is 0, 1, or 2.

Embodiment 21. A compound of formula (III)

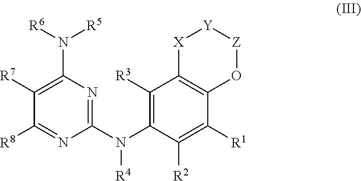

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; or
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, and OH; and
w is 0, 1, or 2.

Embodiment 22. A compound of formula (III), or a pharmaceutically acceptable salt thereof

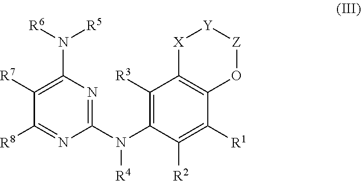

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
R′ is H or C₁-C₆ alkyl;
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; or
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, and OH, wherein R^{E} is optionally substituted by one or more deuteriums; and
w is 0, 1, or 2.

Embodiment 23. A compound of formula (III), or a pharmaceutically acceptable salt thereof

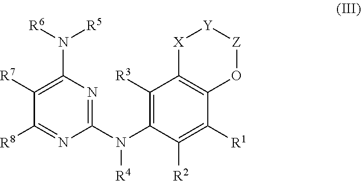

wherein
X is C(R¹¹)₂, O, S(O)_{w}, or NR¹²;
Y is a bond, C(R¹³)₂, or C(R¹³)₂—C(R¹³)₂; and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E, wherein each methylene group in C₁-C₆ alkylene is individually optionally replaced by O or NR′;
E is C₃-C₁₀ cycloalkyl or heterocycloalkyl, each of which is optionally substituted with 1-4 R^{E};
R′ is H or C₁-C₆ alkyl;
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, C₃-C₇ cycloalkyl, heterocyclyl, C₁-C₆ hydroxyalkyl, NH₂, and OH, wherein R^{E} is optionally substituted by one or more deuteriums; and
w is 0, 1, or 2.

Embodiment 24. A compound of formula (III)

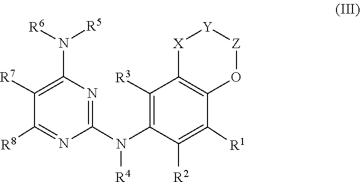

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-4 substituents independently selected from halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, and OH.

Embodiment 25. A compound of formula (III), or a pharmaceutically acceptable salt thereof

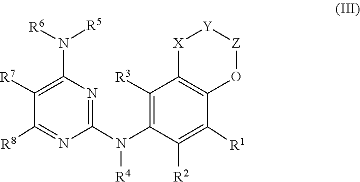

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl; and
each R^{E} is independently selected from halogen, C₁-C₆ alkyl, C₁-C₆ haloalkyl, C₁-C₆ heteroalkyl, C₁-C₆ alkoxy, and OH, wherein R^{E} is optionally substituted by one or more deuteriums.

Embodiment 26. A compound of formula (III), or a pharmaceutically acceptable salt thereof

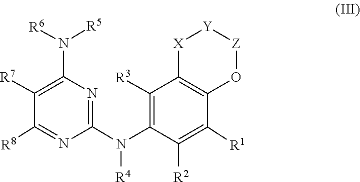

wherein
X is C(R¹¹)₂, O, or NR¹²;
Y is a bond or C(R¹³)₂, and
Z is CR⁹R¹⁰; or
X—Y is C(R¹¹)═C(R¹³) and Z is CR⁹R¹⁰; or
X—Y—Z is C(R¹¹)═C(R¹³);
R¹ is —O—C₁-C₆ alkylene-E, —NR¹²—C₁-C₆ alkylene-E, or E;
E is C₃-C₁₀ cycloalkyl or heterocycloalkyl, each of which is optionally substituted with 1-4 R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂ and OH, wherein R^{E} is optionally substituted by one or more deuteriums.

Embodiment 27. The compound of any one of embodiments 5-26, or a pharmaceutically acceptable salt thereof, wherein X is C(R¹¹)₂ or O.

Embodiment 28. The compound of any one of embodiments 5-26, or a pharmaceutically acceptable salt thereof, wherein X is CHR¹, CH₂ or O.

Embodiment 29. The compound of any one of embodiments 5-26, or a pharmaceutically acceptable salt thereof, wherein X is CH(CH₃), CH₂ or O.

Embodiment 30. The compound of any one of embodiments 5-26, or a pharmaceutically acceptable salt thereof, wherein X is CH(CH₃), or O.

Embodiment 31. The compound of any one of embodiments 5-26, or a pharmaceutically acceptable salt thereof, wherein X is CH₂ or O.

Embodiment 32. The compound of any one of embodiments 5-26, or a pharmaceutically acceptable salt thereof, wherein X is C(R¹¹)₂.

Embodiment 33. The compound of any one of embodiments 5-26, or a pharmaceutically acceptable salt thereof, wherein X is CH₂.

Embodiment 34. The compound of any one of embodiments 5-26, or a pharmaceutically acceptable salt thereof, wherein X is CH(CH₃).

Embodiment 35. The compound of any one of embodiments 5-26, or a pharmaceutically acceptable salt thereof, wherein X is O.

Embodiment 36. The compound of any one of embodiments 5-26, or a pharmaceutically acceptable salt thereof, wherein X is NR¹².

Embodiment 37. The compound of any one of embodiments 5-36, or a pharmaceutically acceptable salt thereof, wherein Y is C(R¹³)₂.

Embodiment 38. The compound of any one of embodiments 5-36, or a pharmaceutically acceptable salt thereof, wherein Y is a bond or CH₂.

Embodiment 39. The compound of any one of embodiments 5-36, or a pharmaceutically acceptable salt thereof, wherein Y is CH₂.

Embodiment 40. The compound of any one of embodiments 5-36, or a pharmaceutically acceptable salt thereof, wherein Y is a bond.

Embodiment 41. The compound of any one of embodiments 5-40, or a pharmaceutically acceptable salt thereof, wherein X—Y is CH═CH.

Embodiment 42. The compound of any one of embodiments 5-26, or a pharmaceutically acceptable salt thereof, wherein X—Y—Z is CH═CH.

Embodiment 43. The compound of any one of embodiments 5-26, or a pharmaceutically acceptable salt thereof, wherein X is C(R¹¹)₂ and Y is C(R¹³)₂.

Embodiment 44. The compound of any one of embodiments 5-26, or a pharmaceutically acceptable salt thereof, wherein X is CH₂ and Y is CH₂.

Embodiment 45. The compound of any one of embodiments 5-26, or a pharmaceutically acceptable salt thereof, wherein X is C(R¹¹)₂ and Y is a bond.

Embodiment 46. The compound of any one of embodiments 5-26, or a pharmaceutically acceptable salt thereof, wherein X is CH₂ and Y is a bond.

Embodiment 47. The compound of any one of embodiments 5-26, or a pharmaceutically acceptable salt thereof, wherein X is O and Y is C(R¹³)₂.

Embodiment 48. The compound of any one of embodiments 5-26, or a pharmaceutically acceptable salt thereof, wherein X is O and Y is CH₂.

Embodiment 49. The compound of any one of embodiments 5-26, or a pharmaceutically acceptable salt thereof, wherein X is O and Y is a bond.

Embodiment 50. The compound of any one of embodiments 1-49, or a pharmaceutically acceptable salt thereof, wherein R¹ is —NR¹²—C₁-C₆ alkylene-E.

Embodiment 51. The compound of any one of embodiments 1-49, or a pharmaceutically acceptable salt thereof, wherein R¹ is selected from E and —O—C₁-C₆ alkylene-E.

Embodiment 52. The compound of any one of embodiments 1-49, or a pharmaceutically acceptable salt thereof, wherein R¹ is —O—C₁-C₆ alkylene-E.

Embodiment 53. The compound of any one of embodiments 1-49, or a pharmaceutically acceptable salt thereof, wherein R¹ is selected from —O—(CH₂)₂-E and —O—(CH₂)₃-E.

Embodiment 54. The compound of any one of embodiments 1-49, or a pharmaceutically acceptable salt thereof, wherein R¹ is E.

Embodiment 55. The compound of any one of embodiments 1-54, or a pharmaceutically acceptable salt thereof, wherein E is C₃-C₁₀ cycloalkyl optionally substituted with 1-4 R^{E}.

Embodiment 56. The compound of any one of embodiments 1-54, or a pharmaceutically acceptable salt thereof, wherein E is cyclohexyl or cyclohexenyl optionally substituted with 1-4 R^{E}.

Embodiment 57. The compound of any one of embodiments 1-54, or a pharmaceutically acceptable salt thereof, wherein E is C₃-C₁₀ heterocycloalkyl optionally substituted with 1-4 R^{E}.

Embodiment 58. The compound of embodiment 57, or a pharmaceutically acceptable salt thereof, wherein the heterocycloalkyl has 3-10 ring atoms including 1-3 ring heteroatoms selected from N, O, and S.

Embodiment 59. The compound of embodiment 57, or a pharmaceutically acceptable salt thereof, wherein the heterocycloalkyl has 5-8 ring atoms including 1-3 ring heteroatoms selected from N, O, and S.

Embodiment 60. The compound of embodiment 57, or a pharmaceutically acceptable salt thereof, wherein the heterocycloalkyl has 5-8 ring atoms including 1 or 2 nitrogen heteroatoms.

Embodiment 61. The compound of embodiment 57, or a pharmaceutically acceptable salt thereof, wherein the heterocycloalkyl has 5-8 ring atoms including 1 nitrogen heteroatom.

Embodiment 62. The compound of embodiment 57, or a pharmaceutically acceptable salt thereof, wherein the heterocycloalkyl has 6-8 ring atoms including 1-3 ring heteroatoms selected from N, O, and S.

Embodiment 63. The compound of embodiment 57, or a pharmaceutically acceptable salt thereof, wherein the heterocycloalkyl has 6-8 ring atoms including 1 or 2 nitrogen heteroatoms.

Embodiment 64. The compound of embodiment 57, or a pharmaceutically acceptable salt thereof, wherein the heterocycloalkyl has 6-8 ring atoms including 1 nitrogen heteroatom.

Embodiment 65. The compound of any one of embodiments 1-54, or a pharmaceutically acceptable salt thereof, wherein E is selected from pyrrolidinyl, piperidinyl, piperazinyl, tetrahydropyridinyl, azepanyl, diazepanyl, tetrahydro-1H-azepinyl, 2,6-diazaspiro[3.5]nonanyl, 2,6-diazaspiro[3.4]octanyl, hexahydrocyclopenta[c]pyrrolyl, 1,8-diazaspiro[4.5]decanyl, 1,7-diazaspiro[4.4]nonanyl, 1,7-diazaspiro[4.5]decanyl, 2,7-diazaspiro[4.4]nonanyl, 2,8-diazaspiro[4.5]decanyl, 2,7-diazaspiro[4.5]decanyl, cyclohexenyl, octahydrocyclopenta[c]pyrrolyl and octahydropyrrolo[3,4-c]pyrrolyl, each optionally substituted with 1-4 R^{E}.

Embodiment 66. The compound of any one of embodiments 1-54, or a pharmaceutically acceptable salt thereof, wherein E is selected from pyrrolidinyl, piperidinyl, piperazinyl, tetrahydropyridinyl, azepanyl, diazepanyl, tetrahydro-1H-azepinyl, cyclohexenyl, hexahydrocyclopenta[c]pyrrolyl, octahydrocyclopenta[c]pyrrolyl and octahydropyrrolo[3,4-c]pyrrolyl, each optionally substituted with 1-4 R^{E}.

Embodiment 67. The compound of any one of embodiments 1-54, or a pharmaceutically acceptable salt thereof, wherein E is selected from pyrrolidinyl and tetrahydro-1H-azepinyl, each optionally substituted with 1-4 R^{E}.

Embodiment 68. The compound of any one of embodiments 1-54, or a pharmaceutically acceptable salt thereof, wherein E is pyrrolidinyl, optionally substituted with 1-4 R^{E}.

Embodiment 69. The compound of any one of embodiments 1-54, or a pharmaceutically acceptable salt thereof, wherein E is tetrahydro-1H-azepinyl optionally substituted with 1-4 R^{E}.

Embodiment 70. The compound of any one of embodiments 1-54, or a pharmaceutically acceptable salt thereof, wherein E is selected from pyrrolidine-1-yl, piperidin-1-yl, piperidin-4-yl, piperazin-1-yl, tetrahydropyridin-4-yl, azepan-4-yl, 1,4-diazepan-1-yl, 2,3,4,7-tetrahydro-1H-azepin-5-yl, cyclohexen-1-yl, 2,6-diazaspiro[3.5]nonan-2-yl, 2,6-diazaspiro[3.4]octan-2-yl, 1,8-diazaspiro[4.5]decan-8-yl, 1,7-diazaspiro[4.4]nonan-7-yl, 1,7-diazaspiro[4.5]decan-7-yl, 2,7-diazaspiro[4.4]nonan-2-yl, 2,8-diazaspiro[4.5]decan-2-yl, 2,7-diazaspiro[4.5]decan-2-yl, 2,3,6,7-tetrahydro-1H-azepin-4-yl, 1,2,3,3a,4,6a-hexahydrocyclopenta[c]pyrrol-5-yl, octahydrocyclopenta[c]pyrol-5-yl and octahydropyrrolo[3,4-c]pyrrol-2-yl, each optionally substituted with 1-4 R^{E}.

Embodiment 71. The compound of any one of embodiments 1-54, or a pharmaceutically acceptable salt thereof, wherein E is selected from pyrrolidine-1-yl, piperidin-1-yl, piperidin-4-yl, piperazin-1-yl, tetrahydropyridin-4-yl, azepan-4-yl, 1,4-diazepan-1-yl, 2,3,4,7-tetrahydro-1H-azepin-5-yl, cyclohexen-1-yl, 1,2,3,3a,4,6a-hexahydrocyclopenta[c]pyrrol-5-yl, octahydrocyclopenta[c]pyrol-5-yl and octahydropyrrolo[3,4-c]pyrrol-2-yl, each optionally substituted with 1-4 R^{E}.

Embodiment 72. The compound of any one of embodiments 1-54, or a pharmaceutically acceptable salt thereof, wherein E is selected from pyrrolidine-1-yl and 2,3,4,7-tetrahydro-1H-azepin-5-yl, each optionally substituted with 1-4 R^{E}.

Embodiment 73. The compound of any one of embodiments 1-54, or a pharmaceutically acceptable salt thereof, wherein E is pyrrolidine-1-yl, optionally substituted with 1-4 R^{E}.

Embodiment 74. The compound of any one of embodiments 1-54, or a pharmaceutically acceptable salt thereof, wherein E is 2,3,4,7-tetrahydro-1H-azepin-5-yl optionally substituted with 1-4 R^{E}.

Embodiment 75. The compound of any one of embodiments 1-54, or a pharmaceutically acceptable salt thereof, wherein E is

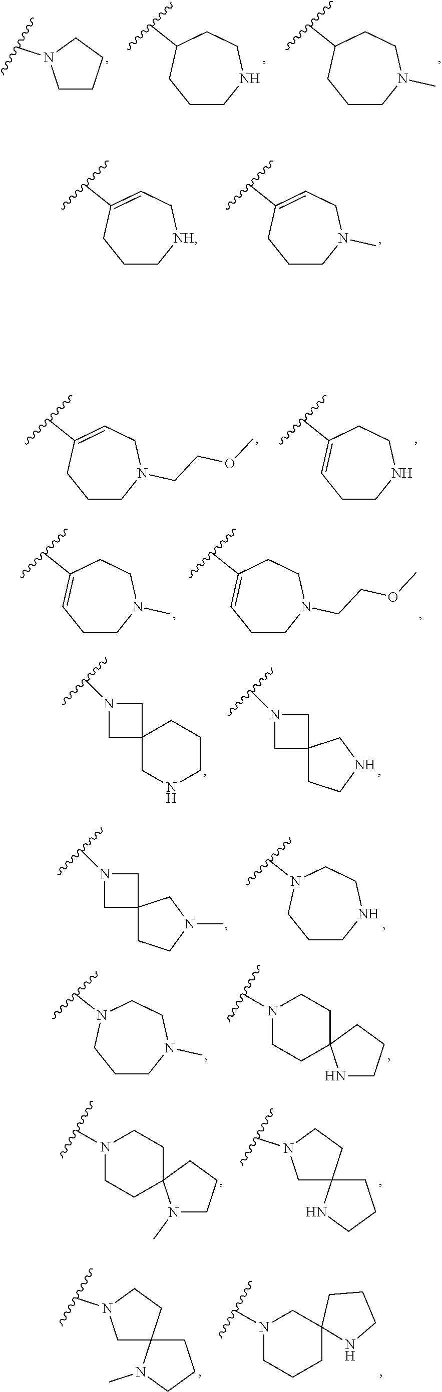

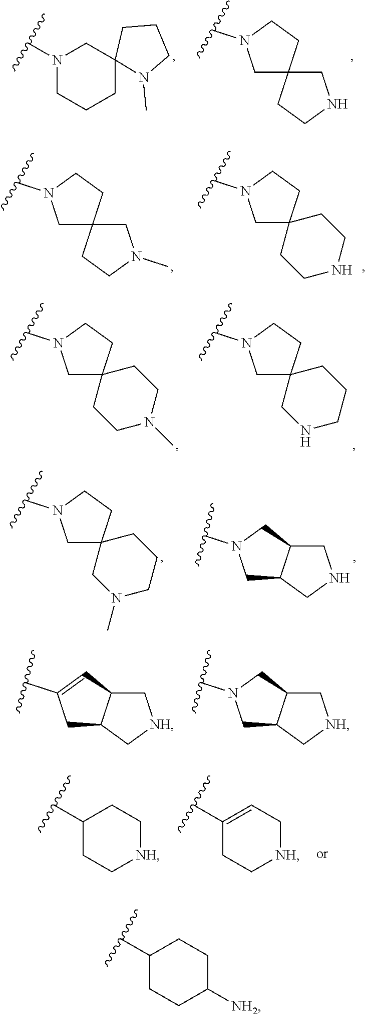

optionally substituted with 1-3 R^{E}.

Embodiment 76. The compound of any one of embodiments 1-54, or a pharmaceutically acceptable salt thereof, wherein E is

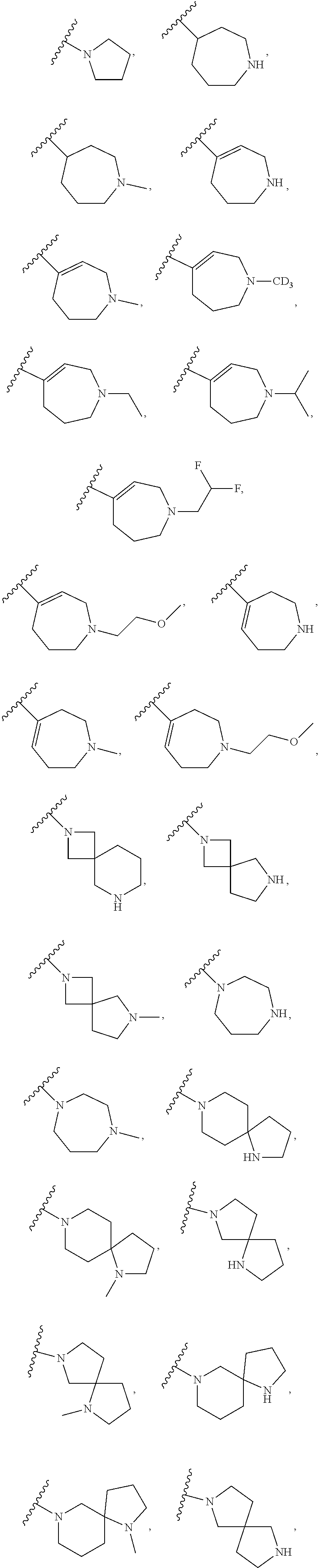

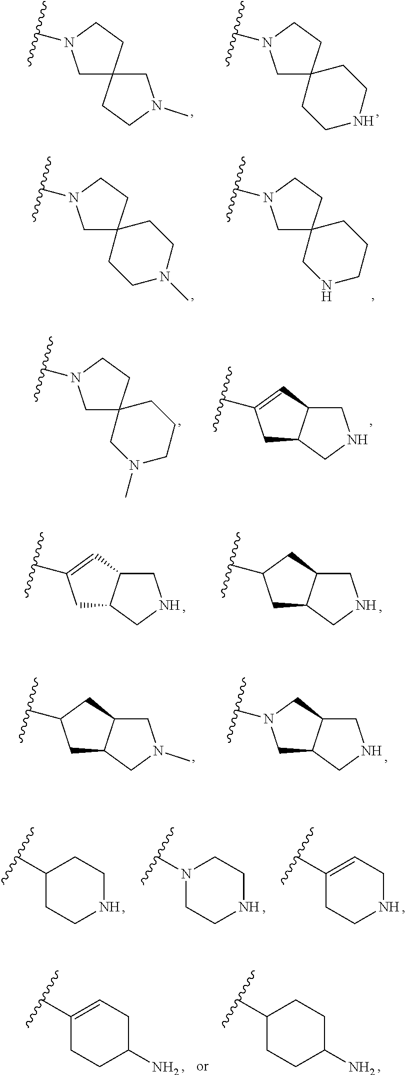

optionally substituted with 1-3 R^{E}.

Embodiment 77. The compound of any one of embodiments 1-54, or a pharmaceutically acceptable salt thereof, wherein E is

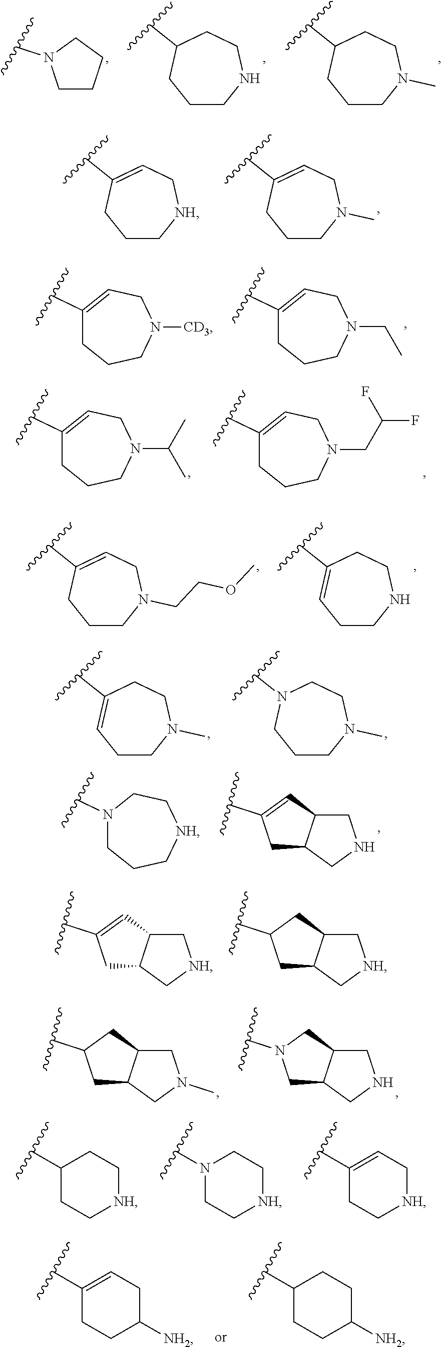

optionally substituted with 1-3 R^{E}.

Embodiment 78. The compound of any one of embodiments 1-54, or a pharmaceutically acceptable salt thereof, wherein E is

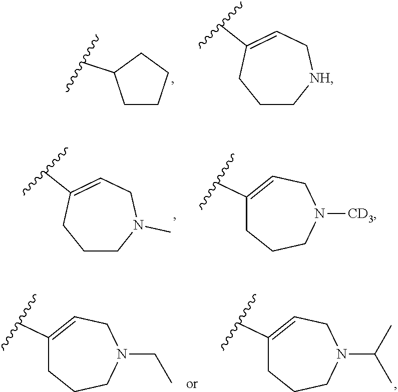

optionally substituted with 1-3 R^{E}.

Embodiment 79. The compound of any one of embodiments 1-54, or a pharmaceutically acceptable salt thereof, wherein E is

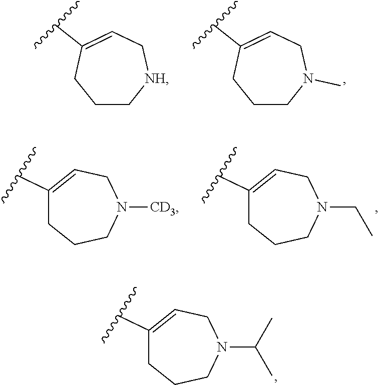

optionally substituted with 1-3 R^{E}.

Embodiment 80. The compound of any one of embodiments 1-54, or a pharmaceutically acceptable salt thereof, wherein E is

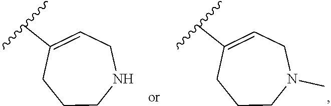

optionally substituted with 1-3 R^{E}.

Embodiment 81. The compound of any one of embodiments 1-54, or a pharmaceutically acceptable salt thereof, wherein E is

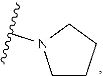 [SMILES: *N1CCCC1]

optionally substituted with 1-3 R^{E}.

Embodiment 82. The compound of any one of embodiments 1-54, or a pharmaceutically acceptable salt thereof, wherein E is

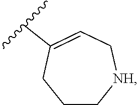 [SMILES: *C1=CCNCCC1]

optionally substituted with 1-3 R^{E}.

Embodiment 83. The compound of any one of embodiments 1-54, or a pharmaceutically acceptable salt thereof, wherein E is

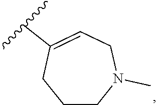 [SMILES: *C1=CCN(C)CCC1]

optionally substituted with 1-3 R^{E}.

Embodiment 84. The compound of any one of embodiments 1-54, or a pharmaceutically acceptable salt thereof, wherein E is

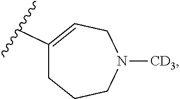 [SMILES: *C1=CCN(C([2H])([2H])[2H])CCC1]

optionally substituted with 1-3 R^{E}.

Embodiment 85. The compound of any one of embodiments 1-54, or a pharmaceutically acceptable salt thereof, wherein E is

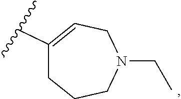 [SMILES: *C1=CCN(CC)CCC1]

optionally substituted with 1-3 R^{E}.

Embodiment 86. The compound of any one of embodiments 1-54, or a pharmaceutically acceptable salt thereof, wherein E is

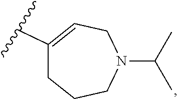 [SMILES: *C1=CCN(C(C)C)CCC1]

optionally substituted with 1-3 R^{E}.

Embodiment 87. The compound of any one of embodiments 1-54, or a pharmaceutically acceptable salt thereof, wherein E is

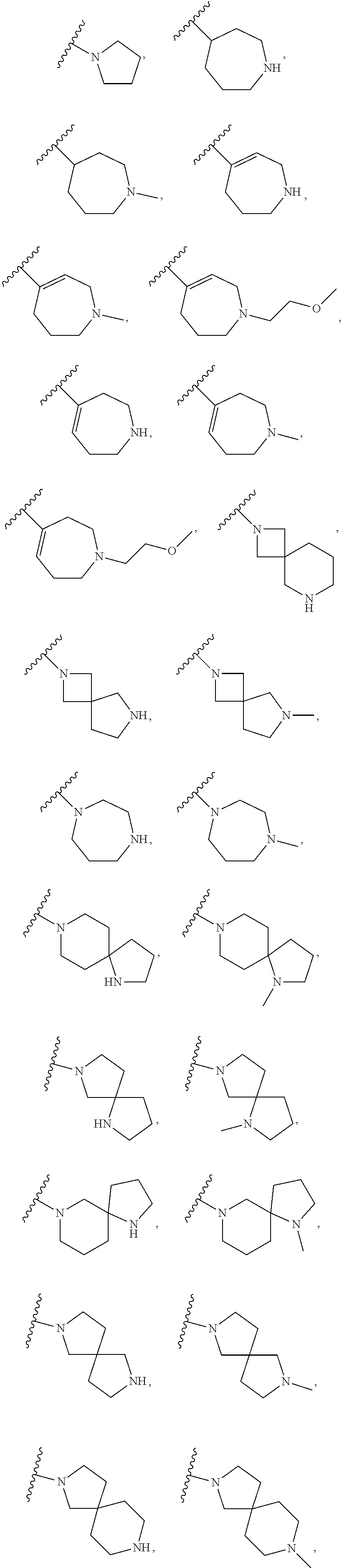

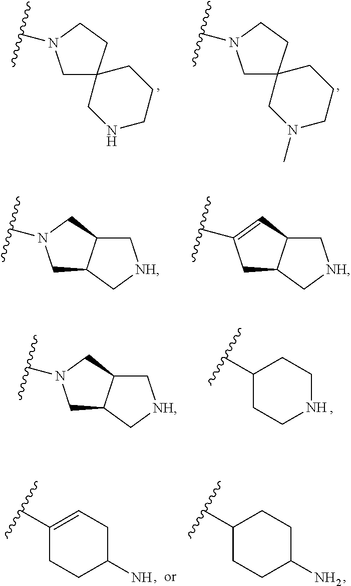

Embodiment 88. The compound of any one of embodiments 1-54, or a pharmaceutically acceptable salt thereof, wherein E is

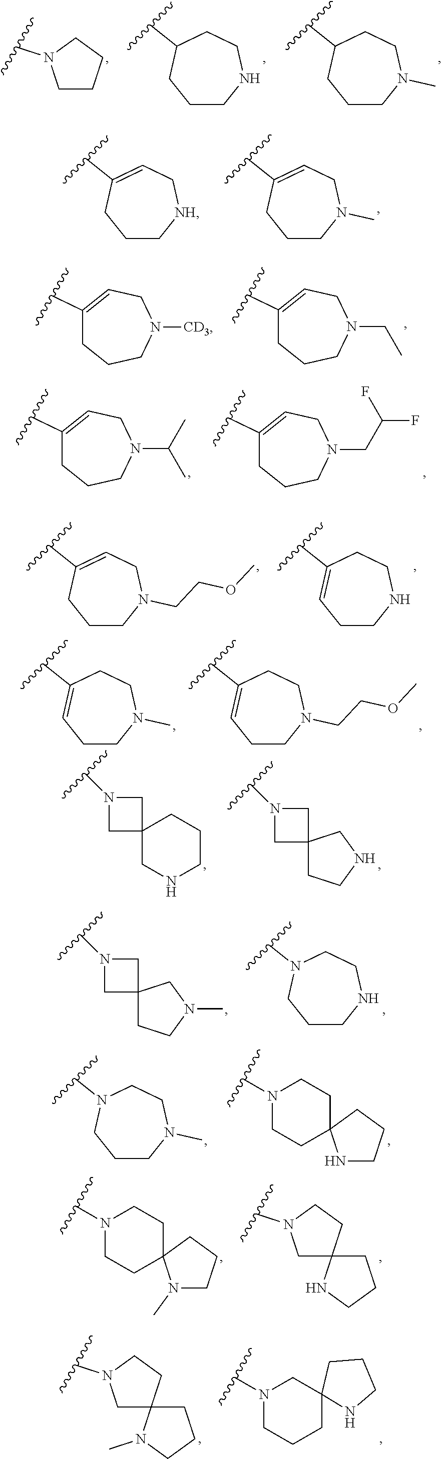

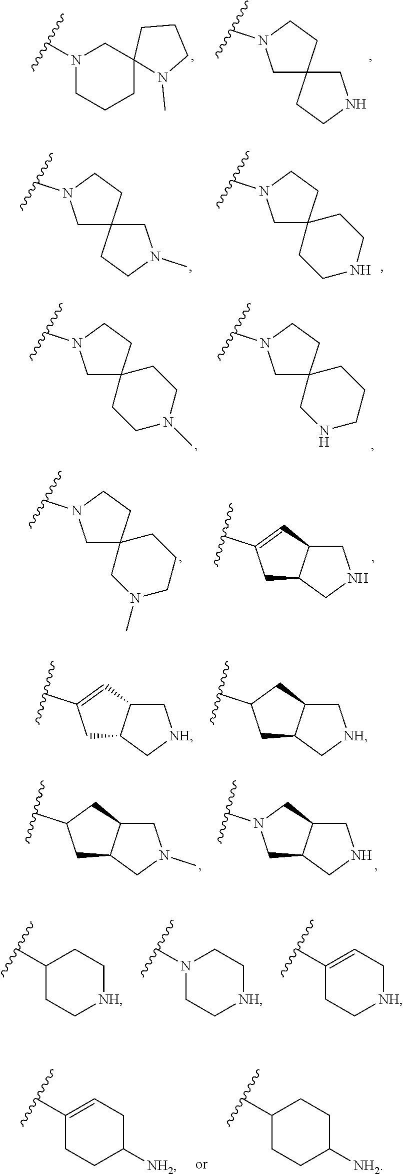

Embodiment 89. The compound of any one of embodiments 1-54, or a pharmaceutically acceptable salt thereof, wherein E is

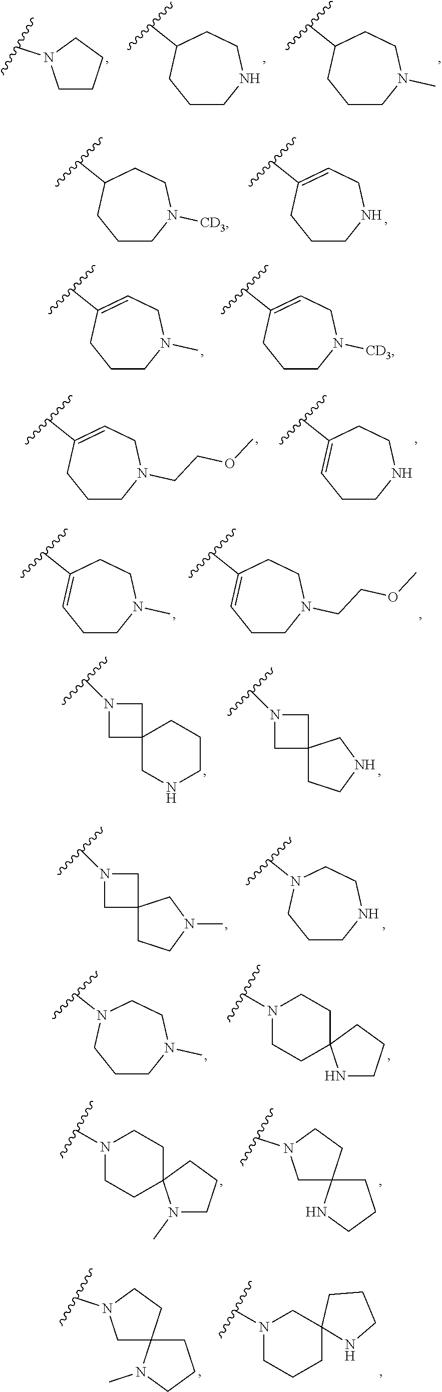

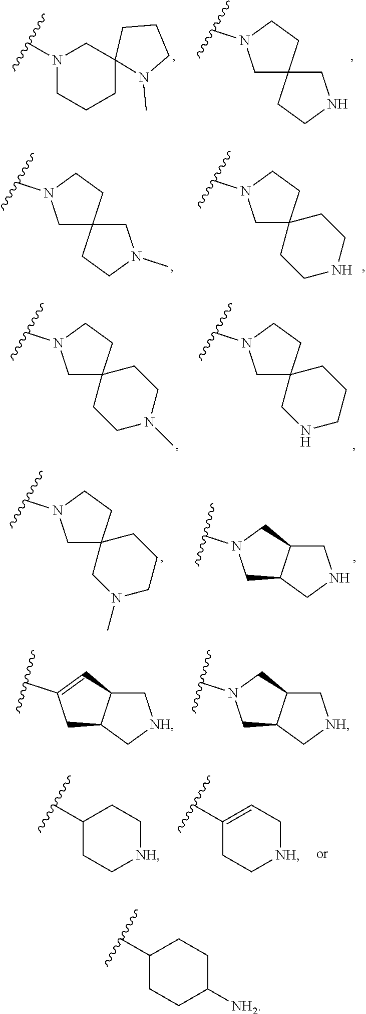

Embodiment 90. The compound of any one of embodiments 1-54, or a pharmaceutically acceptable salt thereof, wherein E is

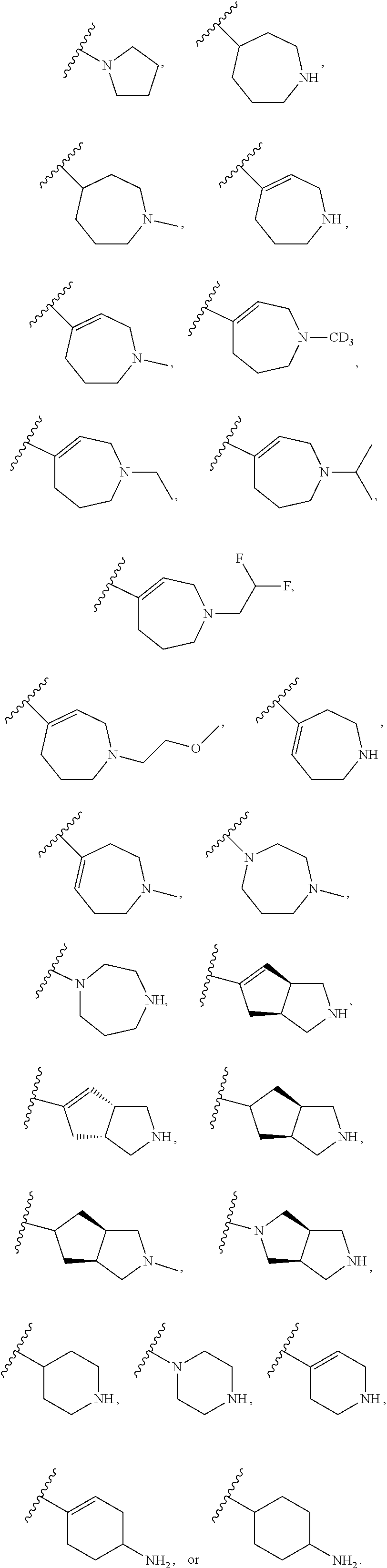

Embodiment 91. The compound of any one of embodiments 1-54, or a pharmaceutically acceptable salt thereof, wherein E is

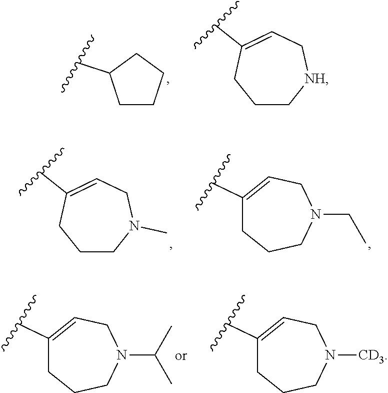

Embodiment 92. The compound of any one of embodiments 1-54, or a pharmaceutically acceptable salt thereof, wherein E is

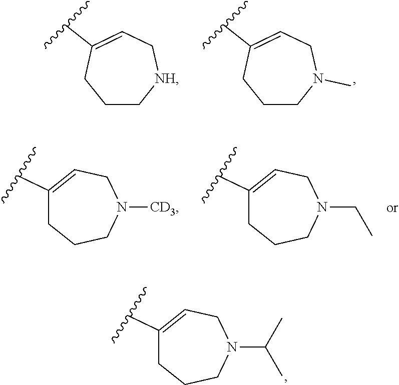

Embodiment 93. The compound of any one of embodiments 1-54, or a pharmaceutically acceptable salt thereof, wherein E is

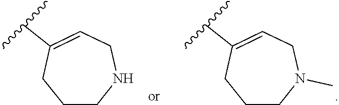

Embodiment 94. The compound of any one of embodiments 1-54, or a pharmaceutically acceptable salt thereof, wherein E is

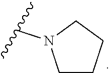 [SMILES: *N1CCCC1]

Embodiment 95. The compound of any one of embodiments 1-54, or a pharmaceutically acceptable salt thereof, wherein E is

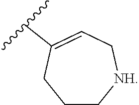 [SMILES: *C1=CCNCCC1]

Embodiment 96. The compound of any one of embodiments 1-54, or a pharmaceutically acceptable salt thereof, wherein E is

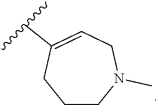 [SMILES: *C1=CCN(C)CCC1]

Embodiment 97. The compound of any one of embodiments 1-54, or a pharmaceutically acceptable salt thereof, wherein E is

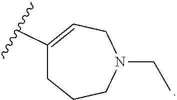 [SMILES: *C1=CCN(CC)CCC1]

Embodiment 98. The compound of any one of embodiments 1-54, or a pharmaceutically acceptable salt thereof, wherein E is

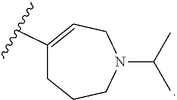 [SMILES: *C1=CCN(C(C)C)CCC1]

Embodiment 99. The compound of any one of embodiments 1-54, or a pharmaceutically acceptable salt thereof, wherein E is

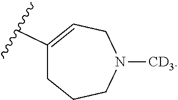 [SMILES: *C1=CCN(C([2H])([2H])[2H])CCC1]

Embodiment 100. The compound of any one of embodiments 1-54, or a pharmaceutically acceptable salt thereof, wherein E is

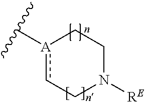

wherein
[symbol] is a single bond and A is CH or N; or
[symbol] is a double bond and A is C;
E is optionally substituted by 1-3 additional R^{E};
n is 1 or 2; and
n′ is 1 or 2.

Embodiment 101. The compound of any one of embodiments 1-86 and 100, or a pharmaceutically acceptable salt thereof, wherein each R^{E} is independently selected from halogen, C₁-C₆ alkyl, C₁-C₆ haloalkyl, C₁-C₆ alkoxy, C₁-C₆ heteroalkyl, C₁-C₆ hydroxyalkyl, NH₂ and OH, wherein R^{E} is optionally substituted with 1-5 deuteriums.

Embodiment 102. The compound of any one of embodiments 1-86 and 100, wherein each R^{E} is independently selected from halogen, C₁-C₆ alkyl, C₁-C₆ haloalkyl, C₁-C₆ alkoxy, and OH

Embodiment 103. The compound of any one of embodiments 1-86 and 100, or a pharmaceutically acceptable salt thereof, wherein each R^{E} is independently selected from halogen, C₁-C₆ alkyl, C₁-C₆ haloalkyl and OH, wherein R^{E} is optionally substituted with 1-5 deuteriums.

Embodiment 104. The compound of any one of embodiments 1-86 and 100, or a pharmaceutically acceptable salt thereof, wherein each R^{E} is independently selected from C₁-C₆ alkyl and OH, wherein R^{E} is optionally substituted with 1-5 deuteriums.

Embodiment 105. The compound of any one of embodiments 1-86 and 100, or a pharmaceutically acceptable salt thereof, wherein each R^{E} is independently selected from halogen, C₁-C₆ alkyl and OH, wherein R^{E} is optionally substituted with 1-5 deuteriums.

Embodiment 106. The compound of any one of embodiments 1-86 and 100, or a pharmaceutically acceptable salt thereof, wherein each R^{E} is independently selected from Me, CD₃, Et, iPr, F, OH, OMe, CH₂OH, CH₂CHF₂, CHF₂, CH₂F, CH₂CH₂OMe and NH₂.

Embodiment 107. The compound of any one of embodiments 1-86 and 100, or a pharmaceutically acceptable salt thereof, wherein each R^{E} is independently selected from Me, CD₃, Et, F and OH.

Embodiment 108. The compound of any one of embodiments 1-86 and 100, or a pharmaceutically acceptable salt thereof, wherein each R^{E} is independently selected from Me, CD₃ and OH.

Embodiment 109. The compound of any one of embodiments 1-860 and 100, or a pharmaceutically acceptable salt thereof, wherein each R^{E} is independently selected from Me and OH.

Embodiment 110. The compound of any one of embodiments 1-86 and 100, or a pharmaceutically acceptable salt thereof, wherein each R^{E} is independently F.

Embodiment 111. The compound of any one of embodiments 1-86 and 100, or a pharmaceutically acceptable salt thereof, wherein each R^{E} is independently CD₃.

Embodiment 112. The compound of any one of embodiments 1-86 and 100, or a pharmaceutically acceptable salt thereof, wherein each R^{E} is independently Me.

Embodiment 113. The compound of any one of embodiments 1-86 and 100, or a pharmaceutically acceptable salt thereof, wherein each R^{E} is independently OH.

Embodiment 114. The compound of any one of embodiments 1-86 and 101-113, or a pharmaceutically acceptable salt thereof, wherein R^{E} is attached to a carbon atom.

Embodiment 115. The compound of any one of embodiments 1-114, or a pharmaceutically acceptable salt thereof, wherein R¹ is selected from —O—CH₂—CH₂—CH₂-E, —O—CH₂—CH₂-E and E.

Embodiment 116. The compound of any one of embodiments 1-114, or a pharmaceutically acceptable salt thereof, wherein R¹ is —O—CH₂—CH₂—CH₂-E.

Embodiment 117. The compound of one of embodiments 1-49, or a pharmaceutically acceptable salt thereof, wherein R¹ is selected from

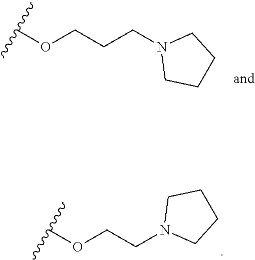 [SMILES: *OCCCN1CCCC1 ; *OCCN1CCCC1]

Embodiment 118. The compound of embodiment 116, or a pharmaceutically acceptable salt thereof, wherein R¹ is

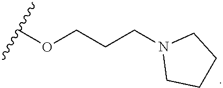 [SMILES: *OCCCN1CCCC1]

Embodiment 119. The compound of one of embodiments 1-49 and 101-114, or a pharmaceutically acceptable salt thereof, wherein R¹ is selected from

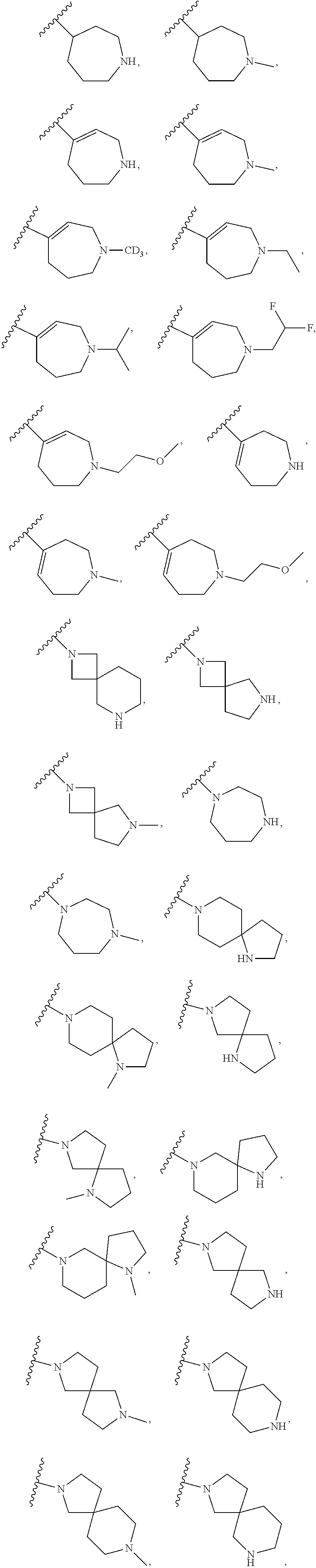

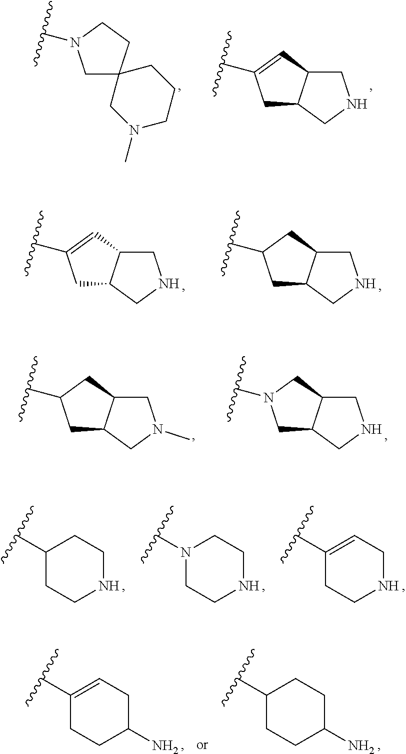

optionally substituted with 1-3 R^{E}.

Embodiment 120. The compound of any one of embodiments 1-49 and 101-114, or a pharmaceutically acceptable salt thereof, wherein R¹ is

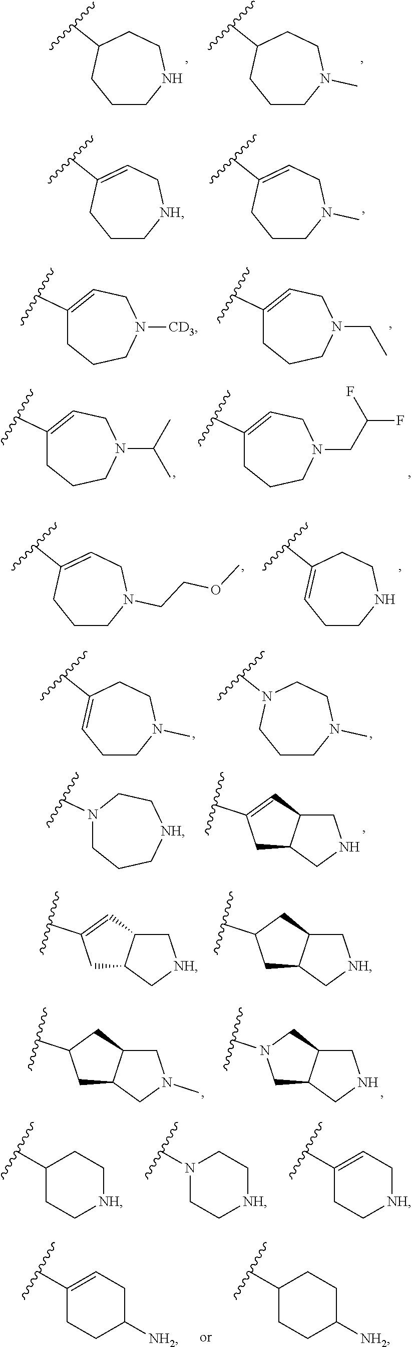

optionally substituted with 1-3 R^{E}.

Embodiment 121. The compound of any one of embodiments 1-49 and 101-114, or a pharmaceutically acceptable salt thereof, wherein R¹ is

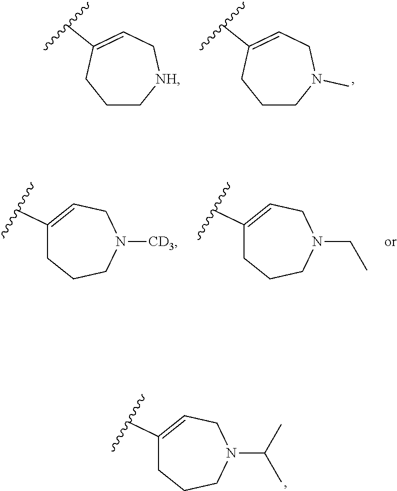

optionally substituted with 1-3 R^{E}.

Embodiment 122. The compound of any one of embodiments 1-49 and 101-114, or a pharmaceutically acceptable salt thereof, wherein R¹ is

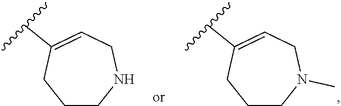

optionally substituted with 1-3 R^{E}.

Embodiment 123. The compound of any one of embodiments 1-49 and 101-114, or a pharmaceutically acceptable salt thereof, wherein R¹ is

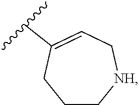 [SMILES: *C1=CCNCCC1]

optionally substituted with 1-3 R^{E}.

Embodiment 124. The compound of any one of embodiments 1-49 and 101-114, or a pharmaceutically acceptable salt thereof, wherein R¹ is

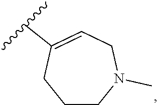 [SMILES: *C1=CCN(C)CCC1]

optionally substituted with 1-3 R^{E}.

Embodiment 125. The compound of any one of embodiments 1-49 and 101-114, or a pharmaceutically acceptable salt thereof, wherein R¹ is

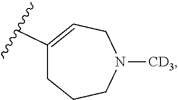 [SMILES: *C1=CCN(C([2H])([2H])[2H])CCC1]

optionally substituted with 1-3 R^{E}.

Embodiment 126. The compound of any one of embodiments 1-49 and 101-114, or a pharmaceutically acceptable salt thereof, wherein R¹ is

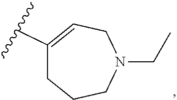 [SMILES: *C1=CCN(CC)CCC1]

optionally substituted with 1-3 R^{E}.

Embodiment 127. The compound of any one of embodiments 1-49 and 101-114, or a pharmaceutically acceptable salt thereof, wherein R¹ is

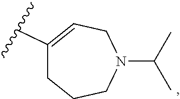 [SMILES: *C1=CCN(C(C)C)CCC1]

optionally substituted with 1-3 R^{E}.

Embodiment 128. The compound of any one of embodiments 1-49, or a pharmaceutically acceptable salt thereof, wherein R¹ is

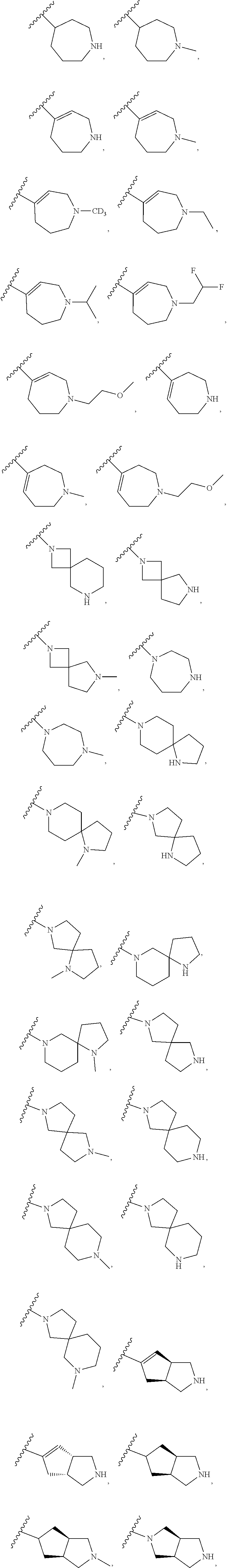

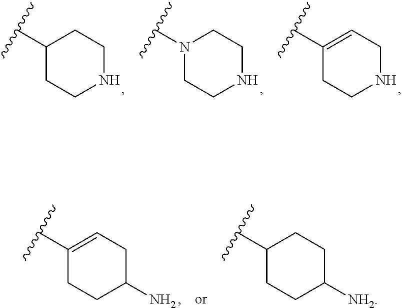 [SMILES: *C1CCNCC1 ; *N1CCNCC1 ; *C1=CCNCC1 ; *C1=CCC(N)CC1 ; *C1CCC(N)CC1]

Embodiment 129. The compound of any one of embodiments 1-49, or a pharmaceutically acceptable salt thereof, wherein R¹ is

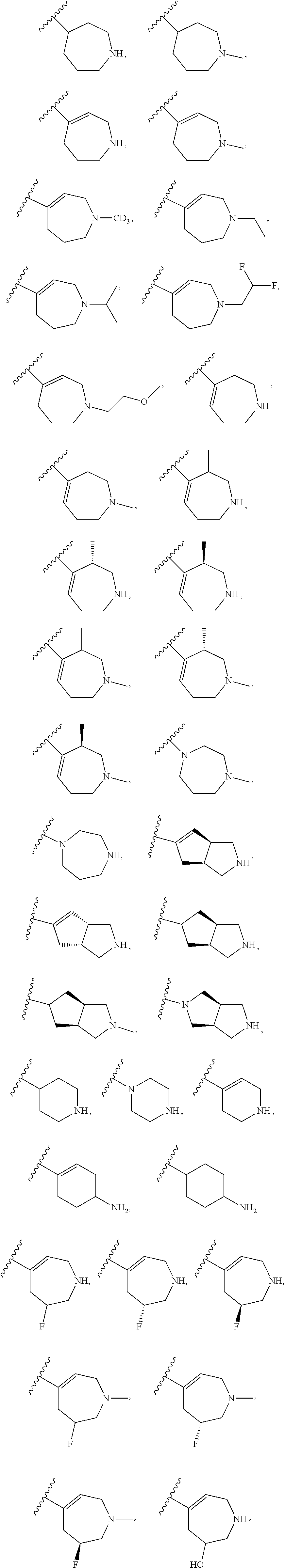

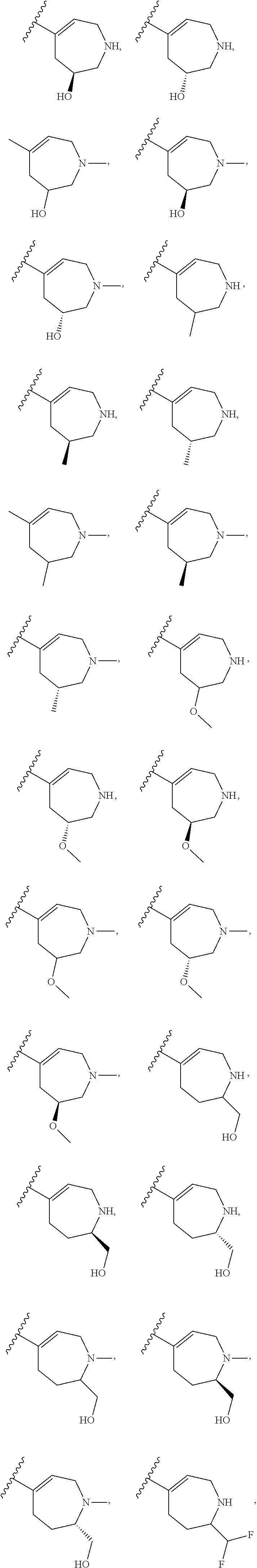

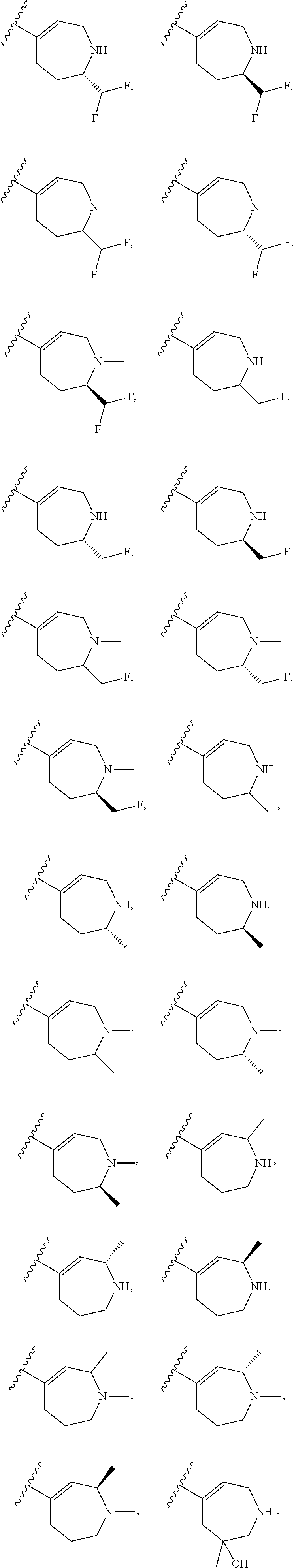

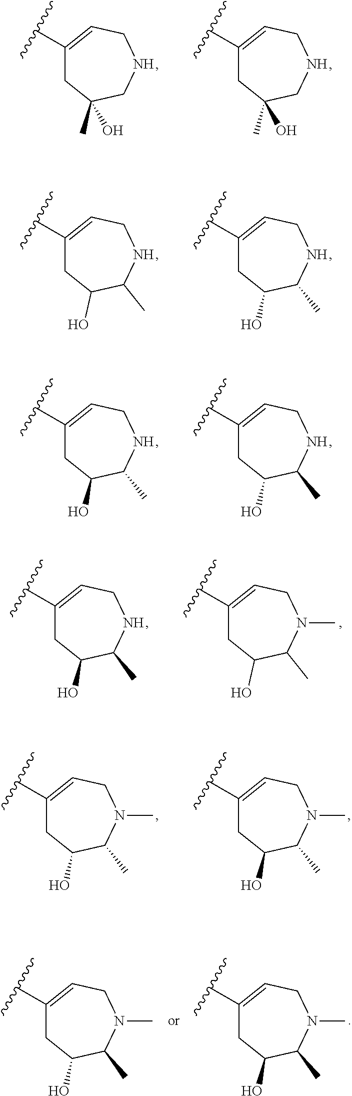

Embodiment 130. The compound of any one of embodiments 1-49, or a pharmaceutically acceptable salt thereof, wherein R¹ is

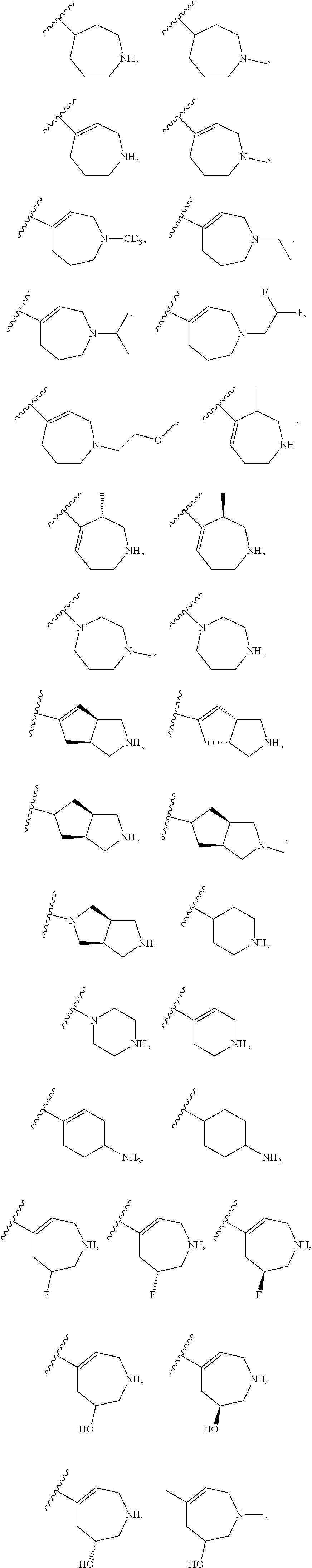

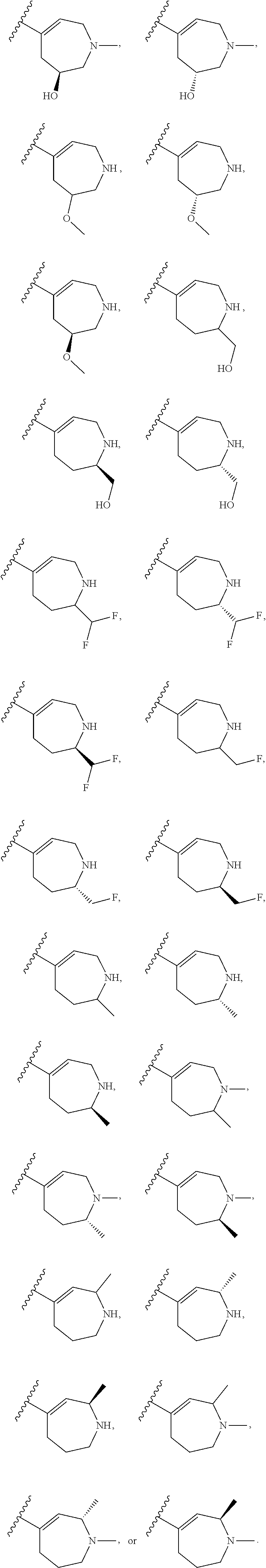

Embodiment 131. The compound of any one of embodiments 1-49, or a pharmaceutically acceptable salt thereof, wherein R¹ is

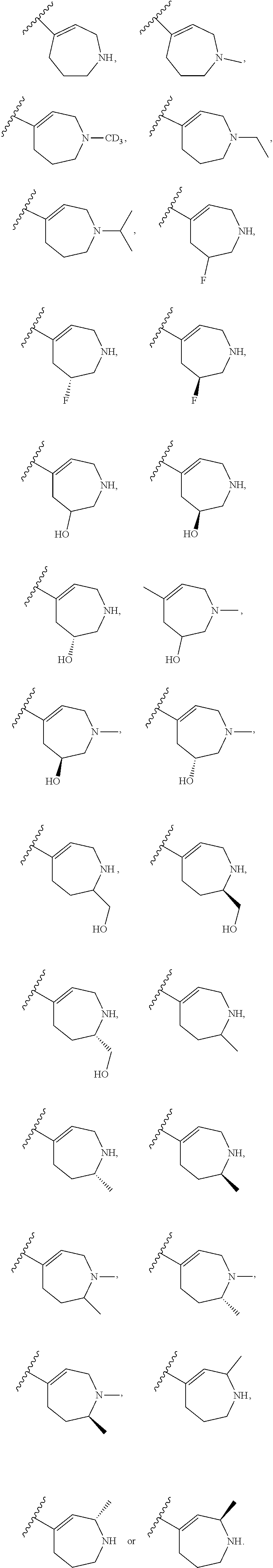

Embodiment 132. The compound of any one of embodiments 1-49, or a pharmaceutically acceptable salt thereof, wherein R¹ is

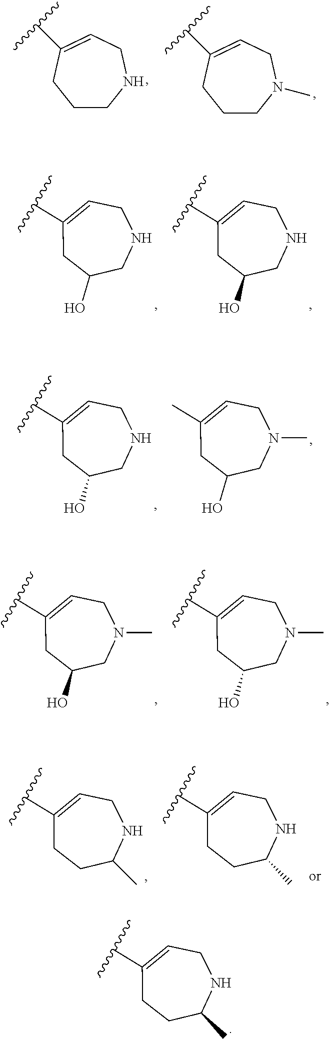

Embodiment 133. The compound of any one of embodiments 1-49, or a pharmaceutically acceptable salt thereof, wherein R¹ is

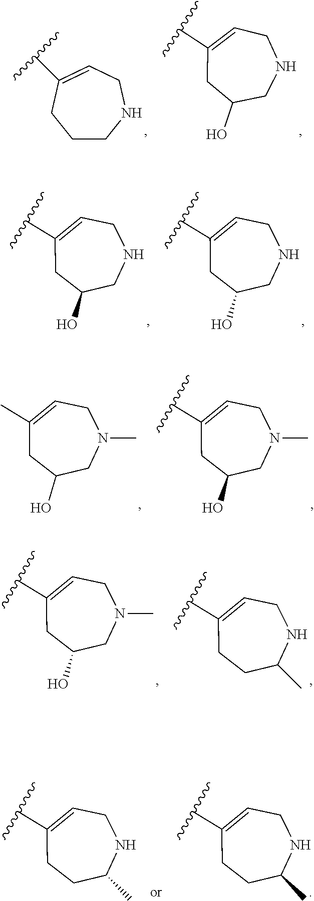

Embodiment 134. The compound of any one of embodiments 1-49, or a pharmaceutically acceptable salt thereof, wherein R¹ is

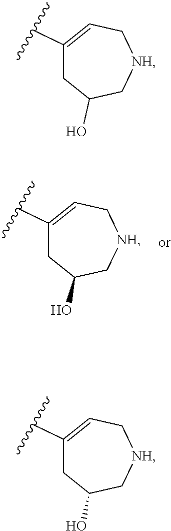 [SMILES: *C1=CCNCC(O)C1 ; *C1=CCNC[C@@H](O)C1 ; *C1=CCNC[C@H](O)C1]

Embodiment 135. The compound of any one of embodiments 1-49, or a pharmaceutically acceptable salt thereof, wherein R¹ is

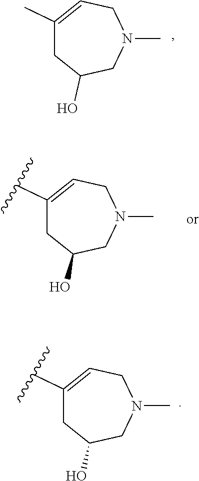

Embodiment 136. The compound of any one of embodiments 1-49, or a pharmaceutically acceptable salt thereof, wherein R¹ is

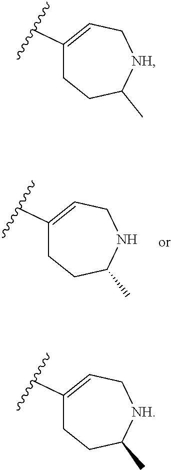

Embodiment 137. The compound of any one of embodiments 1-49, or a pharmaceutically acceptable salt thereof, wherein R¹ is

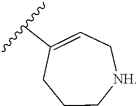 [SMILES: *C1=CCNCCC1]

Embodiment 138. The compound of any one of embodiments 1-49, or a pharmaceutically acceptable salt thereof, wherein R¹ is

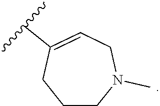 [SMILES: *C1=CCN(C)CCC1]

Embodiment 139. The compound of any one of embodiments 1-49, or a pharmaceutically acceptable salt thereof, wherein R¹ is

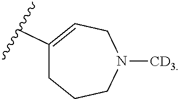 [SMILES: *C1=CCN(C([2H])([2H])[2H])CCC1]

Embodiment 140. The compound of any one of embodiments 1-49, or a pharmaceutically acceptable salt thereof, wherein R¹ is

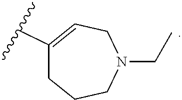 [SMILES: *C1=CCN(CC)CCC1]

Embodiment 141. The compound of any one of embodiments 1-49, or a pharmaceutically acceptable salt thereof, wherein R¹ is

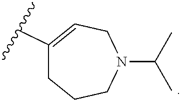 [SMILES: *C1=CCN(C(C)C)CCC1]

Embodiment 142. The compound of any one of embodiments 1-141, or a pharmaceutically acceptable salt thereof, wherein each R² and R³ is independently H, C₁-C₆ alkyl, C₁-C₆ haloalkyl, C₁-C₆ heteroalkyl, hydroxy, cyano, or halogen.

Embodiment 143. The compound of any one of embodiments 1-141, or a pharmaceutically acceptable salt thereof, wherein R² is C₁-C₆ alkyl, C₁-C₆ heteroalkyl, phenyl, C₃-C₇ cycloalkyl, heterocyclyl, C₁-C₆ alkylene-phenyl, C₁-C₆ alkylene- C₃-C₇ cycloalkyl, C₁-C₆ alkylene- heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E}.

Embodiment 144. The compound of any one of embodiments 1-141, or a pharmaceutically acceptable salt thereof, wherein R² is C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl or heteroalkyl is optionally substituted with 1-4 groups independently selected from halogen and OH, and wherein each phenyl, cycloalkyl, or heterocyclyl is optionally substituted with 1-4 R^{E}

Embodiment 145. The compound of any one of embodiments 1-141, or a pharmaceutically acceptable salt thereof, wherein R² is H, C₁-C₆ alkyl, C₁-C₆ haloalkyl, C₁-C₆ heteroalkyl, C₁-C₆ alkoxy, hydroxy, cyano, or halogen.

Embodiment 146. The compound of any one of embodiments 1-141, or a pharmaceutically acceptable salt thereof, wherein R² is C₁-C₆ alkyl, C₁-C₆ haloalkyl, C₁-C₆ heteroalkyl, C₁-C₆ alkoxy, hydroxy, cyano, or halogen.

Embodiment 147. The compound of any one of embodiments 1-141, or a pharmaceutically acceptable salt thereof, wherein R² is H, C₁-C₆ alkyl or halogen.

Embodiment 148. The compound of any one of embodiments 1-141, or a pharmaceutically acceptable salt thereof, wherein R² is C₁-C₆ alkyl or halogen.

Embodiment 149. The compound of any one of embodiments 1-141, or a pharmaceutically acceptable salt thereof, wherein R² is H.

Embodiment 150. The compound of any one of embodiments 1-141, or a pharmaceutically acceptable salt thereof, wherein R² is C₁-C₆ alkyl.

Embodiment 151. The compound of any one of embodiments 1-141, or a pharmaceutically acceptable salt thereof, wherein R² is halogen.

Embodiment 152. The compound of any one of embodiments 1-141, or a pharmaceutically acceptable salt thereof, wherein R² is selected from H, Me, F and Cl.

Embodiment 153. The compound of any one of embodiments 1-141, or a pharmaceutically acceptable salt thereof, wherein R² is selected from Me, F and Cl.

Embodiment 154. The compound of any one of embodiments 1-141, or a pharmaceutically acceptable salt thereof, wherein R² is selected from F and Cl.

Embodiment 155. The compound of any one of embodiments 1-141, or a pharmaceutically acceptable salt thereof, wherein R² is Me.

Embodiment 156. The compound of any one of embodiments 1-141, or a pharmaceutically acceptable salt thereof, wherein R² is Cl.

Embodiment 157. The compound of any one of embodiments 1-141, or a pharmaceutically acceptable salt thereof, wherein R² is F.

Embodiment 158. The compound of any one of embodiments 1-157, or a pharmaceutically acceptable salt thereof, wherein R³ is H.

Embodiment 159. The compound of any one of embodiments 1-141, or a pharmaceutically acceptable salt thereof, wherein each of R² and R³ is H.

Embodiment 160. The compound of any one of embodiments 1-159, or a pharmaceutically acceptable salt thereof, wherein R⁴ is H or Me.

Embodiment 161. The compound of any one of embodiments 1-159, or a pharmaceutically acceptable salt thereof, wherein R⁴ is H.

Embodiment 162. The compound of any one of embodiments 1-161, or a pharmaceutically acceptable salt thereof, wherein R⁵ is H.

Embodiment 163. The compound of any one of embodiments 1-161, or a pharmaceutically acceptable salt thereof, wherein R⁵ is H and R⁶ is not H.

Embodiment 164. The compound of embodiment 163, or a pharmaceutically acceptable salt thereof, wherein R⁶ is C₁-C₆ alkyl, optionally substituted with one or more deuteriums.

Embodiment 165. The compound of embodiment 163, or a pharmaceutically acceptable salt thereof, wherein R⁶ is C₁-C₆ alkyl.

Embodiment 166. The compound of any one of embodiments 1-162, or a pharmaceutically acceptable salt thereof, wherein R⁶ is selected from H, C₁-C₆ alkyl, C₁-C₆ heteroalkyl, phenyl, C₃-C₇ cycloalkyl, C₃-C₁₀ heterocyclyl, C₁-C₆ alkylene-phenyl, C₁-C₆ alkylene-C₃-C₇ cycloalkyl, and C₁-C₆ alkylene-C₃-C₁₀ heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}.

Embodiment 167. The compound of any one of embodiments 1-162, or a pharmaceutically acceptable salt thereof, wherein R⁶ is selected from C₁-C₆ alkyl, C₁-C₆ heteroalkyl, C₃-C₇ cycloalkyl, C₃-C₁₀ heterocyclyl, C₁-C₆ alkylene-C₃-C₇ cycloalkyl, and C₁-C₆ alkylene-C₃-C₁₀ heterocyclyl, wherein each alkyl or heteroalkyl is optionally substituted with 1-5 substituents independently selected from deuterium, halogen and OH, and wherein each cycloalkyl, or heterocyclyl is optionally substituted with 1-4 R^{E}.

Embodiment 168. The compound of any one of embodiments 1-162, or a pharmaceutically acceptable salt thereof, wherein R⁶ is selected from C₁-C₆ alkyl and C₁-C₆ heteroalkyl, wherein each alkyl or heteroalkyl is optionally substituted with 1-5 substituents independently selected from deuterium, halogen and OH.

Embodiment 169. The compound of any one of embodiments 1-162, or a pharmaceutically acceptable salt thereof, wherein R⁶ is selected from C₁-C₆ alkyl and C₁-C₆ heteroalkyl, wherein each alkyl or heteroalkyl is optionally substituted with 1-5 instances of deuterium.

Embodiment 170. The compound of any one of embodiments 1-162, or a pharmaceutically acceptable salt thereof, wherein R⁶ is C₁-C₆ alkyl optionally substituted with 1-5 instances of deuterium.

Embodiment 171. The compound of any one of embodiments 1-162, or a pharmaceutically acceptable salt thereof, wherein R⁶ is C₁-C₆ alkyl.

Embodiment 172. The compound of any one of embodiments 1-162, or a pharmaceutically acceptable salt thereof, wherein R⁶ is selected from Me, CD₃, Et, CH₂CD₃, CH₂CH₂OMe, CH₂CH₂CH₂OMe, CH₂CF₃, cyclobutyl, cyclopentyl, cyclohexyl, oxetanyl, CH₂-tetrahydropyranyl, CH₂-tetrahydrofuran-2-yl, N-iPr-piperidin-4-yl.

Embodiment 173. The compound of any one of embodiments 1-162, or a pharmaceutically acceptable salt thereof, wherein R⁶ is selected from Me, CD₃, Et, CH₂CD₃, CH₂CH₂OMe and CH₂CH₂CH₂OMe.

Embodiment 174. The compound of any one of embodiments 1-162, or a pharmaceutically acceptable salt thereof, wherein R⁶ is selected from Me and CD₃.

Embodiment 175. The compound of any one of embodiments 1-162, or a pharmaceutically acceptable salt thereof, wherein R⁶ is Me.

Embodiment 176. The compound of any one of embodiments 1-162, or a pharmaceutically acceptable salt thereof, wherein R⁶ is CD₃.

Embodiment 177. The compound of any one of embodiments 1-162, or a pharmaceutically acceptable salt thereof, wherein R⁶ is C₃-C₇ cycloalkyl or heterocyclyl, each optionally substituted with 1-4 R^{E}.

Embodiment 178. The compound of any one of embodiments 1-161, or a pharmaceutically acceptable salt thereof, wherein R⁵ and R⁶ are each C₁-C₆ alkyl, optionally substituted with one or more deuteriums.

Embodiment 179. The compound of any one of embodiments 1-161, or a pharmaceutically acceptable salt thereof, wherein R⁵ and R⁶ are each C₁-C₆ alkyl.

Embodiment 180. The compound of any one of embodiments 1-161, or a pharmaceutically acceptable salt thereof, wherein R⁵ and R⁶ are each Me.

Embodiment 181. The compound of any one of embodiments 1-180, or a pharmaceutically acceptable salt thereof, wherein R⁷ is selected from H, C₁-C₆ alkyl, C₁-C₆ alkoxy, C₁-C₆ heteroalkyl, phenyl, C₃-C₇ cycloalkyl, C₃-C₁₀ heterocyclyl, C₁-C₆ alkylene-phenyl, C₁-C₆ alkylene- C₃-C₇ cycloalkyl, C₁-C₆ alkylene-heterocyclyl, hydroxy, cyano, CO—R^{C}, NR^{D}₂, and halogen, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}.

Embodiment 182. The compound of any one of embodiments 1-180, or a pharmaceutically acceptable salt thereof, wherein R⁷ is selected from H, C₁-C₆ alkyl, C₁-C₆ alkoxy, and halogen, wherein each alkyl is optionally substituted with 1-5 instances of deuterium.

Embodiment 183. The compound of any one of embodiments 1-180, or a pharmaceutically acceptable salt thereof, wherein R⁷ is selected from H, F, Cl, Me, Et and OMe.

Embodiment 184. The compound of any one of embodiments 1-180, or a pharmaceutically acceptable salt thereof, wherein R⁷ is selected from H, F and Me.

Embodiment 185. The compound of any one of embodiments 1-180, or a pharmaceutically acceptable salt thereof, wherein R⁷ is H or F.

Embodiment 186. The compound of any one of embodiments 1-180, or a pharmaceutically acceptable salt thereof, wherein R⁷ is H.

Embodiment 187. The compound of any one of embodiments 1-180, or a pharmaceutically acceptable salt thereof, wherein R⁷ is F.

Embodiment 188. The compound of any one of embodiments 1-187, or a pharmaceutically acceptable salt thereof, wherein R⁸ is selected from C₁-C₆ alkyl, C₃-C₇ cycloalkyl and heterocyclyl, wherein each alkyl is optionally substituted with 1-5 substituents independently selected from deuterium, halogen and OH, and wherein each cycloalkyl or heterocyclyl is optionally substituted with 1-4 R^{E}.

Embodiment 189. The compound of any one of embodiments 1-187, or a pharmaceutically acceptable salt thereof, wherein R⁸ is selected from C₁-C₆ alkyl, C₃-C₇ cycloalkyl and heterocyclyl, wherein each alkyl is optionally substituted with 1-4 substituents independently selected from halogen and OH, and wherein each cycloalkyl or heterocyclyl is optionally substituted with 1-4 R^{E}.

Embodiment 190. The compound of any one of embodiments 1-187, or a pharmaceutically acceptable salt thereof, wherein R⁸ is C₁-C₆ alkyl, optionally substituted with 1-5 deuteriums.

Embodiment 191. The compound of any one of embodiments 1-187, or a pharmaceutically acceptable salt thereof, wherein R⁸ is C₁-C₆ alkyl, optionally substituted with 1-5 deuteriums.

Embodiment 192. The compound of any one of embodiments 1-187, or a pharmaceutically acceptable salt thereof, wherein R⁸ is selected from methyl, ethyl, CH₂D, iPr, cyclopropyl, cyclohexyl and CH₂CF₃.

Embodiment 193. The compound of any one of embodiments 1-187, or a pharmaceutically acceptable salt thereof, wherein R⁸ is selected from methyl and CH₂D.

Embodiment 194. The compound of any one of embodiments 1-187, or a pharmaceutically acceptable salt thereof, wherein R⁸ is Me.

Embodiment 195. The compound of any one of embodiments 1-187, or a pharmaceutically acceptable salt thereof, wherein R⁸ is CH₂D.

Embodiment 196. The compound of any one of embodiments 5-195, or a pharmaceutically acceptable salt thereof, wherein Z is CR⁹R¹⁰ and each R⁹ and R¹⁰ is independently H, C₁-C₆ alkyl, or halogen, or R⁹ and R¹⁰ together with the carbon to which they are attached form CO.

Embodiment 197. The compound of any one of embodiments 5-195, or a pharmaceutically acceptable salt thereof, wherein Z is CR⁹R¹⁰ and each R⁹ and R¹⁰ is independently H, C₁-C₆ alkyl, or halogen.

Embodiment 198. The compound of any one of embodiments 5-195, or a pharmaceutically acceptable salt thereof, wherein Z is CR⁹R¹⁰ and each R⁹ and R¹⁰ is independently H, Me or F.

Embodiment 199. The compound of any one of embodiments 5-0, or a pharmaceutically acceptable salt thereof, wherein Z is selected from CH₂, CF₂, and CMe₂.

Embodiment 200. The compound of any one of embodiments 5-195, or a pharmaceutically acceptable salt thereof, wherein Z is selected from C═O, CF₂ and CH₂.

Embodiment 201. The compound of any one of embodiments 5-195, or a pharmaceutically acceptable salt thereof, wherein Z is CH₂.

Embodiment 202. The compound of any one of embodiments 1-201, or a pharmaceutically acceptable salt thereof, wherein each R¹² is independently selected from H, C₁-C₆ alkylene-phenyl and C₁-C₆ alkyl.

Embodiment 203. The compound of any one of embodiments 1-201, or a pharmaceutically acceptable salt thereof, wherein each R¹² is independently H or C₁-C₆ alkyl.

Embodiment 204. The compound of any one of embodiments 1-201, or a pharmaceutically acceptable salt thereof, wherein each R¹² is H.

Embodiment 205. The compound of any one of embodiments 1-204 or a pharmaceutically acceptable salt thereof, wherein each R¹¹ is independently selected from H, C₁-C₆ alkyl, hydroxy, and halogen, wherein each alkyl is optionally substituted with 1-4 substituents independently selected from halogen and OH; or two R¹¹ are taken together with the carbon to which they are attached to form CO or a spirofused C₃-C₇ cycloalkyl.

Embodiment 206. The compound of any one of embodiments 1-204, or a pharmaceutically acceptable salt thereof, wherein each R¹¹ is independently selected from H, C₁-C₆ alkyl, hydroxy, and halogen, or two R¹¹ are taken together with the carbon to which they are attached to form a spirofused C₃-C₇ cycloalkyl.

Embodiment 207. The compound of any one of embodiments 1-204, or a pharmaceutically acceptable salt thereof, wherein each R¹¹ is independently selected from H, Me, hydroxy, and F, or two R¹¹ are taken together with the carbon to which they are attached to form a spirofused cyclopropyl.

Embodiment 208. The compound of any one of embodiments 1-204, or a pharmaceutically acceptable salt thereof, wherein each R¹¹ is independently selected from H and Me.

Embodiment 209. The compound of any one of embodiments 1-204, or a pharmaceutically acceptable salt thereof, wherein each R¹¹ is independently selected from hydroxy and Me.

Embodiment 210. The compound of any one of embodiments 1-204, or a pharmaceutically acceptable salt thereof, wherein each R¹¹ is independently H.

Embodiment 211. The compound of any one of embodiments 1-204, or a pharmaceutically acceptable salt thereof, wherein each R¹¹ is independently F.

Embodiment 212. The compound of any one of embodiments 1-204, or a pharmaceutically acceptable salt thereof, wherein two R¹¹ are taken together with the carbon to which they are attached to form a spirofused cyclopropyl.

Embodiment 213. The compound of any one of embodiments 1-212, or a pharmaceutically acceptable salt thereof, wherein each R¹³ is independently selected from H, C₁-C₆ alkyl, C₁-C₆ heteroalkyl, hydroxy, cyano, and halogen, wherein each alkyl or heteroalkyl is optionally substituted with 1-4 substituents independently selected from halogen and OH, or two R¹³ are taken together with the carbon to which they are attached to form CO or a spirofused C₃-C₇ cycloalkyl.

Embodiment 214. The compound of any one of embodiments 1-212, or a pharmaceutically acceptable salt thereof, wherein each R¹³ is independently selected from H, C₁-C₆ alkyl, hydroxy, and halogen, or two R¹³ are taken together with the carbon to which they are attached to form a spirofused C₃-C₇ cycloalkyl.

Embodiment 215. The compound of any one of embodiments 1-212, or a pharmaceutically acceptable salt thereof, wherein each R¹³ is independently selected from H, Me, hydroxy, and F, or two R¹³ are taken together with the carbon to which they are attached to form a spirofused cyclopropyl.

Embodiment 216. The compound of any one of embodiments 1-212, or a pharmaceutically acceptable salt thereof, wherein each R¹³ is independently H.

Embodiment 217. The compound of any one of embodiments 1-216, or a pharmaceutically acceptable salt thereof, wherein each R^{C} is independently H, OH, N(R¹²)₂, C₁-C₆ alkyl, or C₁-C₆ alkoxy.

Embodiment 218. The compound of any one of embodiments 1-217, or a pharmaceutically acceptable salt thereof, wherein each R^{D} is independently H or C₁-C₆ alkyl.

Embodiment 219. A compound of any one of embodiments 5-15 and 50-218, or a pharmaceutically acceptable salt thereof, wherein the compound is of Formula (IIa)

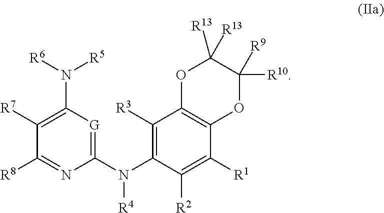

Embodiment 220. A compound of any one of embodiments 5-15 and 50-218, or a pharmaceutically acceptable salt thereof, wherein the compound is of Formula (IIb)

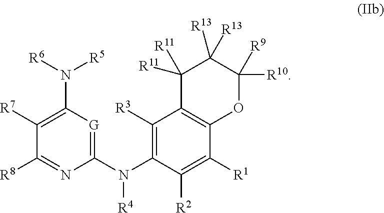

Embodiment 221. A compound of any one of embodiments 5-15 and 50-218, or a pharmaceutically acceptable salt thereof, wherein the compound is of Formula (IIc)

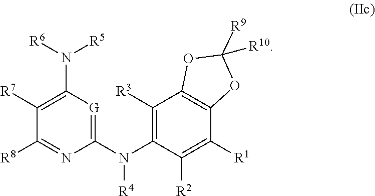

Embodiment 222. A compound of any one of embodiments 5-15 and 50-218, or a pharmaceutically acceptable salt thereof, wherein the compound is of Formula (IId)

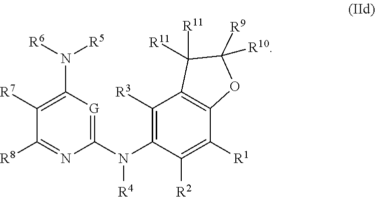

Embodiment 223. A compound of any one of embodiments 16-26 and 50-218, or a pharmaceutically acceptable salt thereof, wherein the compound is of Formula (IIIa)

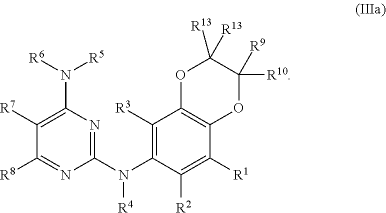

Embodiment 224. A compound of any one of embodiments 16-26 and 50-218, or a pharmaceutically acceptable salt thereof, wherein the compound is of Formula (IIb)

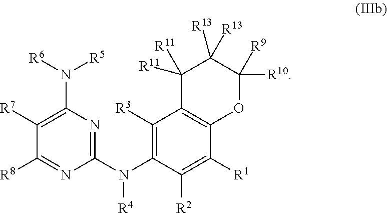

Embodiment 225. A compound of any one of embodiments 16-26 and 50-218, or a pharmaceutically acceptable salt thereof, wherein the compound is of Formula (IIc)

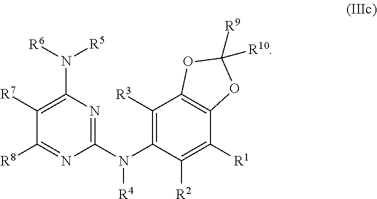

Embodiment 226. A compound of any one of embodiments 16-26 and 50-218, or a pharmaceutically acceptable salt thereof, wherein the compound is of Formula (IIId)

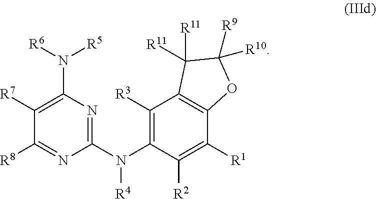

Embodiment 227. A compound of any one of embodiments 16-36 and 50-218, or a pharmaceutically acceptable salt thereof, wherein the compound is of Formula (IVa)

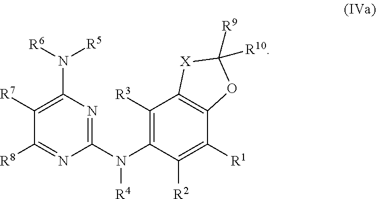

Embodiment 228. A compound of any one of embodiments 16-36 and 50-218, or a pharmaceutically acceptable salt thereof, wherein the compound is of Formula (IVb)

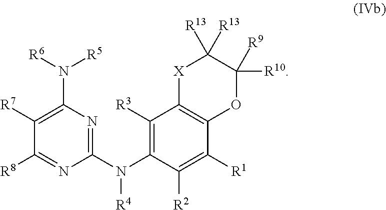

Embodiment 229. A compound of any one of embodiments 5-15, 101-113 and 142-218, or a pharmaceutically acceptable salt thereof, wherein the compound is of Formula (IIa-1)

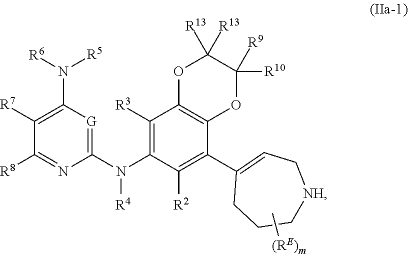

wherein m is 0, 1, 2, 3 or 4.

Embodiment 230. A compound of any one of embodiments 5-15, 101-113 and 142-218, or a pharmaceutically acceptable salt thereof, wherein the compound is of Formula (IIb-1)

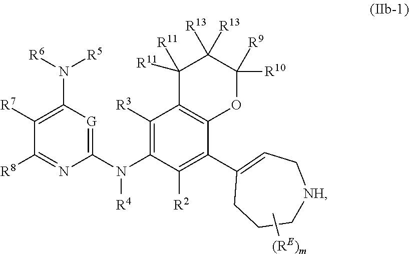

wherein m is 0, 1, 2, 3 or 4.

Embodiment 231. A compound of any one of embodiments 5-15, 101-113 and 142-218, or a pharmaceutically acceptable salt thereof, wherein the compound is of Formula (IIc-1)

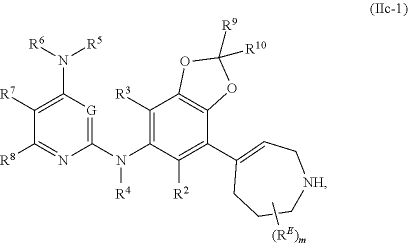

wherein m is 0, 1, 2, 3 or 4.

Embodiment 232. A compound of any one of embodiments 5-15, 101-113 and 142-218, or a pharmaceutically acceptable salt thereof, wherein the compound is of Formula (IId-1)

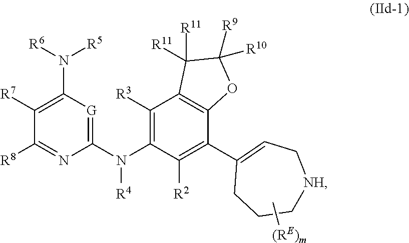

wherein m is 0, 1, 2, 3 or 4.

Embodiment 233. A compound of any one of embodiments 16-26, 101-113 and 142-218, or a pharmaceutically acceptable salt thereof, wherein the compound is of Formula (IIIa-1)

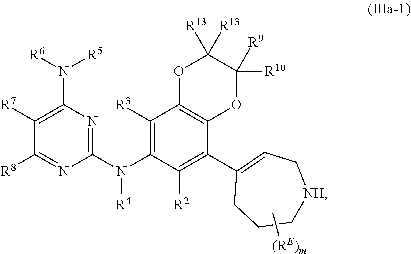

wherein m is 0, 1, 2, 3 or 4.

Embodiment 234. A compound of any one of embodiments 16-26, 101-113 and 142-218, or a pharmaceutically acceptable salt thereof, wherein the compound is of Formula (IIIb-1)

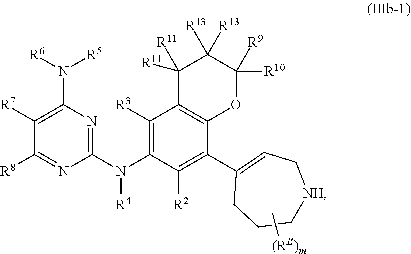

wherein m is 0, 1, 2, 3 or 4.

Embodiment 235. A compound of any one of embodiments 16-26, 101-113 and 142-218, or a pharmaceutically acceptable salt thereof, wherein the compound is of Formula (IIIc-1)

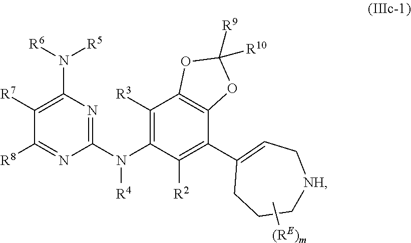

wherein m is 0, 1, 2, 3 or 4.

Embodiment 236. A compound of any one of embodiments 16-26, 101-113 and 142-218, or a pharmaceutically acceptable salt thereof, wherein the compound is of Formula (IIId-1)

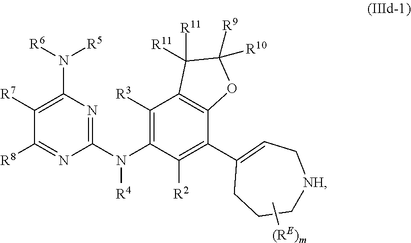

wherein m is 0, 1, 2, 3 or 4.

Embodiment 237. A compound of any one of embodiments 16-36, 101-113 and 142-218, or a pharmaceutically acceptable salt thereof, wherein the compound is of Formula (IVa-1)

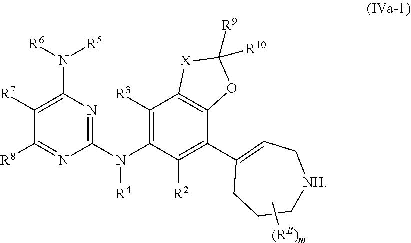

Embodiment 238. A compound of any one of embodiments 16-36, 101-113 and 142-218, or a pharmaceutically acceptable salt thereof, wherein the compound is of Formula (IVb-1)

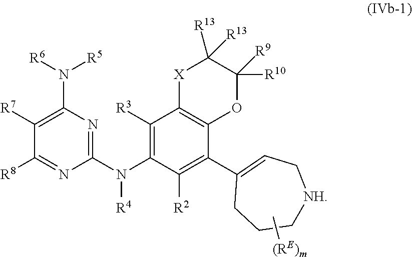

Embodiment 239. A compound of formula (IIIa-2) or a pharmaceutically acceptable salt thereof, wherein

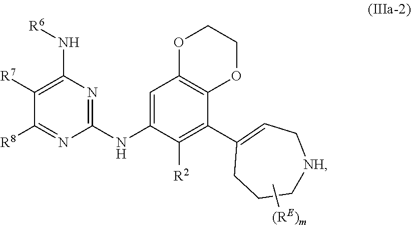

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

Embodiment 240. A compound of formula (IIIb-2) or a pharmaceutically acceptable salt thereof, wherein

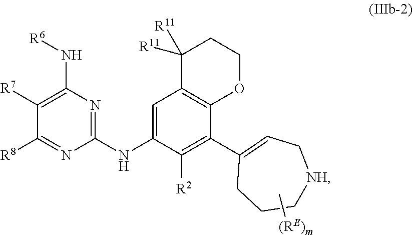

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R¹¹ is independently selected from H, C₁-C₆ alkyl, hydroxy, and halogen, or two R¹¹ are taken together with the carbon to which they are attached to form a spirofused C₃-C₇ cycloalkyl;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

Embodiment 241. A compound of formula (IVb-2) or a pharmaceutically acceptable salt thereof, wherein

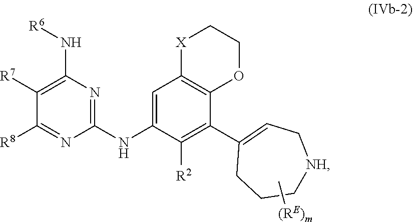

X is 0 or C(R¹¹)₂
R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R¹¹ is independently selected from H, C₁-C₆ alkyl, hydroxy, and halogen, or two R¹¹ are taken together with the carbon to which they are attached to form a spirofused C₃-C₇ cycloalkyl;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

Embodiment 242. A compound of formula (IIIc-2) or a pharmaceutically acceptable salt thereof, wherein

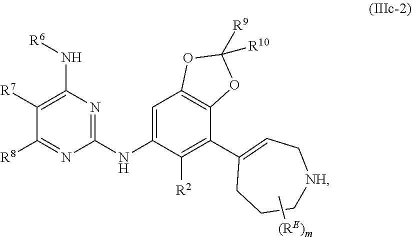

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R⁹ and R¹⁰ is independently H, C₁-C₆ alkyl, or halogen;
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

Embodiment 243. A compound of formula (IIId-2) or a pharmaceutically acceptable salt thereof, wherein

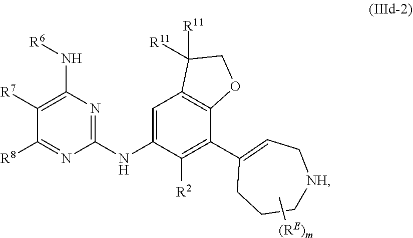

R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R¹¹ is independently selected from H, C₁-C₆ alkyl, hydroxy, and halogen, or two R¹¹ are taken together with the carbon to which they are attached to form a spirofused C₃-C₇ cycloalkyl
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

Embodiment 244. A compound of formula (IVa-2) or a pharmaceutically acceptable salt thereof, wherein

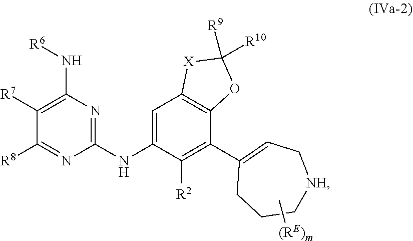

X is O or C(R¹¹)₂;
R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R⁹ and R¹⁰ is independently H, C₁-C₆ alkyl, or halogen;
each R¹¹ is independently selected from H, C₁-C₆ alkyl, hydroxy, and halogen, or two R¹¹ are taken together with the carbon to which they are attached to form a spirofused C₃-C₇ cycloalkyl
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

Embodiment 245. The compound of any one of embodiments 240, 241, 243 and 244, or a pharmaceutically acceptable salt thereof, wherein each R¹¹ is independently selected from H, Me, hydroxy, and F, or two R¹¹ are taken together with the carbon to which they are attached to form a spirofused cyclopropyl.

Embodiment 246. The compound of any one of embodiments 240, 241, 243 and 244, or a pharmaceutically acceptable salt thereof, wherein each R¹¹ is independently selected from H and Me.

Embodiment 247. The compound of any one of embodiments 240, 241, 243 and 244, or a pharmaceutically acceptable salt thereof, wherein each R¹¹ is H.

Embodiment 248. The compound of embodiment 242 or 244, or a pharmaceutically acceptable salt thereof, wherein each R⁹ and R¹⁰ is independently H, Me or F.

Embodiment 249. The compound of embodiment 242 or 244, or a pharmaceutically acceptable salt thereof, wherein each R⁹ and R¹⁰ is independently H.

Embodiment 250. The compound of any one of embodiments 239-249, or a pharmaceutically acceptable salt thereof, wherein R² is selected from C₁-C₆ alkyl and halogen.

Embodiment 251. The compound of any one of embodiments 239-249, or a pharmaceutically acceptable salt thereof, wherein R² is selected from H, Me, F and Cl.

Embodiment 252. The compound of any one of embodiments 239-249, or a pharmaceutically acceptable salt thereof, wherein R² is selected from Me, F and Cl.

Embodiment 253. The compound of any one of embodiments 239-249, or a pharmaceutically acceptable salt thereof, wherein R² is H Embodiment 254. The compound of any one of embodiments 239-249, or a pharmaceutically acceptable salt thereof, wherein R² is Me.

Embodiment 255. The compound of any one of embodiments 239-249, or a pharmaceutically acceptable salt thereof, wherein R² is F.

Embodiment 256. The compound of any one of embodiments 239-249, or a pharmaceutically acceptable salt thereof, wherein R² is Cl.

Embodiment 257. The compound of any one of embodiments 239-256, or a pharmaceutically acceptable salt thereof, wherein R⁶ is selected from Me, CD₃, Et, CH₂CD₃, CH₂CH₂OMe and CH₂CH₂CH₂OMe.

Embodiment 258. The compound of any one of embodiments 239-256, or a pharmaceutically acceptable salt thereof, wherein R⁶ is selected from Me and CD₃.

Embodiment 259. The compound of any one of embodiments 239-256, or a pharmaceutically acceptable salt thereof, wherein R⁶ is Me.

Embodiment 260. The compound of any one of embodiments 239-256, or a pharmaceutically acceptable salt thereof, wherein R⁶ is CD₃.

Embodiment 261. The compound of any one of embodiments 239-260, or a pharmaceutically acceptable salt thereof, wherein R⁷ is selected from H, F, Cl, OMe and Me.

Embodiment 262. The compound of any one of embodiments 239-260, or a pharmaceutically acceptable salt thereof, wherein R⁷ is selected from H, F and Me.

Embodiment 263. The compound of any one of embodiments 239-260, or a pharmaceutically acceptable salt thereof, wherein R⁷ is H.

Embodiment 264. The compound of any one of embodiments 239-260, or a pharmaceutically acceptable salt thereof, wherein R⁷ is F.

Embodiment 265. The compound of any one of embodiments 239-260, or a pharmaceutically acceptable salt thereof, wherein R⁷ is Me.

Embodiment 266. The compound of any one of embodiments 239-265, or a pharmaceutically acceptable salt thereof, wherein R⁸ is selected from Me and CH₂D.

Embodiment 267. The compound of any one of embodiments 239-265, or a pharmaceutically acceptable salt thereof, wherein R⁸ is Me.

Embodiment 268. The compound of any one of embodiments 239-265, or a pharmaceutically acceptable salt thereof, wherein R⁸ is CH₂D.

Embodiment 269. The compound of any one of embodiments 239-268, or a pharmaceutically acceptable salt thereof, wherein each R^{E} is independently selected from halogen, C₁-C₆ alkyl, and OH, wherein R^{E} is optionally substituted with 1-5 deuteriums.

Embodiment 270. The compound of any one of embodiments 239-268, or a pharmaceutically acceptable salt thereof, wherein each R^{E} is independently selected from F, Me, Et, CD₃, and OH.

Embodiment 271. The compound of any one of embodiments 239-268, or a pharmaceutically acceptable salt thereof, wherein each R^{E} is independently selected from Me and OH.

Embodiment 272. The compound of any one of embodiments 239-268, or a pharmaceutically acceptable salt thereof, wherein each R^{E} is independently Me.

Embodiment 273. The compound of any one of embodiments 239-268, or a pharmaceutically acceptable salt thereof, wherein each R^{E} is independently OH.

Embodiment 274. The compound of any one of embodiments 239-268, or a pharmaceutically acceptable salt thereof, wherein R^{E} is attached to a carbon atom.

Embodiment 275. The compound of any one of embodiments 239-268, or a pharmaceutically acceptable salt thereof, wherein m is 0.

Embodiment 276. The compound of any one of embodiments 239-274, or a pharmaceutically acceptable salt thereof, wherein m is 1, 2 or 3.

Embodiment 277. The compound of any one of embodiments 239-274, or a pharmaceutically acceptable salt thereof, wherein m is 1 or 2.

Embodiment 278. The compound of any one of embodiments 2390-274, or a pharmaceutically acceptable salt thereof, wherein m is 1.

Embodiment 279. The compound of any one of embodiments 239-274, or a pharmaceutically acceptable salt thereof, wherein m is 2.

Embodiment 280. The compound of any one of embodiments 239-274, or a pharmaceutically acceptable salt thereof, wherein m is 3.

Embodiment 281. The compound of any one of embodiments 239-274, or a pharmaceutically acceptable salt thereof, wherein m is 4.

Embodiment 282. The compound of embodiment 4, or a pharmaceutically acceptable salt thereof, wherein the compound is selected from the group consisting of:

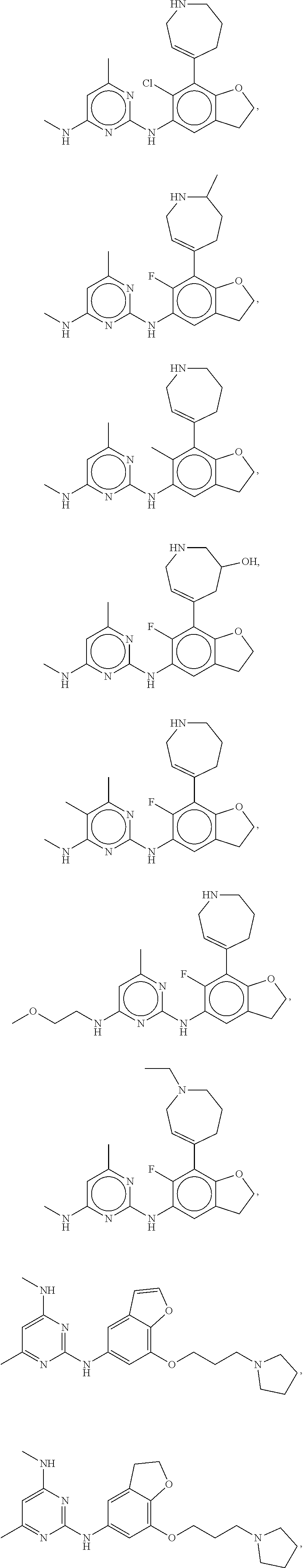

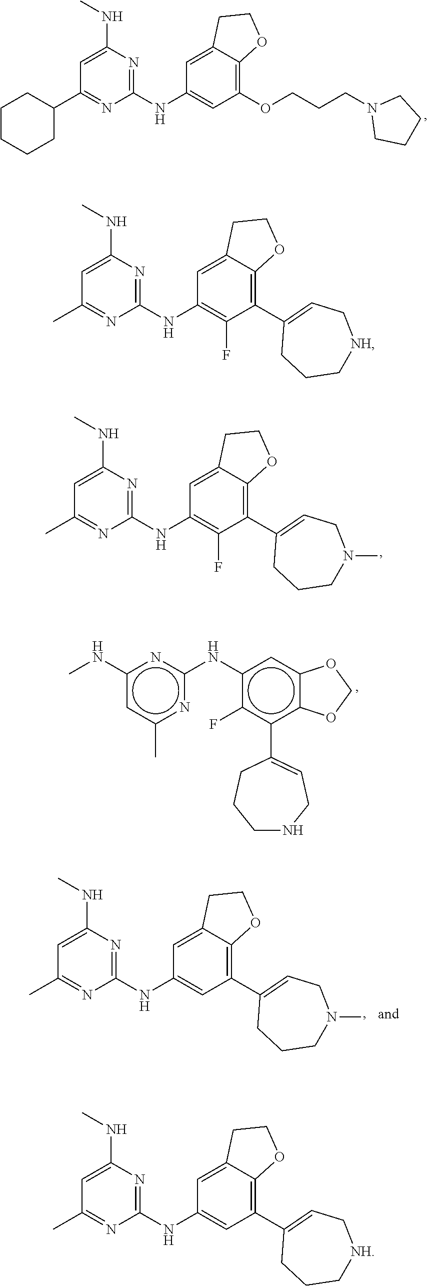

Embodiment 283. The compound of embodiment 3, or a pharmaceutically acceptable salt thereof, wherein the compound is selected from the group consisting of:

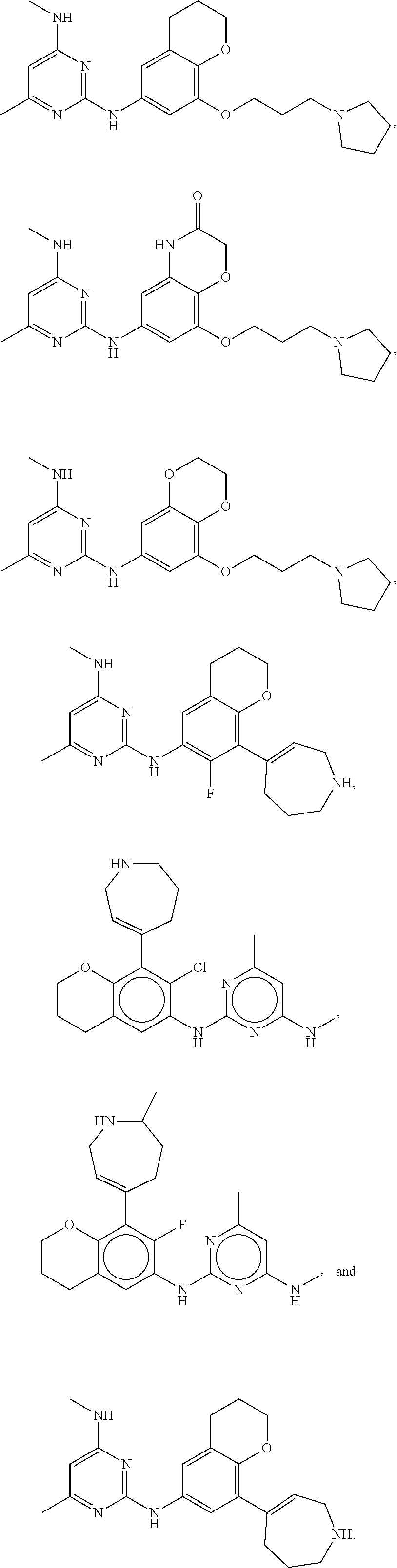

Embodiment 284. The compound of any one of embodiments 1, 4, 5-12, 16-26 and 239-241, or a pharmaceutically acceptable salt thereof, wherein the compound is selected from:

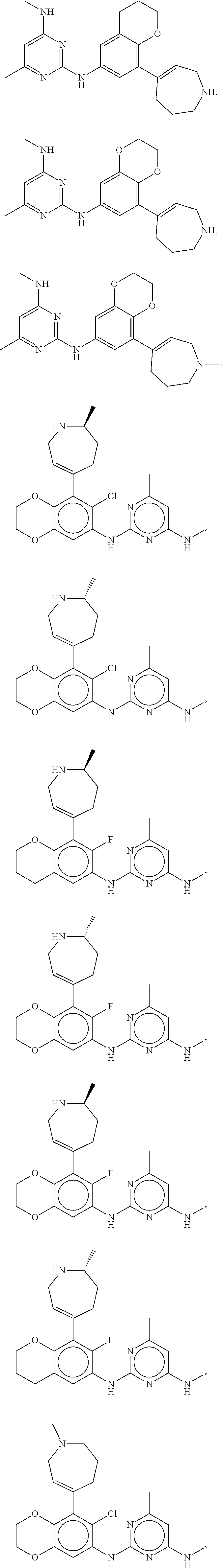

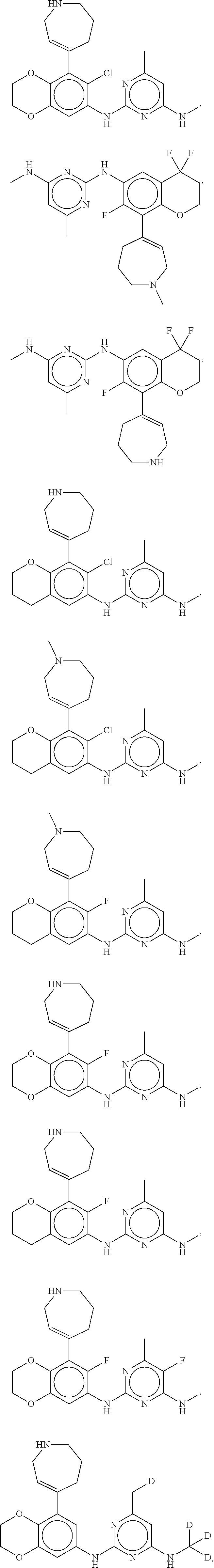

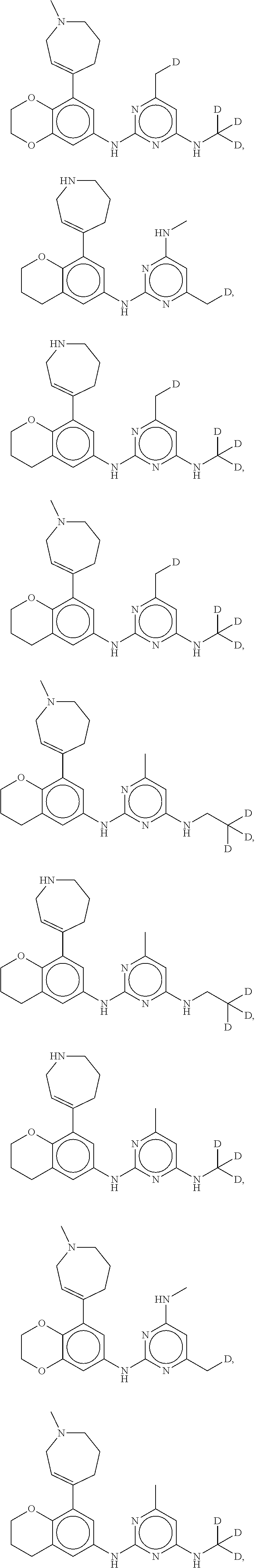

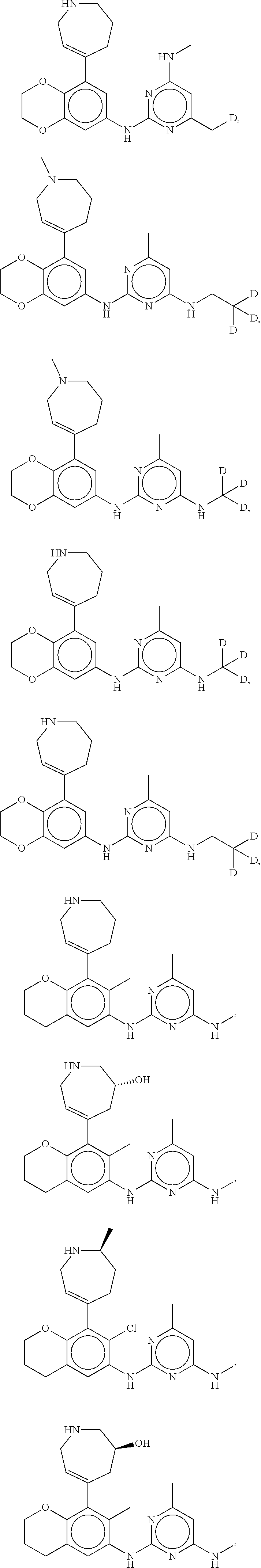

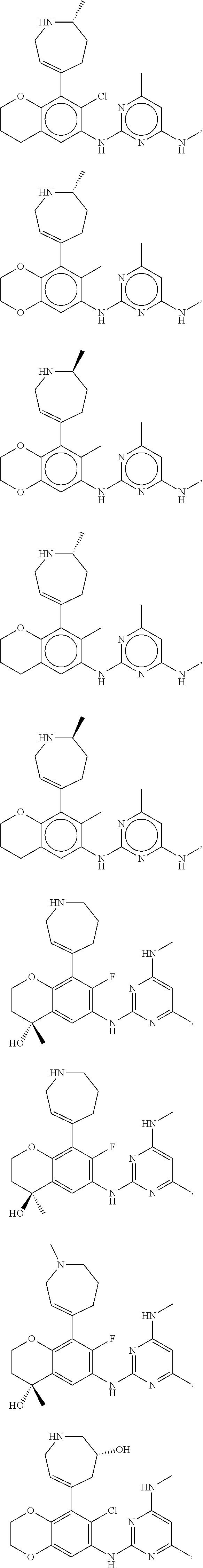

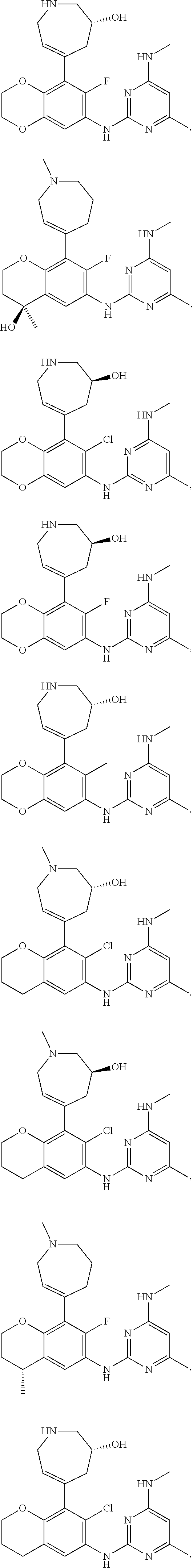

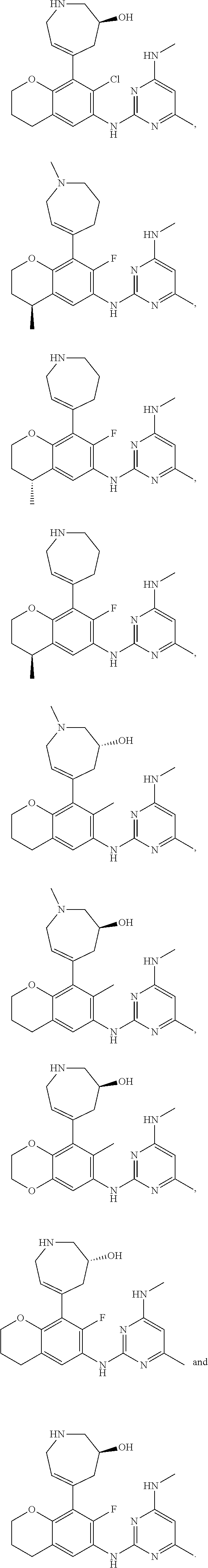

Embodiment 285. The compound of any one of embodiments 1, 4, 5-12, 16-26 and 239-241, or a pharmaceutically acceptable salt thereof, wherein the compound is selected from:

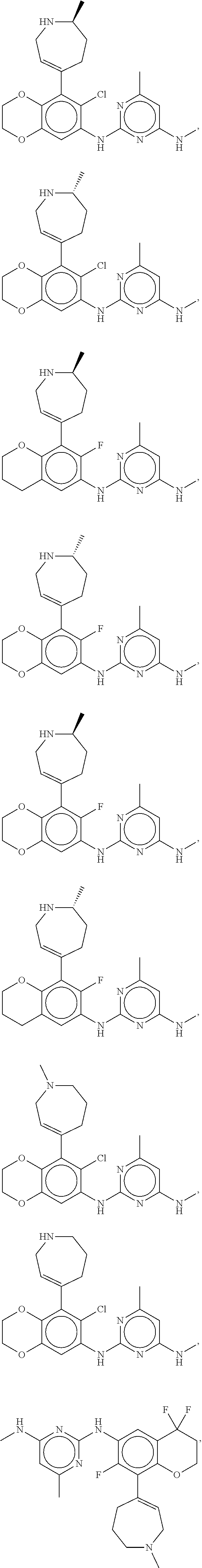

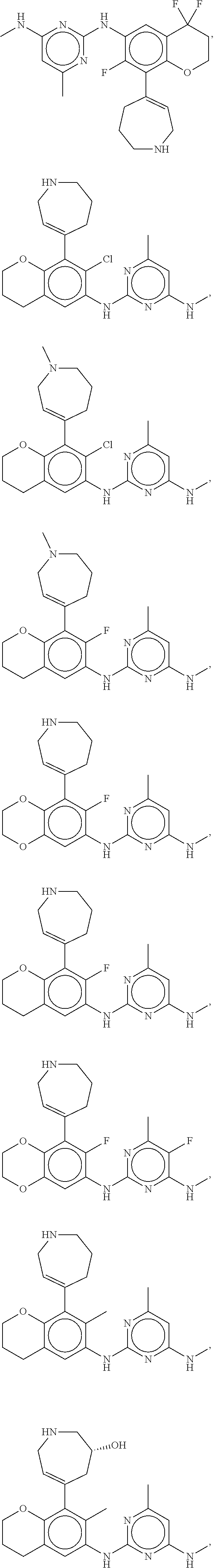

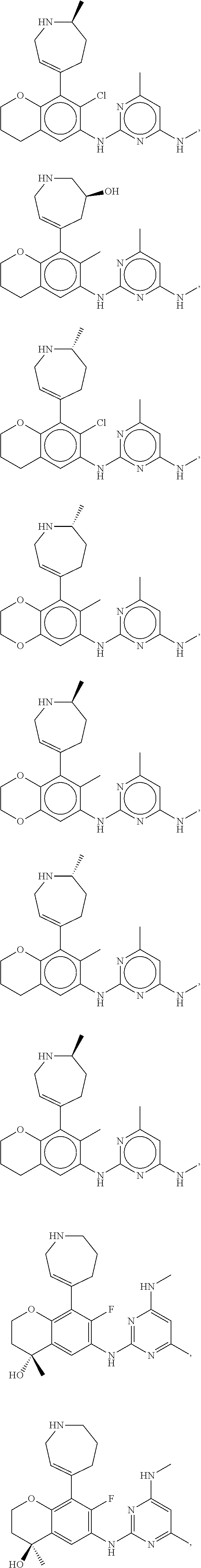

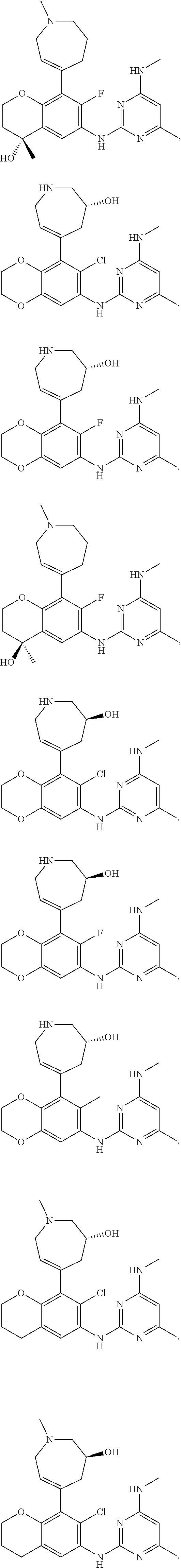

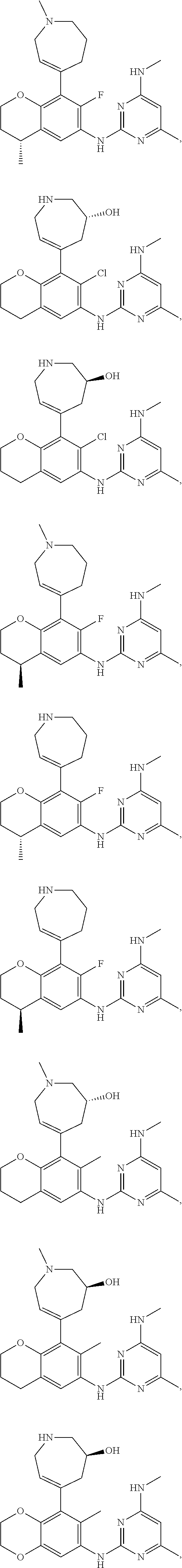

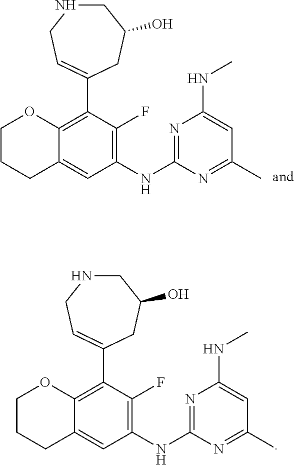

Embodiment 286. The compound of any one of embodiments 1, 4, 5-12, 16-26 and 242-244, or a pharmaceutically acceptable salt thereof, wherein the compound is selected from:

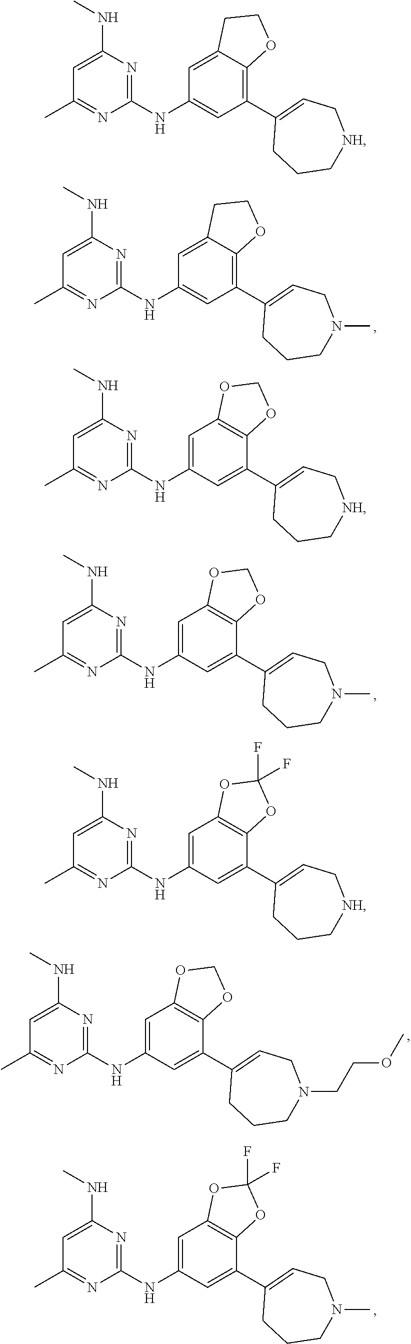

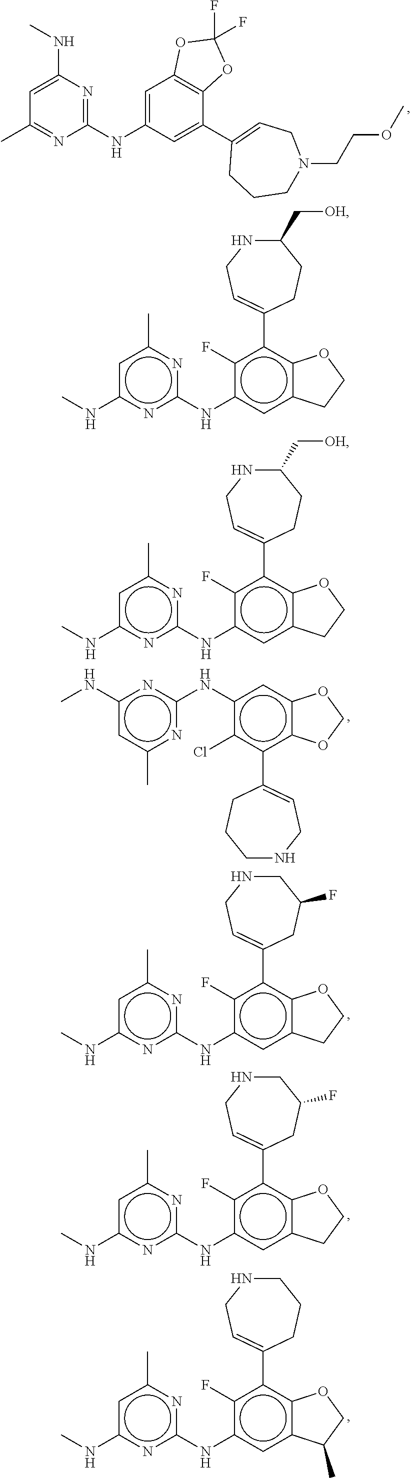

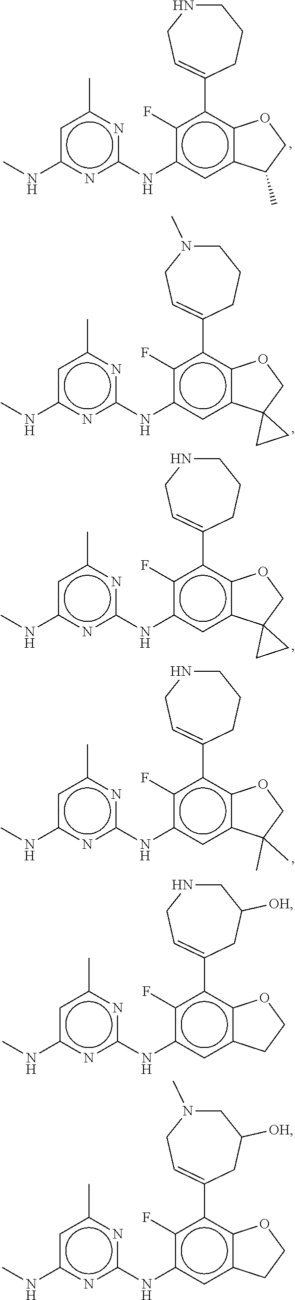

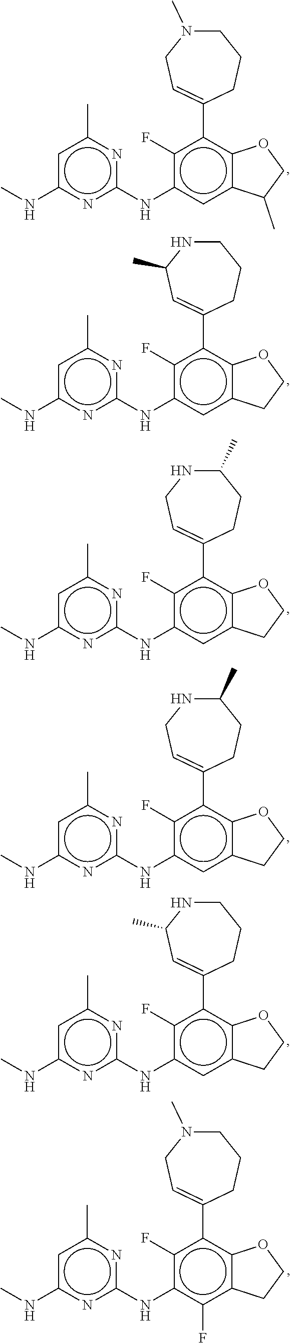

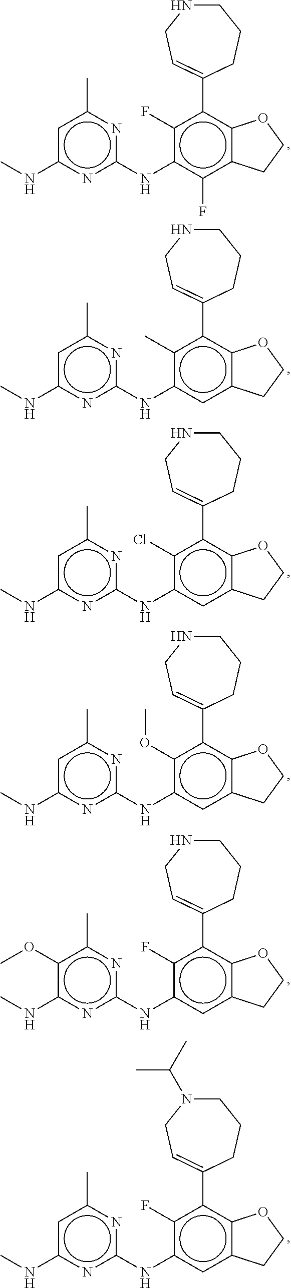

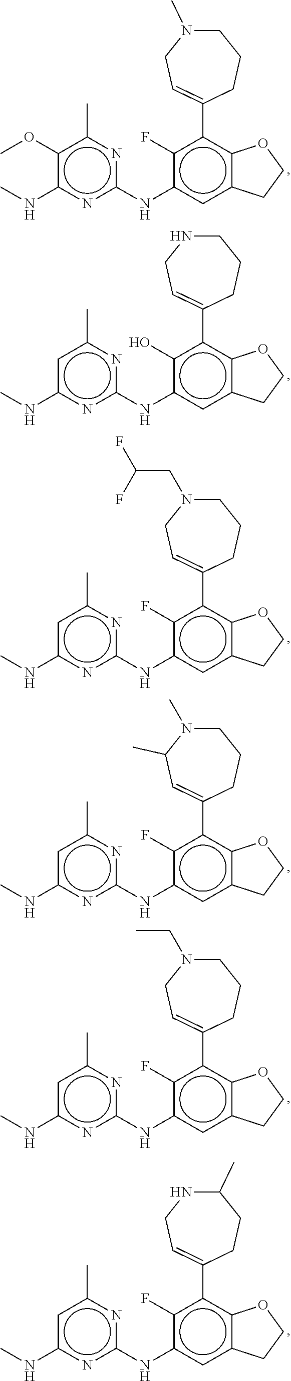

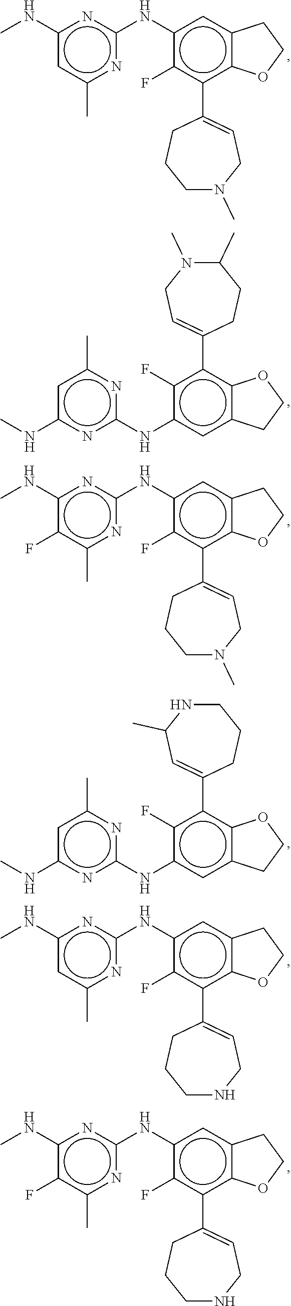

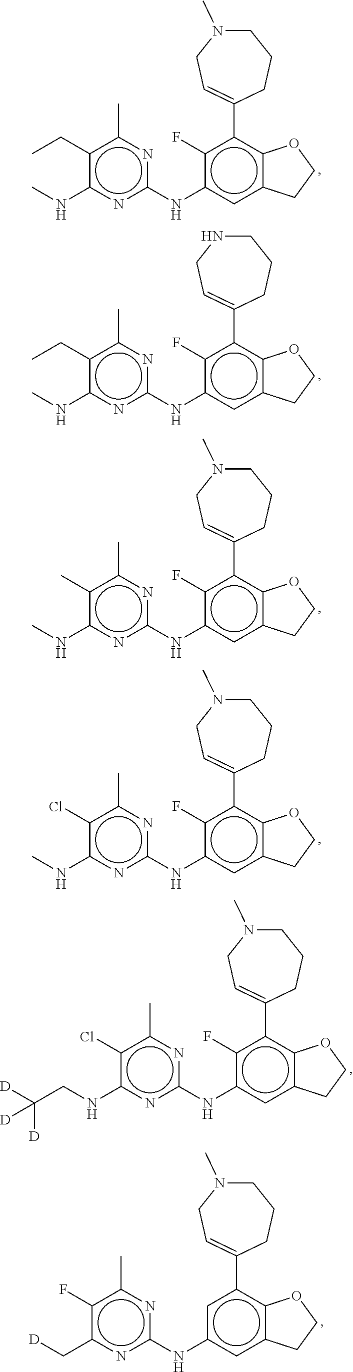

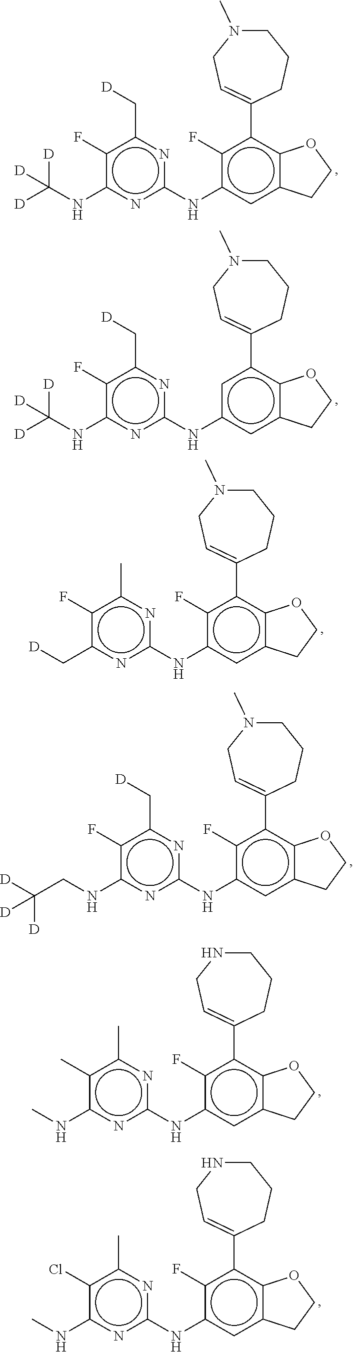

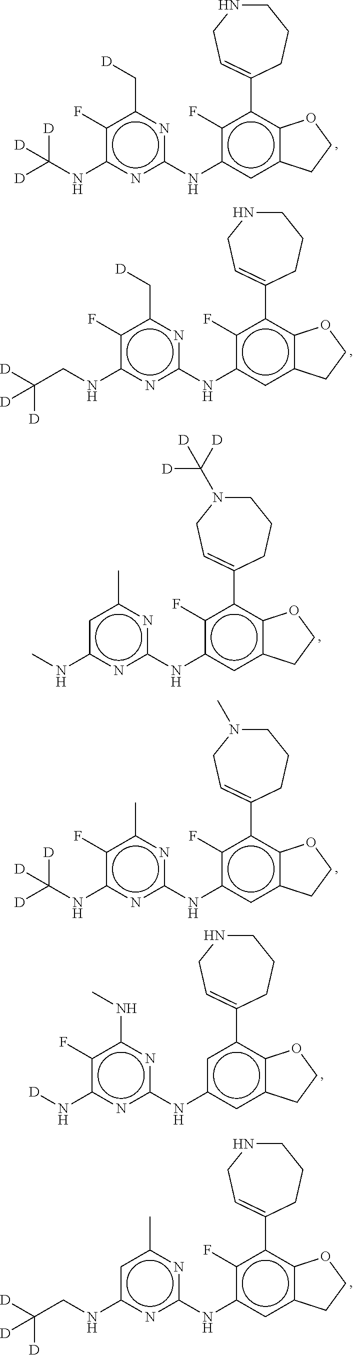

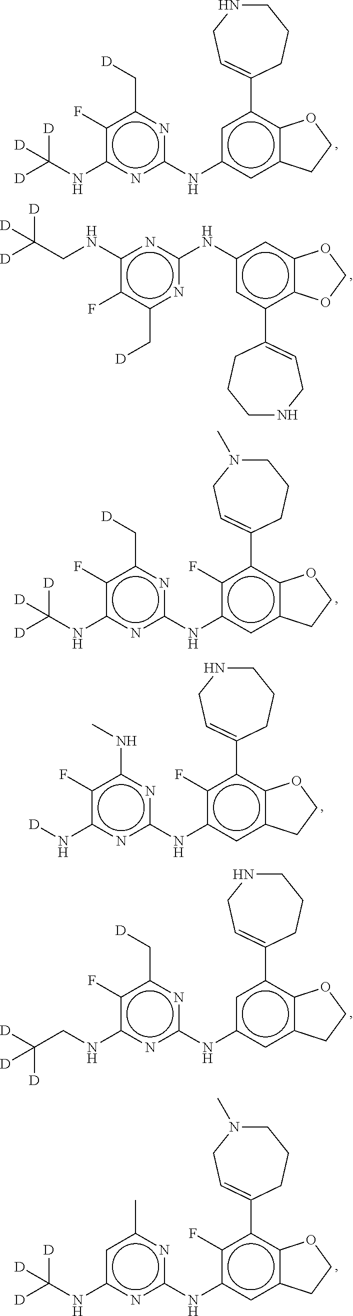

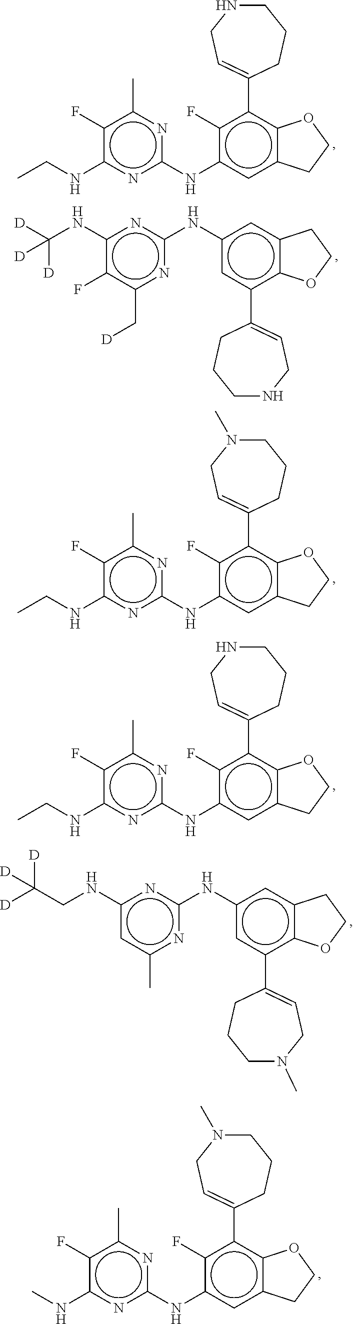

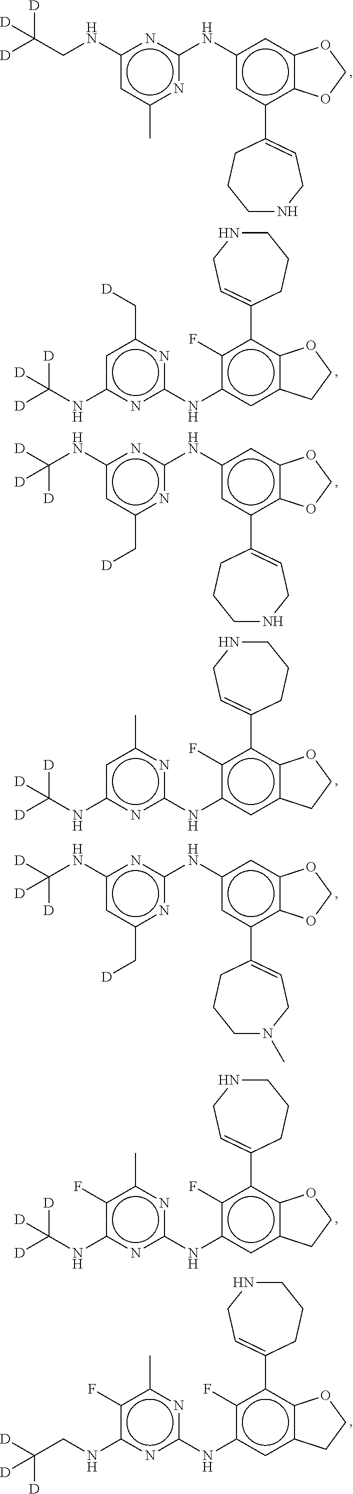

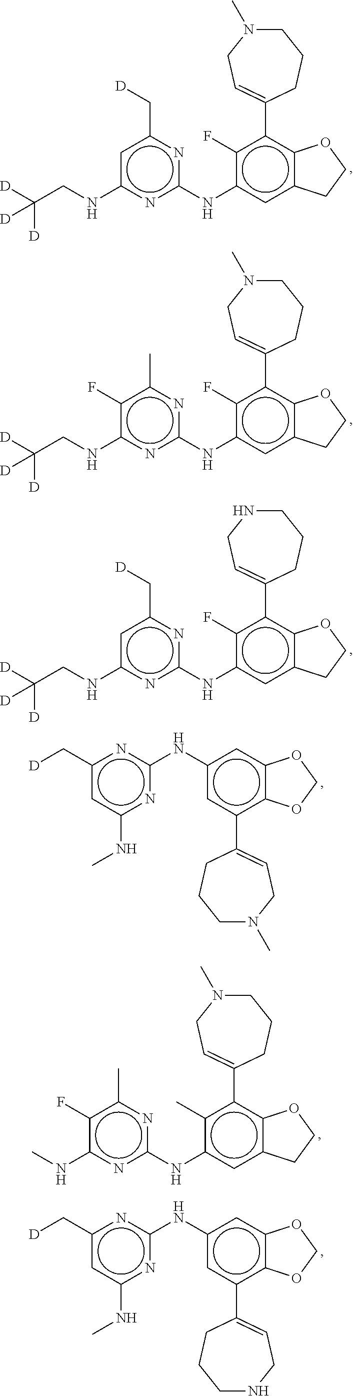

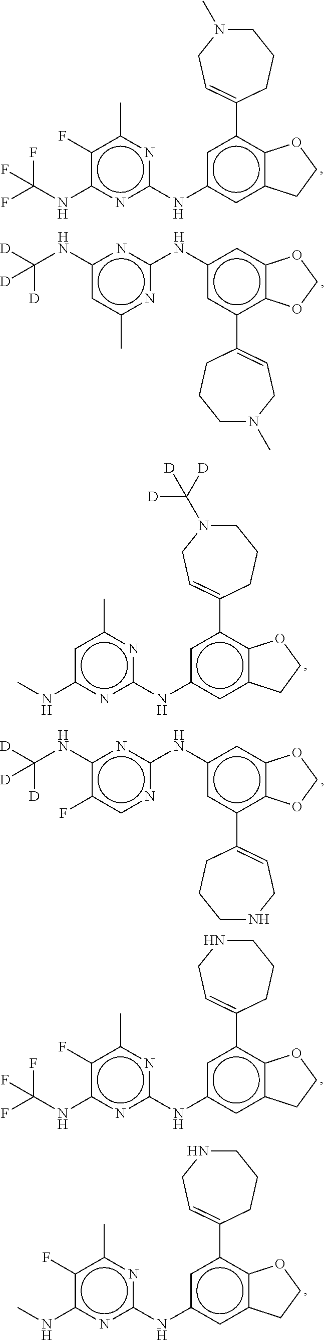

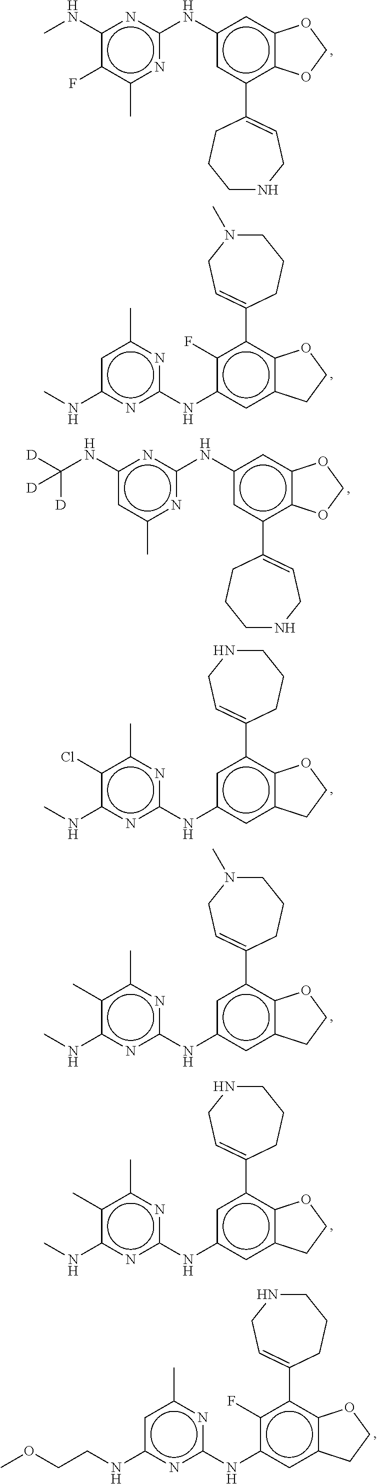

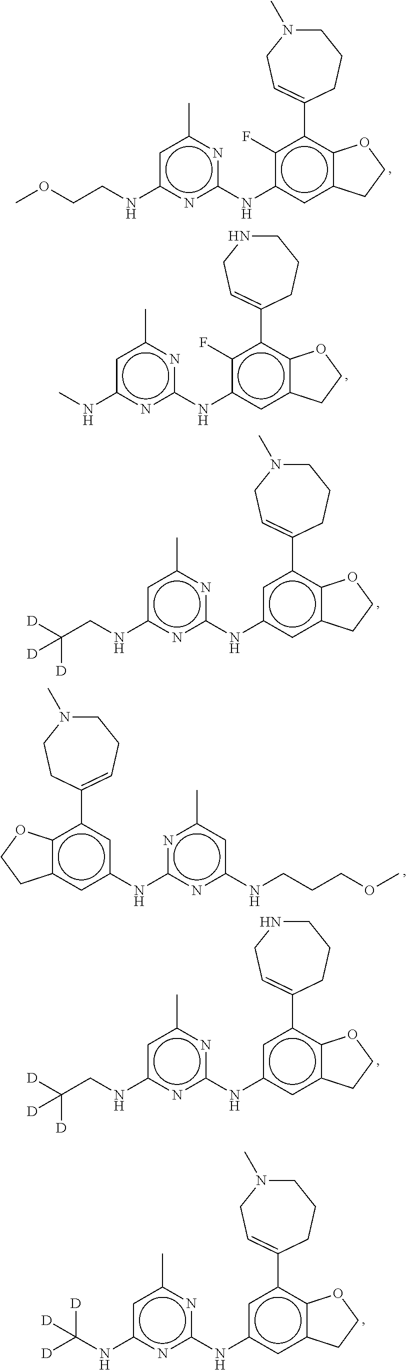

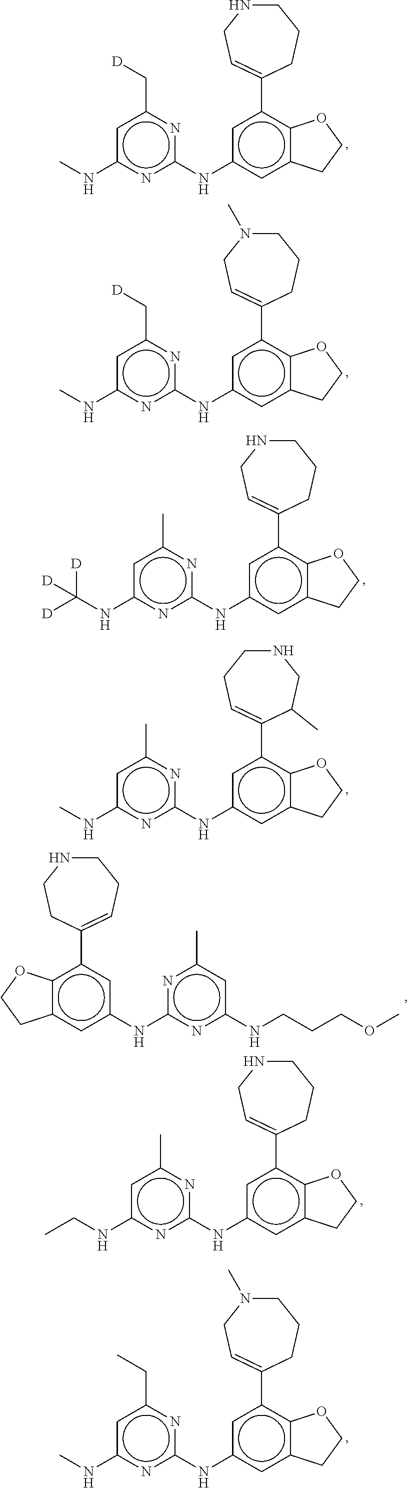

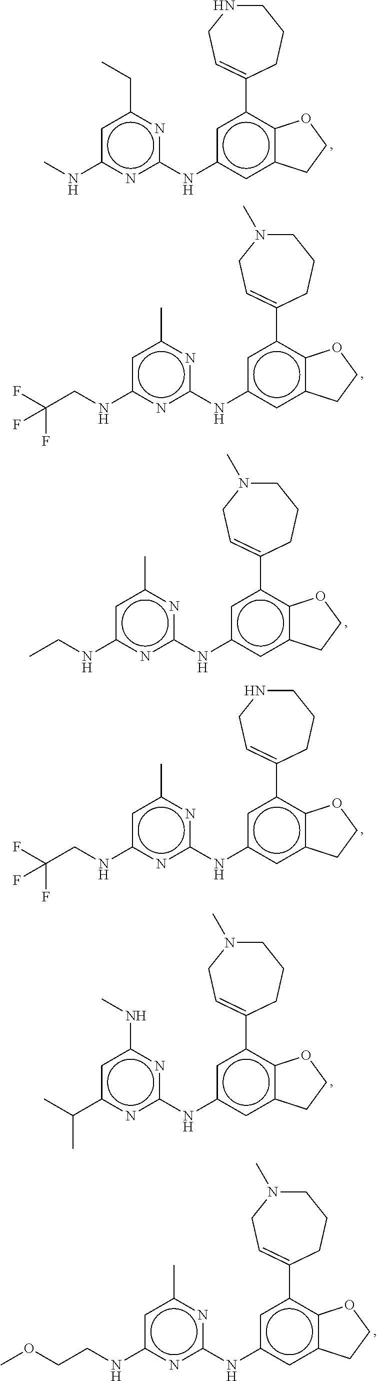

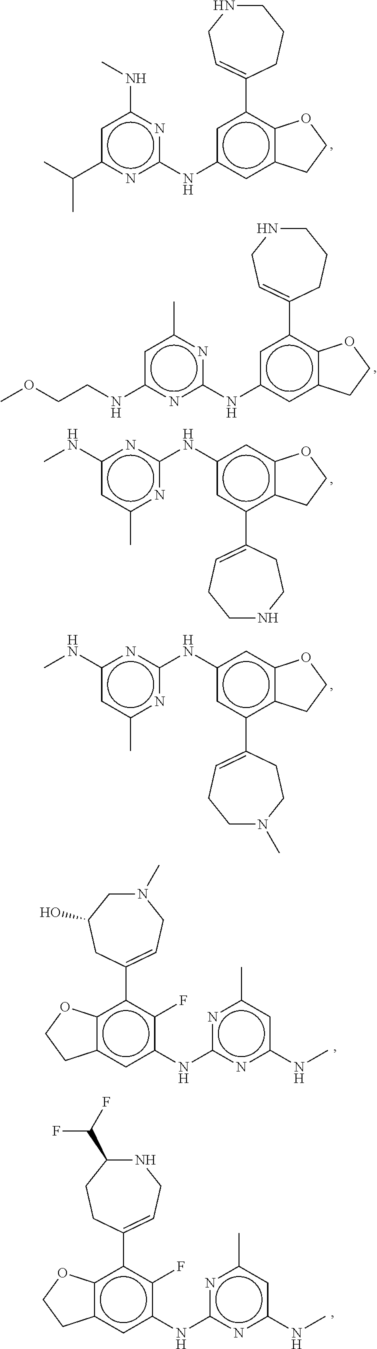

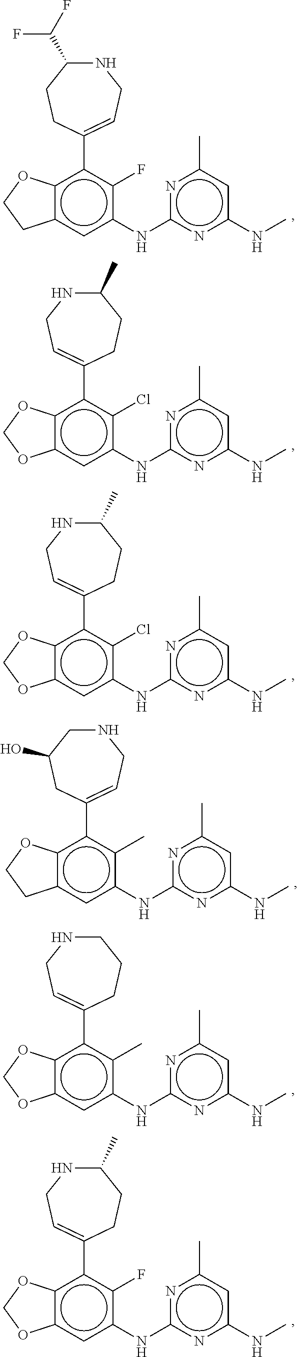

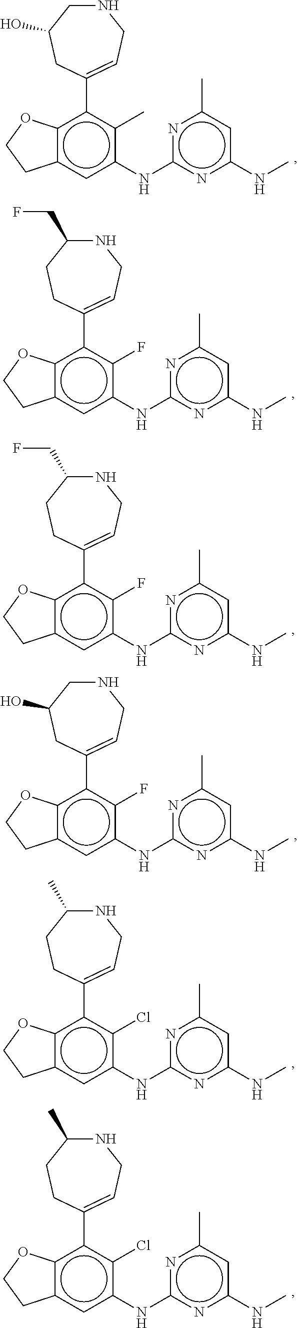

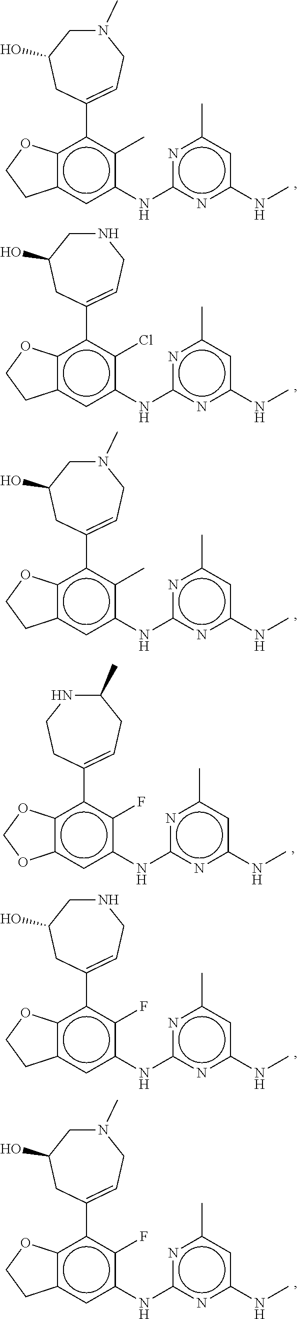

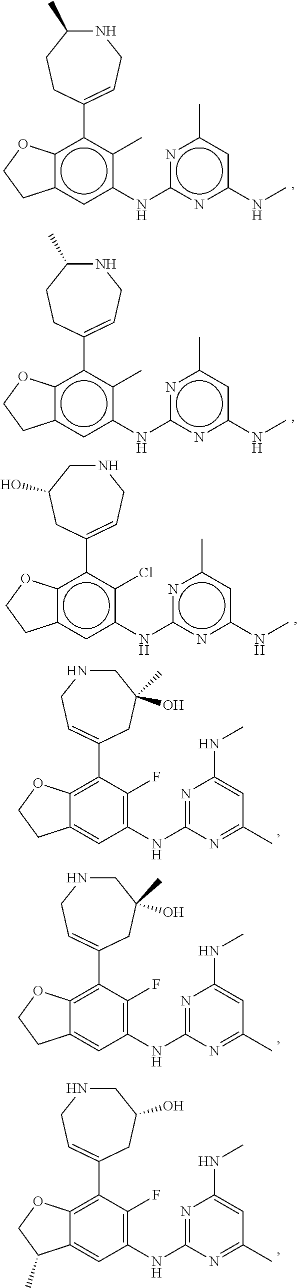

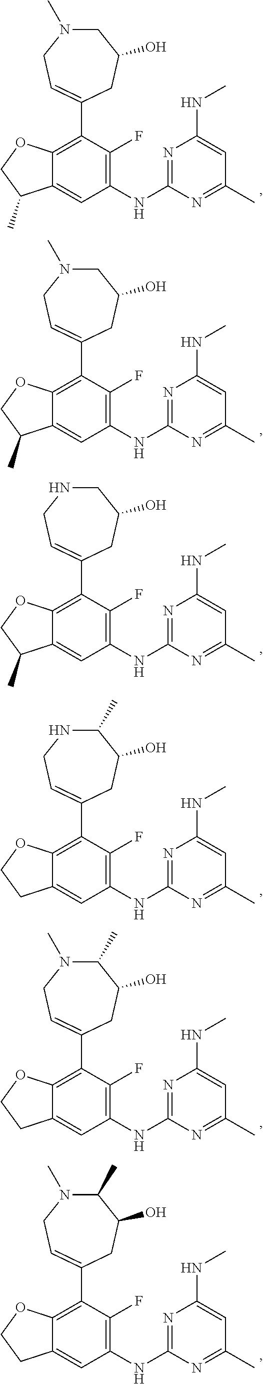

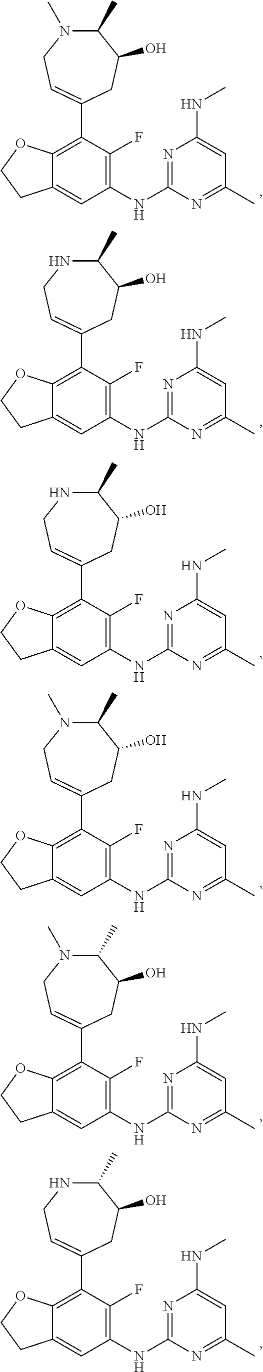

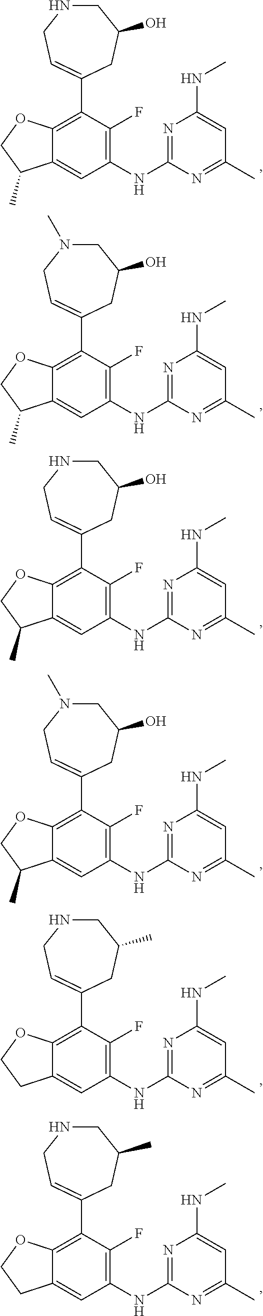

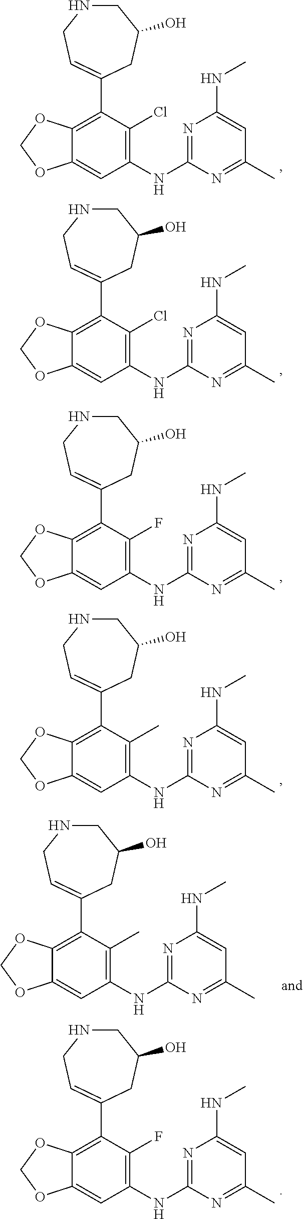

Embodiment 287. The compound of any one of embodiments 1, 4, 5-12, 16-26 and 242-244, or a pharmaceutically acceptable salt thereof, wherein the compound is selected from:

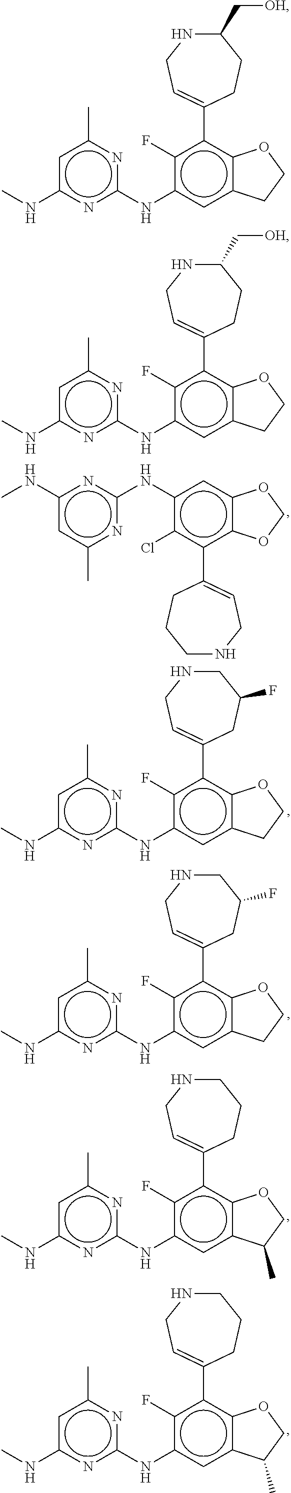

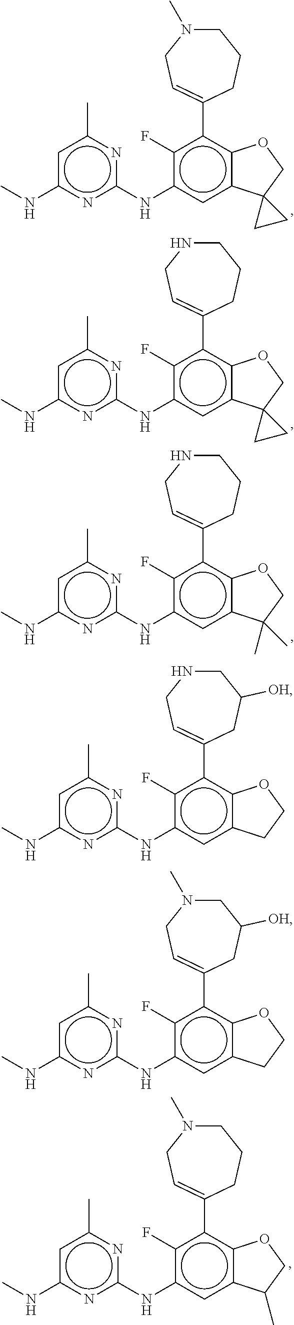

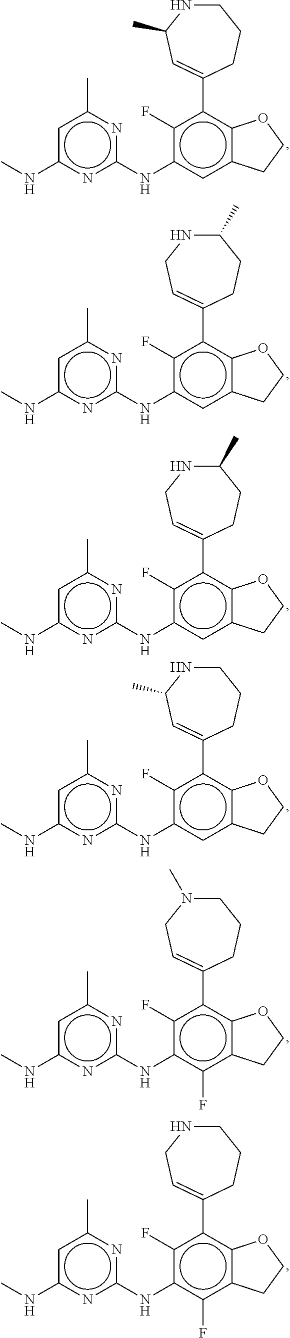

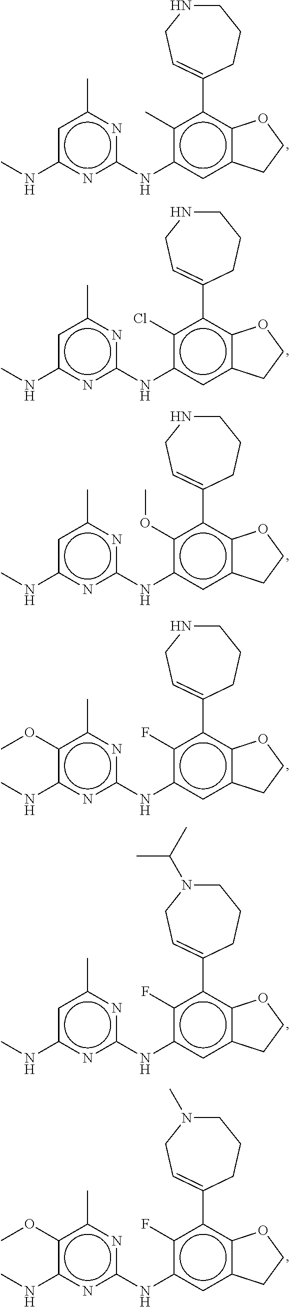

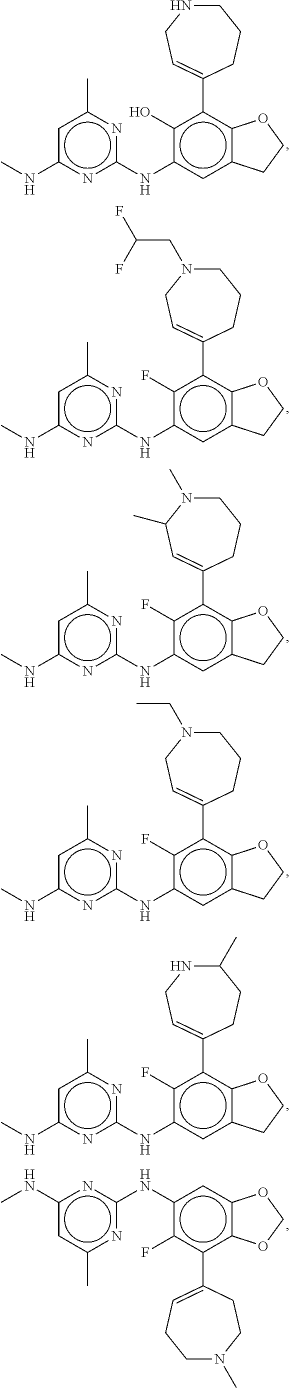

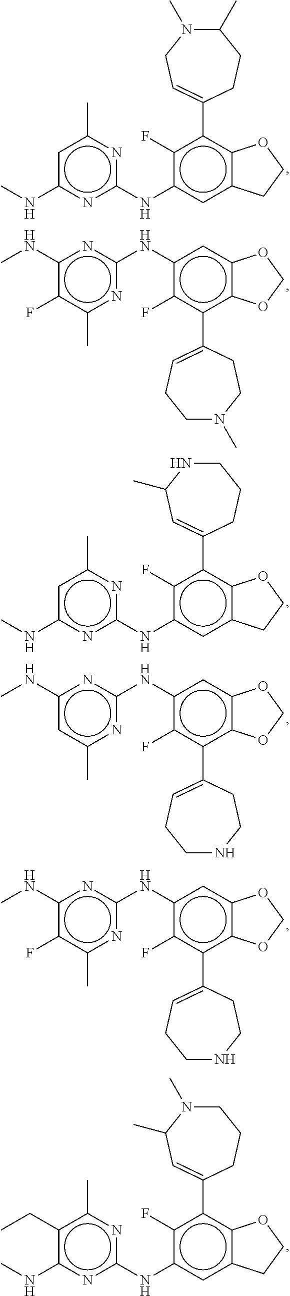

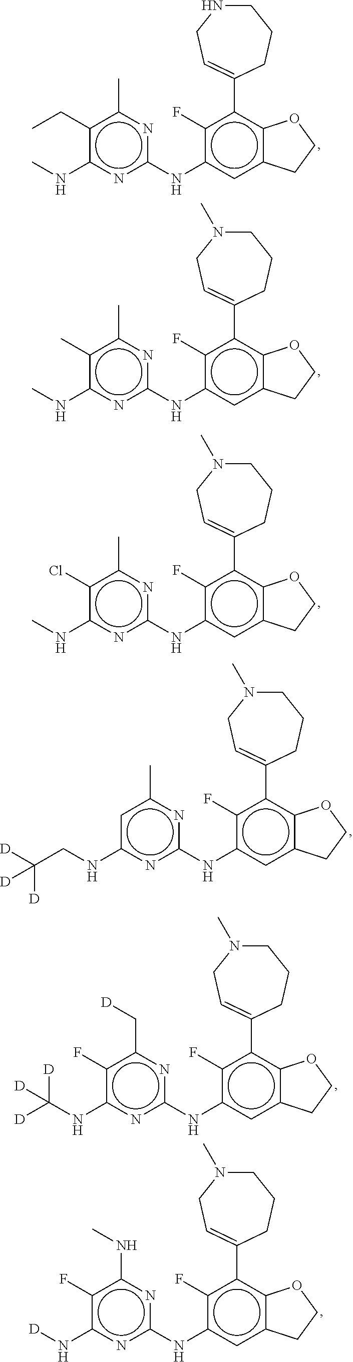

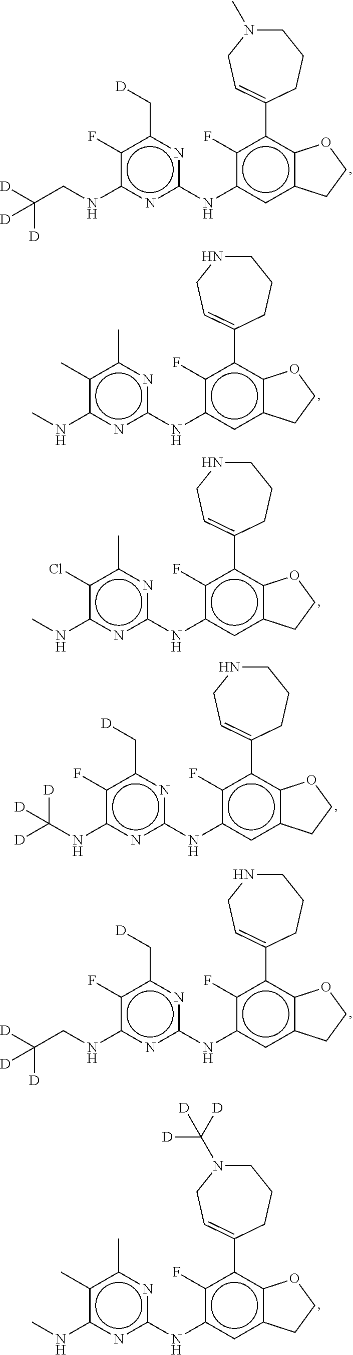

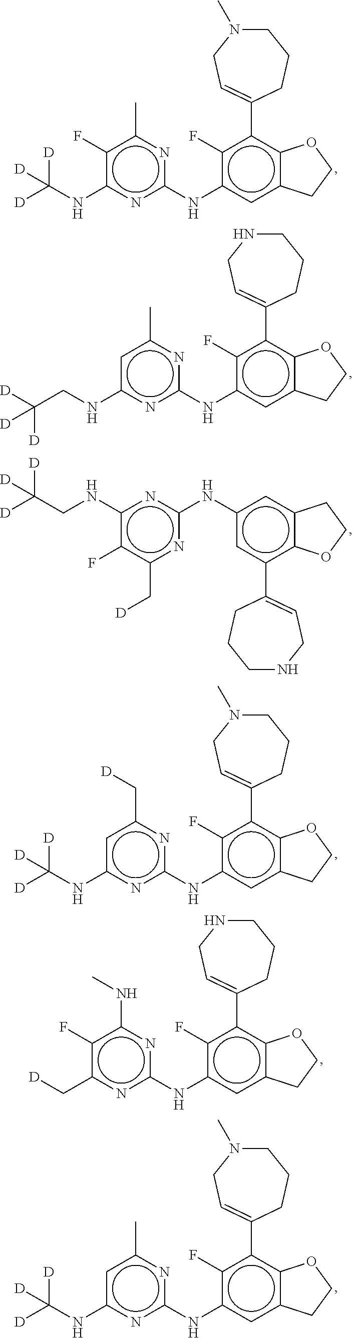

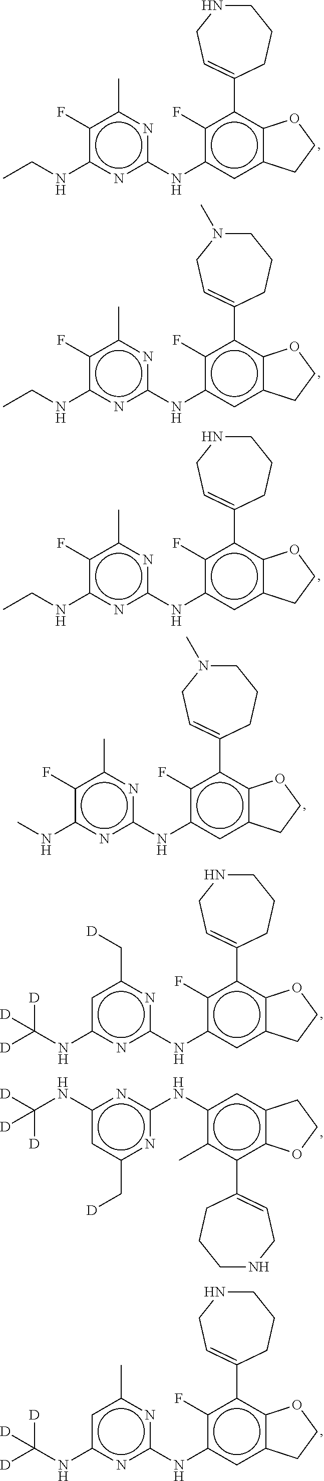

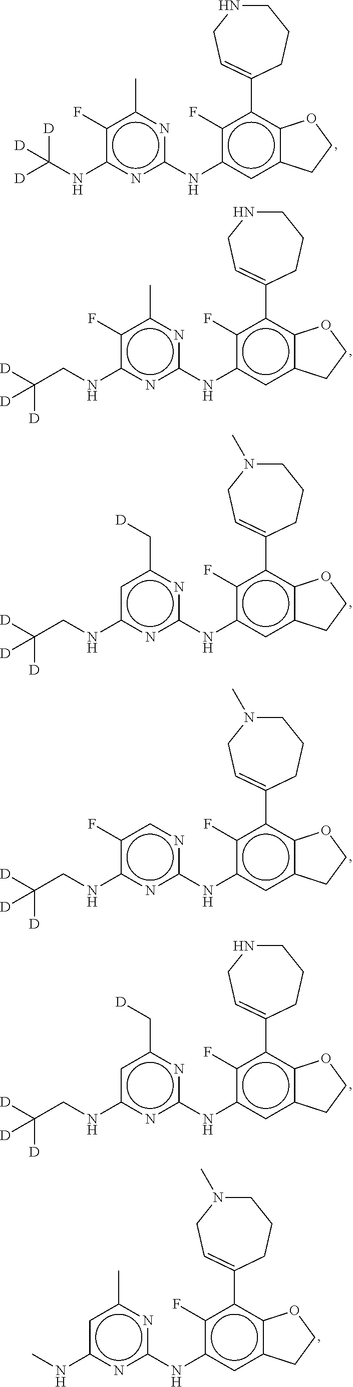

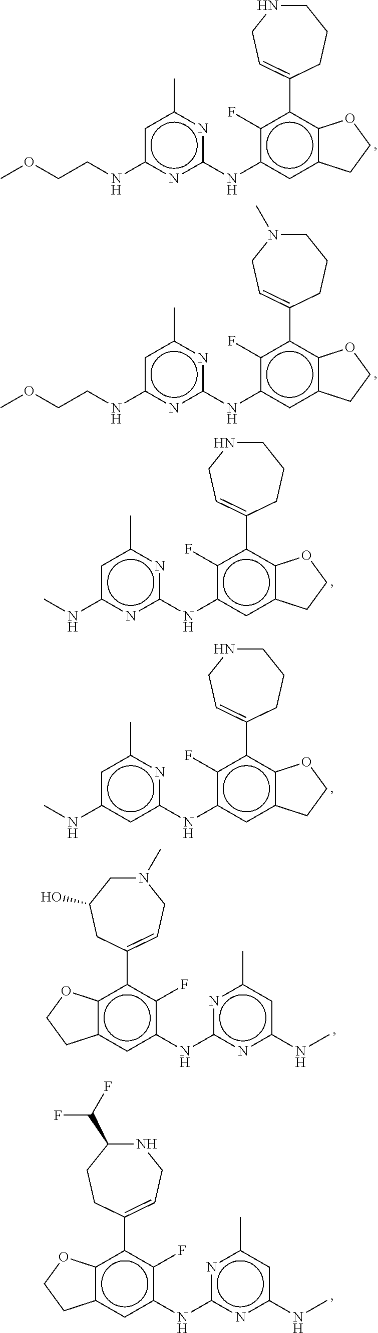

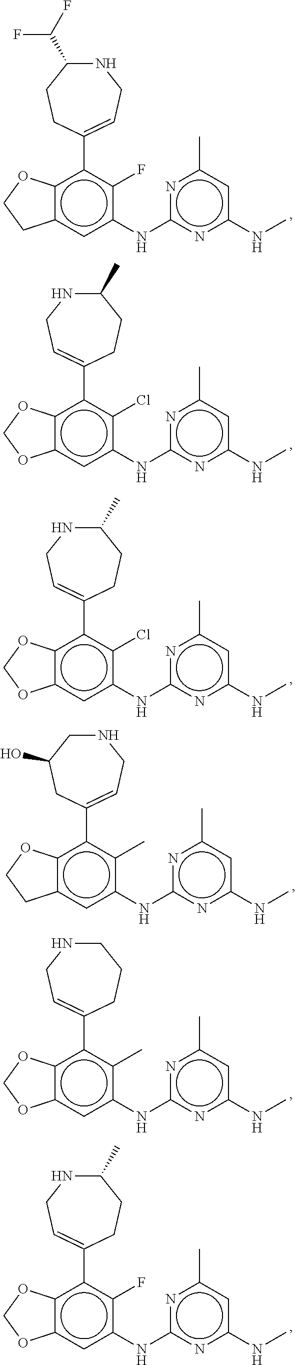

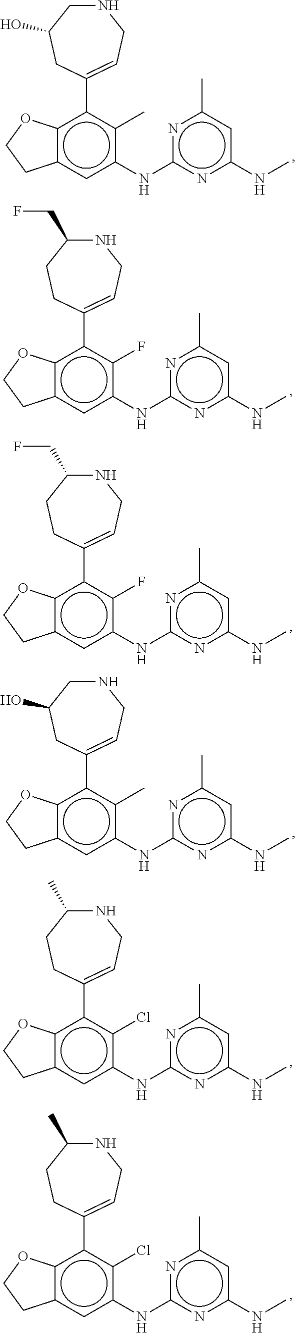

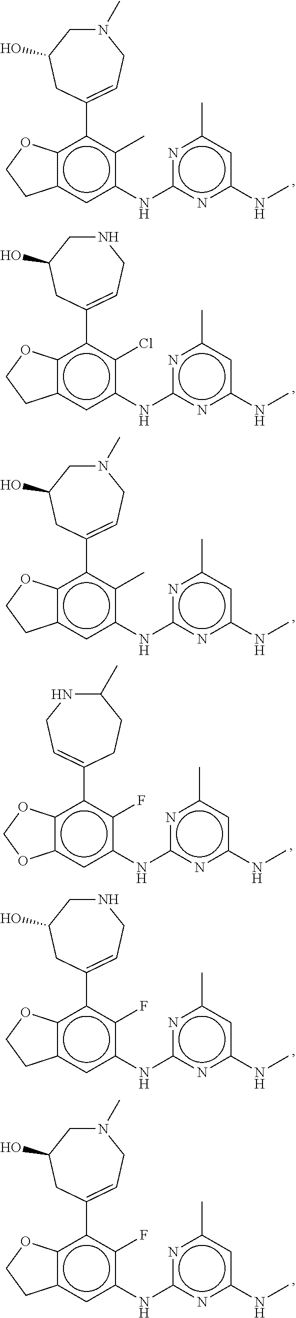

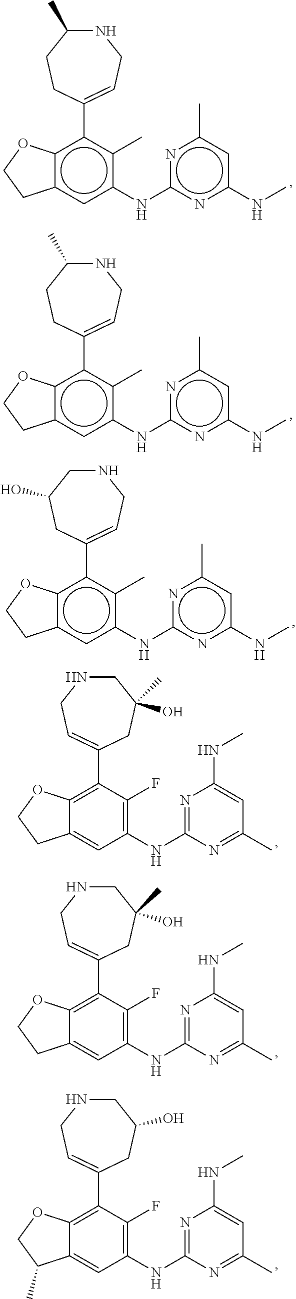

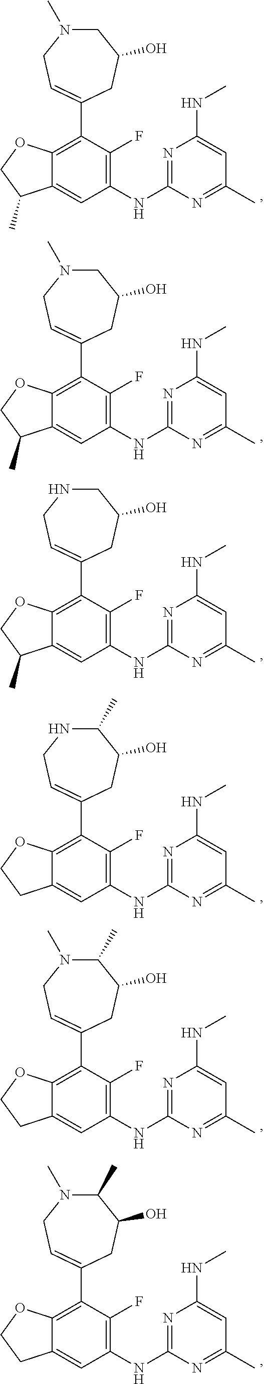

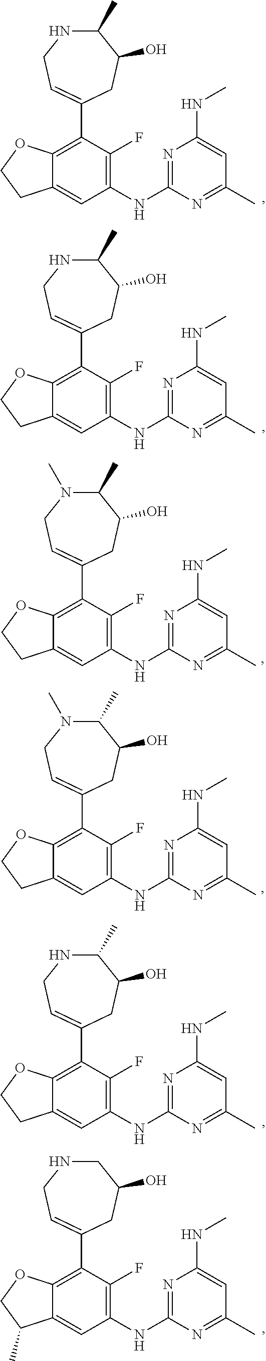

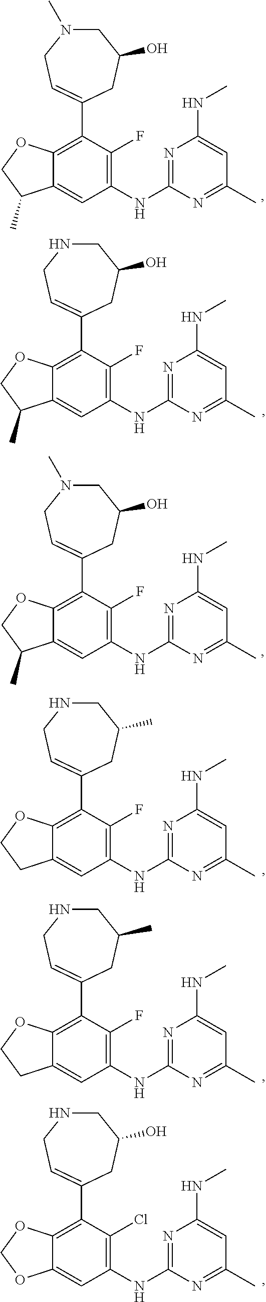

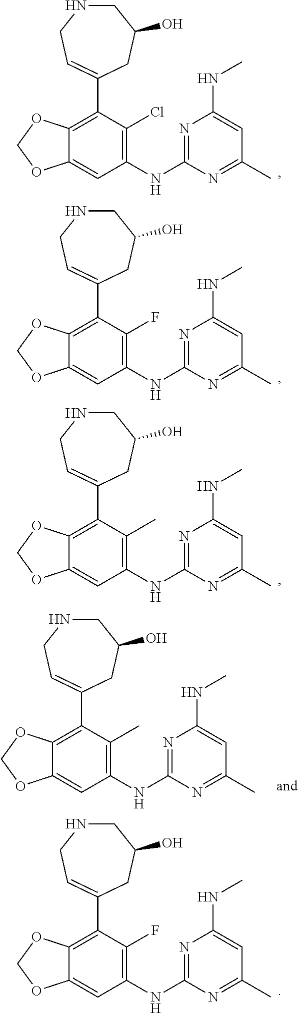

Embodiment 288. A compound of any one of embodiments 1-226, or a pharmaceutically acceptable salt thereof, wherein the compound is selected from the compounds disclosed in the specification and figures, such as a compound of table 1.

Embodiment 289. A composition comprising a compound of any one of embodiments 1-288, or a pharmaceutically acceptable salt thereof, and a pharmaceutically acceptable carrier.

Embodiment 290. A method of treating a disease or disorder that can be treated by modulation of EHMT1 or EHMT2, the method comprising administering to a patient in need thereof a compound of any one of embodiments 1-288, or a pharmaceutically acceptable salt thereof, or a composition of embodiment 289.

Embodiment 291. The method of embodiment 290, wherein the disease or disorder is selected from the group consisting of cancer, sickle cell disease, and beta thalassemia.

Embodiment 292. The method of embodiment 291, wherein the disease or disorder is cancer (e.g., colorectal cancer).

Embodiment 293. The method of embodiment 292, wherein the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

Embodiment 294. The method of embodiment 293, wherein the cancer is selected from the group consisting of a melanoma, bladder cancer, colorectal cancer, head and neck cancer, esophageal cancer, liver cancer, lung cancer, pancreas cancer, and stomach cancer.

Embodiment 295. The method of any one of embodiments 290-294, further comprising administering to the subject at least one additional therapeutic agent.

Embodiment 296. The method of embodiment 295, wherein the at least one additional therapeutic agent is chemotherapy or radiation.

Embodiment 297. Use of a compound of any one of embodiments 1-288, or a pharmaceutically acceptable salt thereof, or a composition of embodiment 289 in the manufacture of a medicament for the treatment of a disease or disorder that can be treated by modulation of EHMT1 or EHMT2.

Embodiment 298. Use of the compound of any one of embodiments 1-288 in the manufacture of a medicament for the treatment of cancer.

Embodiment 299. The use of embodiment 297, wherein the disease or disorder is selected from the group consisting of cancer, sickle cell disease, and beta thalassemia.

Embodiment 300. The use of embodiment 299, wherein the disease or disorder is cancer (e.g., colorectal cancer).

Embodiment 301. The use of embodiment 300, wherein the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

Embodiment 302. The use of embodiment 301, wherein the cancer is selected from the group consisting of a melanoma, bladder cancer, colorectal cancer, head and neck cancer, esophageal cancer, liver cancer, lung cancer, pancreas cancer, and stomach cancer.

Embodiment 303. The use of any one of embodiments 297-302, wherein the medicament is configured for administration with at least one additional therapeutic agent.

Embodiment 304. The use of embodiment 303, wherein the at least one additional therapeutic agent is chemotherapy or radiation.

Embodiment 305. Use of a compound of any one of embodiments 1-288, or a pharmaceutically acceptable salt thereof, or a composition of embodiment 289 for the treatment of a disease or disorder that can be treated by modulation of EHMT1 or EHMT2.

Embodiment 306. The use of embodiment 305, wherein the disease or disorder is selected from the group consisting of cancer, sickle cell disease, and beta thalassemia.

Embodiment 307. The use of embodiment 306, wherein the disease or disorder is cancer (e.g., colorectal cancer).

Embodiment 308. The use of embodiment 307, wherein the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

Embodiment 309. The use of embodiment 308, wherein the cancer is selected from the group consisting of a melanoma, bladder cancer, colorectal cancer, head and neck cancer, esophageal cancer, liver cancer, lung cancer, pancreas cancer, and stomach cancer.

Embodiment 310. The use of any one of embodiments 305-309, wherein the use comprises a combination of compound of any one of embodiments 1-288, or a pharmaceutically acceptable salt thereof, or a composition of embodiment 289 and at least one additional therapeutic agent.

Embodiment 311. The use of embodiment 310, wherein the at least one additional therapeutic agent is chemotherapy or radiation.

Embodiment 312. A compound of any one of embodiments 1-288, or a pharmaceutically acceptable salt thereof, or a composition of embodiment 289 for use in treating a disease or disorder that can be treated by modulation of EHMT1 or EHMT2.

Embodiment 313. The compound for use of embodiment 312, wherein the disease or disorder is selected from the group consisting of cancer, sickle cell disease, and beta thalassemia.

Embodiment 314. The compound for use of embodiment 313, wherein the disease or disorder is cancer (e.g., colorectal cancer).

Embodiment 315. The compound for use of embodiment 314, wherein the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

Embodiment 316. The compound for use of embodiment 315, wherein the cancer is selected from the group consisting of a melanoma, bladder cancer, colorectal cancer, head and neck cancer, esophageal cancer, liver cancer, lung cancer, pancreas cancer, and stomach cancer.

Embodiment 317. The compound for use of any one of embodiments 312-316, wherein the use comprises a combination of compound of any one of embodiments 1-288, or a pharmaceutically acceptable salt thereof, or a composition of embodiment 289 and at least one additional therapeutic agent.

Embodiment 318. The compound for compound for use of embodiment 310, wherein the at least one additional therapeutic agent is chemotherapy or radiation.

### EXAMPLES

Examples are provided below to facilitate a more complete understanding of the invention. The following examples illustrate exemplary modes of making and practicing the invention. However, the scope of the invention is not limited to specific embodiments disclosed in these Examples, which are for purposes of illustration only, since alternative methods can be utilized to obtain similar results.

Compounds in Table 1 have been prepared by the methods described in the Examples below or methods that are similar to those described in the Examples. Compounds in Table 1 that are marked with an asterisk (*) can be prepared by methods similar to those described in the Examples below.

General. All reagents and anhydrous solvents were purchased from commercial vendors and used as received, unless otherwise mentioned. NMR spectra were recorded on a Bruker 400 (400 MHz ¹H, 75 MHz ¹³C) or Varian (400 MHz ¹H, 75 MHz ¹³C) spectrometer. Proton and carbon chemical shifts are reported in ppm (δ) referenced to the NMR solvent. Data are reported as follows: chemical shifts, multiplicity (br=broad, s=singlet, t=triplet, q=quartet, m=multiplet; coupling constant (s) in Hz). Silica gel chromatography was performed on Biotage instruments using pre-packaged disposable SiO₂ stationary phase columns with eluent flow rate range of 15 to 200 mL/min and eluents were detected with using UV (254 and 280 nm). Reverse phase preparative HPLC was carried out using C18 columns, UV detection (215, 220 and 254 nm), and eluting with gradients of MeCN in water (10 mM NH₄HCO₃), MeCN in water (0.04% HCl), or MeCN in water (0.2% HCOOH). Analytical HPLC was performed using a Shimadzu 20AB (Gradient: 10-80% B in 3.00 min, hold at 80% B for 1.0 min, 80-10% B in 0.01 min, and hold at 10% for 0.50 min; Flow rate: 0.5 ml/min during 0.01-4.00 min; 1.0 ml/min during 4.01-4.50 min; Mobile phase A: 0.037% trifluoroacetic acid in water, Mobile phase B: 0.018% trifluoroacetic acid in acetonitrile; Column: Kinetex 5 um C18 100A 50*2.1 mm, diode array (DAD)). Liquid Chromatography/Mass Spectrometry (LCMS) was performed on Agilent 1200 & 61101B (Gradient: 5% B in 0.40 min, 5-95% B at 0.4-3.0 min, hold 95% B for 1.00 min, and 95-5% B in 0.01 min; Flow rate: 1.0 ml/min; Mobile phase A: 0.037% trifluoroacetic acid in water, Mobile phase B: 0.0180% trifluoroacetic acid in acetonitrile; Column: Kinetex C18 50*2.1 mm column (5 um particles), diode array (DAD) and electrospray ionization). Intermediates were analyzed by LCMS using (1) Shimadzu LC-20AD&MS 2020 (Column: Luna-C18 2.0*30 mm (3 um particles), diode array (DAD), positive electrospray ionization for MS; Mobile phase A: 0.037% trifluoroacetic acid in water, Mobile phase B: 0.018% trifluoroacetic acid in acetonitrile; Gradient: 10-80% B in 4.30 min 0.10% B in 0.01 min, 10-80% B (0.01-3.50 min), 80-10% B (3.50-3.80 min), with a hold at 10% B for 0.50 min; Flow rate: 0.8 mL/min (0.01-3.80 min) and 1.2 mL/min (3.81-4.30 min)), or (2) Agilent 1200 & 6110B/Agilent 1200 & 1956A (Column: Xbridge Shield RP18 2.1*50 mm, (5 um particles), diode array (DAD), positive electrospray ionization for MS; Mobile phase A: 10 mM ammonium bicarbonate in water, Mobile phase B: acetonitrile; Gradient: 10-80% B in 3.00 min 0.10% B in 0.00 min, 10-80% B (0.00-2.00 min) with a hold at 80% B for 0.48 min, 80-10% B (2.48-2.50 min) with a hold at 10% B for 0.5 min; Flow rate: 1.0 mL/min (0.00-2.48 min) and 1.2 mL/min (2.50-3.00 min)).

TABLE 2
Abbreviations

| | |
|---|---|
| ACN | acetonitrile |
| AcOH | acetic acid |
| BINAP | 2,2′-bis(diphenylphosphino)- |
| | 1,1′-binaphthyl |
| BrettPhos | [(2-di-cyclohexylphosphino- |
| PdG | 3,6-dimethoxy-2′,4′,6′- |
| | triisopropyl-1,1′-biphenyl)-2- |
| | (2′-amino-1,1′- |
| | biphenyl)]palladium(II) |
| | methanesulfonate |
| Bn | benzyl |
| Boc | t-butoxycarbonyl |
| t-BuOK | potassium tert-butoxide |
| DAD | diode array |
| DCM | dichloromethane |
| DIEA | N,N-diisopropylethylamine |
| DIPEA | N,N-diisopropylethylamine |
| DMF | N,N-dimethylformamide |
| DMSO | dimethyl sulfoxide |
| DPPA | diphenylphosphoryl azide |
| DPBS | Dulbecco′s phosphate buffered |
| | saline |
| dppf | 1,1′- |
| | bis(diphenylphosphino) |
| | ferrocene |
| EHMT1 | euchromatic histone lysine |
| | methyltransferase 1 |
| EHMT2 | euchromatin histone |
| | methyltransferase II |
| EtOAc | ethyl acetate |
| EtOH | ethanol |
| HPLC | high performance liquid |
| | chromatography |
| ³H-SAM | ³H-labeled S- |
| | adenosylmethionine |
| i-PrOH | isopropyl alcohol |
| KOAc | potassium acetate |
| LCMS | liquid chromatography-mass |
| | spectrum |
| LDV | low dead volume |
| Me | methyl |
| MeOH | methanol |
| MeCN | acetonitrile |
| NMR | nuclear magnetic resonance |
| | spectroscopy |
| PBS | phosphate-buffered saline |
| Pd₂(dba)₃ | tris(dibenzylideneacetone) |
| | dipalladium |
| Pd(dppf) | [1,1′- |
| Cl₂ | bis(diphenylphosphino) |
| | ferrocene]dichloropalladium(II) |
| PPA | phenylpropanolamine |
| PSI | pounds per square inch |
| Py or pyr | pyridine |
| SAH | S-adenosyihomocysteine |
| SAM | S-adenosylmethionine |
| SPA | scintillation proximity assay |
| TCEP | tris (2-carboxyethyl)phosphine |
| TEA | triethylamine |
| TFA | trifluoroacetic acid |
| THF | tetrahydrofuran |
| Tol | toluene |
| TLC | thin layer chromatography |
| UV | tltraviolet-visible |

### EXAMPLE 1. Synthesis of Compound 102

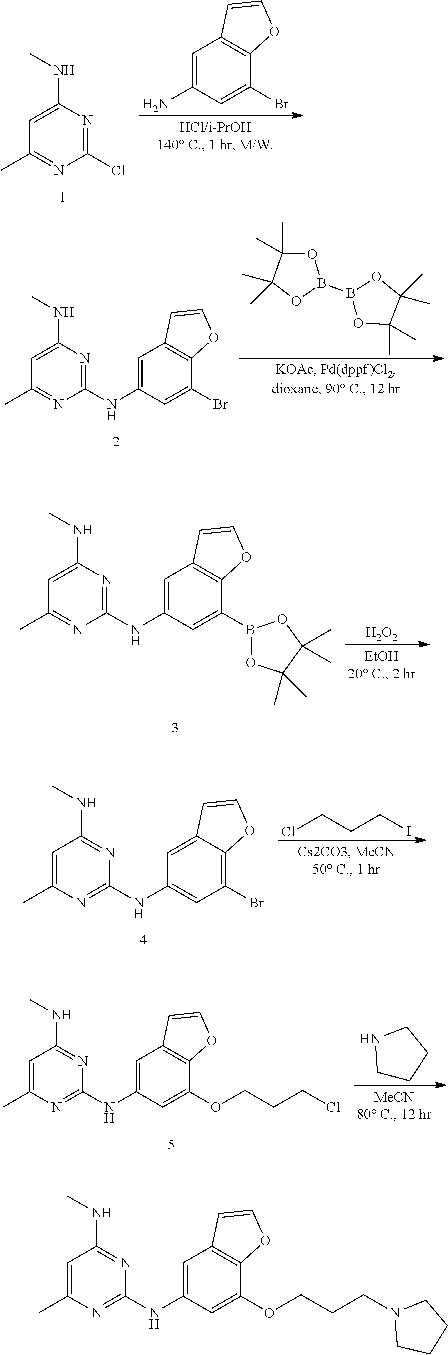

### Step 1. N2-(7-bromobenzofuran-5-yl)-N4,6-dimethyl-pyrimidine-2, 4-diamine

To the mixture of 2-chloro-N,6-dimethyl-pyrimidin-4-amine (200 mg, 1.27 mmol) and 7-bromobenzofuran-5-amine (269.09 mg, 1.27 mmol) in i-PrOH (2 mL) was added HCl (4.63 mg, 126.90 umol, 5.78 uL), then the mixture was stirred at 140° C. in the microwave for 1 hrs. The reaction was filtered and concentrated under reduced pressure to give N2-(7-bromobenzofuran-5-yl)-N4,6-dimethyl-pyrimidine-2,4-diamine (400 mg, crude) as a taupe solid.

### Step 2. [5-[[4-methyl-6-(methylamino)pyrimidin-2-yl]amino]benzofuran-7-yl]-boronic acid

To a mixture of N2-(7-bromobenzofuran-5-yl)-N4,6-dimethyl-pyrimidine-2,4-diamine (1.3 g, 3.90 mmol) and 4,4,5,5-tetramethyl-2-(4,4,5,5-tetramethyl-1,3,2-dioxaborolan-2-yl)-1,3,2-dioxaborolane (1.98 g, 7.80 mmol) in dioxane (20 mL) was added potassium acetate (1.15 g, 11.71 mmol, 731.71 uL) and cyclopentyl(diphenyl)phosphane dichloropalladium iron (570.99 mg, 780.35 umol), then the mixture was stirred at 90° C. for 12 hrs under N2. LCMS showed the reaction was complete and the desired ms was detected. The reaction was concentrated under reduced pressure to give a residue. The residue was purified by column chromatography (SiO₂, Petroleum ether/Ethyl acetate=1/0 to Ethyl acetate/Methyl alcohol=10/1) to give [5-[[4-methyl-6-(methylamino)pyrimidin-2-yl]amino]benzofuran-7-yl]boronic acid (1 g, crude) as brown solid.

### Step 3. 5-[[4-methyl-6-(methylamino)pyrimidin-2-yl]amino]benzofuran-7-ol

To a mixture of [5-[[4-methyl-6-(methylamino)pyrimidin-2-yl]amino]benzofuran-7-yl]boronic acid (200 mg, 670.91 umol) in EtOH (5 mL) was added H₂O₂ (190 mg, 1.68 mmol, 30% purity), then the mixture was stirred at 20° C. for 2 h. LCMS showed the starting material remained and the desired ms was detected. The reaction was added saturated sodium sulfite solution (5 mL) stirred at 20° C. for 10 min, then was added water (10 mL), then extracted with ethyl acetate (3*10 mL) and dried over anhydrous sodium sulfate, filtered and concentrated under reduced pressure to give a residue. The residue was purified by prep-TLC (SiO₂, Ethyl acetate/methyl alcohol=5:1) to give 5-[[4-methyl-6-(methylamino)pyrimidin-2-yl]amino]benzofuran-7-ol (20 mg, crude) as a white solid.

### Step 4. N2-[7-(3-chloropropoxy)benzofuran-5-yl]-N4,6-dimethyl-pyrimidine-2, 4-diamine

To the mixture of 5-[[4-methyl-6-(methylamino)pyrimidin-2-yl]amino]benzofuran-7-ol (20 mg, 74.00 umol) and 1-chloro-3-iodo-propane (15.13 mg, 74.00 umol, 7.96 uL) in CH₃CN (1 mL) was added dicesium carbonate (48.22 mg, 147.99 umol), then the mixture was stirred at 50° C. for 1 hrs. LCMS showed the reaction was complete and the desired ms was detected. The mixture was used for the next step directly.

### Step 5. N4,6-dimethyl-N2-[7-(3-pyrrolidin-1-ylpropoxy)benzofuran-5-yl]pyrimidine-2,4-diamine

To the mixture of N2-[7-(3-chloropropoxy)benzofuran-5-yl]-N4,6-dimethyl-pyrimidine-2,4-diamine (25 mg, 72.09 μmol) and pyrrolidine (25.63 mg, 360.43 μmol, 29.95 μL) in CH₃CN (2 mL), then the mixture was stirred at 80° C. for 12 hrs. LCMS showed the reaction was complete and the desired ms was detected. The reaction was concentrated under reduced pressure to give a residue. The crude product was purified by prep-HPLC(TFA condition: Phenomenex Luna 80*30 mm*3 um; mobile phase: water(0.1% TFA)-ACN; B %: 15%-37%, 8 min) to give N4,6-dimethyl-N2-[7-(3-pyrrolidin-1-ylpropoxy)benzofuran-5-yl]pyrimidine-2,4-diamine (5.7 mg, 11.53 μmol, 15.99% yield, TFA) as yellow solid.

¹H NMR (400 MHz, METHANOL-d₄) δ=7.82-7.77 (m, 1H), 7.55 (s, 1H), 7.14-7.09 (m, 1H), 6.85 (br s, 1H), 6.00-5.96 (m, 1H), 4.39-4.32 (m, 2H), 3.77 (br s, 2H), 3.52-3.47 (m, 2H), 3.21-3.12 (m, 2H), 2.96 (s, 3H), 2.40-2.32 (m, 1H), 2.31-2.27 (m, 3H), 2.24-2.15 (m, 2H), 2.12-2.03 (m, 2H), 2.03-1.97 (m, 1H). MS (ESI): m/z=382.1 [M+H]

### EXAMPLE 2. Synthesis of Compound 103

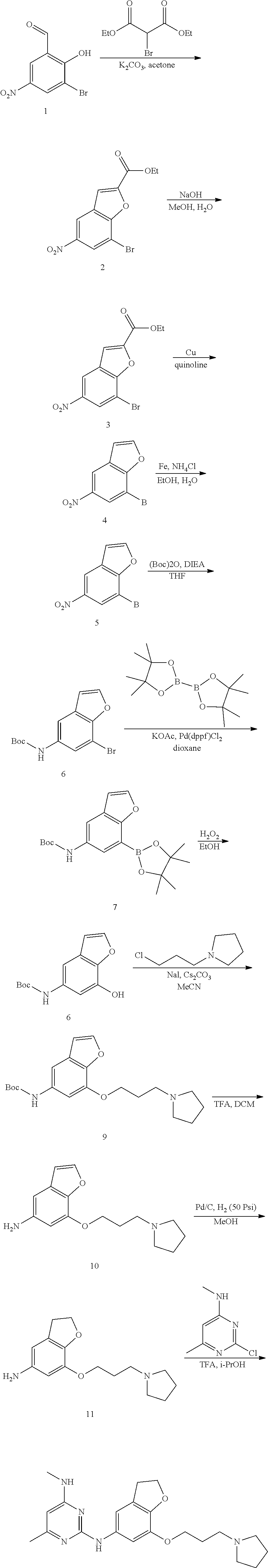

Step 1. Ethyl 7-bromo-5-nitro-benzofuran-2-carboxylate

To a solution of 3-bromo-2-hydroxy-5-nitro-benzaldehyde (15 g, 60.97 mmol) in acetone (250 mL) was added diethyl 2-bromopropanedioate (17.49 g, 73.17 mmol, 12.49 mL) and tripotassium carbonate (16.85 g, 121.94 mmol, 7.36 mL). The mixture was stirred at 60° C. for 12 h. TLC (Petroleum ether: Ethyl acetate=3:1, Rf=0.6) showed the reaction was complete.

The reaction mixture was added to water 300 mL. The mixture was filtered and the filter cake was dried in vacuum to give ethyl 7-bromo-5-nitro-benzofuran-2-carboxylate (20 g, crude) was obtained as a yellow solid.

### Step 2. 7-bromo-5-nitro-benzofuran-2-carboxylic acid

To a mixture of ethyl 7-bromo-5-nitro-benzofuran-2-carboxylate (40 g, 127.35 mmol) in MeOH (20 mL) and H₂O (10 mL) was added Sodium hydroxide (10.19 g, 254.71 mmol, 4.78 mL) at 20° C., then the mixture was stirred at 20° C. for 12 hrs. TLC (Petroleum ether: Ethyl acetate=3:1, Rf=0) showed the reaction was complete. The reaction mixture was concentrated under reduced pressure to give a residue, then adjust pH to about 1. The mixture was filtered and the filter cake was dried in vacuum to give 7-bromo-5-nitro-benzofuran-2-carboxylic acid (30 g, crude) was obtained as a yellow solid.

### Step 3. 7-bromo-5-nitro-benzofuran

To a mixture of 7-bromo-5-nitro-benzofuran-2-carboxylic acid (5 g, 17.48 mmol) in quinoline (50 mL) was added Cu (2.22 g, 34.96 mmol) at 20° C., then the mixture was stirred at 200° C. for 2 h. TLC (petroleum ether: ethyl acetate=3:1, Rf=0.7) indicated starting material was consumed completely, and three major new spots were detected. Two reactions were combined. To the combined mixture was added 12 M HCl (300 mL) stirred at 20° C. for 15 min, then extracted with ethyl acetate (3×200 mL). The combined organic layers were washed with brine (3×200 mL) and dried over anhydrous sodium sulfate, filtered and concentrated under reduced pressure to give 7-bromo-5-nitro-benzofuran (7.5 g, crude) as a brown oil.

### Step 4. 7-bromobenzofuran-5-amine

To a solution of 7-bromo-5-nitro-benzofuran (7.5 g, 30.99 mmol), NH₄Cl (16.58 g, 309.88 mmol) and H₂O (8 mL) in EtOH (80 mL) was added Fe (8.65 g, 154.94 mmol). It was stirred at 100° C. for 12 h. TLC (petroleum ether: ethyl acetate=3:1, Rf=0.5) indicated starting material was consumed completely, and two major new spots were detected. LCMS showed mass of the desired compound. The reaction was filtered and concentrated under reduced pressure, then added water (200 mL), then extracted with ethyl acetate (3×200 mL) and dried over anhydrous sodium sulfate, filtered and concentrated under reduced pressure. The residue was purified by flash chromatography (silica gel, petroleum ether/ethyl acetate=1/0 to 1/1) to give 7-bromobenzofuran-5-amine (5.5 g, 25.94 mmol, 83.70% yield) as an orange oil.

### Step 5. tert-butyl N-(7-bromobenzofuran-5-yl)carbamate

To a solution of 7-bromobenzofuran-5-amine (7 g, 33.01 mmol) and DIPEA (8.53 g, 66.02 mmol, 11.50 mL) in THF (70 mL) was added tert-butoxycarbonyl tert-butyl carbonate (8.65 g, 39.61 mmol, 9.09 mL). It was stirred at 20° C. for 12 h. TLC (petroleum ether: ethyl acetate=3:1, Rf=0.8) indicated 7-bromobenzofuran-5-amine remained, and three major new spots were detected. LCMS showed starting material remained and mass of the desired compound. The reaction was concentrated under reduced pressure to give a residue. The residue was purified by flash chromatography (silica gel, petroleum ether/ethyl acetate=1/0 to 8/1) to give tert-butyl N-(7-bromobenzofuran-5-yl)carbamate (8.45 g, 27.07 mmol, 82.00% yield) as a light-yellow solid.

¹H NMR (400 MHz, METHANOL-d₄) δ ppm 1.53 (s, 9H) 6.86 (d, J=2.25 Hz, 1H) 7.55 (d, J=1.63 Hz, 1H) 7.65 (s, 1H) 7.79 (d, J=2.13 Hz, 1H)

### Step 6. tert-butyl N-[7-(4,4,5,5-tetramethyl-1,3,2-dioxaborolan-2-yl)benzofuran-5-yl]carbamate

To a solution of tert-butyl N-(7-bromobenzofuran-5-yl)carbamate (6.45 g, 20.66 mmol), 4,4,5,5-tetramethyl-2-(4,4,5,5-tetramethyl-1,3,2-dioxaborolan-2-yl)-1,3,2-dioxaborolane (6.30 g, 24.80 mmol) and CH₃COOK (4.06 g, 41.33 mmol) in dioxane (58.71 mL) was added cyclopentyl(diphenyl)phosphane dichloropalladium; iron (1.51 g, 2.07 mmol) under an atmosphere of nitrogen. It was stirred at 90° C. for 12 h under an atmosphere of nitrogen. TLC (petroleum ether: ethyl acetate=5:1, Rf=0.4) indicated starting material was consumed completely, and two major new spots were detected. LCMS showed starting material was consumed completely and mass of the desired compound. The reaction was filtered and concentrated under reduced pressure to give a residue. The residue was purified by flash chromatography (silica gel, petroleum ether/ethyl acetate=1/0 to 5/1) to give tert-butyl N-[7-(4,4,5,5-tetramethyl-1,3,2-dioxaborolan-2-yl)benzofuran-5-yl]carbamate (6.26 g, 17.43 mmol, 84.34% yield) as a light-yellow solid.

¹H NMR (400 MHz, METHANOL-d₄) δ ppm 1.39 (s, 12H) 1.53 (s, 9H) 6.78 (d, J=2.25 Hz, 1H) 7.60 (d, J=2.38 Hz, 1H) 7.74 (d, J=2.25 Hz, 1H) 7.82 (br s, 1H)

### Step 7. tert-butyl N-(7-hydroxybenzofuran-5-yl)carbamate

To a solution of tert-butyl N-[7-(4,4,5,5-tetramethyl-1,3,2-dioxaborolan-2-yl)benzofuran-5-yl]carbamate (6.26 g, 17.43 mmol) in EtOH (100 mL) was added H₂O₂ (4.94 g, 43.57 mmol, 30% purity) at 0° C., then the mixture was stirred at 20° C. for 2 h. TLC (petroleum ether: ethyl acetate=3:1, Rf=0.5) indicated starting material was consumed completely, and one major new spot was detected. LCMS showed starting material was consumed completely and mass of the desired compound. The reaction was added saturated sodium sulfite solution (100 mL) stirred at 20° C. for 10 min, then was added water (50 mL), then extracted with ethyl acetate (3×100 mL) and dried over anhydrous sodium sulfate, filtered and concentrated under reduced pressure to give a residue. The residue was purified by flash chromatography (silica gel, petroleum ether/ethyl acetate=1/0 to 3/1) to give tert-butyl N-(7-hydroxybenzofuran-5-yl)carbamate (4.5 g, crude) as a brown oil.

¹H NMR (400 MHz, METHANOL-d₄) δ ppm 1.52 (s, 9H) 6.70 (d, J=2.13 Hz, 1H) 6.84 (d, J=1.25 Hz, 1H) 7.11 (s, 1H) 7.65 (d, J=2.00 Hz, 1H)

### Step 8. tert-butyl N-[7-(3-pyrrolidin-1-yl]propoxy)benzofuran-5-yl]carbamate

To a solution of tert-butyl N-(7-hydroxybenzofuran-5-yl)carbamate (4.5 g, 18.05 mmol), 1-(3-chloropropyl)pyrrolidine (2.40 g, 16.25 mmol) and Cs₂CO₃ (11.76 g, 36.11 mmol) in MeCN (50 mL) was added NaI (2.71 g, 18.05 mmol, 737.95 μL), then the mixture was stirred at 70° C. for 2 h. LCMS showed starting material was consumed completely and mass of the desired compound. The reaction was filtered and concentrated under reduced pressure to give tert-butyl N-[7-(3-pyrrolidin-1-ylpropoxy)benzofuran-5-yl]carbamate (6.5 g, crude) as a brown oil.

### Step 9. 7-(3-pyrrolidin-1-ylpropoxy)benzofuran-5-amine

To a solution of tert-butyl N-[7-(3-pyrrolidin-1-ylpropoxy)benzofuran-5-yl]carbamate (6.5 g, 18.03 mmol) in DCM (60 mL) was added TFA (30 mL), then the mixture was stirred at 20° C. for 12 h. TLC (ethyl acetate:methanol=5:1, Rf=0.3) indicated starting material was consumed completely, and one major new spot was detected. LCMS showed starting material was consumed completely and mass of the desired compound. The reaction was filtered and concentrated under reduced pressure to give a residue. The crude product was purified by reversed-phase HPLC (0.1% TFA condition) to give 7-(3-pyrrolidin-1-ylpropoxy)benzofuran-5-amine (4.7 g, crude) as a brown gum.

### Step 10. 7-(3-pyrrolidin-1-ylpropoxy)-2,3-dihydrobenzofuran-5-amine

To a mixture 7-(3-pyrrolidin-1-ylpropoxy)benzofuran-5-amine (1.3 g, 4.99 mmol) in MeOH (250 mL) was added Pd/C (2 g, purity: 10%) at 20° C., then the reaction mixture was stirred at 30° C. for 12 h under H2 (50 Psi). LCMS showed starting material was consumed completely and mass of the desired compound. Two reactions were combined. The reaction was filtered and concentrated under reduced pressure to give a residue. The residue was purified by reversed-phase HPLC (0.1% TFA condition) to give 7-(3-pyrrolidin-1-ylpropoxy)-2,3-dihydrobenzofuran-5-amine (2.99 g, 7.97 mmol, 79.76% yield, TFA) as a brown gum.

¹H NMR (400 MHz, METHANOL-d₄) δ ppm 2.05 (m, 2H) 2.18 (br s, 2H) 2.24 (m, 2H) 3.12 (m, 2H) 3.27 (m, 2H) 3.43 (m, 2H) 3.75 (br s, 2H) 4.20 (t, J=5.63 Hz, 2H) 4.66 (t, J=8.82 Hz, 2H) 6.84 (d, J=1.88 Hz, 1H) 6.90 (d, J=1.00 Hz, 1H)

### Step 11. N⁴,6-dimethyl-N²-[7-(3-pyrrolidin-1-ylpropoxy)-2,3-dihydrobenzofuran-5-yl]pyrimidine-2,4-diamine

To a solution of 7-(3-pyrrolidin-1-ylpropoxy)-2,3-dihydrobenzofuran-5-amine (100 mg, 266.41 μmol, TFA) and 2-chloro-N,6-dimethyl-pyrimidin-4-amine (41.99 mg, 266.41 μmol) in i-PrOH (2 mL) was added TFA (3.04 mg, 26.64 μmol, 2.05 μL), then the mixture was stirred at 140° C. for 1 h. LCMS showed starting material was consumed completely and mass of the desired compound. Eight reactions were combined. The reaction was added ethyl acetate (30 mL), the mixture was stirred at 20° C. for 10 min. The mixture was filtered and the filter cake was concentrated under reduced pressure to give N⁴,6-dimethyl-N²-[7-(3-pyrrolidin-1-ylpropoxy)-2,3-dihydrobenzofuran-5-yl]pyrimidine-2,4-diamine (742.6 mg, 1.50 mmol, 70.18% yield, TFA) (purity: 97.547%) as an off-white solid. It was rechecked by LCMS and HNMR.

¹H NMR (400 MHz, METHANOL-d₄) δ ppm 2.06 (br d, J=1.00 Hz, 2H) 2.19 (br s, 2H) 2.22 (s, 2H) 2.28 (s, 3H) 2.97 (s, 3H) 3.13 (m, 2H) 3.26 (brt, J=8.69 Hz, 2H) 3.44 (m, 2H) 3.75 (br s, 2H) 4.19 (t, J=5.57 Hz, 2H) 4.63 (t, J=8.76 Hz, 2H) 5.96 (d, J=0.63 Hz, 1H) 7.06 (br d, J=6.00 Hz, 2H). MS (ESI): m/z=384.2 [M+H]⁺

### EXAMPLE 3. Synthesis of Compound 104

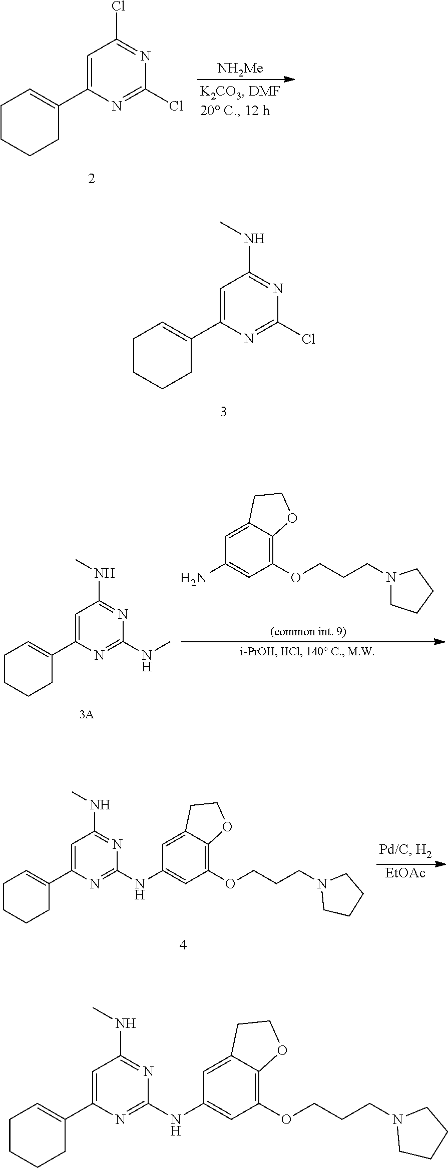

### Step 1. 2-chloro-6-(cyclohexen-1-yl)-N-methyl-pyrimidin-4-amine

To a mixture of 2,4-dichloro-6-(cyclohexen-1-yl)pyrimidine (700 mg, 3.06 mmol) in DMF (10.05 mL) was added tripotassium carbonate (1.69 g, 12.22 mmol, 737.59 μL) and methanamine hydrochloride (247.55 mg, 3.67 mmol) at 25° C., then the mixture was stirred at 25° C. for 12 hrs. The reaction mixture was added to water (10 mL), extracted with EtOAc (10 mL*3). The organic layer was dried over Na₂SO₄, concentrated to give the crude product. The crude product was purified by column chromatography (SiO₂, Petroleum ether/Ethyl acetate=1/0 to 20/1) to give 2-chloro-6-(cyclohexen-1-yl)-N-methyl-pyrimidin-4-amine (371.6 mg, crude) as a white solid and 4-chloro-6-(cyclohexen-1-yl)-N-methyl-pyrimidin-2-amine (100 mg, crude) as a white solid.

### Step 2. 6-(cyclohexen-1-yl)-N4-methyl-N2-[7-(3-pyrrolidin-1-ylpropoxy)-2,3-dihydrobenzofuran-5-yl]pyrimidine-2,4-diamin

To a mixture of 2-chloro-6-(cyclohexen-1-yl)-N-methyl-pyrimidin-4-amine (50 mg, 223.51 μmol) and 7-(3-pyrrolidin-1-ylpropoxy)-2,3-dihydrobenzofuran-5-amine (58.64 mg, 223.51 μmol) in i-PrOH (2 mL) was added HCl (814.93 μg, 22.35 μmol, 1.02e-3 μL) at 25° C., then the reaction vessel was sealed and heated in microwave at 140° C. for 3 h. LCMS showed the desired mass was detected. The crude product was purified by prep-HPLC(TFA condition:Phenomenex Luna 80*30 mm*3 um; mobile phase:water(0.1% TFA)-ACN; B %: 15%-40%, 8 min) to give 6-(cyclohexen-1-yl)-N4-methyl-N2-[7-(3-pyrrolidin-1-ylpropoxy)-2,3-dihydrobenzofuran-5-yl]pyrimidine-2,4-diamine (15.61 mg, crude) as a yellow solid.

### Step 3. 6-cyclohexyl-N4-methyl-N2-[7-(3-pyrrolidin-1-ylpropoxy)-2,3-dihydrobenzofuran-5-yl]pyrimidine-2,4-diamine

To a mixture of 6-(cyclohexen-1-yl)-N4-methyl-N2-[7-(3-pyrrolidin-1-ylpropoxy)-2,3-dihydrobenzofuran-5-yl]pyrimidine-2,4-diamine (100.49 mg, 223.52 μmol) in MeOH (3 mL) was added Pd/C (30 mg, 10% purity) at 25° C., then the mixture was stirred at 25° C. for 3 hrs under H₂. LCMS showed the desired mass was detected. The reaction was filtered and concentrated under reduced pressure to give a crude product. The crude product was purified by prep-HPLC(TFA condition:column:Phenomenex Luna 80*30 mm*3 um; mobile phase:water(0.1% TFA)-ACN]; B %: 10%-40%, 8 min) to give 6-cyclohexyl-N4-methyl-N2-[7-(3-pyrrolidin-1-ylpropoxy)-2,3-dihydrobenzofuran-5-yl]pyrimidine-2,4-diamine (17.9 mg, 39.64 μmol, 17.73% yield) as a white solid.

¹H NMR (400 MHz, METHANOL-d₄) δ ppm 1.25-1.38 (m, 2H) 1.40-1.49 (m, 4H) 1.75-1.81 (m, 1H) 1.87-1.97 (m, 4H) 2.01-2.09 (m, 2H) 2.09-2.29 (m, 5H) 2.45-2.57 (m, 1H) 2.96-2.99 (m, 3H) 3.07-3.18 (m, 2H) 3.22-3.29 (m, 2H) 3.44 (br t, J=7.32 Hz, 2 H)3.69-3.80 (m, 2H)4.19 (t, J=5.44 Hz, 2H)4.63 (t, J=8.69 Hz, 2H)5.94-5.97 (m, 1H) 7.07-7.12 (m, 2H).

### EXAMPLE 4. Synthesis of Compound 105

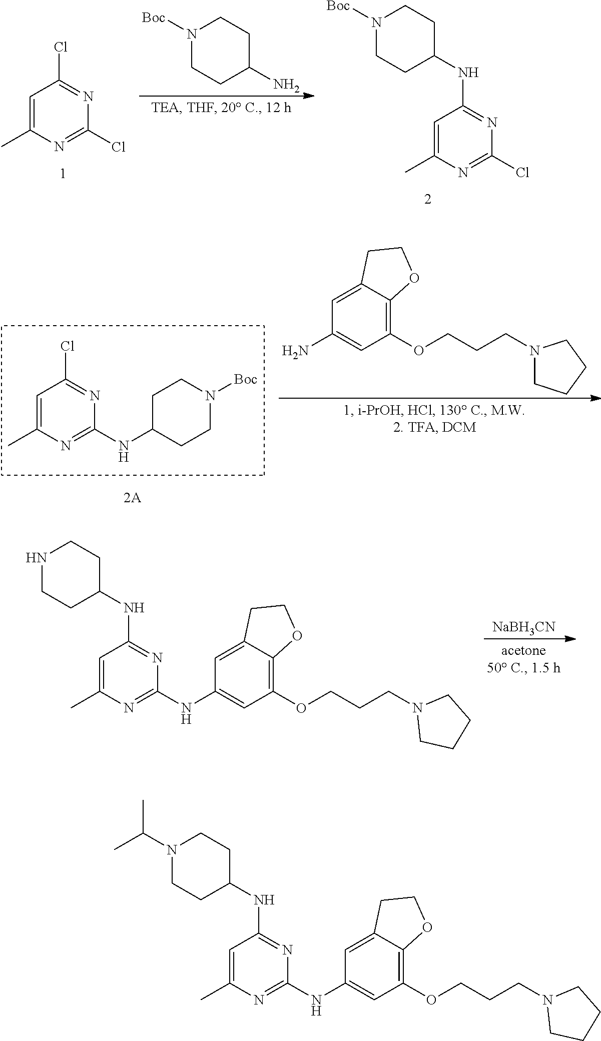

### Step 1. tert-butyl 4-[(2-chloro-6-methyl-pyrimidin-4-yl)amino]piperidine-1-carboxylate

To a solution of 2,4-dichloro-6-methyl-pyrimidine (1 g, 6.13 mmol), tert-butyl 4-aminopiperidine-1-carboxylate (1.72 g, 8.59 mmol) and TEA (931.17 mg, 9.20 mmol, 1.28 mL) in THF (19.99 mL). It was stirred at 25° C. for 12 h. LCMS showed starting material was consumed completely and mass of the desired compound. The residue was purified by flash chromatography (silica gel, petroleum ether/ethyl acetate=1/0 to 0/1) to give tert-butyl 4-[(2-chloro-6-methyl-pyrimidin-4-yl)amino]piperidine-1-carboxylate (1.36 g, crude) and tert-butyl-4-[(6-chloro-2-methyl-pyrimidin-4-yl)amino]piperidine-1-carboxylate (510 mg, crude).

### Step 2. tert-butyl 4-[(2-chloro-6-methyl-pyrimidin-4-yl)amino]piperidine-1-carboxylate

To the mixture of tert-butyl 4-[(2-chloro-6-methyl-pyrimidin-4-yl)amino]piperidine-1-carboxylate (50 mg, 152.99 mol) and 7-(3-pyrrolidin-1-ylpropoxy)-2,3-dihydrobenzofuran-5-amine (40.14 mg, 152.99 μmol) in i-PrOH (1 mL) was added HCl (557.80 μg, 15.30 μmol) (12 M), then the mixture was stirred at 130° C. in the microwave for 1 hrs. LCMS showed starting material was consumed completely and mass of the desired compound. The reaction mixture was filtered and the filter cake was concentrated to get 6-methyl-N4-(1-methyl-4-piperidyl)-N2-[7-(3-pyrrolidin-1-ylpropoxy)-2,3-dihydrobenzofuran-5-yl]pyrimidine-2,4-diamine (60 mg, 128.59 μmol, 84.05% yield) as a brown solid. The solution of tert-butyl 4-[[6-methyl-2-[[7-(3-pyrrolidin-1-ylpropoxy)-2,3-dihydrobenzofuran-5-yl]amino]pyrimidin-4-yl]amino]piperidine-1-carboxylate (0.05 g, 90.46 mol) in DCM (1 mL) and TFA (10.31 mg, 90.46 μmol, 6.97 μL). It was stirred at 20° C. for 1 h. showed starting material was consumed completely and mass of the desired compound. The reaction was concentrated under reduced pressure. It was purified by prep-HPLC (TFA condition, column: Phenomenex luna C18 100*40 mm*5 um; mobile phase: [water(0.1% TFA)-ACN]; B %: 1%-25%,8 min) to give 6-methyl-N4-(piperidin-4-yl)-N2-(7-(3-(pyrrolidin-1-yl)propoxy)-2,3-dihydrobenzofuran-5-yl)pyrimidine-2,4-diamine (50 mg, 110.48 mol) as a white solid.

¹H NMR (400 MHz, METHANOL-d₄) δ ppm 1.75-1.87 (m, 2H) 2.02-2.12 (m, 2H) 2.13-2.28 (m, 7H) 2.33 (br s, 4H) 3.05-3.16 (m, 3H) 3.28 (br t, J=8.72 Hz, 2H) 3.43-3.52 (m, 4H) 3.69-3.83 (m, 2H) 4.21 (br t, J=5.27 Hz, 2H) 4.66 (t, J=8.66 Hz, 2H) 6.01 (s, 1H) 6.89 (s, 1H) 7.14 (br s, 1H).

### Step 3. N2-(1-isopropyl-4-piperidyl)-6-methyl-N4-[7-(3-pyrrolidin-1-ylpropoxy)-2,3-dihydrobenzofuran-5-yl]pyrimidine-2,4-diamine

To a solution of 6-methyl-N4-(4-piperidyl)-N2-[7-(3-pyrrolidin-1-ylpropoxy)-2,3-dihydrobenzofuran-5-yl]pyrimidine-2,4-diamine (50 mg, 110.48 μmol) in Acetone (1 mL) was added AcOH (663.42 μg, 11.05 μmol) to pH=5, then it was added NaBH₃CN (13.88 mg, 220.95 mol). It was stirred at 50° C. for 1 h. LCMS showed starting material was consumed complete and mass of the desired compound. It was concentrated under reduced pressure to give a residue. It was purified by prep-HPLC (TFA condition, Phenomenex luna C18 100*40 mm*5 um; mobile phase: [water(0.1% TFA)-ACN]; B %: 1%-23%, 8 min,) to give N2-(1-isopropyl-4-piperidyl)-6-methyl-N4-[7-(3-pyrrolidin-1-ylpropoxy)-2,3-dihydrobenzofuran-5-yl]pyrimidine-2,4-diamine (10.7 mg, 21.63 μmol, 9.79% yield) as a brown solid.

¹H NMR (400 MHz, METHANOL-d₄) δ ppm 1.37 (br d, J=6.25 Hz, 6H) 1.84 (br d, J=13.01 Hz, 2H) 2.05 (br d, J=1.25 Hz, 2H) 2.12-2.25 (m, 6H) 2.27-2.32 (m, 3H) 2.37 (br s, 2H) 3.02-3.18 (m, 4H) 3.43 (br t, J=7.25 Hz, 3H) 3.50-3.62 (m, 3H) 3.69-3.78 (m, 2H) 4.16-4.22 (m, 2H)4.61-4.68 (m, 2H)5.92-6.00 (m, 1H) 6.88 (br d, J=16.51 Hz, 1H)7.08 (br s, 1H).

### EXAMPLE 5. Synthesis of Compounds 106 and 107

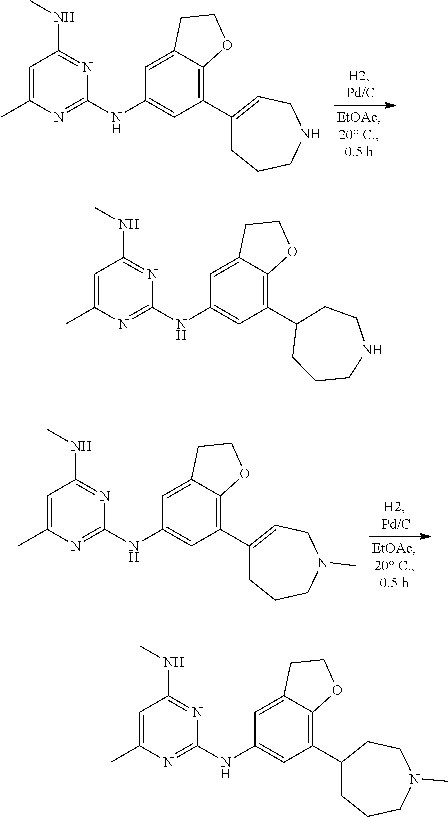

### Step 1. N2-[7-(azepan-4-yl]-2,3-dihydrobenzofuran-5-yl]-N4,6-dimethyl-pyrimidine-2,4-diamine

To a solution of N4,6-dimethyl-N2-[7-(2,3,4,7-tetrahydro-1H-azepin-5-yl)-2,3-dihydrobenzofuran-5-yl]pyrimidine-2,4-diamine (6 mg, 17.07 μmol) in EtOAc (5 mL) was added Pd/C (6 mg, 10% purity). The reaction mixture was stirred at 20° C. under H₂ (15 Psi) for 0.5 h. LCMS showed starting material was consumed complete and mass of the desired compound. It was concentrated under reduced pressure to give a residue. It was purified by prep-HPLC (TFA condition, column: Phenomenex luna C18 100*40 mm*5 um; mobile phase: [water(0.1% TFA)-ACN]; B %: 1%-35%, 8 min) to give N2-[7-(azepan-4-yl)-2,3-dihydrobenzofuran-5-yl]-N4,6-dimethyl-pyrimidine-2,4-diamine (0.2 mg, 5.66e-1 μmol, 3.31% yield)(TFA salt, 100.0% purity) as a pale yellow solid.

¹H NMR (400 MHz, METHANOL-d₄) δ ppm 7.26 (s, 1H) 7.00-7.12 (m, 1H) 5.95 (s, 1H) 4.59-4.63 (m, 2H) 3.72-3.74 (m, 1H) 3.47-3.50 (m, 1H) 3.21-3.26 (m, 4H) 3.13 (t, J=1.65 Hz, 1H) 2.94-2.96 (m, 3H) 2.27 (br s,3 H) 2.06-2.14 (m, 3H) 1.85-1.90 (m, 3H)

### Step 2. N4,6-dimethyl-N2-[7-(1-methylazepan-4-yl)-2,3-dihydrobenzofuran-5-yl]-pyrimidine-2,4-diamine

To a solution of N4,6-dimethyl-N2-[7-(1-methyl-2,3,4,7-tetrahydroazepin-5-yl)-2,3-dihydrobenzofuran-5-yl]pyrimidine-2,4-diamine (9 mg, 24.63 μmol) in EtOAc (5 mL) was added Pd/C (9 mg, 10% purity). The reaction mixture was stirred at 20° C. under H₂ (15 Psi) for 0.5 h. LCMS showed starting material was consumed completely and mass of the desired compound. It was purified by prep-HPLC (TFA condition, column: Phenomenex luna C18 100*40 mm*5 m; mobile phase: [water(0.1% TFA)-ACN]; B %: 1%-15%, 8 min) to give N4,6-dimethyl-N2-[7-(1-methylazepan-4-yl)-2,3-dihydrobenzofuran-5-yl]pyrimidine-2,4-diamine (3.3 mg, 8.98 μmol, 36.47% yield)(TFA salt, 93.178% purity) as a white solid.

¹H NMR (400 MHz, METHANOL-d₄) δ ppm 7.34 (br s, 1H) 7.11 (br s, 1H) 5.96 (s, 1H) 4.56-4.66 (m, 2H) 3.57 (br dd, J=13.01, 7.75 Hz, 2H) 3.20-3.30 (m, 3H) 3.09 (br dd, J=6.63, 3.50 Hz, 1H) 2.97 (d, J=8.88 Hz, 6H) 2.29 (s, 3H) 2.23 (br s, 1H) 2.10 (br s, 3H) 1.84-2.05 (m, 2H).

### EXAMPLE 6. Synthesis of Compound 108

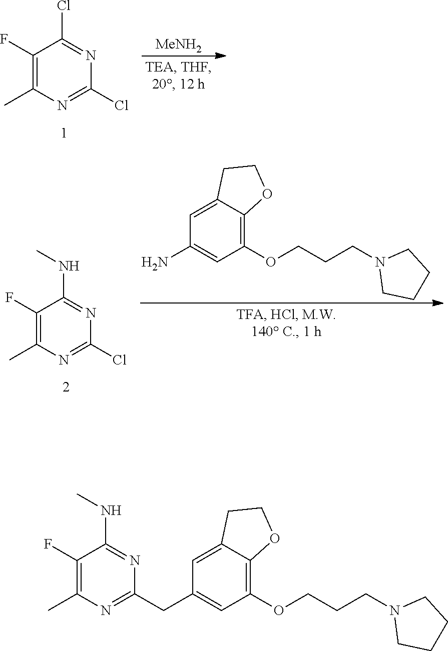

### Step 1. 2-chloro-5-fluoro-N,6-dimethyl]pyrimidin-4-amine

To a solution of 2,4-dichloro-5-fluoro-6-methyl-pyrimidine (900 mg, 4.97 mmol) and methanamine (402.88 mg, 5.97 mmol, 448.14 μL, HCl) in THF (18 mL) was added N,N-diethylethanamine (1.51 g, 14.92 mmol, 2.08 mL), then the mixture was stirred 20° C. for 12 hrs. LCMS showed the reaction was complete and the desired ms was detected. The reaction was concentrated under reduced pressure to give a residue. The residue was purified by column chromatography (SiO₂, Petroleum ether/Ethyl acetate=1/0 to 10/1) to give 2-chloro-5-fluoro-N,6-dimethyl-pyrimidin-4-amine (560 mg, crude) as white solid.

### Step 2. 5-fluoro-N4,6-dimethyl-N2-(7-(3-(pyrrolidin-1-yl)propoxy)-2,3-dihydrobenzofuran-5-yl) pyrimidine-2,4-diamine

To the mixture of 2-chloro-5-fluoro-N,6-dimethyl-pyrimidin-4-amine (20 mg, 113.90 μmol) and 7-(3-pyrrolidin-1-ylpropoxy)-2,3-dihydrobenzofuran-5-amine (29.88 mg, 113.90 μmol) in i-PrOH (2 mL) was added HCl (12 M, 9.49 e-1 μL), then the mixture was stirred at 140° C. in the microwave for 1 hr. LCMS showed the reaction was complete and the desired ms was detected. The reaction was filtered and concentrated under reduced pressure to give a residue. The crude product was purified by prep-HPLC (TFA condition:column:Phenomenex luna C18 100*40 mm*5 um; mobile phase:water(0.1% TFA)-ACN; B %: 1%-25%, 8 min) to give 5-fluoro-N4,6-dimethyl-N2-[7-(3-pyrrolidin-1-ylpropoxy)-2,3-dihydrobenzofuran-5-yl]pyrimidine-2,4-diamine (17 mg, 33.04 μmol, 29.01% yield, TFA) as yellow solid.

¹H NMR (400 MHz, METHANOL-d₄) δ ppm 7.07 (s, 1H), 7.01 (s, 1H), 4.69-4.56 (m, 2H), 4.19 (t, J=5.6 Hz, 2H), 3.87-3.67 (m, 2H), 3.47-3.41 (m, 2H), 3.28-3.23 (m, 2H), 3.20-3.08 (m, 2H), 3.03 (s, 3H), 2.32 (d, J=2.9 Hz, 3H), 2.27-2.14 (m, 4H), 2.12-1.99 (m, 2H)

MS (ESI): m/z=402.1 [M+H].

### EXAMPLE 7. Synthesis of Compound 109

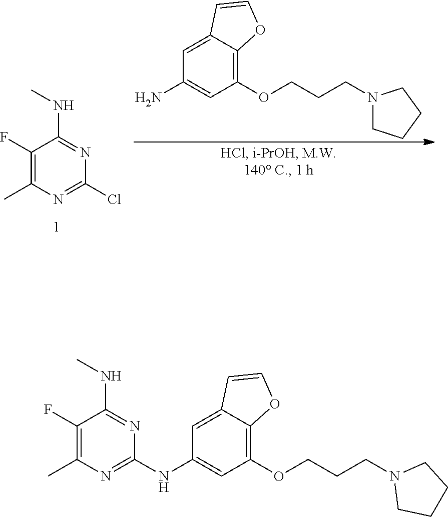 [SMILES: CNc1nc(Cl)nc(C)c1F ; Nc1cc(OCCCN2CCCC2)c2occc2c1 ; CNc1nc(Nc2cc(OCCCN3CCCC3)c3occc3c2)nc(C)c1F]

### 5-fluoro-N4,6-dimethyl-N2-[7-(3-pyrrolidin-1-ylpropoxy)benzofuran-5-yl]pyrimidine-2,4-diamine-2,4-diamine

To the mixture of 2-chloro-5-fluoro-N,6-dimethyl-pyrimidin-4-amine (30 mg, 170.85 μmol) and 7-(3-pyrrolidin-1-ylpropoxy)benzofuran-5-amine (44.48 mg, 170.85 μmol) in i-PrOH (3 mL) was added HCl (12 M, 1.42 μL), then the mixture was stirred at 140° C. in the microwave for 1 hrs. LCMS showed the reaction was complete and the desired ms was detected. The reaction was filtered and concentrated under reduced pressure to give a residue. The crude product was purified by prep-HPLC(TFA condition:Phenomenex luna C18 100*40 mm*5 um; mobile phase:water(0.1% TFA)-ACN; B %: 1%-32%, 8 min) to give 5-fluoro-N4,6-dimethyl-N2-[7-(3-pyrrolidin-1-ylpropoxy)benzofuran-5-yl]pyrimidine-2,4-diamine (64.9 mg, 126.64 μmol, 74.12% yield, TFA) as pale yellow solid.

¹H NMR (400 MHz, METHANOL-d₄) 6=7.80 (d, J=2.0 Hz, 1H), 7.49 (s, 1H), 7.08 (s, 1H), 6.87 (d, J=2.1 Hz, 1H), 4.35 (t, J=5.5 Hz, 2H), 3.77 (br s, 2H), 3.53-3.46 (m, 2H), 3.24-3.09 (m, 2H), 3.05 (s, 3H), 2.33 (br d, J=2.8 Hz, 5H), 2.19 (br s, 2H), 2.07 (br s, 2H). MS (ESI): m/z=400.1 [M+H]

### EXAMPLE 8. Synthesis of Compounds 110 and 111

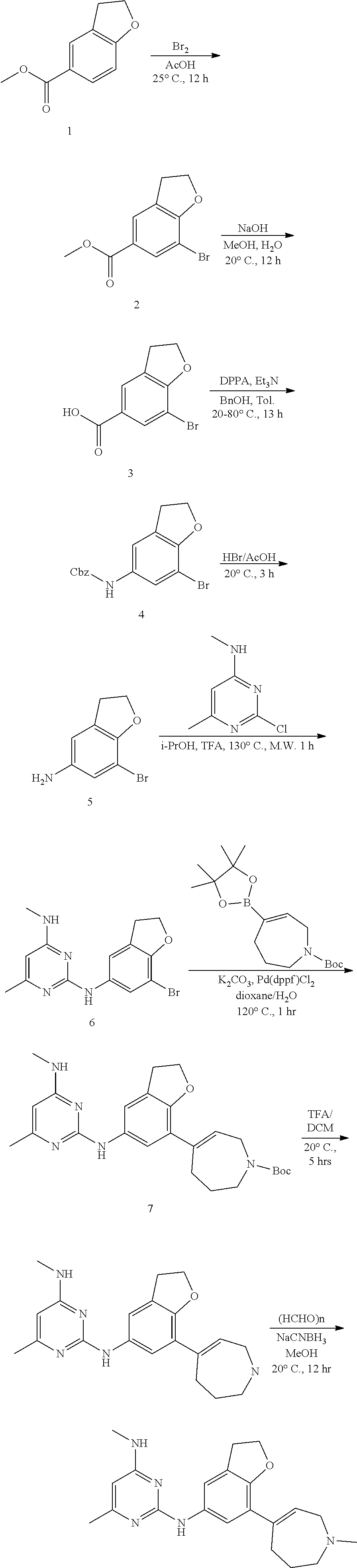

### Step 1. methyl 7-bromo-2,3-dihydrobenzofuran-5-carboxylate

To a mixture of methyl 2,3-dihydrobenzofuran-5-carboxylate (10 g, 56.12 mmol) in AcOH (200 mL) was added drop wise Br₂ (22.42 g, 140.30 mmol) at 25° C., then the mixture was stirred at 25° C. for 12 hrs. LCMS showed the reaction was complete mostly and the desired ms was detected. The reaction mixture was added to sat. Na₂S₂O₃ aq (100 mL), extracted with EtOAc (300 mL*3). The combined organic layers were washed with 600 mL of brine, dried over Na₂SO₄, filtered and concentrated under reduced pressure to give methyl 7-bromo-2,3-dihydrobenzofuran-5-carboxylate (16.5 g, crude) as a yellow solid.

### Step 2. 7-bromo-2,3-dihydrobenzofuran-5-carboxylic acid

To a solution of methyl 7-bromo-2,3-dihydrobenzofuran-5-carboxylate (16.5 g, 64.18 mmol) in H₂O (40 mL), MeOH (165 mL) was added NaOH (5.13 g, 128.36 mmol, 2.41 mL). It was stirred at 20° C. for 12 h. LCMS showed the reaction was complete mostly and the desired ms was detected. It was concentrated under reduced pressure, then added dropwise 1N HCl to pH=1. It was filtered to get a filter cake. Compound 7-bromo-2,3-dihydrobenzofuran-5-carboxylic acid (10.54 g, crude) was obtained as a yellow solid.

### Step 3. Benzyl N-(7-bromo-2,3-dihydrobenzofuran-5-yl)carbamate

To a solution of 7-bromo-2,3-dihydrobenzofuran-5-carboxylic acid (3.34 g, 13.74 mmol) in Tol. (80 mL) was added DPPA (4.01 g, 16.49 mmol), TEA (4.17 g, 41.23 mmol, 5.75 mL). It was stirred at 20° C. for 1 h. Then it was added BnOH (1.85 g, 41.23 mmol). It was stirred at 80° C. for 12 h. TLC (petroleum ether:ethyl acetate=3:1, Rf=0.6) indicated starting material was consumed completely, and one major new spot was detected. The three batches of the reaction mixture were mixed together for work up. It was concentrated under reduced pressure to give a residue. The residue was purified by flash chromatography (silica gel, petroleum ether/ethyl acetate=1/0 to 1/1) to give benzyl N-(7-bromo-2,3-dihydrobenzofuran-5-yl)carbamate (5.79 g, crude) as a yellow oil.

### Step 4. 7-bromo-2,3-dihydrobenzofuran-5-amine

To a solution of benzyl N-(7-bromo-2,3-dihydrobenzofuran-5-yl)carbamate (2 g, 5.74 mmol) in AcOH (15 mL) was added HBr (30 mL). The reaction mixture was stirred at 20° C. for 3 h. LCMS showed starting material was consumed completely and mass of the desired compound. The reaction was extracted with ethyl acetate (50 mL*3). The aqueous phase were added NaOH to pH=8 at 0° C., then extracted with ethyl acetate (50 mL*3). The combined organic layers were washed with brine (3×50 mL) and dried over anhydrous sodium sulfate, filtered and concentrated under reduced pressure to give 7-bromo-2,3-dihydrobenzofuran-5-amine (540 mg, crude) as a yellow gum.

### Step 5. N2-(7-bromo-2,3-dihydrobenzofuran-5-yl)-N4,6-dimethyl-pyrimidine-2,4-diamine

To a solution of 2-chloro-N,6-dimethyl-pyrimidin-4-amine (294.50 mg, 1.87 mmol) and 7-bromo-2,3-dihydrobenzofuran-5-amine (400 mg, 1.87 mmol) in i-PrOH (5 mL) was added TFA (21.31 mg, 186.86 μmol, 14.40 μL). The reaction mixture was stirred at 130° C. for 1 h. LCMS showed starting material was consumed completely and mass of the desired compound. The four batches of the reaction mixture were mixed together for work up. The reaction mixture was filtered to give N2-(7-bromo-2,3-dihydrobenzofuran-5-yl)-N4,6-dimethyl-pyrimidine-2,4-diamine (1.9 g, crude) (TFA salt) as a brown solid.

### Step 6. tert-butyl 5-[5-[[4-methyl-6-(methylamino)pyrimidin-2-yl]amino]-2,3-dihydrobenzofuran-7-yl]-2,3,4,7-tetrahydroazepine-1-carboxylate

A mixture of N2-(7-bromo-2,3-dihydrobenzofuran-5-yl)-N4,6-dimethyl-pyrimidine-2,4-diamine (50 mg, 149.17 μmol), tert-butyl 5-(4,4,5,5-tetramethyl-1,3,2-dioxaborolan-2-yl)-2,3,4,7-tetrahydroazepine-1-carboxylate (62.68 mg, 193.91 mol), K2CO₃ (41.23 mg, 298.33 μmol) and Pd(dppf)Cl₂ (21.83 mg, 29.83 mol) in H₂O (0.5 mL) and dioxane (5 mL) was degassed and purged with N2 for 3 times, and then the mixture was stirred at 120° C. for 1 hr under microwave heating under N₂ atmosphere. LCMS showed the reaction was complete. The eleven batches of the reaction mixture were mixed together for work up. The reaction mixture was filtered and concentrated under reduced pressure to give tert-butyl5-[5-[[4-methyl-6-(methylamino)pyrimidin-2-yl]amino]-2,3-dihydrobenzofuran-7-yl]-2,3,4,7-tetrahydroazepine-1-carboxylate (1.1 g, crude) as a brown solid.

### Step 7. N4,6-dimethyl-N2-[7-(2,3,4,7-tetrahydro-1H-azepin-5-yl)-2,3-dihydrobenzofuran-5-yl]pyrimidine-2,4-diamine

To a solution of tert-butyl 5-[5-[[4-methyl-6-(methylamino)pyrimidin-2-yl]amino]-2,3-dihydrobenzofuran-7-yl]-2,3,4,7-tetrahydroazepine-1-carboxylate (850 mg, 1.88 mmol) in DCM (10 mL) was added TFA (10 mL). The mixture was stirred at 20° C. for 5 h. LCMS showed the reaction was complete. The reaction mixture was filtered and concentrated under reduced pressure to give a crude product. The residue was purified by prep-HPLC (TFA condition; column: Phenomenex luna C18 250*50 mm*10 um; mobile phase: [water(0.1% TFA)-ACN]; B %: 5%-35%,10 min). Compound N4,6-dimethyl-N2-[7-(2,3,4,7-tetrahydro-1H-azepin-5-yl)-2,3-dihydrobenzofuran-5-yl]pyrimidine-2,4-diamine (50.5 mg, 108.73 μmol, 5.78% yield, TFA, purity: 100%) was obtained as a pale yellow solid. N4,6-dimethyl-N2-[7-(2,3,4,7-tetrahydro-1H-azepin-5-yl)-2,3-dihydrobenzofuran-5-yl]pyrimidine-2,4-diamine (132 mg, crude, TFA) was obtained as a pale yellow solid.

¹H NMR (400 MHz, METHANOL-d₄) δ ppm 7.44-7.38 (m, 1H), 7.24-7.17 (m, 1H), 6.13-6.07 (m, 1H), 5.95 (s, 1H), 4.63-4.59 (m, 2H), 3.92-3.86 (m, 2H), 3.49-3.44 (m, 2H), 3.27-3.23 (m, 2H), 2.96 (s, 3H), 2.88-2.80 (m, 2H), 2.29-2.27 (m, 3H), 2.09-2.01 (m, 2H)

### Step 8. N4,6-dimethyl-N2-[7-(1-methyl-2,3,4,7-tetrahydroazepin-5-yl]-2,3-dihydrobenzofuran-5-yl]pyrimidine-2,4-diamine

To a solution of N4,6-dimethyl-N2-[7-(2,3,4,7-tetrahydro-1H-azepin-5-yl)-2,3-dihydrobenzofuran-5-yl]pyrimidine-2,4-diamine (167 mg, 475.18 mol) in MeOH (5 mL) was added DIEA adjust to pH=7-8, AcOH was added to above solution to adjust pH=5-6. Sodium cyanoboranuide (59.72 mg, 950.36 mol) and (HCHO)ₙ(2.85 g, 2.38 mmol) was added to above solution. Then the mixture was stirred at 20° C. for 12 hr. LCMS showed the reaction was complete. The reaction mixture was filtered and concentrated under reduced pressure to give a crude product. The residue was purified by prep-HPLC (TFA condition; column: Phenomenex luna C18 250*50 mm*10 um; mobile phase: [water(0.1% TFA)-ACN]; B %: 1%-30%, 10 min) to give N4,6-dimethyl-N2-[7-(1-methyl-2,3,4,7-tetrahydroazepin-5-yl)-2,3-dihydrobenzofuran-5-yl]pyrimidine-2,4-diamine (124.3 mg, 259.78 μmol, 54.67% yield, TFA) as a white solid.

¹H NMR (400 MHz, METHANOL-d₄) δ ppm 7.46-7.40 (m, 1H), 7.27-7.21 (m, 1H), 6.09-6.01 (m, 1H), 5.95 (s, 1H), 4.64-4.58 (m, 2H), 4.14-3.89 (m, 2H), 3.70-3.44 (m, 2H), 3.25 (br t, J=8.6 Hz, 2H), 2.96 (s, 3H), 2.92 (s, 3H), 2.88-2.82 (m, 2H), 2.30-2.25 (m, 3H), 2.08 (br s, 2H).

### EXAMPLE 9. Synthesis of Compound 112

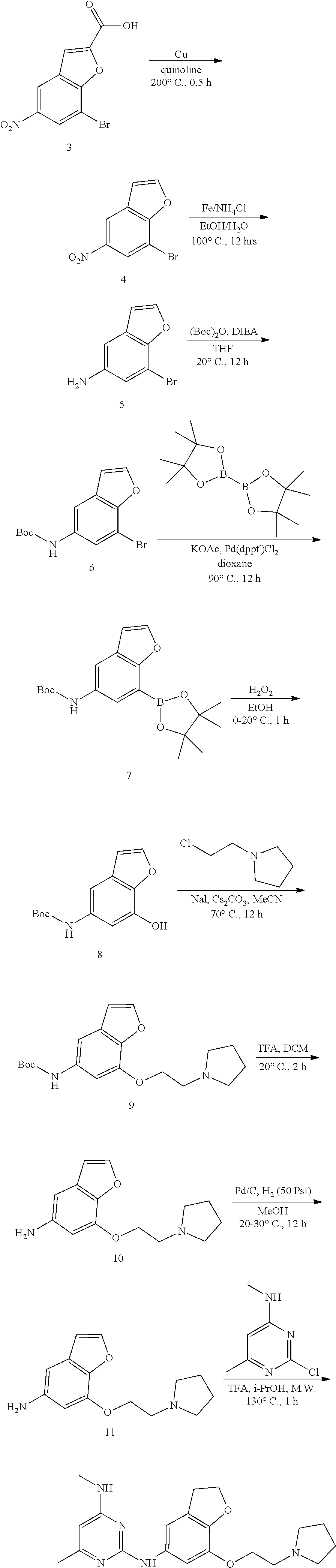

### Step 1. 7-bromo-5-nitro-benzofuran

To a mixture of 7-bromo-5-nitro-benzofuran-2-carboxylic acid (550 mg, 1.92 mmol) in quinoline (5 mL) was added Cu (244.39 mg, 3.85 mmol) at 20° C., then the mixture was stirred at 200° C. for 0.5 h. TLC(petroleum ether:ethyl acetate=3:1, Rf=0.7) indicated starting material was consumed completely, and three major new spots were detected. Eight reactions were combined. To the combined mixture was added 12 M HCl (150 mL) stirred at 20° C. for 15 min. Then extracted with ethyl acetate (200*3 mL). The combined organic layers were washed with brine (200*3 mL) and dried over anhydrous sodium sulfate, filtered and concentrated under reduced pressure to give 7-bromo-5-nitro-benzofuran (6.4 g, crude) as a brown oil.

### Step 2. 7-bromobenzofuran-5-amine

To a solution of 7-bromo-5-nitro-benzofuran (2.9 g, 11.98 mmol), NH₄Cl (6.41 g, 119.82 mmol) and H₂O (6 mL) in EtOH (60 mL) was added Fe (3.35 g, 59.91 mmol, 425.66 uL). It was stirred at 100° C. for 12 h. TLC (petroleum ether:ethyl acetate=3:1, Rf=0.5) indicated starting material was consumed completely, and two major new spots were detected. The reaction was filtered and concentrated under reduced pressure, then added water (200 mL), then extracted with ethyl acetate (200*3 mL) and dried over anhydrous sodium sulfate, filtered and concentrated under reduced pressure to give 7-bromobenzofuran-5-amine (4.28 g, crude) as a brown oil.

### Step 3. tert-butyl N-(7-bromobenzofuran-5-yl)carbamate

To a solution of 7-bromobenzofuran-5-amine (2 g, 9.43 mmol) and DIEA (2.44 g, 18.86 mmol, 3.29 mL) in THF (20 mL) was added di-tert-butyl dicarbonate (2.47 g, 11.32 mmol, 2.60 mL). It was stirred at 20° C. for 12 h. TLC(petroleum ether:ethyl acetate=3:1, Rf=0.8) indicated starting material was consumed completely, and one major new spot was detected. LCMS showed starting material was consumed completely and mass of the desired compound. The reaction was concentrated under reduced pressure. The residue was purified by flash chromatography (silica gel, petroleum ether/ethyl acetate=1/0 to 19/1) to give tert-butyl N-(7-bromobenzofuran-5-yl)carbamate (2.03 g, 6.50 mmol, 68.95% yield) as a brown oil.

### Step 4. tert-butyl N-[7-(4,4,5,5-tetramethyl-1,3,2-dioxaborolan-2-yl)benzofuran-5-yl]carbamate

To a solution of tert-butyl N-(7-bromobenzofuran-5-yl)carbamate (2.03 g, 6.50 mmol),4,4,5,5-tetramethyl-2-(4,4,5,5-tetramethyl-1,3,2-dioxaborolan-2-yl)-1,3,2-dioxaborolane (1.98 g, 7.80 mmol) and CH₃COOK (1.28 g, 13.01 mmol) in dioxane (40 mL) was added Pd(dppf)Cl₂ (475.83 mg, 650.31 umol) under an atmosphere of nitrogen. It was stirred at 90° C. for 12 hs under an atmosphere of nitrogen. LCMS showed starting material was consumed completely and mass of the desired compound. The reaction was filtered and concentrated under reduced pressure to give a residue. The residue was purified by flash chromatography (silica gel, petroleum ether/ethyl acetate=1/0 to 3/2) to give tert-butyl N-[7-(4,4,5,5-tetramethyl-1,3,2-dioxaborolan-2-yl)benzofuran-5-yl]carbamate (1.92 g, 5.34 mmol, 82.19% yield) as a light-yellow solid.

### Step 5. tert-butyl N-(7-hydroxybenzofuran-5-yl)carbamate

To a solution of tert-butyl N-[7-(4,4,5,5-tetramethyl-1,3,2-dioxaborolan-2-yl)benzofuran-5-yl]carbamate (3.17 g, 8.82 mmol) in EtOH (30 mL) was added H₂O₂ (2.00 g, 17.65 mmol, 30% purity) at 0° C., then the mixture was stirred at 20° C. for 1 h. TLC (petroleum ether:ethyl acetate=5:1, Rf=0.3) indicated starting material was consumed completely, and two major new spots were detected. LCMS showed starting material was consumed completely and mass of the desired compound. The reaction was added saturated sodium sulfite solution (30 mL) stirred at 20° C. for 10 min, then was added water (50 mL), then extracted with ethyl acetate (50*3 mL) and dried over anhydrous sodium sulfate, filtered and concentrated under reduced pressure to give a residue. The residue was purified by flash chromatography (silica gel, petroleum ether/ethyl acetate=1/0 to 3/1) to give tert-butyl N-(7-hydroxybenzofuran-5-yl)carbamate (2.3 g, crude) as a brown oil.

### Step 6. tert-butyl N-[7-(2-pyrrolidin-1-ylethoxy)benzofuran-5-yl]carbamate

A mixture of tert-butyl N-(7-hydroxybenzofuran-5-yl)carbamate (200 mg, 802.37 μmol), 1-(2-chloroethyl)pyrrolidine (96.49 mg, 722.13 μmol), NaI (108.24 mg, 722.13 μmol, 29.52 μL) and C_{S2}CO₃ (784.28 mg, 2.41 mmol) in MeCN (8 mL) then the mixture was stirred at 70° C. for 12 hr. LCMS showed the reaction was complete. The reaction mixture was filtered and concentrated under reduced pressure to give tert-butyl N-[7-(2-pyrrolidin-1-ylethoxy)benzofuran-5-yl]carbamate (277 mg, crude) as purple oil.

### Step 7. 7-(2-pyrrolidin-1-ylethoxy)benzofuran-5-amine

The mixture of tert-butyl N-[7-(2-pyrrolidin-1-ylethoxy)benzofuran-5-yl]carbamate (277 mg, 799.61 μmol) in DCM (3 mL) and TFA (3 mL) was stirred at 20° C. for 2 h. LCMS showed the reaction was complete. The reaction mixture was filtered and concentrated under reduced pressure to give a crude product. The residue was purified by prep-HPLC (TFA condition; column: Phenomenex luna C18 100*40 mm*5 um; mobile phase: [water(0.1% TFA)-ACN]; B %: 1%-15%,8 min). 7-(2-pyrrolidin-1-ylethoxy)benzofuran-5-amine (250 mg, crude) was obtained as yellow oil.

### Step 8. 7-(2-pyrrolidin-1-ylethoxy)-2,3-dihydrobenzofuran-5-amine

To a mixture of 7-(2-pyrrolidin-1-ylethoxy)benzofuran-5-amine (240 mg, 974.40 mol) in MeOH (30 mL) was added Pd/C (240 mg, 10% purity), then the mixture was stirred at 30° C. for 12 hrs under H₂ (50 Psi) atmosphere. LCMS showed the reaction was complete. The reaction mixture was filtered and concentrated under reduced pressure to give 7-(2-pyrrolidin-1-ylethoxy)-2,3-dihydrobenzofuran-5-amine (240 mg, crude) as brown oil.

### Step 9. N4,6-dimethyl-N2-[7-(2-pyrrolidin-1-ylethoxy)-2,3-dihydrobenzofuran-5-yl]-pyrimidine-2,4-diamine

A mixture of 7-(2-pyrrolidin-1-ylethoxy)-2,3-dihydrobenzofuran-5-amine (75.63 mg, 304.57 μmol), 2-chloro-N,6-dimethyl-pyrimidin-4-amine (40 mg, 253.81 μmol), TFA (2.89 mg, 25.38 μmol, 1.96 μL) in i-PrOH (2.00 mL) and then the mixture was stirred at 130° C. for 1 h under microwave heating. LCMS showed the reaction was complete. The reaction mixture was filtered and concentrated under reduced pressure to give a crude product. The residue was purified by prep-HPLC (TFA condition; column: Phenomenex luna C18 100*40 mm*5 um; mobile phase: [water(0.1% TFA)-ACN]; B %: 1%-27%,8 min).N4,6-dimethyl-N2-[7-(2-pyrrolidin-1-ylethoxy)-2,3-dihydrobenzofuran-5-yl]pyrimidine-2,4-diamine (4.8 mg, 9.95 μmol, 3.92% yield, TFA, purity: 100%) was obtained as a yellow solid.

¹H NMR (400 MHz, METHANOL-d₄) δ ppm 7.16 (s, 1H), 7.09 (br s, 1H), 5.96 (s, 1H), 4.67 (t, J=8.7 Hz, 2H), 4.43-4.34 (m, 2H), 3.78 (br s, 2H), 3.64 (br s, 2H), 3.30-3.14 (m, 4H), 2.97 (s, 3H), 2.28 (s, 3H), 2.20 (br s, 2H), 2.06 (br s, 2H).

### EXAMPLE 10. Synthesis of Compound 113

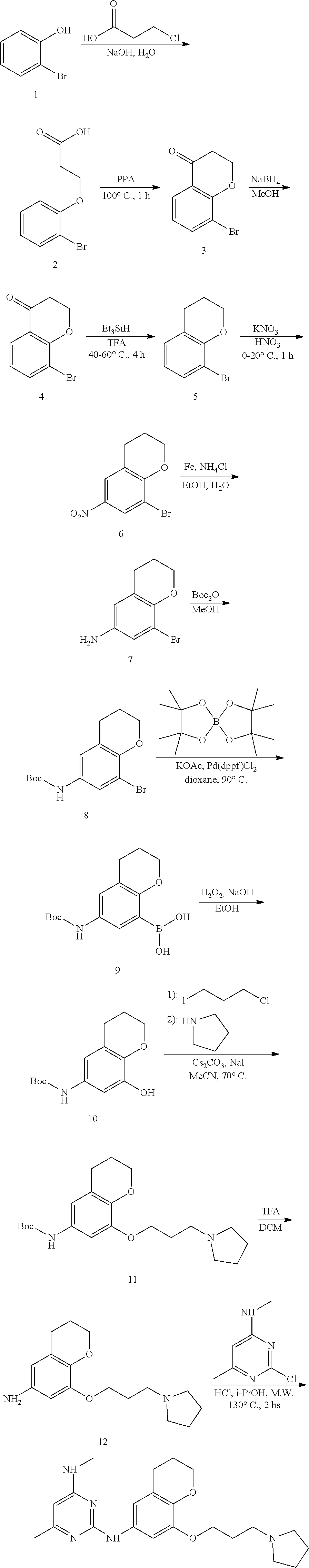

### Step 1. 3-(2-bromophenoxy)propanoic acid

To a solution of sodium hydroxide (5.55 g, 138.72 mmol, 2.60 mL) in H₂O (28.9 mL) was slowly added 2-bromophenol (10 g, 57.80 mmol, 6.70 mL) and 3-chloropropanoic acid (6.27 g, 57.80 mmol, 4.94 mL), then the mixture was stirred at 20° C. for 12 hrs. TLC (Petroleum ether/Ethyl acetate=3:1) showed the starting material (R1) remained and new spot were not formed. The reaction was stirred at 80° C. for 12 hrs. LCMS showed starting material remained and desired mass was detected. The reaction was stirred at 100° C. for 12 hrs. LCMS showed starting material remained and desired mass was detected. The reaction was filtered and concentrated under reduced pressure to give a residue. The crude product was purified by reversed-phase HPLC (column: C18 20-35 um 100A 800 g; mobile phase: [water-ACN]; B %: 5%-35% @ 120 mL/min) to give 3-(2-bromophenoxy)propanoic acid (7 g, crude) as yellow oil.

### Step 2. 8-bromochroman-4-one

A mixture of 3-(2-bromophenoxy)propanoic acid (6.5 g, 26.52 mmol) in PPA (70 mL) was stirred at 100° C. for 2 hrs. TLC (Petroleum ether/Ethyl acetate=1:1) showed the starting material (R1) was consumed and new spots were formed. The reaction mixture was quenched by water (1000 mL) and the mixture was acidified by Na₂CO₃ till pH=8, extracted with EtOAc (500 mL*3). The organic layer was dried over Na₂SO₄, concentrated to give 8-bromochroman-4-one (4.2 g, crude) as yellow gum.

### Step 3. 8-bromochroman-4-ol

To a solution of 8-bromochroman-4-one (4 g, 17.62 mmol) in MeOH (40 mL) was added sodium; boranuide (733.14 mg, 19.38 mmol, 682.63 μL), then the mixture was stirred at 20° C. for 1 hr. TLC (Petroleum ether/Ethyl acetate=1:1, Rf=0.5) showed the starting material (R1) was consumed and new spot was formed. The reaction mixture was concentrated to remover MeOH, The residue was added to water (50 mL). The mixture was extracted with EtOAc (100 mL*3), the organic layer was dried over Na₂SO₄, concentrated to give 8-bromochroman-4-ol (4 g, crude) as yellow oil.

### Step 4. 8-bromochroman

To a solution of 8-bromochroman-4-ol (4 g, 17.46 mmol) in TFA (40 mL) was added triethylsilane (4.47 g, 38.42 mmol, 6.14 mL), then the mixture was stirred at 62.5° C. for 12 hr. TLC (Petroleum ether/Ethyl acetate=2:1, Rf=0.5) showed the starting material (R1) was consumed and new spot was formed. The reaction mixture was quenched by water (100 mL), extracted with EtOAc (50 mL*3). The organic layer was dried over Na₂SO₄, concentrated to give 8-bromochromane (4 g, crude) as a black solid.

### Step 5. 8-bromo-6-nitrochroman

Potassium nitrate (1.05 g, 10.35 mmol, 496.10 μL) was added to a stirred solution of 8-bromochromane (1.47 g, 6.90 mmol) in H₂SO₄ (20 mL) at 5° C. Then the mixture was stirred at ° C. for 1 h. TLC (Petroleum ether:Ethyl acetate=2:1, Rf=0.5) showed the starting material was consumed and a new spot was formed. The reaction was added to crushed ice and extracted with chloroform (30 mL*3), and then the organic extracts were washed with brine (30 mL), dried over Na₂SO₄, and then the organic phase was concentrated in vacuum to give 8-bromo-6-nitro-chromane (1.64 g, crude) as a yellow solid.

### Step 6. 8-bromochroman-6-amine

To a mixture of 8-bromo-6-nitro-chromane (700 mg, 2.71 mmol) in EtOH (2 mL), H₂O (8 mL) THF (2 mL) was added ammonia hydrochloride (725.47 mg, 13.56 mmol) and Iron (605.91 mg, 10.85 mmol, 77.09 μL). Then the mixture was stirred at 70° C. for 3 hrs. LCMS showed the reaction was complete mostly and the desired ms was detected. The reaction was filtered and concentrated under reduced pressure to give 8-bromochroman-6-amine (1.43 g, crude) as a red solid.

### Step 7. tert-butyl (8-bromochroman-6-yl)carbamate

To a mixture of 8-bromochroman-6-amine (1.43 g, 6.27 mmol) in MeOH (15 mL) was added tert-butoxycarbonyl tert-butyl carbonate (4.11 g, 18.81 mmol, 4.32 mL), then the mixture was stirred at 70° C. for 12 hrs. LCMS showed the reaction was complete and the desired ms was detected. The reaction was concentrated under reduced pressure to give a residue. The residue was purified by column chromatography (SiO₂, Petroleum ether/Ethyl acetate=1/0 to 1/1) to give tert-butyl N-(8-bromochroman-6-yl)carbamate (780 mg, crude) as a yellow solid.

### Step 8. (6-((tert-butoxycarbonyl)amino)chroman-8-yl)boronic acid

To a mixture of 4,4,5,5-tetramethyl-2-(4,4,5,5-tetramethyl-1,3,2-dioxaborolan-2-yl)-1,3,2-dioxaborolane (185.69 mg, 731.26 μmol) tert-butyl N-(8-bromochroman-6-yl)carbamate (200 mg, 609.38 μmol) in dioxane (4 mL) was added potassium acetate (179.42 mg, 1.83 mmol, 114.28 μL) and cyclopentyl(diphenyl)phosphane;dichloromethane dichloropalladium;iron (99.53 mg, 121.88 μmol), then the reaction was stirred at 100° C. for 12 hrs under N2. LCMS showed the reaction was complete mostly and the desired ms was detected. The reaction was concentrated under reduced pressure to give [6-(tert-butoxycarbonylamino)chroman-8-yl]boronic acid (200 mg, crude) as a black oil.

### Step 9. tert-butyl (8-hydroxychroman-6-yl)carbamate

To a mixture of [6-(tert-butoxycarbonylamino)chroman-8-yl]boronic acid (178.62 g, 609.37 mmol) in EtOH (3 mL) was added hydrogen peroxide (125.62 g, 1.22 mol, 114.20 mL, 33% purity) at 0° C., then the reaction was stirred at 20° C. for 2 hrs. LCMS showed the reaction was complete and the desired ms was detected. The reaction was concentrated under reduced pressure to give a residue. The residue was purified by column chromatography (SiO₂, Petroleum ether/Ethyl acetate=1/0 to 5/1) to give tert-butyl N-(8-hydroxychroman-6-yl)carbamate (95 mg, crude) as a yellow solid.

### Step 10. tert-butyl (8-(3-(pyrrolidin-1-yl)propoxy)chroman-6-yl)carbamate

A mixture of tert-butyl N-(8-hydroxychroman-6-yl)carbamate (60 mg, 226.16 mol) and 1-chloro-3-iodo-propane (50.86 mg, 248.77 μmol, 26.71 μL) in MeCN (2 mL) was added dicesium carbonate (221.06 mg, 678.47 μmol), then the reaction was stirred at 50° C. for 1 h. LCMS showed the reaction was complete and the desired ms was detected. The mixture was used for the next step directly without workup. tert-butyl N-[8-(3-chloropropoxy)chroman-6-yl]carbamate (77.31 mg, crude) in MeCN (2 mL) was used for next step directly.

To a mixture of tert-butyl N-[8-(3-chloropropoxy)chroman-6-yl]carbamate (77 mg, 225.26 μmol) in MeCN (895.37 μL) was added NaI (40.52 mg, 270.31 μmol, 11.05 μL) and pyrrolidine (80.10 mg, 1.13 mmol, 93.58 μL), then the mixture was stirred at 80° C. for 12 hrs. LCMS showed the reaction was complete mostly and the desired ms was detected. The reaction was concentrated under reduced pressure to give tert-butyl N-[8-(3-pyrrolidin-1-ylpropoxy)chroman-6-yl]carbamate (190 mg, crude).

### Step 11. 8-(3-(pyrrolidin-1-yl)propoxy)chroman-6-amine

To a mixture of tert-butyl N-[8-(3-pyrrolidin-1-ylpropoxy)chroman-6-yl]carbamate (190 mg, 504.66 μmol) in DCM (2 mL) was added TFA (0.75 g, 6.58 mmol, 506.76 μL), then the mixture was stirred at 20° C. for 1 h. LCMS showed the reaction was complete mostly and the desired ms was detected. The reaction was concentrated under reduced pressure to give 8-(3-pyrrolidin-1-ylpropoxy)chroman-6-amine (100 mg, crude) as a black oil.

### Step 12. N4,6-dimethyl-N2-(8-(3-(pyrrolidin-1-yl)propoxy)chroman-6-yl)pyrimidine-2,4-diamine

To the mixture of tert-butyl N-[8-(3-pyrrolidin-1-ylpropoxy)chroman-6-yl]carbamate (40 mg, 106.24 μmol) and 2-chloro-N,6-dimethyl-pyrimidin-4-amine (16.74 mg, 106.24 μmol) in i-PrOH (3 mL) was added HCl (12 M, 8.85 μL), then the mixture was stirred at 120° C. in the microwave for 1 h. LCMS showed the reaction was complete mostly and the desired ms was detected. The reaction was filtered and concentrated under reduced pressure to give a residue. The residue was purified by prep-HPLC (TFA condition:column: Phenomenex luna C18 100*40 mm*5 um; mobile phase:water(0.1% TFA)-ACN]; B %: 1%-35%, 8 min) to give N4,6-dimethyl-N2-[8-(3-pyrrolidin-1-ylpropoxy)chroman-6-yl]pyrimidine-2,4-diamine (8.7 mg, 17.04 μmol, 16.04% yield, TFA) as a pale white solid.

¹H NMR (400 MHz, METHANOL-d₄) 6=7.14-7.08 (m, 1H), 6.98-6.94 (m, 1H), 5.97-5.93 (m, 1H), 4.26-4.21 (m, 2H), 4.17 (t, J=5.3 Hz, 2H), 3.88-3.74 (m, 2H), 3.49-3.43 (m, 2H), 3.21-3.07 (m, 2H), 2.99 (s, 3H), 2.81 (t, J=6.4 Hz, 2H), 2.28 (s, 3H), 2.20 (br s, 4H), 2.10-2.00 (m, 4H)

### EXAMPLE 11. Synthesis of Compound 114

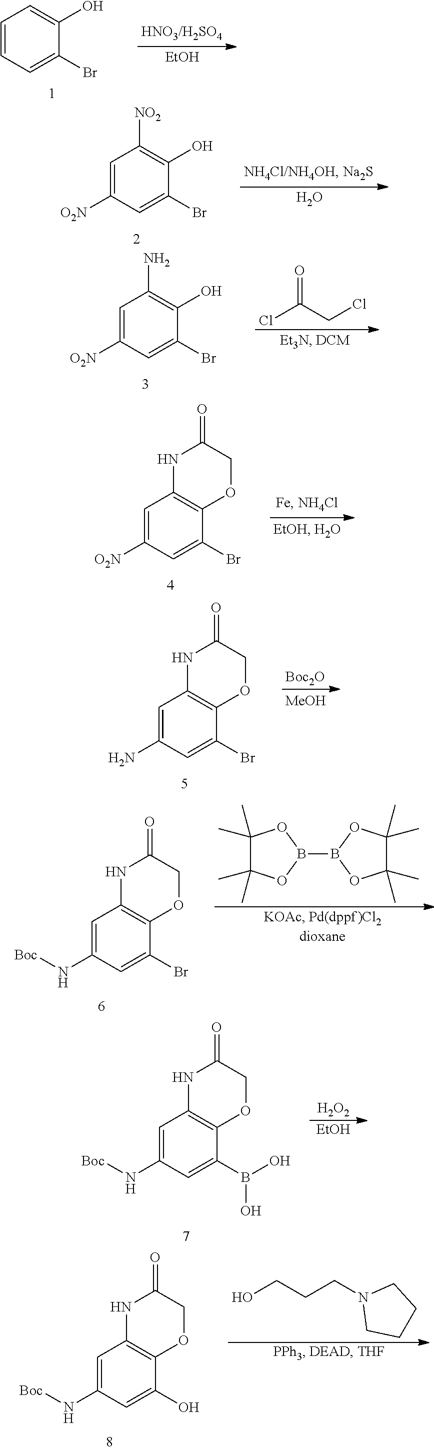

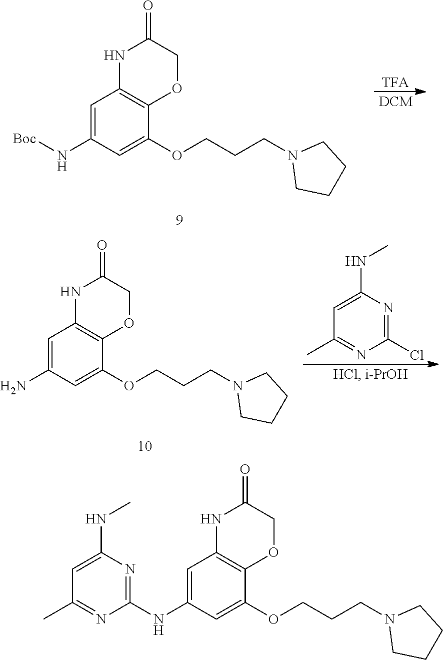

### Step 1. 2-bromo-4,6-dinitro-phenol

2-Bromophenol (10 g, 57.80 mmol, 6.70 mL) was dissolved in EtOH (50 mL) and H₂SO₄ (100 mL), and then to the mixture was added HNO₃ (30.00 g, 476.09 mmol, 20 mL) slowly, and then the mixture was stirred at 25° C. for 12 h. TLC (petroleum ether:ethyl acetate=0:1, Rf=0.5) indicated starting material was consumed completely, and one major new spot was detected. The mixture was added to H₂O (500 mL) at 5° C., and then the suspension was filtered, and washed with H₂O (50 mL*2), and the filtered cake was the desired compound, and the filtrate was quenched by sat. 2M NaOH to pH=7 at 25° C. slowly. 2-bromo-4,6-dinitro-phenol (5 g, crude) was obtained as a yellow solid.

### Step 2. 2-amino-6-bromo-4-nitro-phenol

NH₄Cl (10.00 g, 186.95 mmol) and NH₄OH (2.67 g, 19.01 mmol, 2 mL, 25% purity) were added to a solution of 2-bromo-4,6-dinitro-phenol (5 g, 19.01 mmol) in H₂O (50 mL). The mixture was heated to 80° C. Sodiosulfanyl sodium nonahydrate (5.50 g, 22.90 mmol) was added. After addition, reaction was heated for 2 h at 80° C. LCMS showed starting material was consumed completely and desired MS was detected. TLC (petroleum ether:ethyl acetate=3:1, Rf=0.1) indicated starting material was consumed completely, and one major new spot was detected. The mixture was diluted with H₂O (200 mL), and then to the mixture was added AcOH to pH=2 at 10° C., and then extracted with EtOAc (100 mL*3), and then the organic phase was concentrated in vacuum to give 2-amino-6-bromo-4-nitro-phenol (2.5 g, crude) as a black solid.

### Step 3. 8-bromo-6-nitro-4H-1,4-benzoxazin-3-one

2-Amino-6-bromo-4-nitro-phenol (2 g, 8.58 mmol) was dissolved in DCM (30 mL) and then to the mixture was added TEA (2.61 g, 25.75 mmol, 3.59 mL) 2-chloroacetyl chloride (1.16 g, 10.30 mmol, 820.93 μL) and then the mixture was stirred at 25° C. for 12 hr. LCMS showed starting material was consumed completely and a main peak was detected. TLC (petroleum ether:ethyl acetate=5:1, Rf=0.1) indicated starting material was consumed completely, and one major new spot was detected. The reaction was diluted by H₂O (100 mL), and then extracted with DCM (20 mL*2), the organic phase was concentrated in vacuum. 8-bromo-6-nitro-4H-1,4-benzoxazin-3-one (2.3 g, crude) was obtained as a yellow oil.

### Step 4. 6-amino-8-bromo-4H-1,4-benzoxazin-3-one

8-Bromo-6-nitro-4H-1,4-benzoxazin-3-one (2.3 g, 8.42 mmol) was dissolved in H₂O (10 mL) EtOH (40 mL), and then to the mixture was added Fe (4.70 g, 84.24 mmol) NH₄Cl (4.51 g, 84.24 mmol), and then the mixture was stirred at 80° C. for 1 h. LCMS showed starting material was consumed completely and desired MS was detected. The reaction was cooled to 50° C., and the filtered, the filter cake was washed with MeOH (40 mL*2). The filtrate was concentrated in vacuum at 50° C. 6-amino-8-bromo-4H-1,4-benzoxazin-3-one (2 g, crude) was obtained as a black solid.

### Step 5. tert-butyl N-(8-bromo-3-oxo-4H-1,4-benzoxazin-6-yl)carbamate

6-Amino-8-bromo-4H-1,4-benzoxazin-3-one (2.05 g, 8.43 mmol) was dissolved in MeOH (50 mL), and then to the mixture was added tert-butoxycarbonyl tert-butyl carbonate (5.52 g, 25.30 mmol, 5.81 mL), and then the mixture was stirred at 60° C. for 12 h. LCMS showed starting material was consumed completely and desired MS was detected. The mixture was concentrated in vacuum, and the residue was purified by flash chromatography (silica gel, petroleum ether/ethyl acetate=2/1 to 1/1 get the spot, 0/1). Tert-butyl N-(8-bromo-3-oxo-4H-1,4-benzoxazin-6-yl)carbamate (2 g, crude) was obtained as a yellow solid.

### Step 6. [6-(tert-butoxycarbonylamino)-3-oxo-4H-1,4-benzoxazin-8-yl]boronic acid

Tert-butyl N-(8-bromo-3-oxo-4H-1,4-benzoxazin-6-yl)carbamate (1.8 g, 5.25 mmol) was dissolved in dioxane (30 mL) and the to the mixture was added 4,4,5,5-tetramethyl-2-(4,4,5,5-tetramethyl-1,3,2-dioxaborolan-2-yl)-1,3,2-dioxaborolane (1.60 g, 6.29 mmol), KOAc (2.96 g, 10.49 mmol) cyclopentyl(diphenyl)phosphane dichloropalladium iron (383.79 mg, 524.52 mol), and then the mixture was stirred at 80° C. for 12 h under N2. LCMS showed starting material was consumed completely and desired MS was detected. The mixture was concentrated in vacuum to give [6-(tert-butoxycarbonylamino)-3-oxo-4H-1,4-benzoxazin-8-yl]boronic acid (1.62 g, crude) was obtained as a black solid.

### Step 7. tert-butyl N-(8-hydroxy-3-oxo-4H-1,4-benzoxazin-6-yl)carbamate

[6-(Tert-butoxycarbonylamino)-3-oxo-4H-1,4-benzoxazin-8-yl]boronic acid (1.6 g, 5.19 mmol) was dissolved in EtOH (30 mL), and to the mixture was added H₂O₂ (1.18 g, 10.39 mmol, 30% purity) at 0° C., and then the mixture was stirred at 25° C. for 2 h. LCMS showed starting material was consumed completely and desired MS was detected. The mixture was diluted with H₂O (60 mL), and then the mixture was quenched by sat. aq. Na₂SO₃ (50 mL), and then the mixture was extracted with EtOAc (30 mL*3). The organic phase was concentrated in vacuum. The residue was purified by flash chromatography (silica gel, petroleum ether/ethyl acetate=50/1 to 1/1 get the spot, 0/1). tert-butyl N-(8-hydroxy-3-oxo-4H-1,4-benzoxazin-6-yl)carbamate (1.3 g, crude) was obtained as a yellow solid.

### Step 8. tert-butyl N-[3-oxo-8-(3-pyrrolidin-1-ylpropoxy)-4H-1,4-benzoxazin-6-yl]carbamate

Tert-butyl N-(8-hydroxy-3-oxo-4H-1,4-benzoxazin-6-yl)carbamate (220 mg, 784.94 mol) was dissolved in THF (4 mL), and then to the mixture was added 3-pyrrolidin-1-ylpropan-1-ol (121.70 mg, 941.93 μmol), PPh₃ (308.82 mg, 1.18 mmol), and then to the mixture was added ethyl (NE)-N-ethoxycarbonyliminocarbamate (238.08 mg, 1.37 mmol, 231.82 μL) under N2 at 0° C., then the mixture was stirred at 20° C. for 12 hrs under N2. LCMS showed the reaction was complete and the desired ms was detected. The reaction was filtered and concentrated under reduced pressure to give a residue. The crude product was purified by prep-HPLC(TFA condition:column: Phenomenex luna C18 100*40 mm*5 um; mobile phase:water(0.1% TFA)-ACN; B %: 5%-38%, 8 min) to give tert-butyl N-[3-oxo-8-(3-pyrrolidin-1-ylpropoxy)-4H-1,4-benzoxazin-6-yl]carbamate (120 mg, crude) as white solid.

### Step 9. 6-amino-8-(3-pyrrolidin-1-ylpropoxy)-4H-1,4-benzoxazin-3-one

To a solution of tert-butyl N-[3-oxo-8-(3-pyrrolidin-1-ylpropoxy)-4H-1,4-benzoxazin-6-yl]carbamate (110 mg, 281.00 mol) in DCM (2 mL) was added 2,2,2-trifluoroacetic acid (296.00 mg, 2.60 mmol, 0.2 mL), then the mixture was stirred at 20° C. for 4 hrs. LCMS showed the reaction was complete and the desired ms was detected. The reaction was concentrated under reduced pressure to give 6-amino-8-(3-pyrrolidin-1-ylpropoxy)-4H-1,4-benzoxazin-3-one (80 mg, crude) as white solid.

### Step 10. 6-[[4-methyl-6-(methylamino)pyrimidin-2-yl]amino]-8-(3-pyrrolidin-1-ylpropoxy)-4H-1,4-benzoxazin-3-one

To the mixture of 6-amino-8-(3-pyrrolidin-1-ylpropoxy)-4H-1,4-benzoxazin-3-one (20 mg, 68.65 mol) and 2-chloro-N,6-dimethyl-pyrimidin-4-amine (10.82 mg, 68.65 μmol) in i-PrOH (3 mL) was added HCl (12 M, 0.04 mL), then the mixture was stirred at 120° C. in the microwave for 0.5 hrs. LCMS showed the reaction was complete and the desired ms was detected. The reaction was filtered and concentrated under reduced pressure to give a residue. The crude product was purified by prep-HPLC(TFA condition:Phenomenex luna C18 100*40 mm*5 um; mobile phase:water(0.1% TFA)-ACN; B %: 1%-26%, 8 min) to give 6-[[4-methyl-6-(methylamino) pyrimidin-2-yl]amino]-8-(3-pyrrolidin-1-ylpropoxy)-4H-1,4-benzoxazin-3-one (100.2 mg, 190.68 μmol, 69.61% yield, TFA) as white solid.

¹H NMR (400 MHz, METHANOL-d₄) 6=7.09-6.85 (m, 2H), 6.05-5.93 (m, 1H), 4.63-4.56 (m, 2H), 4.23-4.16 (m, 2H), 3.84-3.75 (m, 2H), 3.49-3.42 (m, 2H), 3.17-3.10 (m, 2H), 3.06-2.97 (m, 3H), 2.33-2.24 (m, 5H), 2.23-2.16 (m, 2H), 2.09-2.01 (m, 2H). MS (ESI): m/z=413.1 [M+H]

### EXAMPLE 12. Synthesis of Compound 115

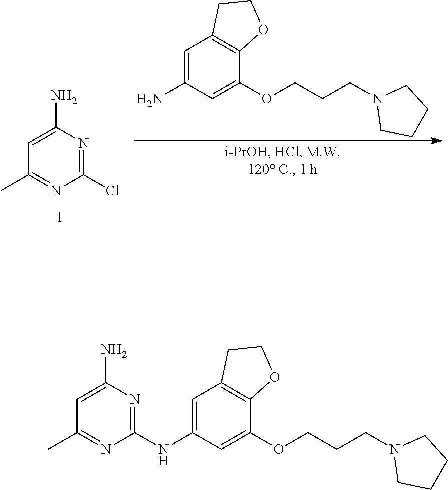

### 6-methyl-N2-[7-(3-pyrrolidin-1-ylpropoxy)-2,3-dihydrobenzofuran-5-yl]pyrimidine-2,4-diamine

To the mixture of 2-chloro-6-methyl-pyrimidin-4-amine (40 mg, 278.60 μmol) and 7-(3-pyrrolidin-1-ylpropoxy)-2,3-dihydrobenzofuran-5-amine (73.09 mg, 278.60 μmol) in i-PrOH (3 mL) was added HCl (12 M, 0.1 mL), then the mixture was stirred at 120° C. in the microwave for 1 hrs. LCMS showed the starting material remained and the desired ms was detected. The reaction was filtered and concentrated under reduced pressure to give a residue. The crude product was purified by prep-HPLC (TFA condition:column: Phenomenex luna C18 100*40 mm*5 um; mobile phase:water(0.1% TFA)-ACN; B %: 1%-20%, 8 min) to give 6-methyl-N2-[7-(3-pyrrolidin-1-ylpropoxy)-2,3-dihydrobenzofuran-5-yl]pyrimidine-2,4-diamine (41.2 mg, 85.39 mol, 30.65% yield, TFA) as pale yellow solid.

¹H NMR (400 MHz, METHANOL-d₄) 6=7.12 (s, 1H), 7.00-6.95 (m, 1H), 5.99-5.97 (m, 1H), 4.65-4.58 (m, 2H), 4.20 (br d, J=11.1 Hz, 2H), 3.80-3.70 (m, 2H), 3.46-3.41 (m, 2H), 3.28-3.21 (m, 2H), 3.17-3.06 (m, 2H), 2.32-2.28 (m, 3H), 2.14 (br s, 4H), 2.10-2.00 (m, 2H). MS (ESI): m/z=370.1 [M+H]

### EXAMPLE 13. Synthesis of Compound 116

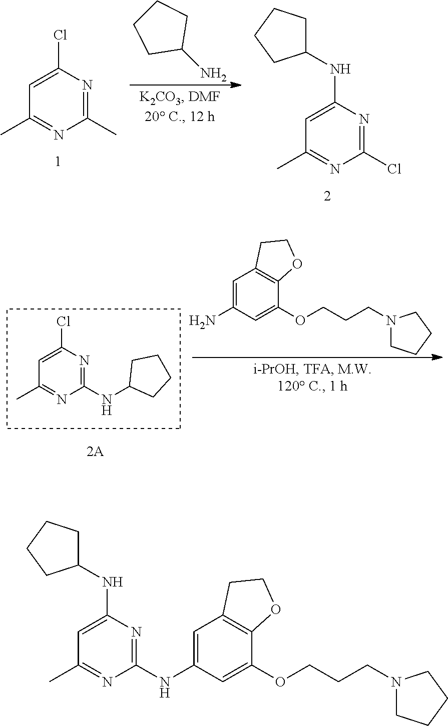

### Step 1. 2-chloro-N-cyclopentyl-6-methyl-pyrimidin-4-amine

To a mixture of 2,4-dichloro-6-methyl-pyrimidine (1 g, 6.13 mmol) and cyclopentanamine (574.60 mg, 6.75 mmol, 665.81 μL) in DCM (20 mL) was added K₂CO₃ (1.70 g, 12.27 mmol) at 20° C., then the mixture was stirred at 20° C. for 12 hrs. LCMS showed the reaction was complete and the desired ms was detected. The reaction mixture was added to water (20 mL), extracted with EtOAc (25 mL*3). The organic layer was dried over Na₂SO₄, concentrated to give the crude product. The residue was purified by column chromatography (SiO₂, Petroleum ether/Ethyl acetate=1/0 to 0/1) to give 2-chloro-N-cyclopentyl-6-methyl-pyrimidin-4-amine (950 mg, crude) as yellow oil and 4-chloro-N-cyclopentyl-6-methyl-pyrimidin-2-amine (600 mg, crude) as yellow oil.

### Step 2. N4-cyclopentyl-6-methyl-N2-[7-(3-pyrrolidin-1-ylpropoxy)-2,3-dihydrobenzofuran-5-yl]-pyrimidine-2,4-diamine

To the mixture of 2-chloro-N-cyclopentyl-6-methyl-pyrimidin-4-amine (40 mg, 188.95 μmol) and 7-(3-pyrrolidin-1-ylpropoxy)-2,3-dihydrobenzofuran-5-amine (49.57 mg, 188.95 μmol) in i-PrOH (10 mL) was added HCl (12 M, 0.1 mL), then the mixture was stirred at 120° C. in the microwave for 1 hrs. LCMS showed the starting material remained and the desired ms was detected. The reaction was filtered and concentrated under reduced pressure to give a residue. The crude product was purified by prep-HPLC (TFA condition:column:Phenomenex luna C18 100*40 mm*5 um; mobile phase:water(0.1% TFA)-ACN; B %: 10%-40%, 8 min) to give N4-cyclopentyl-6-methyl-N2-[7-(3-pyrrolidin-1-ylpropoxy)-2,3-dihydrobenzofuran-5-yl]pyrimidine-2,4-diamine (18.2 mg, 33.06 μmol, 17.49% yield, TFA) as gray solid.

¹H NMR (400 MHz, METHANOL-d₄) 6=7.19 (br s, 1H), 7.00-6.96 (m, 1H), 5.95-5.90 (m, 1H), 4.66-4.60 (m, 2H), 4.35-4.26 (m, 1H), 4.21-4.17 (m, 2H), 3.81-3.69 (m, 2H), 3.46-3.41 (m, 2H), 3.27-3.22 (m, 2H), 3.18-3.07 (m, 2H), 2.30-2.25 (m, 3H), 2.24-2.14 (m, 4H), 2.10-1.98 (m, 4H), 1.82-1.73 (m, 2H), 1.66-1.53 (m, 4H). MS (ESI): m/z=438.2 [M+H].

### EXAMPLE 14. Synthesis of Compound 117

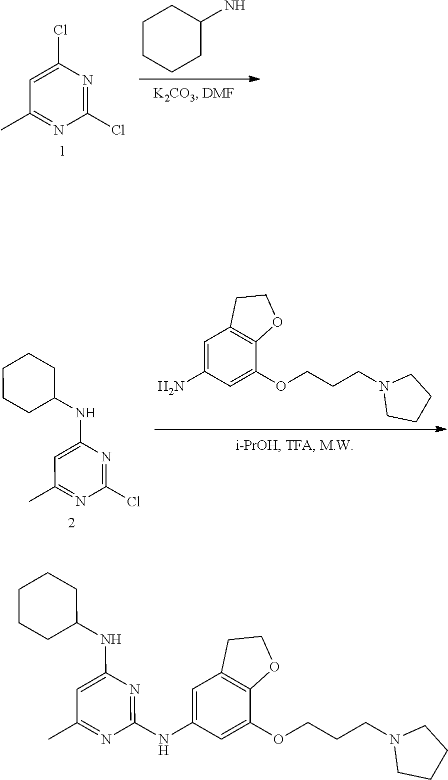

### Step 1. 2-chloro-N-cyclohexyl-6-methyl-pyrimidin-4-amine

To a mixture of 2,4-dichloro-6-methyl-pyrimidine (1 g, 6.13 mmol, 1 eq.) and cyclohexanamine (669.25 mg, 6.75 mmol, 771.03 μL, 1.1 eq.) in DMF (20 mL) was added tripotassium carbonate (1.70 g, 12.27 mmol, 740.49 μL, 2 eq.) at 25° C., then the mixture was stirred at 25° C. for 12 hrs. LCMS showed the reaction was complete mostly and the desired ms was detected. The reaction mixture was added to water (30 mL), extracted with EtOAc (15 mL*3). The organic layer was dried over Na₂SO₄, concentrated to give the crude product. The crude product was purified by column chromatography (SiO₂, Petroleum ether/Ethyl acetate=20/1 to 3/1) to give 2-chloro-N-cyclohexyl-6-methyl-pyrimidin-4-amine (1.22 g, crude) as a yellow oil.

### Step 2. N4-cyclohexyl-6-methyl-N2-[7-(3-pyrrolidin-1-yl]propoxy)-2,3-dihydrobenzofuran-5-yl]pyrimidine-2,4-diamine

To a mixture of 2-chloro-N-cyclohexyl-6-methyl-pyrimidin-4-amine (40 mg, 177.21 μmol, 1 eq.) and 7-(3-pyrrolidin-1-ylpropoxy)-2,3-dihydrobenzofuran-5-amine (46.49 mg, 177.21 μmol, 1 eq.) in i-PrOH (10 mL) was added HCl (12 M, 14.77 μL, 1 eq.), then the mixture was stirred at 120° C. in the microwave for 1 h. LCMS showed the starting material was remained and the desired ms was detected. The reaction was filtered and concentrated under reduced pressure to give a residue. The crude product was purified by prep-HPLC(TFA condition:column:Phenomenex luna C18 100*40 mm*5 um; mobile phase:water(0.1% TFA)-ACN]; B %: 10%-45%, 8 min) to give N4-cyclohexyl-6-methyl-N2-[7-(3-pyrrolidin-1-ylpropoxy)-2,3-dihydrobenzofuran-5-yl]pyrimidine-2,4-diamine (12.6 mg, 22.32 μmol, 12.59% yield, TFA) as a white solid.

¹H NMR (400 MHz, METHANOL-d₄) δ ppm 7.20 (br d, J=6.9 Hz, 1H), 6.90 (s, 1H), 5.91 (s, 1H), 4.62 (br d, J=8.8 Hz, 2H), 4.24-4.14 (m, 3H), 3.91-3.68 (m, 4H), 3.47-3.38 (m, 3H), 3.28-3.22 (m, 3H), 3.17-3.06 (m, 2H), 2.28-2.26 (m, 3H), 2.22-2.18 (m, 3H), 2.08-1.93 (m, 5H), 1.84-1.78 (m, 2H), 1.70-1.65 (m, 1H), 1.37-1.24 (m, 6H). MS (ESI): m/z=452.2 [M+H]⁺

### EXAMPLE 15. Synthesis of Compound 118

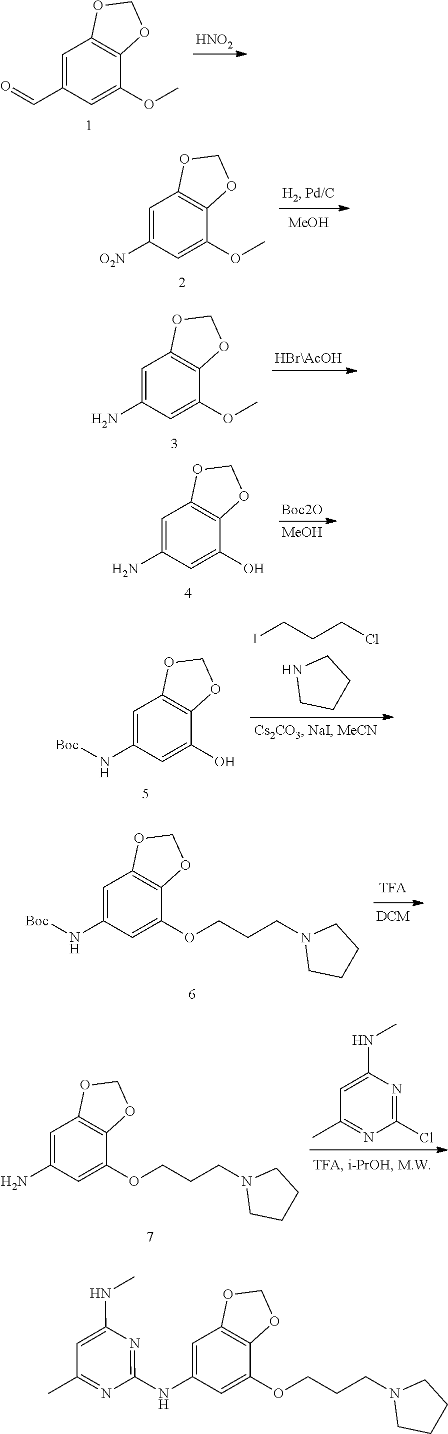

### Step 1. 4-methoxy-6-nitrobenzo[d][1,3dioxole

To a flask containing stirred nitric acid (15.81 g, 250.88 mmol, 6 mL) cooled to 0° C. was added 7-methoxy-1,3-benzodioxole-5-carbaldehyde (1 g, 5.55 mmol) portion wise. The reaction was stirred at 0° C. for 2 hrs. TLC (Petroleum ether:Ethyl acetate=4:1, Rf=0.45) showed the starting material was consumed and new spot was formed. The reaction mixture was quenched by water (50 mL), then the mixture was filtered to give 4-methoxy-6-nitro-1,3-benzodioxole (1.46 g, crude) a light yellow solid.

### Step 2. 7-methoxybenzo[d][1,3]dioxol-5-amine

A mixture of 4-methoxy-6-nitro-1,3-benzodioxole (360 mg, 1.83 mmol) in EtOAc (20 mL) was added Pd/C (300 mg, 10% purity) at 20° C., then the reaction was stirred at 20° C. for 12 hrs under H₂ (15 Psi). TLC (Petroleum ether:Ethyl acetate=2:1, Rf=0.4) showed the starting material (R1) was consumed and a new spot was formed. The reaction was filtered and concentrated under reduced pressure to give a residue. The residue was purified by prep-TLC (Petroleum ether/Ethyl acetate=2:1, Rf=0.4) to give 7-methoxy-1,3-benzodioxol-5-amine (220 mg, crude) as a light yellow oil.

### Step 3. 6-aminobenzo[d][1,3]dioxol-4-ol

To a mixture of 7-bromo-2,2-difluoro-1,3-benzodioxol-5-amine (36 mg, 142.85 mol) and 2-chloro-N,6-dimethyl-pyrimidin-4-amine (22.51 mg, 142.85 μmol) in i-PrOH (3 mL) was added HCl (12 M, 11.90 μL), then the mixture was stirred at 130° C. in the microwave for 1 hrs. LCMS showed the reaction was complete mostly and the desired ms was detected. The reaction was concentrated under reduced pressure to give N2-(7-bromo-2,2-difluoro-1,3-benzodioxol-5-yl)-N4,6-dimethyl-pyrimidine-2,4-diamine (60 mg, crude) as a light yellow solid.

### Step 4. tert-butyl (7-hydroxybenzo[d][1,3]dioxol-5-yl)carbamate

To a mixture of 6-amino-1,3-benzodioxol-4-ol (32.00 mg, 208.97 μmol) in MeOH (856.12 μL) was added tert-butoxycarbonyl tert-butyl carbonate (136.82 mg, 626.90 μmol, 143.87 μL), then the mixture was stirred at 60° C. for 12 hrs. The reaction was concentrated under reduced pressure to give a residue. The residue was used for the next step directly without workup. tert-butyl N-(7-hydroxy-1,3-benzodioxol-5-yl)carbamate (52.92 mg, crude) was obtained as a black solid.

### Step 5. tert-butyl (7-(3-(pyrrolidin-1-yl)propoxy)benzo[d][1,3]dioxol-5-yl)carbamate

A mixture of 1-chloro-3-iodo-propane (85.44 mg, 417.93 μmol, 44.87 μL) and tert-butyl N-(7-hydroxy-1,3-benzodioxol-5-yl)carbamate (52.92 mg, 208.96 μmol) in MeCN (1.98 mL) was added dicesium;carbonate (204.25 mg, 626.89 μmol), then the reaction was stirred at 50° C. for 1 h. The mixture was used for the next step directly without workup. tert-butyl N-[7-(3-chloropropoxy)-1,3-benzodioxol-5-yl]carbamate (68.91 mg, crude) in MeCN (2 mL) was used for next step directly.

To a mixture of tert-butyl N-[7-(3-chloropropoxy)-1,3-benzodioxol-5-yl]carbamate (68.91 mg, 208.96 μmol) in MeCN (1 mL) was added NaI (37.59 mg, 250.75 μmol, 10.25 μL) and pyrrolidine (74.31 mg, 1.04 mmol, 86.81 μL), then the mixture was stirred at 80° C. for 12 hrs. The reaction was concentrated under reduced pressure to give tert-butyl N-[7-(3-pyrrolidin-1-ylpropoxy)-1,3-benzodioxol-5-yl]carbamate (122 mg, crude) as a black oil.

### Step 6. 7-(3-(pyrrolidin-1-yl)propoxy)benzo[d][1,3]dioxol-5-amine

To a mixture of tert-butyl N-[7-(3-pyrrolidin-1-ylpropoxy)-1,3-benzodioxol-5-yl]carbamate (122 mg, 334.76 mol) in DCM (2.04 mL) was added 2,2,2-trifluoroacetic acid (725.25 mg, 6.36 mmol, 490.03 μL), then the mixture was stirred at 20° C. for 1 h. The reaction was filtered and concentrated under reduced pressure to give a residue. The crude product was purified by prep-HPLC, TFA condition:column: Phenomenex luna C18 100*40 mm*5 um; mobile phase:water(0.1% TFA)-ACN]; B %: 1%-18%, 8 min) to give 7-(3-pyrrolidin-1-ylpropoxy)-1,3-benzodioxol-5-amine (10 mg, crude) as a yellow oil.

### Step 7. N4,6-dimethyl-N2-(7-(3-(pyrrolidin-1-yl)propoxy)benzo[d][1,3]dioxol-5-yl)pyrimidine-2,4-diamine

To a mixture 2-chloro-N,6-dimethyl-pyrimidin-4-amine (11.93 mg, 75.67 μmol) and 7-(3-pyrrolidin-1-ylpropoxy)-1,3-benzodioxol-5-amine (20 mg, 75.67 μmol) in i-PrOH (2.97 mL) was added HCl (12 M, 63.06 μL), then the mixture was stirred at 120° C. in the microwave for 40 min. The reaction was concentrated under reduced pressure to give a residue. The crude product was purified by prep-HPLC TFA condition:column: Phenomenex luna C18 100*40 mm*5 um; mobile phase:water(0.1% TFA)-ACN]; B %: 1%-32%, 8 min) to give N4,6-dimethyl-N2-[7-(3-pyrrolidin-1-ylpropoxy)-1,3-benzodioxol-5-yl]pyrimidine-2,4-diamine (27.1 mg, 54.37 mol, 71.85% yield, TFA) as a pale yellow solid.

¹H NMR (400 MHz, METHANOL-d₄) 6=7.00 (d, J=1.5 Hz, 1H), 6.83-6.76 (m, 1H), 5.97-5.94 (m, 3H), 4.25 (t, J=5.7 Hz, 2H), 3.72 (br s, 2H), 3.44-3.39 (m, 2H), 3.19-3.07 (m, 2H), 2.99 (s, 3H), 2.30-2.27 (m, 3H), 2.25-2.15 (m, 4H), 2.08-1.98 (m, 2H). MS (ESI): m/z=386.1 [M+H]⁺

### EXAMPLE 16. Synthesis of Compound 119

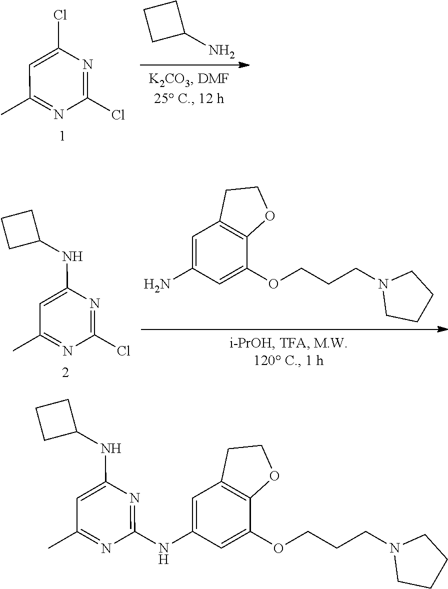

### Step 1. 2-chloro-N-cyclobutyl-6-methyl-pyrimidin-4-amine

To a mixture of 2,4-dichloro-6-methyl-pyrimidine (1 g, 6.13 mmol, 1 eq.) and cyclobutanamine (479.94 mg, 6.75 mmol, 576.16 μL, 1.1 eq.) in DMF (20 mL) was added K₂CO₃ (1.70 g, 12.27 mmol, 2 eq.) at 25° C., then the mixture was stirred at 25° C. for 12 hrs. LCMS showed the desired ms was detected. The reaction mixture was added to water (20 mL), extracted with EtOAc (10 mL*4). The organic layer was dried over Na₂SO₄, concentrated in vacuo. The residue was purified by flash chromatography (silica gel, petroleum ether/ethyl acetate=50/1 to 10/1 get the spot, 1/1). 2-chloro-N-cyclobutyl-6-methyl-pyrimidin-4-amine (800 mg, 4.05 mmol, 65.97% yield) was obtained as a colorless oil.

### Step 2. N4-cyclobutyl-6-methyl-N2-[7-(3-pyrrolidin-1-ylpropoxy)-2,3-dihydrobenzofuran-5-yl]pyrimidine-2,4-diamine

To a mixture of 2-chloro-N-cyclobutyl-6-methyl-pyrimidin-4-amine (40 mg, 202.36 μmol, 1 eq.) and 7-(3-pyrrolidin-1-ylpropoxy)-2,3-dihydrobenzofuran-5-amine (53.09 mg, 202.36 μmol, 1 eq.) in i-PrOH (3 mL) was added HCl (12 M, 16.86 μL, 1 eq.), then the mixture was stirred at 120° C. in the microwave for 1 h. LCMS showed the starting material was remained and the desired ms was detected. The reaction was filtered and concentrated under reduced pressure to give a residue. The crude product was purified by prep-HPLC(TFA condition:column:Phenomenex luna C18 100*40 mm*5 um; mobile phase:water(0.1% TFA)-ACN]; B %: 10%-40%,8 min) to give N4-cyclobutyl-6-methyl-N2-[7-(3-pyrrolidin-1-ylpropoxy)-2,3-dihydrobenzofuran-5-yl]pyrimidine-2,4-diamine (22.2 mg, 41.37 mol, 20.45% yield, TFA) as a white solid.

¹H NMR (400 MHz, METHANOL-d₄) δ ppm 7.15-7.09 (m, 1H), 7.02-6.97 (m, 1H), 5.94-5.87 (m, 1H), 4.67-4.60 (m, 2H), 4.53-4.41 (m, 1H), 4.24-4.18 (m, 2H), 3.81-3.70 (m, 2H), 3.47-3.42 (m, 2H), 3.29-3.23 (m, 2H), 3.19-3.07 (m, 2H), 2.37-2.31 (m, 2H), 2.27 (s, 3H), 2.25-2.14 (m, 4H), 2.11-1.99 (m, 4H), 1.86 (br s, 2H). MS (ESI): m/z=424.2 [M+H]⁺.

### EXAMPLE 17. Synthesis of Compounds 129

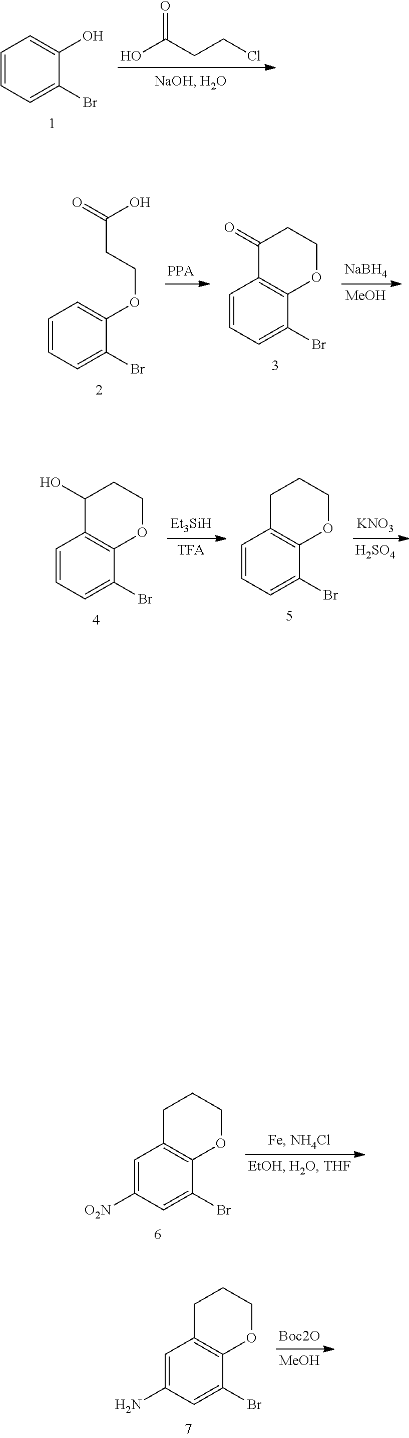

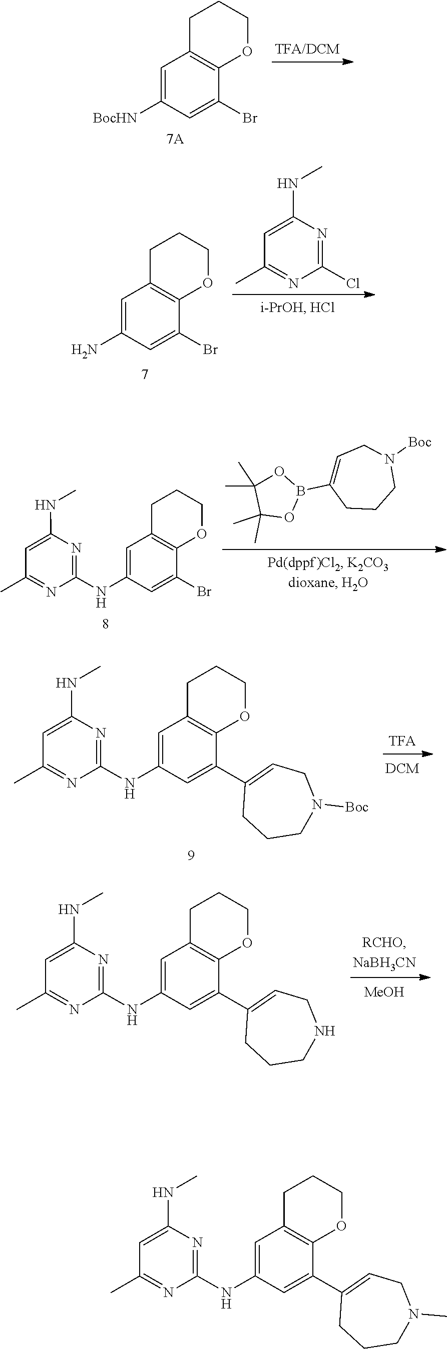

### Step 1. 3-(2-bromophenoxy) propanoic acid

To a solution of sodium; hydroxide (5.55 g, 138.72 mmol, 2.60 mL) in H₂O (28.9 mL) was slowly added 2-bromophenol (10 g, 57.80 mmol, 6.70 mL) and 3-chloropropanoic acid (6.27 g, 57.80 mmol, 4.94 mL), then the mixture was stirred at 20° C. for 36 h. LCMS showed desired mass was detected. The reaction was filtered and concentrated under reduced pressure to give a residue. The crude product was purified by reversed-phase HPLC (column: C18 20-35 um 100A 800 g; mobile phase: [water-ACN]; B %: 5%-35% @ 120 mL/min) to give 3-(2-bromophenoxy) propanoic acid (7 g, crude) as yellow oil.

### Step 2. 8-bromochroman-4-one

A mixture of 3-(2-bromophenoxy) propanoic acid (6.5 g, 26.52 mmol) in PPA (70 mL) was stirred at 100° C. for 2 h. TLC (Petroleum ether/Ethyl acetate=1:1) showed the starting material (R1) was consumed and new spots were formed. The reaction mixture was quenched by water (1000 mL) and the mixture was acidified by Na₂CO₃ till pH=8, extracted with EtOAc (500 mL×3). The organic layer was dried over Na₂SO₄, concentrated to give 8-bromochroman-4-one (4.2 g, crude) as yellow gum.

### Step 3. 8-bromochroman-4-ol

To a solution of 8-bromochroman-4-one (4 g, 17.62 mmol) in MeOH (40 mL) was added sodium boranuide (733.14 mg, 19.38 mmol, 682.63 μL), then the mixture was stirred at 20° C. for 1 hr. TLC (Petroleum ether/Ethyl acetate=1:1, Rf=0.5) showed the starting material (R1) was consumed and new spot was formed. The reaction mixture was concentrated to remover MeOH, The residue was added to water (50 mL). The mixture was extracted with EtOAc (100 mL×3), the organic layer was dried over Na₂SO₄, concentrated to give 8-bromochroman-4-ol (4 g, crude) as yellow oil.

### Step 4. 8-bromochromane

To a solution of 8-bromochroman-4-ol (4 g, 17.46 mmol) in TFA (40 mL) was added triethylsilane (4.47 g, 38.42 mmol, 6.14 mL), then the mixture was stirred at 62.5° C. for 12 h. TLC (Petroleum ether/Ethyl acetate=2:1, Rf=0.5) showed the starting material (R1) was consumed and new spot was formed. The reaction mixture was quenched by water (100 mL), extracted with EtOAc (50 mL×3). The organic layer was dried over Na₂SO₄, concentrated to give 8-bromochromane (4 g, crude) as black solid.

### Step 5. 8-bromo-6-nitro-chromane

Potassium nitrate (1.05 g, 10.35 mmol, 496.10 μL) was added to a stirred solution of 8-bromochromane (1.47 g, 6.90 mmol) in H₂SO₄ (20 mL) at 5° C. Then the mixture was stirred at 20° C. for 1 h. TLC (Petroleum ether:Ethyl acetate=2:1, Rf=0.5) showed the starting material was consumed and a new spot was formed. The reaction was added to crushed ice and extracted with chloroform (30 mL×3), and then the organic extracts were washed with brine (30 mL), dried over Na₂SO₄, and then the organic phase was concentrated in vacuo to give 8-bromo-6-nitro-chromane (1.64 g, crude) as a yellow solid.

### Step 6. 8-bromochroman-6-amine

To a mixture of 8-bromo-6-nitro-chromane (700 mg, 2.71 mmol) in EtOH (2 mL), H₂O (8 mL) THF (2 mL) was added ammonia hydrochloride (725.47 mg, 13.56 mmol) and Iron (605.91 mg, 10.85 mmol, 77.09 μL). Then the mixture was stirred at 70° C. for 3 h. LCMS showed the reaction was complete mostly and the desired MS was detected. The reaction was filtered and concentrated under reduced pressure to give 8-bromochroman-6-amine (1.43 g, crude) as a red solid.

### Step 7. tert-butyl N-(8-bromochroman-6-yl)carbamate

To a mixture of 8-bromochroman-6-amine (1.43 g, 6.27 mmol) in MeOH (15 mL) was added tert-butoxycarbonyl tert-butyl carbonate (4.11 g, 18.81 mmol, 4.32 mL), then the mixture was stirred at 70° C. for 12 h. LCMS showed the reaction was complete and the desired ms was detected. The reaction was concentrated under reduced pressure to give a residue. The residue was purified by column chromatography (SiO₂, Petroleum ether/Ethyl acetate=1/0 to 1/1) to give tert-butyl N-(8-bromochroman-6-yl)carbamate (780 mg, crude) as a yellow solid.

### Step 8. 8-bromochroman-6-amine

To a solution of tert-butyl N-(8-bromochroman-6-yl)carbamate (370 mg, 1.13 mmol) in DCM (5 mL) was added TFA (1.48 g, 12.98 mmol, 1 mL), then the mixture was stirred at 20° C. for 2 h. LCMS showed desired mass was detected. The reaction was concentrated under reduced pressure to give 8-bromochroman-6-amine (260 mg, crude) as yellow oil.

### Step 9. N2-(8-bromochroman-6-yl)-N4,6-dimethyl-pyrimidine-2,4-diamine

To a solution of 8-bromochroman-6-amine (65 mg, 284.98 μmol) and 2-chloro-N, 6-dimethyl-pyrimidin-4-amine (44.91 mg, 284.98 μmol) in i-PrOH (3 mL) was added HCl (12 M, 2.37 μL), then the mixture was stirred at 130° C. in the microwave for 1 hr. LCMS showed desired mass was detected. The four batch reactions were work up together. The combined reaction mixture was concentrated to remover i-PrOH, the residue was added to water/EtOAc (50 mL, v:v=1:1). The mixture was acidified by sat. NaHCO₃ till pH=8. The mixture was extracted with EtOAc (20 mL×6). The organic layer was dried over Na₂SO₄, concentrated to give N2-(8-bromochroman-6-yl)-N4,6-dimethyl-pyrimidine-2,4-diamine (370 mg, crude) as yellow solid.

### Step 10. tert-butyl5-[6-[[4-methyl-6-(methylamino)pyrimidin-2-yl]amino]chroman-8-yl]-2,3,4,7-tetrahydroazepine-1-carboxylate

To a solution of N2-(8-bromochroman-6-yl)-N4,6-dimethyl-pyrimidine-2,4-diamine (50 mg, 143.17 mol) and tert-butyl 5-(4,4,5,5-tetramethyl-1,3,2-dioxaborolan-2-yl)-2,3,4,7-tetrahydroazepine-1-carboxylate (50.91 mg, 157.49 μmol) in dioxane (2 mL) and H₂O (0.2 mL) was added tripotassium carbonate (39.57 mg, 286.35 mol, 17.28 μL) and cyclopentyl(diphenyl)phosphane dichloropalladium iron (10.48 mg, 14.32 μmol), then the mixture was stirred at 100° C. for 12 h under N2. LCMS showed desired mass was detected. The reaction was filtered and concentrated under reduced pressure to give tert-butyl 5-[6-[[4-methyl-6-(methylamino)pyrimidin-2-yl]amino]chroman-8-yl]-2,3,4,7-tetrahydroazepine-1-carboxylate (60 mg, crude) as black solid.

### Step 11. N4,6-dimethyl-N2-[8-(2, 3, 4, 7-tetrahydro-1H-azepin-5-yl)chroman-6-yl]pyrimidine-2,4-diamine

To a solution of tert-butyl 5-[6-[[4-methyl-6-(methylamino) pyrimidin-2-yl]amino]chroman-8-yl]-2,3,4,7-tetrahydroazepine-1-carboxylate (60 mg, 128.87 μmol) in DCM (1 mL) was added TFA (1.48 g, 12.98 mmol, 1 mL), then the mixture was stirred at 20° C. for 1 h. LCMS showed desired mass was detected. The reaction was filtered and concentrated under reduced pressure to give a residue. The crude product was purified by prep-HPLC(TFA condition:Phenomenex Luna 80*30 mm*3 um; mobile phase:water(0.1% TFA)-ACN]; B %: 5%-35%,8 min) to give N4,6-dimethyl-N2-[8-(2,3,4,7-tetrahydro-1H-azepin-5-yl)chroman-6-yl]pyrimidine-2,4-diamine (27.2 mg, 56.85 μmol, 44.11% yield, TFA) as black solid.

¹H NMR (400 MHz, METHANOL-d₄) δ ppm 7.29 (br d, J=1.1 Hz, 1H), 7.10 (d, J=1.8 Hz, 1H), 5.95 (s, 1H), 5.84 (t, J=6.4 Hz, 1H), 4.24-4.19 (m, 2H), 3.86 (d, J=6.6 Hz, 2H), 3.49-3.45 (m, 2H), 2.97 (s, 3H), 2.83 (t, J=6.5 Hz, 2H), 2.75-2.70 (m, 2H), 2.28 (s, 3H), 2.08-2.00 (m, 4H). MS (ESI): m/z=366.1 [M+H]⁺.

### Step 12. N⁴,6-dimethyl-N²-[7-(3-pyrrolidin-1-ylpropoxy)-2,3-dihydrobenzofuran-5-yl]pyrimidine-2,4-diamine

To a solution of N4,6-dimethyl-N2-[8-(2,3,4,7-tetrahydro-1H-azepin-5-yl)chroman-6-yl]pyrimidine-2,4-diamine (40 mg, 109.45 μmol) in MeOH (3 mL) was added formaldehyde (16.43 mg, 547.24 μmol, 15.17 μL) and sodium; cyanoboranuide (13.76 mg, 218.90 μmol), then the mixture was stirred at 20° C. for 12 h. LCMS showed desired mass was detected. The reaction was filtered and concentrated under reduced pressure to give a residue. The crude product was purified by prep-HPLC(TFA condition:Phenomenex Luna 80*30 mm*3 um; mobile phase:water (TFA)-ACN]; B %: 5%-35%,8 min) to give N4,6-dimethyl-N2-[8-(1-methyl-2,3,4,7-tetrahydroazepin-5-yl)chroman-6-yl]pyrimidine-2,4-diamine (32.5 mg, 65.99 μmol, 60.29% yield, TFA) as pale yellow solid.

¹H NMR (400 MHz, METHANOL-d₄) δ ppm 7.31 (br s, 1H), 7.12 (br d, J=1.4 Hz, 1H), 5.95 (s, 1H), 5.79 (t, J=6.7 Hz, 1H), 4.22 (br s, 2H), 4.03 (br d, J=5.4 Hz, 1H), 3.94 (br d, J=7.6 Hz, 1H), 3.68 (br d, J=12.0 Hz, 1H), 3.44 (br dd, J=6.3, 12.5 Hz, 1H), 2.97 (s, 3H), 2.94-2.91 (m, 3H), 2.83 (br t, J=6.4 Hz, 2H), 2.72 (br dd, J=4.8, 9.1 Hz, 2H), 2.28 (s, 3H), 2.13-1.99 (m, 4H). MS (ESI): m/z=380.2 [M+H]⁺.

### EXAMPLE 18. Synthesis of Compound 122

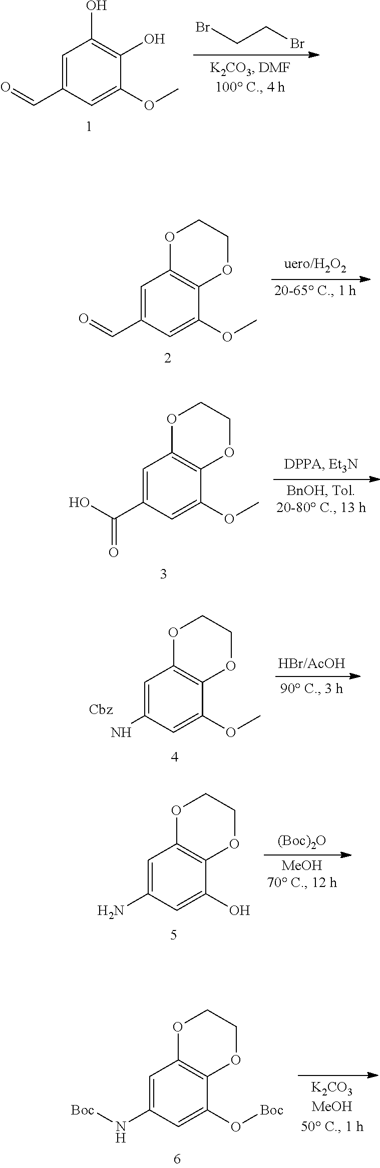

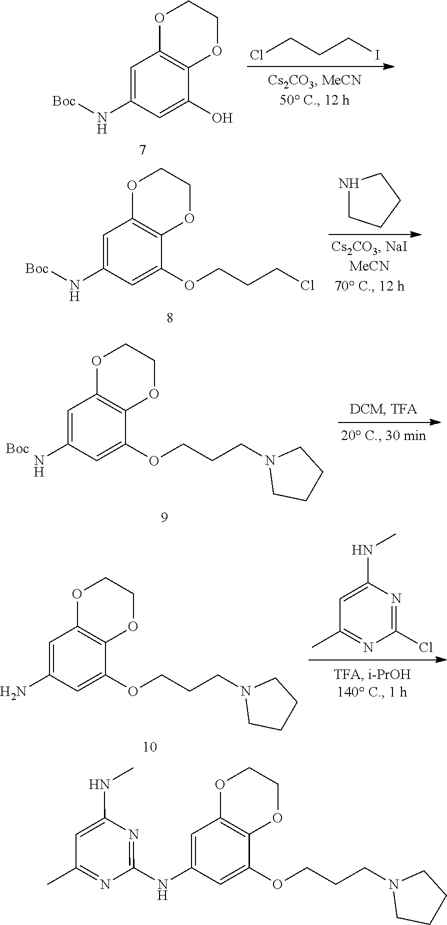

### Step 1. 5-methoxy-2,3-dihydro-1,4-benzodioxine-7-carbaldehyde

To a solution of 3,4-dihydroxy-5-methoxy-benzaldehyde (4 g, 23.79 mmol) in DMF (60 mL) was added 1,2-dibromoethane (4.92 g, 26.17 mmol, 2.25 mL) and K₂CO₃ (6.58 g, 47.58 mmol). It was stirred at 100° C. for 4 h. TLC (petroleum ether:ethyl acetate=1:1, Rf=0.5) indicated starting material was consumed completely, and one major new spot was detected. LCMS showed starting material was consumed completely and mass of the desired compound. The reaction was added into water (200 mL), then extracted with ethyl acetate (3×200 mL). The combined organic layers were washed with brine (3×200 mL) and dried over anhydrous sodium sulfate, filtered and concentrated under reduced pressure to give 5-methoxy-2,3-dihydro-1,4-benzodioxine-7-carbaldehyde (3.9 g, crude) as a light-yellow solid.

### Step 2. 5-methoxy-2,3-dihydro-1,4-benzodioxine-7-carboxylic acid

Aqueous NaOH (6 M, 5.4 mL) was added dropwise to a stirred solution of the hydrogen peroxide urea (25 g, 265.76 mmol) and aldehyde 5-methoxy-2,3-dihydro-1,4-benzodioxine-7-carbaldehyde (3 g, 15.45 mmol) in MeOH (60 mL) at 20° C. The resulting mixture was stirred at 65° C. for 1 h. LCMS showed starting material was consumed completely and mass of the desired compound. The reaction was added saturated sodium thiosulfate aqueous solution (20 mL) at 0° C., then the mixture was stirred at 20° C. for 15 min, then concentrated under reduced pressure to dry MeOH, then was added 1 N HCl adjust to pH=5-6, filtered and the filter cake was concentrated under reduced pressure to give 5-methoxy-2,3-dihydro-1,4-benzodioxine-7-carboxylic acid (2.7 g, 12.85 mmol, 83.15% yield) as a white solid.

### Step 3. benzyl N-(5-methoxy-2,3-dihydro-1,4-benzodioxin-7-yl)carbamate

To a solution of 5-methoxy-2,3-dihydro-1,4-benzodioxine-7-carboxylic acid (2.7 g, 12.85 mmol) in toluene (30 mL) was added DPPA (3.75 g, 15.42 mmol), TEA (3.90 g, 38.54 mmol, 5.37 mL). It was stirred at 20° C. for 1 h. Then it was added phenyl methanol (4.17 g, 38.54 mmol, 3.99 mL). It was stirred at 80° C. for 12 h. TLC (petroleum ether:ethyl acetate=3:1, Rf=0.2) indicated starting material was consumed completely, and two major new spots were detected. LCMS showed starting material was consumed completely and mass of the desired compound. The reaction was added water (50 mL), then extracted with ethyl acetate (3*50 mL) and dried over anhydrous sodium sulfate, filtered and concentrated under reduced pressure to give a residue. The residue was purified by flash chromatography (silica gel, petroleum ether/ethyl acetate=1/0 to 1/1) to give crude product. The crude product was triturated with petroleum ether (50 mL) at 20° C. for 20 min. The mixture was filtered, then the filter cake was concentrated under reduced pressure to give benzyl N-(5-methoxy-2,3-dihydro-1,4-benzodioxin-7-yl)carbamate (2 g, 6.34 mmol, 57.14% yield) as an off-white solid.

### Step 4. 7-amino-2,3-dihydro-1,4-benzodioxin-5-ol

A solution of benzyl N-(5-methoxy-2,3-dihydro-1,4-benzodioxin-7-yl)carbamate (500 mg, 1.59 mmol) in HBr/AcOH (5 mL) was stirred at 90° C. for 3 h. LCMS showed starting material was consumed completely and mass of the desired compound. The reaction was concentrated under reduced pressure, then added saturated sodium bicarbonate solution adjust to pH=7-8, then concentrated under reduced pressure to give a residue. The residue was added dichloromethane (5 mL), filtered and concentrated under reduced pressure to give 7-amino-2,3-dihydro-1,4-benzodioxin-5-ol (270 mg, crude) as a light-yellow solid

### Step 5. [7-(tert-butoxycarbonylamino)-2,3-dihydro-1,4-benzodioxin-5-yl]tert-butyl carbonate

To a solution of 7-amino-2,3-dihydro-1,4-benzodioxin-5-ol (250 mg, 1.50 mmol) in MeOH (2 mL) was added tert-butoxycarbonyl tert-butyl carbonate (979.20 mg, 4.49 mmol, 1.03 mL). It was stirred at 70° C. for 12 h. LCMS showed starting material was consumed completely and mass of the desired compound. The reaction was filtered and concentrated under reduced pressure to give [7-(tert-butoxycarbonylamino)-2,3-dihydro-1,4-benzodioxin-5-yl]tert-butyl carbonate (550 mg, crude) as a brown solid.

### Step 6. tert-butyl N-(5-hydroxy-2,3-dihydro-1,4-benzodioxin-7-yl)carbamate

To a solution of [7-(tert-butoxycarbonylamino)-2,3-dihydro-1,4-benzodioxin-5-yl]tert-butyl carbonate (550 mg, 1.50 mmol) in MeOH (5 mL) was added K₂CO₃ (413.80 mg, 2.99 mmol). It was stirred at 50° C. for 1 h. TLC (petroleum ether:ethyl acetate=3:1, Rf=0.2) indicated starting material was consumed completely, and one major new spot was detected. LCMS showed starting material was consumed completely and mass of the desired compound. The reaction was concentrated under reduced pressure, then added water (10 mL), then extracted with ethyl acetate (3×10 mL) and dried over anhydrous sodium sulfate, filtered and concentrated under reduced pressure to give a residue. The residue was purified by prep-TLC (SiO₂, petroleum ether:ethyl acetate=3:1) to give tert-butyl N-(5-hydroxy-2,3-dihydro-1,4-benzodioxin-7-yl)carbamate (220 mg, 823.12 μmol, 54.98% yield) as a brown oil.

### Step 7. tert-butyl N-[5-(3-chloropropoxy)-2,3-dihydro-1,4-benzodioxin-7-yl]carbamate

To a solution of tert-butyl N-(5-hydroxy-2,3-dihydro-1,4-benzodioxin-7-yl)carbamate (110 mg, 411.56 μmol) and 1-chloro-3-iodopropane (84.14 mg, 411.56 μmol, 44.19 μL) in MeCN (2 mL) was added C_{S2}CO₃ (268.19 mg, 823.12 mol). It was stirred at 50° C. for 12 h. LCMS showed starting material was consumed completely and mass of the desired compound. The reaction was filtered and concentrated under reduced pressure to give tert-butyl N-[5-(3-chloropropoxy)-2,3-dihydro-1,4-benzodioxin-7-yl]carbamate (140 mg, crude) as a brown oil.

### Step 8. tert-butyl N-[5-(3-pyrrolidin-1-ylpropoxy)-2,3-dihydro-1,4-benzodioxin-7-yl]carbamate

To a solution of tert-butyl N-[5-(3-chloropropoxy)-2,3-dihydro-1,4-benzodioxin-7-yl]carbamate (140 mg, 407.21 mol), NaI (61.04 mg, 407.21 mol, 16.65 μL) and C_{S2}CO₃ (265.35 mg, 814.42 μmol) in MeCN (3 mL) was added pyrrolidine (28.96 mg, 407.21 μmol, 33.83 μL). It was stirred at 70° C. for 12 h. LCMS showed starting material was consumed completely and mass of the desired compound. The reaction was filtered and concentrated under reduced pressure to give tert-butyl N-[5-(3-pyrrolidin-1-ylpropoxy)-2,3-dihydro-1,4-benzodioxin-7-yl]carbamate (155 mg, crude) as a brown oil.

### Step 9. 5-(3-pyrrolidin-1-ylpropoxy)-2,3-dihydro-1,4-benzodioxin-7-amine

To a solution of tert-butyl N-[5-(3-pyrrolidin-1-ylpropoxy)-2,3-dihydro-1,4-benzodioxin-7-yl]carbamate (50 mg, 132.11 μmol) in DCM (0.5 mL) was added TFA (0.25 mL). It was stirred at 20° C. for 30 min. LCMS showed starting material was consumed completely and mass of the desired compound. The reaction was filtered and concentrated under reduced pressure to give 5-(3-pyrrolidin-1-ylpropoxy)-2,3-dihydro-1,4-benzodioxin-7-amine (40 mg, crude) as a brown oil.

### Step 10. N4,6-dimethyl-N2-[5-(3-pyrrolidin-1-ylpropoxy)-2,3-dihydro-1,4-benzodioxin-7-v/l pyrimidine-2,4-diamine

To a solution of 5-(3-pyrrolidin-1-ylpropoxy)-2,3-dihydro-1,4-benzodioxin-7-amine (40 mg, 143.71 mol) and 2-chloro-N,6-dimethyl-pyrimidin-4-amine (22.65 mg, 143.71 μmol) in i-PrOH (1 mL) was added TFA (1.64 mg, 14.37 μmol, 1.11 μL), the reaction was stirred at 140° C. for 1 h. LCMS showed starting material was consumed completely and mass of the desired compound. The reaction was filtered and concentrated under reduced pressure to give a residue. The residue was purified by prep-HPLC (TFA condition, column: Phenomenex luna C¹⁸ 100*40 mm*5 um; mobile phase: [water(0.1% TFA)-ACN]; B %: 1%-30%, 8 min) to give N4,6-dimethyl-N2-[5-(3-pyrrolidin-1-ylpropoxy)-2,3-dihydro-1,4-benzodioxin-7-yl]pyrimidine-2,4-diamine (2.5 mg, 6.26 μmol, 4.35% yield) (purity: 100%) as a white solid.

¹H NMR (400 MHz, METHANOL-d₄) δ ppm 2.12 (br s, 4H) 2.24 (br s, 5H) 2.96 (s, 3H) 3.44 (br s, 6H) 4.18 (br s, 2H) 4.26 (s, 4H) 5.89 (s, 1H) 6.91 (m, 1H) 7.04 (s, 1H).

### EXAMPLE 19. Synthesis of Compound 124

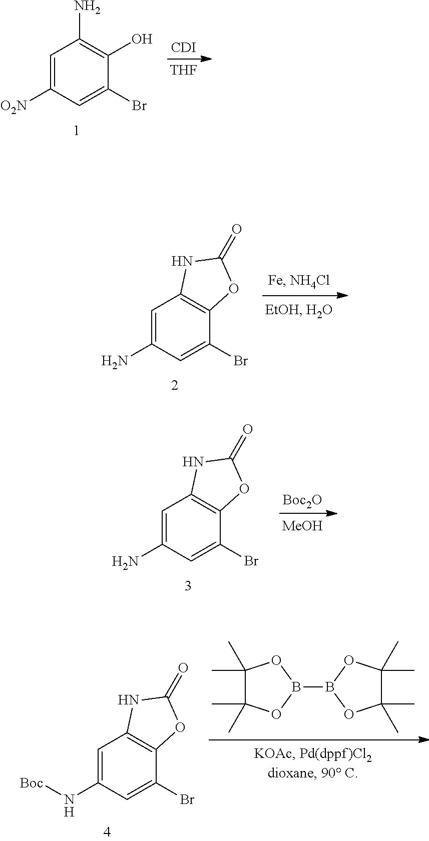

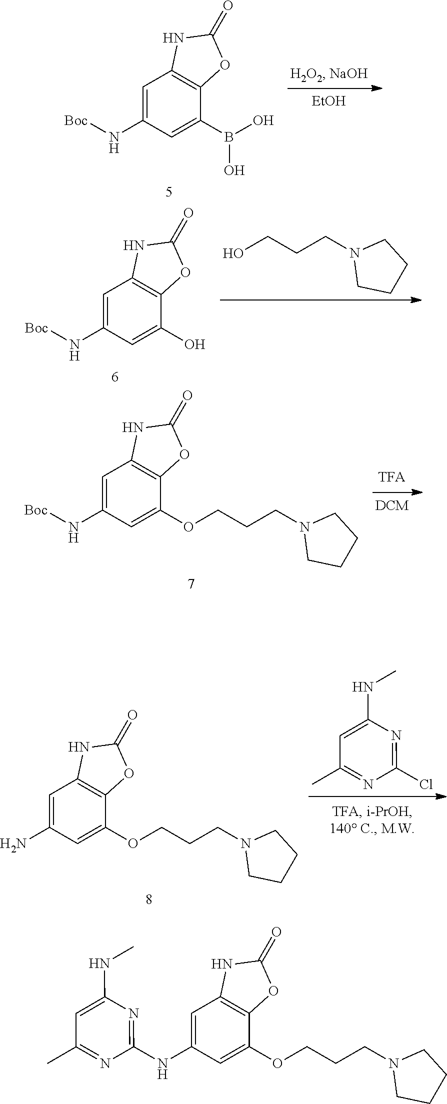

### Step 1. 7-bromo-5-nitro-3H-1,3-benzoxazol-2-one

To a solution of 2-amino-6-bromo-4-nitro-phenol (1 g, 4.29 mmol) in THF (10 mL) was added carbonyldiimidazole (835.03 mg, 5.15 mmol), then the mixture was stirred at 70° C. for 1 hr. TLC (petroleum ether:ethyl acetate=2:1, Rf=0.4) indicated starting material was consumed completely, and one major new spot was detected. The reaction mixture was quenched by water (10 mL), extracted with EtOAc (10 mL*4). The organic layer was dried over Na₂SO₄, concentrated to give 7-bromo-5-nitro-3H-1,3-benzoxazol-2-one (1.2 g, crude) as black solid.

### Step 2. 5-amino-7-bromo-3H-1,3-benzoxazol-2-one

To a solution of 7-bromo-5-nitro-3H-1,3-benzoxazol-2-one (1.2 g, 4.63 mmol) in EtOH (32 mL) and H₂O (8 mL) was added Fe (2.59 g, 46.33 mmol) and NH₄Cl (2.48 g, 46.33 mmol), then the mixture was stirred at 80° C. for 1 hr. LCMS showed starting material was consumed completely and desired MS was detected. The reaction was cooled to 50° C., and then the mixture was filtered, and the filtered caked was washed with MeOH (10 mL*3), and the filtrated was concentrated in vacuum at 50° C. to give 5-amino-7-bromo-3H-1,3-benzoxazol-2-one (1.1 g, crude) as black solid.

### Step 3. tert-butyl N-(7-bromo-2-oxo-3H-1,3-benzoxazol-5-yl)carbamate

To a solution of 5-amino-7-bromo-3H-1,3-benzoxazol-2-one (1.1 g, 4.80 mmol) in MeOH (26.20 mL) was added tert-butoxycarbonyl tert-butyl carbonate (3.14 g, 14.41 mmol, 3.31 mL), then the mixture was stirred at 60° C. for 12 hrs. LCMS showed starting material was consumed completely and desired MS was detected. The reaction was filtered and concentrated under reduced pressure to give a residue. The residue was purified by column chromatography (SiO₂, Petroleum ether/Ethyl acetate=1/0 to 0/1) to give tert-butyl N-(7-bromo-2-oxo-3H-1,3-benzoxazol-5-yl)carbamate (390 mg, crude) as yellow solid.

### Step 4. [5-(tert-butoxycarbonylamino)-2-oxo-3H-1,3-benzoxazol-7-yl]boronic acid

Tert-butyl N-(7-bromo-2-oxo-3H-1,3-benzoxazol-5-yl)carbamate (250 mg, 759.54 μmol) was dissolved indioxane (4 mL), and then to the mixture was added 4,4,5,5-tetramethyl-2-(4,4,5,5-tetramethyl-1,3,2-dioxaborolan-2-yl)-1,3,2-dioxaborolane (231.45 mg, 911.45 μmol) KOAc (428.53 mg, 1.52 mmol), cyclopentyl(diphenyl)phosphane dichloromethane; dichloropalladium;iron (62.03 mg, 75.95 μmol), and then the mixture was stirred at 100° C. for 12 h. LCMS showed starting material was consumed completely and desired MS was detected. The mixture was concentrated in vacuum. [5-(tert-butoxycarbonylamino)-2-oxo-3H-1,3-benzoxazol-7-yl]boronic acid (220 mg, crude) was obtained as a black solid.

### Step 5. tert-butyl N-(7-hydroxy-2-oxo-3H-1,3-benzoxazol-5-yl)carbamate

[5-(Tert-butoxycarbonylamino)-2-oxo-3H-1,3-benzoxazol-7-yl]boronic acid (220 mg, 748.13 μmol) was dissolved in EtOH (5 mL), and then to the mixture was added H₂O₂ (169.65 mg, 1.50 mmol, 30% purity) at 0° C., and then to the mixture was stirred at 25° C. for 1 h. LCMS showed starting material was consumed completely and desired MS was detected. The residue was purified by flash chromatography (silica gel, petroleum ether/ethyl acetate=50/1 to 1/1 get the spot, 0/1). tert-butyl N-(7-hydroxy-2-oxo-3H-1,3-benzoxazol-5-yl)carbamate (199 mg, 747.42 μmol, 99.91% yield) was obtained as a yellow solid.

### Step 6. tert-butyl N-[2-oxo-7-(3-pyrrolidin-1-ylpropoxy)-3H-1,3-benzoxazol-5-yl]carbamate

Tert-butyl N-(7-hydroxy-2-oxo-3H-1,3-benzoxazol-5-yl)carbamate (50 mg, 187.79 μmol) 3-pyrrolidin-1-ylpropan-1-ol (16.98 mg, 131.46 μmol), triphenylphosphine (54.18 mg, 206.57 μmol) was dissolved in THF (2 mL), and then to the mixture was added isopropyl (NE)-N-isopropoxycarbonyliminocarbamate (41.77 mg, 206.57 μmol, 40.67 μL) in THF (0.5 mL), and then the mixture was stirred at 25° C. for 12 h. LCMS showed starting material was consumed completely and desired MS was detected. It was purified by preparative-HPLC (TFA condition, column: 3 Phenomenex Luna C18 75*30 mm*3 um; mobile phase: [water(0.1% TFA)-ACN]; B %: 1%-35%,8 min. tert-butyl N-[2-oxo-7-(3-pyrrolidin-1-ylpropoxy)-3H-1,3-benzoxazol-5-yl]carbamate (10 mg, 20.39 μmol, 10.86% yield, TFA) was obtained as a black solid.

### Step 7. 5-amino-7-(3-pyrrolidin-1-ylpropoxy)-3H-1,3-benzoxazol-2-one

Tert-butyl N-[2-oxo-7-(3-pyrrolidin-1-ylpropoxy)-3H-1,3-benzoxazol-5-yl]carbamate (10 mg, 20.39 mol, TFA) was dissolved in DCM (2.09 mL), and then to the mixture was added TFA (455.58 mg, 4.00 mmol, 307.83 μL), and then the mixture was stirred at 25° C. for 1 h. LCMS showed starting material was consumed completely and desired MS was detected. The mixture was concentrated in vacuo. 5-amino-7-(3-pyrrolidin-1-ylpropoxy)-3H-1,3-benzoxazol-2-one (7 mg, crude, TFA) was obtained as a yellow oil.

### Step 8. 5-[[4-methyl-6-(methylamino)pyrimidin-2-yl]amino]-7-(3-pyrrolidin-1-ylpropoxy)-3H-1,3-benzoxazol-2-one

5-Amino-7-(3-pyrrolidin-1-ylpropoxy)-3H-1,3-benzoxazol-2-one (7 mg, 17.93 μmol, TFA), 2-chloro-N,6-dimethyl-pyrimidin-4-amine (2.83 mg, 17.93 μmol) was dissolved in i-PrOH (2 mL), and then the mixture was stirred at 120° C. for 30 min under microwave. LCMS showed starting material was consumed completely and desired MS was detected. It was purified by preparative-HPLC (TFA condition, column: 3_Phenomenex Luna C18 75*30 mm*3 um; mobile phase: [water(0.1% TFA)-ACN]; B %: 3%-35%,8 min. 5-[[4-methyl-6-(methylamino)pyrimidin-2-yl]amino]-7-(3-pyrrolidin-1-ylpropoxy)-3H-1,3-benzoxazol-2-one (1.6 mg, 3.13 μmol, 17.44% yield, TFA) was obtained as a gray solid.

¹H NMR (400 MHz, METHANOL-d₄) δ ppm 7.29 (s, 1H), 7.02 (d, J=1.7 Hz, 1H), 6.05 (s, 1H), 4.36 (t, J=5.6 Hz, 2H), 3.85-3.72 (m, 2H), 3.52-3.47 (m, 2H), 3.22-3.13 (m, 2H), 3.05 (s, 3H), 2.35-2.29 (m, 5H), 2.27-2.18 (m, 2H), 2.09 (br d, J=7.0 Hz, 2H). MS (ESI): m/z=399.1 [M+H]⁺

### EXAMPLE 20. Synthesis of Compounds 125 and 126

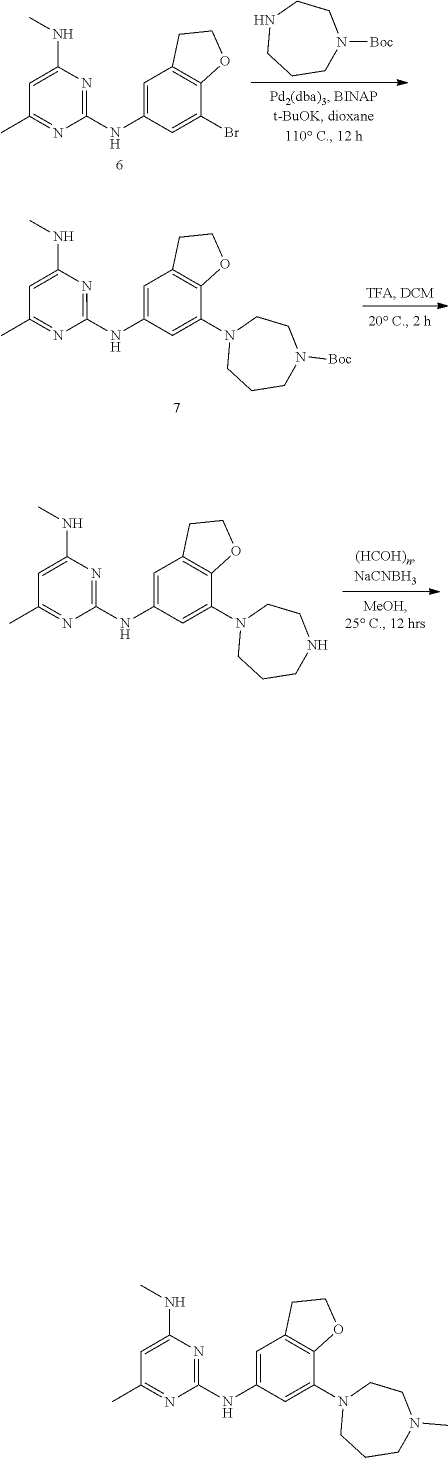

### Step 1. tert-butyl4-[5-[[4-methyl-6-(methylamino)pyrimidin-2-yl]amino]-2,3-dihydrobenzofuran-7-yl]-1,4-diazepane-1-carboxylate

To a solution of N2-(7-bromo-2,3-dihydrobenzofuran-5-yl)-N4,6-dimethyl-pyrimidine-2,4-diamine (50 mg, 149.17 μmol) in dioxane (1 mL) was added tert-butyl 1,4-diazepane-1-carboxylate (74.69 mg, 372.91 mol), benzyl-[1-[2-[benzyl(phenyl) phosphanyl]-1-naphthyl]-2-naphthyl]-phenyl-phosphane (38.83 mg, 59.67 mol) and CS₂CO₃ (145.80 mg, 447.50 μmol), then it was added Pd₂(dba)₃ (27.32 mg, 29.83 mol) under N₂ atmosphere. The reaction was stirred at 100° C. for 24 hours under N₂ atmosphere. LCMS showed starting material was remained and mass of the desired compound. Then it was concentrated under reduced pressure to give tert-butyl4-[5-[[4-methyl-6-(methylamino)pyrimidin-2-yl]amino]-2,3-dihydrobenzofuran-7-yl]-1,4-diazepane-1-carboxylate (70 mg, crude) as a black oil.

### Step 2. N2-[7-(1,4-diazepan-1-yl]-2,3-dihydrobenzofuran-5-yl]-N4,6-dimethyl-pyrimidine-2,4-diamine

To a solution of tert-butyl 4-[5-[[4-methyl-6-(methylamino)pyrimidin-2-yl]amino]-2,3-dihydrobenzofuran-7-yl]-1,4-diazepane-1-carboxylate (70 mg, 153.99 μmol) in TFA (0.5 mL) and DCM (1 mL) was stirred at 20° C. for 1 hour. LCMS showed starting material was consumed completely and mass of the desired compound. One third of the reaction mixture was purified by prep-HPLC (TFA conditions; column: Phenomenex luna C18 100*40 mm*5 um; mobile phase: [water(0.1% TFA)-ACN]; B %: 1%-27%, 8 min) to give N2-[7-(1,4-diazepan-1-yl)-2,3-dihydrobenzofuran-5-yl]-N4,6-dimethyl-pyrimidine-2,4-diamine (2.0 mg, 5.64 μmol, 3.66% yield) (100% purity, TFA salt) as a white solid. The other of the reaction mixture was concentrated to give N2-[7-(1,4-diazepan-1-yl)-2,3-dihydrobenzofuran-5-yl]-N4,6-dimethyl-pyrimidine-2,4-diamine (8 mg, crude) as a yellow oil.

¹H NMR (400 MHz, METHANOL-d₄) δ ppm 2.19-2.26 (m, 2H) 2.28 (s, 3H) 2.99 (s, 3 H)3.24 (t, J=8.69 Hz, 2H)3.37-3.41 (m, 2H)3.42-3.47 (m, 4H)3.61-3.65 (m, 2H)4.61 (t, J=8.76 Hz, 2H) 5.96 (s, 1H) 6.86 (s, 1H) 7.00 (s, 1H).

### Step 3. N2-[7-(1,4-diazepan-1-yl)-2,3-dihydrobenzofuran-5-yl]-N4,6-dimethyl pyrimidine-2,4-diamine

To a solution of N2-[7-(1,4-diazepan-1-yl)-2,3-dihydrobenzofuran-5-yl]-N4,6-dimethyl-pyrimidine-2,4-diamine (8 mg, 22.57 μmol) was dissolved in MeOH (2 mL), and it was added AcOH to pH=5. It was added (HCHO)ₙ(2.03 mg, 67.71 μmol) and NaBH₃CN (5.67 mg, 90.28 mol) and was stirred at 20° C. for 12 hours. LCMS showed starting material was consumed completely and mass of the desired compound. Then it was concentrated under reduced pressure to give a residue. It was purified by prep-HPLC (TFA condition, column:Phenomenex luna C18 100*40 mm*5 um: [water(0.1% TFA)-ACN]; B %: 1%-30%, 8 min) to give N4,6-dimethyl-N2-[7-(4-methyl-1,4-diazepan-1-yl)-2,3-dihydrobenzofuran-5-yl]pyrimidine-2,4-diamine (5.4 mg, 14.66 μmol, 64.93% yield) (100% purity, TFA salt) as a white solid.

¹H NMR (400 MHz, METHANOL-d₄) δ ppm 2.26 (s, 5H) 2.97 (d, J=2.00 Hz, 6H) 3.18-3.26 (m, 2H) 3.35-3.58 (m, 6H) 3.58-3.73 (m, 2H) 4.60 (br t, J=8.63 Hz, 2H) 5.94 (s, 1H) 6.83 (br s, 1H) 6.96 (s, 1H).

### EXAMPLE 21. Synthesis of Compound 127

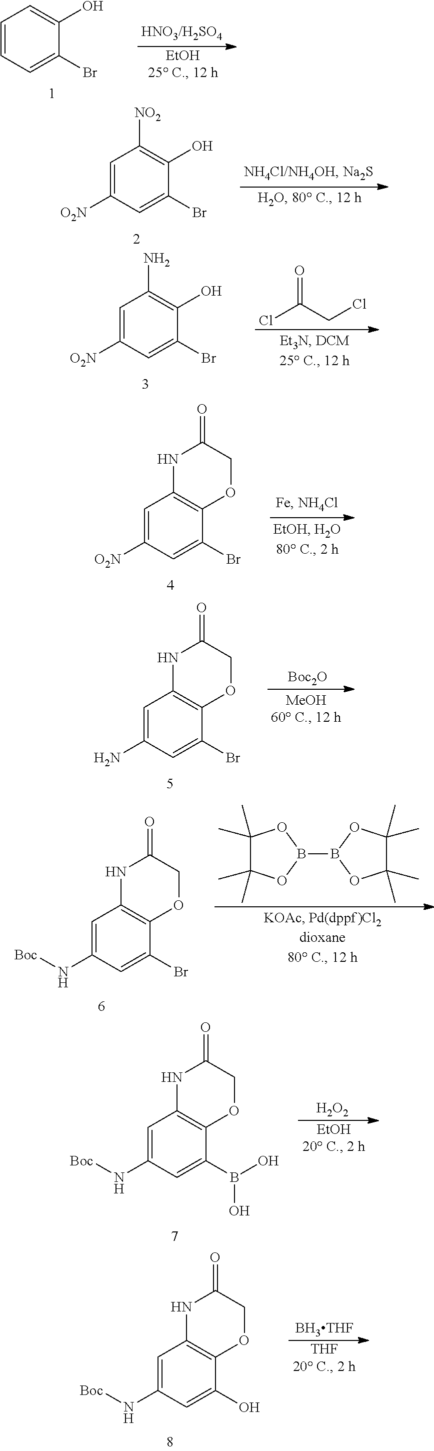

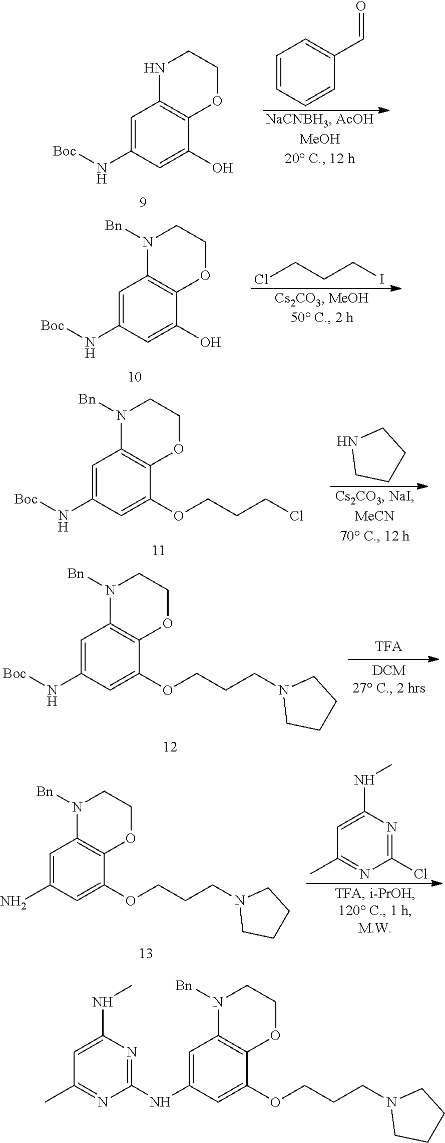

### Step 1. 2-bromo-4,6-dinitro-phenol

2-Bromophenol (10 g, 57.80 mmol, 6.70 mL) was dissolved in EtOH (50 mL) and H₂SO₄ (100 mL), and then to the mixture was added HNO₃ (30.00 g, 476.09 mmol, 20 mL) very slowly, and then the mixture was stirred at 25° C. for 12 hrs. TLC(petroleum ether:ethyl acetate=0:1, Rf=0.5) indicated starting material was consumed completely, and one major new spot was detected. The mixture was added to ice (500 mL) at 5° C., and then the suspension was filtered, and washed with H₂O (50 mL*2), and the filtered cake was the desired compound, and the filtrate was quenched by sat. 2M NaOH to pH=7 at 25° C. slowly. 2-bromo-4,6-dinitro-phenol (12 g, 45.63 mmol, 78.94% yield) was obtained as a yellow solid.

### Step 2. 2-amino-6-bromo-4-nitro-phenol

NH₄Cl (8.00 g, 149.56 mmol) and NH₄OH (2.13 g, 15.21 mmol, 2 mL, 25% purity) were added to a solution of 2-bromo-4,6-dinitro-phenol (4 g, 15.21 mmol) in H₂O (100 mL). The mixture was heated to 80° C. Sodiosulfanylsodiumnonahydrate (4.40 g, 18.32 mmol) was added, After addition, reaction was heated for 12 hrs at 80° C. LCMS showed starting material was consumed completely and desired MS was detected. TLC (petroleum ether:ethyl acetate=3:1, Rf=0.1) indicated starting material was consumed completely, and one major new spot was detected. The mixture was diluted with H₂O (200 mL), and then to the mixture was added AcOH to pH=2 at 10° C., and then extracted with EtOAc(100 mL*3), and then the organic phase was concentrated in vacuum. 2-amino-6-bromo-4-nitro-phenol (3.5 g, crude) was obtained as a black solid.

### Step 3. 8-bromo-6-nitro-4H-1,4-benzoxazin-3-one

2-Amino-6-bromo-4-nitro-phenol (1 g, 4.29 mmol) was dissolved in DCM (20 mL) and then to the mixture was added TEA (1.30 g, 12.87 mmol, 1.79 mL), 2-chloroacetyl chloride (581.63 mg, 5.15 mmol, 410.47 μL) and then the mixture was stirred at 25° C. for 12 hrs LCMS showed starting material was consumed completely and a main peak was detected. TLC(petroleum ether:ethyl acetate=5:1, Rf=0.1) indicated starting material was consumed completely, and one major new spot was detected. The reaction was diluted by H₂O (100 mL), and then extracted with DCM (20 mL*2), the organic phase was concentrated in vacuum. 8-bromo-6-nitro-4H-1,4-benzoxazin-3-one (1.1 g, crude) was obtained as a yellow oil.

### Step 4. 6-amino-8-bromo-4H-1,4-benzoxazin-3-one

To a solution of 8-bromo-6-nitro-4H-1,4-benzoxazin-3-one (1.8 g, 6.59 mmol) in EtOH (20 mL) and H₂O (5 mL) was added NH₄Cl (3.53 g, 65.92 mmol) and Fe (3.68 g, 65.92 mmol), then the mixture was stirred at 80° C. for 2 hrs. LCMS showed the reaction was complete and the desired ms was detected. The reaction was cooled to 50° C., and the filtered, the filter cake was washed with MeOH (100 mL*3). The filtrate was concentrated in vacuum at 50° C. 6-amino-8-bromo-4H-1,4-benzoxazin-3-one (4 g, crude) was obtained as a black solid.

### Step 5. tert-butyl N-(8-bromo-3-oxo-4H-1,4-benzoxazin-6-yl)carbamate

To a solution of 6-amino-8-bromo-4H-1,4-benzoxazin-3-one (4 g, 16.46 mmol) in MeOH (40 mL) was added tert-butoxycarbonyl tert-butyl carbonate (10.78 g, 49.37 mmol, 11.33 mL), then the mixture was stirred at 60° C. for 12 hrs. LCMS showed the reaction was complete and the desired ms was detected. The reaction was concentrated under reduced pressure to give a residue. The residue was purified by column chromatography (SiO₂, Petroleum ether/Ethyl acetate=1/0 to 0/1) to give tert-butyl N-(8-bromo-3-oxo-4H-1,4-benzoxazin-6-yl)carbamate (900 mg, crude) as yellow solid.

### Step 6. [6-(tert-butoxycarbonylamino)-3-oxo-4H-1,4-benzoxazin-8-yl]boronic acid

Tert-butyl N-(8-bromo-3-oxo-4H-1,4-benzoxazin-6-yl)carbamate (900 mg, 2.62 mmol) was dissolved in dioxane (9 mL) and the to the mixture was added 4,4,5,5-tetramethyl-2-(4,4,5,5-tetramethyl-1,3,2-dioxaborolan-2-yl)-1,3,2-dioxaborolane (799.17 mg, 3.15 mmol), KOAc (1.48 g, 5.25 mmol) and cyclopentyl(diphenyl)phosphane dichloropalladium iron (191.90 mg, 262.26 mol), and then the mixture was stirred at 80° C. for 12 hrs under N2. LCMS showed the reaction was complete and the desired ms was detected. The reaction was concentrated under reduced pressure to give [6-(tert-butoxycarbonylamino)-3-oxo-4H-1,4-benzoxazin-8-yl]boronic acid (1 g, crude) as brown solid.

### Step 7. tert-butyl N-(8-hydroxy-3-oxo-4H-1,4-benzoxazin-6-yl)carbamate

[6-(Tert-butoxycarbonylamino)-3-oxo-4H-1,4-benzoxazin-8-yl]boronic acid (1 g, 3.25 mmol) was dissolved in EtOH (30 mL), and to the mixture was added H₂O₂ (736.02 mg, 6.49 mmol, 663.08 μL, 30% purity) at 0° C., and then the mixture was stirred at 20° C. for 2 hrs. LCMS showed the reaction was complete and the desired ms was detected. The reaction was concentrated under reduced pressure to give a residue. The reaction mixture was quenched by sat. NaHSO₃ (40 mL), extracted with EtOAc (30 mL*6). The organic layer was dried over Na₂SO₄, concentrated to give the crude product. The residue was purified by column chromatography (SiO₂, Petroleum ether/Ethyl acetate=1/0 to 5/1) to give tert-butyl N-(8-hydroxy-3-oxo-4H-1,4-benzoxazin-6-yl)carbamate (300 mg, crude) as yellow solid.

### Step 8. tert-butyl N-(8-hydroxy-3,4-dihydro-2H-1,4-benzoxazin-6-yl)carbamate

To a solution of tert-butyl N-(8-hydroxy-3-oxo-4H-1,4-benzoxazin-6-yl)carbamate (300 mg, 1.07 mmol) in THF (4 mL) was added borane tetrahydrofuran (1 M, 2.68 mL) at 0° C., then the mixture was stirred at 20° C. for 2 hrs under N2. LCMS showed the reaction was complete and the desired ms was detected. The reaction was filtered and concentrated under reduced pressure to give tert-butyl N-(8-hydroxy-3,4-dihydro-2H-1,4-benzoxazin-6-yl)carbamate (220 mg, crude) as black solid.

### Step 8. tert-butyl N-(4-benzyl-8-hydroxy-2,3-dihydro-1,4-benzoxazin-6-yl)carbamate

To the mixture of tert-butyl N-(8-hydroxy-3,4-dihydro-2H-1,4-benzoxazin-6-yl)carbamate (220 mg, 826.16 mol) and benzaldehyde (131.51 mg, 1.24 mmol) in MeOH (4 mL) was added CH₃COOH (0.1 mL) and sodium cyanoboranuide (259.59 mg, 4.13 mmol), then the mixture was stirred at 20° C. for 12 hrs. LCMS showed the reaction was complete and the desired ms was detected. The residue was purified by prep-TLC (SiO₂, Petroleum ether/Ethyl acetate=2:1) to give tert-butyl N-(4-benzyl-8-hydroxy-2,3-dihydro-1,4-benzoxazin-6-yl)carbamate (70 mg, crude) as brown oil.

### Step 9. tert-butyl N-[4-benzyl-8-(3-chloropropoxy)-2,3-dihydro-1,4-benzoxazin-6-yl]carbamate

To a solution of tert-butyl N-(4-benzyl-8-hydroxy-2,3-dihydro-1,4-benzoxazin-6-yl)carbamate (70 mg, 196.40 mol) and 1-chloro-3-iodo-propane (44.17 mg, 216.04 μmol, 23.20 μL) in CH₃CN (3 mL) was added dicesium;carbonate (191.97 mg, 589.20 mol), then the mixture was stirred at 50° C. for 2 hrs. LCMS showed the reaction was complete and the desired ms was detected. The reaction was filtered and concentrated under reduced pressure to give tert-butyl N-[4-benzyl-8-(3-chloropropoxy)-2,3-dihydro-1,4-benzoxazin-6-yl]carbamate (80 mg, crude) as yellow solid.

### Step 10. tert-butyl N-[4-benzyl-8-(3-pyrrolidin-1-yl]propoxy)-2,3-dihydro-1,4-benzoxazin-6-yl]carbamate

To a solution of tert-butyl N-[4-benzyl-8-(3-chloropropoxy)-2,3-dihydro-1,4-benzoxazin-6-yl]carbamate (80 mg, 184.78 μmol) and pyrrolidine (65.71 mg, 923.92 μmol, 76.76 μL) in CH₃CN (3 mL) was added dicesium;carbonate (180.62 mg, 554.35 μmol) and iodosodium (55.40 mg, 369.57 μmol, 15.11 μL), then the mixture was stirred at 70° C. for 12 hrs. LCMS showed the reaction was complete and the desired ms was detected. The reaction was filtered and concentrated under reduced pressure to give tert-butyl N-[4-benzyl-8-(3-pyrrolidin-1-ylpropoxy)-2,3-dihydro-1,4-benzoxazin-6-yl]carbamate (90 mg, crude) as purple solid.

### Step 11. 4-benzyl-8-(3-pyrrolidin-1-ylpropoxy)-2,3-dihydro-1,4-benzoxazin-6-amine

To a solution of tert-butyl N-[4-benzyl-8-(3-pyrrolidin-1-ylpropoxy)-2,3-dihydro-1,4-benzoxazin-6-yl]carbamate (90 mg, 192.47 μmol) in DCM (1.20 mL) was added 2,2,2-trifluoroacetic acid (1.78 g, 15.58 mmol, 1.20 mL), then the mixture was stirred at 20° C. for 2 hrs. LCMS showed the reaction was complete and the desired ms was detected. The reaction was concentrated under reduced pressure to give 4-benzyl-8-(3-pyrrolidin-1-ylpropoxy)-2,3-dihydro-1,4-benzoxazin-6-amine (90 mg, crude, TFA) as brown solid.

### Step 12. N2-[4-benzyl-8-(3-pyrrolidin-1-ylpropoxy)-2,3-dihydro-1,4-benzoxazin-6-yl]-N4,6-dimethyl-pyrimidine-2,4-diamine

To the mixture of 4-benzyl-8-(3-pyrrolidin-1-ylpropoxy)-2,3-dihydro-1,4-benzoxazin-6-amine (30 mg, 81.64 μmol) and 2-chloro-N,6-dimethyl-pyrimidin-4-amine (12.87 mg, 81.64 μmol) in i-PrOH (3 mL) was added HCl (12 M, 230.77 μL), then the mixture was stirred at 120° C. in the microwave for 1 h. LCMS showed the reaction was complete and the desired ms was detected. The reaction was filtered and concentrated under reduced pressure to give a residue. The crude product was purified by prep-HPLC (TFA condition:column:Phenomenex luna C18 100*40 mm*5 um; mobile phase:water (0.1% TFA)-ACN; B %: 10%-40%,8 min) to give N2-[4-benzyl-8-(3-pyrrolidin-1-ylpropoxy)-2,3-dihydro-1,4-benzoxazin-6-yl]-N4,6-dimethyl-pyrimidine-2,4-diamine (6.1 mg, 10.14 μmol, 12.42% yield, TFA) as brown solid.

¹H NMR (400 MHz, METHANOL-d₄) 6=7.33-7.24 (m, 5H), 6.82-6.77 (m, 1H), 6.66-6.59 (m, 1H), 5.93-5.88 (m, 1H), 4.54-4.50 (m, 2H), 4.30-4.26 (m, 2H), 4.18 (br t, J=5.2 Hz, 2H), 3.86-3.78 (m, 2H), 3.49-3.43 (m, 4H), 3.18-3.11 (m, 2H), 2.86-2.77 (m, 3H), 2.28-2.18 (m, 7H), 2.10-2.03 (m, 2H). MS (ESI): m/z=489.2 [M+H]

### EXAMPLE 22. Synthesis of Compound 128

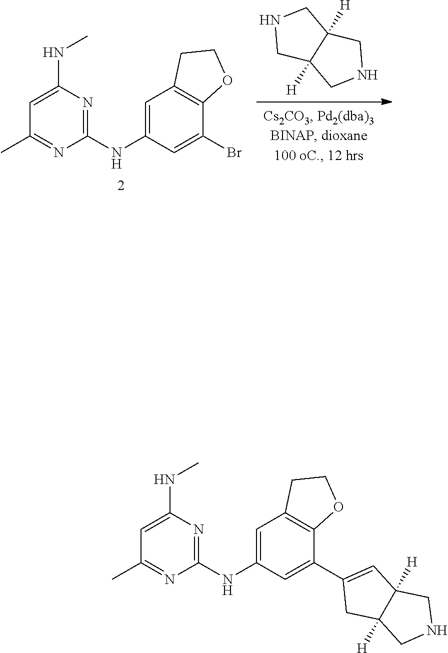 [SMILES: CNc1cc(C)nc(Nc2cc(Br)c3c(c2)CCO3)n1 ; C1NC[C@H]2CNC[C@@H]12 ; CNc1cc(C)nc(Nc2cc3c(c(C4=C[C@H]5CNC[C@H]5C4)c2)OCC3)n1]

### Step 1. N4,6-dimethyl-N2-[7-[rac-(3aS,6aR)-2,3,3a, 4,6,6a-hexahydro-1H-pyrrolo[3,4-c]pyrrol-5-yl]-2,3-dihydrobenzofuran-5-yl pyrimidine-2,4-diamine

To a solution of 1,2,3,3a,4,5,6,6a-octahydropyrrolo[3,4-c]pyrrole (16.73 mg, 149.17 μmol), N2-(7-bromo-2,3-dihydrobenzofuran-5-yl)-N4,6-dimethyl-pyrimidine-2,4-diamine(50 mg, 149.17 μmol), BINAP (18.58 mg, 29.83 μmol), t-BuOK (50.21 mg, 447.50 μmol) in dioxane (1 mL) was added Pd₂(dba)₃ (13.66 mg, 14.92 mol) under N2. The reaction mixture was stirred at 110° C. for 12 hs. LCMS showed starting material was consumed and mass of the desired compound. It was concentrated under reduced pressure then purified by prep-HPLC (TFA condition, column: Phenomenex Luna 80*30 mm*3 um; mobile phase: [water(0.1% TFA)-ACN]; B %: 5%-35%,8 min) to give N4,6-dimethyl-N2-[7-[rac-(3aS,6aR)-2,3,3a,4,6,6a-hexahydro-1H-pyrrolo[3,4-c]pyrrol-5-yl]-2,3-dihydrobenzofuran-5-yl]pyrimidine-2,4-diamine (11.5 mg, 31.38 μmol, 21.04% yield) (100.0% purity, TFA salt) as a white solid.

¹H NMR (400 MHz, METHANOL-d₄) δ ppm 7.01 (s, 1H) 6.78 (br s, 1H) 5.94 (s, 1H) 4.59 (t, J=8.76 Hz, 2H) 3.57-3.65 (m, 4H) 3.11-3.24 (m, 6H) 3.03 (br dd, J=9.69, 5.57 Hz, 2H) 2.98 (s, 3H) 2.27 (s, 3H)

### EXAMPLE 23. Synthesis of Compounds 130 and 131

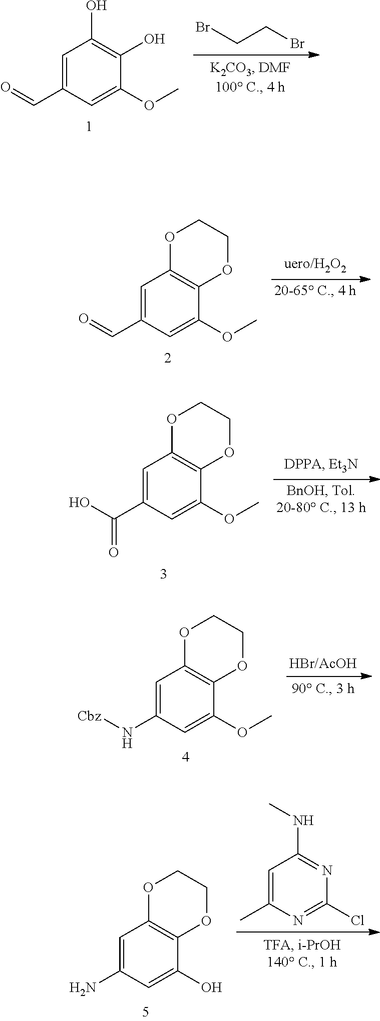

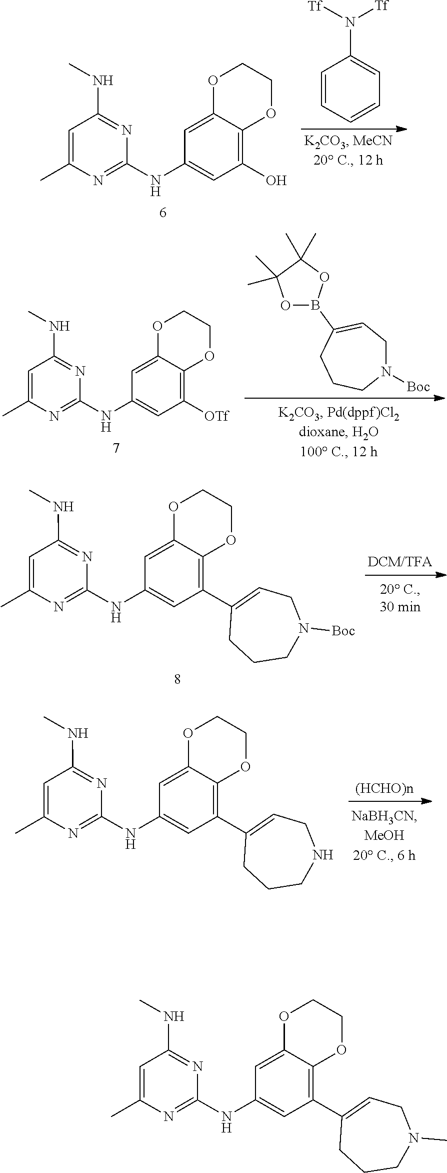

### Step 1. 5-methoxy-2,3-dihydro-1,4-benzodioxine-7-carbaldehyde

To a solution of 3,4-dihydroxy-5-methoxy-benzaldehyde (4 g, 23.79 mmol) in DMF (60 mL) was added 1,2-dibromoethane (4.92 g, 26.17 mmol, 2.25 mL) and K₂CO₃ (6.58 g, 47.58 mmol). It was stirred at 100° C. for 4 h. TLC (petroleum ether:ethyl acetate=1:1, Rf=0.5) indicated starting material was consumed completely, and one major new spot was detected. LCMS showed starting material was consumed completely and mass of the desired compound. The reaction was added into water (200 mL), then extracted with ethyl acetate (3×200 mL). The combined organic layers were washed with brine (3×200 mL) and dried over anhydrous sodium sulfate, filtered and concentrated under reduced pressure to give 5-methoxy-2,3-dihydro-1,4-benzodioxine-7-carbaldehyde (3.9 g, crude) as a light-yellow solid.

### Step 2. 5-methoxy-2,3-dihydro-1,4-benzodioxine-7-carboxylic acid

Aqueous NaOH (6 M, 5.4 mL) was added dropwise to a stirred solution of the hydrogen peroxide urea (25 g, 265.76 mmol) and aldehyde 5-methoxy-2,3-dihydro-1,4-benzodioxine-7-carbaldehyde (3 g, 15.45 mmol) in MeOH (60 mL) at 20° C. The resulting mixture was stirred at 65° C. for 1 h. LCMS showed starting material was consumed completely and mass of the desired compound. The reaction was added saturated sodium thiosulfate aqueous solution (20 mL) at 0° C., then the mixture was stirred at 20° C. for 15 min. The reaction was added saturated sodium sulfite solution (150 mL) and stirred at 20° C. for 15 min, then concentrated under reduced pressure to dry MeOH, then was added 1 N HCl adjust to pH=5-6, filtered and the filter cake was concentrated under reduced pressure to give 5-methoxy-2,3-dihydro-1,4-benzodioxine-7-carboxylic acid (2.7 g, 12.85 mmol, 83.15% yield) as a white solid. ¹H NMR (400 MHz, METHANOL-d₄) δ ppm 3.86 (s, 3H) 4.29 (m, 4H) 7.20 (m, 2H)

### Step 3. benzyl N-(5-methoxy-2,3-dihydro-1,4-benzodioxin-7-yl)carbamate

To a solution of 5-methoxy-2,3-dihydro-1,4-benzodioxine-7-carboxylic acid (2.7 g, 12.85 mmol) in Tol. (30 mL) was added DPPA (3.75 g, 15.42 mmol), TEA (3.90 g, 38.54 mmol, 5.37 mL). It was stirred at 20° C. for 1 h. Then it was added phenylmethanol (4.17 g, 38.54 mmol, 3.99 mL) It was stirred at 80° C. for 12 h. TLC (petroleum ether:ethyl acetate=3:1, Rf=0.2) indicated starting material was consumed completely, and two major new spots were detected. LCMS showed starting material was consumed completely and mass of the desired compound. The reaction was added water (50 mL), then extracted with ethyl acetate (3×50 mL) and dried over anhydrous sodium sulfate, filtered and concentrated under reduced pressure to give a residue. The residue was purified by flash chromatography (silica gel, petroleum ether/ethyl acetate=1/0 to 1/1) to give crude product. The crude product was triturated with petroleum ether (50 mL) at 20° C. for 20 min. The reaction was filtered, then the filter cake was concentrated under reduced pressure to give benzyl N-(5-methoxy-2,3-dihydro-1,4-benzodioxin-7-yl)carbamate (2 g, 6.34 mmol, 57.14% yield) as an off-white solid. ¹H NMR (400 MHz, METHANOL-d₄) δ ppm 3.77 (s, 3H) 4.20 (s, 4H) 5.14 (s, 2H) 6.62 (br s, 1H) 6.71 (br s, 1H) 7.25 (m, 2H) 7.39 (m, 3H)

### Step 4. 7-amino-2,3-dihydro-1,4-benzodioxin-5-ol

A solution of benzyl N-(5-methoxy-2,3-dihydro-1,4-benzodioxin-7-yl)carbamate (600 mg, 1.90 mmol) in HBr/AcOH (5 mL), then the mixture was stirred at 90° C. for 3 h. TLC (petroleum ether:ethyl acetate=1:1, Rf=0.3) indicated starting material was consumed completely, and one major new spot was detected. LCMS showed starting material was consumed completely and mass of the desired compound. The reaction was added water (10 mL), then extracted with dichloromethane (20 mL). Then the aqueous phase was added sodium hydrogen carbonate adjust to pH=7-8, then extracted with ethyl acetate (3×20 mL) and dried over anhydrous sodium sulfate, filtered and concentrated under reduced pressure to give 7-amino-2,3-dihydro-1,4-benzodioxin-5-ol (490 mg, crude) as a yellow solid. ¹H NMR (400 MHz, METHANOL-d₄) δ ppm 4.17 (s, 4H) 5.82 (d, J=2.63 Hz, 1H) 5.91 (m, 1H)

### Step 5. 7-[[4-methyl-6-(methylamino)pyrimidin-2-yl]amino]-2,3-dihydro-1,4-benzodioxin-5-ol

To a solution of 7-amino-2,3-dihydro-1,4-benzodioxin-5-ol (100 mg, 598.22 μmol) and 2-chloro-N,6-dimethyl-pyrimidin-4-amine (94.28 mg, 598.22 μmol) in i-PrOH (2 mL) was added TFA (6.82 mg, 59.82 μmol, 4.61 μL), then the mixture was stirred at 140° C. for 1 h. TLC (petroleum ether:ethyl acetate=0:1, Rf=0.3) indicated Reactant 1 was consumed completely, and one major new spot was detected. LCMS showed starting material was consumed completely and mass of the desired compound. The reaction was filtered and the filter cake concentrated under reduced pressure to give 7-[[4-methyl-6-(methylamino)pyrimidin-2-yl]amino]-2,3-dihydro-1,4-benzodioxin-5-ol (170 mg, crude) as a white solid.

### Step 6. [7-[[4-methyl-6-(methylamino)pyrimidin-2-yl]amino]-2,3-dihydro-1,4-benzodioxin-5-yl]trifluoromethanesulfonate

To a solution of 7-[[4-methyl-6-(methylamino)pyrimidin-2-yl]amino]-2,3-dihydro-1,4-benzodioxin-5-ol (170 mg, 589.66 μmol) and 1,1,1-trifluoro-N-phenyl-N-(trifluoromethylsulfonyl)methanesulfonamide (252.79 mg, 707.59 μmol) in MeCN (5 mL) was added K₂CO₃ (407.47 mg, 2.95 mmol), then the mixture was stirred at 20° C. for 12 h. TLC (petroleum ether:ethyl acetate=0:1, Rf=0.9) indicated Reactant 1 was consumed completely, and one major new spot was detected. LCMS showed starting material was consumed completely and mass of the desired compound. The reaction was filtered and concentrated under reduced pressure to give a residue. The residue was purified by prep-TLC (SiO₂, petroleum ether:ethyl acetate=0:1) to give [7-[[4-methyl-6-(methylamino)pyrimidin-2-yl]amino]-2,3-dihydro-1,4-benzodioxin-5-yl]trifluoromethanesulfonate (250 mg, crude) as a light-yellow solid.

### Step 7. tert-butyl 5-[7-[[4-methyl-6-(methylamino)pyrimidin-2-yl]amino]-2,3-dihydro-1,4-benzodioxin-5-yl]-2,3,4,7-tetrahydroazepine-1-carboxylate

To a solution of [7-[[4-methyl-6-(methylamino)pyrimidin-2-yl]amino]-2,3-dihydro-1,4-benzodioxin-5-yl]trifluoromethanesulfonate (180 mg, 428.20 μmol), tert-butyl 5-(4,4,5,5-tetramethyl-1,3,2-dioxaborolan-2-yl)-2,3,4,7-tetrahydroazepine-1-carboxylate (346.02 mg, 1.07 mmol), K₂CO₃ (118.36 mg, 856.40 μmol) and H₂O (0.5 mL) in dioxane (5 mL) was added cyclopentyl(diphenyl)phosphane dichloropalladium iron (31.33 mg, 42.82 μmol), then the mixture was stirred at 100° C. for 12 h under an atmosphere of nitrogen. TLC (petroleum ether:ethyl acetate=1:1, Rf=0.5) indicated Reactant 1 was consumed completely, and one major new spot was detected. LCMS showed starting material was consumed completely and mass of the desired compound. The reaction was filtered and concentrated under reduced pressure to give a residue. The residue was purified by prep-TLC (SiO₂, petroleum ether:ethyl acetate=1:1) to give tert-butyl 5-[7-[[4-methyl-6-(methylamino)pyrimidin-2-yl]amino]-2,3-dihydro-1,4-benzodioxin-5-yl]-2,3,4,7-tetrahydroazepine-1-carboxylate (200 mg, crude) as a brown solid.

### Step 8. N4,6-dimethyl-N2-[5-(2,3,4,7-tetrahydro-1H-azepin-5-yl]-2,3-dihydro-1,4-benzodioxin-1-yl]pyrimidine-2,4-diamine

To a solution of tert-butyl 5-[7-[[4-methyl-6-(methylamino)pyrimidin-2-yl]amino]-2,3-dihydro-1,4-benzodioxin-5-yl]-2,3,4,7-tetrahydroazepine-1-carboxylate (200 mg, 427.75 μmol) in DCM (2 mL) was added TFA (1 mL), then the mixture was stirred at 20° C. for 30 min. LCMS showed starting material was consumed completely and mass of the desired compound. The reaction was filtered and concentrated under reduced pressure to give N4,6-dimethyl-N2-[5-(2,3,4,7-tetrahydro-1H-azepin-5-yl)-2,3-dihydro-1,4-benzodioxin-7-yl]pyrimidine-2,4-diamine (100 mg, crude) as a brown oil. Product 2 was purified by prep-HPLC (TFA condition, column: Phenomenex luna C¹⁸ 80*40 mm*3 um; mobile phase: [water(0.1% TFA)-ACN]; B %: 10%-28%, 7 min) to give N4,6-dimethyl-N2-[5-(2,3,4,7-tetrahydro-1H-azepin-5-yl)-2,3-dihydro-1,4-benzodioxin-7-yl]pyrimidine-2,4-diamine (25.4 mg, 52.87 μmol, 12.36% yield, TFA) (purity: 100%) as a light-yellow solid. Product 2 was rechecked by LCMS and HNMR.

¹H NMR (400 MHz, METHANOL-d₄) δ ppm 2.06 (m, 2H) 2.28 (s, 3H) 2.76 (m, 2H) 2.99 (s, 3H) 3.47 (m, 2H) 3.87 (d, J=6.36 Hz, 2H) 4.28 (s, 4H) 5.90 (t, J=6.36 Hz, 1H) 5.96 (s, 1H) 6.85 (d, J=1.83 Hz, 1H) 7.28 (br s, 1H).

### Step 9. N4,6-dimethyl-N2-[5-(1-methyl-2,3,4,7-tetrahydroazepin-5-yl]-2,3-dihydro-1,4-benzodioxin-7-yl]pyrimidine-2,4-diamine

To a solution of N4,6-dimethyl-N2-[5-(2,3,4,7-tetrahydro-1H-azepin-5-yl)-2,3-dihydro-1,4-benzodioxin-7-yl]pyrimidine-2,4-diamine (100 mg, 272.15 μmol) in MeOH (2 mL) was added DIEA adjust to pH=7-8, then the mixture was added (HCHO)n (81.72 mg, 2.72 mmol) and AcOH adjust to pH=5-6. Then the mixture was stirred at 20° C. for 15 min. Then the mixture was added NaBH₃CN (34.20 mg, 544.30 μmol), then the reaction was stirred at 20° C. for 6 h. LCMS showed starting material was consumed completely and mass of the desired compound. The reaction was filtered and concentrated under reduced pressure to give a residue. The residue was purified by prep-HPLC (TFA condition, column: Phenomenex luna C¹⁸ 100*40 mm*5 um; mobile phase: [water(0.1% TFA)-ACN]; B %: 1%-25%, 8 min) to give N4,6-dimethyl-N2-[5-(1-methyl-2,3,4,7-tetrahydroazepin-5-yl)-2,3-dihydro-1,4-benzodioxin-7-yl]pyrimidine-2,4-diamine (45.5 mg, 92.01 μmol, 33.81% yield, TFA) (purity: 100%) as a white solid.

¹H NMR (400 MHz, METHANOL-d₄) δ ppm 2.11 (br s, 2H) 2.28 (s, 3H) 2.77 (br d, J=5.75 Hz, 2H) 2.93 (s, 3H) 2.99 (s, 3H) 3.46 (m, 1H) 3.67 (m, 1H) 4.00 (m, 2H) 4.28 (s, 4H) 5.84 (br t, J=6.36 Hz, 1H) 5.97 (s, 1H) 6.85 (br s, 1H) 7.27 (s, 1H).

### EXAMPLE 24. Synthesis of Compound 133

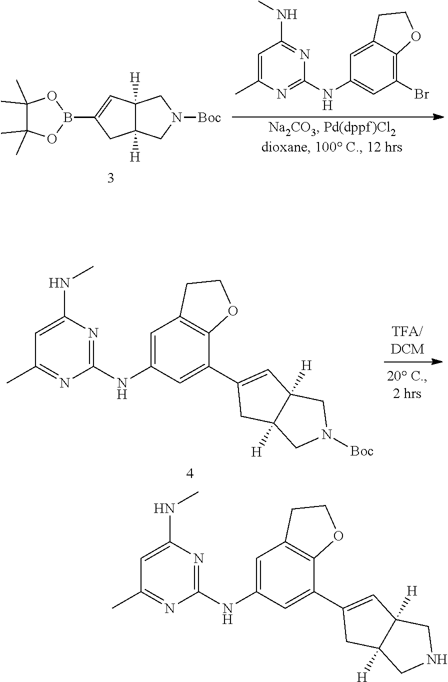

### Step 1. tert-butyl 5-[5-[[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate

To a solution of tert-butyl 5-(4,4,5,5-tetramethyl-1,3,2-dioxaborolan-2-yl)-3,3a,6,6a-tetrahydro-1H-cyclopenta[c]pyrrole-2-carboxylate (110.02 mg, 328.16 μmol) in dioxane (2 mL) and H₂O (0.2 mL) was added N2-(7-bromo-2,3-dihydrobenzofuran-5-yl)-N4,6-dimethyl-pyrimidine-2,4-diamine (50 mg, 149.17 μmol) and Na₂CO₃ (31.62 mg, 298.33 mol, 12.49 μL), then it was added Pd(dppf)Cl₂ (10.91 mg, 14.92 μmol) under N₂ atmosphere. It was stirred at 100° C. for 12 hours under N₂ atmosphere. LCMS showed starting material was consumed completely and mass of the desired compound. Then it was concentrated under reduced pressure to give tert-butyl 5-[5-[[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate (69 mg, crude) as a black solid.

### Step 2. N2-[7-(1,2,3,3a,6,6a-hexahydrocyclopenta[c]pyrrol-5-yl]-2,3-dihydrobenzo furan-5-yl]-N4,6-dimethyl-pyrimidine-2,4-diamine

To a solution of tert-butyl 5-[5-[[4-methyl-6-(methylamino) pyrimidin-2-yl]amino]-2,3-dihydrobenzofuran-7-yl]-3,3a,6,6a-tetrahydro-1H-cyclopenta[c]pyrrole-2-carboxylate (69 mg, 148.84 μmol) in DCM (2 mL) and TFA (1 mL) was stirred at 20° C. for 12 hours. LCMS showed starting material was consumed completely and mass of the desired compound. Then it was concentrated under reduced pressure to give a residue. It was purified by prep-HPLC (TFA condition, column: Phenomenex luna C18 100*40 mm*3 um; mobile phase: [water(TFA)-ACN]; B %: 1%-45%, 8 min,) to give N2-[7-(1,2,3,3a,6,6a-hexahydrocyclopenta [c]pyrrol-5-yl)-2,3-dihydrobenzofuran-5-yl]-N4,6-dimethyl-pyrimidine-2,4-diamine (27 mg, 74.29 μmol, 49.91% yield) (100% purity, TFA salt) as a white solid.

¹H NMR (400 MHz, METHANOL-d₄) δ ppm 2.30 (s, 3H) 2.80 (br d, J=14.63 Hz, 1H) 2.98 (s, 3H) 3.09-3.20 (m, 2H) 3.23 (br d, J=7.63 Hz, 1H) 3.28 (br t, J=8.82 Hz, 2H) 3.35-3.39 (m, 1H) 3.37 (br d, J=2.63 Hz, 1H) 3.40-3.47 (m, 1H) 3.53 (dd, J=11.57, 8.57 Hz, 1H) 3.83 (br s, 1H) 4.64-4.72 (m, 2H) 5.97 (s, 1H) 6.44 (s, 1H) 7.26 (s, 1H) 7.36 (s, 1H).

MS (ESI): m/z=364.3 [M+H]⁺.

### EXAMPLE 25. Synthesis of Compound 134

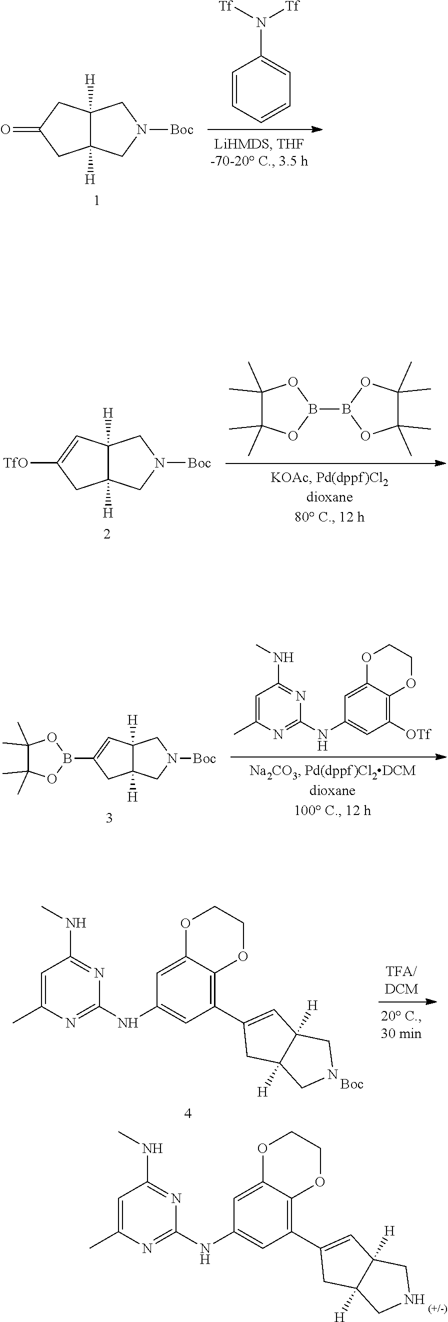

### Step 1. Tert-butyl5-(trifluoromethylsulfonyloxy)-3,3a,6,6a-tetrahydro-1H-cyclopenta[c]pyrrole-2-carboxylate

To a solution of tert-butyl 5-oxo-1,3,3a,4,6,6a-hexahydrocyclopenta[c]pyrrole-2-carboxylate (200 mg, 887.77 μmol) in THF (2 mL) was added lithium bis(trimethylsilyl) amide (1 M, 1.15 mL) at −70° C. under an atmosphere of nitrogen, then the mixture was stirred at −70° C. for 30 min under an atmosphere of nitrogen. Then the mixture was added dropwise a solution of 1,1,1-trifluoro-N-phenyl-N-(trifluoromethylsulfonyl)methanesulfonamide (444.02 mg, 1.24 mmol) in 1,1,1-trifluoro-N-phenyl-N-(trifluoromethylsulfonyl)methanesulfonamide (444.02 mg, 1.24 mmol) with stirring at −70° C. for 1 h under an atmosphere of nitrogen, then the mixture was stirred at 20° C. for 2 h under an atmosphere of nitrogen. TLC (petroleum ether:ethyl acetate=5:1, Rf=0.5) (KMnO₄ color developing agent) indicated Reactant 1 was consumed completely, and two major new spots were detected. The reaction was added into water (5 mL), then extracted with ethyl acetate (3×10 mL). The combined organic layers were washed with saturated ammonium chloride solution (3×10 mL) and dried over anhydrous sodium sulfate, filtered and concentrated under reduced pressure to give a residue. The residue was purified by prep-TLC (SiO₂, petroleum ether:ethyl acetate=3:1) to give tert-butyl 5-(trifluoromethylsulfonyloxy)-3,3a,6,6a-tetrahydro-1H-cyclopenta[c]pyrrole-2-carboxylate (280 mg, 783.56 μmol, 88.26% yield) as a light-yellow oil.

### Step 2. Tert-butyl 5-(4,4,5,5-tetramethyl-]3,2-dioxaborolan-2-yl)-3,3a,6,6a-tetrahydro-1H-cyclopenta[c]pyrrole-2-carboxylate

To a solution of tert-butyl 5-(trifluoromethylsulfonyloxy)-3,3a,6,6a-tetrahydro-1H-cyclopenta[c]pyrrole-2-carboxylate (70 mg, 195.89 μmol), 4,4,4′,4′,5,5,5′,5′-octamethyl-2,2′-bi(1,3,2-dioxaborolane) (84.56 mg, 333.01 μmol) and potassium acetate (57.67 mg, 587.67 μmol, 36.73 μL) in dioxane (2 mL) was added cyclopentyl(diphenyl)phosphane;dichloromethane;dichloropalladium;iron (16.00 mg, 19.59 μmol) under an atmosphere of nitrogen, then the mixture was stirred at 100° C. for 12 h under an atmosphere of nitrogen. TLC (petroleum ether:ethyl acetate=5:1, Rf=0.6) indicated Reactant 1 was consumed completely, and one major new spot was detected. LCMS showed mass of the desired compound. The reaction was filtered and concentrated under reduced pressure to give tert-butyl 5-(4,4,5,5-tetramethyl-1,3,2-dioxaborolan-2-yl)-3,3a,6,6a-tetrahydro-1H-cyclopenta[c]pyrrole-2-carboxylate (65 mg, crude) as a brown oil.

### Step 3. Tert-butyl 5-[7-[[4-methyl-6-(methylamino)pyrimidin-2-yl]amino]-2,3-dihydro-1,4-benzodioxin-5-yl]-3,3a,6,6a-tetrahydro-1H-cyclopenta[c]pyrrole-2-carboxylate

To a solution of tert-butyl 5-(4,4,5,5-tetramethyl-1,3,2-dioxaborolan-2-yl)-3,3a,6,6a-tetrahydro-1H-cyclopenta[c]pyrrole-2-carboxylate (40 mg, 119.32 μmol), [7-[[4-methyl-6-(methylamino)pyrimidin-2-yl]amino]-2,3-dihydro-1,4-benzodioxin-5-yl]trifluoromethanesulfonate (50.16 mg, 119.32 μmol), Na₂CO₃ (25.29 mg, 238.63 μmol) and H₂O (0.1 mL) in dioxane (1 mL) was added cyclopentyl(diphenyl)phosphane dichloropalladium iron (8.73 mg, 11.93 μmol), then the mixture was stirred at 100° C. for 4 h under an atmosphere of nitrogen. TLC (petroleum ether:ethyl acetate=1:1, Rf=0.4) indicated Reactant 1 was consumed completely, and one major new spot was detected. LCMS showed starting material was consumed completely and mass of the desired compound. The reaction was filtered and concentrated under reduced pressure to give a residue. The residue was purified by prep-TLC (SiO₂, petroleum ether:ethyl acetate=1:1) to give tert-butyl 5-[7-[[4-methyl-6-(methylamino)pyrimidin-2-yl]amino]-2,3-dihydro-1,4-benzodioxin-5-yl]-3,3a,6,6a-tetrahydro-1H-cyclopenta[c]pyrrole-2-carboxylate (60 mg, crude) as a brown oil.

### Step 4. N2-[5-(1,2,3,3a,6,6a-hexahydrocyclopenta[c]pyrrol-5-yl]-2,3-dihydro-1,4-benzodioxin-7-yl]-N4,6-dimethyl-pyrimidine-2,4-diamine

To a solution of tert-butyl 5-[7-[[4-methyl-6-(methylamino)pyrimidin-2-yl]amino]-2,3-dihydro-1,4-benzodioxin-5-yl]-3,3a,6,6a-tetrahydro-1H-cyclopenta[c]pyrrole-2-carboxylate (60 mg, 125.11 μmol) in DCM (1 mL) was added TFA (0.5 mL), then the mixture was stirred at 20° C. for 30 min. LCMS showed starting material was consumed completely and mass of the desired compound. The reaction was filtered and concentrated under reduced pressure to give a residue. The residue was purified by prep-HPLC (TFA condition, column: Phenomenex luna C¹⁸ 80*40 mm*3 um; mobile phase: [water(0.1% TFA)-ACN]; B %: 12%-28%,7 min) to give N2-[5-(1,2,3,3a,6,6a-hexahydrocyclopenta[c]pyrrol-5-yl)-2,3-dihydro-1,4-benzodioxin-7-yl]-N4,6-dimethyl-pyrimidine-2,4-diamine (8.5 mg, 17.26 μmol, 13.80% yield, TFA) (purity: 100%) as a light-yellow solid. It was rechecked by LCMS and HNMR.

¹H NMR (400 MHz, METHANOL-d₄) δ ppm 2.28 (s, 3H) 2.77 (br d, J=13.81 Hz, 1H) 2.99 (s, 3H) 3.10 (br dd, J=11.55, 4.71 Hz, 1H) 3.17 (m, 2H) 3.35 (m, 1H) 3.48 (m, 2H) 3.79 (m, 1H) 4.30 (m, 4H) 5.97 (s, 1H) 6.33 (s, 1H) 7.01 (d, J=2.32 Hz, 1H) 7.20 (s, 1H)

### EXAMPLE 26. Synthesis of Compound 136

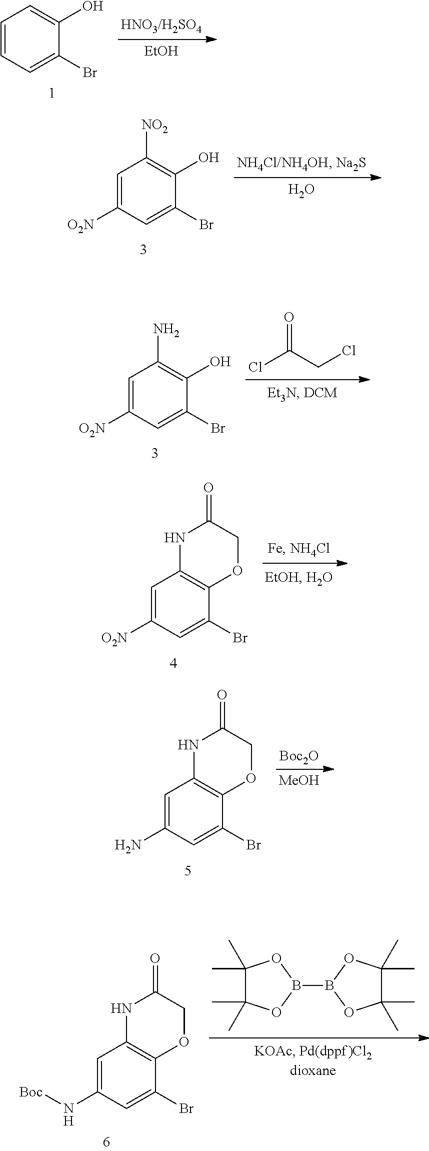

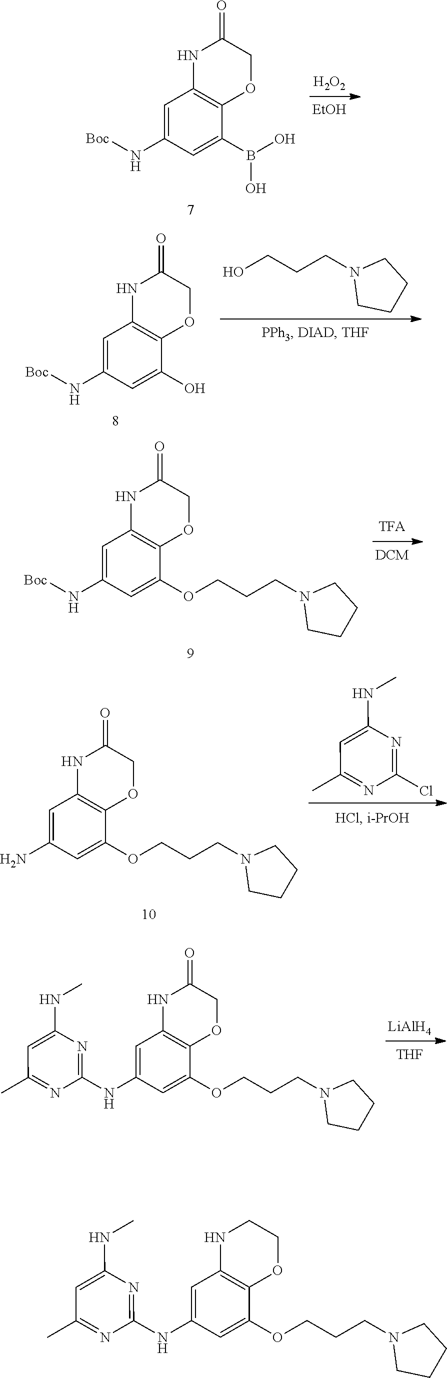

### Step 1. 2-bromo-4,6-dinitro-phenol

2-Bromophenol (10 g, 57.80 mmol, 6.70 mL) was dissolved in EtOH (50 mL) and H₂SO₄ (100 mL), and then to the mixture was added HNO₃ (30.00 g, 476.09 mmol, 20 mL) slowly, and then the mixture was stirred at 25° C. for 12 h. TLC (petroleum ether:ethyl acetate=0:1, Rf=0.5) indicated Reactant 1 was consumed completely, and one major new spot was detected. The mixture was added to H₂O (500 mL) at 5° C., and then the suspension was filtered, and washed with H₂O (50 mL*2), and the filtered cake was the desired compound, and the filtrate was quenched by sat. 2M NaOH to pH=7 at 25° C. slowly. 2-bromo-4,6-dinitro-phenol (5 g, crude) was obtained as a yellow solid.

### Step 2. 2-amino-6-bromo-4-nitro-phenol

NH₄Cl (10.00 g, 186.95 mmol) and NH₄OH (2.67 g, 19.01 mmol, 2 mL, 25% purity) were added to a solution of 2-bromo-4,6-dinitro-phenol (5 g, 19.01 mmol) in H₂O (50 mL). The mixture was heated to 80° C. sodiosulfanylsodium nonahydrate (5.50 g, 22.90 mmol) was added, After addition, reaction was heated for 2 h at 80° C. LCMS showed reactant 1 was consumed completely and desired MS was detected. TLC (petroleum ether:ethyl acetate=3:1, Rf=0.1) indicated Reactant 1 was consumed completely, and one major new spot was detected. The mixture was diluted with H₂O (200 mL), and then to the mixture was added AcOH to pH=2 at 10° C., and then extracted with EtOAc(100 mL×3), and then the organic phase was concentrated in vacuo. 2-amino-6-bromo-4-nitro-phenol (2.5 g, crude) was obtained as a black solid.

### Step 3. 8-bromo-6-nitro-4H-1,4-benzoxazin-3-one

2-Amino-6-bromo-4-nitro-phenol (2 g, 8.58 mmol) was dissolved in DCM (30 mL) and then to the mixture was added TEA (2.61 g, 25.75 mmol, 3.59 mL) 2-chloroacetyl chloride (1.16 g, 10.30 mmol, 820.9 μL) and then the mixture was stirred at 25° C. for 12 h. LCMS showed reactant 1 was consumed completely and a main peak was detected. TLC (petroleum ether:ethyl acetate=5:1, Rf=0.1) indicated Reactant 1 was consumed completely, and one major new spot was detected. The reaction was diluted by H₂O (100 mL), and then extracted with DCM (20 mL×2), the organic phase was concentrated in vacuo. 8-bromo-6-nitro-4H-1,4-benzoxazin-3-one (2.3 g, crude) was obtained as a yellow oil.

### Step 4. 6-amino-8-bromo-4H-1,4-benzoxazin-3-one

8-Bromo-6-nitro-4H-1,4-benzoxazin-3-one (2.3 g, 8.42 mmol) was dissolved in H₂O (10 mL) EtOH (40 mL), and then to the mixture was added Fe (4.70 g, 84.24 mmol) NH₄Cl (4.51 g, 84.24 mmol), and then the mixture was stirred at 80° C. for 1 h. LCMS showed reactant 1 was consumed completely and desired MS was detected. The reaction was cooled to 50° C., and the filtered, the filter cake was washed with MeOH (40 mL×2). The filtrate was concentrated in vacuo at 50° C. 6-amino-8-bromo-4H-1,4-benzoxazin-3-one (2 g, crude) was obtained as a black solid.

### Step 5. tert-butyl N-(8-bromo-3-oxo-4H-1,4-benzoxazin-6-yl)carbamate

6-Amino-8-bromo-4H-1,4-benzoxazin-3-one (2.05 g, 8.43 mmol) was dissolved in MeOH (50 mL), and then to the mixture was added tert-butoxycarbonyl tert-butyl carbonate (5.52 g, 25.30 mmol, 5.81 mL), and then the mixture was stirred at 60° C. for 12 h. LCMS showed reactant 1 was consumed completely and desired MS was detected. The mixture was concentrated in vacuo, and the residue was purified by flash chromatography (silica gel, petroleum ether/ethyl acetate=2/1 to 1/1 get the spot, 0/1). tert-butyl N-(8-bromo-3-oxo-4H-1,4-benzoxazin-6-yl) carbamate (2 g, 5.83 mmol, 69.10% yield) was obtained as a yellow solid.

### Step 6. [6-(tert-butoxycarbonylamino)-3-oxo-4H-1,4-benzoxazin-8-yl]boronic acid

Tert-butyl N-(8-bromo-3-oxo-4H-1,4-benzoxazin-6-yl) carbamate (1.8 g, 5.25 mmol) was dissolved in dioxane (30 mL) and the to the mixture was added 4,4,5,5-tetramethyl-2-(4,4,5,5-tetramethyl-1,3,2-dioxaborolan-2-yl)-1,3,2-dioxaborolane (1.60 g, 6.29 mmol) KOAc (2.96 g, 10.49 mmol) cyclopentyl(diphenyl)phosphane;dichloropalladium;iron (383.79 mg, 524.52 μmol), and then the mixture was stirred at 80° C. for 12 h under N2. LCMS showed reactant 1 was consumed completely and desired MS was detected. The mixture was concentrated in vacuo. [6-(tert-butoxycarbonylamino)-3-oxo-4H-1,4-benzoxazin-8-yl]boronic acid (1.62 g, crude) was obtained as a black solid.

### Step 7. tert-butyl N-(8-hydroxy-3-oxo-4H-1,4-benzoxazin-6-yl)carbamate

[6-(Tert-butoxycarbonylamino)-3-oxo-4H-1,4-benzoxazin-8-yl]boronic acid (1.6 g, 5.19 mmol) was dissolved in EtOH (30 mL), and to the mixture was added H₂O₂ (1.18 g, 10.39 mmol, 30% purity) at 0° C., and then the mixture was stirred at 25° C. for 2 h. LCMS showed reactant 1 was consumed completely and desired MS was detected. The mixture was diluted with H₂O (60 mL), and then the mixture was quenched by sat. aq. Na₂SO₃ (50 mL), and then the mixture was extracted with EtOAc (30 mL×3), the organic phase was concentrated in vacuo.

The residue was purified by flash chromatography (silica gel, petroleum ether/ethyl acetate=50/1 to 1/1 get the spot, 0/1). tert-butyl N-(8-hydroxy-3-oxo-4H-1,4-benzoxazin-6-yl)carbamate (1.3 g, 4.64 mmol, 89.31% yield) was obtained as a yellow solid.

### Step 8. tert-butyl N-[3-oxo-8-(3-pyrrolidin-1-ylpropoxy)-4H-1,4-benzoxazin-6-yl]carbamate

Tert-butyl N-(8-hydroxy-3-oxo-4H-1,4-benzoxazin-6-yl)carbamate (220 mg, 784.94 μmol) was dissolved in THF (4 mL), and then to the mixture was added 3-pyrrolidin-1-ylpropan-1-ol (121.70 mg, 941.93 μmol), PPh₃ (308.82 mg, 1.18 mmol), and then to the mixture was added ethyl (NE)-N-ethoxycarbonyliminocarbamate (238.08 mg, 1.37 mmol, 231.82 μL) under N2 at 0° C., then the mixture was stirred at 20° C. for 12 h under N2. LCMS showed the reaction was complete and the desired MS was detected. The reaction was filtered and concentrated under reduced pressure to give a residue. The crude product was purified by prep-HPLC (TFA condition:column:Phenomenex luna C18 100*40 mm*5 um; mobile phase:water (0.1% TFA)-ACN; B %: 5%-38%,8 min) to give tert-butyl N-[3-oxo-8-(3-pyrrolidin-1-ylpropoxy)-4H-1,4-benzoxazin-6-yl]carbamate (120 mg, crude) as white solid.

¹H NMR (400 MHz, METHANOL-d₄) δ ppm 6.85-6.82 (m, 1H), 6.72-6.69 (m, 1H), 4.55-4.50 (m, 2H), 4.17-4.13 (m, 2H), 3.82-3.74 (m, 2H), 3.46-3.41 (m, 2H), 3.14 (br s, 2H), 2.27-2.16 (m, 4H), 2.07-2.00 (m, 2H), 1.52-1.49 (m, 9H).

### Step 9. 6-amino-8-(3-pyrrolidin-1-yl]propoxy)-4H-1,4-benzoxazin-3-one

To a solution of tert-butyl N-[3-oxo-8-(3-pyrrolidin-1-ylpropoxy)-4H-1,4-benzoxazin-6-yl]carbamate (110 mg, 281.00 μmol) in DCM (2 mL) was added 2,2,2-trifluoroacetic acid (296.00 mg, 2.60 mmol, 0.2 mL), then the mixture was stirred at 20° C. for 4 h. LCMS showed the reaction was complete and the desired MS was detected. The reaction was concentrated under reduced pressure to give 6-amino-8-(3-pyrrolidin-1-ylpropoxy)-4H-1,4-benzoxazin-3-one (80 mg, crude) as white solid.

### Step 10. 6-[[4-methyl-6-(methylamino)pyrimidin-2-yl]amino]-8-(3-pyrrolidin-1-ylpropoxy)-4H-1,4-benzoxazin-3-one

To the mixture of 6-amino-8-(3-pyrrolidin-1-ylpropoxy)-4H-1,4-benzoxazin-3-one (20 mg, 68.65 mol) and 2-chloro-N,6-dimethyl-pyrimidin-4-amine (10.82 mg, 68.65 μmol) in i-PrOH (3 mL) was added HCl (12 M, 0.04 mL), then the mixture was stirred at 120° C. in the microwave for 0.5 h. LCMS showed the reaction was complete and the desired MS was detected. The reaction was filtered and concentrated under reduced pressure to give a residue. The crude product was purified by prep-HPLC (TFA condition:Phenomenex luna C18 100*40 mm*5 um; mobile phase:water(0.1% TFA)-ACN; B %: 1%-26%,8 min) to give 6-[[4-methyl-6-(methylamino)pyrimidin-2-yl]amino]-8-(3-pyrrolidin-1-ylpropoxy)-4H-1,4-benzoxazin-3-one (100.2 mg, 190.68 μmol, 69.61% yield, TFA) as white solid.

¹H NMR (400 MHz, METHANOL-d₄) δ ppm 7.09-6.85 (m, 2H), 6.05-5.93 (m, 1H), 4.63-4.56 (m, 2H), 4.23-4.16 (m, 2H), 3.84-3.75 (m, 2H), 3.49-3.42 (m, 2H), 3.17-3.10 (m, 2H), 3.06-2.97 (m, 3H), 2.33-2.24 (m, 5H), 2.23-2.16 (m, 2H), 2.09-2.01 (m, 2H) MS (ESI): m/z=413.1 [M+H]⁺

### Step 11. N4,6-dimethyl-N2-[8-(3-pyrrolidin-1-yl]propoxy)-3,4-dihydro-2H-1,4-benzoxazin-6-yl]pyrimidine-2,4-diamine

To a solution of 6-[[4-methyl-6-(methylamino) pyrimidin-2-yl]amino]-8-(3-pyrrolidin-1-ylpropoxy)-4H-1,4-benzoxazin-3-one (17 mg, 41.21 μmol) in THF (1 mL) was added LiAlH₄ (2.35 mg, 61.82 μmol) at 0° C., then the mixture was stirred at 20° C. for 1 h. LCMS showed desired mass was detected. e reaction mixture was quenched by water (1 mL), he reaction was filtered and concentrated under reduced pressure (20° C.) to give a residue. e crude product was purified by prep-HPLC(TFA condition:Phenomenex luna C18 100*40 mm*5 um; mobile phase:water(TFA)-ACN]; B %: 1%-45%, 8 min) to give N4,6-dimethyl-N2-[8-(3-pyrrolidin-1-ylpropoxy)-3, -dihydro-2H-1,4-benzoxazin-6-yl]pyrimidine-2,4-diamine (3.9 mg, 7.62 μmol, 18.50% yield, TFA) as pale yellow solid.

¹H NMR (400 MHz, METHANOL-d₄) δ ppm 6.59 (br s, 2H), 5.94 (s, 1H), 4.23 (br s, 2H), 4.15 (br t, J=4.9 Hz, 2H), 3.81 (br s, 2H), 3.46 (br t, J=6.5 Hz, 2H), 3.39 (br s, 2H), 3.18-3.09 (m, 2H), 3.00 (s, 3H), 2.32-2.14 (m, 7H), 2.05 (br d, J=5.4 Hz, 2H)

MS (ESI): m/z=399.1M+H]⁺

### EXAMPLE 27. Synthesis of Compounds 137, 141 and 145

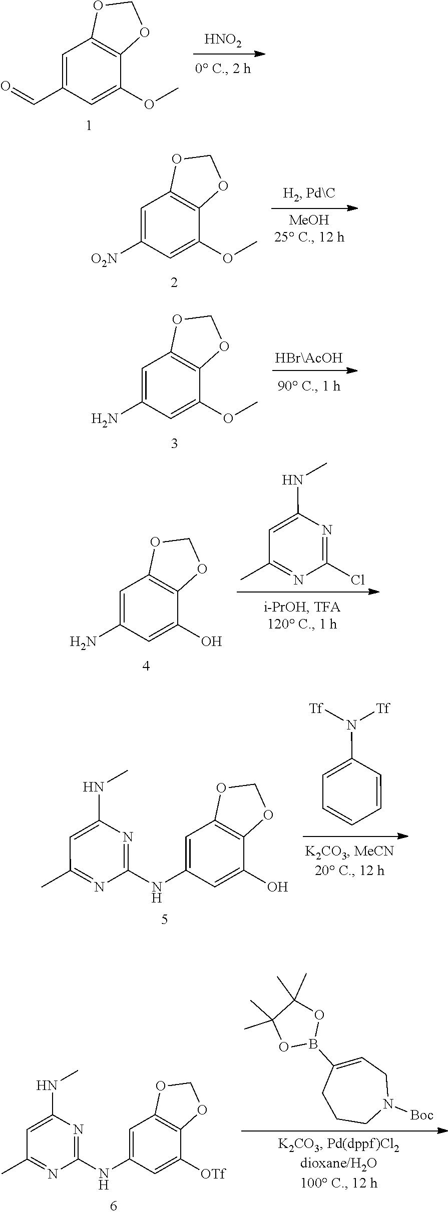

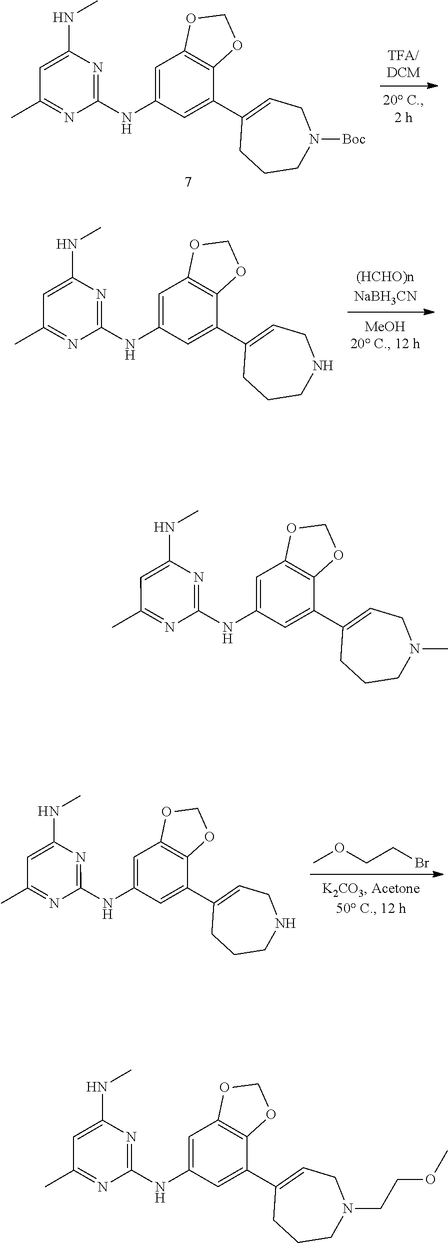

### Step 1. 4-methoxy-6-nitro-1,3-benzodioxole

To a flask containing stirred nitric acid (8.87 g, 140.77 mmol, 6.25 mL) cooled to 0° C. was added 7-methoxy-1,3-benzodioxole-5-carbaldehyde (1 g, 5.55 mmol) portion wise. The reaction was stirred at 0° C. for 2 h. TLC (Petroleum ether:Ethyl acetate=4:1, Rf=0.45) showed the starting material was consumed and new spot was formed. The five reactions were work up together. The reaction mixture was quenched by water (50 mL), then the mixture was filtered to give a residue. The residue was purified by column Petroleum ether/Ethyl acetate=15/1 to 1/1) to give 4-methoxy-6-nitro-1,3-benzodioxole (1.9 g, crude) as a white solid.

### Step 2. 7-methoxy-1,3-benzodioxol-5-amine

4-Methoxy-6-nitro-1,3-benzodioxole (1.7 g, 8.62 mmol) and Pd/C (200 mg) was dissolved in EtOAc (40 mL), and then the mixture was stirred at 25° C. for 12 h under H₂ (17.38 mg, 8.62 mmol) 15 psi. LCMS showed reactant was consumed completely and desired MS was detected. The reaction was filtered and the filtrated was concentrated in vacuo. 7-methoxy-1,3-benzodioxol-5-amine (1.4 g, 8.38 mmol, 97.12% yield) was obtained as a yellow oil.

### Step 3. 6-amino-1,3-benzodioxol-4-ol

A solution of 7-methoxy-1,3-benzodioxol-5-amine (1.3 g, 7.78 mmol) in HBr/AcOH (30 mL) was stirred at 90° C. for 1 h. LCMS showed the reaction was complete mostly and the desired ms was detected. The reaction was concentrated under reduced pressure to give a crude product. The residue was purified by prep-HPLC (TFA condition:Phenomenex luna C¹⁸ 250*50 mm*10 um; mobile phase:water(TFA)-ACN]; B %: 1%-20%, 10 min) to give 6-amino-1,3-benzodioxol-4-ol (270 mg, crude) as a white solid.

### Step 4. 6-[[4-methyl-6-(methylamino)pyrimidin-2-yl]amino-],3-benzodioxol-4-ol

To a solution of 6-amino-1,3-benzodioxol-4-ol (20 mg, 130.60 μmol) and 2-chloro-N,6-dimethyl-pyrimidin-4-amine (20.58 mg, 130.60 μmol) in i-PrOH (1 mL) was added TFA (1.49 mg, 13.06 μmol, 1.01 μL), then the mixture was stirred at 120° C. for 1 h. LCMS showed starting material was consumed completely and mass of the desired compound. The reaction was filtered and concentrated under reduced pressure to give 6-[[4-methyl-6-(methylamino)pyrimidin-2-yl]amino]-1,3-benzodioxol-4-ol (35 mg, crude) as a black solid.

### Step 5. [6-[[4-methyl-6-(methylamino)pyrimidin-2-yl]amino]3-benzodioxol-4-yl]trifluoromethanesulfonate

To a solution of 6-[[4-methyl-6-(methylamino)pyrimidin-2-yl]amino]-1,3-benzodioxol-4-ol (35 mg, 127.61 μmol) and 1,1,1-trifluoro-N-phenyl-N-(trifluoromethylsulfonyl)methanesulfonamide (54.71 mg, 153.13 μmol) in MeCN (2 mL) was added K₂CO₃ (88.18 mg, 638.05 μmol), then the mixture was stirred at 20° C. for 12 h. LCMS showed starting material was consumed completely and mass of the desired compound. TLC (petroleum ether:ethyl acetate=0:1, Rf=0.6) indicated Reactant 1 was consumed completely, and one major new spot was detected. The reaction was filtered and concentrated under reduced pressure to give a residue. The residue was purified by prep-TLC (SiO₂, petroleum ether:ethyl acetate=0:1) to give [6-[[4-methyl-6-(methylamino)pyrimidin-2-yl]amino]-1,3-benzodioxol-4-yl]trifluoromethanesulfonate (25 mg, 61.53 μmol, 48.21% yield) as a white solid.

### Step 6. tert-butyl 5-[6-[[4-methyl-6-(methylamino)pyrimidin-2-yl]amino]-1,3-benzodioxol-4-yl]-2,3,4,7-tetrahydroazepine-1-carboxylate

To a solution of [6-[[4-methyl-6-(methylamino)pyrimidin-2-yl]amino]-1,3-benzodioxol-4-yl]trifluoromethanesulfonate (25 mg, 61.53 μmol), tert-butyl 5-(4,4,5,5-tetramethyl-1,3,2-dioxaborolan-2-yl)-2,3,4,7-tetrahydroazepine-1-carboxylate (39.77 mg, 123.05 μmol), K₂CO₃ (17.01 mg, 123.05 μmol) and H₂O (0.1 mL) in dioxane (1 mL) was added cyclopentyl(diphenyl)phosphane;dichloropalladium;iron (4.50 mg, 6.15 μmol), then the mixture was stirred at 100° C. for 12 h under an atmosphere of nitrogen. TLC (petroleum ether:ethyl acetate=0:1, Rf=0.3) indicated Reactant 1 was consumed completely, and one major new spot was detected. LCMS showed starting material was consumed completely and mass of the desired compound. The reaction was filtered and concentrated under reduced pressure to give a residue. The residue was purified by prep-TLC (SiO₂, petroleum ether:ethyl acetate=0:1) to give tert-butyl 5-[6-[[4-methyl-6-(methylamino)pyrimidin-2-yl]amino]-1,3-benzodioxol-4-yl]-2,3,4,7-tetrahydroazepine-1-carboxylate (20 mg, crude) as a colorless oil.

### Step 7. N4,6-dimethyl-N2-[7-(2,3,4,7-tetrahydro-1H-azepin-5-yl)-1,3-benzodioxol-5-yl]pyrimidine-2,4-diamine

To a solution of tert-butyl 5-[6-[[4-methyl-6-(methylamino)pyrimidin-2-yl]amino]-1,3-benzodioxol-4-yl]-2,3,4,7-tetrahydroazepine-1-carboxylate (20 mg, 44.10 μmol) in DCM (0.5 mL) was added TFA (0.2 mL), then the mixture was stirred at 20° C. for 30 min. LCMS showed starting material was consumed completely and mass of the desired compound. The reaction was filtered and concentrated under reduced pressure to give a residue. The residue was purified by prep-HPLC (TFA condition, column: Phenomenex luna C¹⁸ 100*40 mm*5 um; mobile phase: [water(0.1% TFA)-ACN]; B %: 1%-45%, 8 min) to give N4,6-dimethyl-N2-[7-(2,3,4,7-tetrahydro-1H-azepin-5-yl)-1,3-benzodioxol-5-yl]pyrimidine-2,4-diamine (8 mg, 17.15 μmol, 38.89% yield, TFA) (purity: 100%) as a white solid. It was rechecked by LCMS and HNMR.

¹H NMR (400 MHz, METHANOL-d₄) δ ppm 2.05 (m, 2H) 2.29 (s, 3H) 2.89 (m, 2H) 2.98 (s, 3H) 3.48 (m, 2H) 3.93 (br d, J=6.13 Hz, 2H) 5.97 (s, 1H) 6.02 (s, 2H) 6.22 (br t, J=5.94 Hz, 1H) 6.97 (br s, 1H) 7.25 (s, 1H).

### Step 8. N4,6-dimethyl-N2-[7-(1-methyl-2,3,4,7-tetrahydroazepin-5-yl)-1,3-benzodioxol-5-yl]pyrimidine-2,4-diamine

To a solution of N4,6-dimethyl-N2-[7-(2,3,4,7-tetrahydro-1H-azepin-5-yl)-1,3-benzodioxol-5-yl]pyrimidine-2,4-diamine (60 mg, 169.77 μmol) in MeOH (2 mL) was added DIEA adjust to pH=7-8, then the mixture was added (HCHO)n (50.98 mg, 1.70 mmol), then the mixture was stirred at 20° C. for 15 min. Then the mixture was added NaBH₃CN (21.34 mg, 339.54 μmol) and stirred at 20° C. 12 h. LCMS showed starting material was consumed completely and mass of the desired compound. The reaction was filtered and concentrated under reduced pressure to give a residue. The residue was purified by prep-HPLC (TFA condition, column: Phenomenex luna C¹⁸ 100*40 mm*5 um; mobile phase: [water(0.1% TFA)-ACN]; B %: 5%-50%, 8 min) to give N4,6-dimethyl-N2-[7-(1-methyl-2,3,4,7-tetrahydroazepin-5-yl)-1,3-benzodioxol-5-yl]pyrimidine-2,4-diamine (49.2 mg, 102.40 μmol, 60.32% yield, TFA) (purity: 97.726%) as a white solid.

¹H NMR (400 MHz, METHANOL-d₄) δ ppm 2.09 (m, 2H) 2.29 (s, 3H) 2.89 (m, 2H) 2.93 (s, 3H) 2.98 (s, 3H) 3.47 (m, 1H) 3.67 (br d, J=1.25 Hz, 1H) 4.05 (m, 2H) 5.96 (s, 1H) 6.02 (s, 2H) 6.18 (br t, J=6.69 Hz, 1H) 6.99 (d, J=1.75 Hz, 1H) 7.26 (d, J=1.38 Hz, 1H).

### Step 9. N2-[7-[1-(2-methoxyethyl)-2,3,4,7-tetrahydroazepin-5-yl]-1,3-benzodioxol-5-yl]-N4,6-dimethyl-pyrimidine-2,4-diamine

To a solution of N4,6-dimethyl-N2-[7-(2,3,4,7-tetrahydro-1H-azepin-5-yl)-1,3-benzodioxol-5-yl]pyrimidine-2,4-diamine (50 mg, 141.48 μmol) and 1-bromo-2-methoxy-ethane (58.99 mg, 424.43 μmol, 39.91 μL) in acetone (2 mL) was added K₂CO₃ (39.11 mg, 282.95 μmol), the mixture was stirred at 50° C. for 12 h. LCMS showed starting material was consumed completely and mass of the desired compound. The reaction was filtered and concentrated under reduced pressure to give a residue. The residue was purified by prep-HPLC (TFA condition, column: Phenomenex luna C¹⁸ 100*40 mm*5 um; mobile phase: [water(0.1% TFA)-ACN]; B %: 5%-50%, 8 min) to give N2-[7-[1-(2-methoxyethyl)-2,3,4,7-tetrahydroazepin-5-yl]-1,3-benzodioxol-5-yl]-N4,6-dimethyl-pyrimidine-2,4-diamine (28.8 mg, 54.91 μmol, 38.81% yield, TFA) (purity: 98.190%) as a white solid.

¹H NMR (400 MHz, METHANOL-d₄) δ ppm 2.08 (m, 2H) 2.29 (s, 3H) 2.92 (br t, J=5.57 Hz, 2H) 2.98 (s, 3H) 3.43 (s, 5H) 3.61 (m, 2H) 3.72 (t, J=5.00 Hz, 2H) 4.11 (m, 2H) 5.97 (s, 1H) 6.03 (s, 2H) 6.16 (br t, J=6.82 Hz, 1H) 6.98 (d, J=1.88 Hz, 1H) 7.26 (s, 1H).

### EXAMPLE 28. Synthesis of Compounds 138 and 139

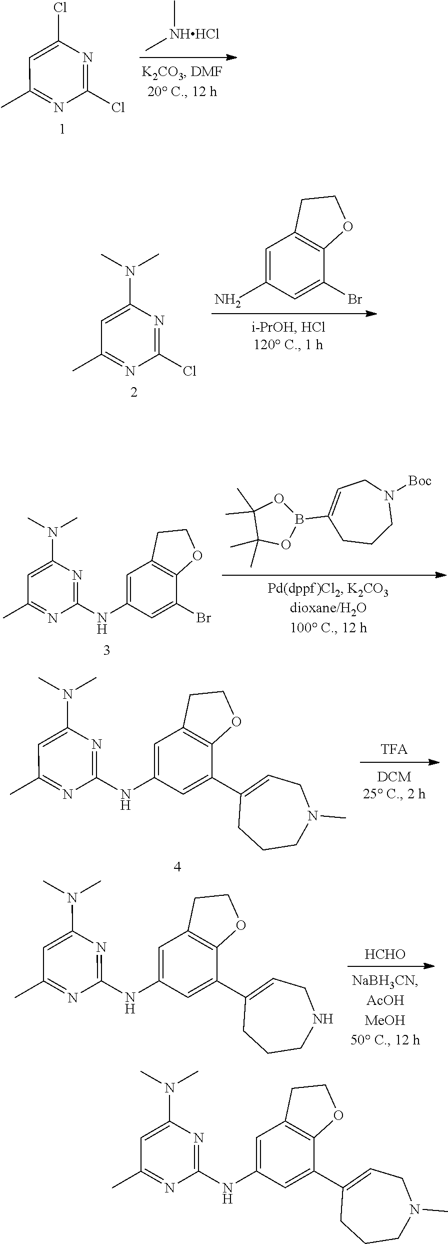

### Step 1.2-chloro-N,N,6-trimethyl-pyrimidin-4-amine

To a solution of 2,4-dichloro-6-methyl-pyrimidine (2 g, 12.27 mmol) in DMF (20 mL) was added N-methylmethanamine hydrochloride (1.00 g, 12.27 mmol) and K₂CO₃ (5.09 g, 36.81 mmol), then the mixture was stirred at 20° C. for 12 hrs. LCMS showed the reaction was complete and the desired ms was detected. The reaction mixture was added to water (50 mL), extracted with EtOAc (20 mL*5). The organic layer was dried over Na₂SO₄, concentrated to give the crude product. The residue was purified by column chromatography (SiO₂, Petroleum ether/Ethyl acetate=1/0 to 0/1) to give 2-chloro-N,N,6-trimethyl-pyrimidin-4-amine (1.4 g, crude) as white solid.

### Step 2. N2-(7-bromo-2,3-dihydrobenzofuran-5-yl)-N4,N4,6-trimethyl-pyrimidine-2,4-diamine

To the mixture of 2-chloro-N,N,6-trimethyl-pyrimidin-4-amine (80.18 mg, 467.16 μmol) and 7-bromo-2,3-dihydrobenzofuran-5-amine (100 mg, 467.16 μmol) in i-PrOH (11 mL) was added HCl (12 M, 0.1 mL), then the mixture was stirred at 120° C. in the microwave for 1 hrs. LCMS showed the starting material remained and the desired ms was detected. The reaction was concentrated under reduced pressure to give N2-(7-bromo-2,3-dihydrobenzofuran-5-yl)-N4,N4,6-trimethyl-pyrimidine-2,4-diamine (300 mg, crude) as purple solid.

### Step 3. tert-butyl 5-[5-[[4-(dimethylamino)-6-methyl-pyrimidin-2-yl]amino]-2,3-dihydrobenzofuran-7-yl]-2,3,4,7-tetrahydroazepine-1-carboxylate

To a mixture of N2-(7-bromo-2,3-dihydrobenzofuran-5-yl)-N4,N4,6-trimethyl-pyrimidine-2,4-diamine (150 mg, 429.52 μmol) and tert-butyl 5-(4,4,5,5-tetramethyl-1,3,2-dioxaborolan-2-yl)-2,3,4,7-tetrahydroazepine-1-carboxylate (138.84 mg, 429.52 μmol) in dioxane (3 mL) and H₂O (0.3 mL) was added tripotassium carbonate (118.72 mg, 859.05 μmol) and cyclopentyl(diphenyl)phosphane;dichloropalladium;iron (31.43 mg, 42.95 μmol), then the mixture was stirred at 100° C. for 12 hrs under N2. LCMS showed the reaction was complete and the desired ms was detected. The reaction was filtered and concentrated under reduced pressure to give a residue. The residue was purified by prep-TLC (SiO₂, methylene chloride/Methanol=1:1) to give tert-butyl 5-[5-[[4-(dimethylamino)-6-methyl-pyrimidin-2-yl]amino]-2,3-dihydrobenzofuran-7-yl]-2,3,4,7-tetrahydroazepine-1-carboxylate (150 mg, crude) as brown solid.

### Step 4. N4,N4,6-trimethyl-N2-[7-(2,3,4,7-tetrahydro-1H-azepin-5-yl]-2,3-dihydrobenzofuran-5-yl]pyrimidine-2,4-diamine

To a solution of tert-butyl 5-[5-[[4-(dimethylamino)-6-methyl-pyrimidin-2-yl]amino]-2,3-dihydrobenzofuran-7-yl]-2,3,4,7-tetrahydroazepine-1-carboxylate (20 mg, 42.96 μmol) in DCM (4 mL) was added TFA (1 mL), then the mixture was stirred at 25° C. for 2 hrs. LCMS showed the reaction was complete and the desired ms was detected. The reaction was filtered and concentrated under reduced pressure to give a residue. The crude product was purified by prep-HPLC(TFA condition:column:Phenomenex luna C18 100*40 mm*5 um; mobile phase:water(TFA)-ACN; B %: 1%-45%,8 min) to give N4,N4,6-trimethyl-N2-[7-(2,3,4,7-tetrahydro-1H-azepin-5-yl)-2,3-dihydrobenzofuran-5-yl]pyrimidine-2,4-diamine (17.3 mg, 36.16 μmol, 84.17% yield, TFA) as white solid.

¹H NMR (400 MHz, METHANOL-d₄) 6=7.40-7.37 (m, 1H), 7.17-7.13 (m, 1H), 6.31-6.27 (m, 1H), 6.12-6.06 (m, 1H), 4.64-4.58 (m, 2H), 3.93-3.85 (m, 2H), 3.50-3.43 (m, 2H), 3.26 (s, 8H), 2.88-2.80 (m, 2H), 2.36-2.35 (m, 3H), 2.08-2.01 (m, 2H). MS (ESI): m/z=366.1 [M+H]

### Step 5. N4,N4,6-trimethyl-N2-[7-(1-methyl-2,3,4,7-tetrahydroazepin-5-yl]-2,3-dihydrobenzo-furan-5-yl]pyrimidine-2,4-diamine

To the mixture of N4,N4,6-trimethyl-N2-[7-(2,3,4,7-tetrahydro-1H-azepin-5-yl)-2,3-dihydrobenzofuran-5-yl]pyrimidine-2,4-diamine (100 mg, 273.62 mol) and formaldehyde (16.43 mg, 547.24 μmol, 15.17 μL) in MeOH (2 mL) was added sodium cyanoboranuide (85.97 mg, 1.37 mmol) and CH₃COOH (0.2 mL), then the mixture was stirred at 50° C. for 12 hrs. LCMS showed the reaction was complete and the desired ms was detected. The reaction was filtered and concentrated under reduced pressure to give a residue. The crude product was purified by prep-HPLC (TFA condition:column:Phenomenex luna C18 100*40 mm*5 um; mobile phase:water(TFA)-ACN; B %: 1%-45%,8 min) to give N4,N4,6-trimethyl-N2-[7-(1-methyl-2,3,4,7-tetrahydroazepin-5-yl)-2,3-dihydrobenzofuran-5-yl]pyrimidine-2,4-diamine (24.3 mg, 49.34 μmol, 18.03% yield, TFA) as pale yellow solid.

¹H NMR (400 MHz, METHANOL-d₄) δ=7.34 (s, 1H), 7.19-7.13 (m, 1H), 6.33-6.26 (m, 1H), 6.09-6.01 (m, 1H), 4.67-4.56 (m, 2H), 4.17-3.89 (m, 2H), 3.67 (br s, 1H), 3.28-3.24 (m, 2H), 3.24-3.18 (m, 6H), 2.97 (s, 3H), 2.90-2.78 (m, 2H), 2.37-2.26 (m, 3H), 2.19-1.96 (m, 2H). MS (ESI): m/z=380.1 [M+H].

### EXAMPLE 29. Synthesis of Compound 141

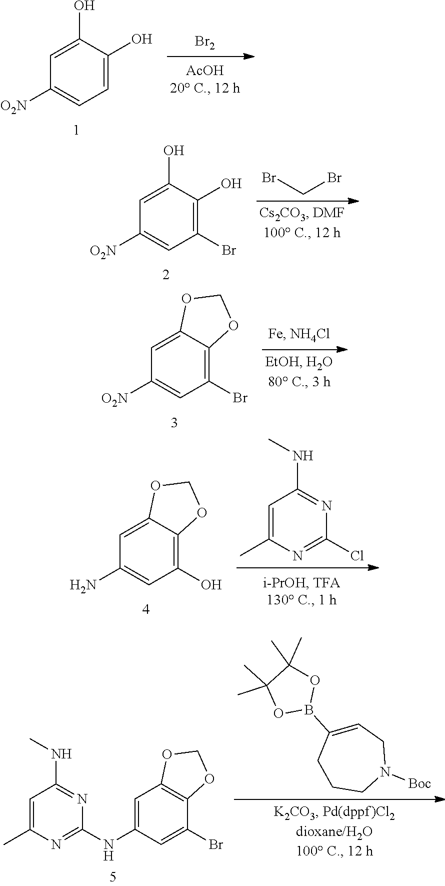

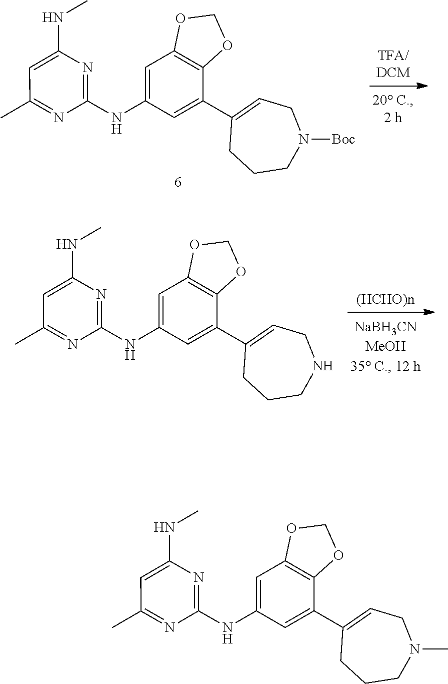

### Step 1. 3-bromo-5-nitro-benzene-1,2-diol

4-Nitrobenzene-1,2-diol (28 g, 180.52 mmol) was dissolved in AcOH (300 mL), and then to the mixture was added Br₂ (28.85 g, 180.52 mmol, 8.25 mL), and then the mixture was stirred at 20° C. for 12 h. LCMS showed the reaction was complete mostly and the desired ms was detected. The reaction mixture was added to water (500 mL), extracted with EtOAc (1000 mL*3). The combined organic layers were washed with 500 mL of brine, dried over Na₂SO₄, filtered and concentrated under reduced pressure to give a residue. The crude product was purified by column chromatography on silica gel (Petroleum ether:Ethyl acetate=1:0-2:1) to give 3-bromo-5-nitro-benzene-1,2-diol (21 g, crude) as a yellow solid.

### Step 2. 4-bromo-6-nitro-1,3-benzodioxole

To a solution of 3-bromo-5-nitro-benzene-1,2-diol (5 g, 21.37 mmol) and dibromomethane (7.43 g, 42.73 mmol) in DMF (50 mL) was added C_{S2}CO₃ (20.89 g, 64.10 mmol), then the mixture was stirred at 100° C. for 12 h under sealed tube. TLC (petroleum ether:ethyl acetate=3:1, Rf=0.8) indicated Reactant 1 remained, and two major new spots were detected. LCMS showed starting material remained. Three reactions were combined. The reaction was added into water (1000 mL), then extracted with ethyl acetate (1000*3 mL). The combined organic layers were washed with brine (1500*3 mL) and dried over anhydrous sodium sulfate, filtered and concentrated under reduced pressure to give a residue. The residue was purified by flash chromatography (silica gel, petroleum ether/ethyl acetate=1/0 to 100/1) to give 4-bromo-6-nitro-1,3-benzodioxole (11.96 g, crude) as a yellow solid. ¹HNMR indicated desired compound.

### Step 3. 7-bromo-1,3-benzodioxol-5-amine

To a solution of 4-bromo-6-nitro-1,3-benzodioxole (11.96 g, 48.61 mmol), NH₄Cl (26.00 g, 486.15 mmol) and H₂O (15 mL) in EtOH (150 mL) was added Fe (13.57 g, 243.07 mmol), then the mixture was stirred at 80° C. for 3 h. TLC (petroleum ether:ethyl acetate=3:1, Rf=0.4) indicated Reactant 1 was consumed completely, and two major new spots were detected. LCMS showed mass of the desired compound. The reaction was filtered and concentrated under reduced pressure, then added water (250 mL), then extracted with ethyl acetate (3×300 mL) and dried over anhydrous sodium sulfate, filtered and concentrated under reduced pressure to give a residue. The residue was purified by reversed-phase HPLC (0.1% TFA condition) to give 7-bromo-1,3-benzodioxol-5-amine (9.5 g, 43.98 mmol, 90.46% yield) as a brown solid.

### Step 4. N2-(7-bromo-1,3-benzodioxol-5-yl)-N4,6-dimethyl-pyrimidine-2,4-diamine

To a solution of 7-bromo-1,3-benzodioxol-5-amine (5 g, 23.14 mmol) and 2-chloro-N,6-dimethyl-pyrimidin-4-amine (3.65 g, 23.14 mmol) in i-PrOH (50 mL) was added TFA (263.90 mg, 2.31 mmol, 178.31 μL), then the mixture was stirred at 130° C. for 1 h under sealed tube. TLC (ethyl acetate:methanol=10:1, Rf=0.4) indicated Reactant 1 was consumed completely, and one major new spots were detected. LCMS showed starting material was consumed completely and mass of the desired compound. The reaction was filtered and concentrated under reduced pressure to give N2-(7-bromo-1,3-benzodioxol-5-yl)-N4,6-dimethyl-pyrimidine-2,4-diamine (5.9 g, crude) as a brown solid. ¹HNMR indicated desired compound.

¹H NMR (400 MHz, METHANOL-d₄) δ ppm 2.29 (s, 3H) 2.98 (s, 3H) 6.01 (s, 1H) 6.08 (s, 2H) 7.07 (d, J=1.50 Hz, 1H) 7.22 (d, J=1.75 Hz, 1H)

### Step 5. tert-butyl5-[6-[[4-methyl-6-(methylamino)pyrimidin-2-yl]amino]-1,3-benzodioxol-4-yl-2,3,4,7-tetrahydroazepine-1-carboxylate

To a solution of N2-(7-bromo-1,3-benzodioxol-5-yl)-N4,6-dimethyl-pyrimidine-2,4-diamine (3 g, 8.90 mmol) and tert-butyl 5-(4,4,5,5-tetramethyl-1,3,2-dioxaborolan-2-yl)-2,3,4,7-tetrahydroazepine-1-carboxylate (2.88 g, 8.90 mmol) in dioxane (60 mL) and H₂O (6 mL) was added K₂CO₃ (2.46 g, 17.80 mmol) and cyclopentyl(diphenyl)phosphane;dichloropalladium;iron (651.04 mg, 889.76 μmol) under an atmosphere of nitrogen, then the mixture was stirred at 100° C. for 12 h under an atmosphere of nitrogen. TLC (ethyl acetate:methanol=10:1, Rf=0.2) indicated Reactant 1 was consumed completely, and two major new spots were detected. LCMS showed starting material was consumed completely and mass of the desired compound. The reaction was filtered and concentrated under reduced pressure to give a residue. The residue was purified by flash chromatography (silica gel, petroleum ether/ethyl acetate=1/0 to 1/2) to give tert-butyl 5-[6-[[4-methyl-6-(methylamino)pyrimidin-2-yl]amino]-1,3-benzodioxol-4-yl]-2,3,4,7-tetrahydroazepine-1-carboxylate (3.44 g, 7.58 mmol, 85.25% yield) as a brown solid.

¹H NMR (400 MHz, METHANOL-d₄) δ ppm 1.46 (br d, J=5.13 Hz, 9H) 1.88 (quin, J=5.82 Hz, 2H) 2.16 (s, 3H) 2.67 (m, 2H) 2.88 (s, 3H) 3.35 (s, 1H) 3.61 (br t, J=5.94 Hz, 2H) 4.02 (m, 2H) 5.78 (s, 1H) 5.89 (s, 2H) 6.17 (m, 1H) 6.95 (br d, J=15.51 Hz, 1H) 7.29 (d, J=1.88 Hz, 1H)

### Step 6. N4,6-dimethyl-N2-[7-(2,3,4,7-tetrahydro-1H-azepin-5-yl)-1,3-benzodioxol-5-yl]pyrimidine-2,4-diamine

To a solution of tert-butyl 5-[6-[[4-methyl-6-(methylamino)pyrimidin-2-yl]amino]-1,3-benzodioxol-4-yl]-2,3,4,7-tetrahydroazepine-1-carboxylate (500 mg, 1.10 mmol) in DCM (5 mL) was added TFA (4 mL), then the mixture was stirred at 20° C. for 30 min. LCMS showed starting material was consumed completely and mass of the desired compound. The reaction was filtered and concentrated under reduced pressure to give a residue. The residue was purified by prep-HPLC (basic condition, column: Waters Xbridge Prep OBD C¹⁸ 150*40 mm*10 um; mobile phase: [water(0.05% NH₃H₂O+10 mM NH₄HCO₃)-ACN]; B %: 5%-45%, 8 min) to give N4,6-dimethyl-N2-[7-(2,3,4,7-tetrahydro-1H-azepin-5-yl)-1,3-benzodioxol-5-yl]pyrimidine-2,4-diamine (270.5 mg, 765.38 μmol, 69.43% yield) (purity: 99.502%) as a white solid.

### Step 7. N4,6-dimethyl-N2-[7-(1-methyl-2,3,4,7-tetrahydroazepin-5-yl)-1,3-benzodioxol-5-yl]pyrimidine-2,4-diamine

To a solution of N4,6-dimethyl-N2-[7-(2,3,4,7-tetrahydro-1H-azepin-5-yl)-1,3-benzodioxol-5-yl]pyrimidine-2,4-diamine (1.5 g, 4.24 mmol) in MeOH (50 mL) was added DIEA adjust to pH=7-8, then the mixture was added (HCHO)n (382.31 mg, 12.73 mmol), then the mixture was stirred at 20° C. for 15 min. Then the mixture was added sodium cyanoboranuide (533.44 mg, 8.49 mmol) and stirred at 35° C. 12 h. LCMS showed starting material was consumed completely and mass of the desired compound. The reaction was filtered and concentrated under reduced pressure to give a residue. The residue was purified by prep-HPLC (basic condition, column: Waters Xbridge Prep OBD C¹⁸ 150*40 mm*10 um; mobile phase: [water(0.05% NH₃H₂O+10 mM NH₄HCO₃)-ACN]; B %: 5%-50%, 8 min) to give N4,6-dimethyl-N2-[7-(1-methyl-2,3,4,7-tetrahydroazepin-5-yl)-1,3-benzodioxol-5-yl]pyrimidine-2,4-diamine (940.9 mg, 2.56 mmol, 60.33% yield) (purity: 98.466%) as a brown solid. It was rechecked by LCMS and HNMR.

¹H NMR (400 MHz, METHANOL-d₄) δ ppm 1.83 (quin, J=5.50 Hz, 2H) 2.16 (s, 3H) 2.36 (s, 3H) 2.71 (m, 2H) 2.89 (m, 5H) 3.28 (br d, J=6.38 Hz, 2H) 5.78 (s, 1H) 5.89 (s, 2H) 6.15 (t, J=6.44 Hz, 1H) 6.98 (br s, 1H) 7.30 (d, J=2.00 Hz, 1H).

### EXAMPLE 30. Synthesis of Compound 142

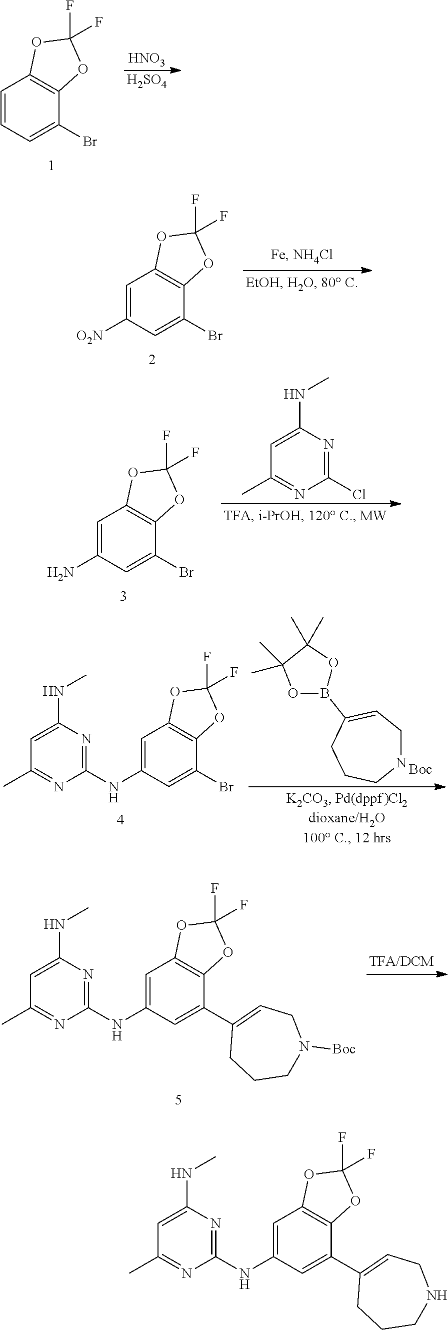

### Step 1. 4-bromo-2,2-difluoro-6-nitro-],3-benzodioxole

A mixture of HNO₃ (0.5 mL)/H₂SO₄ (0.5 mL) was added drop wise to the mixture of 4-bromo-2,2-difluoro-1,3-benzodioxole (500 mg, 2.11 mmol) in H₂SO₄ (1 mL) at 0° C., then the reaction mixture was stirred at 0° C. for 1 hr. TLC (petroleum ether:ethyl acetate=5:1, Rf=0.75) showed the reaction was complete and 2 new spots were formed. The reaction mixture was added dropwise the ice-water (20 mL), extracted with EtOAc (10 mL×2). The organic layer was dried over Na₂SO₄, concentrated to give a residue. The reside was purified by column chromatography on silica gel (petroleum ether:ethyl acetate=0: 1-100:1) to give 4-bromo-2,2-difluoro-6-nitro-1,3-benzodioxole (100 mg, 354.62 μmol, 16.81% yield) as yellow oil.

### Step 2. 7-bromo-2,2-difluoro-1,3-benzodioxol-5-amine

To a mixture of 4-bromo-2,2-difluoro-6-nitro-1,3-benzodioxole (100 mg, 354.62 mol.) in EtOH (3 mL) and H₂O (0.6 mL) was added Fe (99.02 mg, 1.77 mmol.) and ammonia hydrochloride (189.69 mg, 3.55 mmol.), then the mixture was stirred at 80° C. for 1 h. LCMS showed the reaction was complete mostly and the desired ms was detected. The reaction was filtered and concentrated under reduced pressure to give 7-bromo-2,2-difluoro-1,3-benzodioxol-5-amine (36 mg, crude) as a light yellow solid.

### Step 3. N2-(7-bromo-2,2-difluoro-1,3-benzodioxol-5-yl)-N4,6-dimethyl-pyrimidine-2,4-diamine

To a mixture of 7-bromo-2,2-difluoro-1,3-benzodioxol-5-amine (36 mg, 142.85 mol) and 2-chloro-N,6-dimethyl-pyrimidin-4-amine (22.51 mg, 142.85 μmol) in i-PrOH (3 mL) was added HCl (12 M, 11.90 μL), then the mixture was stirred at 130° C. in the microwave for 1 hrs. LCMS showed the reaction was complete mostly and the desired ms was detected. The reaction was concentrated under reduced pressure to give N2-(7-bromo-2,2-difluoro-1,3-benzodioxol-5-yl)-N4,6-dimethyl-pyrimidine-2,4-diamine (60 mg, crude) as a light yellow solid.

### Step 4. 5-[2,2-difluoro-6-[[4-methyl-6-(methylamino)pyrimidin-2-yl]amino]-1,3-benzodioxol-4-yl]-2,3,4,7-tetrahydroazepine-1-carboxylate

To a mixture of N2-(7-bromo-2,2-difluoro-1,3-benzodioxol-5-yl)-N4,6-dimethyl-pyrimidine-2,4-diamine (20 mg, 53.60 μmol.) and tert-butyl 5-(4,4,5,5-tetramethyl-1,3,2-dioxaborolan-2-yl)-2,3,4,7-tetrahydroazepine-1-carboxylate (17.32 mg, 53.60 μmol) in dioxane (2 mL) and H₂O (0.2 mL) was added tripotassium carbonate (14.81 mg, 107.19 mol, 6.47 μL) and cyclopentyl(diphenyl)phosphane;dichloropalladium;iron (3.92 mg, 5.36 μmol), then the mixture was stirred at 100° C. for 12 hrs under N2. LCMS showed the reaction was complete and the desired ms was detected. The reaction was filtered and concentrated under reduced pressure to give tert-butyl 5-[2,2-difluoro-6-[[4-methyl-6-(methylamino)pyrimidin-2-yl]amino]-1,3-benzodioxol-4-yl]-2,3,4,7-tetrahydroazepine-1-carboxylate (40 mg, crude) as a black solid.

### Step 5. N2-[2,2-difluoro-7-(2,3,4,7-tetrahydro-1H-azepin-5-yl)-1,3-benzodioxol-5-yl]-N4,6-dimethyl-pyrimidine-2,4-diamine

To a mixture of tert-butyl 5-[2,2-difluoro-6-[[4-methyl-6-(methylamino)pyrimidin-2-yl]amino]-1,3-benzodioxol-4-yl]-2,3,4,7-tetrahydroazepine-1-carboxylate (40 mg, 81.71 μmol) in DCM (2 mL) was added TFA (0.4 mL). Then the mixture was stirred at 25° C. for 2 hrs. LCMS showed the reaction was complete mostly and the desired ms was detected. The reaction was concentrated under reduced pressure to give a residue. The crude product was purified by prep-HPLC(TFA condition:column: Phenomenex luna C18 100*40 mm*5 um; mobile phase:water(TFA)-ACN]; B %: 5%-50%, 8 min) to give N2-[2,2-difluoro-7-(2,3,4,7-tetrahydro-1H-azepin-5-yl)-1,3-benzodioxol-5-yl]-N4,6-dimethyl-pyrimidine-2,4-diamine (11.3 mg, 22.49 μmol, 27.52% yield, TFA) as a white solid.

¹H NMR (400 MHz, METHANOL-d₄) δ ppm 7.74 (br s, 1H), 7.26 (s, 1H), 6.28 (t, J=6.3 Hz, 1H), 6.03 (s, 1H), 3.98 (d, J=6.4 Hz, 2H), 3.52-3.48 (m, 2H), 3.00 (s, 3H), 2.94-2.88 (m, 2H), 2.32 (s, 3H), 2.14-2.06 (m, 2H). MS (ESI): m/z=390.1 [M+H]⁺

### EXAMPLE 31. Synthesis of Compound 143

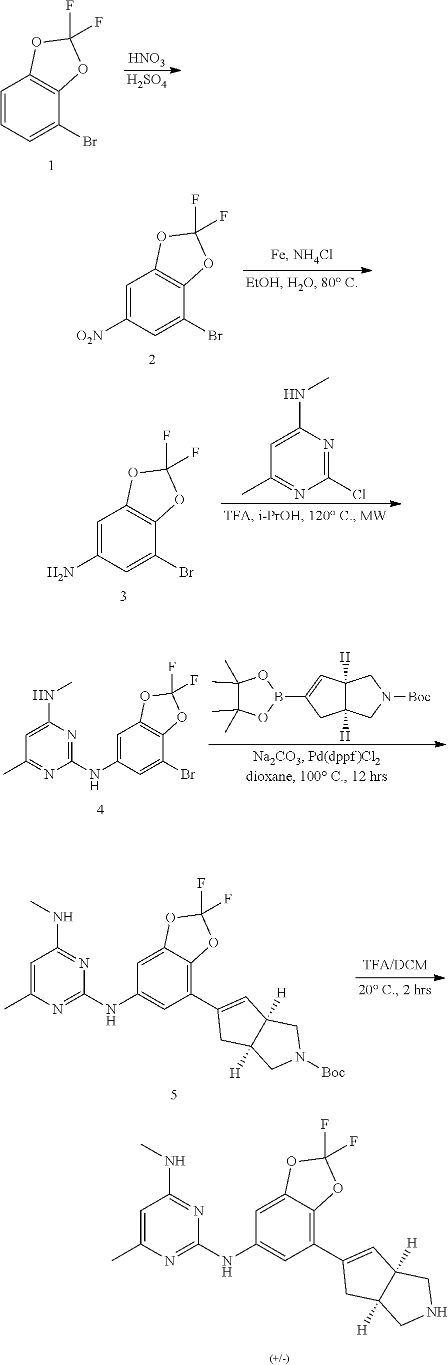

### Step 1. 4-bromo-2,2-difluoro-6-nitro-1,3-benzodioxole

A mixture of HNO₃ (0.5 mL)/H₂SO₄ (0.5 mL) was added drop wise to the mixture of 4-bromo-2,2-difluoro-1,3-benzodioxole (500 mg, 2.11 mmol, 1 eq.) in H₂SO₄ (1 mL) at 0° C., then the reaction mixture was stirred at 0° C. for 1 hr. TLC (petroleum ether:ethyl acetate=5:1, Rf=0.75) showed the reaction was complete and 2 new spots were formed. The reaction mixture was added dropwise the ice-water (20 mL), extracted with EtOAc (10 mL*2). The organic layer was dried over Na₂SO₄, concentrated to give a residue. The reside was purified by column chromatography on silica gel (petroleum ether:ethyl acetate=0:1-100; 1) to give 4-bromo-2,2-difluoro-6-nitro-1,3-benzodioxole (100 mg, 354.62 μmol, 16.81% yield) as yellow oil.

### Step 2. 7-bromo-2,2-difluoro-1,3-benzodioxol-5-amine

To a mixture of 4-bromo-2,2-difluoro-6-nitro-1,3-benzodioxole (100 mg, 354.62 mol, 1 eq.) in EtOH (3 mL) and H₂O (0.6 mL) was added Fe (99.02 mg, 1.77 mmol, 5 eq.) and ammonia hydrochloride (189.69 mg, 3.55 mmol, 10 eq.), then the mixture was stirred at 80° C. for 1 h. LCMS showed the reaction was complete mostly and the desired ms was detected. The reaction was filtered and concentrated under reduced pressure to give 7-bromo-2,2-difluoro-1,3-benzodioxol-5-amine (36 mg, crude) as a light yellow solid.

### Step 3. N2-(7-bromo-2,2-difluoro-1,3-benzodioxol-5-yl)-N4,6-dimethyl-pyrimidine-2,4-diamine

To a mixture of 7-bromo-2,2-difluoro-1,3-benzodioxol-5-amine (36 mg, 142.85 mol, 1 eq.) and 2-chloro-N,6-dimethyl-pyrimidin-4-amine (22.51 mg, 142.85 μmol, 1 eq.) in i-PrOH (3 mL) was added HCl (12 M, 11.90 μL, 1 eq.), then the mixture was stirred at 130° C. in the microwave for 1 hrs. LCMS showed the reaction was complete mostly and the desired ms was detected. The reaction was concentrated under reduced pressure to give N2-(7-bromo-2,2-difluoro-1,3-benzodioxol-5-yl)-N4,6-dimethyl-pyrimidine-2,4-diamine (60 mg, crude) as a light yellow solid.

### Step 4. tert-butyl 5-[2,2-difluoro-6-[[4-methyl-6-(methylamino)pyrimidin-2-yl]amino]-1,3-benzodioxol-4-yl]-3,3a,6,6a-tetrahydro-1H-cyclopenta[c]pyrrole-2-carboxylate

To a mixture of N2-(7-bromo-2,2-difluoro-1,3-benzodioxol-5-yl)-N4,6-dimethyl-pyrimidine-2,4-diamine (30 mg, 80.40 μmol, 1 eq.) and tert-butyl 5-(4,4,5,5-tetramethyl-1,3,2-dioxaborolan-2-yl)-3,3a,6,6a-tetrahydro-1H-cyclopenta[c]pyrrole-2-carboxylate (26.95 mg, 80.40 μmol, 1 eq.) in dioxane (1 mL) and H₂O (0.1 mL) was added cyclopentyl(diphenyl)phosphane;dichloropalladium;iron (5.88 mg, 8.04 μmol, 0.1 eq.) and disodium carbonate (17.04 mg, 160.79 μmol, 6.73 μL, 2 eq.), then the mixture was stirred at 100° C. for 12 hrs under N2. LCMS showed the reaction was complete and the desired ms was detected. The reaction was concentrated under reduced pressure to give a residue. The residue was purified by prep-TLC (dichloromethane/methyl alcohol=10:1, Rf=0.4) to give tert-butyl 5-[2,2-difluoro-6-[[4-methyl-6-(methylamino)pyrimidin-2-yl]amino]-1,3-benzodioxol-4-yl]-3,3a,6,6a-tetrahydro-1H-cyclopenta[c]pyrrole-2-carboxylate (20 mg, crude) as a brown oil.

### Step 5. N2-[7-(1, 2, 3, 3a,6,6a-hexahydrocyclopenta[c]pyrrol-5-yl)-2,2-difluoro-1,3-benzodioxol-5-yl]-N4,6-dimethyl-pyrimidine-2,4-diamine

To a mixture of tert-butyl 5-[2,2-difluoro-6-[[4-methyl-6-(methylamino)pyrimidin-2-yl]amino]-1,3-benzodioxol-4-yl]-3,3a,6,6a-tetrahydro-1H-cyclopenta[c]pyrrole-2-carboxylate (20 mg, 39.88 μmol, 1 eq.) in DCM (1 mL) was added TFA (0.2 mL), then the mixture was stirred at 25° C. for 1 h. LCMS showed the reaction was complete and the desired ms was detected. The reaction was filtered and concentrated under reduced pressure to give a residue. The crude product was purified by prep-HPLC(TFA condition:column:Phenomenex luna C18 80*40 mm*3 um; mobile phase:water (TFA)-ACN]; B %: 15%-35%, 7 min) to give N2-[7-(1,2,3,3a,6,6a-hexahydrocyclopenta[c]pyrrol-5-yl)-2,2-difluoro-1,3-benzodioxol-5-yl]-N4,6-dimethyl-pyrimidine-2,4-diamine (3.6 mg, 8.97 μmol, 22.49% yield) as a white solid.

¹H NMR (400 MHz, METHANOL-d₄) δ=7.69 (br s, 1H), 7.39-7.12 (m, 1H), 6.36 (br s, 1H), 6.03 (s, 1H), 3.87 (br s, 1H), 3.53 (br s, 1H), 3.49-3.41 (m, 2H), 3.23-3.11 (m, 2H), 3.00 (s, 3H), 2.98-2.59 (m, 2H), 2.32 (br s, 3H). MS (ESI): m/z=402.1 [M+H]⁺

### EXAMPLE 32. Synthesis of Compounds 144 and 146

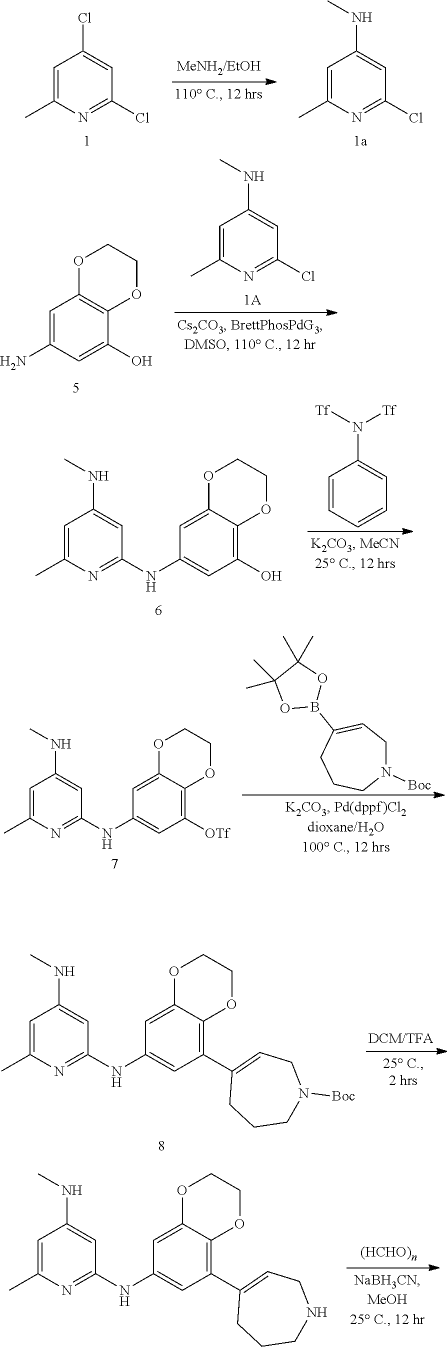

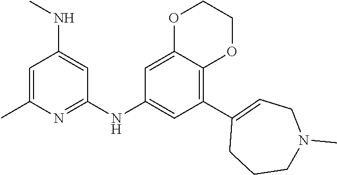

### Step 1. 2-chloro-N,6-dimethyl-pyridin-4-amine

The mixture of 2,4-dichloro-6-methyl-pyridine (5 g, 30.86 mmol) in CH₃NH₂/EtOH (60 g, 1.93 mol, 66 mL) was stirred at 110° C. for 12 h in sealed tube. TLC:Petroleum ether:Ethyl acetate=1:1 (Rf=0.4) showed the reaction was complete. The reaction mixture was filtered and concentrated under reduced pressure to give a crude product. The crude product was purified by reversed-phase HPLC (0.1% TFA condition) to give 2-chloro-N,6-dimethyl-pyridin-4-amine (3 g, crude) as a pale yellow solid.

### Step 2. 7-[[6-methyl-4-(methylamino)-2-pyridyl]amino]-2,3-dihydro-1,4-benzodioxin-5-ol

To a mixture of 7-amino-2,3-dihydro-1,4-benzodioxin-5-ol (176 mg, 1.05 mmol) in DMSO (15 mL) was added C_{S2}CO₃ (1.03 g, 3.16 mmol), 2-chloro-N,6-dimethyl-pyridin-4-amine (164.89 mg, 1.05 mmol) and BrettPhosPdG3 (190.89 mg, 210.57 μmol), then the mixture was stirred at 110° C. for 12 hrs under N₂ atmosphere. LCMS showed the reaction was complete. The reaction mixture was filtered and concentrated under reduced pressure to give a crude product. The residue was purified by prep-HPLC (TFA condition; column: Phenomenex luna C18 100*40 mm*5 um; mobile phase:water(TFA)-ACN; B %: 1%-45%,8 min). Compound 7-[[6-methyl-4-(methylamino)-2-pyridyl]amino]-2,3-dihydro-1,4-benzodioxin-5-ol (50 mg, crude) was obtained as a brown solid.

### Step 3. [7-[[6-methyl-4-(methylamino)-2-pyridyl]amino]-2,3-dihydro-1,4-benzodioxin-5-yl trifluoromethanesulfonate

To a solution of 7-[[6-methyl-4-(methylamino)-2-pyridyl]amino]-2,3-dihydro-1,4-benzodioxin-5-ol (50 mg, 174.03 μmol) in MeCN (2 mL) was added K₂CO₃ (48.10 mg, 348.05 μmol), then 1,1,1-trifluoro-N-phenyl-N(trifluoromethylsulfonyl) methanesulfonamide (93.26 mg, 261.04 mol) was added to above solution at 0° C., the mixture was stirred at 25° C. for 12 hr. LCMS showed the reaction was complete. The reaction mixture was filtered and concentrated under reduced pressure to give a crude product. The residue was purified by prep-TLC (Petroleum ether/Ethyl acetate=0:1, Rf=0.4) to give [7-[[6-methyl-4-(methylamino)-2-pyridyl]amino]-2,3-dihydro-1,4-benzodioxin-5-yl]trifluoromethanesulfonate (91 mg, crude) as a yellow solid.

### Step 4. tert-butyl 5-[7-[[6-methyl-4-(methylamino)-2-pyridyl]amino]-2,3-dihydro-1,4-benzodioxin-5-yl]-2,3,4,7-tetrahydroazepine-1-carboxylate

A mixture of [7-[[6-methyl-4-(methylamino)-2-pyridyl]amino]-2,3-dihydro-1,4-benzodioxin-5-yl]trifluoromethanesulfonate (91 mg, 216.99 μmol), tert-butyl 5-(4,4,5,5-tetramethyl-1,3,2-dioxaborolan-2-yl)-2,3,4,7-tetrahydroazepine-1-carboxylate (84.17 mg, 260.39 mol), K₂CO₃ (59.98 mg, 433.98 μmol) and Pd(dppf)Cl₂ (15.88 mg, 21.70 mol) in H₂O (0.3 mL) and dioxane (3 mL) was degassed and purged with N2 for 3 times, and then the mixture was stirred at 100° C. for 12 hr under N₂ atmosphere. LCMS showed the reaction was complete. The reaction mixture was filtered and concentrated under reduced pressure to give a crude product. The residue was purified by prep-TLC (Petroleum ether/Ethyl acetate=0:1, Rf=0.4) to give tert-butyl 5-[7-[[6-methyl-4-(methylamino)-2-pyridyl]amino]-2,3-dihydro-1,4-benzodioxin-5-yl]-2,3,4,7-tetrahydroazepine-1-carboxylate (77 mg, crude) as a yellow solid.

### Step 5. N4,6-dimethyl-N2-[5-(2,3,4,7-tetrahydro-1H-azepin-5-yl]-2,3-dihydro-1,4-benzodioxin-7-yl]pyridine-2,4-diamine

The mixture of tert-butyl 5-[7-[[6-methyl-4-(methylamino)-2-pyridyl]amino]-2,3-dihydro-1,4-benzodioxin-5-yl]-2,3,4,7-tetrahydroazepine-1-carboxylate (57 mg, 122.17 μmol) in TFA (0.5 mL) and DCM (1 mL) was stirred at 25° C. for 2 h. LCMS showed the reaction was complete. The reaction mixture was filtered and concentrated under reduced pressure to give a crude product. A part of the residue was purified by prep-HPLC (TFA condition; column: Phenomenex luna C18 100*40 mm*5 um; mobile phase:water (TFA)-ACN; B %: 1%-45%,8 min) to give N4,6-dimethyl-N2-[5-(2,3,4,7-tetrahydro-1H-azepin-5-yl)-2,3-dihydro-1,4-benzodioxin-7-yl]pyridine-2,4-diamine (18.5 mg, 38.58 μmol, 31.58% yield, TFA) as a brown gum. And the rest part residue was concentrated to give N4,6-dimethyl-N2-[5-(2,3,4,7-tetrahydro-1H-azepin-5-yl)-2,3-dihydro-1,4-benzodioxin-7-yl]pyridine-2,4-diamine (36 mg, crude) as brown oil which was used for next step without purification.

¹H NMR (400 MHz, METHANOL-d₄) δ ppm 6.85-6.73 (m, 1H), 6.70-6.57 (m, 1H), 6.23-5.99 (m, 1H), 5.89 (br t, J=6.3 Hz, 1H), 5.76-5.59 (m, 1H), 4.34-4.23 (m, 4H), 3.87 (br d, J=6.4 Hz, 2H), 3.53-3.43 (m, 2H), 3.31 (s, 2H), 2.90-2.71 (m, 5H), 2.46-2.24 (m, 3H), 2.13-1.98 (m, 2H)

### Step 6. N4,6-dimethyl-N2-[5-(1-methyl-2,3,4,7-tetrahydroazepin-5-yl]-2,3-dihydro-1,4-benzodioxin-7-yl]pyridine-2,4-diamine

To a solution of N4,6-dimethyl-N2-[5-(2,3,4,7-tetrahydro-1H-azepin-5-yl)-2,3-dihydro-1,4-benzodioxin-7-yl]pyridine-2,4-diamine (36 mg, 98.24 μmol) in MeOH (2 mL) was added DIEA adjust to pH=7-8, AcOH was added to above solution to adjust pH=5-6. (HCHO)ₙ(29.50 mg, 982.38 mol) and sodium cyanoboranuide (12.35 mg, 196.48 μmol) was added to above solution. Then the mixture was stirred at 25° C. for 12 hr. LCMS showed the reaction was complete. The reaction mixture was filtered and concentrated under reduced pressure to give a crude product. The residue was purified by prep-HPLC (TFA condition; column: Phenomenex Luna 80*30 mm*3 um; mobile phase: [water(0.1% TFA)-ACN]; B %: 15%-45%,8 min). N4,6-dimethyl-N2-[5-(1-methyl-2,3,4,7-tetrahydroazepin-5-yl)-2,3-dihydro-1,4-benzodioxin-7-yl]pyridine-2,4-diamine (29.8 mg, 60.39 μmol, 61.47% yield, TFA, purity: 100%) was obtained as a yellow gum.

¹H NMR (400 MHz, METHANOL-d₄) δ ppm 6.80 (br s, 1H), 6.66 (br s, 1H), 6.20-5.98 (m, 1H), 5.85-5.81 (m, 1H), 5.67 (br s, 1H), 4.33-4.27 (m, 4H), 4.09-3.91 (m, 2H), 3.67 (br s, 1H), 3.49-3.40 (m, 1H), 2.92 (s, 3H), 2.85-2.71 (m, 5H), 2.41-2.28 (m, 3H), 2.14-2.05 (m, 2H).

### EXAMPLE 33. Synthesis of Compound 147

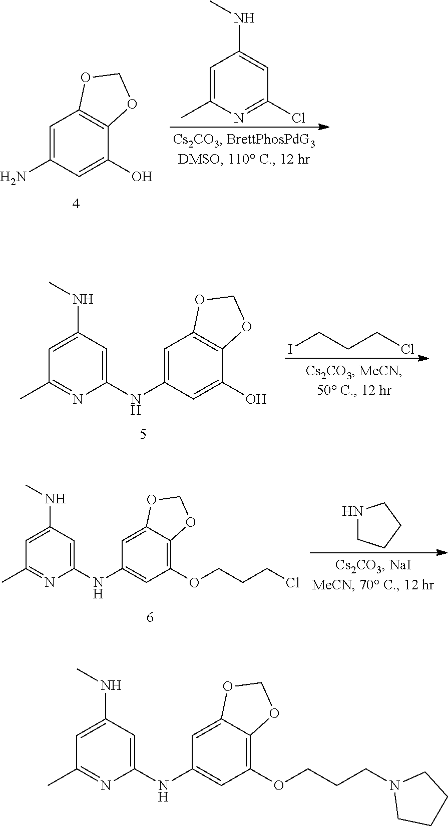

### Step 1. 6-[[6-methyl-4-(methylamino)-2-pyridyl]amino]-1,3-benzodioxol-4-ol

To a mixture of 6-amino-1,3-benzodioxol-4-ol (20 mg, 130.60 μmol) in DMSO (2 mL) was added 2-chloro-N,6-dimethyl-pyridin-4-amine (20.45 mg, 130.60 μmol), C_{S2}CO₃ (127.66 mg, 391.81 mol) and BrettPhosPdG₃ (23.68 mg, 26.12 mol), then the mixture was stirred at 110° C. for 12 hrs under N₂ atmosphere. LCMS showed the reaction was complete. The five batches of the reaction mixture were mixed together for work up. The reaction mixture was filtered and concentrated under reduced pressure to give a residue, then diluted with H₂O 30 mL and extracted with EtOAc 90 mL (30 mL*3). The combined organic layers were washed with brine 50 mL, dried over Na₂SO₄, filtered and concentrated under reduced pressure to give a residue. The residue was purified by prep-HPLC (TFA condition; column: Phenomenex luna C18 100*40 mm*5 um; mobile phase: [water(0.1% TFA)-ACN]; B %: 1%-45%,8 min). 6-[[6-methyl-4-(methylamino)-2-pyridyl]amino]-1,3-benzodioxol-4-ol (43 mg, crude) was obtained as a brown solid.

### Step 2. N2-[7-(3-chloropropoxy)-1,3-benzodioxol-5-yl]-N4,6-dimethyl-pyridine-2,4-diamine

A mixture of 6-[[6-methyl-4-(methylamino)-2-pyridyl]amino]-1,3-benzodioxol-4-ol (33 mg, 120.75 μmol) and 1-chloro-3-iodo-propane (24.69 mg, 120.75 μmol, 12.97 μL) in MeCN (2 mL) was stirred at 50° C. for 12 h. The reaction mixture was filtered and concentrated under reduced pressure to give N2-[7-(3-chloropropoxy)-1,3-benzodioxol-5-yl]-N4,6-dimethyl-pyridine-2,4-diamine (43 mg, crude) was obtained as yellow oil.

### Step 3. N4,6-dimethyl-N2-[7-(3-pyrrolidin-1-ylpropoxy)-1,3-benzodioxol-5-yl]pyridine-2,4-diamine

To a mixture of N2-[7-(3-chloropropoxy)-1,3-benzodioxol-5-yl]-N4,6-dimethyl-pyridine-2,4-diamine (43 mg, 122.92 μmol) in MeCN (1.99 mL) was added pyrrolidine (8.74 mg, 122.92 μmol, 10.21 μL) and C_{S2}CO₃ (80.10 mg, 245.85 μmol), then the mixture was stirred at 70° C. for 12 hrs. LCMS showed the reaction was complete. The reaction mixture was filtered and concentrated under reduced pressure to give a crude product. The residue was purified by prep-HPLC (HPLC, TFA condition; column: Phenomenex luna C18 100*40 mm*5 um; mobile phase: [water(0.1% TFA)-ACN]; B %: 1%-45%,8 min). N4,6-dimethyl-N2-[7-(3-pyrrolidin-1-ylpropoxy)-1,3-benzodioxol-5-yl]pyridine-2,4-diamine (23.3 mg, 46.84 μmol, 38.10% yield, TFA, purity: 99.039%) was obtained as a brown solid.

¹H NMR (400 MHz, METHANOL-d₄) δ=6.64-6.45 (m, 2H), 6.30-6.03 (m, 1H), 6.03-5.90 (m, 2H), 5.90-5.61 (m, 1H), 4.30-4.17 (m, 2H), 3.79-3.67 (m, 2H), 3.47-3.38 (m, 2H), 3.21-3.03 (m, 2H), 2.93-2.70 (m, 3H), 2.48-2.27 (m, 3H), 2.26-2.12 (m, 4H), 2.08-2.01 (m, 2H).

### EXAMPLE 34. Synthesis of Compound 148

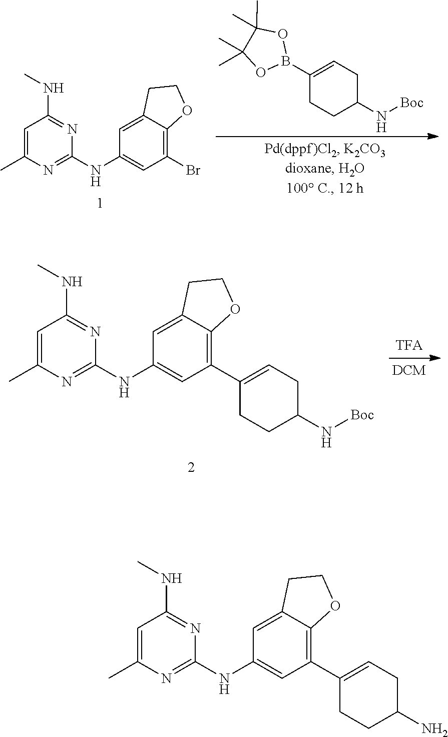 [SMILES: CNc1cc(C)nc(Nc2cc(Br)c3c(c2)CCO3)n1 ; CC(C)(C)OC(=O)NC1CC=C(B2OC(C)(C)C(C)(C)O2)CC1 ; CNc1cc(C)nc(Nc2cc3c(c(C4=CCC(NC(=O)OC(C)(C)C)CC4)c2)OCC3)n1 ; CNc1cc(C)nc(Nc2cc3c(c(C4=CCC(N)CC4)c2)OCC3)n1]

### Step 1. tert-butyl-N-[4-[5-[[4-methyl-6-(methylamino)pyrimidin-2-yl]amino]-2,3-dihydrobenzofuran-7-yl]cyclohex-3-en-1-yl]carbamate

To a solution of tert-butyl N-[4-(4,4,5,5-tetramethyl-1,3,2-dioxaborolan-2-yl)cyclohex-3-en-1-yl]carbamate (151.88 mg, 469.87 μmol), N2-(7-bromo-2,3-dihydrobenzofuran-5-yl)-N4,6-dimethyl-pyrimidine-2,4-diamine (150 mg, 447.50 μmol), K₂CO₃ (185.54 mg, 1.34 mmol) in H₂O (0.3 mL) and dioxane (3 mL) was added Pd(dppf)Cl₂ (32.74 mg, 44.75 μmol). It was stirred at 100° C. for 12 h under N2. LCMS showed starting material was consumed completely and mass of the desired compound. It was concentrated under reduced pressure to give tert-butyl-N-[4-[5-[[4-methyl-6-(methylamino)pyrimidin-2-yl]amino]-2,3-dihydrobenzofuran-7-yl]cyclohex-3-en-1-yl]carbamate (200 mg, crude) as a black solid.

### Step 2. N2-[7-(4-aminocyclohexen-1-yl)-2,3-dihydrobenzofuran-5-yl]-N4,6-dimethyl-pyrimidine-2,4-diamine

To a solution of tert-butyl-N-[4-[5-[[4-methyl-6-(methylamino)pyrimidin-2-yl]amino]-2,3-dihydrobenzofuran-7-yl]cyclohex-3-en-1-yl]carbamate (100 mg, 221.45 μmol) in DCM (1 mL) was added TFA (0.5 mL). It was stirred at 20° C. for 1 h. LCMS showed starting material was consumed completely and mass of the desired compound. It was purified by prep-HPLC (TFA condition, column: Phenomenex luna C18 100*40 mm*5 um; mobile phase: [water(0.1% TFA)-ACN]; B %: 1%-45%,8 min) to give N2-[7-(4-aminocyclohexen-1-yl)-2,3-dihydrobenzofuran-5-yl]-N4,6-dimethyl-pyrimidine-2,4-diamine (11.5 mg, 32.72 μmol, 14.78% yield)(100.0% purity, TFA salt) as a pale yellow solid.

¹H NMR (400 MHz, METHANOL-d₄) δ ppm 7.35-7.41 (m, 1H) 7.24 (br s, 1H) 6.23 (br s, 1H) 5.97 (s, 1H) 4.62 (br t, J=8.74 Hz, 2H) 3.47 (br s, 1H) 3.26 (br t, J=8.62 Hz, 2H) 2.99 (s, 3H) 2.67 (br s, 3H) 2.27-2.37 (m, 4H) 2.20 (br d, J=10.88 Hz, 1H) 1.80-1.92 (m, 1H)

### EXAMPLE 35. Synthesis of Compound 149

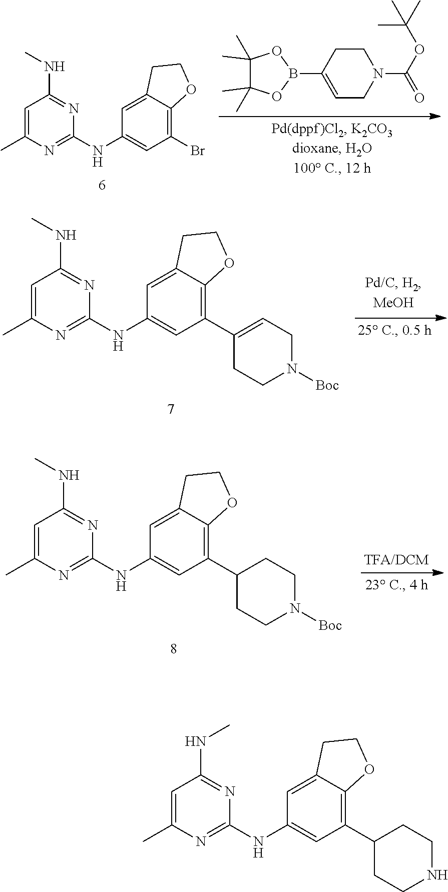

### Step 1. Tert-butyl 4-[5-[[4-methyl-6-(methylamino)pyrimidin-2-yl]amino]-2,3-dihydrobenzo furan-7-yl]-3,6-dihydro-2H-pyridine-1-carboxylate

To a solution of N2-(7-bromo-2,3-dihydrobenzofuran-5-yl)-N4,6-dimethyl-pyrimidine-2,4-diamine (150 mg, 447.50 mol) in H₂O (0.3 mL) and dioxane (3 mL) was added tert-butyl 4-(4,4,5,5-tetramethyl-1,3,2-dioxaborolan-2-yl)-3,6-dihydro-2H-pyridine-1-carboxylate (166.04 mg, 537.00 mol) and K₂CO₃ (185.54 mg, 1.34 mmol), then it was added Pd(dppf)Cl₂ (32.74 mg, 44.75 μmol) under N₂ atmosphere. It was stirred at 100° C. for 12 hours under N₂ atmosphere. LCMS showed starting material was consumed completely and mass of the desired compound. Then it was concentrated under reduced pressure to give tert-butyl 4-[5-[[4-methyl-6-(methylamino) pyrimidin-2-yl]amino]-2,3-dihydrobenzofuran-7-yl]-3,6-dihydro-2H-pyridine-1-carboxylate (195 mg, crude) as a black solid.

### Step 2. Tert-butyl-4-[5-[[4-methyl-6-(methylamino)pyrimidin-2-yl]amino]-2,3-dihydrobenzofuran-7-yl]piperidine-1-carboxylate

To a solution of tert-butyl 4-[5-[[4-methyl-6-(methylamino) pyrimidin-2-yl]amino]-2,3-dihydrobenzofuran-7-yl]-3,6-dihydro-2H-pyridine-1-carboxylate (130 mg, 297.12 mol) in MeOH (30 mL) was added Pd/C (200 mg, 10% purity) under N2. The suspension was degassed under vacuum and purged with H₂ several times. It was stirred at 25° C. for 0.5 hours. LCMS showed starting material was consumed completely and mass of the desired compound. Then it was filtered and concentrated under reduced pressure to give tert-butyl 4-[5-[[4-methyl-6-(methylamino)pyrimidin-2-yl]amino]-2,3-dihydrobenzofuran-7-yl]piperidine-1-carboxylate (160 mg, crude) as a yellow gum.

### Step 3. N4,6-dimethyl-N2-[7-(4-piperidyl)-2,3-dihydrobenzofuran-5-yl]pyrimidine-2,4-diamine

To a solution of tert-butyl 4-[5-[[4-methyl-6-(methylamino) pyrimidin-2-yl]amino]-2,3-dihydrobenzofuran-7-yl]piperidine-1-carboxylate (160 mg, 364.01 μmol) in DCM (2 mL) and TFA (1 mL) was stirred at 23° C. for 4 hours. LCMS showed starting material was consumed completely and mass of the desired compound. Then it was concentrated under reduced pressure to give a residue. It was purified by prep-HPLC (TFA condition, column: Phenomenex Luna C18 100*40 mm*5 um; mobile phase: [water(TFA)-ACN]; B %: 1%-45%,8 min) to give N4,6-dimethyl-N2-[7-(4-piperidyl)-2,3-dihydrobenzofuran-5-yl]pyrimidine-2,4-diamine (39.3 mg, 115.78 μmol, 31.81% yield) (100% purity, TFA salt) as a white solid.

¹H NMR (400 MHz, METHANOL-d₄) δ ppm 2.05-2.12 (m, 4H) 2.31 (s, 3H) 2.99 (s, 3H) 3.04 (br dd, J=15.59, 7.52 Hz, 1H) 3.11-3.20 (m, 2H) 3.27 (t, J=8.62 Hz, 2H) 3.52 (br d, J=12.71 Hz, 2H) 4.58-4.68 (m, 2H) 5.97 (s, 1H) 7.16 (s, 1H) 7.40 (s, 1H). MS (ESI): m/z=340.1 [M+H]⁺

### EXAMPLE 36. Synthesis of Compounds 150 and 151

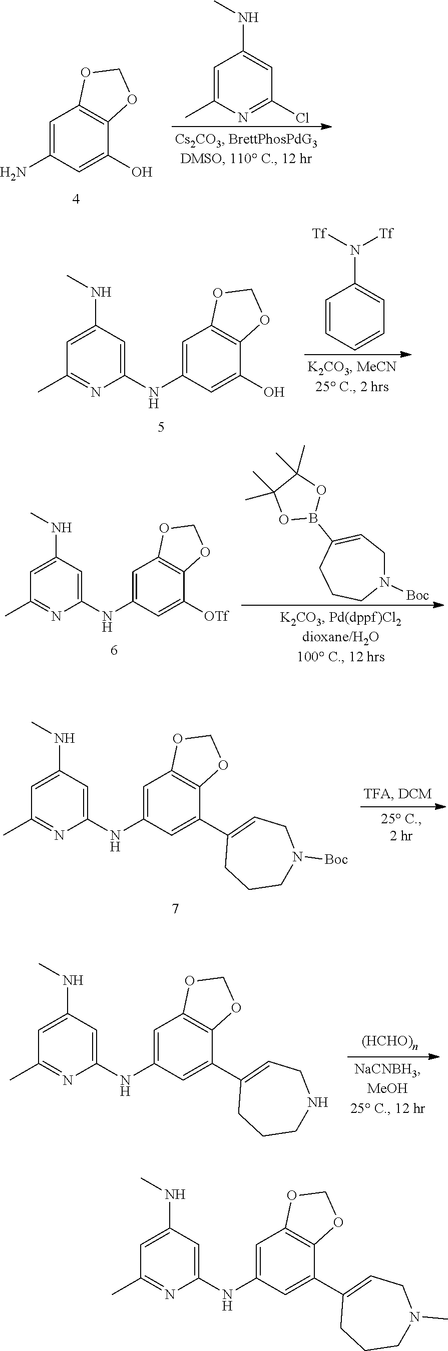

### Step 1. 6-[[6-methyl-4-(methylamino)-2-pyridyl]amino]-1,3-benzodioxol-4-ol

To a mixture of 6-amino-1,3-benzodioxol-4-ol (20 mg, 130.60 μmol) in DMSO (2 mL) was added 2-chloro-N,6-dimethyl-pyridin-4-amine (20.45 mg, 130.60 μmol), C_{S2}CO₃ (127.66 mg, 391.81 mol) and BrettPhosPdG₃ (23.68 mg, 26.12 mol), then the mixture was stirred at 110° C. for 12 hr under N₂ atmosphere. LCMS showed the reaction was complete. The twenty-two batches of the reaction mixture were mixed together for work up. The reaction mixture was diluted with H₂O 50 mL and extracted with EtOAc 300 mL (100 mL*3). The combined organic layers were washed with brine 100 mL, dried over Na₂SO₄, filtered and concentrated under reduced pressure to give a residue. The residue was purified by prep-HPLC (TFA condition; column: Phenomenex luna C18 100*40 mm*5 um; mobile phase: [water(0.1% TFA)-ACN]; B %: 1%-45%,8 min). to give 6-[[6-methyl-4-(methylamino)-2-pyridyl]amino]-1,3-benzodioxol-4-ol (90 mg, crude) as a brown solid.

### Step 2. [6-[[6-methyl-4-(methylamino)-2-pyridyl]amino]-1,3-benzodioxol-4-yl]trifluoromethanesulfonate

A mixture of 6-[[6-methyl-4-(methylamino)-2-pyridyl]amino]-1,3-benzodioxol-4-ol (80 mg, 292.73 μmol),1,1,1-trifluoro-N-phenyl-N-(trifluoromethylsulfonyl)methanesulfonamide (156.87 mg, 439.10 mol), K₂CO₃ (80.91 mg, 585.47 μmol) in MeCN (10 mL) was stirred at 25° C. for 2 h. LCMS showed the reaction was complete. The reaction mixture was filtered and concentrated under reduced pressure to give [6-[[6-methyl-4-(methylamino)-2-pyridyl]amino]-1,3-benzodioxol-4-yl]trifluoromethanesulfonate (115 mg, crude) as brown oil.

### Step 3. tert-butyl5-[6-[[6-methyl-4-(methylamino)-2-pyridyl]amino]-1,3-benzodioxol-4-yl]-2,3,4,7-tetrahydroazepine-1-carboxylate

To a mixture of [6-[[6-methyl-4-(methylamino)-2-pyridyl]amino]-1,3-benzodioxol-4-yl]trifluoromethanesulfonate (115 mg, 283.71 μmol) in H₂O (0.3 mL) and dioxane (3 mL) was added tert-butyl 5-(4,4,5,5-tetramethyl-1,3,2-dioxaborolan-2-yl)-2,3,4,7-tetrahydroazepine-1-carboxylate (110.04 mg, 340.45 mol), Pd(dppf)Cl₂ (20.76 mg, 28.37 mol) and K₂CO₃ (78.42 mg, 567.41 mol), then the mixture was stirred at 100° C. for 12 hr under N₂ atmosphere. LCMS showed the reaction was complete. The reaction mixture was filtered and concentrated under reduced pressure to give a crude product. The residue was purified by prep-TLC (DCM/MeOH=10:1, Rf=0.4) to give tert-butyl 5-[6-[[6-methyl-4-(methylamino)-2-pyridyl]amino]-1,3-benzodioxol-4-yl]-2,3,4,7-tetrahydroazepine-1-carboxylate (130 mg, crude) as brown oil.

### Step 4. N4,6-dimethyl-N2-[7-(2,3,4,7-tetrahydro-1H-azepin-5-yl)-1,3-benzodioxol-5-yl pyridine-2,4-diamine

A mixture of tert-butyl 5-[6-[[6-methyl-4-(methylamino)-2-pyridyl]amino]-1,3-benzodioxol-4-yl]-2,3,4,7-tetrahydroazepine-1-carboxylate (130 mg, 287.26 μmol) in DCM (2 mL) and TFA (1 mL) was stirred at 25° C. for 1 h. LCMS showed the reaction was complete. The reaction mixture was filtered and concentrated under reduced pressure to give a crude product. A part of the residue was purified by prep-HPLC (TFA condition; column: Phenomenex luna C18 100*40 mm*5 um; mobile phase: [water(0.1% TFA)-ACN]; B %: 1%-45%,8 min). to give N4,6-dimethyl-N2-[7-(2,3,4,7-tetrahydro-1H-azepin-5-yl)-1,3-benzodioxol-5-yl]pyridine-2,4-diamine (14.6 mg, 31.37 μmol, 10.92% yield, TFA) as yellow gum. And other part of the residue was concentrated to give N4,6-dimethyl-N2-[7-(2,3,4,7-tetrahydro-1H-azepin-5-yl)-1,3-benzodioxol-5-yl]pyridine-2,4-diamine (70 mg, crude) as brown oil.

¹H NMR (400 MHz, METHANOL-d₄) δ=6.89-6.64 (m, 2H), 6.21 (br t, J=6.2 Hz, 1H), 6.19-5.94 (m, 3H), 5.89-5.57 (m, 1H), 4.01-3.86 (m, 2H), 3.55-3.41 (m, 2H), 2.95-2.87 (m, 2H), 2.97-2.69 (m, 5H), 2.48-2.24 (m, 3H), 2.16-1.91 (m, 2H)

### Step 5. N4,6-dimethyl-N2-[7-(1-methyl-2,3,4,7-tetrahydroazepin-5-yl)-1,3-benzodioxol-5-yl]pyridine-2,4-diamine

To a solution of N4,6-dimethyl-N2-[7-(2,3,4,7-tetrahydro-1H-azepin-5-yl)-1,3-benzodioxol-5-yl]pyridine-2,4-diamine (50 mg, 141.87 μmol) in MeOH (2 mL) was added DIEA adjust to pH=7-8, AcOH was added to above solution to adjust pH=5-6. (HCHO)ₙ(21.30 mg, 709.36 mol) and sodium cyanoboranuide (17.83 mg, 283.74 μmol) was added to above solution. Then the mixture was stirred at 25° C. for 12 hr. LCMS showed the reaction was complete. The reaction mixture was filtered and concentrated under reduced pressure to give a crude product. The residue was purified by prep-HPLC (TFA condition; column: Phenomenex luna C18 100*40 mm*5 um; mobile phase: [water(0.1% TFA)-ACN]; B %: 1%-45%,8 min) to give N4,6-dimethyl-N2-[7-(1-methyl-2,3,4,7-tetrahydroazepin-5-yl)-1,3-benzodioxol-5-yl]pyridine-2,4-diamine (34.3 mg, 71.54 μmol, 50.42% yield, TFA, purity: 95.601%) as yellow gum.

¹H NMR (400 MHz, METHANOL-d₄) δ=6.89-6.69 (m, 2H), 6.16 (br t, J=6.7 Hz, 1H), 6.05 (br s, 3H), 5.89-5.61 (m, 1H), 4.17-3.93 (m, 2H), 3.75-3.62 (m, 1H), 3.49-3.38 (m, 1H), 2.92 (s, 3H), 2.85-2.85 (m, 1H), 2.90-2.66 (m, 4H), 2.44-2.27 (m, 3H), 2.17-1.98 (m, 2H)

### EXAMPLE 37. Synthesis of Compound 152

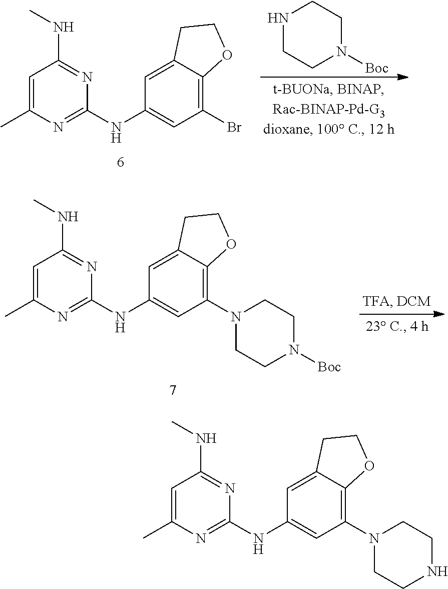 [SMILES: CNc1cc(C)nc(Nc2cc(Br)c3c(c2)CCO3)n1 ; CC(C)(C)OC(=O)N1CCNCC1 ; CNc1cc(C)nc(Nc2cc3c(c(N4CCN(C(=O)OC(C)(C)C)CC4)c2)OCC3)n1 ; CNc1cc(C)nc(Nc2cc3c(c(N4CCNCC4)c2)OCC3)n1]

### Step 1. Tert-butyl 4-[5-[[4-methyl-6-(methylamino) pyrimidin-2-yl]amino]-2,3-dihydrobenzo furan-7-ylpiperazine-1-carboxylate

To a solution of N2-(7-bromo-2,3-dihydrobenzofuran-5-yl)-N4,6-dimethyl-pyrimidine-2,4-diamine (50 mg, 149.17 μmol) in dioxane (1 mL) was added tert-butyl piperazine-1-carboxylate (41.67 mg, 223.75 mol), [1-(2-diphenylphosphanyl-1-naphthyl)-2-naphthyl]-diphenyl-phosphane (19.41 mg, 29.83 mol) and sodium; 2-methylpropan-2-olate (43.01 mg, 447.50 μmol), then it was added Rac-BINAP-Pd-G3 (14.86 mg, 14.92 mol) under N2 atmosphere, it was stirred at 100° C. for 12 hours under N₂ atmosphere. LCMS showed starting material was consumed completely and mass of the desired compound. Then it was concentrated under reduced pressure to give tert-butyl 4-[5-[[4-methyl-6-(methylamino) pyrimidin-2-yl]amino]-2,3-dihydrobenzofuran-7-yl]piperazine-1-carboxylate (65 mg, crude) as a yellow solid.

### Step 2. N4,6-dimethyl-N2-(7-piperazin-1-yl]-2,3-dihydrobenzofuran-5-yl)pyrimidine-2,4-diamine

To a solution of tert-butyl 4-[5-[[4-methyl-6-(methylamino)pyrimidin-2-yl]amino]-2,3-dihydrobenzofuran-7-yl]piperazine-1-carboxylate (65 mg, 147.55 μmol) in TFA (0.5 mL) and DCM (1 mL) was stirred at 23° C. for 4 hours. LCMS showed starting material was consumed and mass of the desired compound. Then it was concentrated under reduced pressure to give a residue. It was purified by prep-HPLC (TFA condition, column: Phenomenex Luna C18 100*40 mm*5 um; mobile phase: [water(TFA)-ACN]; B %: 1%-45%,8 min) to give N4,6-dimethyl-N2-(7-piperazin-1-yl]-2,3-dihydrobenzofuran-5-yl)pyrimidine-2,4-diamine (21.5 mg, 63.16 μmol, 42.80% yield) (98.86% purity, TFA salt) as a yellow gum.

¹H NMR (400 MHz, METHANOL-d₄) δ ppm 2.30 (s, 3H) 3.00 (s, 3H) 3.26 (br t, J=8.62 Hz, 2H) 3.39 (s, 8H) 4.65 (t, J=8.68 Hz, 2H) 5.97 (s, 1H) 6.99 (br s, 1H) 7.14-7.23 (m, 1H). MS (ESI): m/z=341.1 [M+H]⁺

### EXAMPLE 38. Synthesis of Compound 153

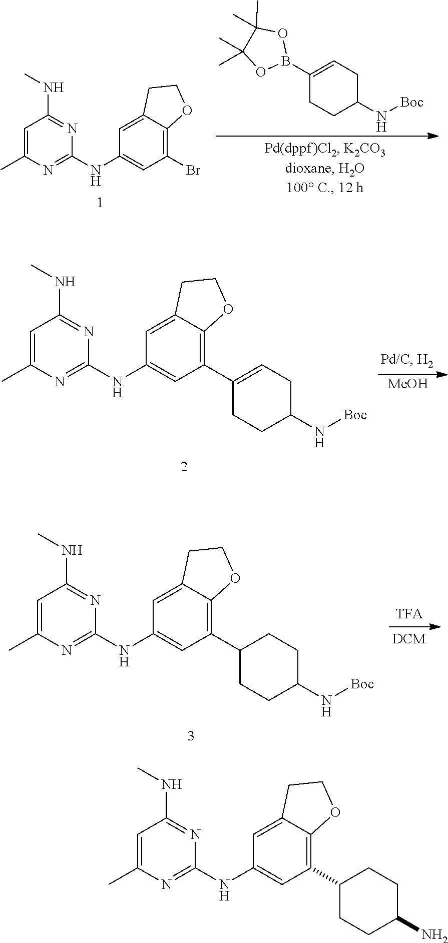

### Step 1. tert-butyl-N-[4-[5-[[4-methyl-6-(methylamino)pyrimidin-2-yl]amino]-2,3-dihydrobenzofuran-7-yl]cyclohex-3-en-1-yl]carbamate

To a solution of tert-butyl-N-[4-(4,4,5,5-tetramethyl-1,3,2-dioxaborolan-2-yl)cyclohex-3-en-1-yl]carbamate (151.88 mg, 469.87 μmol), N2-(7-bromo-2,3-dihydrobenzofuran-5-yl)-N4,6-dimethyl-pyrimidine-2,4-diamine (150 mg, 447.50 μmol), K₂CO₃ (185.54 mg, 1.34 mmol) in H₂O (0.3 mL) and dioxane (3 mL) was added Pd(dppf)Cl₂ (32.74 mg, 44.75 μmol). It was stirred at 100° C. for 12 hrs under N2. LCMS showed starting material was consumed completely and mass of the desired compound. It was concentrated under reduced pressure to give tert-butyl-N-[4-[5-[[4-methyl-6-(methylamino)pyrimidin-2-yl]amino]-2,3-dihydrobenzofuran-7-yl]cyclohex-3-en-1-yl]carbamate (200 mg, crude) as a black solid.

### Step 2. tert-butyl-N-[4-[5-[4-methyl-6-(methylamino)pyrimidin-2-yl]amino]-2,3-dihydrobenzofuran-7-yl]cyclohexyl]carbamate

To a solution of tert-butyl-N-[4-[5-[[4-methyl-6-(methylamino)pyrimidin-2-yl]amino]-2,3-dihydrobenzofuran-7-yl]cyclohex-3-en-1-yl]carbamate (100 mg, 221.45 μmol) in MeOH (50 mL) was added Pd/C (100 mg, 10% purity). It was stirred at 20° C. for 1 h under H₂ (15 PSI). LCMS showed starting material was consumed completely and mass of the desired compound. It was filtered and concentrated under reduced pressure to give tert-butyl N-[4-[5-[[4-methyl-6-(methylamino)pyrimidin-2-yl]amino]-2,3-dihydrobenzofuran-7-yl]cyclohexyl]carbamate (100 mg, crude) was obtained as a yellow oil.

### Step 3. N2-[7-(4-aminocyclohexyl)-2,3-dihydrobenzofuran-5-yl]-N4,6-dimethyl-pyrimidine-2,4-diamine

To a solution of tert-butyl-N-[4-[5-[[4-methyl-6-(methylamino)pyrimidin-2-yl]amino]-2,3-dihydrobenzofuran-7-yl]cyclohexyl]carbamate (100 mg, 220.47 μmol) in DCM (1 mL) was added TFA (0.5 mL). It was stirred at 20° C. for 1 h. LCMS showed starting material was consumed completely and mass of the desired compound. It was concentrated under reduced pressure to give a residue. It was purified by prep-HPLC (TFA condition, column: Phenomenex luna C18 100*40 mm*5 um; mobile phase: [water(0.1% TFA)-ACN]; B %: 1%-45%,8 min) to give N2-[7-(4-aminocyclohexyl)-2,3-dihydrobenzofuran-5-yl]-N4,6-dimethyl-pyrimidine-2,4-diamine (29.2 mg, 82.61 μmol, 37.47% yield)(TFA salt, 98.564% purity) as a white solid and N2-[7-(4-aminocyclohexyl)-2,3-dihydrobenzofuran-5-yl]-N4,6-dimethyl-pyrimidine-2,4-diamine (65.6 mg, 185.59 mol) (TFA salt, 98.923% purity) as a white solid.

¹H NMR (400 MHz, METHANOL-d₄) δ ppm 7.32 (s, 1H) 7.19 (s, 1H) 5.95 (s, 1H) 4.55-4.63 (m, 2H) 3.23 (br t, J=8.68 Hz, 2H) 3.13-3.20 (m, 1H) 2.98 (s, 3H) 2.71-2.80 (m, 1H) 2.29 (s, 3H) 2.17 (br d, J=10.27 Hz, 2H) 2.00 (br d, J=11.98 Hz, 2H) 1.65-1.76 (m, 2H) 1.51-1.63 (m, 2H).

### EXAMPLE 39. Synthesis of Compound 154

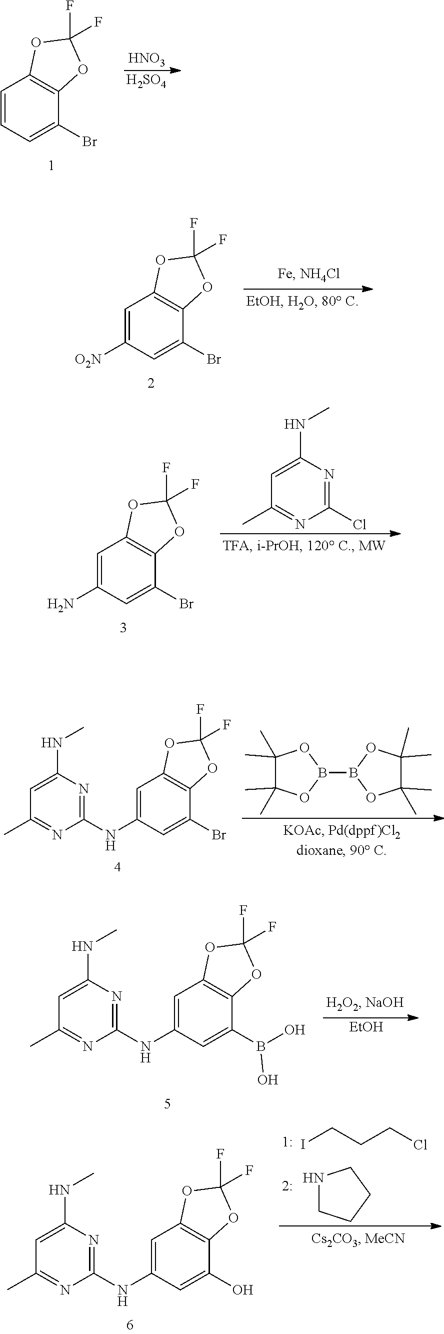

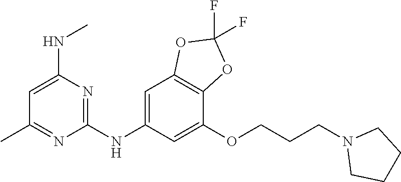 [SMILES: CNc1cc(C)nc(Nc2cc(OCCCN3CCCC3)c3c(c2)OC(F)(F)O3)n1]

### Step 1. 4-bromo-2,2-difluoro-6-nitro-1,3-benzodioxole

A mixture of HNO₃ (0.5 mL)/H₂SO₄ (0.5 mL) was added drop wise to the mixture of 4-bromo-2,2-difluoro-1,3-benzodioxole (500 mg, 2.11 mmol, 1 eq.) in H₂SO₄ (1 mL) at 0° C., then the reaction mixture was stirred at 0° C. for 1 hr. TLC (petroleum ether:ethyl acetate=5:1, Rf=0.75) showed the reaction was complete and 2 new spots were formed. The reaction mixture was added drop wise the ice-water (20 mL), extracted with EtOAc (10 mL*2). The organic layer was dried over Na₂SO₄, concentrated to give a residue. The reside was purified by column chromatography on silica gel (petroleum ether:ethyl acetate=0:1-100; 1) to give 4-bromo-2,2-difluoro-6-nitro-1,3-benzodioxole (100 mg, 354.62 μmol, 16.81% yield) as yellow oil.

### Step 2. 7-bromo-2,2-difluoro-1,3-benzodioxol-5-amine

To a mixture of 4-bromo-2,2-difluoro-6-nitro-1,3-benzodioxole (100 mg, 354.62 mol, 1 eq.) in EtOH (3 mL) and H₂O (0.6 mL) was added Fe (99.02 mg, 1.77 mmol, 5 eq.) and ammonia hydrochloride (189.69 mg, 3.55 mmol, 10 eq.), then the mixture was stirred at 80° C. for 1 h. LCMS showed the reaction was complete mostly and the desired ms was detected. The reaction was filtered and concentrated under reduced pressure to give 7-bromo-2,2-difluoro-1,3-benzodioxol-5-amine (36 mg, crude) as a light yellow solid.

### Step 3. N2-(7-bromo-2,2-difluoro-1,3-benzodioxol-5-yl)-N4,6-dimethyl-pyrimidine-2,4-diamine

To a mixture of 7-bromo-2,2-difluoro-1,3-benzodioxol-5-amine (360 mg, 1.43 mmol, 1 eq.) and 2-chloro-N,6-dimethyl-pyrimidin-4-amine (225.13 mg, 1.43 mmol, 1 eq.) in i-PrOH (2.89 mL) was added HCl (12 M, 119.04 μL, 1 eq.), then the mixture was stirred at 130° C. in the microwave for 1 hrs. LCMS showed the reaction was complete mostly and the desired ms was detected. The reaction was filtered by EtOAc(2 mL*2) to give filter cake. The filter cake was dried in vacuum to give N2-(7-bromo-2,2-difluoro-1,3-benzodioxol-5-yl)-N4,6-dimethyl-pyrimidine-2,4-diamine (380 mg, crude) as a light yellow solid.

### Step 4. [2,2-difluoro-6-[[4-methyl-6-(methylamino)pyrimidin-2-yl]amino]-1,3-benzodioxol-4-yl]boronic acid

To a mixture of N2-(7-bromo-2,2-difluoro-1,3-benzodioxol-5-yl)-N4,6-dimethyl-pyrimidine-2,4-diamine (150 mg, 401.98 μmol, 1 eq.) and 4,4,5,5-tetramethyl-2-(4,4,5,5-tetramethyl-1,3,2-dioxaborolan-2-yl)-1,3,2-dioxaborolane (122.49 mg, 482.38 μmol, 1.2 eq.) in Dioxane (4 mL) was added potassium acetate (78.90 mg, 803.96 μmol, 2 eq.) and cyclopentyl(diphenyl)phosphane;dichloropalladium;iron (29.41 mg, 40.20 μmol, 0.1 eq.), then the mixture was stirred at 90° C. for 12 hrs under N2. LCMS showed the reaction was complete mostly and the desired ms was detected. The reaction was filtered and concentrated under reduced pressure to give a residue. The residue was purified by prep-TLC (dichloromethane/methyl alcohol=12:1, Rf=0.3) to give [2,2-difluoro-6-[[4-methyl-6-(methylamino)pyrimidin-2-yl]amino]-1,3-benzodioxol-4-yl]boronic acid (60 mg, crude) as a black solid.

### Step 5. 2,2-difluoro-6-[[4-methyl-6-(methylamino)pyrimidin-2-yl]amino]-1,3-benzodioxol-4-ol

[2,2-Difluoro-6-[[4-methyl-6-(methylamino)pyrimidin-2-yl]amino]-1,3-benzodioxol-4-yl]boronic acid (60 mg, 177.48 μmol, 1 eq.) was dissolved in EtOH (4 mL), and then to the mixture was added H₂O₂ (40.25 mg, 354.95 mol, 30% purity, 2 eq.) at 0° C., and then the mixture was stirred at 25° C. for 2 h. LCMS showed starting material was consumed completely and mass of the desired compound. The reaction was added saturated sodium sulfite solution (4 mL), then was added water (4 mL), then extracted with ethyl acetate (2 mL*3) and dried over anhydrous sodium sulfate, filtered and concentrated under reduced pressure to give 2,2-difluoro-6-[[4-methyl-6-(methylamino)pyrimidin-2-yl]amino]-1,3-benzodioxol-4-ol (50 mg, crude) as a brown solid.

### Step 6. N2-[2,2-difluoro-7-(3-pyrrolidin-1-ylpropoxy)-1,3-benzodioxol-5-yl]-N4,6-dimethyl-pyrimidine-2,4-diamine

A mixture of 2,2-difluoro-6-[[4-methyl-6-(methylamino)pyrimidin-2-yl]amino]-1,3-benzodioxol-4-ol (40 mg, 128.93 μmol, 1 eq.) and 1-chloro-3-iodo-propane (52.71 mg, 257.85 mol, 27.69 μL, 2 eq.) in MeCN (2 mL) was added dicesium;carbonate (126.02 mg, 386.78 μmol, 3 eq.), then the reaction was stirred at 50° C. for 12 hrs. LCMS showed the reaction was complete and the desired ms was detected. The mixture was used for the next step directly without workup. N2-[7-(3-chloropropoxy)-2,2-difluoro-1,3-benzodioxol-5-yl]-N4,6-dimethyl-pyrimidine-2,4-diamine (50 mg, crude) in MeCN (2 mL) was used for next step directly.

To a mixture of N2-[7-(3-chloropropoxy)-2,2-difluoro-1,3-benzodioxol-5-yl]-N4,6-dimethyl-pyrimidine-2,4-diamine (50 mg, 129.27 μmol, 1 eq.) in MeCN (1 mL) was added NaI (23.25 mg, 155.13 μmol, 6.34 μL, 1.2 eq.) and pyrrolidine (45.97 mg, 646.36 μmol, 53.70 μL, 5 eq.), then the mixture was stirred at 80° C. for 12 hrs. LCMS showed the reaction was complete mostly and the desired ms was detected. The reaction was filtered and concentrated under reduced pressure to give a residue. The crude product was purified by prep-HPLC(TFA condition:column: Phenomenex Luna 80*30 mm*3 um; mobile phase:water(TFA)-ACN]; B %: 10%-45%,8 min) to give N2-[2,2-difluoro-7-(3-pyrrolidin-1-ylpropoxy)-1,3-benzodioxol-5-yl]-N4,6-dimethyl-pyrimidine-2,4-diamine (8.4 mg, 19.93 μmol, 15.42% yield) as a white solid.

¹H NMR (400 MHz, METHANOL-d₄) δ=7.43 (br s, 1H), 7.06 (br s, 1H), 6.02 (br s, 1H), 4.30 (t, J=5.5 Hz, 2H), 3.83-3.61 (m, 2H), 3.46-3.40 (m, 2H), 3.27-3.07 (m, 2H), 3.01 (s, 3H), 2.42-2.25 (m, 5H), 2.24-1.79 (m, 4H). MS (ESI): m/z=422.1 [M+H]⁺

### EXAMPLE 40. Synthesis of Compound 155

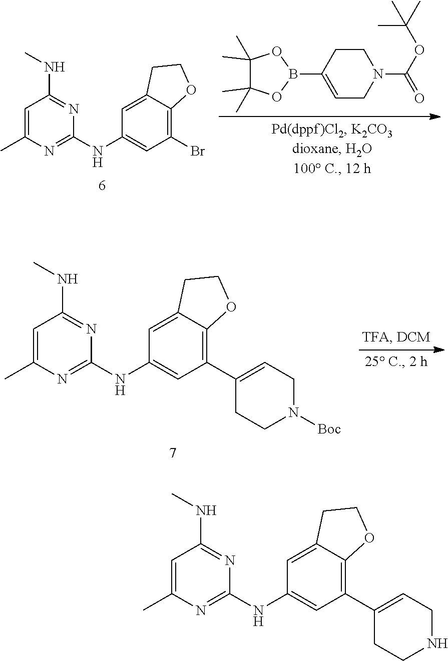

### Step 1. Tert-butyl 4-[5-{{4-methyl-6-(methylamino)pyrimidin-2-yl]amino]-2,3-dihydrobenzo-furan-7-yl]-3,6-dihydro-2H-pyridine-1-carboxylate

To a solution of N2-(7-bromo-2,3-dihydrobenzofuran-5-yl)-N4,6-dimethyl-pyrimidine-2,4-diamine (150 mg, 447.50 mol) in H₂O (0.3 mL) and dioxane (3 mL) was added tert-butyl 4-(4,4,5,5-tetramethyl-1,3,2-dioxaborolan-2-yl)-3,6-dihydro-2H-pyridine-1-carboxylate (166.04 mg, 537.00 mol) and K₂CO₃ (185.54 mg, 1.34 mmol), then it was added Pd(dppf)Cl₂ (32.74 mg, 44.75 μmol) under N₂ atmosphere. It was stirred at 100° C. for 12 hours under N₂ atmosphere. LCMS showed starting material was consumed completely and mass of the desired compound. Then it was concentrated under reduced pressure to give tert-butyl 4-[5-[[4-methyl-6-(methylamino) pyrimidin-2-yl]amino]-2,3-dihydrobenzofuran-7-yl]-3,6-dihydro-2H-pyridine-1-carboxylate (195 mg, crude) as a black solid.

### Step 2. N4,6-dimethyl-N2-[7-(1, 2, 3,6-tetrahydropyridin-4-yl]-2,3-dihydrobenzofuran-5-yl]pyrimidine-2,4-diamine

The solution of tert-butyl 4-[5-[[4-methyl-6-(methylamino) pyrimidin-2-yl]amino]-2,3-dihydrobenzofuran-7-yl]-3,6-dihydro-2H-pyridine-1-carboxylate (65 mg, 148.56 μmol) in DCM (1 mL) and TFA (0.5 mL) was stirred at 25° C. for 2 hours. LCMS showed starting material was consumed completely and mass of the desired compound. Then it was concentrated under reduced pressure to give a residue. It was purified by prep-HPLC (TFA condition, column: Phenomenex Luna C18 100*40 mm*5 um; mobile phase: [water(TFA)-ACN]; B %: 1%-45%,8 min) to give N4,6-dimethyl-N2-[7-(1,2,3,6-tetrahydropyridin-4-yl)-2,3-dihydrobenzofuran-5-yl]pyrimidine-2,4-diamine (14.7 mg, 43.57 μmol, 29.33% yield) (100% purity, TFA salt) as a white solid.

¹H NMR (400 MHz, METHANOL-d₄) δ ppm 2.30 (s, 3H) 2.85 (br d, J=1.63 Hz, 2H) 2.94-3.02 (m, 3H) 3.27 (br t, J=8.63 Hz, 2H) 3.46 (t, J=6.07 Hz, 2H) 3.87 (br d, J=2.38 Hz, 2H) 4.65 (t, J=8.69 Hz, 2H) 5.97 (s, 1H) 6.41 (br s, 1H) 7.29 (s, 1H) 7.43 (s, 1H).

MS (ESI): m/z=338.1 [M+H]⁺

### EXAMPLE 41. Synthesis of Compound 156

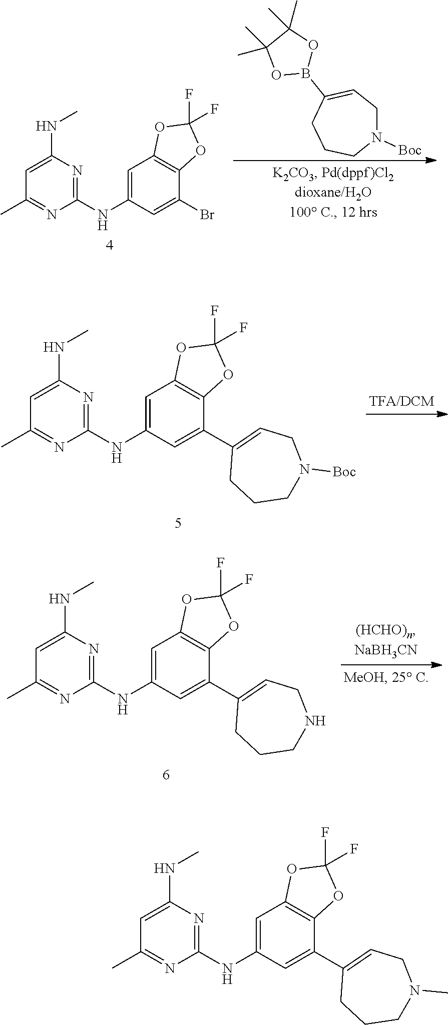

### Step]. 5-[2,2-difluoro-6-[[4-methyl-6-(methylamino)pyrimidin-2-yl]amino-],3-benzodioxol-4-yl]-2,3,4,7-tetrahydroazepine-1-carboxylate

To a mixture of N2-(7-bromo-2,2-difluoro-1,3-benzodioxol-5-yl)-N4,6-dimethyl-pyrimidine-2,4-diamine (150 mg, 401.98 μmol, 1 eq.) and tert-butyl 5-(4,4,5,5-tetramethyl-1,3,2-dioxaborolan-2-yl)-2,3,4,7-tetrahydroazepine-1-carboxylate (129.93 mg, 401.98 μmol, 1 eq.) in dioxane (1.96 mL) and H₂O (196.18 μL) was added tripotassium carbonate (111.11 mg, 803.96 μmol, 48.52 μL, 2 eq.) and cyclopentyl(diphenyl)phosphane;dichloropalladium;iron (29.41 mg, 40.20 μmol, 0.1 eq.), then the mixture was stirred at 100° C. for 12 hrs under N2. LCMS showed the reaction was complete and the desired ms was detected. The reaction was filtered and concentrated under reduced pressure to give a residue. The residue was purified by prep-TLC(methylene dichloride/methyl alcohol=20:1, Rf=0.4) to give tert-butyl 5-[2,2-difluoro-6-[[4-methyl-6-(methylamino)pyrimidin-2-yl]amino]-1,3-benzodioxol-4-yl]-2,3,4,7-tetrahydroazepine-1-carboxylate (208 mg, crude) as a black solid.

### Step 2. N2-[2,2-difluoro-7-(2,3,4,7-tetrahydro-1H-azepin-5-yl)-1,3-benzodioxol-5-yl]-N4,6-dimethyl-pyrimidine-2,4-diamine

To a mixture of tert-butyl 5-[2,2-difluoro-6-[[4-methyl-6-(methylamino)pyrimidin-2-yl]amino]-1,3-benzodioxol-4-yl]-2,3,4,7-tetrahydroazepine-1-carboxylate (208 mg, 424.91 μmol, 1 eq.) in DCM (3 mL) was added TFA (0.5 mL), then the mixture was stirred at 25° C. for 2 hrs. LCMS showed the reaction was complete and the desired ms was detected. The reaction was concentrated under reduced pressure to give N2-[2,2-difluoro-7-(2,3,4,7-tetrahydro-1H-azepin-5-yl)-1,3-benzodioxol-5-yl]-N4,6-dimethyl-pyrimidine-2,4-diamine (300 mg, crude) as a yellow oil.

### Step 3. N2-[2,2-difluoro-7-(1-methyl-2,3,4,7-tetrahydroazepin-5-yl)-1,3-benzodioxol-5-yl]-N4,6-dimethyl-pyrimidine-2,4-diamine

To a solution of N2-[2,2-difluoro-7-(2,3,4,7-tetrahydro-1H-azepin-5-yl)-1,3-benzodioxol-5-yl]-N4,6-dimethyl-pyrimidine-2,4-diamine (70 mg, 179.76 μmol, 1 eq.) in MeOH (3 mL) was added DIEA adjust to pH=7-8, then the mixture was added (HCHO)ₙ(53.98 mg, 1.80 mmol, 10 eq.) and NaBH₃CN (22.59 mg, 359.53 μmol, 2 eq.) then the mixture was stirred at 25° C. 12 hrs. LCMS showed the reaction was complete and the desired ms was detected. The reaction was filtered and concentrated under reduced pressure to give a residue. The crude product was purified by prep-HPLC(TFA condition:column:Phenomenex luna C18 100*40 mm*5 um; mobile phase:water(TFA)-ACN]; B %: 1%-45%,8 min) to give N2-[2,2-difluoro-7-(1-methyl-2,3,4,7-tetrahydroazepin-5-yl)-1,3-benzodioxol-5-yl]-N4,6-dimethyl-pyrimidine-2,4-diamine (19 mg, 36.79 μmol, 20.47% yield, TFA) as a white solid.

¹H NMR (400 MHz, METHANOL-d₄) δ=7.80-7.72 (m, 1H), 7.28 (d, J=1.8 Hz, 1H), 6.24 (t, J=6.6 Hz, 1H), 6.03 (s, 1H), 4.21-3.99 (m, 2H), 3.79-3.62 (m, 1H), 3.54-3.41 (m, 1H), 3.00 (s, 3H), 2.97-2.95 (m, 3H), 2.94-2.88 (m, 2H), 2.32 (s, 3H), 2.24-2.01 (m, 2H). MS (ESI): m/z=404.1 [M+H]⁺

### EXAMPLE 42. Synthesis of Compound 157

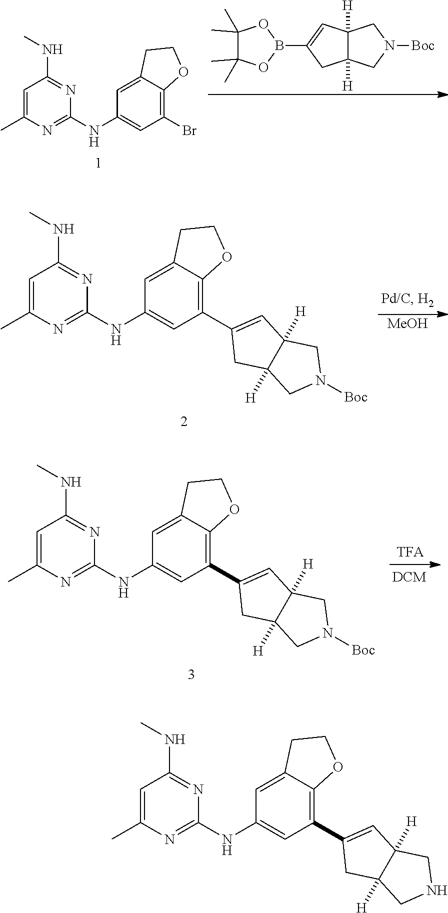

### Step 1. tert-butyl-5-[5-[4-methyl-6-(methylamino)pyrimidin-2-yl]amino]-2,3-dihydro benzofuran-7-yl]-3,3a,6,6a-tetrahydro-1H-cyclopenta[c]pyrrole-2-carboxylate

To a solution of N2-(7-bromo-2,3-dihydrobenzofuran-5-yl)-N4,6-dimethyl-pyrimidine-2,4-diamine (100 mg, 298.33 mol), tert-butyl 5-(4,4,5,5-tetramethyl-1,3,2-dioxaborolan-2-yl)-3,3a,6,6a-tetrahydro-1H-cyclopenta[c]pyrrole-2-carboxylate (110.02 mg, 328.16 μmol), K₂CO₃ (123.69 mg, 894.99 mol) in H₂O (0.3 mL) and dioxane (3 mL) was added Pd(dppf)Cl₂ (21.83 mg, 29.83 mol). It was stirred at 100° C. for 12 h under N2. LCMS showed starting material remained and mass of the desired compound. It was concentrated under reduced pressure to give a residue. It was purified by prep-TLC(petroleum ether:ethyl acetate=2:1) to give tert-butyl-5-[5-[[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate (70 mg, 151.00 μmol, 50.62% yield) as a yellow oil.

### Step 2. tert-butyl-5-[5-[[4-methyl-6-(methylamino)pyrimidin-2-yl]amino]-2,3-dihydrobenzo furan-7-yl]-3,3a,4,5,6,6a-hexahydro-1H-cyclopenta[c]pyrrole-2-carboxylate

To a solution of tert-butyl 5-[5-[[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate (70 mg, 151.00 μmol) in MeOH (20 mL) was added Pd/C (70 mg, 10% purity). It was stirred at 20° C. for 1 h under H₂ (15 Psi). LCMS showed starting material was consumed completely and mass of the desired compound. It was filtered and concentrated under reduced pressure to give tert-butyl 5-[5-[[4-methyl-6-(methylamino)pyrimidin-2-yl]amino]-2,3-dihydrobenzofuran-7-yl]-3,3a,4,5,6,6a-hexahydro-1H-cyclopenta[c]pyrrole-2-carboxylate (70 mg, crude) was obtained as a yellow oil.

### Step 3. benzyl N-(7-bromo-2,3-dihydrobenzofuran-5-yl)carbamate

To a solution of tert-butyl 5-[5-[[4-methyl-6-(methylamino)pyrimidin-2-yl]amino]-2,3-dihydrobenzofuran-7-yl]-3,3a,4,5,6,6a-hexahydro-1H-cyclopenta[c]pyrrole-2-carboxylate (70.00 mg, 150.35 μmol) in DCM (1 mL) was added TFA (0.5 mL). It was stirred at 20° C. for 1 h. LCMS showed starting material was consumed completely and mass of the desired compound. It was purified by prep-HPLC (TFA condition, column: Phenomenex luna C18 100*40 mm*5 um; mobile phase: [water(0.1% TFA)-ACN]; B %: 1%-45%,8 min) to give N2-[7-(1,2,3,3a,4,5,6,6a-octahydrocyclopenta[c]pyrrol-5-yl)-2,3-dihydrobenzofuran-5-yl]-N4,6-dimethyl-pyrimidine-2,4-diamine (6.9 mg, 18.88 μmol, 12.56% yield) (TFA salt, 100.0% purity) as a yellow solid.

¹H NMR (400 MHz, METHANOL-d₄) δ ppm 7.20 (br s, 1H) 7.04 (s, 1H) 5.83 (s, 1H) 4.46-4.51 (m, 2H) 3.23-3.28 (m, 2H) 3.10-3.17 (m, 5H) 2.87-2.92 (m, 2H) 2.85 (s, 3H) 2.22-2.28 (m, 2H) 2.17 (s, 3H) 1.52 (td, J=12.13, 8.76 Hz, 2H)

### EXAMPLE 43. Synthesis of Compound 158

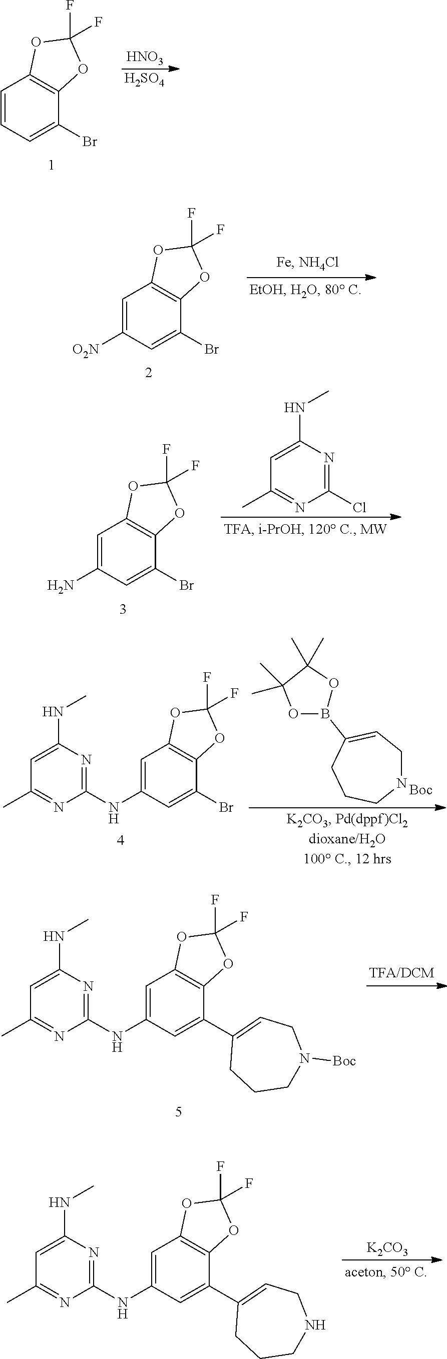

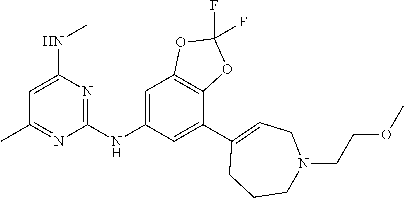

### Step 1. 4-bromo-2,2-difluoro-6-nitro-],3-benzodioxole

A mixture of HNO₃ (0.5 mL) and H₂SO₄ (0.5 mL) was added drop wise to the mixture of 4-bromo-2,2-difluoro-1,3-benzodioxole (500 mg, 2.11 mmol, 1 eq.) in H₂SO₄ (1 mL) at 0° C., then the reaction mixture was stirred at 0° C. for 1 hr. TLC (petroleum ether:ethyl acetate=5:1, Rf=0.75) showed the reaction was complete and 2 new spots were formed. The reaction mixture was added dropwise the ice-water (20 mL), extracted with EtOAc (10 mL*2). The organic layer was dried over Na₂SO₄, concentrated to give a residue. The reside was purified by column chromatography on silica gel (petroleum ether:ethyl acetate=0:1-100; 1) to give 4-bromo-2,2-difluoro-6-nitro-1,3-benzodioxole (100 mg, 354.62 μmol, 16.81% yield) as yellow oil.

### Step 2. 7-bromo-2,2-difluoro-1,3-benzodioxol-5-amine

To a mixture of 4-bromo-2,2-difluoro-6-nitro-1,3-benzodioxole (100 mg, 354.62 mol, 1 eq.) in EtOH (3 mL) and H₂O (0.6 mL) was added Fe (99.02 mg, 1.77 mmol, 5 eq.) and ammonia hydrochloride (189.69 mg, 3.55 mmol, 10 eq.), then the mixture was stirred at 80° C. for 1 h. LCMS showed the reaction was complete mostly and the desired ms was detected. The reaction was filtered and concentrated under reduced pressure to give 7-bromo-2,2-difluoro-1,3-benzodioxol-5-amine (36 mg, crude) as a light yellow solid.

### Step 3. N2-(7-bromo-2,2-difluoro-],3-benzodioxol-5-yl)-N4,6-dimethyl-pyrimidine-2,4-diamine

To a mixture of 7-bromo-2,2-difluoro-1,3-benzodioxol-5-amine (36 mg, 142.85 mol, 1 eq.) and 2-chloro-N,6-dimethyl-pyrimidin-4-amine (22.51 mg, 142.85 μmol, 1 eq.) in i-PrOH (3 mL) was added HCl (12 M, 11.90 μL, 1 eq.), then the mixture was stirred at 130° C. in the microwave for 1 hrs. LCMS showed the reaction was complete mostly and the desired ms was detected. The reaction was concentrated under reduced pressure to give N2-(7-bromo-2,2-difluoro-1,3-benzodioxol-5-yl)-N4,6-dimethyl-pyrimidine-2,4-diamine (60 mg, crude) as a light yellow solid.

### Step 4. 5-[2,2-difluoro-6-[[4-methyl-6-(methylamino)pyrimidin-2-yl]amino-],3-benzodioxol-4-yl]-2,3,4,7-tetrahydroazepine-1-carboxylate

To a mixture of N2-(7-bromo-2,2-difluoro-1,3-benzodioxol-5-yl)-N4,6-dimethyl-pyrimidine-2,4-diamine (150 mg, 401.98 μmol, 1 eq.) and tert-butyl 5-(4,4,5,5-tetramethyl-1,3,2-dioxaborolan-2-yl)-2,3,4,7-tetrahydroazepine-1-carboxylate (129.93 mg, 401.98 μmol, 1 eq.) in dioxane (1.96 mL) and H₂O (196.18 μL) was added tripotassium carbonate (111.11 mg, 803.96 μmol, 48.52 μL, 2 eq.) and cyclopentyl(diphenyl)phosphane;dichloropalladium;iron (29.41 mg, 40.20 μmol, 0.1 eq.), then the mixture was stirred at 100° C. for 12 hrs under N2. LCMS showed the reaction was complete and the desired ms was detected. The reaction was filtered and concentrated under reduced pressure to give a residue. The residue was purified by prep-TLC(methylene dichloride/methyl alcohol=20:1, Rf=0.4) to give tert-butyl 5-[2,2-difluoro-6-[[4-methyl-6-(methylamino)pyrimidin-2-yl]amino]-1,3-benzodioxol-4-yl]-2,3,4,7-tetrahydroazepine-1-carboxylate (208 mg, crude) as a black solid.

### Step 5. N2-[2,2-difluoro-7-(2,3,4,7-tetrahydro-1H-azepin-5-yl)-1,3-benzodioxol-5-yl]-N4,6-dimethyl-pyrimidine-2,4-diamine

To a mixture of tert-butyl 5-[2,2-difluoro-6-[[4-methyl-6-(methylamino)pyrimidin-2-yl]amino]-1,3-benzodioxol-4-yl]-2,3,4,7-tetrahydroazepine-1-carboxylate (208 mg, 424.91 μmol, 1 eq.) in DCM (3 mL) was added TFA (0.5 mL), then the mixture was stirred at 25° C. for 2 hrs. LCMS showed the reaction was complete and the desired ms was detected. The reaction was concentrated under reduced pressure to give N2-[2,2-difluoro-7-(2,3,4,7-tetrahydro-1H-azepin-5-yl)-1,3-benzodioxol-5-yl]-N4,6-dimethyl-pyrimidine-2,4-diamine (300 mg, crude) as a yellow oil.

### Step 6. N2-[2,2-difluoro-7-[1-(2-methoxyethyl)-2,3,4,7-tetrahydroazepin-5-yl]-1,3-benzodioxol-5-yl]-N4,6-dimethyl-pyrimidine-2,4-diamine

To a solution of N2-[2,2-difluoro-7-(2,3,4,7-tetrahydro-1H-azepin-5-yl)-1,3-benzodioxol-5-yl]-N4,6-dimethyl-pyrimidine-2,4-diamine (70 mg, 179.76 μmol, 1 eq.) and 1-bromo-2-methoxy-ethane (49.97 mg, 359.53 mol, 33.81 μL, 2 eq.) in Acetone (3 mL) was added tripotassium carbonate (74.53 mg, 539.29 μmol, 32.55 μL, 3 eq.), the mixture was stirred at 50° C. for 12 h. LCMS showed the reaction was complete and the desired ms was detected. The reaction was filtered and concentrated under reduced pressure to give a residue. The crude product was purified by prep-HPLC(TFA condition:column: Phenomenex luna C18 100*40 mm*5 um; mobile phase:water(TFA)-ACN]; B %: 1%-45%,8 min) to give N2-[2,2-difluoro-7-[1-(2-methoxyethyl)-2,3,4,7-tetrahydroazepin-5-yl]-1,3-benzodioxol-5-yl]-N4,6-dimethyl-pyrimidine-2,4-diamine (18.6 mg, 41.57 μmol, 23.12% yield) as a white solid.

¹H NMR (400 MHz, METHANOL-d₄) δ=7.86-7.83 (m, 1H), 7.82-7.74 (m, 1H), 7.78 (d, J=1.8 Hz, 1H), 7.29 (d, J=1.9 Hz, 1H), 6.22 (t, J=6.7 Hz, 1H), 6.03 (s, 1H), 4.23-4.08 (m, 2H), 3.74 (t, J=5.0 Hz, 2H), 3.71-3.54 (m, 2H), 3.44 (s, 5H), 3.01 (s, 3H), 2.93 (br s, 2H), 2.32 (s, 3H), 2.23-2.04 (m, 2H). MS (ESI): m/z=448.1 [M+H]⁺

### EXAMPLE 44. Synthesis of Compound 170 and Compound 169

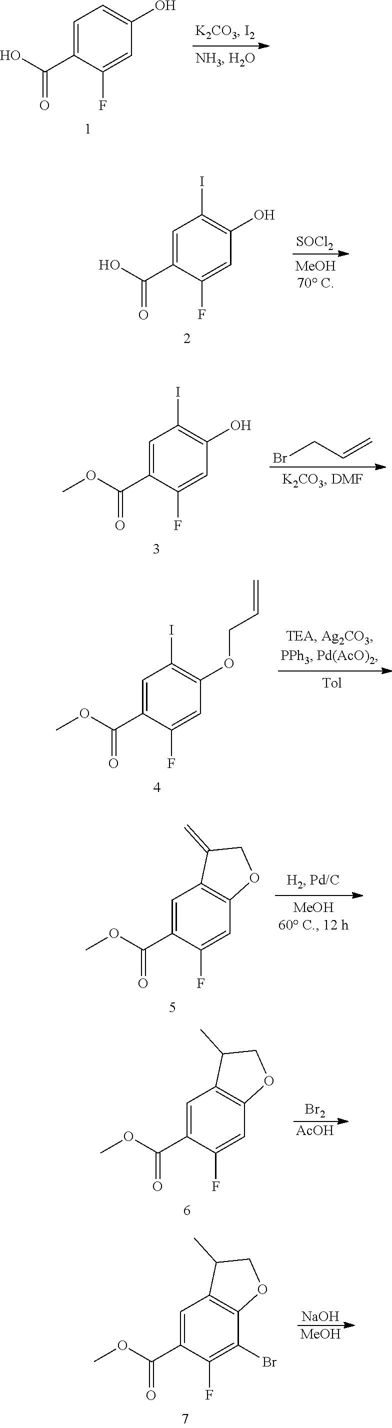

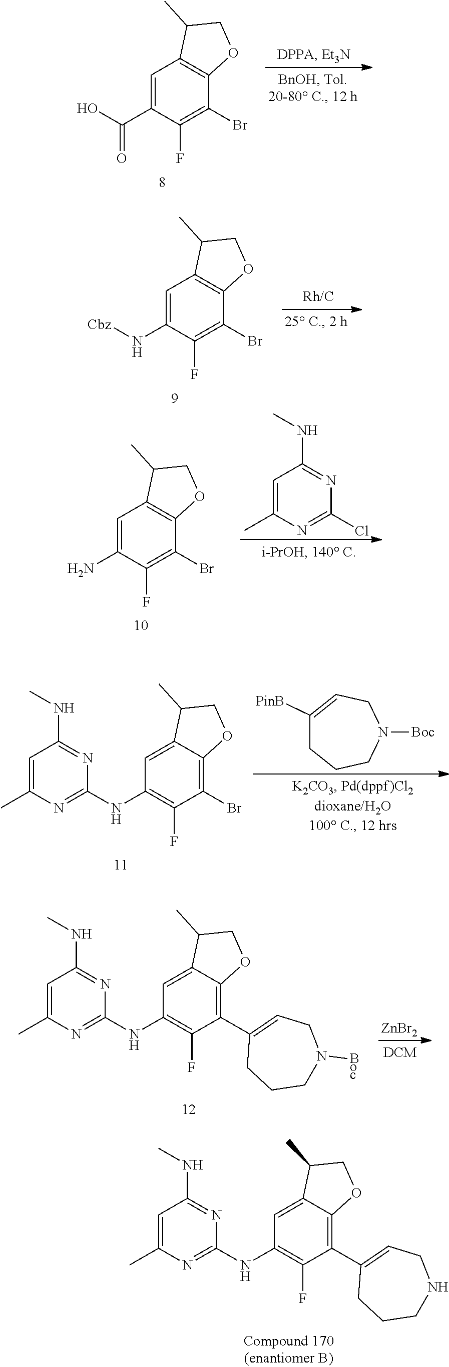

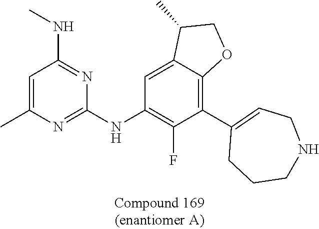 [SMILES: CNc1cc(C)nc(Nc2cc3c(c(C4=CCNCCC4)c2F)OC[C@H]3C)n1]

### Step 1. 2-fluoro-4-hydroxy-5-iodo-benzoic acid

K₂CO₃ (33.20 g, 240.21 mmol) was dissolved in NH₃·H₂O (200 mL), 12 (20.32 g, 80.07 mmol) was added, then the mixture was stirred at about 25° C. for about 1 hr. Then 2-fluoro-4-hydroxy-benzoic acid (12.5 g, 80.07 mmol) was added to the above mixture. After addition, the reaction mixture was stirred at about 25° C. for about 2 hrs. LCMS showed the reaction was complete mostly and the desired mass was detected. The four batches of the reaction mixture were mixed together for work up. The reaction mixture was acidified by HCl (2 M) till pH=1. The reaction mixture was added to water (1 L), extracted with EtOAc (1 L*3). The combined organic layers were washed with 1 L of brine, dried over Na₂SO₄, filtered and concentrated under reduced pressure to afford 2-fluoro-4-hydroxy-5-iodo-benzoic acid (106 g, crude).

### Step 2. methyl 2-fluoro-4-hydroxy-5-iodo-benzoate

To a mixture of 2-fluoro-4-hydroxy-5-iodo-benzoic acid (11.2 g, 39.72 mmol) in MeOH (500 mL) was added SOCl₂ (30 mL) at about 0° C., then the mixture was stirred at about 70° C. for about 2 hrs. TLC (Petroleum ether:Ethyl acetate=5:1) showed the starting material was consumed and new spots were formed. The two reactions were worked up together. The mixture was concentrated in vacuo to give crude product. The residue was purified by column chromatography (silica gel, petroleum ether/ethyl acetate=1/0 to 100/1). Methyl 2-fluoro-4-hydroxy-5-iodo-benzoate (49 g, crude) was obtained.

### Step 3. methyl 4-allyloxy-2-fluoro-5-iodo-benzoate

Methyl 2-fluoro-4-hydroxy-5-iodo-benzoate (10 g, 33.78 mmol) was dissolved in DMF (100 mL), and then to the mixture was added K₂CO₃ (9.34 g, 67.56 mmol) and 3-bromoprop-1-ene (4.50 g, 37.16 mmol), then the mixture was stirred at about 50° C. for about 12 hrs. TLC (Petroleum ether:Ethyl acetate=5:1) showed the starting material was consumed and new spots was formed. The mixture was concentrated in vacuo. The residue was purified by flash chromatography (silica gel, petroleum ether/ethyl acetate=1/0 to 100/1) to give methyl 4-allyloxy-2-fluoro-5-iodo-benzoate (8.5 g, crude).

### Step 4. methyl 6-fluoro-3-methylene-benzofuran-5-carboxylate

To a solution of methyl 4-allyloxy-2-fluoro-5-iodo-benzoate (5 g, 14.88 mmol), TEA (3.01 g, 29.75 mmol, 4.15 mL), Ag₂CO₃ (8.20 g, 29.75 mmol) and PPh₃ (780.38 mg, 2.98 mmol) in toluene (100 mL) was added Pd(AcO)₂ (333.99 mg, 1.49 mmol), then the mixture was stirred at about 70° C. for about 12 h under an atmosphere of nitrogen. TLC (petroleum ether:ethyl acetate=10:1) indicated starting material was consumed completely, and three major new spots were detected. The reaction was concentrated under reduced pressure to give a residue. The residue was purified by flash chromatography (silica gel, petroleum ether/ethyl acetate=1/0 to 19/1) to give methyl 6-fluoro-3-methylene-benzofuran-5-carboxylate (2 g, crude).

### Step 5. methyl 6-fluoro-3-methyl-2,3-dihydrobenzofuran-5-carboxylate

To a mixture of methyl 6-fluoro-3-methylene-benzofuran-5-carboxylate (3.3 g, 15.85 mmol) in MeOH (400 mL) was added Pd/C (1.65 g, 13.59 mmol) under H₂ at about 25° C., then the mixture was stirred at about 60° C. for about 12 hr under H₂ (15 psi). LCMS showed the reaction was complete mostly and the desired mass was detected. The reaction mixture was filtered and concentrated, methyl 6-fluoro-3-methyl-2,3-dihydrobenzofuran-5-carboxylate (3 g, crude) was obtained.

### Step 6. methyl 7-bromo-6-fluoro-3-methyl-2,3-dihydrobenzofuran-5-carboxylate

To a mixture of methyl 6-fluoro-3-methyl-2,3-dihydrobenzofuran-5-carboxylate (3 g, 14.27 mmol) in AcOH (20 mL) was added dropwise Br₂ (6.84 g, 42.82 mmol) at about 0° C., then the mixture was stirred at about 20° C. for about 12 hrs. LCMS showed the reaction was complete and the desired mass was detected. The residue was purified by flash chromatography (silica gel, petroleum ether/ethyl acetate), the reaction mixture was concentrated under reduced pressure to give methyl 7-bromo-6-fluoro-3-methyl-2,3-dihydrobenzofuran-5-carboxylate (2.8 g, crude).

### Step 7. 7-bromo-6-fluoro-3-methyl-2,3-dihydrobenzofuran-5-carboxylic acid

To a solution of methyl 7-bromo-6-fluoro-3-methyl-2,3-dihydrobenzofuran-5-carboxylate (2.8 g, 9.69 mmol) in H₂O (9 mL) and MeOH (30 mL) was added NaOH (774.77 mg, 19.37 mmol, 363.74 μL). It was stirred at about 20° C. for about 12 hrs. LCMS showed the reaction was complete and the desired mass was detected. The reaction mixture was acidified by HCl (2 M) till pH=1. The reaction mixture was filtered to get a cake, the cake was the product. The filtrate was added to water (60 ml), extracted with EtOAc (60 ml*3). The combined organic layers were dried over Na₂SO₄, filtered and concentrated under reduced pressure to afford 7-bromo-6-fluoro-3-methyl-2,3-dihydrobenzofuran-5-carboxylic acid (2.3 g, crude).

### Step 8. benzyl N-(7-bromo-6-fluoro-3-methyl-2,3-dihydrobenzofuran-5-yl)carbamate

To a solution of 7-bromo-6-fluoro-3-methyl-2,3-dihydrobenzofuran-5-carboxylic acid (2.3 g, 8.36 mmol) in toluene (30 mL) was added DPPA (2.44 g, 10.03 mmol), TEA (2.54 g, 25.08 mmol, 3.50 mL). The reaction mixture was stirred at about 20° C. for about 1 h. Then BnOH (1.13 g, 25.08 mmol) was added to the above mixture. After addition the reaction mixture was stirred at about 80° C. for about 12 hrs. TLC (Petroleum ether:Ethyl acetate=3:1) showed the starting material was consumed and a new spot was formed. The residue was purified by flash chromatography (silica gel, petroleum ether/ethyl acetate=1/0 to 20/1 to 5/1) to give benzyl N-(7-bromo-6-fluoro-3-methyl-2,3-dihydrobenzofuran-5-yl)carbamate (3 g, crude).

### Step 9. 7-bromo-6-fluoro-3-methyl-2,3-dihydrobenzofuran-5-amine

Benzyl N-(7-bromo-6-fluoro-3-methyl-2,3-dihydrobenzofuran-5-yl)carbamate (1 g, 2.63 mmol) was dissolved in EtOH (100 mL), and then to the mixture was added Rh/C (500 mg, 211.97 μmol, 5% purity), and then the mixture was stirred at about 25° C. for about 2 hrs under H₂ (15 psi). LCMS showed the starting material remained and the desired mass was detected. The reaction mixture was filtered and the filtrate was concentrated to give a residue. The residue was purified by flash chromatography (silica gel, petroleum ether/ethyl acetate=1/0 to 10/1) to give 7-bromo-6-fluoro-3-methyl-2,3-dihydrobenzofuran-5-amine (270 mg, crude).

### Step 10. N2-(7-bromo-6-fluoro-3-methyl-2,3-dihydrobenzofuran-5-yl)-N4,6-dimethyl-pyrimidine-2,4-diamine

To a solution of 7-bromo-6-fluoro-3-methyl-2,3-dihydrobenzofuran-5-amine (270 mg, 1.10 mmol) and 2-chloro-N,6-dimethyl-pyrimidin-4-amine (172.92 mg, 1.10 mmol) in i-PrOH (1 mL) was added TFA (235.42 mg, 2.06 mmol, 159.07 μL), then the mixture was stirred at about 140° C. for about 2 hrs. LCMS showed the starting material remained and the desired mass was detected. The reaction was concentrated under reduced pressure to give N2-(7-bromo-6-fluoro-3-methyl-2,3-dihydrobenzofuran-5-yl)-N4,6-dimethyl-pyrimidine-2,4-diamine (190 mg, crude).

### Step 11. tert-butyl5-[6-fluoro-3-methyl-5-({4-me thyl-6-(methylamino)pyrimidin-2-yl]amino]-2,3-dihydrobenzofuran-7-yl]-2,3,4,7-tetrahydroazepine-1-carboxylate

To a mixture of N2-(7-bromo-6-fluoro-3-methyl-2,3-dihydrobenzofuran-5-yl)-N4,6-dimethyl-pyrimidine-2,4-diamine (100 mg, 272.32 μmol), tert-butyl 5-(4,4,5,5-tetramethyl-1,3,2-dioxaborolan-2-yl)-2,3,4,7-tetrahydroazepine-1-carboxylate (88.02 mg, 272.32 μmol) in H₂O (0.3 mL) and dioxane (3 mL) was added K₂CO₃ (56.45 mg, 408.48 mol) and cyclopentyl(diphenyl)phosphane;dichloromethane;dichloropalladium;iron (22.24 mg, 27.23 mol) under N₂ atmosphere. The reaction mixture was stirred at about 100° C. for about 12 hrs under N₂ atmosphere. LCMS showed the reaction was complete and the desired mass was detected. The reaction mixture was concentrated to give a residue. The residue was purified by prep-TLC (SiO₂, DCM/MeOH=10:1). tert-butyl 5-[6-fluoro-3-methyl-5-[[4-methyl-6-(methylamino) pyrimidin-2-yl]amino]-2,3-dihydrobenzofuran-7-yl]-2,3,4,7-tetrahydroazepine-1-carboxylate (100 mg, crude) was obtained.

### Step 12. N4,6-dimethyl-N2-[rel-(3R)-6-fluoro-3-methyl-7-(2,3,4,7-tetrahydro-1H-azepin-5-yl)-2,3-dihydrobenzofuran-5-yl]pyrimidine-2,4-diamine and N4,6-dimethyl-N2-[rel-(3S)-6-fluoro-3-methyl-7-(2,3,4,7-tetrahydro-1H-azepin-5-yl)-2,3-dihydrobenzofuran-5-yl]pyrimidine-2,4-diamine

To a solution of tert-butyl 5-[6-fluoro-3-methyl-5-[[4-methyl-6-(methylamino) pyrimidin-2-yl]amino]-2,3-dihydrobenzofuran-7-yl]-2,3,4,7-tetrahydroazepine-1-carboxylate (100 mg, 206.79 mol) in DCM (2 mL) was added ZnBr₂ (232.85 mg, 1.03 mmol), then stirred at about 30° C. for about 12 hrs. LCMS showed starting material remained and mass of the desired compound. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give a mixture of Compound 170. The mixture was separated by SFC (basic condition, column: DAICEL CHIRALCEL OX (250 mm*30 mm*10 um); mobile phase: [0.1% NH₃H₂O MeOH; B %: 60%-60%, 15 min) to give N4,6-dimethyl-N2-[rel-(3R)-6-fluoro-3-methyl-7-(2,3,4,7-tetrahydro-1H-azepin-5-yl)-2,3-dihydrobenzofuran-5-yl]pyrimidine-2,4-diamine (20.4 mg, 53.20 μmol) (purity: 100%, ee:100%) and N4,6-dimethyl-N2-[rel-(3S)-6-fluoro-3-methyl-7-(2,3,4,7-tetrahydro-1H-azepin-5-yl)-2,3-dihydrobenzofuran-5-yl]pyrimidine-2,4-diamine (20.9 mg, 54.50 μmol) (purity:100%, ee:97.3%).

¹H NMR (400 MHz, CD₃OD, 298 K) δ: 7.88 (br d, J=4.5 Hz, 1H), 5.99 (t, J=5.8 Hz, 1H), 5.80 (s, 1H), 4.67 (t, J=8.8 Hz, 1H), 4.05 (dd, J=7.6, 8.5 Hz, 1H), 3.47 (d, J=5.8 Hz, 2H), 3.37-3.25 (m, 4H), 3.16-3.05 (m, 2H), 2.88 (s, 3H), 2.64-2.55 (m, 2H), 2.16 (s, 3H), 1.93-1.77 (m, 2H), 1.31 (d, J=6.9 Hz, 3H), 1.15 (d, J=6.1 Hz, 1H)

¹H NMR (400 MHz, CD₃OD, 298 K) δ: 7.88 (br d, J=3.1 Hz, 1H), 5.99 (t, J=5.7 Hz, 1H), 5.80 (s, 1H), 4.68 (t, J=8.8 Hz, 1H), 4.05 (dd, J=7.6, 8.6 Hz, 1H), 3.47 (d, J=5.9 Hz, 2H), 3.36-3.27 (m, 7H), 3.16-3.06 (m, 2H), 2.88 (s, 3H), 2.64-2.56 (m, 2H), 2.16 (s, 3H), 1.91-1.81 (m, 2H), 1.31 (d, J=6.9 Hz, 3H)

### EXAMPLE 45. Synthesis of Compound 179

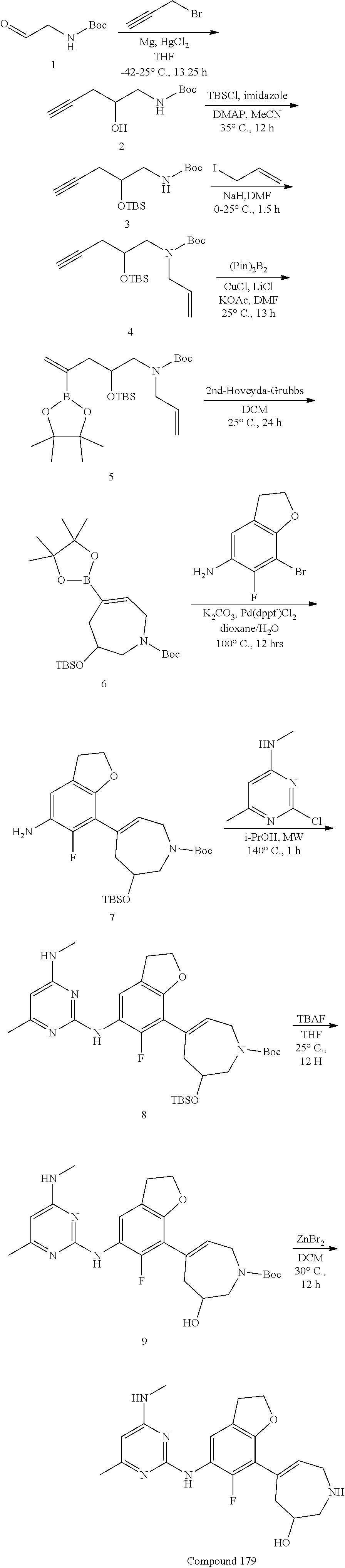

### Step 1. tert-butyl N-(2-hydroxypent-4-ynyl)carbamate

Mg (3.7 g, 152.23 mmol) was flame dried under vacuum, suspended in THF (55 mL), and treated with HgCl₂ (150 mg, 552.49 mol). The mixture was stirred at about 25° C. for about 30 min then cooled to about 4° C., and 3-bromoprop-1-yne (1.17 g, 7.85 mmol) was added. The mixture was stirred for about 15 min at about 25° C. and a rise in temperature was observed. The solution was maintained at about 4° C. and the remainder of the 3-bromoprop-1-yne (13.45 g, 113.08 mmol) was added dropwise. The mixture was stirred at about 0° C. for an additional about 30 min, and then the mixture was transferred via cannula to a flask cooled to about −42° C. tert-butyl N-(2-oxoethyl)carbamate (5 g, 31.41 mmol) in THF (10 mL) was added dropwise at about −40° C. The reaction mixture was warmed to about 25° C. for about 12 h. TLC (petroleum ether:ethyl acetate=1:1) indicated Reactant 1 was consumed completely, and one major new spot was detected. The mixture was poured into a cold saturated NH₄Cl solution at about 0° C., producing vigorous bubbling. The aqueous layer was extracted with 3*30 mL of EtOAc. The organic layers were combined and dried over Na₂SO₄, filtered, and concentrated in vacuo. The residue was purified via flash chromatography silica gel (petroleum ether/ethyl acetate=50/1 to 5/1 to 1/1). tert-butyl N-(2-hydroxypent-4-ynyl)carbamate (5 g, 25.09 mmol) was obtained.

### Step 2. tert-butyl N-[2-[tert-butyl(dimethyl)sily]oxypent-4-ynyl]carbamate

The tert-butyl N-(2-hydroxypent-4-ynyl)carbamate (100 mg, 501.89 mol) was dissolved in MeCN (2 mL), tert-butyl-chloro-dimethyl-silane (90.77 mg, 602.27 mol, 112.07 μL), Imidazole (51.25 mg, 752.84 μmol), and N,N-dimethyl]pyridin-4-amine (73.58 mg, 602.27 μmol) were then added at about 35° C. after about 12 h. TLC (petroleum ether:ethyl acetate=2:1, Rf=0.8) indicated Reactant 1 was consumed completely, and one major new spot was detected. The mixture was concentrated at about 45° C., and dissolved in NaHCO₃ (10 mL), and then extracted with EtOAc (5 mL*2), and then the organic phase was concentrated in vacuum. The residue was purified by flash chromatography (silica gel, petroleum ether/ethyl acetate=0/1 to 10/1). tert-butyl N-[2-[tert-butyl(dimethyl)silyl]oxypent-4-ynyl]carbamate (110 mg, 350.87 μmol) was obtained.

### Step 3. tert-butyl N-allyl-N-[2-[tert-butyl(dimethyl)sily]oxypent-4-ynyl]carbamate

tert-butyl N-[2-[tert-butyl(dimethyl)silyl]oxypent-4-ynyl]carbamate (110 mg, 350.87 μmol) was dissolved in DMF (2 mL), and then to the mixture was added NaH (16.84 mg, 421.04 mol, 60% purity) at about 0° C., and then the mixture was stirred at about 0° C. for about 30 min, and then to the mixture was added 3-iodoprop-1-ene (70.73 mg, 421.04 mol, 38.50 μL) at about 0° C., the mixture was stirred at about 25° C. for about 1 h. TLC (petroleum ether:ethyl acetate=3:1) indicated Reactant 1 was consumed completely, and one major new spot was detected. LCMS showed reactant 1 was consumed completely and desired mass was detected. The reaction was quenched by sat. aq. NH₄Cl (10 mL), and then extracted with EtOAc (4 mL*3), and then the mixture was concentrated in vacuo. The residue was purified by prep-TLC (SiO₂, petroleum ether:ethyl acetate=3:1). tert-butyl N-allyl-N-[2-[tert-butyl(dimethyl)silyl]oxypent-4-ynyl]carbamate (57 mg, 161.21 mol) was obtained.

### Step 4. tert-butyl N-allyl-N-[2-[tert-butyl(dimethyl)silyl]oxy-4-(4,4,5,5-tetramethyl-1,3,2-dioxaborolan-2-yl)pent-4-enyl]carbamate

A mixture of LiCl (8.20 mg, 193.46 μmol, 3.97 μL) and CuCl (19.15 mg, 193.46 mol) in DMF (2 mL) was stirred at about 25° C. for about 1 hr under N2, then to the solution was added tert-butyl N-allyl-N-[2-[tert-butyl(dimethyl)silyl]oxypent-4-ynyl]carbamate (57 mg, 161.21 μmol), 4,4,5,5-tetramethyl-2-(4,4,5,5-tetramethyl-1,3,2-dioxaborolan-2-yl)-1,3,2-dioxaborolane (49.13 mg, 193.46 μmol) and KOAc (54.57 mg, 193.46 μmol) and stirred at about 25° C. for about 12 hrs under N2. LCMS showed reactant 1 was consumed completely and desired mass was detected. TLC (Petroleum ether:Ethyl acetate=20:1) showed most of the starting material was consumed and a new spot was formed. The reaction was quenched by NH₄Cl (20 mL), extracted with EtOAc (5 mL*3). The organic layer was dried over Na₂SO₄, concentrated to give the crude product. The residue was purified by prep-TLC (SiO₂, Petroleum ether/Ethyl acetate=20:1). tert-butyl N-allyl-N-[2-[tert-butyl(dimethyl)silyl]oxy-4-(4,4,5,5-tetramethyl-1,3,2-dioxaborolan-2-yl)pent-4-enyl]carbamate (20 mg, 41.53 μmol) was obtained.

### Step 5. tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate

tert-butyl N-allyl-N-[2-[tert-butyl(dimethyl)silyl]oxy-4-(4,4,5,5-tetramethyl-1,3,2-dioxaborolan-2-yl)pent-4-enyl]carbamate (1 g, 2.08 mmol) was dissolved in DCM (150 mL), and then to the mixture was added Hoveyda-Grubbs Catalyst 2nd Generation (65.06 mg, 103.83 mol), and then the mixture was stirred at about 25° C. for about 24 h. TLC (petroleum ether:ethyl acetate=10:1) indicated Reactant 1 was consumed completely, and one major new spot was detected. The mixture was concentrated in vacuum. The residue was purified by flash chromatography (silica gel, petroleum ether/ethyl acetate=1/0 to 20/1). tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (580 mg, crude) was obtained.

### Step 6. tert-butyl 5-(5-amino-6-fluoro-2,3-dihydrobenzofuran-7-yl)-3-[tert-butyl(dimethyl)silyl]oxy-2,3,4,7-tetrahydroazepine-1-carboxylate

To a solution of tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (260 mg, 573.33 μmol) and 7-bromo-6-fluoro-2,3-dihydrobenzofuran-5-amine (102.34 mg, 441.02 μmol) in dioxane (20 mL) and H₂O (2 mL) was added cyclopentyl(diphenyl)phosphane;dichloropalladium;iron (32.27 mg, 44.10 μmol) and tripotassium;carbonate (121.90 mg, 882.04 μmol), and then the reaction mixture was stirred at about 100° C. for about 12 h under N2. LCMS showed Reactant 1 remained and desired mass was detected. The reaction was concentrated under reduced pressure to give a residue. The residue was purified by prep-TLC (SiO₂, Petroleum ether/Ethyl acetate=1:1). tert-butyl 5-(5-amino-6-fluoro-2,3-dihydrobenzofuran-7-yl)

-3-[tert-butyl(dimethyl)silyl]oxy-2,3,4,7-tetrahydroazepine-1-carboxylate (60 mg, crude) was obtained.

### Step 7. tert-butyl 3-[tert-butyl(dimethyl)silyl]oxy-5-[6-fluoro-5-[4-methyl-6-(methylamino)pyrimidin-2-yl]amino]-2,3-dihydrobenzo furan-7-yl]-2,3,4,7-tetrahydroazepine-1-carboxylate

To a mixture of 2-chloro-N,6-dimethyl-pyrimidin-4-amine (19.75 mg, 125.35 μmol) in i-PrOH (2 mL) was added tert-butyl 5-(5-amino-6-fluoro-2,3-dihydrobenzofuran-7-yl)-3-[tert-butyl(dimethyl)silyl]oxy-2,3,4,7-tetrahydroazepine-1-carboxylate (60.00 mg, 125.35 μmol), then the mixture was stirred at about 140° C. for about 1 hr under microwave condition. LCMS showed the reaction was complete and the desired mass was detected. The reaction was concentrated under reduced pressure to give tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (105 mg, crude).

### Step 8. tert-butyl 5-[6-fluoro-5-[[4-methyl-6-(methylamino)pyrimidin-2-yl]amino]-2,3-dihydrobenzofuran-7-yl]-3-hydroxy-2,3,4,7-tetrahydroazepine-1-carboxylate

To a solution of tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (100 mg, 166.72 μmol) was added TBAF (4 mL) (1M in THF), then it was stirred at about 25° C. for about 12 hrs. LCMS showed starting material remained and desired mass was detected. The residue was purified by prep-TLC (SiO₂, Ethyl acetate/MeOH=5:1). tert-butyl 5-[6-fluoro-5-[[4-methyl-6-(methylamino)pyrimidin-2-yl]amino]-2,3-dihydrobenzofuran-7-yl]-3-hydroxy-2,3,4,7-tetrahydroazepine-1-carboxylate (200 mg, crude) was obtained.

### Step 9. 5-[6-fluoro-5-[[4-methyl-6-(methylamino)pyrimidin-2-yl]amino]-2,3-dihydrobenzo furan-7-yl]-2,3,4,7-tetrahydro-1H-azepin-3-ol

A solution of tert-butyl 5-[6-fluoro-5-[[4-methyl-6-(methylamino)pyrimidin-2-yl]amino]-2,3-dihydrobenzofuran-7-yl]-3-hydroxy-2,3,4,7-tetrahydroazepine-1-carboxylate (100 mg, 205.95 μmol) and dibromozinc (231.90 mg, 1.03 mmol) in DCM (2 mL) was stirred at about 30° C. for about 12 hrs. LCMS showed the reaction was complete and the desired mass was detected. The reaction was concentrated under reduced pressure to give a residue. The crude product was purified by prep-HPLC (TFA condition:column:Phenomenex Luna 80*30 mm*3 um; mobile phase:water(TFA)-ACN; B %: 1%-3%,8 min). Compound 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (1.9 mg, 3.81 μmol, TFA) was obtained.

¹H NMR (400 MHz, METHANOL-d₄) δ: 7.45-7.31 (m, 1H), 6.18 (s, 1H), 6.02-5.94 (m, 1H), 4.73-4.62 (m, 2H), 4.22-4.11 (m, 1H), 3.95-3.85 (m, 2H), 3.60-3.45 (m, 1H), 3.37-3.32 (m, 1H), 3.28-3.22 (m, 2H), 3.02-2.86 (m, 5H), 2.41-2.25 (m, 3H).

MS (ESI): m/z=386.1 [M+H]

### EXAMPLE 46. Synthesis of Compound 202

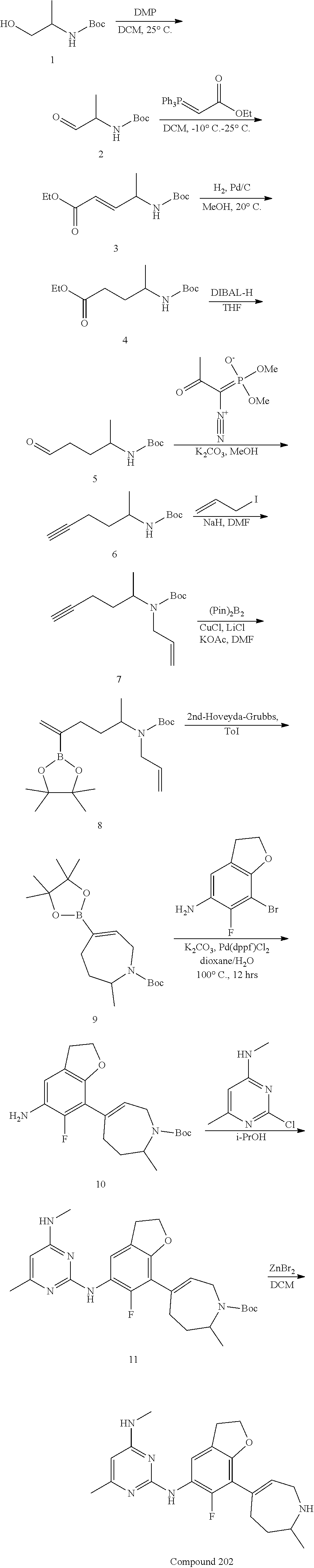

### Step 1. tert-butyl N-(1-methyl-2-oxo-ethyl)carbamate

To a solution of (1,1-diacetoxy-3-oxo-1,2-benziodoxol-1-yl) acetate (12.3 g, 29.00 mmol) in DCM (65 mL) was added tert-butyl N-(2-hydroxy-1-methyl-ethyl)carbamate (5 g, 28.53 mmol) at about 0° C., then stirring at about 25° C. for about 12 hrs. TLC (Petroleum ether: Ethyl acetate=1:1) showed the starting material was consumed and a new spot was formed. The reaction mixture was quenched by Na₂O₃S₂ (10 mL), extracted with CH₂Cl₂ (10 mL*5). The organic layer was dried over Na₂SO₄, concentrated to give tert-butyl N-(1-methyl-2-oxo-ethyl)carbamate (9 g, crude).

### Step 2. ethyl(E)-4-(tert-butoxycarbonylamino)pent-2-enoate

To a solution of tert-butyl N-(1-methyl-2-oxo-ethyl)carbamate (2.00 g, 11.55 mmol) in DCM (20 mL) was added ethyl 2-(triphenyl-phosphanylidene)acetate (4.02 g, 11.55 mmol) at about 0° C., then stirred at about 25° C. for about 2 hrs. LCMS showed the reaction was complete and the desired mass was detected. The reaction mixture was added to water (10 mL), extracted with CH₂Cl₂ (10 mL*5). The organic layer was dried over Na₂SO₄, concentrated to give ethyl (E)-4-(tert-butoxycarbonylamino)pent-2-enoate (1 g, crude).

### Step 3. ethyl 4-(tert-butoxycarbonylamino)pentanoate

To a solution of ethyl (E)-4-(tert-butoxycarbonylamino)pent-2-enoate (1 g, 4.11 mmol) in MeOH (50 mL) was added Pd/C (1 g, 10% purity) under H₂ (15 Psi) and stirred at about 25° C. for about 2 hrs. LCMS showed the reaction was complete and the desired mass was detected. The reaction was filtered and concentrated under reduced pressure to give ethyl 4-(tert-butoxycarbonylamino)pentanoate (1.18 g, crude).

### Step 4. tert-butyl N-(-methyl-4-oxo-butyl)carbamate

To a solution of ethyl 4-(tert-butoxycarbonylamino)pentanoate (1 g, 4.08 mmol) in toluene (18.56 mL) was added DIBAL-H (1 M, 6.75 mL) at about −78° C., then stirred at about −78° C. for about 0.75 hrs. TLC (Petroleum ether:Ethyl acetate=5:1, Plate 1) showed (most of) the starting material (R1) was consumed and a new spot was formed. The reaction mixture was quenched by Na₂SO₄·10H₂O. Then stirred for about 2 mins. The reaction was filtered and concentrated under reduced pressure to give tert-butyl N-(1-methyl-4-oxo-butyl)carbamate (1.7 g, crude).

### Step 5. tert-butyl N-(1-methylpent-4-ynyl)carbamate

To a mixture of tert-butyl N-(1-methyl-4-oxo-butyl)carbamate (1.7 g, 8.45 mmol) and K₂CO₃ (2.36 g, 17.06 mmol) in MeOH (10 mL) was added 1-dimethoxyphosphoryl-2-oxo-propane-1-diazonium (1.97 g, 10.20 mmol) at about 0° C., then stirred at about 25° C. for about 12 hrs. TLC (Petroleum ether:Ethyl acetate=5:1) showed the starting material was consumed and a new spot was formed. The reaction was concentrated under reduced pressure to give a residue. The reaction mixture was quenched by NH₄Cl (20 mL), extracted with EtOAc (20 mL*5). The organic layer was dried over Na₂SO₄, concentrated to give the crude product. The residue was purified by prep-TLC (SiO₂, Petroleum ether/Ethyl acetate=5:1) to give tert-butyl N-(1-methylpent-4-ynyl)carbamate (400 mg, crude).

¹H NMR (400 MHz, CHLOROFORM-d) δ:4.52-4.35 (m, 1H), 3.79-3.65 (m, 1H), 2.25-2.17 (m, 2H), 1.93 (t, J=2.6 Hz, 1H), 1.63 (q, J=7.0 Hz, 2H), 1.41 (s, 9H), 1.15-1.09 (m, 3H)

### Step 6. tert-butyl N-allyl-N-(1-methylpent-4-ynyl)carbamate

To the mixture of 3-iodoprop-1-ene (375.13 mg, 2.23 mmol, 204.21 μL) and tert-butyl N-(1-methylpent-4-ynyl)carbamate (400 mg, 2.03 mmol) in DMF (4 mL) was added NaH (166.45 mg, 4.16 mmol, 60% purity) at about 0° C., then stirred at about 25° C. for about 2 hrs under N2. TLC (Petroleum ether:Ethyl acetate=5:1) showed the starting material was consumed and a new spot was formed. The reaction mixture was quenched by NH₄Cl (20 mL), extracted with EtOAc (20 mL*5). The organic layer was dried over Na₂SO₄, concentrated to give the crude product. The residue was purified by prep-TLC (SiO₂, Petroleum ether/Ethyl acetate=5:1) to give tert-butyl N-allyl-N-(1-methylpent-4-ynyl)carbamate (150 mg, crude).

¹H NMR (400 MHz, CHLOROFORM-d) δ:5.91-5.66 (m, 1H), 4.22-4.06 (m, 1H), 3.81-3.57 (m, 2H), 2.16-2.10 (m, 2H), 1.93-1.91 (m, 1H), 1.85-1.74 (m, 1H), 1.66-1.54 (m, 1H), 1.46-1.40 (m, 9H), 1.15-1.10 (m, 3H)

### Step 7. tert-butyl N-allyl-N-[1-methyl-4-(4,4,5,5-tetramethyl-1,3,2-dioxaborolan-2-yl)pent-4-enyl]carbamate

A mixture of LiCl (32.05 mg, 755.94 μmol, 15.50 μL) and CuCl (74.84 mg, 755.94 mol) in DMF (4 mL) was stirred at about 25° C. for about 1 hr under N2, then to the solution was added tert-butyl N-allyl-N-(1-methylpent-4-ynyl)carbamate (150 mg, 632.01 μmol), 4,4,5,5-tetramethyl-2-(4,4,5,5-tetramethyl-1,3,2-dioxaborolan-2-yl)-1,3,2-dioxaborolane (191.96 mg, 755.94 μmol) and KOAc (213.25 mg, 755.94 μmol) and stirred at about 25° C. for about 12 hrs under N2. TLC (Petroleum ether:Ethyl acetate=5:1, Plate 1) showed (most of) the starting material (R1) was consumed and a new spot was formed. The reaction was filtered and added to NH₄Cl (50 mL), extracted with EtOAc (50 mL*5). The organic layer was dried over Na₂SO₄, concentrated to give the crude product. The residue was purified by prep-TLC (SiO₂, Petroleum ether/Ethyl acetate=5:1) to give tert-butyl N-allyl-N-[1-methyl-4-(4,4,5,5-tetramethyl-1,3,2-dioxaborolan-2-yl)pent-4-enyl]carbamate (100 mg, crude).

¹H NMR (400 MHz, CHLOROFORM-d) δ:5.77 (br s, 1H), 5.75-5.71 (m, 1H), 5.61-5.55 (m, 1H), 5.20-4.95 (m, 2H), 4.31-4.13 (m, 1H), 3.76-3.68 (m, 2H), 1.44 (br s, 9H), 1.27-1.22 (m, 12H), 1.16-1.07 (m, 3H)

### Step 8. tert-butyl N-allyl-N-[1-methyl-4-(4,4,5,5-tetramethyl-1,3,2-dioxaborolan-2-yl)pent-4-enyl]carbamate

A solution of tert-butyl N-allyl-N-[1-methyl-4-(4,4,5,5-tetramethyl-1,3,2-dioxaborolan-2-yl)pent-4-enyl]carbamate (638 mg, 1.75 mmol) in DCM (10 mL) was added Hoveyda-Grubbs Catalyst 2nd Generation (52.53 mg, 83.83 mol), then stirred at about 30° C. for about 12 hrs. TLC (Petroleum ether:Ethyl acetate=5:1, Plate 1) showed (most of) the starting material (R1) was consumed and a new spot was formed. The reaction was concentrated under reduced pressure to give a residue. The residue was purified by prep-TLC (SiO₂, Petroleum ether/Ethyl acetate=5:1) to give tert-butyl 2-methyl-5-(4,4,5,5-tetramethyl-1,3,2-dioxaborolan-2-yl)-2,3,4,7-tetrahydroazepine-1-carboxylate (472 mg, crude).

### Step 9. tert-butyl 5-(5-amino-6-fluoro-2,3-dihydrobenzofuran-7-yl)-2-methyl-2,3,4,7-tetrahydroazepine-1-carboxylate

To a mixture of 7-bromo-6-fluoro-2,3-dihydrobenzofuran-5-amine (28.67 mg, 123.54 mol) in H₂O (0.5 mL) and dioxane (5 mL) was added tripotassium;carbonate (51.22 mg, 370.63 μmol), cyclopentyl(diphenyl)phosphane;dichloromethane;dichloropalladium;iron (20.18 mg, 24.71 μmol), then the mixture was stirred at about 100° C. for about 12 hrs under N2. LCMS showed the starting material remained and the desired mass was detected. The reaction was filtered and concentrated under reduced pressure to give a residue. The residue was purified by prep-TLC (SiO₂, Ethyl acetate/Petroleum ether=5:1) to give tert-butyl 5-(5-amino-6-fluoro-2,3-dihydrobenzofuran-7-yl)-2-methyl-2,3,4,7-tetrahydroazepine-1-carboxylate (100 mg, crude).

### Step 10. tert-butyl 5-[6-fluoro-5-[[4-methyl-6-(methylamino)pyrimidin-2-yl]amino]-2,3-dihydrobenzofuran-7-yl]-2-methyl-2,3,4,7-tetrahydroazepine-1-carboxylate

A mixture of 2-chloro-N,6-dimethyl-pyrimidin-4-amine (21.74 mg, 137.95 μmol) and 2-chloro-N,6-dimethyl-pyrimidin-4-amine (21.74 mg, 137.95 μmol) in i-PrOH (3 mL) was stirred at about 130° C. for about 1 hr. LCMS showed the starting material remained and the desired mass was detected. The two reactions were work up together. The reaction mixture was concentrated to give tert-butyl 5-[6-fluoro-5-[[4-methyl-6-(methylamino)pyrimidin-2-yl]amino]-2,3-dihydrobenzofuran-7-yl]-2-methyl-2,3,4,7-tetrahydroazepine-1-carboxylate (260 mg, crude).

### Step 11. N2-[6-fluoro-7-(2-methyl-2, 3, 4, 7-tetrahydro-1H-azepin-5-yl]-2,3-dihydrobenzofuran-5-yl]-N4,6-dimethyl-pyrimidine-2,4-diamine

To a solution of tert-butyl 5-[6-fluoro-5-[[4-methyl-6-(methylamino)pyrimidin-2-yl]amino]-2,3-dihydrobenzofuran-7-yl]-2-methyl-2,3,4,7-tetrahydroazepine-1-carboxylate (250 mg, 516.98 μmol) in DCM (5 mL) was added ZnBr₂ (582.12 mg, 2.58 mmol), stirred at about 25° C. for about 2 hrs. LCMS showed the reaction was complete and the desired mass was detected. The 2 batches were concentrated under reduced pressure to give a residue. The crude product was purified by prep-HPLC(HPLC; TFA condition:column:3_Phenomenex Luna C18 75*30 mm*3 um; mobile phase:water(TFA)-ACN; B %: 1%-30%,3 min) to give N2-[6-fluoro-7-(2-methyl-2,3,4,7-tetrahydro-1H-azepin-5-yl)-2,3-dihydrobenzofuran-5-yl]-N4,6-dimethyl-pyrimidine-2,4-diamine (14.7 mg, 29.61 μmol, 2.86% yield, TFA).

¹H NMR (400 MHz, METHANOL-d₄) δ: 7.42-7.34 (m, 1H), 6.10-6.03 (m, 1H), 5.98 (s, 1H), 4.66 (dt, J=4.4, 8.8 Hz, 2H), 3.90 (d, J=7.0 Hz, 2H), 3.65-3.51 (m, 1H), 3.28-3.22 (m, 2H), 2.95-2.86 (m, 3H), 2.86-2.78 (m, 1H), 2.71-2.59 (m, 1H), 2.39-2.26 (m, 3H), 2.13-2.05 (m, 1H), 1.92-1.81 (m, 1H), 1.39 (d, J=6.6 Hz, 3H)

### EXAMPLE 47. Synthesis of Compound 183 and Compound 184

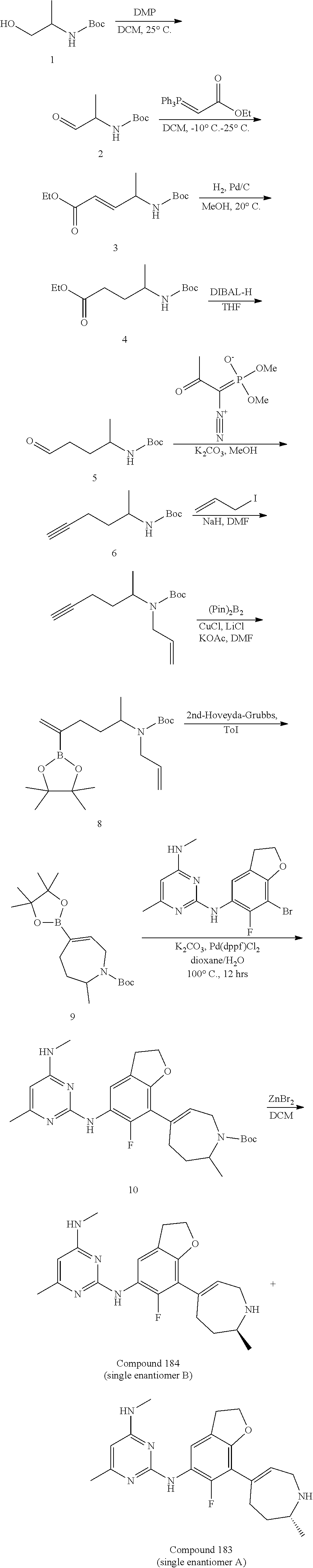

### Step 1. tert-butyl N-(1-methyl-2-oxo-ethyl)carbamate

To a solution of (1,1-diacetoxy-3-oxo-1,2-benziodoxol-1-yl) acetate (49.20 g, 116.00 mmol) in DCM (50 mL) was added tert-butyl N-(2-hydroxy-1-methyl-ethyl)carbamate (20 g, 114.14 mmol) at about 0° C., then stirring at about 25° C. for about 12 hrs. TLC (Petroleum ether:Ethyl acetate=1:1,) showed the starting material was consumed and a new spot was formed. The reaction mixture was quenched by Na₂O₃S₂(100 mL), extracted with CH₂Cl₂ (100 mL*3). The organic layer was dried over Na₂SO₄, concentrated to give tert-butyl N-(1-methyl-2-oxo-ethyl)carbamate (37 g, crude).

### Step 2. ethyl (E)-4-(tert-butoxycarbonylamino)pent-2-enoate

To a solution of tert-butyl N-(1-methyl-2-oxo-ethyl)carbamate (37 g, 213.61 mmol) in DCM (500 mL) was added ethyl 2-(triphenyl-phosphanylidene)acetate (74.42 g, 213.61 mmol) at about 0° C., then stirred at about 25° C. for about 2 hrs. LCMS showed the reaction was complete and the desired mass was detected. The reaction mixture was added to water (200 mL), extracted with DCM (200 mL*3). The organic layer was dried over Na₂SO₄, concentrated to give ethyl (E)-4-(tert-butoxycarbonylamino)pent-2-enoate (21 g, crude).

### Step 3. ethyl 4-(tert-butoxycarbonylamino)pentanoate

To a solution of ethyl (E)-4-(tert-butoxycarbonylamino)pent-2-enoate (21 g, 86.31 mmol) in MeOH (300 mL) was added Pd/C (10 g, 10% purity) under H₂ (15 Psi) and stirred at about 25° C. for about 2 hrs. LCMS showed the reaction was complete and the desired mass was detected. The reaction was filtered and concentrated under reduced pressure to give ethyl 4-(tertbutoxycarbonylamino)pentanoate (22 g, crude).

### Step 4. tert-butyl N-(-methyl-4-oxo-butyl)carbamate

To a solution of ethyl 4-(tert-butoxycarbonylamino)pentanoate (13 g, 52.99 mmol) in toluene (260.00 mL) was added DIBAL-H (1 M, 87.75 mL) at about −78° C., then stirred at about −78° C. for about 0.75 hrs. TLC (Petroleum ether:Ethyl acetate=5:1) showed the starting material was consumed and a new spot was formed. The reaction mixture was quenched by NaSO₄·10H₂O. Then stirred for about 20 mins. The reaction was filtered and concentrated under reduced pressure to give tert-butyl N-(1-methyl-4-oxo-butyl)carbamate (21 g, crude).

### Step 5. tert-butyl N-(-methylpent-4-ynyl)carbamate

To a mixture of tert-butyl N-(1-methyl-4-oxo-butyl)carbamate (21 g, 104.34 mmol) and K₂CO₃ (29.13 g, 210.77 mmol) in MeOH (210 mL) was added 1-dimethoxyphosphoryl-2-oxo-propane-1-diazonium (24.33 g, 125.98 mmol) at about 0° C., then stirred at about 25° C. for about 12 hrs. TLC (Petroleum ether:Ethyl acetate=5:1) showed the starting material was consumed and a new spot was formed. The reaction was concentrated under reduced pressure to give a residue. The reaction mixture was quenched by NH₄Cl (50 mL), extracted with EtOAc (100 mL*5). The organic layer was dried over Na₂SO₄, concentrated to give the crude product.

The residue was purified by column chromatography (SiO₂, Petroleum ether/Ethyl acetate=1/0 to 20:1) to give tert-butyl N-(1-methylpent-4-ynyl)carbamate (4.6 g, crude).

¹H NMR (400 MHz, CHLOROFORM-d) δ:3.78-3.65 (m, 1H), 2.26-2.18 (m, 2H), 1.64 (q, J=7.1 Hz, 2H), 1.42 (s, 9H), 1.13 (d, J=6.7 Hz, 3H)

### Step 6. tert-butyl N-allyl-N-(1-methylpent-4-ynyl)carbamate

To the mixture of 3-iodoprop-1-ene (7.50 g, 44.67 mmol, 4.08 mL) and tert-butyl N-(1-methylpent-4-ynyl)carbamate (8 g, 40.55 mmol) in DMF (100 mL) was added NaH (3.33 g, 83.23 mmol, 60% purity), then stirred at about 25° C. for about 2 hrs under N2. TLC (Petroleum ether:Ethyl acetate=5:1) showed the starting material was consumed and a new spot was formed. The reaction mixture was quenched by NH₄Cl (20 mL), extracted with EtOAc (20 mL*5). The residue was purified by column chromatography (SiO₂, Petroleum ether/Ethyl acetate=20/1 to 0/1) to give tert-butyl N-allyl-N-(1-methylpent-4-ynyl)carbamate (5.9 g, crude).

### Step 7. tert-butyl N-allyl-N-[I-methyl-4-(4,4,5,5-tetramethyl-1,3,2-dioxaborolan-2-yl)pent-4-enyl]carbamate

A mixture of LiCl (1.26 g, 29.73 mmol, 609.54 μL) and CuCl (2.94 g, 29.73 mmol) in DMF (100 mL) was stirred at about 25° C. for about 1 hr under N2, then to the solution was added tert-butyl N-allyl-N-(1-methylpent-4-ynyl)carbamate (5.9 g, 24.86 mmol), 4,4,5,5-tetramethyl-2-(4,4,5,5-tetramethyl-1,3,2-dioxaborolan-2-yl)-1,3,2-dioxaborolane (7.55 g, 29.73 mmol) and KOAc (8.39 g, 29.73 mmol) in DMF (50.00 mL) and stirred at about 25° C. for about 12 hrs under N2. TLC (Petroleum ether:Ethyl acetate=10:1) showed the starting material was consumed and a new spot was formed. LCMS showed the reaction was complete and the desired mass was detected. The reaction was filtered and added to NH₄Cl (20 mL), extracted with EtOAc (20 mL*5). The organic layer was dried over Na₂SO₄, concentrated to give the crude product. The residue was purified by column chromatography (SiO₂, Petroleum ether/Ethyl acetate=50/1 to 10/1) to give tert-butyl N-allyl-N-[1-methyl-4-(4,4,5,5-tetramethyl-1,3,2-dioxaborolan-2-yl)pent-4-enyl]carbamate (6.4 g, crude).

### Step 8. tert-butyl N-allyl-N-f]-methyl-4-(4,4,5,5-tetramethyl-1,3,2-dioxaborolan-2-yl)pent-4-enyl]carbamate

To a solution of tert-butyl N-allyl-N-[1-methyl-4-(4,4,5,5-tetramethyl-1,3,2-dioxaborolan-2-yl)pent-4-enyl]carbamate (3.1 g, 8.49 mmol) in DCM (300 mL) was added Hoveyda-Grubbs Catalyst 2nd Generation (255.24 mg, 407.32 μmol), then stirred at about 30° C. for about 12 hrs under N2. TLC (Petroleum ether:Ethyl acetate=5:1, Plate 1) showed (most of) the starting material (R1) was consumed and a new spot was formed. LCMS showed the reaction was complete and the desired mass was detected. The reaction was concentrated under reduced pressure to give a residue. The residue was purified by column chromatography (SiO₂, Petroleum ether/Ethyl acetate=32/1 to 0/1) to give tert-butyl 2-methyl-5-(4,4,5,5-tetramethyl-1,3,2-dioxaborolan-2-yl)-2,3,4,7-tetrahydroazepine-1-carboxylate (2.07 g, crude).

### Step 9. tert-butyl 5-(5-amino-6-fluoro-2,3-dihydrobenzofuran-7-yl)-2-methyl-2,3,4,7-tetrahydroazepine-1-carboxylate

To a mixture of N2-(7-bromo-6-fluoro-2,3-dihydrobenzofuran-5-yl)-N4,6-dimethyl-pyrimidine-2,4-diamine (1 g, 2.83 mmol) and K₂CO₃ (1.17 g, 8.49 mmol) was added cyclopentyl(diphenyl)phosphane;dichloropalladium;iron (414.34 mg, 566.27 μmol) in dioxane (10 mL) and H₂O (0.1 mL), then stirred at about 100° C. for about 12 hrs under N2. LCMS showed the starting material remained and the desired mass was detected. The reaction was concentrated under reduced pressure to give tert-butyl 5-[6-fluoro-5-[[4-methyl-6-(methylamino)pyrimidin-2-yl]amino]-2,3-dihydrobenzofuran-7-yl]-2-methyl-2,3,4,7-tetrahydroazepine-1-carboxylate (1.3 g, crude).

### Step 10. N2-[6-fluoro-7-[rel-(2S)-2-methyl-2,3,4,7-tetrahydro-1H-azepin-5-yl]-2,3-dihydrobenzofuran-5-yl]-N4,6-dimethyl-pyrimidine-2,4-diamine and N2-[6-fluoro-7-[rel-(2R)-2-methyl-2,3,4,7-tetrahydro-1H-azepin-5-yl]-2,3-dihydrobenzofuran-5-yl]-N4,6-dimethyl-pyrimidine-2,4-diamine

To a solution of tert-butyl 5-[6-fluoro-5-[[4-methyl-6-(methylamino)pyrimidin-2-yl]amino]-2,3-dihydrobenzofuran-7-yl]-2-methyl-2,3,4,7-tetrahydroazepine-1-carboxylate (1.3 g, 2.69 mmol) in DCM (20 mL) was added ZnBr₂ (3.03 g, 13.44 mmol) and then the reaction mixture was stirred at about 25° C. for about 2 hrs. LCMS showed the reaction was complete and the desired mass was detected. The reaction was filtered and concentrated under reduced pressure to give a residue. The crude product was purified by prep-HPLC(TFA condition:column:3_Phenomenex Luna C18 75*30 mm*3 um; mobile phase:water(TFA)-ACN; B %: 1%-25%, 8 min) to give a mixture of Compound 183 and Compound 184.

The mixture was separated by SFC (column: Lux Cellulose-2, 50×4.6 mm I.D.; mobile phase: A: CO2 B:MeOH(0.1% IPAm, v/v; Acq Method: C2_MeOH_IPAm_50_4_35) to give N2-[6-fluoro-7-[rel-(2S)-2-methyl-2,3,4,7-tetrahydro-1H-azepin-5-yl]-2,3-dihydrobenzofuran-5-yl]-N4,6-dimethyl-pyrimidine-2,4-diamine (129.90 mg, 338.76 μmol) (de %=100%, Rt=1.228 min) and N2-[6-fluoro-7-[rel-(2R)-2-methyl-2,3,4,7-tetrahydro-1H-azepin-5-yl]-2,3-dihydrobenzofuran-5-yl]-N4,6-dimethyl-pyrimidine-2,4-diamine (177.70 mg, 463.41 μmol) (de %=100%, Rt=1.893 min).

¹H NMR (400 MHz, METHANOL-d₄) δ: 7.77 (s, 1H), 6.03-5.94 (m, 1H), 5.81-5.74 (m, 1H), 4.61-4.50 (m, 2H), 3.54-3.40 (m, 2H), 3.23-3.14 (m, 2H), 3.10-2.99 (m, 1H), 2.90-2.81 (m, 3H), 2.66-2.47 (m, 2H), 2.19-2.12 (m, 3H), 1.96-1.83 (m, 1H), 1.68-1.52 (m, 1H), 1.24-1.09 (m, 3H)

¹H NMR (400 MHz, METHANOL-d₄) δ: 7.39 (br d, J=7.5 Hz, 1H), 6.06 (br t, J=6.2 Hz, 1H), 5.98 (s, 1H), 4.66 (dt, J=3.4, 8.7 Hz, 2H), 3.90 (d, J=6.5 Hz, 2H), 3.64-3.52 (m, 1H), 3.29-3.21 (m, 2H), 2.89 (s, 3H), 2.85 (br s, 2H), 2.28 (s, 2H), 1.39 (d, J=6.6 Hz, 3H)

### EXAMPLE 48. Synthesis of Compound 188

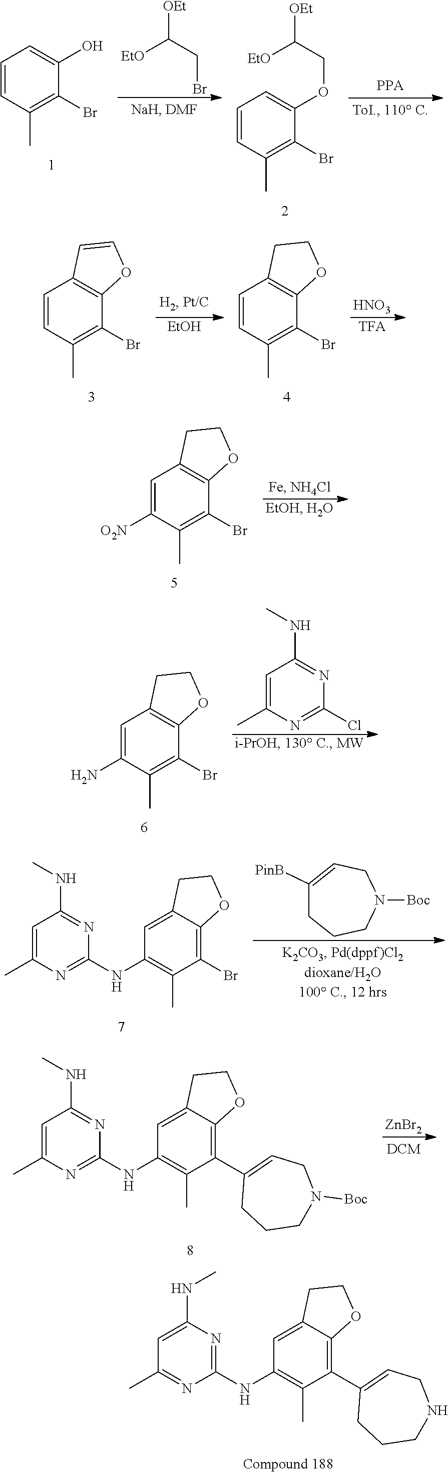

### Step 1. 2-bromo-1-(2,2-diethoxyethoxy)-3-methyl-benzene

To a solution of 2-bromo-3-methyl-phenol (1 g, 5.35 mmol) in DMF (10 mL) was added NaH (320.77 mg, 8.02 mmol, 156.93 μL, 60% purity) at about 0° C. It was stirred at about 0° C. for about 0.5 h. Then to it was added 2-bromo-1,1-diethoxy-ethane (1.26 g, 6.42 mmol) at about 0° C. It was stirred at about 80° C. for about 12 h. The reaction was poured into sat. NH₄Cl (50 mL), then extracted with ethyl acetate (100 mL*3). The combined organic layers were washed with brine (50 mL) and dried over anhydrous sodium sulfate. It was filtered and concentrated under reduced pressure to give a residue. The residue was purified by flash chromatography (silica gel, petroleum ether/ethyl acetate=1/0 to 100/1) to give 2-bromo-1-(2,2-diethoxyethoxy)-3-methyl-benzene (0.9 g, 2.97 mmol).

¹H NMR (400 MHz, CHLOROFORM-d) δ:1.19 (t, J=7.07 Hz, 6H) 2.34 (s, 3H) 3.60-3.68 (m, 2H) 3.74 (dq, J=9.33, 7.10 Hz, 2H) 3.97 (d, J=5.25 Hz, 2H) 4.82 (t, J=5.19 Hz, 1H) 6.66 (d, J=8.13 Hz, 1H) 6.79 (d, J=7.75 Hz, 1H) 7.06 (t, J=7.94 Hz, 1H)

### Step 2. 7-bromo-6-methyl-benzofuran

To a solution of 2-bromo-1-(2,2-diethoxyethoxy)-3-methyl-benzene (0.9 g, 2.97 mmol) in toluene (18 mL) was added PPA (0.9 g). It was stirred at about 110° C. for about 12 h. It was concentrated under reduced pressure to give a residue. The residue was purified by flash chromatography (silica gel, petroleum ether/ethyl acetate=1/0 to 100/1) to give 7-bromo-6-methyl-benzofuran (360 mg, 1.71 mmol).

¹H NMR (400 MHz, CHLOROFORM-d) δ ppm 2.45 (s, 3H) 6.72 (d, J=2.00 Hz, 1H) 7.05 (d, J=7.88 Hz, 1H) 7.33 (d, J=7.88 Hz, 1H) 7.55 (d, J=2.00 Hz, 1H)

### Step 3. 7-bromo-6-methyl-2,3-dihydrobenzofuran

To a solution of 7-bromo-6-methyl-benzofuran (340 mg, 1.61 mmol) in EtOH (35 mL) was added Pt/C (340 mg, 5% purity). The suspension was degassed under vacuum and purged with H₂ (15 PSI) several times. It was stirred at about 20° C. for about 12 h under H₂ (15 PSI). The reaction mixture was filtered and concentrated under reduced pressure to give 7-bromo-6-methyl-2,3-dihydrobenzofuran (290 mg, crude).

¹H NMR (400 MHz, CHLOROFORM-d) δ:2.29 (s, 3H) 3.22 (br t, J=8.63 Hz, 2H) 4.59 (t, J=8.63 Hz, 2H) 6.66 (d, J=7.38 Hz, 1H) 6.92 (br d, J=7.38 Hz, 1H)

### Step 4. 7-bromo-6-methyl-5-nitro-2,3-dihydrobenzofuran

To a solution of 7-bromo-6-methyl-2,3-dihydrobenzofuran (40 mg, 187.73 μmol) in TFA (0.8 mL) was added HNO₃ (52.19 mg, 563.19 mol, 68% purity) at about 10° C. It was stirred at about 20° C. for about 1 h. It was concentrated to give 7-bromo-6-methyl-5-nitro-2,3-dihydrobenzofuran (45 mg, crude).

### Step 5. 7-bromo-6-methyl-2,3-dihydrobenzofuran-5-amine

To a solution of 7-bromo-6-methyl-5-nitro-2,3-dihydrobenzofuran (45 mg, 174.37 μmol) in H₂O (0.3 mL) and EtOH (1 mL) was added NH₄Cl (137.17 mg, 1.74 mmol, 68% purity), Fe (48.69 mg, 871.86 μmol). The reaction mixture was stirred at about 80° C. for about 1 h. It was filtered and concentrated under reduced pressure, then added into water (5 mL), then extracted with ethyl acetate (10 mL*3). The combined organic layers were washed with brine (5 mL) and dried over anhydrous sodium sulfate and then filtered, the filtrate was concentrated under reduced pressure to give 7-bromo-6-methyl-2,3-dihydrobenzofuran-5-amine (40 mg, crude).

### Step 6. N2-(7-bromo-6-methyl-2,3-dihydrobenzofuran-5-yl)-N4,6-dimethyl-pyrimidine-2,4-diamine

To a solution of 2-chloro-N,6-dimethyl-pyrimidin-4-amine (27.64 mg, 175.37 mol), 7-bromo-6-methyl-2,3-dihydrobenzofuran-5-amine (40 mg, 175.37 μmol) in i-PrOH (1 mL) was added TFA (2.00 mg, 17.54 mol, 1.35 μL). The reaction mixture was stirred at about 130° C. for about 1 h. It was concentrated under reduced pressure to give N2-(7-bromo-6-methyl-2,3-dihydrobenzofuran-5-yl)-N4,6-dimethyl-pyrimidine-2,4-diamine (60 mg, crude).

### Step 7. tert-butyl 5-[6-methyl-5-[[4-methyl-6-(methylamino)pyrimidin-2-yl]amino]-2,3-dihydrobenzo furan-7-yl]-2,3,4,7-tetrahydroazepine-1-carboxylate

To a solution of tert-butyl 5-(4,4,5,5-tetramethyl-1,3,2-dioxaborolan-2-yl)-2,3,4,7-tetrahydroazepine-1-carboxylate (55.53 mg, 171.81 μmol) and N2-(7-bromo-6-methyl-2,3-dihydrobenzofuran-5-yl)-N4,6-dimethyl-pyrimidine-2,4-diamine (60 mg, 171.81 mol) in H₂O (0.1 mL) and dioxane (1 mL) was added K₂CO₃ (71.23 mg, 515.43 μmol) and Pd(dppf)Cl₂ (12.57 mg, 17.18 μmol), and then the mixture was stirred at about 100° C. for about 12 h under N2. It was concentrated under reduced pressure to give tert-butyl 5-[6-methyl-5-[[4-methyl-6-(methylamino)pyrimidin-2-yl]amino]-2,3-dihydrobenzofuran-7-yl]-2,3,4,7-tetrahydroazepine-1-carboxylate (70 mg, crude).

### Step 8. N4,6-dimethyl-N2-[6-methyl-7-(2,3,4,7-tetrahydro-1H-azepin-5-yl)-2,3-dihydrobenzofuran-5-yl]pyrimidine-2,4-diamine

To a solution of tert-butyl 5-[6-methyl-5-[[4-methyl-6-(methylamino)pyrimidin-2-yl]amino]-2,3-dihydrobenzofuran-7-yl]-2,3,4,7-tetrahydroazepine-1-carboxylate (70 mg, 150.35 mol) in DCM (1 mL) was added ZnBr₂ (169.29 mg, 751.74 mol). The reaction mixture was stirred at about 40° C. for about 12 h. It was concentrated under reduced pressure to give a residue and then purified by prep-HPLC (TFA condition, column: Phenomenex Luna C18 75*30 mm*3 um; mobile phase: [water(0.1% TFA)-ACN]; B %: 5%-35%,7 min) to give N4,6-dimethyl-N2-[6-methyl-7-(2,3,4,7-tetrahydro-1H-azepin-5-yl)-2,3-dihydrobenzofuran-5-yl]pyrimidine-2,4-diamine (21.7 mg, 59.38 μmol) (TFA salt, 96.0310% purity).

¹H NMR (400 MHz, METHANOL-d₄) δ: 2.09-2.15 (m, 2H) 2.18 (s, 3H) 2.26 (s, 3H) 2.67 (br s, 2H) 2.89-2.94 (m, 3H) 3.26 (br t, J=8.69 Hz, 2H) 3.50 (br d, J=1.50 Hz, 2H) 3.93 (br d, J=5.75 Hz, 2H) 4.59-4.64 (m, 2H) 5.75 (t, J=6.25 Hz, 1H) 5.95 (s, 1H) 7.17 (s, 1H)

### EXAMPLE 49. Synthesis of Compound 189

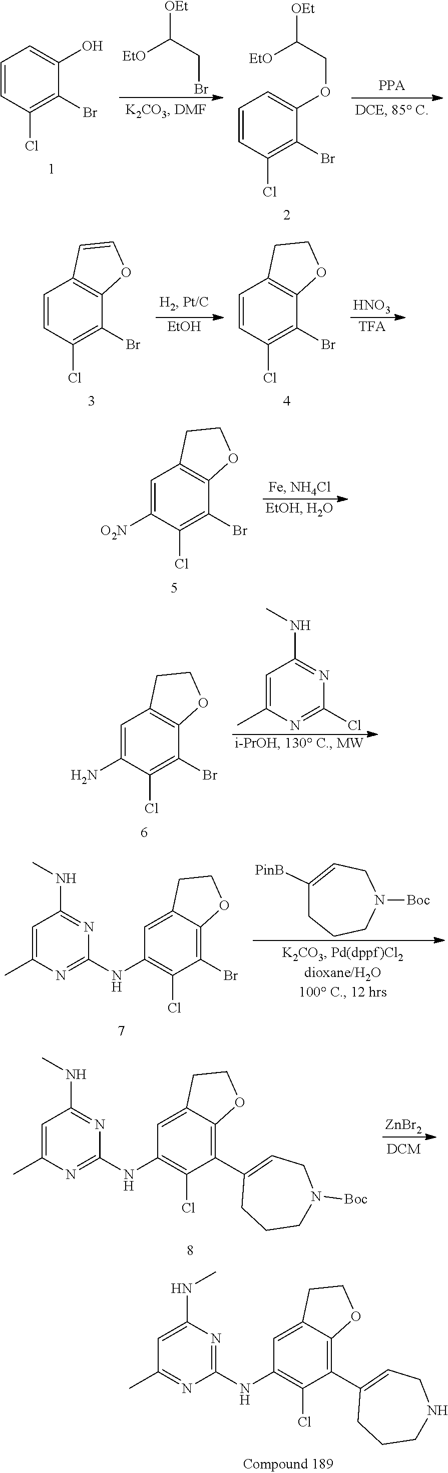

### Step 1. 2-bromo-1-chloro-3-(2,2-diethoxyethoxy)benzene

To a solution of 2-bromo-3-chloro-phenol (5 g, 24.10 mmol) and 2-bromo-1,1-diethoxy-ethane (7.12 g, 36.15 mmol) in DMF (50 mL) was added K₂CO₃ (5.00 g, 36.15 mmol, 2.44 mL). The reaction mixture was stirred at about 120° C. for about 12 h. The mixture was poured into water (70 mL), then extracted with ethyl acetate (70 mL*3). The combined organic layers were washed with brine (50 mL) and dried over anhydrous sodium sulfate and then filtered, the filtrate was concentrated under reduced pressure to give a residue. The residue was purified by flash chromatography (silica gel, petroleum ether/ethyl acetate=1/0 to 5/1) to give 2-bromo-1-chloro-3-(2,2-diethoxyethoxy)benzene (8.5 g, crude).

### Step 2. 7-bromo-6-chloro-benzofuran

To a solution of 2-bromo-1-chloro-3-(2,2-diethoxyethoxy)benzene (7.5 g, 23.18 mmol) in DCE (150 mL) was added PPA (2.87 g, 27.81 mmol). The reaction mixture was stirred at about 85° C. for about 12 h. The reaction mixture was concentrated under reduced pressure to give a residue. The residue was purified by flash chromatography (silica gel, petroleum ether/ethyl acetate=1/0 to 100/1) to give 7-bromo-6-chloro-benzofuran (1.6 g, 6.91 mmol).

¹H NMR (400 MHz, CHLOROFORM-d) δ:6.76 (d, J=2.00 Hz, 1H) 7.27 (d, J=8.25 Hz, 1H) 7.39 (d, J=8.25 Hz, 1H) 7.62 (d, J=2.13 Hz, 1H)

### Step 3. 7-bromo-6-chloro-2,3-dihydrobenzofuran

To a solution of 7-bromo-6-chloro-benzofuran (0.5 g, 2.16 mmol) in EtOH (50 mL) was added Pt/C (0.5 g, 5% purity). The suspension was degassed under vacuum and purged with H₂ several times. It was stirred at about 20° C. for about 12 h under H₂ (15 PSI). It was filtered and concentrated under reduced pressure to give 7-bromo-6-chloro-2,3-dihydrobenzofuran (330 mg, crude).

¹H NMR (400 MHz, CHLOROFORM-d) δ:3.31 (br t, J=8.57 Hz, 2H) 4.72 (br t, J=8.63 Hz, 2H) 6.94-6.99 (m, 1H) 7.04 (br d, J=7.75 Hz, 1H)

### Step 4. 7-bromo-6-chloro-5-nitro-2,3-dihydrobenzofuran

To a solution of 7-bromo-6-chloro-2,3-dihydrobenzofuran (300 mg, 1.28 mmol) in TFA (6 mL) was added dropwise HNO₃ (357.19 mg, 3.85 mmol, 68% purity) at about 10° C. The reaction mixture was stirred at about 20° C. for 1 h. It was concentrated by a stream of N2 to give 7-bromo-6-chloro-5-nitro-2,3-dihydrobenzofuran (350 mg, crude).

### Step 5. 7-bromo-6-chloro-2,3-dihydrobenzofuran-5-amine

To a solution of 7-bromo-6-chloro-5-nitro-2,3-dihydrobenzofuran (350 mg, 1.26 mmol) in H₂O (1 mL) and EtOH (4 mL) was added NH₄Cl (988.64 mg, 12.57 mmol, 68% purity), Fe (350.93 mg, 6.28 mmol). The reaction mixture was stirred at about 80° C. for about 1 h. It was filtered and concentrated under reduced pressure, then added into water (15 mL), then extracted with ethyl acetate (20 mL*3). The combined organic layers were washed with brine (15 mL*3) and dried over anhydrous sodium sulfate. It was filtered and concentrated under reduced pressure to give 7-bromo-6-chloro-2,3-dihydrobenzofuran-5-amine (200 mg, crude).

### Step 6. N2-(7-bromo-6-chloro-2,3-dihydrobenzo furan-5-yl)-N4,6-dimethyl-pyrimidine-2,4-diamine

To a solution of 2-chloro-N,6-dimethyl-pyrimidin-4-amine (126.84 mg, 804.82 mol), 7-bromo-6-chloro-2,3-dihydrobenzofuran-5-amine (200 mg, 804.82 μmol) in i-PrOH (2 mL) was added TFA (9.18 mg, 80.48 mol, 6.20 μL). The reaction mixture was stirred at about 130° C. for about 1 h. After the reaction was complete, it was filtered and the filter cake was dried in vacuum to give N2-(7-bromo-6-chloro-2,3-dihydrobenzofuran-5-yl)-N4,6-dimethyl-pyrimidine-2,4-diamine (190 mg, crude).

### Step 7: tert-butyl 5-[6-chloro-5-[[4-methyl-6-(methylamino)pyrimidin-2-yl-amino-2,3-dihydrobenzofuran-7-yl]-2,3,4,7-tetrahydroazepine-1-carboxylate

To a solution of tert-butyl 5-(4,4,5,5-tetramethyl-1,3,2-dioxaborolan-2-yl)-2,3,4,7-tetrahydroazepine-1-carboxylate (43.72 mg, 135.27 μmol) and N2-(7-bromo-6-chloro-2,3-dihydrobenzofuran-5-yl)-N4,6-dimethyl-pyrimidine-2,4-diamine (50 mg, 135.27 μmol) in H₂O (0.1 mL) and dioxane (1 mL) was added K₂CO₃ (56.08 mg, 405.80 μmol) and Pd(dppf)Cl₂ (9.90 mg, 13.53 μmol), and then the mixture was stirred at about 100° C. for about 3 h under N2. The reaction mixture was filtered and the filtrate was concentrated under reduced pressure to give tert-butyl 5-[6-chloro-5-[[4-methyl-6-(methylamino)pyrimidin-2-yl]amino]-2,3-dihydrobenzofuran-7-yl]-2,3,4,7-tetrahydroazepine-1-carboxylate (65 mg, crude).

### Step 8. N2-[6-chloro-7-(2,3,4,7-tetrahydro-1H-azepin-5-yl)-2,3dihydrobenzofuran-5-yl]-N4,6-dimethyl-pyrimidine-2,4-diamine

To a solution of tert-butyl 5-[6-chloro-5-[[4-methyl-6-(methylamino)pyrimidin-2-yl]amino]-2,3-dihydrobenzofuran-7-yl]-2,3,4,7-tetrahydroazepine-1-carboxylate (65 mg, 133.74 μmol) in DCM (1 mL) was added ZnBr₂ (150.59 mg, 668.72 mol). The reaction mixture was stirred at about 40° C. for about 12 h. It was concentrated under reduced pressure to give a residue. The residue was purified by prep-HPLC (TFA condition, column: Phenomenex Luna C18 75*30 mm*3 um; mobile phase: [water(0.1% TFA)-ACN]; B %: 1%-30%,7 min) to give N2-[6-chloro-7-(2,3,4,7-tetrahydro-1H-azepin-5-yl)-2,3-dihydrobenzofuran-5-yl]-N4,6-dimethyl-pyrimidine-2,4-diamine (26.2 mg, 67.90 μmol) (99.624% purity, TFA salt).

¹H NMR (400 MHz, METHANOL-d₄) δ:2.13 (br d, J=3.88 Hz, 2H) 2.27 (s, 3H) 2.67 (br d, J=4.50 Hz, 2H) 2.87-2.93 (m, 3H) 3.25-3.30 (m, 2H) 3.46-3.52 (m, 2H) 3.92 (br d, J=6.13 Hz, 2H) 4.65 (br t, J=8.69 Hz, 2H) 5.87 (br t, J=6.13 Hz, 1H) 5.98 (s, 1H) 7.44 (s, 1H)

### EXAMPLE 50. Synthesis of Compound 193

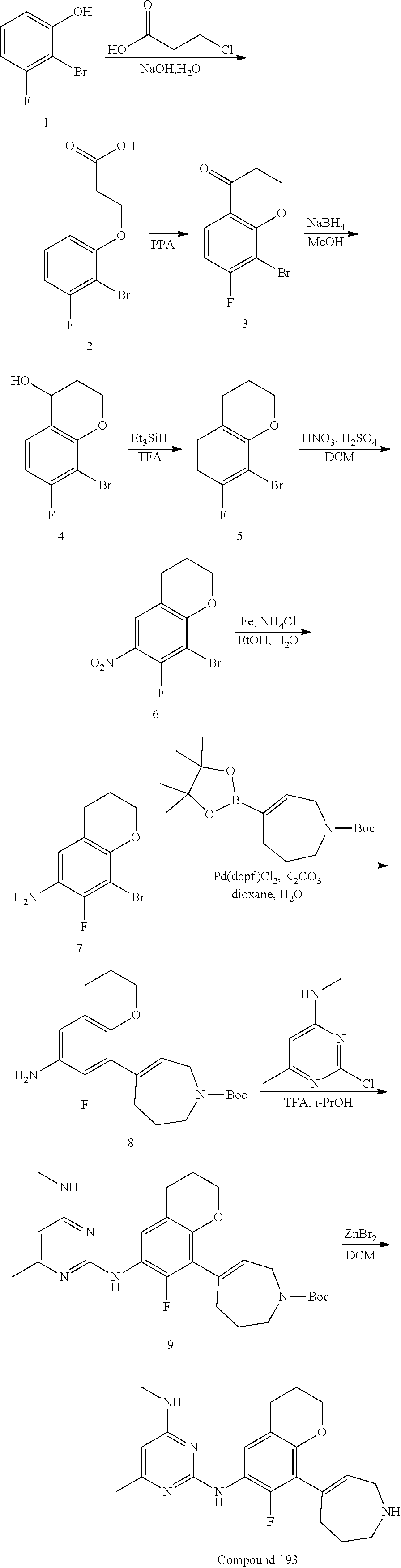

### Step 1. 3-(2-bromo-3-fluoro-phenoxy)propanoic acid

A mixture of 2-bromo-3-fluoro-phenol (10 g, 52.36 mmol) and 3-chloropropanoic acid (6.82 g, 62.83 mmol, 5.37 mL) was added to a flask, then a solution of NaOH (5.03 g, 125.66 mmol, 2.36 mL) in H₂O (25 mL) was added dropwise at 20° C., then the mixture was stirred at about 100° C. for about 12 h. TLC (petroleum ether:ethyl acetate=3:1, Rf=0.5) indicated 2-bromo-3-fluoro-phenol remained, and one major new spot was detected. To the reaction was added 3N HCl to pH=4, then extracted with ethyl acetate (100 mL*3) and dried over anhydrous sodium sulfate, filtered and concentrated under reduced pressure to give a residue. The residue was purified by flash chromatography (silica gel, petroleum ether/ethyl acetate=1/0 to 1/1) to give 3-(2-bromo-3-fluoro-phenoxy)propanoic acid (4.5 g, 17.11 mmol).

¹H NMR (400 MHz, METHANOL-d₄) δ: 2.82 (t, J=6.07 Hz, 2H) 4.32 (t, J=6.07 Hz, 2H) 6.81 (td, J=8.35, 1.19 Hz, 1H) 6.88 (dt, J=8.38, 1.13 Hz, 1H) 7.30 (td, J=8.41, 6.44 Hz, 1H)

### Step 2. 8-bromo-7-fluoro-chroman-4-one

A solution of 3-(2-bromo-3-fluoro-phenoxy)propanoic acid (4.5 g, 17.11 mmol) and PPA (50 mL) was stirred at about 100° C. for about 2 h. TLC (petroleum ether:ethyl acetate=3:1, Rf=0.6) indicated 3-(2-bromo-3-fluoro-phenoxy)propanoic acid was consumed completely, and one major new spot was detected. LCMS showed starting material was consumed completely and mass of the desired compound. The reaction was added into water (500 mL), then extracted with ethyl acetate (3*500 mL) and dried over anhydrous sodium sulfate, filtered and concentrated under reduced pressure to give a residue. The residue was purified by flash chromatography (silica gel, petroleum ether/ethyl acetate=1/0 to 10/1) to give 8-bromo-7-fluoro-chroman-4-one (4.2 g, crude).

¹H NMR (400 MHz, METHANOL-d₄) δ: 2.85 (t, J=6.47 Hz, 2H) 4.69 (t, J=6.47 Hz, 2H) 6.93 (t, J=8.55 Hz, 1H) 7.89 (dd, J=8.55, 6.58 Hz, 1H)

### Step 3. 8-bromo-7-fluoro-chroman-4-ol

To a solution of 8-bromo-7-fluoro-chroman-4-one (4.56 g, 18.61 mmol) in MeOH (45 mL) was added NaBH₄ (1.06 g, 27.91 mmol) at about 0° C., then the mixture was stirred at about 20° C. for about 1 h. TLC (petroleum ether:ethyl acetate=1:1, Rf=0.5) indicated 8-bromo-7-fluoro-chroman-4-one was consumed completely, and one major new spot was detected. LCMS showed starting material was consumed completely and mass of the desired compound. The reaction was concentrated under reduced pressure to give 8-bromo-7-fluoro-chroman-4-ol (4.6 g, crude).

¹H NMR (400 MHz, METHANOL-d₄) δ:1.97-2.05 (m, 1H) 2.06-2.14 (m, 1H) 4.29-4.42 (m, 2H) 4.74 (t, J=4.25 Hz, 1H) 6.77 (t, J=8.44 Hz, 1H) 7.32 (dd, J=8.57, 6.32 Hz, 1H)

### Step 4. 8-bromo-7-fluoro-chromane

To a solution of 8-bromo-7-fluoro-chroman-4-ol (4.6 g, 18.62 mmol) in TFA (50 mL) was added triethylsilane (4.33 g, 37.24 mmol, 5.95 mL), then the mixture was stirred at about 60° C. for about 12 h. TLC (petroleum ether:ethyl acetate=10:1, Rf=0.5) indicated starting material was consumed completely, and one major new spot was detected. The reaction was added into water (50 mL), then extracted with ethyl acetate (3*50 mL). The combined organic layers were washed with brine (50 mL) and dried over anhydrous sodium sulfate, filtered and concentrated under reduced pressure to give a residue. The residue was purified by flash chromatography (silica gel, petroleum ether/ethyl acetate=1/0) to give 8-bromo-7-fluoro-chromane (3.65 g, 15.80 mmol).

¹H NMR (400 MHz, METHANOL-d₄) δ: 1.93-2.03 (m, 2H) 2.79 (t, J=6.44 Hz, 2H) 4.22-4.34 (m, 2H) 6.66 (t, J=8.44 Hz, 1H) 6.95-7.09 (m, 1H)

### Step 5. 8-bromo-7-fluoro-6-nitro-chromane

To a solution of 8-bromo-7-fluoro-chromane (3.35 g, 14.50 mmol) in DCM (50 mL) was added a mixture of nitric acid (959.26 mg, 15.22 mmol, 648.15 μL) and sulfuric acid (5.80 g, 57.99 mmol, 3.15 mL, 98% purity) at about −5° C., then the mixture was stirred at about 20° C. for about 1 h. TLC (petroleum ether:ethyl acetate=3:1, Rf=0.6) indicated starting material was consumed completely, and one major new spot was detected. The reaction was added into water (50 mL) at about 0° C., then extracted with dichloromethane (100 mL*3). The combined organic layers were washed with saturated sodium bicarbonate solution (3*100 mL) and dried over anhydrous sodium sulfate, filtered and concentrated under reduced pressure to give 8-bromo-7-fluoro-6-nitro-chromane (3.43 g, crude).

¹H NMR (400 MHz, METHANOL-d₄) δ: 2.02-2.09 (m, 2H) 2.88 (t, J=6.47 Hz, 2H) 4.39-4.45 (m, 2H) 7.95 (d, J=8.33 Hz, 1H)

### Step 6. 8-bromo-7-fluoro-chroman-6-amine

To a solution of 8-bromo-7-fluoro-6-nitro-chromane (3.43 g, 12.42 mmol), Fe (3.47 g, 62.12 mmol) and H₂O (5 mL) in EtOH (50 mL) was added NH₄Cl (6.65 g, 124.25 mmol), then the mixture was stirred at about 80° C. for about 12 h. TLC (petroleum ether:ethyl acetate=3:1) indicated starting material was consumed completely, and one major new spot was detected. LCMS showed mass of the desired compound. The reaction was filtered and concentrated under reduced pressure, then added water (100 mL), then extracted with ethyl acetate (100 mL*3) and dried over anhydrous sodium sulfate, filtered and concentrated under reduced pressure to give a residue. The residue was purified by flash chromatography (silica gel, petroleum ether/ethyl acetate=1/0 to 5/1) to give 8-bromo-7-fluoro-chroman-6-amine (2.21 g, 8.98 mmol).

¹H NMR (400 MHz, METHANOL-d₄) δ:1.87-2.00 (m, 2H) 2.70 (t, J=6.50 Hz, 2H) 4.09-4.24 (m, 2H) 6.56 (d, J=9.51 Hz, 1H)

### Step 7. tert-butyl 5-(6-amino-7-fluoro-chroman-8-yl)-2,3,4,7-tetrahydroazepine-1-carboxylate

To a solution of 8-bromo-7-fluoro-chroman-6-amine (70 mg, 284.47 μmol) and tert-butyl 5-(4,4,5,5-tetramethyl-1,3,2-dioxaborolan-2-yl)-2,3,4,7-tetrahydroazepine-1-carboxylate (91.95 mg, 284.47 μmol) in dioxane (3 mL) and H₂O (0.3 mL) was added K₂CO₃ (78.63 mg, 568.93 μmol) and Pd(dppf)Cl₂ (20.81 mg, 28.45 μmol), then the mixture was stirred at about 100° C. for about 12 h under an atmosphere of nitrogen. TLC (petroleum ether:ethyl acetate=3:1) indicated starting material was consumed completely, and one major new spot was detected. LCMS showed starting material was consumed completely and mass of the desired compound. The reaction was filtered and concentrated under reduced pressure to give a residue. The residue was purified by prep-TLC (SiO₂, petroleum ether:ethyl acetate=3:1) to give tert-butyl 5-(6-amino-7-fluoro-chroman-8-yl)-2,3,4,7-tetrahydroazepine-1-carboxylate (70 mg, 193.14 μmol).

### Step 8. tert-butyl 5-[7-fluoro-6-[[4-methyl-6-(methylamino)pyrimidin-2-yl]amino]chroman-8-yl]-2,3,4,7-tetrahydroazepine-1-carboxylate

To a solution of tert-butyl 5-(6-amino-7-fluoro-chroman-8-yl)-2,3,4,7-tetrahydroazepine-1-carboxylate (55 mg, 151.75 μmol) and 2-chloro-N,6-dimethyl-pyrimidin-4-amine (25.11 mg, 159.34 μmol) in i-PrOH (2.00 mL) was added TFA (1.73 mg, 15.18 μmol, 1.17 μL), then the mixture was stirred at about 130° C. for about 3 h. LCMS showed starting material was consumed completely and mass of the desired compound. The reaction was filtered and concentrated under reduced pressure to give tert-butyl 5-[7-fluoro-6-[[4-methyl-6-(methylamino)pyrimidin-2-yl]amino]chroman-8-yl]-2,3,4,7-tetrahydroazepine-1-carboxylate (70 mg, crude).

### Step 9. N2-[7-fluoro-8-(2,3,4,7-tetrahydro-1H-azepin-5-yl)chroman-6-yl]-N4,6-dimethyl-pyrimidine-2,4-diamine

To a solution of tert-butyl 5-[7-fluoro-6-[[4-methyl-6-(methylamino)pyrimidin-2-yl]amino]chroman-8-yl]-2,3,4,7-tetrahydroazepine-1-carboxylate (70 mg, 144.75 μmol) in DCM (2 mL) was added ZnBr₂ (325.98 mg, 1.45 mmol), then the mixture was stirred at about 35° C. for about 12 h. LCMS showed starting material was consumed completely and mass of the desired compound. The reaction was concentrated under reduced pressure to give a residue. The residue was purified by prep-HPLC (TFA condition, column: C¹⁸⁻¹ 150*30 mm*5 um; mobile phase: [water(TFA)-ACN; B %: 1%-40%, 8 min) to give N2-[7-fluoro-8-(2,3,4,7-tetrahydro-1H-azepin-5-yl)chroman-6-yl]-N4,6-dimethyl-pyrimidine-2,4-diamine (45.3 mg, 91.24 μmol, TFA) (purity: 100%).

¹H NMR (400 MHz, METHANOL-d₄) δ:1.96-2.04 (m, 2H) 2.05-2.12 (m, 2H) 2.28 (s, 3H) 2.61-2.69 (m, 2H) 2.80 (br t, J=6.13 Hz, 2H) 2.90 (s, 3H) 3.44-3.52 (m, 2H) 3.90 (br d, J=6.38 Hz, 2H) 4.19-4.26 (m, 2H) 5.86 (t, J=6.25 Hz, 1H) 5.98 (s, 1H) 7.30 (br d, J=8.50 Hz, 1H)

### EXAMPLE 51. Synthesis of Compound 223

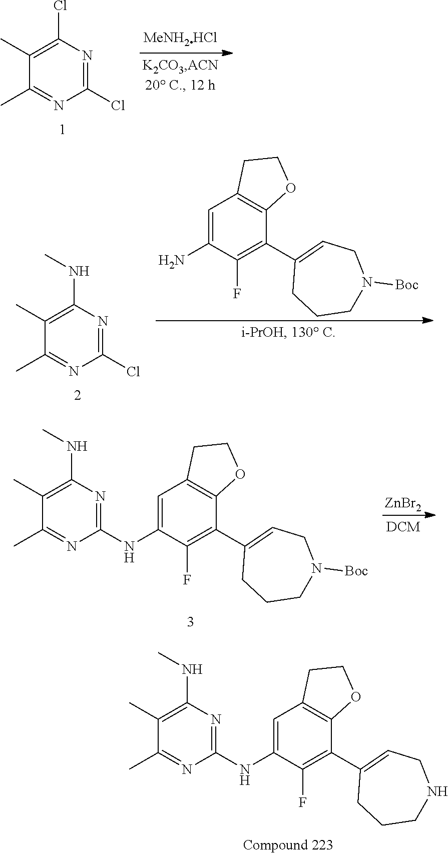

### Step 1. 2-chloro-N,5,6-trimethyl-pyrimidin-4-amine

To a solution of 2,4-dichloro-5,6-dimethyl-pyrimidine (200 mg, 1.13 mmol) and methanamine;hydrochloride (228.83 mg, 3.39 mmol) in ACN (10 mL) was added K₂CO₃ (780.69 mg, 5.65 mmol), then the mixture was stirred at about 20° C. for about 12 h. LCMS showed starting material was consumed completely and mass of the desired compound. The reaction was concentrated under reduced pressure, then added into water (100 mL), then extracted with ethyl acetate (50 mL*3). The combined organic layers were washed with brine (50 mL) and dried over anhydrous sodium sulfate. It was filtered and concentrated under reduced pressure to give 2-chloro-N,5,6-trimethyl-pyrimidin-4-amine (150 mg, crude).

### Step 2. tert-butyl 5-[5-[[4,5-dimethyl-6-(methylamino)pyrimidin-2-yl]amino]-6-fluoro-2,3-dihydrobenzofuran-7-yl]-2,3,4,7-tetrahydroazepine-1-carboxylate

The mixture of 2-chloro-N,5,6-trimethyl-pyrimidin-4-amine (50 mg, 291.33 μmol) and tert-butyl 5-(5-amino-6-fluoro-2,3-dihydrobenzofuran-7-yl)-2,3,4,7-tetrahydroazepine-1-carboxylate (101.50 mg, 291.33 mol) in i-PrOH (2 mL) was stirred at about 130° C. for about 3 h. LCMS showed the reaction was complete mostly and the desired mass was detected. The reaction mixture was concentrated to give tert-butyl 5-[5-[[4,5-dimethyl-6-(methylamino) pyrimidin-2-yl]amino]-6-fluoro-2,3-dihydrobenzofuran-7-yl]-2,3,4,7-tetrahydroazepine-1-carboxylate (140 mg, crude).

### Step 3. N2-[6-fluoro-7-(2,3,4,7-tetrahydro-1H-azepin-5-yl]-2,3-dihydrobenzofuran-5-yl-N4,5,6-trimethyl-pyrimidine-2,4-diamine

To a mixture of tert-butyl 5-[5-[[4,5-dimethyl-6-(methylamino)pyrimidin-2-yl]amino]-6-fluoro-2,3-dihydrobenzofuran-7-yl]-2,3,4,7-tetrahydroazepine-1-carboxylate (140 mg, 289.51 mol) in DCM (2 mL) was added ZnBr₂ (651.97 mg, 2.90 mmol) stirred at about 20° C. for about 12 hrs. LCMS showed the reaction was complete mostly and the desired mass was detected. The reaction was filtered and concentrated under reduced pressure to give a residue. The residue was purified by prep-HPLC(TFA condition:Phenomenex Luna 80*30 mm*3 um; mobile phase:water (TFA)-ACN; B %: 1%-30%,8 min) to give N2-[6-fluoro-7-(2,3,4,7-tetrahydro-1H-azepin-5-yl)-2,3-dihydrobenzofuran-5-yl]-N4,5,6-trimethyl-pyrimidine-2,4-diamine (8.1 mg, 21.12 μmol).

¹H NMR (400 MHz, METHANOL-d₄) δ: 1.99 (s, 3H) 2.08 (br s, 2H) 2.33 (s, 3H) 2.71-2.78 (m, 2H) 2.94 (s, 3H) 3.24 (t, J=8.69 Hz, 2H) 3.48 (br s, 2H) 3.92 (br d, J=4.00 Hz, 2H) 4.65 (t, J=8.76 Hz, 2H) 6.03 (br s, 1H) 7.42 (br d, J=7.25 Hz, 1H)

### EXAMPLE 52. Synthesis of Compound 280 and Compound 291

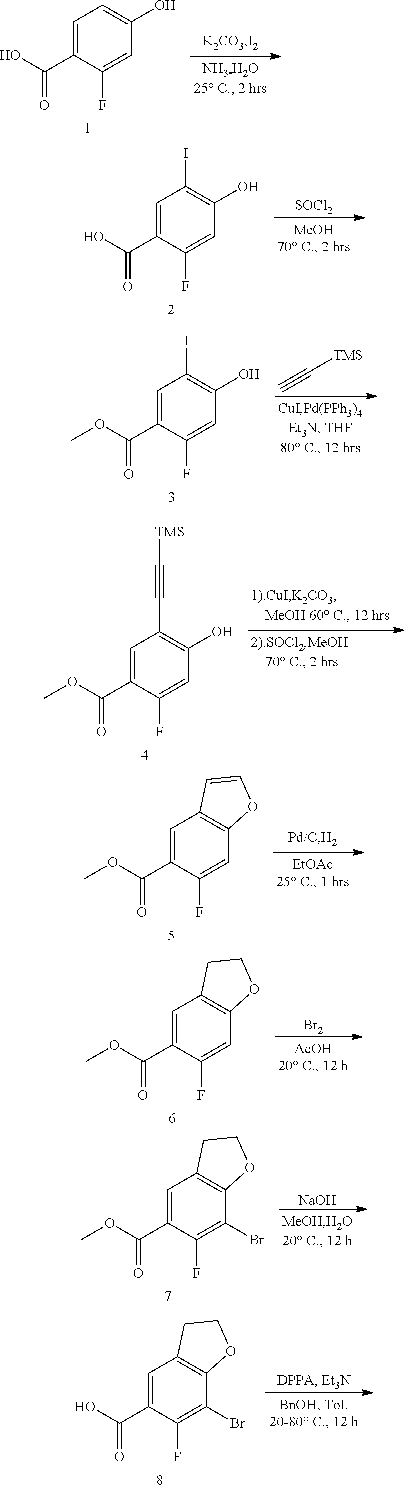

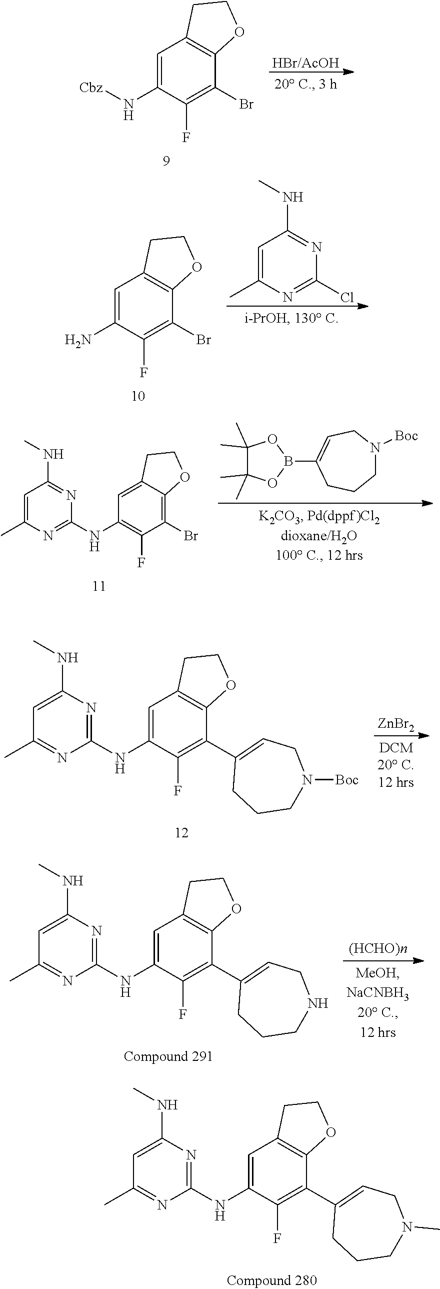

### Step 1. 2-fluoro-4-hydroxy-5-iodo-benzoic acid

K₂CO₃ (13.28 g, 96.09 mmol) was dissolved in NH₃·H₂O (13 mL), 12 (8.13 g, 32.03 mmol) was added, then the mixture was stirred at about 25° C. for about 1 hr. Then 2-fluoro-4-hydroxy-benzoic acid (5 g, 32.03 mmol) was added to the mixture was stirred at about 25° C. for about 2 hrs. LCMS showed the reaction was complete mostly and the desired mass was detected. The reaction mixture was acidified by HCl (2 M) till pH=1. The reaction mixture was added to water (20 mL), extracted with EtOAc (100 mL*3). The combined organic layers were washed with 200 mL of brine, dried over Na₂SO₄, filtered and concentrated under reduced pressure to 2-fluoro-4-hydroxy-5-iodo-benzoic acid (9 g, crude).

### Step 2. methyl 2-fluoro-4-hydroxy-5-iodo-benzoate

To a mixture of 2-fluoro-4-hydroxy-5-iodo-benzoic acid (9 g, 31.91 mmol) in MeOH (200 mL) was added SOCl₂ (10 mL) at about 0° C., then the mixture was stirred at about 70° C. for about 2 hrs. LCMS showed the reaction was complete mostly and the desired mass was detected. The reaction mixture was added to water (100 mL), extracted with EtOAc (200 mL*3). The combined organic layers were washed with 300 mL of brine, dried over Na₂SO₄, filtered and concentrated under reduced pressure to give methyl 2-fluoro-4-hydroxy-5-iodo-benzoate (10.55 g, crude).

### Step 3. methyl 2-fluoro-4-hydroxy-5-(2-trimethylsilylethynyl)benzoate

A mixture of methyl 2-fluoro-4-hydroxy-5-iodo-benzoate (7.8 g, 26.35 mmol), ethynyl(trimethyl)silane (3.11 g, 31.62 mmol, 4.47 mL), TEA (13.33 g, 131.74 mmol, 18.36 mL), palladium;triphenylphosphane (3.04 g, 2.63 mmol), and CuI (501.80 mg, 2.63 mmol, 89.29 μL) was added to solvent DMF (80 mL). The suspension was degassed and purged with N2 for 3 times. The mixture was stirred under N2 at about 80° C. for about 12 hrs. LCMS showed the reaction was complete mostly and the desired mass was detected. The reaction mixture was added to water (50 mL), extracted with EtOAc (150 mL*3). The combined organic layers were washed with 300 mL of brine, dried over Na₂SO₄, filtered and concentrated under reduced pressure to give a residue. The crude product was purified by column chromatography on silica gel (Petroleum ether:Ethyl acetate=1:0-20:1) to give methyl 2-fluoro-4-hydroxy-5-(2-trimethylsilylethynyl)benzoate (2 g, crude).

### Step 4. methyl 6-fluorobenzofuran-5-carboxylate

To a mixture of methyl 2-fluoro-4-hydroxy-5-(2-trimethylsilylethynyl)benzoate (2 g, 7.51 mmol) in MeOH (31.06 mL) was added N-ethyl-N-isopropyl-propan-2-amine (1.94 g, 15.02 mmol, 2.62 mL), CuI (143.01 mg, 750.92 μmol, 25.45 μL) at about 25° C., the mixture was stirred at about 60° C. for about 4 hrs. Then K₂CO₃ (2.08 g, 15.02 mmol) was added to the mixture stirred at about 60° C. for about 12 hrs. LCMS showed the reaction was complete mostly and the desired mass was detected. The reaction mixture was filtered and concentrated to give the crude product. The crude product was purified by reversed-phase HPLC (0.10% TFA condition) to give 6-fluorobenzofuran-5-carboxylic acid (3.4 g, crude).

To a mixture of 6-fluorobenzofuran-5-carboxylic acid (3.3 g, 18.32 mmol) in MeOH (30 mL) was added SOCl₂ (3 mL) at about 0° C., then the mixture was stirred at about 70° C. for about 2 hrs. LCMS showed the reaction was complete mostly and the desired mass was detected. The reaction mixture was added to water (50 mL), extracted with EtOAc (100 mL*3). The combined organic layers were washed with 200 mL of brine, dried over Na₂SO₄, filtered and concentrated under reduced pressure to give methyl 6-fluorobenzofuran-5-carboxylate (1.34 g, crude).

### Step 5. methyl 6-fluoro-2,3-dihydrobenzofuran-5-carboxylate

To a mixture of methyl 6-fluorobenzofuran-5-carboxylate (1.26 g, 6.49 mmol) in MeOH (2 mL) was added Pd/C (10% purity) under N2 at about 25° C., then the mixture was stirred at about 50° C. for about 1 hr under H₂ (15 psi). LCMS showed the reaction was complete mostly and the desired mass was detected. The reaction mixture was filtered and concentrated to give methyl 6-fluoro-2,3-dihydrobenzofuran-5-carboxylate (1.27 g, crude).

### Step 6. methyl 7-bromo-6-fluoro-2,3-dihydrobenzofuran-5-carboxylate

To a mixture of methyl 6-fluoro-2,3-dihydrobenzofuran-5-carboxylate (1.27 g, 6.47 mmol) in AcOH (10 mL) was added Br₂ (3.10 g, 19.42 mmol) at about 0° C., then the mixture was stirred at about 20° C. for about 12 hrs. LCMS showed the reaction was complete mostly and the desired mass was detected. The reaction mixture was added to ice water (50 mL), and filtered and the filter cake was dried in vacuum to give methyl 7-bromo-6-fluoro-2,3-dihydrobenzofuran-5-carboxylate (1.56 g, crude).

### Step 7. 7-bromo-6-fluoro-2,3-dihydrobenzofuran-5-carboxylic acid

To a solution of methyl 7-bromo-6-fluoro-2,3-dihydrobenzofuran-5-carboxylate (1.56 g, 5.67 mmol) in H₂O (5 mL), MeOH (20 mL) was added NaOH (453.67 mg, 11.34 mmol, 212.99 L). The reaction mixture was stirred at about 20° C. for about 12 hrs. LCMS showed the reaction was complete mostly and the desired mass was detected. It was concentrated under reduced pressure, then added dropwise 1N HCl to pH=1. It was filtered to get a filter cake. Compound 7-bromo-6-fluoro-2,3-dihydrobenzofuran-5-carboxylic acid (1.25 g, crude) was obtained.

### Step 8. benzyl N-(7-bromo-6-fluoro-2,3-dihydrobenzofuran-5-yl)carbamate

To a solution of 7-bromo-6-fluoro-2,3-dihydrobenzofuran-5-carboxylic acid (1.15 g, 4.41 mmol) in toluene (12 mL) was added DPPA (1.29 g, 5.29 mmol), TEA (1.34 g, 13.22 mmol, 1.84 mL). The reaction mixture was stirred at about 20° C. for about 1 h. BnOH (592.73 mg, 13.22 mmol) was added to the above mixture then the reaction mixture was stirred at about 80° C. for about 12 hrs. LCMS showed the reaction was complete mostly and the desired mass was detected. The reaction mixture was filtered and concentrated to give the crude product. The crude product was purified by column chromatography on silica gel (Petroleum ether:Ethyl acetate=1:0-10:1) to give benzyl N-(7-bromo-6-fluoro-2,3-dihydrobenzofuran-5-yl)carbamate (1.2 g, crude).

¹H NMR (400 MHz, METHANOL-d₄) δ: 7.48-7.28 (m, 6H), 5.17 (s, 2H), 4.66 (t, J=8.8 Hz, 2H), 3.30-3.24 (m, 2H)

### Step 9. 7-bromo-6-fluoro-2,3-dihydrobenzo furan-5-amine

To a mixture of benzyl N-(7-bromo-6-fluoro-2,3-dihydrobenzofuran-5-yl)carbamate (300 mg, 819.27 μmol) in AcOH (2 mL) was added HBr (4 mL) at about 25° C., then the mixture was stirred at about 25° C. for about 3 hrs. LCMS showed starting material was consumed completely and mass of the desired compound. The reaction was extracted with dichloromethane (3×20 mL). To the aqueous phase was added NaOH to pH=8 at about 0° C., then it was extracted with dichloromethane (3×20 mL). The combined organic layers were washed with brine (3×30 mL) and dried over anhydrous sodium sulfate. It was filtered and concentrated under reduced pressure to give 7-bromo-6-fluoro-2,3-dihydrobenzofuran-5-amine (140 mg, crude).

### Step 10. 7-bromo-6-fluoro-2,3-dihydrobenzofuran-5-amine

To a mixture of 7-bromo-6-fluoro-2,3-dihydrobenzofuran-5-amine (20 mg, 86.19 μmol) in i-PrOH (1 mL) was added 2-chloro-N,6-dimethyl-pyrimidin-4-amine (13.58 mg, 86.19 μmol) and TFA (982.75 μg, 8.62 μmol, 6.64e-1 μL), then the mixture was stirred at about 130° C. for about 1 hr. LCMS showed starting material was consumed completely and mass of the desired compound. The reaction mixture was filtered to afford 7-bromo-6-fluoro-2,3-dihydrobenzofuran-5-amine (20 mg, 86.19 μmol).

### Step 11. tert-butyl 5-[6-fluoro-5-[[4-methyl-6-(methylamino)pyrimidin-2-yl]amino]-2,3-dihydrobenzo furan-7-yl]-2,3,4,7-tetrahydroazepine-1-carboxylate

To a solution of N2-(7-bromo-6-fluoro-2,3-dihydrobenzofuran-5-yl)-N4,6-dimethyl-pyrimidine-2,4-diamine (22 mg, 62.29 mol) in H₂O (0.1 mL) and dioxane (1 mL) was added tert-butyl 5-(4,4,5,5-tetramethyl-1,3,2-dioxaborolan-2-yl)-2,3,4,7-tetrahydroazepine-1-carboxylate (20.13 mg, 62.29 mol), K₂CO₃ (25.83 mg, 186.87 μmol), and cyclopentyl(diphenyl)phosphane;dichloropalladium;iron (4.56 mg, 6.23 μmol) under N2 atmosphere. The reaction mixture was stirred at about 100° C. for about 12 hr under N2 atmosphere. TLC (ethyl acetate:MeOH=10:1) indicated starting material remained and one major new spot was detected. The reaction mixture was concentrated under reduced pressure to give a residue and then purified by prep-TLC (ethyl acetate:MeOH=5:1) to give tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (20 mg, 42.59 μmol).

### Step 12. N2-[6-fluoro-7-(2, 3, 4, 7-tetrahydro-1H-azepin-5-yl]-2,3-dihydrobenzofuran-5-yl]-N4,6-dimethyl-pyrimidine-2,4-diamine

To a mixture of tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (22.3 mg, 47.49 mol) in DCM (1 mL) was added ZnBr₂ (53.48 mg, 237.46 mol). The reaction mixture was stirred at about 25° C. for about 12 hr. LCMS showed starting material was consumed completely and mass of the desired compound. The reaction mixture was concentrated under reduced pressure to give a residue and then purified by prep-HPLC (TFA condition, column: Phenomenex Luna 80*30 mm*3 um; mobile phase: [water(TFA)-ACN]; B %: 1%-35%, 8 min, LC-mass to give N2-[6-fluoro-7-(2,3,4,7-tetrahydro-1H-azepin-5-yl)-2,3-dihydrobenzofuran-5-yl]-N4,6-dimethyl-pyrimidine-2,4-diamine (12.5 mg, 33.84 μmol, 71.24% yield) (TFA salt).

¹H NMR (400 MHz, METHANOL-d₄) δ: 7.37 (br d, J=7.3 Hz, 1H), 6.03 (t, J=6.4 Hz, 1H), 5.98 (s, 1H), 4.68-4.62 (m, 2H), 3.92 (d, J=6.4 Hz, 2H), 3.53-3.44 (m, 2H), 3.24 (t, J=8.6 Hz, 2H), 2.93-2.87 (m, 3H), 2.77-2.71 (m, 2H), 2.28 (s, 3H), 2.12-2.02 (m, 2H) MS (ESI): m/z=370.2 [M+H].

### Step 13. N2-[6-fluoro-7-(1-methyl-2,3,4,7-tetrahydroazepin-5-yl]-2,3-dihydrobenzofuran-5-yl]-N4,6-dimethyl-pyrimidine-2,4-diamine

To a solution of N2-[6-fluoro-7-(2,3,4,7-tetrahydro-1H-azepin-5-yl)-2,3-dihydrobenzofuran-5-yl]-N4,6-dimethyl-pyrimidine-2,4-diamine (32 mg, 86.62 μmol) in MeOH (1 mL) was added AcOH (520.16 μg, 8.66 mol) to adjust pH to 5, then NaBH₃CN (10.89 mg, 173.24 μmol) and (HCHO)ₙ(7.80 mg, 259.86 μmol) was added to above solution. The mixture was stirred at about 20° C. for about 12 hrs. LCMS showed starting material was consumed completely and mass of the desired compound. It was concentrated under reduced pressure to give a residue. Then, it was purified by prep-HPLC (TFA condition, column: Phenomenex Luna 80*30 mm*3 um; mobile phase: [water(TFA)-ACN]; B %: 1%-35%, 8 min, LC-MS to give N2-[6-fluoro-7-(1-methyl-2,3,4,7-tetrahydroazepin-5-yl)-2,3-dihydrobenzofuran-5-yl]-N4,6-dimethyl-pyrimidine-2,4-diamine (15.7 mg, 40.94 μmol) (TFA salt, 100.0% purity).

¹H NMR (400 MHz, METHANOL-d₄) δ: 7.41 (d, J=7.4 Hz, 1H), 6.03-5.98 (m, 2H), 4.69 (dt, J=1.9, 8.7 Hz, 2H), 4.16-4.09 (m, 1H), 4.05-3.96 (m, 1H), 3.78-3.67 (m, 1H), 3.53-3.44 (m, 1H), 3.30-3.24 (m, 2H), 2.95 (s, 3H), 2.92 (s, 3H), 2.83-2.68 (m, 2H), 2.30 (s, 3H), 2.19-2.08 (m, 2H) MS (ESI): m/z=354.2 [M+H]

### EXAMPLE 53. Synthesis of Compound 280 (alternative synthesis)

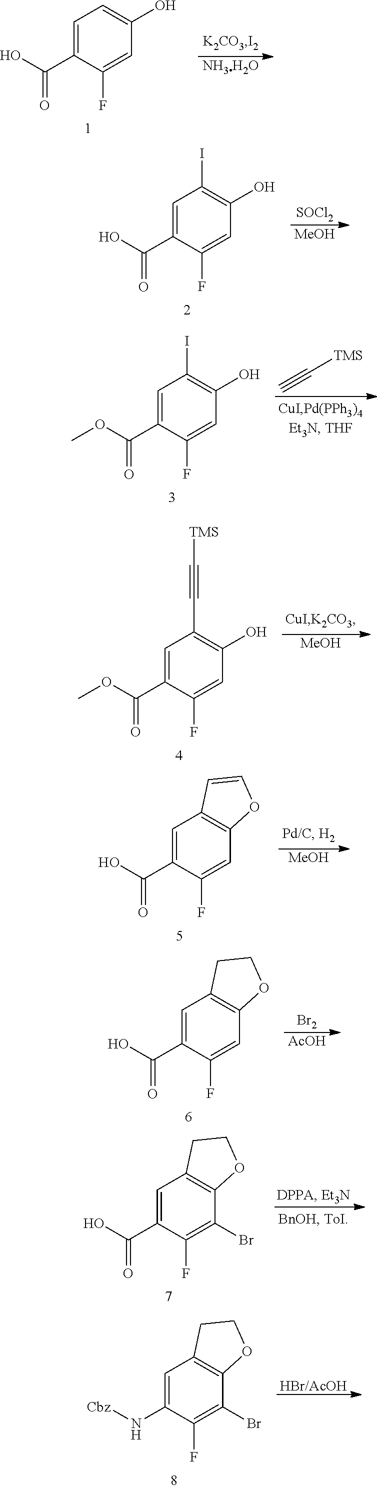

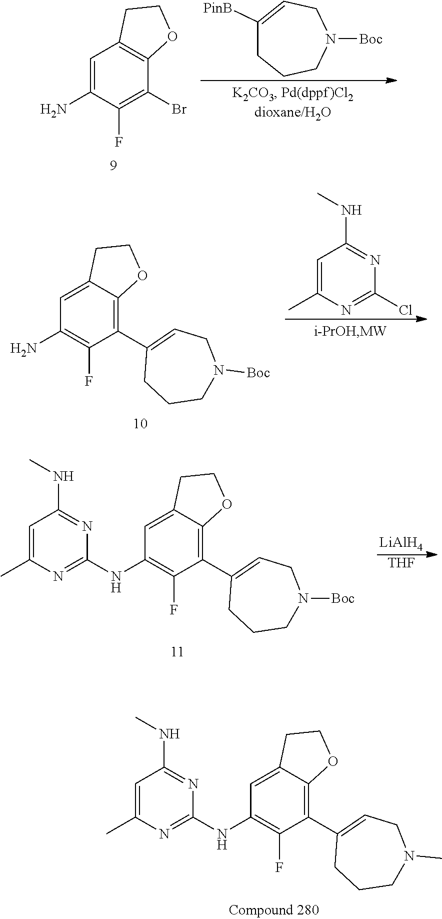

### Step 1. tert-butyl N-(1-methyl-2-oxo-ethyl)carbamate

K₂CO₃ (53.12 g, 384.34 mmol) was dissolved in NH₃·H₂O (300 mL), 12 (32.52 g, 128.11 mmol) was added, then the mixture was stirred at about 25° C. for about 1 hr. Then 2-fluoro-4-hydroxy-benzoic acid (20 g, 128.11 mmol) was added to the mixture was stirred at about 25° C. for about 2 hrs. LCMS showed the reaction was complete mostly and the desired mass was detected. The reaction mixture was acidified by HCl (2 M) till pH=1. The reaction mixture was added to water (200 mL), extracted with EtOAc (500 mL*3). The combined organic layers were washed with 500 mL of brine, dried over Na₂SO₄, filtered and concentrated under reduced pressure to afford 2-fluoro-4-hydroxy-5-iodo-benzoic acid (36 g, crude).

¹H NMR (400 MHz, METHANOL-d₄) δ:8.25 (d, J=8.3 Hz, 1H), 6.61 (d, J=12.3 Hz, 1H)

### Step 2. 2-fluoro-4-hydroxy-5-iodo-benzoate

To a mixture of 2-fluoro-4-hydroxy-5-iodo-benzoic acid (36 g, 127.66 mmol) in MeOH (400 mL) was added SOCl₂ (20 mL) at about 0° C., then the mixture was stirred at about 70° C. for about 2 hrs. LCMS showed the reaction was complete mostly and the desired mass was detected. The reaction mixture was added to water (300 mL), extracted with EtOAc (500 mL*3). The combined organic layers were washed with 800 mL of brine, dried over Na₂SO₄, filtered and concentrated under reduced pressure to give methyl 2-fluoro-4-hydroxy-5-iodo-benzoate (33.6 g, crude).

### Step 3. methyl 2-fluoro-4-hydroxy-5-(2-trimethylsilylethynyl)benzoate

A mixture of methyl 2-fluoro-4-hydroxy-5-iodo-benzoate (5 g, 16.89 mmol), ethynyl-(trimethyl)silane (8.29 g, 84.45 mmol, 11.93 mL), TEA (10.25 g, 101.34 mmol, 14.12 mL), palladium;triphenylphosphane (1.95 g, 1.69 mmol), CuI (1.13 g, 5.91 mmol, 200.33 μL) were added to solvent DMF (49.86 mL). The suspension was degassed and purged with N₂ for 3 times. The mixture was stirred under N2 at about 50° C. for about 12 hrs. TLC (petroleum ether:ethyl acetate=5:1) indicated methyl 2-fluoro-4-hydroxy-5-iodo-benzoate was consumed completely, and major new spots was detected. The mixture was concentrated in vacuum. The residue was purified by flash chromatography (silica gel, petroleum ether/ethyl acetate=50/1 to 10/1) to give methyl 2-fluoro-4-hydroxy-5-(2-trimethylsilylethynyl)benzoate (2 g, 7.51 mmol).

### Step 4. 6-fluoro-2,3-dihydrobenzofuran-5-carboxylic acid

To a mixture of methyl 2-fluoro-4-hydroxy-5-(2-trimethylsilylethynyl)benzoate (2 g, 7.51 mmol) in MeOH (20 mL) was added DIEA (1.94 g, 15.02 mmol, 2.62 mL), CuI (143.01 mg, 750.92 μmol, 25.45 μL) at about 25° C., the mixture was stirred at about 60° C. for about 2 hrs. Then K₂CO₃ (2.08 g, 15.02 mmol) was added to the mixture stirred at about 60° C. for about 12 hrs. LCMS showed the reaction was complete and the desired mass was detected. It was concentrated under reduced pressure, and then diluted by 50 mL H₂O, then added dropwise conc. HCl to pH=1. The suspension was extracted with EtOAc (10 mL*5), and then the organic phase was concentrated in vacuum. 6-fluoro-2,3-dihydrobenzofuran-5-carboxylic acid (1.6 g, crude) was obtained.

### Step 5. methyl 6-fluoro-2,3-dihydrobenzofuran-5-carboxylate

To a mixture of methyl 6-fluorobenzofuran-5-carboxylate (20 g, 103.01 mmol) in MeOH (400 mL) was added Pd/C (10 g, 10% purity) under N2 at about 25° C., then the mixture was stirred at about 50° C. for about 12 hr under H₂ (15 psi). LCMS showed the reaction was complete and the desired mass was detected. The reaction mixture was filtered and concentrated. methyl 6-fluoro-2,3-dihydrobenzofuran-5-carboxylate (18 g, 91.75 mmol) was obtained.

### Step 6. 7-bromo-6-fluoro-2,3-dihydrobenzo furan-5-carboxylic acid

To a mixture of 6-fluoro-2,3-dihydrobenzofuran-5-carboxylic acid (18 g, 98.82 mmol) in AcOH (180 mL) was added dropwise Br₂ (47.38 g, 296.46 mmol, 15 mL) at about 0° C., then the mixture was stirred at about 25° C. for about 12 hrs. The mixture was concentrated in vacuo at about 45° C. 7-bromo-6-fluoro-2,3-dihydrobenzofuran-5-carboxylic acid (25 g, crude) was obtained.

### Step 7. benzyl N-(7-bromo-6-fluoro-2,3-dihydrobenzofuran-5-yl)carbamate

To a solution of 7-bromo-6-fluoro-2,3-dihydrobenzofuran-5-carboxylic acid (25 g, 95.77 mmol) in toluene (250 mL) was added DPPA (27.95 g, 114.92 mmol), TEA (29.07 g, 287.31 mmol, 40.04 mL). It was stirred at about 20° C. for about 1 h. Then to it was added BnOH (12.89 g, 287.31 mmol). It was stirred at about 80° C. for about 12 hrs. TLC (petroleum ether:ethyl acetate=3:1, Rf=0.5) indicated starting material was consumed completely, and one major new spot was detected. The reaction mixture was concentrated to give the crude product. The crude product was purified by column chromatography on silica gel (Petroleum ether:Ethyl acetate=1:0) benzyl N-(7-bromo-6-fluoro-2,3-dihydrobenzofuran-5-yl)carbamate (33 g, 90.12 mmol) was obtained.

### Step 8. 7-bromo-6-fluoro-2,3-dihydrobenzofuran-5-amine

To a mixture of benzyl N-(7-bromo-6-fluoro-2,3-dihydrobenzofuran-5-yl)carbamate (31 g, 84.66 mmol) in AcOH (100 mL) was added HBr (100 mL) at about 25° C., then the mixture was stirred at about 25° C. for about 3 hrs. LCMS showed starting material was consumed completely and desired compound was detected. The reaction was filtered, and then the filter cake was washed by EtOAc (10 ml*2), and then dissolved in H₂O (100 mL), and then to the solution was added sat. aq. Na₂CO₃ to pH=7, and then extracted with EtOAc (40 mL*3), and then the organic phase was concentrated in vacuum to give 7-bromo-6-fluoro-2,3-dihydrobenzofuran-5-amine (13.5 g, 58.18 mmol).

### Step 9. tert-butyl 5-(5-amino-6-fluoro-2,3-dihydrobenzofuran-7-yl)-2,3,4,7-tetrahydroazepine-1-carboxylate

To a solution of 7-bromo-6-fluoro-2,3-dihydrobenzofuran-5-amine (5.98 g, 25.77 mmol) in dioxane (100 mL) and H₂O (10 mL) was added tert-butyl 5-(4,4,5,5-tetramethyl-1,3,2-dioxaborolan-2-yl)-2,3,4,7-tetrahydroazepine-1-carboxylate (8.33 g, 25.77 mmol), K₂CO₃ (10.68 g, 77.31 mmol) and cyclopentyl(diphenyl)phosphane;dichloropalladium;iron (1.89 g, 2.58 mmol) under N₂ atmosphere. The reaction mixture was stirred at about 100° C. for about 12 h under N₂ atmosphere. LCMS showed the reaction was complete and the desired mass was detected. The reaction was concentrated under reduced pressure to give a residue. The residue was purified by column chromatography (SiO₂, Petroleum ether/Ethyl acetate=1/0 to 12/1). tert-butyl 5-(5-amino-6-fluoro-2,3-dihydrobenzofuran-7-yl)-2,3,4,7-tetrahydroazepine-1-carboxylate (8 g, crude) was obtained.

### Step 10. tert-butyl 5-[6-fluoro-5-[[4-methyl-6-(methylamino)pyrimidin-2-yl amino 1-2,3-dihydrobenzo furan-7-yl]-2,3,4,7-tetrahydroazepine-1-carboxylate

The mixture of 2-chloro-N,6-dimethyl-pyrimidin-4-amine (1.81 g, 11.48 mmol) and tert-butyl 5-(5-amino-6-fluoro-2,3-dihydrobenzofuran-7-yl)-2,3,4,7-tetrahydroazepine-1-carboxylate (4 g, 11.48 mmol) in i-PrOH (110 mL) was stirred at about 130° C. for about 5 hrs. LCMS showed the reaction was complete and the desired mass was detected. The reaction was concentrated under reduced pressure to give tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (6 g, crude).

### Step 11. N2-[6-fluoro-7-(1-methyl-2,3,4,7-tetrahydroazepin-5-yl]-2,3-dihydrobenzofuran-5-yl]-N4,6-dimethyl-pyrimidine-2,4-diamine

To the solution of tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (9.2 g, 19.59 mmol) in THF (100 mL) was added lithium;alumanuide (3 g, 79.05 mmol) at about 0° C., then stirred at about 80° C. for about 15 hrs. LCMS showed the reaction was complete and the desired mass was detected. Then to the reaction was added Na₂SO₄·10H₂O slowly until no bubbles with keeping the temperature at about 0-10° C. The reaction was filtered and concentrated under reduced pressure to give a residue. The crude product was purified by prep-HPLC(HPLC; water(NH₃H₂O)-ACN:column:Waters Xbridge BEH C18 250*70 mm*10 um; mobile phase:water(NH₃H₂O)-ACN; B %: 25%-55%,20 min) to give N2-[6-fluoro-7-(1-methyl-2,3,4,7-tetrahydroazepin-5-yl)-2,3-dihydrobenzofuran-5-yl]-N4,6-dimethyl-pyrimidine-2,4-diamine (4.4 g, 11.47 mmol).

¹H NMR (400 MHz, METHANOL-d₄) δ: 7.82 (br d, J=7.8 Hz, 1H), 5.91 (t, J=6.3 Hz, 1H), 5.79 (s, 1H), 4.54 (t, J=8.7 Hz, 2H), 3.30-3.27 (m, 2H), 3.18 (t, J=8.7 Hz, 2H), 2.92-2.88 (m, 2H), 2.85 (s, 3H), 2.58-2.52 (m, 2H), 2.37 (s, 3H), 2.16 (s, 3H), 1.90-1.81 (m, 2H)

### EXAMPLE 54. Synthesis of Compound 289

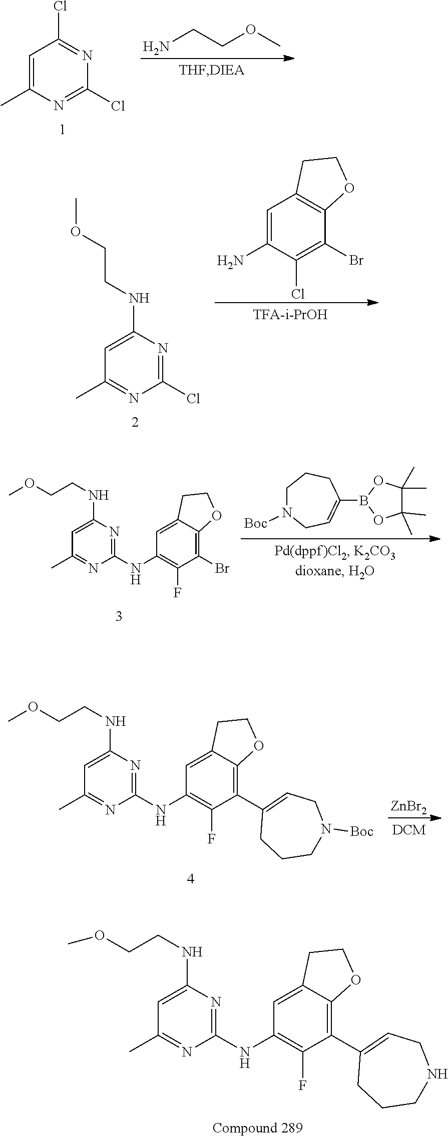

### Step 1. 2-chloro-N-(2-methoxyethyl)-6-methyl-pyrimidin-4-amine

To a mixture of 2-methoxyethanamine (13.82 g, 184.04 mmol, 16.00 mL), 2,4-dichloro-6-methyl-pyrimidine (10 g, 61.35 mmol) in THF (100 mL) was added N-ethyl-N-isopropyl-propan-2-amine (15.86 g, 122.70 mmol, 21.37 mL) at about 25° C., then the mixture was stirred at about 25° C. for about 12 hrs. LCMS showed the reaction was complete mostly and the desired mass was detected. The reaction mixture was filtered and concentrated to give the crude product. The crude product was purified by column chromatography on silica gel (Petroleum ether:Ethyl acetate=1:0-10:1) to give 2-chloro-N-(2-methoxyethyl)-6-methyl-pyrimidin-4-amine (2.7 g, crude).

### Step 2. N2-(7-bromo-6-fluoro-2,3-dihydrobenzofuran-5-yl)-N4-(2-methoxyethyl)-6-methyl-pyrimidine-2,4-diamine

To a solution of 7-bromo-6-fluoro-2,3-dihydrobenzofuran-5-amine (0.03 g, 129.28 mol) in i-PrOH (1 mL) was added 2-chloro-N-(2-methoxyethyl)-6-methyl-pyrimidin-4-amine (26.07 mg, 129.28 μmol) and TFA (1.47 mg, 12.93 μmol, 9.96e-1 μL). It was stirred at about 130° C. for about 1 hour. LCMS showed starting material was consumed completely and mass of the desired compound. It was added 1 mL of EtOAc and filtered to give a white solid. N2-(7-bromo-6-fluoro-2,3-dihydrobenzofuran-5-yl)-N4-(2-methoxyethyl)-6-methyl-pyrimidine-2,4-diamine (44 mg, crude) was obtained.

### Step 3. tert-butyl5-[6-fluoro-5-[[4-(2-methoxyethylamino)-6-methyl-pyrimidin-2-yl]amino]-2,3-dihydrobenzofuran-7-yl]-2,3,4,7-tetrahydroazepine-1-carboxylate

To a solution of tert-butyl 5-(4,4,5,5-tetramethyl-1,3,2-dioxaborolan-2-yl)-2,3,4,7-tetrahydroazepine-1-carboxylate (17.09 mg, 52.86 μmol) and N2-(7-bromo-6-fluoro-2,3-dihydrobenzofuran-5-yl)-N4-(2-methoxyethyl)-6-methyl-pyrimidine-2,4-diamine (21 mg, 52.86 mol), K₂CO₃ (21.92 mg, 158.59 μmol) in H₂O (0.1 mL) and dioxane (1 mL) was added Pd(dppf)Cl₂ (3.87 mg, 5.29 mol). The reaction mixture was stirred at about 100° C. for about 3 h under N2. LCMS showed starting material was consumed completely and mass of the desired compound. The reaction mixture was concentrated under reduced pressure to give a residue. The residue was purified by prep-TLC (ethyl acetate:methanol=5:1) to give tert-butyl 5-[6-fluoro-5-[[4-(2-methoxyethylamino)-6-methyl-pyrimidin-2-yl]amino]-2,3-dihydrobenzofuran-7-yl]-2,3,4,7tetrahydroazepine-1-carboxylate (87.4 mg, crude).

### Step 4. N2-[6-fluoro-7-(2,3,4,7-tetrahydro-1H-azepin-5-yl]-2,3-dihydrobenzofuran-5-yl]-N4-(2-methoxyethyl)-6-methyl-pyrimidine-2,4-diamine

To a solution of tert-butyl 5-[6-fluoro-5-[[4-(2-methoxyethylamino)-6-methyl-pyrimidin-2-yl]amino]-2,3-dihydrobenzofuran-7-yl]-2,3,4,7-tetrahydroazepine-1-carboxylate (30 mg, 58.41 μmol) in DCM (2 mL) was added ZnBr₂ (65.77 mg, 292.05 μmol). The reaction mixture was stirred at about 25° C. for about 12 hr. LCMS showed starting material was consumed completely and mass of the desired compound. The mixture was concentrated under reduced pressure and then purified by prep-HPLC (TFA condition, column: Phenomenex Luna 80*30 mm*3 um; mobile phase: [water(TFA)-ACN]; B %: 1%-30%, 8 min, HPLC) to give N2-[6-fluoro-7-(2,3,4,7-tetrahydro-1H-azepin-5-yl)-2,3-dihydrobenzofuran-5-yl]-N4-(2-methoxyethyl)-6-methyl-pyrimidine-2,4-diamine (11.5 mg, 27.81 mol) (TFA salt, 97.701% purity).

¹H NMR (400 MHz, METHANOL-d₄) δ: 2.08 (br d, J=4.38 Hz, 2H) 2.29 (s, 3H) 2.72-2.76 (m, 2H) 3.24 (t, J=8.69 Hz, 2H) 3.32 (s, 3H) 3.46-3.50 (m, 4H) 3.51 (br d, J=4.50 Hz, 2H) 3.92 (br d, J=6.13 Hz, 2H) 4.66 (t, J=8.76 Hz, 2H) 6.01 (s, 1H) 6.02-6.06 (m, 1H) 7.34 (br d, J=7.38 Hz, 1H)

### EXAMPLE 55. Synthesis of Compound 291 (alternative synthesis)

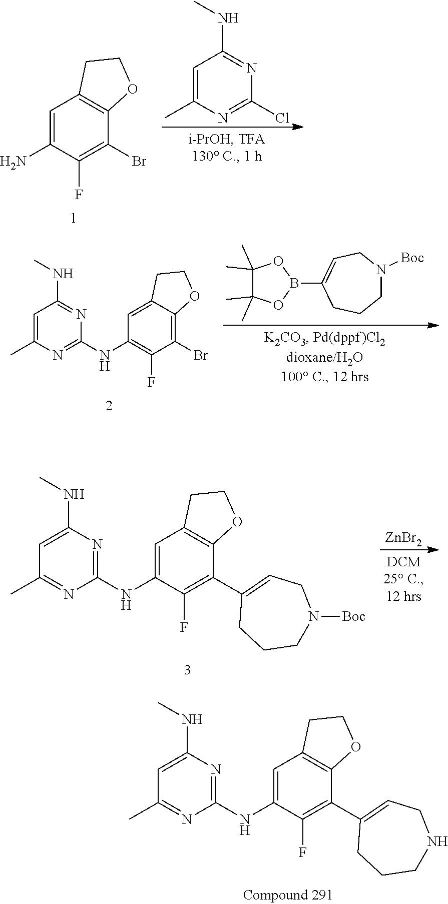

### Step 1. N2-(7-bromo-6-fluoro-2,3-dihydrobenzofuran-5-yl)-N4,6-dimethyl-pyrimidine-2,4-diamine

To a mixture of 7-bromo-6-fluoro-2,3-dihydrobenzofuran-5-amine (600 mg, 2.59 mmol) in i-PrOH (6 mL) was added 2-chloro-N,6-dimethyl-pyrimidin-4-amine (407.50 mg, 2.59 mmol) and TFA (29.48 mg, 258.57 μmol, 19.92 μL), then the mixture was stirred at about 130° C. for about 2 hr. LCMS showed starting material was consumed completely and mass of the desired compound. The reaction mixture was filtered to afford N2-(7-bromo-6-fluoro-2,3-dihydrobenzofuran-5-yl)-N4,6-dimethyl-pyrimidine-2,4-diamine (725 mg, crude).

### Step 2. tert-butyl 5-[6-fluoro-5-[[4-methyl-6-(methylamino)pyrimidin-2-yl]amino]-2,3-dihydrobenzo furan-7-yl]-2,3,4,7-tetrahydroazepine-1-carboxylate

To a mixture of N2-(7-bromo-6-fluoro-2,3-dihydrobenzofuran-5-yl)-N4,6-dimethyl-pyrimidine-2,4-diamine (725 mg, 2.05 mmol), tert-butyl 5-(4,4,5,5-tetramethyl-1,3,2-dioxaborolan-2-yl)-2,3,4,7-tetrahydroazepine-1-carboxylate (663.51 mg, 2.05 mmol) in dioxane (10 mL), H₂O (1 mL) was added K₂CO₃ (851.09 mg, 6.16 mmol), cyclopentyl(diphenyl)phosphane;dichloropalladium;iron (150.20 mg, 205.27 μmol) at about 25° C., then the mixture was stirred at about 100° C. for about 12 hrs under N₂. LCMS showed the reaction was complete mostly and the desired mass was detected. The reaction mixture was filtered and concentrated to give the crude product. The crude product was purified by column chromatography on silica gel (Petroleum ether:Ethyl acetate=1:0-1:1) to give tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (1 g, crude).

### Step 3. N2-[6-fluoro-7-(2,3,4,7-tetrahydro-1H-azepin-5-yl]-2,3-dihydrobenzofuran-5-yl]-N4,6-dimethyl-pyrimidine-2,4-diamine

To a mixture of tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (1 g, 2.13 mmol) in DCM (10 mL) was added ZnBr₂ (4.80 g, 21.30 mmol) at about 25° C., then the mixture was stirred at about 25° C. for about 12 hrs. LCMS showed the reaction was complete mostly and the desired mass was detected. The reaction mixture was filtered and concentrated to give the crude product. The crude product was purified by prep-HPLC (TFA condition; column: Phenomenex luna C18 250*50 mm*10 um; mobile phase: [water(0.1% TFA)-ACN]; B %: 5%-35%,10 min) to give N2-[6-fluoro-7-(2,3,4,7-tetrahydro-1H-azepin-5-yl)-2,3-dihydrobenzofuran-5-yl]-N4,6-dimethyl-pyrimidine-2,4-diamine (605.6 mg, 1.26 mmol, TFA).

¹H NMR (400 MHz, METHANOL-d₄) δ: 2.04-2.11 (m, 2H) 2.28 (s, 3H) 2.71-2.77 (m, 2H) 2.89 (s, 3H) 3.25 (t, J=8.76 Hz, 2H) 3.45-3.50 (m, 2H) 3.92 (d, J=6.38 Hz, 2H) 4.63-4.69 (m, 2H) 5.98 (d, J=0.75 Hz, 1H) 6.04 (t, J=6.32 Hz, 1H) 7.38 (br d, J=7.25 Hz, 1H)

### EXAMPLE 56. Synthesis of Compound 291 (alternative synthesis)

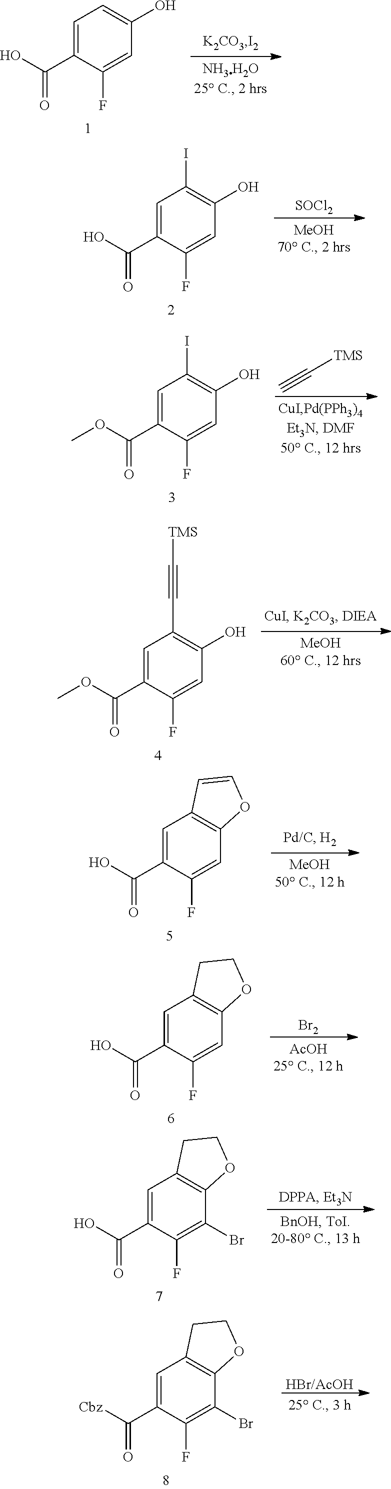

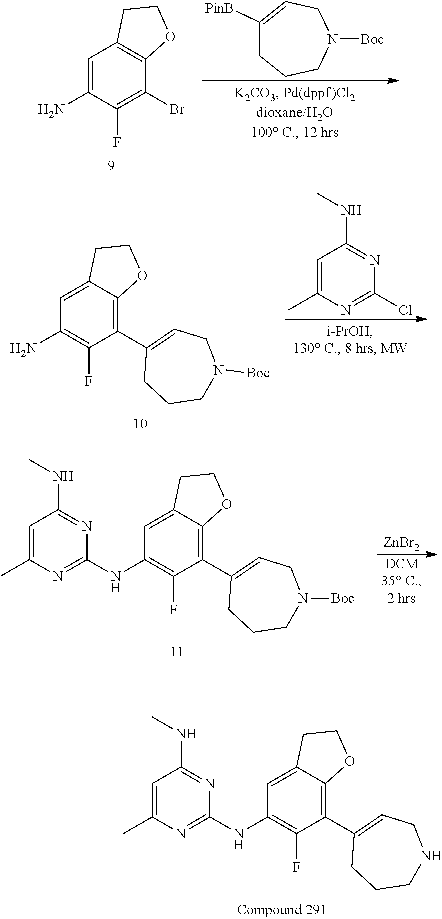

### Step 1. 2-fluoro-4-hydroxy-5-iodo-benzoic acid

K₂CO₃ (53.12 g, 384.34 mmol) was dissolved in NH₃·H₂O (300 mL), 12 (32.52 g, 128.11 mmol) was added, then the mixture was stirred at about 25° C. for about 1 hr. Then 2-fluoro-4-hydroxy-benzoic acid (20 g, 128.11 mmol) was added to the mixture was stirred at about 25° C. for about 2 hrs. LCMS showed the reaction was complete mostly and the desired mass was detected. The reaction mixture was acidified by HCl (2 M) till pH=1. The reaction mixture was added to water (200 mL), extracted with EtOAc (500 mL*3). The combined organic layers were washed with 500 mL of brine, dried over Na₂SO₄, filtered and concentrated under reduced pressure to give 2-fluoro-4-hydroxy-5-iodo-benzoic acid (36 g, crude).

¹H NMR (400 MHz, METHANOL-d₄) δ=8.25 (d, J=8.3 Hz, 1H), 6.61 (d, J=12.3 Hz, 1H)

### Step 2. 2-fluoro-4-hydroxy-5-iodo-benzoate

To a mixture of 2-fluoro-4-hydroxy-5-iodo-benzoic acid (36 g, 127.66 mmol) in MeOH (400 mL) was added SOCl₂ (20 mL) at about 0° C., then the mixture was stirred at about 70° C. for about 2 hrs. LCMS showed the reaction was complete mostly and the desired mass was detected. The reaction mixture was added to water (300 mL), extracted with EtOAc (500 mL*3). The combined organic layers were washed with 800 mL of brine, dried over Na₂SO₄, filtered and concentrated under reduced pressure to give methyl 2-fluoro-4-hydroxy-5-iodo-benzoate (33.6 g, crude).

### Step 3. methyl 2-fluoro-4-hydroxy-5-(2-trimethylsilylethynyl)benzoate

A mixture of methyl 2-fluoro-4-hydroxy-5-iodo-benzoate (5 g, 16.89 mmol), ethynyl(trimethyl)silane (8.29 g, 84.45 mmol, 11.93 mL), TEA (10.25 g, 101.34 mmol, 14.12 mL), palladium;triphenylphosphane (1.95 g, 1.69 mmol), CuI (1.13 g, 5.91 mmol, 200.33 μL) was added to solvent DMF (49.86 mL). The suspension was degassed and purged with N2 for 3 times. The mixture was stirred under N2 at about 50° C. for about 12 hrs. TLC (petroleum ether:ethyl acetate=5:1, Rf=0.7) indicated starting material was consumed completely, and major new spots were detected. The mixture was concentrated in vacuo. The residue was purified by flash chromatography (silica gel, petroleum ether/ethyl acetate=50/1 to 10/1 to 1/1). methyl 2-fluoro-4-hydroxy-5-(2-trimethylsilylethynyl)benzoate (2 g, 7.51 mmol) was obtained.

### Step 4. 6-fluorobenzofuran-5-carboxylic acid

To a mixture of methyl 2-fluoro-4-hydroxy-5-(2-trimethylsilylethynyl)benzoate (2 g, 7.51 mmol) in MeOH (20 mL) was added DIEA (1.94 g, 15.02 mmol, 2.62 mL), CuI (143.01 mg, 750.92 μmol, 25.45 μL) at about 25° C., the mixture was stirred at about 60° C. for about 2 hrs. Then K₂CO₃ (2.08 g, 15.02 mmol) was added to the mixture stirred at about 60° C. for about 12 hrs. LCMS showed the reaction was complete and the desired mass was detected. It was concentrated under reduced pressure, and then diluted by 50 mL H₂O, then added dropwise conc. HCl to pH=1. The suspension was extracted with EtOAc (10 mL*5), and then the organic phase was concentrated in vacuum. Compound 6-fluorobenzofuran-5-carboxylic acid (1.6 g, crude) was obtained.

### Step 5. 6-fluoro-2,3-dihydrobenzofuran-5-carboxylic acid

To a mixture of 6-fluorobenzofuran-5-carboxylic acid (20 g, 103.01 mmol) in MeOH (400 mL) was added Pd/C (10 g, 10% purity) under N2 at about 25° C., then the mixture was stirred at about 50° C. for about 12 hr under H₂ (15 Psi). LCMS showed the reaction was complete and the desired mass was detected. The reaction mixture was filtered and concentrated. 6-fluoro-2,3-dihydrobenzofuran-5-carboxylic acid (18 g, 91.75 mmol) was obtained.

### Step 6. 7-bromo-6-fluoro-2,3-dihydrobenzofuran-5-carboxylic acid

To a mixture of 6-fluoro-2,3-dihydrobenzofuran-5-carboxylic acid (18 g, 98.82 mmol) in AcOH (180 mL) was added dropwise Br₂ (47.38 g, 296.46 mmol, 15 mL) at about 0° C., then the mixture was stirred at about 25° C. for about 12 hrs. LCMS showed the reaction was complete and desired mass was detected. The mixture was concentrated in vacuum at about 45° C. 7-bromo-6-fluoro-2,3-dihydrobenzofuran-5-carboxylic acid (25 g, crude) was obtained.

### Step 7. benzyl N-(7-bromo-6-fluoro-2,3-dihydrobenzofuran-5-yl)carbamate

To a solution of 7-bromo-6-fluoro-2,3-dihydrobenzofuran-5-carboxylic acid (25 g, 95.77 mmol) in toluene (250 mL) was added DPPA (27.95 g, 114.92 mmol), TEA (29.07 g, 287.31 mmol, 40.04 mL). It was stirred at about 20° C. for about 1 h. Then to it was added BnOH (12.89 g, 287.31 mmol). It was stirred at about 80° C. for about 12 hrs. TLC (petroleum ether:ethyl acetate=3:1, Rf=0.5) indicated starting material was consumed completely, and one major new spot was detected. The reaction mixture was concentrated to give the crude product. The crude product was purified by column chromatography on silica gel (Petroleum ether:Ethyl acetate=1:0, 3:1, 0/1). benzyl N-(7-bromo-6-fluoro-2,3-dihydrobenzofuran-5-yl)carbamate (33 g, 90.12 mmol, 94.10% yield) was obtained.

### Step 8. 7-bromo-6-fluoro-2,3-dihydrobenzofuran-5-amine

To a mixture of benzyl N-(7-bromo-6-fluoro-2,3-dihydrobenzofuran-5-yl)carbamate (31 g, 84.66 mmol) in AcOH (100 mL) was added HBr (100 mL) at about 25° C., then the mixture was stirred at about 25° C. for about 3 hrs. LCMS showed starting material was consumed completely and desired compound was detected. The reaction was filtered, and then the filter cake was washed by EtOAc (10 ml*2), and then dissolved in H₂O (100 mL), and then to the solution was added sat. aq. Na₂CO₃ to pH=7, and then extracted with EtOAc (40 mL*3), and then the organic phase was concentrated in vacuum. 7-bromo-6-fluoro-2,3-dihydrobenzofuran-5-amine (13.5 g, 58.18 mmol) was obtained.

### Step 9. tert-butyl 5-(5-amino-6-fluoro-2,3-dihydrobenzofuran-7-yl)-2,3,4,7-tetrahydroazepine-1-carboxylate

To a solution of 7-bromo-6-fluoro-2,3-dihydrobenzofuran-5-amine (4.31 g, 18.56 mmol) in dioxane (50 mL) and H₂O (5 mL) was added tert-butyl 5-(4,4,5,5-tetramethyl-1,3,2-dioxaborolan-2-yl)-2,3,4,7-tetrahydroazepine-1-carboxylate (6 g, 18.56 mmol) and K₂CO₃ (7.70 g, 55.69 mmol), then it was added cyclopentyl(diphenyl)phosphane;dichloropalladium;iron (1.36 g, 1.86 mmol) under N₂ atmosphere. It was stirred at about 100° C. for about 12 h under N2 atmosphere. LCMS showed the reaction was complete and the desired mass was detected. The reaction was concentrated under reduced pressure to give a residue. The residue was purified by column chromatography (SiO₂, Petroleum ether/Ethyl acetate=1/0 to 2/1). Compound tert-butyl 5-(5-amino-6-fluoro-2,3-dihydrobenzofuran-7-yl)-2,3,4,7-tetrahydroazepine-1-carboxylate (5.5 g, crude) was obtained.

### Step 10. tert-butyl 5-[6-fluoro-5-[[4-methyl-6-(methylamino)pyrimidin-2-yl amino 1-2,3-dihydrobenzofuran-7-yl]-2,3,4,7-tetrahydroazepine-1-carboxylate

A solution of tert-butyl 5-(5-amino-6-fluoro-2,3-dihydrobenzofuran-7-yl)-2,3,4,7-tetrahydroazepine-1-carboxylate (7 g, 20.09 mmol) and 2-chloro-N,6-dimethyl-pyrimidin-4-amine (3.17 g, 20.09 mmol) in i-PrOH (50 mL) was stirred at about 130° C. for about 8 h. LCMS showed the reaction was complete and the desired mass was detected. The reaction was concentrated under reduced pressure to give tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (9.5 g, crude).

Step]]. N2-[6-fluoro-7-(2,3,4,7-tetrahydro-1H-azepin-5-yl]-2,3-dihydrobenzofuran-5-yl]-N4,6-dimethyl-pyrimidine-2,4-diamine

A solution of tert-butyl 5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (9.5 g, 20.23 mmol) and dibromozinc (13.67 g, 60.70 mmol) in DCM (100 mL) stirred at about 35° C. for about 2 hrs. LCMS showed the reaction was complete and the desired mass was detected. The reaction was filtered and concentrated under reduced pressure to give a residue. The crude product was purified by prep-HPLC (TFA condition:column:Phenomenex luna C18 (250*70 mm, 15 um); mobile phase:water(TFA)-ACN; B %: 3%-30%,23 min) to give a solution. The solution was basified by NH₄OH (25%) till pH=12, extracted with EtOAc (4 mL*4). The organic layer was dried over Na₂SO₄, concentrated to give the compound. Compound N2-[6-fluoro-7-(2,3,4,7-tetrahydro-1H-azepin-5-yl)-2,3-dihydrobenzofuran-5-yl]-N4,6-dimethyl-pyrimidine-2,4-diamine (6.5 g, 17.59 mmol) was obtained.

¹H NMR (400 MHz, METHANOL-d₄) δ: 7.89-7.78 (m, 1H), 6.02-5.95 (m, 1H), 5.84-5.74 (m, 1H), 4.61-4.50 (m, 2H), 3.56-3.47 (m, 2H), 3.23-3.10 (m, 4H), 2.92-2.80 (m, 3H), 2.66-2.57 (m, 2H), 2.21-2.09 (m, 3H), 1.93-1.84 (m, 2H)

MS (ESI): m/z=370.1 [M+H]

### EXAMPLE 57. Synthesis of Compound 159 and Compound 159-a

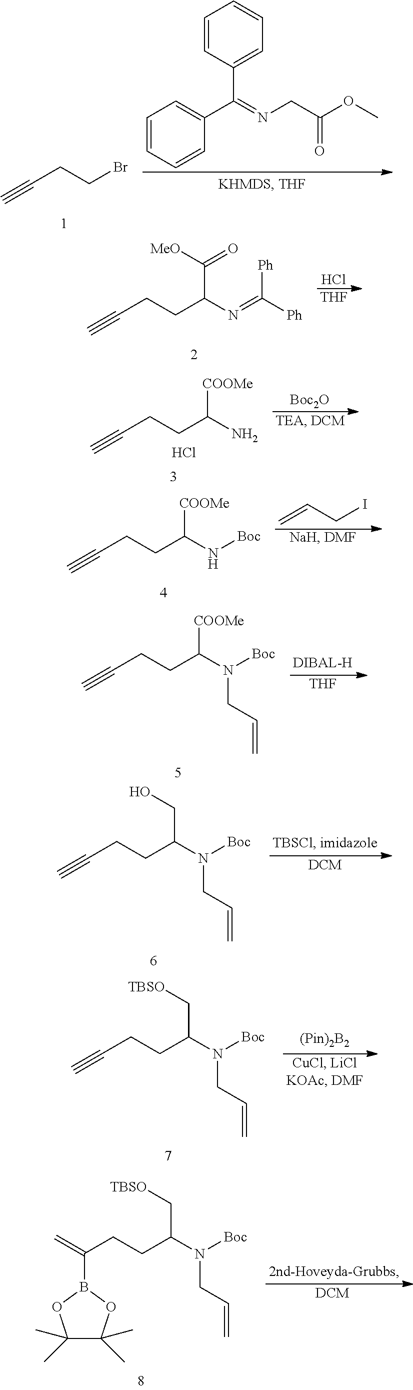

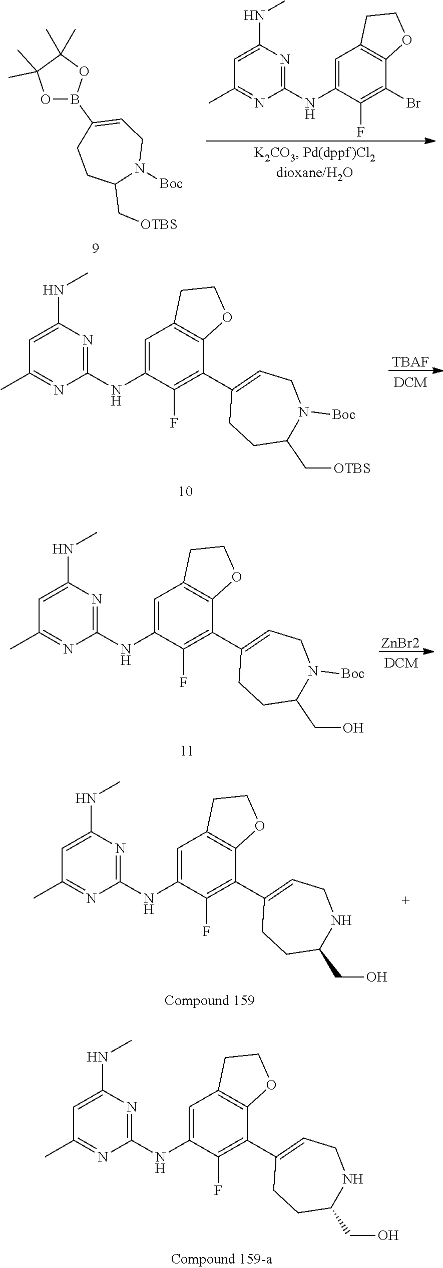

### Step 1. methyl 2-(benzhydrylideneamino)hex-5-ynoate

To a solution of 4-bromobut-1-yne (10 g, 75.20 mmol, 7.06 mL) and methyl 2-(benzhydrylideneamino)acetate (12.70 g, 50.13 mmol) in Tol (1.30 L) was added lithium;bis(trimethylsilyl)azanide (1 M, 75.20 mL), then the mixture was stirred at 100° C. for 12 h under N2. The 5 batches were combined to work up. The reaction mixture was quenched by NH₄Cl (1500 mL), extracted with EtOAc (500 mL*3). The organic layer was dried over Na₂SO₄, concentrated to give the crude product. The residue was purified by column chromatography on silica gel to give methyl 2-(benzhydrylideneamino)hex-5-ynoate (15 g, crude) as a yellow oil.

### Step 2. methyl 2-aminohex-5-ynoate

To a solution of methyl 2-(benzhydrylideneamino)hex-5-ynoate (15 g, 49.12 mmol) in THF (200 mL) was added HCl (1 M, 140 mL), then the mixture was stirred at 20° C. for 12 h. The residue was added to water/EtOAc (400 mL, v:v=1:1) and then extracted with EtOAc (150 mL*3). The water layer was alkaline by NH₄OH till pH=10, extracted with EtOAc (200 mL*3). The organic layer was dried over Na₂SO₄, concentrated to give the methyl 2-aminohex-5-ynoate (4.3 g, crude) as brown oil.

### Step 3. methyl 2-(tert-butoxycarbonylamino)hex-5-ynoate

To a solution of methyl 2-aminohex-5-ynoate (4.2 g, 29.75 mmol) in DCM (46.59 mL) was added TEA (6.02 g, 59.50 mmol, 8.29 mL) and tert-butoxycarbonyl tert-butyl carbonate (11.04 g, 50.58 mmol, 11.61 mL), then the mixture was stirred at 20° C. for 12 h. The reaction was concentrated under reduced pressure to give a residue. The residue was purified by column chromatography on silica gel to give methyl 2-(tert-butoxycarbonylamino)hex-5-ynoate (5 g, crude) as a yellow oil.

### Step 4. methyl 2-(tert-butoxycarbonylamino)hex-5-ynoate

To a solution of methyl 2-(tert-butoxycarbonylamino)hex-5-ynoate (2 g, 8.29 mmol) in DMF (50 mL) was added NaH (1.14 g, 9.53 mmol, 1.58 mL, 20% purity) at 0° C., then the mixture was stirred at 0° C. for 0.5 hr. To the mixture was added 3-iodoprop-1-ene (1.39 g, 8.29 mmol, 757.95 μL), then the mixture was stirred at 20° C. for 2 hrs. The reaction mixture was quenched by NH₄Cl (100 mL), extracted with EtOAc (100 mL*5). The organic layer was dried over Na₂SO₄, concentrated to give the crude product.

The residue was purified by column chromatography on silica gel to give methyl 2-(tert-butoxycarbonylamino)hex-5-ynoate (1.5 g, crude) as brown oil.

### Step 5. methyl 2-[allyl(tert-butoxycarbonyl)amino]hex-5-ynoate

To a solution of methyl 2-[allyl(tert-butoxycarbonyl)amino]hex-5-ynoate (5.4 g, 19.19 mmol) in THF (1.21 mL) was added DIBAL-H (1 M, 57.58 mL) at 0° C., then the mixture was stirred at 20° C. for 2 h. The reaction was concentrated under reduced pressure to give a residue. The residue was purified by column chromatography on silica gel. tert-butyl N-allyl-N-[1-(hydroxymethyl)pent-4-ynyl]carbamate (3.3 g, crude) was obtained as a yellow oil.

¹H NMR (400 MHz, CHLOROFORM-d) δ:6.00-5.71 (m, 1H), 5.26-5.01 (m, 2H), 4.13-4.11 (m, 1H), 3.98-3.57 (m, 5H), 3.48-3.30 (m, 1H), 2.30-2.11 (m, 2H), 1.86 (br s, 3H), 1.47-1.40 (m, 9H)

### Step 6. tert-butyl N-allyl-N-[1-[[tert-butyl(dimethyl)silyl]oxymethyl]pent-4-ynyl]carbamate

To the solution of tert-butyl N-allyl-N-[1-(hydroxymethyl)pent-4-ynyl]carbamate (3.2 g, 12.63 mmol) and tert-butyl-chloro-dimethyl-silane (2.87 g, 19.03 mmol, 3.54 mL) in DCM (50 mL) was added imidazole (1.38 g, 20.21 mmol), then stirred at 25° C. for 12 hrs. The reaction mixture was added to water (10 mL), extracted with DCM (20 mL*5). The organic layer was dried over Na₂SO₄, concentrated to give the crude product. The residue was purified by column chromatography on silica gel to give tert-butyl N-allyl-N-[1-[[tert-butyl(dimethyl)silyl]oxymethyl]pent-4-ynyl]carbamate (3.7 g, crude) as colorless oil.

### Step 7. tert-butyl N-allyl-N-[1-[[tert-butyl(dimethyl)silyl]oxymethyl]-4-(4,4,5,5-tetramethyl-1,3,2-dioxaborolan-2-yl)pent-4-enyl]carbamate

A mixture of LiCl (372.40 mg, 8.79 mmol, 180.08 μL) and CuCl (869.74 mg, 8.79 mmol) in DMF (20 mL) was stirred at 25° C. for 1 hrs under N2, then the solution was added tert-butyl N-allyl-N-[1-[[tert-butyl(dimethyl)silyl]oxymethyl]pent-4-ynyl]carbamate (2.7 g, 7.35 mmol), 4,4,5,5-tetramethyl-2-(4,4,5,5-tetramethyl-1,3,2-dioxaborolan-2-yl)-1,3,2-dioxaborolane (2.23 g, 8.79 mmol) and KOAc (2.49 g, 8.81 mmol) in DMF (10 mL) and stirred at 25° C. for 12 hrs under N2. The reaction was filtered and added to NH₄Cl (20 mL), extracted with EtOAc (20 mL*5). The organic layer was dried over Na₂SO₄, concentrated to give the crude product. The residue was purified by column chromatography on silica gel. tert-butyl N-allyl-N-[1-[[tert-butyl(dimethyl)silyl]oxymethyl]-4-(4,4,5,5-tetramethyl-1,3,2-dioxaborolan-2-yl)pent-4-enyl]carbamate (3.4 g, 6.86 mmol, 93.41% yield) was obtained as clear oil.

### Step 8. tert-butyl 2-[[tert-butyl(dimethyl)silyl]oxymethyl]-5-(4,4,5,5-tetramethyl-1,3,2-dioxaborolan-2-yl)-2,3,4,7-tetrahydroazepine-1-carboxylate

A solution of tert-butyl N-allyl-N-[1-[[tert-butyl(dimethyl)silyl]oxymethyl]-4-(4,4,5,5-tetramethyl-1,3,2-dioxaborolan-2-yl)pent-4-enyl]carbamate (1 g, 2.02 mmol) in DCM (10 mL) was added Hoveyda-Grubbs Catalyst 2nd Generation (60.69 mg, 96.86 mol), then stirred at 30° C. for 12 hrs under N2. The residue was purified by column chromatography on silica gel to give tert-butyl 2-[[tert-butyl(dimethyl)silyl]oxymethyl]-5-(4,4,5,5-tetramethyl-1,3,2-dioxaborolan-2-yl)-2,3,4,7-tetrahydroazepine-1-carboxylate (549 mg, crude) as clear oil.

¹H NMR (400 MHz, CHLOROFORM-d) δ:6.52-6.40 (m, 1H), 4.45-4.28 (m, 1H), 4.25-4.00 (m, 2H), 3.78-3.59 (m, 2H), 2.56-2.37 (m, 2H), 1.97-1.77 (m, 2H), 1.44 (d, J=11.2 Hz, 9H), 1.29-1.24 (m, 16H), 0.90-0.85 (m, 15H), 0.10-0.01 (m, 9H)

### Step 9. tert-butyl 2-[[tert-butyl(dimethyl)silyl]oxymethyl]-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate

To the mixture of tert-butyl 2-[[tert-butyl(dimethyl)silyl]oxymethyl]-5-(4,4,5,5-tetramethyl-1,3,2-dioxaborolan-2-yl)-2,3,4,7-tetrahydroazepine-1-carboxylate (440 mg, 941.14 μmol) and N2-(7-bromo-6-fluoro-2,3-dihydrobenzofuran-5-yl)-N4,6-dimethyl-pyrimidine-2,4-diamine (332.40 mg, 941.14 μmol) in dioxane (10 mL) and H₂O (1 mL) was added K₂CO₃ (390.21 mg, 2.82 mmol) and Pd(dppf)Cl₂ (137.73 mg, 188.23 μmol), then the reaction mixture was stirred at 100° C. for 12 hrs. The reaction was concentrated under reduced pressure to give a residue. The residue was purified by prep-TLC to give tert-butyl 2-[[tert-butyl(dimethyl)silyl]oxymethyl]-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (230 mg, crude) as brown oil.

### Step 10. N2-(7-bromo-6-fluoro-2,3-dihydrobenzofuran-5-yl)-N4,6-dimethyl-pyrimidine-2,4-diamine

To the mixture of 7-bromo-6-fluoro-2,3-dihydrobenzofuran-5-amine (250 mg, 1.08 mmol),2-chloro-N,6-dimethyl-pyrimidin-4-amine (203.75 mg, 1.29 mmol) in i-PrOH (10 mL) and TFA (0.1 mL), then stirred at 130° C. for 2 hrs. The six reactions was work up together. The reaction was concentrated under reduced pressure to give N2-(7-bromo-6-fluoro-2,3-dihydrobenzofuran-5-yl)-N4,6-dimethyl-pyrimidine-2,4-diamine (2.8 g, crude) as brown solid.

### Step 11. [rel-(2R)-5-[6-fluoro-5-[4-methyl-6-(methylamino)pyrimidin-2-yl]amino]-2,3-dihydrobenzofuran-7-yl]-2,3,4,7-tetrahydro-1H-azepin-2-yl]methanol and [rel-(2S)-5-[6-fluoro-5-[[4-methyl-6-(methylamino)pyrimidin-2-yl]amino]-2,3-dihydrobenzofuran-7-yl]-2,3,4,7-tetrahydro-1H-azepin-2-yl]methanol

To the solution of tert-butyl 5-[6-fluoro-5-[[4-methyl-6-(methylamino)pyrimidin-2-yl]amino]-2,3-dihydrobenzofuran-7-yl]-2-(hydroxymethyl)-2,3,4,7-tetrahydroazepine-1-carboxylate (100 mg, 200.17 μmol) in DCM (5 mL) was added ZnBr₂ (225.39 mg, 1.00 mmol), then stirred at 25° C. for 2 hrs. The reaction was filtered and concentrated under reduced pressure to give a residue. The crude product was purified by prep-HPLC(TFA condition:column:Phenomenex Luna 80*30 mm*3 um; mobile phase:water(TFA)-ACN; B %: 10%-40%,8 min) to give a residue. The residue was separated by SFC (column: DAICEL CHIRALPAK AD(250 mm*30 mm, 10 um).; mobile phase: A: CO2 B:MeOH(0.1% IPAm, v/v; SFC, Acq Method: C2_MeOH_IPAm_50_4_35) to give 7.5 mg P1 (Rt=1.810 min) (SFC, Method:C2_MeOH_IPAm_50_4_35)
and 8.6 mg P2 (Rt=2.540 min) (SFC, Method: C2_MeOH_IPAm_50_4_35).

[rel-(2R)-5-[6-fluoro-5-[[4-methyl-6-(methylamino)pyrimidin-2-yl]amino]-2,3-dihydrobenzofuran-7-yl]-2,3,4,7-tetrahydro-1H-azepin-2-yl]methanol (3.4 mg, 8.51 μmol, 4.25% yield) (de %=100%) was obtained as pale yellow solid.

[rel-(2S)-5-[6-fluoro-5-[[4-methyl-6-(methylamino)pyrimidin-2-yl]amino]-2,3-dihydrobenzofuran-7-yl]-2,3,4,7-tetrahydro-1H-azepin-2-yl]methanol (3.1 mg, 7.76 μmol, 3.88% yield)(de %=100%) was obtained as a pale yellow solid.

¹H NMR (400 MHz, METHANOL-d₄) δ: 7.94-7.76 (m, 1H), 6.08-5.93 (m, 1H), 5.88-5.74 (m, 1H), 4.59-4.44 (m, 2H), 3.66-3.52 (m, 2H), 3.50-3.41 (m, 2H), 3.18 (br t, J=8.6 Hz, 2H), 3.04-2.93 (m, 1H), 2.85 (s, 3H), 2.68-2.50 (m, 2H), 2.16 (s, 3H), 1.93-1.77 (m, 1H), 1.70-1.55 (m, 1H)

¹H NMR (400 MHz, METHANOL-d₄) δ: 7.84 (br s, 1H), 6.01 (br t, J=5.2 Hz, 1H), 5.79 (s, 1H), 4.59-4.50 (m, 2H), 3.66-3.52 (m, 2H), 3.50-3.39 (m, 2H), 3.23-3.13 (m, 2H), 3.05-2.92 (m, 1H), 2.90-2.80 (m, 3H), 2.69-2.50 (m, 2H), 2.16 (s, 3H), 1.93-1.81 (m, 1H), 1.68-1.53 (m, 1H)

### EXAMPLE 58. Synthesis of Compound 160, Compound 356 and Compound 356-a

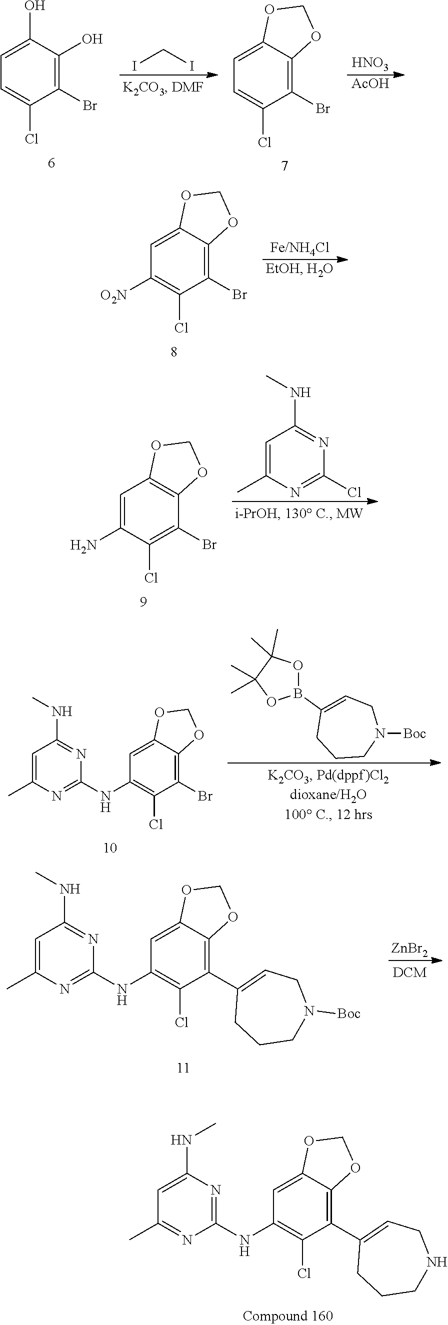

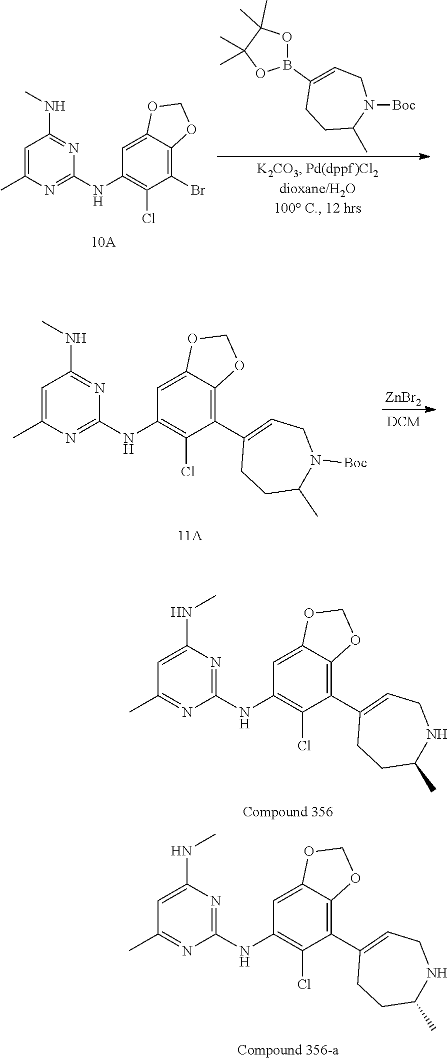

### Step 1. 4-bromo-5-chloro-1,3-benzodioxole

To a mixture of 3-bromo-4-chloro-benzene-1,2-diol (500 mg, 2.24 mmol) in DMF (5 mL) was added C_{S2}CO₃ (2.19 g, 6.71 mmol), KF (130.01 mg, 2.24 mmol), diiodomethane (1.20 g, 4.48 mmol, 360.49 μL) at 25° C., then the mixture was stirred at 100° C. for 12 h. The reaction mixture was poured into water (30 mL), extracted with EtOAc (30 mL*3). The combined organic layers were washed with 30 mL of brine, dried over anhydrous Na₂SO₄, filtered and concentrated under reduced pressure to give a residue. The residue was purified by column chromatography. 4-bromo-5-chloro-1,3-benzodioxole (450 mg, 1.91 mmol, 85.41% yield) was obtained as a yellow solid.

¹H NMR (400 MHz, CHLOROFORM-d) δ:8.00 (s, 1H), 6.94 (d, J=8.3 Hz, 1H), 6.69 (d, J=8.3 Hz, 1H), 6.06 (s, 2H), 2.95 (s, 3H), 2.87 (s, 3H).

### Step 2. tert-butyl5-(6-chloro-7-((4-methyl-6-(methylamino)pyrimidin-2-yl)amino)-2,3-dihydrobenzo[b][1,4dioxin-5-yl)-2-methyl-2,3,4,7-tetrahydro-1H-azepine-1-carboxylate

To a solution of 4-bromo-5-chloro-1,3-benzodioxole (350 mg, 1.49 mmol) in AcOH (7 mL) was added HNO₃ (2.81 g, 44.59 mmol, 1.87 mL) at 0° C., then the mixture was stirred at 25° C. for 3 h. The reaction mixture was added to ice water (30 mL), the mixture was filtered and the filter liquid was dried in vacuum to give the crude product which was purified by silica gel column chromatography to give 4-bromo-5-chloro-6-nitro-1,3-benzodioxole (540 mg, crude) as a light-yellow solid.

¹H NMR (400 MHz, CHLOROFORM-d) δ:7.41-7.33 (m, 1H), 6.26-6.17 (m, 2H).

### Step 3. 7-bromo-6-chloro-],3-benzodioxol-5-amine

To a solution of 4-bromo-5-chloro-6-nitro-1,3-benzodioxole (540 mg, 1.93 mmol) in H₂O (6 mL) and EtOH (30 mL), Fe (537.62 mg, 9.63 mmol), NH₄Cl (1.03 g, 19.25 mmol) was added, then the mixture was stirred at 80° C. for 2 hr. The reaction mixture was filtered through a pad of celite and the filter liquid was dried in vacuum to 7-bromo-6-chloro-1,3-benzodioxol-5-amine (380 mg, 1.52 mmol, 78.79% yield).

### Step 4. N2-(7-bromo-6-chloro-1,3-benzodioxol-5-yl)-N4,6-dimethyl-pyrimidine-2,4-diamine

To a solution of 7-bromo-6-chloro-1,3-benzodioxol-5-amine (410 mg, 1.64 mmol) and 2-chloro-N,6-dimethyl-pyrimidin-4-amine (257.97 mg, 1.64 mmol) in i-PrOH (10 mL) was added TFA (350.89 mg, 3.08 mmol, 237.09 μL), then the mixture was stirred at 130° C. for 2 h under M. W. condition. The reaction was concentrated under reduced pressure to give N2-(7-bromo-6-chloro-1,3-benzodioxol-5-yl)-N4,6-dimethyl-pyrimidine-2,4-diamine (250 mg, 672.74 μmol, 41.10% yield) as a brown solid.

### Step 5. tert-butyl 5-[5-chloro-6-[[4-methyl-6-(methylamino)pyrimidin-2-yl]amino]-1,3-benzodioxol-4-yl]-2,3,4,7-tetrahydroazepine-1-carboxylate

To a mixture of N2-(7-bromo-6-chloro-1,3-benzodioxol-5-yl)-N4,6-dimethyl-pyrimidine-2,4-diamine (40 mg, 107.64 μmol), tert-butyl 5-(4,4,5,5-tetramethyl-1,3,2-dioxaborolan-2-yl)-2,3,4,7-tetrahydroazepine-1-carboxylate (34.79 mg, 107.64 μmol) in H₂O (0.1 mL) and dioxane (1 mL) was added Pd(dppf)Cl₂ (8.79 mg, 10.76 μmol) and K₂CO₃ (29.75 mg, 215.28 mol) under N₂ atmosphere. It was stirred at 100° C. for 12 h under N₂ atmosphere. The reaction mixture was filtered and concentrated under reduced pressure to give the crude product which was purified by prep-TLC to give tert-butyl 5-[5-chloro-6-[[4-methyl-6-(methylamino)pyrimidin-2-yl]amino]-1,3-benzodioxol-4-yl]-2,3,4,7-tetrahydroazepine-1-carboxylate (60 mg, 122.96 mol) as a yellow solid.

### Step 6. N2-[6-chloro-7-(2,3,4,7-tetrahydro-1H-azepin-5-yl)-1,3-benzodioxol-5-yl]-N4,6-dimethyl-pyrimidine-2,4-diamine

To a solution of tert-butyl 5-[5-chloro-6-[[4-methyl-6-(methylamino)pyrimidin-2-yl]amino]-1,3-benzodioxol-4-yl]-2,3,4,7-tetrahydroazepine-1-carboxylate (60 mg, 122.96 μmol) in DCM (2 mL) was added ZnBr₂ (138.45 mg, 614.78 mol), then the mixture was stirred at 30° C. for 3 hr. The reaction was filtered and concentrated under reduced pressure to give the crude product which was purified by prep-HPLC(TFA condition:column:Phenomenex Luna 80*30 mm*3 um; mobile phase:water(TFA)-ACN; B %: 1%-30%,8 min) to give N2-[6-chloro-7-(2,3,4,7-tetrahydro-1H-azepin-5-yl)-1,3-benzodioxol-5-yl]-N4,6-dimethyl-pyrimidine-2,4-diamine (22 mg, 43.92 μmol, 35.72% yield, TFA) as a pale yellow solid.

¹H NMR (400 MHz, METHANOL-d₄) δ: 7.23 (s, 1H), 6.10-6.03 (m, 2H), 6.01-5.92 (m, 2H), 4.00-3.82 (m, 2H), 3.55-3.41 (m, 2H), 2.96-2.85 (m, 3H), 2.77-2.64 (m, 2H), 2.33-2.20 (m, 3H), 2.17-2.04 (m, 2H).

### Step 7. tert-butyl5-[5-chloro-6-[[4-methyl-6-(methylamino)pyrimidin-2-ylamino-1,3-benzodioxol-4-yl]-2-methyl-2,3,4,7-tetrahydroazepine-1-carboxylate

To a mixture of N2-(7-bromo-6-chloro-1,3-benzodioxol-5-yl)-N4,6-dimethyl-pyrimidine-2,4-diamine (80 mg, 215.28 μmol), tert-butyl 2-methyl-5-(4,4,5,5-tetramethyl-1,3,2-dioxaborolan-2-yl)-2,3,4,7-tetrahydroazepine-1-carboxylate (72.60 mg, 215.28 μmol) in H₂O (0.2 mL) and dioxane (2 mL) was addedcyclopentyl(diphenyl)phosphane;dichloromethane;dichloropalladium;iron (17.58 mg, 21.53 μmol) and K₂CO₃ (59.50 mg, 430.55 mol) under N₂ atmosphere. It was stirred at 100° C. for 12 h under N₂ atmosphere. The reaction mixture was filtered and concentrated under reduced pressure to give the crude product. tert-butyl 5-[5-chloro-6-[[4-methyl-6-(methylamino)pyrimidin-2-yl]amino]-1,3-benzodioxol-4-yl]-2-methyl-2,3,4,7-tetrahydroazepine-1-carboxylate (120 mg, 239.04 μmol) was obtained as a black solid.

### Step 8. N2-[6-chloro-7-[rel-(2S)-2-methyl-2,3,4,7-tetrahydro-1H-azepin-5-yl]-1,3-benzodioxol-5-yl]-N4,6-dimethyl-pyrimidine-2,4-diamine and N2-[6-chloro-7-[rel-(2R)-2-methyl-2,3,4,7-tetrahydro-1H-azepin-5-yl]-1,3-benzodioxol-5-yl-N4,6-dimethyl-pyrimidine-2,4-diamine

To a solution of tert-butyl 5-[5-chloro-6-[[4-methyl-6-(methylamino)pyrimidin-2-yl]amino]-1,3-benzodioxol-4-yl]-2-methyl-2,3,4,7-tetrahydroazepine-1-carboxylate (120 mg, 239.04 μmol) in DCM (4 mL) was added ZnBr₂ (269.16 mg, 1.20 mmol), then the mixture was stirred at 30° C. for 3 hr. LCMS showed the starting materials was consumed completely and the desired MS was detected. The reaction was concentrated under reduced pressure to give a residue. The residue was purified by Prep-HPLC (TFA condition, column: Phenomenex Luna 80*30 mm*3 um; mobile phase: [water(TFA)-ACN; B %: 1%-30%, 8 min) to give crude product. The crude product was purified by SFC (basic condition, column: Column: Chiralpak IG-3, 100×4.6 mm I.D., 3 um); mobile phase: A: CO₂ B: IPA (0.1% IPAm) to give N2-[6-chloro-7-[rel-(2S)-2-methyl-2,3,4,7-tetrahydro-1H-azepin-5-yl]-1,3-benzodioxol-5-yl]-N4,6-dimethyl-pyrimidine-2,4-diamine (16 mg, 39.81 μmol, 16.65% yield) (purity: 99.781%, ee: 100%) as a pale yellow solid and N2-[6-chloro-7-[rel-(2R)-2-methyl-2,3,4,7-tetrahydro-1H-azepin-5-yl]-1,3-benzodioxol-5-yl]-N4,6-dimethyl-pyrimidine-2,4-diamine (18 mg, 44.79 μmol, 18.74% yield) (purity: 99.369%, ee: 99.08%) as a white solid.

### Product 1:

¹H NMR (400 MHz, METHANOL-d₄) δ: 8.15-7.90 (m, 1H), 5.90-5.77 (m, 1H), 5.76-5.62 (m, 1H), 5.01-4.62 (m, 16H), 4.35-4.01 (m, 4H), 3.62-3.38 (m, 2H), 3.35-3.16 (m, 9H), 3.15-3.02 (m, 1H), 2.95-2.84 (m, 3H), 2.67-2.50 (m, 1H), 2.44-2.27 (m, 1H), 2.25-2.08 (m, 3H), 2.04-1.85 (m, 1H), 1.78-1.61 (m, 1H), 1.23-1.11 (m, 3H)

### Product 2:

¹H NMR (400 MHz, METHANOL-d₄) δ: 8.13-7.92 (m, 1H), 5.89-5.76 (m, 1H), 5.74-5.59 (m, 1H), 4.32-4.11 (m, 4H), 3.64-3.40 (m, 2H), 3.19-3.05 (m, 1H), 2.99-2.76 (m, 3H), 2.67-2.50 (m, 1H), 2.44-2.24 (m, 1H), 2.23-2.09 (m, 3H), 2.05-1.83 (m, 1H), 1.81-1.58 (m, 1H), 1.26-1.05 (m, 3H).

### EXAMPLE 59. Synthesis of Compound 161 and Compound 161-a

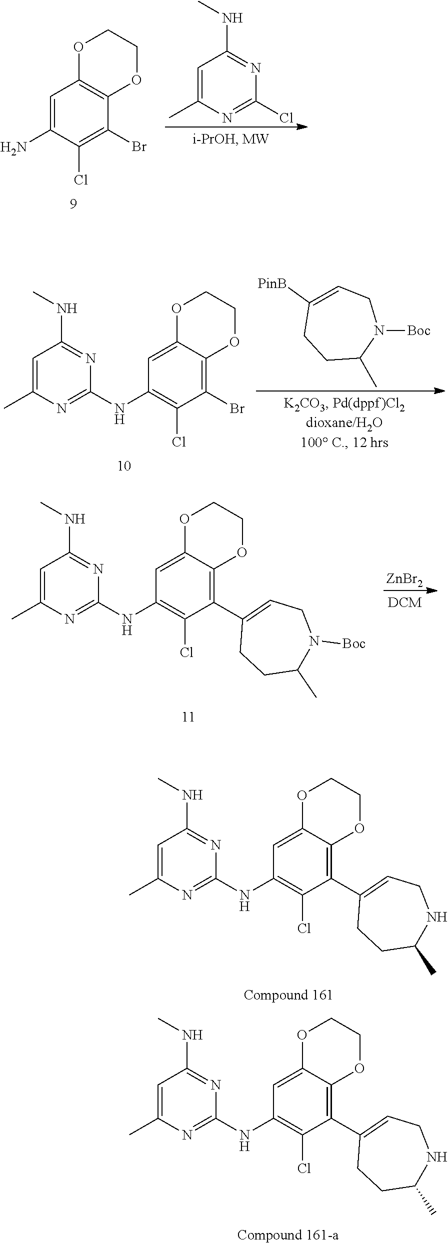

### Step 1. N2-(5-bromo-6-chloro-2,3-dihydro-1,4-benzodioxin-7-yl)-N4,6-dimethyl-pyrimidine-2,4-diamine

To a solution of 5-bromo-6-chloro-2,3-dihydro-1,4-benzodioxin-7-amine (53 mg, 200.38 mol) and 2-chloro-N,6-dimethyl-pyrimidin-4-amine (31.58 mg, 200.38 μmol) in i-PrOH, (2 mL) was added TFA (42.95 mg, 376.71 μmol, 29.02 μL), then the mixture was stirred at 140° C. for 2 hrs under M. W. condition. The reaction was concentrated under reduced pressure to give N2-(5-bromo-6-chloro-2,3-dihydro-1,4-benzodioxin-7-yl)-N4,6-dimethyl-pyrimidine-2,4-diamine (80 mg, crude) as brown solid.

### Step 2. tert-butyl 5-(6-chloro-7-((4-methyl-6-(methylamino)pyrimidin-2-yl)amino)-2,3-dihydrobenzo[b][1, 4]dioxin-5-yl)-2-methyl-2,3,4,7-tetrahydro-1H-azepine-1-carboxylate

To a mixture of N2-(5-bromo-6-chloro-2,3-dihydro-1,4-benzodioxin-7-yl)-N4,6-dimethyl-pyrimidine-2,4-diamine (40 mg, 103.72 μmol), tert-butyl 2-methyl-5-(4,4,5,5-tetramethyl-1,3,2-dioxaborolan-2-yl)-2,3,4,7-tetrahydroazepine-1-carboxylate (38.48 mg, 114.10 mol) in H₂O (0.4 mL), THF(1.6 mL) was added K₃PO₄ (66.05 mg, 311.17 μmol) and cataCxiumAPd G2 (6.94 mg, 10.37 mol) at 25° C., then the mixture was stirred at 80° C. for 4 h under N2. The reaction mixture was concentrated to give tert-butyl 5-(6-chloro-7-((4-methyl-6-(methylamino)pyrimidin-2-yl)amino)-2,3-dihydrobenzo[b][1,4]dioxin-5-yl)-2-methyl-2,3,4,7-tetrahydro-1H-azepine-1-carboxylate (100 mg, 193.79 mol) as black solid.

### Step 3. N2-[6-chloro-5-[rel-(2S)-2-methyl-2,3,4,7-tetrahydro-1H-azepin-5-yl]-2,3-dihydro-1,4-enzodioxin-7-yl]-N4,6-dimethyl-pyrimidine-2,4-diamine and N2-[6-chloro-5-[rel-(2R)-2-methyl-2, 3, 4, 7-tetrahydro-1H-azepin-5-yl]-2,3-dihydro-1,4-benzodioxin-7-yl]-N4,6-dimethyl-pyrimidine-2,4-diamine

To a solution of tert-butyl 5-[6-chloro-7-[[4-methyl-6-(methylamino)pyrimidin-2-yl]amino]-2,3-dihydro-1,4-benzodioxin-5-yl]-2-methyl-2,3,4,7-tetrahydroazepine-1-carboxylate (100 mg, 193.79 mol) in DCM (2 mL) was added ZnBr₂ (218.20 mg, 968.93 μmol), then the mixture was stirred at 30° C. for 3 h. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give crude product. The crude product was purified by SFC (basic condition, column: Chiralpak IG-3, 100×4.6 mm I.D., 3 um); mobile phase: A: C02 B: IPA (0.1% IPAm) to give N2-[6-chloro-5-[rel-(2S)-2-methyl-2,3,4,7-tetrahydro-1H-azepin-5-yl]-2,3-dihydro-1,4-benzodioxin-7-yl]-N4,6-dimethyl-pyrimidine-2,4-diamine (4.8 mg, 11.54 μmol, 5.96% yield) (purity: 93.788%, ee: 100%) as a pale yellow solid and N2-[6-chloro-5-[rel-(2R)-2-methyl-2,3,4,7-tetrahydro-1H-azepin-5-yl]-2,3-dihydro-1,4-benzodioxin-7-yl]-N4,6-dimethyl-pyrimidine-2,4-diamine (3.7 mg, 8.90 μmol, 4.59% yield) (purity: 99.125%, ee: 96.68%) as a white solid.

¹H NMR (400 MHz, METHANOL-d₄) δ: 8.15-7.90 (m, 1H), 5.90-5.77 (m, 1H), 5.76-5.62 (m, 1H), 5.01-4.62 (m, 16H), 4.35-4.01 (m, 4H), 3.62-3.38 (m, 2H), 3.35-3.16 (m, 9H), 3.15-3.02 (m, 1H), 2.95-2.84 (m, 3H), 2.67-2.50 (m, 1H), 2.44-2.27 (m, 1H), 2.25-2.08 (m, 3H), 2.04-1.85 (m, 1H), 1.78-1.61 (m, 1H), 1.23-1.11 (m, 3H)

¹H NMR (400 MHz, METHANOL-d₄) δ: 8.13-7.92 (m, 1H), 5.89-5.76 (m, 1H), 5.74-5.59 (m, 1H), 4.32-4.11 (m, 4H), 3.64-3.40 (m, 2H), 3.19-3.05 (m, 1H), 2.99-2.76 (m, 3H), 2.67-2.50 (m, 1H), 2.44-2.24 (m, 1H), 2.23-2.09 (m, 3H), 2.05-1.83 (m, 1H), 1.81-1.58 (m, 1H), 1.26-1.05 (m, 3H).

### EXAMPLE 60. Synthesis of Compound 162 and Compound 164

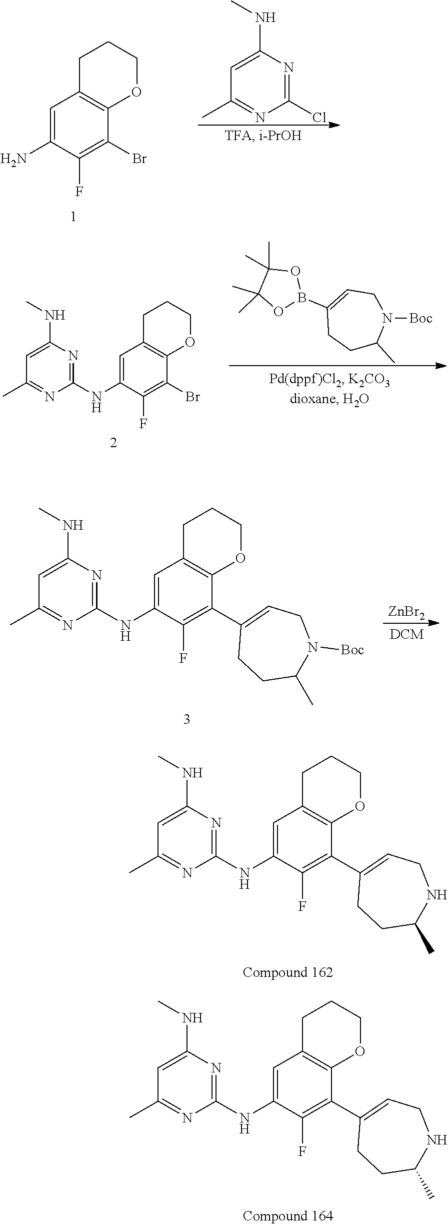

### Step 1. N2-(8-bromo-7-fluoro-chroman-6-yl)-N4,6-dimethyl-pyrimidine-2,4-diamine

To a solution of 8-bromo-7-fluoro-chroman-6-amine (100 mg, 406.38 μmol) and 2-chloro-N,6-dimethyl-pyrimidin-4-amine (64.05 mg, 406.38 μmol) in i-PrOH (2 mL) was added TFA (4.63 mg, 40.64 μmol, 3.13 μL), then the mixture was stirred at 130° C. for 3 h. The reaction was concentrated under reduced pressure to give N2-(8-bromo-7-fluoro-chroman-6-yl)-N4,6-dimethyl-pyrimidine-2,4-diamine (190 mg, crude, TFA) as a white solid.

### Step 2. tert-butyl 5-[7-fluoro-6-[[4-methyl-6-(methylamino)pyrimidin-2-yl]amino]chroman-8-yl]-2-methyl-2,3,4,7-tetrahydroazepine-1-carboxylate

To a solution of N2-(8-bromo-7-fluoro-chroman-6-yl)-N4,6-dimethyl-pyrimidine-2,4-diamine (100 mg, 208.23 μmol, TFA), tert-butyl 2-methyl-5-(4,4,5,5-tetramethyl-1,3,2-dioxaborolan-2-yl)-2,3,4,7-tetrahydroazepine-1-carboxylate (77.25 mg, 229.06 μmol) and K₂CO₃ (86.34 mg, 624.70 μmol) in dioxane (2 mL) and H₂O (0.2 mL) was added Pd(dppf)Cl₂ (15.24 mg, 20.82 μmol), then the mixture was stirred at 100° C. for 12 h under an atmosphere of nitrogen. The reaction was filtered and concentrated under reduced pressure to give tert-butyl-5-[7-fluoro-6-[[4-methyl-6-(methylamino)pyrimidin-2-yl]amino]chroman-8-yl]-2-methyl-2,3,4,7-tetrahydroazepine-1-carboxylate (100 mg, crude) as a black solid.

### Step 3. N2-[7-fluoro-8-[rel-(2S)-2-methyl-2,3,4,7-tetrahydro-1H-azepin-5-yl]chroman-6-yl-N4,6-dimethyl-pyrimidine-2,4-diamine and N2-[7-fluoro-8-[rel-(2R)-2-methyl-2,3,4,7-tetrahydro-1H-azepin-5-yl]chroman-6-yl]-N4,6-dimethyl-pyrimidine-2,4-diamine

To a solution of tert-butyl 5-[7-fluoro-6-[[4-methyl-6-(methylamino)pyrimidin-2-yl]amino]chroman-8-yl]-2-methyl-2,3,4,7-tetrahydroazepine-1-carboxylate (100 mg, 200.96 μmol) in DCM (3 mL) was added ZnBr₂ (452.57 mg, 2.01 mmol), then the mixture was stirred at 35° C. for 5 h. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give crude product. The crude product was purified by Chiral SFC (Column: DAICEL CHIRALCEL OX(250 mm*30 mm, 10 um); Mobile phase: A for CO₂ and B for MeOH(0.1% NH₃H₂O); Gradient: B %=60% isocratic elution mode; Flow rate: 80 g/min; Wavelength: 220 nm; Column temperature: 40° C.; System back pressure: 100 bar) to give N2-[7-fluoro-8-[rel-(2S)-2-methyl-2,3,4,7-tetrahydro-1H-azepin-5-yl]chroman-6-yl]-N4,6-dimethyl-pyrimidine-2,4-diamine (16.6 mg, 32.52 μmol, 16.18% yield, TFA) (purity: 100%, ee: 99.7%) as a light-yellow solid and N2-[7-fluoro-8-[rel-(2R)-2-methyl-2,3,4,7-tetrahydro-1H-azepin-5-yl]chroman-6-yl]-N4,6-dimethyl-pyrimidine-2,4-diamine (17.1 mg, 33.50 μmol, 16.67% yield, TFA) (purity: 100%, ee: 97.66%) as a light-yellow solid.

¹H NMR (400 MHz, METHANOL-d₄) δ:1.17 (d, J=6.50 Hz, 3H) 1.55-1.69 (m, 1H) 1.82-1.93 (m, 1H) 1.93-2.01 (m, 2H) 2.16 (s, 3H) 2.38 (br dd, J=15.38, 7.00 Hz, 1H) 2.51-2.64 (m, 1H)2.76 (br t, J=6.44 Hz, 2H)2.87 (s, 3H)2.99-3.10 (m, 1H)3.38-3.53 (m,2 H) 4.13 (dd, J=5.69, 4.44 Hz, 2H) 5.75-5.86 (m, 2H) 7.78 (br d, J=6.50 Hz, 1H)

¹H NMR (400 MHz, METHANOL-d₄) δ:1.16 (d, J=6.50 Hz, 3H) 1.56-1.67 (m, 1H) 1.83-1.91 (m, 1H) 1.93-2.02 (m, 2H) 2.16 (s, 3H) 2.38 (br dd, J=15.63, 7.13 Hz, 1H) 2.51-2.64 (m, 1H) 2.76 (br t, J=6.38 Hz, 2H) 2.87 (s, 3H) 2.97-3.09 (m, 1H) 3.38-3.52 (m, 2H) 4.13 (br dd, J=5.63, 4.50 Hz, 2H) 5.75-5.89 (m, 2H) 7.78 (br d, J=6.75 Hz, 1H).

### EXAMPLE 61. Synthesis of Compound 163-a and Compound 163

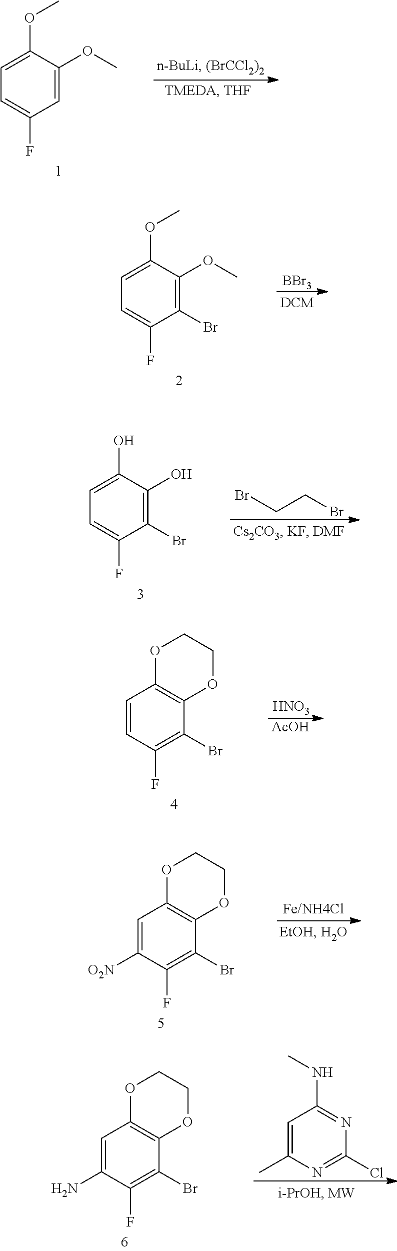

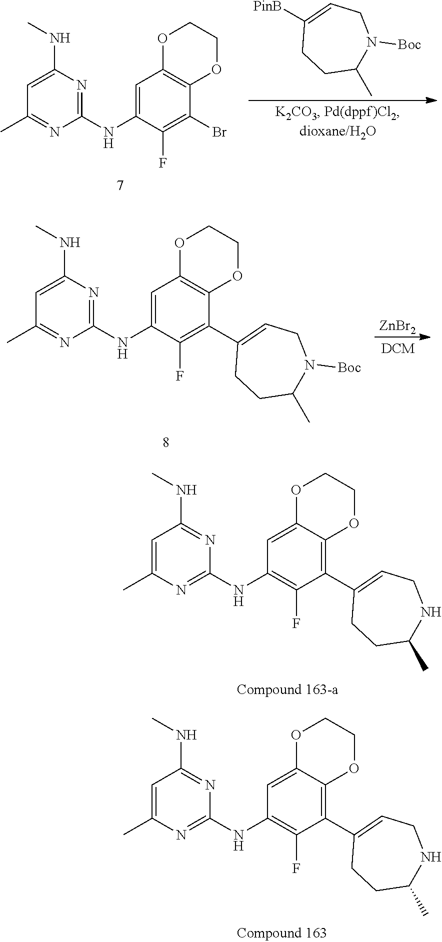

### Step 1. 2-bromo-1-fluoro-3,4-dimethoxy-benzene

n-butyllithium (2.5 M, 29.20 mL) was added dropwise at −78° C. to a solution of compound 4-fluoro-1,2-dimethoxy-benzene (7.6 g, 48.67 mmol, 6.49 mL) and N,N,N′,N′-tetramethylethane-1,2-diamine (5.66 g, 48.67 mmol, 7.30 mL) in THF (80 mL) under nitrogen atmosphere and stirred at −78° C. for 1.5 h. Then 1,2-dibromo-1,1,2,2-tetrachloro-ethane (20.76 g, 63.76 mmol, 7.65 mL) in THF (20 mL) was added, after stirring for a further 10 min, the cooling bath was removed and the reaction vessel allowed to warm to room temperature. The reaction mixture was quenched by sat. NH₄Cl aq.(100 mL), extracted with EtOAc (300 mL*3). The organic layer was dried over Na₂SO₄, concentrated to give the crude product. The crude product was purified by column chromatography on silica gel to give 2-bromo-1-fluoro-3,4-dimethoxy-benzene (8.5 g, crude) as a yellow gum.

¹H NMR (400 MHz, CHLOROFORM-d) δ:3.86 (s, 3H) 3.89 (s, 3H) 6.82-6.85 (m, 1H)6.85-6.89 (m, 1H)

### Step 2. 3-bromo-4-fluoro-benzene-1,2-diol

To a mixture of 2-bromo-1-fluoro-3,4-dimethoxy-benzene (8.5 g, 36.16 mmol) in DCM (90 mL) was added BBr₃ (45.30 g, 180.81 mmol) in DCM (90 mL) at 0° C., then the mixture was stirred at 25° C. for 3 hrs. The reaction mixture was quenched by MeOH (15 mL) at 0° C. The reaction mixture was filtered and concentrated to give 3-bromo-4-fluoro-benzene-1,2-diol (7.5 g, crude) as a brown gum.

¹H NMR (400 MHz, CHLOROFORM-d) δ:6.62-6.67 (m, 1H) 6.84 (dd, J=8.94, 5.07 Hz, 1H)

### Step 3. 5-bromo-6-fluoro-2,3-dihydro-1,4-benzodioxine

To a mixture of 3-bromo-4-fluoro-benzene-1,2-diol (7.5 g, 36.23 mmol) in DMF (100 mL) was added C_{S2}CO₃ (35.42 g, 108.70 mmol), KF (2.10 g, 36.23 mmol),1,2-dibromoethane (13.61 g, 72.46 mmol, 6.24 mL) at 25° C., then the mixture was stirred at 100° C. for 12 hrs. The reaction mixture was added to water (150 mL), extracted with EtOAc (500 mL*3). The combined organic layers were washed with 500 mL of brine, dried over Na₂SO₄, filtered and concentrated under reduced pressure to give a residue. The crude product was purified by column chromatography on silica gel to give 5-bromo-6-fluoro-2,3-dihydro-1,4-benzodioxine (7.13 g, crude) as a white solid.

¹H NMR (400 MHz, CHLOROFORM-d) δ:4.22-4.26 (m, 2H) 4.35-4.39 (m, 2H) 6.63-6.69 (m, 1H) 6.80 (dd, J=8.99, 5.26 Hz, 1H)

### Step 4. 5-bromo-6-fluoro-7-nitro-2,3-dihydro-1,4-benzodioxine

To a mixture of HNO₃ (140.00 g, 2.22 mol, 100 mL) in AcOH (350 mL) was added 5-bromo-6-fluoro-2,3-dihydro-1,4-benzodioxine (5 g, 21.46 mmol) at 0° C., then the mixture was stirred at 25° C. for 3 hrs. The reaction mixture was added to ice water (500 mL), the mixture was filtered and the filter cake was dried in vacuum to give 5-bromo-6-fluoro-7-nitro-2,3-dihydro-1,4-benzodioxine (2 g, crude) as a yellow solid.

### Step 5. 5-bromo-6-fluoro-2,3-dihydro-1,4-benzodioxin-7-amine

To a mixture of 5-bromo-6-fluoro-7-nitro-2,3-dihydro-1,4-benzodioxine (1.4 g, 5.04 mmol) in EtOH (30 mL), H₂O (3 mL) was added Fe (1.41 g, 25.18 mmol) and NH₄Cl (2.69 g, 50.35 mmol) at 25° C., then the mixture was stirred at 80° C. for 12 hrs. The reaction mixture was filtered and concentrated to give the crude product. The crude product was added to water (30 mL), extracted with EtOAc (100 mL*3). The combined organic layers were washed with 50 mL of brine, dried over Na₂SO₄, filtered and concentrated under reduced pressure to give the crude product. The crude product was purified by prep-HPLC (TFA condition; column: Phenomenex luna C18 250*50 mm*10 um; mobile phase: [water(0.1% TFA)-ACN]; B %: 30%-70%,10 min) to give 5-bromo-6-fluoro-2,3-dihydro-1,4-benzodioxin-7-amine (300 mg, crude) as a yellow solid ¹H NMR (400 MHz, CHLOROFORM-d) δ:4.22 (td, J=2.63, 1.53 Hz, 2H) 4.29 (br dd, J=3.62, 1.64 Hz, 2H) 6.33 (dd, J=8.33, 1.53 Hz, 1H)

### Step 6. N2-(5-bromo-6-fluoro-2,3-dihydro-1,4-benzodioxin-7-yl)-N4,6-dimethyl-pyrimidine-2,4-diamine

To a solution of 5-bromo-6-fluoro-2,3-dihydro-1,4-benzodioxin-7-amine (50 mg, 201.57 mol) and 2-chloro-N,6-dimethyl-pyrimidin-4-amine (31.77 mg, 201.57 μmol) in i-PrOH (1 mL) was added TFA (43.25 mg, 379.31 μmol, 29.22 μL), then the mixture was stirred at 140° C. in the microwave for 2 hrs. The reaction was concentrated under reduced pressure to give N2-(5-bromo-6-fluoro-2,3-dihydro-1,4-benzodioxin-7-yl)-N4,6-dimethyl-pyrimidine-2,4-diamine (70 mg, crude) as a brown solid.

### Step 7. tert-butyl 5-[6-fluoro-7-[[4-methyl-6-(methylamino)pyrimidin-2-ylamino-2,3-dihydro-1,4-benzodioxin-5-yl]-2-methyl-2,3,4,7-tetrahydroazepine-1-carboxylate

To a mixture of tert-butyl 2-methyl-5-(4,4,5,5-tetramethyl-1,3,2-dioxaborolan-2-yl)-2,3,4,7-tetrahydroazepine-1-carboxylate (63.95 mg, 189.61 μmol), N2-(5-bromo-6-fluoro-2,3-dihydro-1,4-benzodioxin-7-yl)-N4,6-dimethyl-pyrimidine-2,4-diamine (70 mg, 189.61 μmol) in THF (2 mL), H₂O (0.5 mL) was added K₃PO₄ (80.49 mg, 379.21 μmol) and cataCxiumAPd G2 (12.68 mg, 18.96 μmol) at 25° C., then the mixture was stirred at 80° C. for 12 hrs under N2. The reaction was concentrated under reduced pressure to give a residue. The residue was purified by prep-TLC to give tert-butyl 5-[6-fluoro-7-[[4-methyl-6-(methylamino)pyrimidin-2-yl]amino]-2,3-dihydro-1,4-benzodioxin-5-yl]-2-methyl-2,3,4,7-tetrahydroazepine-1-carboxylate (90 mg, crude) as a white solid.

### Step 8. N2-[6-fluoro-5-[rel-(2S)-2-methyl-2,3,4,7-tetrahydro-1H-azepin-5-yl]-2,3-dihydro-1,4-benzodioxin-7-yl J-N4,6-dimethyl-pyrimidine-2,4-diamine and N2-[6-fluoro-5-[rel-(2R)-2-methyl-2,3,4,7-tetrahydro-1H-azepin-5-yl]-2,3-dihydro-1,4-benzodioxin-7-yl]-N4,6-dimethyl-pyrimidine-2,4-diamine

To a solution of tert-butyl 5-[6-fluoro-7-[[4-methyl-6-(methylamino)pyrimidin-2-yl]amino]-2,3-dihydro-1,4-benzodioxin-5-yl]-2-methyl-2,3,4,7-tetrahydroazepine-1-carboxylate (90 mg, 180.15 μmol) in DCM (2 mL) was added ZnBr₂ (202.85 mg, 900.76 μmol), then the reaction mixture was stirred at 30° C. for 12 hrs. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give crude product. The crude product was purified by SFC (basic condition, column: DAICEL CHIRALCEL OX (250 mm*30 mm*10 um); mobile phase: [0.1% NH₃H₂O MeOH; B %: 55%-55%, 15 min) to give N2-[6-fluoro-5-[rel-(2S)-2-methyl-2,3,4,7-tetrahydro-1H-azepin-5-yl]-2,3-dihydro-1,4-benzodioxin-7-yl]-N4,6-dimethyl-pyrimidine-2,4-diamine (11.3 mg, 28.29 μmol, 15.70% yield) (purity: 100%, ee: 100%) as a light-yellow solid and N2-[6-fluoro-5-[rel-(2R)-2-methyl-2,3,4,7-tetrahydro-1H-azepin-5-yl]-2,3-dihydro-1,4-benzodioxin-7-yl]-N4,6-dimethyl-pyrimidine-2,4-diamine (11.3 mg, 28.29 μmol, 15.70% yield) (purity: 100%, ee: 99.02%) as a light-yellow solid.

¹H NMR (400 MHz, CD₃OD, 298 K) δ: 7.83 (br d, J=7.3 Hz, 1H), 5.88 (br t, J=4.9 Hz, 1H), 5.82 (s, 1H), 5.38-5.37 (m, 1H), 4.21 (s, 4H), 3.55-3.41 (m, 2H), 3.06 (br d, J=2.0 Hz, 1H), 2.90 (s, 3H), 2.67-2.54 (m, 1H), 2.42 (br dd, J=7.6, 15.4 Hz, 1H), 2.17 (s, 3H), 1.96-1.85 (m, 1H), 1.69-1.55 (m, 1H), 1.18 (br d, J=6.4 Hz, 3H)

¹H NMR (400 MHz, CD₃OD, 298 K) δ: 7.82 (br dd, J=1.8, 3.4 Hz, 1H), 5.87 (br d, J=5.0 Hz, 1H), 5.82 (s, 1H), 4.20 (s, 4H), 3.57-3.44 (m, 2H), 3.06 (br d, J=1.6 Hz, 1H), 2.89 (s, 3H), 2.67-2.54 (m, 1H), 2.48-2.38 (m, 1H), 2.17 (s, 3H), 1.95-1.85 (m, 1H), 1.69-1.56 (m, 1H), 1.18 (br d, J=5.7 Hz, 3H)

### EXAMPLE 62. Synthesis of Compound 166 and Compound 165

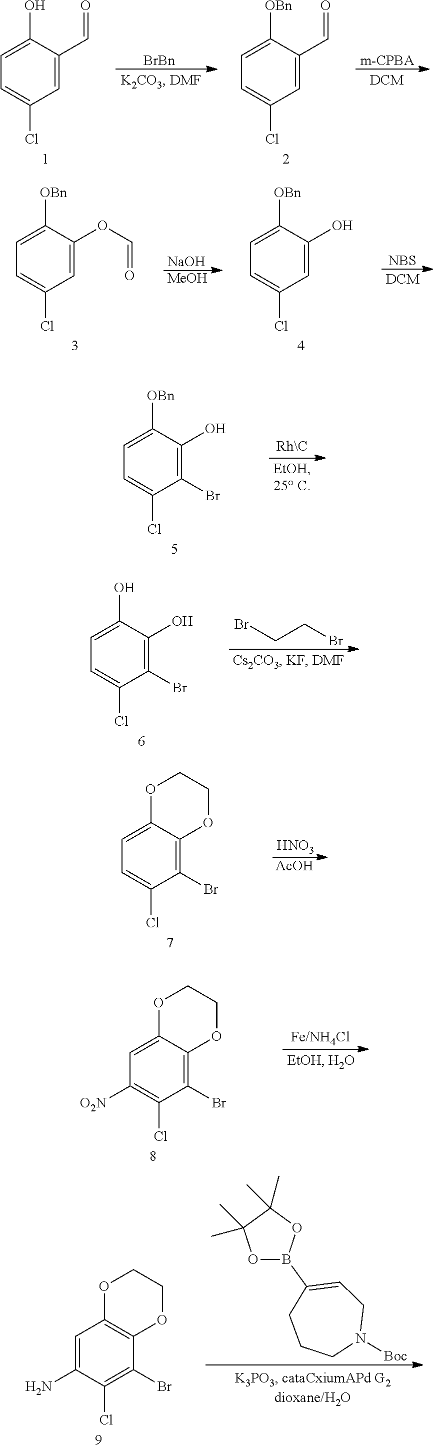

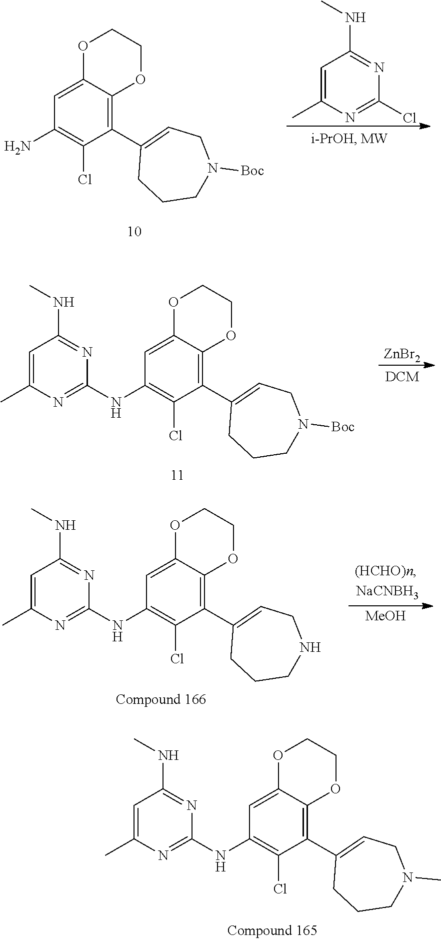

### Step 1. 2-(benzyloxy)-5-chlorobenzaldehyde

To a solution of 5-chloro-2-hydroxy-benzaldehyde (30 g, 191.61 mmol) bromoethylbenzene (49.16 g, 287.42 mmol, 34.19 mL) in DMF (300 mL) was added K₂CO₃ (40.67 g, 383.22 mmol), then the mixture was stirred at 60° C. for 12 hrs. The reaction mixture was poured into water (200 mL), extracted with EtOAc (200 mL*3). The organic layer was dried over Na₂SO₄, concentrated to give the 2-benzyloxy-5-chloro-benzaldehyde (46 g, crude) as a white solid.

¹H NMR (400 MHz, CHLOROFORM-d) δ:10.42-10.33 (m, 1H), 7.73-7.65 (m, 1H), 7.39-7.36 (m, 1H), 7.36-7.34 (m, 1H), 7.34-7.33 (m, 1H), 7.33-7.31 (m, 2H), 7.31-7.24 (m, 1H), 6.94-6.84 (m, 1H), 5.25-4.88 (m, 2H).

### Step 2. 2-(benzyloxy)-5-chlorophenyl formate

To a solution of 2-benzyloxy-5-chloro-benzaldehyde (46 g, 186.47 mmol) in DCM (500 mL) was added 3-chlorobenzenecarboperoxoic acid (53.00 g, 261.06 mmol, 85% purity) by portion-wise at 0° C. And then the reaction mixture was stirred at 40° C. for 4 h. The reaction mixture was quenched by Na₂SO₃: NaHCO₃ (v/v=300/300 mL), extracted with DCM (150 mL*3). The organic layer was dried over Na₂SO₄, concentrated to give the (2-benzyloxy-5-chloro-phenyl) formate (51 g, crude) as a yellow oil.

### Step 3. tert-butyl N-allyl-N-[2-[tert-butyl(dimethyl)silyl]oxypent-4-ynyl]carbamate

To a solution of (2-benzyloxy-5-chloro-phenyl) formate (25.5 g, 97.07 mmol) in MeOH (300 mL) was added dropwise NaOH (51 mL) (2.5M in H₂O). The mixture was stirred at 20° C. for 5 h. TLC (petroleum ether:ethyl acetate=3:1) indicated the starting material remained, and many spots were detected. The reaction mixture was concentrated to remover MeOH, the water layer was acidified by HCl (1M) till pH=3. The water layer was extracted with EtOAc (150 mL*3). The organic layer was dried over Na₂SO₄, concentrated to give the crude product. The residue was purified by column chromatography on silica gel. 2-benzyloxy-5-chloro-phenol (18 g, crude) was obtained as colorless oil.

¹H NMR (400 MHz, CHLOROFORM-d) δ: 7.50-7.37 (m, 5H), 7.02-6.96 (m, 1H), 6.89-6.75 (m, 2H), 5.19-5.01 (m, 2H).

### Step 4. 6-(benzyloxy)-2-bromo-3-chlorophenol

To a solution of 2-benzyloxy-5-chloro-phenol (5.03 g, 21.4 mmol) w as added N-isopropylpropan-2-amine (5.74 mg, 56.8 mmol, 8 mL) at room temperature, and the mixture was stirred for a while, During stirring colorless crystals were precipitated. Then, the mixture was dissolved in DCM (43 mL). To the mixture was added NBS (3.05 g, 17.2 mmol,) portion-wise over 30 min at 0° C. and the mixture was stirred for 15 min.

The reaction was quenched with saturated aq. Na₂S₂O₃ (20 mL), and then extracted with DCM (30 mL*5). The combined organic layers were dried over Na₂SO₄, filtered and concentrated under reduced pressure. The resulting residue was purified by silica gel column chromatography to give 6-benzyloxy-2-bromo-3-chloro-phenol (3.65 g, 11.64 mmol, 54.63% yield) as a yellow solid.

### Step 5. 3-bromo-4-chlorobenzene-1,2-diol

6-benzyloxy-2-bromo-3-chloro-phenol (3.65 g, 11.64 mmol) was dissolved in EtOH (200 mL), and then to the mixture was added Rh/C (1.5 g), the mixture was stirred at 25° C. for 12 h under H₂ (15 psi). The reaction was filtered, and the filtrate was concentrated in vacuum. The resulting residue was purified by silica gel column chromatography to give 3-bromo-4-chloro-benzene-1,2-diol (2.65 g, crude) as a yellow solid.

¹H NMR (400 MHz, CHLOROFORM-d) δ: 6.89 (d, J=8.8 Hz, 1H), 6.78-6.66 (m, 1H), 5.07-4.57 (m, 3H).

### Step 6. 5-bromo-6-chloro-2,3-dihydrobenzo[b][1,4]dioxine

To a solution of 3-bromo-4-chloro-benzene-1,2-diol (2.65 g, 11.85 mmol) in DMF (125 mL) was added C_{S2}CO₃ (11.58 g, 35.56 mmol), KF (688.59 mg, 11.85 mmol), 1,2-dibromoethane (4.45 g, 23.70 mmol, 2.04 mL) at 25° C., then the mixture was stirred at 100° C. for 12 hrs. The reaction mixture was poured into water (50 mL), extracted with EtOAc (50 mL*3). The combined organic layers were washed with 30 mL of brine, dried over anhydrous Na₂SO₄, filtered and concentrated under reduced pressure to give a residue. The crude product was purified by column chromatography on silica gel to give 5-bromo-6-chloro-2,3-dihydro-1,4-benzodioxine (1.88 g, 7.54 mmol, 63.58% yield) as a white solid.

¹H NMR (400 MHz, CHLOROFORM-d) δ: 6.90 (d, J=8.8 Hz, 1H), 6.73 (d, J=8.8 Hz, 1H), 4.35-4.24 (m, 2H), 4.23-4.14 (m, 2H).

### Step 7. 5-bromo-6-chloro-7-nitro-2,3-dihydrobenzo[b]1,4]dioxine

To a solution of 5-bromo-6-chloro-2,3-dihydro-1,4-benzodioxine (1.5 g, 6.01 mmol) in AcOH (25 mL) was added HNO₃ (11.25 g, 178.54 mmol, 7.50 mL) at 0° C., then the mixture was stirred at 25° C. for 3 hrs. The reaction mixture was added to ice water (30 mL), the mixture was filtered and the filter liquid was dried in vacuum to give the crude product which was purified by silica gel column chromatography to give 5-bromo-6-chloro-7-nitro-2,3-dihydro-1,4-benzodioxine as a white solid.

### Step 8. 8-bromo-7-chloro-2,3-dihydrobenzo[b][1,4]dioxin-6-amine

To a solution of 5-bromo-6-chloro-7-nitro-2,3-dihydro-1,4-benzodioxine (1.12 g, 3.80 mmol) in H₂O (6 mL), was added Fe (1.06 g, 19.02 mmol) and NH₄Cl (2.03 g, 38.03 mmol), and then the mixture was stirred at 80° C. for 2 hrs. The reaction was filtered and concentrated under reduced pressure. And then the crude product was quenched with H₂O (30 mL), and the mixture was extracted with EtOAc (30 mL*3). The combined organic layers were dried over Na₂SO₄, was filtered and concentrated under reduced pressure. The reaction mixture was used for next step directly without workup.

### Step 9. tert-butyl 5-(7-amino-6-chloro-2,3-dihydrobenzo[b i/f],4]dioxin-5-yl)-2,3,4,7-tetrahydro-1H-azepine-1-carboxylate

To a mixture of 5-bromo-6-chloro-2,3-dihydro-1,4-benzodioxin-7-amine (40 mg, 151.23 μmol), tert-butyl 5-(4,4,5,5-tetramethyl-1,3,2-dioxaborolan-2-yl)-2,3,4,7-tetrahydroazepine-1-carboxylate (58.66 mg, 181.47 mol) in THF (2 mL) and H₂O (0.5 mL) was added K₃PO₄ (64.20 mg, 302.45 mol) and cataCxiumAPd G2 (10.11 mg, 15.12 mol) at 25° C., then the mixture was stirred at 80° C. for 4 hrs under N2. The reaction mixture was filtered and concentrated to give the crude product. The crude product was purified by column chromatograph on silia gel to give tert-butyl 5-(7-amino-6-chloro-2,3-dihydro-1,4-benzodioxin-5-yl)-2,3,4,7-tetrahydroazepine-1-carboxylate (80 mg, crude) as a yellow gum.

### Step 10. 5tert-butyl 5-(6-chloro-7-((4-methyl-6-(methylamino)pyrimidin-2-yl)amino)-2,3-dihydrobenzo[b][1,4]dioxin-5-yl)-2,3,4,7-tetrahydro-1H-azepine-1-carboxylate

To a solution of tert-butyl5-(7-amino-6-chloro-2,3-dihydro-1,4-benzodioxin-5-yl)-2,3,4,7-tetrahydroazepine-1-carboxylate (70 mg, 183.79 μmol) and 2-chloro-N,6-dimethyl-pyrimidin-4-amine (28.97 mg, 183.79 μmol) in i-PrOH (1.97 mL) was added TFA (39.40 mg, 345.53 μmol, 26.62 μL), then the mixture was stirred at 140° C. for 2 h under M. w. condition. The reaction was concentrated under reduced pressure to give tert-butyl 5-[6-chloro-7-[[4-methyl-6-(methylamino)pyrimidin-2-yl]amino]-2,3-dihydro-1,4-benzodioxin-5-yl]-2,3,4,7-tetrahydroazepine-1-carboxylate (130 mg, crude) as brown solid.

### Step 11. N2-(7-chloro-8-(2,5,6,7-tetrahydro-1H-azepin-4-yl]-2,3-dihydrobenzo[b][1,4]dioxin-6-yl)-N4,6-dimethyl]pyrimidine-2,4-diamine

To a solution of tert-butyl 5-[6-chloro-7-[[4-methyl-6-(methylamino)pyrimidin-2-yl]amino]-2,3-dihydro-1,4-benzodioxin-5-yl]-2,3,4,7-tetrahydroazepine-1-carboxylate (130 mg, 258.96 μmol) in DCM (2 mL) was added ZnBr₂ (291.59 mg, 1.29 mmol), then the mixture was stirred at 30° C. for 3 hr. The reaction was filtered and A part of it was concentrated under reduced pressure to give N2-[6-chloro-5-(2,3,4,7-tetrahydro-1H-azepin-5-yl)-2,3-dihydro-1,4-benzodioxin-7-yl]-N4,6-dimethyl-pyrimidine-2,4-diamine (45 mg, crude) as yellow oil and it was named as P1 and used for next step. A part of the crude product was purified by prep-HPLC (TFA condition:column: Phenomenex Luna 80*30 mm*3 um; mobile phase:water(TFA)-ACN; B %: 1%-30%,8 min) to give product. N2-[6-chloro-5-(2,3,4,7-tetrahydro-1H-azepin-5-yl)-2,3-dihydro-1,4-benzodioxin-7-yl]-N4,6-dimethyl-pyrimidine-2,4-diamine (10.7 mg, 20.78 μmol, 8.02% yield, TFA) was obtained as a white solid.

¹H NMR (400 MHz, METHANOL-d₄) δ: 7.38-7.27 (m, 1H), 6.05-5.95 (m, 1H), 5.85-5.69 (m, 1H), 4.35-4.20 (m, 4H), 3.99-3.82 (m, 2H), 3.58-3.40 (m, 2H), 2.98-2.85 (m, 3H), 2.73-2.55 (m, 2H), 2.41-2.24 (m, 3H), 2.21-2.05 (m, 2H)

### Step 12. N2-[6-chloro-5-(1-methyl-2, 3, 4, 7-tetrahydroazepin-5-yl]-2,3-dihydro-1,4-benzodioxin-7-yl]-N4,6-dimethyl-pyrimidine-2,4-diamine

To a solution of N2-[6-chloro-5-(2,3,4,7-tetrahydro-1H-azepin-5-yl)-2,3-dihydro-1,4-benzodioxin-7-yl]-N4,6-dimethyl-pyrimidine-2,4-diamine (45 mg, 111.97 μmol) in MeOH (4 mL) was added formaldehyde (3.36 mg, 111.97 μmol, 3.10 μL), then the mixture was added sodium;cyanoboranuide (21.11 mg, 335.91 μmol) and stirred at 30° C. for 12 hrs. The reaction was filtered and concentrated under reduced pressure to give a residue. The crude product was purified by prep-HPLC (TFA condition:column: Phenomenex Luna 80*30 mm*3 um; mobile phase:water(TFA)-ACN; B %: 1%-30%, 8 min). N2-[6-chloro-5-(1-methyl-2,3,4,7-tetrahydroazepin-5-yl)-2,3-dihydro-1,4-benzodioxin-7-yl]-N4,6-dimethyl-pyrimidine-2,4-diamine (11.2 mg, 21.17 μmol, 18.91% yield, TFA) was obtained as a pale yellow solid.

¹H NMR (400 MHz, METHANOL-d₄) δ: 7.41-7.20 (m, 1H), 6.03-5.92 (m, 1H), 5.81-5.64 (m, 1H), 4.79-4.78 (m, 1H), 4.34-4.24 (m, 4H), 4.16-4.07 (m, 1H), 4.05-3.96 (m, 1H), 3.79-3.65 (m, 1H), 3.55-3.42 (m, 1H), 3.03-2.95 (m, 3H), 2.94-2.89 (m, 3H), 2.78-2.51 (m, 2H), 2.40-2.26 (m, 3H), 2.22-1.99 (m, 2H).

### EXAMPLE 63. Synthesis of Compound 167 and Compound 168

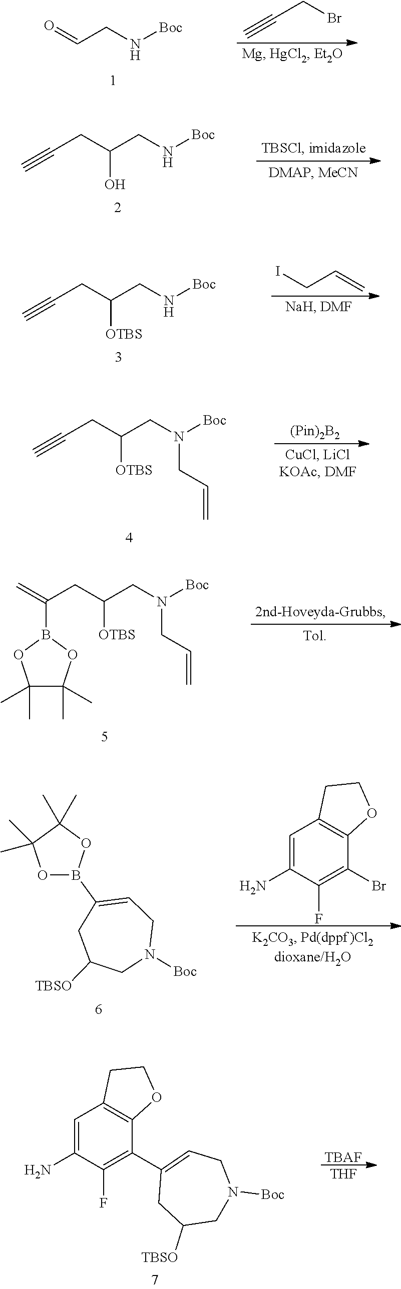

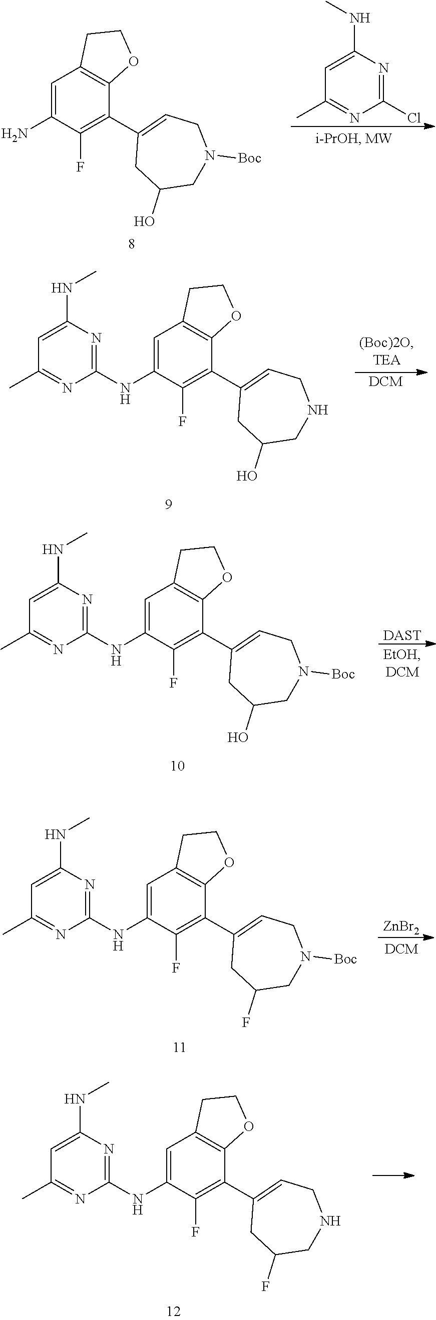

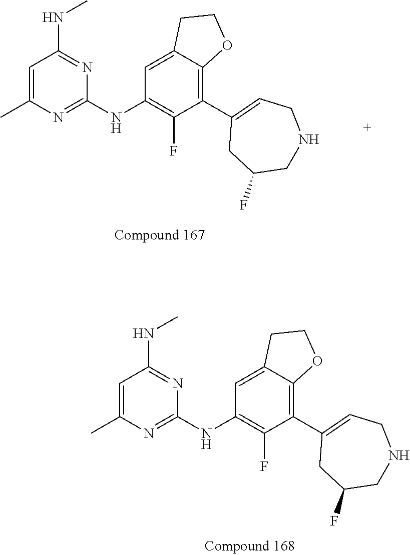 [SMILES: CNc1cc(C)nc(Nc2cc3c(c(C4=CCNC[C@H](F)C4)c2F)OCC3)n1 ; CNc1cc(C)nc(Nc2cc3c(c(C4=CCNC[C@@H](F)C4)c2F)OCC3)n1]

### Step 1. tert-butyl N-(2-hydroxypent-4-ynyl)carbamate

Mg (3.7 g, 152.23 mmol) were flame dried undervacuum, suspended in THF (55 mL), and treated with HgCl₂ (150 mg, 552.49 μmol). The mixture was stirred at 25° C. for 30 min then cooled to 4° C., and 3-bromoprop-1-yne (1.17 g, 7.85 mmol) was added. The mixture was stirred for 15 min at 25° C. and a rise in temperature was observed. The solution was maintained at 4° C. and the remainder of the 3-bromoprop-1-yne (13.45 g, 113.08 mmol) was added dropwise. The mixture was stirred at 0° C. for an additional 30 min, and then the mixture was transferred via cannula to a flask cooled to −42° C. tert-butyl N-(2-oxoethyl)carbamate (5 g, 31.41 mmol) in THF (10 mL) was added dropwise at −40° C. The reaction mixture was warmed to 25° C. for 12 h. The mixture was poured into a cold saturated NH₄Cl solution at 0° C., producing vigorous bubbling. The aqueous layer was extracted with 3*30 mL of EtOAc. The organic layers were combined and dried over Na₂SO₄, filtered, and concentrated in vacuum.

The residue was purified by flash chromatography on silica gel to give tert-butyl N-(2-hydroxypent-4-ynyl)carbamate (5 g, 25.09 mmol, 79.89% yield) as a colourless oil.

### Step 2. tert-butyl N-[2-[tert-butyl(dimethyl)silyl]oxypent-4-ynyl]carbamate

The tert-butyl N-(2-hydroxypent-4-ynyl)carbamate (1.5 g, 7.53 mmol) was dissolved in MeCN (20 mL), tert-butyl-chloro-dimethyl-silane (1.70 g, 11.29 mmol, 2.10 mL), Imidazole (1.03 g, 15.06 mmol), and N,N-dimethyl]pyridin-4-amine (1.38 g, 11.29 mmol) were then added at 25° C. after 12 h. The mixture was concentrated at 45° C., and dissolved in NaHCO₃ (50 mL), and then extracted with EtOAc (20 mL*2), the organic phase was concentrated in vacuum to give a residue which was purified by column chromatography on silica gel to give tert-butyl N-[2-[tert-butyl(dimethyl)silyl]oxypent-4-ynyl]carbamate (2.3 g, 7.34 mmol, 97.45% yield) as colourless oil.

### Step 3. ethyl 4-(tert-butoxycarbonylamino)pentanoate

tert-butyl N-[2-[tert-butyl(dimethyl)silyl]oxypent-4-ynyl]carbamate (31 g, 98.88 mmol) was dissolved in DMF (310 mL), and then to the mixture was added NaH (4.75 g, 118.66 mmol, 60% purity) at 0° C., and then the mixture was stirred at 0° C. for 30 min, 3-iodoprop-1-ene (19.93 g, 118.66 mmol, 10.85 mL) was added to the above mixture at 0° C. After addition, the mixture was stirred at 25° C. for 12 h. The reaction was quenched by sat. aq. NH₄Cl (300 mL), extracted with EtOAc (150 mL*2), and then the mixture was concentrated in vacuum to give a residue which was purified by prep-TLC to give tert-butyl N-allyl-N-[2-[tert-butyl(dimethyl)silyl]oxypent-4-ynyl]carbamate (31 g, crude) as a colourless oil.

¹H NMR (400 MHz, CHLOROFORM-d) δ: 0.02-0.13 (m, 6H) 0.85-0.94 (m, 9H) 1.39-1.52 (m, 9H) 1.95-2.05 (m, 1H) 2.29-2.43 (m, 2H) 2.92-3.06 (m, 1H) 3.43-3.55 (m, 1H)3.63-3.78 (m, 1H)3.94-4.19 (m, 2H)4.98-5.19 (m, 2H)5.65-5.85 (m, 1H)

### Step 4. tert-butyl N-allyl-N-[2-[tert-butyl(dimethyl)silyl]oxy-4-(4,4,5,5-tetramethyl-1,3,2-dioxaborolan-2-yl)pent-4-enyl]carbamate

A mixture of LiCl (8.20 mg, 193.46 μmol, 3.97 μL) and CuCl (19.15 mg, 193.46 mol) in DMF (2 mL) was stirred at 25° C. for 1 hr under N2, then the solution was added tert-butyl N-allyl-N-[2-[tert-butyl(dimethyl)silyl]oxypent-4-ynyl]carbamate (57 mg, 161.21 μmol), 4,4,5,5-tetramethyl-2-(4,4,5,5-tetramethyl-1,3,2-dioxaborolan-2-yl)-1,3,2-dioxaborolane (49.13 mg, 193.46 μmol) and KOAc (54.57 mg, 193.46 μmol) and then stirred at 25° C. for 12 hrs under N₂. The reaction was quenched by NH₄Cl (20 mL), extracted with EtOAc (5 mL*3). The organic layer was dried over Na₂SO₄, concentrated to give the crude product. The crude product was purified by prep-TLC to give tert-butyl N-allyl-N-[2-[tert-butyl(dimethyl)silyl]oxy-4-(4,4,5,5-tetramethyl-1,3,2-dioxaborolan-2-yl)pent-4-enyl]carbamate (20 mg, 41.53 mol, 25.76% yield) as clear oil.

### Step 5. tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate

tert-butyl N-allyl-N-[2-[tert-butyl(dimethyl)silyl]oxy-4-(4,4,5,5-tetramethyl-1,3,2-dioxaborolan-2-yl)pent-4-enyl]carbamate (1 g, 2.08 mmol) was dissolved in DCM (150 mL), and then to the mixture was added Hoveyda-Grubbs Catalyst 2nd Generation (65.06 mg, 103.83 μmol), and then the mixture was stirred at 25° C. for 24 hrs. The mixture was concentrated in vacuum to give a residue. The residue was purified by column chromatography on silica gel.

tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (580 mg, crude) was obtained as colourless oil.

### Step 6. tert-butyl 5-(5-amino-6-fluoro-2,3-dihydrobenzofuran-7-yl)-3-[tertbutyl(dimethyl)silyl]oxy-2,3,4,7-tetrahydroazepine-1-carboxylate

To a solution of tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (1 g, 2.21 mmol) and 7-bromo-6-fluoro-2,3-dihydrobenzofuran-5-amine (511.69 mg, 2.21 mmol) in dioxane (20 mL) and H₂O (2 mL) was added cyclopentyl(diphenyl)phosphane;dichloropalladium;iron (161.35 mg, 220.51 mol) and tripotassium;carbonate (609.51 mg, 4.41 mmol), and then the reaction mixture was stirred at 100° C. for 12 hrs under N2. The reaction was filtered and concentrated under reduced pressure to give a residue. The residue was purified by column chromatography on silica gel to give tert-butyl 5-(5-amino-6-fluoro-2,3-dihydrobenzofuran-7-yl)-3-[tert-butyl(dimethyl)silyl]oxy-2,3,4,7-tetrahydroazepine-1-carboxylate (820 mg, crude) as a light yellow oil.

### Step 7. tert-butyl 5-(5-amino-6-fluoro-2,3-dihydrobenzofuran-7-yl)-3-hydroxy-2,3,4,7-tetrahydroazepine-1-carboxylate

To a solution of tert-butyl 5-(5-amino-6-fluoro-2,3-dihydrobenzofuran-7-yl)-3-[tert-butyl(dimethyl)silyl]oxy-2,3,4,7-tetrahydroazepine-1-carboxylate (340 mg, 710.30 μmol) in THF (4 mL) was added tetrabutylammonium;fluoride (2.62 g, 10.03 mmol, 2.90 mL) then the reaction mixture was stirred at 25° C. for 12 hrs. The reaction mixture was added to water (4 mL), extracted with EtOAc (4 mL*3). The organic layer was dried over Na₂SO₄, concentrated to give tert-butyl 5-(5-amino-6-fluoro-2,3-dihydrobenzofuran-7-yl)-3-hydroxy-2,3,4,7-tetrahydroazepine-1-carboxylate (250 mg, crude) as a light yellow oil.

### Step 8. 5-[6-fluoro-5-[[4-methyl-6-(methylamino)pyrimidin-2-yl]amino]-2,3-dihydrobenzofuran-7-yl]-1-2,3,4,7-tetrahydro-1H-azepin-3-ol

To a solution of tert-butyl 5-(5-amino-6-fluoro-2,3-dihydrobenzofuran-7-yl)-3-hydroxy-2,3,4,7-tetrahydroazepine-1-carboxylate (500 mg, 1.37 mmol) in i-PrOH (10.00 mL) was added 2-chloro-N,6-dimethyl-pyrimidin-4-amine (216.24 mg, 1.37 mmol) and TFA (15.64 mg, 137.21 μmol, 10.57 μL). Then the reaction mixture was stirred at 150° C. for 12 h in a 100 mL of autoclave. The reaction mixture was concentrated to give 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (1.65 g, crude) as a brown oil.

### Step 9. tert-butyl 5-[6-fluoro-5-[[4-methyl-6-(methylamino)pyrimidin-2-yl]amino]-2,3-dihydrobenzofuran-7-yl]-3-hydroxy-2,3,4,7-tetrahydroazepine-1-carboxylate

To a solution of 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (1.65 g, 4.28 mmol) and TEA (1.52 g, 14.98 mmol, 2.09 mL) in DCM (17 mL) was added tert-butoxycarbonyl tert-butyl carbonate (1.17 g, 5.35 mmol, 1.23 mL) at 20° C., then the mixture was stirred at 20° C. for 12 h.

The reaction mixture was added to water (20 mL), extracted with DCM (10 mL*3). The organic layer was dried over Na₂SO₄, concentrated to give the crude product. The residue was purified by column chromatography on silica gel tert-butyl 5-[6-fluoro-5-[[4-methyl-6-(methylamino)pyrimidin-2-yl]amino]-2,3-dihydrobenzofuran-7-yl]-3-hydroxy-2,3,4,7-tetrahydroazepine-1-carboxylate (450 mg, crude) as brown oil.

### Step 10. tert-butyl 3-fluoro-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate

To a solution of tert-butyl 5-[6-fluoro-5-[[4-methyl-6-(methylamino)pyrimidin-2-yl]amino]-2,3-dihydrobenzofuran-7-yl]-3-hydroxy-2,3,4,7-tetrahydroazepine-1-carboxylate (230 mg, 473.69 mol) in EtOH (0.1 mL) and DCM (1 mL) was added BAST (786.00 mg, 3.55 mmol) at −10° C. under N2, then the mixture was stirred at −10° C. for 2 h under N2. The reaction mixture was quenched by MeOH (1 mL). The reaction mixture was concentrated to give the crude product. The crude product was purified by prep-HPLC(TFA condition:column:Phenomenex Luna 80*30 mm*3 um; mobile phase:water(TFA)-ACN; B %: 20%-50%,8 min) to give tert-butyl 3-fluoro-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (60 mg, 123.07 μmol, 25.98% yield) as white solid.

### Step 11. N2-[6-fluoro-7-(3-fluoro-2,3,4,7-tetrahydro-1H-azepin-5-1-yl]-2,3-dihydrobenzofuran-5-yl]-N4,6-dimethyl-pyrimidine-2,4-diamine

To a solution of tert-butyl 3-fluoro-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (30 mg, 61.53 mol) in DCM (1 mL) was added ZnBr₂ (69.29 mg, 307.67 mol), then the mixture was stirred at 30° C. for 12 h. The reaction mixture was concentrated to give the crude product.

The crude product was purified by prep-HPLC(TFA condition:column:Phenomenex Luna 80*30 mm*3 um; mobile phase:water(TFA)-ACN; B %: 1%-30%,8 min) to N2-[6-fluoro-7-(3-fluoro-2,3,4,7-tetrahydro-1H-azepin-5-yl)-2,3-dihydrobenzofuran-5-yl]-N4,6-dimethyl-pyrimidine-2,4-diamine (20 mg, 51.62 μmol, 83.89% yield) as white solid.

### Step 12. N2-[6-fluoro-7-[rel-(3R)-3-fluoro-2,3,4,7-tetrahydro-1H-azepin-5-yl]-2,3-dihydrobenzofuran-5-yl]-N4,6-dimethyl-pyrimidine-2,4-diamine and N2-[6-fluoro-7-[rel-(3S)-3-fluoro-2,3,4,7-tetrahydro-1H-azepin-5-yl]-2,3-dihydrobenzofuran-5-yl]-N4,6-dimethyl-pyrimidine-2,4-diamine

The mixture was separated by Chiral SFC (column: Lux Cellulose-2, 50×4.6 mm I.D.; mobile phase: A: CO2 B:MeOH(0.1% IPAm, v/v; SFC, Acq Method: C2_MeOH_IPAm_50_4_35) to give N2-[6-fluoro-7-[rel-(3R)-3-fluoro-2,3,4,7-tetrahydro-1H-azepin-5-yl]-2,3-dihydrobenzofuran-5-yl]-N4,6-dimethyl-pyrimidine-2,4-diamine (1.1 mg, 2.84 μmol, 4.94% yield)(de %=100%) was obtained as a white solid and N2-[6-fluoro-7-[rel-(3S)-3-fluoro-2,3,4,7-tetrahydro-1H-azepin-5-yl]-2,3-dihydrobenzofuran-5-yl]-N4,6-dimethyl-pyrimidine-2,4-diamine (1.1 mg, 2.84 μmol, 4.94% yield)(de %=100%) was obtained as a pale yellow solid.

¹H NMR (400 MHz, METHANOL-d₄) δ: 7.88-7.71 (m, 1H), 6.17-6.00 (m, 1H), 5.88-5.71 (m, 1H), 4.82-4.66 (m, 1H), 4.62-4.53 (m, 2H), 3.39 (br s, 3H), 3.24-3.00 (m, 4H), 2.85 (d, J=3.3 Hz, 4H), 2.13 (s, 3H)

¹H NMR (400 MHz, METHANOL-d₄) δ: 7.90-7.78 (m, 1H), 6.13-6.01 (m, 1H), 5.84-5.75 (m, 1H), 4.82-4.63 (m, 1H), 4.62-4.52 (m, 2H), 3.49-3.32 (m, 3H), 3.22-3.15 (m, 2H), 3.10-2.99 (m, 2H), 2.88-2.72 (m, 4H), 2.22-2.10 (m, 3H)

### EXAMPLE 64. Synthesis of Compound 351

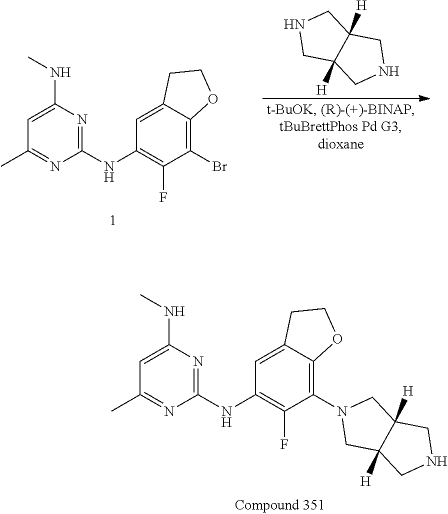 [SMILES: CNc1cc(C)nc(Nc2cc3c(c(Br)c2F)OCC3)n1 ; C1NC[C@H]2CNC[C@@H]12 ; CNc1cc(C)nc(Nc2cc3c(c(N4C[C@H]5CNC[C@H]5C4)c2F)OCC3)n1]

### Step 1. N2-[6-fluoro-7-[rac-(3aS,6aR)-2,3,3a, 4,6,6a-hexahydro-1H-pyrrolo[3,4-c]pyrrol-5-yl]-2,3-dihydrobenzofuran-5-yl]-N4,6-dimethyl-pyrimidine-2,4-diamine

To a mixture of N2-(7-bromo-6-fluoro-2,3-dihydrobenzofuran-5-yl)-N4,6-dimethyl-pyrimidine-2,4-diamine (20 mg, 56.63 mol) in t-amylOH (1 mL) was added 1,2,3,3a,4,5,6,6a-octahydropyrrolo[3,4-c]pyrrole (6.35 mg, 56.63 mol), tBuBrettPhos Pd G3 (4.84 mg, 5.66 mol), C_{S2}CO₃ (36.90 mg, 113.25 mol), then the mixture was stirred at 90° C. for 12 hrs under N₂ atmosphere. The reaction mixture was filtered and concentrated under reduced pressure to give a crude product. The residue was purified by prep-HPLC (TFA condition; column: Phenomenex luna C18 80*30 mm*3 um; mobile phase: [water(0.1% TFA)-ACN]; B %: 5%-35%,5 min). N2-[6-fluoro-7-[rac-(3aS,6aR)-2,3,3a,4,6,6a-hexahydro-1H-pyrrolo[3,4-c]pyrrol-5-yl]-2,3-dihydrobenzofuran-5-yl]-N4,6-dimethyl-pyrimidine-2,4-diamine (18 mg, 46.82 μmol, 82.68% yield) was obtained as a pale yellow solid.

¹H NMR (400 MHz, METHANOL-d₄) δ: 2.29 (s, 3H) 2.90-2.95 (m, 3H) 3.05-3.14 (m, 4H) 3.18-3.23 (m, 2H) 3.31-3.35 (m, 2H) 3.38-3.48 (m, 4H) 3.61-3.66 (m, 2H) 4.64 (t, J=8.69 Hz, 2H) 4.88 (s, 11H) 5.99 (s, 1H) 7.06-7.16 (m, 1H).

### EXAMPLE 65. Synthesis of Compound 352 and Compound 382

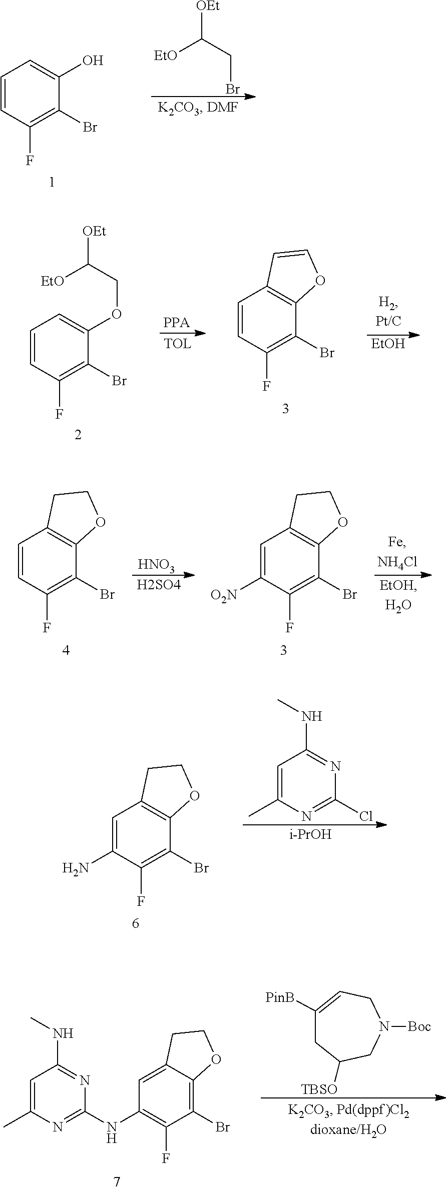

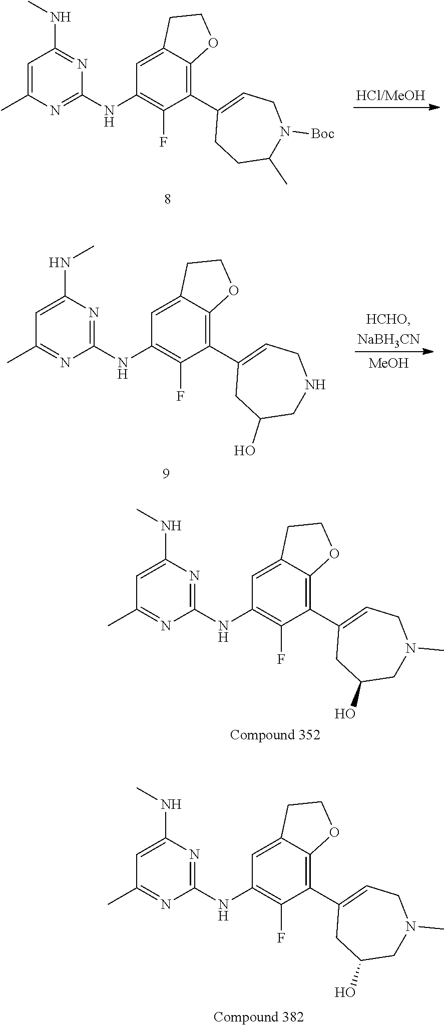

### Step 1.2-bromo-1-(2,2-diethoxyethoxy)-3-fluoro-benzene

2-bromo-3-fluoro-phenol (10 g, 52.36 mmol) was dissolved in DMF (500 mL), and then to the mixture was added K₂CO₃ (10.85 g, 78.54 mmol) and 2-bromo-1,1-diethoxy-ethane (41.27 g, 209.43 mmol), and then the mixture was stirred at 110° C. for 12 h. The mixture was concentrated in vacuo. The residue was purified by flash chromatography 2-bromo-1-(2,2-diethoxyethoxy)-3-fluoro-benzene (80 g, crude) was obtained as a yellow oil.

### Step 2. 7-bromo-6-fluoro-benzofuran

2-bromo-1-(2,2-diethoxyethoxy)-3-fluoro-benzene (16 g, 52.09 mmol) was dissolved in toluene (800 mL), and then to the mixture was added PPA (40 g, 52.09 mmol), and then the mixture was stirred at 110° C. for 2 h. The mixture was concentrated in vacuum. The residue was purified by flash chromatography 7-bromo-6-fluoro-benzofuran (30 g, crude) was obtained as a yellow oil.

### Step 3. 7-bromo-6-fluoro-2,3-dihydrobenzofuran

7-bromo-6-fluoro-benzofuran (8 g, 37.21 mmol) was dissolved in EtOH (400 mL) and then to the mixture was added Pt\C (10 g, 5% purity), and then the mixture was stirred at 25° C. for 3 hrs under H₂ (37.21 mmol) (15 psi). The mixture was filtered, and the filtrated was concentrated to give 7-bromo-6-fluoro-2,3-dihydrobenzofuran (20 g, crude) as a yellow solid.

### Step 4. 7-bromo-6-fluoro-5-nitro-2,3-dihydrobenzofuran

7-bromo-6-fluoro-2,3-dihydrobenzofuran (20 g, 92.15 mmol) was dissolved in TFA (200 mL), and then to the mixture was added HNO₃ (8.71 g, 138.23 mmol, 1 mL) at 0° C.-10° C., and the mixture was stirred at 25° C. for 1 hr. The suspension was added to ice (200 mL), and then the suspension was filtered, and the filter cake was washed with H₂O (100 mL*3), and then concentrated in vacuo. 7-bromo-6-fluoro-5-nitro-2,3-dihydrobenzofuran (24 g, 91.59 mmol, 99.39% yield) was obtained as a yellow solid.

### Step 5. 7-bromo-6-fluoro-2,3-dihydrobenzofuran-5-amine

7-bromo-6-fluoro-5-nitro-2,3-dihydrobenzofuran (4 g, 15.27 mmol) was dissolved in EtOH (500 mL) and then to the mixture was added Rh/C (6.00 g, 2.54 mmol, 5% purity), and then the mixture was stirred at 25° C. for 2 hrs under H₂ (15 psi). The mixture was filtered, and the filtrated was concentrated in vacuum to give 7-bromo-6-fluoro-2,3-dihydrobenzofuran-5-amine (5.6 g, crude) as a yellow solid.

### Step 6. N2-(7-bromo-6-fluoro-2,3-dihydrobenzofuran-5-yl)-N4,6-dimethyl-pyrimidine-2,4-diamine

To the mixture of 7-bromo-6-fluoro-2,3-dihydrobenzofuran-5-amine (250 mg, 1.08 mmol),2-chloro-N,6-dimethyl-pyrimidin-4-amine (203.75 mg, 1.29 mmol) in i-PrOH (10 mL) and TFA (0.1 mL), then stirred at 130° C. for 2 hrs. The reaction was concentrated under reduced pressure to give N2-(7-bromo-6-fluoro-2,3-dihydrobenzofuran-5-yl)-N4,6-dimethyl-pyrimidine-2,4-diamine (2.8 g, crude) as brown solid.

### Step 7. tert-butyl 3-[tert-butyl(dimethyl)silyl]oxy-5-[6-fluoro-5-[[4-methyl-6-(methylamino)pyrimidin-2-yl]amino]-2,3-dihydrobenzo furan-7-yl]-2,3,4,7-tetrahydroazepine-1-carboxylate

To a mixture of N2-(7-bromo-6-fluoro-2,3-dihydrobenzofuran-5-yl)-N4,6-dimethyl-pyri midine-2,4-diamine (200 mg, 566.27 mol) and tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (256.80 mg, 566.27 mol) in dioxane (10 mL) and H₂O (1 mL) was added K₂CO₃ (156.52 mg, 1.13 mmol) and cyclopentyl(diphenyl)phosphane dichloromethane;dichloropalladium;iron (46.24 mg, 56.63 mol) under N₂ atmosphere. It was stirred at 100° C. for 12 hrs under N₂ atmosphere. The reaction was filtered and concentrated under reduced pressure to give tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (crude) was obtained as a brown oil.

### Step 8. 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (100 mg, crude) 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol

A solution of tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (156 mg, 260.08 mol) in HCl/MeOH (5.0 mL). The reaction mixture was drying to give 5-[6-fluoro-5-[[4-methyl-6-(methylamino) pyrimidin-2-yl]amino]-2,3-dihydrobenzofuran-7-yl]-2,3,4,7-tetrahydro-1H-azepin-3-ol(100 mg, crude) 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (100 mg, crude) as a brown gum.

### Step 9. rel-(3S)-5-[6-fluoro-5-[[4-methyl-6-(methylamino)pyrimidin-2-yl]amino]-2,3-dihydrobenzofuran-7-yl]-1-methyl-2,3,4,7-tetrahydroazepin-3-ol and rel-(3R)-5-[6-fluoro-5-[[4-methyl-6-(methylamino)pyrimidin-2-yl]amino]-2,3-dihydrobenzofuran-7-yl]-1-methyl-2,3,4,7-tetrahydroazepin-3-ol

To a mixture of 5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (100 mg, 259.45 μmol) and formaldehyde (7.79 mg, 259.45 μmol, 7.19 μL) in MeOH (4 mL) was added sodium;cyanoboranuide (48.91 mg, 778.34 μmol), then stirred at 25° C. for 12 hrs. The reaction mixture was concentrated and purified by prep-HPLC (TFA condition:column: Phenomenex Luna 80*30 mm*3 um; mobile phase:water(TFA)-ACN; B %: 1%-25%,8 min) to give the mixture of these two single isomer. The mixture was separated by SFC (column: DAICEL CHIRALPAKIC (250 mm*30 mm,10 um); mobile phase:[0.1% NH₃H₂O MEOH]; B %: 50%-55%; SFC, Acq Method: IC_MeOH_IPAm_5_50_3_35_3 min) to give rel-(3S)-5-[6-fluoro-5-[[4-methyl-6-(methylamino)pyrimidin-2-yl]amino]-2,3-dihydrobenzofuran-7-yl]-1-methyl-2,3,4,7-tetrahydroazepin-3-ol (12.6 mg, 31.54 μmol, 12.16% yield, Rt=1.449 min) as a yellow solid and rel-(3R)-5-[6-fluoro-5-[[4-methyl-6-(methylamino)pyrimidin-2-yl]amino]-2,3-dihydrobenzofuran-7-yl]-1-methyl-2,3,4,7-tetrahydroazepin-3-ol (17.5 mg, 43.81 μmol, 16.89% yield, Rt=2.113 min) as a yellow oil.

¹H NMR (400 MHz, METHANOL-d₄) δ: 2.13-2.18 (m, 3H) 2.39-2.44 (m, 3H) 2.56-2.65 (m, 2H) 2.80 (br d, J=12.38 Hz, 1H) 2.84-2.87 (m, 3H) 3.11-3.26 (m, 6H) 3.95-4.03 (m, 1H) 4.51-4.62 (m, 2H) 5.76-5.81 (m, 1H) 5.97-6.04 (m, 1H) 7.80-7.86 (m, 1H)

¹H NMR (400 MHz, METHANOL-d₄) δ: 2.20-2.29 (m, 3H) 2.86 (s, 3H) 2.87-3.00 (m, 4H) 3.22-3.27 (m, 2H) 3.46-3.55 (m, 1H) 3.78-3.93 (m, 2H) 4.10-4.17 (m, 1H) 4.65 (td, J=8.72, 4.19 Hz, 2H) 5.91-5.97 (m, 1H) 6.02-6.09 (m, 1H) 7.54 (br d, J=7.38 Hz, 1H)

### EXAMPLE 66. Synthesis of Compound 353

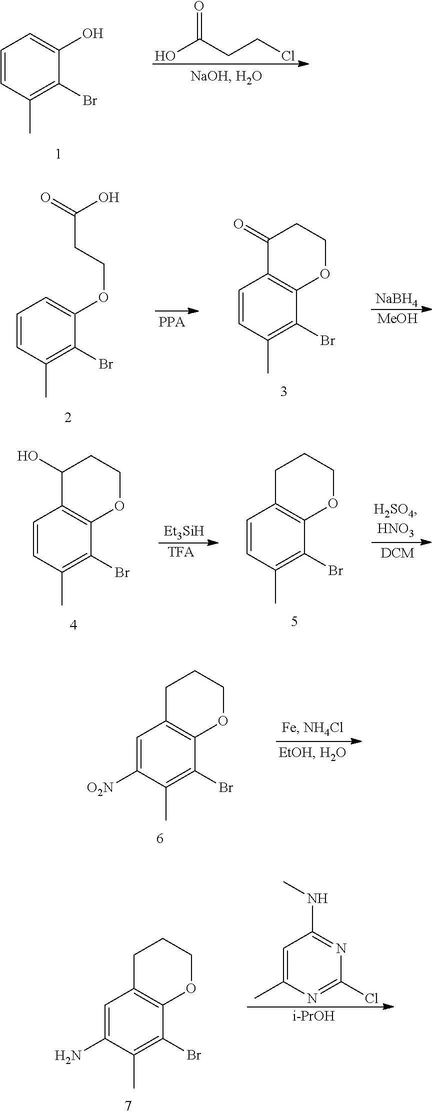

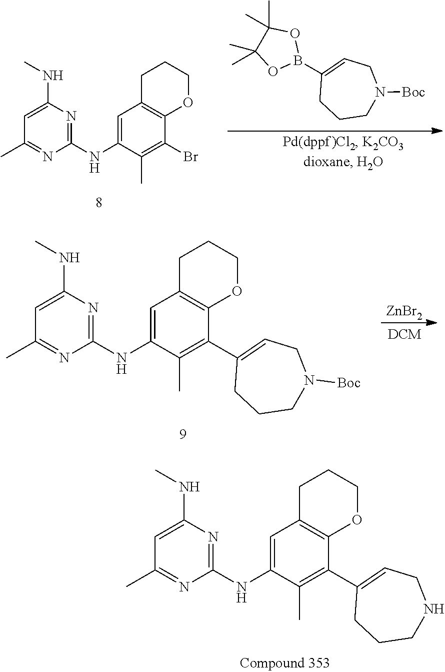

### Step 1. 3-(2-bromo-3-methyl-phenoxy)propanoic acid

A mixture of 2-bromo-3-methyl-phenol (10 g, 53.47 mmol) and 3-chloropropanoic acid (5.80 g, 53.47 mmol, 4.57 mL), then was added dropwise a solution of NaOH (5.13 g, 128.32 mmol, 2.41 mL) in H₂O (30 mL) at 20° C., then the mixture was stirred at 100° C. for 12 h. The reaction was added 3 N HCl to pH=4, then extracted with ethyl acetate (100 mL*3) and dried over anhydrous sodium sulfate, filtered and concentrated under reduced pressure to give a residue. The residue was purified by flash chromatography on silica gel to give 3-(2-bromo-3-methyl-phenoxy)propanoic acid (3.7 g, 14.28 mmol, 26.71% yield) as a yellow solid.

¹H NMR (400 MHz, METHANOL-d₄) δ: 2.38 (s, 3H) 2.80 (t, J=6.19 Hz, 2H) 4.27 (t, J=6.19 Hz, 2H) 6.88 (t, J=8.57 Hz, 2H) 7.17 (t, J=7.88 Hz, 1H)

### Step 2. 8-bromo-7-methyl-chroman-4-one

A solution of 3-(2-bromo-3-methyl-phenoxy)propanoic acid (3.5 g, 13.51 mmol) in PPA (35 mL), then the mixture was stirred at 100° C. for 2 h. The reaction was added into water (200 mL), then extracted with ethyl acetate (200*3 mL) and dried over anhydrous sodium sulfate, filtered and concentrated under reduced pressure to give 8-bromo-7-methyl-chroman-4-one (3.4 g, crude) as a brown oil.

¹H NMR (400 MHz, METHANOL-d₄) δ: 2.45 (s, 3H) 2.77-2.86 (m, 2H) 4.60-4.67 (m, 2H) 7.00 (d, J=8.00 Hz, 1H) 7.72 (d, J=8.00 Hz, 1H)

### Step 3. 8-bromo-7-methyl-chroman-4-ol

To a solution of 8-bromo-7-methyl-chroman-4-one (3.3 g, 13.69 mmol) in MeOH (35 mL) was added NaBH₄ (776.79 mg, 20.53 mmol) at 0° C., then the mixture was stirred at 20° C. for 1 h. The reaction was concentrated under reduced pressure, then added water (50 mL), then extracted with ethyl acetate (50 mL*3) and dried over anhydrous sodium sulfate, filtered and concentrated under reduced pressure to give 8-bromo-7-methyl-chroman-4-ol (3.3 g, crude) as a yellow solid.

¹H NMR (400 MHz, METHANOL-d₄) δ: 1.95-2.01 (m, 1H) 2.03-2.14 (m, 1H) 2.35 (s, 3H) 4.25-4.39 (m, 2H) 4.72 (t, J=4.25 Hz, 1H) 6.84 (d, J=7.88 Hz, 1H) 7.19 (d, J=7.75 Hz, 1H)

### Step 4. 8-bromo-7-methyl-chromane

To a solution of 8-bromo-7-methyl-chroman-4-ol (3.2 g, 13.16 mmol) in TFA (35 mL) was added triethylsilane (3.06 g, 26.33 mmol, 4.20 mL), then the mixture was stirred at 60° C. for 12 h. The reaction was concentrated under reduced pressure, then added water (50 mL), then extracted with ethyl acetate (50 mL*3). The combined organic layers were washed with brine (50 mL) and dried over anhydrous sodium sulfate, filtered and concentrated under reduced pressure to give a residue. The residue was purified by flash chromatography on silica gel to give 8-bromo-7-methyl-chromane (1.69 g, 7.44 mmol, 56.53% yield) as a colorless oil.

¹H NMR (400 MHz, METHANOL-d₄) δ: 1.91-1.98 (m, 2H) 2.25 (s, 3H) 2.72 (t, J=6.44 Hz, 2H) 4.08-4.15 (m, 2H) 6.65 (s, 1H) 7.17 (s, 1H)

### Step 5. 8-bromo-7-methyl-6-nitro-chromane

To a solution of 8-bromo-7-methyl-chromane (100 mg, 440.34 μmol) in DCM (2 mL) was added a mixture of nitric acid (29.13 mg, 462.36 μmol) and sulfuric acid (172.75 mg, 1.76 mmol) at −5° C., then the mixture was stirred at 20° C. for 1 h. The reaction was added into water (5 mL) at 0° C., then extracted with dichloromethane (10 mL*3). The combined organic layers were washed with saturated sodium bicarbonate solution (10 mL*3) and dried over anhydrous sodium sulfate, filtered and concentrated under reduced pressure to give 8-bromo-7-methyl-6-nitro-chromane (35 mg, 128.63 μmol, 29.21% yield) as colorless oil.

¹H NMR (400 MHz, METHANOL-d₄) δ: 2.00-2.06 (m, 2H) 2.57 (s, 3H) 2.87 (t, J=6.44 Hz, 2H) 4.34-4.41 (m, 2H) 7.71 (s, 1H)

### Step 6. 8-bromo-7-methyl-chroman-6-amine

To a solution of 8-bromo-7-methyl-6-nitro-chromane (35 mg, 128.63 μmol) and NH₄Cl (68.81 mg, 1.29 mmol) in EtOH (2 mL) and H₂O (0.2 mL) was added Fe (35.92 mg, 643.16 μmol), then the mixture was stirred at 80° C. for 12 h. The reaction was filtered and concentrated under reduced pressure, then added water (5 mL), then extracted with ethyl acetate (10 mL*3) and dried over anhydrous sodium sulfate, filtered and concentrated under reduced pressure to give 8-bromo-7-methyl-chroman-6-amine (30 mg, crude) as brown oil.

### Step 7. N2-(8-bromo-7-methyl-chroman-6-yl)-N4,6-dimethyl-pyrimidine-2,4-diamine

To a solution of 8-bromo-7-methyl-chroman-6-amine (30 mg, 123.91 μmol) and 2-chloro-N,6-dimethyl-pyrimidin-4-amine (19.53 mg, 123.91 μmol) in i-PrOH (1.5 mL) was added TFA (1.41 mg, 12.39 μmol), then the mixture was stirred at 130° C. for 3 h under sealed tube. LCMS showed starting material was consumed completely and mass of the desired compound. The reaction was concentrated under reduced pressure to give N2-(8-bromo-7-methyl-chroman-6-yl)-N4,6-dimethyl-pyrimidine-2,4-diamine (45 mg, crude) as a pink solid.

### Step 8. tert-butyl 5-[7-methyl-6-[[4-methyl-6-(methylamino)pyrimidin-2-yl]amino]chroman-8-yl]-2,3,4,7-tetrahydroazepine-1-carboxylate

To a solution of N2-(8-bromo-7-methyl-chroman-6-yl)-N4,6-dimethyl-pyrimidine-2,4-diamine (45 mg, 123.88 μmol), tert-butyl 5-(4,4,5,5-tetramethyl-1,3,2-dioxaborolan-2-yl)-2,3,4,7-tetrahydroazepine-1-carboxylate (44.05 mg, 136.27 μmol), K₂CO₃ (85.61 mg, 619.41 μmol) and H₂O (0.3 mL) in dioxane (3 mL) was added Pd(dppf)Cl₂ (9.06 mg, 12.39 μmol), then the mixture was stirred at 100° C. for 6 h under an atmosphere of nitrogen. The reaction was concentrated filtered and concentrated under reduced pressure to give tert-butyl 5-[7-methyl-6-[[4-methyl-6-(methylamino)pyrimidin-2-yl]amino]chroman-8-yl]-2,3,4,7-tetrahydroazepine-1-carboxylate (60 mg, crude) as black oil.

### Step 9. N4,6-dimethyl-N2-[7-methyl-8-(2,3,4,7-tetrahydro-1H-azepin-5-yl)chroman-6-yl]pyrimi-dine-2,4-diamine

To a solution of tert-butyl 5-[7-methyl-6-[[4-methyl-6-(methylamino)pyrimidin-2-yl]amino]chroman-8-yl]-2,3,4,7-tetrahydroazepine-1-carboxylate (60 mg, 125.10 μmol) in DCM (3 mL) was added ZnBr₂ (140.86 mg, 625.50 μmol), then the mixture was stirred at 35° C. for 12 h. The reaction was filtered and concentrated under reduced pressure to give a residue. The residue was purified by prep-HPLC (TFA condition, column: Phenomenex Luna 80*30 mm*3 um; mobile phase: [water(TFA)-ACN; B %: 10%-30%, 8 min) to give N4,6-dimethyl-N2-[7-methyl-8-(2,3,4,7-tetrahydro-1H-azepin-5-yl)chroman-6-yl]pyrimidine-2,4-diamine (28.8 mg, 58.48 μmol, 46.74% yield, TFA) (purity: 97.841%) as alight-yellow solid.

¹H NMR (400 MHz, METHANOL-d₄) δ:1.95-2.02 (m, 2H) 2.02-2.08 (m, 1H) 2.13 (s, 3H) 2.15-2.23 (m, 1H) 2.24 (s, 3H) 2.51-2.67 (m, 2H) 2.79 (br t, J=6.44 Hz, 2H) 2.85-2.99 (m, 3H) 3.36-3.44 (m, 1H) 3.56 (ddd, J=13.01, 6.44, 3.19 Hz, 1H) 3.81-3.97 (m, 2H) 4.15-4.25 (m, 2H) 5.62 (t, J=6.07 Hz, 1H) 5.92 (d, J=0.63 Hz, 1H) 7.02 (s, 1H)

### EXAMPLE 67. Synthesis of Compound 354-a and Compound 354

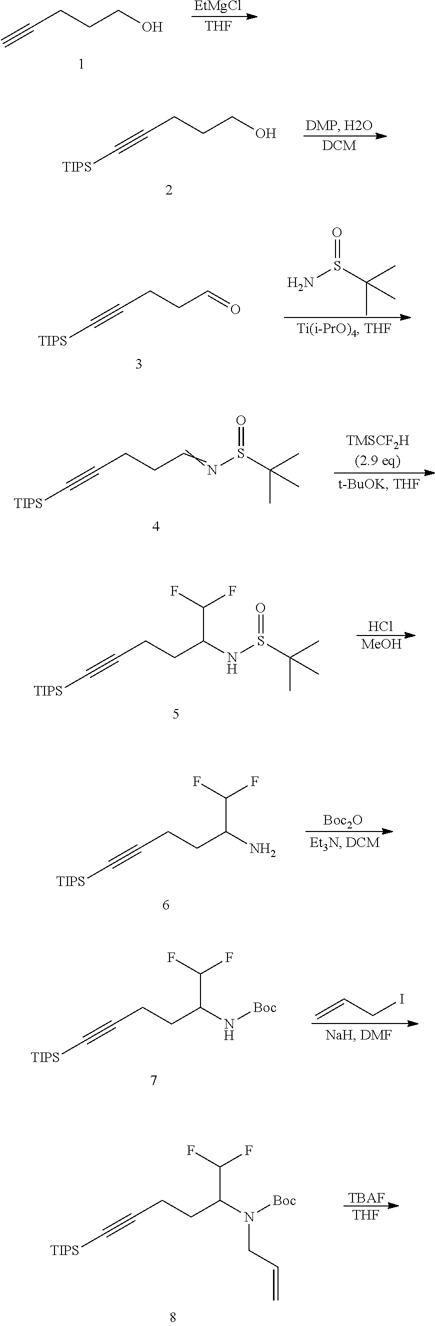

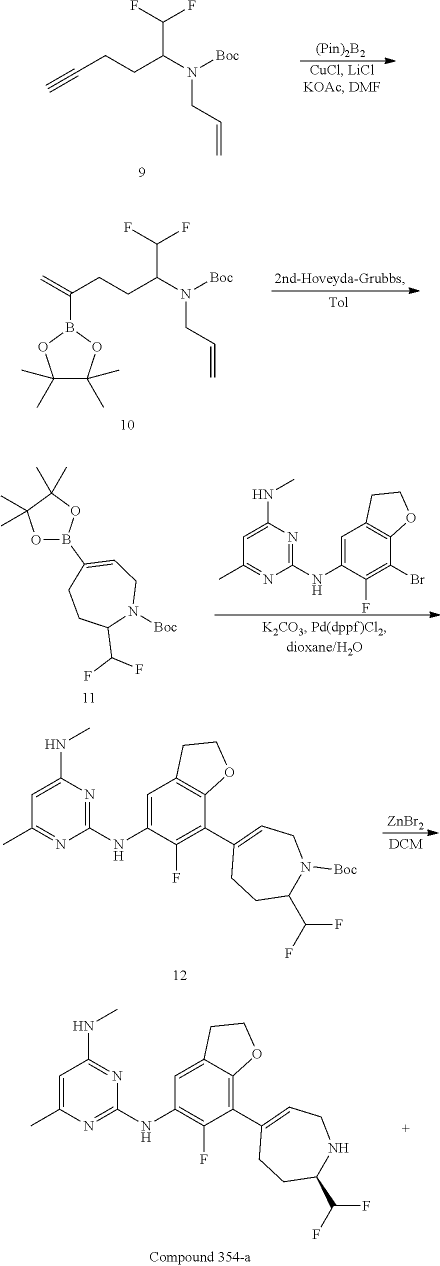

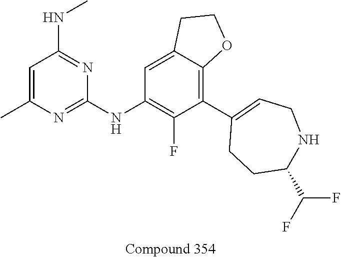 [SMILES: CNc1cc(C)nc(Nc2cc3c(c(C4=CCN[C@H](C(F)F)CC4)c2F)OCC3)n1]

### Step 1. 5-triisopropylsilylpent-4-yn-1-ol

To a solution of pent-4-yn-1-ol (50 g, 594.42 mmol) in THF (1000 mL) was added EtMgCl (3 M, 435.91 mL) over 30 minutes at 20° C. The mixture was stirred at 70° C. for 12 hrs. The reaction was cooled to 20° C. and a solution of chloro(triisopropyl)silane (114.60 g, 594.42 mmol) in THF (500 mL) was added over a 15 minute period. The mixture was stirred at 70° C. for 12 hours. The reaction mixture was acidified by HCl (1 M) till pH=6, extracted with EtOAc (1500 mL*3). The organic layer was dried over Na₂SO₄, concentrated to give the crude product. The residue was purified by column chromatography on silica gel. 5-triisopropylsilylpent-4-yn-1-ol (94 g, crude) was obtained as a yellow oil.

¹H NMR (400 MHz, CHLOROFORM-d) δ: 3.83-3.77 (m, 2H), 2.44-2.35 (m, 2H), 1.85-1.74 (m, 2H), 1.61-1.58 (m, 1H), 1.08-1.04 (m, 21H)

### Step 2. 5-triisopropylsilylpent-4-ynal

To a solution of (1,1-diacetoxy-3-oxo-1,2-benziodoxol-1-yl) acetate (55.56 g, 131.00 mmol) in DCM (450 mL) was added a solution of 5-triisopropylsilylpent-4-yn-1-ol (30 g, 124.76 mmol) in DCM (20 mL) followed by the addition of H₂O (2.36 g, 131.00 mmol). The reaction was stirred at 20° C. for 24 h. The solution was poured into a 1:1 solution of sodium thiosulfate (300 mL) and sodium bicarbonate (300 mL), extracted with EtOAc (300 mL*3). The organic layer was dried over Na₂SO₄, concentrated to give the crude product which was purified by column chromatography on silica gel. 5-triisopropylsilylpent-4-ynal (20.3 g, crude) was obtained as a yellow oil.

### Step 3. 2-methyl-N-(5-triisopropylsilylpent-4-ynylidene)propane-2-sulfinamide

To a solution of 5-triisopropylsilylpent-4-ynal (20.3 g, 85.14 mmol) in THF (400 mL) was added 2-methylpropane-2-sulfinamide (10.83 g, 89.39 mmol), propan-2-olate;titanium (4+) (48.39 g, 170.27 mmol, 50.67 mL), then the mixture was stirred at 75° C. for 3 h under N2.

The reaction mixture was added to brine (400 mL), then the mixture was filtered to give a solution. The solution was extracted with EtOAc (200 mL*4). The organic layer was dried over Na₂SO₄, concentrated to give 2-methyl-N-(5-triisopropylsilylpent-4-ynylidene)propane-2-sulfinamide (25.2 g, crude) as a white oil.

### Step 4. N-f]-(difluoromethyl)-5-triisopropylsilyl-pent-4-ynyl]-2-methyl-propane-2-sulfinamide

To a solution of 2-methyl-N-(5-triisopropylsilylpent-4-ynylidene)propane-2-sulfinamide (12 g, 11.71 mmol) in THF (60 mL) was added potassium; 2-methylpropan-2-olate (1 M, 33.96 mL), then a solution of difluoromethyl(trimethyl)silane (4.22 g, 33.96 mmol) in THF (20 mL) was added to the above mixture at −70° C. under N2. After addition, the reaction was stirred at 20° C. for 12 h under N2. The reaction mixture was quenched by sat·NH₄Cl (100 mL), extracted with EtOAc (20 mL*3). The organic layer was dried over Na₂SO₄, concentrated to give the crude product which was purified by column chromatography on silica gel. N-[1-(difluoromethyl)-5-triisopropylsilyl-pent-4-ynyl]-2-methyl-propane-2-sulfinamide (2.2 g, 3.81 mmol, 16.27% yield) was obtained as a brown oil.

### Step 5. 1,1-difluoro-6-triisopropylsilyl-hex-5-yn-2-amine

To a solution of N-[1-(difluoromethyl)-5-triisopropylsilyl-pent-4-ynyl]-2-methyl-propane-2-sulfinamide (700 mg, 1.78 mmol) in MeOH (5 mL) was added HCl (4 M, 5.00 mL), then the mixture was stirred at 25° C. for 1 h. The reaction was concentrated under reduced pressure to give 1,1-difluoro-6-triisopropylsilyl-hex-5-yn-2-amine (514 mg, crude) was obtained as a black oil

### Step 6. tert-butyl N-[1]-(difluoromethyl)-5-triisopropylsilyl-pent-4-ynyl]carbamate

To a solution of 1,1-difluoro-6-triisopropylsilyl-hex-5-yn-2-amine (514 mg, 1.78 mmol) in DCM (10 mL) was added TEA (359.35 mg, 3.55 mmol, 494.97 μL) and tert-butoxycarbonyl tert-butyl carbonate (581.28 mg, 2.66 mmol, 611.23 μL), then the mixture was stirred at 25° C. for 12 h. The reaction was concentrated in vacuum. The residue was purified by column chromatography on silica gel to give tert-butyl N-[1-(difluoromethyl)-5-triisopropylsilyl-pent-4-ynyl]carbamate (400 mg, 1.03 mmol, 57.82% yield) as a black oil.

### Step 7. tert-butyl N-allyl-N-[1]-(difluoromethyl)-5-triisopropylsilyl-pent-4-ynyl]carbamate

To a mixture of tert-butyl N-[1-(difluoromethyl)-5-triisopropylsilyl-pent-4-ynyl]carbamate (200 mg, 513.35 μmol) in DMF (10 mL) was added NaH (41.06 mg, 1.03 mmol, 60% purity) at 0° C., and then the mixture was stirred at 25° C. for 0.5 h, and then to the mixture was added 3-iodoprop-1-ene (103.48 mg, 616.03 mol, 56.33 μL), after addition, the mixture was stirred at 25° C. for 1 h. The mixture was quenched by sat. aq. NH₄Cl (20 mL), and then extracted with EtOAc(5 mL*3), and then the organic phase was concentrated to give a residue which was purified by flash chromatography on silica gel to give tert-butyl N-allyl-N-[1-(difluoromethyl)-5-triisopropylsilyl-pent-4-ynyl]carbamate (150 mg, 349.12 μmol, 68.01% yield) as yellow oil

### Step 8. tert-butyl N-allyl-N-[1-(difluoromethyl)pent-4-ynyl]carbamate

To a mixture of tert-butyl N-allyl-N-[1-(difluoromethyl)-5-triisopropylsilyl-pent-4-ynyl]carbamate (600 mg, 1.40 mmol) in THF (10 mL) was added TBAF (1 M, 1.82 mL), and then the mixture was stirred at 25° C. for 12 h. The mixture was concentrated to give a residue which was purified by prep-TLC to give tert-butyl N-allyl-N-[1-(difluoromethyl)pent-4-ynyl]carbamate (380 mg, 1.39 mmol, 99.56% yield) as a yellow oil.

### Step 9. tert-butyl N-allyl-N-[1]-(difluoromethyl)-4-(4,4,5,5-tetramethyl-1,3,2-dioxaborolan-2-yl)pent-4-enyl]carbamate

A mixture of LiCl (13.03 mg, 307.33 μmol, 6.30 μL) and CuCl (30.43 mg, 307.33 mol) in DMF (1 mL) was stirred at 25° C. for 1 hrs under N2, then to the solution was added tert-butyl N-allyl-N-[1-(difluoromethyl)pent-4-ynyl]carbamate (70 mg, 256.11 μmol), 4,4,5,5-tetramethyl-2-(4,4,5,5-tetramethyl-1,3,2-dioxaborolan-2-yl)-1,3,2-dioxaborolane (78.04 mg, 307.33 μmol) and KOAc (86.70 mg, 307.33 μmol) in DMF (1 mL) and the reaction mixture stirred at 25° C. for 12 hrs under N2. The reaction was filtered and added to sat. aq. NH₄Cl (20 mL), extracted with EtOAc (20 mL*5). The organic layer was dried over Na₂SO₄, concentrated to give the crude product. The residue was purified by prep-TLC to give tert-butyl N-allyl-N-[1-(difluoromethyl)-4-(4,4,5,5-tetramethyl-1,3,2-dioxaborolan-2-yl)pent-4-enyl]carbamate (100 mg, 249.19 μmol, 97.30% yield) as a yellow oil.

### Step 10. tert-butyl 2-(difluoromethyl)-5-(4,4,5,5-tetramethyl-1,3,2-dioxaborolan-2-yl)-2,3,4,7-tetrahydroazepine-1-carboxylate

To a mixture of tert-butyl N-allyl-N-[1-(difluoromethyl)-4-(4,4,5,5-tetramethyl-1,3,2-dioxaborolan-2-yl)pent-4-enyl]carbamate (100 mg, 249.19 μmol) was added 2nd-Hoveyda-Grubbs (10 mg), and then the mixture was stirred at 25° C. for 24 h. The residue was purified by prep-TLC to give tert-butyl 2-(difluoromethyl)-5-(4,4,5,5-tetramethyl-1,3,2-dioxaborolan-2-yl)-2,3,4,7-tetrahydroazepine-1-carboxylate (20 mg, 53.58 μmol, 21.50% yield) as a colourless oil.

### Step 11. tert-butyl 2-(difluoromethyl)-5-[6-fluoro-5-[[4-methyl-6-(methylamino)pyrimidin-2-yl]amino]-2,3-dihydrobenzofuran-7-yl]-1-2,3,4,7-tetrahydroazepine-1-carboxylate

To the mixture of tert-butyl 2-(difluoromethyl)-5-(4,4,5,5-tetramethyl-1,3,2-dioxaborolan-2-yl)-2,3,4,7-tetrahydroazepine-1-carboxylate (20 mg, 53.58 μmol) and N2-(7-bromo-6-fluoro-2,3-dihydrobenzofuran-5-yl)-N4,6-dimethyl-pyrimidine-2,4-diamine (18.93 mg, 53.58 μmol) in dioxane (2 mL) and H₂O (0.2 mL) was added K₂CO₃ (22.22 mg, 160.75 μmol) and cyclopentyl(diphenyl)phosphane;dichloropalladium;iron (7.84 mg, 10.72 μmol). The reaction mixture was stirred at 100° C. for 12 hrs. The reaction was concentrated under reduced pressure to give a residue. The residue was dissolved in DCM (10 mL), and then filtered, the filtrate was concentrated to give tert-butyl 2-(difluoromethyl)-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (27 mg, crude) as a black oil.

### Step 12. N2-[6-fluoro-7-[rel-(2R)-2-(difluoromethyl)-2, 3, 4, 7-tetrahydro-1H-azepin-5-yl]-2,3-dihydrobenzofuran-5-yl]-N4,6-dimethyl-pyrimidine-2,4-diamine and N2-[6-fluoro-7-[rel-(2S)-2-(difluoromethyl)-2,3,4,7-tetrahydro-1H-azepin-5-yl]-2,3-dihydrobenzo furan-5-yl]-N4,6-dimethyl-pyrimidine-2,4-diamine

To the solution of tert-butyl 2-(difluoromethyl)-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (27 mg, 51.97 mol) in DCM (5 mL) was added ZnBr₂ (58.51 mg, 259.84 mol), then stirred at 25° C. for 2 hrs. The reaction was filtered and concentrated under reduced pressure to give a residue which was purified by prep-HPLC(TFA condition:column:Phenomenex Luna 80*30 mm*3 um; mobile phase:water(TFA)-ACN; B %: 1%-30%,8 min) to give the mixture of two single isomer. The mixture was separated by Chiral SFC (column: DAICEL CHIRALPAK IE(250 mm*30 mm,10 um); mobile phase: A: CO2 B:MeOH(0.1% IPAm, v/v; SFC; Acq Method: IE_H_I_ACN_IPAm_30_13_30) to give P1 (t=1.690 min) (SFC Method: IE_H_I_ACN_IPAm_30_13_30) and P2 (t=1.938 min) (SFC Method: IE_H_I_ACN_IPAm_30_13_30).

N2-[6-fluoro-7-[rel-(2R)-2-(difluoromethyl)-2,3,4,7-tetrahydro-1H-azepin-5-yl]-2,3-dihydrobenzofuran-5-yl]-N4,6-dimethyl-pyrimidine-2,4-diamine (2.9 mg, 6.91 μmol, 13.30% yield) was obtained as brown solid.

N2-[6-fluoro-7-[rel-(2S)-2-(difluoromethyl)-2,3,4,7-tetrahydro-1H-azepin-5-yl]-2,3-dihydrobenzofuran-5-yl]-N4,6-dimethyl-pyrimidine-2,4-diamine (3.0 mg, 7.15 μmol, 13.76% yield) was obtained as brown solid.

¹H NMR (400 MHz, METHANOL-d₄) δ: 7.80-7.74 (m, 1H), 5.99-5.90 (m, 1H), 5.81 (s, 1H), 5.82-5.81 (m, 1H), 5.80-5.63 (m, 1H), 4.56 (t, J=9.1 Hz, 2H), 3.68-3.57 (m, 1H), 3.47-3.38 (m, 1H), 3.23-3.12 (m, 3H), 2.86 (s, 3H), 2.63 (br t, J=5.1 Hz, 2H), 2.17 (s, 3H), 2.03-1.77 (m, 2H), 0.95-0.95 (m, 1H)

¹H NMR (400 MHz, METHANOL-d₄) δ: 7.73 (br d, J=7.9 Hz, 1H), 5.96 (br t, J=5.5 Hz, 1H), 4.60-4.53 (m, 2H), 3.66-3.59 (m, 1H), 3.47-3.39 (m, 1H), 3.25-3.14 (m, 3H), 2.87 (s, 3H), 2.67-2.59 (m, 2H), 2.21-2.14 (m, 3H), 2.07-1.75 (m, 2H)

### EXAMPLE 68. Synthesis of Compound 360, Compound 355, Compound 419 and Compound 418

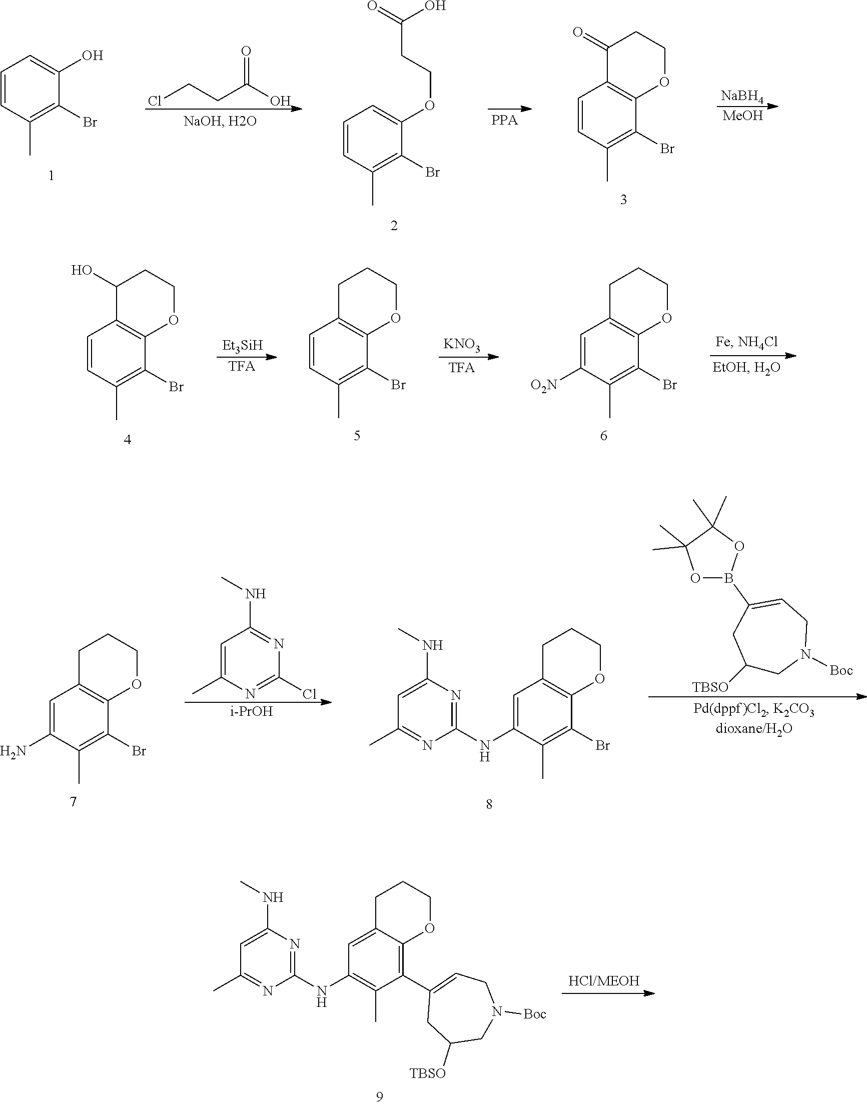

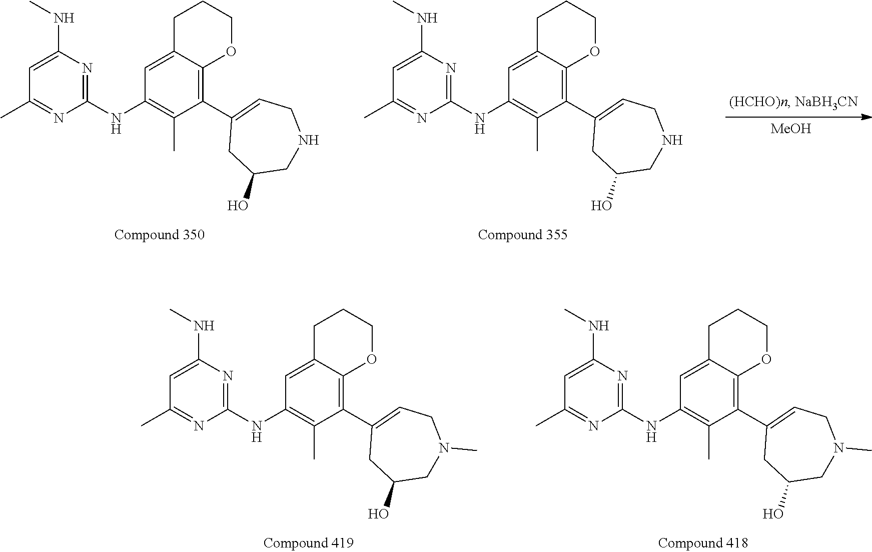

### Step 1. 3-(2-bromo-3-methyl-phenoxy)propanoic acid

A mixture of 2-bromo-3-methyl-phenol (14 g, 74.85 mmol) and 3-chloropropanoic acid (8.12 g, 74.85 mmol, 6.40 mL), then was added dropwise a solution of NaOH (7.49 g, 187.13 mmol, 3.51 mL) in H₂O (45 mL) at 20° C., then the mixture was stirred at 100° C. for 12 h. The reaction was added NH₄Cl, then extracted with ethyl acetate (100 mL*3) and dried over anhydrous sodium sulfate, filtered and concentrated under reduced pressure to give a residue. The residue was added ethyl acetate (50 mL), then filtered and the filter cake concentrated under reduced pressure to give 3-(2-bromo-3-methyl-phenoxy) propanoic acid (5.75 g, 22.19 mmol, 29.65% yield) as a white solid.

### Step 2. 8-bromo-7-methyl-chroman-4-one

A solution of 3-(2-bromo-3-methyl-phenoxy) propanoic acid (5.75 g, 22.19 mmol) in PPA (60 mL), then the mixture was stirred at 100° C. for 2 h. The reaction was added into water (400 mL), then extracted with ethyl acetate (400 mL*3). The combined organic layers were washed with saturated sodium bicarbonate solution (500 mL*3) and dried over anhydrous sodium sulfate, filtered and concentrated under reduced pressure to give a residue. The residue was purified by flash column chromatography on silica gel to give 8-bromo-7-methyl-chroman-4-one (4.87 g, 20.20 mmol, 91.02% yield) as an orange solid.

¹H NMR (400 MHz, METHANOL-d₄) δ: 2.44 (s, 3H) 2.78-2.84 (m, 2H) 4.60-4.66 (m, 2H) 6.99 (d, J=8.11 Hz, 1H) 7.71 (d, J=7.99 Hz, 1H)

### Step 3. 8-bromo-7-methyl-chroman-4-ol

To a solution of 8-bromo-7-methyl-chroman-4-one (4.87 g, 20.20 mmol) in MeOH (50 mL) was added NaBH₄ (1.15 g, 30.30 mmol) at 0° C., then the mixture was stirred at 20° C. for 1 h. The reaction was concentrated under reduced pressure, then added to water (50 mL) and extracted with ethyl acetate (50 mL*3), the organic layer was dried over anhydrous sodium sulfate, filtered and concentrated under reduced pressure to give 8-bromo-7-methyl-chroman-4-ol (4.9 g, crude) as a green solid.

¹H NMR (400 MHz, METHANOL-d₄) δ 1.95-2.02 (m, 1H) 2.03-2.12 (m, 1H) 2.35 (s, 3H) 4.25-4.38 (m, 2H) 4.72 (t, J=4.29 Hz, 1H) 6.84 (d, J=7.87 Hz, 1H) 7.19 (d, J 7.75 Hz, 1H)

### Step 4. 8-bromo-7-methyl-chromane

To a solution of 8-bromo-7-methyl-chroman-4-ol (4.9 g, 20.16 mmol) in TFA (50 mL) was added triethylsilane (4.69 g, 40.31 mmol, 6.44 mL), then the mixture was stirred at 60° C. for 12 h. The reaction was concentrated under reduced pressure, then added water (50 mL), then extracted with ethyl acetate (50 mL*3). The combined organic layers were washed with brine (50 mL*3) and dried over anhydrous sodium sulfate, filtered and concentrated under reduced pressure to give a residue. The residue was purified by column chromatography to give 8-bromo-7-methyl-chromane (2.56 g, 11.27 mmol, 55.93% yield) as a colorless oil.

¹H NMR (400 MHz, METHANOL-d₄) δ: 1.90-1.98 (m, 2H) 2.25 (s, 3H) 2.72 (t, J=6.44 Hz, 2H)4.01-4.16 (m, 2H)6.64 (s, 1H)7.16 (s, 1H).

### Step 5. 8-bromo-7-methyl-6-nitro-chromane

To a solution of 8-bromo-7-methyl-chromane (500 mg, 2.20 mmol) in TFA (5 mL) was added dropwise a solution of KNO₃ (244.86 mg, 2.42 mmol) in TFA (2 mL) at 0° C., then the mixture was stirred at 20° C. for 1 h. The reaction was added into water (15 mL) at 0° C., then extracted with ethyl acetate (20 mL*3). The combined organic layers were washed with saturated sodium bicarbonate solution (50 mL*3) and dried over anhydrous sodium sulfate, filtered and concentrated under reduced pressure to give a residue. The residue was purified by column chromatography on silica gel to give 8-bromo-7-methyl-6-nitro-chromane (380 mg, crude) as a yellow oil.

¹H NMR (400 MHz, METHANOL-d₄) δ: 2.00-2.04 (m, 2H) 2.56 (s, 3H) 2.86 (t, J=6.38 Hz, 2H) 4.34-4.40 (m, 2H) 7.70 (s, 1H).

### Step 6. 8-bromo-7-methyl-chroman-6-amine

To a solution of 8-bromo-7-methyl-6-nitro-chromane (380 mg, 1.40 mmol) and NH₄Cl (747.04 mg, 13.97 mmol) in EtOH (4 mL) and H₂O (0.4 mL) was added Fe (389.96 mg, 6.98 mmol), then the mixture was stirred at 80° C. for 12 h. The reaction was filtered and concentrated under reduced pressure, then added water (10 mL), then extracted with ethyl acetate (10 mL*3) and dried over anhydrous sodium sulfate, filtered and concentrated under reduced pressure to give a residue. The residue was purified by prep-TLC to give 8-bromo-7-methyl-chroman-6-amine (170 mg, 702.15 μmol, 50.28% yield) as a yellow oil.

### Step 7. N2-(8-bromo-7-methyl-chroman-6-yl)-N4,6-dimethyl-pyrimidine-2,4-diamine

To a solution of 8-bromo-7-methyl-chroman-6-amine (118.07 mg, 487.65 mol) and 2-chloro-N,6-dimethyl-pyrimidin-4-amine (76.85 mg, 487.65 mol) in i-PrOH (3.00 mL) was added TFA (5.56 mg, 48.77 mol, 3.73 L), then the mixture was stirred at 140° C. for 2 hrs. The reaction was concentrated under reduced pressure to give N2-(8-bromo-7-methyl-chroman-6-yl)-N4,6-dimethyl-pyrimidine-2,4-diamine (170 mg, crude) as brown solid.

### Step 8. tert-butyl 3-[tert-butyl(dimethyl)silyl]oxy-5-[7-methyl-6-[[4-methyl-6-(methylamino)pyrimidin-2-yl]amino]chroman-8-yl]-2,3,4,7-tetrahydroazepine-1-carboxylate

To a mixture of N2-(8-bromo-7-methyl-chroman-6-yl)-N4,6-dimethyl-pyrimidine-2,4-diamine (170 mg, 468.00 mol) and tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (212.23 mg, 468.00 μmol) in dioxane (8 mL) and H₂O (0.8 mL) was added K₂CO₃ (129.36 mg, 935.99 μmol) and cyclopentyl(diphenyl)phosphane;dichloromethane;dichloropalladium;iron (38.22 mg, 46.80 μmol) under N₂ atmosphere. The reaction mixture was stirred at 100° C. for 12 hrs under N₂. The reaction was filtered and concentrated under reduced pressure to give tert-butyl 3-[tert-butyl(dimethyl)silyl]oxy-5-[7-methyl-6-[[4-methyl-6-(methylamino)pyrimidin-2-yl]amino]chroman-8-yl]-2,3,4,7-tetrahydroazepine-1-carboxylate(100 mg, crude) as a brown oil.

### Step 9.rel-(3S)-5-[7-methyl-6-[[4-methyl-6-(methylamino)pyrimidin-2-yl]amino]chroman-8-yl]-2,3,4,7-tetrahydro-1H-azepin-3-ol and rel-(3R)-5-[7-methyl-6-[[4-methyl-6-(methylamino)pyrimidin-2-yl]amino]chroman-8-yl]-2,3,4,7-tetrahydro-1H-azepin-3-ol

A solution of tert-butyl 3-[tert-butyl(dimethyl)silyl]oxy-5-[7-methyl-6-[[4-methyl-6-(methylamino)pyrimidin-2-yl]amino]chroman-8-yl]-2,3,4,7-tetrahydroazepine-1-carboxylate (350 mg, 573.89 mol) in HCl/MeOH (12 mL) was stirred at 20° C. for 2 hrs. The reaction mixture was concentrated and purified by prep-HPLC (TFA condition:column: Phenomenex Luna 80*30 mm*3 um; mobile phase:water(TFA)-ACN; B %: 1%-25%,8 min) to give the mixture of these two single isomer. The mixture was separated by SFC (column: DAICEL CHIRALPAKIC (250 mm*30 mm,10 um); mobile phase:[0.1% NH₃H₂O MEOH]; B %: 50%-55%; SFC, Acq Method: IC_MeOH_IPAm_5_50_3_35_3 min) to give rel-(3S)-5-[7-methyl-6-[[4-methyl-6-(methylamino)pyrimidin-2-yl]amino]chroman-8-yl]-2,3,4,7-tetrahydro-1H-azepin-3-ol (3 mg, 7.59 μmol, 1.32% yield, Rt=4.289 min) as a yellow solid and rel-(3R)-5-[7-methyl-6-[[4-methyl-6-(methylamino)pyrimidin-2-yl]amino]chroman-8-yl]-2,3,4,7-tetrahydro-1H-azepin-3-ol (1 mg, 2.53 μmol, 4.41e-1% yield, Rt=5.434 min) as a pale yellow solid.

¹HNMR (400 MHz, METHANOL-d₄) δ: 1.97-2.06 (m, 2H) 2.13-2.22 (m, 3H) 2.26 (br s, 4H)2.75-2.87 (m, 4H)2.89-2.94 (m, 3H)3.33 (br s, 11H)3.38-3.74 (m, 3H)3.83-3.95 (m, 2H) 4.23 (br s, 2H) 4.26-4.32 (m, 1H) 4.88 (s, 16H) 5.67-5.75 (m, 1H) 5.95 (br s, 1H) 7.01-7.10 (m, 1H).

¹HNMR (400 MHz, METHANOL-d₄) δ:1.97-2.07 (m, 2H) 2.26 (br s, 2H) 2.14-2.22 (m, 3H) 2.68-2.97 (m, 4H) 2.92 (s, 4H) 3.33 (dt, J=3.19, 1.56 Hz, 1H) 3.46-3.66 (m, 2H) 3.86-3.96 (m, 2H) 4.19-4.31 (m, 3H) 4.89 (s, 1H) 5.67-5.76 (m, 1H) 5.95 (br s, 1H) 7.05 (br s, 1H)

### Step 10. rel-(3S)-1-methyl-5-[7-methyl-6-[[4-methyl-6-(methylamino)pyrimidin-2-yl]amino]chroman-8-yl]-2,3,4,7-tetrahydroazepin-3-ol and rel-(3R)-1-methyl-5-[7-methyl-6-[[4-methyl-6-(methylamino)pyrimidin-2-yl]amino]chroman-8-yl]-2,3,4,7-tetrahydroazepin-3-ol

To a solution rel-(3S)-5-[7-methyl-6-[[4-methyl-6-(methylamino)pyrimidin-2-yl]amino]chroman-8-yl]-2,3,4,7-tetrahydro-1H-azepin-3-ol (10 mg, 25.28 μmol) in MeOH (2 mL) was added formaldehyde (759.19 μg, 25.28 μmol, 7.01e-1 μL), then the mixture was added sodium; cyanoboranuide (4.77 mg, 75.85 mol) and stirred at 25° C. for 12 h. The reaction was filtered and concentrated under reduced pressure to give a residue. The crude product was purified by prep-HPLC(TFA condition:column: Phenomenex Luna 80*30 mm*3 um; mobile phase:water(TFA)-ACN; B %: 1%-30%,8 min) to give rel-(3S)-1-methyl-5-[7-methyl-6-[[4-methyl-6-(methylamino)pyrimidin-2-yl]amino]chroman-8-yl]-2,3,4,7-tetrahydroazepin-3-ol (6.2 mg, 15.14 μmol, 59.88% yield, t=5.162) was obtained as a yellow oil.

¹H NMR (400 MHz, METHANOL-d₄) δ: 2.00 (br d, J=4.28 Hz, 2H) 2.10-2.20 (m, 3H) 2.24 (s, 3H) 2.36 (s, 1H) 2.80 (br t, J=6.11 Hz, 3H) 2.83-2.91 (m, 4H) 2.99 (br s, 4H) 4.09 (br dd, J=13.63, 6.17 Hz, 1H) 4.22 (br s, 2H) 4.27-4.35 (m, 1H) 5.57-5.73 (m, 1H) 5.93 (s, 1H) 7.04 (s, 1H)

To a mixture of rel-(3R)-5-[7-methyl-6-[[4-methyl-6-(methylamino)pyrimidin-2-yl]amino]chroman-8-yl]-2,3,4,7-tetrahydro-1H-azepin-3-ol (9.58 mg, 24.22 μmol) and formaldehyde (1.45 mg, 48.45 mol, 1.34 μL) in MeOH (5 mL) was added sodium; cyanoboranuide (4.57 mg, 72.67 μmol), then stirred at 25° C. for 12 hrs. The reaction mixture was filtered and purified by prep-HPLC; TFA condition:column: Phenomenex Luna 80*30 mm*3 um; mobile phase:water(TFA)-ACN; B %: 1%-30%,8 min) to give a residue. concentrated to give a rel-(3R)-1-methyl-5-[7-methyl-6-[[4-methyl-6-(methylamino) pyrimidin-2-yl]amino]chroman-8-yl]-2,3,4,7-tetrahydroazepin-3-ol (7.2 mg, 17.58 μmol, 72.58% yield, Rt=4.213 min) was obtained as a white solid.

¹H NMR (400 MHz, METHANOL-d₄) δ: 1.35-1.44 (m, 1H) 1.95-2.08 (m, 2H) 2.13-2.22 (m, 3H) 2.26 (s, 3H) 2.33-2.44 (m, 1H) 2.82 (br t, J=6.32 Hz, 3H) 2.88-2.94 (m, 4H) 2.97-3.06 (m, 4H) 3.33 (dt, J=3.19, 1.56 Hz, 7H) 4.11 (br dd, J=14.31, 6.20 Hz, 1H) 4.24 (br s,2 H) 4.29-4.37 (m, 1H) 4.88 (s, 19H) 5.57-5.77 (m, 1H) 5.95 (s, 1H) 6.97-7.02 (m, 1H) 6.98-7.01 (m, 1H) 7.06 (s, 1H)

### EXAMPLE 69. Synthesis of Compound 357 and Compound 365

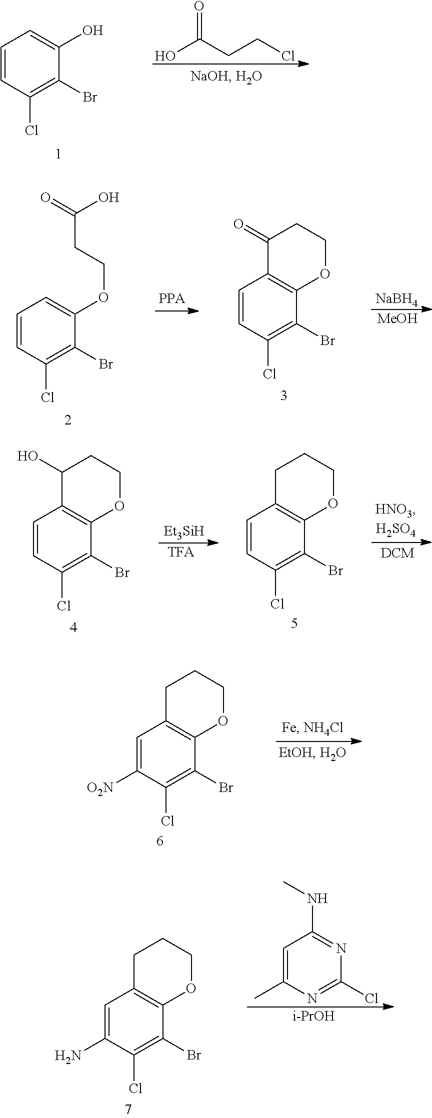

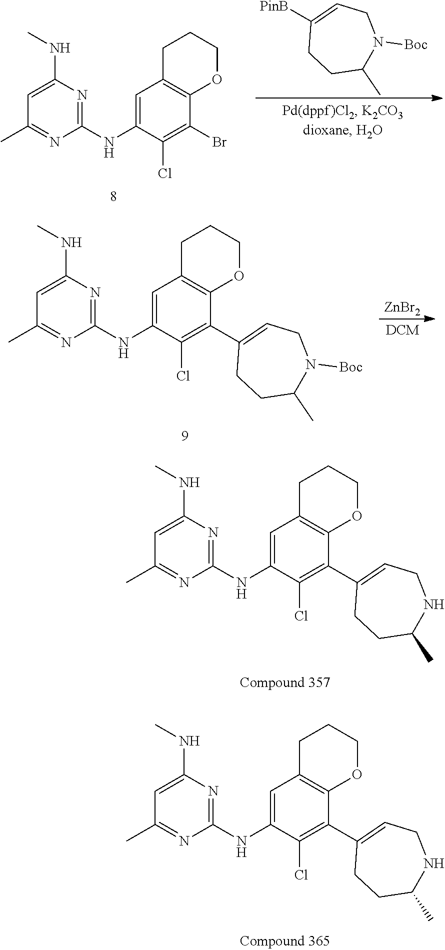

### Step 1. 3-(2-bromo-3-chloro-phenoxy)propanoic acid

2-bromo-3-chloro-phenol (10 g, 48.20 mmol) and 3-chloropropanoic acid (5.23 g, 48.20 mmol, 4.12 mL) were taken in a conical flask to which aqueous solution of NaOH (4.63 g, 115.69 mmol, 2.17 mL) in H₂O (100 mL) was slowly added with constant stirring. The solution was stirred for 12 hrs at 100° C. The reaction was added (1N HCl) to pH=2, then extracted with ethyl acetate (100 mL*3) and dried over anhydrous sodium sulfate, filtered and concentrated under reduced pressure to give a residue. The residue was purified by column chromatography on silica gel to give 3-(2-bromo-3-chloro-phenoxy)propanoic acid (4.5 g, crude) as a white solid.

### Step 2. 8-bromo-7-fluoro-chroman-4-one

To a solution of 3-(2-bromo-3-fluoro-phenoxy)propanoic acid (4 g, 15.21 mmol) in PPA (40 mL), then the mixture was stirred at 100° C. for 2 h. The reaction mixture was quenched by water (200 mL) and extracted with EtOAc (100 mL*3) and dried over anhydrous sodium sulfate, filtered and concentrated under reduced pressure to give 8-bromo-7-fluoro-chroman-4-one (3.7 g, crude) as a yellow solid.

### Step 3. 8-bromo-7-chloro-chroman-4-ol

To a solution of 8-bromo-7-chloro-chroman-4-one (900 mg, 3.44 mmol) in MeOH (10 mL) was added NaBH₄ (195.31 mg, 5.16 mmol) at 0° C., then the mixture was stirred at 20° C. for 1 h. The reaction was concentrated under reduced pressure. The residue was purified by flash chromatography on silica gel. 8-bromo-7-chloro-chroman-4-ol (900 mg, 3.42 mmol, 99.24% yield) was obtained as a yellow oil.

### Step 4. 8-bromo-7-chloro-chromane

To a solution of 8-bromo-7-chloro-chroman-4-ol (880 mg, 3.34 mmol) in TFA (10 mL) was added triethylsilane (776.61 mg, 6.68 mmol, 1.07 mL), then the mixture was stirred at 60° C. for 4 hrs. The reaction was added into water (25 mL), then extracted with ethyl acetate (15 ml*3). The combined organic layers were washed with brine (10 mL) and dried over anhydrous sodium sulfate, filtered and concentrated under reduced pressure to give a residue. The residue was purified by column chromatography on silica gel to give 8-bromo-7-chloro-chromane (876 mg, crude) as clear oil.

### Step 5. 8-bromo-7-chloro-6-nitro-chromane

To a solution of 8-bromo-7-chloro-chromane (870 mg, 3.51 mmol) in DCM (10 mL) was added a mixture of nitric acid (232.55 mg, 3.69 mmol, 157.13 μL) and sulfuric acid (1.41 g, 14.06 mmol, 764.74 μL, 98% purity) at 0° C., then the mixture was stirred at 20° C. for 1 h. The reaction was added in water (25 mL) at 0° C., then extracted with dichloromethane(50 mL*3). The combined organic layers were washed with saturated sodium bicarbonate solution (100 mL*3) and dried over anhydrous sodium sulfate, filtered and concentrated under reduced pressure to give a residue which was purified by column chromatography on silica gel to give 8-bromo-7-chloro-6-nitro-chromane (750 mg, crude) as a brown solid.

### Step 6. 8-bromo-7-chloro-chroman-6-amine

To a solution of 8-bromo-7-chloro-6-nitro-chromane (750 mg, 2.56 mmol),NH₄Cl (137.15 mg, 2.56 mmol) in EtOH(10 mL) and H₂O (1 mL) was added Fe(143.19 mg, 2.56 mmol), then the mixture was stirred at 80° C. for 2 hrs. The reaction was filtered and concentrated under reduced pressure to give a residue. The reaction mixture was added to water (10 mL), extracted with EtOAc (10 mL*5). The organic layer was dried over Na₂SO₄, concentrated to give 8-bromo-7-chloro-chroman-6-amine (490 mg, crude) as brown oil.

### Step 7. N2-(8-bromo-7-chloro-chroman-6-yl)-N4,6-dimethyl-pyrimidine-2,4-diamine

A mixture of 2-chloro-N,6-dimethyl-pyrimidin-4-amine (280.95 mg, 1.78 mmol) and 8-bromo-7-chloro-chroman-6-amine (390 mg, 1.49 mmol) in i-PrOH (5 mL) was stirred at 130° C. in the microwave for 2 hrs. The reaction was concentrated under reduced pressure to give N2-(8-bromo-7-chloro-chroman-6-yl)-N4,6-dimethyl-pyrimidine-2,4-diamine (560 mg, crude) as a brown solid.

### Step 8. tert-butyl5-[7-chloro-6-[[4-methyl-6-(methylamino)pyrimidin-2-yl]amino]chroman-8-yl]-2-methyl-2,3,4,7-tetrahydroazepine-1-carboxylate

To a mixture of N2-(8-bromo-7-chloro-chroman-6-yl)-N4,6-dimethyl-pyrimidine-2,4-diamine (80 mg, 208.51 mol) and tert-butyl 2-methyl-5-(4,4,5,5-tetramethyl-1,3,2-dioxaborolan-2-yl)-2,3,4,7-tetrahydroazepine-1-carboxylate (70.32 mg, 208.51 μmol) in H₂O (0.3 mL) and dioxane (3 mL) was added K₂CO₃ (57.64 mg, 417.02 mol) and cyclopentyl(diphenyl)phosphane;dichloromethane;dichloropalladium;iron (17.03 mg, 20.85 mol) under N₂ atmosphere. The reaction mixture was stirred at 100° C. for 12 hrs under N2 atmosphere. The reaction was filtered and concentrated under reduced pressure to give tert-butyl 5-[7-chloro-6-[[4-methyl-6-(methylamino)pyrimidin-2-yl]amino]chroman-8-yl]-2-methyl-2,3,4,7-tetrahydroazepine-1-carboxylate (100 mg, crude) was obtained as a brown oil.

### Step 9. N2-[7-chloro-8-[rel-(2S)-2-methyl-2,3,4,7-tetrahydro-1H-azepin-5-yl]chroman-6-yl-N4,6-dimethyl-pyrimidine-2,4-diamine and N2-[7-chloro-8-[rel-(2R)-2-methyl-2,3,4,7-tetrahydro-1H-azepin-5-yl]chroman-6-yl]-N4,6-dimethyl-pyrimidine-2,4-diamine

To a solution of tert-butyl 5-[7-chloro-6-[[4-methyl-6-(methylamino)pyrimidin-2-yl]amino]chroman-8-yl]-2-methyl-2,3,4,7-tetrahydroazepine-1-carboxylate (100 mg, 194.53 mol) was added ZnBr₂ (219.04 mg, 972.65 μmol) ZnBr₂ (219.04 mg, 972.65 mol) stirred at 30° C. for 4 hrs. The reaction was concentrated under reduced pressure to give a residue. The residue was purified by prep-HPLC (TFA condition, column: Phenomenex Luna 75*30 mm*3 um; mobile phase: [water (TFA)-ACN; B %: 1%-30%, 8 min) to give crude product. The crude product was purified by SFC (Instrument: Waters SFC80 preparative SFC; Column: DAICEL CHIRALPAK IG(250 mm*30 mm,10 um); Mobile phase: A for CO₂ and B for IPA(0.1% NH₃H₂O); Gradient: B %=40% isocratic elution mode;) to give N2-[7-chloro-8-[rel-(2S)-2-methyl-2,3,4,7-tetrahydro-1H-azepin-5-yl]chroman-6-yl]-N4,6-dimethyl-pyrimidine-2,4-diamine (10.2 mg, 24.64 μmol, 12.67% yield) as a pale yellow solid and N2-[7-chloro-8-[rel-(2R)-2-methyl-2,3,4,7-tetrahydro-1H-azepin-5-yl]chroman-6-yl]-N4,6-dimethyl-pyrimidine-2,4-diamine (16.8 mg, 40.59 μmol, 20.86% yield) as a pale yellow solid.

¹H NMR (400 MHz, CD₃OD, 298 K) δ: 8.01 (br s, 1H), 5.83 (s, 1H), 5.73-5.62 (m, 1H), 4.26-4.08 (m, 2H), 3.63-3.37 (m, 2H), 3.18-3.00 (m, 1H), 2.95-2.87 (m, 3H), 2.86-2.76 (m, 2H), 2.67-2.44 (m, 1H), 2.38-2.25 (m, 1H), 2.18 (s, 3H), 2.07-1.88 (m, 3H), 1.80-1.60 (m, 1H), 1.26-1.13 (m, 3H)

¹H NMR (400 MHz, CD₃OD, 299 K) δ: 7.38 (br d, J=4.8 Hz, 1H), 5.99 (s, 1H), 5.79-5.69 (m, 1H), 4.28-4.23 (m, 1H), 4.22-4.16 (m, 1H), 3.93-3.85 (m, 2H), 3.84-3.84 (m, 1H), 3.66-3.53 (m, 1H), 2.97-2.88 (m, 3H), 2.86-2.72 (m, 3H), 2.48-2.37 (m, 1H), 2.27 (s, 3H), 2.14-1.90 (m, 4H), 1.41 (d, J=6.5 Hz, 3H)

### EXAMPLE 70. Synthesis of Compound 370, Compound 358, Compound 375 and Compound 379

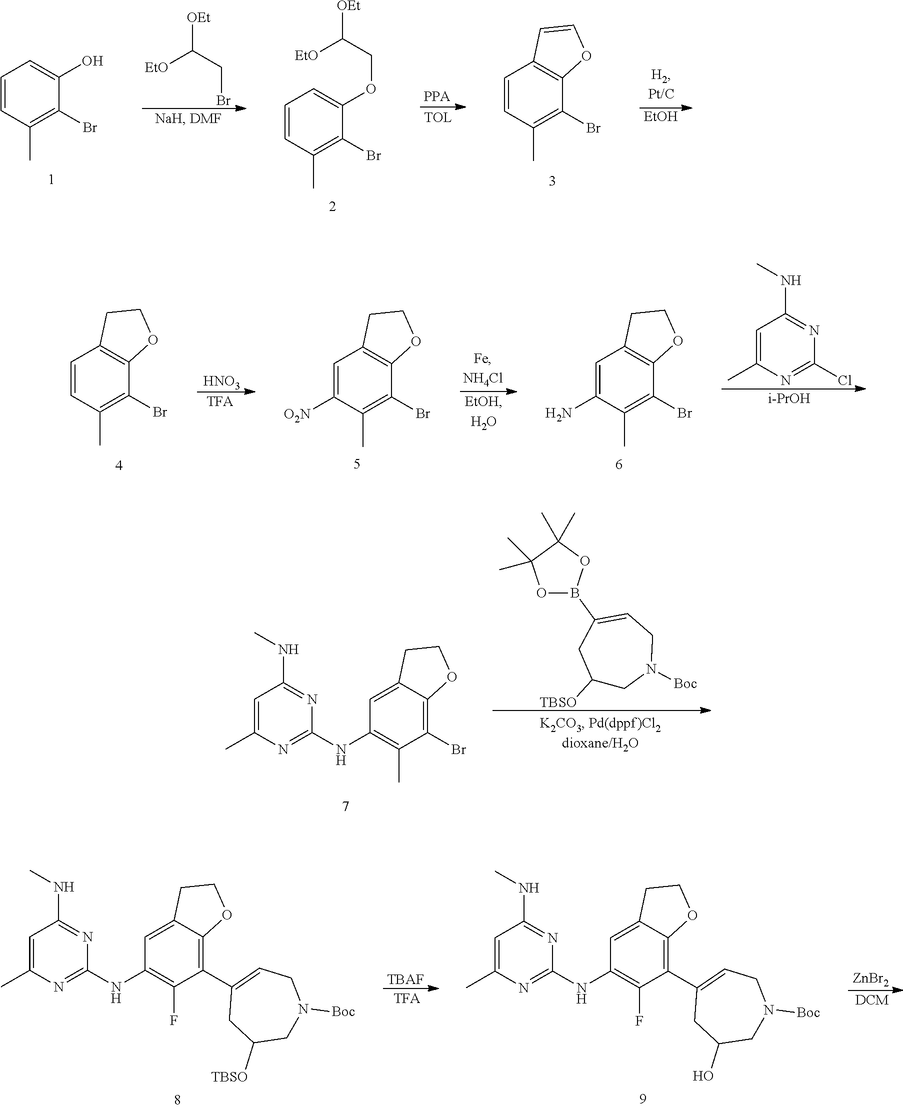

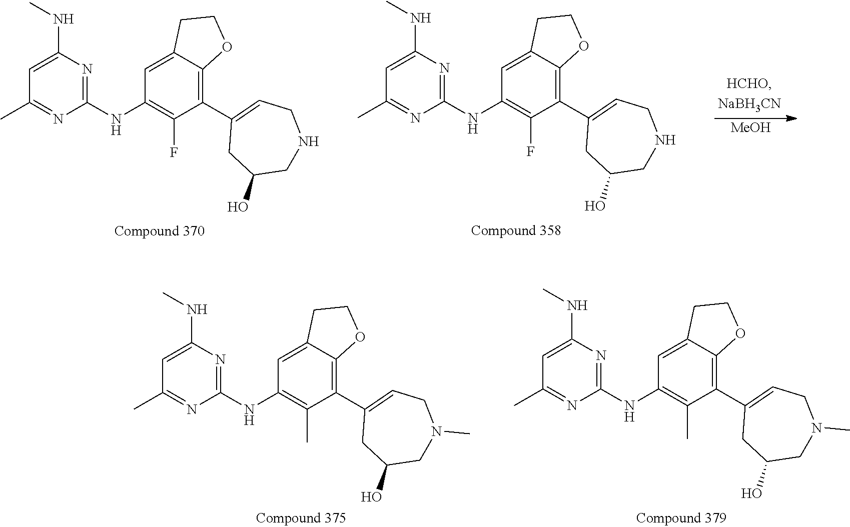

Step 1. 2-bromo-1-(2,2-diethoxyethoxy)-3-methyl-benzene

To a solution of 2-bromo-3-methyl-phenol (5 g, 26.73 mmol) in DMF (50 mL) was added NaH (1.60 g, 40.10 mmol, 784.66 μL, 60% purity) at 0° C. The reaction mixture was stirred at 0° C. for 0.5 h. Then 2-bromo-1,1-diethoxy-ethane (6.32 g, 32.08 mmol) was added to above mixture at 0° C. and stirred at 80° C. for 12 h. The reaction was added into sat. aq. NH₄Cl (50 mL), extracted with ethyl acetate (100 mL*3). The combined organic layers were washed with brine (50 mL*3) and dried over anhydrous sodium sulfate, filtered and the filtrate was concentrated under reduced pressure to give a residue. The residue was purified by flash chromatography to give 2-bromo-1-(2,2-diethoxyethoxy)-3-methyl-benzene (7 g, crude) as yellow oil.

### Step 2. 7-bromo-6-methyl-benzofuran

2-bromo-1-(2,2-diethoxyethoxy)-3-methyl-benzene (7 g, 23.09 mmol) was dissolved in toluene (220 mL), PPA (15 g, 23.09 mmol) was added to the solution, then the mixture was stirred at 110° C. for 2 h. The mixture was concentrated in vacuo. The residue was purified by flash chromatography to give 7-bromo-6-methyl-benzofuran (1.8 g, crude) as a yellow oil.

### Step 3. 7-bromo-6-methyl-2,3-dihydrobenzofuran

To the mixture of 7-bromo-6-methyl-benzofuran (1.8 g, 8.53 mmol) in EtOH (180 mL) was added Pt\C (179.19 mg, 852.86 mol). The reaction mixture was stirred at 25° C. for 12 hrs under H₂ (15 psi). The mixture was filtered and the filtrate was concentrated in vacuum. The residue was purified by flash chromatography to give 7-bromo-6-methyl-2,3-dihydrobenzofuran (1.3 g, crude) as a yellow oil.

¹H NMR (400 MHz, CHLOROFORM-d) δ: 2.22 (s, 1H) 2.28 (s, 3H) 3.21 (t, J=8.69 Hz, 2H) 4.58 (t, J=8.76 Hz, 2H) 6.65 (d, J=7.50 Hz, 1H) 6.92 (d, J=7.38 Hz, 1H) 7.18 (s, 1 H)

### Step 4. 7-bromo-6-methyl-5-nitro-2,3-dihydrobenzofuran

HNO₃ (848.07 mg, 9.15 mmol, 68% purity) was added dropwise to the mixture of 7-bromo-6-methyl-2,3-dihydrobenzofuran (1.3 g, 6.10 mmol) in TFA (14 mL) at 0° C.-10° C. After addition, the mixture was stirred at 25° C. for 1 h. The suspension was added to ice (200 mL), and then the suspension was filtered, and the filter cake was washed with H₂O (30 mL*3), and then concentrated in vacuum. 7-bromo-6-methyl-5-nitro-2,3-dihydrobenzofuran (1 g, 3.87 mmol, 63.510% yield) was obtained as a yellow solid.

¹H NMR (400 MHz, CHLOROFORM-d) δ: 1.47 (s, 1H) 2.56 (s, 3H) 3.31 (t, J=8.82 Hz, 2H) 4.73 (t, J=8.82 Hz, 2H) 7.19 (s, 1H) 7.70 (s, 1H)

### Step 5. 7-bromo-6-methyl-5-nitro-2,3-dihydrobenzofuran

To a mixture of 7-bromo-6-methyl-5-nitro-2,3-dihydrobenzofuran (1 g, 3.87 mmol) in EtOH (20 mL), H₂O (2 mL) was added Fe (1.08 g, 19.37 mmol), NH₄Cl (2.07 g, 38.75 mmol) at 25° C., then the mixture was stirred at 80° C. for 12 hrs. The reaction mixture was filtered and concentrated to remove most EtOH and then extracted with EtOAc (10 mL*3). The combined organic layers were washed with 10 mL of brine, dried over Na₂SO₄, filtered and concentrated under reduced pressure to give the crude product. The crude product was purified by prep-TLC to give 7-bromo-6-methyl-2,3-dihydrobenzofuran-5-amine (0.9 g, crude) as a yellow solid.

### Step 6. N2-(7-bromo-6-methyl-2,3-dihydrobenzofuran-5-yl)-N4,6-dimethyl-pyrimidine-2,4-diamine

To a solution of 7-bromo-6-methyl-2,3-dihydrobenzofuran-5-amine (200 mg, 876.86 μmol) and 2-chloro-N,6-dimethyl-pyrimidin-4-amine (138.19 mg, 876.86 μmol) in i-PrOH (4 mL) was added TFA (10 mg), then the mixture was stirred at 140° C. for 2 hrs. The reaction was concentrated under reduced pressure to give N2-(7-bromo-6-methyl-2,3-dihydrobenzofuran-5-yl)-N4,6-dimethyl-pyrimidine-2,4-diamine (240 mg, crude) as brown solid.

### Step 7. tert-butyl2-[tert-butyl(dimethyl)silyl]oxy-5-[6-methyl-5-[{4-methyl-6-(methylamino)-pyrimidin-2-yl]amino]-2,3-dihydrobenzofuran-7-yl]-2,3,4,7-tetrahydroazepine-1-carboxylate

To a mixture of N2-(7-bromo-6-methyl-2,3-dihydrobenzofuran-5-yl)-N4,6-dimethyl-pyrimidine-2,4-diamine (100 mg, 286.35 mol) in H₂O (0.3 mL) and dioxane (3 mL) was added K₂CO₃ (79.15 mg, 572.70 mol) and cyclopentyl (diphenyl) phosphane;dichloromethane dichloropalladium;iron (23.38 mg, 28.63 μmol) under N₂ atmosphere. The reaction mixture was stirred at 100° C. for 12 h under N₂ atmosphere. The reaction was concentrated under reduced pressure to give the mixture under reduced pressure to give tert-butyl2-[tert-butyl(dimethyl)silyl]oxy-5-[6-methyl-5-[[4-methyl-6-(methylamino)-pyrimidin-2-yl]amino]-2,3-dihydrobenzofuran-7-yl]-2,3,4,7-tetrahydroazepine-1-carboxylate (crude) as a brown solid.

### Step 8. tert-butyl 3-hydroxy-5-[6-methyl-5-[[4-methyl-6-(methylamino)pyrimidin-2-yl]amino]-2,3-dihydrobenzofuran-7-yl]-2,3,4,7-tetrahydroazepine-1-carboxylate

To a solution of tert-butyl 3-[tert-butyl(dimethyl)silyl]oxy-5-[6-methyl-5-[[4-methyl-6-(methylamino)pyrimidin-2-yl]amino]-2,3-dihydrobenzofuran-7-yl]-2,3,4,7-tetrahydroazepine-1-carboxylate (0.120 g, 201.39 mol) in THF (3 mL) was added TBAF (1 mL) and then stirred at 20° C. for 12 h. The reaction mixture was added to water (5 mL), extracted with EtOAc (10 mL*5). The organic layer was dried over Na₂SO₄, concentrated to give the crude product. The residue was purified by prep-TLC to give tert-butyl 3-hydroxy-5-[6-methyl-5-[[4-methyl-6-(methylamino)pyrimidin-2-yl]amino]-2,3-dihydrobenzofuran-7-yl]-2,3,4,7-tetrahydroazepine-1-carboxylate (79.5 mg, crude) as yellow solid.

### Step 9. rel-(3S)-5-[6-methyl-5-[[4-methyl-6-(methylamino)pyrimidin-2-yl]amino]-2,3-dihydrobenzofuran-7-yl]-2,3,4,7-tetrahydro-1H-azepin-3-ol and rel-(3R)-5-[6-methyl-5-[[4-methyl-6-(methylamino)pyrimidin-2-yl]amino]-2,3-dihydrobenzofuran-7-yl]-2,3,4,7-tetrahydro-1H-azepin-3-ol

To a mixture of tert-butyl 3-hydroxy-5-[6-methyl-5-[[4-methyl-6-(methylamino)pyrimidin-2-yl]amino]-2,3-dihydrobenzofuran-7-yl]-2,3,4,7-tetrahydroazepine-1-carboxylate (79.50 mg, 165.08 mol) in DCM (3 mL) was added ZnBr₂ (371.76 mg, 1.65 mmol) at 25° C., then the mixture was stirred at 25° C. for 12 hrs. The reaction mixture was filtered and purified by prep-HPLC; TFA condition:column:Phenomenex Luna 80*30 mm*3 um; mobile phase:water(TFA)-ACN; B %: 1%-25%,8 min) to give a residue. The residue was separated by SFC (column: DAICEL CHIRALPAKIC (250 mm*30 mm,10 um); mobile phase: [A for Heptane and B for EtOH (0.1% IPA); Gradient: B %=50-50%;Acq Method:IG_H_E_ACN_IPAm_50_13_30_5 cm) to give rel-(3S)-5-[6-methyl-5-[[4-methyl-6-(methylamino)pyrimidin-2-yl]amino]-2,3-dihydrobenzofuran-7-yl]-2,3,4,7-tetrahydro-1H-azepin-3-ol (15 mg, 39.32 mol, 23.82% yield) was obtained as a white solid. And rel-(3R)-5-[6-methyl-5-[[4-methyl-6-(methylamino)pyrimidin-2-yl]amino]-2,3-dihydrobenzofuran-7-yl]-2,3,4,7-tetrahydro-1H-azepin-3-ol (14 mg, 36.70 μmol, 22.23% yield) as a white solid.

¹H NMR (400 MHz, METHANOL-d₄) δ: 2.15 (s, 6H) 2.49-2.62 (m, 1H) 2.81 (s, 3H) 2.91 (br dd, J=13.32, 8.44 Hz, 1H) 3.19 (s, 2H) 3.29-3.35 (m, 1H) 3.31 (dt, J=3.25, 1.63 Hz, 22H) 3.39-3.54 (m, 2H) 3.89-4.06 (m, 1H) 4.53 (br d, J=1.38 Hz, 1H) 4.86-4.87 (m, 1H) 4.86 (s, 24H) 5.73-5.76 (m, 1H) 5.80 (br s, 1H) 7.25 (s, 1H).

¹H NMR (400 MHz, METHANOL-d₄) δ: 2.14 (s, 6H) 2.51-2.61 (m, 1H) 2.80 (s, 3H) 2.83-2.93 (m, 1H) 3.13-3.22 (m, 2H) 3.38-3.51 (m, 2H) 3.88-4.03 (m, 1H) 4.47-4.56 (m, 2H)5.72-5.74 (m, 1H)5.75-5.82 (m, 1H)7.22-7.28 (m, 1H).

### Step 10. rel-(3S)-1-methyl-5-[6-methyl-5-[[4-methyl-6-(methylamino)pyrimidin-2-yl]amino]-2,3-dihydrobenzo furan-7-yl J-2,3,4,7-tetrahydroazepin-3-ol and rel-(3R)-1-methyl-5-[6-methyl-5-[[4-methyl-6-(methylamino)pyrimidin-2-yl]amino]-2,3-dihydrobenzofuran-7-yl]-2,3,4,7-tetrahydroazepin-3-ol

To a mixture of 5-[6-methyl-5-[[4-methyl-6-(methylamino)pyrimidin-2-yl]amino]-2,3-dihydrobenzofuran-7-yl]-2,3,4,7-tetrahydro-1H-azepin-3-ol(200 mg,524.29 mol) and formaldehyde (15.74 mg, 524.29 mol, 14.54 L) in MeOH (6 mL) was added sodium cyanoboranuide (98.84 mg, 1.57 mmol), then the reaction mixture was stirred at 25° C. for 12 hrs. The reaction mixture was filtered and purified by prep-HPLC (TFA condition: column:Phenomenex Luna 80*30 mm*3 um; mobile phase:water(TFA)-ACN; B %: 1%-30%,8 min) to give a residue. The residue was separated by SFC (column: DAICEL CHIRALPAKIC (250 mm*30 mm,10 um); mobile phase: [A for Heptane and B for EtOH (0.1% IPA); Gradient: B %=50-50%;Acq Method:IG_H_E_ACN_IPAm_50_13_30_5 cm) to give rel-(3S)-1-methyl-5-[6-methyl-5-[[4-methyl-6-(methylamino) pyrimidin-2-yl]amino]-2,3-dihydro benzofuran-7-yl]-2,3,4,7-tetrahydroazepin-3-ol (1 mg, 2.53 mol, 4.82e-1% yield) as a yellow solid and rel-(3R)-1-methyl-5-[6-methyl-5-[[4-methyl-6-(methylamino)pyrimidin-2-yl]amino]-2,3-dihydrobenzofuran-7-yl]-2,3,4,7-tetrahydroazepin-3-ol (2.8 mg, 7.08 μmol, 1.35% yield) as a yellow solid.

¹H NMR (400 MHz, METHANOL-d₄) δ: 1.98-2.09 (m, 6H) 2.29-2.38 (m, 3H) 2.50-2.60 (m, 1H) 2.64-2.76 (m, 4H) 2.99-3.17 (m, 5H) 4.35-4.46 (m, 2H) 5.57-5.62 (m, 1H) 5.63 (s, 1H) 7.06-7.25 (m, 1H).

¹H NMR (400 MHz, METHANOL-d₄) δ: 1.93-2.13 (m, 6H) 2.31-2.42 (m, 4H) 2.49-2.60 (m, 1H) 2.62-2.75 (m, 4H) 2.99-3.18 (m, 5H) 4.36-4.47 (m, 2H) 5.57-5.62 (m, 1H) 5.63 (s, 1H) 7.04-7.22 (m, 1H)

### EXAMPLE 71. Synthesis of Compound 359

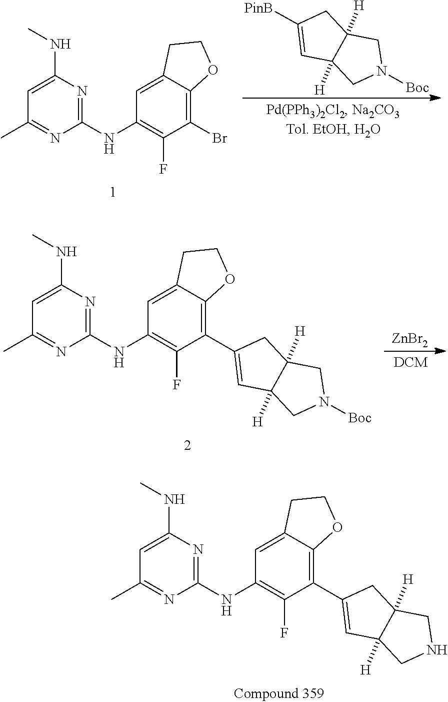

### Step 1. tert-butyl 5-[6-fluoro-5-[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate

To a solution of N2-(7-bromo-6-fluoro-2,3-dihydrobenzofuran-5-yl)-N4,6-dimethyl-pyrimidine-2,4-diamine (150 mg, 424.70 mol), tert-butyl 5-(4,4,5,5-tetramethyl-1,3,2-dioxaborolan-2-yl)-3,3a,6,6a-tetrahydro-1H-cyclopenta[c]pyrrole-2-carboxylate (170.85 mg, 509.64 mol), K2CO3 (176.09 mg, 1.27 mmol) in dioxane (5 mL) and H₂O (0.5 mL) was added cyclopentyl (diphenyl) phosphane;dichloropalladium;iron (31.08 mg, 42.47 μmol), then the mixture was stirred at 100° C. for 12 h under N₂ atmosphere of nitrogen. The reaction was concentrated to give tert-butyl 5-[6-fluoro-5-[[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate (204 mg, crude) as a yellow solid which was used for next step directly.

### Step 2. N2-[7-(1,2,3,3a,6,6a-hexahydrocyclopenta[c]pyrrol-5-yl)-6-fluoro-2,3-dihydrobenzofuran-5-yl]-N4,6-dimethyl-pyrimidine-2,4-diamine

To a solution of tert-butyl 5-[6-fluoro-5-[[4-methyl-6-(methylamino)pyrimidin-2-yl]amino]-2,3-dihydrobenzofuran-7-yl]-3,3a,6,6a-tetrahydro-1H-cyclopenta[c]pyrrole-2-carboxylate (204 mg, 423.62 μmol) in DCM (4 mL) was added TFA (2 mL), then the mixture was stirred at 25° C. for 1 h. The reaction was filtered and concentrated under reduced pressure to give a residue. The residue was purified by prep-HPLC (TFA condition, column: Phenomenex luna C18 100*40 mm*5 um; mobile phase: [water(0.1% TFA)-ACN]; B %: 1%-45%, 8 min) to give N2-[7-(1,2,3,3a,6,6a-hexahydrocyclopenta[c]pyrrol-5-yl)-6-fluoro-2,3-dihydrobenzofuran-5-yl]-N4,6-dimethyl-pyrimidine-2,4-diamine (4.3 mg, 11.27 μmol, 2.66% yield) (purity: 100%) as a white solid.

¹H NMR (400 MHz, METHANOL-d₄) δ: 2.15 (s, 3H) 2.77 (s, 4H) 3.01-3.14 (m, 5H) 3.27-3.34 (m, 1H) 3.34-3.40 (m, 2H) 3.649 (br., 1H) 4.52-4.59 (m, 2H) 5.82 (s, 1H) 6.13 (s, 1H) 7.46 (d, J=7.6 Hz, 1H), 8.38 (br., 1H)

### EXAMPLE 72. Synthesis of Compound 437

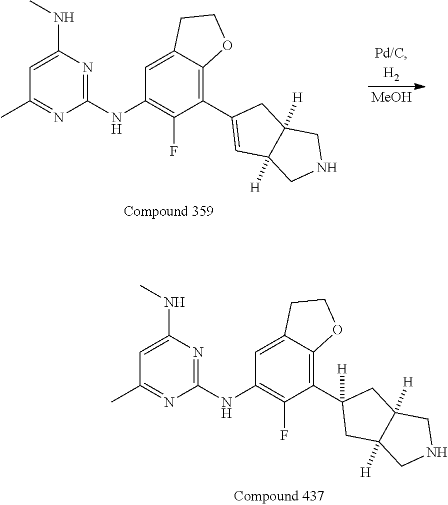 [SMILES: "MeOH" ; CNc1cc(C)nc(Nc2cc3c(c(C4=C[C@@H]5CNC[C@@H]5C4)c2F)OCC3)n1 ; CNc1cc(C)nc(Nc2cc3c(c([C@H]4C[C@H]5CNC[C@H]5C4)c2F)OCC3)n1]

### Step 1. N2-[7-(1, 2, 3, 3a, 4,5,6,6a-octahydrocyclopenta[c]pyrrol-5-yl)-6-fluoro-2,3-dihydrobenzofuran-5-yl]-N4,6-dimethyl-pyrimidine-2,4-diamine

To a solution of N2-[7-(1,2,3,3a,6,6a-hexahydrocyclopenta[c]pyrrol-5-yl)-6-fluoro-2,3-dihydrobenzofuran-5-yl]-N4,6-dimethyl-pyrimidine-2,4-diamine (20 mg, 52.43 μmol) in MeOH (1 mL), then the mixture was stirred at 25° C. for 1 h. The reaction was filtered and concentrated under reduced pressure to give a residue. The residue was purified by prep-HPLC (TFA condition, column: Phenomenex luna C18 100*40 mm*5 um; mobile phase: [water(0.1% TFA)-ACN]; B %: 1%-45%, 8 min) to give N2-[7-(1,2,3,3a,4,5,6,6a-octahydrocyclopenta[c]pyrrol-5-yl)-6-fluoro-2,3-dihydrobenzofuran-5-yl]-N4,6-dimethyl-pyrimidine-2,4-diamine (17.2 mg, 44.85 μmol, 85.55% yield) as a white solid.

¹H NMR (400 MHz, METHANOL-d₄) δ: 1.98 (td, J=12.16, 7.99 Hz, 2H) 2.16-2.25 (m, 2H) 2.25-2.33 (m, 3H) 2.85-2.91 (m, 3H) 2.93-3.02 (m, 2H) 3.16-3.28 (m, 1H) 3.41-3.59 (m, 3H) 4.60-4.68 (m, 2H) 4.93 (br s, 11H) 5.88-6.04 (m, 1H) 7.35-7.50 (m, 1H)

### EXAMPLE 73. Synthesis of Compound 363

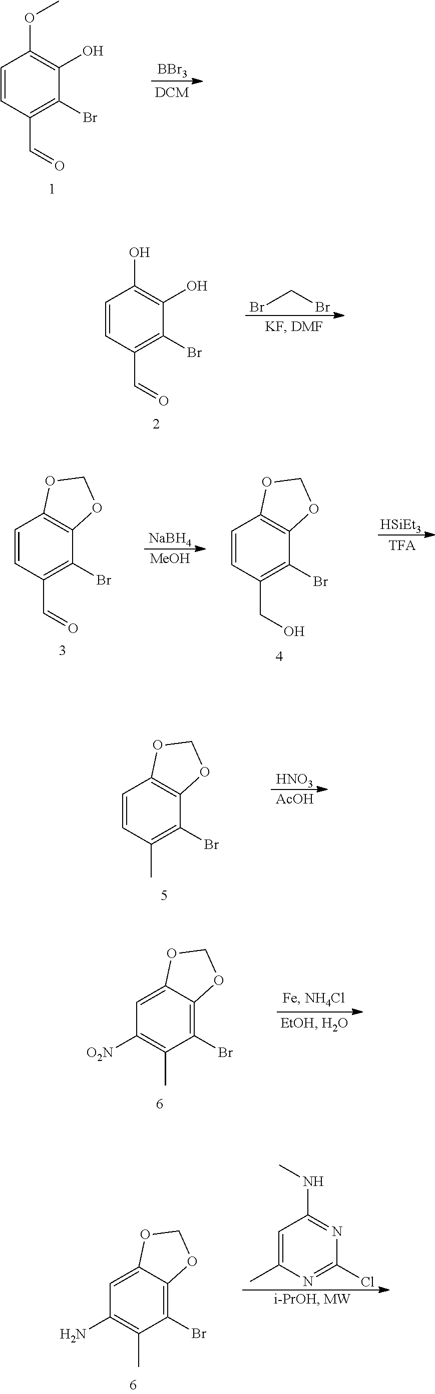

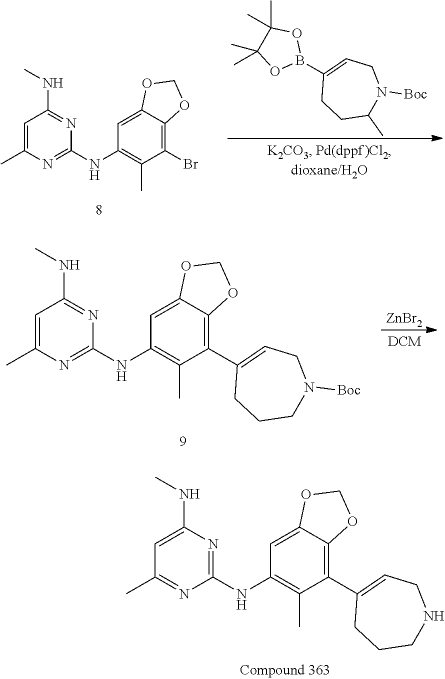 [SMILES: CNc1cc(C)nc(Nc2cc3c(c(Br)c2C)OCO3)n1 ; CC1CCC(B2OC(C)(C)C(C)(C)O2)=CCN1C(=O)OC(C)(C)C ; CNc1cc(C)nc(Nc2cc3c(c(C4=CCN(C(=O)OC(C)(C)C)CCC4)c2C)OCO3)n1 ; "ZnBr2" ; CNc1cc(C)nc(Nc2cc3c(c(C4=CCNCCC4)c2C)OCO3)n1]

### Step 1. 2-bromo-3,4-dihydroxy-benzaldehyde

To a solution of 2-bromo-3-hydroxy-4-methoxy-benzaldehyde (9 g, 12.98 mmol) in DCM (180 mL) was added BBr₃ (39.03 g, 51.94 mmol) at −78° C. under N2, then the mixture was stirred at 25° C. for 12 hrs under N2. The reaction mixture was quenched by MeOH (90 mL) at −78° C. under N2. The mixture was added to water (100 mL), extracted with EtOAc (100 mL*3) and dried over anhydrous sodium sulfate, filtered and concentrated under reduced pressure to give crude product. The residue was purified by column chromatography on silica gel to give 2-bromo-3,4-dihydroxy-benzaldehyde (8 g, crude) as a yellow solid.

### Step 2. 4-bromo-1,3-benzodioxole-5-carbaldehyde

To a mixture of 2-bromo-3,4-dihydroxy-benzaldehyde (4 g, 18.43 mmol) in DMF (320 mL) was added KF (5.35 g, 92.16 mmol) under N2 for 0.25 h, dibromomethane (3.52 g, 20.27 mmol) at 25° C. under N2, then the mixture was stirred at 115° C. for 4 hrs. The reaction mixture was added H₂O (300 mL) and extracted with EtOAc (200 mL*3) and dried over anhydrous sodium sulfate, filtered and concentrated under reduced pressure to give crude product. The residue was purified by column chromatography on silica gel. 4-bromo-1,3-benzodioxole-5-carbaldehyde (5 g, crude) was obtaiendas a yellow solid.

### Step 3. (4-bromo-1,3-benzodioxol-5-yl)methanol

To a solution of 4-bromo-1,3-benzodioxole-5-carbaldehyde (5 g, 21.83 mmol) in MeOH (100 mL) was added in portions NaBH₄ (1.24 g, 32.75 mmol) at 0° C., then the mixture was stirred at 25° C. for 1 h. The reaction was concentrated under reduced pressure, then added water (100 mL) and extracted with ethyl acetate (100 mL*3). The organic layer was dried over anhydrous sodium sulfate, filtered and concentrated under reduced pressure to give (4-bromo-1,3-benzodioxol-5-yl)methanol (5 g, crude) as a yellow solid.

### Step 4. 4-bromo-5-methyl-, 3-benzodioxole

To a solution of (4-bromo-1,3-benzodioxol-5-yl)methanol (1 g, 4.33 mmol) in DCM (50 mL) was added triethylsilane (1.01 g, 8.66 mmol, 1.38 mL) and TFA (3.95 g, 34.63 mmol, 2.67 mL), then the mixture was stirred at 25° C. under N2 for 2 hrs. The reaction was concentrated under reduced pressure, then added water (50 mL) and extracted with ethyl acetate (50 mL*3). The combined organic layers were washed with brine (50 mL*3) and dried over anhydrous sodium sulfate, filtered and concentrated under reduced pressure to give a residue. The residue was purified by column chromatography on silica gel to give 4-bromo-5-methyl-1,3-benzodioxole (200 mg, crude) as a white solid.

### Step 5. 4-bromo-5-methyl-6-nitro-1,3-benzodioxole

To a solution of 4-bromo-5-methyl-1,3-benzodioxole (200 mg, 930.04 mol) in TFA (8 mL) was added KNO₃ (103.43 mg, 1.02 mmol) at 0° C., then the mixture was stirred at 25° C. for 1 h. The reaction was added into water (30 mL) at 0° C., then extracted with ethyl acetate (30 mL*3). The combined organic layers were washed with saturated sodium bicarbonate solution (20 mL*3) and dried over anhydrous sodium sulfate, filtered and concentrated under reduced pressure to give 4-bromo-5-methyl-6-nitro-1,3-benzodioxole (400 mg, crude) as a yellow oil.

### Step 6. 7-bromo-6-methyl-1,3-benzodioxol-5-amine

To a solution of 4-bromo-5-methyl-6-nitro-1,3-benzodioxole (400 mg, 1.54 mmol) and NH₄Cl (822.81 mg, 15.38 mmol) in EtOH (25 mL) and H₂O (5 mL) was added Fe (429.51 mg, 7.69 mmol), and then the mixture was stirred at 80° C. for 4 hrs. The reaction was filtered and concentrated under reduced pressure, then added water (50 mL) extracted with ethyl acetate (50 mL*3). The organic layer was dried over anhydrous sodium sulfate, filtered and concentrated under reduced pressure to give 7-bromo-6-methyl-1,3-benzodioxol-5-amine (160 mg, crude) as a yellow solid.

### Step 7. N2-(7-bromo-6-methyl-1,3-benzodioxol-5-yl)-N4,6-dimethyl-pyrimidine-2,4-diamine

To a solution of 2-chloro-N,6-dimethyl-pyrimidin-4-amine (109.61 mg, 695.48 mol) and 7-bromo-6-methyl-1,3-benzodioxol-5-amine (160 mg, 695.48 μmol) in i-PrOH (1 mL) was added TFA (79.30 mg, 695.48 mol, 53.58 μL), then the mixture was stirred at 140° C. in the microwave for 2 hrs. The mixture was filtered and the filter cake was dried in vacuum to give N2-(7-bromo-6-methyl-1,3-benzodioxol-5-yl)-N4,6-dimethyl-pyrimidine-2,4-diamine (170 mg, crude) as a brown solid.

### Step 8. tert-butyl 2-methyl-5-[5-methyl-6-[[4-methyl-6-(methylamino)pyrimidin-2-yl]amino]-1,3-benzodioxol-4-yl]-2,3,4,7-tetrahydroazepine-1-carboxylate

To a mixture of tert-butyl 2-methyl-5-(4,4,5,5-tetramethyl-1,3,2-dioxaborolan-2-yl)-2,3,4,7-tetrahydroazepine-1-carboxylate (80 mg,237.21 μmol) and N2-(7-bromo-6-methyl-1,3-benzodioxol-5-yl)-N4,6-dimethyl-pyrimidine-2,4-diamine (83.31 mg, 237.21 mol) in H₂O (0.2 mL) and dioxane (2 mL) was added K₂CO₃ (65.57 mg, 474.41 mol) and cyclopentyl (diphenyl) phosphane;dichloromethane;dichloropalladium;iron (19.37 mg, 23.72 μmol) under N2 atmosphere. The reaction mixture was stirred at 100° C. for 12 hrs under N₂ atmosphere. The reaction was filtered and concentrated under reduced pressure to give tert-butyl 2-methyl-5-[5-methyl-6-[[4-methyl-6-(methylamino)pyrimidin-2-yl]amino]-1,3-benzodioxol-4-yl]-2,3,4,7-tetrahydroazepine-1-carboxylate (100 mg, crude) as brown oil which was used for next step directly without further purification.

### Step 9. N4,6-dimethyl-N2-[6-methyl-7-(2,3,4,7-tetrahydro-1H-azepin-5-yl)-1,3-benzodioxol-5-yl]pyrimidine-2,4-diamine

To a mixture of tert-butyl 5-[5-methyl-6-[[4-methyl-6-(methylamino)pyrimidin-2-yl]amino]-1,3-benzodioxol-4-yl]-2,3,4,7-tetrahydroazepine-1-carboxylate (70 mg, 149.71 μmol) in DCM (2 mL) was added ZnBr₂ (168.58 mg, 748.57 mol) under N₂ atmosphere. The reaction mixture was stirred at 30° C. for 3 hrs under N₂ atmosphere. The reaction was filtered and concentrated under reduced pressure to give a residue. The crude product was purified by prep-HPLC (TFA condition:column:Phenomenex Luna 80*30 mm*3 um; mobile phase:water (TFA)-ACN; B %:1%-30%,8 min. N4,6-dimethyl-N2-[6-methyl-7-(2,3,4,7-tetrahydro-1H-azepin-5-yl)-1,3-benzodioxol-5-yl]pyrimidine-2,4-diamine (36 mg, 97.97 μmol, 65.44% yield) was obtained as a pale yellow solid.

¹H NMR (400 MHz, CD₃OD, 298 K) δ: 6.87 (s, 1H), 5.98 (s, 2H), 5.94 (s, 1H), 5.85-5.79 (m, 1H), 3.93 (d, J=6.1 Hz, 2H), 3.53-3.46 (m, 2H), 2.88 (s, 3H), 2.72-2.65 (m, 2H), 2.25 (s, 3H), 2.14-2.07 (m, 5H)

### EXAMPLE 74. Synthesis of Compound 366 and Compound 366-a

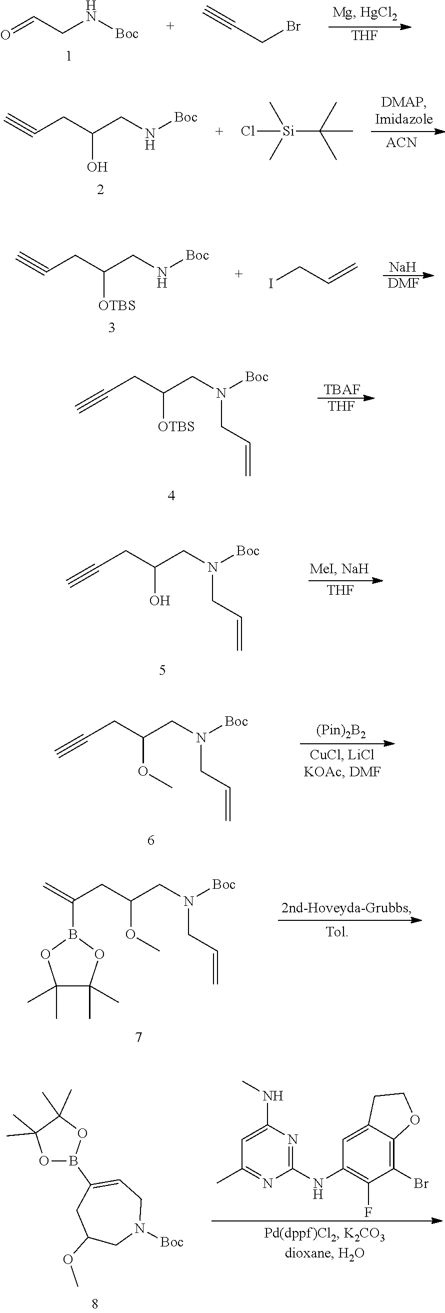

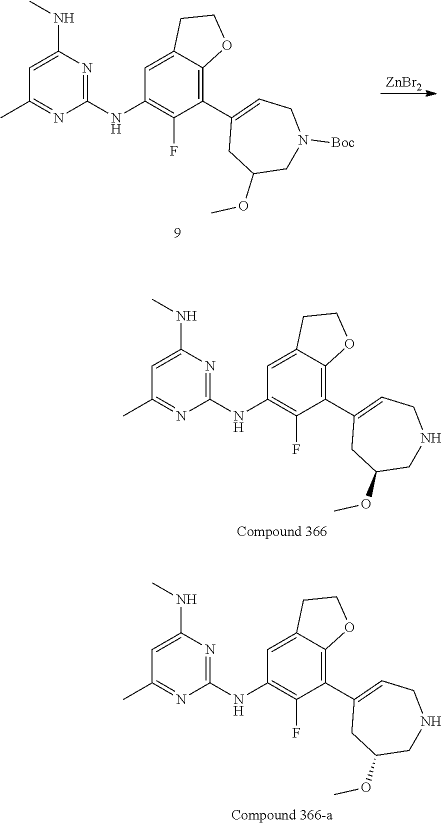 [SMILES: CNc1cc(C)nc(Nc2cc3c(c(C4=CCN(C(=O)OC(C)(C)C)CC(OC)C4)c2F)OCC3)n1 ; "ZnBr2" ; CNc1cc(C)nc(Nc2cc3c(c(C4=CCNC[C@@H](OC)C4)c2F)OCC3)n1 ; CNc1cc(C)nc(Nc2cc3c(c(C4=CCNC[C@H](OC)C4)c2F)OCC3)n1]

### Step 1. tert-butyl N-(2-hydroxypent-4-ynyl)carbamate

Mg (3.7 g, 152.23 mmol) were flame dried undervacuum, suspended in THF (55 mL), and treated with HgCl2 (150 mg, 552.49 μmol). The mixture was stirred at 25° C. for 30 min then cooled to 4° C., 3-bromoprop-1-yne (1.17 g, 7.85 mmol) was added to the above mixture. After addition, the mixture was stirred for 15 min at 25° C. and a rise in temperature was observed. The solution was maintained at 4° C. and the remainder of the 3-bromoprop-1-yne (13.45 g, 113.08 mmol) was added dropwise. The mixture was stirred at 0° C. for an additional 30 min, and then the mixture was transferred via cannula to a flask cooled to −42° C. tert-butyl N-(2-oxoethyl)carbamate (5 g, 31.41 mmol) in THF (10 mL) was added dropwise at −40° C. The reaction mixture was warmed to 25° C. and stirred at 25° C. for 12 h. The mixture was poured into a cold saturated NH₄Cl solution at 0° C., producing vigorous bubbling. The aqueous layer was extracted with EtOAc (30 mL*3). The organic layers were combined and dried over Na₂SO₄, filtered and concentrated in vacuum to give a residue which was purified by flash column chromatography on silica gel. tert-butyl N-(2-hydroxypent-4-ynyl)carbamate (5 g, 25.09 mmol, 79.89% yield) was obtained as a colourless oil.

### Step 2. tert-butyl N-[2-[tert-butyl(dimethyl)silyl]oxypent-4-ynyl]carbamate

The tert-butyl N-(2-hydroxypent-4-ynyl)carbamate (1.5 g, 7.53 mmol) was dissolved in MeCN (20 mL), tert-butyl-chloro-dimethyl-silane (1.70 g, 11.29 mmol, 2.10 mL), Imidazole (1.03 g, 15.06 mmol) and N,N-dimethyl]pyridin-4-amine (1.38 g, 11.29 mmol) were then added at 25° C. After addition, the reaction mixture was stirred at 25° C. for 12 h. The mixture was concentrated at 45° C., and dissolved in NaHCO₃ (50 mL), and then extracted with EtOAc (20 mL*2), and then the organic phase was concentrated in vacuum to give a residue which was purified by flash column chromatography on silica gel to give tert-butyl N-[2-[tert-butyl(dimethyl)silyl]oxypent-4-ynyl]carbamate (2.3 g, 7.34 mmol, 97.45% yield) as a colourless oil.

### Step 3. ethyl 4-(tert-butoxycarbonylamino)pentanoate

tert-butyl N-[2-[tert-butyl(dimethyl)silyl]oxypent-4-ynyl]carbamate (31 g, 98.88 mmol) was dissolved in DMF (310 mL), and then to the mixture was added NaH (4.75 g, 118.66 mmol, 60% purity) at 0° C., and then the mixture was stirred at 0° C. for 30 min, and then to the mixture was added 3-iodoprop-1-ene (19.93 g, 118.66 mmol, 10.85 mL) at 0° C., the mixture was stirred at 25° C. for 12 h. The reaction was quenched by sat. aq. NH₄Cl (300 mL), and then extracted with EtOAc (150 mL*2), and then the mixture was concentrated in vacuum to give a residue which was purified by prep-TLC to give tert-butyl N-allyl-N-[2-[tert-butyl(dimethyl)silyl]oxypent-4-ynyl]carbamate (31 g, crude) as a colourless oil.

¹H NMR (400 MHz, CHLOROFORM-d) δ: 0.02-0.13 (m, 6H) 0.85-0.94 (m, 9H) 1.39-1.52 (m, 9H) 1.95-2.05 (m, 1H) 2.29-2.43 (m, 2H) 2.92-3.06 (m, 1H) 3.43-3.55 (m, 1H)3.63-3.78 (m, 1H)3.94-4.19 (m, 2H)4.98-5.19 (m, 2H)5.65-5.85 (m, 1H)

### Step 4. tert-butyl N-allyl-N-(2-hydroxypent-4-ynyl)carbamate

Tert-butyl N-allyl-N-[2-[tert-butyl(dimethyl)silyl]oxypent-4-ynyl]carbamate (5 g, 14.14 mmol) was dissolved in THF (50 mL), and then the mixture was stirred at 25° C. for 2 h. The reaction mixture was concentrated to give a residue which was purified by flash column chromatography on silica gel to give tert-butyl N-allyl-N-(2-hydroxypent-4-ynyl)carbamate (3.2 g, crude) as colourless oil.

### Step 5. tert-butyl N-allyl-N-(2-methoxypent-4-ynyl)carbamate

tert-butyl N-allyl-N-(2-hydroxypent-4-ynyl)carbamate (3.2 g, 13.37 mmol) iodomethane (2.28 g, 16.05 mmol, 998.93 μL) was dissolved in THF (100 mL), and then to the mixture was added NaH (588.30 mg, 14.71 mmol, 60% purity) at 0° C., and then to the mixture was stirred at 25° C. for 1 h. The mixture was quenched by NH₄Cl(15 mL), and then extracted with EtOAc (20 mL*4), and then the organic phase was concentrated in vacuum to give a residue
which was purified by flash column chromatography on silica gel to give tert-butyl N-allyl-N-(2-methoxypent-4-ynyl)carbamate (1.6 g, crude) as colourless oil.

¹H NMR (400 MHz, CHLOROFORM-d) δ: 5.83-5.66 (m, 1H), 5.16-5.02 (m, 2H), 4.23-3.76 (m, 2H), 3.63-3.46 (m, 1H), 3.43-3.36 (m, 3H), 3.30-3.10 (m, 1H), 2.51-2.28 (m, 2H), 2.06-1.94 (m, 1H), 1.51-1.39 (m, 9H)

### Step 6. tert-butyl N-allyl-N-[2-methoxy-4-(4,4,5,5-tetramethyl-1,3,2-dioxaborolan-2-yl)pent-4-enyl]carbamate

To a solution of lithium;chloride (321.30 mg, 7.58 mmol) in DMF (30 mL) was added cuprous;chloride (750.30 mg, 7.58 mmol), then the mixture was stirred at 20° C. for 3 hrs under N2. potassium;acetate (743.81 mg, 7.58 mmol), 4,4,5,5-tetramethyl-2-(4,4,5,5-tetramethyl-1,3,2-dioxaborolan-2-yl)-1,3,2-dioxaborolane (1.92 g, 7.58 mmol) and tert-butyl N-allyl-N-(2-methoxypent-4-ynyl)carbamate (1.6 g, 6.32 mmol) was added to the mixture and then the reaction mixture was stirred at 25° C. for 12 hrs under N2. The reaction was quenched by NH₄Cl (300 mL), extracted with EtOAc (150 mL*3). The organic layer was dried over Na₂SO₄, concentrated to give the crude product which was purified by column chromatography on silica gel to give tert-butyl N-allyl-N-[2-methoxy-4-(4,4,5,5-tetramethyl-1,3,2-dioxaborolan-2-yl)pent-4-enyl]carbamate (1.1 g, crude) as a yellow oil.

### Step 7. tert-butyl 3-methoxy-5-(4,4,5,5-tetramethyl-1,3,2-dioxaborolan-2-yl)-2,3,4,7-tetrahydroazepine-1-carboxylate (130 mg, crude)

tert-butyl N-allyl-N-[2-methoxy-4-(4,4,5,5-tetramethyl-1,3,2-dioxaborolan-2-yl)pent-4-enyl]carbamate (500 mg, 1.31 mmol) was dissolved in DCM (50 mL), and then to the mixture was added Hoveyda-Grubbs Catalyst 2nd Generation (82.17 mg, 131.13 mol). The mixture was stirred at 25° C. for 12 h. The reaction was concentrated under reduced pressure to give a residue which was purified by column chromatography on silica gel to give tert-butyl 3-methoxy-5-(4,4,5,5-tetramethyl-1,3,2-dioxaborolan-2-yl)-2,3,4,7-tetrahydroazepine-1-carboxylate (130 mg, crude) as yellow oil.

### Step 8. tert-butyl 5-[6-fluoro-5-[[4-methyl-6-(methylamino)pyrimidin-2-yl]amino]-2,3-dihydrobenzofuran-7-yl]-3-methoxy-2,3,4,7-tetrahydroazepine-1-carboxylate

To a mixture of tert-butyl 3-methoxy-5-(4,4,5,5-tetramethyl-1,3,2-dioxaborolan-2-yl)-2,3,4,7-tetrahydroazepine-1-carboxylate (120 mg, 339.69 μmol), N2-(7-bromo-6-fluoro-2,3-dihydrobenzofuran-5-yl)-N4,6-dimethyl-pyrimidine-2,4-diamine (119.98 mg, 339.69 μmol) in dioxane (5 mL) and H₂O (0.5 mL) was added K₂CO₃ (93.89 mg, 679.39 mol) and cyclopentyl(diphenyl)phosphane;dichloromethane;dichloropalladium;iron (27.74 mg, 33.97 mol) under N₂ atmosphere. The reaction mixture was stirred at 100° C. for 12 h under N2.

The reaction was concentrated under reduced pressure to give tert-butyl 5-[6-fluoro-5-[[4-methyl-6-(methylamino)pyrimidin-2-yl]amino]-2,3-dihydrobenzofuran-7-yl]-3-methoxy-2,3,4,7-tetrahydroazepine-1-carboxylate (150 mg, crude) as yellow solid.

### Step 9. N2-[6-fluoro-7-[rel-(3S)-3-methoxy-2,3,4,7-tetrahydro-1H-azepin-5-yl]-2,3-dihydrobenzofuran-5-yl]-N4,6-dimethyl-pyrimidine-2,4-diamine and N2-[6-fluoro-7-[rel-(3R)-3-methoxy-2,3,4,7-tetrahydro-1H-azepin-5-yl]-2,3-dihydrobenzofuran-5-yl]-N4,6-dimethyl-pyrimidine-2,4-diamine

To a solution of tert-butyl 5-[6-fluoro-5-[[4-methyl-6-(methylamino)pyrimidin-2-yl]amino]-2,3-dihydrobenzofuran-7-yl]-3-methoxy-2,3,4,7-tetrahydroazepine-1-carboxylate (130 mg, 260.22 μmol) in DCM (5 mL) was added ZnBr₂ (293.01 mg, 1.30 mmol). The reaction mixture was stirred at 35° C. for 2 h under N2. The reaction was concentrated under reduced pressure to give the residue. The crude product was purified by prep-HPLC(TFA condition:column:3_Phenomenex Luna C18 75*30 mm*3 um; mobile phase:water(0.2% TFA)-ACN; B %: 1%-30%,8 min). The residue was separated by Chiral SFC (Column: DAICEL CHIRALPAK 1H(250 mm*30 mm,10 um); Mobile phase: A for Heptane and B for IPA(NH₃·H₂O); SFC, Acq Method: IH10_H_I_Ib_30_10_MS) to give N2-[6-fluoro-7-[rel-(3S)-3-methoxy-2,3,4,7-tetrahydro-1H-azepin-5-yl]-2,3-dihydrobenzofuran-5-yl]-N4,6-dimethyl-pyrimidine-2,4-diamine (2.9 mg, 7.26 μmol, 2.79% yield, Rt=2.805 min) was obtained as a yellow solid.

N2-[6-fluoro-7-[rel-(3R)-3-methoxy-2,3,4,7-tetrahydro-1H-azepin-5-yl]-2,3-dihydrobenzofuran-5-yl]-N4,6-dimethyl-pyrimidine-2,4-diamine (3.8 mg, 9.51 μmol, 3.66% yield, (Rt=3.528 min)) was obtained as yellow solid.

¹H NMR (400 MHz, METHANOL-d₄) δ: 7.90-7.79 (m, 1H), 6.07 (t, J=5.7 Hz, 1H), 5.84-5.75 (m, 1H), 4.58 (dt, J=5.0, 8.7 Hz, 2H), 3.62-3.55 (m, 1H), 3.54-3.49 (m, 2H), 3.40-3.33 (m, 4H), 3.20 (br t, J=8.4 Hz, 2H), 2.98 (dd, J=7.8, 13.4 Hz, 1H), 2.86 (s, 3H), 2.83-2.78 (m, 2H), 2.16 (s, 3H)

¹H NMR (400 MHz, METHANOL-d₄) δ: 7.89-7.79 (m, 1H), 6.10-6.03 (m, 1H), 5.83-5.76 (m, 1H), 4.58 (dt, J=4.8, 8.7 Hz, 2H), 3.59-3.53 (m, 1H), 3.52-3.47 (m, 2H), 3.39-3.35 (m, 4H), 3.20 (br t, J=8.3 Hz, 2H), 2.95 (dd, J=7.9, 13.3 Hz, 1H), 2.86 (s, 3H), 2.82-2.77 (m, 2H), 2.16 (s, 3H)

### EXAMPLE 75. Synthesis of Compound 368-a and Compound 368

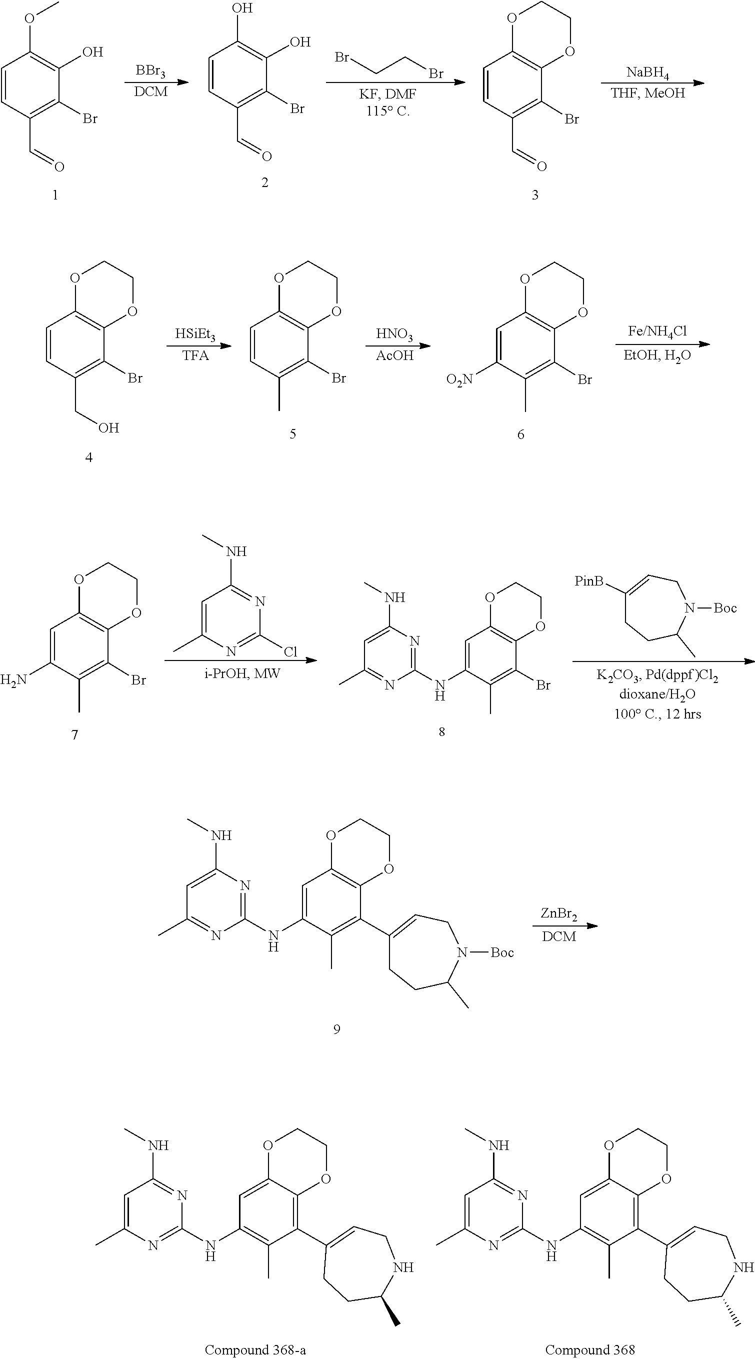

### Step 1. 2-bromo-3,4-dihydroxy-benzaldehyde

To a solution of 2-bromo-3-hydroxy-4-methoxy-benzaldehyde (9 g, 12.98 mmol) in DCM (500 mL) was added BBr₃ (39.03 g, 51.94 mmol) at −78° C. under N2, then the mixture was stirred at 25° C. for 12 h under N2. The reaction mixture was quenched by MeOH (300 mL) at −78° C. under N2 and the reaction mixture was added water (500 mL), extracted with EtOAc (500 mL*3) and dried over anhydrous sodium sulfate, filtered and concentrated under reduced pressure to give crude product. The residue was purified by column chromatography on silica gel to give 2-bromo-3,4-dihydroxy-benzaldehyde (8 g, crude) as a yellow solid.

### Step 2. 5-bromo-2,3-dihydro-1,4-benzodioxine-6-carbaldehyde

To a mixture of 2-bromo-3,4-dihydroxy-benzaldehyde (8 g, 18.43 mmol) in DMF (400 mL) was added KF (10.7 g, 92.16 mmol),1,2-dibromoethane (7.62 g, 20.27 mmol, 1.75 mL) at 25° C. under N2, then the mixture was stirred at 115° C. for 4 hrs under N2. The reaction mixture was added H₂O (200 mL) and extracted with EtOAc (200 mL*3) and dried over anhydrous sodium sulfate, filtered and concentrated under reduced pressure to give crude product. The residue was purified by column chromatography on silica gel to give 5-bromo-2,3-dihydro-1,4-benzodioxine-6-carbaldehyde (7.3 g, crude) as a yellow solid.

### Step 3. (5-bromo-2,3-dihydro-1,4-benzodioxin-6-yl)methanol

To a solution of 5-bromo-2,3-dihydro-1,4-benzodioxine-6-carbaldehyde (7.3 g, 30.03 mmol) in MeOH (100 mL) was added in portions NaBH₄ (1.70 g, 45.05 mmol) at 0° C., then the mixture was stirred at 25° C. for 1 h. The reaction was concentrated under reduced pressure, then added to water (100 mL) and extracted with ethyl acetate (100 mL*3). The organic layer was dried over anhydrous sodium sulfate, filtered and concentrated under reduced pressure to give (5-bromo-2,3-dihydro-1,4-benzodioxin-6-yl)methanol (7 g, crude) as a white solid.

### Step 4. 5-bromo-6-methyl-2,3-dihydro-1,4-benzodioxine

To a solution of (5-bromo-2,3-dihydro-1,4-benzodioxin-6-yl)methanol (2 g, 8.16 mmol) in DCM (100 mL) was added triethylsilane (1.90 g, 16.32 mmol, 2.61 mL) and TFA (7.44 g, 65.29 mmol, 5.03 mL), then the mixture was stirred at 25° C. under N2 for 2 hrs. The reaction was concentrated under reduced pressure, then added water (50 mL) and extracted with ethyl acetate (50 mL*3). The combined organic layers were washed with brine (50 mL*3) and dried over anhydrous sodium sulfate, filtered and concentrated under reduced pressure to give a residue. The residue was purified by column chromatography on silica gel to give 5-bromo-6-methyl-2,3-dihydro-1,4-benzodioxine (1 g, crude) as a white solid.

¹H NMR (400 MHz, CDCl₃, 298 K) δ: 6.75 (s, 2H), 4.41-4.34 (m, 2H), 4.26-4.22 (m, 2H), 2.35 (s, 3H)

### Step 5. 5-bromo-6-methyl-7-nitro-2,3-dihydro-1,4-benzodioxine

To a solution of 5-bromo-6-methyl-2,3-dihydro-1,4-benzodioxine (200 mg, 873.09 μmol) in AcOH (2 mL) was added a mixture of HNO₃ (550.16 mg, 8.73 mmol, 392.97 μL) at −5° C., then the mixture was stirred at 25° C. for 1 h. The reaction mixture was added to ice water (5 mL), extracted with EtOAc (20 mL*3). The combined organic layers were washed with 50 mL of brine, dried over Na₂SO₄, filtered and concentrated under reduced pressure to give 5-bromo-6-methyl-7-nitro-2,3-dihydro-1,4-benzodioxine (220 mg, 802.72 μmol) as a yellow solid.

¹H NMR (400 MHz, CDCl₃, 298 K) δ: 7.51 (s, 1H), 4.47-4.43 (m, 2H), 4.32-4.29 (m, 2H), 2.59 (s, 3H)

### Step 6. 5-bromo-6-methyl-2,3-dihydro-1,4-benzodioxin-7-amine

To a solution of 5-bromo-6-methyl-7-nitro-2,3-dihydro-1,4-benzodioxine (220 mg, 802.72 μmol) and NH₄Cl (429.39 mg, 8.03 mmol) in H₂O (5 mL) and EtOH (25 mL) was added Fe (224.14 mg, 4.01 mmol) and then the mixture was stirred at 80° C. for 4 hrs. The reaction was filtered and concentrated under reduced pressure, then added water (10 mL) and extracted with ethyl acetate (10 mL*3). The organic layer was dried over anhydrous sodium sulfate, filtered and concentrated under reduced pressure to give 5-bromo-6-methyl-2,3-dihydro-1,4-benzodioxin-7-amine (180 mg, crude) as a yellow solid.

### Step 7. N2-(5-bromo-6-methyl-2,3-dihydro-1,4-benzodioxin-7-yl)-N4,6-dimethyl-pyrimidine-2,4-diamine

To a solution of 2-chloro-N,6-dimethyl-pyrimidin-4-amine (109.77 mg, 696.48 mol) and 5-bromo-6-methyl-2,3-dihydro-1,4-benzodioxin-7-amine (170 mg, 696.48 μmol) in i-PrOH (2 mL) was added TFA (79.41 mg, 696.48 mol, 53.30 μL), then the mixture was stirred at 140° C. in the microwave for 2 hrs. The mixture was filtered and the filter cake was dried in vacuum to give N2-(5-bromo-6-methyl-2,3-dihydro-1,4-benzodioxin-7-yl)-N4,6-dimethyl-pyrimidine-2,4-diamine (240 mg, crude) as a brown solid.

### Step 8. tert-butyl 5-[6-methyl-7-[[4-methyl-6-(methylamino)pyrimidin-2-yl]amino]-2,3-dihydro-1,4-benzodioxin-5-yl]-2,3,4,7-tetrahydroazepine-1-carboxylate

To a mixture of N2-(5-bromo-6-methyl-2,3-dihydro-1,4-benzodioxin-7-yl)-N4,6-dimethyl-pyrimidine-2,4-diamine (30 mg, 82.14 μmol), tert-butyl 5-(4,4,5,5-tetramethyl-1,3,2-dioxaborolan-2-yl)-2,3,4,7-tetrahydroazepine-1-carboxylate (29.21 mg, 90.36 μmol) in H₂O (0.1 ml) and dioxane (1 ml) was added cyclopentyl (diphenyl)phosphane:dichloromethane;dichloropalladium;iron (6.71 mg, 8.21 μmol) and K₂CO₃ (22.70 mg, 164.28 mol) under N₂ atmosphere. The reaction mixture was stirred at 100° C. for 12 hrs under N₂ atmosphere. The reaction mixture was filtered and concentrated under reduced pressure to give tert-butyl 5-[6-methyl-7-[[4-methyl-6-(methylamino)pyrimidin-2-yl]amino]-2,3-dihydro-1,4-benzodioxin-5-yl]-2,3,4,7-tetrahydroazepine-1-carboxylate (30 mg, crude) as a brown solid.

### Step 9. N4,6-dimethyl-N2-[6-methyl-5-[rel-(2S)-2-methyl-2,3,4,7-tetrahydro-1H-azepin-5-yl-2,3-dihydro-1,4-benzodioxin-7-yl pyrimidine-2,4-diamine and N4,6-dimethyl-N2-[6-methyl-5-[rel-(2R)-2-methyl-2,3,4,7-tetrahydro-1H-azepin-5-yl]-2,3-dihydro-1,4-benzodioxin-7-yl]pyrimidine-2,4-diamine

To a mixture of tert-butyl 2-methyl-5-[6-methyl-7-[[4-methyl-6-(methylamino)pyrimidin-2-yl]amino]-2,3-dihydro-1,4-benzodioxin-5-yl]-2,3,4,7-tetrahydroazepine-1-carboxylate (100 mg, 201.77 μmol) in DCM (2 mL) was added ZnBr₂ (454.38 mg, 2.02 mmol) under N₂ atmosphere. The reaction mixture was stirred at 30° C. for 3 hrs under N₂ atmosphere. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give crude product. The crude product was purified by SFC (basic condition, Column: DAICEL CHIRALPAK AD(250 mm*30 mm,10 um); Mobile phase: A for Heptane and B for EtOH (NH₃·H₂O); Gradient: B %=30-30%) to give N4,6-dimethyl-N2-[6-methyl-5-[rel-(2S)-2-methyl-2,3,4,7-tetrahydro-1H-azepin-5-yl]-2,3-dihydro-1,4-benzodioxin-7-yl]pyrimidine-2,4-diamine (10.1 mg, 25.54 μmol, 12.66% yield) as a pale yellow solid and N4,6-dimethyl-N2-[6-methyl-5-[rel-(2R)-2-methyl-2,3,4,7-tetrahydro-1H-azepin-5-yl]-2,3-dihydro-1,4-benzodioxin-7-yl]pyrimidine-2,4-diamine (8.8 mg, 22.25 μmol, 11.03% yield) as a pale yellow solid.

¹H NMR (400 MHz, CD₃OD, 299 K) δ: 7.17 (br s, 1H), 5.75 (s, 1H), 5.67-5.55 (m, 1H), 4.19 (d, J=8.4 Hz, 4H), 3.55-3.38 (m, 2H), 3.06 (dtd, J=3.3, 6.2, 9.2 Hz, 1H), 2.83 (s, 3H), 2.68-2.44 (m, 1H), 2.37-2.24 (m, 1H), 2.21-2.04 (m, 6H), 2.00-1.84 (m, 1H), 1.80-1.64 (m, 1H), 1.18 (d, J=6.5 Hz, 3H)

¹H NMR (400 MHz, CD₃OD, 299 K) δ: 7.27-7.08 (m, 1H), 5.75 (s, 1H), 5.68-5.53 (m, 1H), 4.19 (br d, J=8.4 Hz, 4H), 3.58-3.37 (m, 2H), 3.14-3.00 (m, 1H), 2.83 (s, 3H), 2.67-2.45 (m, 1H), 2.38-2.25 (m, 1H), 2.21-2.05 (m, 6H), 2.00-1.86 (m, 1H), 1.81-1.66 (m, 1H), 1.19 (d, J=6.5 Hz, 3H)

### EXAMPLE 76. Synthesis of Compound 380 and Compound 369

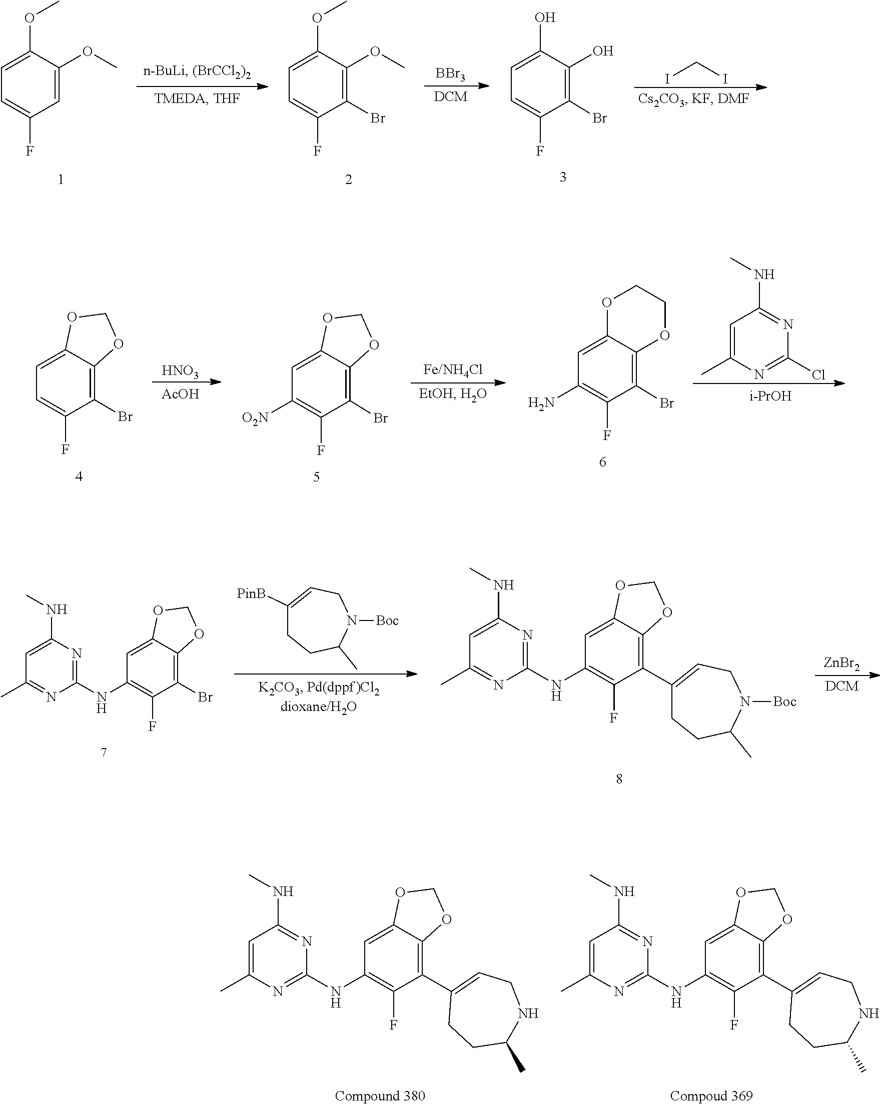

### Step 1. 2-bromo-1-fluoro-3,4-dimethoxy-benzene

n-butyllithium (2.5 M, 38.42 mL) was added dropwise at −78° C. to a solution of compound 4-fluoro-1,2-dimethoxy-benzene (10 g, 64.04 mmol, 8.54 mL) and N,N,N′,N′-tetramethylethane-1,2-diamine (7.44 g, 64.04 mmol, 9.60 mL) in THF (128 mL) under nitrogen atmosphere and stirred at −78° C. for 1.5 h. Then 1,2-dibromo-1,1,2,2-tetrachloro-ethane (27.32 g, 83.89 mmol, 10.07 mL) in THF (32 mL) was added, after stirring for a further 10 min, the cooling bath was removed and the reaction vessel allowed to warm to room temperature. The reaction mixture was quenched by sat. NH₄Cl (100 mL), extracted with EtOAc (300 mL*3).The organic layer was dried over Na₂SO₄, concentrated to give the crude product. The crude product was purified by column chromatography on silica gel to give 2-bromo-1-fluoro-3,4-dimethoxy-benzene (15 g, crude) as a yellow gum.

### Step 2. 3-bromo-4-fluoro-benzene-1,2-diol

To a mixture of 2-bromo-1-fluoro-3,4-dimethoxy-benzene (14.5 g, 61.69 mmol) in DCM (160 mL) was added BBr₃ (77.27 g, 308.45 mmol) in DCM (160 mL) at 0° C., then the mixture was stirred at 25° C. for 3 hrs. The reaction mixture was quenched by MeOH (150 mL) at 0° C. The reaction mixture was filtered and concentrated to give 3-bromo-4-fluoro-benzene-1,2-diol (10 g, crude) as a brown gum.

### Step 3. 4-bromo-5-fluoro-1,3-benzodioxole

To a mixture of 3-bromo-4-fluoro-benzene-1,2-diol (4.5 g, 21.74 mmol) in DMF (60 mL) was added C_{S2}CO₃ (21.25 g, 65.22 mmol), KF (1.26 g, 21.74 mmol), diiodomethane (11.65 g, 43.48 mmol, 3.50 mL) at 25° C., then the mixture was stirred at 100° C. for 12 hrs. The reaction mixture was added to water (150 mL), extracted with EtOAc (500 mL*3). The combined organic layers were washed with 500 mL of brine, dried over Na₂SO₄, filtered and concentrated under reduced pressure to give a residue. The crude product was purified by column chromatography on silica gel to give 4-bromo-5-fluoro-1,3-benzodioxole (3.3 g, crude) as a white solid.

### Step 4. 4-bromo-5-fluoro-6-nitro-1,3-benzodioxole

To a mixture of HNO₃ (2.09 g, 22.60 mmol, 68% purity) in AcOH (33 mL) was added 4-bromo-5-fluoro-1,3-benzodioxole (3.3 g, 15.07 mmol) at 0° C., then the mixture was stirred at 25° C. for 12 hrs. This reaction was set four batch in parallel. The reaction mixture was added to ice water (100 mL), the mixture was filtered and the filter cake was dried in vacuum to give 4-bromo-5-fluoro-6-nitro-1,3-benzodioxole (2.34 g, crude) as a yellow solid.

### Step 5. 7-bromo-6-fluoro-1,3-benzodioxol-5-amine

To a mixture of 7-bromo-6-methyl-5-nitro-2,3-dihydrobenzofuran (1 g, 3.87 mmol) in EtOH (20 mL), H₂O (2 mL) was added Fe (1.08 g, 19.37 mmol), NH₄Cl (2.07 g, 38.75 mmol) at 25° C., then the mixture was stirred at 80° C. for 12 hrs. The reaction mixture was filtered and concentrated to give the crude product. The crude product was added to water (50 mL), extracted with EtOAc (50 mL*3). The combined organic layers were washed with 10 mL of brine, dried over anhydrous Na₂SO₄, filtered and concentrated under reduced pressure to give the crude product. The crude product was purified by prep-TLC to give 7-bromo-6-fluoro-1,3-benzodioxol-5-amine (0.9 g, crude) as a yellow solid.

### Step 6. N2-(7-bromo-6-fluoro-1,3-benzodioxol-5-yl)-N4,6-dimethyl-pyrimidine-2,4-diamine

To a solution of 7-bromo-6-fluoro-1,3-benzodioxol-5-amine (310 mg, 1.32 mmol) and 2-chloro-N,6-dimethyl-pyrimidin-4-amine (208.77 mg, 1.32 mmol) in i-PrOH (6 mL) was added TFA (10 mg, 87.70 mol, 6.76 L), then the mixture was stirred at 140° C. for 2 hrs. The reaction was concentrated under reduced pressure to give N2-(7-bromo-6-fluoro-1,3-benzodioxol-5-yl)-N4,6-dimethyl-pyrimidine-2,4-diamine (240 mg, crude) as brown soild.

### Step 7. tert-butyl 2-methyl-5-[6-methyl-5-[[4-methyl-6-(methylamino)pyrimidin-2-ylamino-2,3-dihydrobenzo furan-7-yl]-2,3,4,7-tetrahydroazepine-1-carboxylate

To a mixture of N2-(7-bromo-6-fluoro-1,3-benzodioxol-5-yl)-N4,6-dimethyl-pyrimidine-2,4-diamine (80 mg, 225.25 μmol), terttert-butyl 2-methyl-5-(4,4,5,5-tetramethyl-1,3,2-dioxaborolan-2-yl) 2,3,4,7tetrahydroazepinelcarboxylate (75.97 mg, 225.25 mol) in dioxane (2 mL) and H₂O (0.2 mL) was added K₂CO₃ (62.26 mg, 450.50 mol) and cyclopentyl (diphenyl) phosphane;dichloromethane;dichloropalladium;iron (18.39 mg, 22.52 mol) under N2 atmosphere. Then the reaction mixture was stirred at 100° C. for 12 h under N₂ atmosphere. The reaction was concentrated under reduced pressure to give the mixture to give tert-butyl 2-methyl-5-[6-methyl-5-[[4-methyl-6-(methylamino)pyrimidin-2-yl]amino]2,3dihydrobenzofuran-7-yl]-2,3,4,7-tetrahydroazepine-1-carboxylate (108 mg, crude) yellow solid.

### Step 8. N2-[6-fluoro-7-[rel-(2S)-2-methyl-2,3,4,7-tetrahydro-1H-azepin-5-yl]-], 3-benzodioxol-5-yl]-N4,6-dimethyl-pyrimidine-2,4-diamine and N2-[6-fluoro-7-[rel-(2R)-2-methyl-2,3,4,7-tetrahydro-1H-azepin-5-yl]-1,3-benzodioxol-5-yl]-N4,6-dimethyl-pyrimidine-2,4-diamine

To a mixture of tert-butyl 5-[5-fluoro-6-[[4-methyl-6-(methylamino)pyrimidin-2-yl]amino]-1,3-benzodioxol-4-yl]-2-methyl-2,3,4,7-tetrahydroazepine-1-carboxylate (108 mg, 222.43 mol) in DCM (3 mL) was added ZnBr₂ (500.90 mg, 2.22 mmol) at 25° C., then the mixture was stirred at 25° C. for 12 hrs. The reaction mixture was filtered and purified by prep-HPLC; (TFA condition:column:Phenomenex Luna 80*30 mm*3 um; mobile phase:water (TFA)-ACN; B %: 1%-30%,8 min) to give the mixture of these two single isomer. The mixture was separated by SFC (column: DAICEL CHIRALPAKIC (250 mm*30 mm,10 um); mobile phase:[0.1% NH₃H₂O MEOH]; B %: 50%-55%; SFC, Acq Method: IC_MeOH_IPAm_5_50_3_35_3 min) to give N2-[6-fluoro-7-[rel-(2S)-2-methyl-2,3,4,7-tetrahydro-1H-azepin-5-yl]-1,3-benzodioxol-5-yl]-N4,6-dimethyl-pyrimidine-2,4-diamine (13.6 mg, 35.28 mol, 15.86% yield), Rt=2.215 min) as a yellow solid and N2-[6-fluoro-7-[rel-(2R)-2-methyl-2,3,4,7-tetrahydro-1H-azepin-5-yl]-1,3-benzodioxol-5-yl]-N4,6-dimethyl-pyrimidine-2,4-diamine (14 mg, 12.94 mol, 6.43% yield, Rt=2.387 min) as a yellow solid.

¹H NMR (400 MHz, METHANOL-d₄) δ:1.12-1.22 (m, 3H) 1.57-1.74 (m, 1H) 1.87-1.99 (m, 1H) 2.06-2.23 (m, 7H) 2.28-2.41 (m, 1H) 2.49-2.64 (m, 1H) 2.75-2.84 (m, 3H) 3.03-3.10 (m, 1H) 3.13-3.21 (m, 2H) 3.35-3.60 (m, 2H) 4.43-4.55 (m, 2H) 4.86 (s, 27H) 5.63-5.69 (m, 1H) 5.71-5.75 (m, 1H) 7.14-7.30 (m, 1H)

¹H NMR (400 MHz, METHANOL-d₄) δ: 1.14-1.23 (m, 3H) 1.51-1.97 (m, 2H) 2.17 (s, 3H) 2.54-2.73 (m, 2H) 2.88 (s, 3H) 3.03-3.15 (m, 1H) 3.43-3.57 (m, 2H) 5.79-5.84 (m, 1H) 5.91 (br d, J=4.52 Hz, 2H) 6.04-6.15 (m, 1H) 7.76-7.83 (m, 1H)

### EXAMPLE 77. Synthesis of Compound 371-a and Compound 371

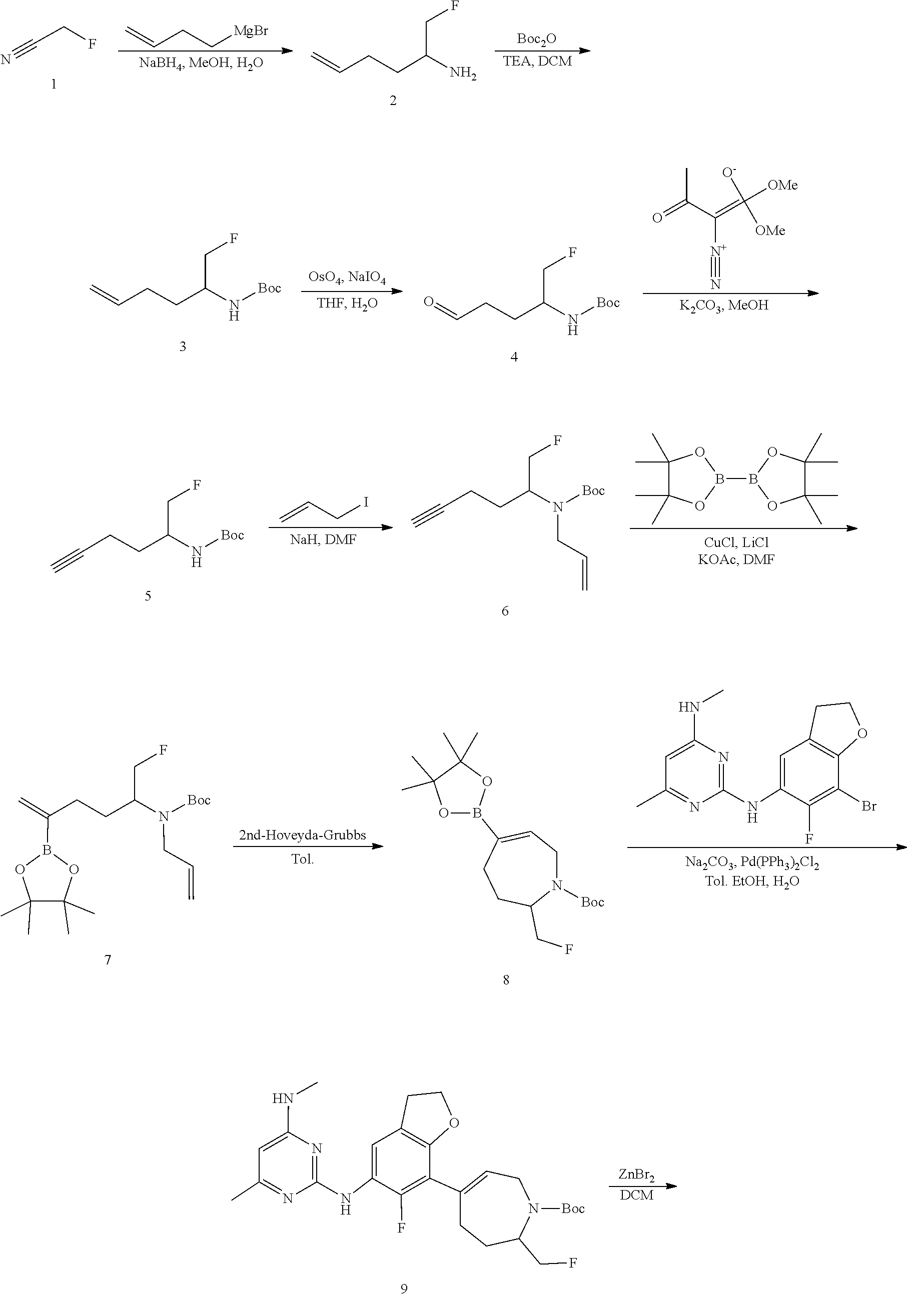

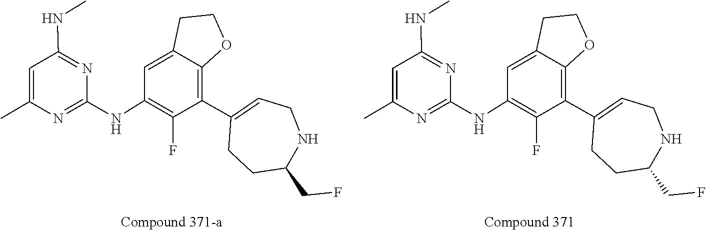 [SMILES: CNc1cc(C)nc(Nc2cc3c(c(C4=CCN[C@@H](CF)CC4)c2F)OCC3)n1 ; CNc1cc(C)nc(Nc2cc3c(c(C4=CCN[C@H](CF)CC4)c2F)OCC3)n1]

### Step 1. fluorohex-5-en-2-amine

2-fluoroacetonitrile (5 g, 84.69 mmol) was dissolved in THF (100 mL), and then to the mixture was added bromo(but-3-enyl)magnesium (0.5 M, 186.31 mL) at −20° C., and then the mixture was stirred at −20° C. for 1 h. NaBH₄ (3.52 g, 93.15 mmol) was added in H₂O (5 mL) MeOH (200 mL) in portions at −40° C., and then to the mixture was added dropwise the previous THF solution at −40° C., and then the mixture was stirred at 0° C. for 1 h. The mixture was quenched by H₂O (100 mL) and saturated ammonium chloride solution (100 mL), and then extracted with EtOAc (100 mL*2), and then the organic phase was concentrated in vacuum to give fluorohex-5-en-2-amine (3 g, crude) as a black oil.

### Step 2. tert-butyl N-[1-(fluoromethyl)pent-4-enyl]carbamate

1-fluorohex-5-en-2-amine (3 g, 25.61 mmol) was dissolved in DCM (60 mL), and then to the mixture was added Boc₂O (8.38 g, 38.41 mmol, 8.81 mL) TEA (5.18 g, 51.21 mmol, 7.14 mL), and then the mixture was stirred at 25° C. for 12 h. The mixture was concentrated in vacuo to give a residue. The residue was purified by flash chromatography on silica gel to give tert-butyl N-[1-(fluoromethyl)pent-4-enyl]carbamate (4.6 g, 21.17 mmol, 82.68% yield) as yellow oil.

### Step 3. tert-butyl N-[1]-(fluoromethyl)-4-oxo-butyl]carbamate

To a solution of tert-butyl N-[1-(fluoromethyl)pent-4-enyl]carbamate (4.66 g, 21.45 mmol) in THF (250 mL) and H₂O (50 mL) was added K₂OsO₄·2H₂O (790.23 mg, 2.14 mmol) and NaIO₄ (18.35 g, 85.79 mmol) at 0° C., then the mixture was stirred at 20° C. for 12 h. The reaction was added into water (300 mL), then extracted with ethyl acetate (500 mL*3). The combined organic layers were washed with brine (500 mL) and dried over anhydrous sodium sulfate, filtered and concentrated under reduced pressure to give tert-butyl N-[1-(fluoromethyl)-4-oxo-butyl]carbamate (4.7 g, crude) as a black oil.

### Step 4. tert-butyl N-[1]-(fluoromethyl)pent-4-ynyl]carbamate

To a solution of tert-butyl N-[1-(fluoromethyl)-4-oxo-butyl]carbamate (4.7 g, 21.44 mmol) and (Z)-1-diazonio-1-dimethoxyphosphoryl-prop-1-en-2-olate (4.12 g, 21.44 mmol) in MeOH (100 mL) was added K₂CO₃ (5.93 g, 42.87 mmol) at 0° C., then the mixture was stirred at 20° C. for 12 h. The reaction was concentrated under reduced pressure, then added into water (100 mL), then extracted with ethyl acetate (100 mL*3). The combined organic layers were washed with brine (200 mL*3) and dried over anhydrous sodium sulfate, filtered and concentrated under reduced pressure to give a residue. The residue was purified by flash chromatography on silica gel to give tert-butyl N-[1-(fluoromethyl)pent-4-ynyl]carbamate (1.2 g, crude) as a light-yellow oil.

¹H NMR (400 MHz, CHLOROFORM-d) δ: 1.49 (s, 9H) 1.77-1.86 (m, 2H) 1.99 (br t, J=2.63 Hz, 1H) 2.31 (td, J=7.25, 2.63 Hz, 2H) 3.83-3.97 (m, 1H) 4.39 (br s, 1H) 4.51 (br d, J=2.63 Hz, 1H)

### Step 5. tert-butyl N-allyl-N-1-(fluoromethyl)pent-4-ynyl]carbamate

To a solution of tert-butyl N-[1-(fluoromethyl)pent-4-ynyl]carbamate (1 g, 4.65 mmol) in DMF (20 mL) was added NaH (213.67 mg, 5.34 mmol, 60% purity) at 0° C., then the mixture was stirred at 0° C. for 0.5 h. Then the mixture was added a solution of 3-iodoprop-1-ene (858.36 mg, 5.11 mmol, 467.26 μL), then the mixture was stirred at 20° C. for 2 h. The reaction was added into saturated ammonium chloride solution (50 mL), then extracted with ethyl acetate (50 mL*3). The combined organic layers were washed with brine (100 mL*3) and dried over anhydrous sodium sulfate, filtered and concentrated under reduced pressure to give a residue. The residue was purified by flash chromatography on silica gel to give tert-butyl N-allyl-N-[1-(fluoromethyl)pent-4-ynyl]carbamate (455 mg, 1.78 mmol, 31.97% yield) as light-yellow oil.

¹H NMR (400 MHz, CHLOROFORM-d) δ:1.46 (br s, 9H) 1.81 (br d, J=10.85 Hz, 1 H) 1.86-1.97 (m, 1H) 1.99 (t, J=2.62 Hz, 1H)2.14-2.36 (m, 2H)3.69-3.93 (m, 2H)4.01-4.25 (m, 1H)4.36-4.65 (m, 2H)5.06-5.26 (m, 2H)5.76-5.94 (m, 1H)

### Step 6. tert-butyl N-allyl-N-[1-(fluoromethyl)-4-(4,4,5,5-tetramethyl-1,3,2-dioxaborolan-2-yl)pent-4-enyl]carbamate

To a solution of LiCl (90.65 mg, 2.14 mmol, 43.83 μL) in DMF (15 mL) was added CuCl (211.70 mg, 2.14 mmol), then the mixture was stirred at 20° C. for 3 h under an atmosphere of nitrogen. Then the mixture was added tert-butyl N-allyl-N-[1-(fluoromethyl)pent-4-ynyl]carbamate (455 mg, 1.78 mmol), 4,4,5,5-tetramethyl-2-(4,4,5,5-tetramethyl-1,3,2-dioxaborolan-2-yl)-1,3,2-dioxaborolane (543.03 mg, 2.14 mmol) and AcOK (209.87 mg, 2.14 mmol), then the reaction was stirred at 20° C. for 12 h under an atmosphere of nitrogen. The reaction was added into water (50 mL), then extracted with ethyl acetate (50 mL*3). The combined organic layers were washed with brine (50 mL*3) and dried over anhydrous sodium sulfate, filtered and concentrated under reduced pressure to give a residue. The residue was purified by flash chromatography on silica gel to give tert-butyl N-allyl-N-[1-(fluoromethyl)-4-(4,4,5,5-tetramethyl-1,3,2-dioxaborolan-2-yl)pent-4-enyl]carbamate (310 mg, 808.76 μmol, 45.38% yield) as colorless oil.

¹H NMR (400 MHz, CHLOROFORM-d) δ:1.27 (s, 12H) 1.45-1.50 (m, 9H) 1.63-1.81 (m, 2H) 2.06-2.26 (m, 2H) 3.71-3.92 (m, 2H) 4.04-4.64 (m, 3H) 5.00-5.24 (m, 2H) 5.58-5.67 (m, 1H)5.75-5.93 (m, 2H)

### Step 7. tert-butyl 2-(fluoromethyl)-5-(4,4,5,5-tetramethyl-1,3,2-dioxaborolan-2-yl)-2,3,4,7-tetrahydroazepine-1-carboxylate

To a solution of tert-butyl N-allyl-N-[1-(fluoromethyl)-4-(4,4,5,5-tetramethyl-1,3,2-dioxaborolan-2-yl)pent-4-enyl]carbamate (210 mg, 547.87 μmol) in toluene (45 mL) was added Hoveyda-Grubbs Catalyst 2nd Generation (34.28 mg, 54.79 mol), then the mixture was stirred at 80° C. for 36 h. The reaction was concentrated under reduced pressure to give a residue. The residue was purified by prep-TLC to give tert-butyl 2-(fluoromethyl)-5-(4,4,5,5-tetramethyl-1,3,2-dioxaborolan-2-yl)-2,3,4,7-tetrahydroazepine-1-carboxylate (125 mg, 351.86 μmol, 64.22% yield) as colorless oil.

¹H NMR (400 MHz, CHLOROFORM-d) δ: 1.26 (s, 12H) 1.45 (d, J=9.01 Hz, 9H) 1.75-1.96 (m, 2H) 2.14-2.29 (m, 1H) 2.48-2.60 (m, 1H) 3.62-3.81 (m, 1H) 4.19-4.56 (m, 4H) 6.39-6.54 (m, 1H)

### Step 8. tert-butyl 2-(fluoromethyl)-5-[6-fluoro-5-[[4-methyl-6-(methylamino)pyrimidin-2-yl]amino 1-2,3-dihydrobenzo furan-7-yl]-2,3,4,7-tetrahydroazepine-1-carboxylate

To a solution of tert-butyl 2-(fluoromethyl)-5-(4,4,5,5-tetramethyl-1,3,2-dioxaborolan-2-yl)-2,3,4,7-tetrahydroazepine-1-carboxylate (125 mg, 351.86 μmol), N2-(7-bromo-6-fluoro-2,3-dihydrobenzofuran-5-yl)-N4,6-dimethyl-pyrimidine-2,4-diamine (124.27 mg, 351.86 mol) and Na₂CO₃ (111.88 mg, 1.06 mmol) in toluene (3.5 mL), EtOH (1.5 mL) and H₂O (0.5 mL) was added Pd(PPh₃)₂Cl₂ (24.70 mg, 35.19 μmol), then the mixture was stirred at 100° C. for 12 h under an atmosphere of nitrogen. The reaction was filtered and concentrated under reduced pressure to give tert-butyl 2-(fluoromethyl)-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (176 mg, crude) as a brown solid.

### Step 9. N2-[6-fluoro-7-[rel-(2R)-2-(fluoromethyl)-2,3,4,7-tetrahydro-1H-azepin-5-yl]-2,3-dihydrobenzofuran-5-yl]-N4,6-dimethyl-pyrimidine-2,4-diamine, N2-[6-fluoro-7-[rel-(2S)-2-(fluoromethyl)-2,3,4,7-tetrahydro-1H-azepin-5-yl]-1-2,3-dihydrobenzofuran-5-yl]-N4,6-dimethyl-pyrimidine-2,4-diamine

To a solution of tert-butyl 2-(fluoromethyl)-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (88 mg, 175.45 mol) in DCM (2 mL) was added ZnBr₂ (197.55 mg, 877.25 mol), then the mixture was stirred at 20° C. for 12 h. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give crude product. The crude product was purified by Chiral SFC (Column: DAICEL CHIRALPAK AD(250 mm*30 mm, 10 um); Mobile phase: A for CO₂ and B for IPA(0.1% NH₃H₂O); Gradient: B %=40% isocratic elution mode; Flow rate: 68 g/min) to give crude Product 1 and Product 2.

The crude Product 1 was re-purified by prep-HPLC (TFA condition, column: Phenomenex Luna 80*30 mm*3 um; mobile phase: [water(TFA)-ACN; B %: 1%-30%, 8 min) and Chiral SFC (Column: DAICEL CHIRALPAK AD(250 mm*30 mm, 10 um); Mobile phase: A for CO₂ and B for IPA(0.1% NH₃H₂O); Gradient: B %=35% isocratic elution mode; Flow rate: 70 g/min) to give N2-[6-fluoro-7-[rel-(2R)-2-(fluoromethyl)-2,3,4,7-tetrahydro-1H-azepin-5-yl]-2,3-dihydrobenzofuran-5-yl]-N4,6-dimethyl-pyrimidine-2,4-diamine (3.2 mg, 7.97 μmol, 4.54% yield) (purity: 93.071%, ee: 100%) as a light-yellow solid.

The crude Product 2 was re-purified by prep-HPLC (TFA condition, column: Phenomenex Luna 80*30 mm*3 um; mobile phase: [water(TFA)-ACN; B %: 1%-30%, 8 min) to give N2-[6-fluoro-7-[rel-(2S)-2-(fluoromethyl)-2,3,4,7-tetrahydro-1H-azepin-5-yl]-2,3-dihydrobenzofuran-5-yl]-N4,6-dimethyl-pyrimidine-2,4-diamine (5 mg, 9.72 μmol, 5.54% yield, TFA) (purity: 100%, ee: 94.34%) as a light-yellow solid.

¹H NMR (400 MHz, METHANOL-d₄) δ: 1.59-1.72 (m, 1H) 1.83-1.93 (m, 1H) 2.16 (s, 3H) 2.52-2.70 (m, 2H) 2.86 (s, 3H) 3.18 (br t, J=8.64 Hz, 3H) 3.39-3.47 (m, 1H) 3.53-3.63 (m, 1H) 4.22-4.50 (m, 2H) 4.51-4.59 (m, 2H) 5.79 (s, 1H) 5.97-6.06 (m, 1H) 7.83 (br d, J=7.03 Hz, 1H)

¹H NMR (400 MHz, METHANOL-d₄) δ: 1.92-2.04 (m, 1H) 2.04-2.15 (m, 1H) 2.28 (s, 3H) 2.65-2.75 (m, 1H) 2.81-2.95 (m, 4H) 3.25 (br t, J=8.56 Hz, 2H) 3.77-3.91 (m, 1H) 3.98 (d, J=6.48 Hz, 2H) 4.49-4.68 (m, 3H) 4.68-4.77 (m, 1H) 5.98 (s, 1H) 6.08 (br t, J=5.87 Hz, 1H) 7.40 (br d, J=7.34 Hz, 1H)

### EXAMPLE 78. Synthesis of Compound 381 and Compound 373

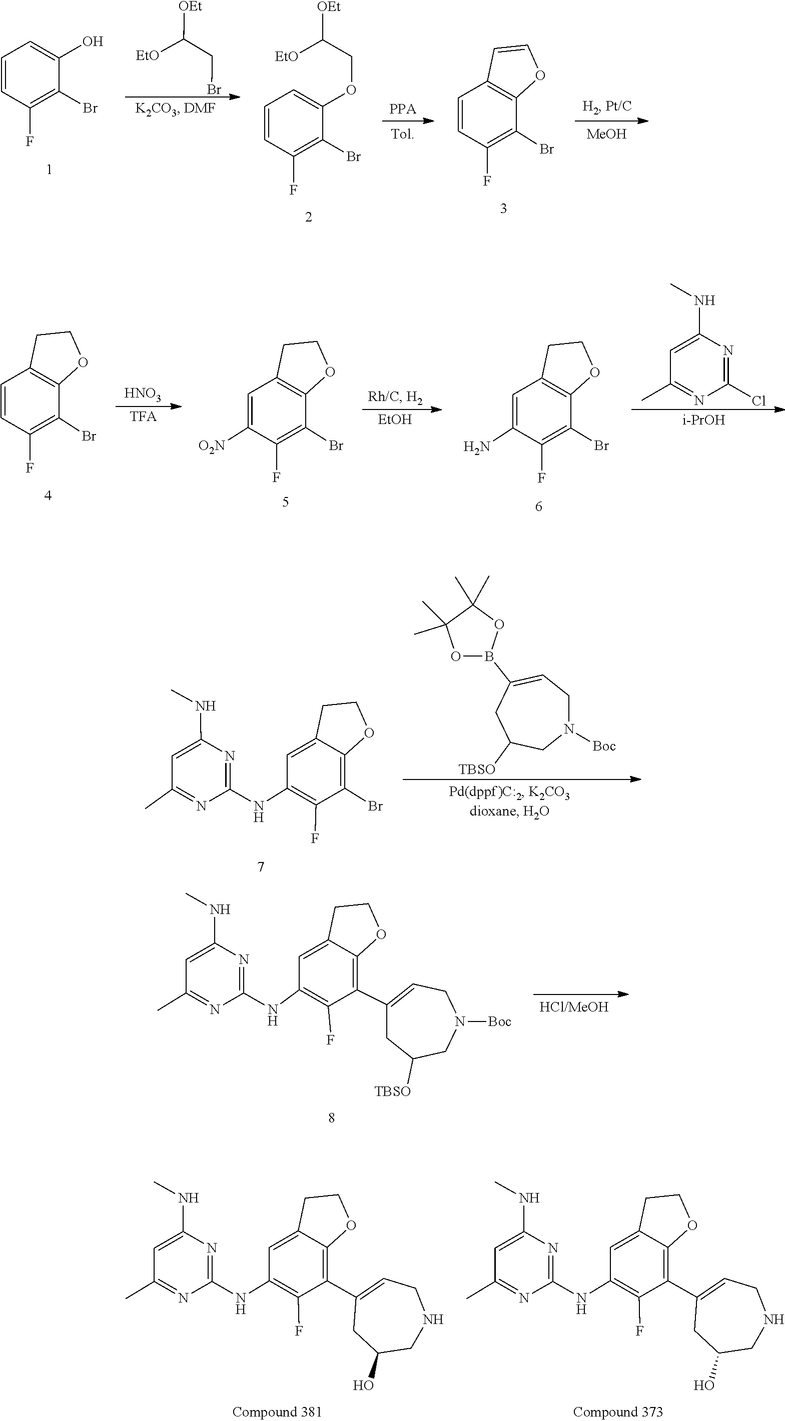

### Step 1.2-bromo-1-(2,2-diethoxyethoxy)-3-fluoro-benzene

2-bromo-3-fluoro-phenol (10 g, 52.36 mmol) was dissolved in DMF (500 mL), and then to the mixture was added K₂CO₃ (10.85 g, 78.54 mmol) and 2-bromo-1,1-diethoxy-ethane (41.27 g, 209.43 mmol), and then the mixture was stirred at 110° C. for 12 h. The 5 batches were combined to work up. The mixture was concentrated to give a residue which was purified by flash chromatography on silica gel. 2-bromo-1-(2,2-diethoxyethoxy)-3-fluoro-benzene (80 g, crude) was obtained as a yellow oil.

### Step 2. 7-bromo-6-fluoro-benzofuran

2-bromo-1-(2,2-diethoxyethoxy)-3-fluoro-benzene (16 g, 52.09 mmol) was dissolved in toluene (800 mL), and then to the mixture was added PPA (40 g, 52.09 mmol), and then the mixture was stirred at 110° C. for 2 h. The mixture was concentrated in vacuum. The five reactions were work up together. The residue was purified by flash chromatography on silica gel to give 7-bromo-6-fluoro-benzofuran (30 g, crude) as a yellow oil.

### Step 3. 7-bromo-6-fluoro-2,3-dihydrobenzofuran

7-bromo-6-fluoro-benzofuran (8 g, 37.21 mmol) was dissolved in EtOH (400 mL) and then to the mixture was added Pt\C (10 g, 5% purity), and then the mixture was stirred at 25° C. for 3 hrs under H₂ (37.21 mmol) (15 psi). The four reactions were work up together. The mixture was filtered, and the filtrated was concentrated to give 7-bromo-6-fluoro-2,3-dihydrobenzofuran (20 g, crude) as a yellow solid.

### Step 4. 7-bromo-6-fluoro-5-nitro-2,3-dihydrobenzofuran

7-bromo-6-fluoro-2,3-dihydrobenzofuran (20 g, 92.15 mmol) was dissolved in TFA (200 mL), and then to the mixture was added HNO₃ (8.71 g, 138.23 mmol, 1 mL) at 0° C.-10° C., and the mixture was stirred at 25° C. for 1 h. The suspension was added to ice (200 mL), and then the suspension was filtered, and the filter cake was washed with H₂O (30 mL*3), dried in vacuum to give 7-bromo-6-fluoro-5-nitro-2,3-dihydrobenzofuran (24 g, 91.59 mmol, 99.39% yield) as a yellow solid.

### Step 5. 7-bromo-6-fluoro-2,3-dihydrobenzofuran-5-amine

7-bromo-6-fluoro-5-nitro-2,3-dihydrobenzofuran (4 g, 15.27 mmol) was dissolved in EtOH (500 mL) and then to the mixture was added Rh/C (6.00 g, 2.54 mmol, 5% purity), the reaction mixture was stirred at 25° C. for 2 hrs under H₂ (15 psi). The three reactions were work up together. The mixture was filtered, and the filtrated was concentrated in vacuum to give 7-bromo-6-fluoro-2,3-dihydrobenzofuran-5-amine (5.6 g, crude) as a yellow solid.

### Step 6. N2-(7-bromo-6-fluoro-2,3-dihydrobenzofuran-5-yl)-N4,6-dimethyl-pyrimidine-2,4-diamine

To the mixture of 7-bromo-6-fluoro-2,3-dihydrobenzofuran-5-amine (250 mg, 1.08 mmol),2-chloro-N,6-dimethyl-pyrimidin-4-amine (203.75 mg, 1.29 mmol) in i-PrOH (10 mL) was added TFA (0.1 mL), then the reaction mixture was stirred at 130° C. for 2 hrs. The four reactions was work up together. The reaction was concentrated under reduced pressure to give N2-(7-bromo-6-fluoro-2,3-dihydrobenzofuran-5-yl)-N4,6-dimethyl-pyrimidine-2,4-diamine (2 g, crude) as brown solid.

### Step 7. tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate

To the mixture of tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (256.80 mg, 566.27 μmol) and N2-(7-bromo-6-fluoro-2,3-dihydrobenzofuran-5-yl)-N4,6-dimethyl-pyrimidine-2,4-diamine (200 mg, 566.27 μmol) in dioxane (5 mL) and H₂O (0.5 mL) was added K₂CO₃ (234.78 mg, 1.70 mmol) and cyclopentyl(diphenyl)phosphane;dichloropalladium;iron (82.87 mg, 113.25 μmol), then the reaction mixture was stirred at 100° C. for 12 hrs. The reaction was concentrated under reduced pressure to give a residue. The residue was purified by prep-TLC to give tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (150 mg, crude) as yellow oil.

### Step 8. rel-(3S)-5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol and rel-(3R)-5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol

A solution of tert-butyl 3-[tert-butyl(dimethyl)silyl]oxy-5-[6-fluoro-5-[[4-methyl-6-(methylamino)pyrimidin-2-yl]amino]-2,3-dihydrobenzofuran-7-yl]-2,3,4,7-tetrahydroazepine-1-carboxylate (30 mg, 50.02 mol) in HCl/MeOH (1 mL) was stirred at 25° C. for 1 hrs. The reaction was filtered and concentrated under reduced pressure to give a residue which was purified by prep-HPLC(TFA condition:column:Phenomenex Luna 80*30 mm*3 um; mobile phase:water(TFA)-ACN; B %: 1%-25%,8 min) to give a residue. The residue was separated by Chiral SFC (column: DAICEL CHIRALCEL OZ 250*25 mm I.D. 10 um; mobile phase: A for Heptane and B for EtOH(0.1% IPA); Gradient: B %=40%; SFC: Acq Method: IE_H_I_ACN_IPAm_30_13_30) to give P1 (Rt=4.155 min) (SFC Method:OZ10_H_E_IB_40_10_MS) and P2 (Rt=5.153 min) (SFC; Method: OZ10_H_E_IB_40_10_MS).

rel-(3S)-5-[6-fluoro-5-[[4-methyl-6-(methylamino)pyrimidin-2-yl]amino]-2,3 dihydrobenzofuran-7-yl]-2,3,4,7-tetrahydro-1H-azepin-3-ol (9.1 mg, 23.61 μmol, 47.20% yield) was obtained as brown solid and rel-(3R)-5-[6-fluoro-5-[[4-methyl-6-(methylamino)pyrimidin-2-yl]amino]-2,3-dihydrobenzofuran-7-yl]-2,3,4,7-tetrahydro-1H-azepin-3-ol (9.6 mg, 24.91 μmol, 49.80% yield) was obtained as brown solid.

¹H NMR (400 MHz, METHANOL-d₄) δ: 7.84 (br d, J=7.3 Hz, 1H), 6.09 (br t, J=5.6 Hz, 1H), 5.81-5.75 (m, 1H), 4.62-4.51 (m, 2H), 3.93-3.84 (m, 1H), 3.51-3.33 (m, 3H), 3.19 (br t, J=8.6 Hz, 2H), 2.92-2.81 (m, 5H), 2.71-2.61 (m, 1H), 2.21-2.12 (m, 3H)

¹H NMR (400 MHz, METHANOL-d₄) δ: 7.89-7.79 (m, 1H), 6.14-6.05 (m, 1H), 5.85-5.77 (m, 1H), 4.62-4.49 (m, 2H), 3.99-3.85 (m, 1H), 3.36 (br d, J=2.8 Hz, 3H), 3.23-3.15 (m, 2H), 2.93-2.82 (m, 5H), 2.73-2.62 (m, 1H), 2.20-2.11 (m, 3H)

### EXAMPLE 79. Synthesis of Compound 374 and Compound 374-a

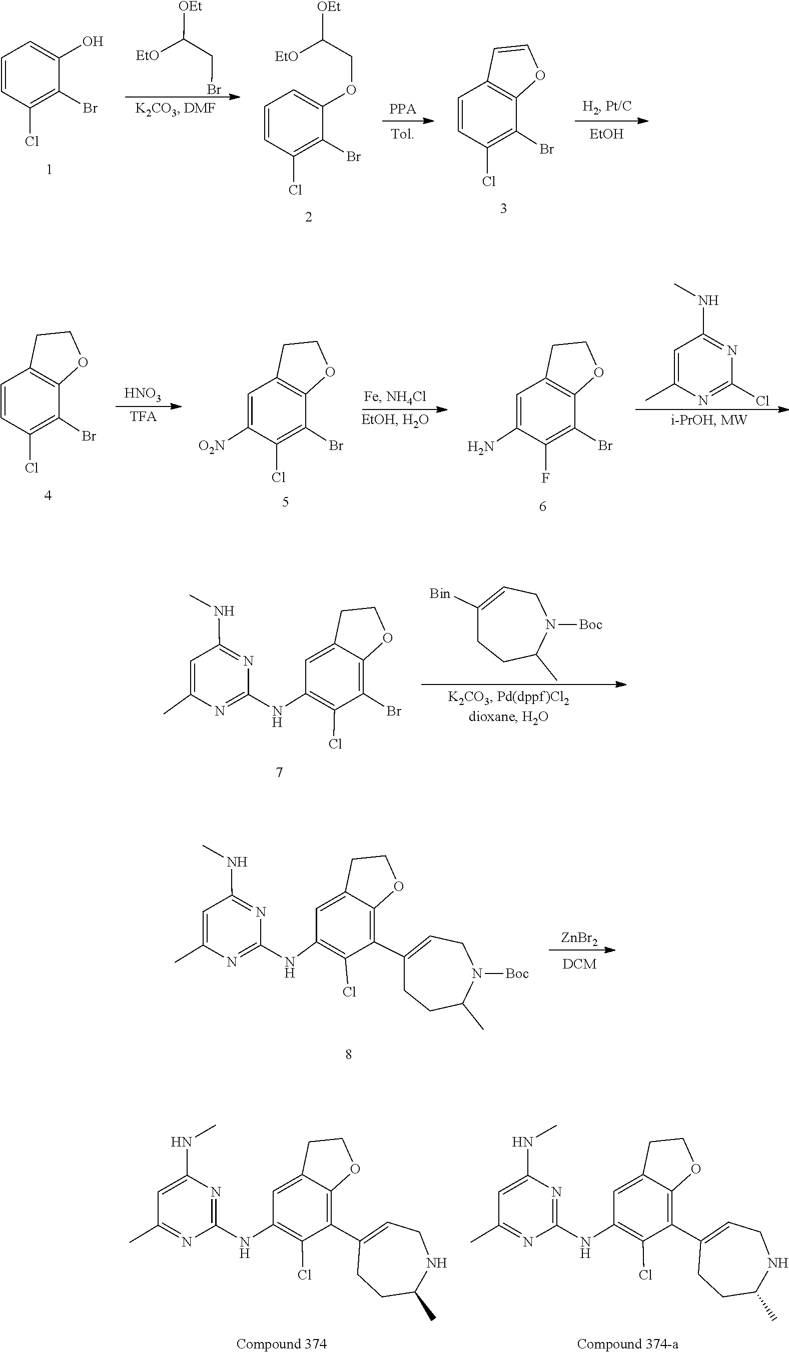

### Step 1. 2-bromo-1-chloro-3-(2,2-diethoxyethoxy)benzene

To a solution of 2-bromo-3-chloro-phenol (25 g, 120.51 mmol) in DMF (250 mL), and then to the mixture was added K₂CO₃ (24.98 g, 180.76 mmol) and 2-bromo-1,1-diethoxy-ethane (95.00 g, 482.04 mmol), and then the mixture was stirred at 110° C. for 12 hrs. The mixture was concentrated to give a residue which was purified by flash column chromatography on silica gel. 2-bromo-1-chloro-3-(2,2-diethoxyethoxy)benzene (30 g, crude) was obtained as a yellow oil.

### Step 2. 7-bromo-6-chloro-benzofuran

To a solution of 2-bromo-1-chloro-3-(2,2-diethoxyethoxy)benzene (5 g, 15.45 mmol) in Tol. (100 mL), and then to the mixture was added PPA (20 g, 15.45 mmol), and then the mixture was stirred at 110° C. for 2 hrs. The mixture was concentrated to remove most of toluene and then dissolved in water (100 mL) and extracted with EtOAc (200 mL*3) and dried over anhydrous sodium sulfate, filtered and concentrated under reduced pressure to give crude product. The residue was purified by flash column chromatography on silica gel to give 7-bromo-6-chloro-benzofuran (6 g, crude) as yellow oil.

### Step 3. 7-bromo-6-chloro-2,3-dihydrobenzofuran

To a solution of 7-bromo-6-chloro-benzofuran (6 g, 25.92 mmol) in EtOH (400 mL) and then to the mixture was added Pt\C (5 g, 5% purity), and then the mixture was stirred at 25° C. for 12 hrs under H₂ (15 psi). The mixture was filtered on celite, and the filtrated was concentrated in vacuum to give 7-bromo-6-chloro-2,3-dihydrobenzofuran (6 g, crude) as a yellow solid.

### Step 4. 7-bromo-6-chloro-5-nitro-2,3-dihydrobenzofuran

To a solution of 7-bromo-6-chloro-2,3-dihydrobenzofuran (500 mg, 2.14 mmol) in H₂SO₄ (210.03 mg, 2.14 mmol, 5 mL) was added HNO₃ (148.43 mg, 2.36 mmol) at 0° C., then the mixture was stirred at 25° C. for 3 hrs. The reaction mixture was poured into crushed ice water (30 mL) with care, extracted with Ethyl acetate (30 mL*5). The organic layer was dried over anhydrous Na₂SO₄ and the mixture was filtered, concentrated under reduced pressure to give a 7-bromo-6-chloro-5-nitro-2,3-dihydrobenzofuran (500 mg, crude) as brown oil.

### Step 5. 7-bromo-6-chloro-2,3-dihydrobenzofuran-5-amine

To a solution of 7-bromo-6-chloro-5-nitro-2,3-dihydrobenzofuran (500 mg, 1.80 mmol) and NH₄Cl (960.39 mg, 17.95 mmol) in H₂O (4 mL) and EtOH (20 mL) was added Fe (501.33 mg, 8.98 mmol), and then the mixture was stirred at 80° C. for 4 hrs. The reaction was filtered and concentrated under reduced pressure to remove most of EtOH and then dissolved in water (25 mL), extracted with ethyl acetate (20 mL*3) and dried over anhydrous sodium sulfate, filtered and concentrated under reduced pressure to give 7-bromo-6-chloro-2,3-dihydrobenzofuran-5-amine (400 mg, crude) as a brown oil.

### Step 6. N2-(7-bromo-6-chloro-2,3-dihydrobenzofuran-5-yl)-N4,6-dimethyl-pyrimidine-2,4-diamine

To a solution of 7-bromo-6-chloro-2,3-dihydrobenzofuran-5-amine (400 mg, 1.61 mmol) and 2-chloro-N,6-dimethyl-pyrimidin-4-amine (253.68 mg, 1.61 mmol) in i-PrOH (1.88 mL) was added TFA (183.54 mg, 1.61 mmol, 124.01 μL), then the mixture was stirred at 140° C. in the microwave for 2 hrs. The mixture was filtered and the filter cake was dried in vacuum to give N2-(7-bromo-6-chloro-2,3-dihydrobenzofuran-5-yl)-N4,6-dimethyl-pyrimidine-2,4-diamine (100 mg, crude) as a brown solid.

### Step 7. tert-butyl 5-[6-chloro-5-[[4-methyl-6-(methylamino)pyrimidin-2-yl]amino]-2,3-dihydrobenzofuran-7-yl]-2-methyl-2,3,4,7-tetrahydroazepine-1-carboxylate

To a mixture of tert-butyl 2-methyl-5-(4,4,5,5-tetramethyl-1,3,2-dioxaborolan-2-yl)-2,3,4,7-tetrahydroazepine-1-carboxylate (18.25 mg, 54.11 μmol) and N2-(7-bromo-6-chloro-2,3-dihydrobenzofuran-5-yl)-N4,6-dimethyl-pyrimidine-2,4-diamine (20 mg, 54.11 mol) in H₂O (0.1 mL) and dioxane (1 mL) was added K₂CO₃ (14.96 mg, 108.21 mol) and cyclopentyl(diphenyl)phosphane:dichloromethane;dichloropalladium:iron (4.42 mg, 5.41 μmol) under N₂ atmosphere. The reaction mixture was stirred at 100° C. for 12 hrs under N2 atmosphere. The reaction was filtered and concentrated under reduced pressure to give tert-butyl 5-[6-chloro-5-[[4-methyl-6-(methylamino)pyrimidin-2-yl]amino]-2,3-dihydrobenzofuran-7-yl]-2-methyl-2,3,4,7-tetrahydroazepine-1-carboxylate (10 mg, crude) as a brown oil which was used for next step directly without further purification.

### Step 8. N2-[6-chloro-7-[rel-(2S)-2-methyl-2,3,4,7-tetrahydro-1H-azepin-5-yl]-2,3-dihydrobenzofuran-5-yl]-N4,6-dimethyl-pyrimidine-2,4-diamine and N2-[6-chloro-7-[rel-(2R)-2-methyl-2,3,4,7-tetrahydro-1H-azepin-5-yl]-2,3-dihydrobenzofuran-5-yl]-N4,6-dimethyl-pyrimidine-2,4-diamine

To a solution of tert-butyl 5-[6-chloro-5-[[4-methyl-6-(methylamino)pyrimidin-2-yl]amino]-2,3-dihydrobenzofuran-7-yl]-2-methyl-2,3,4,7-tetrahydroazepine-1-carboxylate (80 mg, 159.99 μmol) in DCM (2 mL) was added ZnBr₂ (180.15 mg, 799.95 μmol) stirred at 30° C. for 4 hrs. The reaction was concentrated under reduced pressure to give a residue. The residue was purified by prep-HPLC (TFA condition, column: Phenomenex Luna C18 75*30 mm*3 um; mobile phase: [water(TFA)-ACN; B %: 1%-30%, 8 min) to give crude product. The crude product was separated by SFC (Column: DAICEL CHIRALPAK IG(250 mm*30 mm,10 um); Mobile phase: A for CO₂ and B for IPA(0.1% NH₃H₂O); Gradient: B %=45% isocratic elution mode;) to give N2-[6-chloro-7-[rel-(2S)-2-methyl-2,3,4,7-tetrahydro-1H-azepin-5-yl]-2,3-dihydrobenzofuran-5-yl]-N4,6-dimethyl-pyrimidine-2,4-diamine (16.9 mg, 42.26 μmol, 26.41% yield) as a pale yellow solid and N2-[6-chloro-7-[rel-(2R)-2-methyl-2,3,4,7-tetrahydro-1H-azepin-5-yl]-2,3-dihydrobenzofuran-5-yl]-N4,6-dimethyl-pyrimidine-2,4-diamine (23.9 mg, 59.76 μmol, 37.35% yield) as a pale yellow solid.

¹H NMR (400 MHz, CD₃OD, 298 K) δ: 8.04 (br s, 1H), 5.82 (s, 1H), 5.81-5.77 (m, 1H), 4.54 (t, J=8.8 Hz, 2H), 3.58-3.42 (m, 2H), 3.22 (t, J=8.6 Hz, 2H), 3.09 (ddd, J=3.1, 6.3, 9.2 Hz, 1H), 2.87 (s, 3H), 2.64-2.55 (m, 1H), 2.39 (br dd, J=7.0, 16.0 Hz, 1H), 2.17 (s, 3H), 1.95 (tdd, J=2.8, 5.4, 11.3 Hz, 1H), 1.76-1.64 (m, 1H), 1.19 (d, J=6.5 Hz, 3H)

¹H NMR (400 MHz, CD₃OD, 298 K) δ: 7.77 (br s, 1H), 5.92 (s, 1H), 5.88 (dt, J=1.9, 6.1 Hz, 1H), 4.65-4.58 (m, 2H), 3.89 (d, J=6.2 Hz, 2H), 3.63-3.55 (m, 1H), 3.46-3.43 (m, 2H), 3.26 (br t, J=8.7 Hz, 2H), 2.88 (s, 3H), 2.85-2.76 (m, 1H), 2.51 (br dd, J=6.6, 15.9 Hz, 1H), 2.23 (s, 3H), 2.13-2.06 (m, 1H), 2.00-1.90 (m, 1H), 1.40 (d, J=6.6 Hz, 3H)

### EXAMPLE 80. Synthesis of Compound 384 and Compound 376

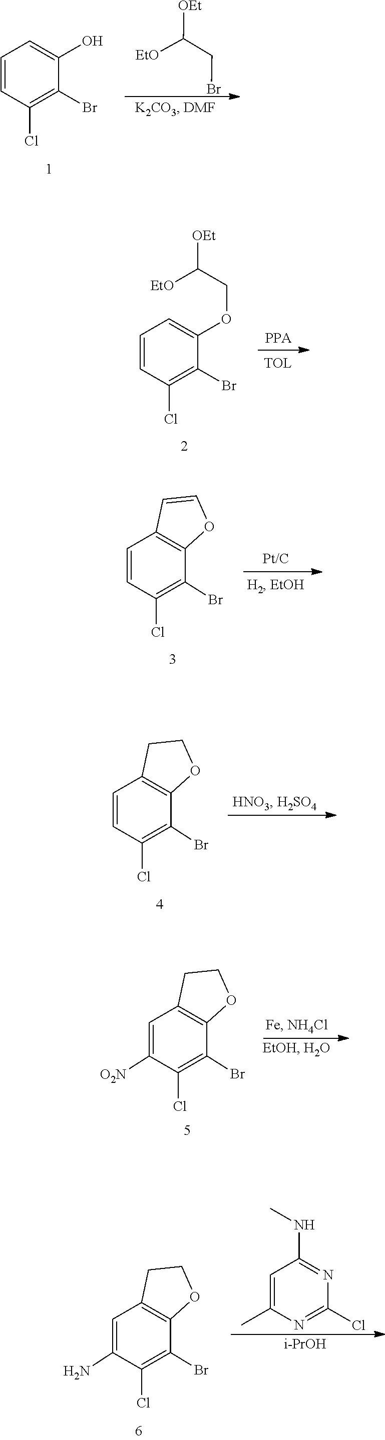

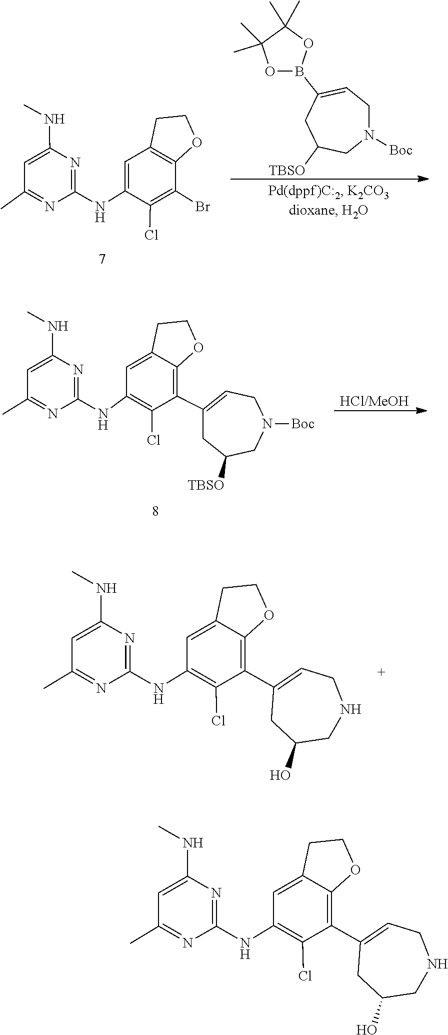 [SMILES: CNc1cc(C)nc(Nc2cc3c(c(Br)c2Cl)OCC3)n1 ; CC(C)(C)OC(=O)N1CC=C(B2OC(C)(C)C(C)(C)O2)CC(O[Si](C)(C)C(C)(C)C)C1 ; CNc1cc(C)nc(Nc2cc3c(c(C4=CCN(C(=O)OC(C)(C)C)C[C@@H](O[Si](C)(C)C(C)(C)C)C4)c2Cl)OCC3)n1 ; "HCl/MeOH" ; CNc1cc(C)nc(Nc2cc3c(c(C4=CCNC[C@@H](O)C4)c2Cl)OCC3)n1 ; CNc1cc(C)nc(Nc2cc3c(c(C4=CCNC[C@H](O)C4)c2Cl)OCC3)n1]

### Step 1. 2-bromo-1-chloro-3-(2,2-diethoxyethoxy)benzene

K₂CO₃ (24.98 g, 180.76 mmol) was added to the mixture of 2-bromo-3-chloro-phenol (25 g, 120.51 mmol) and 2-bromo-1,1-diethoxy-ethane (95.00 g, 482.04 mmol) in DMF (250 mL), and then the mixture was stirred at 110° C. for 12 h. The mixture was added to water (500 mL), extracted with EtOAc (300 mL*3), the organic layer was washed with brine (200 mL), dried over Na₂SO₄, concentrated to give a residue. The residue was purified by flash chromatography on silica gel to give 2-bromo-1-chloro-3-(2,2-diethoxyethoxy)benzene (30 g, crude) as yellow oil.

### Step 2. 7-bromo-6-chloro-benzofuran

2-bromo-1-chloro-3-(2,2-diethoxyethoxy)benzene (5 g, 15.45 mmol) was dissolved in Tol (100 mL), and then to the mixture was added PPA (20 g, 15.45 mmol), after addition the mixture was stirred at 110° C. for 2 h. The reaction mixture was quenched by water (300 mL) and extracted with EtOAc (200 mL*3) and dried over anhydrous sodium sulfate, filtered and concentrated under reduced pressure to give crude product. The residue was purified by flash chromatography on silica gel to give 7-bromo-6-chloro-benzofuran (6 g, crude) as yellow oil.

### Step 3. 7-bromo-6-chloro-2,3-dihydrobenzofuran

7-bromo-6-chloro-benzofuran (6 g, 25.92 mmol) was dissolved in EtOH (400 mL) and then to the mixture was added Pt\C (5 g, 5% purity), and then the mixture was stirred at 25° C. for 12 hrs under H₂ (15 psi). The mixture was filtered and the filtrate was concentrated in vacuum to give 7-bromo-6-chloro-2,3-dihydrobenzofuran (6 g, crude) as a yellow solid.

### Step 4. 7-bromo-6-chloro-5-nitro-2,3-dihydrobenzofuran

To a solution of 7-bromo-6-chloro-2,3-dihydrobenzofuran (3.9 g, 16.70 mmol) in H₂SO₄ (1.64 g, 16.70 mmol) was added HNO₃ (1.70 g, 18.37 mmol, 68% purity) at 0° C., then the mixture was stirred at 25° C. for 3 hrs. The reaction mixture was poured into crushed ice water (300 mL) extracted with Ethyl acetate (100 mL*5), The organic layer was dried over anhydrous Na₂SO₄ and the mixture was filtered, concentrated under reduced pressure to give a 7-bromo-6-chloro-5-nitro-2,3-dihydrobenzofuran (3.72 g, crude) as brown oil.

### Step 5. 7-bromo-6-chloro-2,3-dihydrobenzofuran-5-amine

To a mixture of 7-bromo-6-chloro-5-nitro-2,3-dihydrobenzofuran (3.72 g, 13.36 mmol) in H₂O (30 mL), EtOH (150 mL) was added Fe (3.73 g, 66.79 mmol), NH₄Cl (7.15 g, 133.58 mmol) at 25° C., then the mixture was stirred at 80° C. for 12 hrs. The reaction mixture was filtered and the filtrate was concentrated to give the crude product. The crude product was added to water (100 mL), extracted with EtOAc (100 mL*3). The combined organic layers were washed with 10 mL of brine, dried over Na₂SO₄, filtered and concentrated under reduced pressure to give the crude product. The crude product was purified by column chromatography on silica gel to give 7-bromo-6-chloro-2,3-dihydrobenzofuran-5-amine (2.5 g, 10.06 mmol, 75.31% yield) as yellow solid.

### Step 6. N2-(7-bromo-6-chloro-2,3-dihydrobenzofuran-5-yl)-N4,6-dimethyl-pyrimidine-2,4-diamine

To a solution of 7-bromo-6-chloro-2,3-dihydrobenzofuran-5-amine (200 mg, 804.82 μmol) and 2-chloro-N,6-dimethyl-pyrimidin-4-amine (126.84 mg, 804.82 μmol) in i-PrOH (1 mL) was added TFA (91.77 mg, 804.82 mol, 62.00 μL), then the mixture was stirred at 140° C. for 2 hrs. The mixture was filtered and the filter cake was dried in vacuum to give N2-(7-bromo-6-chloro-2,3-dihydrobenzofuran-5-yl)-N4,6-dimethyl-pyrimidine-2,4-diamine (260 mg, crude) as a brown soild.

### Step 7. tert-butyl 3-[tert-butyl(dimethyl)silyl]oxy-5-[6-chloro-5-[[4-methyl-6-(methylamino)-pyrimidin-2-yl]amino]-2,3-dihydrobenzofuran-7-yl]-2,3,4,7-tetrahydroazepine-lcarboxylate

To a mixture of N2-(7-bromo-6-chloro-2,3-dihydrobenzofuran-5-yl)-N4,6-dimethyl-pyrimidine-2,4-diamine (60 mg, 162.32 μmol) and tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (73.61 mg, 162.32 μmol) in dioxane (3 mL) and H₂O (0.3 mL) was added K₂CO₃ (44.87 mg, 324.64 μmol) and Pd(dppf)Cl₂ (13.26 mg, 16.23 μmol) under N₂ atmosphere. The reaction mixture was stirred at 100° C. for 12 hrs under N₂ atmosphere. The reaction was filtered and concentrated under reduced pressure to give a residue. The reside was purified by prep-TLC to give tert-butyl 3-[tert-butyl(dimethyl)silyl]oxy-5-[6-chloro-5-[[4-methyl-6-(methylamino)-pyrimidin-2-yl]amino]-2,3-dihydrobenzofuran-7-yl]-2,3,4,7-tetrahydroazepine-1-carboxylate (120 mg, crude) as brown oil.

### Step 8. rel-(3S)-5-[6-chloro-5-[4-methyl-6-(methylamino)pyrimidin-2-yl]amino]-2,3-dihydrobenzo furan-7-yl]-2,3,4,7-tetrahydro-1H-azepin-3-ol and rel-(3R)-5-46-chloro-5-[[4-methyl-6-(methylamino)pyrimidin-2-yl]amino]-2,3-dihydrobenzofuran-7-yl]-2,3,4,7-tetrahydro-1H-azepin-3-ol

A solution of tert-butyl 3-[tert-butyl(dimethyl)silyl]oxy-5-[6-chloro-5-[[4-methyl-6-(methylamino)pyrimidin-2-yl]amino]-2,3-dihydrobenzofuran-7-yl]-2,3,4,7-tetrahydroazepine-1-carboxylate (0.124 g, 201.21 mol) in HCl/MeOH (4.1 mL) was stirred at 20° C. for 2 hrs. The reaction mixture was concentrated and purified by prep-HPLC (TFA condition:column: Phenomenex Luna 80*30 mm*3 um; mobile phase:water(TFA)-ACN; B %: 1%-25%,8 min) to give the mixture of these two single isomer. The mixture was separated by SFC (column: DAICEL CHIRALPAKIC (250 mm*30 mm,10 um); mobile phase:[0.1% NH₃H₂O MEOH]; B %: 55%-55%; SFC, Acq Method: IC_MeOH_IPAm_5_50_3_35_3 min) to give rel-(3S)-5-[6-chloro-5-[[4-methyl-6-(methylamino)pyrimidin-2-yl]amino]-2,3-dihydrobenzofuran-7-yl]-2,3,4,7-tetrahydro-1H-azepin-3-ol (5 mg, 12.44 μmol, 6.18% yield, Rt=2.157 min) as a white solid and rel-(3R)-5-[6-chloro-5-[[4-methyl-6-(methylamino)pyrimidin-2-yl]amino]-2,3 dihydrobenzofuran-7-yl]-2,3,4,7-tetrahydro-1H-azepin-3-ol (5.2 mg, 12.94 μmol, 6.43% yield, Rt=2.455 min) as a white solid.

¹H NMR (400 MHz, METHANOL-d₄) δ: 2.15 (s, 6H) 2.49-2.62 (m, 1H) 2.81 (s, 3H) 2.91 (br dd, J=13.32, 8.44 Hz, 1H) 3.19 (s, 2H) 3.29-3.35 (m, 1H) 3.31 (dt, J=3.25, 1.63 Hz, 22H) 3.39-3.54 (m, 2H) 3.89-4.06 (m, 1H) 4.53 (br d, J=1.38 Hz, 1H) 4.86-4.87 (m, 1H) 4.86 (s, 24H) 5.73-5.76 (m, 1H) 5.80 (br s, 1H) 7.25 (s, 1H)

¹H NMR (400 MHz, METHANOL-d₄) δ: 2.14 (s, 6H) 2.51-2.61 (m, 1H) 2.80 (s, 3H) 2.83-2.93 (m, 1H) 3.13-3.22 (m, 2H) 3.38-3.51 (m, 2H) 3.88-4.03 (m, 1H) 4.47-4.56 (m, 2H)5.72-5.74 (m, 1H)5.75-5.82 (m, 1H)_{7.22}-7.28 (m, 1H).

### EXAMPLE 81. Synthesis of Compound 383-a and Compound 383

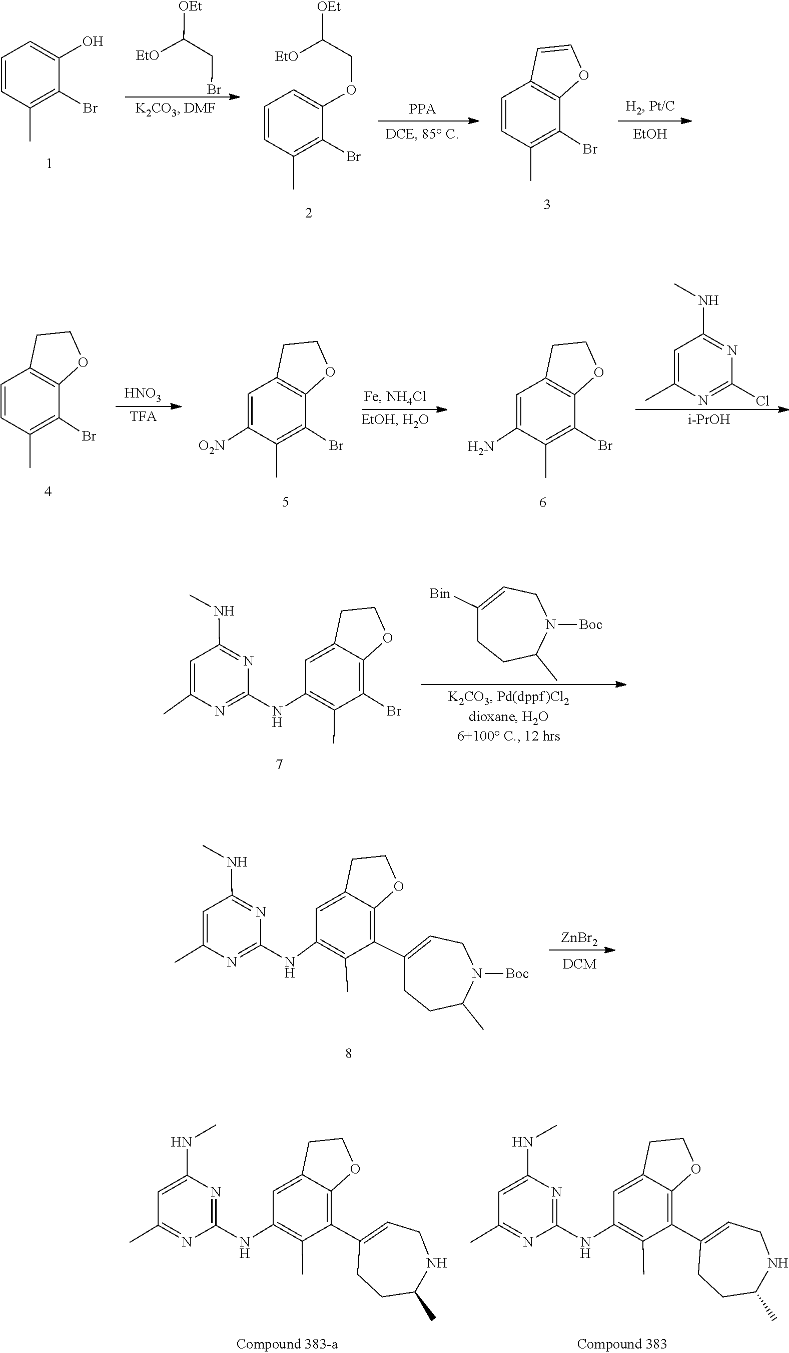

### Step 1. 2-bromo-1-(2,2-diethoxyethoxy)-3-methyl-benzene

To a solution of 2-bromo-3-methyl-phenol (5 g, 26.73 mmol) in DMF (50 mL) was added NaH (1.60 g, 40.10 mmol, 784.66 L, 60% purity) at 0° C. The mixture was stirred at 0° C. for 0.5 h. Then 2-bromo-1,1-diethoxy-ethane (6.32 g, 32.08 mmol) was added to the above mixture at 0° C. After addition, the reaction mixture was stirred at 80° C. for 12 h. The reaction was added into NH₄Cl solution (50 mL), then extracted with ethyl acetate (100 ml*3). The combined organic layers were washed with brine (50 mL*3) and dried over anhydrous sodium sulfate and concentrated under reduced pressure to give a residue. The residue was purified by flash column chromatography on silica gel to give 2-bromo-1-(2,2-diethoxyethoxy)-3-methyl-benzene (7 g, crude) as a yellow oil.

### Step 2. 7-bromo-6-methyl-benzofuran

2-bromo-1-(2,2-diethoxyethoxy)-3-methyl-benzene (7 g, 23.09 mmol) was dissolved in toluene (220 mL), and then to the mixture was added PPA (15 g, 23.09 mmol), and then the mixture was stirred at 110° C. for 2 h. The mixture was concentrated in vacuum. The residue was purified by flash column chromatography on silica gel to give 7-bromo-6-methyl-benzofuran (1.8 g, 8.53 mmol) as a yellow oil.

### Step 3. 7-bromo-6-methyl-2,3-dihydrobenzofuran

7-bromo-6-methyl-benzofuran (1.8 g, 8.53 mmol) was dissolved in EtOH (180 mL) and then to the mixture was added Pt\C (179.19 mg, 852.86 mol), and then the mixture was stirred at 25° C. for 12 hrs under H₂ (17.19 mg, 8.53 mmol) (15 psi). The reaction mixture was filtered and the filtrate was concentrated to give 7-bromo-6-methyl-2,3-dihydrobenzofuran (1.3 g, 6.10 mmol, crude) as a yellow solid.

### Step 4.7-bromo-6-methyl-5-nitro-2,3-dihydrobenzofuran

7-bromo-6-methyl-2,3-dihydrobenzofuran (1.3 g, 6.10 mmol) was dissolved in TFA (14 mL), and then to the mixture was added HNO₃ (848.07 mg, 9.15 mmol, 68% purity) at 0° C.-10° C., and the mixture was stirred at 25° C. for 1 hr. The suspension was added to ice (200 mL), and then the suspension was filtered, and the filter cake was washed with H₂O (30 mL*3), and then concentrated in vacuum. 7-bromo-6-methyl-5-nitro-2,3-dihydrobenzofuran (1 g, 3.87 mmol) was obtained as a yellow solid.

### Step 5. 7-bromo-6-methyl-2,3-dihydrobenzofuran-5-amine

To a mixture of 7-bromo-6-methyl-5-nitro-2,3-dihydrobenzofuran (1 g, 3.87 mmol) in EtOH (20 mL), H₂O (2 mL) was added Fe (1.08 g, 19.37 mmol), NH₄Cl (2.07 g, 38.75 mmol) at 25° C., then the mixture was stirred at 80° C. for 12 hrs. The reaction mixture was filtered and concentrated to remove most of EtOH and then added to water (100 mL), extracted with EtOAc (50 mL*3). The combined organic layers were washed with 10 mL of brine, dried over anhydrous Na₂SO₄, filtered and concentrated under reduced pressure to give the crude product. The crude product was purified by prep-TLC to give 7-bromo-6-methyl-2,3-dihydrobenzofuran-5-amine (0.9 g, crude) as a yellow solid.

### Step 6. N2-(7-bromo-6-methyl-2,3-dihydrobenzofuran-5-yl)-N4,6-dimethyl-pyrimidine-2,4-diamine

To a solution of 7-bromo-6-methyl-2,3-dihydrobenzofuran-5-amine (200 mg, 876.86 μmol) and 2-chloro-N,6-dimethyl-pyrimidin-4-amine (138.19 mg, 876.86 mol) in i-PrOH (4 mL) was added TFA (10 mg), then the mixture was stirred at 140° C. for 2 hrs. The reaction was concentrated under reduced pressure to give N2-(7-bromo-6-methyl-2,3-dihydrobenzofuran-5-yl)-N4,6-dimethyl-pyrimidine-2,4-diamine (240 mg, crude) as brown soild.

### Step 7. tert-butyl 2-methyl-5-[6-methyl-5-[[4-methyl-6-(methylamino)pyrimidin-2-ylamino-2,3-dihydrobenzo furan-7-yl]-2,3,4,7-tetrahydroazepine-1-carboxylate

To a mixture of N2-(7-bromo-6-methyl-2,3-dihydrobenzofuran-5-yl)-N4,6-dimethyl-pyrimidine-2,4-diamine(80 mg, 229.08 μmol), tert-butyl2-methyl-5-(4,4,5,5-tetramethyl-1,3,2-dioxaborolan-2-yl)-2,3,4,7-tetrahydroazepine 1carboxylate (77.26 mg, 229.08 μmol) in dioxane (2 mL) and H₂O (0.2 mL) was added K₂CO₃ (63.32 mg, 458.16 μmol) and cyclopentyl(diphenyl)phosphane; Dichloromethane;dichloropalladium;iron (18.71 mg, 22.91 mol) under N₂ atmosphere. The reaction mixture was stirred at 100° C. for 12 h under N2 atmosphere. The reaction was concentrated under reduced pressure to give tert-butyl 2-methyl-5-[6-methyl-5-[[4-methyl-6-(methylamino) pyrimidin-2-yl]amino]-2,3-dihydrobenzofuran-7-yl]-2,3,4,7-tetrahydroazepine-1-carboxylate (109 mg, crude) as a yellow solid.

### Step 8. N4,6-dimethyl-N2-[6-methyl-7-[rel-(2S)-2-methyl-2,3,4,7-tetrahydro-1H-azepin-5-yl]-2,3-dihydrobenzofuran-5-yl]pyrimidine-2,4-diamine and N4,6-dimethyl-N2-[6-methyl-7-[rel-(2R)-2-methyl-2,3,4,7-tetrahydro-1H-azepin-5-yl]-2,3-dihydrobenzofuran-5-yl]pyrimidine-2,4-diamine

To a mixture of tert-butyl 2-methyl-5-[6-methyl-5-[[4-methyl-6-(methylamino)pyrimidin-2-yl]amino]-2,3-dihydrobenzofuran-7-yl]-2,3,4,7 tetrahydroazepine-1-carboxylate (109 mg, 227.27 mol) in DCM (3 mL) was added ZnBr₂ (511.80 mg, 2.27 mmol) at 25° C., then the mixture was stirred at 25° C. for 12 hrs. The reaction mixture was filtered and the filtrate was concentrated and then purified by prep-HPLC (TFA condition:column:Phenomenex Luna 80*30 mm*3 um; mobile phase:water(TFA)-ACN; B %: 1%-30%,8 min) to give the mixture of these two single isomer. The mixture was separated by SFC (column: DAICEL CHIRALPAKIC (250 mm*30 mm,10 um); mobile phase:[0.1% NH₃H₂O MEOH]; B %: 50%-55%; SFC, Acq Method: IC_MeOH_IPAm_5_50_3_35_3 min) to give rel-(3S)-5-[6-chloro-5-[[4-methyl-6-(methylamino)pyrimidin-2-yl]amino]-2,3-dihydrobenzofuran-7-yl]-2,3,4,7-tetrahydro-1H-azepin-3-ol (14 mg, 12.44 μmol, 6.18% yield, Rt=1.242 min) as a white solid and rel-(3R)-5-[6-chloro-5-[[4-methyl-6-(methylamino)pyrimidin-2-yl]amino]-2,3 dihydrobenzofuran-7-yl]-2,3,4,7-tetrahydro-1H-azepin-3-ol (5.2 mg, 12.94 mol, 6.43% yield, Rt=1.735 min) as a white solid.

¹H NMR (400 MHz, METHANOL-d₄) δ: 1.12-1.22 (m, 3H) 1.57-1.74 (m, 1H) 1.87-1.99 (m, 1H) 2.06-2.23 (m, 7H) 2.28-2.41 (m, 1H) 2.49-2.64 (m, 1H) 2.75-2.84 (m, 3H) 3.03-3.10 (m, 1H) 3.13-3.21 (m, 2H) 3.35-3.60 (m, 2H) 4.43-4.55 (m, 2H) 4.86 (s, 27H) 5.63-5.69 (m, 1H) 5.71-5.75 (m, 1H) 7.14-7.30 (m, 1H).

¹HNMR (400 MHz, METHANOL-d₄) δ: 1.13-1.28 (m, 1H) 1.31 (d, J=6.60 Hz, 1H) 1.94-2.09 (m, 2H) 2.09-2.19 (m, 1H) 2.27-2.47 (m, 1H) 2.76 (br s, 1H) 3.12-3.22 (m, 5H) 3.48 (br s, 1H) 3.79 (br d, J=5.87 Hz, 1H) 4.49 (br t, J=8.62 Hz, 1H) 4.72-4.78 (m, 1H) 4.76 (s, 11H) 5.64 (br s, 1H) 5.83 (s, 1H) 7.00-7.07 (m, 1H).

### EXAMPLE 82. Synthesis of Compound 385-a and Compound 385

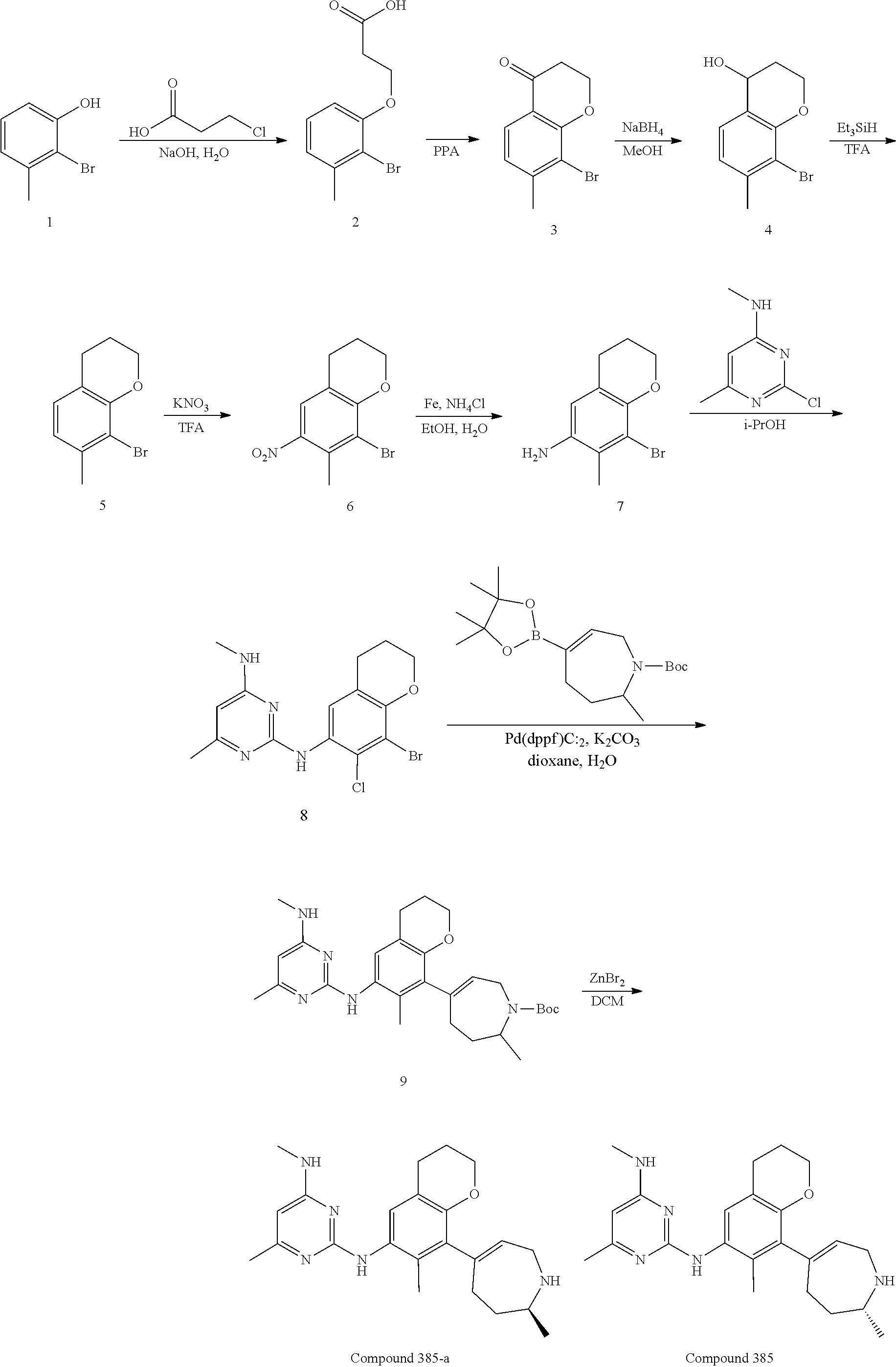

### Step 1. 3-(2-bromo-3-methyl-phenoxy)propanoic acid

A mixture of 2-bromo-3-methyl-phenol (14 g, 74.85 mmol) and 3-chloropropanoic acid (8.12 g, 74.85 mmol, 6.40 mL), then was added dropwise a solution of NaOH (7.49 g, 187.13 mmol, 3.51 mL) in H₂O (45 mL) at 20° C., then the mixture was stirred at 100° C. for 12 h. The reaction was added 3 N HCl to pH=4, then extracted with ethyl acetate (100 mL*3) and dried over anhydrous sodium sulfate, filtered and concentrated under reduced pressure to give a residue. The residue was added ethyl acetate (50 mL), then filtered and the filter cake concentrated under reduced pressure to give 3-(2-bromo-3-methyl-phenoxy)propanoic acid (5.75 g, 22.19 mmol, 29.65% yield) as a white solid.

¹H NMR (400 MHz, METHANOL-d₄) δ: 2.38 (s, 3H) 2.80 (t, J=6.13 Hz, 2H) 4.27 (t, J=6.19 Hz, 2H) 6.88 (t, J=8.50 Hz, 2H) 7.12-7.22 (m, 1H)

### Step 2. 8-bromo-7-methyl-chroman-4-one

A solution of 3-(2-bromo-3-methyl-phenoxy)propanoic acid (5.75 g, 22.19 mmol) in PPA (60 mL), then the mixture was stirred at 100° C. for 2 h. The reaction was added into water (400 mL), then extracted with ethyl acetate (400 mL*3). The combined organic layers were washed with saturated sodium bicarbonate solution (500 mL*3) and dried over anhydrous sodium sulfate, filtered and concentrated under reduced pressure to give a residue. The residue was purified by flash chromatography on silica gel to give 8-bromo-7-methyl-chroman-4-one (4.87 g, 20.20 mmol, 91.02% yield) as an orange solid.

¹H NMR (400 MHz, METHANOL-d₄) δ: 2.44 (s, 3H) 2.78-2.84 (m, 2H) 4.60-4.66 (m, 2H) 6.99 (d, J=8.11 Hz, 1H) 7.71 (d, J=7.99 Hz, 1H)

### Step 3. 8-bromo-7-methyl-chroman-4-ol

To a solution of 8-bromo-7-methyl-chroman-4-one (4.87 g, 20.20 mmol) in MeOH (50 mL) was added NaBH₄ (1.15 g, 30.30 mmol) at 0° C., then the mixture was stirred at 20° C. for 1 h. The reaction was concentrated under reduced pressure and added to water (50 mL), then extracted with ethyl acetate (50 mL*3), dried over anhydrous sodium sulfate, filtered and concentrated under reduced pressure to give 8-bromo-7-methyl-chroman-4-ol (4.9 g, crude) as a green solid.

¹H NMR (400 MHz, METHANOL-d₄) δ: 1.95-2.02 (m, 1H) 2.03-2.12 (m, 1H) 2.35 (s, 3H) 4.25-4.38 (m, 2H) 4.72 (t, J=4.29 Hz, 1H) 6.84 (d, J=7.87 Hz, 1H) 7.19 (d, J=7.75 Hz, 1H)

### Step 4. 8-bromo-7-methyl-chromane

To a solution of 8-bromo-7-methyl-chroman-4-ol (4.9 g, 20.16 mmol) in TFA (50 mL) was added triethylsilane (4.69 g, 40.31 mmol, 6.44 mL), then the mixture was stirred at 60° C. for 12 h. The reaction was concentrated under reduced pressure, then added water (50 mL), then extracted with ethyl acetate (3*50 mL). The combined organic layers were washed with brine (50 mL) and dried over anhydrous sodium sulfate, filtered and concentrated under reduced pressure to give a residue. The residue was purified by flash chromatography on silica gel to give 8-bromo-7-methyl-chromane (2.56 g, 11.27 mmol, 55.93% yield) as a colorless oil.

¹H NMR (400 MHz, METHANOL-d₄) δ: 1.90-1.98 (m, 2H) 2.25 (s, 3H) 2.72 (t, J=6.44 Hz, 2H) 4.01-4.16 (m, 2H) 6.64 (s, 1H) 7.16 (s, 1H)

### Step 5. 8-bromo-7-methyl-6-nitro-chromane

To a solution of 8-bromo-7-methyl-chromane (500 mg, 2.20 mmol) in TFA (5 mL) was added dropwise a solution of KNO₃ (244.86 mg, 2.42 mmol) in TFA (2 mL) at 0° C., then the mixture was stirred at 20° C. for 1 h. The reaction was added into water (15 mL) at 0° C., then extracted with ethyl acetate (20 mL*3). The combined organic layers were washed with saturated sodium bicarbonate solution (50 mL) and dried over anhydrous sodium sulfate, filtered and concentrated under reduced pressure to give a residue. The residue was purified by flash chromatography om silica gel to give 8-bromo-7-methyl-6-nitro-chromane (380 mg, crude) as a yellow oil.

¹H NMR (400 MHz, METHANOL-d₄) δ: 2.00-2.04 (m, 2H) 2.56 (s, 3H) 2.86 (t, J=6.38 Hz, 2H) 4.34-4.40 (m, 2H) 7.70 (s, 1H)

### Step 6. 8-bromo-7-methyl-chroman-6-amine

To a solution of 8-bromo-7-methyl-6-nitro-chromane (380 mg, 1.40 mmol) and NH₄Cl (747.04 mg, 13.97 mmol) in EtOH (4 mL) and H₂O (0.4 mL) was added Fe (389.96 mg, 6.98 mmol), then the mixture was stirred at 80° C. for 12 h. TLC (petroleum ether:ethyl acetate=5:1, Rf=0.2) indicated Reactant 1 remained, and three major new spots were detected. LCMS showed starting material remained and mass of the desired compound. The reaction was filtered and concentrated under reduced pressure, then added water (10 mL), then extracted with ethyl acetate (10 mL) and dried over anhydrous sodium sulfate, filtered and concentrated under reduced pressure to give a residue. The residue was purified by prep-TLC to give 8-bromo-7-methyl-chroman-6-amine (170 mg, 702.15 μmol, 50.28% yield) as a yellow oil.

¹H NMR (400 MHz, METHANOL-d₄) δ ppm 1.88-1.98 (m, 2H) 2.24 (s, 3H) 2.71 (t, J=6.56 Hz, 2H)4.11-4.19 (m, 2H)6.48 (s, 1H)

### Step 7. N2-(8-bromo-7-methyl-chroman-6-yl)-N4,6-dimethyl-pyrimidine-2,4-diamine

To a solution of 8-bromo-7-methyl-chroman-6-amine (50 mg, 206.52 mol) and 2-chloro-N,6-dimethyl-pyrimidin-4-amine (32.55 mg, 206.52 μmol) in i-PrOH (1.5 mL) was added TFA (2.35 mg, 20.65 μmol, 1.59 μL), then the mixture was stirred at 130° C. for 3 h. The reaction was concentrated under reduced pressure to give N2-(8-bromo-7-methyl-chroman-6-yl)-N4,6-dimethyl-pyrimidine-2,4-diamine (75 mg, crude) as a light-yellow solid.

### Step 8. tert-butyl 2-methyl-5-[7-methyl-6-[[4-methyl-6-(methylamino)pyrimidin-2-yl]amino]chroman-8-yl]-2,3,4,7-tetrahydroazepine-1-carboxylate

To a solution of N2-(8-bromo-7-methyl-chroman-6-yl)-N4,6-dimethyl-pyrimidine-2,4-diamine (75 mg, 206.47 μmol), tert-butyl 2-methyl-5-(4,4,5,5-tetramethyl-1,3,2-dioxaborolan-2-yl)-2,3,4,7-tetrahydroazepine-1-carboxylate (76.60 mg, 227.12 μmol), K₂CO₃ (85.61 mg, 619.41 mol) and H₂O (0.3 mL) in dioxane (3 mL) was added Pd(dppf)Cl₂ (15.11 mg, 20.65 μmol), then the mixture was stirred at 100° C. for 12 h under an atmosphere of nitrogen. The reaction was filtered and concentrated under reduced pressure to give tert-butyl2-methyl-5-[7-methyl-6-[[4-methyl-6-(methylamino)pyrimidin-2-yl]amino]chroman-8-yl]-2,3,4,7-tetrahydroazepine-1-carboxylate (100 mg, crude) as a brown solid.

### Step 9. N4,6-dimethyl-N2-[7-methyl-8-[rel-(2S)-2-methyl-2,3,4,7-tetrahydro-1H-azepin-5-yl]chroman-6-yl]pyrimidine-2,4-diamine,N4,6-dimethyl-N2-[7-methyl-8-[rel-(2R)-2-methyl-2,3,4,7-tetrahydro-1H-azepin-5-yl]chroman-6-yl]pyrimidine-2,4-diamine

To a solution of tert-butyl 2-methyl-5-[7-methyl-6-[[4-methyl-6-methylamino)pyrimidin-2-yl]amino]chroman-8-yl]-2,3,4,7-tetrahydroazepine-1-carboxylate (100 mg, 202.58 μmol) in DCM (3 mL) was added ZnBr₂ (228.10 mg, 1.01 mmol), then the mixture was stirred at 35° C. for 12 h. The reaction was concentrated under reduced pressure to give a residue. The residue was purified by prep-HPLC (TFA condition, column: Phenomenex Luna 80*30 mm*3 um; mobile phase: [water(TFA)-ACN; B %: 1%-30%, 8 min) to give crude product. The crude product was purified by Chiral-SFC (Column: DAICEL CHIRALPAK IG(250 mm*30 mm,10 um); Mobile phase: A for Heptane and B for EtOH(0.1% IPA); Gradient: B %=40-40%; Flow rate: 50 ml/min) to give N4,6-dimethyl-N2-[7-methyl-8-[rel-(2S)-2-methyl-2,3,4,7-tetrahydro-1H-azepin-5-yl]chroman-6-yl]pyrimidine-2,4-diamine (8.4 mg, 21.35 μmol, 10.54% yield) (purity: 99.586%, ee=100%) as a light-yellow solid and N4,6-dimethyl-N2-[7-methyl-8-[rel-(2R)-2-methyl-2,3,4,7-tetrahydro-1H-azepin-5-yl]chroman-6-yl]pyrimidine-2,4-diamine (8.6 mg, 21.85 μmol, 10.79% yield) (purity: 99.255, ee=97.28%) as a yellow solid.

¹H NMR (400 MHz, METHANOL-d₄) δ ppm 1.19 (t, J=5.94 Hz, 3H) 1.51-1.83 (m, 1H) 1.85-2.01 (m, 3H) 2.03-2.20 (m, 6H) 2.23-2.34 (m, 1H) 2.44-2.66 (m, 1H) 2.75 (br s, 2H) 2.81 (s, 3H) 3.04-3.15 (m, 1H) 3.39-3.57 (m, 2H) 4.05-4.19 (m, 2H) 5.49-5.63 (m, 1H) 5.73 (s, 1H) 7.14 (br s, 1H)

¹H NMR (400 MHz, METHANOL-d₄) δ:1.18 (dd, J=6.50, 3.38 Hz, 3H) 1.51-1.80 (m, 1H) 1.84-2.01 (m, 3H) 2.04-2.20 (m, 6H) 2.21-2.36 (m, 1H) 2.42-2.65 (m, 1H) 2.71-2.78 (m, 2H) 2.81 (d, J=0.88 Hz, 3H) 2.98-3.13 (m, 1H) 3.36-3.54 (m, 2H) 4.05-4.20 (m, 2H) 5.49-5.62 (m, 1H) 5.73 (s, 1H) 7.14 (br s, 1H)

### EXAMPLE 83. Synthesis of Compound 422, Compound 420, Compound 401 and Compound 398

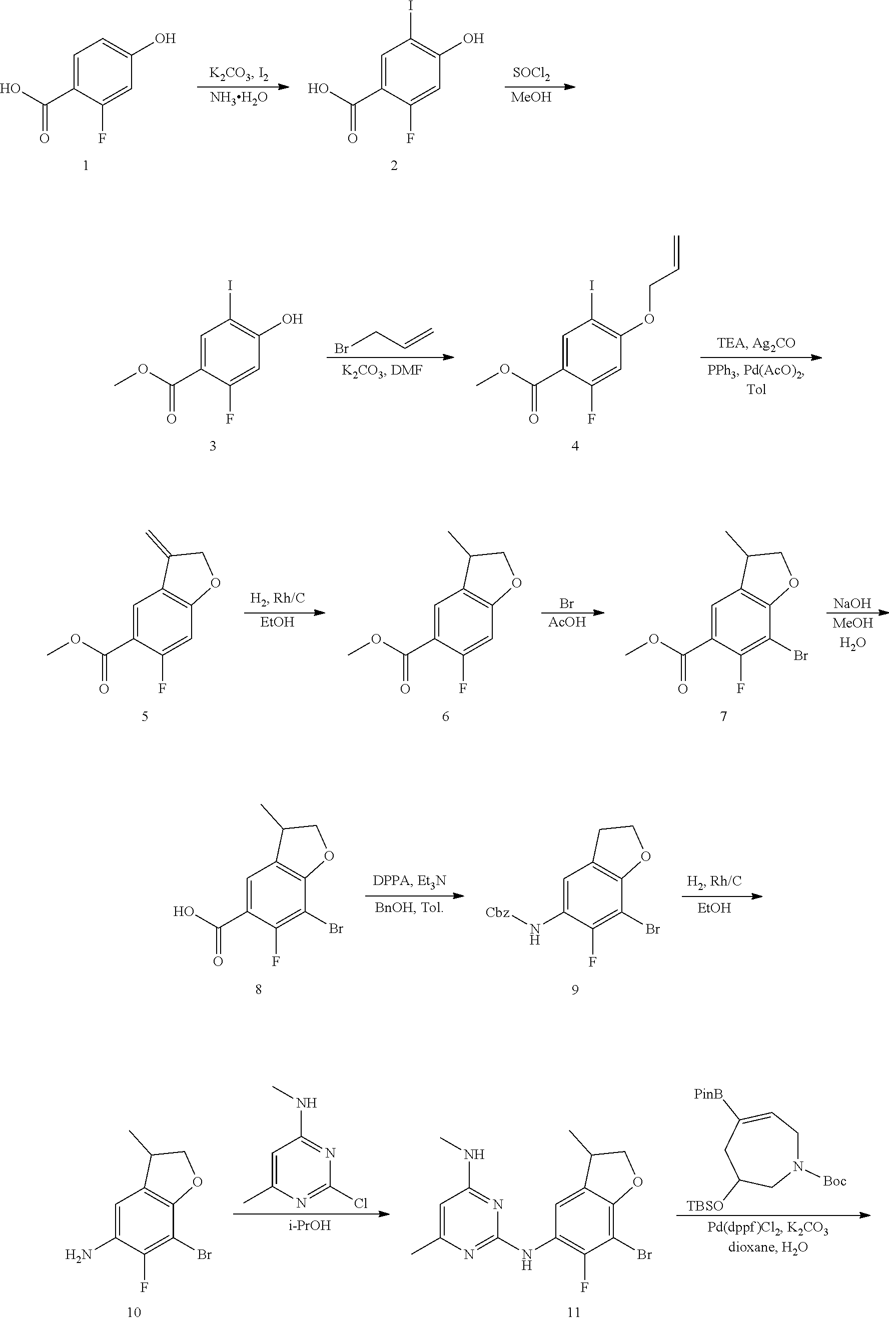

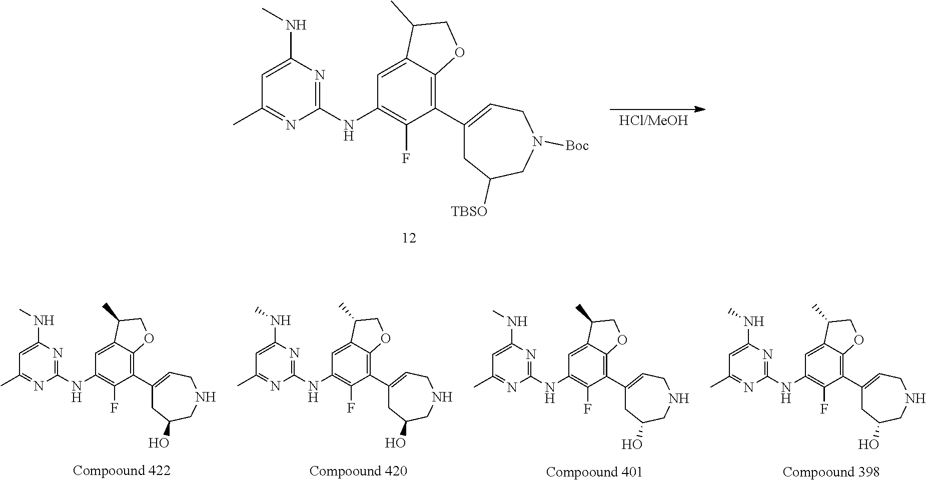

### Step 1. 2-fluoro-4-hydroxy-5-iodo-benzoic acid

K₂CO₃ (66.40 g, 480.43 mmol) was dissolved in NH₃·H₂O (500 mL), 12 (40.65 g, 160.14 mmol) was added, then the mixture was stirred at 25° C. for 1 hr. 2-fluoro-4-hydroxy-benzoic acid (25 g, 160.14 mmol) was added to the mixture was stirred at 25° C. for 2 hrs. The reaction mixture was acidified by HCl (2 M) till pH=1. The reaction mixture was added to water (200 mL), extracted with EtOAc (500 mL*3). The combined organic layers were washed with 500 mL of brine, dried over Na₂SO₄, filtered and concentrated under reduced pressure. 2-fluoro-4-hydroxy-5-iodo-benzoic acid (35 g, crude) was obtained as a yellow solid.

### Step 2. methyl 2-fluoro-4-hydroxy-5-iodo-benzoate

To a mixture of 2-fluoro-4-hydroxy-5-iodo-benzoic acid (35 g, 124.11 mmol) in MeOH (500 mL) was added SOCl₂ (30 mL) at 0° C., then the mixture was stirred at 70° C. for 2 hrs. The mixture was concentrated in vacuum to give a residue. methyl 2-fluoro-4-hydroxy-5-iodo-benzoate (36 g, crude) was obtained as a white solid.

### Step 3. methyl 4-allyloxy-2-fluoro-5-iodo-benzoate

methyl 2-fluoro-4-hydroxy-5-iodo-benzoate (36 g, 121.61 mmol) was dissolved in DMF (360 mL), and then to the mixture was added K₂CO₃ (33.61 g, 243.22 mmol) and 3-bromoprop-1-ene (16.18 g, 133.77 mmol), and then the mixture was stirred at 50° C. for 12 hrs. The mixture was concentrated in vacuum. The residue was purified by column chromatography of silica gel to give methyl 4-allyloxy-2-fluoro-5-iodo-benzoate (29 g, 86.28 mmol, 70.95% yield) as a white solid.

### Step 4. methyl 6-fluoro-3-methylene-benzofuran-5-carboxylate

To a solution of methyl 4-allyloxy-2-fluoro-5-iodo-benzoate (5 g, 14.88 mmol), TEA (3.01 g, 29.75 mmol, 4.15 mL), Ag₂CO₃ (8.20 g, 29.75 mmol) and PPh₃ (780.38 mg, 2.98 mmol) in Tol. (100 mL) was added Pd(AcO)₂ (333.99 mg, 1.49 mmol), then the mixture was stirred at 70° C. for 12 h under N2. The reaction was concentrated under reduced pressure to give a residue. The residue was purified by column chromatography of silica gel to give methyl 6-fluoro-3-methylene-benzofuran-5-carboxylate (2 g, crude) as a white solid.

### Step 5. methyl 6-fluoro-3-methyl-2,3-dihydrobenzofuran-5-carboxylate

To a mixture of methyl 6-fluoro-3-methylene-benzofuran-5-carboxylate (6 g, 28.82 mmol) in MeOH (400 mL) was added Pd/C (3.00 g) under H₂, then the mixture was stirred at 60° C. for 12 hr under H₂ (15 psi). The reaction was filtered through a pad of celite and concentrated under reduced pressure to give a residue. methyl 6-fluoro-3-methyl-2,3-dihydrobenzofuran-5-carboxylate (4.5 g, crude) was obtained as a yellow solid

### Step 6. methyl 7-bromo-6-fluoro-3-methyl-2,3-dihydrobenzofuran-5-carboxylate

To a mixture of methyl 6-fluoro-3-methyl-2,3-dihydrobenzofuran-5-carboxylate (9 g, 42.82 mmol) in AcOH (70 mL) was added Br₂ (20.53 g, 128.45 mmol, 6.58 mL) at 0° C., then the mixture was stirred at 25° C. for 12 hrs. The reaction was concentrated under reduced pressure to give a residue. The residue was purified by flash column chromatography on silica gel to give methyl 7-bromo-6-fluoro-3-methyl-2,3-dihydrobenzofuran-5-carboxylate (12 g, crude) as a yellow solid.

### Step 7. 7-bromo-6-fluoro-3-methyl-2,3-dihydrobenzofuran-5-carboxylic acid

To a solution of methyl 7-bromo-6-fluoro-3-methyl-2,3-dihydrobenzofuran-5-carboxylate (7.7 g, 26.63 mmol) in MeOH (60 mL) and H₂O (20 mL) was added NaOH (2.13 g, 53.27 mmol, 1.00 mL). It was stirred at 20° C. for 12 hrs. The reaction was concentrated under reduced pressure to give a residue. The residue was acidified by HCl (2 M) till pH=1. The reaction mixture was filtered to get a cake, The cake was the product. The filtrate was added to water (100 ml), extracted with EtOAc (100 ml*3). The combined organic layers was dried over Na₂SO₄, filtered and concentrated under reduced pressure to 7-bromo-6-fluoro-3-methyl-2,3-dihydrobenzofuran-5-carboxylic acid (6 g, crude) as a yellow solid.

### Step 8. benzyl N-(7-bromo-6-fluoro-3-methyl-2,3-dihydrobenzofuran-5-yl)carbamate

To a solution of 7-bromo-6-fluoro-3-methyl-2,3-dihydrobenzofuran-5-carboxylic acid (5.7 g, 20.72 mmol) in Tol. (50 mL) was added DPPA (6.05 g, 24.87 mmol), TEA (6.29 g, 62.17 mmol, 8.66 mL). It was stirred at 20° C. for 1 h under N2. Then it was added BnOH (2.79 g, 62.17 mmol). It was stirred at 80° C. for 12 hrs under N2. The reaction was concentrated under reduced pressure to give benzyl N-(7-bromo-6-fluoro-3-methyl-2,3-dihydrobenzofuran-5-yl)carbamate (10 g, crude) as a brown soild.

### Step 9. 7-bromo-6-fluoro-3-methyl-2,3-dihydrobenzofuran-5-amine

benzyl N-(7-bromo-6-fluoro-3-methyl-2,3-dihydrobenzofuran-5-yl)carbamate (7.6 g, 19.99 mmol) in EtOH (160 mL) was added Rh/C (3 g), then the mixture was stirred at 25° C. for 5 hrs under H₂ (19.99 mmol) (15 psi). The reaction mixture was concentrated to give a residue. The residue was purified by column chromatography of silica gel to give 7-bromo-6-fluoro-3-methyl-2,3-dihydrobenzofuran-5-amine (500 mg, crude) as yellow oil and benzyl N-(7-bromo-6-fluoro-3-methyl-2,3-dihydrobenzofuran-5-yl)carbamate (3.7 g, crude) as a yellow soild.

### Step 10. N2-(7-bromo-6-fluoro-3-methyl-2,3-dihydrobenzofuran-5-yl)-N4,6-dimethyl-pyrimidine-2,4-diamine

To the mixture of 7-bromo-6-fluoro-3-methyl-2,3-dihydrobenzofuran-5-amine (450 mg, 1.83 mmol), 2-chloro-N,6-dimethyl-pyrimidin-4-amine (345.85 mg, 2.19 mmol) in i-PrOH (5 mL) and TFA (0.01 mL), then stirred at 130° C. for 2 hrs. The reaction was concentrated to give N2-(7-bromo-6-fluoro-3-methyl-2,3-dihydrobenzofuran-5-yl)-N4,6-dimethyl-pyrimidine-2,4-diamine (200 mg, crude) as brown solid.

### Step 11. tert-butyl 3-[tert-butyl(dimethyl)silyl]oxy-5-[6-fluoro-3-methyl-5-[[4-methyl-6 (methylamino) pyrimidin-2-yl]amino]-2,3-dihydrobenzofuran-7-yl]-2,3,4,7-tetrahydroazepine-1-carboxylate

To a mixture of N2-(7-bromo-6-fluoro-3-methyl-2,3-dihydrobenzofuran-5-yl)-N4,6-dimethyl-pyrimidine-2,4-diamine (270 mg, 735.26 μmol) and tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (333.44 mg, 735.26 μmol) in dioxane (10 mL) and H₂O (1 mL) was added K₂CO₃ (508.09 mg, 3.68 mmol) and cyclopentyl(diphenyl)phosphane;dichloromethane;dichloropalladium;iron (60.04 mg, 73.53 mol) under N2. The reaction was stirred at 100° C. for 12 hrs under N2. The mixture was filtered and concentrated under reduced pressure to give a residue tert-butyl 3-[tert-butyl(dimethyl)silyl]oxy-5-[6-fluoro-3-methyl-5-[[4-methyl-6(methylamino) pyrimidin-2-yl]amino]-2,3-dihydrobenzofuran-7-yl]-2,3,4,7-tetrahydroazepine-1-carboxylate (400 mg, crude) was obtained as brown oil.

### Step 12. rel-(3S)-5-[rel-(3R)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol, rel-(3S)-5-[rel-(3S)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol, rel-(3R)-5-[rel-(3R)-6-fluoro-3-methyl-5-[[4-methyl-6-(methylamino)pyrimidin-2-yl]amino]-2,3-dihydrobenzofuran-7-yl-2,3,4,7-tetrahydro-1H-azepin-3-ol and rel-(3R)-5-[rel-(3S)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol

The solution of tert-butyl 3-[tert-butyl(dimethyl)silyl]oxy-5-[6-fluoro-3-methyl-5-[[4-methyl-6-(methylamino)pyrimidin-2-yl]amino]-2,3-dihydrobenzofuran-7-yl]-2,3,4,7-tetrahydroazepine-1-carboxylate (50 mg, 81.45 μmol) in HCl/MeOH (5 mL) was stirred at 25° C. for 1 hr. The reaction was filtered and concentrated to give a residue. The residue was purified by prep-HPLC (TFA condition; column: 3_Phenomenex Luna C18 75*30 mm*3 um; mobile phase:water(0.2% TFA)-ACN; B %: 1%-30%,8 min) and then separated by SFC (Column: DAICEL CHIRALPAK IH(250 mm*30 mm,10 um); Mobile phase: Heptane-EtOH(0.1% IPA)); Acq Method: AD10_H_E_lb_50_10_MS) to give 10.9 mg P1 (Rt=2.526 min),10.3 mg P2 (Rt=2.742 min),10.9 mg P3(Rt=3.531 min) and 10.2 mg P4(Rt=6.220 min).

rel-(3S)-5-[rel-(3R)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol, (50 mg, 81.45 μmol) was obtained as a pale yellow solid.

¹H NMR (400 MHz, METHANOL-d₄) δ: 7.95-7.83 (m, 1H), 6.10 (t, J=6.0 Hz, 1H), 5.83-5.77 (m, 1H), 4.74-4.68 (m, 1H), 4.07 (t, J=8.1 Hz, 1H), 3.95-3.87 (m, 1H), 3.57-3.33 (m, 4H), 2.93-2.84 (m, 5H), 2.72-2.65 (m, 1H), 2.16 (s, 3H), 1.33-1.29 (m, 3H)
rel-(3S)-5-[rel-(3S)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol (10.3 mg, 25.78 μmol, 31.66% yield) was obtained as a pale yellow solid.

¹H NMR (400 MHz, METHANOL-d₄) δ: 7.99-7.80 (m, 1H), 6.12-6.06 (m, 1H), 5.80 (s, 1H), 4.72-4.66 (m, 1H), 4.11-4.04 (m, 1H), 3.93-3.85 (m, 1H), 3.56-3.32 (m, 4H), 2.92-2.83 (m, 5H), 2.70-2.63 (m, 1H), 2.19-2.12 (m, 3H), 1.33-1.29 (m, 3H)
rel-(3R)-5-[rel-(3R)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol (10.9 mg, 27.29 μmol, 33.50% yield) was obtained as white solid.

¹H NMR (400 MHz, METHANOL-d₄) δ:7.95-7.82 (m, 1H), 6.14-6.04 (m, 1H), 5.82-5.74 (m, 1H), 4.77-4.63 (m, 1H), 4.06 (t, J=8.1 Hz, 1H), 3.94-3.85 (m, 1H), 3.56-3.32 (m, 4H), 2.92-2.83 (m, 5H), 2.68 (br d, J=14.9 Hz, 1H), 2.16 (s, 3H), 1.31 (d, J=6.8 Hz, 3H)
rel-(3R)-5-[rel-(3S)-6-fluoro-3-methyl-5-[[4-methyl-6-(methylamino)pyrimidin-2-yl]amino]-2,3-dihydrobenzofuran-7-yl]-2,3,4,7-tetrahydro-1H-azepin-3-ol (10.2 mg, 25.53 μmol, 31.35% yield) was obtained.

¹H NMR (400 MHz, METHANOL-d₄) δ: 7.97-7.81 (m, 1H), 6.13-6.06 (m, 1H), 5.80 (s, 1H), 4.69 (t, J=8.8 Hz, 1H), 4.13-4.05 (m, 1H), 3.99-3.89 (m, 1H), 3.59-3.45 (m, 3H), 3.37 (dd, J=2.9, 13.1 Hz, 1H), 2.99-2.85 (m, 5H), 2.75-2.67 (m, 1H), 2.19-2.13 (m, 3H), 1.33-1.29 (m, 3H)

### EXAMPLE 84. Synthesis of Compound 408, Compound 407, Compound 403 and Compound 402

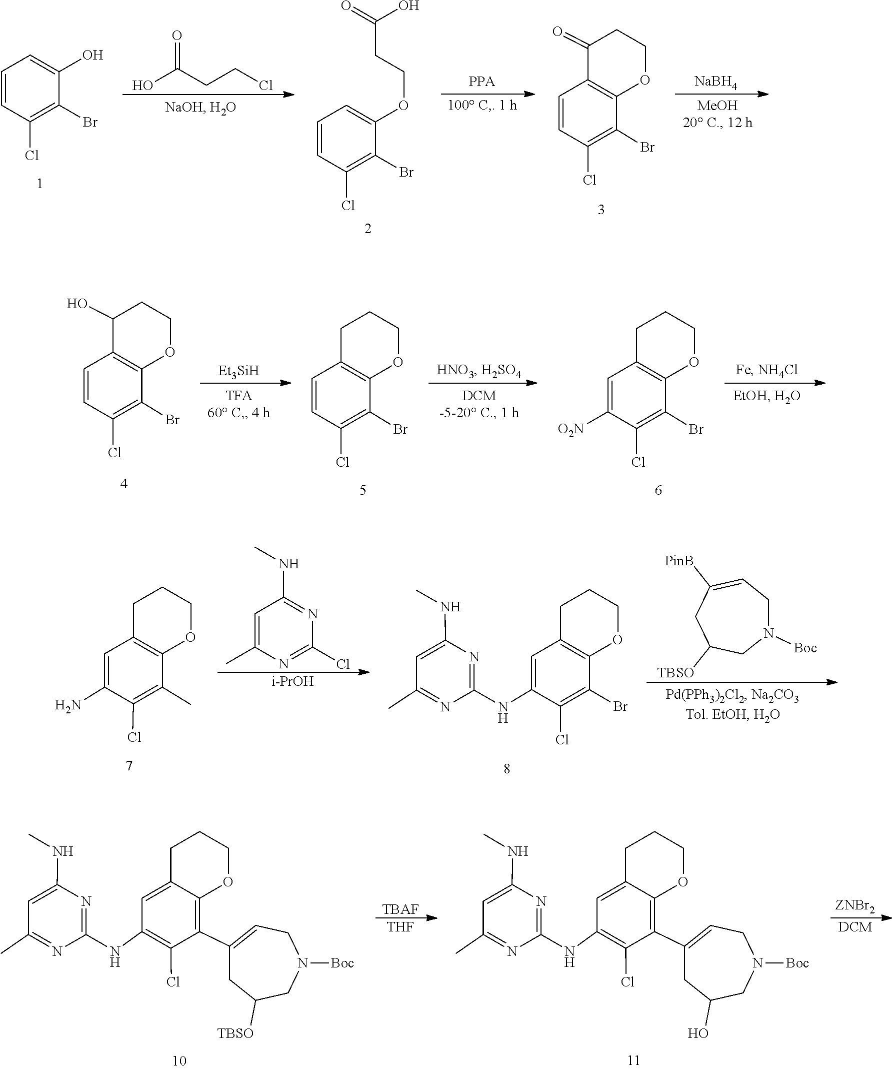

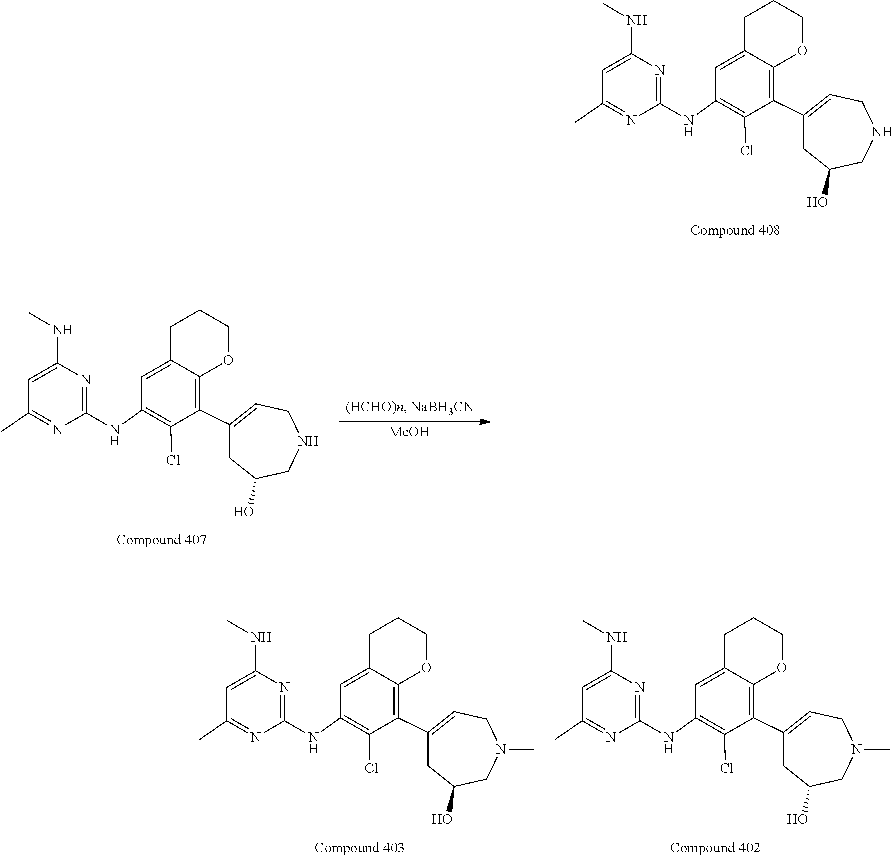

### Step 1. 3-(2-bromo-3-fluoro-phenoxy)propanoic

A mixture of 2-bromo-3-fluoro-phenol (2.8 g, 14.66 mmol) and 3-chloropropanoic acid (1.91 g, 17.59 mmol, 1.50 mL), then was added dropwise a solution of NaOH (1.41 g, 35.18 mmol, 660.68 μL) in H₂O (30.56 mL) at 20° C., then the mixture was stirred at 100° C. for 12 hrs. The 5 batches were combined to work up. The reaction was added aq. HCl(1N) to pH=4, then extracted with ethyl acetate (3*100 mL) and dried over anhydrous sodium sulfate, filtered and concentrated under reduced pressure to give a residue. The residue was purified by column chromatography on silica gel. 3-(2-bromo-3-fluoro-phenoxy)propanoic acid (8.5 g, crude) was obtained as a white solid.

### Step 2. 8-bromo-7-fluoro-chroman-4-one

A solution of 3-(2-bromo-3-fluoro-phenoxy)propanoic acid (2.13 g, 8.08 mmol) in PPA (60 mL), then the mixture was stirred at 100° C. for 2 h. The four reactions were work up together. The mixture was concentrated in vacuum to remove PPA and then dissolved in water (200 mL), extracted with EtOAc (100 mL*3) and dried over anhydrous sodium sulfate, filtered and concentrated under reduced pressure to give 8-bromo-7-fluoro-chroman-4-one (6 g, crude) as a yellow solid.

### Step 3. 8-bromo-7-chloro-chroman-4-ol

To a solution of 8-bromo-7-chloro-chroman-4-one (2 g, 7.65 mmol) in MeOH (50 mL) was added NaBH₄ (434.02 mg, 11.47 mmol) at 0° C., then the mixture was stirred at 20° C. for 1 h.

The reaction was concentrated under reduced pressure. The residue was purified by column chromatography on silica gel. 8-bromo-7-chloro-chroman-4-ol (2 g, crude) was obtained as yellow oil.

¹H NMR (400 MHz, CHLOROFORM-d) δ: 7.19 (d, J=8.2 Hz, 1H), 7.02 (d, J=8.2 Hz, 1H), 4.75 (t, J=4.2 Hz, 1H), 4.45-4.30 (m, 2H), 2.05-2.00 (m, 2H)

### Step 4. 8-bromo-7-chloro-chromane

To a solution of 8-bromo-7-chloro-chroman-4-ol (2 g, 7.59 mmol) in TFA (20 mL) was added Et3SiH (1.77 g, 15.18 mmol, 2.42 mL), then the mixture was stirred at 60° C. for 4 h. The reaction was added into water (10 mL), then extracted with ethyl acetate (3*15 mL). The combined organic layers were washed with brine (10 mL) and dried over anhydrous sodium sulfate, filtered and concentrated under reduced pressure to give a residue. The residue was purified by column chromatography on silica gel. 8-bromo-7-chloro-chromane (2 g, crude) was obtained as clear oil.

¹H NMR (400 MHz, CHLOROFORM-d) δ: 6.97-6.90 (m, 2H), 4.34-4.29 (m, 2H), 2.82-2.73 (m, 2H), 2.04-1.98 (m, 2H)

### Step 5. 8-bromo-7-chloro-6-nitro-chromane

To a solution of 8-bromo-7-chloro-chromane (2.2 g, 8.89 mmol) in DCM (50 mL) was added a mixture of nitric acid (588.08 mg, 9.33 mmol) and H₂SO₄ (12 M, 2.96 mL) at 0° C., then the mixture was stirred at 20° C. for 1 h. The reaction was added into water (25 mL) at 0° C., then extracted with dichloromethane (50 mL*3). The combined organic layers were washed with saturated sodium bicarbonate solution (100 mL*3) and dried over anhydrous sodium sulfate, filtered and concentrated under reduced pressure. The residue was purified by column chromatography on silica gel. 8-bromo-7-chloro-6-nitro-chromane (1 g, crude) was obtained as brown solid.

### Step 6. 8-bromo-7-chloro-chroman-6-amine

To a solution of 8-bromo-7-chloro-6-nitro-chromane (1 g, 3.42 mmol), NH₄Cl (182.87 mg, 3.42 mmol) and EtOH (20 mL) in H₂O (2 mL) was added Fe (190.91 mg, 3.42 mmol), then the mixture was stirred at 80° C. for 12 hrs. The reaction was filtered and concentrated under reduced pressure to give a residue. The reaction mixture was added to water (20 mL), extracted with EtOAc (50 mL*5). The organic layer was dried over Na₂SO₄, concentrated to give 8-bromo-7-chloro-chroman-6-amine (450 mg, crude) as brown oil.

### Step 7. N2-(8-bromo-7-chloro-chroman-6-yl)-N4,6-dimethyl-pyrimidine-2,4-diamine

A mixture of 2-chloro-N,6-dimethyl-pyrimidin-4-amine (162.08 mg, 1.03 mmol) and 8-bromo-7-chloro-chroman-6-amine (225 mg, 857.04 μmol) in i-PrOH (3 mL) was stirred at 130° C. for 2 hrs. The reaction was concentrated under reduced pressure to give N2-(8-bromo-7-chloro-chroman-6-yl)-N4,6-dimethyl-pyrimidine-2,4-diamine (430 mg, crude) as white soild.

### Step 8. Tert-butyl 3-[tert-butyl(dimethyl)silyl]oxy-5-[7-chloro-6-[[4-methyl-6-(methylamino)pyrimidin-2-ylamino]chroman-8-yl]-1-2,3,4,7-tetrahydroazepine-1-carboxylate

To a mixture of tert-butyl 3-[tert-butyl(dimethyl)silyl]oxy-5-(4,4,5,5-tetramethyl-1,3,2-dioxaborolan-2-yl)-2,3,4,7-tetrahydroazepine-1-carboxylate (236.40 mg, 521.28 μmol), N2-(8-bromo-7-chloro-chroman-6-yl)-N4,6-dimethyl-pyrimidine-2,4-diamine (200 mg, 521.28 μmol) in Tol. (7 mL), H₂O (1 mL) and EtOH (3 mL) was added Na₂CO₃ (165.75 mg, 1.56 mmol) and Pd(PPh₃)₂Cl₂ (36.59 mg, 52.13 μmol) under N₂ atmosphere. The mixture was stirred at 100° C. for 12 h under N₂ atmosphere. The reaction was concentrated under reduced pressure to give Tert-butyl 3-[tert-butyl(dimethyl)silyl]oxy-5-[7-chloro-6-[[4-methyl-6-(methylamino)pyrimidin-2-yl]amino]chroman-8-yl]-2,3,4,7-tetrahydroazepine-1-carboxylate (300 mg, crude) was obtained as brown oil.

### Step 9. rel-(3S)-5-[7-chloro-6-[[4-methyl-6-(methylamino)pyrimidin-2-yl]amino]chroman-8-yl-2,3,4,7-tetrahydro-1H-azepin-3-ol and rel-(3R)-5-[7-chloro-6-[[4-methyl-6-(methylamino)pyrimidin-2-ylamino]chroman-8-yl]-1-2,3,4,7-tetrahydro-1H-azepin-3-ol

A solution of tert-butyl 3-[tert-butyl(dimethyl)silyl]oxy-5-[7-chloro-6-[[4-methyl-6-(methylamino)pyrimidin-2-yl]amino]chroman-8-yl]-2,3,4,7-tetrahydroazepine-1-carboxylate (200 mg, 317.31 μmol) in HCl/MeOH (3 mL) was stirred at 25° C. for 1 hrs. The mixture was concentrated under reduced pressure at 25° C. The residue was separated by SFC (column: DAICEL CHIRALCEL OX (250 mm*30 mm, 10 um); mobile phase: 0.1% NH₃H₂O IPA; B %: 40%-40%; SFC Method: IG_H_E_ACN_IPAm_30_1_30_10 CM). Rel-(3S)-5-[7-chloro-6-[[4-methyl-6-(methylamino)pyrimidin-2-yl]amino]chroman-8-yl]-2,3,4,7-tetrahydro-1H-azepin-3-ol (12.0 mg, 28.85 μmol, 9.09% yield) (ee %=99.68%; Rt=4.631) was obtained as a yellow solid. Rel-(3R)-5-[7-chloro-6-[[4-methyl-6-(methylamino)pyrimidin-2-yl]amino]chroman-8-yl]-2,3,4,7-tetrahydro-1H-azepin-3-ol (10.6 mg, 25.49 μmol, 8.03% yield) (ee %=96.68%; Rt=5.639) was obtained as a pale yellow solid.

¹H NMR (400 MHz, METHANOL-d₄) δ: 7.90 (br d, J=9.78 Hz, 1H) 5.88 (s, 1H) 5.79 (br t, J=5.75 Hz, 1H) 4.16-4.23 (m, 3H) 3.80-3.89 (m, 2H) 3.50-3.59 (m, 1H) 3.15-3.29 (m, 1H) 2.87-2.93 (m, 4H) 2.82 (br t, J=6.36 Hz, 2H) 2.58-2.77 (m, 1H) 2.20 (s, 3H) 2.00 (br d, J=5.62 Hz, 2H)

¹H NMR (400 MHz, METHANOL-d₄) δ: 7.96-8.09 (m, 1H) 5.82 (s, 1H) 5.77 (br t, J=5.69 Hz, 1H)4.13-4.19 (m, 2H)3.97-4.04 (m, 1H)3.44-3.52 (m, 2H)2.83-2.98 (m, 5H) 2.78-2.83 (m, 3H) 2.40-2.58 (m, 1H) 2.15-2.19 (m, 3H) 1.96-2.01 (m, 2H)

### Step 10. rel-(3S)-5-[7-chloro-6-[[4-methyl-6-(methylamino)pyrimidin-2-yl]amino]chroman-8-yl]-1-methyl-2,3,4,7-tetrahydroazepin-3-ol and rel-(3R)-5-[7-chloro-6-[{4-methyl-6-(methylamino)pyrimidin-2-ylamino]chroman-8-yl]-1-methyl-2,3,4,7-tetrahydroazepin-3-ol

To a solution of 5-[7-chloro-6-[[4-methyl-6-(methylamino)pyrimidin-2-yl]amino]chroman-8-yl]-2,3,4,7-tetrahydro-1H-azepin-3-ol (20 mg, 48.09 μmol) in MeOH (2 mL) was added formaldehyde (1.44 mg, 48.09 μmol, 1.33 μL), then the mixture was added sodium;cyanoboranuide (9.07 mg, 144.26 μmol) and stirred at 25° C. for 12 h. The reaction was filtered and concentrated under reduced pressure to give a residue. The crude product was purified by prep-HPLC (TFA condition:column: Phenomenex Luna 80*30 mm*3 um; mobile phase:water(TFA)-ACN; B %: 10%-30%, 8 min) to give product. The residue was separated by SFC (column: DAICEL CHIRALCEL OX (250 mm*30 mm,10 um); mobile phase: 0.1% NH₃H₂O IPA; B %: 20%-20%;), Method: OD10_H_E_lb_20_10_MS). Rel-(3S)-5-[7-chloro-6-[[4-methyl-6-(methylamino)pyrimidin-2-yl]amino]chroman-8-yl]-1-methyl-2,3,4,7-tetrahydroazepin-3-ol (7.4 mg, 17.21 μmol, 35.79% yield) (ee %=100%; Rt=2.065) was obtained as a pale yellow solid. Rel-(3R)-5-[7-chloro-6-[[4-methyl-6-(methylamino)pyrimidin-2-yl]amino]chroman-8-yl]-1-methyl-2,3,4,7-tetrahydroazepin-3-ol (7.2 mg, 16.75 μmol, 34.83% yield) (ee %=100^{%}; Rt=2.727) was obtained as a pale yellow solid.

¹H NMR (400 MHz, METHANOL-d₄) δ: 7.91 (br s, 1H) 5.86 (s, 1H) 5.73 (br d, J=4.28 Hz, 1H) 4.12-4.22 (m, 3H) 3.48-3.66 (m, 2H) 3.36 (brd, J=14.31 Hz, 1H) 2.92-3.06 (m, 1H) 2.90 (s, 3H) 2.78-2.87 (m, 3H) 2.68 (d, J=4.89 Hz, 3H) 2.42-2.61 (m, 1H) 2.19 (s, 3H) 2.00 (dq, J=10.90, 5.54 Hz, 2H)

¹H NMR (400 MHz, METHANOL-d₄) δ: 7.87 (br s, 1H) 5.87 (s, 1H) 5.74 (br d, J=3.18 Hz, 1H) 4.14-4.22 (m, 3H) 3.56-3.73 (m, 2H) 3.40 (br d, J=14.92 Hz, 1H)2.98-3.10 (m, 1H) 2.90 (s, 3H) 2.79-2.87 (m, 3H) 2.73 (d, J=3.55 Hz, 3H) 2.46-2.64 (m, 1H) 2.20 (s, 3H) 1.96-2.03 (m, 2H).

### EXAMPLE 85. Synthesis of Compound 415, Compound 414, Compound 409 and Compound 406

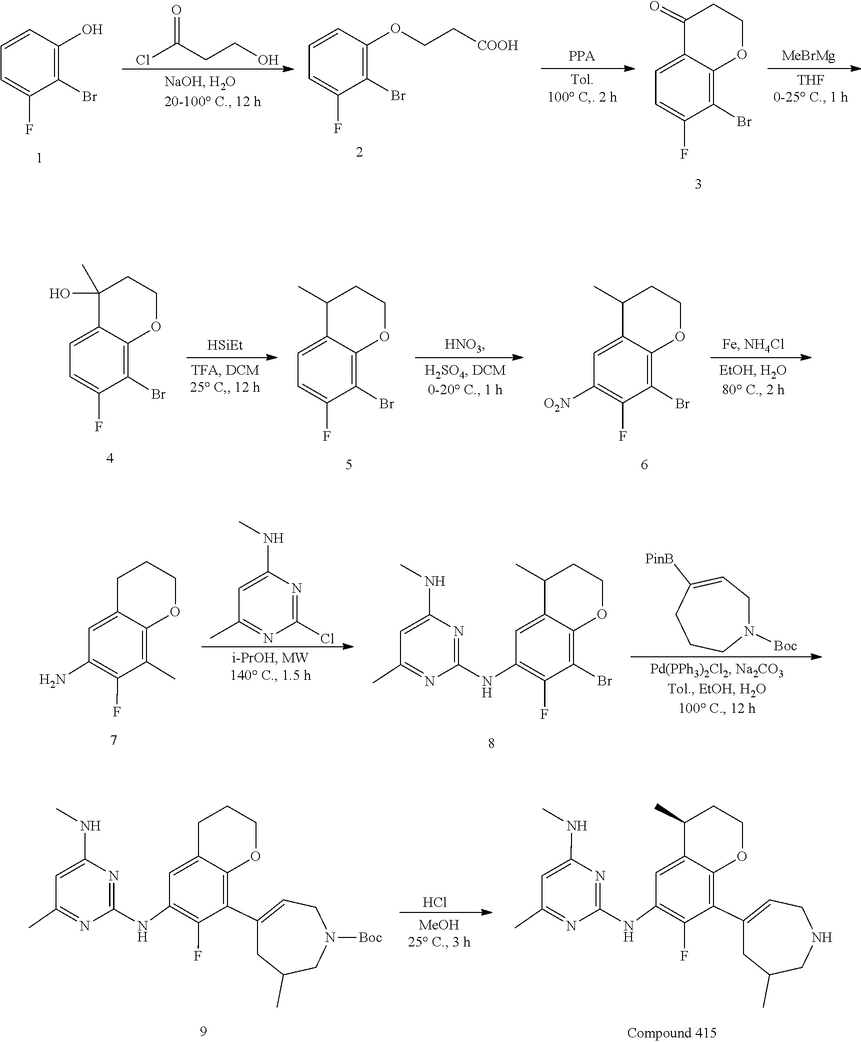

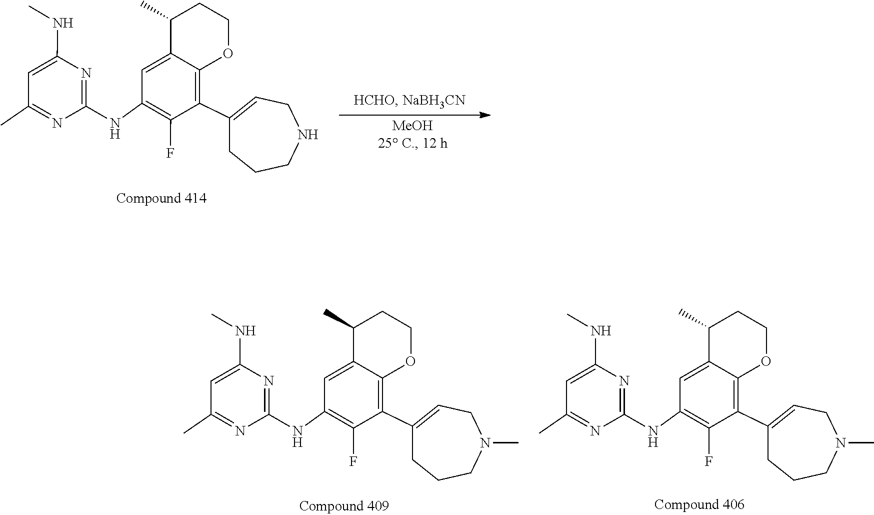

### Step 1. 3-(2-bromo-3-fluoro-phenoxy)propanoic acid

A mixture of 2-bromo-3-fluoro-phenol (5 g, 26.18 mmol) and 3-chloropropanoic acid (3.41 g, 31.41 mmol, 2.68 mL), then was added dropwise a solution of NaOH (2.51 g, 62.83 mmol, 1.18 mL) in H₂O (50 mL) at 20° C., then the mixture was stirred at 100° C. for 12 hrs. The reaction was added 1N HCl to pH=4, then extracted with ethyl acetate (100 mL*3) and dried over anhydrous sodium sulfate, filtered and concentrated under reduced pressure to give a residue. The residue was purified by column chromatography on silica gel to give 3-(2-bromo-3-fluoro-phenoxy)propanoic acid (20 g, crude) as a white solid.

### Step 2. 8-bromo-7-fluoro-chroman-4-one

A solution of 3-(2-bromo-3-fluoro-phenoxy)propanoic acid (10 g, 38.01 mmol) in PPA (40 ml), then the mixture was stirred at 100° C. for 2 h. The reaction mixture was quenched by water (200 mL) and extracted with EtOAc (200 mL*3) and dried over anhydrous sodium sulfate, filtered and concentrated under reduced pressure to give 8-bromo-7-fluoro-chroman-4-one (9 g, crude) as a yellow solid.

### Step 3. 8-bromo-7-fluoro-4-methyl-chroman-4-ol

To a solution of 8-bromo-7-fluoro-chroman-4-one (10 g, 40.81 mmol) in THF (150 mL) was added methylmagnesium(1+);bromide (3 M, 40.81 mL) at 0° C. under N2, then the mixture was stirred at 25° C. for 1 h under N2. The reaction mixture was quenched by ice water (500 mL), extracted with EtOAc (100 mL*3). The organic layer was dried over Na₂SO₄, concentrated to give the crude product. The residue was purified by column chromatography on silica gel. 8-bromo-7-fluoro-4-methyl-chroman-4-ol (3 g, crude) was obtained as a yellow oil.

### Step 4. 8-bromo-7-fluoro-4-methyl-chromane

To a solution of 8-bromo-7-fluoro-4-methyl-chroman-4-ol (3 g, 11.49 mmol) in DCM (30 mL) was added triethylsilane (2.00 g, 17.24 mmol, 2.75 mL) and TFA (13.10 g, 114.90 mmol, 8.79 mL), then the mixture was stirred at 25° C. for 12 h. The reaction solution was alkalined by sat·NaHCO₃ till pH=8, extracted with EtOAc (20 mL*3). The organic layer was dried over Na₂SO₄, concentrated to give the crude product. The residue was purified by column chromatography on silica gel. 8-bromo-7-fluoro-4-methyl-chromane (500 mg, crude) was obtained as a colourless oil.

### Step 5. 8-bromo-7-fluoro-4-methyl-6-nitro-chromane

To a solution of 8-bromo-7-fluoro-4-methyl-chromane (400 mg, 1.63 mmol) in DCM (8 mL) was added a mixture of nitric acid (113.13 mg, 1.80 mmol) and H₂SO₄ (12 M, 544.02 μL) at 0° C., then the mixture was stirred at 20° C. for 1 h. The reaction mixture was quenched by ice (20 mL), extracted with EtOAc (10 mL*3). The organic layer was dried over Na₂SO₄, concentrated to give the crude product. The residue was purified by prep-TLC. 8-bromo-7-fluoro-4-methyl-6-nitro-chromane (300 mg, crude) was obtained as a yellow solid.

### Step 6. 8-bromo-7-fluoro-4-methyl-chroman-6-amine

To a solution of 8-bromo-7-fluoro-4-methyl-6-nitro-chromane (300 mg, 1.03 mmol), NH₄Cl (553.18 mg, 10.34 mmol) and EtOH (6 mL) in H₂O (0.6 mL) was added Fe (288.77 mg, 5.17 mmol), then the mixture was stirred at 80° C. for 2 h. The reaction was filtered and concentrated under reduced pressure to give a residue. The residue was dissolved in water (10 mL) and EtOAc (3 ml), extracted with EtOAc (5 mL*3). The organic layer was dried over Na₂SO₄, concentrated to give 8-bromo-7-fluoro-4-methyl-chroman-6-amine (280 mg, crude) was obtained as a yellow solid.

### Step 7. N2-(8-bromo-7-fluoro-4-methyl-chroman-6-yl)-N4,6-dimethyl-pyrimidine-2,4-diamine

To a solution of 8-bromo-7-fluoro-4-methyl-chroman-6-amine (250 mg, 961.16 μmol) and 2-chloro-N,6-dimethyl-pyrimidin-4-amine (151.48 mg, 961.16 μmol) in i-PrOH (6 mL) was added TFA (0.1 mL), then the mixture was stirred at 140° C. for 1.5 h. The reaction was filtered to give N2-(8-bromo-7-fluoro-4-methyl-chroman-6-yl)-N4,6-dimethyl-pyrimidine-2,4-diamine (300 mg, crude) as a white solid.

### Step 7. tert-butyl 5-[7-fluoro-4-methyl-6-[[4-methyl-6-(methylamino)pyrimidin-2-yl]amino]chroman-8-yl]-2,3,4,7-tetrahydroazepine-1-carboxylate

To a solution of N2-(8-bromo-7-fluoro-4-methyl-chroman-6-yl)-N4,6-dimethyl-pyrimidine-2,4-diamine (200 mg, 524.60 μmol), tert-butyl 5-(4,4,5,5-tetramethyl-1,3,2-dioxaborolan-2-yl)-2,3,4,7-tetrahydroazepine-1-carboxylate (254.35 mg, 786.90 μmol) and Na₂CO₃ (166.81 mg, 1.57 mmol) in Tol. (2.1 mL), EtOH (0.9 mL) and H₂O (0.3 mL) was added Pd(PPh₃)₂Cl₂ (36.82 mg, 52.46 μmol), then the mixture was stirred at 100° C. for 12 h under N2. The reaction was filtered and concentrated under reduced pressure to give tert-butyl 5-[7-fluoro-4-methyl-6-[[4-methyl-6-(methylamino)pyrimidin-2-yl]amino]chroman-8-yl]-2,3,4,7-tetrahydroazepine-1-carboxylate (250 mg, crude) as a brown solid which was used for next step directly without further purification.

### Step 8. N4,6-dimethyl-N2-[rel-(4S)-7-fluoro-4-methyl-8-(2,3,4,7-tetrahydro-1H-azepin-5-yl)chroman-6-yl]pyrimidine-2,4-diamine and N4,6-dimethyl-N2-[rel-(4R)-7-fluoro-4-methyl-8-(2,3,4,7-tetrahydro-1H-azepin-5-yl)chroman-6-yl]pyrimidine-2,4-diamine

A solution of tert-butyl 5-[7-fluoro-4-methyl-6-[[4-methyl-6-(methylamino)pyrimidin-2-yl]amino]chroman-8-yl]-2,3,4,7-tetrahydroazepine-1-carboxylate (250 mg, 502.41 μmol) in HCl (4 M, 4 mL) was stirred at 25° C. for 3 h. The reaction was concentrated under reduced pressure to give the crude product. A part of it was used for next step and a part of it was purified by prep-HPLC(TFA condition:column:Phenomenex Luna 80*30 mm*3 um; mobile phase:water (TFA)-ACN; B %: 1%-30%,8 min) to give a mixture of two isomer. The mixture was separated by SFC (column: DAICEL CHIRALPAK AD(250 mm*30 mm,10 um); mobile phase: 0.1% NH₃H₂O ETOH; B %: 40%-40%;Acq Method: AD_EtOH_IPAm_40_25_35 min). N4,6-dimethyl-N2-[rel-(4S)-7-fluoro-4-methyl-8-(2,3,4,7-tetrahydro-1H-azepin-5-yl)chroman-6-yl]pyrimidine-2,4-diamine (2.9 mg, 7.30 μmol, 1.45% yield) (ee %=100%) was obtained as a pale yellow solid and N4,6-dimethyl-N2-[rel-(4R)-7-fluoro-4-methyl-8-(2,3,4,7-tetrahydro-1H-azepin-5-yl)chroman-6-yl]pyrimidine-2,4-diamine (2.2 mg, 5.53 μmol, 1.10% yield) (ee %=96.12%) was obtained as a pale yellow solid.

¹H NMR (400 MHz, METHANOL-d₄) δ: 7.96 (br s, 1H), 5.87-5.77 (m, 2H), 4.24-4.06 (m, 2H), 3.51 (br d, J=5.7 Hz, 2H), 3.19-3.11 (m, 2H), 2.96-2.91 (m, 1H), 2.90 (s, 3H), 2.57-2.48 (m, 2H), 2.16 (s, 3H), 2.10-2.04 (m, 1H), 1.90 (br s, 2H), 1.74-1.65 (m, 1H), 1.33 (d, J=7.0 Hz, 3H)

MS (ESI): m/z=398.2 [M+H]

¹H NMR (400 MHz, METHANOL-d₄) δ: 7.95 (br s, 1H), 5.88-5.73 (m, 2H), 4.24-4.06 (m, 2H), 3.50 (br d, J=5.7 Hz, 2H), 3.18-3.10 (m, 2H), 2.98-2.83 (m, 4H), 2.59-2.44 (m, 2H), 2.16 (s, 3H), 2.10-2.03 (m, 1H), 1.94-1.84 (m, 2H), 1.70 (dtd, J=3.4, 6.5, 13.5 Hz, 1H), 1.34 (d, J=7.0 Hz, 3H) MS (ESI): m/z=398.2 [M+H]

### Step 9. N4,6-dimethyl-N2-[rel-(4S)-7-fluoro-4-methyl-8-(1-methyl-2,3,4,7-tetrahydroazepin-5-yl)chroman-6-yl]pyrimidine-2,4-diamine and N4,6-dimethyl-N2-[rel-(4R)-7-fluoro-4-methyl-8-(1-methyl-2,3,4,7-tetrahydroazepin-5-yl)chroman-6-yl]pyrimidine-2,4-diamine

To a solution of N2-[7-fluoro-4-methyl-8-(2,3,4,7-tetrahydro-1H-azepin-5-yl)chroman-6-yl]-N4,6-dimethyl-pyrimidine-2,4-diamine (100 mg, 251.58 μmol) in MeOH (2 mL) was formaldehyde (15.11 mg, 503.16 μmol, 13.95 μL), then the mixture was added sodium;cyanoboranuide (79.05 mg, 1.26 mmol) and stirred at 25° C. for 12 h. The reaction was concentrated under reduced pressure to give the residue. The crude product was purified by prep-HPLC(TFA condition:column:Phenomenex Luna 80*30 mm*3 um; mobile phase:water (TFA)-ACN; B %: 5%-35%,8 min) to give the residue which was separated by Chiral SFC (column: DAICEL CHIRALCEL OX (250 mm*30 mm,10 um); mobile phase: 0.1% NH₃H₂O IPA; B %: 60%-60%; Acq Method: IE_H_I_ACN_IPAm_20_13_30). N4,6-dimethyl-N2-[rel-(4S)-7-fluoro-4-methyl-8-(1-methyl-2,3,4,7-tetrahydroazepin-5-yl)chroman-6-yl]pyrimidine-2,4-diamine (24.7 mg, 60.02 μmol, 23.86% yield) (ee %=99.16%; Rt=2.230) was obtained as a pale yellow solid N4,6-dimethyl-N2-[rel-(4R)-7-fluoro-4-methyl-8-(1-methyl-2,3,4,7-tetrahydroazepin-5-yl)chroman-6-yl]pyrimidine-2,4-diamine (24.7 mg, 60.02 μmol, 23.86% yield) (ee %=97.58%; Rt=2.582) was obtained as a pale yellow solid.

¹H NMR (400 MHz, METHANOL-d₄) δ: 7.71 (br d, J=8.6 Hz, 1H), 5.91 (s, 1H), 5.79 (t, J=6.7 Hz, 1H), 4.26-4.15 (m, 2H), 3.99 (d, J=6.7 Hz, 2H), 3.58-3.51 (m, 2H), 3.00-2.93 (m, 1H), 2.90 (d, J=6.0 Hz, 6H), 2.69-2.61 (m, 2H), 2.23 (s, 3H), 2.15-2.04 (m, 3H), 1.74-1.73 (m, 1H), 1.34 (d, J=7.0 Hz, 3H)

MS (ESI): m/z=412.2 [M+H]

¹H NMR (400 MHz, METHANOL-d₄) δ: 8.14-7.79 (m, 1H), 5.83-5.78 (m, 1H), 5.76-5.70 (m, 1H), 4.23-4.08 (m, 2H), 3.46-3.40 (m, 2H), 3.07-3.01 (m, 2H), 2.96-2.85 (m, 4H), 2.51 (br d, J=5.3 Hz, 5H), 2.21-2.13 (m, 3H), 2.06 (br d, J=3.5 Hz, 1H), 1.94-1.89 (m, 2H), 1.75-1.63 (m, 1H), 1.35-1.30 (m, 3H)

MS (ESI): m/z=412.2 [M+H]

### EXAMPLE 86. Synthesis of Compound 434 and Compound 428

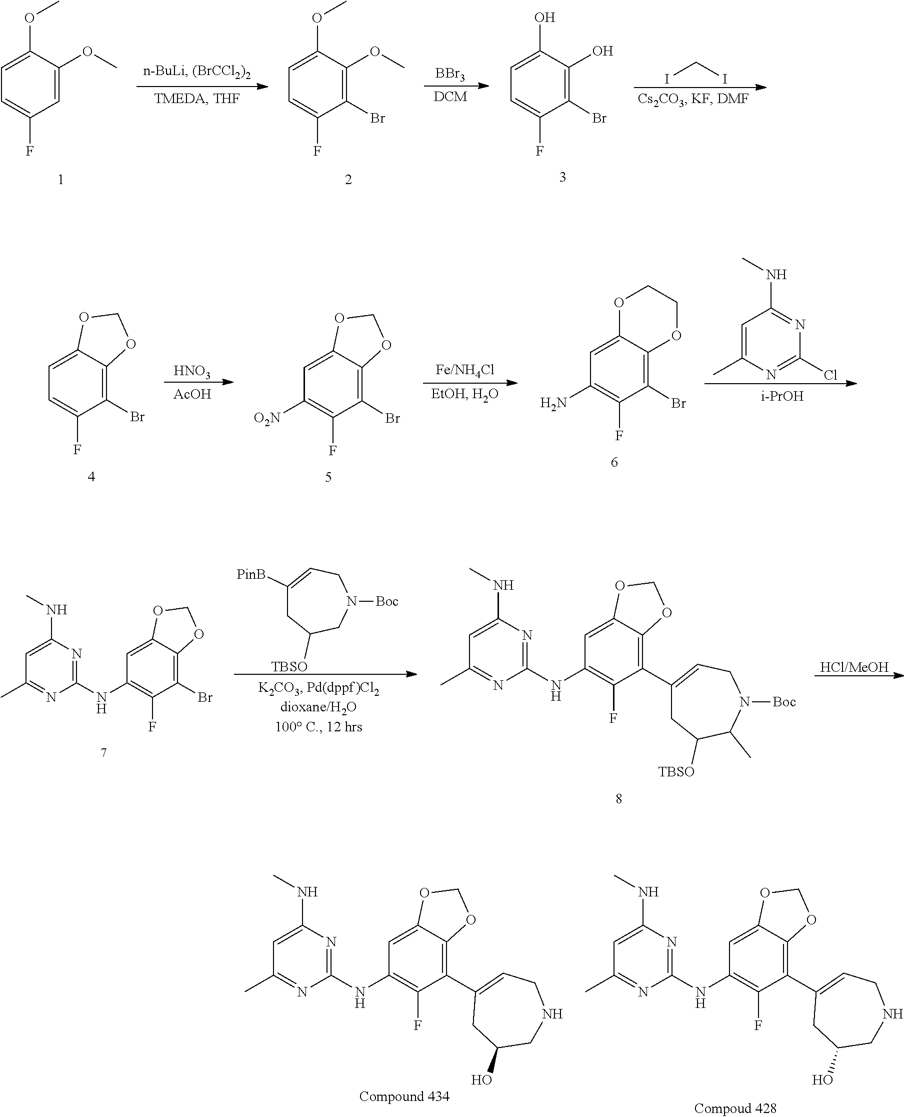

### Step 1. 2-bromo-1-fluoro-3,4-dimethoxy-benzene

n-butyllithium (2.5 M, 38.42 mL) was added dropwise at −78° C. to a solution of compound 4-fluoro-1,2-dimethoxy-benzene (10 g, 64.04 mmol, 8.54 mL) and N,N,N′,N′-tetramethylethane-1,2-diamine (7.44 g, 64.04 mmol, 9.60 mL) in THF (128 mL) under nitrogen atmosphere and stirred at −78° C. for 1.5 h. Then 1,2-dibromo-1,1,2,2-tetrachloro-ethane (27.32 g, 83.89 mmol, 10.07 mL) in THF (32 mL) was added, after stirring for a further 10 min, the cooling bath was removed and the reaction vessel allowed to warm to room temperature. The reaction mixture was quenched by sat. NH₄Cl (100 mL), extracted with EtOAc (300 mL*3). The organic layer was dried over Na₂SO₄, concentrated to give the crude product. The crude product was purified by column chromatography on silica gel to give 2-bromo-1-fluoro-3,4-dimethoxy-benzene (15 g, crude) as a yellow gum.

### Step 2. 3-bromo-4-fluoro-benzene-1,2-diol

To a mixture of 2-bromo-1-fluoro-3,4-dimethoxy-benzene (14.5 g, 61.69 mmol) in DCM (160 mL) was added BBr₃ (77.27 g, 308.45 mmol) in DCM (160 mL) at 0° C., then the mixture was stirred at 25° C. for 3 hrs. The reaction mixture was quenched by MeOH (150 mL) at 0° C. The reaction mixture was filtered and concentrated to give 3-bromo-4-fluoro-benzene-1,2-diol (10 g, crude) as a brown gum.

### Step 3. 4-bromo-5-fluoro-1,3-benzodioxole

To a mixture of 3-bromo-4-fluoro-benzene-1,2-diol (4.5 g, 21.74 mmol) in DMF (60 mL) was added C_{S2}CO₃ (21.25 g, 65.22 mmol), KF (1.26 g, 21.74 mmol), diiodomethane (11.65 g, 43.48 mmol, 3.50 mL) at 25° C., then the mixture was stirred at 100° C. for 12 hrs. The reaction mixture was added to water (150 mL), extracted with EtOAc (500 mL*3). The combined organic layers were washed with 500 mL of brine, dried over Na₂SO₄, filtered and concentrated under reduced pressure to give a residue. The crude product was purified by column chromatography on silica gel to give 4-bromo-5-fluoro-1,3-benzodioxole (3.3 g, crude) as a white solid.

### Step 4. 4-bromo-5-fluoro-6-nitro-1,3-benzodioxole

To a mixture of HNO₃ (2.09 g, 22.60 mmol, 68% purity) in AcOH (33 mL) was added 4-bromo-5-fluoro-1,3-benzodioxole (3.3 g, 15.07 mmol) at 0° C., then the mixture was stirred at 25° C. for 12 hrs. This reaction was set four batch in parallel. The reaction mixture was added to ice water (100 mL), the mixture was filtered and the filter cake was dried in vacuum to give 4-bromo-5-fluoro-6-nitro-1,3-benzodioxole (2.34 g, crude) as a yellow solid.

### Step 5. 7-bromo-6-fluoro-1,3-benzodioxol-5-amine

To a mixture of 7-bromo-6-methyl-5-nitro-2,3-dihydrobenzofuran (1 g, 3.87 mmol) in EtOH (20 mL), H₂O (2 mL) was added Fe (1.08 g, 19.37 mmol), NH₄Cl (2.07 g, 38.75 mmol) at 25° C., then the mixture was stirred at 80° C. for 12 hrs. The reaction mixture was filtered and concentrated to give the crude product. The crude product was added to water (50 mL), extracted with EtOAc (50 mL*3). The combined organic layers were washed with 10 mL of brine, dried over anhydrous Na₂SO₄, filtered and concentrated under reduced pressure to give the crude product. The crude product was purified by prep-TLC to give 7-bromo-6-fluoro-1,3-benzodioxol-5-amine (0.9 g, crude) as a yellow solid.

### Step 6. N2-(7-bromo-6-fluoro-1,3-benzodioxol-5-yl)-N4,6-dimethyl-pyrimidine-2,4-diamine

To a solution of 7-bromo-6-fluoro-1,3-benzodioxol-5-amine (310 mg, 1.32 mmol) and 2-chloro-N,6-dimethyl-pyrimidin-4-amine (208.77 mg, 1.32 mmol) in i-PrOH (6 mL) was added TFA (10 mg, 87.70 mol, 6.76 L), then the mixture was stirred at 140° C. for 2 hrs. The reaction was concentrated under reduced pressure to give N2-(7-bromo-6-fluoro-1,3-benzodioxol-5-yl)-N4,6-dimethyl-pyrimidine-2,4-diamine (240 mg, crude) as brown solid.

### Step 7. tert-butyl 3-[tert-butyl(dimethyl)silyl]oxy-5-[5-fluoro-6-[[4-methyl-6-(methylamino)pyrimidin-2-yl]amino]-1,3-benzodioxol-4-yl]-2,3,4,7-tetrahydroazepine-1-carboxylate

To a mixture of N2-(7-bromo-6-fluoro-1,3-benzodioxol-5-yl)-N4,6-dimethyl-pyrimidine-2,4-diamine (80 mg, 225.25 mol) and tert-butyl 3-[tert-butyl (dimethyl) silyl]oxy-5-(4,4,5,5-tetramethyl-1,3,2-dioxaborolan-2-yl)-2, 3, 4, 7-tetrahydroazepine-1-carboxylate (102.15 mg, 225.25 μmol) indioxane (2 mL) and H₂O (0.2 mL) was added K₂CO₃ (93.39 mg, 675.75 μmol) and cyclopentyl (diphenyl) phosphane; dichloropalladium;iron (16.48 mg, 22.52 μmol) under N₂ atmosphere. It was stirred at 100° C. for 12 hrs under N₂ atmosphere. The reaction mixture was concentrated to give Tert-butyl 3-[tert-butyl(dimethyl) silyl]oxy-5-[5-fluoro-6-[[4-methyl-6-(methylamino) pyrimidin-2-yl]amino]-1,3-benzodioxol-4-yl]-2,3,4,7-tetrahydroazepine-1-carboxylate (130 mg, crude) as a white solid.

### Step 8. rel-(3S)-5-[5-fluoro-6-[[4-methyl-6-(methylamino)pyrimidin-2-yl]amino]-1,3-benzodioxol-4-yl]-2,3,4,7-tetrahydro-1H-azepin-3-ol and rel-(3R)-5-[5-fluoro-6-[[4-methyl-6-(methylamino)pyrimidin-2-yl]amino]-1,3-benzodioxol-4-yl]-2,3,4,7-tetrahydro-1H-azepin-3-ol

A solution of tert-butyl 3-[tert-butyl(dimethyl)silyl]oxy-5-[5-fluoro-6-[[4-methyl-6-(methylamino)pyrimidin-2-yl]amino]-1,3-benzodioxol-4-yl]-2,3,4,7-tetrahydroazepine-1-carboxylate (130 mg, 216.02 μmol) in HCl (4.0 M in Methanol) was stirred at 25° C. for 1 h. The reaction was concentrated under reduced pressure to give a residue. The residue was purified by prep-HPLC (TFA condition, column: Phenomenex Luna 80*30 mm*3 um; mobile phase: [water (TFA)-ACN; B %: 1%-25%, 8 min) to give crude product. The crude product was purified by Chiral SFC (Column: DAICEL CHIRALPAK IG(250 mm*30 mm,10 um); Mobile phase: A for Heptane and B for MEOH(0.1% NH₃H₂O); Gradient: B %=45-45%; Flow rate:70 ml/min, to give rel-(3S)-5-[5-fluoro-6-[[4-methyl-6-(methylamino)pyrimidin-2-yl]amino]-1,3-benzodioxol-4-yl]-2,3,4,7-tetrahydro-1H-azepin-3-ol (4.8 mg, 12.39 μmol, 5.74% yield) as a light-yellow solid and rel-(3R)-5-[5-fluoro-6-[[4-methyl-6-(methylamino)pyrimidin-2-yl]amino]-1,3-benzodioxol-4-yl]-2,3,4,7-tetrahydro-1H-azepin-3-ol (8.1 mg, 20.91 μmol, 9.68% yield) as a yellow solid.

¹H NMR (400 MHz, METHANOL-d₄) δ: 7.81 (d, J=6.6 Hz, 1H), 6.19 (t, J=6.1 Hz, 1H), 5.97-5.90 (m, 2H), 5.83 (s, 1H), 3.96 (br s, 1H), 3.58 (t, J=6.4 Hz, 2H), 3.40 (dd, J=2.6, 13.2 Hz, 1H), 3.03-2.93 (m, 2H), 2.88 (s, 4H), 2.82-2.72 (m, 1H), 2.17 (s, 3H)

¹H NMR (400 MHz, METHANOL-d₄) δ: 7.94-7.65 (m, 1H), 6.19 (t, J=6.0 Hz, 1H), 5.94 (d, J=3.5 Hz, 2H), 5.83 (s, 1H), 4.02-3.89 (m, 1H), 3.67-3.50 (m, 2H), 3.39 (dd, J=2.8, 13.0 Hz, 1H), 3.03-2.90 (m, 2H), 2.88 (s, 3H), 2.77 (br d, J=14.9 Hz, 1H), 2.17 (s, 3H)

### EXAMPLE 87. Synthesis of Compound 435 and Compound 436

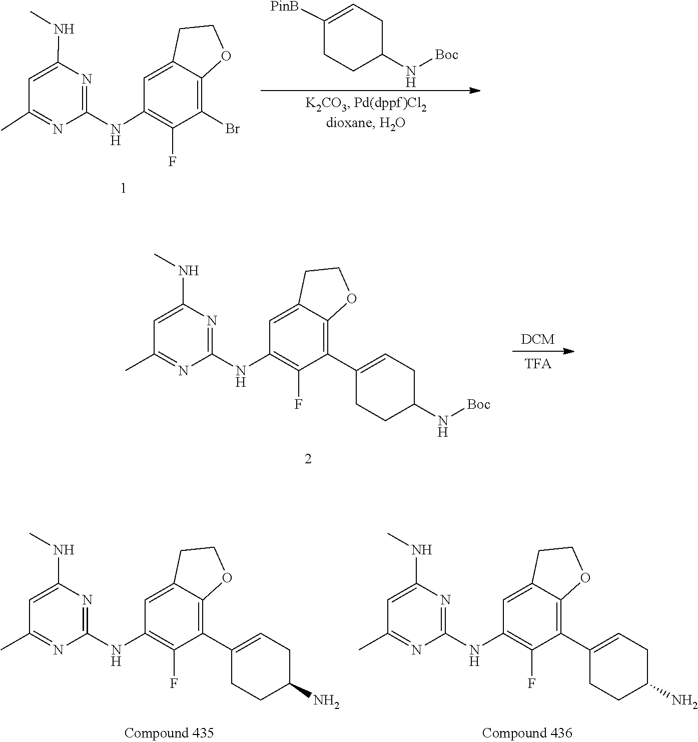

### Step 1. tert-butyl N-[4-[6-fluoro-5-[[4-methyl-6-(methylamino) pyrimidin-2-yl]amino]-2,3-dihydrobenzofuran-7-yl]cyclohex-3-en-1-yl]carbamate

To a solution of N2-(7-bromo-6-fluoro-2,3-dihydrobenzofuran-5-yl)-N4,6-dimethyl-pyrimidine-2,4-diamine (80 mg, 226.51 μmol), tert-butyl N-[4-(4,4,5,5-tetramethyl-1,3,2-dioxaborolan-2-yl)cyclohex-3-en-1-yl]carbamate (80.54 mg, 249.16 μmol), K₂CO₃ (156.52 mg, 1.13 mmol) and H₂O (0.3 mL) in dioxane (3 mL) was added cyclopentyl (diphenyl) phosphane;dichloropalladium;iron (16.57 mg, 22.65 μmol), then the mixture was stirred at 100° C. for 12 h under N2. The reaction was filtered and concentrated under reduced pressure to give tert-butyl N-[4-[6-fluoro-5-[[4-methyl-6-(methylamino) pyrimidin-2-yl]amino]-2,3-dihydrobenzofuran-7-yl]cyclohex-3-en-1-yl]carbamate (106 mg, crude) as a black solid.

### Step 2. N2-[6-fluoro-7-[rac-(4S)-4-aminocyclohexen-1-yl]-2,3-dihydrobenzofuran-5-yl]-N4,6-dimethyl-pyrimidine-2,4-diamine and N2-[6-fluoro-7-[rac-(4R)-4-aminocyclohexen-1-yl]-2,3-dihydrobenzofuran-5-yl]-N4,6-dimethyl-pyrimidine-2,4-diamine

To a solution of tert-butyl N-[4-[6-fluoro-5-[[4-methyl-6-(methylamino) pyrimidin-2-yl]amino]-2,3-dihydrobenzofuran-7-yl]cyclohex-3-en-1-yl]carbamate (106 mg, 225.75 μmol) in DCM (2 mL) was added TFA (1 mL), then the mixture was stirred at 25° C. for 1 h.

The reaction mixture was filtered and purified by prep-HPLC(TFA condition:column: Phenomenex Luna 80*30 mm*3 um; mobile phase:water(TFA)-ACN; B %: 1%-30%,8 min) to give the mixture of these two single isomer. The mixture was separated by SFC (column: DAICEL CHIRALPAKIC (250 mm*30 mm,10 um); mobile phase:[0.1% NH₃H₂O MEOH]; B %: 50%-55%; SFC), Acq Method: IC_MeOH_IPAm_5_50_3_35_3 min) to give N2-[6-fluoro-7-[rac-(4S)-4-aminocyclohexen-1-yl]-2,3-dihydrobenzofuran-5-yl]-N4,6-dimethyl-pyrimidine-2,4-diamine (7.7 mg, 20.84 μmol, 9.23% yield) (13.6 mg, 35.28 μmol, 15.86% yield, Rt=3.020 min) as a pale yellow solid and N2-[6-fluoro-7-[rac-(4R)-4-aminocyclohexen-1-yl]-2,3-dihydrobenzofuran-5-yl]-N4,6-dimethyl-pyrimidine-2,4-diamine (6.2 mg, 16.78 μmol, 7.43% yield, Rt=2.692 m) as a pale yellow solid.

¹HNMR (400 MHz, METHANOL-d₄) δ:1.58-1.67 (m, 1H) 1.90-1.99 (m, 1H) 2.06 (s, 4H)2.30-2.51 (m, 3H) 2.76 (s, 3H) 3.08 (br t, J=8.58 Hz, 2 H) 3.19 (br s, 1H)4.44 (J=8.76 Hz, 2H) 4.76 (s, 16H) 5.62-5.76 (m, 2H) 7.76 (br d, J=7.39 Hz, 1H)

¹HNMR (400 MHz, METHANOL-d₄) δ:1.46-1.62 (m, 1H) 1.85-2.02 (m, 2H) 2.06 (s, 3H)2.22-2.45 (i, 3H) 2.76 (s, 3H) 2.99-3.14(m, 3H) 3.21 (d J=3.28, 1.58 Hz, 6H) 4.44 (t, J=8.76 Hz, 2H) 4.76 (s, 12H) 5.63-5.70 (m, 2H) 7.74 (br d, J=7.75 Hz, 1H)

### EXAMPLE 88. Additional Exemplary Compounds

The compounds in Table 1A below were synthesized and characterized using methods similar to those described herein.

TABLE 1A
Analytical data for additional examplary compounds

| Compound | | |
|---|---|---|
| No. | HNMR data | LCMS data |
| 169 | 1H NMR (400 MHz, METHANOL-d4) δ: 7.45 (br d, J = 7.39 Hz, 1 H) 5.93 (t, | MS (ESI): |
| | J = 6.26 Hz, 1 H) 5.82-5.89 (m, 1 H) 4.58-4.73 (m, 2 H) 3.98-4.10 (m, 1 | m/z = 384.2[M + H] |
| | H) 3.81 (d, J = 6.44 Hz, 2 H) 3.27-3.51 (m, 4 H) 2.79 (s, 3 H) 2.61-2.67 (m, | |
| | 2 H) 2.16 (s, 3 H) 1.91-2.03 (m, 2 H) 1.22 (d, J = 6.79 Hz, 3 H) | |
| 171 | 1H NMR (400 MHz, METHANOL-d4) δ: 7.00 (br d, J = 7.21 Hz, 1 H) 5.95- | MS (ESI): |
| | 6.05 (m, 2 H) 4.58 (s, 2 H) 4.10 (br d, J = 4.65 Hz, 1 H) 4.02 (br d, J = 6.97 | m/z = 410.2[M + H] |
| | Hz, 1 H) 3.69 (br s, 1 H) 3.47 (br s, 1 H) 2.94 (s, 3 H) 2.88 (s, 3 H) 2.77 (br d, | |
| | J = 6.48 Hz, 2 H) 2.28 (s, 3 H) 2.12 (br s, 2 H) 1.08 (br d, J = 10.02 Hz, 4 H) | |
| 172 | 1H NMR (400 MHz, METHANOL-d4) δ: 6.99 (br d, J = 7.21 Hz, 1 H) 6.07 | MS (ESI): |
| | (t, J = 6.36 Hz, 1 H) 5.97 (d, J = 0.61 Hz, 1 H) 4.57 (s, 2 H) 3.93 (d, J = 6.36 | m/z = 396.2[M + H] |
| | Hz, 2 H) 3.46-3.52 (m, 2 H) 2.88 (s, 3 H) 2.72-2.82 (m, 2 H) 2.28 (s, 3 H) | |
| | 2.05-2.14 (m, 2 H) 1.08 (br d, J = 10.39 Hz, 4 H) | |
| 173 | 1H NMR (400 MHz, METHANOL-d4) δ: 8.12 (d, J = 8.33 Hz, 1 H) 6.03 (d, | MS (ESI): m/z = |
| | J = 0.88 Hz, 1 H) 5.87 (t, J = 6.58 Hz, 1 H) 4.39-4.49 (m, 2 H) 3.97-4.14 | 434.2[M + H] |
| | (m, 2 H) 3.63-3.78 (m, 1 H) 3.42-3.55 (m, 1 H) 2.94 (d, J = 10.96 Hz, 6 H) | |
| | 2.61-2.82 (m, 2 H) 2.48-2.58 (m, 2 H) 2.32 (s, 3 H) 2.12 (br d, J = 5.26 Hz, | |
| | 2 H) | |
| 174 | 1H NMR (400 MHz, METHANOL-d4) δ: 8.11 (d, J = 8.33 Hz, 1 H) 6.03 (d, | MS (ESI): m/z = |
| | J = 0.66 Hz, 1 H) 5.93 (t, J = 6.25 Hz, 1 H) 4.40-4.46 (m, 2 H) 3.92 (d, J = | 420.1[M + H] |
| | 6.36 Hz, 1 H) 3.45-3.54 (m, 2 H) 2.92 (s, 3 H) 2.64-2.72 (m, 2 H) 2.46- | |
| | 2.60 (m, 2 H) 2.31 (s, 3 H) 2.04-2.14 (m, 2 H) | |
| 175 | 1H NMR (400 MHz, METHANOL-d4) δ: 7.42-7.34 (m, 1H) 6.02-5.92 (m, | MS (ESI): m/z = |
| | 1H) 5.79-5.69 (m, 1H) 4.26-4.17 (m, 2H) 3.97-3.87 (m, 2H) 3.57-3.38 | 400.1 [M + H] |
| | (m, 2H) 2.91 (s, 3H) 2.86-2.80 (m, 2H) 2.71-2.53 (m, 2H) 2.34-2.21 (m, | |
| | 3H) 2.19-2.07 (m, 2H) 2.05-1.96 (m, 2H) | |
| 176 | 1H NMR (400 MHz, METHANOL-d4) δ: 7.41-7.33 (m, 1H) 6.02-5.92 (m, | MS (ESI): m/z = |
| | 1H) 5.74-5.58 (m, 1H) 4.28-4.20 (m, 2H) 4.15-3.93 (m, 2H) 3.76-3.42 | 414.1 [M + H] |
| | (m, 1H) 2.97 (s, 3H) 2.93-2.89 (m, 3H) 2.86-2.80 (m, 2H) 2.74-2.64 (m, | |
| | 1H) 2.63-2.49 (m, 1H) 2.27 (s, 3H) 2.24-2.07 (m, 2H) 2.05-1.98 (m, 2H) | |
| 178 | 1H NMR (400 MHz, METHANOL-d4) δ: 8.65-8.39 (m, 2H) 7.63 (br d, J = | MS (ESI): m/z = |
| | 6.8 Hz, 1H) 6.08-6.01 (m, 1H) 5.94-5.88 (m, 1H) 4.32-4.27 (m, 2H) 3.94- | 398.1 [M + H] |
| | 3.89 (m, 2H) 3.51-3.44 (m, 2H) 2.91-2.87 (m, 3H) 2.79-2.72 (m, 2H) | |
| | 2.26-2.23 (m, 3H) 2.12-2.04 (m, 2H) 1.37-1.33 (m, 6H) | |
| 180 | 1H NMR (400 MHz, METHANOL-d4) δ: 7.47-7.32 (m, 1H) 6.22-6.05 (m, | MS (ESI): m/z = |
| | 1H) 6.04-5.94 (m, 1H) 4.74-4.62 (m, 2H) 4.33-4.11 (m, 1H) 4.11-3.86 | 400.1 [M + H] |
| | (m, 2H) 3.77-3.33 (m, 2H) 3.29-3.23 (m, 2H) 2.98 (br s, 3H) 2.93 (s, 2H) | |
| | 2.90 (s, 3H) 2.44-2.18 (m, 3H) | |
| 181 | 1H NMR (400 MHz, METHANOL-d4) δ: 7.48-7.41 (m, 1H) 6.02-5.97 (m, | MS (ESI): m/z = |
| | 2H) 4.81-4.79 (m, 1H) 4.21-4.16 (m, 1H) 4.12-4.07 (m, 1H) 4.01 (br d, J = | 398.1 [M + H] |
| | 6.9 Hz, 1H) 3.72-3.66 (m, 1H) 3.62-3.56 (m, 1H) 3.50-3.47 (m, 1H) 2.94 | |
| | (s, 3H) 2.91 (s, 3H) 2.81-2.71 (m, 2H) 2.29 (s, 3H) 2.18-2.06 (m, 2H) 1.33 | |
| | (br d, J = 6.5 Hz, 3H) | |
| 182 | 1H NMR (400 MHz, METHANOL-d4) δ: 7.89-7.79 (m, 1H) 5.81-5.78 (m, | MS (ESI): m/z = |
| | 1H) 5.76-5.72 (m, 1H) 4.59-4.52 (m, 2H) 3.82-3.71 (m, 1H) 3.36-3.34 | 384.1 [M + H] |
| | (m, 1H) 3.22-3.15 (m, 2H) 3.11-3.01 (m, 1H) 2.87-2.85 (m, 3H) 2.74- | |
| | 2.64 (m, 1H) 2.57-2.47 (m, 1H) 2.18-2.14 (m, 3H) 1.91-1.83 (m, 2H) 1.31- | |
| | 1.29 (m, 3H) | |
| 185 | 1H NMR (400 MHz, METHANOL-d4) δ: 7.83 (br d, J = 7.2 Hz, 1H) 5.81- | MS (ESI): m/z = |
| | 5.77 (m, 1H) 5.76-5.71 (m, 1H) 4.55 (dt, J = 3.7, 8.7 Hz, 2H) 3.73-3.64 (m, | 384.1 [M + H] |
| | 1H) 3.30-3.25 (m, 1H) 3.22-3.14 (m, 2H) 3.07-2.97 (m, 1H) 2.87-2.83 | |
| | (m, 3H) 2.72-2.62 (m, 1H) 2.55-2.45 (m, 1H) 2.19-2.13 (m, 2H) 1.90- | |
| | 1.78 (m, 2H) 1.30-1.25 (m, 3H) | |
| 186 | 1H NMR (400 MHz, METHANOL-d4) δ: 6.21-5.99 (m, 1H) 5.99-5.92 (m, | MS (ESI): m/z = |
| | 1H) 4.78-4.69 (m, 2H) 4.16-3.93 (m, 2H) 3.79-3.58 (m, 1H) 3.34 (br s, | 402.1 [M + H] |
| | 2H) 3.30-3.24 (m, 1H) 2.97-2.91 (m, 3H) 2.91-2.81 (m, 3H) 2.78-2.64 | |
| | (m, 2H) 2.42-2.27 (m, 3H) 2.15-2.03 (m, 2H) | |
| 187 | 1H NMR (400 MHz, METHANOL-d4) δ: 6.20-6.01 (m, 1H) 6.01-5.98 (m, | MS (ESI): m/z = |
| | 1H) 4.77-4.66 (m, 2H) 3.95-3.84 (m, 2H) 3.51-3.42 (m, 2H) 3.32 (br s, | 388.1 [M + H] |
| | 1H) 3.30-3.24 (m, 1H) 3.06-2.80 (m, 3H) 2.77-2.68 (m, 2H) 2.40-2.27 | |
| | (m, 3H) 2.13-1.98 (m, 2H) | |
| 190 | 1H NMR (400 MHz, METHANOL-d4) δ: 7.31 (br d, J = 8.38 Hz, 1 H) 5.98 | MS (ESI): m/z = |
| | (s, 1 H) 5.80 (br t, J = 6.57 Hz, 1 H) 4.17-4.27 (m, 2 H) 4.03 (br s, 2 H) 3.41- | 398.1 [M + H] |
| | 3.70 (m, 2 H) 2.91 (d, J = 15.13 Hz, 6 H) 2.80 (br t, J = 6.19 Hz, 2 H) 2.66 (br s, | |
| | 2 H) 2.28 (s, 3 H) 2.12 (br d, J = 4.88 Hz, 2 H) 1.96-2.03 (m, 2 H) | |
| 191 | 1H NMR (400 MHz, METHANOL-d4) δ: 7.22-7.28 (m, 1 H) 6.00 (s, 1 H) | MS (ESI): m/z = |
| | 5.92 (t, J = 6.25 Hz, 1 H) 4.30 (br d, J = 5.00 Hz, 2 H) 4.26 (br d, J = 5.13 Hz, 2 | 386.1 [M + H] |
| | H) 3.92 (d, J = 6.25 Hz, 2 H) 3.47-3.51 (m, 2 H) 2.93 (s, 3 H) 2.67-2.72 (m, | |
| | 2 H) 2.29 (s, 3 H) 2.05-2.13 (m, 2 H) | |
| 192 | 1H NMR (400 MHz, METHANOL-d4) δ: 7.75-7.58 (m, 1H) 6.07-5.90 (m, | MS (ESI): m/z = |
| | 2H) 4.63-4.56 (m, 2H) 3.92 (d, J = 6.4 Hz, 2H) 3.69 (s, 3H) 3.51-3.46 (m, | 381.2 [M + H] |
| | 2H) 3.23 (br t, J = 8.6 Hz, 2H) 2.97 (s, 3H) 2.79-2.71 (m, 2H) 2.27 (s, 3H) | |
| | 2.15-2.05 (m, 2H) | |
| 194 | 1H NMR (400 MHz, METHANOL-d4) δ: 7.38 (d, J = 7.5 Hz, 1H) 6.05 | MS (ESI): m/z = |
| | (t, J = 6.4 Hz, 1H) 4.67 (t, J = 8.8 Hz, 2H) 3.94 (d, J = 6.4 Hz, 2H) 3.74 (s, 3H) | 400.1 [M + H] |
| | 3.53-3.46 (m, 2H) 3.26 (t, J = 8.7 Hz, 2H) 2.96 (s, 3H) 2.80-2.71 (m, 2H) | |
| | 2.34-2.27 (m, 3H) 2.14-2.05 (m, 2H) | |
| 195 | 1H NMR (400 MHz, METHANOL-d4) δ: 9.91 (br s, 1H) 8.51 (br s, 1H) 7.42 | MS (ESI): m/z = |
| | (d, J = 7.5 Hz, 1H) 6.05 (s, 1H) 5.91 (t, J = 6.6 Hz, 1H) 4.63 (t, J = 8.7 Hz, | 412.2 [M + H] |
| | 2H) 3.98 (br d, J = 6.4 Hz, 2H) 3.62-3.54 (m, 1H) 3.48 (br s, 2H) 3.22 (br t, | |
| | J = 8.7 Hz, 2H) 2.81 (s, 3H) 2.71-2.65 (m, 2H) 2.27 (s, 3H) 2.07-1.99 (m, | |
| | 2H) 1.31 (d, J = 6.5 Hz, 6H) | |
| 196 | 1H NMR (400 MHz, METHANOL-d4) δ: 7.27 (d, J = 7.5 Hz, 1H) 5.88 | MS (ESI): m/z = |
| | (t, J = 6.7 Hz, 1H) 4.56 (dt, J = 2.1, 8.7 Hz, 2H) 4.04-3.85 (m, 2H) 3.62 (s, 3H) | 414.1 [M + H] |
| | 3.60-3.54 (m, 1H) 3.40-3.31 (m, 1H) 3.15 (t, J = 8.8 Hz, 2H) 2.85-2.83 | |
| | (m, 6H) 2.72-2.58 (m, 2H) 2.21 (s, 3H) 2.06-1.94 (m, 2H) | |
| 197 | 1H NMR (400 MHz, METHANOL-d4) δ: 7.24 (br d, J = 7.50 Hz, 1 H) 5.92 | MS (ESI): m/z = |
| | (t, J = 6.32 Hz, 1 H) 4.28-4.31 (m, 2 H) 4.24-4.27 (m, 2 H) 3.92 (d, J = | 404.1 [M + H] |
| | 6.25 Hz, 2 H) 3.46-3.51 (m, 2 H) 2.99 (s, 3 H) 2.67-2.72 (m, 2 H) 2.32 (d, | |
| | J = 2.88 Hz, 3 H) 2.05-2.13 (m, 2 H) | |
| 198 | 1H NMR (400 MHz, METHANOL-d4) δ: 7.33-7.21 (m, 1H) 5.97-5.90 (m, | MS (ESI): m/z = |
| | 2H) 4.61-4.52 (m, 2H) 3.88 (d, J = 6.4 Hz, 2H) 3.50-3.44 (m, 2H) 3.21- | 368.1 [M + H] |
| | 3.12 (m, 2H) 2.93 (s, 3H) 2.73-2.67 (m, 2H) 2.25 (s, 3H) 2.13-2.05 (m, | |
| | 2H) | |
| 199 | 1H NMR (400 MHz, METHANOL-d4) δ: 7.32-7.46 (m, 1 H) 6.14-6.58 | MS (ESI): m/z = |
| | (m, 1 H) 5.88-6.06 (m, 2 H) 4.63-4.71 (m, 2 H) 4.16 (brd, J = 6.63 Hz, 2 H) | 434.1 [M + H] |
| | 3.58-3.83 (m, 4 H) 3.26 (br t, J = 8.63 Hz, 2 H) 2.85-2.96 (m, 3 H) 2.79 (br | |
| | d, J = 4.75 Hz, 2 H) 2.28 (s, 3 H) 2.10 (br s, 2 H) | |
| 200 | 1H NMR (400 MHz, METHANOL-d4) δ: 7.42-7.36 (m, 1H) 5.99-5.96 (m, | MS (ESI): m/z = |
| | 1H) 5.61-5.56 (m, 1H) 4.73-4.64 (m, 3H) 3.76-3.67 (m, 1H) 3.59-3.51 | 398.1 [M + H] |
| | (m, 1H) 3.29-3.23 (m, 2H) 3.01-2.94 (m, 1H) 2.93 (s, 3H) 2.87-2.81 (m, | |
| | 3H) 2.62-2.54 (m, 1H) 2.33 (br s, 3H) 2.24-2.11 (m, 1H) 1.94-1.84 (m, | |
| | 1H) 1.58-1.51 (m, 3H) | |
| 201 | 1H NMR (400 MHz, METHANOL-d4) δ: 7.39 (br d, J = 7.38 Hz, 1 H) 5.96- | MS (ESI): m/z = |
| | 6.01 (m, 2 H) 4.66 (td, J = 8.66, 2.19 Hz, 2 H) 4.06 (d, J = 6.75 Hz, 2 H) 3.64- | 398.1 [M + H] |
| | 3.72 (m, 1 H) 3.44-3.53 (m, 1 H) 3.20-3.30 (m, 4 H)2.89 (s, 3 H) 2.77 | |
| | (br. s, 2 H) 2.28 (s, 3 H) 2.04-2.16 (m, 2 H) 1.36 (t, J = 7.25 Hz, 3 H) | |
| 203 | 1H NMR (400 MHz, METHANOL-d4) δ: 7.18 (br. d, J = 6.25 Hz, 1 H) 6.07- | MS (ESI): m/z = |
| | 6.11 (m, 1 H) 6.05 (s, 2 H) 6.00 (s, 1 H) 4.01-4.13 (m, 2 H) 3.59-3.69 (m, | 386.2 [M + H] |
| | 1 H) 3.45-3.56 (m, 1 H) 2.94 (s, 3 H) 2.92 (s, 3 H) 2.80 (br. s, 2 H) 2.29 (s, | |
| | 3 H) 2.11 (br. s, 2 H) | |
| 204 | 1H NMR (400 MHz, METHANOL-d4) δ: 7.38 (d, J = 7.0 Hz, 1H) 5.99- | MS (ESI): m/z = |
| | 5.96 (m, 1H) 5.95-5.89 (m, 1H) 4.73-4.58 (m, 2H) 4.18-3.95 (m, 2H) 3.92- | 398.1 [M + H] |
| | 3.78 (m, 1H) 3.70-3.57 (m, 1H) 3.30-3.21 (m, 2H) 3.00-2.86 (m, 4H) | |
| | 2.69-2.55 (m, 1H) 2.39-2.27 (m, 3H) 2.16-1.76 (m, 2H) 1.57-1.47 (m, | |
| | 1H) 1.38 (d, J = 6.1 Hz, 2H) | |
| 205 | 1H NMR (400 MHz, METHANOL-d4) δ: 7.18 (d, J = 6.4 Hz, 1H) 6.11- | MS (ESI): m/z = |
| | 6.07 (m, 1H) 6.05 (s, 2H) 4.18-4.09 (m, 1H) 4.06-3.98 (m, 1H) 3.73-3.66 | 404.1 [M + H] |
| | (m, 1H) 3.48 (dt, J = 1.9, 4.5 Hz, 1H) 2.98 (s, 3H) 2.94 (s, 3H) 2.80 (br. s, | |
| | 2H) 2.32 (d, J = 3.0 Hz, 3H) 2.17-2.05 (m, 2H) | |
| 206 | 1H NMR (400 MHz, METHANOL-d4) δ: 7.42-7.34 (m, 1H) 6.00-5.95 (m, | MS (ESI): m/z = |
| | 1H) 5.77-5.72 (m, 1H) 4.70-4.61 (m, 2H) 4.36-4.28 (m, 1H) 3.63-3.53 | 384.1 [M + H] |
| | (m, 1H) 3.43-3.35 (m, 1H) 3.28-3.20 (m, 2H) 2.93 (br. s, 1H) 2.87-2.78 | |
| | (m, 1H) 2.67-2.55 (m, 1H) 2.38-2.24 (m, 3H) 2.12-1.99 (m, 2H) 1.54- | |
| | 1.47 (m, 3H) | |
| 207 | 1H NMR (400 MHz, METHANOL-d4) δ: 7.17 (d, J = 5.6 Hz, 1H) 6.14 | MS (ESI): m/z = |
| | (t, J = 5.6 Hz, 1H) 6.06-6.01 (m, 2H) 6.00 (s, 1H) 3.94 (d, J = 6.1 Hz, 2H) 3.49 | 372.1 [M + H] |
| | (br. s, 2H) 2.95-2.87 (m, 3H) 2.78 (br. s, 2H) 2.40-2.27 (m, 3H) 2.07 (br. s, | |
| | 2H) | |
| 208 | 1H NMR (400 MHz, METHANOL-d4) δ: 8.46 (s, 1 H) 7.16 (d, J = 6.50 Hz, | MS (ESI): m/z = |
| | 1 H) 6.07 (t, J = 6.63 Hz, 1 H) 5.99 (s, 2 H) 5.79 (d, J = 5.88 Hz, 1 H) 4.00 | 403.1 [M + H] |
| | (d, J = 6.63 Hz, 2 H) 3.46-3.58 (m, 2 H) 2.89 (s, 3 H) 2.79 (s, 5 H) 2.26 | |
| | (d, J = 3.25 Hz, 3 H) 2.04-2.13 (m, 2 H) | |
| 209 | 1H NMR (400 MHz, METHANOL-d4) δ: 7.16 (d, J = 6.3 Hz, 1H) 6.14 (s, | MS (ESI): m/z = |
| | 1H) 6.04 (s, 2H) 3.94 (d, J = 6.4 Hz, 2H) 3.51-3.46 (m, 2H) 2.98 (s, 3H) | 390.1 [M + H] |
| | 2.82-2.77 (m, 2H) 2.32 (d, J = 2.9 Hz, 3H) 2.11-2.05 (m, 2H) | |
| 210 | 1H NMR (400 MHz, METHANOL-d4) δ: 7.42 (d, J = 7.2 Hz, 1H) 5.98 (t, J = | MS (ESI): m/z = |
| | 6.5 Hz, 1H) 4.65 (br. s, 2H) 4.14-3.96 (m, 2H) 3.75-3.65 (m, 1H) 3.51- | 412.1 [M + H] |
| | 3.42 (m, 1H) 3.25 (br t, J = 8.6 Hz, 2H) 2.93 (brd, J = 3.5 Hz, 6H) 2.81- | |
| | 2.69 (m, 2H) 2.49 (q, J = 7.2 Hz, 2H) 2.34 (s, 3H) 2.11 (br. s, 2H) 1.09 (t, J = | |
| | 7.4 Hz, 3H) | |
| 211 | 1H NMR (400 MHz, METHANOL-d4) δ: 6.87 (d, J = 6.38 Hz, 1 H) 6.15 | MS (ESI): m/z = |
| | (t, J = 6.32 Hz, 1 H) 6.07 (s, 2 H) 5.70 (d, J = 6.75 Hz, 1 H) 3.95 (d, J = 6.38 Hz, | 389.0 [M + H] |
| | 2 H) 3.42-3.55 (m, 2 H) 2.86 (s, 3 H) 2.77-2.82 (m, 2 H) 2.36 (d, J = 3.13 | |
| | Hz, 3 H) 2.03-2.12 (m, 2 H) | |
| 212 | 1H NMR (400 MHz, METHANOL-d4) δ: 7.44 (d, J = 7.5 Hz, 1H) 6.05 (t, J = | MS (ESI): m/z = |
| | 6.3 Hz, 1H) 4.67 (t, J = 8.7 Hz, 2H) 3.94 (d, J = 6.4 Hz, 2H) 3.53-3.48 (m, | 398.1 [M + H] |
| | 2H) 3.26 (t, J = 8.8 Hz, 2H) 2.94 (s, 3H) 2.78-2.74 (m, 2H) 2.51 (d, J = 7.6 | |
| | Hz, 2H) 2.36 (s, 3H) 2.13-2.06 (m, 2H) 1.11 (t, J = 7.5 Hz, 3H) | |
| 214 | 1H NMR (400 MHz, METHANOL-d4) δ: 7.43 (d, J = 7.50 Hz, 1 H) 5.98 (t, J = | MS (ESI): m/z = |
| | 6.63 Hz, 1 H) 4.62-4.69 (m, 2 H) 3.93-4.17 (m, 2 H) 3.68 (d, J = 4.25 | 398.1 [M + H] |
| | Hz, 1 H) 3.38-3.52 (m, 1 H) 3.25 (t, J = 8.69 Hz, 2 H) 2.93 (s, 6 H) 2.67- | |
| | 2.82 (m, 2 H) 2.33 (s, 3 H) 2.11 (br s, 2 H) 1.99 (s, 3 H) | |
| 215 | 1H NMR (400 MHz, METHANOL-d4) δ: 7.40 (d, J = 7.4 Hz, 1H) 6.00 | MS (ESI): m/z = |
| | (t, J = 6.6 Hz, 1H) 4.68 (br t, J = 8.7 Hz, 2H) 4.14-3.99 (m, 2H) 3.70 (br. s, | 418.1 [M + H] |
| | 1H) 3.48 (br. s, 1H) 3.27 (br t, J = 8.7 Hz, 2H) 2.97 (s, 3H) 2.95 (s, 3H) 2.81- | |
| | 2.72 (m, 2H) 2.47 (s, 3H) 2.13 (br. s, 2H) | |
| 216 | 1H NMR (400 MHz, METHANOL-d4) δ: 7.34-7.22 (m, 1H) 6.90-6.78 (m, | MS (ESI): m/z = |
| | 1H) 5.99-5.94 (m, 1H) 5.93-5.86 (m, 1H) 4.33-4.23 (m, 4H) 3.94-3.82 | 372.1 [M + H] |
| | (m, 2H) 3.52-3.43 (m, 2H) 2.82-2.70 (m, 2H) 2.39-2.23 (m, 2H) 2.11- | |
| | 1.99 (m, 2H) | |
| 217 | 1H NMR (400 MHz, METHANOL-d4) δ: 8.53 (s, 2H) 7.55-7.47 (m, 1H) | MS (ESI): m/z = |
| | 7.51 (br d, J = 6.9 Hz, 1H) 5.98 (br s, 1H) 5.92 (br s, 1H) 4.65 (brt, J = 8.6 | 383.1 [M + H] |
| | Hz, 2H) 4.02 (br d, J = 5.8 Hz, 2H) 3.56 (br s, 2H) 3.33 (br s, 2H) 3.25 (br | |
| | t, J = 8.6 Hz, 2H) 2.92 (s, 3H) 2.76 (br d, J = 8.9 Hz, 2H) 2.27 (s, 3H) 2.11 | |
| | (br s, 2H) | |
| 218 | 1H NMR (400 MHz, METHANOL-d4) δ: 7.38 (s, 1 H) 7.11-7.21 (m, 1 H) | MS (ESI): m/z = |
| | 6.05 (br d, J = 3.29 Hz, 1 H) 4.59-4.66 (m, 2 H) 4.04-4.15 (m, 1 H) 3.88- | 385.1 [M + H] |
| | 4.03 (m, 1 H) 3.63-3.74 (m, 1 H) 3.36-3.54 (m, 1 H) 3.25 (br t, J = 8.44 | |
| | Hz, 2 H) 3.02 (s, 3 H) 2.92 (s, 3 H) 2.82-2.88 (m, 2 H) 2.30 (br s, 2 H) 2.00- | |
| | 2.17 (m, 2 H) | |
| 219 | 1H NMR (400 MHz, METHANOL-d4) δ: 7.36 (d, J = 7.3 Hz, 1H) 5.98 (br | MS (ESI): m/z = |
| | t, J = 6.3 Hz, 1H) 4.66 (br t, J = 8.8 Hz, 2H) 4.16-4.06 (m, 1H) 4.04-3.93 | 406.2 [M + H] |
| | (m, 1H) 3.68 (br d, J = 3.1 Hz, 1H) 3.51-3.41 (m, 1H) 3.25 (t, J = 8.8 Hz, | |
| | 2H) 2.93 (s, 3H) 2.81-2.69 (m, 2H) 2.33-2.29 (m, 2H) 2.11 (br s, 2H) | |
| 220 | 1H NMR (400 MHz, METHANOL-d4) δ: 7.32-7.41 (m, 1 H) 7.11-7.21 | MS (ESI): m/z = |
| | (m, 1 H) 6.05 (t, J = 6.75 Hz, 1 H) 4.62 (br t, J = 8.69 Hz, 2 H) 3.86-4.17 (m, | 388.1 [M + H] |
| | 2 H) 3.58-3.75 (m, 1 H) 3.35-3.52 (m, 1 H) 3.25 (br t, J = 8.76 Hz, 2 H) | |
| | 2.90-2.97 (m, 3 H) 2.86 (br d, J = 4.25 Hz, 2 H) 2.27-2.35 (m, 2 H) 1.87- | |
| | 2.26 (m, 2 H) | |
| 221 | 1H NMR (400 MHz, METHANOL-d4) δ: 7.35 (d, J = 7.9 Hz, 1H) 5.98 | MS (ESI): m/z = |
| | (t, J = 6.4 Hz, 1H) 4.66 (dt, J = 2.5, 8.8 Hz, 2H) 4.15-4.07 (m, 1H) 4.02- | 403.1 [M + H] |
| | 3.95 (m, 1H) 3.76-3.64 (m, 1H) 3.52-3.39 (m, 1H) 3.25 (t, J = 8.8 Hz, 2H) | |
| | 2.96 (s, 3H) 2.93 (s, 3H) 2.81-2.70 (m, 2H) 2.32-2.29 (m, 2H) 2.15-2.05 | |
| | (m, 2H) | |
| 222 | 1H NMR (400 MHz, METHANOL-d4) δ: 7.33 (d, J = 7.5 Hz, 1H) 5.99 | MS (ESI): m/z = |
| | (t, J = 6.7 Hz, 1H) 4.68 (br t, J = 8.6 Hz, 2H) 4.13-3.99 (m, 2H) 3.70 (br s, | 420.2 [M + H] |
| | 1H) 3.49-3.42 (m, 3H) 3.26 (br t, J = 8.8 Hz, 2H) 2.95 (s, 3H) 2.76 (br d, J = | |
| | 7.6 Hz, 2H) 2.33 (br d, J = 1.6 Hz, 2H) 2.13 (s, 2H) | |
| 224 | 1H NMR (400 MHz, METHANOL-d4) δ: 7.36 (d, J = 7.5 Hz, 1H) 6.03 | MS (ESI): m/z = |
| | (t, J = 6.4 Hz, 1H) 4.65 (t, J = 8.8 Hz, 2H) 3.92 (d, J = 6.5 Hz, 2H) 3.51-3.45 | 404.0 [M + H] |
| | (m, 2H) 3.24 (t, J = 8.7 Hz, 2H) 2.95 (s, 3H) 2.76-2.71 (m, 2H) 2.45 (s, 3H) | |
| | 2.10-2.03 (m, 2H) | |
| 225 | 1H NMR (400 MHz, METHANOL-d4) δ: 7.34 (d, J = 7.4 Hz, 1H) 6.03 | MS (ESI): m/z = |
| | (t, J = 6.4 Hz, 1H) 4.66 (t, J = 8.8 Hz, 2H) 3.92 (d, J = 6.4 Hz, 2H) 3.51-3.44 | 392.1 [M + H] |
| | (m, 2H) 3.25 (br t, J = 8.7 Hz, 2H) 2.77-2.71 (m, 2H) 2.33-2.29 (m, 2H) | |
| | 2.11-2.04 (m, 2H) | |
| 226 | 1H NMR (400 MHz, METHANOL-d4) δ: 7.31 (br d, J = 7.1 Hz, 1H) 6.06- | MS (ESI): m/z = |
| | 5.98 (m, 1H) 4.66 (br t, J = 8.6 Hz, 2H) 3.92 (br d, J = 6.0 Hz, 2H) 3.51- | 406.1 [M + H] |
| | 3.41 (m, 4H) 3.28-3.21 (m, 2H) 2.74 (br d, J = 4.1 Hz, 2H) 2.31 (br s, 2H) | |
| | 2.07 (br s, 2H) | |
| 227 | 1H NMR (400 MHz, METHANOL-d4) δ: 7.39 (br d, J = 7.13 Hz, 1 H) 5.92- | MS (ESI): m/z = |
| | 6.04 (m, 2 H) 4.67 (br t, J = 8.19 Hz, 2 H) 4.09 (br d, J = 5.50 Hz, 1 H) 4.00 | 387.1 [M + H] |
| | (br d, J = 7.25 Hz, 1 H) 3.62-3.74 (m, 1 H) 3.40-3.49 (m, 1 H) 3.25 (br t, J = | |
| | 8.63 Hz, 2 H) 2.88-2.98 (m, 3 H) 2.68-2.82 (m, 2 H) 2.28 (s, 3 H) 2.10 | |
| | (br d, J = 8.76 Hz, 2 H) | |
| 228 | 1H NMR (400 MHz, METHANOL-d4) δ: 7.34 (d, J = 7.5 Hz, 1H) 5.98 (br | MS (ESI): m/z = |
| | t, J = 6.2 Hz, 1H) 4.66 (br t, J = 8.9 Hz, 2H) 4.20-3.89 (m, 2H) 3.78-3.36 | 405.1 [M + H] |
| | (m, 2H) 3.25 (br t, J = 8.8 Hz, 2H) 2.93 (s, 3H) 2.83-2.68 (m, 2H) 2.32 | |
| | (d, J = 2.4 Hz, 3H) 2.17-2.04 (m, 2H) | |
| 229 | 1H NMR (400 MHz, METHANOL-d4) δ: 7.32-7.42 (m, 1 H) 7.12-7.18 | MS (ESI): m/z = |
| | (m, 1 H) 6.06-6.20 (m, 1 H) 4.61 (t, J = 8.77 Hz, 2 H) 3.90 (d, J = 6.58 Hz, 2 | 371.1 [M + H] |
| | H) 3.36-3.57 (m, 2 H) 3.25 (t, J = 8.44 Hz, 2 H) 3.02 (s, 3 H) 2.82-2.92 (m, | |
| | 2 H) 2.28-2.32 (m, 2 H) 1.87-2.20 (m, 2 H) | |
| 230 | 1H NMR (400 MHz, METHANOL-d4) δ: 7.34-7.24 (m, 1H) 6.91-6.81 (m, | MS (ESI): m/z = |
| | 1H) 6.00-5.94 (m, 1H) 5.89-5.79 (m, 1H) 4.32-4.25 (m, 4H) 4.13-3.89 | 392.1 [M + H] |
| | (m, 2H) 3.76-3.58 (m, 1H) 3.50-3.39 (m, 1H) 2.96-2.89 (m, 3H) 2.82- | |
| | 2.71 (m, 2H) 2.38-2.32 (m, 1H) 2.31-2.24 (m, 2H) 2.15-2.04 (m, 2H) | |
| 231 | 1H NMR (400 MHz, METHANOL-d4) δ: 8.50 (s, 2H) 7.45 (br d, J = 6.8 Hz, | MS (ESI): m/z = |
| | 1H) 6.02 (br s, 1H) 5.91 (br s, 1H) 4.63 (br t, J = 8.7 Hz, 2H) 3.91 (br d, J = | 369.1 [M + H] |
| | 4.3 Hz, 2H) 3.47 (br s, 2H) 3.31 (br s, 2H) 3.22 (br t, J = 8.6 Hz, 2H) 2.77- | |
| | 2.71 (m, 2H) 2.26 (s, 3H) 2.07 (br s, 2H) | |
| 232 | 1H NMR (400 MHz, METHANOL-d4) δ: 7.37 (s, 1 H) 7.16 (s, 1 H) 6.06- | MS (ESI): m/z = |
| | 6.14 (m, 1 H) 4.62 (t, J = 8.66 Hz, 2 H) 3.90 (br d, J = 6.36 Hz, 2 H) 3.42-3.52 | 374.1 [M + H] |
| | (m, 2 H) 3.25 (t, J = 8.66 Hz, 2 H) 2.82-2.94 (m, 2 H) 2.28-2.34 (m, 2 H) | |
| | 2.00-2.11 (m, 2 H) | |
| 233 | 1H NMR (400 MHz, METHANOL-d4) δ: 7.12 (s, 1 H) 6.90 (d, J = 2.13 Hz, | MS (ESI): m/z = |
| | 1 H) 6.22 (t, J = 6.50 Hz, 1 H) 6.03 (s, 2 H) 3.93 (d, J = 6.63 Hz, 2 H) 3.47- | 390.1 [M + H] |
| | 3.51 (m, 4 H) 2.87-2.91 (m, 2 H) 2.29 (br d, J = 1.75 Hz, 2 H) 2.02-2.09 | |
| | (m, 2 H). | |
| 234 | 1H NMR (400 MHz, METHANOL-d4) δ: 7.39 (br d, J = 7.2 Hz, 1H) 6.03- | MS (ESI): m/z = |
| | 5.94 (m, 2H) 4.70-4.61 (m, 2H) 4.24-3.81 (m, 2H) 3.80-3.58 (m, 1H) 3.58- | 388.1 [M + H] |
| | 3.34 (m, 1H) 3.25 (br t, J = 8.8 Hz, 2H) 2.93 (s, 3H) 2.81-2.68 (m, 2H) | |
| | 2.38-2.25 (m, 2H) 2.11 (td, J = 1.6, 2.8 Hz, 2H) | |
| 235 | 1H NMR (400 MHz, METHANOL-d4) δ: 7.33 (d, J = 7.5 Hz, 1H) 6.04 | MS (ESI): m/z = |
| | (t, J = 6.3 Hz, 1H) 4.66 (t, J = 8.8 Hz, 2H) 3.92 (d, J = 6.4 Hz, 2H) 3.50-3.45 | 389.1 [M + H] |
| | (m, 2H) 3.25 (t, J = 8.8 Hz, 2H) 2.96 (s, 3H) 2.77-2.71 (m, 2H) 2.33-2.30 | |
| | (m, 2H) 2.11-2.04 (m, 2H) | |
| 236 | 1H NMR (400 MHz, METHANOL-d4) δ: 7.07 (d, J = 8.63 Hz, 1 H) 5.81 (t, J = | MS (ESI): m/z = |
| | 6.57 Hz, 1 H) 5.64 (d, J = 6.75 Hz, 1 H) 4.19-4.30 (m, 2 H) 3.93-4.15 | 415.1 [M + H] |
| | (m, 2 H) 3.69 (br d, J = 4.88 Hz, 1 H) 3.47 (br d, J = 6.00 Hz, 1 H) 2.93 (s, 3 | |
| | H) 2.84 (s, 3 H) 2.81 (br t, J = 6.50 Hz, 2 H) 2.68 (br dd, J = 17.26, 4.13 Hz, | |
| | 2 H) 2.35 (d, J = 3.13 Hz, 3 H) 2.12 (br d, J = 4.50 Hz, 2 H) 1.97-2.06 (m, 2 | |
| | H) | |
| 237 | 1H NMR (400 MHz, METHANOL-d4) δ: 7.36 (s, 1H) 7.13 (br s, 1H) 6.14- | MS (ESI): m/z = |
| | 6.05 (m, 1H) 4.62 (t, J = 8.7 Hz, 2H) 3.90 (br d, J = 5.8 Hz, 2H) 3.52-3.44 | 388.1 [M + H] |
| | (m, 4H) 3.24 (t, J = 8.6 Hz, 2H) 2.88-2.81 (m, 2H) 2.30 (br s, 2H) 2.05 (br | |
| | d, J = 4.5 Hz, 2H) | |
| 238 | 1H NMR (400 MHz, METHANOL-d4) δ: 7.47-7.32 (m, 1H) 6.05-5.91 (m, | MS (ESI): m/z = |
| | 2H) 4.71-4.61 (m, 2H) 4.19-3.90 (m, 2H) 3.77-3.36 (m, 2H) 3.25 (br t, J = | 387.1 [M + H] |
| | 8.6 Hz, 2H) 2.93 (s, 3H) 2.81-2.69 (m, 2H) 2.28 (s, 3H) 2.15-2.05 (m, 2H) | |
| | 1H NMR (400 MHz, METHANOL-d4) δ: 7.37-7.42 (m, 1 H) 5.94-6.02 | |
| | (m, 2 H) 4.61-4.71 (m, 2 H) 3.95-4.15 (m, 2 H) 3.68 (br s, 1 H) 3.41-3.51 | |
| | (m, 1 H) 3.25 (br t, J = 8.76 Hz, 2 H) 2.93 (s, 3 H) 2.74 (br d, J = 7.50 Hz, 2 | |
| | H) 2.28 (s, 3 H) 2.10 (br d, J = 8.63 Hz, 2 H) | |
| 239 | 1H NMR (400 MHz, METHANOL-d4) δ: 7.31 (d, J = 7.5 Hz, 1H) 6.03 | MS (ESI): m/z = |
| | (t, J = 6.4 Hz, 1H) 4.66 (t, J = 8.8 Hz, 2H) 3.92 (d, J = 6.4 Hz, 2H) 3.50-3.41 | 402.1 [M + H] |
| | (m, 4H) 3.31 (s, 4H) 3.24 (t, J = 8.7 Hz, 2H) 2.77-2.71 (m, 2H) 2.32 (d, J = | |
| | 2.9 Hz, 3H) 2.11-2.03 (m, 2H) 1.18 (t, J = 7.2 Hz, 3H) | |
| 240 | 1H NMR (400 MHz, METHANOL-d4) δ: 7.22-7.17 (m, 1H) 6.94-6.90 (m, | MS (ESI): m/z = |
| | 1H) 6.25-6.20 (m, 1H) 6.04-6.01 (m, 2H) 3.95-3.90 (m, 2H) 3.50-3.45 | 376.1 [M + H] |
| | (m, 2H) 2.92-2.86 (m, 2H) 2.33-2.29 (m, 2H) 2.09-2.00 (m, 2H) | |
| 241 | 1H NMR (400 MHz, METHANOL-d4) δ: 7.32 (d, J = 7.5 Hz, 1H) 5.98 | MS (ESI): m/z = |
| | (t, J = 6.6 Hz, 1H) 4.67 (br t, J = 8.7 Hz, 2H) 4.15-4.06 (m, 1H) 4.03-3.95 | 416.1 [M + H] |
| | (m, 1H) 3.74-3.65 (m, 1H) 3.44 (q, J = 7.1 Hz, 3H) 3.25 (br t, J = 8.8 Hz, | |
| | 2H) 2.93 (s, 3H) 2.83-2.69 (m, 2H) 2.33 (d, J = 2.9 Hz, 3H) 2.11 (br d, J = | |
| | 3.6 Hz, 2H) 1.18 (t, J = 7.2 Hz, 3H) | |
| 242 | 1H NMR (400 MHz, METHANOL-d4) δ: 7.33 (d, J = 7.5 Hz, 1H) 6.04 | MS (ESI): m/z = |
| | (t, J = 6.5 Hz, 1H) 4.66 (t, J = 8.8 Hz, 2H) 3.92 (d, J = 6.6 Hz, 2H) 3.51-3.45 | 388.1 [M + H] |
| | (m, 2H) 3.25 (t, J = 8.6 Hz, 2H) 2.96 (s, 3H) 2.77-2.71 (m, 2H) 2.32 (d, J = | |
| | 2.9 Hz, 3H) 2.11-2.03 (m, 2H) | |
| 243 | 1H NMR (400 MHz, METHANOL-d4) δ: 7.06 (d, J = 8.63 Hz, 1 H) 5.87 | MS (ESI): m/z = |
| | (t, J = 6.32 Hz, 1 H) 5.64 (d, J = 6.75 Hz, 1 H) 4.17-4.29 (m, 2 H) 3.90 (d, J = | 401.1 [M + H] |
| | 6.38 Hz, 2 H) 3.44-3.53 (m, 2 H) 2.84 (s, 3 H) 2.81 (br t, J = 6.50 Hz, 2 H) | |
| | 2.62-2.70 (m, 2 H) 2.35 (d, J = 3.13 Hz, 3 H) 2.05-2.13 (m, 2 H) 1.97- | |
| | 2.05 (m, 2 H) | |
| 244 | 1H NMR (400 MHz, METHANOL-d4) δ: 7.24 (br s, 1H) 6.97 (br s, 1H) 6.19 | MS (ESI): m/z = |
| | (br t, J = 6.6 Hz, 1H) 6.06-6.03 (m, 2H) 5.97 (s, 1H) 4.21-4.10 (m, 1H) | 385.1 [M + H] |
| | 4.05-3.94 (m, 1H) 3.70 (br d, J = 3.0 Hz, 1H) 3.51-3.43 (m, 3H) 2.95 (s, | |
| | 3H) 2.93-2.89 (m, 2H) 2.31 (s, 3H) 2.18-2.02 (m, 2H) | |
| 245 | 1H NMR (400 MHz, METHANOL-d4) δ: 7.34 (br d, J = 7.5 Hz, 1H) 5.98 (br | MS (ESI): m/z = |
| | t, J = 6.6 Hz, 1H) 4.66 (br t, J = 8.5 Hz, 2H) 4.17-3.93 (m, 2H) 3.68 (br s, | 402.1 [M + H] |
| | 1H) 3.46 (br d, J = 2.3 Hz, 1H) 3.25 (br t, J = 8.7 Hz, 2H) 2.94 (d, J = 8.8 Hz, | |
| | 6H) 2.84-2.66 (m, 2H) 2.33 (d, J = 2.8 Hz, 3H) 2.11 (br s, 2H) | |
| 246 | 1H NMR (400 MHz, METHANOL-d4) δ: 7.26-7.17 (m, 1H) 6.96 (s, 1H) | MS (ESI): m/z = |
| | 6.26 (br t, J = 6.7 Hz, 1H) 6.06 (s, 2H) 5.98 (s, 1H) 3.96 (d, J = 6.5 Hz, 2H) | 371.1 [M + H] |
| | 3.53-3.49 (m, 2H) 3.48 (s, 2H) 2.95-2.89 (m, 2H) 2.32 (s, 3H) 2.12-2.05 | |
| | (m, 2H) | |
| 247 | 1H NMR (400 MHz, METHANOL-d4) δ: 7.19-7.08 (m, 1H) 7.02-6.93 (m, | MS (ESI): m/z = |
| | 1H) 6.51-6.22 (m, 1H) 6.14-6.03 (m, 1H) 5.91-5.59 (m, 1H) 4.69-4.58 | 419.1 [M + H] |
| | (m, 2H) 3.97-3.80 (m, 2H) 3.75-3.56 (m, 2H) 3.51-3.40 (m, 2H) 3.29- | |
| | 3.24 (m, 2H) 2.95-2.71 (m, 5H) 2.11-1.98 (m, 2H) | |
| 248 | 1H NMR (400 MHz, METHANOL-d4) δ: 7.27-7.33 (m, 1 H) 7.11 (br s, 1 | MS (ESI): m/z = |
| | H) 5.95 (s, 1 H) 5.84 (br t, J = 6.19 Hz, 1 H) 4.18-4.23 (m, 2 H) 3.86 (br d, J = | 367.1 [ M + H] |
| | 6.25 Hz, 2 H) 3.44-3.50 (m, 2 H) 2.97 (s, 3 H) 2.83 (br t, J = 6.38 Hz, 2 H) | |
| | 2.70-2.76 (m, 2 H) 2.23-2.29 (m, 2 H) 1.98-2.08 (m, 4 H) | |
| 249 | 1H NMR (400 MHz, METHANOL-d4) δ: 7.37 (br d, J = 7.0 Hz, 1H) 6.04 | MS (ESI): m/z = |
| | (t, J = 6.2 Hz, 1H) 5.98 (s, 1H) 4.69-4.62 (m, 2H) 3.92 (d, J = 6.4 Hz, 2H) | 374.1 [M + H] |
| | 3.50-3.45 (m, 2H) 3.25 (t, J = 8.6 Hz, 2H) 2.77-2.72 (m, 2H) 2.39-2.24 | |
| | (m, 2H) 2.11-2.04 (m, 2H) | |
| 250 | 1H NMR (400 MHz, METHANOL-d4) δ: 7.30 (s, 1H) 7.11 (d, J = 2.4 Hz, | MS (ESI): m/z = |
| | 1H) 5.95 (s, 1H) 5.84 (t, J = 6.5 Hz, 1H) 4.25-4.17 (m, 2H) 3.86 (d, J = 6.5 | 370.2 [M + H] |
| | Hz, 2H) 3.51-3.42 (m, 2H) 2.83 (t, J = 6.5 Hz, 2H) 2.77-2.68 (m, 2H) 2.39- | |
| | 2.20 (m, 2H) 2.11-1.95 (m, 4H) | |
| 251 | 1H NMR (400 MHz, METHANOL-d4) δ: 7.32 (s, 1H) 7.13 (d, J = 1.8 Hz, | MS (ESI): m/z = |
| | 1H) 5.95 (s, 1H) 5.78 (t, J = 6.5 Hz, 1H) 4.24-4.18 (m, 2H) 4.09-3.99 (m, | 384.2 [M + H] |
| | 1H) 3.96-3.88 (m, 1H) 3.72-3.64 (m, 1H) 3.48-3.39 (m, 1H) 2.92 (s, 3H) | |
| | 2.83 (t, J = 6.4 Hz, 2H) 2.76-2.70 (m, 2H) 2.26 (br s, 2H) 2.10 (quin, J = 5.4 | |
| | Hz, 2H) 2.05-1.97 (m, 2H) | |
| 252 | 1H NMR (400 MHz, METHANOL-d4) δ: 7.25 (s, 1H) 6.97 (s, 1H) 6.23 | MS (ESI): m/z = |
| | (t, J = 6.2 Hz, 1H) 6.02 (s, 1H) 6.00 (br s, 1H) 5.97 (s, 1H) 3.93 (d, J = 6.6 | 358.1 [M + H] |
| | Hz, 2H) 3.51-3.44 (m, 2H) 2.92-2.86 (m, 2H) 2.34-2.22 (m, 2H) 2.08- | |
| | 2.01 (m, 2H) | |
| 253 | 1H NMR (400 MHz, METHANOL-d4) δ: 7.38 (br d, J = 7.5 Hz, 1H) 6.04 (br | MS (ESI): m/z = |
| | t, J = 5.9 Hz, 1H) 5.98 (s, 1H) 4.66 (t, J = 8.6 Hz, 2H) 3.92 (br d, J = 6.4 Hz, | 373.1 [M + H] |
| | 2H) 3.50-3.45 (m, 2H) 3.25 (t, J = 8.4 Hz, 2H) 2.77-2.72 (m, 2H) 2.39-2.27 | |
| | (m, 3H) 2.11-2.04 (m, 2H) 1H NMR (400 MHz, METHANOL-d4) δ: 7.35-7.41 | |
| | (m, 1 H) 6.01-6.06 (m, 1 H) 5.98 (s, 1 H) 4.66 (t, J = 8.69 Hz, 2 H) 3.92 (br d, | |
| | J = 6.38 Hz, 2 H) 3.44-3.51 (m, 2 H) 3.25 (br t, J = 8.69 Hz, 2 H) 2.70-2.78 | |
| | (m, 2 H) 2.28 (s, 3 H) 2.03-2.12 (m, 2 H) 1H NMR (400 MHz, | |
| | METHANOL-d4) δ: 7.35-7.40 (m, 1 H) 6.03 (t, J = 6.32 Hz, 1 H) 5.97 | |
| | (d, J = 0.75 Hz, 1 H) 4.65 (t, J = 8.69 Hz, 2 H) 3.92 (d, J = 6.38 Hz, 2 H) | |
| | 3.45-3.50 (m, 2 H) 3.24 (t, J = 8.69 Hz, 2 H) 2.71-2.77 (m, 2 H) 2.28 (s, 3 H) | |
| | 2.04-2.11 (m, 2 H) | |
| 254 | 1H NMR (400 MHz, METHANOL-d4) δ: 7.30-7.20 (m, 1H) 6.97 (s, 1H) | MS (ESI): m/z = |
| | 6.18 (t, J = 6.8 Hz, 1H) 6.01 (s, 2H) 5.99-5.95 (m, 1H) 4.18-4.06 (m, 1H) | 372.1 [M + H] |
| | 4.05-3.92 (m, 1H) 3.76-3.61 (m, 1H) 3.52-3.38 (m, 1H) 2.93 (s, 3H) 2.92- | |
| | 2.85 (m, 2H) 2.40-2.25 (m, 2H) 2.21-1.98 (m, 2H) | |
| 255 | 1H NMR (400 MHz, METHANOL-d4) δ: 7.20 (d, J = 7.6 Hz, 1H) 5.99 | MS (ESI): m/z = |
| | (t, J = 6.6 Hz, 1H) 5.64 (d, J = 6.6 Hz, 1H) 4.68 (br t, J = 8.6 Hz, 2H) 4.16- | 401.1 [M + H] |
| | 3.95 (m, 2H) 3.68 (br s, 1H) 3.46 (br s, 1H) 3.26 (br t, J = 8.7 Hz, 2H) 2.93 | |
| | (s, 3H) 2.84 (s, 3H) 2.74 (br d, J = 6.8 Hz, 2H) 2.35 (d, J = 3.1 Hz, 3H) 2.11 | |
| | (br s, 2H) | |
| 256 | 1H NMR (400 MHz, METHANOL-d4) δ: 7.33 (d, J = 7.2 Hz, 1H) 6.03 | MS (ESI): m/z = |
| | (t, J = 6.2 Hz, 1H) 4.65 (t, J = 8.8 Hz, 2H) 3.91 (d, J = 6.1 Hz, 2H) 3.52-3.43 | 391.1 [M + H] |
| | (m, 2H) 3.24 (t, J = 8.8 Hz, 2H) 2.78-2.69 (m, 2H) 2.32 (d, J = 2.9 Hz, 3H) | |
| | 2.11-2.03 (m, 2H) | |
| 257 | 1H NMR (400 MHz, METHANOL-d4) δ: 7.22 (d, J = 7.5 Hz, 1H) 6.08 | MS (ESI): m/z = |
| | (t, J = 6.4 Hz, 1H) 5.67 (br d, J = 6.7 Hz, 1H) 4.70 (t, J = 8.7 Hz, 2H) 3.95 | 387.1 [M + H] |
| | (d, J = 6.5 Hz, 2H) 3.54-3.48 (m, 2H) 3.29 (br t, J = 8.6 Hz, 2H) 2.87 (s, 3H) | |
| | 2.80-2.74 (m, 2H) 2.38 (d, J = 3.2 Hz, 3H) 2.14-2.07 (m, 2H) | |
| 258 | 1H NMR (400 MHz, METHANOL-d4) δ: 7.37 (br d, J = 7.3 Hz, 1H) 6.07 | MS (ESI): m/z = |
| | (t, J = 6.4 Hz, 1H) 4.69 (t, J = 8.7 Hz, 2H) 3.96 (d, J = 6.4 Hz, 2H) 3.54-3.50 | 405.1 [M + H] |
| | (m, 2H) 3.46 (s, 2H) 3.28 (br t, J = 8.7 Hz, 2H) 2.79-2.75 (m, 2H) 2.36 | |
| | (d, J = 2.7 Hz, 3H) 2.14-2.06 (m, 2H) | |
| 259 | 1H NMR (400 MHz, METHANOL-d4) δ: 7.29 (s, 1H) 7.09 (d, J = 2.4 Hz, | MS (ESI): m/z = |
| | 1H) 5.93 (s, 1H) 5.78 (d, J = 6.8 Hz, 1H) 4.31-4.16 (m, 2H) 4.11-3.84 (m, | 397.2 [M + H] |
| | 2H) 3.50-3.38 (m, 3H) 2.92 (s, 3H) 2.85-2.82 (m, 2H) 2.37-2.25 (m, 2H) | |
| | 2.28 (s, 3H) 2.16-2.09 (m, 2H) 2.05-1.98 (m, 2H) | |
| 260 | 1H NMR (400 MHz, METHANOL-d4) δ: 7.40 (br d, J = 8.3 Hz, 1H) 6.03- | MS (ESI): m/z = |
| | 5.98 (m, 2H) 4.73-4.67 (m, 2H) 4.19-4.10 (m, 1H) 4.06-3.98 (m, 1H) 3.78- | 402.2 [M + H] |
| | 3.69 (m, 1H) 3.54-3.45 (m, 1H) 3.38 (s, 2H) 3.29 (br t, J = 8.7 Hz, 2H) | |
| | 2.97 (s, 3H) 2.83-2.72 (m, 2H) 2.32-2.26 (m, 2H) 2.18-2.09 (m, 2H) | |
| 261 | 1H NMR (400 MHz, METHANOL-d4) δ: 7.35 (br d, J = 7.5 Hz, 1H) 5.99 | MS (ESI): m/z = |
| | (t, J = 6.7 Hz, 1H) 4.68 (br t, J = 8.6 Hz, 2H) 4.16-4.08 (m, 1H) 4.05-3.96 | 419.2 [M + H] |
| | (m, 1H) 3.70 (br d, J = 4.3 Hz, 1H) 3.52-3.46 (m, 1H) 3.44 (s, 2H) 3.27 (br | |
| | t, J = 8.8 Hz, 2H) 2.95 (s, 3H) 2.84-2.71 (m, 2H) 2.34 (d, J = 2.9 Hz, 3H) | |
| | 2.13 (br s, 2H) | |
| 262 | 1H NMR (400 MHz, METHANOL-d4) δ: 7.38 (br d, J = 7.58 Hz, 1 H) 6.07 | MS (ESI): m/z = |
| | (t, J = 6.11 Hz, 1 H) 5.99 (s, 1 H) 4.67-4.72 (m, 2 H) 3.95 (d, J = 6.36 Hz, 2 | 388.2 [M + H] |
| | H) 3.50-3.53 (m, 2 H) 3.38 (br s, 2 H) 3.28 (br t, J = 8.62 Hz, 2 H) 2.76- | |
| | 2.79 (m, 2 H) 2.30 (br s, 2 H) 2.08-2.13 (m, 2 H) | |
| 263 | 1H NMR (400 MHz, METHANOL-d4) δ: 7.29-7.24 (m, 1H) 7.11-7.06 (m, | MS (ESI): m/z = |
| | 1H) 5.93 (s, 1H) 5.84 (t, J = 6.4 Hz, 1H) 4.26-4.17 (m, 2H) 3.90-3.82 (m, | 383.2 [M + H] |
| | 2H) 3.51-3.41 (m, 4H) 2.88-2.78 (m, 2H) 2.77-2.67 (m, 2H) 2.28 (s, 3H) | |
| | 2.13-1.95 (m, 4H) | |
| 264 | 1H NMR (400 MHz, METHANOL-d4) δ: 7.30 (br s, 1H) 7.12 (d, J = 1.7 Hz, | MS (ESI): m/z = |
| | 1H) 5.95 (s, 1H) 5.84 (t, J = 6.4 Hz, 1H) 4.24-4.18 (m, 2H) 3.86 (d, J = 6.5 | 369.1 [M + H] |
| | Hz, 2H) 3.49-3.44 (m, 2H) 2.83 (t, J = 6.4 Hz, 2H) 2.75-2.69 (m, 2H) 2.37 | |
| | (s, 1H) 2.28 (s, 3H) 2.10-1.96 (m, 4H) | |
| 265 | 1H NMR (400 MHz, METHANOL-d4) δ: 7.26 (s, 1 H) 6.98 (br s, 1 H) 6.17 | MS (ESI): m/z = |
| | (br d, J = 9.51 Hz, 1 H) 5.97 (s, 1 H) 6.03 (s, 2 H) 3.95-4.18 (m, 2 H) 3.43- | 369.1 [M + H] |
| | 3.73 (m, 2 H) 2.98 (s, 3 H) 2.93 (s, 3 H) 2.90 (br d, J = 5.13 Hz, 2 H) 2.27 (br | |
| | s, 2 H) 2.00-2.17 (m, 2 H) | |
| 266 | 1H NMR (400 MHz, METHANOL-d4) δ: 7.29 (br d, J = 2.25 Hz, 1 H) 6.86 | MS (ESI): m/z = |
| | (d, J = 2.50 Hz, 1 H) 5.96 (s, 1 H) 5.84 (t, J = 6.69 Hz, 1 H) 4.28 (s, 4 H) 4.04 | 383.1 [M + H] |
| | (br d, J = 4.75 Hz, 1 H) 3.95 (br d, J = 6.75 Hz, 1 H) 3.67 (br s, 1 H) 3.44 (br | |
| | s, 1 H) 2.99 (s, 3 H) 2.93 (s, 3 H) 2.75 (br d, J = 5.00 Hz, 2 H) 2.27 (br s, 2 | |
| | H) 2.11 (br s, 2 H) | |
| 267 | 1H NMR (400 MHz, METHANOL-d4) δ: 7.36-7.43 (m, 1 H) 7.13-7.22 | MS (ESI): m/z = |
| | (m, 1 H) 6.01-6.09 (m, 1 H) 4.56-4.68 (m, 2 H) 3.83-4.19 (m, 2 H) 3.38- | 384.1 [M + H] |
| | 3.72 (m, 2 H) 3.20-3.29 (m, 2 H) 2.97-3.07 (m, 3 H) 2.96 (br s, 3 H) 2.75- | |
| | 2.89 (m, 2 H) 2.21-2.41 (m, 3 H) 1.94-2.21 (m, 2 H) | |
| 268 | 1H NMR (400 MHz, METHANOL-d4) δ: 7.31 (s, 1H) 7.13 (br s, 1H) 5.95 | MS (ESI): m/z = |
| | (s, 1H) 5.78 (br t, J = 6.3 Hz, 1H) 4.22 (br s, 2H) 4.07-3.98 (m, 1H) 3.94 (br | 383.2 [M + H] |
| | d, J = 7.0 Hz, 1H) 3.68 (br d, J = 11.5 Hz, 1H) 3.44 (br d, J = 6.5 Hz, 1H) | |
| | 2.92 (s, 3H) 2.83 (br t, J = 6.4 Hz, 2H) 2.72 (br s, 2H) 2.28 (s, 3H) 2.14-1.97 | |
| | (m, 4H) | |
| 269 | 1H NMR (400 MHz, METHANOL-d4) δ: 7.23 (s, 1 H) 6.96 (d, J = 1.75 Hz, | MS (ESI): m/z = |
| | 1 H) 6.23 (t, J = 6.44 Hz, 1 H) 6.02 (s, 2 H) 5.97 (s, 1 H) 3.93 (d, J = 6.38 Hz, | 355.1 [M + H] |
| | 2 H) 3.46-3.50 (m, 2 H) 2.98 (s, 3 H) 2.87-2.91 (m, 2 H) 2.26-2.29 (m, 2 | |
| | H) 2.02-2.08 (m, 2 H) | |
| 270 | 1H NMR (400 MHz, METHANOL-d4) δ: 7.28 (s, 1 H) 6.85 (d, J = 2.00 Hz, | MS (ESI): m/z = |
| | 1 H) 5.96 (s, 1 H) 5.90 (t, J = 6.32 Hz, 1 H) 4.27 (s, 4 H) 3.87 (d, J = 6.38 Hz, | 369.1 [M + H] |
| | 2 H) 3.45-3.50 (m, 2 H) 2.99 (s, 3 H) 2.73-2.79 (m, 2 H) 2.24-2.30 (m, 2 | |
| | H) 2.02-2.09 (m, 2 H) | |
| 271 | 1H NMR (400 MHz, METHANOL-d4) δ: 7.38 (s, 1H) 7.16 (br s, 1H) 6.06 | MS (ESI): m/z = |
| | (br t, J = 6.1 Hz, 1H) 4.62 (br t, J = 8.6 Hz, 2H) 4.14-3.89 (m, 2H) 3.73- | 387.1 [M + H] |
| | 3.62 (m, 1H) 3.49-3.39 (m, 1H) 3.25 (br t, J = 8.6 Hz, 2H) 2.98-2.82 (m, | |
| | 5H) 2.32 (br d, J = 2.3 Hz, 3H) 2.19-1.97 (m, 2H) | |
| 272 | 1H NMR (400 MHz, METHANOL-d4) δ: 7.26 (br s, 1 H) 6.85 (br s, 1 H) | MS (ESI): m/z = |
| | 5.94 (s, 1 H) 5.84 (br t, J = 6.63 Hz, 1 H) 4.28 (s, 4 H) 4.02-4.09 (m, 1 H) | 399.3 [M + H] |
| | 3.95 (br d, J = 6.88 Hz, 1 H) 3.67 (br s, 1 H) 3.45 (s, 3 H) 2.93 (s, 3 H) 2.76 (br | |
| | d, J = 5.63 Hz, 2 H) 2.28 (s, 3 H) 2.11 (br s, 2 H) | |
| 273 | 1H NMR (400 MHz, METHANOL-d4) δ: 7.28-7.23 (m, 1H) 7.00-6.97 (m, | MS (ESI): m/z = |
| | 1H) 6.20-6.15 (m, 1H) 6.03-6.01 (m, 2H) 5.99-5.94 (m, 1H) 4.16-3.94 | 371.1 [M + H] |
| | (m, 2H) 3.72-3.65 (m, 1H) 3.49-3.40 (m, 1H) 2.93-2.92 (m, 3H) 2.91- | |
| | 2.88 (m, 2H) 2.30-2.27 (m, 3H) 2.16-1.98 (m, 2H) | |
| 274 | 1H NMR (400 MHz, METHANOL-d4) δ: 7.36 (br s, 1 H) 7.17 (br s, 1 H) | MS (ESI): m/z = |
| | 6.05 (t, J = 6.88 Hz, 1 H) 5.95 (s, 1 H) 4.62 (br s, 2 H) 4.02-4.10 (m, 1 H) | 369.1 [M + H] |
| | 3.89-3.97 (m, 1 H) 3.62-3.70 (m, 1 H) 3.45-3.49 (m, 1 H) 3.21-3.26 (m, | |
| | 2 H) 2.94 (s, 3 H) 2.85 (br d, J = 2.13 Hz, 2 H) 2.27 (s, 3 H) 2.02-2.12 (m, 2 H) | |
| | 1H NMR (400 MHz, METHANOL-d4) δ: 7.39-7.45 (m, 1 H) 7.20 (s, 1 H) | |
| | 6.06 (t, J = 6.75 Hz, 1 H) 5.96 (d, J = 0.75 Hz, 1 H) 4.60-4.65 (m, 2 H) 4.06- | |
| | 4.13 (m, 1 H) 3.90-3.98 (m, 1 H) 3.64-3.72 (m, 1 H) 3.40-3.47 (m, 1 H) | |
| | 3.26 (br t, J = 8.69 Hz, 2 H) 2.96 (s, 3 H) 2.86 (br d, J = 5.25 Hz, 2 H) 2.28 | |
| | (s, 3 H) 2.01-2.15 (m, 2 H) | |
| 275 | 1H NMR (400 MHz, METHANOL-d4) δ: 7.20 (d, J = 1.75 Hz, 1 H) 6.93 | MS (ESI): m/z = |
| | (d, J = 1.88 Hz, 1 H) 6.22 (t, J = 6.38 Hz, 1 H) 6.02 (s, 2 H) 3.93 (d, J = 6.38 | 375.1 [M + H] |
| | Hz, 2 H) 3.45-3.50 (m, 2 H) 2.85-2.94 (m, 2 H) 2.33 (d, J = 2.88 Hz, 3 H) | |
| | 2.01-2.10 (m, 2 H) | |
| 276 | 1H NMR (400 MHz, METHANOL-d4) δ: 7.37 (s, 1H) 7.22-7.07 (m, 1H) | MS (ESI): m/z = |
| | 6.11 (br d, J = 5.5 Hz, 1H) 4.61 (br t, J = 8.8 Hz, 2H) 3.98-3.82 (m, 2H) | 373.1 [M + H] |
| | 3.51-3.43 (m, 2H) 3.27-3.21 (m, 2H) 2.85 (dd, J = 3.2, 4.5 Hz, 2H) 2.32 (br | |
| | s, 3H) 2.10-1.99 (m, 2H) | |
| 277 | 1H NMR (400 MHz, METHANOL-d4) δ: 7.38 (s, 1 H) 7.16 (d, J = 1.50 Hz, | MS (ESI): m/z = |
| | 1 H) 6.10 (t, J = 6.50 Hz, 1 H) 4.61 (t, J = 8.69 Hz, 2 H) 3.90 (d, J = 6.50 Hz, | 370.1 [M + H] |
| | 2 H) 3.44-3.49 (m, 2 H) 3.25 (t, J = 8.69 Hz, 2 H) 3.02 (s, 3 H) 2.82-2.88 | |
| | (m, 2 H) 2.32 (d, J = 2.88 Hz, 3 H) 2.01-2.09 (m, 2 H) | |
| 278 | 1H NMR (400 MHz, METHANOL-d4) δ: 7.05 (br s, 1H) 6.87 (br s, 1H) 5.93 | MS (ESI): m/z = |
| | (t, J = 6.7 Hz, 2H) 5.47 (br s, 1H) 4.54 (br t, J = 8.7 Hz, 3H) 3.92 (br d, J = | 365.1 [M + H] |
| | 2.1 Hz, 2H) 3.16 (br t, J = 8.7 Hz, 3H) 2.82 (s, 4H) 2.77-2.72 (m, 3H) 2.65 | |
| | (br s, 3H) 2.31-2.18 (m, 4H) 1.98 (br s, 2H) | |
| 279 | 1H NMR (400 MHz, METHANOL-d4) δ: 7.18 (d, J = 1.9 Hz, 1H) 6.92 | MS (ESI): m/z = |
| | (d, J = 2.0 Hz, 1H) 6.22 (s, 1H) 6.02 (s, 2H) 3.93 (d, J = 6.5 Hz, 2H) 3.50- | 372.1 [M + H] |
| | 3.45 (m, 2H) 3.03 (s, 3H) 2.92-2.86 (m, 2H) 2.32 (d, J = 2.9 Hz, 3H) 2.05 | |
| | (br s, 2H) | |
| 281 | 1H NMR (400 MHz, METHANOL-d4) δ: 7.24-7.20 (m, 1H) 7.26-7.18 (m, | MS (ESI): m/z = |
| | 1H) 6.97-6.92 (m, 1H) 6.26-6.19 (m, 1H) 6.03 (s, 2H) 5.97 (s, 1H) 3.95- | 357.1 [M + H] |
| | 3.90 (m, 2H) 3.50-3.45 (m, 2H) 2.96-2.81 (m, 2H) 2.29 (s, 3H) 2.09-2.01 | |
| | (m, 2H) | |
| 282 | 1H NMR (400 MHz, METHANOL-d4) δ: 7.29 (s, 1H) 6.88 (br s, 1H) 5.99 | MS (ESI): m/z = |
| | (s, 1H) 5.86 (br t, J = 6.5 Hz, 1H) 4.30 (s, 4H) 4.13-3.89 (m, 2H) 3.79-3.64 | 385.1 [M + H] |
| | (m, 1H) 3.47 (br s, 1H) 2.95 (s, 3H) 2.78 (br s, 2H) 2.30 (s, 3H) 2.13 (br s, | |
| | 2H) | |
| 283 | 1H NMR (400 MHz, METHANOL-d4) δ: 7.34-7.22 (m, 1H) 6.90-6.78 (m, | MS (ESI): m/z = |
| | 1H) 5.99-5.94 (m, 1H) 5.93-5.86 (m, 1H) 4.33-4.23 (m, 4H) 3.94-3.82 | 372.1 [M + H] |
| | (m, 2H) 3.52-3.43 (m, 2H) 2.82-2.70 (m, 2H) 2.39-2.23 (m, 2H) 2.11- | |
| | 1.99 (m, 2H) | |
| 284 | 1H NMR (400 MHz, METHANOL-d4) δ: 7.39 (s, 1H) 7.18 (d, J = 1.6 Hz, | MS (ESI): m/z = |
| | 1H) 6.10 (s, 1H) 4.62 (t, J = 8.8 Hz, 2H) 3.90 (d, J = 6.5 Hz, 2H) 3.49-3.45 | 386.1 [M + H] |
| | (m, 2H) 3.25 (t, J = 8.6 Hz, 2H) 3.02 (s, 3H) 2.89-2.80 (m, 2H) 2.43 (s, 3H) | |
| | 2.08-2.01 (m, 2H) | |
| 285 | 1H NMR (400 MHz, METHANOL-d4) δ: 7.21-7.34 (m, 1 H) 6.87 (br d, J = | MS (ESI): m/z = |
| | 2.32 Hz, 1 H) 5.95-6.00 (m, 1 H) 5.93 (br t, J = 6.23 Hz, 1 H) 4.27-4.34 | 385.2 [M + H] |
| | (m, 4 H) 3.91 (br d, J = 6.11 Hz, 2 H) 3.47-3.54 (m, 4 H) 2.79 (br d, J = 4.89 | |
| | Hz, 2 H) 2.31 (s, 3 H) 2.10 (br s, 2 H) | |
| 286 | 1H NMR (400 MHz, METHANOL-d4) δ: 7.44 (s, 1 H) 7.21 (br s, 1 H) 6.05 | MS (ESI): m/z = |
| | (br s, 1 H) 4.56-4.65 (m, 2 H) 4.08 (br s, 1 H) 3.88-4.02 (m, 1 H) 3.67 (br | 380.1 [M + H] |
| | s, 1 H) 3.37-3.51 (m, 1 H) 3.25 (br t, J = 8.69 Hz, 1 H) 3.21-3.20 (m, 1 H) | |
| | 3.01 (s, 3 H) 2.89-2.95 (m, 3 H) 2.85 (br s, 2 H) 2.33 (s, 3 H) 2.01-2.18 (m, | |
| | 2 H) 1.99 (s, 3 H) | |
| 287 | 1H NMR (400 MHz, METHANOL-d4) δ: 7.41 (s, 1 H) 7.19 (s, 1 H) 1.99 (s, | MS (ESI): m/z = |
| | 3 H) 6.10 (t, J = 6.32 Hz, 1 H) 4.61 (t, J = 8.69 Hz, 2 H) 3.90 (d, J = 6.50 Hz, 2 | 366.1 [M + H] |
| | H) 3.43-3.52 (m, 2 H) 3.25 (t, J = 8.69 Hz, 2 H) 3.01 (s, 3 H) 2.81-2.90 (m, | |
| | 2 H) 2.33 (s, 3 H) 2.02-2.10 (m, 2 H) | |
| 288 | 1H NMR (400 MHz, METHANOL-d4) δ: 7.14 (br s, 1H) 6.97 (br s, 1H) 6.15- | MS (ESI): m/z = |
| | 6.00 (m, 2H) 5.57 (br s, 1H) 4.63 (br t, J = 8.7 Hz, 2H) 3.89 (d, J = 6.5 Hz, | 351.1 [M + H] |
| | 2H) 3.50-3.43 (m, 2H) 3.26 (br t, J = 8.6 Hz, 2H) 2.91-2.80 (m, 3H) 2.75 | |
| | (br s, 2H) 2.41-2.27 (m, 3H) 2.09-2.00 (m, 2H) | |
| 290 | 1H NMR (400 MHz, METHANOL-d4) δ: 7.34 (br d, J = 7.34 Hz, 1 H) 6.01 | MS (ESI): m/z = |
| | (s, 1 H) 5.97 (br t, J = 6.54 Hz, 1 H) 4.65 (br t, J = 8.37 Hz, 2 H) 3.92-4.14 | 428.1 [M + H] |
| | (m, 2 H) 3.68 (br s, 1 H) 3.44-3.54 (m, 5 H) 3.31 (s, 3 H) 3.24 (br t, J = 8.62 | |
| | Hz, 2 H) 2.93 (s, 3 H) 2.73 (br d, J = 8.31 Hz, 2 H) 2.29 (s, 3 H) 2.10 (br s, | |
| | 2 H) | |
| 292 | 1H NMR (400 MHz, METHANOL-d4) δ: 8.54 (br s, 2 H) 7.48 (s, 1 H) 7.21 | MS (ESI): m/z = |
| | (br s, 1 H) 6.04 (br t, J = 6.69 Hz, 1 H) 5.94 (br s, 1 H) 4.59 (t, J = 8.75 Hz, 2 | 436.2 [M + H] |
| | H) 4.03-4.12 (m, 1 H) 3.99 (br d, J = 6.63 Hz, 2 H) 3.80-3.89 (m, 1 H) | |
| | 3.70-3.78 (m, 1 H) 3.44-3.58 (m, 4 H) 3.23 (br t, J = 8.63 Hz, 2 H) 2.90 (s, | |
| | 3 H) 2.82-2.87 (m, 2 H) 2.26 (s, 3 H) 2.04-2.12 (m, 2 H) 1.87-2.00 (m, 3 | |
| | H) 1.58-1.66 (m, 1 H) | |
| 293 | 1H NMR (400 MHz, METHANOL-d4) δ: 7.17-7.05 (m, 1H) 7.02-6.90 (m, | MS (ESI): m/z = |
| | 1H) 6.27-5.97 (m, 2H) 5.83-5.56 (m, 1H) 4.67-4.58 (m, 2H) 3.98-3.85 | 409.1 [M + H] |
| | (m, 2H) 3.52-3.32 (m, 5H) 3.27 (br d, J = 3.0 Hz, 3H) 2.94-2.81 (m, 2H) | |
| | 2.46-2.23 (m, 3H) 1.99 (br s, 2H) 1.86-1.74 (m, 2H) | |
| 294 | 1H NMR (400 MHz, METHANOL-d4) δ: 8.54 (br s, 2 H) 7.48 (s, 1 H) 7.22 | MS (ESI): m/z = |
| | (br s, 1 H) 6.03-6.17 (m, 1 H) 5.93 (br s, 1 H) 4.58 (t, J = 8.69 Hz, 2 H) 4.07 | 422.1 [M + H] |
| | (quin, J = 6.28 Hz, 1 H) 3.87-3.92 (m, 2 H) 3.86 (br d, J = 1.38 Hz, 1 H) | |
| | 3.68-3.79 (m, 1 H) 3.38-3.57 (m, 4 H) 3.22 (t, J = 8.63 Hz, 2 H) 2.80-2.90 | |
| | (m, 2 H) 2.26 (s, 3 H) 2.01-2.10 (m, 2 H) 1.83 (br s, 3 H) 1.56-1.68 (m, 1 | |
| | H) | |
| 295 | 1H NMR (400 MHz, METHANOL-d4) δ: 7.18-7.05 (m, 1H) 7.00-6.87 (m, | MS (ESI): m/z = |
| | 1H) 6.27-6.13 (m, 1H) 6.09-5.98 (m, 1H) 5.79-5.60 (m, 1H) 4.68-4.57 | 423.2 [M + H] |
| | (m, 2H) 4.15-3.89 (m, 1H) 3.67 (br s, 5H) 3.11 (br s, 7H) 2.94 (d, J = 17.4 | |
| | Hz, 5H) 2.71-2.63 (m, 1H) 2.43-2.23 (m, 3H) 2.19-1.96 (m, 1H) 1.88- | |
| | 1.71 (m, 2H) | |
| 296 | 1H NMR (400 MHz, METHANOL-d4) δ: 7.42 (s, 1 H) 7.21 (br s, 1 H) 6.07 | MS (ESI): m/z = |
| | (br t, J = 6.69 Hz, 1 H) 5.95 (s, 1 H) 4.60-4.67 (m, 2 H) 3.89-4.19 (m, 2 H) | 383.2 [M + H] |
| | 3.52-3.75 (m, 2 H) 3.44 (br s, 2 H) 3.25-3.30 (m, 2 H) 2.94 (s, 3 H) 2.87 | |
| | (br s, 2 H) 2.30 (s, 3 H) 2.12 (br d, J = 11.63 Hz, 2 H) | |
| 297 | 1H NMR (400 MHz, METHANOL-d4) δ: 7.14 (br s, 1H) 6.96 (br s, 1H) 6.10 | MS (ESI): m/z = |
| | (br s, 1H) 6.04 (br t, J = 6.6 Hz, 1H) 5.87-5.56 (m, 1H) 4.63 (br t, J = 8.8 | 409.1 [M + H] |
| | Hz, 2H) 4.13-4.03 (m, 1H) 4.01-3.89 (m, 1H) 3.68 (br d, J = 11.6 Hz, 1H) | |
| | 3.54-3.50 (m, 2H) 3.37 (br s, 2H) 3.33 (s, 3H) 3.26 (br t, J = 8.6 Hz, 3H) | |
| | 2.92 (s, 3H) 2.84 (br t, J = 5.4 Hz, 2H) 2.32 (br s, 3H) 2.20-1.96 (m, 2H) | |
| 298 | 1H NMR (400 MHz, METHANOL-d4) δ: 7.46-7.40 (m, 1H) 7.20-7.14 (m, | MS (ESI): m/z = |
| | 1H) 6.10-6.01 (m, 1H) 5.97-5.91 (m, 1H) 4.64-4.59 (m, 2H) 3.97 (br s, | 424.2 [M + H] |
| | 2H) 3.74-3.61 (m, 1H) 3.51-3.40 (m, 5H) 3.28 (s, 3H) 3.27 (br s, 2H) 2.94- | |
| | 2.90 (m, 3H) 2.89-2.82 (m, 2H) 2.28 (s, 3H) 2.18-1.98 (m, 2H) 1.87- | |
| | 1.80 (m, 2H) | |
| 299 | 1H NMR (400 MHz, METHANOL-d4) δ: 7.41-7.33 (m, 1H) 7.11-7.02 (m, | MS (ESI): m/z = |
| | 1H) 6.04-5.96 (m, 1H) 5.92-5.86 (m, 1H) 4.61-4.53 (m, 2H) 4.17-3.99 | 450.2 [M + H] |
| | (m, 1H) 3.91 (br s, 3H) 3.69-3.55 (m, 1H) 3.33 (br s, 3H) 3.29-3.25 (m, | |
| | 2H) 3.23-3.16 (m, 2H) 2.96-2.86 (m, 3H) 2.80 (br s, 2H) 2.37-2.18 (m, | |
| | 3H) 2.17-1.88 (m, 2H) 1.85-1.73 (m, 1H) 1.66-1.48 (m, 2H) 1.29-1.12 | |
| | (m, 2H) | |
| 300 | 1H NMR (400 MHz, METHANOL-d4) δ: 7.29-7.37 (m, 1 H) 7.16 (br s, 1 | MS (ESI): m/z = |
| | H) 6.07-6.20 (m, 2 H) 5.93 (s, 1 H) 4.60-4.64 (m, 2 H) 3.90 (d, J = 6.78 Hz, | 369.1 [M + H] |
| | 1 H) 3.46-3.49 (m, 1 H) 3.43 (br s, 2 H) 3.25 (br d, J = 2.76 Hz, 2 H) 2.81- | |
| | 2.93 (m, 3 H) 2.61-2.67 (m, 1 H) 2.27 (s, 3 H) 1.97-2.12 (m, 2 H) | |
| 301 | 1H NMR (400 MHz, METHANOL-d4) δ: 7.43-7.33 (m, 1H) 7.25-7.14 (m, | MS (ESI): m/z = |
| | 1H) 6.06 (t, J = 6.8 Hz, 1H) 5.98-5.90 (m, 1H) 4.71-4.50 (m, 2H) 4.17- | 369.2 [M + H] |
| | 3.88 (m, 1H) 3.77-3.53 (m, 1H) 3.29-3.21 (m, 2H) 2.97 (s, 3H) 2.88-2.62 | |
| | (m, 2H) 2.49-2.18 (m, 3H) 2.16-2.01 (m, 1H) | |
| 302 | 1H NMR (400 MHz, METHANOL-d4) δ: 7.39 (s, 1H) 7.19 (s, 1H) 6.10 (t, | MS (ESI): m/z = |
| | J = 6.4 Hz, 1H) 5.95 (s, 1H) 4.64-4.59 (m, 2H) 3.90 (d, J = 6.4 Hz, 2H) 3.49- | 353.1 [M + H] |
| | 3.45 (m, 2H) 3.27-3.23 (m, 2H) 2.96 (s, 3H) 2.87-2.83 (m, 2H) 2.26 (br s, | |
| | 2H) 2.07-2.03 (m, 2H) | |
| 303 | 1H NMR (400 MHz, METHANOL-d4) δ: 7.41 (s, 1H) 7.20 (s, 1H) 6.06 (t, | MS (ESI): m/z = |
| | J = 6.8 Hz, 1H) 5.95 (s, 1H) 4.62 (br t, J = 8.8 Hz, 2H) 4.11-3.92 (m, 2H) 3.67 | 367.1 [M + H] |
| | (br s, 1H) 3.44 (br s, 1H) 3.25 (br t, J = 8.7 Hz, 2H) 2.96 (s, 3H) 2.92 (s, 3H) | |
| | 2.86 (br d, J = 4.3 Hz, 2H) 2.28-2.24 (m, 2H) 2.11 (br s, 2H) | |
| 304 | 1H NMR (400 MHz, METHANOL-d4) δ: 1.89-1.99 (m, 2 H) 2.25 (d, | MS (ESI): m/z = |
| | J = 3.13 Hz, 3 H) 2.71-2.76 (m, 5 H) 3.13-3.17 (m, 2 H) 3.36 (br t, J = 5.63 | 369.1 [M + H] |
| | Hz, 2 H) 3.79 (d, J = 6.38 Hz, 2 H) 4.53 (t, J = 8.76 Hz, 2 H) 5.61 (d, J = 6.75 | |
| | Hz, 1 H) 5.98 (t, J = 6.44 Hz, 1 H) 6.87 (d, J = 1.88 Hz, 1 H) 7.05 (s, 1 H) | |
| 305 | 1H NMR (400 MHz, METHANOL-d4) δ: 7.13 (br s, 1H) 6.95 (br s, 1H) 6.08 | MS (ESI): m/z = |
| | (t, J = 6.3 Hz, 1H) 4.63 (t, J = 8.8 Hz, 2H) 3.89 (d, J = 6.5 Hz, 2H) 3.52 (t, J = | 395.1 [M + H] |
| | 5.3 Hz, 2H) 3.49-3.45 (m, 2H) 3.36-3.32 (m, 4H) 3.28 (br s, 3H) 2.87- | |
| | 2.82 (m, 2H) 2.32 (br s, 3H) 2.04 (s, 2H) | |
| 306 | 1H NMR (400 MHz, METHANOL-d4) δ: 7.45-7.32 (m, 1H) 7.23-7.10 (m, | MS (ESI): m/z = |
| | 1H) 6.20-6.15 (m, 1H) 6.12-6.08 (m, 1H) 6.20-6.05 (m, 1H) 5.96-5.93 | 366.2 [M + H] |
| | (m, 1H) 5.98-5.92 (m, 1H) 4.67-4.55 (m, 2H) 3.91-3.88 (m, 1H) 3.50- | |
| | 3.44 (m, 1H) 3.38-3.32 (m, 1H) 3.30-3.17 (m, 3H) 2.96-2.81 (m, 2H) 2.71- | |
| | 2.58 (m, 1H) 2.42-2.18 (m, 3H) 2.08-2.01 (m, 1H) | |
| 307 | 1H NMR (400 MHz, METHANOL-d4) δ: 1.77 (s, 3 H) 2.03 (br s, 2 H) 2.28 | MS (ESI): m/z = |
| | (s, 3 H) 2.62-2.73 (m, 2 H) 2.94 (s, 3 H) 3.27 (br t, J = 8.63 Hz, 2 H) 3.43 (br | 355.2 [M + H] |
| | t, J = 5.50 Hz, 2 H) 3.86 (s, 2 H) 4.60 (t, J = 8.69 Hz, 2 H) 5.95 (s, 1 H) 7.01 (s, | |
| | 1 H) 7.41 (s, 1 H) | |
| 308 | 1H NMR (400 MHz, METHANOL-d4) δ: 7.52-7.31 (m, 1H) 7.18-7.05 (m, | MS (ESI): m/z = |
| | 1H) 6.16-6.03 (m, 1H) 6.00-5.88 (m, 1H) 4.64-4.58 (m, 2H) 3.96-3.87 | 436.2 [M + H] |
| | (m, 4H) 3.52-3.41 (m, 2H) 3.40-3.35 (m, 2H) 3.28-3.19 (m, 3H) 2.93- | |
| | 2.82 (m, 2H) 2.42-2.16 (m, 4H) 2.16-1.94 (m, 2H) 1.88-1.81 (m, 1H) 1.69- | |
| | 1.57 (m, 2H) 1.31-1.22 (m, 2H) | |
| 309 | 1H NMR (400 MHz, METHANOL-d4) δ: 2.05 (br s, 2 H) 2.38 (br s, 3 H) | MS (ESI): m/z = |
| | 2.84 (br d, J = 8.56 Hz, 2 H) 3.26 (s, 2 H) 3.42-3.52 (m, 2 H) 3.87-4.05 (m, | 419.1 [M + H] |
| | 4 H) 4.64 (br t, J = 8.80 Hz, 2 H) 5.88 (br s, 1 H) 6.00-6.12 (m, 1 H) 6.17- | |
| | 6.35 (m, 1 H) 6.94-6.99 (m, 1 H) 7.13 (s, 1 H) | |
| 310 | 1H NMR (400 MHz, METHANOL-d4) δ: 7.46-7.34 (m, 1H) 7.17-7.09 (m, | MS (ESI): m/z = |
| | 1H) 6.10 (t, J = 6.5 Hz, 1H) 5.95 (s, 1H) 4.62 (br t, J = 8.7 Hz, 2H) 3.90 | 369.1 [M + H] |
| | (d, J = 6.5 Hz, 2H) 3.51-3.45 (m, 4H) 3.41 (t, J = 6.1 Hz, 2H) 3.29 (s, 3H) | |
| | 3.25 (s, 2H) 2.89-2.80 (m, 2H) 2.28 (s, 3H) 2.11-2.00 (m, 2H) 1.86-1.77 | |
| | (m, 2H) | |
| 311 | 1H NMR (400 MHz, METHANOL-d4) δ: 2.03-2.10 (m, 2 H) 2.31-2.42 | MS (ESI): m/z = |
| | (m, 3 H) 2.79-2.85 (m, 2 H) 2.97-3.01 (m, 3 H) 3.47-3.53 (m, 2 H) 3.91- | 352.1 [M + H] |
| | 3.96 (m, 2 H) 5.07-5.10 (m, 2 H) 5.11 (br s, 2 H) 5.87-5.93 (m, 1 H) 6.03- | |
| | 6.06 (m, 1 H) 7.40-7.44 (m, 1 H) 7.50-7.55 (m, 1 H) | |
| 312 | 1H NMR (400 MHz, METHANOL-d4) δ: 7.19-7.09 (m, 1H) 7.02-6.90 (m, | MS (ESI): m/z = |
| | 1H) 6.15-5.98 (m, 2H) 5.91-5.29 (m, 1H) 4.88 (br s, 2H) 4.69-4.61 (m, | 407.1 [M + H] |
| | 2H) 4.58-4.50 (m, 2H) 4.18-4.04 (m, 1H) 4.00-3.88 (m, 1H) 3.75-3.61 | |
| | (m, 1H) 3.52-3.39 (m, 1H) 3.30-3.21 (m, 2H) 3.19-2.90 (m, 4H) 2.85 (br | |
| | t, J = 5.5 Hz, 2H) 2.36 (br s, 3H) 2.20-1.97 (m, 2H) | |
| 313 | 1H NMR (400 MHz, METHANOL-d4) δ: 7.18-7.07 (m, 1H) 7.01-6.89 (m, | MS (ESI): m/z = |
| | 1H) 6.20-6.00 (m, 2H) 5.92-5.27 (m, 1H) 5.02-4.88 (m, 2H) 4.65 (br | 393.1 [M + H] |
| | t, J = 8.7 Hz, 2H) 4.58-4.49 (m, 2H) 3.90 (br d, J = 6.1 Hz, 2H) 3.51-3.45 | |
| | (m, 2H) 3.39-3.32 (m, 1H) 3.27 (br t, J = 8.7 Hz, 2H) 2.94-2.81 (m, 2H) | |
| | 2.36 (br s, 3H) 2.11-2.00 (m, 2H) | |
| 314 | 1H NMR (400 MHz, METHANOL-d4) δ: 7.43 (br s, 1H) 7.26-7.15 (m, 1H) | MS (ESI): m/z = |
| | 6.06 (t, J = 6.7 Hz, 1H) 5.75 (s, 1H) 4.65-4.59 (m, 2H) 4.16-3.90 (m, 2H) | 392.1 [M + H] |
| | 3.69-3.43 (m, 2H) 3.25 (br t, J = 8.8 Hz, 2H) 2.96-2.94 (m, 3H) 2.92 (s, | |
| | 3H) 2.90-2.81 (m, 2H) 2.21-1.98 (m, 2H) 1.94-1.85 (m, 1H) 1.22-1.15 | |
| | (m, 2H) 1.01-0.88 (m, 2H) | |
| 315 | 1H NMR (400 MHz, METHANOL-d4) δ: 7.53-7.15 (m, 2H) 6.03 (br s, 1H) | MS (ESI): m/z = |
| | 5.95-5.88 (m, 1H) 4.73-4.54 (m, 2H) 4.16 (br d, J = 1.3 Hz, 1H) 3.62-3.35 | 366.1 [M + H] |
| | (m, 5H) 3.33-3.08 (m, 4H) 2.64 (br s, 2H) 2.29 (br s, 3H) 1.23 (br t, J = 7.2 | |
| | Hz, 3H) | |
| 316 | 1H NMR (400 MHz, METHANOL-d4) δ: 1.28 (t, J = 7.57 Hz, 3 H) 2.02- | MS (ESI): m/z = |
| | 2.18 (m, 2 H) 2.60 (q, J = 7.55 Hz, 2 H) 2.87 (br s, 2 H) 2.93 (s, 3 H) 2.97 (s, | 380.1 [M + H] |
| | 3 H) 3.26 (br t, J = 8.82 Hz, 2 H) 3.41-3.73 (m, 2 H) 3.92-4.14 (m, 2 H) | |
| | 4.61-4.66 (m, 2 H) 5.98 (s, 1 H) 6.07 (t, J = 6.82 Hz, 1 H) 7.22 (s, 1 H) 7.42 | |
| | (s, 1 H). | |
| 317 | 1H NMR (400 MHz, METHANOL-d4) δ: 7.43 (s, 1H) 7.28-7.18 (m, 1H) | MS (ESI): m/z = |
| | 6.10 (t, J = 6.5 Hz, 1H) 5.77-5.70 (m, 1H) 4.64-4.58 (m, 2H) 3.92-3.86 | 378.1 [M + H] |
| | (m, 2H) 3.49-3.44 (m, 2H) 3.24 (br t, J = 8.7 Hz, 2H) 2.98-2.93 (m, 3H) | |
| | 2.88-2.80 (m, 2H) 2.09-2.01 (m, 2H) 1.94-1.85 (m, 1H) 1.22-1.14 (m, | |
| | 2H) 1.02-0.90 (m, 2H) | |
| 318 | 1H NMR (400 MHz, METHANOL-d4) δ: 7.48-7.37 (m, 1H) 7.21-7.09 (m, | MS (ESI): m/z = |
| | 1H) 6.10-6.01 (m, 1H) 5.96-5.85 (m, 1H) 4.67-4.59 (m, 2H) 4.47-4.37 | 406.2 [M + H] |
| | (m, 1H) 4.17 (br s, 2H) 3.73-3.38 (m, 2H) 3.29-3.22 (m, 2H) 2.97 (s, 3H) | |
| | 2.89-2.78 (m, 2H) 2.74-2.51 (m, 1H) 2.35-2.26 (m, 4H) 2.19-1.95 (m, | |
| | 4H) 1.85 (br s, 2H) | |
| 319 | 1H NMR (400 MHz, METHANOL-d4) δ: 1.29 (t, J = 7.50 Hz, 3 H) 2.04- | MS (ESI): m/z = |
| | 2.10 (m, 2 H) 2.61 (q, J = 7.42 Hz, 2 H) 2.83-2.89 (m, 2 H) 2.98 (s, 3 H) | 366.1 [M + H] |
| | 3.27 (br t, J = 8.57 Hz, 2 H) 3.46-3.51 (m, 2 H) 3.92 (d, J = 6.50 Hz, 2 H) | |
| | 4.63 (t, J = 8.76 Hz, 2 H) 5.99 (s, 1 H) 6.12 (br t, J = 6.50 Hz, 1 H) 7.23 (br s, | |
| | 1 H) 7.43 (br s, 1 H). | |
| 320 | 1H NMR (400 MHz, METHANOL-d4) δ: 2.11 (br s, 2 H) 2.27 (s, 3 H) 2.86 | MS (ESI): m/z = |
| | (br d, J = 8.00 Hz, 2 H) 2.90-2.96 (m, 3 H) 3.23 (t, J = 8.69 Hz, 2 H) 3.56 | 434.1 [M + H] |
| | (br s, 2 H) 4.01 (br s, 2 H) 4.17 (q, J = 9.34 Hz, 2 H) 4.56-4.64 (m, 2 H) | |
| | 5.99 (s, 1 H) 6.05 (br s, 1 H) 7.22 (br s, 1 H) 7.46 (s, 1 H) 8.46 (s, 2 H). | |
| 321 | 1H NMR (400 MHz, METHANOL-d4) δ: 7.53-7.29 (m, 1H) 7.26-7.09 (m, | MS (ESI): m/z = |
| | 1H) 6.06 (br t, J = 6.3 Hz, 1H) 5.94 (s, 1H) 4.65-4.59 (m, 2H) 4.13-3.89 | 380.1 [M + H] |
| | (m, 2H) 3.73-3.59 (m, 1H) 3.50-3.39 (m, 3H) 3.25 (br t, J = 8.6 Hz, 2H) | |
| | 2.93 (s, 3H) 2.88-2.83 (m, 2H) 2.28 (s, 3H) 2.10 (br s, 2H) 1.21 (t, J = 7.3 | |
| | Hz, 3H) | |
| 322 | 1H NMR (400 MHz, METHANOL-d4) δ: 2.04-2.10 (m, 2 H) 2.35 (s, 3 H) | MS (ESI): m/z = |
| | 2.83-2.87 (m, 2 H) 3.25 (t, J = 8.69 Hz, 2 H) 3.46-3.50 (m, 2 H) 3.89 (d, J = | 420.1 [M + H] |
| | 6.50 Hz, 2 H) 4.19 (q, J = 9.46 Hz, 2 H) 4.63 (t, J = 8.76 Hz, 2 H) 6.04- | |
| | 6.13 (m, 2 H) 7.12 (s, 1 H) 7.34 (br s, 1 H). | |
| 323 | 1H NMR (400 MHz, METHANOL-d4) δ: 7.45 (s, 1H) 7.26 (s, 1H) 6.06 (br t, | MS (ESI): m/z = |
| | J = 6.7 Hz, 1H) 5.98 (s, 1H) 4.65-4.59 (m, 2H) 4.13-3.93 (m, 2H) 3.67 (br s, | 394.1 [M + H] |
| | 1H) 3.44 (br t, J = 9.9 Hz, 1H) 3.25 (br t, J = 8.7 Hz, 2H) 2.97 (s, 3H) 2.92 (s, | |
| | 3H) 2.88-2.82 (m, 3H) 2.08 (br d, J = 18.8 Hz, 2H) 1.30 (d, J = 6.9 Hz, 6H) | |
| 324 | 1H NMR (400 MHz, METHANOL-d4) δ: 2.02-2.16 (m, 2 H) 2.29 (s, 3 H) | MS (ESI): m/z = |
| | 2.85 (br d, J = 4.50 Hz, 2 H) 2.92 (s, 3 H) 3.25 (br t, J = 8.76 Hz, 2 H) 3.35- | 410.1 [M + H] |
| | 3.36 (m, 3 H) 3.45 (br d, J = 3.50 Hz, 1 H) 3.54-3.50 (m, 2 H) 3.61-3.58 | |
| | (m, 2 H) 3.66-3.68 (m, 1 H) 3.93-4.15 (m, 2 H) 4.59-4.65 (m, 2 H) 5.99 | |
| | (s, 1 H) 6.05 (t, J = 6.82 Hz, 1 H) 7.17 (d, J = 1.63 Hz, 1 H) 7.38 (s, 1 H) | |
| | 1H NMR (400 MHz, METHANOL-d4) δ: 2.03-2.15 (m, 2 H) 2.29 (s, 3 H) | |
| | 2.85 (br d, J = 4.50 Hz, 2 H) 2.92 (s, 3 H) 3.25 (br t, J = 8.76 Hz, 2 H) 3.33- | |
| | 3.36 (m, 3 H) 3.44 (br d, J = 3.50 Hz, 1 H) 3.51-3.55 (m, 2 H) 3.57-3.61 | |
| | (m, 2 H) 3.65-3.70 (m, 1 H) 3.93-4.12 (m, 2 H) 4.59-4.65 (m, 2 H) 5.99 | |
| | (s, 1 H) 6.05 (t, J = 6.82 Hz, 1 H) 7.17 (d, J = 1.63 Hz, 1 H) 7.39 (s, 1 H) | |
| 325 | 1H NMR (400 MHz, METHANOL-d4) δ: 7.44-7.34 (m, 1H) 7.20-7.09 (m, | MS (ESI): m/z = |
| | 1H) 6.16-6.05 (m, 1H) 5.95-5.86 (m, 1H) 4.66-4.60 (m, 2H) 4.39 (br s, | 392.1 [M + H] |
| | 1H) 3.97-3.83 (m, 2H) 3.52-3.43 (m, 2H) 3.35-3.29 (m, 6H) 3.28-3.23 | |
| | (m, 2H) 2.91 (br s, 2H) 2.38-2.30 (m, 2H) 2.29-2.22 (m, 3H) 2.10-1.98 | |
| | (m, 4H) 1.85 (br s, 2H) | |
| 326 | 1H NMR (400 MHz, METHANOL-d4) δ: 7.45 (s, 1H) 7.25 (d, J = 1.8 Hz, | MS (ESI): m/z = |
| | 1H) 6.10 (t, J = 6.4 Hz, 1H) 5.98 (s, 1H) 4.63-4.58 (m, 2H) 3.90 (d, J = 6.5 | 380.1 [M + H] |
| | Hz, 2H) 3.49-3.44 (m, 2H) 3.37-3.31 (m, 1H) 3.24 (br t, J = 8.7 Hz, 2H) | |
| | 2.97 (s, 3H) 2.87-2.83 (m, 2H) 2.08-2.02 (m, 2H) 1.29 (d, J = 6.9 Hz, 6H) | |
| 327 | 1H NMR (400 MHz, METHANOL-d4) δ: 2.06 (m, 2 H) 2.47 (s, 3 H) 2.80 (s, | MS (ESI): m/z = |
| | 3 H) 2.85 (m, 2 H) 3.25 (m, 2 H) 3.47 (m, 2 H) 3.90 (d, J = 6.63 Hz, 2 H) | 352.1 [M + H] |
| | 4.61 (br t, J = 8.57 Hz, 2 H) 6.11 (t, J = 6.57 Hz, 1 H) 7.12 (s, 1 H) 7.30 (br s, | |
| | 1 H) 7.35 (s, 1 H) | |
| 328 | 1H NMR (400 MHz, METHANOL-d4) δ: 7.18 (br d, J = 6.5 Hz, 1H) 6.27- | MS (ESI): m/z = |
| | 5.98 (m, 2H) 5.60-5.39 (m, 1H) 4.66 (br t, J = 8.6 Hz, 2H) 3.92 (d, J = 6.4 | 369.1 [M + H] |
| | Hz, 2H) 3.54-3.43 (m, 2H) 3.30-3.19 (m, 2H) 2.76-2.72 (m, 3H) 2.47- | |
| | 2.24 (m, 3H) 2.16-1.99 (m, 2H) | |
| 329 | 1H NMR (400 MHz, METHANOL-d4) δ: 7.14 (br s, 1H) 7.04-6.88 (m, 1H) | MS (ESI): m/z = |
| | 6.04-5.87 (m, 1H) 4.32-4.17 (m, 4H) 3.66-3.40 (m, 2H) 3.27-3.14 (m, | 396.1 [M + H] |
| | 2H) 3.13-3.03 (m, 2H) 3.01-2.92 (m, 7H) 2.38-2.32 (m, 2H) 2.31-2.18 | |
| | (m, 3H) 1.68-1.47 (m, 2H) | |
| 330 | 1H NMR (400 MHz, METHANOL-d4) δ: 7.52 (br d, J = 7.25 Hz, 1 H) 7.32 | MS (ESI): m/z = |
| | (br s, 1 H) 7.14 (br s, 1 H) 6.10-6.17 (m, 2 H) 4.65 (t, J = 8.76 Hz, 2 H) 3.92 | 338.1 [M + H] |
| | (d, J = 6.63 Hz, 2 H) 3.46-3.52 (m, 2 H) 3.29 (br d, J = 8.76 Hz, 2 H) 3.01 (s, 3 | |
| | H) 2.84-2.89 (m, 2 H) 2.03-2.11 (m, 2 H) | |
| 331 | 1H NMR (400 MHz, METHANOL-d4) δ: 7.78 (br s, 1 H) 7.42 (br s, 1 H) | MS (ESI): m/z = |
| | 7.10 (br s, 1 H) 6.03-6.14 (m, 2 H) 3.96-4.19 (m, 2 H) 3.43-3.77 (m, 2 H) | 352.1 [M + H] |
| | 3.08 (s, 3 H) 3.03 (s, 3 H) 2.97 (s, 3 H) 2.93 (br s, 2 H) 2.35 (s, 3 H) 2.01- | |
| | 2.26 (m, 2 H) | |
| 332 | 1H NMR (400 MHz, METHANOL-d4) δ: 7.21 (d, J = 1.5 Hz, 1H) 6.91 (d, J = | MS (ESI): m/z = |
| | 1.9 Hz, 1H) 5.98 (s, 1H) 5.93 (t, J = 6.3 Hz, 1H) 4.60-4.55 (m, 2H) 3.91 | 352.1 [M + H] |
| | (d, J = 6.4 Hz, 2H) 3.51-3.47 (m, 2H) 3.21 (t, J = 8.6 Hz, 2H) 3.00 (s, 3H) | |
| | 2.83-2.80 (m, 2H) 2.29 (s, 3H) 2.08-2.03 (m, 2H) | |
| 333 | 1H NMR (400 MHz, METHANOL-d4) δ: 7.23 (s, 1H) 6.92 (d, J = 1.1 Hz, | MS (ESI): m/z = |
| | 1H) 5.98 (d, J = 0.6 Hz, 1H) 5.89 (br t, J = 6.4 Hz, 1H) 4.58 (br t, J = 8.6 Hz, | 366.1 [M + H] |
| | 2H) 4.12-3.95 (m, 2H) 3.70 (br s, 1H) 3.45 (br s, 1H) 3.25-3.20 (m, 2H) | |
| | 3.00 (s, 3H) 2.95 (s, 3H) 2.83 (br s, 2H) 2.29 (s, 3H) 2.19-2.03 (m, 2H) | |
| 334 | 1H NMR (400 MHz, METHANOL-d4) δ: 7.25-7.13 (m, 1H) 7.00-6.89 (m, | MS (ESI): m/z = |
| | 1H) 6.04-5.88 (m, 1H) 4.30-4.21 (m, 4H) 3.48 (br s, 4H) 3.28-3.22 (m, | 382.1 [M + H] |
| | 2H) 3.03-2.98 (m, 4H) 2.39-2.33 (m, 2H) 2.33-2.19 (m, 3H) 1.60-1.47 | |
| | (m, 2H) | |
| 335 | 1H NMR (400 MHz, METHANOL-d4) δ: 2.00-2.21 (m, 2 H) 2.87-2.92 | MS (ESI): m/z = |
| | (m, 2 H) 2.93 (s, 3 H) 3.00 (s, 3 H) 3.37-3.74 (m, 2 H) 3.91-4.22 (m, 2 H) | 354.1 [M + H] |
| | 6.05 (s, 2 H) 6.13-6.22 (m, 2 H) 6.94 (d, J = 1.63 Hz, 1 H) 7.12 (br s, 1 H) | |
| | 7.55 (d, J = 7.25 Hz, 1 H). | |
| 336 | 1H NMR (400 MHz, METHANOL-d4) δ: 2.01-2.09 (m, 2 H) 2.85-2.91 | MS (ESI): m/z = |
| | (m, 2 H) 3.00 (s, 3 H) 3.44-3.50 (m, 2 H) 3.92 (d, J = 6.50 Hz, 2 H) 6.01- | 340.1 [M + H] |
| | 6.05 (m, 2 H) 6.15 (d, J = 7.25 Hz, 1 H) 6.22 (t, J = 6.44 Hz, 1 H) 6.92 (d, J = | |
| | 1.88 Hz, 1 H) 7.11 (br s, 1 H) 7.55 (br d, J = 7.25 Hz, 1 H) | |
| 337 | 1H NMR (400 MHz, METHANOL-d4) δ: 7.30-7.24 (m, 1H) 6.96-6.84 (m, | MS (ESI): m/z = |
| | 1H) 5.99-5.89 (m, 1H) 4.86 (s, 14H) 4.34-4.19 (m, 4H) 3.60-3.52 (m, 1H) | 370.1 [M + H] |
| | 3.35-3.26 (m, 3H) 3.02-2.98 (m, 2H) 3.09-2.92 (m, 4H) 2.39-2.23 (m, | |
| | 3H) 2.05-1.88 (m, 4H) 1.88-1.68 (m, 4H) | |
| 338 | 1H NMR (400 MHz, METHANOL-d4) δ: 6.93 (br d, J = 11.0 Hz, 2H) 5.94 | MS (ESI): m/z = |
| | (d, J = 0.8 Hz, 1H) 4.27-4.24 (m, 2H) 3.61-3.56 (m, 2H) 3.52 (br d, J = 9.9 | 381.1 [M + H] |
| | Hz, 2H) 3.21-3.17 (m, 2H) 3.11 (br s, 2H) 2.99 (s, 3H) 2.96-2.92 (m, 2H) | |
| | 2.81 (br t, J = 6.4 Hz, 2H) 2.27 (s, 3H) 2.04-1.99 (m, 2H) | |
| 339 | 1H NMR (400 MHz, METHANOL-d4) δ: 7.01 (br s, 1H) 6.92 (br s, 1H) 5.94 | MS (ESI): m/z = |
| | (s, 1H) 4.26-4.23 (m, 2H) 3.49-3.42 (m, 4H) 3.39 (br dd, J = 5.3, 8.0 Hz, | 369.1 [M + H] |
| | 4H) 2.98 (s, 3H) 2.81 (br t, J = 6.4 Hz, 2H) 2.27 (s, 3H) 2.21-2.16 (m, 2H) | |
| | 2.05-2.00 (m, 2H) | |
| 340 | 1H NMR (400 MHz, METHANOL-d4) δ: 7.18 (s, 2H) 5.93 (s, 1H) 4.23- | MS (ESI): m/z = |
| | 4.19 (m, 2H) 3.42-3.34 (m, 2H) 3.26-3.20 (m, 2H) 3.05-2.92 (m, 6H) 2.81 | 380.1 [M + H] |
| | (br t, J = 6.4 Hz, 2H) 2.38-2.26 (m, 5H) 2.03-1.96 (m, 2H) 1.50 (br d, J = | |
| | 8.4 Hz, 2H) | |
| 341 | 1H NMR (400 MHz, METHANOL-d4) δ: 7.19 (s, 1H) 7.15 (br s, 1H) 5.94 | MS (ESI): m/z = |
| | (s, 1H) 4.25-4.19 (m, 2H) 3.48-3.34 (m, 2H) 3.28-3.17 (m, 3H) 2.99- | 368.1 [M + H] |
| | 2.93 (m, 1H) 2.81 (t, J = 6.4 Hz, 2H) 2.27 (s, 3H) 2.12-1.96 (m, 6H) 1.94- | |
| | 1.73 (m, 2H) | |
| 342 | 1H NMR (400 MHz, METHANOL-d4) δ: 1.64 (td, J = 12.29, 8.69 Hz, 2 H) | MS (ESI): m/z = |
| | 2.28 (s, 3 H) 2.39 (m, 2 H) 2.97 (s, 3 H) 3.02 (m, 2 H) 3.20 (m, 1 H) 3.26 (m, | 368.1 [M + H] |
| | 2 H) 3.36 (m, 2 H) 5.95 (s, 1 H) 5.98 (s, 2 H) 6.88 (d, J = 1.75 Hz, 1 H) 7.15 | |
| | (s, 1 H) | |
| 343 | 1H NMR (400 MHz, METHANOL-d4) δ: 7.17 (s, 1H) 6.83 (d, J = 1.9 Hz, | MS (ESI): m/z = |
| | 1H) 5.99 (s, 2H) 5.97-5.96 (m, 1H) 3.49-3.41 (m, 2H) 3.37 (br s, 1H) 3.27- | 356.1 [M + H] |
| | 3.21 (m, 2H) 2.98 (s, 3H) 2.28 (s, 3H) 2.20-2.16 (m, 2H) 2.11 (br d, J = | |
| | 11.0 Hz, 2H) 1.95-1.89 (m, 2H) | |
| 344 | 1H NMR (400 MHz, METHANOL-d4) δ: 7.28 (br d, J = 16.4 Hz, 2H) 6.28 | MS (ESI): m/z = |
| | (s, 1H) 5.94 (s, 1H) 4.29-4.22 (m, 2H) 3.81-3.73 (m, 1H) 3.54-3.39 (m, | 378.1 [M + H] |
| | 2H) 3.36-3.32 (m, 1H) 3.20-3.06 (m, 3H) 2.98 (s, 3H) 2.84 (t, J = 6.5 Hz, | |
| | 2H) 2.76 (br d, J = 14.5 Hz, 1H) 2.27 (s, 3H) 2.01 (br dd, J = 3.9, 6.1 Hz, 2H) | |
| 345 | 1H NMR (400 MHz, METHANOL-d4) δ: 7.30 (s, 1H) 7.16 (d, J = 2.1 Hz, | MS (ESI): m/z = |
| | 1H) 5.95 (s, 1H) 5.82 (br s, 1H) 4.24-4.16 (m, 2H) 3.81 (br d, J = 2.4 Hz, | 352.1 [M + H] |
| | 2H) 3.40 (t, J = 6.1 Hz, 2H) 2.97 (s, 3H) 2.84 (t, J = 6.4 Hz, 2H) 2.78 (br d, | |
| | J = 1.7 Hz, 2H) 2.28 (s, 3H) 2.05-1.95 (m, 2H) | |
| 346 | 1H NMR (400 MHz, METHANOL-d4) δ: 7.24 (br s, 1H) 7.14 (br s, 1H) 5.93 | MS (ESI): m/z = |
| | (s, 1H) 5.67 (br s, 1H) 4.20-4.14 (m, 2H) 3.44 (br s, 1H) 2.98-2.93 (m, 1H) | 366.1 [M + H] |
| | 2.81 (t, J = 6.5 Hz, 2H) 2.67-2.53 (m, 3H) 2.36 (s, 1H) 2.27 (s, 3H) 2.16- | |
| | 2.07 (m, 1H) 2.03-1.94 (m, 2H) 1.81 (br dd, J = 5.8, 11.5 Hz, 1H) | |
| 347 | 1H NMR (400 MHz, METHANOL-d4) δ: 2.29 (s, 3 H) 2.79 (dd, J = 16.38, | MS (ESI): m/z = |
| | 1.63 Hz, 1 H) 2.98 (s, 3 H) 3.11 (m, 1 H) 3.18 (m, 1 H) 3.25 (m, 1 H) 3.35 (br | 366.1 [M + H] |
| | d, J = 2.75 Hz, 1 H) 3.43 (m, 1 H) 3.51 (dd, J = 11.76, 8.63 Hz, 1 H) 3.82 (m, | |
| | 1 H) 5.97 (s, 1 H) 6.06 (d, J = 8.00 Hz, 2 H) 6.32 (br d, J = 1.38 Hz, 1 H) 6.99 | |
| | (d, J = 1.63 Hz, 1 H) 7.19 (s, 1 H) | |
| 348 | 1H NMR (400 MHz, METHANOL-d4) δ: 7.24-7.10 (m, 1H) 6.93-6.74 (m, | MS (ESI): m/z = |
| | 1H) 6.15-5.90 (m, 1H) 4.32-4.23 (m, 4H) 3.45-3.35 (m, 2H) 3.29-3.19 | 370.1 [M + H] |
| | (m, 3H) 3.01-2.92 (m, 3H) 2.38-2.27 (m, 3H) 2.15-2.03 (m, 4H) 1.98- | |
| | 1.79 (m, 2H) | |

### EXAMPLE 89. In vitro efficacy assay of exemplary compounds: EHMT1 and EHMT2 enzyme assay protocol

### Materials and Equipment

Recombinant purified human EHMT1 982-1266 (457 μM) and EHMT2 913-1193 (452 μM) produced by Biortus was used for all experiments. Biotinylated histone peptides were synthesized by AnaSpec and HPLC-purified to >95% purity. Streptavidin-coated PVT SPA Beads were purchased from PerkinElmer. 384 Well Optiplate and TopSeal-A were purchased from PerkinElmer. 384 well Echo low dead volume (LDV) microplates were purchased from Labcyte. 3H-labeled S-adenosylmethionine (3H-SAM) was obtained from PerkinElmer with a specific activity of 16 Ci/mmol. Unlabeled SAM and S-adenosyihomocysteine (SAH) were obtained from Sigma-Aldrich. 384 well Optiplate were read on a Microbeta plate reader (PerkinElmer). Compound serial dilutions were performed in 384 well LDV plates on a Echo555 (Labcyte) and transferred into assay plates (Optiplate, PerkinElmer) using Echo.

### General Procedure for EHMT1 and EHMT2 Enzyme Assay on Histone Peptide Substrate

11-point curves of test compounds were made on an Echo555 using serial 3-fold dilutions in DMSO, final top concentration of compound was 100 or 1 μM and the DMSO was 1%. A 0.5 μl aliquot of the inhibitor dilution series was spotted in a 384-well Optiplate (Perkinelmer) using Echo (Labcyte). The 100% inhibition control consisted of 100 μM final concentration of the product inhibitor S-adenosythomoeysteine (SAH, Sigma-Aldrich). Compounds were incubated for 30 minutes with 40 μl per well of 0.25 nM EHMT1 (recombinant purified human EHMT1 982-1266, Biortus) or 0.25 nM EHMT2 (recombinant purified human EHMT2 913-1193, Biortus) in 1× assay buffer (20 mM Bicine [pH 7.5], 0.002% Tween 20, 0.005% Bovine Skin Gelatin and 1 mM TCEP). 10 μl per well of substrate mix comprising assay buffer, 3H-SAM (3H-labeled S-adenosylmethionine, PerkinElmer, specific activity of 16 Ci/mmol), unlabeled SAM (Sigma-Aldrich), and peptide representing Histone 3 residues 1-21 containing C-terminal biotin (appended to a C-terminal amide-capped lysine, synthesized by AnaSpec and HPLC-purified to greater than 95% purity) were added to initiate the reaction (both substrates were present in the final reaction mixture close to their respective KM values, an assay condition referred to as “balanced conditions”). Reactions were incubated for 90 minutes at room temperature and quenched with 10 μl per well of 200 μM unlabeled SAM. 10 μl per well of 10 mg/ml SPA beads was added to the quenched reactions and incubated for 90 minutes at room temperature. The assay plates were subsequently read on Microbeta plate reader (PerkinElmer). The IC₅₀ determined for each compound using the assay is summarized in Table 3 below. The compound numbers correspond to those shown in Table 1. In the table, “A” indicates an IC₅₀ of less than 100 nM, “B” indicates an IC₅₀ range from 100 nM to 500 nM; “C” indicates an IC₅₀ range from 500 nM to 2 μM; and “D” indicates an IC₅₀ greater than 2 μM.

TABLE 3

| Efficacy of exemplary compounds of the invention | | |
|---|---|---|
| | EHMT1 IC₅₀ | EHMT2 IC₅₀ |
| Compound No. | (nM) | (nM) |
| 102 | B | C |
| 103 | B | C |
| 104 | D | D |
| 105 | A | A |
| 106 | C | C |
| 107 | B | C |
| 108 | D | D |
| 109 | D | D |
| 110 | A | A |
| 111 | A | A |
| 112 | B | D |
| 113 | B | C |
| 114 | D | D |
| 115 | D | D |
| 116 | C | D |
| 117 | C | D |
| 118 | B | C |
| 119 | C | D |
| 122 | B | B |
| 124 | B | D |
| 125 | C | C |
| 126 | C | C |
| 127 | B | C |
| 128 | C | D |
| 129 | A | A |
| 130 | A | A |
| 131 | A | B |
| 133 | B | C |
| 134 | B | B |
| 136 | B | D |
| 137 | A | A |
| 138 | B | C |
| 139 | C | D |
| 141 | A | B |
| 142 | A | A |
| 143 | B | D |
| 144 | B | B |
| 145 | C | D |
| 146 | B | C |
| 147 | C | D |
| 148 | C | D |
| 149 | D | D |
| 150 | A | B |
| 151 | A | A |
| 152 | D | D |
| 153 | D | D |
| 154 | A | C |
| 155 | B | C |
| 156 | A | B |
| 157 | B | B |
| 158 | D | D |
| 159 | A | B |
| 159-a | C | D |
| 160 | A | A |
| 161 | A | A |
| 161-a | C | D |
| 162 | C | C |
| 163 | A | A |
| 163-a | C | D |
| 164 | A | A |
| 165 | A | A |
| 166 | A | A |
| 167 | A | A |
| 168 | B | B |
| 169 | A | A |
| 170 | A | A |
| 171 | A | B |
| 172 | A | A |
| 173 | A | B |
| 174 | A | A |
| 175 | A | A |
| 176 | A | A |
| 177 (Reference | A | A |
| Compound B) | | |
| 178 | A | B |
| 179 | A | A |
| 180 | A | A |
| 181 | A | B |
| 182 | A | B |
| 183 | A | A |
| 184 | B | C |
| 185 | A | B |
| 186 | B | C |
| 187 | A | B |
| 188 | A | A |
| 189 | A | A |
| 190 | A | A |
| 191 | A | A |
| 192 | B | C |
| 193 | A | A |
| 194 | A | B |
| 195 | A | B |
| 196 | B | C |
| 197 | B | C |
| 198 | A | B |
| 199 | B | C |
| 200 | B | C |
| 201 | A | A |
| 202 | A | A |
| 203 | A | A |
| 204 | A | A |
| 205 | B | C |
| 206 | A | B |
| 207 | A | A |
| 208 | B | B |
| 209 | B | B |
| 210 | A | B |
| 211 | A | A |
| 212 | A | A |
| 214 | A | A |
| 215 | B | C |
| 216 | A | A |
| 217 | A | A |
| 218 | B | C |
| 219 | B | B |
| 220 | B | C |
| 221 | B | C |
| 222 | B | C |
| 223 | A | A |
| 224 | A | A |
| 225 | A | A |
| 226 | A | B |
| 227 | A | A |
| 228 | A | B |
| 229 | A | B |
| 230 | A | B |
| 231 | A | A |
| 232 | B | B |
| 233 | B | C |
| 234 | A | A |
| 235 | A | A |
| 236 | A | B |
| 237 | B | C |
| 238 | A | A |
| 239 | A | B |
| 240 | B | B |
| 241 | B | C |
| 242 | A | A |
| 243 | A | A |
| 244 | A | B |
| 245 | B | C |
| 246 | A | A |
| 247 | B | B |
| 248 | A | A |
| 249 | A | A |
| 250 | A | A |
| 251 | A | A |
| 252 | A | A |
| 253 | A | A |
| 254 | A | A |
| 255 | A | A |
| 256 | A | A |
| 257 | A | A |
| 258 | B | B |
| 259 | A | B |
| 260 | A | A |
| 261 | B | C |
| 262 | A | A |
| 263 | A | A |
| 264 | A | A |
| 265 | A | A |
| 266 | A | B |
| 267 | B | C |
| 268 | A | B |
| 269 | A | A |
| 270 | A | A |
| 271 | B | C |
| 272 | B | C |
| 273 | A | B |
| 274 | A | A |
| 275 | B | C |
| 276 | B | B |
| 277 | B | B |
| 278 | A | B |
| 279 | B | C |
| 280 | A | A |
| 281 | A | A |
| 282 | A | C |
| 283 | A | A |
| 284 | B | C |
| 285 | B | B |
| 286 | A | B |
| 287 | A | A |
| 288 | A | A |
| 289 | A | A |
| 290 | A | B |
| 291 | A | A |
| 292 | B | B |
| 293 | B | B |
| 294 | A | B |
| 295 | B | C |
| 296 | A | B |
| 297 | B | C |
| 298 | A | B |
| 299 | A | B |
| 300 | A | A |
| 301 | A | A |
| 302 | A | A |
| 303 | A | A |
| 304 | A | A |
| 305 | B | B |
| 306 | A | A |
| 307 | B | C |
| 308 | A | A |
| 309 | B | C |
| 310 | A | A |
| 311 | B | C |
| 312 | B | C |
| 313 | B | B |
| 314 | B | C |
| 315 | B | B |
| 316 | A | B |
| 317 | A | B |
| 318 | B | C |
| 319 | A | A |
| 320 | B | C |
| 321 | A | B |
| 322 | A | B |
| 323 | B | B |
| 324 | B | B |
| 325 | A | B |
| 326 | A | A |
| 327 | A | A |
| 328 | A | A |
| 329 | B | C |
| 330 | A | A |
| 331 | A | B |
| 332 | B | B |
| 333 | B | C |
| 334 | B | C |
| 335 | B | B |
| 336 | A | A |
| 337 | B | D |
| 338 | B | C |
| 339 | B | C |
| 340 | A | B |
| 341 | A | B |
| 342 | B | C |
| 343 | B | C |
| 344 | B | C |
| 345 | B | C |
| 346 | B | D |
| 347 | B | C |
| 348 | B | B |
| 349 (Reference | A | B |
| Compound A) | | |
| 351 | A | B |
| 352 | A | A |
| 353 | A | A |
| 354 | A | B |
| 354-a | D | D |
| 355 | A | A |
| 356 | A | A |
| 356-a | C | D |
| 357 | A | A |
| 358 | B | B |
| 359 | A | B |
| 360 | B | C |
| 363 | A | B |
| 365 | B | C |
| 366 | B | C |
| 366-a | C | D |
| 368 | B | B |
| 366-a | C | D |
| 369 | A | A |
| 370 | A | A |
| 371 | A | A |
| 371-a | D | D |
| 373 | A | A |
| 374 | A | A |
| 374-a | C | C |
| 375 | B | C |
| 376 | A | B |
| 379 | A | A |
| 380 | B | C |
| 381 | A | A |
| 382 | A | A |
| 383 | A | A |
| 383-a | C | C |
| 384 | A | A |
| 385 | A | B |
| 385-a | D | D |
| 398 | A | B |
| 401 | A | B |
| 402 | A | A |
| 403 | B | D |
| 406 | A | C |
| 407 | A | B |
| 408 | A | A |
| 409 | A | B |
| 414 | A | A |
| 415 | A | B |
| 418 | A | B |
| 419 | C | D |
| 420 | A | A |
| 422 | A | A |
| 428 | A | A |
| 434 | A | A |
| 435 | A | B |
| 436 | C | D |
| 437 | C | C |

### EXAMPLE 90. In vitro efficacy assay of exemplary compounds: H3K9me2 in-cell western assay protocol

### Materials and Equipment

HCT116 cell line was purchased from ATCC (Manassas, VA, USA). RPMI medium 1640, Trypsin, 0.25% (1×) were purchased from Gibco (Co Dublin, Ireland). Penicillin-Streptomycin and heat inactivated fetal bovine serum were purchased from Hyclone (Logan, UT, USA). DPBS, Corning^{R} were purchased from VWR (Radnor, PA, USA). Triton′ X-100 was purchased from Sigma (St. Louis, MO, USA). Intercept blocking buffer, DRAQ5, Licor Odyssey Infrared Scanner were purchased from Licor Biosciences (Lincoln, NB, USA). Plate washer was purchased from BioTek (Winooski, VT, USA). H3K9me2 rabbit mAb (Cat #4658 S) was purchased from CST (Danvers, MA, USA). 16% Paraformaldehyde was purchased from Electron Microscopy Sciences (Hatfield, PA, USA). HCT116 cells were maintained in complete growth medium (RPMI supplemented with 10% heat inactivated fetal bovine serum, 1% Penicillin-Streptomycin) and cultured at 37° C. under 5% CO₂. N²-(4-methoxy-3-(3-(pyrrolidin-1-yl)propoxy)phenyl)-N⁴,6-dimethyl]pyrimidine-2,4-diamine (synthesis disclosed in International Application WO2017181177A1) was used as a control compound.

General Procedure for HCT116 H₃K₉Me2 In-Cell Western Assay

HCT116 cells (ATCC) were trypsinized, counted and seeded in assay medium (RPMI supplemented with 10% heat inactivated fetal bovine serum, 1% Penicillin-Streptomycin) at a concentration of 2,000 cells per well to a 384-well cell culture plate with compounds in 50 μl per well. Plates were incubated with at 37° C. under 5% CO₂ for 72 hours. After the incubation, 50 μl per well of 8% paraformaldehyde in PBS was added to the plates and incubated at room temperature for 20 minutes. Plates were transferred to a BioTek (EL406) plate washer and washed 5 times with 110 μl per well of wash buffer (1×PBS containing 0.1% Triton X-100). The plates were next blocked with 50 μl of Intercept blocking buffer for 1 hour at room temperature. Blocking buffer was removed and 30 μl per well of monoclonal primary antibody of H3K9me2 (CST) diluted 1:200 in Intercept blocking buffer were added and plates were incubated overnight at 4° C. The plates were then washed 5 times with 110 μl per well of wash buffer (1×PBS containing 0.1% Triton X-100). Next 30 μl per well of secondary was added (1:400 800 CW goat anti-rabbit IgG in blocking buffer) with 1:1000 DRAQ5 in blocking buffer and incubated for 2 hours at room temperature. The plates were washed 5 times with 110 μl per well of wash buffer (1×PBS containing 0.1% Triton X-100) then with 100 μl per well of water once. Plates were allowed to dry at room temperature then imaged on a Licor Odyssey Infrared Scanner which measured integrated intensity at 700 nm and 800 nm wavelength. Both 700 and 800 channels were scanned.

The average of the ratio values (800 nm/700 nm) for each well was calculated and used to determine the percentage for each test well in the plate. Compounds were serially diluted two-fold in 0.1% DMSO for a total of 10 test concentrations beginning at 10 μM. IC₅₀ curves were generated using duplicate wells per concentration of compound. The IC₅₀ is the concentration of compound at which measured methylation is inhibited by 50% as interpolated from the dose response curves. IC₅₀ values were calculated using a non-linear regression of Prism. The IC₅₀ determined for each compound using the assay is summarized in Table 4 below. The compound numbers correspond to those shown in Table 1. In the table, “A” indicates an IC₅₀ of less than 100 nM, “B” indicates an IC₅₀ range from 100 nM to 500 nM; “C” indicates an IC₅₀ range from 500 nM to 2 μM; and “D” indicates an IC₅₀ greater than 2 μM.

TABLE 4
Efficacy of exemplary compounds of the invention

| | H3K9me2 ICW |
|---|---|
| Compound No. | IC₅₀ (μM) |
| 102 | C |
| 103 | B |
| 104 | D |
| 105 | C |
| 106 | B |
| 107 | B |
| 108 | D |
| 109 | D |
| 110 | A |
| 111 | A |
| 112 | C |
| 113 | B |
| 114 | D |
| 115 | C |
| 116 | D |
| 117 | D |
| 118 | B |
| 119 | C |
| 122 | B |
| 124 | D |
| 125 | C |
| 126 | C |
| 127 | C |
| 128 | C |
| 129 | A |
| 130 | A |
| 131 | A |
| 133 | C |
| 134 | B |
| 136 | C |
| 137 | A |
| 138 | B |
| 139 | C |
| 141 | A |
| 142 | B |
| 143 | D |
| 144 | B |
| 145 | B |
| 146 | B |
| 147 | C |
| 148 | C |
| 149 | D |
| 150 | B |
| 151 | B |
| 152 | D |
| 153 | D |
| 154 | B |
| 155 | B |
| 156 | B |
| 157 | B |
| 158 | D |
| 159 | A |
| 159-a | C |
| 160 | A |
| 161 | A |
| 161-a | C |
| 162 | C |
| 163 | A |
| 163-a | C |
| 164 | A |
| 165 | A |
| 166 | A |
| 167 | A |
| 168 | B |
| 169 | A |
| 170 | A |
| 171 | A |
| 172 | A |
| 173 | A |
| 174 | A |
| 175 | A |
| 176 | A |
| 177 (Reference | A |
| compound B) | |
| 178 | A |
| 179 | A |
| 180 | A |
| 181 | B |
| 182 | A |
| 183 | A |
| 184 | B186 |
| 185 | A |
| 186 | B |
| 187 | A |
| 188 | A |
| 189 | A |
| 190 | A |
| 191 | A |
| 192 | B |
| 193 | A |
| 194 | A |
| 195 | A |
| 196 | B |
| 197 | B |
| 198 | B |
| 199 | B |
| 200 | B |
| 201 | A |
| 202 | A |
| 203 | A |
| 204 | A |
| 205 | B |
| 206 | A |
| 207 | A |
| 208 | B |
| 209 | A |
| 210 | A |
| 211 | A |
| 212 | A |
| 213 | B |
| 214 | A |
| 215 | B |
| 216 | ND |
| 217 | A |
| 218 | B |
| 219 | A |
| 220 | B |
| 221 | A |
| 222 | B |
| 223 | A |
| 224 | A |
| 225 | A |
| 226 | B |
| 227 | A |
| 228 | B |
| 229 | B |
| 230 | B |
| 231 | A |
| 232 | B |
| 233 | C |
| 234 | A |
| 235 | A |
| 236 | B |
| 237 | B |
| 238 | A |
| 239 | B |
| 240 | B |
| 241 | B |
| 242 | A |
| 243 | A |
| 244 | B |
| 245 | B |
| 246 | A |
| 247 | B |
| 248 | A |
| 249 | A |
| 250 | A |
| 251 | A |
| 252 | A |
| 253 | A |
| 254 | A |
| 255 | A |
| 256 | A |
| 257 | A |
| 258 | B |
| 259 | B |
| 260 | A |
| 261 | B |
| 262 | A |
| 263 | B |
| 264 | A |
| 265 | A |
| 266 | A |
| 267 | B |
| 268 | B |
| 269 | A |
| 270 | A |
| 271 | B |
| 272 | B |
| 273 | A |
| 274 | A |
| 275 | B |
| 276 | B |
| 277 | B |
| 278 | B |
| 279 | B |
| 280 | A |
| 281 | A |
| 282 | B |
| 283 | A |
| 284 | B |
| 285 | B |
| 286 | B |
| 287 | A |
| 288 | B |
| 289 | A |
| 290 | A |
| 291 | A |
| 292 | B |
| 293 | C |
| 294 | B |
| 295 | C |
| 296 | B |
| 297 | C |
| 298 | B |
| 299 | B |
| 300 | B |
| 301 | B |
| 302 | A |
| 303 | A |
| 304 | B |
| 305 | C |
| 306 | A |
| 307 | C |
| 308 | B |
| 309 | C |
| 310 | B |
| 311 | B |
| 312 | C |
| 313 | C |
| 314 | B |
| 315 | B |
| 316 | B |
| 317 | A |
| 318 | B |
| 319 | A |
| 320 | B |
| 321 | B |
| 322 | B |
| 323 | B |
| 324 | B |
| 325 | B |
| 326 | A |
| 327 | A |
| 328 | A |
| 329 | B |
| 330 | A |
| 331 | A |
| 332 | B |
| 333 | C |
| 334 | B |
| 335 | B |
| 336 | A |
| 337 | ND |
| 338 | B |
| 339 | B |
| 340 | B |
| 341 | B |
| 342 | C |
| 343 | B |
| 344 | B |
| 345 | B |
| 346 | B |
| 347 | B |
| 348 | B |
| 349 (Reference | A |
| Compound A) | |
| 351 | B |
| 352 | A |
| 353 | A |
| 354 | A |
| 354-a | D |
| 355 | A |
| 356 | A |
| 356-a | C |
| 357 | A |
| 358 | B |
| 359 | B |
| 360 | B |
| 363 | A |
| 365 | B |
| 366 | B |
| 366-a | C |
| 368 | A |
| 368-a | D |
| 369 | A |
| 370 | A |
| 371 | C |
| 371-a | D |
| 373 | A |
| 374 | A |
| 374-a | B |
| 375 | B |
| 376 | A |
| 379 | A |
| 380 | B |
| 381 | A |
| 382 | A |
| 383 | A |
| 384 | A |
| 385 | A |
| 385-a | C |
| 398 | A |
| 401 | A |
| 402 | A |
| 403 | B |
| 406 | B |
| 407 | A |
| 408 | A |
| 409 | |
| 414 | A |
| 415 | A |
| 418 | A |
| 419 | C |
| 420 | A |
| 422 | A |
| 428 | A |
| 434 | A |
| 435 | A |
| 436 | B |
| 437 | B |

### EXAMPLE 91. In vitro hERG assay protocol

### Cell Culture

CHO cells stably expressing hERG potassium channels from Sophion Biosciences were used for this test. The cells were cultured in a humidified and air-controlled (5% CO₂) incubator at 37° C.

The CHO hERG Culture Medium consisted of 500 mL Ham's F12 (Invitrogen), 50 mL FBS (HyClone), 1 mL G418/Geneticin (Invitrogen, and 1 mL Hygromycin B (Invitrogen)

### Preparation of Cells

The CHO cells which were at least two days after plating and more than 75% confluent would be used for experiments. Before testing, cells were harvested using TrypLE and resuspended in the physiological solution at the room temperature.

### Recording Solutions

For the electrophysiological recordings the following solutions were used (Table 5). The physiological solution and external solution were prepared at least one month. The intracellular solution was prepared in batches aliquoted, and stored at 4° C. until used.

TABLE 5
Composition of Physiological, External and Internal Solutions

| | Physiological | External | Internal |
|---|---|---|---|
| Reagent | Solution (mM) | Solution(mM) | Solution(mM) |
| NaCl | 140 | 80 | 10 |
| KCl | 4 | 4 | 10 |
| KF | — | — | 110 |
| CaCl₂ | 2 | 2 | — |
| MgCl₂ | 1 | 1 | — |
| Glucose | 5 | 5 | — |
| NMDG | — | 60 | — |
| HEPES | 10 | 10 | 10 |
| EGTA | — | — | 10 |
| pH | 7.4 with NaOH | 7.4 with NaOH | 7.2 with KOH |
| Osmolarity | ~298 mOsm | ~289 mOsm | ~280 mOsm |

### Preparation of Compounds

Test compounds were dissolved in 100% DMSO to obtain stock solutions for different test concentrations. Then the stock solutions were further diluted into external solution to achieve final concentrations for testing. Visual check for precipitation was conducted before testing. Final DMSO concentration in external solution was not more than 0.30% for the test compounds.

### Voltage Protocol

Voltage command protocol: From this holding potential of −80 mV, the voltage was first stepped to −50 mV for 80 ms for leak subtraction, and then stepped to +20 mV for 4800 ms to open hERG channels. After that, the voltage was stepped back down to −50 mV for 5000 ms, causing a “rebound” or tail current, which was measured and collected for data analysis. Finally, the voltage was stepped back to the holding potential (−80 mV, 1000 ms). This voltage command protocol was repeated every 20000 msec. This command protocol was performed continuously during the test (vehicle control and test compound).

### SyncroPatch Whole-Cell Recording

hERG SyncroPatch assay was conducted at room temperature. The Setup, Prime Chip, Catch and Seal Cells, Amplifier Settings, Voltage and Application Protocols were established with Biomek Software (Nanion). One addition of 40 μL of the vehicle was applied, followed by 300s for a baseline period. Then the doses of the compounds were added with 40 μL. The exposure of test compound at each concentration was no less than 300s. The recording for the whole process had to pass the quality control or the well was abandoned and the compound was retested, all automatically set by PatchControl. Five concentrations (0.30 μM, 1.00 μM, 3.00 μM, 10.00 μM and 30.00 μM) were tested for each compound. Minimum 2 replicates per concentration were obtained.

### Data Analysis

Data analysis was carried out using DataControl, Excel 2013 (Microsoft) and GraphPad Prism 5.0. Within each well recording, percent of control values were calculated for each test compound concentration current response based on peak current in presence of reference control (current response/peak current)×100%. The Dose-Response curves were fit to the standard Hill equation as shown below:

$${I}_{post cpd} / {I}_{pre cpd} = Bottom + ( Top - Bottom ) / ( 1 + 10 \hat{} ( ( Log {IC}_{50} - X ) * HillSlope ) )$$

Where X is the logarithm of concentration, I_{post cpd}/I_{pre cpa} is the normalized peak current amplitude, Top is 1 and Bottom is equal to 0.

Curve-fitting and IC₅₀ calculations were performed by GraphPad Prism 5.0. If the inhibition obtained at the lowest concentration tested was over 50%, or at the highest concentration tested was less than 50%, we reported the IC₅₀ as less than lowest concentration, or higher than highest concentration, respectively.

The hERG IC₅₀ determined for each compound using the assay is summarized in Table 6 below. The compound numbers correspond to those shown in Table 1.

In Table 6, for hERG IC₅₀ data, “A” indicates an IC₅₀ of less than 2 μM, “B” indicates an IC₅₀ range from 2 μM to 5 μM; “C” indicates an IC₅₀ range from 5 μM to 10 μM; and “D” indicates an IC₅₀ greater than 10 μM. For hERG/H3K9me2 ratio data, “A” indicates a ratio of less than 100, “B” indicates a ratio of at least 100 but less than 500, “C” indicates a ratio of at least 500 but less than 1000, and “D” indicates a ratio of 1000 or greater.

TABLE 6
hERG channel inhibition of exemplary
compounds of the invention

| | hERG | |
|---|---|---|
| Compound | IC₅₀ | hERG/H3K9me2 |
| No. | (μM) | ratio |
| Reference | C (retest | A (retest result) |
| Compound A | result) | |
| Reference | C | B |
| Compound B | | |
| 103 | D | A |
| 110 | D | D |
| 111 | D | B |
| 113 | A | A |
| 118 | C | A |
| 122 | B | A |
| 129 | B | B |
| 130 | B | B |
| 131 | C | A |
| 134 | D | A |
| 137 | C | B |
| 142 | A | A |
| 144 | D | — |
| 151 | D | — |
| 157 | D | — |
| 160 | C | C |
| 166 | A | B |
| 167 | C | B |
| 169 | C | B |
| 170 | A | B |
| 172 | B | A |
| 175 | C | C |
| 176 | B | B |
| 177 | C | B |
| (Reference | | |
| Compound B) | | |
| 179 | D | D |
| 180 | D | D |
| 183 | D | D |
| 187 | D | B |
| 188 | D | D |
| 189 | D | D |
| 190 | C | B |
| 191 | B | B |
| 193 | C | C |
| 201 | C | C |
| 289 | D | C |
| 290 | D | C |
| 291 | D | D |
| 322 | B | A |
| 323 | B | A |
| 324 | C | A |
| 325 | B | A |
| 326 | B | A |
| 327 | D | B |
| 328 | D | D |
| 329 | D | A |
| 330 | D | B |
| 331 | D | B |
| 332 | D | A |
| 334 | D | A |
| 335 | C | A |
| 336 | C | B |
| 341 | D | B |
| 348 | D | B |
| 349 | C | A |
| (Reference | | |
| Compound A) | | |
| 357 | C | D |
| 369 | C | D |
| 370 | D | D |
| 373 | D | D |
| 374 | D | C |
| 381 | D | D |
| 384 | D | D |

Reference Compounds A and B have the structures:

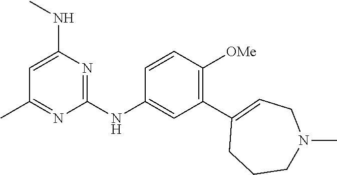

Reference Compound A (Compound 349); hERG IC50: C, hERG/H3K9me2 ratio: A. Note that the hERG values for this compound are retest results which replace previously disclosed values.

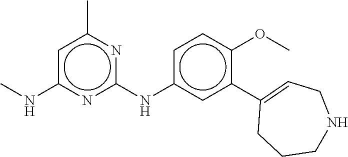

Reference Compound B (Compound 177)); hERG IC50: C, hERG/H3K9me2 ratio: B

Reference A can be compared to present compound 111.

Compound 111

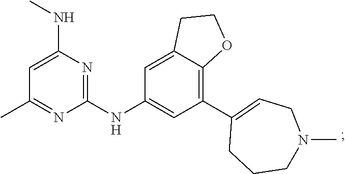

hERG IC50: D, hERG/H3K9me2 ratio: B Reference B can be compared to present compounds 110 and 291.

Compound 110

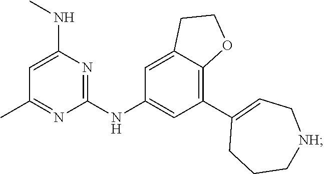 [SMILES: CNc1cc(C)nc(Nc2cc3c(c(C4=CCNCCC4)c2)OCC3)n1]

hERG IC50: D, hERG/H3K9me2 ratio: D

Compound 291

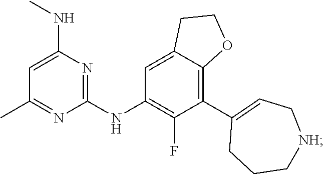 [SMILES: CNc1cc(C)nc(Nc2cc3c(c(C4=CCNCCC4)c2F)OCC3)n1]

hERG IC50: D, hERG/H3K9me2 ratio: D

It can be noted that higher hERG IC₅₀ is indicative of lower likelihood of cardiac adverse effects. The ratio of hERG/H3K9me2 is also useful as an alternative measure as it corrects for the likely dose of the agent and is a measure of therapeutic index, with a higher ratio indicative of improved therapeutic index regarding cardiac toxicity. Thus the comparison of compounds 110, 111, and 291 with reference compounds above suggests that some compounds of the present disclosure have lower risk of hERG-mediated cardiac adverse effects compared to compounds in the series of Reference Compounds A and B (which lack an oxygen-containing ring).

### EQUIVALENTS

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

We claim:

1. A compound of formula (I) or a pharmaceutically acceptable salt thereof 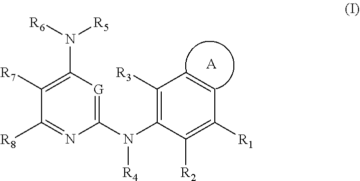
wherein,
A is an optionally substituted 5- or 6-membered oxygen-containing heterocyclic ring;
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by O or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted;
R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, N(R¹²)₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂-C₁-C₆ alkyl; SO_{w}—C₁-C₆ alkyl; CO₂-C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

2. A compound of formula (II), or a pharmaceutically acceptable salt thereof 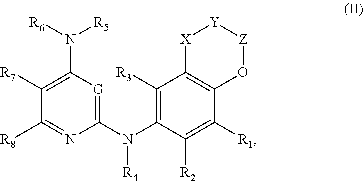
wherein X is C(R¹¹)₂, O, S(O)_{w}, or NR¹²;
Y is a bond, C(R¹³)₂, or C(R¹³)₂—C(R¹³)₂; and
Z is CR⁹R¹⁰;
or X—Y is C(R¹¹)═C(R¹³) and Z is CR⁹R¹⁰; or
X—Y—Z is C(R¹¹)═C(R¹³);
G is N or CR⁷;
R¹ is —O—C₁-C₆ alkylene-E, —NR¹²—C₁-C₆ alkylene-E, or E, wherein each methylene group in C₁-C₆ alkylene is individually optionally replaced by 0 or NR′, and wherein C₁-C₆ alkylene is optionally substituted with 1-3 individually selected halo or C₁-C₆ alkyl;
E is C₃-C₁₀ cycloalkyl or C₃-C₁₀ heterocycloalkyl, each of which is optionally substituted; R′ is H or C₁-C₆ alkyl;
each R² and R³ is independently H, C₁-C₆ alkyl, C₁-C₆ heteroalkyl, phenyl, C₃-C₇ cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
R⁴ is H, C₁-C₆ alkyl or C₁-C₆ heteroalkyl;
each R⁵ and R⁶ is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
R⁵ and R⁶ are taken together with the nitrogen to which they are attached to form a 3-7 membered heterocycle with 0-2 additional ring heteroatoms selected from O, S, and N, and wherein the heterocycle is optionally substituted;
each R⁷ and R⁸ is independently H, C₁-C₆ alkyl, C₁-C₆ heteroalkyl, phenyl, cycloalkyl, heterocyclyl, C₁-C₆ alkoxy, hydroxy, cyano, CO—R^{C}, NR^{D}₂, or halogen, wherein each alkyl, heteroalkyl, phenyl, cycloalkyl, or heterocyclyl is optionally substituted;
each R⁹ and R¹⁰ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted;
each R¹¹ and R¹³ is independently H, C₁-C₆ alkyl, C₁-C₆ heteroalkyl, phenyl, cycloalkyl, heterocyclyl, hydroxy, cyano, CO—R^{C}, NR^{D}₂, or halogen, wherein each alkyl, heteroalkyl, phenyl, cycloalkyl, or heterocyclyl is optionally substituted; or
two R¹¹ or two R¹³ can be taken together with the carbon to which they are attached to form CO or a spirofused C₃-C₇ cycloalkyl;
each R¹² is independently H, C₁-C₆ alkyl, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R¹² attached to the same nitrogen are taken together with the nitrogen to which they are attached to form a 3-7 membered heterocycle with 0-2 additional ring heteroatoms selected from O, S, and N, and wherein the heterocycle is optionally substituted;
each R^{C} is independently H, OH, NR¹²₂, C₁-C₆ alkyl, C₁-C₆ alkoxy, C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted;
each R^{D} is independently H, C₁-C₆ alkyl, CO—C₁-C₆ alkyl; CO₂-C₁-C₆ alkyl; SO_{w}—C₁-C₆ alkyl; CO₂-C₁-C₆ alkyl; C₁-C₆ heteroalkyl, phenyl, C₃-C₇ cycloalkyl, or heterocyclyl, wherein each alkyl, heteroalkyl, phenyl, cycloalkyl, or heterocyclyl is optionally substituted; or
two R^{D} attached to the same nitrogen are taken together with the nitrogen to which they are attached to form a 3-7 membered heterocycle with 0-2 additional ring heteroatoms selected from O, S, and N, and wherein the heterocycle is optionally substituted; and
w is 0, 1, or 2.

3. The compound of any claim 1 or 2, or a pharmaceutically acceptable salt thereof, wherein G is CH.

4. A compound of formula (III), or a pharmaceutically acceptable salt thereof 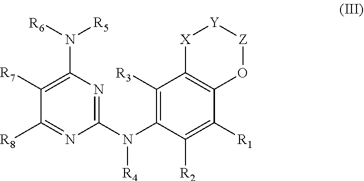
wherein
X is C(R¹¹)₂, O, or NR¹²;
Y is a bond or C(R¹³)₂, and
Z is CR⁹R¹⁰; or
X-Y is C(R¹¹)═C(R¹³) and Z is CR⁹R¹⁰; or
X—Y—Z is C(R¹¹)═C(R¹³);
R¹ is —O—C₁-C₆alkylene-E, —NR¹²—C₁-C₆ alkylene-E, or E;
E is C₃-C₁₀ cycloalkyl or heterocycloalkyl, each of which is optionally substituted with 1-4R^{E};
each R² and R³ is independently H, C₁-C₆ alkyl, C₁-C₆ haloalkyl, C₁-C₆ heteroalkyl, C₁-C₆ alkoxy, hydroxy, cyano, or halogen;
R⁴ is H or C₁-C₆ alkyl;
each R⁵ and R⁶ is independently H, C₁-C₆ alkyl, C₁-C₆ heteroalkyl, phenyl, C₃-C₇ cycloalkyl, heterocyclyl, C₁-C₆ alkylene-phenyl, C₁-C₆ alkylene-C₃-C₇ cycloalkyl, or C₁-C₆ alkylene-heterocyclyl, wherein each alkyl or heteroalkyl is optionally substituted with 1-5 substituents independently selected from deuterium, halogen and OH, and wherein each phenyl, cycloalkyl, or heterocyclyl is optionally substituted with 1-4 R^{E}; or
R⁵ and R⁶ are taken together with the nitrogen to which they are attached to form a 3-7 membered heterocycle with 0-2 additional ring heteroatoms selected from O, S, and N, and wherein the heterocycle is optionally substituted with 1-4 R^{E};
each R⁷ and R⁸ is independently H, C₁-C₆ alkyl, C₁-C₆ alkoxy, C₁-C₆ haloalkyl, C₁-C₆ heteroalkyl, hydroxy, cyano, or halogen, wherein C₁-C₆ alkyl is optionally substituted by one or more deuteriums;
each R⁹ and R¹⁰ is independently H, C₁-C₆ alkyl, C₁-C₆ haloalkyl, C₁-C₆ heteroalkyl, hydroxy, cyano, or halogen;
or R⁹ and R¹⁰ can be taken together with the carbon to which they are attached to form CO;
each R¹¹ and R¹³ is independently H, C₁-C₆ alkyl, C₁-C₆ haloalkyl, C₁-C₆ heteroalkyl, hydroxy, cyano, or halogen;
each R¹² is independently H, C₁-C₆ alkyl, C₁-C₆ haloalkyl, or C₁-C₆ heteroalkyl;
each R^{E} is independently selected from halogen, C₁-C₆ alkyl, C₁-C₆ haloalkyl, C₁-C₆ heteroalkyl, C₁-C₆ alkoxy, C₁-C₆ hydroxyalkyl, NH₂ and OH, wherein R^{E} is optionally substituted by one or more deuteriums.

5. The compound of any one of claims 2-4, or a pharmaceutically acceptable salt thereof, wherein X is CH(CH₃), CH₂ or O.

6. The compound of any one of claims 2-5, or a pharmaceutically acceptable salt thereof, wherein Y is a bond or CH₂.

7. The compound of any one of claims 1-6, or a pharmaceutically acceptable salt thereof, wherein R¹ is selected from E and —O—C₁-C₆ alkylene-E.

8. The compound of any one of claims 1-6, or a pharmaceutically acceptable salt thereof, wherein R¹ is E.

9. The compound of any one of claims 1-8, or a pharmaceutically acceptable salt thereof, wherein E is C₃-C₁₀ heterocycloalkyl optionally substituted with 1-4 R^{E}.

10. The compound of any one of claims 1-8, or a pharmaceutically acceptable salt thereof, wherein E is selected from 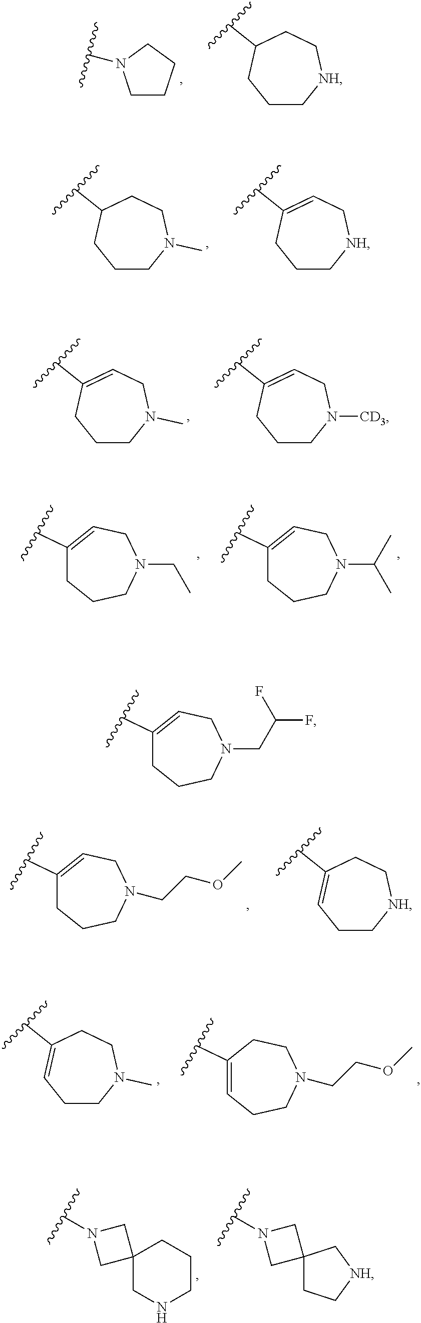 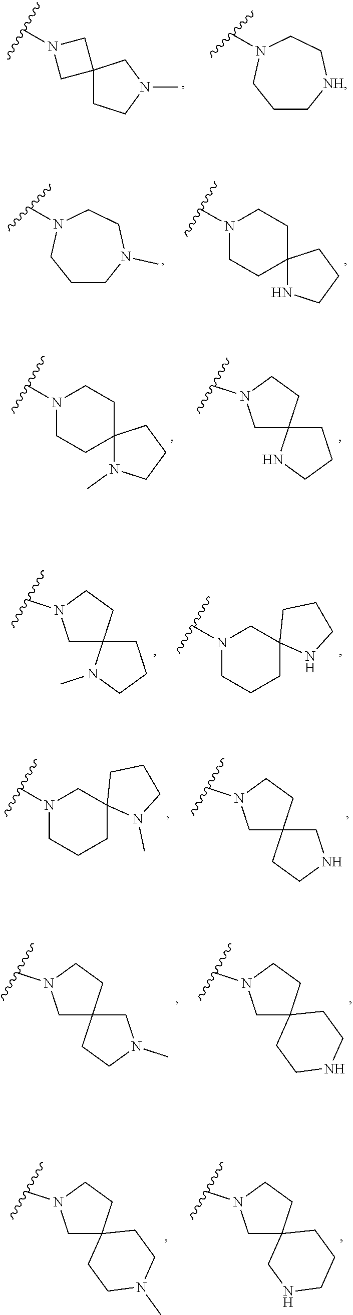 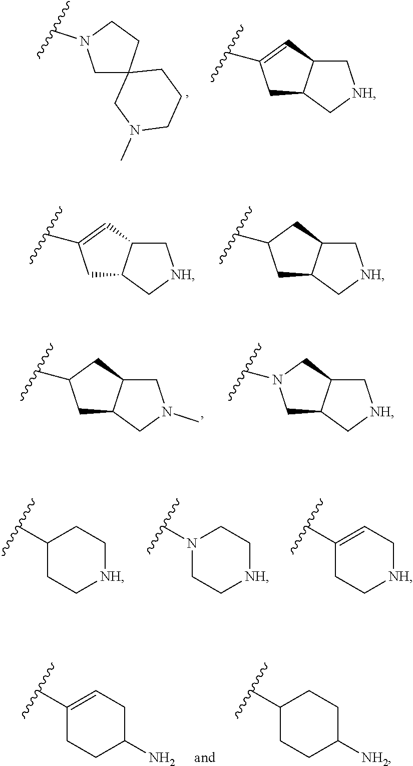
optionally substituted with 1-3 R^{E}.

11. The compound of any one of claims 1-8, or a pharmaceutically acceptable salt thereof, wherein E is 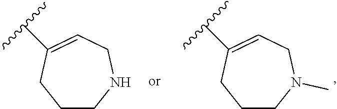
optionally substituted with 1-3 R^{E}.

12. The compound of any one of claims 1-11, or a pharmaceutically acceptable salt thereof, wherein each R^{E} is independently selected from halogen, C₁-C₆ alkyl, C₁-C₆ haloalkyl, C₁-C₆ alkoxy, C₁-C₆ heteroalkyl, C₁-C₆ hydroxyalkyl, NH₂ and OH, wherein R^{E} is optionally substituted with 1-5 deuteriums.

13. The compound of any one of claims 1-11, or a pharmaceutically acceptable salt thereof, wherein each R^{E} is independently selected from Me, CD₃, Et, F and OH.

14. The compound of any one of claims 1-13, or a pharmaceutically acceptable salt thereof, wherein R¹ is selected from

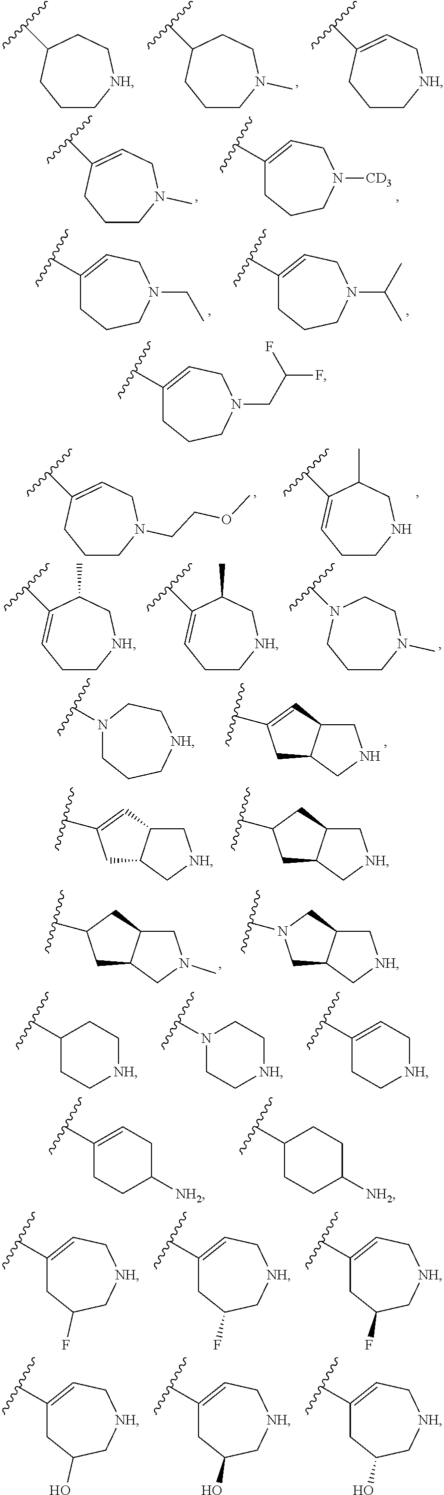

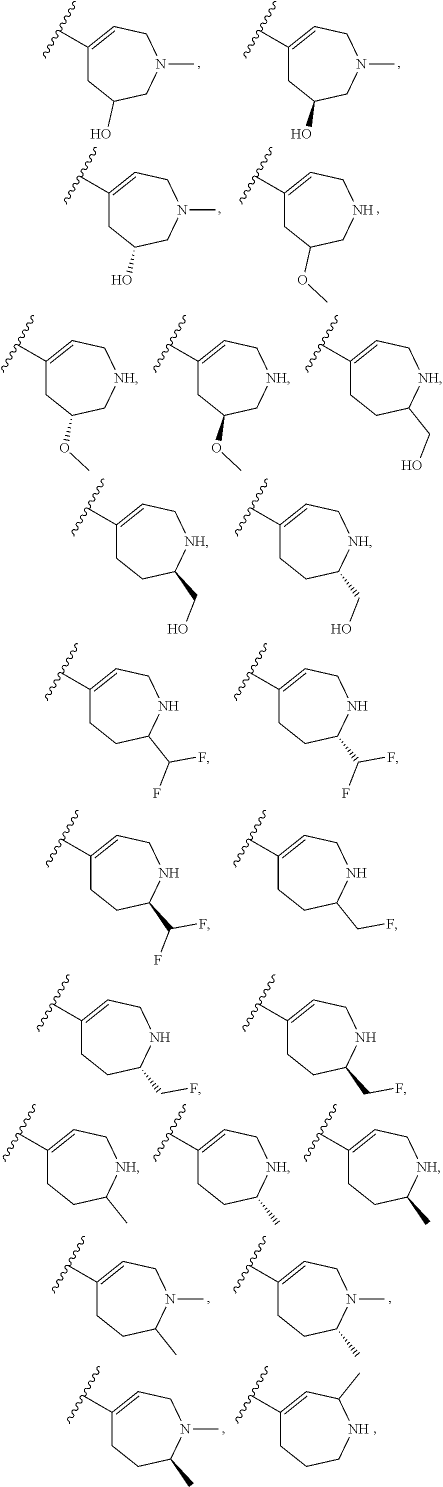

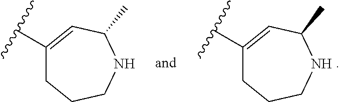

15. The compound of any one of claims 1-13, or a pharmaceutically acceptable salt thereof, wherein R¹ is selected from

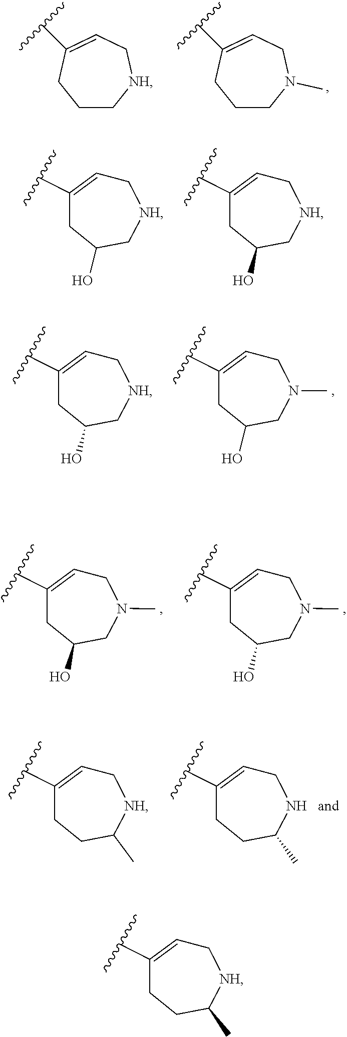

16. The compound of any one of claims 1-15, or a pharmaceutically acceptable salt thereof, wherein R² is selected from H, Me, F and C₁.

17. The compound of any one of claims 1-15, or a pharmaceutically acceptable salt thereof, wherein R² is selected from Me, F and C₁.

18. The compound of any one of claims 1-17, or a pharmaceutically acceptable salt thereof, wherein R³ is H.

19. The compound of any one of claims 1-18, or a pharmaceutically acceptable salt thereof, wherein R⁴ is H.

20. The compound of any one of claims 1-19, or a pharmaceutically acceptable salt thereof, wherein R⁵ is H.

21. The compound of any one of claims 1-20, or a pharmaceutically acceptable salt thereof, wherein R⁶ is selected from Me and CD₃.

22. The compound of any one of claims 1-21, or a pharmaceutically acceptable salt thereof, wherein R⁷ is selected from H, F and Me.

23. The compound of any one of claims 1-22, or a pharmaceutically acceptable salt thereof, wherein R⁸ is selected from methyl and CH₂D.

24. The compound of any one of claims 2-23, or a pharmaceutically acceptable salt thereof, wherein Z is selected from C═O, CF₂ and CH₂.

25. The compound of any one of claims 1-24, or a pharmaceutically acceptable salt thereof, wherein each R¹² is H.

26. The compound of any one of claims 2-25, or a pharmaceutically acceptable salt thereof, wherein each R¹¹ is independently selected from H, Me, hydroxy, and F, or two R¹¹ are taken together with the carbon to which they are attached to form a spirofused cyclopropyl.

27. The compound of any one of claims 2-26, or a pharmaceutically acceptable salt thereof, wherein each R¹³ is independently H.

28. A compound of formula (IVa-2) or a pharmaceutically acceptable salt thereof, wherein 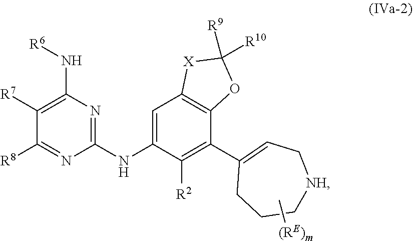
X is O or C(R¹¹)₂;
R² is selected from H, C₁-C₆ alkyl and halogen;
R⁶ is selected from C₁-C₆ alkyl and C₁-C₆ heteroalkyl, wherein each alkyl or heteroalkyl is optionally substituted with 1-5 instances of deuterium;
R⁷ is selected from H, C₁-C₆ alkyl, C₁-C₆ alkoxy, and halogen, wherein each alkyl is optionally substituted with 1-5 instances of deuterium;
R⁸ is C₁-C₆ alkyl, optionally substituted with 1-5 deuteriums;
each R⁹ and R¹⁰ is independently H, C₁-C₆ alkyl, or halogen;
each R¹¹ is independently selected from H, C₁-C₆ alkyl, hydroxy, and halogen, or two R¹¹ are taken together with the carbon to which they are attached to form a spirofused C₃-C₇ cycloalkyl
each R^{E} is independently selected from halogen, C₁-C₆ alkyl, C₁-C₆ haloalkyl, C₁-C₆ hydroxyalkyl and OH, wherein R^{E} is optionally substituted with 1-5 deuteriums; and
m is 0, 1, 2, 3 or 4.

29. The compound of any one of claims 1-28, or a pharmaceutically acceptable salt thereof, wherein the compound is selected from:

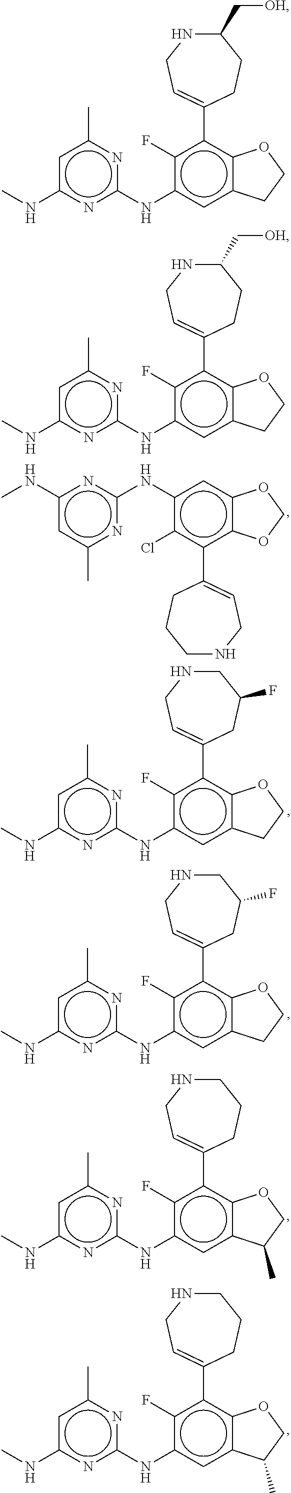

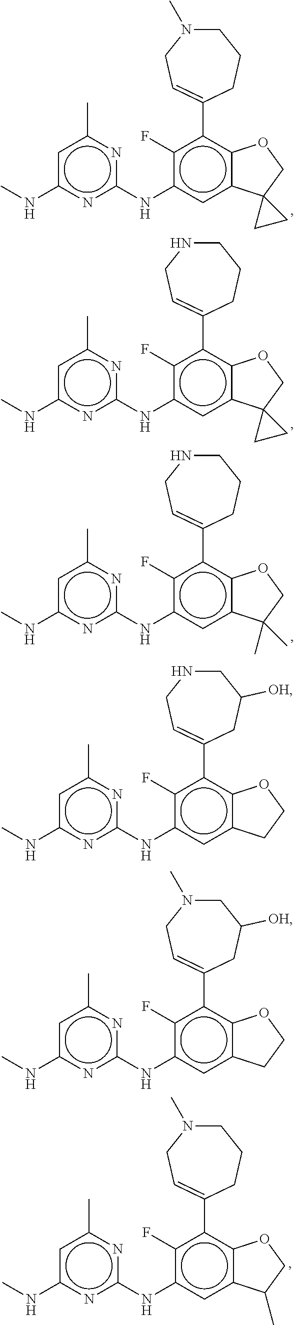

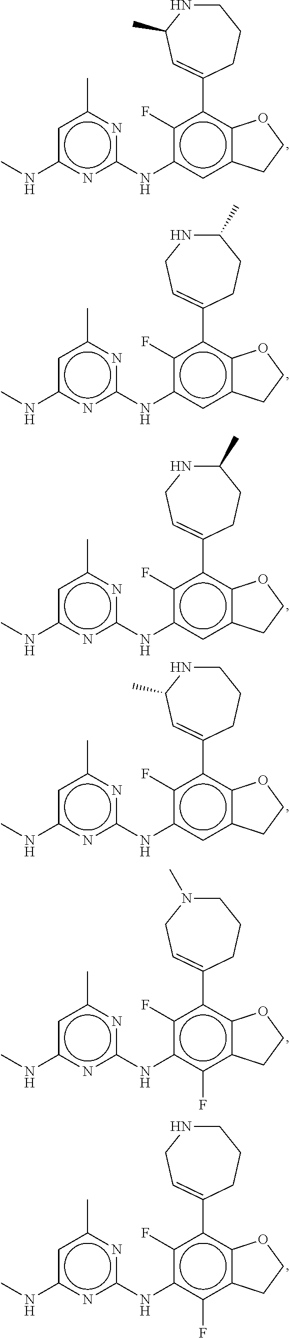

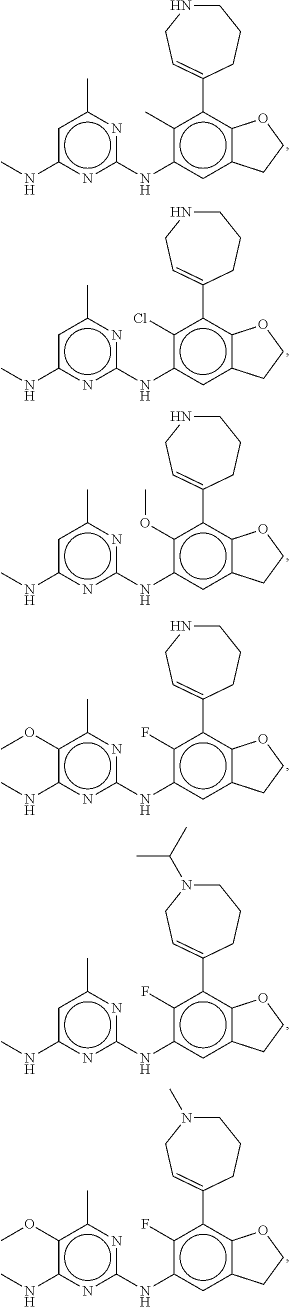

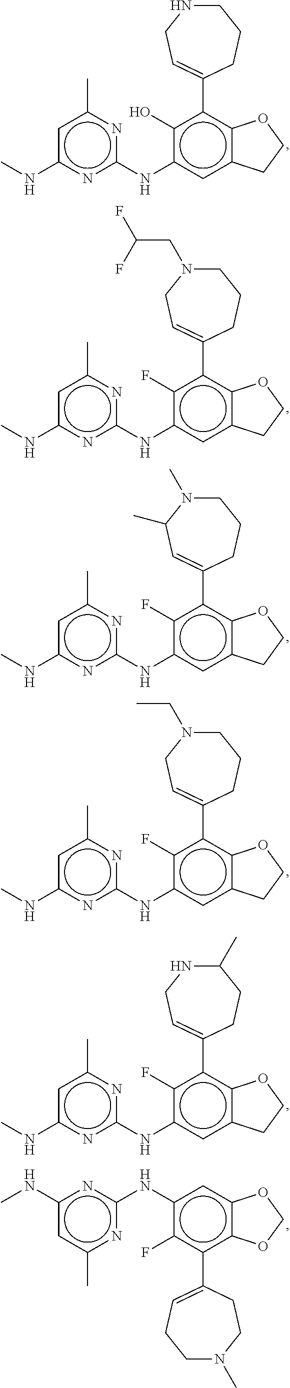

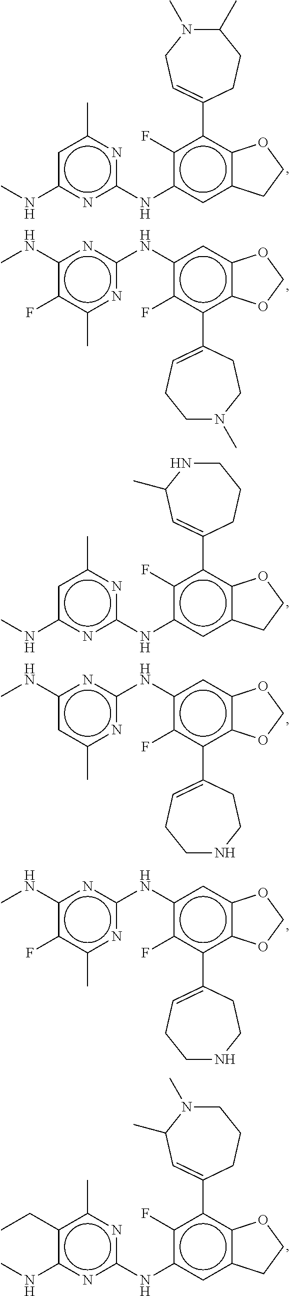

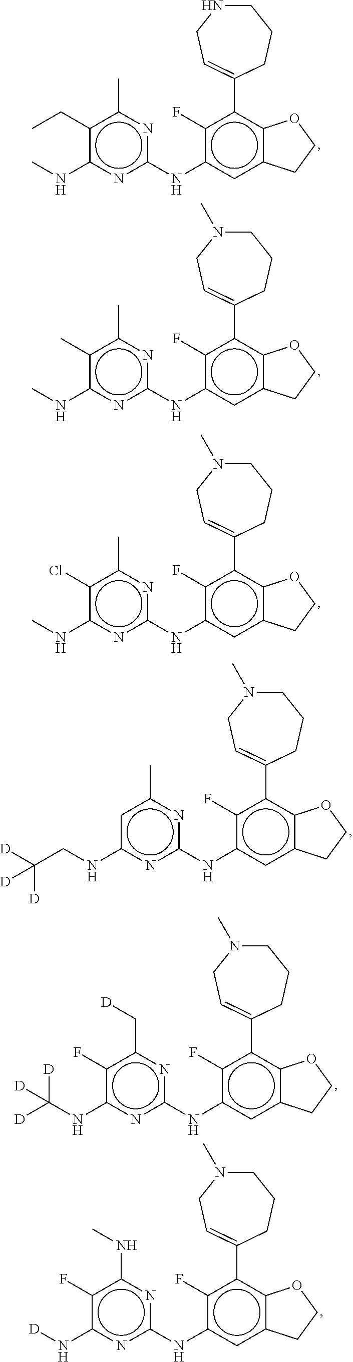

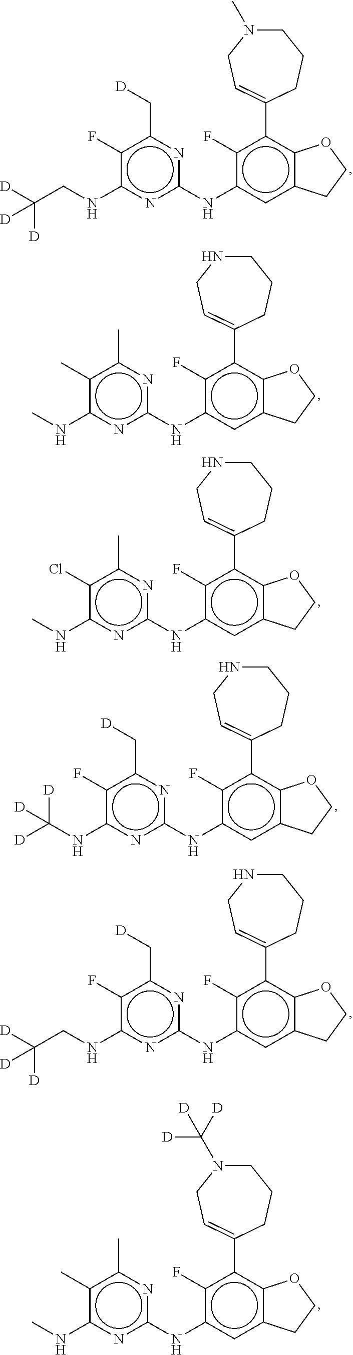

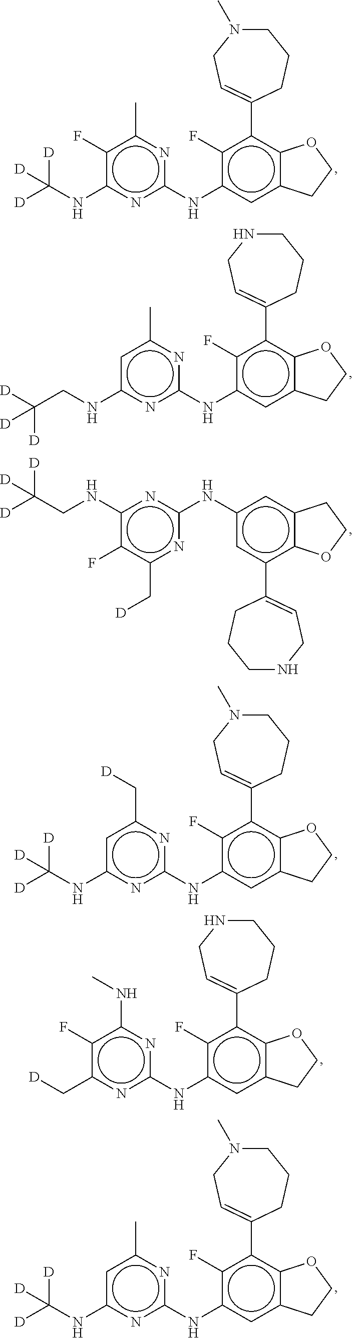

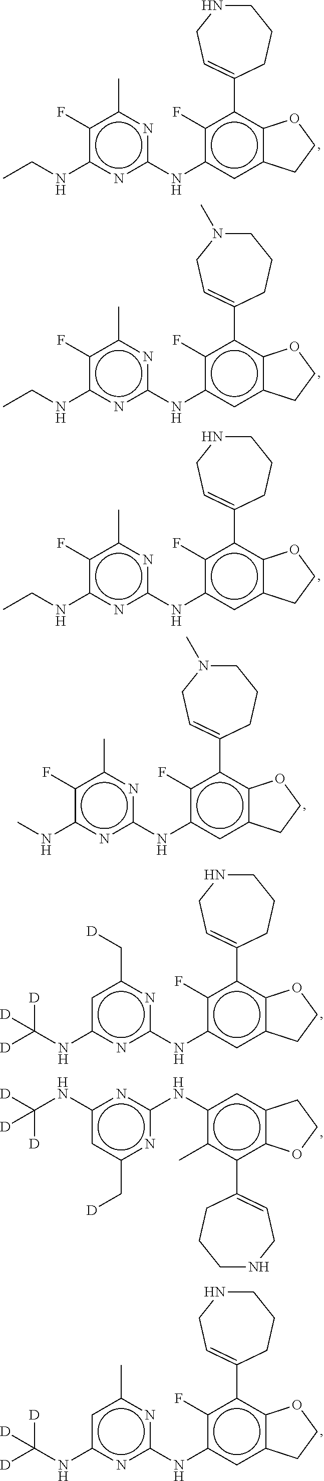

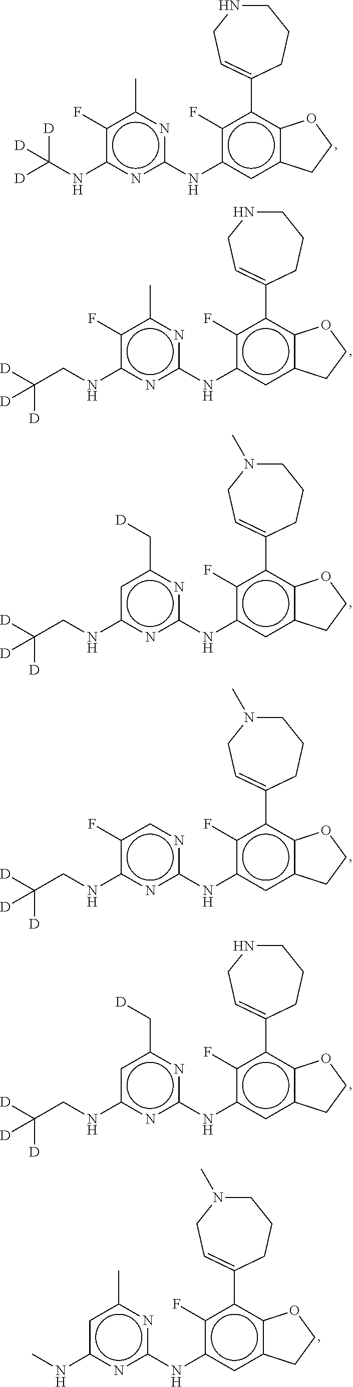

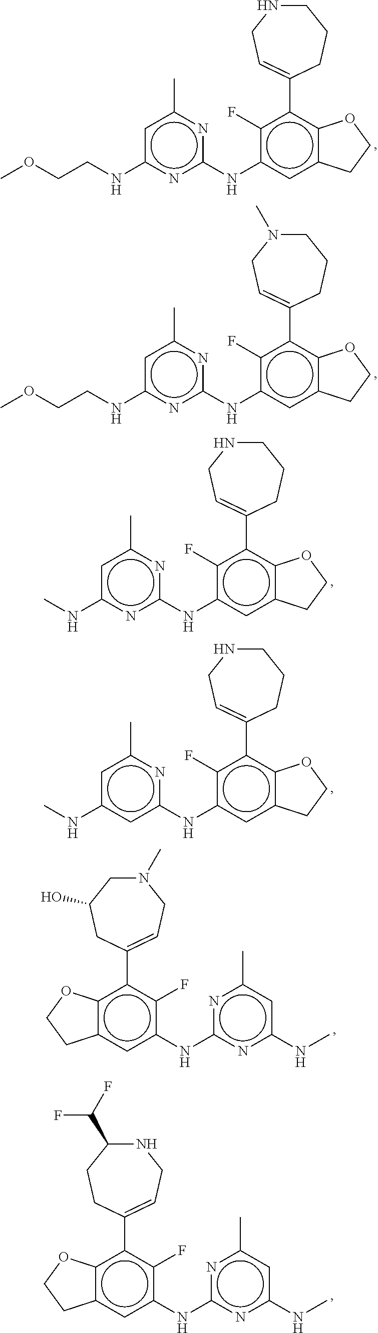

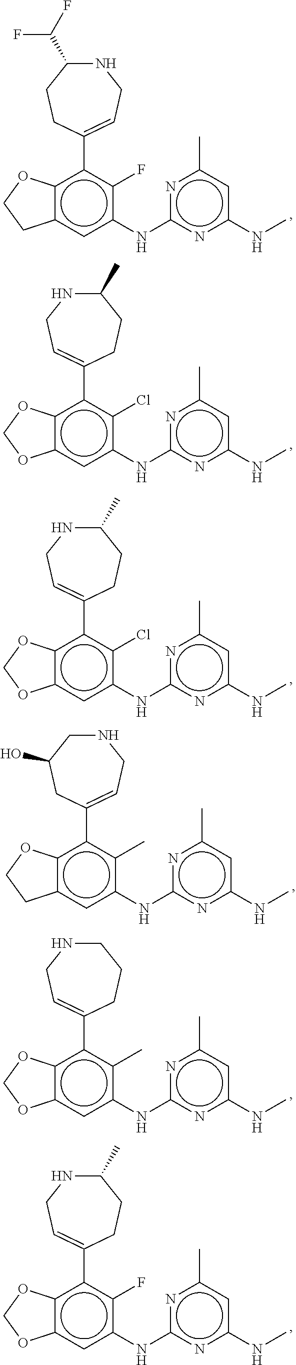

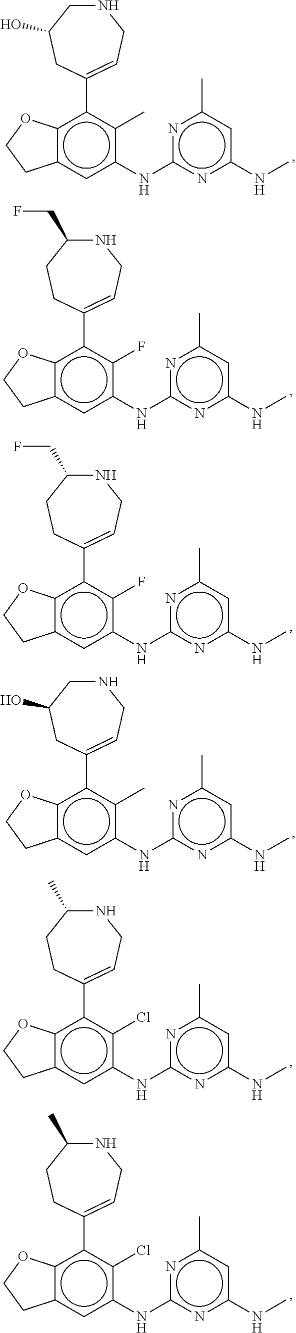

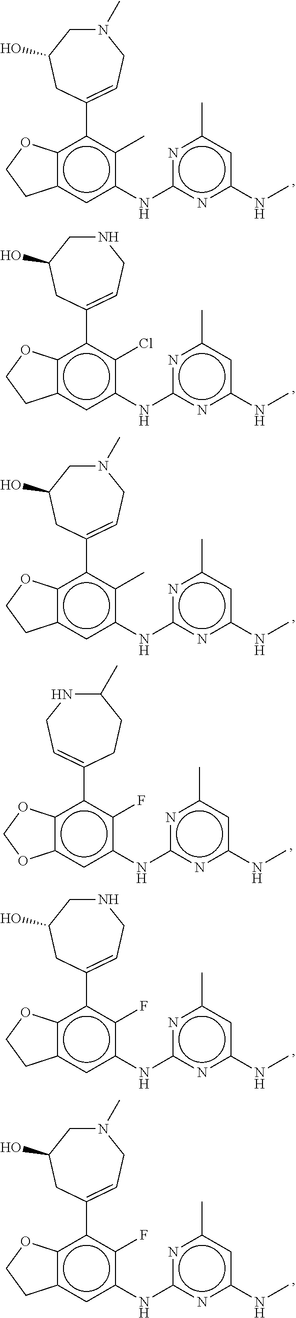

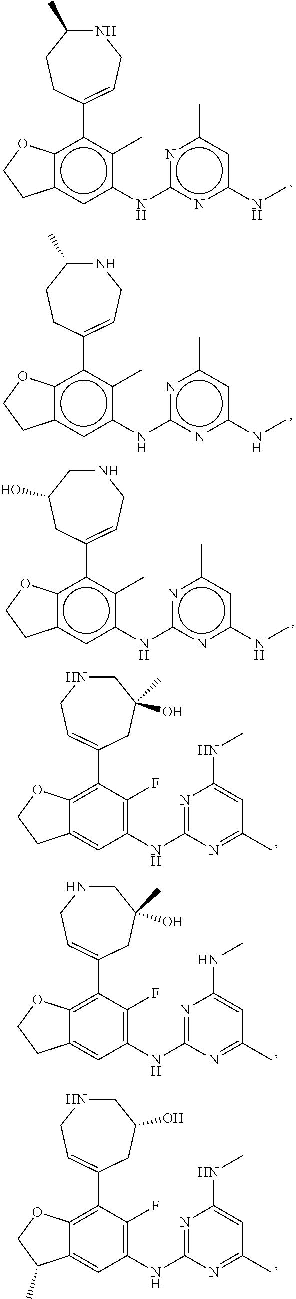

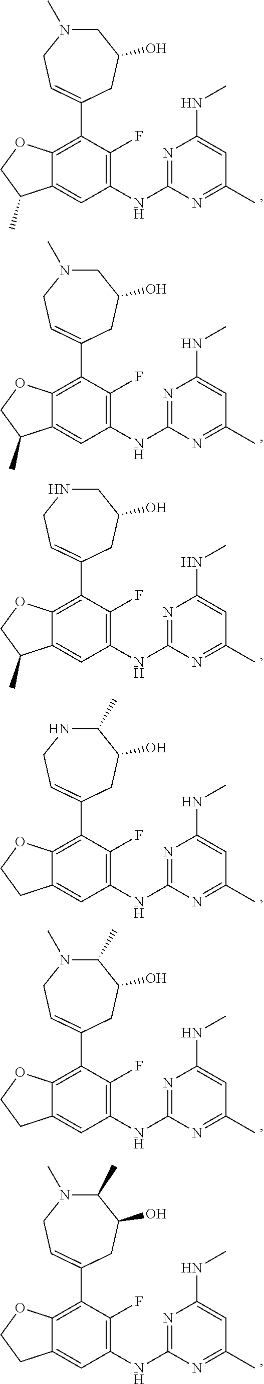

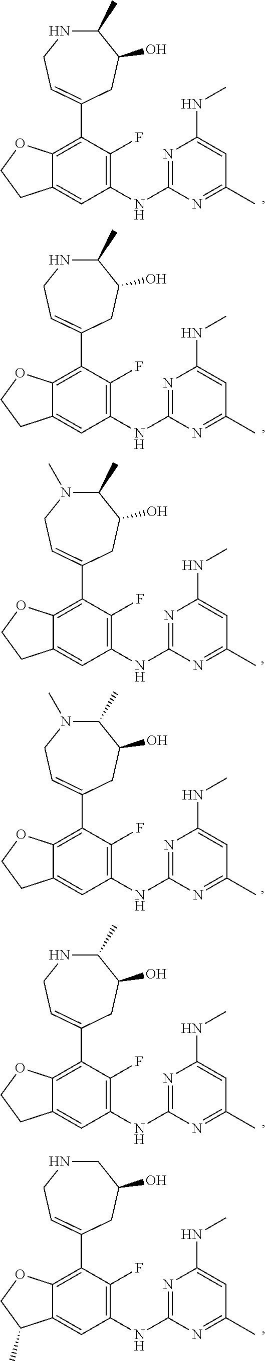

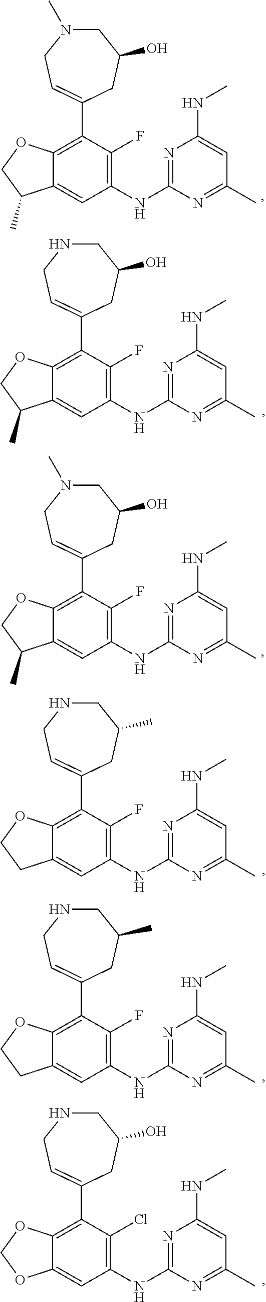

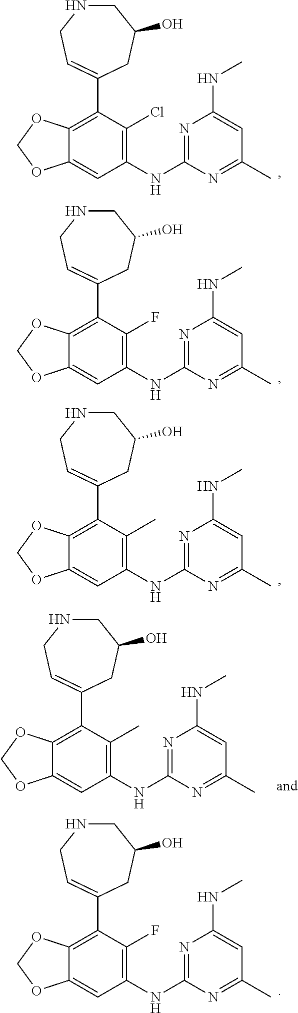

30. A compound of any one of claims 1 or 2, or a pharmaceutically acceptable salt thereof, wherein the compound is selected from

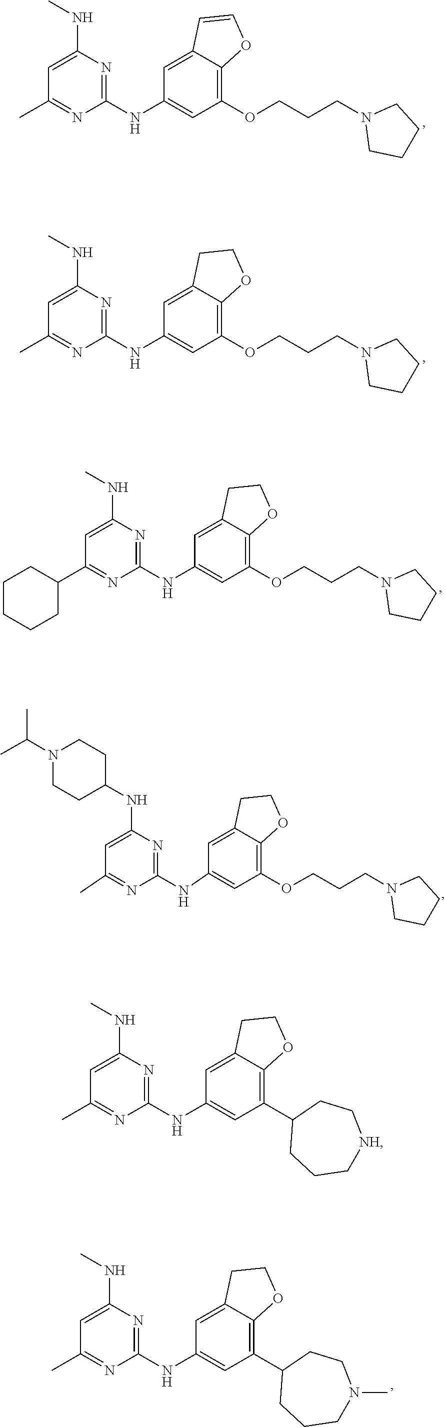

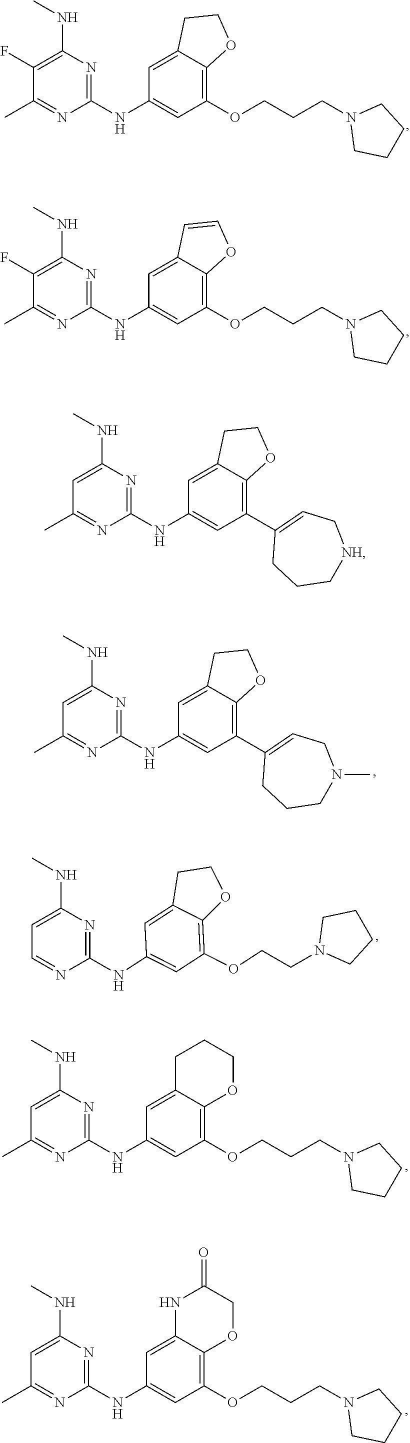

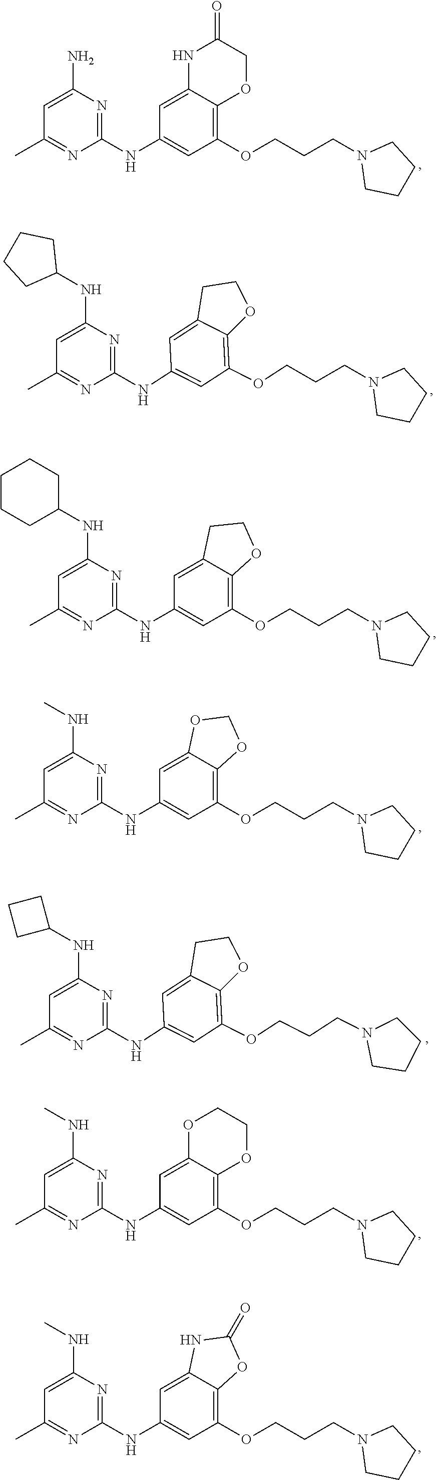

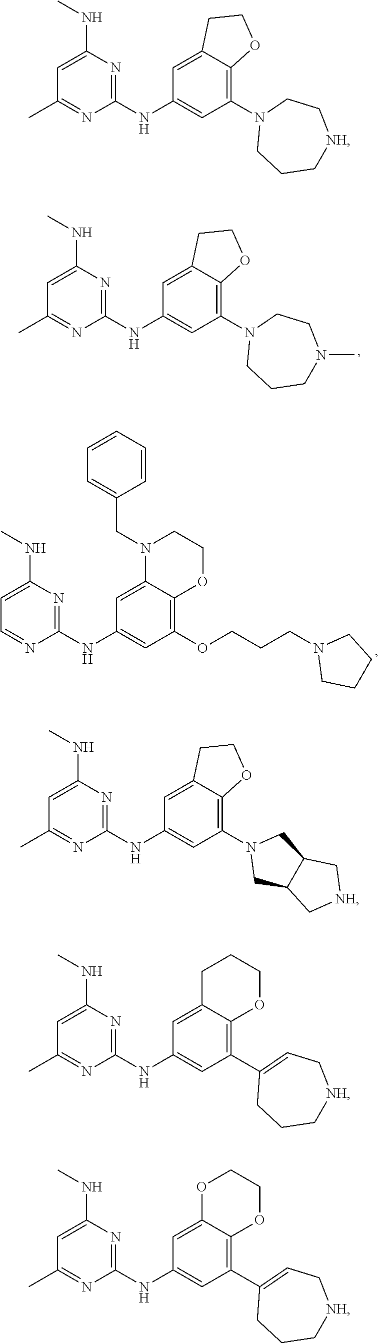

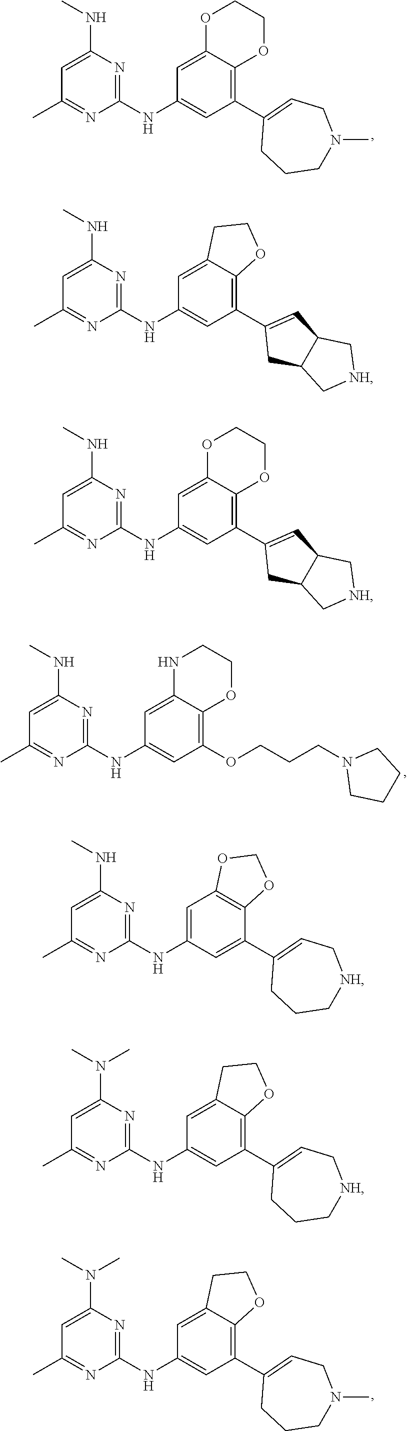

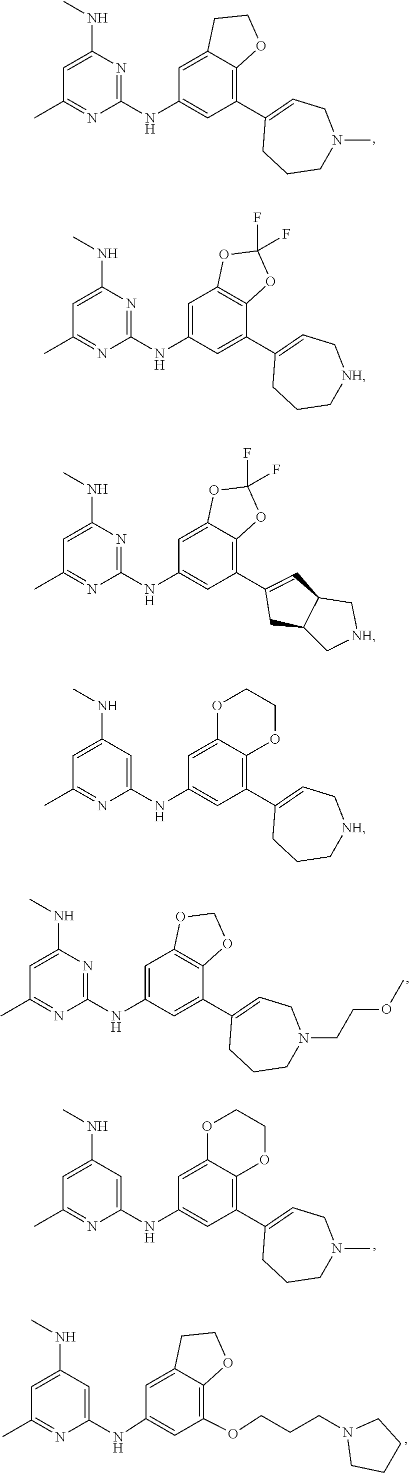

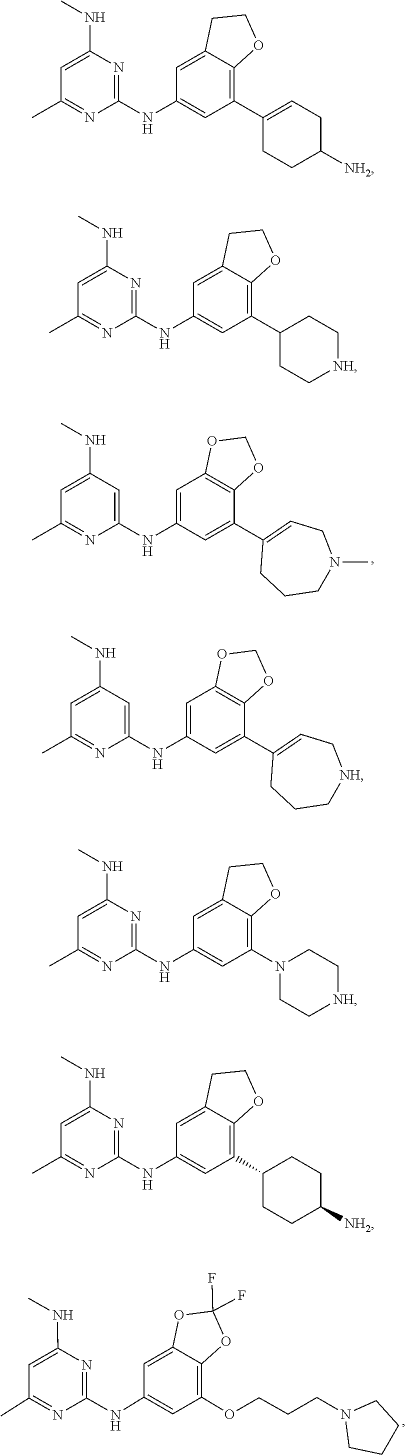

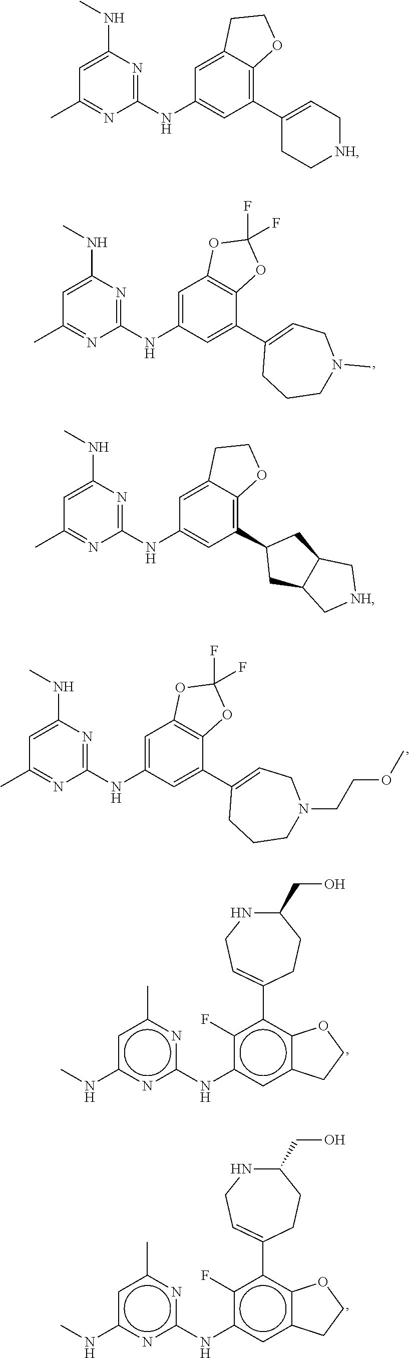

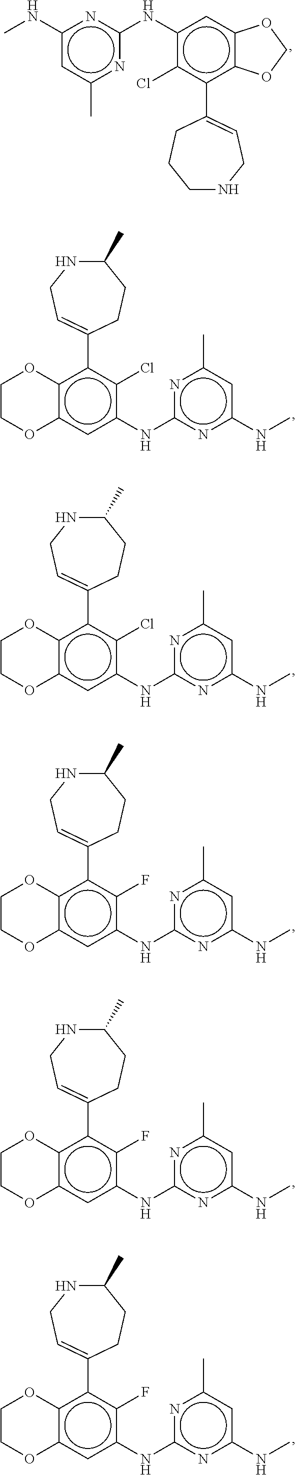

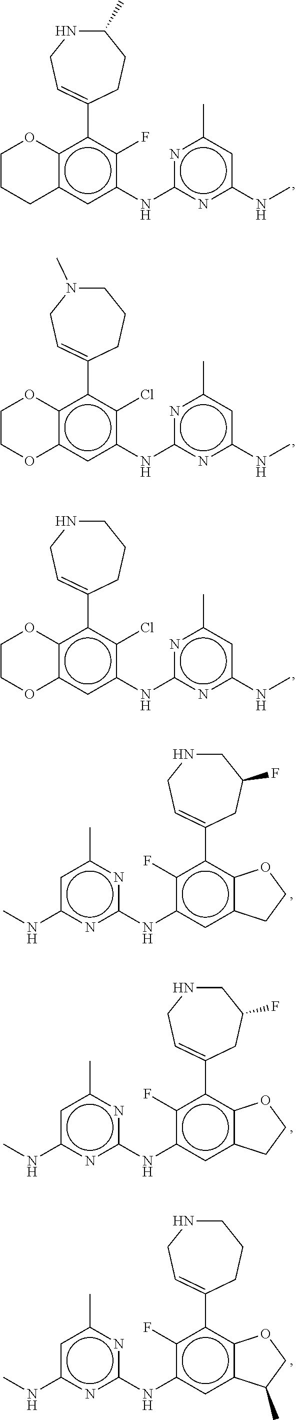

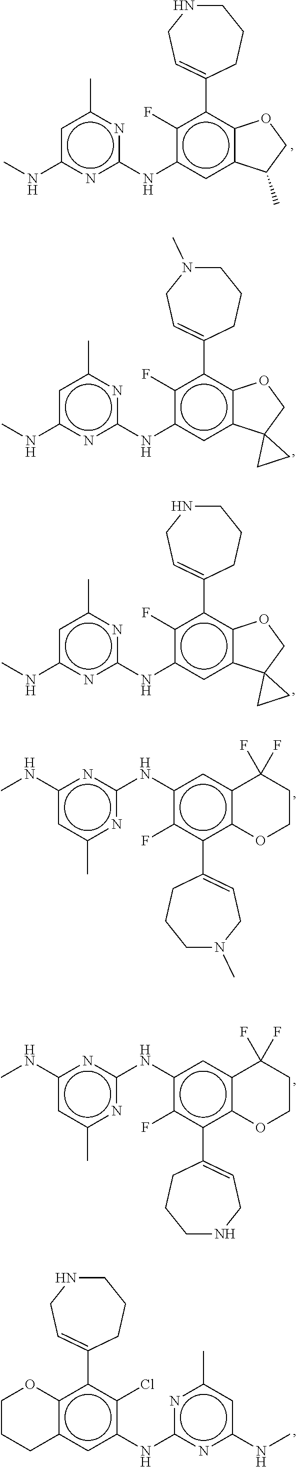

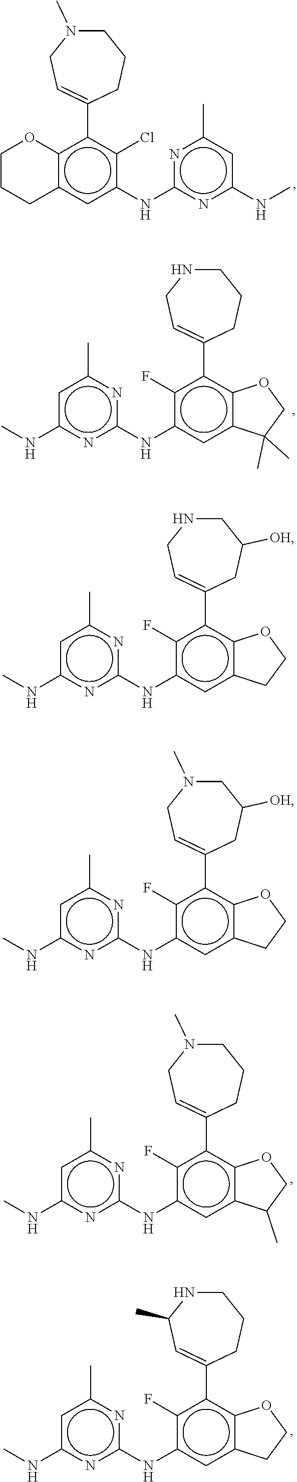

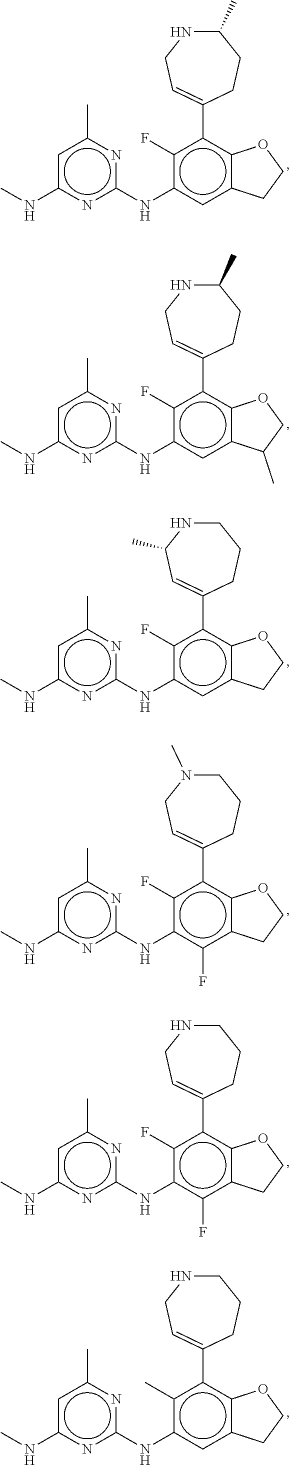

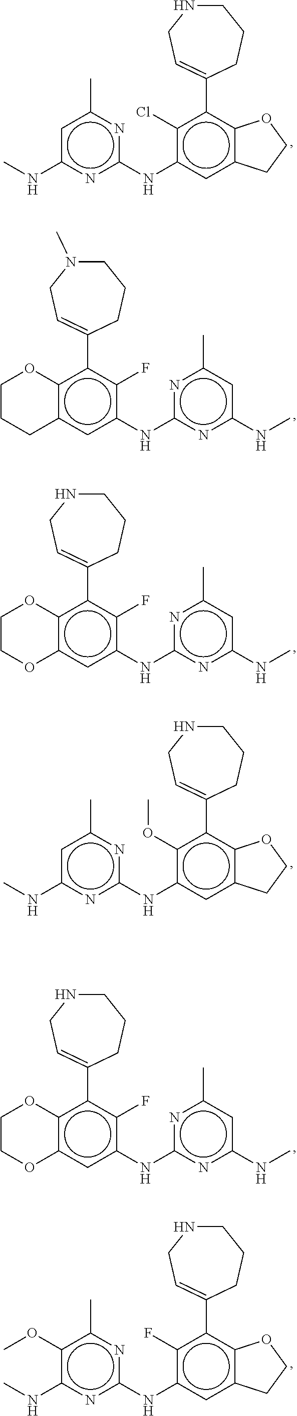

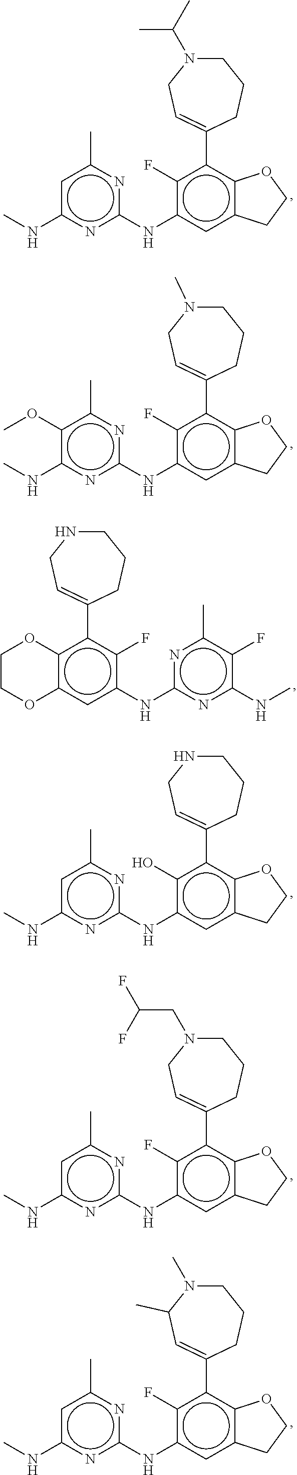

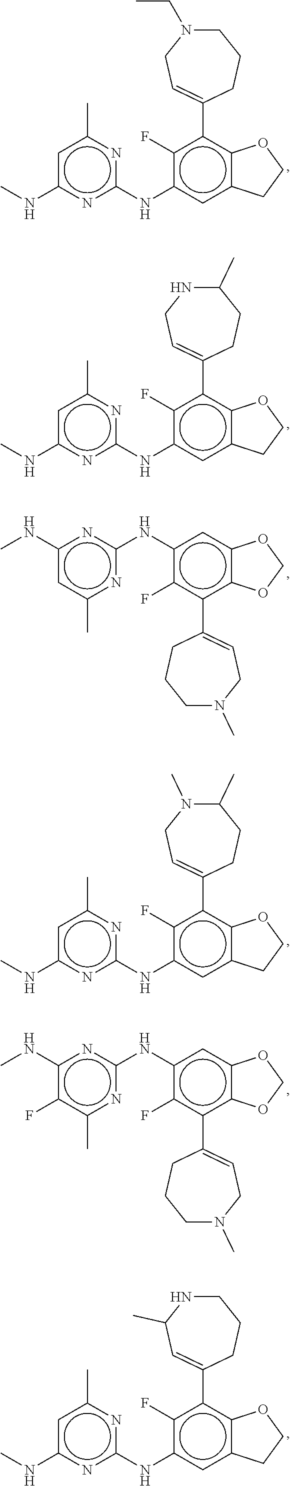

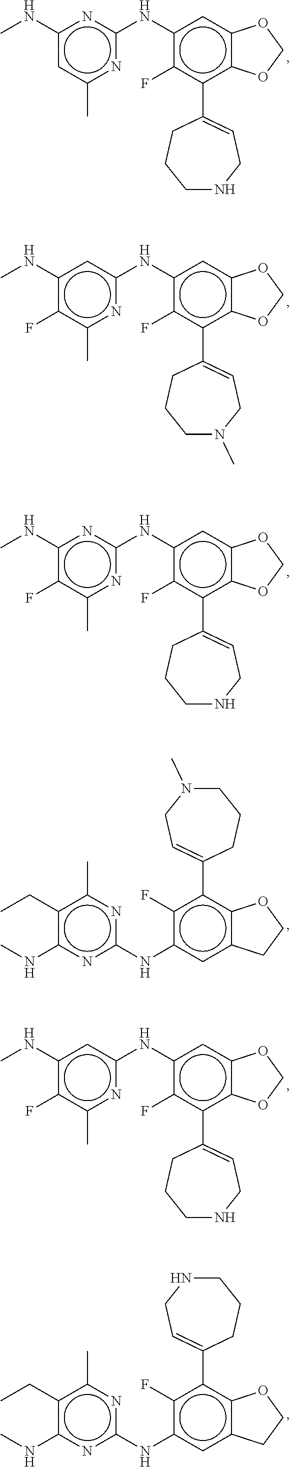

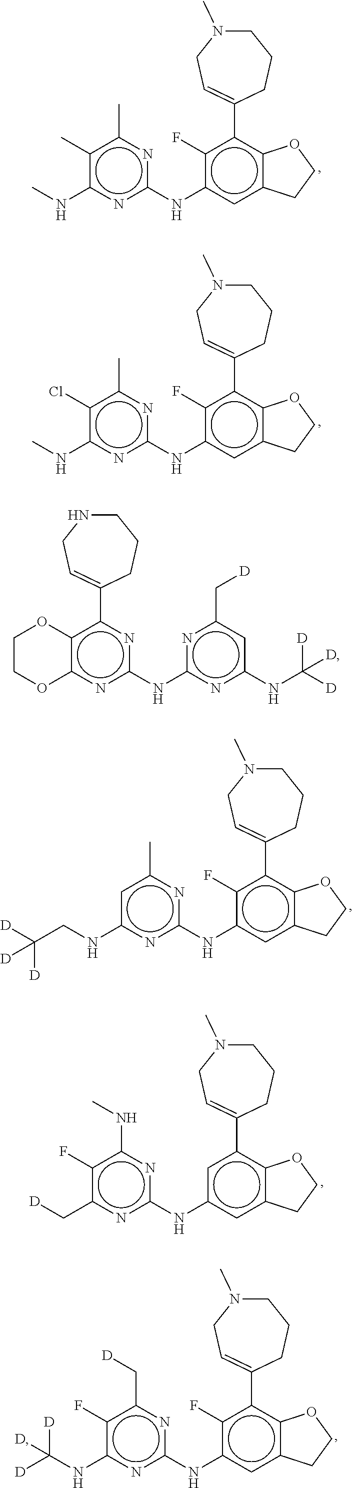

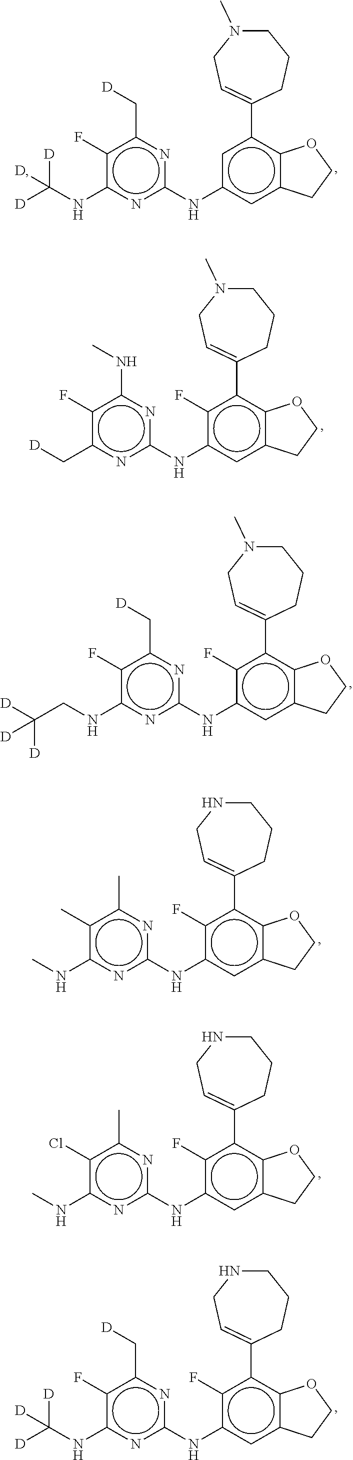

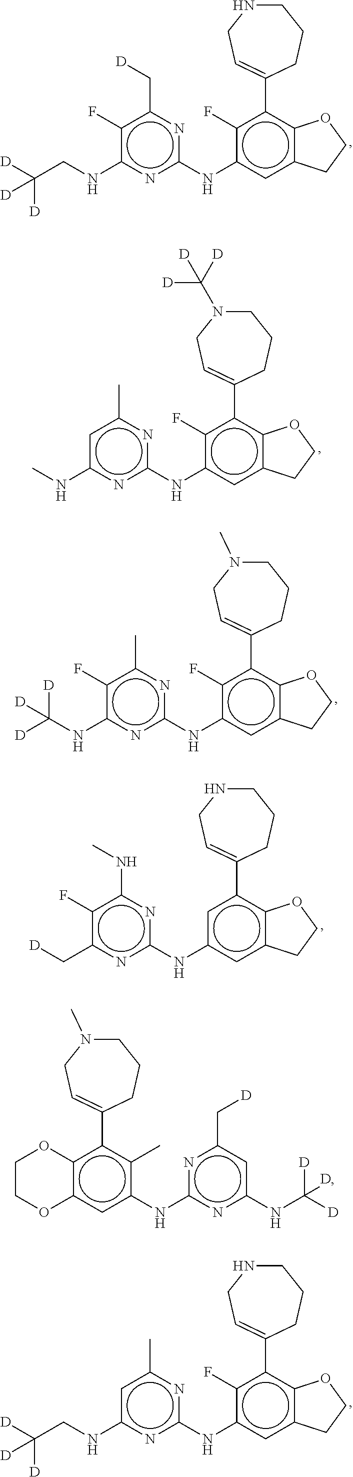

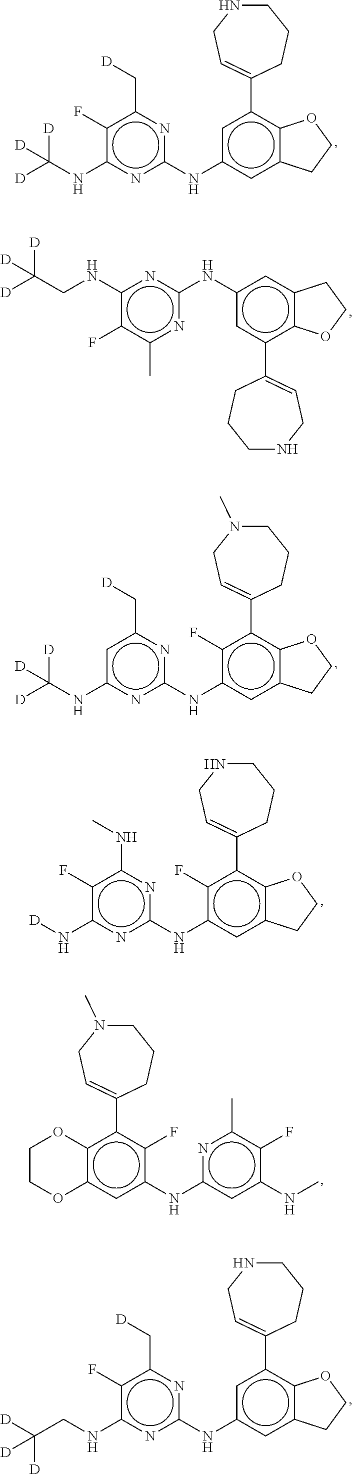

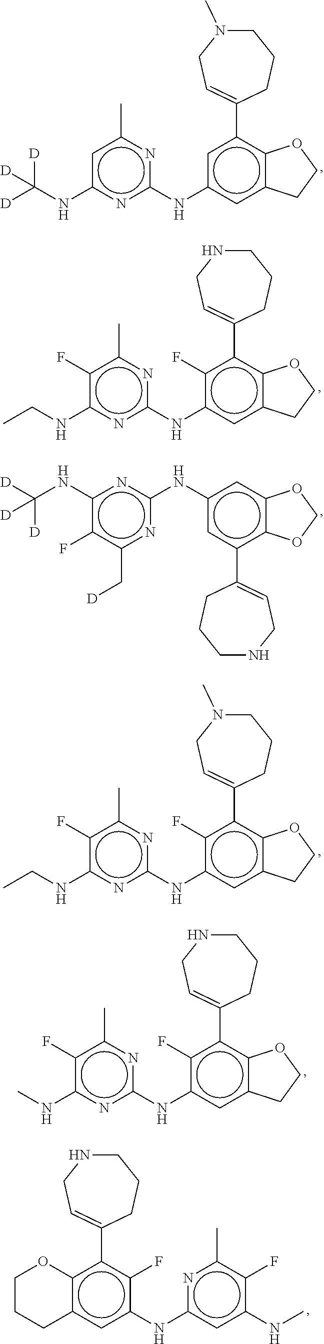

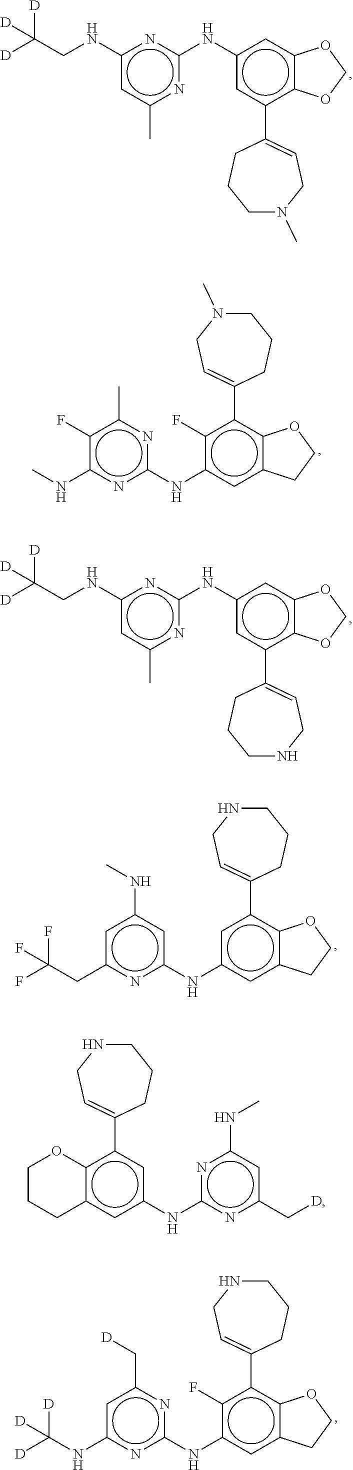

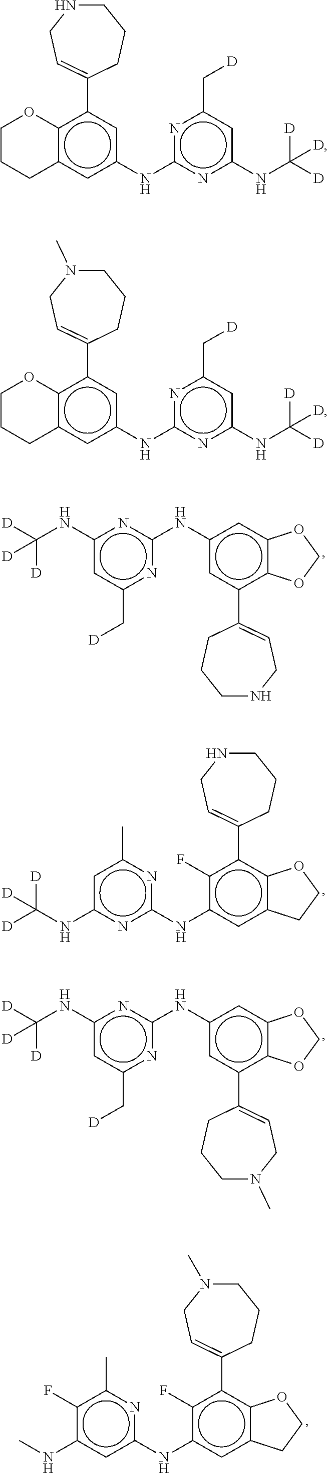

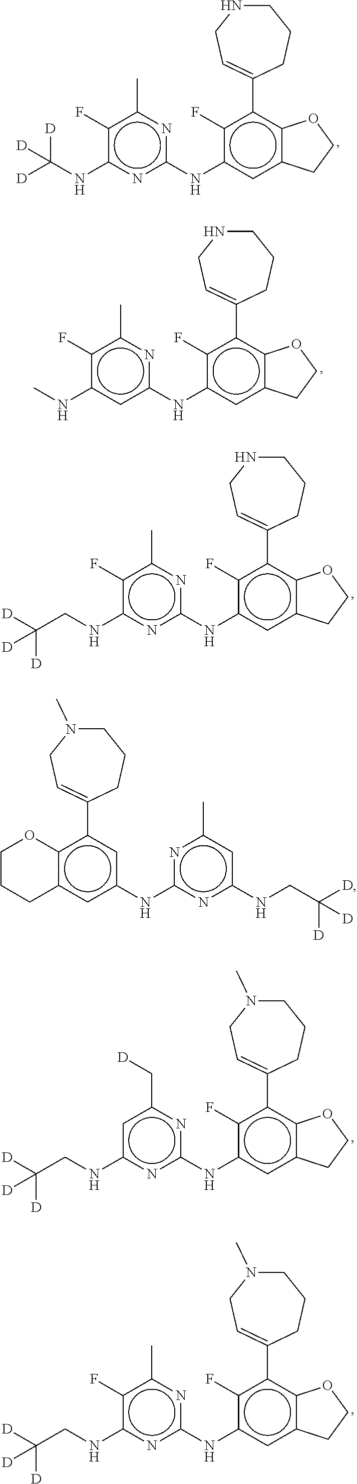

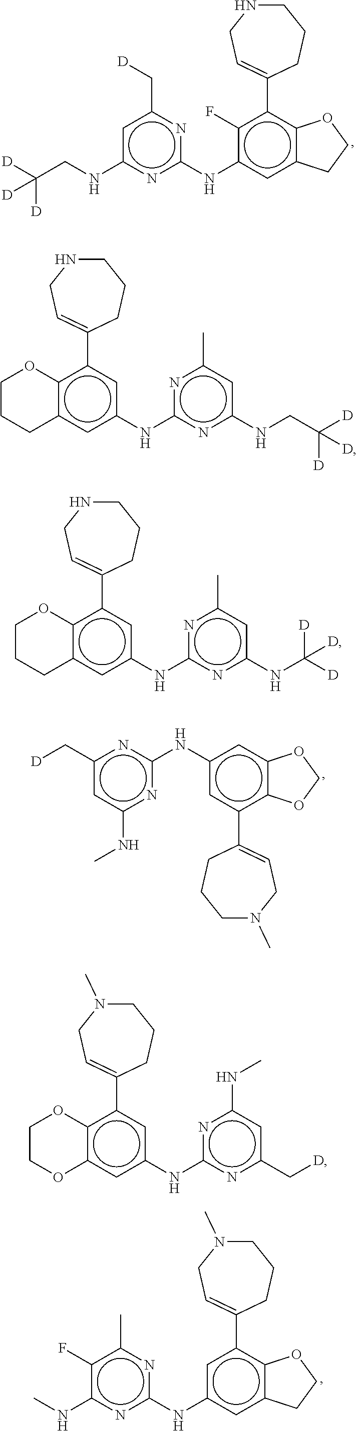

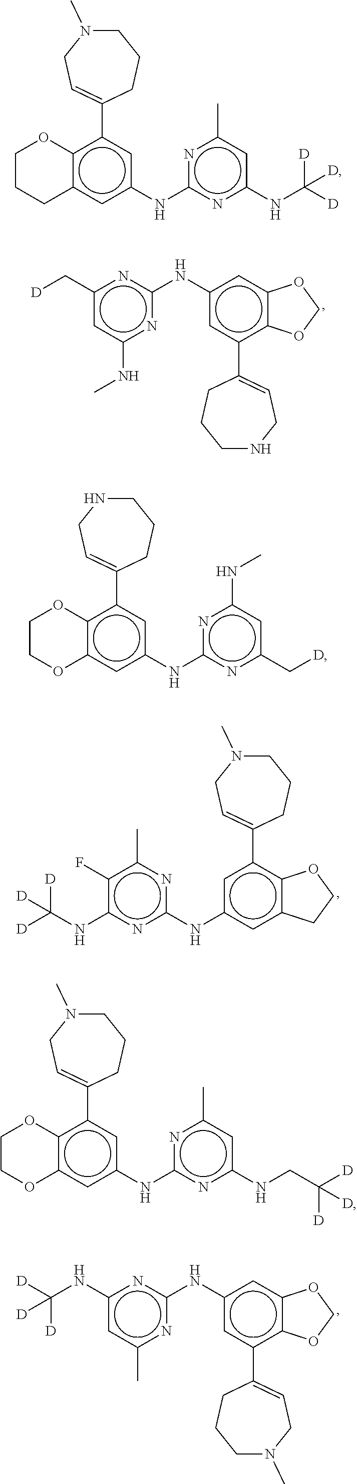

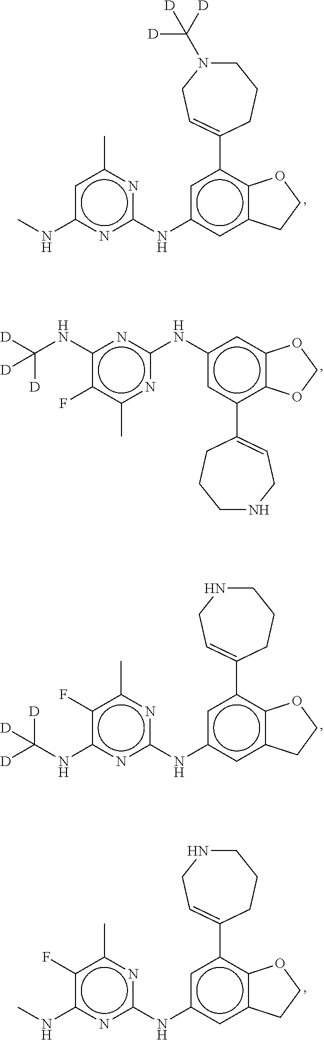

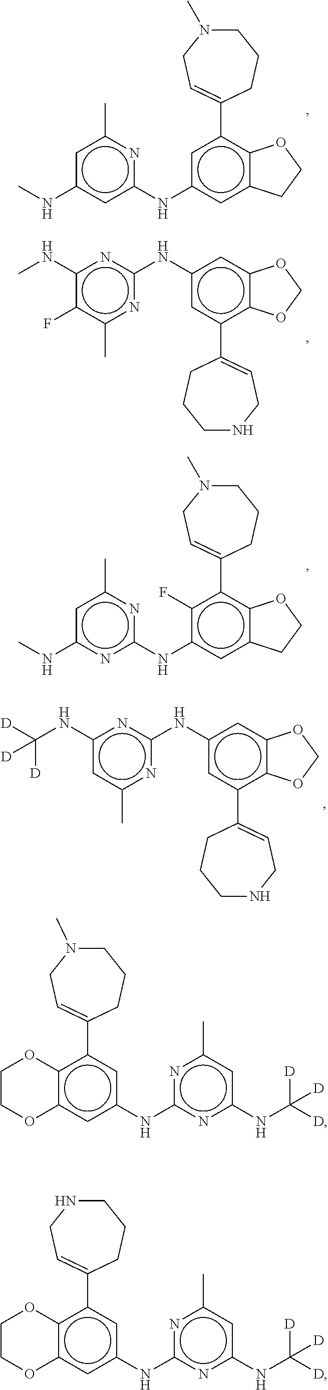

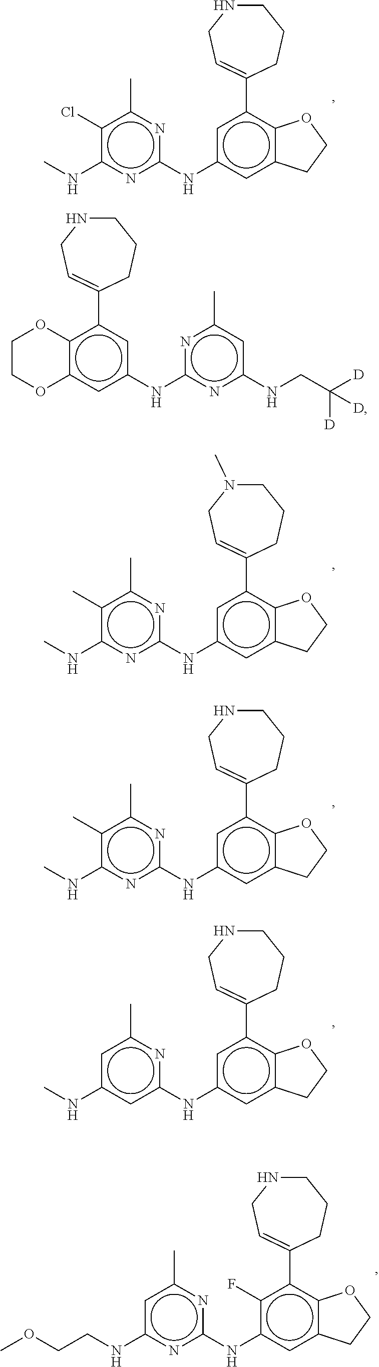

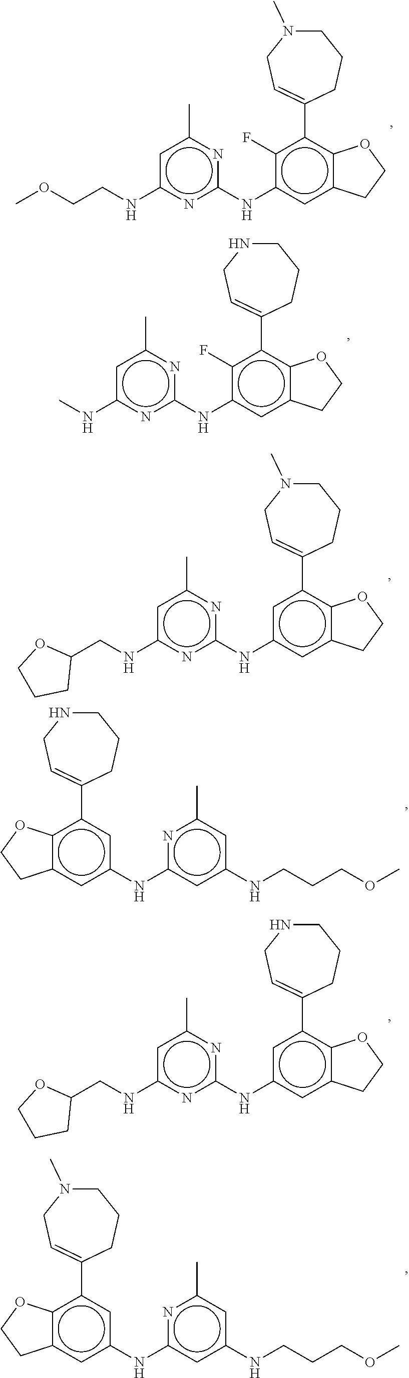

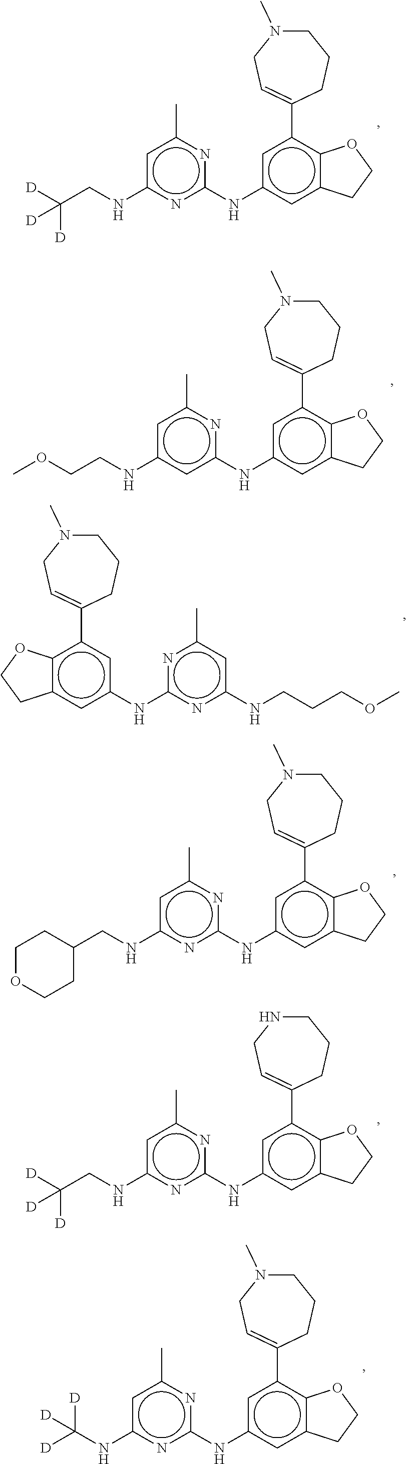

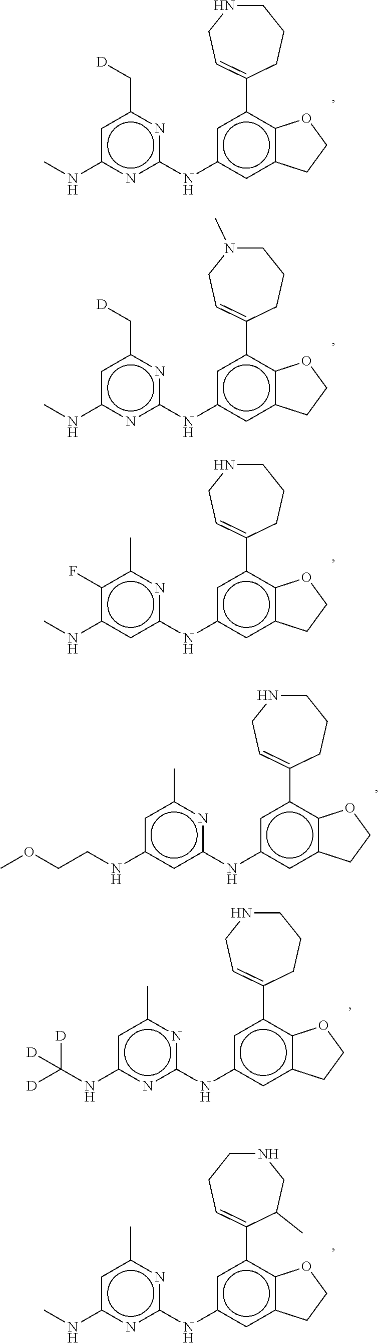

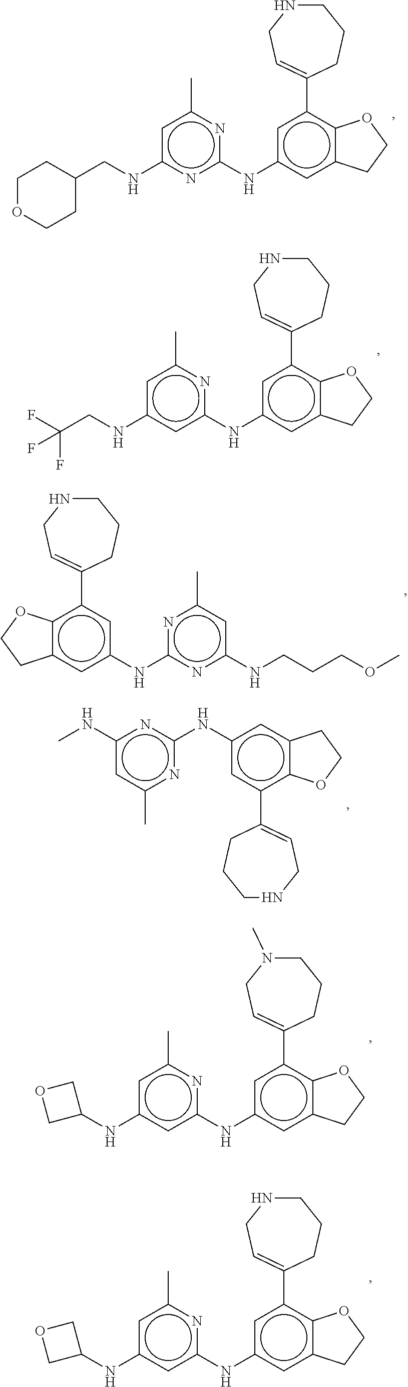

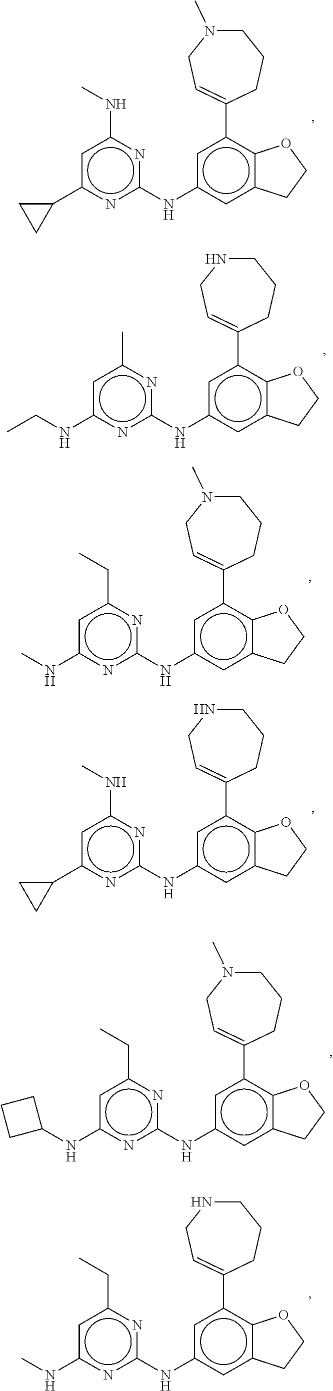

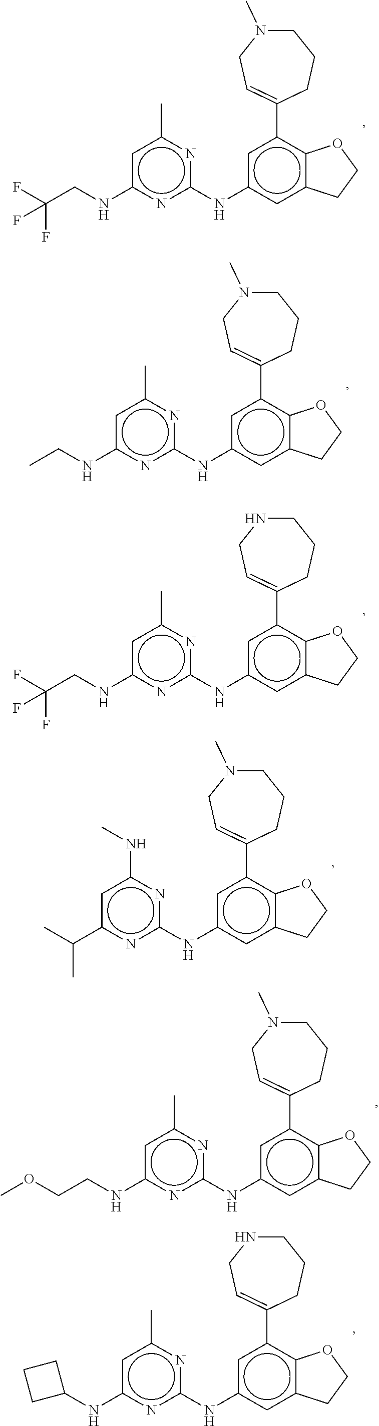

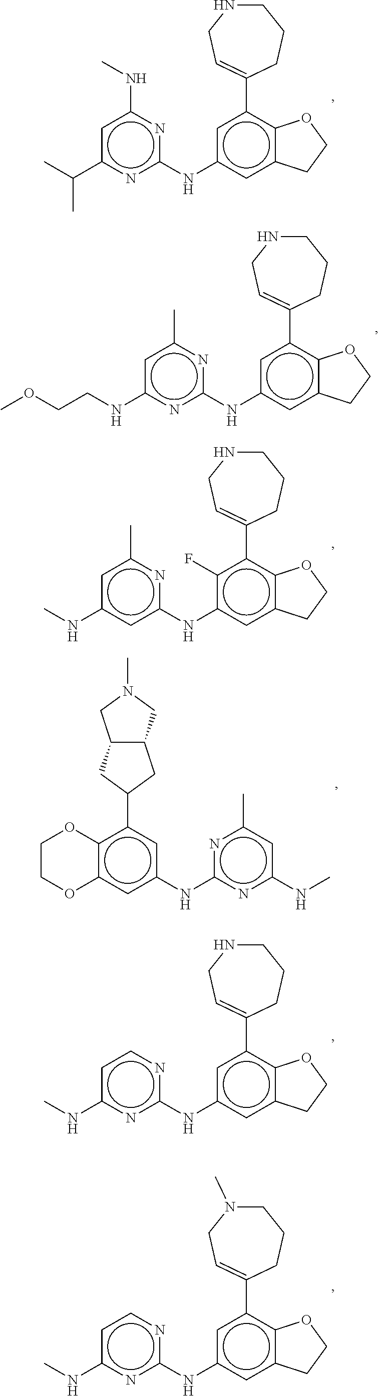

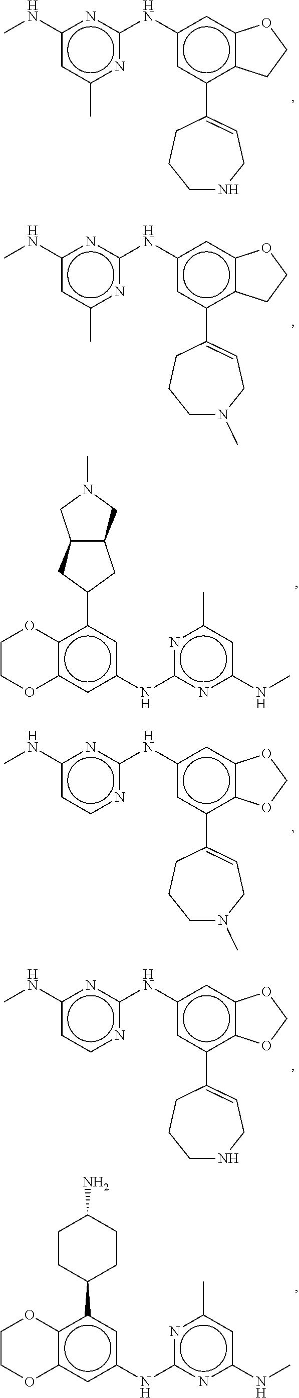

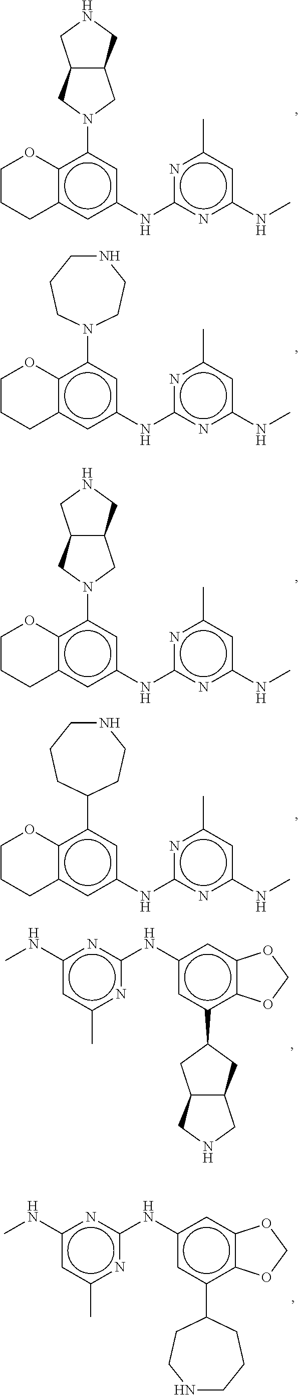

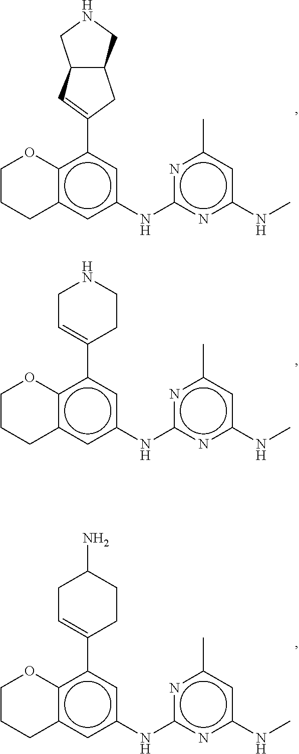

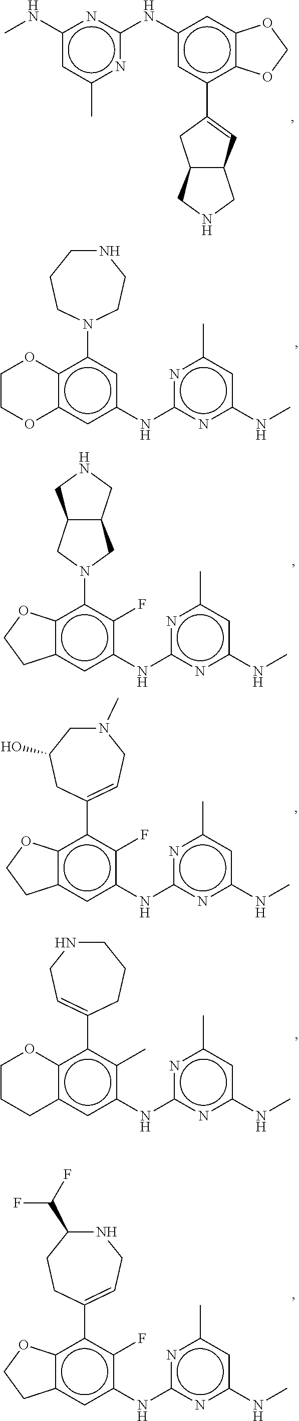

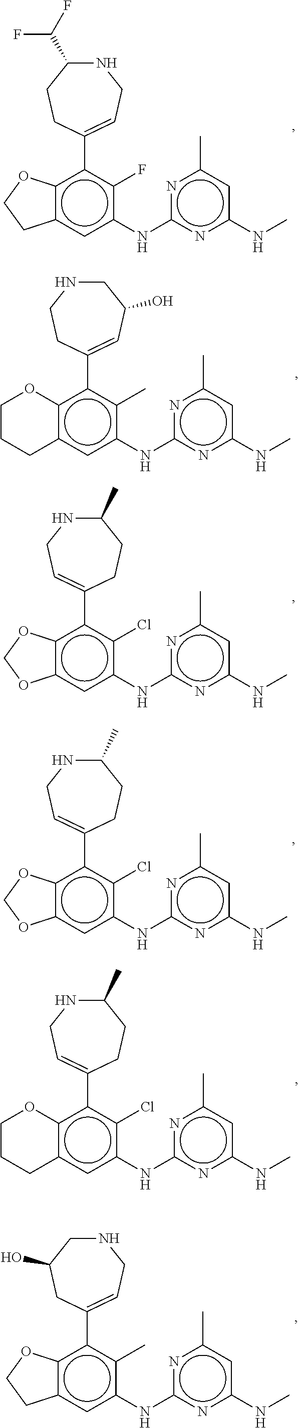

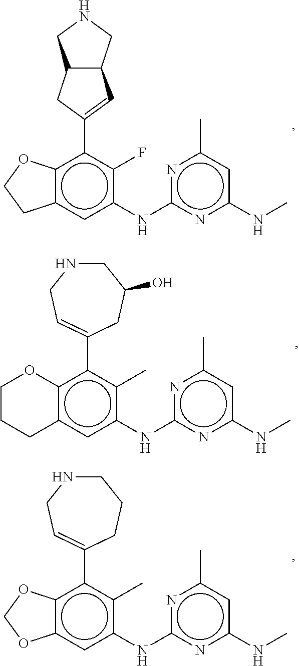

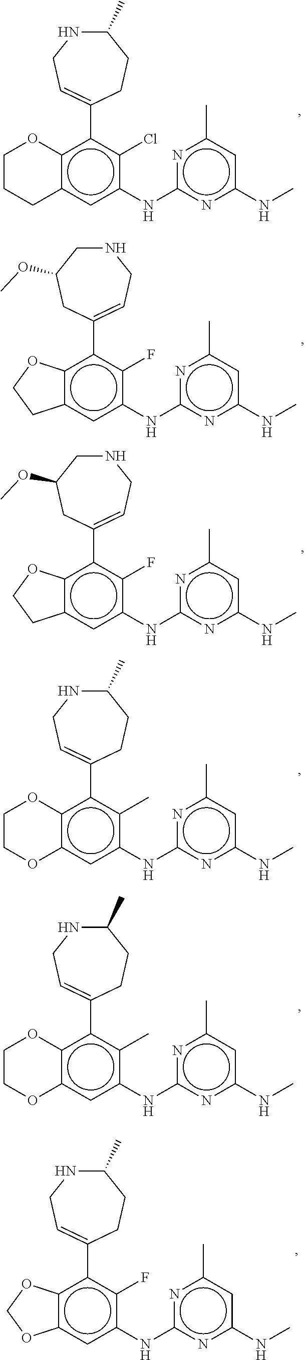

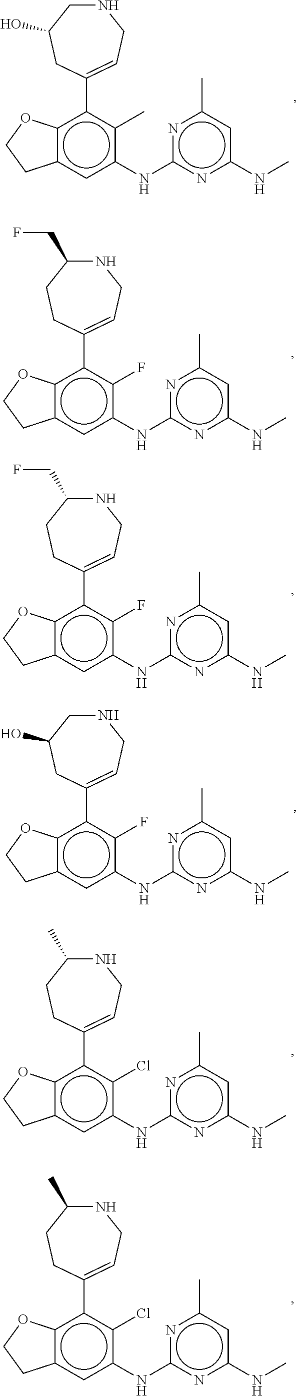

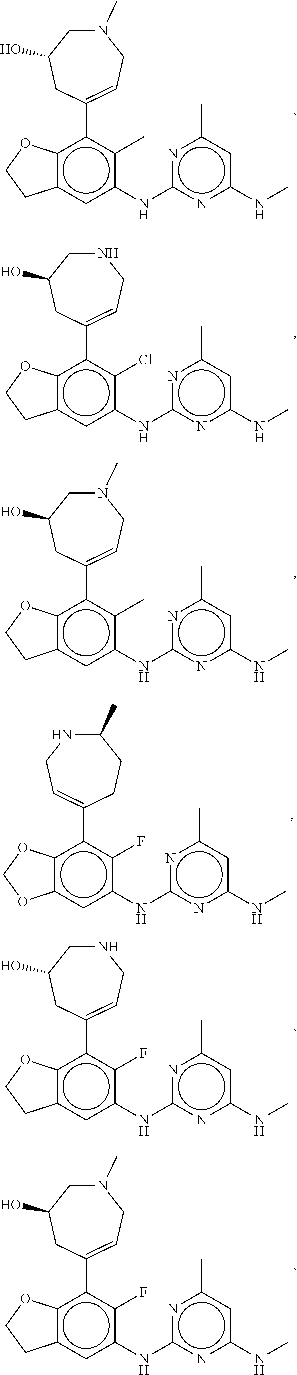

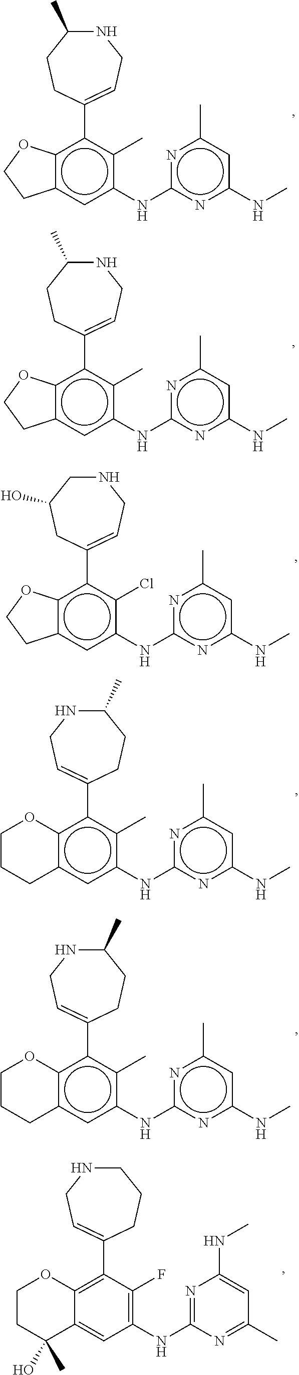

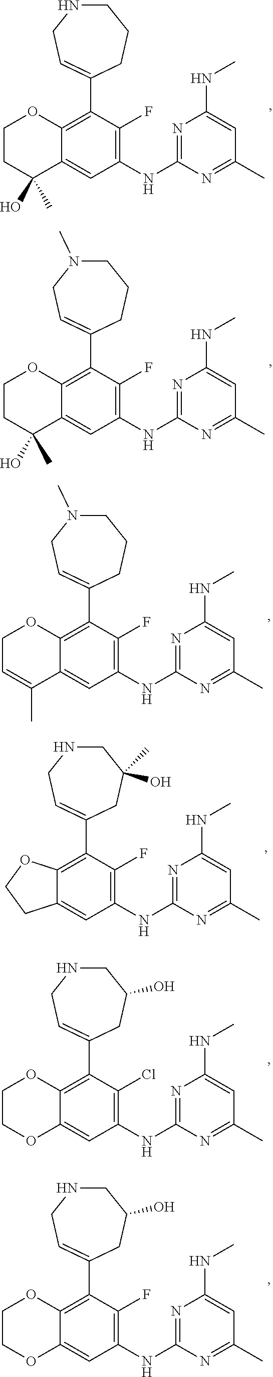

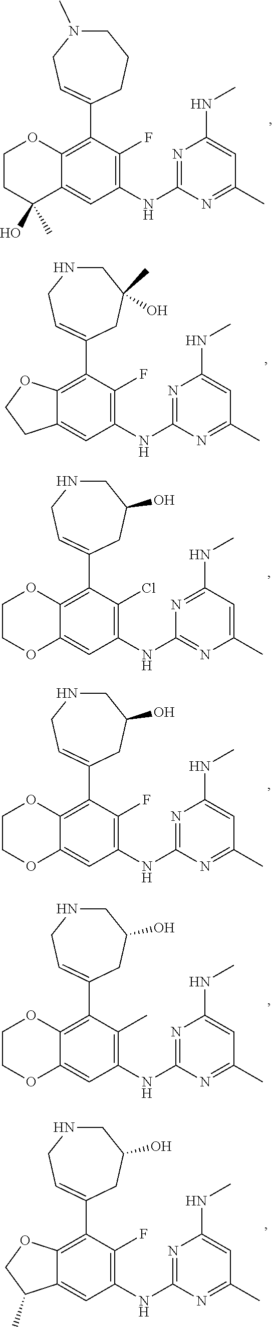

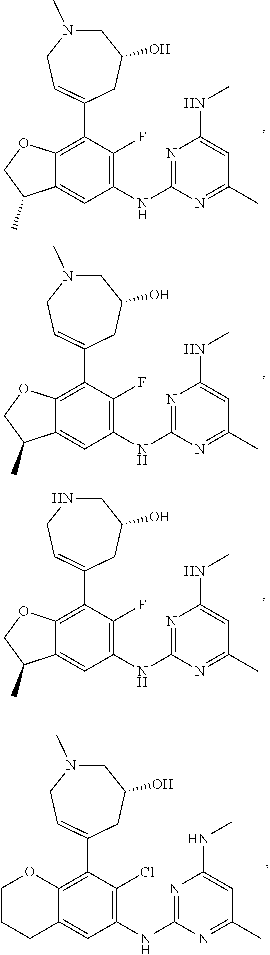

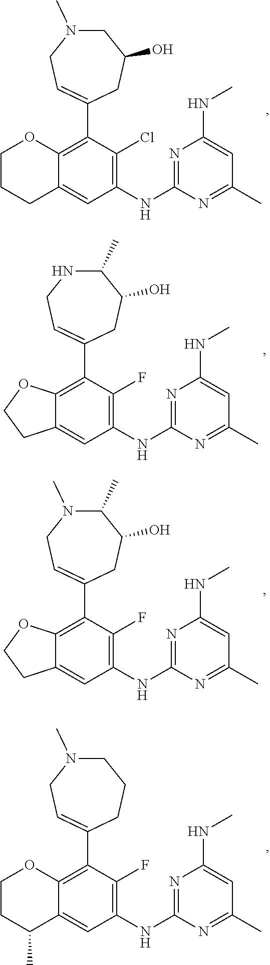

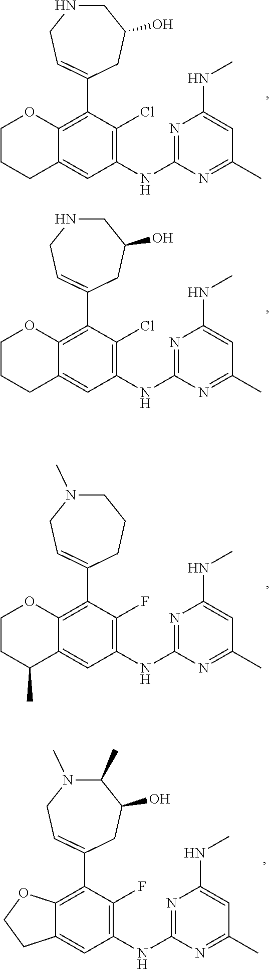

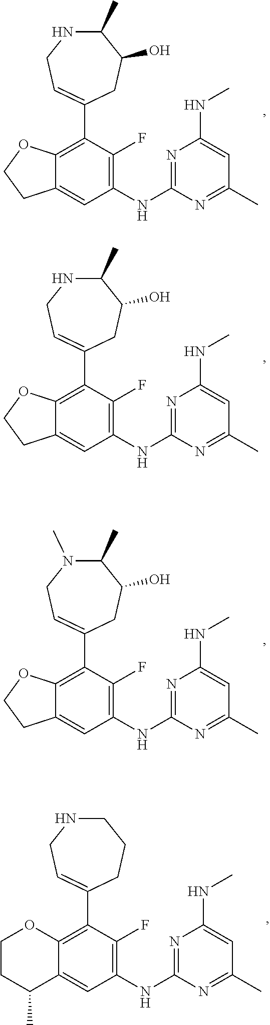 [SMILES: CNc1cc(C)nc(Nc2cc3c(c(C4=CCN[C@@H](C)[C@@H](O)C4)c2F)OCC3)n1 ; CNc1cc(C)nc(Nc2cc3c(c(C4=CCN[C@@H](C)[C@H](O)C4)c2F)OCC3)n1 ; CNc1cc(C)nc(Nc2cc3c(c(C4=CCN(C)[C@@H](C)[C@H](O)C4)c2F)OCC3)n1 ; CNc1cc(C)nc(Nc2cc3c(c(C4=CCNCCC4)c2F)OCC[C@H]3C)n1]

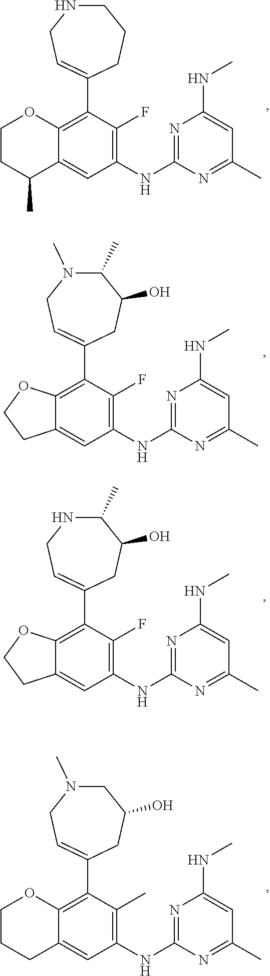

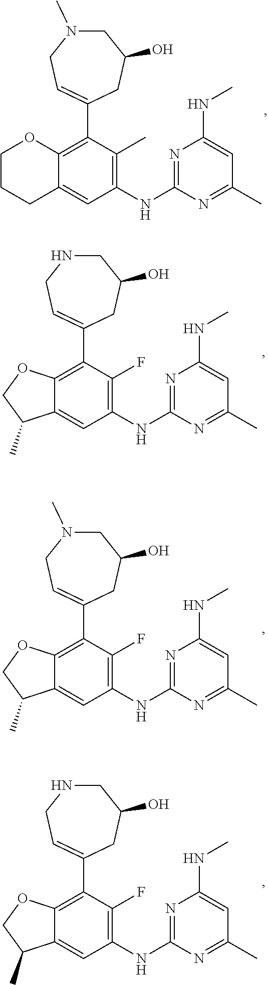 [SMILES: CNc1cc(C)nc(Nc2cc3c(c(C4=CCN(C)C[C@@H](O)C4)c2C)OCCC3)n1 ; CNc1cc(C)nc(Nc2cc3c(c(C4=CCNC[C@@H](O)C4)c2F)OC[C@H]3C)n1 ; CNc1cc(C)nc(Nc2cc3c(c(C4=CCN(C)C[C@@H](O)C4)c2F)OC[C@H]3C)n1 ; CNc1cc(C)nc(Nc2cc3c(c(C4=CCNC[C@@H](O)C4)c2F)OC[C@@H]3C)n1]

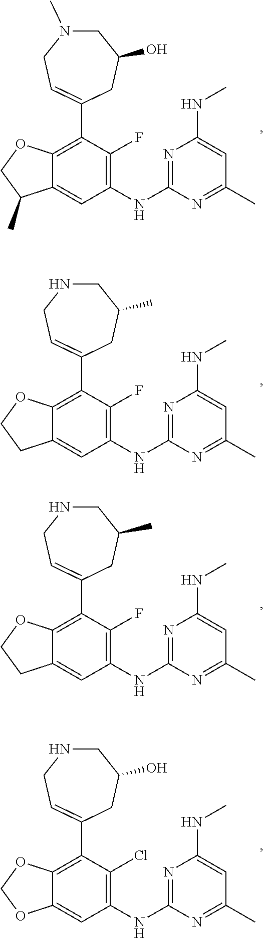 [SMILES: CNc1cc(C)nc(Nc2cc3c(c(C4=CCN(C)C[C@@H](O)C4)c2F)OC[C@@H]3C)n1 ; CNc1cc(C)nc(Nc2cc3c(c(C4=CCNC[C@H](C)C4)c2F)OCC3)n1 ; CNc1cc(C)nc(Nc2cc3c(c(C4=CCNC[C@@H](C)C4)c2F)OCC3)n1 ; CNc1cc(C)nc(Nc2cc3c(c(C4=CCNC[C@H](O)C4)c2Cl)OCO3)n1]

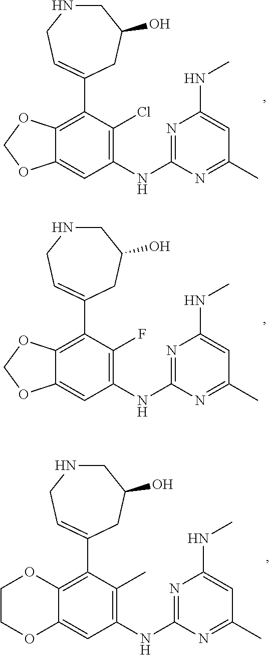 [SMILES: CNc1cc(C)nc(Nc2cc3c(c(C4=CCNC[C@@H](O)C4)c2Cl)OCO3)n1 ; CNc1cc(C)nc(Nc2cc3c(c(C4=CCNC[C@H](O)C4)c2F)OCO3)n1 ; CNc1cc(C)nc(Nc2cc3c(c(C4=CCNC[C@@H](O)C4)c2C)OCCO3)n1]

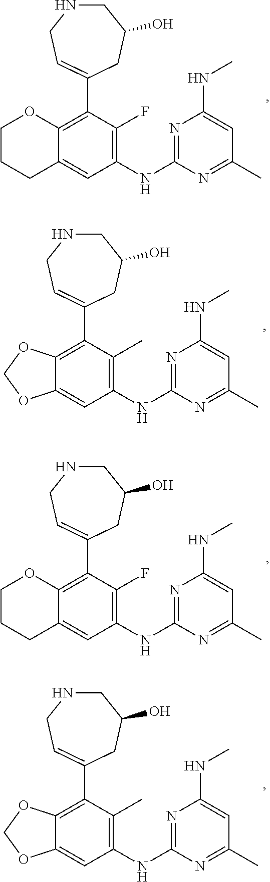 [SMILES: CNc1cc(C)nc(Nc2cc3c(c(C4=CCNC[C@H](O)C4)c2F)OCCC3)n1 ; CNc1cc(C)nc(Nc2cc3c(c(C4=CCNC[C@H](O)C4)c2C)OCO3)n1 ; CNc1cc(C)nc(Nc2cc3c(c(C4=CCNC[C@@H](O)C4)c2F)OCCC3)n1 ; CNc1cc(C)nc(Nc2cc3c(c(C4=CCNC[C@@H](O)C4)c2C)OCO3)n1]

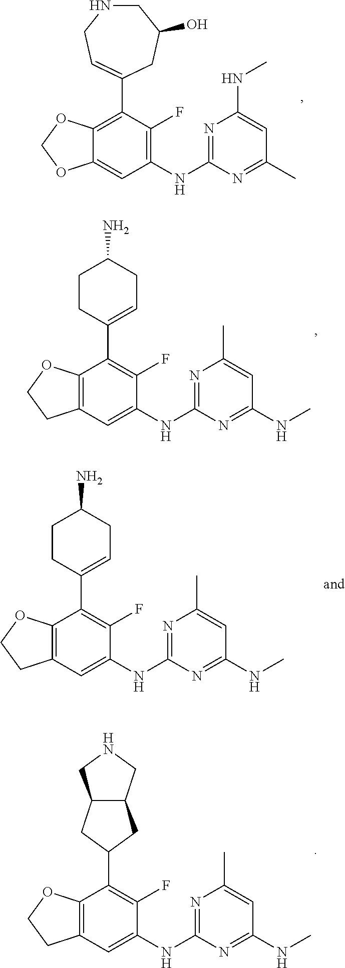

31. A composition comprising a compound of any one of claims 1-30, or a pharmaceutically acceptable salt thereof, and a pharmaceutically acceptable carrier.

32. A compound of any one of claims 1-30, or a pharmaceutically acceptable salt thereof, or a composition of claim 31 for use in treating a disease or disorder that can be treated by modulation of EHMT1 or EHMT2.

33. The compound for use of claim 32, wherein the disease or disorder is selected from the group consisting of cancer, sickle cell disease, and beta thalassemia.

34. The compound for use of claim 33, wherein the disease or disorder is cancer.

35. The compound for use of claim 34, wherein the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

36. The compound for use of claim 35, wherein the cancer is selected from the group consisting of a melanoma, bladder cancer, colorectal cancer, head and neck cancer, esophageal cancer, liver cancer, lung cancer, pancreas cancer, and stomach cancer.

37. The compound for use of any one of claims 32-36, wherein the use comprises a combination of compound of any one of claims 1-30, or a pharmaceutically acceptable salt thereof, or a composition of claim 31 and at least one additional therapeutic agent.

38. The compound for use of claim 37, wherein the at least one additional therapeutic agent is chemotherapy or radiation.
